(19) United States
(12) Patent Application Publication
(10) Pub. No.: US 2025/0319200 A1
(43) Pub. Date: Oct. 16, 2025

(54) ADENOVIRAL GENE THERAPY VECTORS

(71) Applicant: Ensoma, Inc., Boston, MA (US)

(72) Inventors: Ashvin Reddy Bashyam, Cambridge, MA (US); Soumitra Roy, Townsend, DE (US); Robert Thomas Peters, Needham, MA (US); Kush M. Parmar, Brookline, MA (US)

(21) Appl. No.: 18/554,883

(22) PCT Filed: Apr. 15, 2022
(86) PCT No.: PCT/US2022/025081

§ 371 Date: Oct. 11, 2023

Related U.S. Application Data
(60) Provisional application No. 63/175,249, filed on Apr. 15, 2021.

(51) Int. Cl. A61K 48/00; A61K 31/155; A61K 31/522; A61K 45/06; C07K 14/005; C12N 15/86

(52) U.S. Cl. CPC A61K 48/0041; A61K 31/155; A61K 31/522; A61K 45/06; C07K 14/005; C12N 15/86; C12N 2710/10022; C12N 2710/10043

(57) ABSTRACT

The present disclosure includes adenoviral vectors characterized by efficient transduction of hematopoietic cells (e.g., one or more particular types of hematopoietic cells), e.g., for in vivo or ex vivo gene therapy. The present disclosure includes, among other things, Ad3, Ad5, Ad7, Ad11, Ad14, Ad16, Ad21, Ad34, Ad35, Ad37, and Ad50 vectors and genomes. Ad3, Ad5, Ad7, Ad11, Ad14, Ad16, Ad21, Ad34, Ad35, Ad37, and Ad50 vectors and genomes of the present disclosure can include therapeutic payloads.

### PRIORITY APPLICATION

The present application is a National Stage of International Application No. PCT/US2022/025081, filed Apr. 15, 2022, which claims the benefit of U.S. Provisional Patent Application No. 63/175,249, filed Apr. 15, 2021, the content of each of which is hereby incorporated by reference herein in its entirety.

### SEQUENCE LISTING

The instant application contains a Sequence Listing, which has been submitted electronically as a .txt file named “2013585-0129_SL.txt”. The .txt file was created on Aug. 4, 2022 and is 1,595,680 bytes in size. The entire contents of the Sequence Listing are herein incorporated by reference.

### BACKGROUND

Many medical conditions are caused by genetic mutation and/or are treatable, at least in part, by gene therapy. Some conditions are particularly treatable by modification of hematopoietic cells. Compositions and methods that target hematopoietic cells for gene therapy are therefore needed.

### SUMMARY

Gene therapy can treat many conditions that have a genetic component, including without limitation hemoglobinopathies, immune deficiencies, and cancers. In various gene therapies, hematopoietic cells are an important target. However, current methods and compositions for modifying hematopoietic cells are limited. For instance, there is a need to identify vectors that selectively target hematopoietic cells (e.g., one or more particular types of hematopoietic cells). The present disclosure includes the recognition that certain adenoviral vectors selectively target hematopoietic cells (e.g., one or more particular types of hematopoietic cells).

The present disclosure includes, among other things, adenoviral vectors that selectively target hematopoietic cells of various types provided herein. The present disclosure includes, among other things, adenoviral vectors that selectively target hematopoietic stem cells (HSCs, e.g., CD34⁺ long-term (LT)-HSCs and/or CD34⁺ short-term (ST)-HSCs), common lymphoid progenitors (CLPs), T cells, NK cells, colony forming unit (CFU)-pre B cells, B cells, common myeloid progenitors (CMPs), granulocyte-macrophage progenitors (GMPs), CFU-M cells, monoblasts, monocytes, macrophages, CFU-G cells, myeloblasts, granulocytes, neutrophils, eosinophils, basophils, megakaryocyte-erythrocyte progenitors (MEPs), BFU-E cells, CFU-E cells, erythroblasts, erythrocytes, CFU-Mk cells, megakaryocytes, and/or platelets. The present disclosure includes, among other things, adenoviral vectors that selectively target CD34⁺ hematopoietic cells.

The present disclosure includes, among other things, Ad3, 5, 7, 11, 14, 16, 21, 34, 35, 37, or 50 vectors and Ad3, 5, 7, 11, 14, 16, 21, 34, 35, 37, or 50 genomes (e.g., “recombinant” or “engineered” adenoviral vectors and adenoviral genomes) that selectively target one or more hematopoietic cell types. Ad3, 5, 7, 11, 14, 16, 21, 34, 35, 37, or 50 vectors and genomes of the present disclosure can include various payloads. In various embodiments, a payload can include one or more of a nucleic acid sequence encoding a CRISPR system, base editing system, prime editing system, or other expression product. The present disclosure includes, among other things, combination adenoviral vectors and adenoviral genomes that include nucleic acid sequences encoding a plurality of expression products that together contribute to treatment of a disease or condition. The present disclosure includes, among other things, Ad3, 5, 7, 11, 14, 16, 21, 34, 35, 37, or 50 adenoviral vectors and Ad3, 5, 7, 11, 14, 16, 21, 34, 35, 37, or 50 adenoviral genomes for integration of a nucleic acid payload into a target cell genome. The present disclosure includes, among other things, Ad3, 5, 7, 11, 14, 16, 21, 34, 35, 37, or 50 donor vectors, Ad3, 5, 7, 11, 14, 16, 21, 34, 35, 37, or 50 adenoviral donor genomes, helper dependent Ad3, 5, 7, 11, 14, 16, 21, 34, 35, 37, or 50 adenoviral donor vectors, helper dependent Ad3, 5, 7, 11, 14, 16, 21, 34, 35, 37, or 50 adenoviral donor genomes, support vectors, support genomes, Ad3, 5, 7, 11, 14, 16, 21, 34, 35, 37, or 50 helper vectors, and Ad3, 5, 7, 11, 14, 16, 21, 34, 35, 37, or 50 helper genomes. For avoidance of doubt, a list of serotypes such as “Ad3, 5, 7, 11, 14, 16, 21, 34, 35, 37, or 50” can alternatively be written as “Ad3, Ad5, Ad7, Ad1, Ad14, Ad16, Ad21, Ad34, Ad35, Ad37, or Ad50.”

In at least one aspect, the present disclosure provides method of selectively targeting a hematopoietic cell type, the method including administering to a subject or system an adenoviral vector, where the adenoviral vector includes: (a) a capsid including one or more viral polypeptides of an Ad3, Ad5, Ad7, Ad11, Ad14, Ad16, Ad21, Ad34, Ad35, Ad37, or Ad50 serotype, where the one or more viral polypeptides include one or more of a: (i) fiber knob; (ii) fiber shaft; (iii) fiber tail; (iv) penton; and (v) hexon; and (b) a double-stranded DNA genome including a heterologous nucleic acid payload. In various embodiments, the genome further includes: (a) a 3′ ITR and a 5′ ITR, where each of the 3′ ITR and the 5′ ITR are of the viral polypeptide serotype; and (b) a packaging sequence, where the packing sequence is of the viral polypeptide serotype.

In various embodiments, the hematopoietic cell type is or includes a terminally differentiated cell type. In various embodiments, the hematopoietic cell type is or includes a progenitor cell type. In various embodiments, the hematopoietic cell type is or includes HSCs, common lymphoid progenitors (CLPs), T cells, NK cells, colony forming unit (CFU)-pre B cells, B cells, common myeloid progenitors (CMPs), granulocyte-macrophage progenitors (GMPs), CFU-M cells, monoblasts, monocytes, macrophages, CFU-G cells, myeloblasts, granulocytes, neutrophils, eosinophils, basophils, megakaryocyte-erythrocyte progenitors (MEPs), BFU-E cells, CFU-E cells, erythroblasts, erythrocytes, CFU-Mk cells, megakaryocytes, and/or platelets, optionally where the HSCs are CD34⁺ long-term hematopoietic stem cells (LT-HSCs) and/or CD34⁺ short-term (ST)-HSCs.

In various embodiments, the method is a method of in vivo gene therapy. In various embodiments, the hematopoietic cell type is a mammalian hematopoietic cell type, optionally where the mammalian hematopoietic cell type is a human hematopoietic cell type. In various embodiments, the subject is a mammalian subject, optionally where the mammalian subject is a human subject. In various embodiments, the method includes mobilization of hematopoietic cells of the subject prior to administration of the adenoviral vector. In various embodiments, the method includes administering one or more immunosuppression agents to the subject, optionally where the administration of the one or more immunosuppression agents is prior to the administration of the adenoviral vector.

In various embodiments, the method is a method of ex vivo gene therapy. In various embodiments, the hematopoietic cell type is a mammalian hematopoietic cell type, optionally where the mammalian hematopoietic cell type is a human hematopoietic cell type. In various embodiments, the system is or includes a biological sample derived from a mammalian donor, optionally where the mammalian donor is a human donor. In various embodiments, the heterologous nucleic acid payload includes a selectable marker, optionally where the selectable marker is MGMT^{P140K}. In various embodiments, the method includes administering a selecting agent to the subject, optionally where the selecting agent includes O⁶BG and/or BCNU.

In various embodiments, the one or more viral polypeptides include the: (a) fiber knob and fiber shaft; (b) fiber knob and fiber tail; (c) fiber knob and penton; (d) fiber knob and hexon; (e) fiber knob, hexon, and penton; (f) fiber shaft and fiber tail; (g) fiber shaft and penton; (h) fiber shaft and hexon; (i) fiber shaft, hexon, and penton; (j) fiber tail and penton; (k) fiber tail and hexon; (l) fiber tail, hexon, and penton; (m) fiber knob, fiber shaft, and fiber tail; (n) fiber knob, fiber shaft, and penton; (o) fiber knob, fiber shaft, and hexon; (p) fiber knob, fiber shaft, hexon, and penton; (q) fiber knob, fiber shaft, fiber tail, and penton; (r) fiber knob, fiber shaft, fiber tail, penton, and hexon; or (s) penton and hexon. In various embodiments, the fiber knob has a sequence that has at least 80% identity to a sequence selected from SEQ ID NOs: 15, 33, 51, 69, 87, 105, 123, 141, 159, 177, and 195. In various embodiments, the fiber shaft has a sequence that has at least 80% identity to a sequence selected from SEQ ID NOs: 14, 32, 50, 68, 86, 104, 122, 140, 158, 176, and 194. In various embodiments, the fiber tail has a sequence that has at least 80% identity to a sequence selected from SEQ ID NOs: 18, 36, 54, 72, 90, 108, 126, 144, 162, 180, and 198. In various embodiments, the penton has a sequence that has at least 80% identity to a sequence selected from SEQ ID NOs: 16, 34, 52, 70, 88, 106, 124, 142, 160, 178, and 196. In various embodiments, the hexon has a sequence that has at least 80% identity to a sequence selected from SEQ ID NOs: 17, 35, 53, 71, 89, 107, 125, 143, 161, 179, and 197. In various embodiments, the adenoviral vector includes a fiber of the serotype of the viral peptides. In various embodiments, the fiber has a sequence that has at least 80% identity to a sequence selected from SEQ ID NOs: 13, 31, 49, 67, 85, 103, 121, 139, 157, 175, and 193. In various embodiments, the adenoviral vector is a chimeric vector characterized in that the capsid includes at least one of a fiber knob, fiber shaft, fiber tail, hexon, or penton that is not of the serotype of the viral peptides. In various embodiments, the adenoviral vector is a helper dependent vector.

In various embodiments, the heterologous nucleic acid payload encodes a protein. In various embodiments, the heterologous nucleic acid payload encodes a chimeric antigen receptor (CAR), T cell receptor (TCR), antibody, or small RNA, optionally where the small RNA is an shRNA. In various embodiments, the heterologous nucleic acid payload encodes a chimeric antigen receptor (CAR) or T cell receptor (TCR) and the hematopoietic cell type is or includes T cells. In various embodiments, the heterologous nucleic acid payload encodes an antibody and the hematopoietic cell type is or includes B cells. In various embodiments, the heterologous nucleic acid payload encodes a gene editing enzyme or system, where the gene editing is selected from CRISPR editing, base editing, prime editing, and zinc finger nuclease editing.

In various embodiments, the heterologous nucleic acid payload encodes an agent for treatment of a condition selected from adenosine deaminase deficiency (ADA), adrenoleukodystrophy (ALD), agammaglobulinemia, alpha-1 antitrypsin deficiency, congenital amegakaryocytic thrombocytopenia, amyotrophic lateral sclerosis (Lou Gehrig's disease), ataxia telangiectasia, Batten disease, Bernard-Soulier Syndrome, CD40/CD40L deficiency, chronic granulomatous disease, common variable immune deficiency (CVID), congenital thrombotic thrombocytopenic purpura (cTTP), cystic fibrosis, Diamond Blackfan anemia (DBA), DOCK 8 deficiency, dyskeratosis congenital, Fabry disease, Factor V Deficiency, Factor VII Deficiency, Factor X Deficiency, Factor XI Deficiency, Factor XII Deficiency, Factor XIII Deficiency, familial apolipoprotein E deficiency and atherosclerosis (ApoE), familial erythrophagocytic lymphohistiocytosis, Fanconi anemia (FA), Friedreich ataxia, Gaucher disease, Glanzmann thrombasthenia, glucosemia, glycogen storage disease, glycogen storage disease type I (GSDI), Gray Platelet Syndrome, hemophilia, hemophilia A, hemophilia B, hereditary hemochromatosis, Hurler's syndrome, hyper IgM, Hypogammaglobulinemia, Krabbe disease, major histocompatibility complex class II deficiency (MHC-II), maple syrup urine disease, metachromatic leukodystrophy (MLD), mucopolysaccharidoses, mucopolysaccharidosis type I (MPS I), MPS II (Hunter Syndrome), MPS III (Sanfilippo syndrome), MPS IV (Morquio syndrome), MPS V, MPS VI (Maroteaux-Lamy syndrome), MPS VII (sly syndrome), muscular dystrophy, Niemann-Pick disease, Parkinson's disease, paroxysmal nocturnal hemoglobinuria (PNH), pernicious anemia, phenylketonuria (PKU), Pompe disease, pulmonary alveolar proteinosis (PAP), pure red cell aplasia (PRCA), pyruvate kinase deficiency, refractory anemia, Shwachman-Diamond syndrome, selective IgA deficiency, severe aplastic anemia, severe combined immunodeficiency disease (SCID), Severe combined immunodeficiency due to adenosine deaminase deficiency (ADA-SCID), sickle cell anemia, sickle cell disease, sickle cell trait, Tay Sachs, thalassemia, thalassemia intermedia, von Gierke disease, von Willebrand Disease, Wiskott-Aldrich syndrome (WAS), X-linked agammaglobulinemia (XLA), X-linked severe combined immunodeficiency (SCID-X1), Zellweger syndrome, α-mannosidosis, β-mannosidosis, β-thalassemia, and/or β-thalassemia major.

In various embodiments, the capsid includes one or more viral polypeptides of an Ad5, Ad7, Ad11, Ad16, Ad34, or Ad35 serotype, and the hematopoietic cell type is or includes monocytes. In various embodiments, the capsid includes one or more viral polypeptides of an Ad11, Ad16, Ad34, or Ad35 serotype, and the hematopoietic cell type is or includes monocytes. In various embodiments, the capsid includes one or more viral polypeptides of an Ad11, Ad34, or Ad35 serotype, and the hematopoietic cell type is or includes monocytes. In various embodiments, the monocytes are CD11+/CD14+ monocytes.

In various embodiments, the capsid includes one or more viral polypeptides of an Ad5, Ad7, Ad11, Ad16, Ad34, or Ad35 serotype, and the hematopoietic cell type is or includes T cells. In various embodiments, the capsid includes one or more viral polypeptides of an Ad5, Ad16, Ad34, or Ad35 serotype, and the hematopoietic cell type is or includes T cells. In various embodiments, the capsid includes one or more viral polypeptides of an Ad34 or Ad35 serotype, and the hematopoietic cell type is or includes T cells. In various embodiments, the T cells are CD3+ T cells.

In various embodiments, the capsid includes one or more viral polypeptides of an Ad5, Ad7, Ad11, Ad16, Ad34, or Ad35 serotype, and the hematopoietic cell type is or includes NK cells. In various embodiments, the capsid includes one or more viral polypeptides of an Ad11, Ad16, Ad34 or Ad35 serotype, and the hematopoietic cell type is or includes NK cells. In various embodiments, the capsid includes one or more viral polypeptides of an Ad11, Ad34, or Ad35 serotype, and the hematopoietic cell type is or includes NK cells. In various embodiments, the NK cells are CD3−/CD56+ NK cells.

In various embodiments, the capsid includes one or more viral polypeptides of an Ad5, Ad7, Ad11, Ad16, Ad34, or Ad35 serotype, and the hematopoietic cell type is or includes B cells. In various embodiments, the capsid includes one or more viral polypeptides of an Ad16 serotype, and the hematopoietic cell type is or includes B cells. In various embodiments, the B cells are CD20+ B cells.

In at least one aspect, the present disclosure provides a hematopoietic cell including an adenoviral vector and an adenoviral vector genome, where the adenoviral vector includes a capsid includes one or more viral polypeptides of an Ad3, Ad5, Ad7, Ad11, Ad14, Ad16, Ad21, Ad34, Ad35, Ad37, or Ad50 serotype, the one or more viral polypeptides including one or more of a: (i) fiber knob; (ii) fiber shaft; (iii) fiber tail; (iv) penton; and (v) hexon, where the adenoviral vector genome includes a double-stranded DNA genome including a heterologous nucleic acid payload, and where the hematopoietic cell is an HSC, common lymphoid progenitors (CLPs), T cell, NK cell, colony forming unit (CFU)-pre B cell, B cell, common myeloid progenitor (CMP) cell, granulocyte-macrophage progenitor (GMP) cell, CFU-M cell, monoblasts, monocyte, macrophage, CFU-G cell, myeloblast, granulocyte, neutrophil, eosinophil, basophil, megakaryocyte-erythrocyte progenitor (MEP) cell, BFU-E cell, CFU-E cell, erythroblast, erythrocyte, CFU-Mk cell, megakaryocyte, and/or platelet, optionally where the HSC cell is a CD34⁺ long-term hematopoietic stem cell (LT-HSC) and/or CD34⁺ short term (ST)-HSC.

In at least one aspect, the present disclosure provides a hematopoietic cell including an adenoviral vector genome, where the adenoviral vector genome includes (a) a 3′ ITR and a 5′ ITR, where the 3′ ITR and the 5′ ITR are each of the same serotype selected from Ad3, Ad5, Ad7, Ad11, Ad14, Ad16, Ad21, Ad34, Ad35, Ad37, and Ad50; (b) a packaging sequence, where the packing sequence is of the same serotype as the 3′ ITR and a 5′ ITR; and (c) a heterologous nucleic acid payload, and where the hematopoietic cell is an HSC, common lymphoid progenitors (CLPs), T cell, NK cell, colony forming unit (CFU)-pre B cell, B cell, common myeloid progenitor (CMP) cell, granulocyte-macrophage progenitor (GMP) cell, CFU-M cell, monoblasts, monocyte, macrophage, CFU-G cell, myeloblast, granulocyte, neutrophil, eosinophil, basophil, megakaryocyte-erythrocyte progenitor (MEP) cell, BFU-E cell, CFU-E cell, erythroblast, erythrocyte, CFU-Mk cell, megakaryocyte, and/or platelet, optionally where the HSC cell is a CD34⁺ long-term hematopoietic stem cell (LT-HSC) and/or CD34⁺ short-term (ST)-HSC. In various embodiments, the cell is a cell of a subject suffering from a condition selected from adenosine deaminase deficiency (ADA), adrenoleukodystrophy (ALD), agammaglobulinemia, alpha-1 antitrypsin deficiency, congenital amegakaryocytic thrombocytopenia, amyotrophic lateral sclerosis (Lou Gehrig's disease), ataxia telangiectasia, Batten disease, Bernard-Soulier Syndrome, CD40/CD40L deficiency, chronic granulomatous disease, common variable immune deficiency (CVID), congenital thrombotic thrombocytopenic purpura (cTTP), cystic fibrosis, Diamond Blackfan anemia (DBA), DOCK 8 deficiency, dyskeratosis congenital, Fabry disease, Factor V Deficiency, Factor VII Deficiency, Factor X Deficiency, Factor XI Deficiency, Factor XII Deficiency, Factor XIII Deficiency, familial apolipoprotein E deficiency and atherosclerosis (ApoE), familial erythrophagocytic lymphohistiocytosis, Fanconi anemia (FA), Friedreich ataxia, Gaucher disease, Glanzmann thrombasthenia, glucosemia, glycogen storage disease, glycogen storage disease type I (GSDI), Gray Platelet Syndrome, hemophilia, hemophilia A, hemophilia B, hereditary hemochromatosis, Hurler's syndrome, hyper IgM, Hypogammaglobulinemia, Krabbe disease, major histocompatibility complex class II deficiency (MHC-II), maple syrup urine disease, metachromatic leukodystrophy (MLD), mucopolysaccharidoses, mucopolysaccharidosis type I (MPS I), MPS II (Hunter Syndrome), MPS III (Sanfilippo syndrome), MPS IV (Morquio syndrome), MPS V, MPS VI (Maroteaux-Lamy syndrome), MPS VII (sly syndrome), muscular dystrophy, Niemann-Pick disease, Parkinson's disease, paroxysmal nocturnal hemoglobinuria (PNH), pernicious anemia, phenylketonuria (PKU), Pompe disease, pulmonary alveolar proteinosis (PAP), pure red cell aplasia (PRCA), pyruvate kinase deficiency, refractory anemia, Shwachman-Diamond syndrome, selective IgA deficiency, severe aplastic anemia, severe combined immunodeficiency disease (SCID), Severe combined immunodeficiency due to adenosine deaminase deficiency (ADA-SCID), sickle cell anemia, sickle cell disease, sickle cell trait, Tay Sachs, thalassemia, thalassemia intermedia, von Gierke disease, von Willebrand Disease, Wiskott-Aldrich syndrome (WAS), X-linked agammaglobulinemia (XLA), X-linked severe combined immunodeficiency (SCID-X1), Zellweger syndrome, α-mannosidosis, β-mannosidosis, β-thalassemia, and/or β-thalassemia major.

In at least one aspect, the present disclosure provides a method of in vivo gene therapy in a mammalian subject, the method including administering to the subject an adenoviral vector, where the adenoviral vector includes: (a) a capsid including one or more viral polypeptides of an Ad3, Ad7, Ad11, Ad14, Ad16, Ad21, Ad34, Ad37, or Ad50 serotype, where the one or more viral polypeptides include one or more of a: (i) fiber knob; (ii) fiber shaft; (iii) fiber tail; (iv) penton; and (v) hexon; and (b) a double-stranded DNA genome including a heterologous nucleic acid payload. In various embodiments, the genome further includes: (a) a 3′ ITR and a 5′ ITR, where each of the 3′ ITR and the 5′ ITR are of the viral polypeptide serotype; and (b) a packaging sequence, where the packing sequence is of the viral polypeptide serotype. In various embodiments, the method includes mobilization of hematopoietic stem cells of the subject prior to administration of the adenoviral vector. In various embodiments, the heterologous nucleic acid payload includes a selectable marker, optionally where the selectable marker is MGMT^{P140K}. In various embodiments, the method includes administering a selecting agent to the subject, optionally where the selecting agent includes O⁶BG and/or BCNU. In various embodiments, the method includes administering one or more immunosuppression agents to the subject, optionally where the administration of the one or more immunosuppression agents is prior to the administration of the adenoviral vector.

In at least one aspect, the present disclosure provides an adenoviral donor vector including: (a) a capsid including one or more viral polypeptides of an Ad3, Ad7, Ad11, Ad14, Ad16, Ad21, Ad34, Ad37, or Ad50 serotype, where the one or more viral polypeptides include one or more of a: (i) fiber knob; (ii) fiber shaft; (iii) fiber tail; (iv) penton; and (v) hexon; and (b) a double-stranded DNA genome including a heterologous nucleic acid payload. In various embodiments, the genome further includes: (a) a 3′ ITR and a 5′ ITR, where each of the 3′ ITR and the 5′ ITR are of the viral polypeptide serotype; and (b) a packaging sequence, where the packing sequence is of the viral polypeptide serotype. In various embodiments, the heterologous nucleic acid payload includes a selectable marker, optionally where the selectable marker is MGMT^{P140K}.

In various embodiments, the one or more viral polypeptides include the: (a) fiber knob and fiber shaft; (b) fiber knob and fiber tail; (c) fiber knob and penton; (d) fiber knob and hexon; (e) fiber knob, hexon, and penton; (f) fiber shaft and fiber tail; (g) fiber shaft and penton; (h) fiber shaft and hexon; (i) fiber shaft, hexon, and penton; (j) fiber tail and penton; (k) fiber tail and hexon; (l) fiber tail, hexon, and penton; (m) fiber knob, fiber shaft, and fiber tail; (n) fiber knob, fiber shaft, and penton; (o) fiber knob, fiber shaft, and hexon; (p) fiber knob, fiber shaft, hexon, and penton; (q) fiber knob, fiber shaft, fiber tail, and penton; (r) fiber knob, fiber shaft, fiber tail, penton, and hexon; or (s) penton and hexon. In various embodiments, the fiber knob has a sequence that has at least 80% identity to a sequence selected from SEQ ID NOs: 15, 33, 51, 69, 87, 105, 123, 141, and 159. In various embodiments, the fiber shaft has a sequence that has at least 80% identity to a sequence selected from SEQ ID NOs: 14, 32, 50, 68, 86, 104, 122, 140, and 158. In various embodiments, the fiber tail has a sequence that has at least 80% identity to a sequence selected from SEQ ID NOs: 18, 36, 54, 72, 90, 108, 126, 144, and 162. In various embodiments, the penton has a sequence that has at least 80% identity to a sequence selected from SEQ ID NOs: 16, 34, 52, 70, 88, 106, 124, 142, and 160. In various embodiments, the hexon has a sequence that has at least 80% identity to a sequence selected from SEQ ID NOs: 17, 35, 53, 71, 89, 107, 125, 143, and 161. In various embodiments, the adenoviral vector includes a fiber of the serotype of the viral peptides. In various embodiments, the fiber has a sequence that has at least 80% identity to a sequence selected from SEQ ID NOs: SEQ ID NOs: 13, 31, 49, 67, 85, 103, 121, 139, and 157. In various embodiments, the adenoviral vector is a chimeric vector characterized in that the capsid includes at least one of a fiber knob, fiber shaft, fiber tail, hexon, or penton that is not of the serotype of the viral peptides. In various embodiments, the adenoviral vector is a helper dependent vector.

In at least one aspect, the present disclosure provides an adenoviral donor vector genome including: (a) a 3′ ITR and a 5′ ITR, where the 3′ ITR and the 5′ ITR are each of the same serotype selected from Ad3, Ad7, Ad11, Ad14, Ad16, Ad21, Ad34, Ad37, and Ad50; (b) a packaging sequence, where the packing sequence is of the ITR serotype; and (c) a heterologous nucleic acid payload. In various embodiments, the heterologous nucleic acid payload includes a selectable marker, optionally where the selectable marker is MGMT^{P140K}.

In various embodiments, the heterologous nucleic acid payload encodes a protein. In various embodiments, the heterologous nucleic acid payload encodes a chimeric antigen receptor (CAR), T cell receptor (TCR), or small RNA, optionally where the small RNA is an shRNA. In various embodiments, the heterologous nucleic acid payload encodes a gene editing enzyme or system, where the gene editing is selected from CRISPR editing, base editing, prime editing, or zinc finger nuclease editing. In various embodiments, the heterologous nucleic acid payload encodes an agent for treatment of a condition selected from adenosine deaminase deficiency (ADA), adrenoleukodystrophy (ALD), agammaglobulinemia, alpha-1 antitrypsin deficiency, congenital amegakaryocytic thrombocytopenia, amyotrophic lateral sclerosis (Lou Gehrig's disease), ataxia telangiectasia, Batten disease, Bernard-Soulier Syndrome, CD40/CD40L deficiency, chronic granulomatous disease, common variable immune deficiency (CVID), congenital thrombotic thrombocytopenic purpura (cTTP), cystic fibrosis, Diamond Blackfan anemia (DBA), DOCK 8 deficiency, dyskeratosis congenital, Fabry disease, Factor V Deficiency, Factor VII Deficiency, Factor X Deficiency, Factor XI Deficiency, Factor XII Deficiency, Factor XIII Deficiency, familial apolipoprotein E deficiency and atherosclerosis (ApoE), familial erythrophagocytic lymphohistiocytosis, Fanconi anemia (FA), Friedreich ataxia, Gaucher disease, Glanzmann thrombasthenia, glucosemia, glycogen storage disease, glycogen storage disease type I (GSDI), Gray Platelet Syndrome, hemophilia, hemophilia A, hemophilia B, hereditary hemochromatosis, Hurler's syndrome, hyper IgM, Hypogammaglobulinemia, Krabbe disease, major histocompatibility complex class II deficiency (MHC-II), maple syrup urine disease, metachromatic leukodystrophy (MLD), mucopolysaccharidoses, mucopolysaccharidosis type I (MPS I), MPS II (Hunter Syndrome), MPS III (Sanfilippo syndrome), MPS IV (Morquio syndrome), MPS V, MPS VI (Maroteaux-Lamy syndrome), MPS VII (sly syndrome), muscular dystrophy, Niemann-Pick disease, Parkinson's disease, paroxysmal nocturnal hemoglobinuria (PNH), pernicious anemia, phenylketonuria (PKU), Pompe disease, pulmonary alveolar proteinosis (PAP), pure red cell aplasia (PRCA), pyruvate kinase deficiency, refractory anemia, Shwachman-Diamond syndrome, selective IgA deficiency, severe aplastic anemia, severe combined immunodeficiency disease (SCID), Severe combined immunodeficiency due to adenosine deaminase deficiency (ADA-SCID), sickle cell anemia, sickle cell disease, sickle cell trait, Tay Sachs, thalassemia, thalassemia intermedia, von Gierke disease, von Willebrand Disease, Wiskott-Aldrich syndrome (WAS), X-linked agammaglobulinemia (XLA), X-linked severe combined immunodeficiency (SCID-X1), Zellweger syndrome, α-mannosidosis, β-mannosidosis, β-thalassemia, and/or β-thalassemia major.

In various embodiments, the present disclosure provides a pharmaceutical composition including an adenoviral vector of the present disclosure, where the pharmaceutical composition is formulated for injection to a subject in need thereof.

### Definitions

A, An, The: As used herein, “a”, “an”, and “the” refer to one or to more than one (i.e., to at least one) of the grammatical object of the article. By way of example, “an element” discloses embodiments of exactly one element and embodiments including more than one element.

About: As used herein, term “about”, when used in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referenced value.

Administration: As used herein, the term “administration” typically refers to administration of a composition to a subject or system to achieve delivery of an agent that is, or is included in, the composition.

Adoptive cell therapy: As used herein, “adoptive cell therapy” or “ACT” involves transfer of cells with a therapeutic activity into a subject, e.g., a subject in need of treatment for a condition, disorder, or disease. In some embodiments, ACT includes transfer into a subject of cells after ex vivo and/or in vitro engineering and/or expansion of the cells.

Affinity: As used herein, “affinity” refers to the strength of the sum total of non-covalent interactions between a particular binding agent (e.g., a viral vector), and/or a binding moiety thereof, with a binding target (e.g., a cell or cell type). Unless indicated otherwise, as used herein, “binding affinity” refers to a 1:1 interaction between a binding agent and a binding target thereof (e.g., a viral vector with a target cell of the viral vector). Those of skill in the art appreciate that a change in affinity can be described by comparison to a reference (e.g., increased or decreased relative to a reference), or can be described numerically. Affinity can be measured and/or expressed in a number of ways known in the art, including, but not limited to, equilibrium dissociation constant (K_{D}) and/or equilibrium association constant (K_{A}). K_{D} is the quotient of k_{off}/kₒₙ, whereas K_{A} is the quotient of kₒₙ/k_{off}, where kₒₙ refers to the association rate constant of, e.g., viral vector with target cell, and k_{off} refers to the dissociation of, e.g., viral vector from target cell. The kₒₙ and k_{off} can be determined by techniques known to those of skill in the art.

Agent: As used herein, the term “agent” may refer to any chemical entity, including without limitation any of one or more of an atom, molecule, compound, amino acid, polypeptide, nucleotide, nucleic acid, protein, protein complex, liquid, solution, saccharide, polysaccharide, lipid, or combination or complex thereof.

Allogeneic: As used herein, term “allogeneic” refers to any material derived from one subject which is then introduced to another subject, e.g., allogeneic HSC transplantation.

Antibody: As used herein, the term “antibody” refers to a polypeptide that includes one or more canonical immunoglobulin sequence elements sufficient to confer specific binding to a particular antigen (e.g., a heavy chain variable domain, a light chain variable domain, and/or one or more CDRs). Thus, the term antibody includes, without limitation, human antibodies, non-human antibodies, synthetic and/or engineered antibodies, fragments thereof, and agents including the same. Antibodies can be naturally occurring immunoglobulins (e.g., generated by an organism reacting to an antigen). Synthetic, non-naturally occurring, or engineered antibodies can be produced by recombinant engineering, chemical synthesis, or other artificial systems or methodologies known to those of skill in the art.

As is well known in the art, typical human immunoglobulins are approximately 150 kD tetrameric agents that include two identical heavy (H) chain polypeptides (about 50 kD each) and two identical light (L) chain polypeptides (about 25 kD each) that associate with each other to form a structure commonly referred to as a “Y-shaped” structure. Typically, each heavy chain includes a heavy chain variable domain (VH) and a heavy chain constant domain (CH). The heavy chain constant domain includes three CH domains: CH1, CH2 and CH3. A short region, known as the “switch”, connects the heavy chain variable and constant regions. The “hinge” connects CH2 and CH3 domains to the rest of the immunoglobulin. Each light chain includes a light chain variable domain (VL) and a light chain constant domain (CL), separated from one another by another “switch.” Each variable domain contains three hypervariable loops known as “complement determining regions” (CDR1, CDR2, and CDR3) and four somewhat invariant “framework” regions (FR1, FR2, FR3, and FR4). In each VH and VL, the three CDRs and four FRs are arranged from amino-terminus to carboxy-terminus in the following order: FR1, CDR1, FR2, CDR2, FR3, CDR3, and FR4. The variable regions of a heavy and/or a light chain are typically understood to provide a binding moiety that can interact with an antigen. Constant domains can mediate binding of an antibody to various immune system cells (e.g., effector cells and/or cells that mediate cytotoxicity), receptors, and elements of the complement system. Heavy and light chains can be linked to one another by a single disulfide bond, and two other disulfide bonds can connect the heavy chain hinge regions to one another, so that dimers are connected to one another and the tetramer is formed. When natural immunoglobulins fold, the FR regions form the beta sheets that provide the structural framework for the domains, and the CDR loop regions from both the heavy and light chains are brought together in three-dimensional space so that they create a single hypervariable antigen binding site located at the tip of the Y structure.

In some embodiments, an antibody is a polyclonal, monoclonal, monospecific, or multispecific antibody (e.g., a bispecific antibody). In some embodiments, an antibody includes at least one light chain monomer or dimer, at least one heavy chain monomer or dimer, at least one heavy chain-light chain dimer, or a tetramer that includes two heavy chain monomers and two light chain monomers. Moreover, the term “antibody” can include (unless otherwise stated or clear from context) any art-known constructs or formats utilizing antibody structural and/or functional features including without limitation intrabodies, domain antibodies, antibody mimetics, Zybodies®, Fab fragments, Fab′ fragments, F(ab′)2 fragments, Fd′ fragments, Fd fragments, isolated CDRs or sets thereof, single chain antibodies, single-chain Fvs (scFvs), disulfide-linked Fvs (sdFv), polypeptide-Fc fusions, single domain antibodies (e.g., shark single domain antibodies such as IgNAR or fragments thereof), cameloid antibodies, camelized antibodies, masked antibodies (e.g., Probodies®), affybodies, anti-idiotypic (anti-Id) antibodies (including, e.g., anti-anti-Id antibodies), Small Modular ImmunoPharmaceuticals (“SMIPs™”), single chain or Tandem diabodies (TandAb®), VHHs, Anticalins®, Nanobodies® minibodies, BiTE®s, ankyrin repeat proteins or DARPINs®, Avimers®, DARTs, TCR-like antibodies, Adnectins®, Affilins®, Trans-bodies®, Affibodies®, TrimerX®, MicroProteins, Fynomers®, Centyrins®, and KALBITOR®s, CARs, engineered TCRs, and antigen-binding fragments of any of the above.

In various embodiments, an antibody includes one or more structural elements recognized by those skilled in the art as a complementarity determining region (CDR) or variable domain. In some embodiments, an antibody can be a covalently modified (“conjugated”) antibody (e.g., an antibody that includes a polypeptide including one or more canonical immunoglobulin sequence elements sufficient to confer specific binding to a particular antigen, where the polypeptide is covalently linked with one or more of a therapeutic agent, a detectable moiety, another polypeptide, a glycan, or a polyethylene glycol molecule). In some embodiments, antibody sequence elements are humanized, primatized, chimeric, etc, as is known in the art.

An antibody including a heavy chain constant domain can be, without limitation, an antibody of any known class, including but not limited to, IgA, secretory IgA, IgG, IgE and IgM, based on heavy chain constant domain amino acid sequence (e.g., alpha (α), delta (δ), epsilon (ε), gamma (γ) and mu (μ)). IgG subclasses are also well known to those in the art and include but are not limited to human IgG1, IgG2, IgG3 and IgG4. “Isotype” refers to the Ab class or subclass (e.g., IgM or IgG1) that is encoded by the heavy chain constant region genes. As used herein, a “light chain” can be of a distinct type, e.g., kappa (κ) or lambda (λ), based on the amino acid sequence of the light chain constant domain. In some embodiments, an antibody has constant region sequences that are characteristic of mouse, rabbit, primate, or human immunoglobulins. Naturally-produced immunoglobulins are glycosylated, typically on the CH2 domain. As is known in the art, affinity and/or other binding attributes of Fc regions for Fc receptors can be modulated through glycosylation or other modification. In some embodiments, an antibody may lack a covalent modification (e.g., attachment of a glycan) that it would have if produced naturally. In some embodiments, antibodies produced and/or utilized in accordance with the present invention include glycosylated Fc domains, including Fc domains with modified or engineered such glycosylation.

Between or From: As used herein, the term “between” refers to content that falls between indicated upper and lower, or first and second, boundaries, inclusive of the boundaries. Similarly, the term “from”, when used in the context of a range of values, indicates that the range includes content that falls between indicated upper and lower, or first and second, boundaries, inclusive of the boundaries.

Binding: As used herein, the term “binding” refers to a non-covalent association between or among two or more agents. “Direct” binding involves physical contact between agents; indirect binding involves physical interaction by way of physical contact with one or more intermediate agents. Binding between two or more agents can occur and/or be assessed in any of a variety of contexts, including where interacting agents are studied in isolation or in the context of more complex systems (e.g., while covalently or otherwise associated with a carrier agents and/or in a biological system or cell).

Biological Sample: As used herein, the term “biological sample” refers to a sample obtained or derived from a biological source (e.g., a tissue or organism or cell culture) of interest, as described herein. In some embodiments, a biological source is or includes an organism, such as an animal or human. In some embodiments, a biological sample is or includes biological tissue or fluid. In some embodiments, a biological sample can be or include cells (e.g., hematopoietic cells), tissue, or bodily fluid (e.g., blood). A biological sample can be a “primary sample” obtained directly from a biological source, or can be a “processed sample” (e.g., a sample prepared from a primary sample). A biological sample can also be referred to as a “sample.”

Cancer: As used herein, the term “cancer” refers to a condition, disorder, or disease in which cells exhibit relatively abnormal, uncontrolled, and/or autonomous growth, so that they display an abnormally elevated proliferation rate and/or aberrant growth phenotype characterized by a significant loss of control of cell proliferation. In some embodiments, a cancer can include one or more tumors. In some embodiments, a cancer can be or include cells that are precancerous (e.g., benign), malignant, pre-metastatic, metastatic, and/or non-metastatic. In some embodiments, a cancer can be or include a solid tumor. In some embodiments, a cancer can be or include a hematologic tumor.

Chimeric antigen receptor: As used herein, “Chimeric antigen receptor” or “CAR” refers to an engineered protein that includes (i) an extracellular domain that includes a moiety that binds a target antigen; (ii) a transmembrane domain; and (iii) an intracellular signaling domain that sends activating signals when the CAR is stimulated by binding of the extracellular binding moiety with a target antigen. CARs are also known as chimeric T cell receptors or chimeric immunoreceptors.

Combination therapy: As used herein, the term “combination therapy” refers to administration to a subject of to two or more agents or regimens such that the two or more agents or regimens together treat a condition, disorder, or disease of the subject. In some embodiments, the two or more therapeutic agents or regimens can be administered simultaneously, sequentially, or in overlapping dosing regimens. Those of skill in the art will appreciate that combination therapy includes but does not require that the two agents or regimens be administered together in a single composition, nor at the same time.

Control expression or activity: As used herein, a first element (e.g., a protein, such as a transcription factor, or a nucleic acid sequence, such as promoter) “controls” or “drives” expression or activity of a second element (e.g., a protein or a nucleic acid encoding an agent such as a protein) if the expression or activity of the second element is wholly or partially dependent upon status (e.g., presence, absence, conformation, chemical modification, interaction, or other activity) of the first under at least one set of conditions. Control of expression or activity can be substantial control or activity, e.g., in that a change in status of the first element can, under at least one set of conditions, result in a change in expression or activity of the second element of at least 10% (e.g., at least 20%, 30%, 40%, 50%, 60%, 70%, 80%, 90%, 100%, 2-fold, 3-fold, 4-fold, 5-fold, 10-fold, 20-fold, 30-fold, 40-fold, 50-fold, 100-fold) as compared to a reference control.

Corresponding to: As used herein, the term “corresponding to” may be used to designate the position/identity of a structural element in a compound or composition through comparison with an appropriate reference compound or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of skill in the art appreciate that residues in a provided polypeptide or polynucleotide sequence are often designated (e.g., numbered or labeled) according to the scheme of a related reference sequence (even if, e.g., such designation does not reflect literal numbering of the provided sequence). By way of illustration, if a reference sequence includes a particular amino acid motif at positions 100-110, and a second related sequence includes the same motif at positions 110-120, the motif positions of the second related sequence can be said to “correspond to” positions 100-110 of the reference sequence. Those of skill in the art appreciate that corresponding positions can be readily identified, e.g., by alignment of sequences, and that such alignment is commonly accomplished by any of a variety of known tools, strategies, and/or algorithms, including without limitation software programs such as, for example, BLAST, CS-BLAST, CUDASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE.

Dosing regimen: As used herein, the term “dosing regimen” can refer to a set of one or more same or different unit doses administered to a subject, typically including a plurality of unit doses administration of each of which is separated from administration of the others by a period of time. In various embodiments, one or more or all unit doses of a dosing regimen may be the same or can vary (e.g., increase over time, decrease over time, or be adjusted in accordance with the subject and/or with a medical practitioner's determination). In various embodiments, one or more or all of the periods of time between each dose may be the same or can vary (e.g., increase over time, decrease over time, or be adjusted in accordance with the subject and/or with a medical practitioner's determination). In some embodiments, a given therapeutic agent has a recommended dosing regimen, which can involve one or more doses. Typically, at least one recommended dosing regimen of a marketed drug is known to those of skill in the art. In some embodiments, a dosing regimen is correlated with a desired or beneficial outcome when administered across a relevant population (i.e., is a therapeutic dosing regimen).

Downstream and Upstream: As used herein, the term“downstream” means that a first DNA region is closer, relative to a second DNA region, to the C-terminus of a nucleic acid that includes the first DNA region and the second DNA region. As used herein, the term “upstream” means a first DNA region is closer, relative to a second DNA region, to the N-terminus of a nucleic acid that includes the first DNA region and the second DNA region.

Effective amount: An “effective amount” is the amount of a composition (e.g., a formulation) necessary to result in a desired physiological change in a subject. Effective amounts are often administered for research purposes.

Engineered: As used herein, the term “engineered” refers to the aspect of having been manipulated by the hand of man. For example, a polynucleotide is considered to be “engineered” when two or more sequences, that are not linked together in that order in nature, are manipulated by the hand of man to be directly linked to one another in the engineered polynucleotide. Those of skill in the art will appreciate that an “engineered” nucleic acid or amino acid sequence can be a recombinant nucleic acid or amino acid sequence, and can be referred to as “genetically engineered.” In some embodiments, an engineered polynucleotide includes a coding sequence and/or a regulatory sequence that is found in nature operably linked with a first sequence but is not found in nature operably linked with a second sequence, which is in the engineered polynucleotide operably linked in with the second sequence by the hand of man. In some embodiments, a cell or organism is considered to be “engineered” or “genetically engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution, deletion, or mating). As is common practice and is understood by those of skill in the art, progeny or copies, perfect or imperfect, of an engineered polynucleotide or cell are typically still referred to as “engineered” even though the direct manipulation was of a prior entity.

Excipient: As used herein, “excipient” refers to a non-therapeutic agent that may be included in a pharmaceutical composition, for example to provide or contribute to a desired consistency or stabilizing effect. In some embodiments, suitable pharmaceutical excipients may include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, or the like.

Expression: As used herein, “expression” refers individually and/or cumulatively to one or more biological process that result in production from a nucleic acid sequence of an encoded agent, such as a protein. Expression specifically includes either or both of transcription and translation.

Flank: As used herein, a first element (e.g., a nucleic acid sequence or amino acid sequence) present in a contiguous sequence with a second element and a third element is “flanked” by the second element and third element if it is positioned in the contiguous sequence between the second element and the third element. Accordingly, in such arrangement, the second element and third element can be referred to as “flanking” the first element. Flanking elements can be immediately adjacent to a flanked element or separated from the flanked element by one or more relevant units. In various examples in which the contiguous sequence is a nucleic acid or amino acid sequence, and the relevant units are bases or amino acid residues, respectively, the number of units in the contiguous sequence that are between a flanked element and, independently, first and/or second flanking elements can be, e.g., 50 units or less, e.g., no more than 50, 45, 40, 35, 30, 25, 20, 15, 10, 5, 4, 3, 2, 1, or 0 units.

Fragment: As used herein, “fragment” refers a structure that includes and/or consists of a discrete portion of a reference agent (sometimes referred to as the “parent” agent). In some embodiments, a fragment lacks one or more moieties found in the reference agent. In some embodiments, a fragment includes or consists of one or more moieties found in the reference agent. In some embodiments, the reference agent is a polymer such as a polynucleotide or polypeptide. In some embodiments, a fragment of a polymer includes or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) of the reference polymer. In some embodiments, a fragment of a polymer includes or consists of at least 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the reference polymer. A fragment of a reference polymer is not necessarily identical to a corresponding portion of the reference polymer. For example, a fragment of a reference polymer can be a polymer having a sequence of residues having at least 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more identity to the reference polymer. A fragment may, or may not, be generated by physical fragmentation of a reference agent. In some instances, a fragment is generated by physical fragmentation of a reference agent. In some instances, a fragment is not generated by physical fragmentation of a reference agent and can be instead, for example, produced by de novo synthesis or other means.

Gene, Transgene: As used herein, the term “gene” refers to a DNA sequence that is or includes coding sequence (i.e., a DNA sequence that encodes an expression product, such as an RNA product and/or a polypeptide product), optionally together with some or all of regulatory sequences that control expression of the coding sequence. In some embodiments, a gene includes non-coding sequence such as, without limitation, introns. In some embodiments, a gene may include both coding (e.g., exonic) and non-coding (e.g., intronic) sequences. In some embodiments, a gene includes a regulatory sequence that is a promoter. In some embodiments, a gene includes one or both of a (i) DNA nucleotides extending a predetermined number of nucleotides upstream of the coding sequence in a reference context, such as a source genome, and (ii) DNA nucleotides extending a predetermined number of nucleotides downstream of the coding sequence in a reference context, such as a source genome. In various embodiments, the predetermined number of nucleotides can be 500 bp, 1 kb, 2 kb, 3 kb, 4 kb, 5 kb, 10 kb, 20 kb, 30 kb, 40 kb, 50 kb, 75 kb, or 100 kb. As used herein, a “transgene” refers to a gene that is not endogenous or native to a reference context in which the gene is present or into which the gene may be placed by engineering.

Gene product or expression product: As used herein, the term “gene product” or “expression product” generally refers to an RNA transcribed from the gene (pre-and/or post-processing) or a polypeptide (pre- and/or post-modification) encoded by an RNA transcribed from the gene.

Host cell, target cell: As used herein, “host cell” refers to a cell into which exogenous DNA (recombinant or otherwise), such as a transgene, has been introduced. Those of skill in the art appreciate that a “host cell” can be the cell into which the exogenous DNA was initially introduced and/or progeny or copies, perfect or imperfect, thereof. In some embodiments, a host cell includes one or more viral genes or transgenes. In some embodiments, a host cell is a cell that has been entered by a viral vector, e.g., a vector of the present disclosure, or a viral genome thereof, e.g., a viral genome disclosed herein. In some embodiments, an intended or potential host cell can be referred to as a target cell. In some embodiments, a cell or type of cell that is selectively entered and/or selectively transduced by a viral vector of the present disclosure can be referred to as a target cell of the viral vector. In some embodiments, a host cell that has been entered and/or transduced (e.g., selectively entered and/or selectively transduced) by a viral vector of the present disclosure can be referred to as a target cell of the viral vector. In some embodiments, the terms “host cell” or “target cell” include progeny of a cell that has been entered and/or transduced (e.g., selectively entered and/or selectively transduced) by a viral vector of the present disclosure, e.g., progeny that include exogenous DNA sequences derived from DNA sequences introduced by the viral vector.

In various embodiments, a host cell or target cell is identified by the presence, absence, or expression level of various surface markers.

A statement that a cell or population of cells is “positive” for or expressing a particular marker refers to the detectable presence on or in the cell of the particular marker. When referring to a surface marker, the term can refer to the presence of surface expression as detected by flow cytometry, for example, by staining with an antibody that specifically binds to the marker and detecting said antibody, where the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

A statement that a cell or population of cells is “negative” for a particular marker or lacks expression of a marker refers to the absence of substantial detectable presence on or in the cell of a particular marker. When referring to a surface marker, the term can refer to the absence of surface expression as detected by flow cytometry, for example, by staining with an antibody that specifically binds to the marker and detecting said antibody, where the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

Identity: As used herein, the term “identity” refers to the overall relatedness between polymeric molecules, e.g., between nucleic acid molecules (e.g., DNA molecules and/or RNA molecules) and/or between polypeptide molecules. Methods for the calculation of a percent identity as between two provided sequences are known in the art. The term “% sequence identity” refers to a relationship between two or more sequences, as determined by comparing the sequences. In the art, “identity” also means the degree of sequence relatedness between protein and nucleic acid sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. For instance, calculation of the percent identity of two nucleic acid or polypeptide sequences, for example, can be performed by aligning the two sequences (or the complement of one or both sequences) for optimal comparison purposes (e.g., gaps can be introduced in one or both of a first and a second sequences for optimal alignment and non-identical sequences can be disregarded for comparison purposes). The nucleotides or amino acids at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, optionally accounting for the number of gaps, and the length of each gap, which may need to be introduced for optimal alignment of the two sequences. The comparison of sequences and determination of percent identity between two sequences can be accomplished using a computational algorithm, such as BLAST (basic local alignment search tool). Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.](1994), Meeting Date 1992, 111-20. Editor(s): Suhai, Sandor. Publisher: Plenum, New York, N.Y. Within the context of this disclosure, it will be understood that where sequence analysis software is used for analysis, the results of the analysis are based on the “default values” of the program referenced. “Default values” will mean any set of values or parameters, which originally load with the software when first initialized.

“Improve,” “increase,” “inhibit,” or “reduce”: As used herein, the terms “improve”, “increase”, “inhibit”, and “reduce”, and grammatical equivalents thereof, indicate qualitative or quantitative difference from a reference.

Isolated: As used herein, “isolated” refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from 10%, 20%, 30%, 40%, 50%, 60%, 70%, 80%, 90%, 91%, 92%, 93%, 94%, 95%, 96%, 97%, 98%, 99%, or more than 99% of the other components with which they were initially associated. In some embodiments, isolated agents are 80%, 85%, 90%, 91%, 92%, 93%, 94%, 95%, 96%, 97%, 98%, 99%, or more than 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.

Operably linked: As used herein, “operably linked” or “operatively linked” refers to the association of at least a first element and a second element such that the component elements are in a relationship permitting them to function in their intended manner. For example, a nucleic acid regulatory sequence is “operably linked” to a nucleic acid coding sequence if the regulatory sequence and coding sequence are associated in a manner that permits control of expression of the coding sequence by the regulatory sequence. In some embodiments, an “operably linked” regulatory sequence is directly or indirectly covalently associated with a coding sequence (e.g., in a single nucleic acid). In some embodiments, a regulatory sequence controls expression of a coding sequence in trans and inclusion of the regulatory sequence in the same nucleic acid as the coding sequence is not a requirement of operable linkage.

Pharmaceutically acceptable: As used herein, the term “pharmaceutically acceptable,” as applied to one or more, or all, component(s) for formulation of a composition as disclosed herein, means that each component must be compatible with the other ingredients of the composition and not deleterious to the recipient thereof.

Pharmaceutically acceptable carrier: As used herein, the term “pharmaceutically acceptable carrier” refers to a pharmaceutically-acceptable material, composition, or vehicle, such as a liquid or solid filler, diluent, excipient, or solvent encapsulating material, that facilitates formulation of an agent (e.g., a pharmaceutical agent), modifies bioavailability of an agent, or facilitates transport of an agent from one organ or portion of a subject to another. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutical composition: As used herein, the term “pharmaceutical composition” refers to a composition in which an active agent is formulated together with one or more pharmaceutically acceptable carriers.

Promoter: As used herein, a “promoter” or “promoter sequence” can be a DNA regulatory region that directly or indirectly (e.g., through promoter-bound proteins or substances) participates in initiation and/or processivity of transcription of a coding sequence. A promoter may, under suitable conditions, initiate transcription of a coding sequence upon binding of one or more transcription factors and/or regulatory moieties with the promoter. A promoter that participates in initiation of transcription of a coding sequence can be “operably linked” to the coding sequence. In certain instances, a promoter can be or include a DNA regulatory region that extends from a transcription initiation site (at its 3′ terminus) to an upstream (5′ direction) position such that the sequence so designated includes one or both of a minimum number of bases or elements necessary to initiate a transcription event. A promoter may be, include, or be operably associated with or operably linked to, expression control sequences such as enhancer and repressor sequences. In some embodiments, a promoter may be inducible. In some embodiments, a promoter may be a constitutive promoter. In some embodiments, a conditional (e.g., inducible) promoter may be unidirectional or bi-directional. A promoter may be or include a sequence identical to a sequence known to occur in the genome of particular species. In some embodiments, a promoter can be or include a hybrid promoter, in which a sequence containing a transcriptional regulatory region can be obtained from one source and a sequence containing a transcription initiation region can be obtained from a second source. Systems for linking control elements to coding sequence within a transgene are well known in the art (general molecular biological and recombinant DNA techniques are described in Sambrook, Fritsch, and Maniatis, Molecular Cloning: A Laboratory Manual, Second Edition, Cold Spring Harbor Laboratory Press, Cold Spring Harbor, NY, 1989).

Reference: As used herein, “reference” refers to a standard or control relative to which a comparison is performed. For example, in some embodiments, an agent, sample, sequence, subject, animal, or individual, or population thereof, or a measure or characteristic representative thereof, is compared with a reference, an agent, sample, sequence, subject, animal, or individual, or population thereof, or a measure or characteristic representative thereof. In some embodiments, a reference is a measured value. In some embodiments, a reference is an established standard or expected value. In some embodiments, a reference is a historical reference. A reference can be quantitative of qualitative. Typically, as would be understood by those of skill in the art, a reference and the value to which it is compared represents measure under comparable conditions. Those of skill in the art will appreciate when sufficient similarities are present to justify reliance on and/or comparison. In some embodiments, an appropriate reference may be an agent, sample, sequence, subject, animal, or individual, or population thereof, under conditions those of skill in the art will recognize as comparable, e.g., for the purpose of assessing one or more particular variables (e.g., presence or absence of an agent or condition), or a measure or characteristic representative thereof. Without wishing to be bound by any particular embodiment(s), in various embodiments a reference sequence can be a sequence associated with a sequence accession number provided herein, certain of which sequences associated with sequence accession numbers are provided in the below listing of accession sequences.

Regulatory sequence: As used herein in the context of expression of a nucleic acid coding sequence, a regulatory sequence is a nucleic acid sequence that controls expression of a coding sequence. In some embodiments, a regulatory sequence can control or impact one or more aspects of gene expression (e.g., cell-type-specific expression, inducible expression, etc.).

Subject: As used herein, the term “subject” refers to an organism, typically a mammal (e.g., a human, rat, or mouse). In some embodiments, a subject is suffering from a disease, disorder or condition. In some embodiments, a subject is susceptible to a disease, disorder, or condition. In some embodiments, a subject displays one or more symptoms or characteristics of a disease, disorder or condition. In some embodiments, a subject is not suffering from a disease, disorder or condition. In some embodiments, a subject does not display any symptom or characteristic of a disease, disorder, or condition. In some embodiments, a subject has one or more features characteristic of susceptibility to or risk of a disease, disorder, or condition. In some embodiments, a subject is a subject that has been tested for a disease, disorder, or condition, and/or to whom therapy has been administered. In some instances, a human subject can be interchangeably referred to as a “patient” or “individual.”

Therapeutic agent: As used herein, the term “therapeutic agent” refers to any agent that elicits a desired pharmacological effect when administered to a subject. In some embodiments, an agent is considered to be a therapeutic agent if it demonstrates a statistically significant effect across an appropriate population. In some embodiments, the appropriate population can be a population of model organisms or a human population. In some embodiments, an appropriate population can be defined by various criteria, such as a certain age group, gender, genetic background, preexisting clinical conditions, etc. In some embodiments, a therapeutic agent is a substance that can be used for treatment of a disease, disorder, or condition. In some embodiments, a therapeutic agent is an agent that has been or is required to be approved by a government agency before it can be marketed for administration to humans. In some embodiments, a therapeutic agent is an agent for which a medical prescription is required for administration to humans.

Therapeutically effective amount: As used herein, “therapeutically effective amount” refers to an amount that produces the desired effect for which it is administered. In some embodiments, the term refers to an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

Treatment: As used herein, the term “treatment” (also “treat” or “treating”) refers to administration of a therapy that partially or completely alleviates, ameliorates, relieves, inhibits, delays onset of, reduces severity of, and/or reduces incidence of one or more symptoms, features, and/or causes of a particular disease, disorder, or condition, or is administered for the purpose of achieving any such result. In some embodiments, such treatment can be of a subject who does not exhibit signs of the relevant disease, disorder, or condition and/or of a subject who exhibits only early signs of the disease, disorder, or condition. Alternatively or additionally, such treatment can be of a subject who exhibits one or more established signs of the relevant disease, disorder and/or condition. In some embodiments, treatment can be of a subject who has been diagnosed as suffering from the relevant disease, disorder, and/or condition. In some embodiments, treatment can be of a subject known to have one or more susceptibility factors that are statistically correlated with increased risk of development of the relevant disease, disorder, or condition. A “prophylactic treatment” includes a treatment administered to a subject who does not display signs or symptoms of a condition to be treated or displays only early signs or symptoms of the condition to be treated such that treatment is administered for the purpose of diminishing, preventing, or decreasing the risk of developing the condition. Thus, a prophylactic treatment functions as a preventative treatment against a condition. A “therapeutic treatment” includes a treatment administered to a subject who displays symptoms or signs of a condition and is administered to the subject for the purpose of reducing the severity or progression of the condition.

Unit dose: As used herein, the term “unit dose” refers to an amount administered as a single dose and/or in a physically discrete unit of a pharmaceutical composition. In many embodiments, a unit dose contains a predetermined quantity of an active agent, for instance a predetermined viral titer (the number of viruses, virions, or viral particles in a given volume). In some embodiments, a unit dose contains an entire single dose of the agent. In some embodiments, more than one unit dose is administered to achieve a total single dose. In some embodiments, administration of multiple unit doses is required, or expected to be required, in order to achieve an intended effect. A unit dose can be, for example, a volume of liquid (e.g., an acceptable carrier) containing a predetermined quantity of one or more therapeutic moieties, a predetermined amount of one or more therapeutic moieties in solid form, a sustained release formulation or drug delivery device containing a predetermined amount of one or more therapeutic moieties, etc. It will be appreciated that a unit dose can be present in a formulation that includes any of a variety of components in addition to the therapeutic moiety(s). For example, acceptable carriers (e.g., pharmaceutically acceptable carriers), diluents, stabilizers, buffers, preservatives, etc., can be included. It will be appreciated by those skilled in the art, in many embodiments, a total appropriate daily dosage of a particular therapeutic agent can include a portion, or a plurality, of unit doses, and can be decided, for example, by a medical practitioner within the scope of sound medical judgment. In some embodiments, the specific effective dose level for any particular subject or organism can depend upon a variety of factors including the disorder being treated and the severity of the disorder; activity of specific active compound employed; specific composition employed; age, body weight, general health, sex, and diet of the subject; time of administration, and rate of excretion of the specific active compound employed; duration of the treatment; drugs and/or additional therapies used in combination or coincidental with specific compound(s) employed, and like factors well known in the medical arts.

### BRIEF DESCRIPTION OF THE DRAWING

FIG. 1 is a chart showing results of anti-hexon staining of CD34+ cells three hours after infection of the cells with indicated adenoviral serotypes. Cells were infected at 5,000 viral particles per cell or 2,000 viral particles per cell. For each tested serotype, the chart includes two replicates of data, each replicate including, in the order shown, results of analysis at 5,000 viral particles per cell and 2,000 viral particles per cell. Data represent infection efficiency.

FIG. 2 is a chart showing results of qPCR analysis of adenoviral DNA in CD34+ cells infected with the indicated adenoviral serotypes. Cells were infected at 5,000 viral particles per cell or 2,000 viral particles per cell. For each tested serotype, the chart includes two replicates of data, each replicate including, in the order shown, results of analysis at 5,000 viral particles per cell and 2,000 viral particles per cell. Data represent relative infection efficiency.

FIG. 3 is a schematic of hematopoietic cell differentiation that includes hematopoietic stem cells, progenitor cells, and terminally differentiated cells.

FIG. 4 is a schematic of a plasmid containing an E1-deleted Ad5 genome including an EGFP reporter construct. The schematic depicts a single vector; however solely for the purposes of presentation, the adenoviral genome is depicted in two sections with the hexon region included in each section to indicate the orientation of the sections with respect to each other.

FIG. 5 is a schematic of a plasmid containing an E1-deleted Ad7 genome including an EGFP reporter construct. The schematic depicts a single vector; however solely for the purposes of presentation, the adenoviral genome is depicted in two sections with the hexon region included in each section to indicate the orientation of the sections with respect to each other.

FIG. 6 is a schematic of a plasmid containing an E1-deleted Ad11 genome including an EGFP reporter construct. The schematic depicts a single vector; however solely for the purposes of presentation, the adenoviral genome is depicted in two sections with the hexon region included in each section to indicate the orientation of the sections with respect to each other.

FIG. 7 is a schematic of a plasmid containing an E1-deleted Ad16 genome including an EGFP reporter construct. The schematic depicts a single vector; however solely for the purposes of presentation, the adenoviral genome is depicted in two sections with the hexon region included in each section to indicate the orientation of the sections with respect to each other.

FIG. 8 is a schematic of a plasmid containing an E1-deleted Ad34 genome including an EGFP reporter construct. The schematic depicts a single vector; however solely for the purposes of presentation, the adenoviral genome is depicted in two sections with the hexon region included in each section to indicate the orientation of the sections with respect to each other.

FIG. 9 is a schematic of a plasmid containing an E1-deleted Ad35 genome including an EGFP reporter construct. The schematic depicts a single vector; however solely for the purposes of presentation, the adenoviral genome is depicted in two sections with the hexon region included in each section to indicate the orientation of the sections with respect to each other.

FIG. 10 is a schematic of a plasmid containing an E1-deleted Ad35++ genome including an EGFP reporter construct. The schematic depicts a single vector; however solely for the purposes of presentation, the adenoviral genome is depicted in two sections with the hexon region included in each section to indicate the orientation of the sections with respect to each other.

FIG. 11 is an exemplary depiction of gating used for analysis of monocyte, T cell, NK cell, and B cell populations present in PBMCs using flow cytometry. Boxes indicate gates used to define cell types. Arrows from one plot to another indicate that the gated population in the first plot is being displayed in the second plot. The representative data shown in this figure corresponds to human PBMCs, 48 hours after infection with an E1-deleted adenoviral vector of the present disclosure at an MOI of 2000 viral particles per cell.

FIG. 12 is a chart showing results of GFP analysis of monocytes present in human PBMCs from Donor 1, 48 hours after infection of the cells with E1-deleted adenoviral vectors of the indicated adenoviral serotypes. Monocytes were identified as CD14+/CD11b+ myeloid cells. Percent of cells that are GFP positive is shown. Cells were infected at an MOI of 500, 2000, and 5000 viral particles per cell. At each MOI, data is shown for Ad5, Ad7, Ad11, Ad34, Ad35, Ad35++, and Ad16 (from left to right). Data represents infection efficiency.

FIG. 13 is a chart showing results of GFP analysis of T cells present in human PBMCs from Donor 1, 48 hours after infection of the cells with E1-deleted adenoviral vectors of the indicated adenoviral serotypes. T cells were identified as CD3+ lymphoid cells. Percent of cells that are GFP positive is shown. Cells were infected at an MOI of 500, 2000, and 5000 viral particles per cell. At each MOI, data is shown for Ad5, Ad7, Ad11, Ad34, Ad35, Ad35++, and Ad16 (from left to right). Data represents infection efficiency.

FIG. 14 is a chart showing results of GFP analysis of NK cells present in human PBMCs from Donor 1, 48 hours after infection of the cells with E1-deleted adenoviral vectors of the indicated adenoviral serotypes. NK cells were identified as CD3−/CD56+ lymphoid cells. Percent of cells that are GFP positive is shown. Cells were infected at an MOI of 500, 2000, and 5000 viral particles per cell. At each MOI, data is shown for Ad5, Ad7, Ad11, Ad34, Ad35, Ad35++, and Ad16 (from left to right). Data represents infection efficiency.

FIG. 15 is a chart showing results of GFP analysis of B cells present in human PBMCs from Donor 1, 48 hours after infection of the cells with E1-deleted adenoviral vectors of the indicated adenoviral serotypes. B cells were identified as CD20+ lymphoid cells. Percent of cells that are GFP positive is shown. Cells were infected at an MOI of 500, 2000, and 5000 viral particles per cell. At each MOI, data is shown for Ad5, Ad7, Ad11, Ad34, Ad35, Ad35++, and Ad16 (from left to right). Data represents infection efficiency.

FIG. 16 is a chart showing results of GFP analysis of monocytes present in human PBMCs from Donor 2, 48 hours after infection of the cells with E1-deleted adenoviral vectors of the indicated adenoviral serotypes. Monocytes were identified as CD14+/CD11b+ myeloid cells. Percent of cells that are GFP positive is shown. Cells were infected at an MOI of 500, 2000, and 5000 viral particles per cell. At each MOI, data is shown for Ad5, Ad7, Ad11, Ad34, Ad35, and Ad35++ (from left to right). Data represents infection efficiency.

FIG. 17 is a chart showing results of GFP analysis of T cells present in human PBMCs from Donor 2, 48 hours after infection of the cells with E1-deleted adenoviral vectors of the indicated adenoviral serotypes. T cells were identified as CD3+ lymphoid cells. Percent of cells that are GFP positive is shown. Cells were infected at an MOI of 500, 2000, and 5000 viral particles per cell. At each MOI, data is shown for Ad5, Ad7, Ad1, Ad34, Ad35, and Ad35++ (from left to right). Data represents infection efficiency.

FIG. 18 is a chart showing results of GFP analysis of NK cells present in human PBMCs from Donor 2, 48 hours after infection of the cells with E1-deleted adenoviral vectors of the indicated adenoviral serotypes. NK cells were identified as CD3−/CD56+ lymphoid cells. Percent of cells that are GFP positive is shown. Cells were infected at an MOI of 500, 2000, and 5000 viral particles per cell. At each MOI, data is shown for Ad5, Ad7, Ad11, Ad34, Ad35, and Ad35++ (from left to right). Data represents infection efficiency.

FIG. 19 is a chart showing results of GFP analysis of B cells present in human PBMCs from Donor 2, 48 hours after infection of the cells with E1-deleted adenoviral vectors of the indicated adenoviral serotypes. B cells were identified as CD20+ lymphoid cells. Percent of cells that are GFP positive is shown. Cells were infected at an MOI of 500, 2000, and 5000 viral particles per cell. At each MOI, data is shown for Ad5, Ad7, Ad1, Ad34, Ad35, and Ad35++ (from left to right). Data represents infection efficiency.

### DETAILED DESCRIPTION

The present disclosure includes compositions and methods for selective targeting of hematopoietic cells (e.g., one or more particular types of hematopoietic cells). In particular, the present disclosure includes viral vectors that selective target one or more types of hematopoietic cells. In various embodiments, a viral vector that selectively targets one or more types of hematopoietic cells is an adenoviral vector of the present disclosure, e.g., an Ad3, 5, 7, 11, 14, 16, 21, 34, 35, 37, or 50 vector as disclosed herein. Various types of hematopoietic stem cells that can be targeted by vectors of the present disclosure are disclosed herein, including hematopoietic stem cell types, hematopoietic progenitor cell types, and further differentiated hematopoietic cell types including without limitation terminally differentiated hematopoietic cell types.

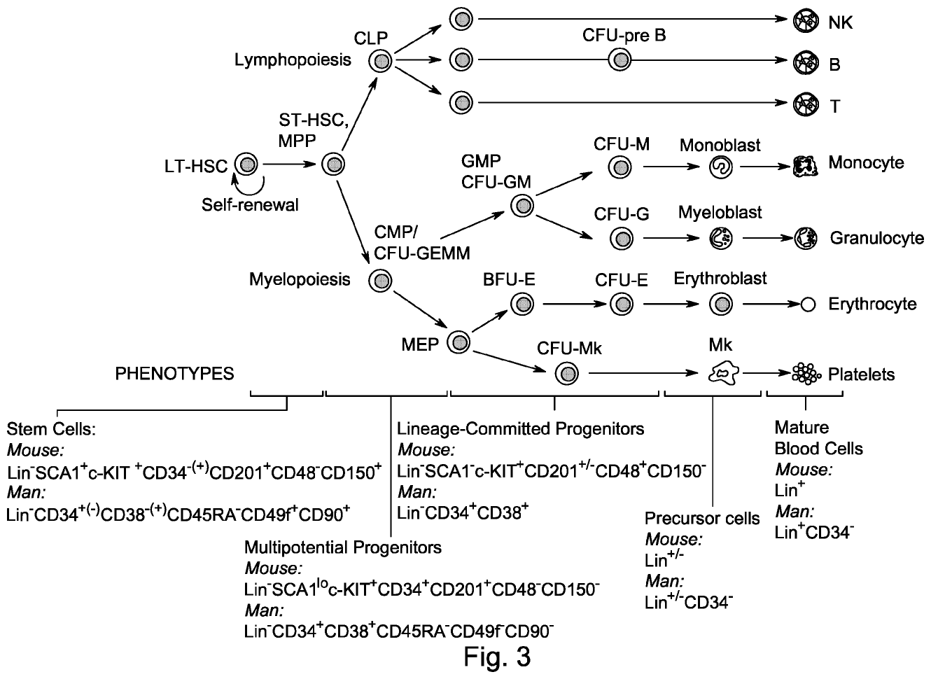

Hematopoiesis refers to the process by which various types of blood cells are produced. Because diverse cell types derive from hematopoietic stem and progenitor cells (HSPCs) through a process of differentiation, hematopoiesis is sometimes presented as a hierarchy. Hematopoietic stem cells (HSCs) are positioned at the “top” of this hierarchy (see FIG. 3). Without wishing to be bound by any particular scientific theory, HSCs are understood to be self-renewing and multipotent, differentiating into progenitors that further differentiate to produce mature and/or terminally differentiated blood cells. Stages of differentiation are disclosed herein (including, e.g., in FIG. 3), where further differentiation refers to increasing differentiation relative to an HSC or other temporally prior state and/or further change away from an HSC state as set forth in a differentiation lineage set forth in FIG. 3 or otherwise disclosed herein. According to some estimates, an adult human can include tens of thousands of HSCs, giving rise to hundreds of millions of progenitor cells that differentiate into precursor cells and eventually mature effector cells. Thus, a population of multipotent self-renewing HSCs generates large numbers of differentiated progeny by amplification and progressive lineage restriction. As referred to herein, hematopoietic cell types refer to any and all types of cells that are or derive from hematopoietic stem cells and/or hematopoietic progenitor cells, including without limitation particular cell types disclosed herein.

Without wishing to be bound by any particular scientific theory, HSCs can be divided into two subpopulations according to their CD34 expression: CD34⁺ long-term (LT)-HSCs and CD34⁺ short-term (ST)-HSCs. L-HSCs differentiate into ST-HSCs, and subsequently, ST-HSCs differentiate into multipotent progenitors (MPPs). In various embodiments, a hematopoietic cell type is or includes CD34⁺ hematopoietic cells.

Without wishing to be bound by any particular scientific theory, progenitors are understood to lack the capacity for self-renewal and are characterized by restricted differentiation, in that they can only yield cells of a particular lineage. Progenitors can be myeloid lineage progenitors or lymphoid lineage progenitors (referred to respectively as common myeloid progenitors (CMPs) and common lymphoid progenitors (CLPs)).

CMPs can differentiate into granulocyte-macrophage progenitors (GMPs) and megakaryocyte-erythrocyte progenitors (MEPs). GMPs can differentiate into granulocytes (e.g., neutrophils, eosinophils, and basophils), and monocytes (which can differentiate into to macrophages). MEPs can differentiate into megakaryocytes/platelets and erythrocytes. CLPs can differentiate into T, NK, and B cells.

Hematopoiesis further includes cell types that are referred to by names that are based on their identification in colony forming unit assays. Cells that form hematopoietic colonies (so-called CFUs or CFCs) can represent steps or stages of hematopoietic differentiation between HSCs and more terminally differentiated cells. CFUs can be identified by culturing hematopoietic cells in a semisolid media (typically methylcellulose or agar) supplemented with cytokines that promote the localized expansion and differentiation of hematopoietic cells in discrete colonies. CFUs can be identified by factors including, without limitation, the number of cells in a colony, the time required to produce the colony, and/or the types of cells in the colony. In general, without wishing to be bound by any particular scientific theory, progenitor cells can produce colonies that include, e.g., at least 30,000 cells including cell types of multiple lineages, e.g., by day 15-18 of culture. In various embodiments, culturing can produce colonies that generate erythroid bursts (e.g., of 5,000 cells), referred to as burst-forming unit erythroid (BFU-E). Other colony types can include granulomonocytic colonies (colony forming unit, granulomonocytic (CFU-GM)) and colonies of, e.g., 50-200 cells that are erythroid cells (colony-forming unit, erythroid (CFU-E)), granulocytic cells (CFU-G), or monocytic cells (CFU-M). These descriptions of colonies are solely for general illustration, and methods and techniques for colony analysis and identification are known in the art.

CLPs can also be referred to as CFU-L cells. In various embodiments, CFU-L cells can differentiate into CFU-B cells that differentiate into Pre-B Lymphocytes that can differentiate into B Lymphoblasts and subsequently into B Lymphocytes. In various embodiments, CFU-L cells can differentiate into CFU-T cells that differentiate into Pre-T Lymphocytes that can differentiate into T Lymphoblasts and subsequently into T Lymphocytes.

CMPs can also be referred to as CFU-GEMM cells. GMPs can also be referred to as CFU-GM cells. In various embodiments, CFU-GM cells can differentiate into CFU-M cells that differentiate into monoblasts and CFU-G cells that differentiate into neutrophils (e.g., via myeloblasts and neutrophilic myelocytes). MEPs can differentiate into BFU-E cells that can differentiate into CFU-E cells that can differentiate into erythroblasts (e.g., via rubriblasts, rubricytes, and metarubricytes). MEPs can differentiate into CFU-Mk cells that differentiate into megakaryocytes. CFU-Gemm can also differentiate into CFU-Eo cells that differentiate into eosinophils (e.g., via myeloblasts and eosinophilic myelocytes) and CFU-Baso cells that differentiate into basophils (e.g., via myeloblasts and basophilic myelocytes). Megakaryocyte lineage progenitors can include BFU-MK cells that differentiate into more mature progenitor cells referred to as CFU-MK cells.

HSC self-renewal and hematopoietic differentiation are controlled by multiple positive and negative regulatory elements, the mechanisms of which are poorly understood. Both intrinsic and extrinsic factors are likely involved, including for example epigenetic and microenvironmental factors, as well as intrinsic transcription factors (TFs) and extrinsic cytokines that contribute to stepwise differentiation of HSCs to mature blood cells.

Various means of identifying hematopoietic cell types are known in the art.

The present disclosure includes the recognition that gene therapy selectively targeting hematopoietic stem cells can be useful for long-term, transmissible modification of hematopoietic cells. The present disclosure includes the recognition that gene therapy selectively targeting hematopoietic progenitor cells can be useful for long-term, transmissible modification of hematopoietic cells. The present disclosure includes the recognition that gene therapy selectively targeting hematopoietic cells that are not stem cells and/or are not progenitor cells (e.g., terminally differentiated cells) can be useful for rapid therapeutic impact on one or more target cell types. For example, in various embodiments, differentiated cells can have more immediate effect because they do not require time to differentiate into effector cells. The present disclosure includes the recognition that gene therapy selectively targeting hematopoietic cells that are not stem cells and/or are not progenitor cells (e.g., terminally differentiated cells) can be useful for transient modification of a target cell type population. For example, in various embodiments, differentiated cells do not produce or constitute a long-term reservoir. The present disclosure includes the recognition that gene therapy selectively targeting hematopoietic cells that are not stem cells and/or are not progenitor cells (e.g., terminally differentiated cells) can be useful for target cell type-specific modification. For example, in various embodiments, differentiated cells do not produce cells of multiple lineages. The present disclosure includes the recognition that gene therapy selectively targeting hematopoietic cells that are not stem cells and/or are not progenitor cells (e.g., terminally differentiated cells) can be useful to minimize the targeting of a plurality of different cell types and thereby minimize risk of complications such as genotoxicity.

The present disclosure provides methods and compositions that include adenoviral vectors advantageous for gene therapy targeting hematopoietic cells (e.g., one or more particular types of hematopoietic cells). Methods and compositions of the present disclosure are based at least in part on the observation that adenoviral vectors of Ad3, Ad5, Ad7, Ad11, Ad14, Ad16, Ad21, Ad34, Ad35, Ad37, and Ad50 serotypes demonstrate certain advantageous properties for gene therapy targeting hematopoietic cells (e.g., one or more particular types of hematopoietic cells), at least as compared to one or more reference adenoviral vectors (e.g., an Ad5 vector or an Ad5/35 vector). Adenovirus (or, interchangeably, “adenoviral”) vectors include virus particles characterized by one or more adenoviral protein sequences and optionally include an adenoviral genome. Adenoviral genomes include nucleic acid sequences that include adenoviral sequences sufficient to (a) support packaging of the nucleic acid sequence (including conditional packaging) into an adenoviral vector and to (b) express a coding sequence. Adenoviral genomes can be linear, double-stranded DNA sequences and/or molecules. As those of skill in the art will appreciate, a linear genome such as an adenoviral genome can be present in a circular plasmid, e.g., for viral production purposes. Natural adenoviral genomes range from 26 kb to 45 kb in length, depending on the serotype.

The present disclosure includes methods and compositions that include engineered adenoviral vectors and adenoviral genomes. Adenoviral vectors include engineered adenoviral vectors that include an engineered adenoviral protein or engineered adenoviral genome. Engineered adenoviral genomes can be engineered to add or remove adenoviral genome sequences, e.g., as compared to a reference sequence.

In various embodiments, adenoviral serotypes and/or vectors of the present disclosure demonstrate increased infection of one or more hematopoietic cell type(s) as compared to infection of the hematopoietic cell type(s) by one or more reference adenoviral serotypes and/or vectors (e.g., Ad5 and/or Ad5/35), and are therefore useful, e.g., for targeting the hematopoietic cell type(s) for therapeutic purposes. In various embodiments, adenoviral serotypes and/or vectors of the present disclosure demonstrate increased infection of one or more hematopoietic cell type(s) as compared to infection of one or more reference hematopoietic cell type(s) by the same serotype and/or vector, and are therefore useful, e.g., for targeting the hematopoietic cell type(s) for therapeutic purposes. Methods and compositions of the present disclosure included adenoviral vectors of serotypes Ad3, Ad5, Ad7, Ad11, Ad14, Ad16, Ad21, Ad34, Ad35, Ad37, and Ad50.

### I. Gene Therapy Vectors

### 1(A). Adenoviral Vectors

The present disclosure includes adenoviral vectors and adenoviral genomes useful in gene therapy. Adenoviruses are large, icosahedral-shaped, non-enveloped viruses. Natural adenoviral capsids include three types of proteins: fiber, penton, and hexon. The hexon makes up the majority of the viral capsid, forming 20 triangular faces. A penton base is located at each of the 12 vertices of the capsid, and a fiber (also referred to as knobbed fiber) protrudes from each penton base. Penton and fiber, and in particular the fiber knob, are of particular importance in receptor binding and internalization as they facilitate the attachment of the capsid to host cells.

Adenoviral genomes include Adenoviral DNA flanked on both ends by serotype-specific inverted terminal repeats (ITRs), which are understood to be cis elements that contribute to or are necessary for viral genome replication and packaging. Depending on the serotype, ITRs can be approximately 100-200 base pairs (e.g., about 160 base pairs) in length, with highest conservation at nucleotide positions (e.g., ˜50 base pairs) closest to the adenoviral genome terminii. Adenoviral genomes also include a packaging sequence (e.g., a conditional or non-conditional packaging sequence), which can facilitate packaging of the viral genome into viral vectors. Packaging sequences are located in the left portion of the genome.

Natural adenoviral genomes encode several proteins including early transcriptional units, E1, E2, E3, and E4 and late transcriptional units which encode structural protein components of the adenoviral vector. Early (E) and late (L) transcription are divided by the onset of viral genome replication. The E1 region (E1A and E1B) encodes proteins responsible for the regulation of transcription of the viral genome. The expression of the E2 region (E2A and E2B) results in the synthesis of the proteins for viral genome replication. These proteins are involved in DNA replication, late gene expression, and host cell shut-off. The products of the late genes, including the majority of the viral capsid proteins, are expressed only after significant processing of a single primary transcript issued by the major late promoter (MLP). The MLP is particularly efficient during the late phase of infection. mRNAs transcribed using this promoter can include a 5′-tripartite leader (TPL) sequence that facilitates translation.

### 1(B). Ad3, 5, 7, 11, 14, 16, 21, 34, 35, 37, and 50 Gene Therapy Vectors

The present disclosure includes Ad3, 5, 7, 11, 14, 16, 21, 34, 35, 37, or 50 genomes. In various embodiments, an Ad3, 5, 7, 11, 14, 16, 21, 34, 35, 37, or 50 genome is a single-stranded or double-stranded DNA sequence that includes ITRs of an Ad3, 5, 7, 11, 14, 16, 21, 34, 35, 37, or 50 vector (e.g., a 5′ ITR according to SEQ ID NO: 1, 19, 37, 55, 73, 91, 109, 127, 145, 163, or 181 and a 3′ ITR according to SEQ ID NO: 2, 20, 38, 56, 74, 92, 110, 128, 146, 164, or 182), or ITRs that individually and/or together have at least 75% sequence identity (e.g., at least 75%, 80%, 85%, 90%, 95%, 96%, 97%, 98%, 99%, or 100% identity) thereto. In various embodiments, an Ad3, 5, 7, 11, 14, 16, 21, 34, 35, 37, or 50 genome is a single-stranded or double-stranded DNA sequence that includes a packaging sequence of an Ad3, 5, 7, 11, 14, 16, 21, 34, 35, 37, or 50 vector (e.g., a packaging sequence according to SEQ ID NO: 3, 21, 39, 57, 75, 93, 111, 129, 147, 165, or 183), or a packaging sequence having at least 75% sequence identity (e.g., at least 75%, 80%, 85%, 90%, 95%, 96%, 97%, 98%, 99%, or 100% identity) to the entirety or a portion thereof. In various embodiments, an Ad3, 5, 7, 11, 14, 16, 21, 34, 35, 37, or 50 genome is a single-stranded or double-stranded DNA sequence that includes a sequence with at least 75% sequence identity (e.g., at least 75%, 80%, 85%, 90%, 95%, 96%, 97%, 98%, 99%, or 100% identity) to all, a portion of, or a contiguous corresponding portion of, or a discontiguous corresponding portion of a reference Ad3, 5, 7, 11, 14, 16, 21, 34, 35, 37, or 50 genome (e.g., SEQ ID NO: 199, 200, 201, 202, 203, 204, 205, 206, 207, 208, or 209).

In various embodiments, an Ad3, 5, 7, 11, 14, 16, 21, 34, 35, 37, or 50 genome is any nucleotide sequence that includes at least ITRs of an Ad3, 5, 7, 11, 14, 16, 21, 34, 35, 37, or 50 vector (e.g., a 5′ ITR according to SEQ ID NO: 1, 19, 37, 55, 73, 91, 109, 127, 145, 163, or 181 and a 3′ ITR according to SEQ ID NO: 2, 20, 38, 56, 74, 92, 110, 128, 146, 164, or 182), or ITRs that individually and/or together have at least 75% sequence identity (e.g., at least 75%, 80%, 85%, 90%, 95%, 96%, 97%, 98%, 99%, or 100% identity) thereto. In various embodiments, an Ad3, 5, 7, 11, 14, 16, 21, 34, 35, 37, or 50 genome is an Ad3, 5, 7, 11, 14, 16, 21, 34, 35, 37, or 50 genome from which one or more nucleotides, coding sequences, and/or genes are completely or partially deleted as compared to a reference sequence. For example, in some embodiments, an Ad3, 5, 7, 11, 14, 16, 21, 34, 35, 37, or 50 genome can be a genome that does not include one or more of E1, E2, E3, and E4. In certain embodiments, an Ad3, 5, 7, 11, 14, 16, 21, 34, 35, 37, or 50 genome is a genome that does not include any coding sequences of an Ad3, 5, 7, 11, 14, 16, 21, 34, 35, 37, or 50 genome (e.g., a “gutless” vector that includes ITRs having at least 75% sequence identity to Ad3, 5, 7, 11, 14, 16, 21, 34, 35, 37, or 50 genome ITRs but includes none of the coding sequences present in a reference Ad3, 5, 7, 11, 14, 16, 21, 34, 35, 37, or 50 genome).

In various embodiments, an Ad3, 5, 7, 11, 14, 16, 21, 34, 35, 37, or 50 genome includes, does not include, or includes a deletion of, all or a portion of an E1 sequence according to SEQ ID NO: 4, 22, 40, 58, 76, 94, 112, 130, 148, 166, or 184, or a sequence having at least 75% sequence identity (e.g., at least 75%, 80%, 85%, 90%, 95%, 96%, 97%, 98%, 99%, or 100% identity) thereto.

In various embodiments, an Ad3, 5, 7, 11, 14, 16, 21, 34, 35, 37, or 50 genome includes, does not include, or includes a deletion of, all or a portion of an E2 sequence according to SEQ ID NO: 5, 23, 41, 59, 77, 95, 113, 131, 149, 167, or 185, or a sequence having at least 75% sequence identity (e.g., at least 75%, 80%, 85%, 90%, 95%, 96%, 97%, 98%, 99%, or 100% identity) thereto.

In various embodiments, an Ad3, 5, 7, 11, 14, 16, 21, 34, 35, 37, or 50 genome includes, does not include, or includes a deletion of, all or a portion of an E3 sequence according to SEQ ID NO: 6, 24, 42, 60, 78, 96, 114, 132, 150, 168, or 186, or a sequence having at least 75% sequence identity (e.g., at least 75%, 80%, 85%, 90%, 95%, 96%, 97%, 98%, 99%, or 100% identity) thereto.

In various embodiments, an Ad3, 5, 7, 11, 14, 16, 21, 34, 35, 37, or 50 genome includes, or does not include, a sequence that encodes a fiber, where the sequence has at least 75% sequence identity (e.g., at least 75%, 80%, 85%, 90%, 95%, 96%, 97%, 98%, 99%, or 100% identity) to SEQ ID NO: 7, 25, 43, 61, 79, 97, 115, 133, 151, 169, or 187.

In various embodiments, an Ad3, 5, 7, 11, 14, 16, 21, 34, 35, 37, or 50 genome includes, or does not include, a sequence that encodes a fiber shaft, where the sequence has at least 75% sequence identity (e.g., at least 75%, 80%, 85%, 90%, 95%, 96%, 97%, 98%, 99%, or 100% identity) to SEQ ID NO: 9, 27, 45, 63, 81, 99, 117, 135, 153, 171, or 189.

In various embodiments, an Ad3, 5, 7, 11, 14, 16, 21, 34, 35, 37, or 50 genome includes, or does not include, a sequence that encodes a fiber knob, where the sequence has at least 75% sequence identity (e.g., at least 75%, 80%, 85%, 90%, 95%, 96%, 97%, 98%, 99%, or 100% identity) to SEQ ID NO: 10, 28, 46, 64, 82, 100, 118, 136, 154, 172, or 190.

In various embodiments, an Ad3, 5, 7, 11, 14, 16, 21, 34, 35, 37, or 50 genome includes, or does not include, a sequence that encodes a fiber tail, where the sequence has at least 75% sequence identity (e.g., at least 75%, 80%, 85%, 90%, 95%, 96%, 97%, 98%, 99%, or 100% identity) to SEQ ID NO: 8, 26, 44, 62, 80, 98, 116, 134, 152, 170, or 188.

In various embodiments, an Ad3, 5, 7, 11, 14, 16, 21, 34, 35, 37, or 50 genome includes, or does not include, a sequence that encodes a penton, where the sequence has at least 75% sequence identity (e.g., at least 75%, 80%, 85%, 90%, 95%, 96%, 97%, 98%, 99%, or 100% identity) to SEQ ID NO: 11, 29, 47, 65, 83, 101, 119, 137, 155, 173, or 191.

In various embodiments, an Ad3, 5, 7, 11, 14, 16, 21, 34, 35, 37, or 50 genome includes, or does not include, a sequence that encodes a hexon, where the sequence has at least 75% sequence identity (e.g., at least 75%, 80%, 85%, 90%, 95%, 96%, 97%, 98%, 99%, or 100% identity) to SEQ ID NO: 12, 30, 48, 66, 84, 102, 120, 138, 156, 174, or 192.

The present disclosure includes Ad3, 5, 7, 11, 14, 16, 21, 34, 35, 37, or 50 vectors that include a fiber having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber (e.g., a fiber according to SEQ ID NO: 13, 31, 49, 67, 85, 103, 121, 139, 157, 175, or 193).

The present disclosure includes Ad3, 5, 7, 11, 14, 16, 21, 34, 35, 37, or 50 vectors that include a fiber tail having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber tail (e.g., a fiber tail according to SEQ ID NO: 18, 36, 54, 72, 90, 108, 126, 144, 162, 180, or 198, e.g., where the fiber tail is the portion of the fiber including all amino acids N-terminal to the fiber shaft).

The present disclosure includes Ad3, 5, 7, 11, 14, 16, 21, 34, 35, 37, or 50 vectors that include a fiber shaft having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber shaft (e.g., a fiber shaft according to SEQ ID NO: 14, 32, 50, 68, 86, 104, 122, 140, 158, 176, or 194).

The present disclosure includes Ad3, 5, 7, 11, 14, 16, 21, 34, 35, 37, or 50 vectors that include a fiber knob having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber knob (e.g., a fiber knob according to SEQ ID NO: 15, 33, 51, 69, 87, 105, 123, 141, 159, 177, or 195).

The present disclosure includes Ad3, 5, 7, 11, 14, 16, 21, 34, 35, 37, or 50 vectors that include a penton having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 penton (e.g., a penton according to SEQ ID NO: 16, 34, 52, 70, 88, 106, 124, 142, 160, 178, or 196).

The present disclosure includes Ad3, 5, 7, 11, 14, 16, 21, 34, 35, 37, or 50 vectors that include a hexon having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 hexon (e.g., a hexon according to SEQ ID NO: 17, 35, 53, 71, 89, 107, 125, 143, 161, 179, or 197).

In various embodiments, an Ad3, 5, 7, 11, 14, 16, 21, 34, 35, 37, or 50 vector is any adenoviral vector that includes at least a fiber having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber (e.g., a fiber according to SEQ ID NO: 13, 31, 49, 67, 85, 103, 121, 139, 157, 175, or 193).

In various embodiments, an Ad3, 5, 7, 11, 14, 16, 21, 34, 35, 37, or 50 vector is any adenoviral vector that includes at least a fiber tail having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber tail (e.g., a fiber tail according to SEQ ID NO: 18, 36, 54, 72, 90, 108, 126, 144, 162, 180, or 198, e.g., where the fiber tail is the portion of the fiber including all amino acids N-terminal to the fiber shaft).

In various embodiments, an Ad3, 5, 7, 11, 14, 16, 21, 34, 35, 37, or 50 vector is any adenoviral vector that includes at least a fiber shaft having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber shaft (e.g., a fiber shaft according to SEQ ID NO: 14, 32, 50, 68, 86, 104, 122, 140, 158, 176, or 194).

In various embodiments, an Ad3, 5, 7, 11, 14, 16, 21, 34, 35, 37, or 50 vector is any adenoviral vector that includes at least a fiber knob having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber knob (e.g., a fiber knob according to SEQ ID NO: 15, 33, 51, 69, 87, 105, 123, 141, 159, 177, or 195).

In various embodiments, an Ad3, 5, 7, 11, 14, 16, 21, 34, 35, 37, or 50 vector is any adenoviral vector that includes at least a penton having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 penton (e.g., a penton according to SEQ ID NO: 16, 34, 52, 70, 88, 106, 124, 142, 160, 178, or 196).

In various embodiments, an Ad3, 5, 7, 11, 14, 16, 21, 34, 35, 37, or 50 vector is any adenoviral vector that includes at least a hexon having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 hexon (e.g., a hexon according to SEQ ID NO: 17, 35, 53, 71, 89, 107, 125, 143, 161, 179, or 197).

Thus, an Ad3, 5, 7, 11, 14, 16, 21, 34, 35, 37, or 50 vector can be a chimeric adenoviral vector that includes at least a fiber knob having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber knob and at least one protein or portion thereof (such as a fiber shaft, fiber tail, penton, or hexon) that has at least 75% sequence identity (e.g., at least 75%, 80%, 85%, 90%, 95%, 96%, 97%, 98%, 99%, or 100% identity) to a different adenoviral serotype.

An Ad3, 5, 7, 11, 14, 16, 21, 34, 35, 37, or 50 vector can be a chimeric adenoviral vector that includes at least a fiber shaft having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber shaft and at least one protein or portion thereof (such as a fiber knob, fiber tail, penton, or hexon) that has at least 75% sequence identity (e.g., at least 75%, 80%, 85%, 90%, 95%, 96%, 97%, 98%, 99%, or 100% identity) to a different adenoviral serotype.

An Ad3, 5, 7, 11, 14, 16, 21, 34, 35, 37, or 50 vector can be a chimeric adenoviral vector that includes at least a fiber tail having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber tail and at least one protein or portion thereof (such as a fiber knob, fiber shaft, penton, or hexon) that has at least 75% sequence identity (e.g., at least 75%, 80%, 85%, 90%, 95%, 96%, 97%, 98%, 99%, or 100% identity) to a different adenoviral serotype.

An Ad3, 5, 7, 11, 14, 16, 21, 34, 35, 37, or 50 vector can be a chimeric adenoviral vector that includes at least a penton having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 penton and at least one protein or portion thereof (such as a fiber knob, fiber shaft, fiber tail, or hexon) that has at least 75% sequence identity (e.g., at least 75%, 80%, 85%, 90%, 95%, 96%, 97%, 98%, 99%, or 100% identity) to a different adenoviral serotype.

An Ad3, 5, 7, 11, 14, 16, 21, 34, 35, 37, or 50 vector can be a chimeric adenoviral vector that includes at least a hexon having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 hexon and at least one protein or portion thereof (such as a fiber knob, fiber shaft, fiber tail, or penton) that has at least 75% sequence identity (e.g., at least 75%, 80%, 85%, 90%, 95%, 96%, 97%, 98%, 99%, or 100% identity) to a different adenoviral serotype.

Exemplary sequences of Ad3, 5, 7, 11, 14, 16, 21, 34, 35, 37, or 50 components (e.g., ITRs, packaging sequences, genes, and proteins) are provided in the following tables. Viral polypeptides include proteins that are components of viral vectors and portions or fragments thereof, including for example a fiber, fiber knob, fiber shaft, fiber tail, penton, or hexon.

In various embodiments, an Ad35 fiber knob of an Ad35 vector or chimeric Ad vector that includes an Ad35 fiber knob is a mutant Ad35 fiber knob. In particular embodiments, a mutant Ad35 fiber knob is an Ad35++ mutant fiber knob (alternatively referred to herein as an Ad35++ fiber knob). In various embodiments, an Ad35++ mutant fiber knob is an Ad35 fiber knob mutated to increase the affinity to CD46, e.g., by 25-fold, e.g., such that the Ad35++ mutant fiber knob increases cell transduction efficiency, e.g., at lower multiplicity of infection (MOI) (Li and Lieber, FEBS Letters, 593(24): 3623-3648, 2019). In various embodiments, an Ad35++ mutant fiber knob includes at least one mutation selected from Ile192Val, Asp207Gly (or Glu207Gly in certain Ad35 sequences), Asn217Asp, Thr226Ala, Thr245Ala, Thr254Pro, Ile256Leu, Ile256Val, Arg259Cys, and Arg279His. In various embodiments, an Ad35++ mutant fiber knob includes each of the following mutations: Ile192Val, Asp207Gly (or Glu207Gly in certain Ad35 sequences), Asn217Asp, Thr226Ala, Thr245Ala, Thr254Pro, Ile256Leu, Ile256Val, Arg259Cys, and Arg279His. In various embodiments, amino acid numbering of an Ad35 fiber is according to GenBank accession no. AP_000601 or an amino acid sequence corresponding thereto, e.g., where position 207 is Glu or Asp. In various embodiments, an Ad35 fiber has an amino acid sequence according to GenBank accession no. AP_000601. Further description of Ad35++ fiber knob mutations is found in Wang 2008 J. Virol. 82(21): 10567-10579, which is incorporated herein by reference in its entirety and with respect to fiber knobs. The present disclosure includes, for example, a recombinant Ad35 vector with a mutant Ad35 fiber knob or an Ad5/35 vector with a mutant Ad35 fiber knob.

In various embodiments, an adenoviral vector or genome of the present disclosure can be an adenoviral vector and/or genome disclosed in WO 2021/003432, which is herein incorporated by reference in its entirety, and particularly with respect to adenoviral vectors and genomes.

Various sequences corresponding to accession numbers disclosed herein, including e.g., accession numbers referred to herein as SEQ ID NOs: 199, 200, 201, 202, 203, 204, 205, 206, 207, 208, and/or 209 as indicated in Tables 1-22, are provided herein in the below listing of accession sequences. Those of skill in the art will appreciate that such sequences, including the sequences disclosed in the below listing of accession sequences, can be referenced in whole (e.g., by an accession number) or in part (e.g., by reference to a nucleotide position and/or a set or range of nucleotide positions of a sequence and/or accession number).

TABLE 1
Ad3 Genomic Sequences
Ad3 Genomic Sequences
Reference Ad3 Genome Sequence: GenBank
accession no. NC_011203 (SEQ ID NO: 199)

| | Exemplary Sequence | |
|---|---|---|
| Component | (position in reference) | SEQ ID NO: |
| Ad3 5′ (left) ITR | 1-136 | 1 |
| Ad3 3′ (right) ITR | 35208-35343 | 2 |
| Ad3 Packaging | 137-479 | 3 |
| Sequence | | |
| Ad3 E1 | 480-3918 | 4 |
| Ad3 E2 | 26643-3947 | 5 |
| Ad3 E3 | 27085-31186 | 6 |
| Ad3 fiber | 31368-32327 | 7 |
| Ad3 fiber tail | 31368-31493 | 8 |
| Ad3 fiber shaft | 31494-31763 | 9 |
| Ad3 fiber knob | 31764-32324 | 10 |
| Ad3 penton | 13905-15539 | 11 |
| Ad3 hexon | 18418-21252 | 12 |

TABLE 2
Ad3 Amino Acid Sequences
Ad3 Amino Acid Sequences

| | | SEQ |
|---|---|---|
| | Exemplary Sequence | ID |
| Component | (position in reference) | NO: |
| Ad3 fiber | 1-319 (GenBank accession no. YP_002213796) | 13 |
| Ad3 fiber shaft | 43-132 (GenBank accession no. YP_002213796) | 14 |
| Ad3 fiber knob | 134-319 (GenBank accession no. | 15 |
| | YP_002213796) | |
| Ad3 penton | 1-544 (GenBank accession no. YP_002213774) | 16 |
| Ad3 hexon | 1-944 (GenBank accession no. YP_002213779) | 17 |
| Ad3 fiber tail | 1-42 (GenBank accession no. YP_002213796) | 18 |

TABLE 3
Ad7 Genomic Sequences
Ad7 Genomic Sequences
Reference Ad7 Genome Sequence: GenBank
accession no. AC_000018 (SEQ ID NO: 200)

| | Exemplary Sequence | |
|---|---|---|
| Component | (position in reference) | SEQ ID NO: |
| Ad7 5′ (left) ITR | 1-136 | 19 |
| Ad7 3′ (right) ITR | 35379-35514 | 20 |
| Ad7 Packaging | 137-479 | 21 |
| Sequence | | |
| Ad7 E1 | 480-3919 | 22 |
| Ad7 E2 | 26867-3947 | 23 |
| Ad7 E3 | 27308-31345 | 24 |
| Ad7 fiber | 31529-32506 | 25 |
| Ad7 fiber tail | 31529-31654 | 26 |
| Ad7 fiber shaft | 31655-31927 | 27 |
| Ad7 fiber knob | 31928-32503 | 28 |
| Ad7 penton | 14153-15787 | 29 |
| Ad7 hexon | 18666-21470 | 30 |

TABLE 4
Ad7 Amino Acid Sequences
Ad7 Amino Acid Sequences

| | Exemplary Sequence | SEQ |
|---|---|---|
| Component | (position in reference) | ID NO: |
| Ad7 fiber | 1-325 (GenBank accession no. AP_000564) | 31 |
| Ad7 fiber shaft | 43-133 (GenBank accession no. AP_000564) | 32 |
| Ad7 fiber knob | 134-325 (GenBank accession no. AP_000564) | 33 |
| Ad7 penton | 1-544 (GenBank accession no. AP_000543) | 34 |
| Ad7 hexon | 1-934 (GenBank accession no. AP_000548) | 35 |
| Ad7 fiber tail | 1-42 (GenBank accession no. AP_000564) | 36 |

TABLE 5
Ad11 Genomic Sequences
Ad11 Genomic Sequences
Reference Ad11 Genome Sequence: GenBank
accession no. NC_011202 (SEQ ID NO: 201)

| | Exemplary Sequence | |
|---|---|---|
| Component | (position in reference) | SEQ ID NO: |
| Ad11 5′ (left) ITR | 1-137 | 37 |
| Ad11 3′ (right) ITR | 34658-34794 | 38 |
| Ad11 Packaging | 138-479 | 39 |
| Sequence | | |
| Ad11 E1 | 480-3931 | 40 |
| Ad11 E2 | 25445-3963 | 41 |
| Ad11 E3 | 26866-30624 | 42 |
| Ad11 fiber | 30811-31788 | 43 |
| Ad11 fiber tail | 30811-30936 | 44 |
| Ad11 fiber shaft | 30937-31209 | 45 |
| Ad11 fiber knob | 31210-31785 | 46 |
| Ad11 penton | 13682-15367 | 47 |
| Ad11 hexon | 18254-21100 | 48 |

TABLE 6
Ad11 Amino Acid Sequences
Ad11 Amino Acid Sequences

| | Exemplary Sequence | |
|---|---|---|
| Component | (position in reference) | SEQ ID NO: |
| Ad11 fiber | 1-325 (GenBank accession no. YP_002213828) | 49 |
| Ad11 fiber shaft | 43-133 (GenBank accession no. YP_002213828) | 50 |
| Ad11 fiber knob | 134-325 (GenBank accession no. YP_002213828) | 51 |
| Ad11 penton | 1-561 (GenBank accession no. YP_002213807) | 52 |
| Ad11 hexon | 1-948 (GenBank accession no. YP_002213812) | 53 |
| Ad11 fiber tail | 1-42 (GenBank accession no. YP_002213828) | 54 |

TABLE 7
Ad14 Genomic Sequences
Ad14 Genomic Sequences
Reference Ad14 Genome Sequence: GenBank
accession no. AY803294 (SEQ ID NO: 202)

| | Exemplary Sequence | |
|---|---|---|
| Component | (position in reference) | SEQ ID NO: |
| Ad14 5′ (left) ITR | 1-137 | 55 |
| Ad14 3′ (right) ITR | 34628-34764 | 56 |
| Ad14 Packaging | 138-479 | 57 |
| Sequence | | |
| Ad14 E1 | 480-3947 | 58 |
| Ad14 E2 | 23389-3963 | 59 |
| Ad14 E3 | 26854-30601 | 60 |
| Ad14 fiber | 30788-31765 | 61 |
| Ad14 fiber tail | 30788-30913 | 62 |
| Ad14 fiber shaft | 30914-31186 | 63 |
| Ad14 fiber knob | 31187-31762 | 64 |
| Ad14 penton | 13698-15374 | 65 |
| Ad14 hexon | 18252-21089 | 66 |

TABLE 8
Ad14 Amino Acid Sequences
Ad14 Amino Acid Sequences

| | | SEQ |
|---|---|---|
| | Exemplary Sequence | ID |
| Component | (position in reference) | NO: |
| Ad14 fiber | 1-325 (GenBank accession no. AAW33140) | 67 |
| Ad14 fiber shaft | 43-133 (GenBank accession no. AAW33140) | 68 |
| Ad14 fiber knob | 134-325 (GenBank accession no. | 69 |
| | AAW33140) | |
| Ad14 penton | 1-558 (GenBank accession no. AAW33119) | 70 |
| Ad14 hexon | 1-945 (GenBank accession no. AAW33124) | 71 |
| Ad14 fiber tail | 1-42 (GenBank accession no. AAW33140) | 72 |

TABLE 9
Ad16 Genomic Sequences
Ad16 Genomic Sequences
Reference Ad16 Genome Sequence: GenBank
accession no. AY601636 (SEQ ID NO: 203)

| | Exemplary Sequence | |
|---|---|---|
| Component | (position in reference) | SEQ ID NO: |
| Ad16 5′ (left) ITR | 1-114 | 73 |
| Ad16 3′ (right) ITR | 35409-35522 | 74 |
| Ad16 Packaging | 115-479 | 75 |
| Sequence | | |
| Ad16 E1 | 480-3910 | 76 |
| Ad16 E2 | 23580-3954 | 77 |
| Ad16 E3 | 27107-31263 | 78 |
| Ad16 fiber | 31448-32509 | 79 |
| Ad16 fiber tail | 31448-31573 | 80 |
| Ad16 fiber shaft | 31574-31933 | 81 |
| Ad16 fiber knob | 31934-32506 | 82 |
| Ad16 penton | 13902-17534 | 83 |
| Ad16 hexon | 18450-21272 | 84 |

TABLE 10
Ad16 Amino Acid Sequences
Ad16 Amino Acid Sequences

| | | SEQ |
|---|---|---|
| | Exemplary Sequence | ID |
| Component | (position in reference) | NO: |
| Ad16 fiber | 1-353 (GenBank accession no. AAW33461) | 85 |
| Ad16 fiber shaft | 43-172 (GenBank accession no. AAW33461) | 86 |
| Ad16 fiber knob | 173-353 (GenBank accession no. | 87 |
| | AAW33461) | |
| Ad16 penton | 1-555 (GenBank accession no. AAW33439) | 88 |
| Ad16 hexon | 1-940 (GenBank accession no. AAW33444) | 89 |
| Ad16 fiber tail | 1-42 (GenBank accession no. AAW33461) | 90 |

TABLE 11
Ad21 Genomic Sequences
Ad21 Genomic Sequences
Reference Ad21 Genome Sequence: GenBank
accession no. AY601633 (SEQ ID NO: 204)

| | Exemplary Sequence | |
|---|---|---|
| Component | (position in reference) | SEQ ID NO: |
| Ad21 5′ (left) ITR | 1-114 | 91 |
| Ad21 3′ (right) ITR | 35269-35382 | 92 |
| Ad21 Packaging | 115-479 | 93 |
| Sequence | | |
| Ad21 E1 | 480-3911 | 94 |
| Ad21 E2 | 23611-3924 | 95 |
| Ad21 E3 | 27441-31208 | 96 |
| Ad21 fiber | 31406-32377 | 97 |
| Ad21 fiber tail | 31406-31531 | 98 |
| Ad21 fiber shaft | 31532-31804 | 99 |
| Ad21 fiber knob | 31805-32374 | 100 |
| Ad21 penton | 13878-15563 | 101 |
| Ad21 hexon | 18454-21303 | 102 |

TABLE 12
Ad21 Amino Acid Sequences
Ad21 Amino Acid Sequences

| | Exemplary Sequence | SEQ |
|---|---|---|
| Component | (position in reference) | ID NO: |
| Ad21 fiber | 1-323 (GenBank accession no. AAW33370) | 103 |
| Ad21 fiber | 43-133 (GenBank accession no. AAW33370) | 104 |
| shaft | | |
| Ad21 fiber | 134-323 (GenBank accession no. AAW33370) | 105 |
| knob | | |
| Ad21 penton | 1-561 (GenBank accession no. AAW33349) | 106 |
| Ad21 hexon | 1-949 (GenBank accession no. AAW33354) | 107 |
| Ad21 fiber | 1-42 (GenBank accession no. AAW33370) | 108 |
| tail | | |

TABLE 13
Ad34 Genomic Sequences
Ad34 Genomic Sequences
Reference Ad34 Genome Sequence: GenBank
accession no. AY737797 (SEQ ID NO: 205)

| | Exemplary Sequence | |
|---|---|---|
| Component | (position in reference) | SEQ ID NO: |
| Ad34 5′ (left) ITR | 1-137 | 109 |
| Ad34 3′ (right) ITR | 34639-34775 | 110 |
| Ad34 Packaging | 138-479 | 111 |
| Sequence | | |
| Ad34 E1 | 480-3929 | 112 |
| Ad34 E2 | 23399-3945 | 113 |
| Ad34 E3 | 27185-30625 | 114 |
| Ad34 fiber | 30812-31783 | 115 |
| Ad34 fiber tail | 30812-30937 | 116 |
| Ad34 fiber shaft | 30938-31210 | 117 |
| Ad34 fiber knob | 31211-31780 | 118 |
| Ad34 penton | 13681-15357 | 119 |
| Ad34 hexon | 18244-21099 | 120 |

TABLE 14
Ad34 Amino Acid Sequences
Ad34 Amino Acid Sequences

| | Exemplary Sequence | SEQ |
|---|---|---|
| Component | (position in reference) | ID NO: |
| Ad34 fiber | 1-323 (GenBank accession no. AAW33501) | 121 |
| Ad34 fiber | 43-133 (GenBank accession no. AAW33501) | 122 |
| shaft | | |
| Ad34 fiber | 134-323 (GenBank accession no. AAW33501) | 123 |
| knob | | |
| Ad34 penton | 1-558 (GenBank accession no. ABC49791) | 124 |
| Ad34 hexon | 1-951 (GenBank accession no. AAW33485) | 125 |
| Ad34 fiber | 1-42 (GenBank accession no. AAW33501) | 126 |
| tail | | |

TABLE 15
Ad37 Genomic Sequences
Ad37 Genomic Sequences
Reference Ad37 Genome Sequence: GenBank
accession no. DQ900900 (SEQ ID NO: 206)

| | Exemplary Sequence | |
|---|---|---|
| Component | (position in reference) | SEQ ID NO: |
| Ad37 5′ (left) ITR | 1-159 | 127 |
| Ad37 3′ (right) ITR | 35055-35213 | 128 |
| Ad37 Packaging | 160-479 | 129 |
| Sequence | | |
| Ad37 E1 | 480-3867 | 130 |
| Ad37 E2 | 22777-3902 | 131 |
| Ad37 E3 | 26198-30771 | 132 |
| Ad37 fiber | 31038-32135 | 133 |
| Ad37 fiber tail | 31038-31163 | 134 |
| Ad37 fiber shaft | 31164-31592 | 135 |
| Ad37 fiber knob | 31593-32132 | 136 |
| Ad37 penton | 13530-15089 | 137 |
| Ad37 hexon | 17775-20624 | 138 |

TABLE 16
Ad37 Amino Acid Sequences
Ad37 Amino Acid Sequences

| | Exemplary Sequence | SEQ |
|---|---|---|
| Component | (position in reference) | ID NO: |
| Ad37 fiber | 1-361 (GenBank accession no. ABK59080) | 139 |
| Ad37 fiber | 43-185 (GenBank accession no. ABK59080) | 140 |
| shaft | | |
| Ad37 fiber | 186-361 (GenBank accession no. ABK59080) | 141 |
| knob | | |
| Ad37 penton | 1-519 (GenBank accession no. ABK59086) | 142 |
| Ad37 hexon | 1-949 (GenBank accession no. ABK59070) | 143 |
| Ad37 fiber | 1-42 (GenBank accession no. ABK59080) | 144 |
| tail | | |

TABLE 17
Ad50 Genomic Sequences
Ad50 Genomic Sequences
Reference Ad50 Genome Sequence: GenBank
accession no. AY737798 (SEQ ID NO: 207)

| | Exemplary Sequence | |
|---|---|---|
| Component | (position in reference) | SEQ ID NO: |
| Ad50 5′ (left) ITR | 1-114 | 145 |
| Ad50 3′ (right) ITR | 35272-35385 | 146 |
| Ad50 Packaging | 115-479 | 147 |
| Sequence | | |
| Ad50 E1 | 480-3910 | 148 |
| Ad50 E2 | 23590-3923 | 149 |
| Ad50 E3 | 27102-31222 | 150 |
| Ad50 fiber | 31409-32380 | 151 |
| Ad50 fiber tail | 31409-31534 | 152 |
| Ad50 fiber shaft | 31535-31807 | 153 |
| Ad50 fiber knob | 31808-32377 | 154 |
| Ad50 penton | 13888-15570 | 155 |
| Ad50 hexon | 18460-21282 | 156 |

TABLE 18
Ad50 Amino Acid Sequences
Ad50 Amino Acid Sequences

| | Exemplary Sequence | SEQ |
|---|---|---|
| Component | (position in reference) | ID NO: |
| Ad50 fiber | 1-323 (GenBank accession no. AAW33547) | 157 |
| Ad50 fiber | 43-133 (GenBank accession no. AAW33547) | 158 |
| shaft | | |
| Ad50 fiber | 134-323 (GenBank accession no. AAW33547) | 159 |
| knob | | |
| Ad50 penton | 1-560 (GenBank accession no. AAW33525) | 160 |
| Ad50 hexon | 1-940 (GenBank accession no. AAW33530) | 161 |
| Ad50 fiber | 1-42 (GenBank accession no. AAW33547) | 162 |
| tail | | |

TABLE 19
Ad5 Genomic Sequences
Ad5 Genomic Sequences
Reference Ad5 Genome Sequence: GenBank
accession no. AC_000008 (SEQ ID NO: 208)

| | Exemplary Sequence | |
|---|---|---|
| Component | (position in reference) | SEQ ID NO: |
| Ad5 5′ (left) ITR | 1-103 | 163 |
| Ad5 3′ (right) ITR | 35836-35938 | 164 |
| Ad5 Packaging | 104-479 | 165 |
| Sequence | | |
| Ad5 E1 | 480-3509 | 166 |
| Ad5 E2 | 4091-24032 | 167 |
| Ad5 E3 | 27174-30839 | 168 |
| Ad5 fiber | 31042-32787 | 169 |
| Ad5 fiber tail | 31042-31170 | 170 |
| Ad5 fiber shaft | 31171-32241 | 171 |
| Ad5 fiber knob | 32242-32784 | 172 |
| Ad5 penton | 14156-15871 | 173 |
| Ad5 hexon | 18842-21700 | 174 |

TABLE 20
Ad5 Amino Acid Sequences
Ad5 Amino Acid Sequences

| | Exemplary Sequence | SEQ |
|---|---|---|
| Component | (position in reference) | ID NO: |
| Ad5 fiber | 1-581 (GenBank accession no. AP_000226) | 175 |
| Ad5 fiber | 44-400 (GenBank accession no. AP_000226) | 176 |
| shaft | | |
| Ad5 fiber | 401-581 (GenBank accession no. AP_000226) | 177 |
| knob | | |
| Ad5 penton | 1-571 (GenBank accession no. AP_000206) | 178 |
| Ad5 hexon | 1-952 (GenBank accession no. AP_000211) | 179 |
| Ad5 fiber | 1-43 (GenBank accession no. AP_000226) | 180 |
| tail | | |

TABLE 21
Ad35 Genomic Sequences
Ad35 Genomic Sequences
Reference Ad5 Genome Sequence: GenBank
accession no. AY128640 (SEQ ID NO: 209)

| | Exemplary Sequence | |
|---|---|---|
| Component | (position in reference) | SEQ ID NO: |
| Ad35 5′ (left) ITR | 1-137 | 181 |
| Ad35 3′ (right) ITR | 34658-34794 | 182 |
| Ad35 Packaging | 138-479 | 183 |
| Sequence | | |
| Ad35 E1 | 480-3400 | 184 |
| Ad35 E2 | 3966-23415 | 185 |
| Ad35 E3 | 27198-30622 | 186 |
| Ad35 fiber | 30826-31797 | 187 |
| Ad35 fiber tail | 30826-30951 | 188 |
| Ad35 fiber shaft | 30952-31224 | 189 |
| Ad35 fiber knob | 31225-31797 | 190 |
| Ad35 penton | 13690-15375 | 191 |
| Ad35 hexon | 18255-21113 | 192 |

TABLE 22
Ad35 Amino Acid Sequences
Ad35 Amino Acid Sequences

| | Exemplary Sequence | SEQ |
|---|---|---|
| Component | (position in reference) | ID NO: |
| Ad35 fiber | 1-323 (GenBank accession no. AP_000601) | 193 |
| Ad35 fiber | 43-133 (GenBank accession no. AP_000601) | 194 |
| shaft | | |
| Ad35 fiber | 134-323 (GenBank accession no. AP_000601) | 195 |
| knob | | |
| Ad35 penton | 1-561 (GenBank accession no. AP_000580) | 196 |
| Ad35 hexon | 1-952 (GenBank accession no. AP_000585) | 197 |
| Ad35 fiber | 1-42 (GenBank accession no. AP_000601) | 198 |
| tail | | |

In various embodiments, an Ad3, 5, 7, 11, 14, 16, 21, 34, 35, 37, or 50 vector or genome includes modifications that reduce and/or eliminate replication of the virus in recipients. Broadly, there are three recognized “generations” of adenoviral vectors and genomes engineered to reduce and/or eliminate replication of the virus in recipients. Adenoviral vectors of the present disclosure can include vectors according to any of these three generations.

In various embodiments, an Ad3, 5, 7, 11, 14, 16, 21, 34, 35, 37, or 50 genome differs from a reference Ad sequence (e.g., one or more canonical, representative, exemplary, or wild-type sequence of an adenovirus of a serotype of interest) at least in that the regulatory E1 gene (E1a and E1b) is removed from the Ad genome (“first generation” vector modifications). First generation Ad vector including an E1 deletion are an example of an E1-deleted vector. E1a and E1b are the first transcriptional regulatory factors produced during the adenoviral replication cycle. E1 deletion reduces or eliminates expression of certain viral genes controlled by E1, and E1-deleted helper viruses are replication-defective. Thus, first generation Ad vectors are deficient for replication in a recipient. In some embodiments, first-generation adenoviral vectors are engineered to remove E1 and E3 genes. Retained portions of the reference genome can be identical in sequence to a reference genome or can have less than 100% identity with a reference genome, e.g., at least 99%, 98%, 97%, 96%, 95%, 90%, 85%, 80%, or 75% identity. Without these E1 (or E1 and E3) genes, adenoviral vectors cannot replicate on their own but can be produced in mammalian cell lines that express E1 (e.g., of the same serotype) or another protein sufficient to restore expression of the certain viral genes. For illustration, where an E1-deficient Ad5 vector encodes an Ad5 E4orf6, the helper vector can be propagated in a cell line that expresses Ad5 E1. In one exemplary cell type for adenoviral vector production, HEK293 cells express Ad5 E1b55k, which is known to form a complex with Ad5 E4 protein ORF6.

In various embodiments, an Ad3, 5, 7, 11, 14, 16, 21, 34, 35, 37, or 50 genome differs from a reference Ad sequence at least in that the E1 gene (E1a and E1b) and one or more of non-structural genes E2, E3 and/or E4 are deleted (“second generation” modifications). Second generation Ads have greater payload capacity than first generation Ads and are more deficient for replication than first generation viruses. In some embodiments, second-generation adenoviral vectors, in addition to E1/E3 removal, are engineered to remove non-structural genes E2 and E4, resulting in increased capacity and reduced immunogenicity. Retained portions of the reference genome can be identical in sequence to a reference genome or can have less than 100% identity with a reference genome, e.g., at least 99%, 98%, 97%, 96%, 95%, 90%, 85%, 80%, or 75% identity.

In various embodiments, an Ad3, 5, 7, 11, 14, 16, 21, 34, 35, 37, or 50 genome differs from a reference Ad sequence at least in that they are engineered to remove all viral coding sequences from the Ad genome, and retain only the ITRs of the genome and the packaging sequence of the genome or a functional fragment thereof (“third generation” modifications). Third generation adenoviral vectors can also be referred to as gutless, high capacity adenoviral vectors, or helper-dependent adenoviral vectors (HdAds). Retained portions of the reference genome can be identical in sequence to a reference genome or can have less than 100% identity with a reference genome, e.g., at least 99%, 98%, 97%, 96%, 95%, 90%, 85%, 80%, or 75% identity.

Because third generation Ad genomes do not encode the proteins necessary for viral production, they are helper-dependent: a helper-dependent genome can only be packaged into a vector if they are present in a cell that includes a nucleic acid sequence that provides viral proteins in trans. These helper-dependent vectors are also characterized by still greater capacity than first and second generation vectors and decreased immunogenicity. Because HDAd vectors do not express viral genes when used as a vector, the risk of cytotoxicity or interferon response in recipients is reduced.

Helper-dependent adenoviral vectors (HDAd) engineered to lack all viral coding sequences can efficiently transduce a wide variety of cell types, and can mediate long-term transgene expression with negligible chronic toxicity. By deleting the viral coding sequences and leaving only the cis-acting elements necessary for genome replication (ITRs) and packaging (W), cellular immune response against the Ad vector is reduced. HDAd vectors have a large cloning capacity of up to allowing for the delivery of large payloads. These payloads can include large therapeutic genes or even multiple transgenes and large regulatory components to enhance, prolong, and regulate transgene expression. It has also been observed that the certain HDAd vector genomes can be most efficiently packaged when the genome has at least a minimum a total length, e.g., a minimum to total length of at least 20 kb (e.g., 20, 21, 22, 23, 24, 25, 26, 27, 28, 29, 30, 31, 32, 33, 34, or 35 kb) which length can include, e.g., a therapeutic payload and/or a “stuffer” sequence. Where a payload does not utilize a number of nucleotides that causes the adenoviral genome to have at least a target length, a stuffer sequence can be used to achieve or surpass the target length. The present disclosure includes that a minimum length for efficient packaging is not required for beneficial use of vectors provided herein, such that meeting any target length may be advantageous but not required for use of compositions and methods provided herein. Like other adenoviral vectors, typical HDAd genomes generally remain episomal and do not integrate with a host genome.

Because HDAd vectors do not encode the viral proteins required to produce viral particles, viral proteins must be provided in trans, e.g., expressed in and/or by cells in which the HDAd genome is present. In some HDAd vector systems, one viral genome (a helper genome) encodes all of the proteins (e.g., all of the structural viral proteins) required for replication but has a conditional defect in the packaging sequence, making it less likely to be packaged into a vector under certain vector production conditions (e.g., in the presence of an agent that reduces function of the conditionally defective packaging sequence). Thus, the HDAd donor viral genome includes (e.g., only includes) Ad ITRs, a payload (e.g., a therapeutic payload), and a functional packaging sequence (e.g., a wild-type packaging sequence or a functional fragment thereof), which allows the HDAd donor viral genome to be selectively packaged into HDAd viral vectors produced from structural components expressed from the helper vector genome. In other words, Ad3, 5, 7, 11, 14, 16, 21, 34, 35, 37, or 50 helper vectors can be used for production of Ad3, 5, 7, 11, 14, 16, 21, 34, 35, 37, or 50 donor vectors. Production of HD Ad3, 5, 7, 11, 14, 16, 21, 34, 35, 37, or 50 vectors can include co-transfection of a plasmid containing the HDAd vector genome and a packaging-defective helper virus that provides structural and non-structural viral proteins. The helper virus genome can rescue propagation of the Ad3, 5, 7, 11, 14, 16, 21, 34, 35, 37, or 50 donor vector and Ad3, 5, 7, 11, 14, 16, 21, 34, 35, 37, or 50 donor vector can be produced, e.g., at a large scale, and isolated. Various protocols are known in the art, e.g., at Palmer et al., 2009 Gene Therapy Protocols. Methods in Molecular Biology, Volume 433. Humana Press; Totowa, NJ: 2009. pp. 33-53. In some embodiments, a helper genome is E1-deficient.

In some HDAd vector systems, a helper genome utilizes a recombinase system (e.g., a Cre/loxP system) for conditional packaging. In certain such HDAd vector systems, a helper genome can include a packaging sequence or functional fragment thereof (e.g., a fragment of the packaging sequence that is sufficient for packaging, required for packaging, or required for efficient packaging of the Ad genome into the capsid) flanked by recombinase (e.g., loxP) sites so that contact with a corresponding recombinase (e.g., Cre recombinase) excises the packaging sequence or functional fragment thereof from the helper genome by recombinase-mediated (e.g., Cre-mediated) site-specific recombination between the recombinase sites (e.g., loxP sites). The present disclosure includes, among other things, Ad3, 5, 7, 11, 14, 16, 21, 34, 35, 37, or 50 helper vectors and genomes that include two recombination sites that flank a packaging sequence or functional fragment thereof, where the two recombination sites are sites corresponding to (i.e., for, or acted upon by) the same recombinase.

In various embodiments, a helper genome can include deletion of E1, e.g., where the helper genome includes all of the viral genes except for E1, as E1 expression products can be supplied by complementary expression from the genome of a producer cell line. In some embodiments, to prevent generation of replication competent Ad (RCA) as a consequence of homologous recombination between the helper and HDAd donor genomes present in producer cells, a “stuffer” sequence can be inserted into the E3 region to render any recombinants too large to be packaged and/or efficiently packaged.

For production of HDAd vectors, an HDAd donor genome can be delivered to cells that express a recombinase for excision of the conditional packaging sequence of a helper vector (e.g., 293 cells (HEK293) that expresses Cre recombinase), optionally where the HDAd donor genome is delivered to the cells in a non-viral vector form, such as a bacterial plasmid form (e.g., where the HDAd donor genome is present in a bacterial plasmid (pHDAd) and/or is liberated by restriction enzyme digestion). The same cells can be transduced with the helper genome including a packaging sequence or functional fragment thereof flanked by recombinase sites (e.g., loxP sites). Thus, producer cells can be transfected with the HDAd donor genome and transduced with a helper genome bearing a packaging sequence or a functional fragment thereof flanked by recombinase sites (e.g., loxP sites), where the cells express a recombinase (e.g., Cre) corresponding to the recombinase sites such that excision of the packaging sequence or functional fragment thereof renders the helper virus genome deficient for packaging (e.g., unpackageable), but still able to provide all of the necessary trans-acting factors for production of HDAd donor vector including the HDAd donor genome.

Similar HDAd production systems have been developed using FLP (e.g., FLPe)/frt site-specific recombination, where FLP-mediated recombination between frt sites flanking the packaging sequence or functional fragment thereof of the helper genome reduces or eliminates packaging of helper genomes in producer cells that express FLP.

HDAd vectors including the donor vector genome including the payload can be isolated from the producer cells. HDAd donor vectors can be further purified from helper vectors by physical means. In general, some contamination of helper vectors and/or helper genomes in HDAd viral vectors and HDAd viral vector formulations can occur and can be tolerated.

HDAd3, 5, 7, 11, 14, 16, 21, 34, 35, 37, and 50 donor vectors, donor genomes, helper vectors, and helper genomes are also exemplary of compositions provided herein and can be used in various methods of the present disclosure. An HDAd3, 5, 7, 11, 14, 16, 21, 34, 35, 37, or 50 vector or genome is a helper-dependent Ad3, 5, 7, 11, 14, 16, 21, 34, 35, 37, or 50 vector or genome. An Ad3, 5, 7, 11, 14, 16, 21, 34, 35, 37, or 50 helper vector is a vector that includes a helper genome that includes a conditionally expressed (e.g., frt-site or loxP-site flanked) packaging sequence or fragment thereof and encodes all of the necessary trans-acting factors for production of Ad3, 5, 7, 11, 14, 16, 21, 34, 35, 37, or 50 virions into which the donor genome can be packaged.

The present disclosure further includes an HDAd3, 5, 7, 11, 14, 16, 21, 34, 35, 37, or 50 donor vector production system including a cell including an HDAd3, 5, 7, 11, 14, 16, 21, 34, 35, 37, or 50 donor genome and an Ad3, 5, 7, 11, 14, 16, 21, 34, 35, 37, or 50 helper genome. In certain such cells, viral proteins encoded and expressed by the helper genome can be utilized in production of HDAd3, 5, 7, 11, 14, 16, 21, 34, 35, 37, or 50 donor vectors in which the HDAd3, 5, 7, 11, 14, 16, 21, 34, 35, 37, or 50 donor genome is packaged. Accordingly, the present disclosure includes methods of production of HDAd3, 5, 7, 11, 14, 16, 21, 34, 35, 37, or 50 donor vectors by culturing cells that include an HDAd3, 5, 7, 11, 14, 16, 21, 34, 35, 37, or 50 donor genome and an Ad3, 5, 7, 11, 14, 16, 21, 34, 35, 37, or 50 helper genome. In some embodiments, the cells encode and express a recombinase that corresponds to recombinase direct repeats that flank a packaging sequence of the Ad3, 5, 7, 11, 14, 16, 21, 34, 35, 37, or 50 helper vector. In some embodiments, the flanked packaging sequence of the Ad3, 5, 7, 11, 14, 16, 21, 34, 35, 37, or 50 helper genome has been excised.

In some embodiments, the Ad3, 5, 7, 11, 14, 16, 21, 34, 35, 37, or 50 helper genome encodes all Ad3, 5, 7, 11, 14, 16, 21, 34, 35, 37, or 50 coding sequences. In some embodiments, the Ad3, 5, 7, 11, 14, 16, 21, 34, 35, 37, or 50 helper genome encodes and/or expresses all Ad3, 5, 7, 11, 14, 16, 21, 34, 35, 37, or 50 coding sequences except for one or more coding sequences of E1 and/or an E3 coding sequence and/or an E4 coding sequence. In various embodiments, a helper genome that does not encode and/or express an Ad3, 5, 7, 11, 14, 16, 21, 34, 35, 37, or 50 E1 gene does not encode and/or express an Ad3, 5, 7, 11, 14, 16, 21, 34, 35, 37, or 50 E4 gene. In various embodiments, as will be appreciate by those of skill in the art, cells of compositions and methods for production of HDAd donor vectors can be cells that express an E1 expression product.

The present disclosure includes, among other things, HDAd3, 5, 7, 11, 14, 16, 21, 34, 35, 37, or 50 donor vectors and genomes that include Ad3, 5, 7, 11, 14, 16, 21, 34, 35, 37, or 50 ITRs (a 5′ Ad3, 5, 7, 11, 14, 16, 21, 34, 35, 37, or 50 ITR and a 3′ ITR of the same serotype), e.g., where two Ad3, 5, 7, 11, 14, 16, 21, 34, 35, 37, or 50 ITRs flank a packaging sequence and a payload. The present disclosure includes, among other things, HDAd3, 5, 7, 11, 14, 16, 21, 34, 35, 37, or 50 donor vectors and genomes in which E1 or a fragment thereof is deleted. The present disclosure includes, among other things, HDAd3, 5, 7, 11, 14, 16, 21, 34, 35, 37, or 50 vectors and genomes in which E3 or a fragment thereof is deleted.

In various embodiments, excision of a packaging sequence or functional fragment thereof from an Ad3, 5, 7, 11, 14, 16, 21, 34, 35, 37, or 50 helper genome reduces propagation of the vector by, e.g., at least 20%, 30%, 40%, 50%, 60%, 70%, 80%, 90%, 91%, 92%, 93%, 94%, 95%, 96%, 97%, 98%, 99%, 99.5%, 99.9%, or 100% (e.g., reduces propagation of the vector by a percentage having a lower bound of 20%, 30%, 40%, 50%, 60%, 70%, and an upper bound of 60%, 70%, 80%, 90%, 91%, 92%, 93%, 94%, 95%, 96%, 97%, 98%, 99%, 99.5%, 99.9% or 100%), optionally where percent propagation is measured as the number of viral particles produced by propagation of excised vector (vector from which the recombinase site-flanked sequence has been excised) as compared to complete vector (vector from which the recombinase site-flanked sequence has not been excised) or as compared to wild-type Ad3, 5, 7, 11, 14, 16, 21, 34, 35, 37, or 50 vector under comparable conditions.

An additional optional engineering consideration can be engineering of a helper genome having a size that permits separation of helper vector from HDAd3, 5, 7, 11, 14, 16, 21, 34, 35, 37, or 50 donor vector by centrifugation, e.g., by CsCl ultracentrifugation. One means of achieving this result is to increase the size of the helper genome as compared to a typical Ad3, 5, 7, 11, 14, 16, 21, 34, 35, 37, or 50 genome. In particular, adenoviral genomes can be increased by engineering to at least 104% of wild-type length. Certain helper vectors of the present disclosure can accommodate a payload and/or stuffer sequence.

The present disclosure includes that in various embodiments a vector or genome of the present disclosure can include a selection of components each selected from, or having at least 75% sequence identity (e.g., at least 75%, 80%, 85%, 90%, 95%, 96%, 97%, 98%, 99%, or 100% identity) to, a corresponding sequence of a single particular serotype. To provide an illustrative example, all components can correspond to (e.g., have at least 75% sequence identity to sequences of) Ad34, excepting sequences otherwise indicated (e.g., a payload, e.g., a heterologous payload).

In various embodiments, a vector of the present disclosure is an HDAd5/35 vector that includes Ad5 capsid proteins except that the fibers are chimeric in that they include an Ad5 fiber tail, an Ad35 fiber shaft, and an Ad35 fiber knob (see, e.g., Shayakhmetov et al. 2000 J. Virol 74(6):2567-2583), optionally where the Ad35 fiber knob is mutated for increased affinity to CD46 (e.g., Ad5/35++). In particular embodiments, an Ad5/35++ vector is a chimeric Ad5/35 vector with a mutant Ad35++ fiber knob (see, e.g., Wang et al. 2008 J. Virol. 82(21):10567-79, which is incorporated herein by reference in its entirety and particularly with respect to fiber knob mutations). In various embodiments, an Ad35++ mutant fiber knob is an Ad35 fiber knob mutated to increase the affinity to CD46, e.g., by 25-fold, e.g., such that the Ad35++ mutant fiber knob increases cell transduction efficiency, e.g., at lower multiplicity of infection (MOI) (Li and Lieber, FEBS Letters, 593(24): 3623-3648, 2019). In certain embodiments, an adenoviral vector is a chimeric “F35” vector in which all proteins are Ad5 proteins except that the fibers are chimeric in that they include an Ad5 fiber tail, an Ad35 fiber shaft, and an Ad35 fiber knob (e.g., as described in Shayakhmetov et al. 2000 J. Virol 74(6):2567-2583), where the Ad35 fiber knob is a mutant Ad35 fiber knob including mutations D207G and T245A causing increased affinity to CD46 (see, e.g., Wang et al. 2008 J. Virol. 82(21):10567-79), and optionally where the genome encoding the Ad5/35 vector includes an E1 deletion.

In various embodiments, an adenoviral vector or genome of the present disclosure can be an adenoviral vector and/or genome disclosed in WO 2021/003432, which is herein incorporated by reference in its entirety, and particularly with respect to adenoviral vectors and genomes.

### I(C). Ad3, 5, 7, 11, 14, 16, 21, 34, 35, 37, and 50 Gene Therapy Vector Payloads

Ad3, 5, 7, 11, 14, 16, 21, 34, 35, 37, or 50 donor vectors and genomes of the present disclosure can include a variety of heterologous nucleic acid payloads that can include any of one or more coding sequences that encode one or more expression products, one or more regulatory sequences operably linked to a coding sequence, one or more stuffer sequences, and the like. In various embodiments, the payload is engineered in order to achieve a desired result such as a therapeutic effect in a host cell or system, e.g., expression of a protein of therapeutic interest or of expression of a gene editing system, e.g., a CRISPR/Cas system, base editing system, or prime editing system to generate a sequence modification of therapeutic interest, e.g., to correct a nucleic acid lesion.

In some embodiments, a payload can include a gene. A gene can include not only coding sequences but also regulatory regions such as promoters, enhancers, termination regions, locus control regions (LCRs), termination and polyadenylation signal elements, splicing signal elements, silencers, insulators, and the like. A gene can include introns and other DNA sequences spliced from an expressed mRNA transcript, along with variants resulting from alternative splice sites. Coding sequences can also include alternative synonymous codon usage as compared to a reference sequence, e.g., codon usage modified as compared to a reference in accordance with codon preference of a specific organism or target cell type.

A payload can include a single gene or multiple genes. A payload can include a single coding sequence or a plurality of coding sequences. A payload can include a single regulatory sequence or a plurality of regulatory sequences. A payload can include a plurality of coding sequences where the individual expression products of the coding sequences function together, e.g., as in the case of an endonuclease and a guide RNA, or independently, e.g., as two separate proteins that do not directly or indirectly bind. As will be appreciated by those of skill in the art, any payload or payload component (e.g., a payload-encoded expression product or regulatory sequence) that is not encoded by the reference wild-type Ad3, 5, 7, 11, 14, 16, 21, 34, 35, 37, or 50 genome can be referred to herein as a heterologous expression product.

For the avoidance of doubt, the present disclosure includes variants of amino acid and nucleic acid sequences provided herein. Variants include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein and nucleic acid sequences described or disclosed herein where the variant exhibits substantially similar or improved biological function.

### I(C)(i). Payload Expression Products

A payload of an adenoviral donor vector or adenoviral donor genome of the present disclosure can include one or more coding sequences that encode any of a variety of expression products. Exemplary expression products include proteins, including without limitation replacement therapy proteins for treatment of diseases or conditions characterized by low expression or activity of a biologically active protein as compared to a reference level. Exemplary expression products include CRISPR/Cas, base editor, and prime editor systems. Exemplary expression products include antibodies, CARs, and TCRs. Exemplary expression products include small RNAs. In various embodiments, integration of all or a portion of a donor vector payload into a host cell genome is not required in order for delivery to the target cell of a donor vector or genome to produce an intended or target effect, e.g., in certain instances in which the intended or target effect includes editing of the host cell genome by a CRISPR, base editor, or prime editor system. In various embodiments, integration of all or a portion of a donor vector payload is required or preferred in order for delivery to the target cell of a donor vector or genome to produce an intended or target effect, e.g., where expression of a payload-encoded expression product is desired in progeny cells of a transduced target cell. In various embodiments, a payload can include a nucleic acid sequence engineered for integration into a host cell genome (an “integration element”), e.g., by recombination or transposition.

A gene sequence encoding one or more therapeutic proteins can be readily prepared by synthetic or recombinant methods from the relevant amino acid sequence. In particular embodiments, the gene sequence encoding any of these sequences can also have one or more restriction enzyme sites at the 5′ and/or 3′ ends of the coding sequence in order to provide for easy excision and replacement of the gene sequence encoding the sequence with another gene sequence encoding a different sequence. In particular embodiments, the gene sequence encoding the sequences can be codon optimized for expression in mammalian cells.

Particular examples of therapeutic genes and/or expression products include γ-globin, Factor VIII, γC, JAK3, IL7RA, RAG1, RAG2, DCLRE1C, PRKDC, LIG4, NHEJ1, CD3D, CD3E, CD3Z, CD3G, PTPRC, ZAP70, LCK, AK2, ADA, PNP, WHN, CHD7, ORAI1, STIM1, CORO1A, CIITA, RFXANK, RFX5, RFXAP, RMRP, DKC1, TERT, TINF2, DCLRE1B, SLC46A1, a FANC family gene (e.g., FancA, FancB, FancC, FancD1 (BRCA2), FancD2, FancE, FancF, FancG, FancI, FancJ (BRIP1), FancL, FancM, FancN (PALB2), FancO (RAD51C), FancP (SLX4), FancQ (ERCC4), FancR (RAD51), FancS (BRCA1), FancT (UBE2T), FancU (XRCC2), FancV (MAD2L2), and FancW (RFWD3)), soluble CD40, CTLA, Fas L, an antibody (e.g., that specifically binds CD4, CD5, CD7, CD52, IL1, IL2, IL6, TNF, P53, PTPN22, or DRB1*1501/DQB1*0602), an antibody to TCR specifically present on autoreactive T cells, IL4, IL10, IL12, IL13, IL1Ra, sIL1RI, sIL1RII, sTNFRI, sTNFRII, globin family genes, WAS, phox, dystrophin, pyruvate kinase, CLN3, ABCD1, arylsulfatase A, SFTPB, SFTPC, NLX2.1, ABCA3, GATA1, ribosomal protein genes, TERT, TERC, DKC1, TINF2, CFTR, LRRK2, PARK2, PARK7, PINK1, SNCA, PSEN1, PSEN2, APP, SOD1, TDP43, FUS, ubiquilin 2, C9ORF72, and other therapeutic genes and/or expression products described herein.

A therapeutic gene can be selected to provide a therapeutically effective response against diseases related to red blood cells and clotting. In particular embodiments, the disease is a hemoglobinopathy like thalassemia, or a sickle cell disease/trait. The therapeutic gene may be, for example, a gene that induces or increases production of hemoglobin; induces or increases production of β-globin, γ-globin, or α-globin; or increases the availability of oxygen to cells in the body. The therapeutic gene may be, for example, HBB or CYB5R3. Exemplary effective treatments may, for example, increase blood cell counts, improve blood cell function, or increase oxygenation of cells in patients. In another particular embodiment, the disease is hemophilia. The therapeutic gene may be, for example, a gene that increases the production of coagulation/clotting factor VIII or coagulation/clotting factor IX, causes the production of normal versions of coagulation factor VIII or coagulation factor IX, a gene that reduces the production of antibodies to coagulation/clotting factor VIII or coagulation/clotting factor IX, or a gene that causes the proper formation of blood clots. Exemplary therapeutic genes include F8 and F9. Exemplary effective treatments may, for example, increase or induce the production of coagulation/clotting factors VIII and IX; improve the functioning of coagulation/clotting factors VIII and IX, or reduce clotting time in subjects.

In various embodiments of the present disclosure, a donor vector encodes a globin gene, where the globin protein encoded by the globin gene is selected from a γ-globin, a β-globin, and/or an α-globin. Globin genes of the present disclosure can include, e.g., one or more regulatory sequences such as a promoter operably linked to a nucleic acid sequence encoding a globin protein. As those of skill in the art will appreciate, each of γ-globin, β-globin, and/or α-globin is a component of fetal and/or adult hemoglobin and is therefore useful in various vectors disclosed herein.

In various embodiments, increasing expression of a globin protein can refer to any of one or more of (i) increasing the amount, concentration, or expression (e.g., transcription or translation of nucleic acids encoding) in a cell or system of globin protein having a particular sequence; (ii) increasing the amount, concentration, or expression (e.g., transcription or translation of nucleic acids encoding) in a cell or system of globin protein of a particular type (e.g., the total amount of all proteins that would be identified as γ-globin (or alternatively β-globin or α-globin) by those of skill in the art or as set forth in the present specification) without respect to the sequences of the proteins relative to each other; and/or (iii) expressing in a cell or system a heterologous globin protein, e.g., a globin protein not encoded by a host cell prior to gene therapy.

The following references describe particular exemplary sequences of functional globin genes. References 1-4 relate to α-type globin sequences and references 4-12 relate to β-type globin sequences (including β and γ globin sequences), which sequences are hereby incorporated by reference: (1) GenBank Accession No. Z84721 (Mar. 19, 1997); (2) GenBank Accession No. NM_000517 (Oct. 31, 2000); (3) Hardison et al., J. Mol. Biol. (1991) 222(2):233-249; (4) A Syllabus of Human Hemoglobin Variants (1996), by Titus et al., published by The Sickle Cell Anemia Foundation in Augusta, Ga. (available online at globin.cse.psu.edu); (5) GenBank Accession No. J00179 (Aug. 26, 1993) or U01317.1; (6) Tagle et al., Genomics (1992) 13(3):741-760; (7) Grovsfeld et al., Cell (1987) 51(6):975-985; (8) Li et al., Blood (1999) 93(7):2208-2216; (9) Gorman et al., J. Biol. Chem. (2000) 275(46):35914-35919; (10) Slightom et al., Cell (1980) 21(3):627-638; (11) Fritsch et al., Cell (1980) 19(4): 959-972; (12) Marotta et al., J. Biol. Chem. (1977) 252(14):5040-5053. For additional coding and non-coding regions of genes encoding globins see, for example, by Marotta et al., Prog. Nucleic Acid Res. Mol. Biol. 19, 165-175, 1976, Lawn et al., Cell 21 (3), 647-651, 1980, and Sadelain et al., PNAS.; 92:6728-6732, 1995. In some embodiments, a globin gene encodes a G16D gamma globin variant.

An exemplary amino acid sequence of hemoglobin subunit β is provided, for example, at NCBI Accession No. P68871. An exemplary amino acid sequence for β-globin is provided, for example, at NCBI Accession No. NP_000509.

In addition to therapeutic genes and/or gene products, the transgene can also encode for therapeutic molecules, such as checkpoint inhibitor reagents, chimeric antigen receptor molecules specific to one or more cancer antigens, and/or T-cell receptors specific to one or more cancer antigens.

As another example, a therapeutic gene can be selected to provide a therapeutically effective response against a lysosomal storage disorder. In particular embodiments, the lysosomal storage disorder is mucopolysaccharidosis (MPS), type I; MPS II or Hunter Syndrome; MPS III or Sanfilippo syndrome; MPS IV or Morquio syndrome; MPS V; MPS VI or Maroteaux-Lamy syndrome; MPS VII or sly syndrome; α-mannosidosis; β-mannosidosis; glycogen storage disease type I, also known as GSDI, von Gierke disease, or Tay Sachs; Pompe disease; Gaucher disease; or Fabry disease. The therapeutic gene may be, for example a gene encoding or inducing production of an enzyme, or that otherwise causes the degradation of mucopolysaccharides in lysosomes. Exemplary therapeutic genes include IDUA or iduronidase, IDS, GNS, HGSNAT, SGSH, NAGLU, GUSB, GALNS, GLB1, ARSB, and HYAL1. Exemplary effective genetic therapies for lysosomal storage disorders may, for example, encode or induce the production of enzymes responsible for the degradation of various substances in lysosomes; reduce, eliminate, prevent, or delay the swelling in various organs, including the head (e.g., Macrocephaly), the liver, spleen, tongue, or vocal cords; reduce fluid in the brain; reduce heart valve abnormalities; prevent or dilate narrowing airways and prevent related upper respiratory conditions like infections and sleep apnea; reduce, eliminate, prevent, or delay the destruction of neurons, and/or the associated symptoms.

As another example, a therapeutic gene can be selected to provide a therapeutically effective response against a hyperproliferative disease. In particular embodiments, the hyperproliferative disease is cancer. The therapeutic gene may be, for example, a tumor suppressor gene, a gene that induces apoptosis, a gene encoding an enzyme, a gene encoding an antibody, or a gene encoding a hormone. Exemplary therapeutic genes and gene products include (in addition to those listed elsewhere herein) 101F6, 123F2 (RASSF1), 53BP2, ab1, ABLI, ADP, aFGF, APC, ApoAI, ApoAIV, ApoE, ATM, BAI-1, BDNF, Beta*(BLU), bFGF, BLC1, BLC6, BRCA1, BRCA2, CBFA1, CBL, C-CAM, CNTF, COX-1, CSFIR, CTS-1, cytosine deaminase, DBCCR-1, DCC, Dp, DPC-4, E1A, E2F, EBRB2, erb, ERBA, ERBB, ETS1, ETS2, ETV6, Fab, FCC, FGF, FGR, FHIT, fins, FOX, FUS1, FYN, G-CSF, GDAIF, Gene 21 (NPRL2), Gene 26 (CACNA2D2), GM-CSF, GMF, gsp, HCR, HIC-1, HRAS, hst, IGF, IL-1, IL-2, IL-3, IL-5, IL-6, IL-7, IL-8, IL-9, IL-11, ING1, interferon α, interferon β, interferon γ, IRF-1, JUN, KRAS, LUCA-1 (HYAL1), LUCA-2 (HYAL2), LYN, MADH4, MADR2, MCC, mda7, MDM2, MEN-I, MEN-II, MLL, MMAC1, MYB, MYC, MYCL1, MYCN, neu, NF-1, NF-2, NGF, NOEY1, NOEY2, NRAS, NT3, NT5, OVCA1, p16, p21, p27, p57, p73, p300, PGS, PIM1, PL6, PML, PTEN, raf, Rap1A, ras, Rb, RB1, RET, rks-3, ScFv, scFV ras, SEM A3, SRC, TALI, TCL3, TFPI, thrombospondin, thymidine kinase, TNF, TP53, trk, T-VEC, VEGF, VHL, WT1, WT-1, YES, and zac1. Exemplary effective genetic therapies may suppress or eliminate tumors, result in a decreased number of cancer cells, reduced tumor size, slow or eliminate tumor growth, or alleviate symptoms caused by tumors.

As another example, a therapeutic gene can be selected to provide a therapeutically effective response against an infectious disease. In particular embodiments, the infectious disease is human immunodeficiency virus (HIV). The therapeutic gene may be, for example, a gene rendering immune cells resistant to HIV infection, or which enables immune cells to effectively neutralize the virus via immune reconstruction, polymorphisms of genes encoding proteins expressed by immune cells, genes advantageous for fighting infection that are not expressed in the patient, genes encoding an infectious agent, receptor or coreceptor; a gene encoding ligands for receptors or coreceptors; viral and cellular genes essential for viral replication including; a gene encoding ribozymes, antisense RNA, small interfering RNA (siRNA) or decoy RNA to block the actions of certain transcription factors; a gene encoding dominant negative viral proteins, intracellular antibodies, intrakines and suicide genes. Exemplary therapeutic genes and gene products include α2β1; αvρ3; αvρ5; αvβ63; BOB/GPR15; Bonzo/STRL-33/TYMSTR; CCR2; CCR3; CCR5; CCR8; CD4; CD46; CD55; CXCR4; aminopeptidase-N; HHV-7; ICAM; ICAM-1; PRR2/HveB; HveA; α-dystroglycan; LDLR/α2MR/LRP; PVR; PRR1/HveC; and laminin receptor. A therapeutically effective amount for the treatment of HIV, for example, may increase the immunity of a subject against HIV, ameliorate a symptom associated with AIDS or HIV, or induce an innate or adaptive immune response in a subject against HIV. An immune response against HIV may include antibody production and result in the prevention of AIDS and/or ameliorate a symptom of AIDS or HIV infection of the subject, or decrease or eliminate HIV infectivity and/or virulence.

### I(C)(i)(a). Binding Domain, Antibody, CAR, and TCR Payload Expression Products

The present disclosure includes payloads that can include sequences that encode any of a variety of binding domains. Sequences that encode binding domains can encode, for example, antibodies, chimeric antigen receptors, TCRs, or other binding polypeptides.

Antibodies and antibody fragments are exemplary of binding domains. The term “antibody” can refer to a polypeptide that includes one or more canonical immunoglobulin sequence elements sufficient to confer specific binding to a particular antigen (e.g., a heavy chain variable domain, a light chain variable domain, and/or one or more CDRs). Thus, the term antibody includes, without limitation, human antibodies, non-human antibodies, synthetic and/or engineered antibodies, fragments thereof, and agents including the same. Antibodies can be naturally occurring immunoglobulins (e.g., generated by an organism reacting to an antigen). Synthetic, non-naturally occurring, or engineered antibodies can be produced by recombinant engineering, chemical synthesis, or other artificial systems or methodologies known to those of skill in the art.

As is well known in the art, typical human immunoglobulins are approximately 150 kD tetrameric agents that include two identical heavy (H) chain polypeptides (about 50 kD each) and two identical light (L) chain polypeptides (about 25 kD each) that associate with each other to form a structure commonly referred to as a “Y-shaped” structure. Typically, each heavy chain includes a heavy chain variable domain (VH) and a heavy chain constant domain (CH). The heavy chain constant domain includes three CH domains: CH1, CH2 and CH3. A short region, known as the “switch”, connects the heavy chain variable and constant regions. The “hinge” connects CH2 and CH3 domains to the rest of the immunoglobulin. Each light chain includes a light chain variable domain (VL) and a light chain constant domain (CL), separated from one another by another “switch.” Each variable domain contains three hypervariable loops known as “complement determining regions” (CDR1, CDR2, and CDR3) and four somewhat invariant “framework” regions (FR1, FR2, FR3, and FR4). In each VH and VL, the three CDRs and four FRs are arranged from amino-terminus to carboxy-terminus in the following order: FR1, CDR1, FR2, CDR2, FR3, CDR3, and FR4. The variable regions of a heavy and/or a light chain are typically understood to provide a binding moiety that can interact with an antigen. Constant domains can mediate binding of an antibody to various immune system cells (e.g., effector cells and/or cells that mediate cytotoxicity), receptors, and elements of the complement system. Heavy and light chains are linked to one another by a single disulfide bond, and two other disulfide bonds connect the heavy chain hinge regions to one another, so that the dimers are connected to one another and the tetramer is formed. When natural immunoglobulins fold, the FR regions form the beta sheets that provide the structural framework for the domains, and the CDR loop regions from both the heavy and light chains are brought together in three-dimensional space so that they create a single hypervariable antigen binding site located at the tip of the Y structure.

In some embodiments, an antibody is polyclonal, monoclonal, monospecific, or multispecific antibodies (including bispecific antibodies). In some embodiments, an antibody includes at least one light chain monomer or dimer, at least one heavy chain monomer or dimer, at least one heavy chain-light chain dimer, or a tetramer that includes two heavy chain monomers and two light chain monomers. Moreover, the term “antibody” can include (unless otherwise stated or clear from context) any art-known constructs or formats utilizing antibody structural and/or functional features including without limitation intrabodies, domain antibodies, antibody mimetics, Zybodies®, Fab fragments, Fab′ fragments, F(ab′)2 fragments, Fd′ fragments, Fd fragments, isolated CDRs or sets thereof, single chain antibodies, single-chain Fvs (scFvs), disulfide-linked Fvs (sdFv), polypeptide-Fc fusions, single domain antibodies (e.g., shark single domain antibodies such as IgNAR or fragments thereof), cameloid antibodies, camelized antibodies, masked antibodies (e.g., Probodies®), affybodies, anti-idiotypic (anti-Id) antibodies (including, e.g., anti-anti-Id antibodies), Small Modular ImmunoPharmaceuticals (“SMIPs™”), single chain or Tandem diabodies (TandAb®), VHHs, Anticalins®, Nanobodies® minibodies, BiTE®s, ankyrin repeat proteins or DARPINs®, Avimers®, DARTs, TCR-like antibodies, Adnectins®, Affilins®, Trans-bodies®, Affibodies®, TrimerX®, MicroProteins, Fynomers®, Centyrins®, and KALBITOR®s, CARs, engineered TCRs, and antigen-binding fragments of any of the above.

In various embodiments, an antibody includes one or more structural elements recognized by those skilled in the art as a complementarity determining region (CDR) or variable domain. In some embodiments, an antibody can be a covalently modified (“conjugated”) antibody (e.g., an antibody that includes a polypeptide including one or more canonical immunoglobulin sequence elements sufficient to confer specific binding to a particular antigen, where the polypeptide is covalently linked with one or more of a therapeutic agent, a detectable moiety, another polypeptide, a glycan, or a polyethylene glycol molecule). In some embodiments, antibody sequence elements are humanized, primatized, chimeric, etc, as is known in the art.

An antibody including a heavy chain constant domain can be, without limitation, an antibody of any known class, including but not limited to, IgA, secretory IgA, IgG, IgE and IgM, based on heavy chain constant domain amino acid sequence (e.g., alpha (α), delta (δ), epsilon (ε), gamma (γ) and mu (μ)). IgG subclasses are also well known to those in the art and include but are not limited to human IgG1, IgG2, IgG3 and IgG4. “Isotype” refers to the Ab class or subclass (e.g., IgM or IgG1) that is encoded by the heavy chain constant region genes. As used herein, a “light chain” can be of a distinct type, e.g., kappa (κ) or lambda (λ), based on the amino acid sequence of the light chain constant domain. In some embodiments, an antibody has constant region sequences that are characteristic of mouse, rabbit, primate, or human immunoglobulins. Naturally-produced immunoglobulins are glycosylated, typically on the CH2 domain. As is known in the art, affinity and/or other binding attributes of Fc regions for Fc receptors can be modulated through glycosylation or other modification. In some embodiments, an antibody may lack a covalent modification (e.g., attachment of a glycan) that it would have if produced naturally. In some embodiments, antibodies produced and/or utilized in accordance with the present invention include glycosylated Fc domains, including Fc domains with modified or engineered such glycosylation.

The term “antibody fragment” can refer to a portion of an antibody or antibody agent as described herein, and typically refers to a portion that includes an antigen-binding portion or variable region thereof. An antibody fragment can be produced by any means. For example, in some embodiments, an antibody fragment can be enzymatically or chemically produced by fragmentation of an intact antibody or antibody agent. Alternatively, in some embodiments, an antibody fragment can be recombinantly produced (i.e., by expression of an engineered nucleic acid sequence. In some embodiments, an antibody fragment can be wholly or partially synthetically produced. In some embodiments, an antibody fragment (particularly an antigen-binding antibody fragment) can have a length of at least about 50, 60, 70, 80, 90, 100, 110, 120, 130, 140, 150, 160, 170, 180, 190 amino acids or more, in some embodiments at least about 200 amino acids.

In some instances, it is beneficial for the binding domain to be derived from the same species it will ultimately be used in. For example, for use in humans, it may be beneficial for the antigen binding domain to include a human antibody, humanized antibody, or a fragment or engineered form thereof. Antibodies from human origin or humanized antibodies have lowered or no immunogenicity in humans and have a lower number of non-immunogenic epitopes compared to non-human antibodies. Antibodies and their engineered fragments will generally be selected to have a reduced level or no antigenicity in human subjects.

In various embodiments, a payload can encode a binding agent that is a checkpoint inhibitor such as an antibody that specifically binds an immune checkpoint protein. A number of immune checkpoint inhibitors are known. Immune checkpoint inhibitors can include peptides, antibodies, nucleic acid molecules and small molecules. Examples of immune checkpoints include PD-1, PD-L1, lymphocyte activation gene-3 (LAG-3), and T cell immunoglobulin and mucin domain-containing molecule 3 (TIM-3).

The present disclosure further includes antibodies and other binding domains that bind CD4, CD5, CD7, CD52, etc.; antibodies; antibodies to IL1, IL2, IL6; an antibody to TCR specifically present on autoreactive T cells; IL4; IL10; IL12; IL13; IL1Ra; sIL1RI; sIL1RII; antibodies to TNF; ABCA3; ABCD1; ADA; AK2; APP; arginase; arylsulfatase A; A1AT; CD3D; CD3E; CD3G; CD3Z; CFTR; CHD7; chimeric antigen receptor (CAR); CIITA; CLN3; complement factor, CORO1A; CTLA; C1 inhibitor; C9ORF72; DCLRE1B; DCLRE1C; decoy receptors; DKC1; DRB1*1501/DQB1*0602; dystrophin; enzymes; Factor VIII, FANC family genes (FancA, FancB, FancC, FancD1 (BRCA2), FancD2, FancE, FancF, FancG, FancI, FancJ (BRIP1), FancL, FancM, FancN (PALB2), FancO (RAD51C), FancP (SLX4), FancQ (ERCC4), FancR (RAD51), FancS (BRCA1), FancT (UBE2T), FancU (XRCC2), FancV (MAD2L2), and FancW (RFWD3)); Fas L; FUS; GATA1; globin family genes (i.e., γ-globin); F8; glutaminase; HBA1; HBA2; HBB; IL7RA; JAK3; LCK; LIG4; LRRK2; NHEJ1; NLX2.1; neutralizing antibodies; ORAI1; PARK2; PARK7; phox; PINK1; PNP; PRKDC; PSEN1; PSEN2; PTPN22; PTPRC; P53; pyruvate kinase; RAG1; RAG2; RFXANK; RFXAP; RFX5; RMRP; ribosomal protein genes; SFTPB; SFTPC; SOD1; soluble CD40; STIM1; sTNFRI; sTNFRII; SLC46A1; SNCA; TDP43; TERT; TERC; TINF2; ubiquilin 2; WAS; WHN; ZAP70; γC; and other therapeutic genes described herein.

Particular types of hematopoietic cells (e.g., T cells) can be engineered to encode and/or express chimeric antigen receptor (CAR) constructs. CARs can include several distinct subcomponents that can cause cells to recognize and kill target cells such as cancer cells. The subcomponents include at least an extracellular component and an intracellular component.

An extracellular CAR component can include a binding domain that specifically binds a marker that is preferentially present on the surface of unwanted cells. When the binding domain binds such markers, the intracellular component directs a cell to destroy the bound cancer cell. The binding domain is typically a single-chain variable fragment (scFv) derived from a monoclonal antibody (mAb), but it can be based on other formats which include an antibody-like antigen binding site.

Intracellular CAR components provide activation signals based on the inclusion of an effector domain. First generation CARs utilized the cytoplasmic region of CD3ζ as an effector domain. Second generation CARs utilized CD3ζ in combination with cluster of differentiation 28 (CD28) or 4-1BB (CD137), while third generation CARs have utilized CD3ζ in combination with CD28 and 401BB within intracellular effector domains.

Intracellular or otherwise cytoplasmic signaling components of a CAR are responsible for activation of the cell in which the CAR is expressed. The term “intracellular signaling components” or “intracellular components” is thus meant to include any portion of the intracellular domain sufficient to transduce an activation signal. Intracellular components of expressed CAR can include effector domains. An effector domain is an intracellular portion of a fusion protein or receptor that can directly or indirectly promote a biological or physiological response in a cell when receiving the appropriate signal. In certain embodiments, an effector domain is part of a protein or protein complex that receives a signal when bound, or it binds directly to a target molecule, which triggers a signal from the effector domain. An effector domain may directly promote a cellular response when it contains one or more signaling domains or motifs, such as an immunoreceptor tyrosine-based activation motif (ITAM). In other embodiments, an effector domain will indirectly promote a cellular response by associating with one or more other proteins that directly promote a cellular response, such as co-stimulatory domains.

Effector domains can provide for activation of at least one function of a modified cell upon binding to the cellular marker expressed by a cancer cell. Activation of the modified cell can include one or more of differentiation, proliferation and/or activation or other effector functions. In particular embodiments, an effector domain can include an intracellular signaling component including a T cell receptor and a co-stimulatory domain which can include the cytoplasmic sequence from a co-receptor or co-stimulatory molecule.

An effector domain can include one, two, three or more receptor signaling domains, intracellular signaling components (e.g., cytoplasmic signaling sequences), co-stimulatory domains, or combinations thereof. Exemplary effector domains include signaling and stimulatory domains selected from: 4-1BB (CD137), CARD11, CD3γ, CD3δ, CD3ε, CD3ζ, CD27, CD28, CD79A, CD79B, DAP10, FcRα, FcRβ (FcεR1b), FcRγ, Fyn, HVEM (LIGHTR), ICOS, LAG3, LAT, Lck, LRP, NKG2D, NOTCH1, pTα, PTCH2, OX40, ROR2, Ryk, SLAMF1, Slp76, TCRα, TCRβ, TRIM, Wnt, Zap70, or any combination thereof. In particular embodiments, exemplary effector domains include signaling and co-stimulatory domains selected from: CD86, FcγRIIa, DAP12, CD30, CD40, PD-1, lymphocyte function-associated antigen-1 (LFA-1), CD2, CD7, LIGHT, NKG2C, B7-H3, a ligand that specifically binds with CD83, CDS, ICAM-1, GITR, BAFFR, SLAMF7, NKp80 (KLRF1), CD127, CD160, CD19, CD4, CD8α, CD8β, IL2Rβ, IL2Rγ, IL7Rα, ITGA4, VLA1, CD49a, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), PSGL1, CD100 (SEMA4D), CD69, SLAMF6 (NTB-A, Ly108), SLAM (CD150, IPO-3), BLAME (SLAMF8), SELPLG (CD162), LTBR, GADS, PAG/Cbp, NKp44, NKp30, or NKp46.

Intracellular signaling component sequences that act in a stimulatory manner may include ITAMs. Examples of ITAMs including primary cytoplasmic signaling sequences include those derived from CD3γ, CD3δ, CD3ε, CD3ζ, CD5, CD22, CD66d, CD79a, CD79b, and common FcRγ (FCER1G), FcγR11a, FcRβ (Fcε Rib), DAP10, and DAP12. In particular embodiments, variants of CD3ζ retain at least one, two, three, or all ITAM regions.

In particular embodiments, an effector domain includes a cytoplasmic portion that associates with a cytoplasmic signaling protein, where the cytoplasmic signaling protein is a lymphocyte receptor or signaling domain thereof, a protein including a plurality of ITAMs, a co-stimulatory domain, or any combination thereof.

Additional examples of intracellular signaling components include the cytoplasmic sequences of the CD3ζ chain, and/or co-receptors that act in concert to initiate signal transduction following binding domain engagement.

A co-stimulatory domain is domain whose activation can be required for an efficient lymphocyte response to cellular marker binding. Some molecules are interchangeable as intracellular signaling components or co-stimulatory domains. Examples of costimulatory domains include CD27, CD28, 4-1BB (CD 137), OX40, CD30, CD40, PD-1, ICOS, lymphocyte function-associated antigen-1 (LFA-1), CD2, CD7, LIGHT, NKG2C, B7-H3, and a ligand that specifically binds with CD83. For example, CD27 co-stimulation has been demonstrated to enhance expansion, effector function, and survival of human CART cells in vitro and augments human T cell persistence and anti-cancer activity in vivo (Song et al. Blood. 2012; 119(3):696-706). Further examples of such co-stimulatory domain molecules include CDS, ICAM-1, GITR, BAFFR, HVEM (LIGHTR), SLAMF7, NKp80 (KLRF1), NKp44, NKp30, NKp46, CD160, CD19, CD4, CD8α, CD8β, IL2Rβ, IL2Rγ, IL7Rα, ITGA4, VLA1, CD49a, ITGA4, IA4, CD49D, ITGA6, VLA-6, CD49f, ITGAD, CD11d, ITGAE, CD103, ITGAL, CD11a, ITGAM, CD11b, ITGAX, CD111c, ITGB1, CD29, ITGB2, CD18, ITGB7, TNFR2, TRANCE/RANKL, DNAM1 (CD226), SLAMF4 (CD244, 2B4), CD84, CD96 (Tactile), NKG2D, CEACAM1, CRTAM, Ly9 (CD229), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, and CD19a.

In particular embodiments, the amino acid sequence of the intracellular signaling component includes a variant of CD3ζ and a portion of the 4-1BB intracellular signaling component.

In particular embodiments, the intracellular signaling component includes (i) all or a portion of the signaling domain of CD3ζ, (ii) all or a portion of the signaling domain of 4-1BB, or (iii) all or a portion of the signaling domain of CD3ζ and 4-1BB.

Intracellular components may also include one or more of a protein of a Wnt signaling pathway (e.g., LRP, Ryk, or ROR2), NOTCH signaling pathway (e.g., NOTCH1, NOTCH2, NOTCH3, or NOTCH4), Hedgehog signaling pathway (e.g., PTCH or SMO), receptor tyrosine kinases (RTKs) (e.g., epidermal growth factor (EGF) receptor family, fibroblast growth factor (FGF) receptor family, hepatocyte growth factor (HGF) receptor family, insulin receptor (IR) family, platelet-derived growth factor (PDGF) receptor family, vascular endothelial growth factor (VEGF) receptor family, tropomycin receptor kinase (Trk) receptor family, ephrin (Eph) receptor family, AXL receptor family, leukocyte tyrosine kinase (LTK) receptor family, tyrosine kinase with immunoglobulin-like and EGF-like domains 1 (TIE) receptor family, receptor tyrosine kinase-like orphan (ROR) receptor family, discoidin domain (DDR) receptor family, rearranged during transfection (RET) receptor family, tyrosine-protein kinase-like (PTK7) receptor family, related to receptor tyrosine kinase (RYK) receptor family, or muscle specific kinase (MuSK) receptor family); G-protein-coupled receptors, GPCRs (Frizzled or Smoothened); serine/threonine kinase receptors (BMPR or TGFR); or cytokine receptors (IL1R, IL2R, IL7R, or IL15R).

CAR generally also include one or more linker sequences that are used for a variety of purposes within the molecule. For example, a transmembrane domain can be used to link the extracellular component of the CAR to the intracellular component. A flexible linker sequence often referred to as a spacer region that is membrane-proximal to the binding domain can be used to create additional distance between a binding domain and the cellular membrane. This can be beneficial to reduce steric hindrance to binding based on proximity to the membrane. A common spacer region used for this purpose is the IgG4 linker. More compact spacers or longer spacers can be used, depending on the targeted cell marker. Other potential CAR subcomponents are described in more detail elsewhere herein. Components of CAR are now described in additional detail as follows: (a) Binding Domains; (b) Intracellular Signalling Components; (c) Linkers; (d) Transmembrane Domains; (e) Junction Amino Acids; and (f) Control Features Including Tag Cassettes.

Transmembrane domains within a CAR molecule, often serve to connect the extracellular component and intracellular component through the cell membrane. The transmembrane domain can anchor the expressed molecule in the modified cell's membrane.

The transmembrane domain can be derived either from a natural and/or a synthetic source. When the source is natural, the transmembrane domain can be derived from any membrane-bound or transmembrane protein. Transmembrane domains can include at least the transmembrane region(s) of the α, β or ζ chain of a T-cell receptor, CD28, CD27, CD3 epsilon, CD45, CD4, CD5, CD8, CD9, CD16, CD22; CD33, CD37, CD64, CD80, CD86, CD134, CD137 and CD154. In particular embodiments, a transmembrane domain may include at least the transmembrane region(s) of, e.g., KIRDS2, OX40, CD2, CD27, LFA-1 (CD 11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2Rβ, IL2Rγ, IL7R a, ITGA1, VLA1, CD49a, ITGA4, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, TNFR2, DNAM1(CD226), SLAMF4 (CD244, 2B4), CD84, CD96 (Tactile), CEACAM1, CRT AM, Ly9(CD229), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, or NKG2C. In particular embodiments, a variety of human hinges can be employed as well including the human Ig (immunoglobulin) hinge (e.g., an IgG4 hinge, an IgD hinge), a GS linker (e.g., a GS linker described herein), a KIR2DS2 hinge or a CD8a hinge.

TCRs refer to naturally occurring T cell receptors. Payloads of the present disclosure can encode a TCR or a CAR/TCR hybrids that includes an element of a TCR and an element of a CAR. For example, a CAR/TCR hybrid could have a naturally occurring TCR binding domain with an effector domain that the TCR binding domain is not naturally associated with. A CAR/TCR hybrid could have a mutated TCR binding domain and an ITAM signaling domain. A CAR/TCR hybrid could have a naturally occurring TCR with an inserted non-naturally occurring spacer region or transmembrane domain.

### I(C)(i)(b). Gene Editing Systems and Components

In various embodiments, a payload of the present disclosure encodes at least one component, or all components, of a gene editing system. Gene editing systems of the present disclosure include CRISPR systems, base editing, and prime editing systems. Broadly, gene editing systems can include a plurality of components including a gene editing enzyme selected from a CRISPR-associated RNA-guided endonuclease, a base editing enzyme, and a prime editing enzyme and at least one gRNA. Accordingly, gene editing systems of the present disclosure can include either (i) in the case of a CRISPR system, a CRISPR enzyme that is a CRISPR-associated RNA-guided endonuclease and at least one guide RNA (gRNA), (ii) in the case of a base editing system, a base editing enzyme and at least one gRNA, or (iii) in the case of a prime editing system and at least one prime editing gRNA. Nucleotide sequences encoding gene editing systems as disclosed herein are typically too large for inclusion in many limited-capacity vector systems, but the large capacity of adenoviral vectors permits inclusion of such sequences in adenoviral vectors and genomes of the present disclosure. An additional advantage of adenoviral vectors and genomes with payloads encoding gene editing systems or components of the present disclosure is that adenoviral genomes do not naturally integrate into host cell genomes, which facilitates transient expression of gene editing systems and components, which can be desirable, e.g., to avoid immunogenicity and/or genotoxicity.

In other embodiments, a gene editing system can include engineered zing finger nucleases (ZFN). For instance, a ZFN is an artificial endonuclease that consists of a designed zinc finger protein (ZFP) fused to the cleavage domain of the FokI restriction enzyme. A ZFN may be redesigned to cleave new targets by developing ZFPs with new sequence specificities. For genome engineering, a ZFN is targeted to cleave a chosen genomic sequence. The cleavage event induced by the ZFN provokes cellular repair processes that in turn mediate efficient modification of the targeted locus. If the ZFN-induced cleavage event is resolved via non-homologous end joining, this can result in small deletions or insertions, effectively leading to gene knockout. If the break is resolved via a homology-based process in the presence of an investigator-provided donor, small changes or entire transgenes can be transferred, often without selection, into the chromosome; which can be referred to as ‘gene correction’ and ‘gene addition,’ respectively.

In some embodiments, a gene editing system (e.g., a CRISPR system, base editing system, or prime editing system) is engineered to modify a nucleic acid sequence that encodes γ-globin, e.g., to increase expression of γ-globin. The main fetal form of hemoglobin, hemoglobin F (HbF) is formed by pairing of γ-globin polypeptide subunits with α-globin polypeptide subunits. Human fetal γ-globin genes (HBG1 and HBG2; two highly homologous genes produced by evolutionary duplication) are ordinarily silenced around birth, while expression of adult β-globin gene expression (HBB and HBD) increases. Mutations that cause or permit persistent expression of fetal γ-globin throughout life can ameliorate phenotypes of β-globin deficiencies. Thus, reactivation of fetal γ-globin genes can be therapeutically beneficial, particularly in subjects with β-globin deficiency. A variety of mutations that cause increased expression of γ-globin are known in the art (see, e.g., Wienert, Trends in Genetics 34(12): 927-940, 2018, which is incorporated herein by reference in its entirety and with respect to mutations that increase expression of γ-globin). Certain such mutations are found in the HBG1 promoter or HBG2 promoter.

In various embodiments, a gene editing system designed to increase expression of γ-globin includes an HBG1/2 promoter-targeted gRNA that is designed to increase expression of γ-globin coding by modification and/or inactivation of a BCL11A repressor protein binding site. In various embodiments, a gene editing system designed to increase expression of γ-globin includes a bcl11a-targeted gRNA that is designed to increase expression of γ-globin by modification and/or inactivation of the erythroid bcl11a enhancer to reduce BCL11A repressor protein expression in erythroid cells. In various embodiments, a gene editing system designed to increase expression of γ-globin includes a gRNA targeted to cause a loss of function mutation in the gene encoding BCL11A.

### I(C)(i)(b)(1). CRISPR Payload Expression Products

The present disclosure includes, among other things, CRISPR editing agents and systems, and nucleic acids encoding the same, e.g., where the nucleic acid is present in an adenoviral vector or genome. A CRISPR editing system can include a CRISPR editing enzyme and/or at least one gRNA as components thereof. The CRISPR (Clustered Regularly Interspaced Short Palindromic Repeats)/Cas (CRISPR-associated protein) nuclease system is an engineered nuclease system used for genetic engineering that is based on a bacterial system. It is based in part on the adaptive immune response of many bacteria and archaea. When a virus or plasmid invades a bacterium, segments of the invader's DNA are converted into CRISPR RNAs (crRNA) by the bacteria's “immune” response. The crRNA then associates, through a region of partial complementarity, with another type of RNA called tracrRNA to guide a Cas nuclease to a region homologous to the crRNA in the target DNA called a “protospacer.” The Cas nuclease cleaves the DNA to generate blunt ends at the double-strand break at sites specified by a 20-nucleotide complementary strand sequence contained within the crRNA transcript. In some instances, the Cas nuclease requires both the crRNA and the tracrRNA for site-specific DNA recognition and cleavage.

Guide RNAs (gRNAs) are an example of an element that can target CRISPR editing. In its simplest form, gRNA provides a sequence that targets a site within a genome based on complementarity (e.g., crRNA). As explained below, however, gRNA can also include additional components. For example, in particular embodiments, gRNA can include a targeting sequence (e.g., crRNA) and a component to link the targeting sequence to a cutting element. This linking component can be tracrRNA. In particular embodiments, gRNA including crRNA and tracrRNA can be expressed as a single molecule referred to as single gRNA (sgRNA). gRNA can also be linked to a cutting element through other mechanisms such as through a nanoparticle or through expression or construction of a dual or multi-purpose molecule. Those of skill in the art will appreciate that gRNA or other targeting elements that can be used to generate a selected nucleic acid sequence correction or modification, e.g., in a host cell of an adenoviral donor vector or genome of the present disclosure, can be readily designed and implemented, e.g., based on available sequence information.

In particular embodiments, targeting elements (e.g., gRNA) can include one or more modifications (e.g., a base modification, a backbone modification), to provide the nucleic acid with a new or enhanced feature (e.g., improved stability). Modified backbones may include those that retain a phosphorus atom in the backbone and those that do not have a phosphorus atom in the backbone. Suitable modified backbones containing a phosphorus atom may include, for example, phosphorothioates, chiral phosphorothioates, phosphorodithioates, phosphotriesters, aminoalkylphosphotriesters, methyl and other alkyl phosphonates such as 3′-alkylene phosphonates, 5′-alkylene phosphonates, chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity where one or more internucleotide linkages is a 3′ to 3′, a 5′ to 5′ or a 2′ to 2′ linkage. Suitable targeting elements having inverted polarity can include a single 3′ to 3′ linkage at the 3′-most internucleotide linkage (i.e. a single inverted nucleoside residue in which the nucleobase is missing or has a hydroxyl group in place thereof). Various salts (e.g., potassium chloride or sodium chloride), mixed salts, and free acid forms can also be included.

Examples of cutting elements include nucleases. CRISPR-Cas loci have more than 50 gene families and there are no strictly universal genes, indicating fast evolution and extreme diversity of loci architecture. Exemplary Cas nucleases include Cas1, Cas1B, Cas2, Cas3, Cas4, Cas5, Cas6, Cas7, Cas8, Cas9 (also known as Csn1 or Csx12; including, e.g., spCas9, dCas9, nCas9, and Cas9-SpRY), Cas10, Cas12 (e.g., Cas12a (e.g., LbCas12a, AsCas12a, FnCas12a, MB3Cas12a, Cas12a-M11, Cas12a-M13 (e.g., Cas12a-M13-1), Cas12a-M26 (e.g., Cas12a-M26-1), Cas12a-M28 (e.g., Cas12a-M28-1), Cas12a-M29 (e.g., Cas12a-M29-1), Cas12a-M30 (e.g., Cas12a-M30-1), Cas12a-M31 (e.g., Cas12a-M31-1), Cas12a-M32 (e.g., Cas12a-M32-1), Cas12a-M57, Cas12a-M58, Cas12a-M59, Cas12a-M60 (e.g., Cas12a-M60-9), Cas12a-M61, or Cas12a-M62), Cas12b, Cas12c, Cas12g, Cas12h, or Cas12i), Cas-Phi, CasX, CasY, Cpf1, C2c3, C2c2, C2c1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10O, Csx16, CsaX, Csx3, Csx1, Csx15, Csf1, Csf2, Csf3, Csf4, and variants thereof.

There are three main types of Cas nucleases (type I, type II, and type III), and 10 subtypes including 5 type I, 3 type II, and 2 type III proteins (see, e.g., Hochstrasser and Doudna, Trends Biochem Sci, 2015:40(1):58-66). Type II Cas nucleases include Cas1, Cas2, Csn2, and Cas9. These Cas nucleases are known to those skilled in the art. For example, the amino acid sequence of the Streptococcus pyogenes wild-type Cas9 polypeptide is set forth, e.g., in NCBI accession no. NP_269215, and the amino acid sequence of Streptococcus thermophilus wild-type Cas9 polypeptide is set forth, e.g., in NCBI accession no. WP_011681470.

In particular embodiments, Cas9 refers to an RNA-guided double-stranded DNA-binding nuclease protein or nickase protein. Wild-type Cas9 nuclease has two functional domains, e.g., RuvC and HNH, that cut different DNA strands. Cas9 can induce double-strand breaks in genomic DNA (target DNA) when both functional domains are active. The Cas9 enzyme, in some embodiments, includes one or more catalytic domains of a Cas9 protein derived from bacteria such as Corynebacter, Sutterella, Legionella, Treponema, Filif actor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the Cas9 is a fusion protein, e.g. the two catalytic domains are derived from different bacterial species.

In some embodiments, crRNA and tracrRNA can be combined into one molecule called a single gRNA (sgRNA). In this engineered approach, the sgRNA guides Cas to target any desired sequence (see, e.g., Jinek et al., Science 337:816-821, 2012; Jinek et al., eLife 2:e00471, 2013; Segal, eLife 2:e00563, 2013). Thus, the CRISPR/Cas system can be engineered to create a double-strand break at a desired target in a genome of a cell, and harness the cell's endogenous mechanisms to repair the induced break by HDR, or NHEJ. Particular embodiments described herein utilize homology arms to promote HDR at defined integration sites.

In various embodiments, variants of the Cas9 nuclease include a single inactive catalytic domain, such as a RuvC″ or HNH″ enzyme or a nickase. A Cas9 nickase has only one active functional domain and, in some embodiments, cuts only one strand of the target DNA, thereby creating a single strand break or nick. In some embodiments, the mutant Cas9 nuclease having at least a D10A mutation is a Cas9 nickase. In other embodiments, the mutant Cas9 nuclease having at least a H840A mutation is a Cas9 nickase. Other examples of mutations present in a Cas9 nickase include N854A and N863 A. A double-strand break is introduced using a Cas9 nickase if at least two DNA-targeting RNAs that target opposite DNA strands are used. A double-nicked induced double-strand break is repaired by HDR or NHEJ. This gene editing strategy generally favors HDR and decreases the frequency of indel mutations at off-target DNA sites. The Cas9 nuclease or nickase, in some embodiments, is codon-optimized for the target cell or target organism.

### I(C)(i)(b)(2). Base Editor Payload Expression Products

The present disclosure includes, among other things, base editing agents and and systems, and nucleic acids encoding the same, e.g., where the nucleic acid is present in an adenoviral vector or genome. A base editing system can include a base editing enzyme and/or at least one gRNA as components thereof. A base editing system can utilize a deaminase (e.g., a base editing system) for editing of nucleic acid targets. In certain particular embodiments, a base editing agent and/or a base editing system of the present disclosure is present in an Ad3, 5, 7, 11, 14, 16, 21, 34, 35, 37, or 50 adenoviral vector

Deamination is the removal of an amine group from a molecule such as a nucleotide of a nucleic acid. Deamination of a nucleotide can cause changes in the sequence of a nucleic acid, and deaminases are useful in editing for at least that reason. Deamination of adenosine (A) yields inosine (I), which has the same base pairing preferences as a guanosine in DNA and is thus recognized by cell replication machinery as guanosine, resulting in an A-T to G-C transition. Deamination of cytosine (C) yields uridine (U), which is recognized by cell replication machinery as thymine, resulting in a C-G to T-A transition. Collectively, cytosine and adenosine deamination can be used to cause transitions from A to G, T to C, C to T, or G to A. Other deaminase activities are also known. For example, deamination of 5-methylcytosine yields thymine and deamination of guanosine yields xanthine, though xanthine, like guanosine, pairs with cytosine. Deaminases that deaminate cytosine can be referred to as cytosine deaminases. Deaminases that deaminate adenosine can be referred to as adenosine deaminases.

In particular embodiments, a base editing enzyme includes a cytidine deaminase domain or an adenine deaminase domain. Certain embodiments utilize a cytidine deaminase domain as the nucleobase deaminase enzyme. Particular embodiments utilize an adenine deaminase domain as the nucleobase deaminase enzyme.

Examples of cytosine deaminase enzymes (CBEs) include APOBEC1, APOBEC3A, APOBEC3G, CDA1, and AID. APOBEC1 particularly accepts single-stranded (ss)DNA as a substrate but is incapable of acting on double-stranded (ds)DNA.

For adenosine base editors (ABEs), exemplary adenosine deaminases that can act on DNA for adenine base editing include a mutant TadA adenosine deaminases (TadA*) that accepts DNA as its substrate. E. coli TadA typically acts as a homodimer to deaminate adenosine in transfer RNA (tRNA). TadA* deaminase catalyzes the conversion of a target ‘A’ to ‘I’ (inosine), which is treated as ‘G’ by cellular polymerases. Subsequently, an original genomic A-T base pair can be converted to a G-C pair. As the cellular inosine excision repair is not as active as uracil excision, ABE does not require any additional inhibitor protein like UGI in CBE. In some embodiments, an ABE can include one or more, or all, of three components including a wild-type E. coli tRNA-specific adenosine deaminase (TadA) monomer, which can play a structural role during base editing, a TadA* mutant TadA monomer that catalyzes deoxyadenosine deamination, and/or a Cas nickase such as Cas9(D10A). In certain embodiments, there is a linker positioned between TadA and TadA*, and in certain embodiments there is a linker positioned between TadA* and the Cas nickase. In various embodiments, one or both linkers includes at least 6 amino acids, e.g., at least 5, 6, 7, 8, 9, 10, 15, 20, 25, 30, 35, 40, 45, or 50 amino acids (e.g., having a lower bound of 5, 6, 7, 8, 9, 10, or 15, amino acids and an upper bound of 20, 25, 30, 35, 40, 45, or 50 amino acids). In various embodiments, one or both linkers include 32 amino acids. In some embodiments, one or both linkers has a sequence according to (SGGS)₂-XTEN-(SGGS)₂ (SEQ ID NO: 213) or a sequence otherwise known to those of skill in the art.

In various embodiments, an editing system includes a deaminase associated with a DNA binding domain such as a catalytically impaired nuclease domain. In various embodiments, the DNA binding domain can localize the deaminase to a target nucleic acid in which one or more nucleotides are deaminated by the deaminase. Catalytically impaired nuclease domains are polypeptide domains that have amino acid sequences engineered from reference nuclease domain sequences but that have a reduced ability to cause double-strand breaks (DSBs) as compared to the reference (e.g., a wild type and/or fully functional nuclease) or have no ability to cause double-strand breaks. As referred to herein, a nickase refers to a catalytically impaired nuclease domain that, upon contact with a double-stranded nucleic acid substrate, cleaves one strand (e.g., a target strand) of the double-stranded nucleic acid but not both strands of the double-stranded nucleic acid. In various embodiments, a nickase, upon contact with a double-stranded nucleic acid substrate, cleaves one strand of the double-stranded nucleic acid but not both strands of the double-stranded nucleic acid in at least 70% of contacted double-stranded nucleic acid substrates (e.g., at least 70%, 75%, 80%, 85%, 90%, 91%, 92%, 93%, 94%, 95%, 96%, 97%, 98%, 99%, or 100% of double-stranded nucleic acid substrates).

Base editing systems are exemplary of editing systems that include deaminase enzymes. A base editing enzyme includes a deaminase enzyme fused to a DNA binding domain that is a catalytically impaired nuclease domain (e.g., a nickase, e.g., a nickase that nicks a single strand, e.g., a non-edited strand). DNA binding domains of base editing enzymes can be RNA guided DNA binding domains, in that an RNA guide can direct the DNA binding domain to a target nucleic acid sequence. Catalytically impaired nuclease domains of a base editing enzyme can bind nucleic acids and can localize the deaminase enzyme to a target nucleic acid.

Any nuclease of the CRISPR system can be engineered to produce a catalytically impaired nuclease domain (e.g., a nickase) and used within a base editing enzyme or system. Exemplary Cas nucleases include Cas1, Cas1B, Cas2, Cas3, Cas4, Cas5, Cas6, Cas7, Cas8, Cas9 (also known as Csn1 or Csx12; including, e.g., spCas9, dCas9, nCas9, and Cas9-SpRY), Cas10, Cas12 (e.g., Cas12a (e.g., LbCas12a, AsCas12a, FnCas12a, MB3Cas12a, Cas12a-M11, Cas12a-M13 (e.g., Cas12a-M13-1), Cas12a-M26 (e.g., Cas12a-M26-1), Cas12a-M28 (e.g., Cas12a-M28-1), Cas12a-M29 (e.g., Cas12a-M29-1), Cas12a-M30 (e.g., Cas12a-M30-1), Cas12a-M31 (e.g., Cas12a-M31-1), Cas12a-M32 (e.g., Cas12a-M32-1), Cas12a-M57, Cas12a-M58, Cas12a-M59, Cas12a-M60 (e.g., Cas12a-M60-9), Cas12a-M61, or Cas12a-M62), Cas12b, Cas12c, Cas12g, Cas12h, or Cas12i), Cas-Phi, CasX, C2c3, C2c2, C2c1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csx1, Csx15, Csf1, Csf2, Csf3, Csf4, and variants thereof. Numerous forms and variants of Cas nucleases are known in the art (e.g., spCas9, dCas9, nCas9, Cas9-SpRY, and Cas12a) and can have distinct characteristics, including for example recognition of distinct PAMs and PAM positions.

In various embodiments, a catalytically impaired nuclease domain generates a single-stranded nick in the non-deaminated DNA strand, inducing cells to repair the non-deaminated strand using the deaminated strand as a template. To provide one example, nCas9 can create a nick in target DNA by cutting a single strand, reducing the likelihood of detrimental indel formation as compared to methods that require a double-strand break.

Particular embodiments utilize a nuclease-inactive Cas9 (dCas9) as the catalytically disabled nuclease. However, any nuclease of the CRISPR system (many of which are described above) can be disabled and used within a base editing system. In particular embodiments, a Cas9 domain with high fidelity is selected where the Cas9 domain displays decreased electrostatic interactions between the Cas9 domain and a sugar-phosphate backbone of a DNA, as compared to a wild-type Cas9 domain. In some embodiments, a Cas9 domain (e.g., a wild type Cas9 domain) includes one or more mutations that decrease the association between the Cas9 domain and a sugar-phosphate backbone of a DNA. Cas9 domains with high fidelity are known to those skilled in the art. For example, Cas9 domains with high fidelity have been described in Kleinstiver (2016 Nature 529: 490-495) and Slaymaker (2015 Science 351: 84-88).

Other DNA binding nucleases can also be used in a base editing enzyme. For example, base-editing systems can utilize zinc finger nucleases (ZFNs) (see, e.g., Urnov 2010 Nat Rev Genet. 11(9): 636-46) and transcription activator like effector nucleases (TALENs) (see, e.g., Joung 2013 Nat Rev Mol Cell Biol. 14(1): 49-55). For additional information regarding DNA-binding nucleases, see, e.g., US 2018/0312825.

In various embodiments, a base editing enzyme includes a DNA glycosylase inhibitor. A DNA glycosylase inhibitor can override natural DNA repair mechanisms that might otherwise repair the intended base editing. A DNA glycosylase inhibitor can be a uracil DNA glycosylase inhibitor protein (UGI). One exemplary UGI is described in Wang (1991 Gene 99:31-37). In particular embodiments, a base editing enzyme can include one or more DNA glycosylase inhibitor domains (e.g., UGI domains). In various embodiments, base editing enzymes that include more than one DNA glycosylase inhibitor domain (e.g., UGI domain) can generate fewer indels and/or deaminate target nucleic acids more efficiently than base editing enzymes that includes one DNA glycosylase inhibitor domain (e.g., UGI domain) and/or no DNA glycosylase inhibitor domains (e.g., UGI domains). For example, in particular embodiments, dCas9 or a Cas9 nickase can be fused to a cytidine deaminase domain and the dCas9 or Cas9 nickase can be fused to one or more UGI domains.

In particular embodiments, a deaminase domain is associated with the N-terminus of a catalytically disabled nuclease. In particular embodiments, a deaminase domain is associated with the N-terminus of a catalytically disabled nuclease. In certain embodiments, one or more glycosylase inhibitors (e.g., UGI domain) can be associated with the C-terminus of a catalytically disabled nuclease.

Components of base editors can be fused directly (e.g., by direct covalent bond) or via linkers. For example, the catalytically disabled nuclease can be fused via a linker to the deaminase enzyme and/or a glycosylase inhibitor. Multiple glycosylase inhibitors can also be fused via linkers. As will be understood by one of ordinary skill in the art, linkers can be used to link any peptides or portions thereof.

Exemplary linkers include polymeric linkers (e.g., polyethylene, polyethylene glycol, polyamide, polyester); amino acid linkers; carbon-nitrogen bond amide linkers; cyclic or acyclic, substituted or unsubstituted, branched or unbranched aliphatic or heteroaliphatic linkers; monomeric, dimeric, or polymeric aminoalkanoic acid linkers; aminoalkanoic acid (e.g., glycine, ethanoic acid, alanine, β-alanine, 3-aminopropanoic acid, 4-aminobutanoic acid, 5-pentanoic acid) linkers; monomeric, dimeric, or polymeric aminohexanoic acid (Ahx) linkers; carbocyclic moiety (e.g., cyclopentane, cyclohexane) linkers; aryl or heteroaryl moiety linkers; and phenyl ring linkers.

Linkers can also include functionalized moieties to facilitate attachment of a nucleophile (e.g., thiol, amino) from a peptide to the linker. Any electrophile may be used as part of the linker. Exemplary electrophiles include activated esters, activated amides, Michael acceptors, alkyl halides, aryl halides, acyl halides, and isothiocyanates.

In particular embodiments, linkers range from 4-100 amino acids in length. In particular embodiments, linkers are 4 amino acids, 9 amino acids, 14 amino acids, 16 amino acids, 32 amino acids, or 100 amino acids.

Various base editing enzymes are known in the art. Examples of base editing enzymes include BE1 (APOBEC1-16 amino acid (aa) linker-Sp dCas9 (D10A, H840A) (see, e.g., Komor 2016 Nature 533: 420-424)), BE2 (APOBEC1-16aa linker-Sp dCas9 (D10A, H840A)-4aa linker-UGI (see, e.g., Komor 2016 Nature 533: 420-424)), BE3 (APOBEC1-16aa linker-SpnCas9 (D10A)-4aa linker-UGI (see, e.g., Komor 2016 Nature 533: 420-424)), HF-BE2 (rAPOBEC1-HF2 nCas9-UGI), HF-BE3 (APOBEC1-16aa linker-HF nCas9 (D10A)-4aa linker-UGI (see, e.g., Rees 2017 Nat. Commun. 8: 15790)), BE4 (rAPOBEC1-Sp nCas9-UGI-UGI), BE4max (APOBEC1-32aa linker-Sp nCas9 (D10A)-9aa linker-UGI-9aa linker-UGI (see, e.g., Koblan 2018 Nat. Biotechnol 36(9): 843-846 and/or Komor 2017 Sci. Adv. 3(8): eaao4774)), BE4-GAM (Gam-16aa linker-APOBEC1-32aa linker-Sp nCas9 (D10A)-9aa linker-UGI-9aa linker-UGI (see, e.g., Komor 2017 Sci. Adv. 3(8): eaao4774)), YE1-BE3 (APOBEC1 (W90Y, R126E)-16aa linker-Sp nCas9 (D10A)-4aa linker-UGI (see, e.g., Kim 2017 Nat. Biotechnol. 35: 475-480)), EE-BE3 (APOBEC1 (R126E, R132E)-16aa linker-Sp nCas9 (D10A)-4aa linker-UGI (see, e.g., Kim 2017 Nat. Biotechnol. 35: 475-480)), YE2-BE3 (APOBEC1 (W90Y, R132E)-16aa linker-Sp nCas9 (D10A)-4aa linker-UGI (see, e.g., Kim 2017 Nat. Biotechnol. 35: 475-480)), YEE-BE3 (APOBEC1 (W90Y, R126E, R132E)-16aa linker-Sp nCas9 (D10A)-4aa linker-UGI (see, e.g., Kim 2017 Nat. Biotechnol. 35: 475-480)), VQR-BE3 (APOBEC1-16aa linker-Sp VQR nCas9 (D10A)-4aa linker-UGI (see, e.g., Kim 2017 Nat. Biotechnol. 35: 475-480)), EQR-BE3 (rAPOBEC1-EQR SpnCas9-UGI), VRER-BE3 (APOBEC1-16aa linker-Sp VRER nCas9 (D10A)-4aa linker-UGI (see, e.g., Kim 2017 Nat. Biotechnol. 35: 475-480)), Sa-BE3 (APOBEC1-16aa linker-Sa nCas9 (D10A)-4aa linker-UGI (see, e.g., Kim 2017 Nat. Biotechnol. 35: 475-480)), SA-BE4 (APOBEC1-32aa linker-Sa nCas9 (D10A)-9aa linker-UGI-9aa linker-UGI (see, e.g., Komor 2017 Sci. Adv. 3(8): eaao4774)), SaBE4-Gam (Gam-16aa linker-APOBEC1-32aa linker-Sa nCas9 (D10A)-9aa linker-UGI-9aa linker-UGI (see, e.g., Komor 2017 Sci. Adv. 3(8): eaao4774)), SaKKH-BE3 (APOBEC1-16aa linker-Sa KKH nCas9 (D10A)-4aa linker-UGI (see, e.g., Kim 2017 Nat. Biotechnol. 35: 475-480)), FNLS-BE3 (rAPOBEC1-Sp nCas9-UGI), RA-BE3 (rAPOBEC1 (RA)-Sp nCas9-UGI), Cas12a-BE (APOBEC1-16aa linker-dCas12a-14aa linker-UGI (see, e.g., Li 2018 Nat. Biotechnol. 36: 324-327)), Target-AID (Sp nCas9 (D10A)-100aa linker-CDA1-9aa linker-UGI (see, e.g., Nishida 2016 Science 353(6305): aaf8729)), Target-AID-NG (Sp nCas9 (D10A)-NG-100aa linker-CDA1-9aa linker-UGI (see, e.g., Nishimasu 2018 Science 361(6408): 1259-1262)), xBE3 (APOBEC1-16aa linker-xCas9(D10A)-4aa linker-UGI (see, e.g., Hu 2018 Nature 556: 57-63)), eA3A-BE3 (APOBEC3A (N37G)-16aa linker-Sp nCas9(D10A)-4aa linker-UGI (see, e.g., Gehrke 2018 Nat. Biotechnol. 36(10): 977-982)), A3A-BE3 (hAPOBEC3A-16aa linker-Sp nCas9(D10A)-4aa linker-UGI (see, e.g., Wang 2018 Nat. Biotechnol. 36: 946-949)), eA3A-HF1-BE3-2xUGI (APOBEC3A-HF1 Sp nCas9-UGI-UGI), eA3A-HypaBE3-2xUGI (APOBEC3A-Hypa Sp nCas9-UGI-UGI), hA3A-BE3 (hAPOBEC3A-Sp nCas9-UGI), hA3B-BE3 (hAPOBEC3B-Sp nCas9-UGI), hA3G-BE3 (hAPOBEC3G-Sp nCas9-UGI), hAID-BE3 (hAPOBEC3A-Sp nCas9-UGI), SaCas9-BE3 (rAPOBEC1-SanCas9-UGI), xCas9-BE3 (rAPOBEC1-xnCas9-UGI), ScCas9-BE3 (rAPOBEC1-ScnCas9-UGI), SniperCas9-BE3 (rAPOBEC1-SnipernCas9-UGI), iSpyMac-BE3 (rAPOBEC1-iSpyMacnCas9-UGI), CRISPR-X (Sp dCas9-MS2-hAID), TAM (Sp dCas9-hAID (P182X)), AncBE4-Max (rAPOBEC1-Sp nCas9-UGI-UGI), ABE7.8/9/10 (ecTadA-ecTadA*-Sp nCas9), xCas9-ABE7.10 (ecTadA-ecTadA*-nxCas9), VQR-ABE (ecTadA-ecTadA*-Sp VQR nCas9), Sa(KKH)-ABE ecTadA-ecTadA*-Sa KKH nCas9), ABEmax (ecTadA-ecTadA*-Sp nCas9), ABE7.10max (ecTadA-ecTadA*-SpnCas9), ABE8e)ecTadA-ecTadA*-SpnCas9), PE1 (dSpCas9-MMLV-RT), PE2 (dSpCas9-MMLV-RT), PE3 (nSpCas9-MMLV-RT), and BE-PLUS (10× GCN4-Sp nCas9(D10A)/ScFv-rAPOBEC1-UGI (see, e.g., Jiang 2018 Cell Res. 28(8): 855-861)). For additional examples of BE complexes, including adenine deaminase base editors, see, e.g., Rees 2018 Nat. Rev Genet. 19(12): 770-788 and/or Kantor 2020 Int. J. Mol. Sci. 21(17): 6240.

Various base editors are “dual base editors” that can edit both adenine and cytosine. Dual base editor enzymes can be fusion polypeptides that include a cytosine deaminase domain and an adenine deaminase domain. For instance, a dual base editor known as Target-ACEmax includes a codon-optimized fusion of the cytosine deaminase PmCDA1, the adenosine deaminase TadA, and a Cas9 nickase (Target-ACEmax) (see, e.g., Sakata 2020 Nature Biotechnology, 38(7), 865-869). Other exemplary dual base editors include SPACE (synchronous programmable adenine and cytosine editor). The SPACE editing enzyme is a fusion polypeptide that includes both miniABEmax-V82G and Target-AID editing domains together with a Cas9 (SpCas9-D10A) nickase domain (see, e.g., Grünewald 2020 Nat. Biotechnol. 38:861-864). A dual base editor known as A&C-BEmax includes a fusion of both cytidine and adenosine deaminase domains with a Cas9 nickase domain (see, e.g., Zhang 2020 Nat. Biotechnol. 38:856-860).

A base editing system can include a guide RNA (gRNA) that includes at least a fragment that base pairs with a complementary target nucleic acid (e.g., at least 80% identity between the fragment and the complement of the target nucleic acid, e.g., 80%, 85%, 90%, 91%, 92%, 93%, 94%, 95%, 96%, 97%, 98%, 99%, or 100% identity), where the fragment can be 10 to 40 nucleotides in length (e.g., equal to or about 10, 15, 16, 17, 18, 19, 20, 21, 22, 23, 24, 25, 30, 35 or 40 nucleotides in length, e.g., 17-24 or 17-20 nucleotides in length), e.g., where the target sequence is upstream of an appropriate PAM site. In various embodiments, a fragment of a gRNA that is complementary to a target nucleic acid sequence is positioned at the 5′ end of a gRNA or is 5′ relative to one or more other fragments of the gRNA. In various embodiments, a gRNA includes a sequence that forms a stemloop structure and binds with and/or recruits the catalytically impaired nuclease domain of a base editing enzyme. A gRNA that includes both a fragment that base pairs with a complementary target nucleic acid sequence and a fragment that forms a stemloop structure and binds with and/or recruits the catalytically impaired nuclease domain of a base editing enzyme can be referred to as a single guide RNA (sgRNA). The fragments of sgRNA can be associated via a linker fragment.

A guide RNA (e.g., an sgRNA) is thought to randomly interrogate nucleic acids until it encounters a nucleic acid that is sufficiently complementary to the 5′ fragment. Upon binding of a gRNA to a DNA nucleic acid target present in double-stranded DNA, base pairing between the gRNA and target nucleic acid strand causes displacement of a small segment of single-stranded DNA. In various embodiments, the gRNA recruits the catalytically impaired nuclease domain. Nucleotides of the displaced single-stranded DNA can be modified by the deaminase enzyme. The resultant base pair can then be repaired by cellular mismatch repair machinery to a new base pair, or alternatively in some instances reverted by base excision repair mediated by uracil glycosylase. In various embodiments, a glycosylase inhibitor (e.g., UGI) reduces the occurrence of reversion.

The present disclosure includes base editing enzymes and systems engineered to increase the editing window of base editing. For example, the present disclosure includes circularly permuted base editors, described for example in Huang 2020 Nature Biotechnology, 37(6), 626-631, which is incorporated herein with respect to base editing enzymes, base editing systems, and editing windows thereof. Circularly permuted base editing enzymes and systems can be characterized by an increased range of target bases that can be modified within the protospacer up to and including, for example, at least 5, 6, 7, 8, or 9 nucleotides. For example, certain base editing systems including Cas9 variants, including cytosine and four adenine base editing enzymes, can deaminated nucleotides in a window expanded from about 4-5 nucleotides to up about 8-9 nucleotides, optionally with reduced byproduct formation.

Base editing enzymes and systems can also target and/or modify RNA molecules. One advantage of using RNA editing systems is that there is no permanent change in the genome. RNA base editors achieve analogous changes using components that base modify RNA. For example, adenosine deaminase can modify transcribed mRNA, replacing adenosine with inosine at a target site. In mammals, the most prevalent post-transcription RNA editing case is catalyzed by the adenosine deaminase enzymes (ADARs). ADAR proteins are a highly conserved family of proteins that include a single deaminase domain (DD) and one or more double-stranded RNA (dsRNA)-binding domains ADARs (e.g., ADAR 1 or ADAR2) bind to dsRNA and catalyzes adenosine to inosine (A-to-I), which is read as guanosine by cellular translational machinery. ADAR1 and ADAR2 domains have been demonstrated to achieve RNA editing, e.g., in HSCs (see, e.g., Harter 2009 Nat. Immunol. 10(1): 109-115). A number of catalytically inactive Cas proteins have also been used to target RNA molecules, including Cas9, Cas13a, Cas13b, and Cas13d.

REPAIR (RNA editing for programmable adenosine to inosine replacement) is an RNA base editing system that includes catalytically inactive Cas13 protein and the deaminase activity of ADAR2. Cas13 generally includes two HEPN (higher eukaryotes and prokaryotes nucleotide-binding) domains, which contribute to RNA-targeted nucleolytic activity. Mutations of HEPNs abolish RNA cleavage activity while maintaining RNA targeting activity, which has been used to create an RNA base editing enzyme (e.g., REPAIR) (see, e.g., Cox 2017 Science 358:1019-1027). dCas13-ADAR2_{DD} includes catalytically inactive dCas13 variant with RNA deaminase ADAR2 (E488Q), and can execute RNA editing for programmable A-to-I (G) replacement. RNA Editing for Specific C-to-U Exchange (RESCUE) was later developed (see, e.g., Abudayyeh 2019 Science 365:382-386). gRNAs for mRNA editing can include, e.g., a fragment complementary to a target RNA and an ADAR-recruiting fragment, such that site-directed RNA editing is achieved by recruiting ADAR to a complementary target nucleic acid. RNA-guided RNA-targeting CRISPR nuclease C2C2 (later named as Cas13a) from Leptotrichia shahii was illustrated (Abudayyeh 2016 Science 353: aaf5573).

Other examples of RNA editing systems that include ADARs can include removing the endogenous RNA-targeting domains (dsRBMS) from human adenosine deaminase and replacing them with an antisense RNA oligonucleotide to produce a recombinant enzyme that can be directed to edit a selected RNA target. In particular embodiments, an ADAR2 deaminase domain is fused with an RNA-binding protein, and the sequence bound by the RNA-binding protein is associated with an antisense RNA guide oligonucleotide. In various embodiments, the RNA-binding protein is derived from λ-phage N protein-boxB RNA interaction, which normally regulates antitermination during transcription of λ-phage mRNAs. λN peptide mediates binding of the N protein, is only 22 amino acids long, and the boxB RNA hairpin that it recognizes is only 17 nucleotides long and they can bind with nanomolar affinity. Thus, in various embodiments, λN peptide can be fused to the deaminase domain of human ADAR2 (λN-DD). In various embodiments, a mutant ADAR2_{DD}(E488Q) can be used as the deaminase domain. In various embodiments, an editing enzyme can include an ADAR deaminase domain and 2 or more λN domains (e.g., 2, 3, 4, 5, or 6 λN domains). Examples of such editing enzymes and systems are described, e.g., in Montiel-Gonzalez 2013 PNAS 110(45): 18285-18290 and Montiel-Gonzalez 2016 Nuc. Acids. Res. 44(2): e157, each of which is incorporated herein by reference with respect to editing systems.

Other examples of editing systems that include ADARs can include leveraging endogenous ADAR for programmable editing of RNA (LEAPER) editing system that employs short engineered ADAR-recruiting RNAs (arRNAs) to recruit native ADAR1 or ADAR2 deaminase enzymes to change a specific adenosine to inosine. For example, in certain particular embodiments, an ADAR protein or its catalytic domain can be fused with a λN peptide. In certain embodiments, an ADAR protein or its catalytic domain can be fused with a λN peptide and a SNAP-tag or a Cas protein (e.g., dCas13b). A gRNA can recruit the editing enzyme to the specific site. Further description of LEAPER editing systems can be found in Qu 2019 Nat. Biotech. 1059-1069, which is incorporated herein by reference with respect to LEAPER editing systems and

Base editing systems can cause point mutations without producing double-strand breaks. Base editing systems can cause point mutations without producing undesired insertions and deletions (indels). For example, a base editing system can cause indels in less than 10%, 9%, 8%, 7%, 6%, 5.5%, 5%, 4.5%, 4%, 3.5%, 3%, 2.5%, 2%, 1.5%, 1%, 0.5%, or 0.1% of edited cells or editing events.

Those of skill in the art will appreciate that a base editing gRNA (e.g., sgRNA) or other targeting elements to generate a selected nucleic acid sequence modification in a target nucleic acid can be readily designed and implemented, e.g., based on available sequence information.

Base editing systems do not require double-stranded DNA breaks. Base editing systems do not require a donor fragment or template. Base editing systems provide precise control of the site at which the editing system modifies a target nucleic acid. Base editing systems can be multiplexed to achieve editing of multiple targets using a single editing enzyme, optionally including therapeutic targets. The present disclosure includes base editing systems that include a plurality of sgRNAs (e.g., two or more, e.g., two, three, four, or five) sgRNAs.

### I(C)(i)(b)(3). Prime Editor Payload Expression Products

The present disclosure includes, among other things, prime editing agents and systems, and nucleic acids encoding the same, e.g., where the nucleic acid is present in an adenoviral vector or genome. A prime editing system can include a prime editing enzyme and/or at least one pegRNA as components thereof. Prime editing can introduce all possible types of point mutations, small insertions, and small deletions in a precise and targeted manner. A prime editing enzyme includes a reverse transcriptase fused to a DNA binding domain that is a catalytically impaired nuclease domain (e.g., a nickase, e.g., a nickase that nicks a single strand, e.g., a non-edited strand). A reverse transcriptase is an enzyme that can synthesize a DNA molecule from an RNA template. A reverse transcriptase generally produces a DNA molecule that is complementary to the RNA template.

In particular embodiments, an editing enzyme includes an AMV reverse transcriptase, MLV reverse transcriptase, HIV-1 reverse transcriptase, or bacterial reverse transcriptase. Certain embodiments utilize an MLV reverse transcriptase domain. Reverse transcriptases of the present disclosure can have wild type amino acid sequences or engineered amino acid sequences.

Examples of reverse transcriptase enzymes include AMV reverse transcriptases (e.g., wild type AMV reverse transcriptase (RNase H plus activity), eAMV™ (engineered; RNase Hplus activity) or THermoScript™ (engineered; reduce RNAase H activity)), MLV reverse transcriptases (e.g., wild type M-MLV reverse transcriptase, GoScript™, or MultiScribe™ (RNase H plus activity), AccuScript Hi-Fi (engineered, RNase H minus (3′-5′ exonuclease activity), Affinity Script (engineered; E69K/E302R/W313F/L435G/N454K; unspecified RNase H activity), ArrayScript™ (engineered; unspecified RNase H activity), BioScript™ (engineered; reduced RNase H activity), CycleScript™ (engineered), EnzScript™ (engineered; RNase H minus), EpiScript™ (engineered; RNase H minus), Expand™ reverse transcriptase (engineered; RNase H reduced), FIREScript (engineered; RNase H plus), GrandScript (engineered; RNase H plus), iScript™ (engineered; RNase H plus), Maxima™ RT (engineered; RNase H plus and minus), MonsterScript™ (engineered; RNase H minus), PrimeScript™ (engineered; RNase H minus), PrimeScript™ II (engineered; RNase H minus), PrimeScript™ III (engineered; RNase H minus), PrimeScript™ IV (engineered; RNase H minus), ProtoScript® (Engineered; RNase H plus), ProtoScript® II (engineered; RNase H reduced), qScript (engineered; RNase H plus), RevertAid™ (engineered; RNase H plus and minus), ReverTra Ace® (engineered; RNase H minus), RevertUp II™ (engineered; RNase H minus), Rocketscript™ (engineered; RNase H plus and minus), Script (engineered; RNase H minus), SMART® (engineered), SMARTScribe™ (engineered; unspecified RNase H activity), SuperScript™ II (engineered; 524G/D583N/E562Q; RNase H reduced), SuperScript™ III (engineered; 204R/V223H/T306K/F309N/D524G/D583N/E562Q; RNase H reduced), SuperScript™ IV (engineered; RNase H reduced), or Transcriptor reverse transcriptase (engineered; RNase H plus)), an HIV-1 reverse transcriptase (e.g., HIV-1 RT (wild type of group M subtype B; RNase H plus), Biotools high retrotranscriptase (engineered group O variant (K65R/V75I); RNase H plus), or Sunscript® (engineered group O variants with changes K358R/A359G/S360A; RNase H plus and minus)), a bacterial group II intron reverse transcriptase (e.g., Marathon RT (wild type (Eubacterium rectale); lacks RNase H domain) or TGIRT®-III RT (wild type (Geobacillus stearothermophilus); lacks RNase H domain), a bacterial DNA polymerase (e.g., BcaBEST polymerase (engineered (Bacillus caldotenax DNA polymerase without 5′-3′ and 3′-5′ exonuclease activity); lacks RNase H domain), Bst 3.0 DNA polymerase (G. stearothermophilus DNA polymerase I, large fragment; lacks 5′-3′ and 3′-5′ exonuclease activity; lacks RNase H domain), RapiDxFire™ reverse transcriptase (lacks RNase H domain), Volcano2G DNA polymerase (engineered Thermus aquaticus DNA polymerase; lacks RNase H domain), or Volcano3G DNA polymerase (engineered T. aquaticus DNA polymerase; lacks RNase H domain)), SOLIScript (engineered; RNase H reduced), Omniscript® (heterodimeric RT; RNase H plus), and SensiScript® (heterodimeric RT; RNase H plus).

In various embodiments, a reverse transcriptase is a retrovirus reverse transcriptase. In various embodiments, a reverse transcriptase is a murine leukemia virus (MLV) reverse transcriptase (RT) (e.g., an engineered MLV RT). In various embodiments, a reverse transcriptase is a bacterial group II intron RT.

In various embodiments, a prime editing enzyme or system includes a reverse transcriptase associated with a DNA binding domain such as a catalytically impaired nuclease domain. In various embodiments, the DNA binding domain can localize the reverse transcriptase to a target nucleic acid in which one or more nucleotides are substituted, inserted, and/or deleted.

DNA binding domains of prime editing enzymes can be RNA guided DNA binding domains, in that an RNA guide can direct the DNA binding domain to a target nucleic acid sequence. Catalytically impaired nuclease domains of a prime editing enzyme can bind nucleic acids and can localize the reverse transcriptase enzyme to a target nucleic acid in which one or more nucleotides are substituted, inserted, and/or deleted by the prime editing system.

Any nuclease of the CRISPR system can be engineered to produce a catalytically impaired nuclease domain (e.g., a nickase) and used within a prime editing enzyme or system. Exemplary Cas nucleases include Cas1, Cas1B, Cas2, Cas3, Cas4, Cas5, Cas6, Cas7, Cas8, Cas9 (also known as Csn1 or Csx12; including, e.g., spCas9, dCas9, nCas9, and Cas9-SpRY), Cas10, Cas12 (e.g., Cas12a (e.g., LbCas12a, AsCas12a, FnCas12a, MB3Cas12a, Cas12a-M11, Cas12a-M13 (e.g., Cas12a-M13-1), Cas12a-M26 (e.g., Cas12a-M26-1), Cas12a-M28 (e.g., Cas12a-M28-1), Cas12a-M29 (e.g., Cas12a-M29-1), Cas12a-M30 (e.g., Cas12a-M30-1), Cas12a-M31 (e.g., Cas12a-M31-1), Cas12a-M32 (e.g., Cas12a-M32-1), Cas12a-M57, Cas12a-M58, Cas12a-M59, Cas12a-M60 (e.g., Cas12a-M60-9), Cas12a-M61, or Cas12a-M62), Cas12b, Cas12c, Cas12g, Cas12h, or Cas12i), Cas-Phi, CasX, CasY, C2c3, C2c2, C2c1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csx1, Csx15, Csf1, Csf2, Csf3, Csf4, and variants thereof. Numerous forms and variants of Cas nucleases are known in the art (e.g., spCas9, dCas9, nCas9, Cas9-SpRY, and Cas12a) and can have distinct characteristics, including for example recognition of distinct PAMs and PAM positions.

Other DNA binding nucleases can also be used in a prime editing enzyme. For example, prime editing systems can utilize zinc finger nucleases (ZFNs) (see, e.g., Urnov 2010 Nat Rev Genet. 11(9): 636-46) and transcription activator like effector nucleases (TALENs) (see, e.g., Joung 2013 Nat Rev Mol Cell Biol. 14(1): 49-55). For additional information regarding DNA-binding nucleases, see, e.g., US 2018/0312825.

In various embodiments, a prime editing system includes a prime editing gRNA (pegRNA) that specifies a target nucleic acid sequence and also specifies the sequence modification that the prime editing system introduces. The pegRNA includes a sequence complimentary to the target nucleic acid and recruits the prime editing enzyme to the target nucleic acid. A pegRNA includes, from 5′ to 3′: (a) a fragment that base pairs with a complementary target nucleic acid sequence (e.g., at least 80% identity between the fragment and the complement of the target nucleic acid, e.g., 80%, 85%, 90%, 91%, 92%, 93%, 94%, 95%, 96%, 97%, 98%, 99%, or 100% identity) (sometimes referred to as a “spacer”), where the fragment can be 10 to 40 nucleotides in length (e.g., equal to or about 10, 15, 16, 17, 18, 19, 20, 21, 22, 23, 24, 25, 30, 35 or 40 nucleotides in length, e.g., 17-24 or 17-20 nucleotides in length); (b) a sequence that forms a stemloop structure and binds with and/or recruits the catalytically impaired nuclease domain of a prime editing enzyme; (c) a fragment that includes a sequence that includes one or more modifications (e.g., one or more substitutions, insertions, and/or deletions) relative to the target nucleic acid sequence (sometimes referred to as a “template sequence”), and is complementary (excepting modifications) to the same target nucleic acid strand as (d); and (d) a fragment that includes a sequence complimentary to a target sequence (sometimes referred to as a “binding region” or “primer binding site” (PBS)), e.g., where the target sequence is upstream of an appropriate PAM site. In various embodiments, a PBS can be 5 to 20 nucleotides, e.g., 8 to 15 nucleotides in length. In various embodiments, a template sequence can be 10 to 20 nucleotides in length, or longer. Because pegRNAs include components characteristic of sgRNAs, they are sometimes described as extended sgRNAs. Any two fragments of a pegRNA can be, independently, associated directly or via a linker fragment.

A catalytically impaired nuclease domain of a prime editing enzyme can nick a target nucleic acid that includes an appropriate PAM to expose a 3′ flap and a 5′ flap. After nicking of the target nucleic acid, the released 3′ flap can hybridize to the PBS of the pegRNA, priming reverse transcription of the template fragment of the pegRNA that includes a modification of the target sequence, directly introducing the modification into the target nucleic acid to the 3′ flap. The product of reverse transcription, an edited 3′ flap that is “redundant” with the 5′ flap sequence produced by the nick (which includes the original, unedited sequence of the target nucleic acid), can then compete with the original and redundant 5′ flap sequence for reincorporation into the DNA duplex. Although the perfectly complimentary 5′ would likely be thermodynamically favored for hybridization to the non-edited strand, the 5′ flap is preferentially degraded by cellular endonucleases that are ubiquitous during lagging-strand DNA synthesis. After 5′ flap excision and ligation of the edited strand, permanent installation of the edit occurs through DNA repair of the non-edited that relies on the edited strand as a template. DNA repair of the non-edited strand can be promoted by contact with a secondary sgRNA that directs nicking of the non-edited strand. This additional nick stimulates re-synthesis of the non-edited strand using the edited strand as a template, resulting in a fully edited duplex. Prime editing systems can introduce any of one or more of the 12 types of point mutations (all possible nucleotide transitions and transversions), as well as insertions and/or deletions.

In various embodiments, a prime editing system is engineered to disrupt a PAM site of a target nucleic acid. Disruption of a PAM site of a target nucleic acid can reduce the probability of repeated editing of the particular target nucleic acid. In various embodiments, disruption of a PAM site in edited target nucleic acids can increase the efficiency of prime editing and/or gene therapy that includes prime editing.

Exemplary prime editing systems include PE1, PE2, and PE3. Each of these prime editing enzymes include a mutant Streptococcus pyogenes Cas9 nickase domain (H840A mutant) and a Moloney murine leukemia virus (M-MLV) reverse transcriptase (e.g., engineered to include D200N/T306K/W313F/T330P/L603W). PE1 includes a pegRNA and a prime editing enzyme that includes a Cas9 H840A nickase and wild type MLV RT. The Cas9 nickase acts only on the strand to be edited by the RT. PE2 includes pegRNA and a prime editing enzyme that includes a Cas9 H840A nickase and engineered MLV RT (D200N/T306K/W313F/T330P/L603W) demonstrated to improve editing efficiency. PE3 includes the same prime editing enzyme as PE2 (as well as a pegRNA) but further includes an sgRNA that targets the non-edited strand for nicking 14-116 nucleotides away from the site of the pegRNA-induced nick (PE3), where cellular mismatch repair pathways can fix the information introduced in the edited strand. Compared with PE2, the PE3b strategy demonstrate increased editing efficiency and lower levels of indel formation. A variant of the PE3 system called PE3b uses a nicking sgRNA that targets only the edited sequence, resulting in decreased levels of indel products by preventing nicking of the non-edited DNA strand until the other strand has been converted to the edited sequence.

Those of skill in the art will appreciate that a pegRNA or other targeting elements to generate a selected nucleic acid sequence modification in a target nucleic acid can be readily designed and implemented, e.g., based on available sequence information. Various tools for designing pegRNAs are available. For example, pegFinder is a web-based tool for pegRNA design (see, e.g., Chow 2020 Nat. Biomed. Eng. doi: 10.1038/s41551-020-00622-8). Another example of a web-based tool for pegRNA design is PrimeDesign (see, e.g., Hsu 2020 bioRxiv doi: 10.1101/2020.05.04.077750).

Prime editing systems do not require double-stranded DNA breaks. Prime editing systems provide precise control of the site at which the editing system modifies a target nucleic acid. Prime editing systems can be multiplexed to achieve editing of multiple targets using a single editing enzyme, optionally including therapeutic targets. The present disclosure includes that a prime editing system can include a plurality of pegRNAs (e.g., two or more, e.g., two, three, four, or five pegRNAs).

### I(C)(i)(b)(4). Zinc Finger Nucleases

The present disclosure includes Zinc Finger Nuclease. Zinc finger nucleases (ZFNs) are artificial restriction enzymes made by associating a sequence-targeted zinc-finger DNA-binding units with a nuclease domain (e.g., Fok1 nuclease domain) in a fusion protein. Each ZFN includes a nuclease domain (e.g., the cleavage domain of FokI) linked to an array of three to six zinc fingers zinc fingers (ZFs). For example, a ZFN can include several Cys₂His₂ ZFs in which each unit includes about 30 amino acids and specifically binds about 3 nucleotides. The ZFs provide a ZFN with the ability to bind a particular nucleic acid sequence. Because the FokI cleavage domain must dimerize to cut DNA, a monomer is not active, and cleavage does not occur at single binding sites. Thus, for example, ZFNs including three ZFs that together bind a 9-bp target function as ZFN dimers that specifically bind 18 bp of DNA per cleavage site. In some embodiments, ZFNs can include up to six ZFs per ZFN.

Cleave of a target nucleic acid by ZFNs induces cellular repair processes that can mediate modification of the nucleic acid. ZFN-induced double-strand breaks can lead to both targeted modification and targeted gene replacement. For example, if a ZFN-induced cleavage is resolved by non-homologous end joining, this can result in small deletions or insertions, which can lead to gene knockout. If a ZFN-induced cleavage is resolved by a homology-based process in the presence of a provided donor nucleic acid, small changes (e.g., one or a few nucleotides) or more (e.g., up to and including entire transgenes) can be introduced into the target nucleic acid.

### I(C)(i)(b)(5). TALENs for Modification of Nucleic Acids

The present disclosure includes Transcription Activator-Like Effector Nuclease (TALEN) editing systems. Various editing enzymes and systems can include a transcription activator-like (TAL) effector DNA binding domain and an endonuclease enzyme. An editing enzyme including a TAL effector DNA binding domain and an endonuclease can be referred to as a TALEN.

TAL effector DNA binding domains includes a plurality of monomers, each of which monomers binds one nucleotide in the target nucleic acid sequence. Each monomer includes 34 amino acids. In each monomer, positions 12 and 13 (referred to as the repeat variable diresidue, RVD) are highly variable and contribute to specific recognition of different nucleotides. The final monomer of a TAL effector DNA binding domain, which binds the nucleotide at the 3′-end of the recognition site, can be only 20 amino acids in length and therefore is sometimes referred to as a half-repeat. RVD sequences can be degenerate, as certain RVD combinations can bind to two or more nucleotides, e.g., with distinct efficiency. For example, RVDs include Asn and Ile (NI), Asn and Gly (NG), Asn and Asn (NN), and His and Asp (HD), which bind A, T, G, and C nucleotides, respectively.

In various embodiments, a TAL effector DNA binding domain is isolated from Xanthomonas spp. In various embodiments, a TALEN includes an endonuclease domain (e.g., a FokI domain), e.g., C-terminal to the TAL effector DNA binding domain.

TALENs work as pairs, the two members having target binding site on opposite DNA strands of the target nucleic acid sequence, with the targets separated by a small fragment (e.g., 12-25 bp) that can be referred to as a spacer sequence. Once a pair of TALENs have bound their target sites, the endonuclease (e.g., FokI) domains dimerize and cause a double-strand break in a spacer sequence. Non-homologous end joining (NHEJ) to resolve a DSB directly ligates DNA from either side of the double-strand break where there is very little or no sequence overlap for annealing. This repair mechanism can cause indels (insertion or deletion), or chromosomal rearrangement, which can disrupt genes at that target nucleic acid sequence. Alternatively, DNA can be introduced into a genome through NHEJ in the presence of exogenous double-stranded DNA fragments. Homology directed repair can also introduce foreign DNA at the DSB as the transfected double-stranded sequences are used as templates for the repair enzymes

### I(C)(i)(c). Small RNA Payload Expression Products

Small RNAs are short, non-coding RNA molecules that play a role in regulating gene expression. In particular embodiments, small RNAs are less than 200 nucleotides in length. In particular embodiments, small RNAs are less than 100 nucleotides in length. In particular embodiments, small RNAs are less than 50, 45, 40, 35, 30, 25, or 20 nucleotides in length. In particular embodiments, small RNAs are less than 20 nucleotides in length. In various embodiments, a small RNA has a length having a lower bound of 5, 10, 15, 20, 25, or 30 nucleotides and an upper bound of 20, 25, 30, 35, 40, 45, 50, 75, or 100 nucleotides. Small RNAs include but are not limited to microRNAs (miRNAs, Piwi-interacting RNAs (piRNAs), small interfering RNAs (siRNAs), small nucleolar RNAs (snoRNAs), tRNA-derived small RNAs (tsRNAs) small rDNA-derived RNAs (srRNAs), and small nuclear RNAs. Additional classes of small RNAs continue to be discovered.

In particular embodiments, interfering RNA molecules that are homologous to a target mRNA or to which the interfering RNA can hybridize can lead to degradation of the target mRNA molecule or reduced translation of the target mRNA, a process referred to as RNA interference (RNAi) (Carthew, Curr Opin. Cell. Biol. 13: 244-248, 2001). RNAi occurs in cells naturally to remove foreign RNAs (e.g., viral RNAs). In some instances, natural RNAi proceeds via fragments cleaved from free double-strand RNA (dsRNA) which direct the degradative mechanism to other similar RNA sequences. Alternatively, RNAi can be manufactured, for example, to silence the expression of target genes. Exemplary RNAi molecules include small hairpin RNA (shRNA, also referred to as short hairpin RNA) and small interfering RNA (siRNA).

Without limiting the disclosure, and without being bound by theory, RNA interference in nature and/or in some embodiments is typically a two-step process. In the first step, the initiation step, input dsRNA is digested into 21-23 nucleotide (nt) siRNA, probably by the action of Dicer, a member of the ribonuclease (RNase) III family of dsRNA-specific ribonucleases, which processes (cleaves) dsRNA (introduced directly or via a transgene or a virus) in an ATP-dependent manner. Successive cleavage events degrade the RNA to 19-21 base pair (bp) duplexes (siRNA), each with 2-nucleotide 3′ overhangs.

In a second step, an effector step, the siRNA duplexes bind to a nuclease complex to form the RNA-induced silencing complex (RISC). An ATP-dependent unwinding of the siRNA duplex is required for activation of the RISC. The active RISC then targets the homologous transcript by base pairing interactions and typically cleaves the mRNA into 12 nucleotide fragments from the 3′ terminus of the siRNA. Research indicates that each RISC contains a single siRNA and an RNase.

Because of the remarkable potency of RNAi, an amplification step within the RNAi pathway has been suggested. Amplification could occur by copying of the input dsRNAs which would generate more siRNAs, or by replication of the siRNAs formed. Alternatively or additionally, amplification could be effected by multiple turnover events of the RISC.

ShRNAs are single-stranded polynucleotides with a hairpin loop structure. The single-stranded polynucleotide has a loop segment linking the 3′ end of one strand in the double-stranded region and the 5′ end of the other strand in the double-stranded region. The double-stranded region is formed from a first sequence that is hybridizable to a target sequence, such as a polynucleotide encoding transgene, and a second sequence that is complementary to the first sequence, thus the first and second sequence form a double stranded region to which the linking sequence connects the ends of to form the hairpin loop structure. The first sequence can be hybridizable to any portion of a polynucleotide encoding transgene. The double-stranded stem domain of the shRNA can include a restriction endonuclease site.

Transcription of shRNAs is initiated at a polymerase III (Pol III) promoter and is thought to be terminated at position 2 of a 4-5-thymine transcription termination site. Upon expression, shRNAs are thought to fold into a stem-loop structure with 3′ UU-overhangs; subsequently, the ends of these shRNAs are processed, converting the shRNAs into siRNA-like molecules of 21-23 nucleotides.

The stem-loop structure of shRNAs can have optional nucleotide overhangs, such as 2-bp overhangs, for example, 3′ UU overhangs. While there may be variation, stems typically range from 15 to 49, 15 to 35, 19 to 35, 21 to 31 bp, or 21 to 29 bp, and the loops can range from 4 to 30 bp, for example, 4 to 23 bp. In particular embodiments, shRNA sequences include 45-65 bp; 50-60 bp; or 51, 52, 53, 54, 55, 56, 57, 58, or 59 bp. In particular embodiments, shRNA sequences include 52 or 55 bp. In particular embodiments, siRNAs have 15-25 bp. In particular embodiments, siRNAs have 16, 17, 18, 19, 20, 21, 22, 23, or 24 bp. In particular embodiments, siRNAs have 19 bp. The skilled artisan will appreciate, however, that siRNAs having a length of less than 16 nucleotides or greater than 24 nucleotides can also function to mediate RNAi. Longer RNAi agents have been demonstrated to elicit an interferon or Protein kinase R (PKR) response in certain mammalian cells which may be undesirable. Preferably the RNAi agents do not elicit a PKR response (i.e., are of a sufficiently short length). However, longer RNAi agents may be useful, for example, in situations where the PKR response has been downregulated or dampened by alternative means.

In certain illustrative embodiments, the present disclosure includes an adenoviral vector payload that encodes an shRNA targeted to the gene encoding BCL11A, where the shRNA causes decreased translation of BCL11A.

### I(C)(ii). Payload Regulatory Sequences

Promoters can include general promoters, tissue-specific promoters, cell-specific promoters, and/or promoters specific for the cytoplasm. Promoters may include strong promoters, weak promoters, constitutive expression promoters, and/or inducible (conditional) promoters. Inducible promoters direct or control expression in response to certain conditions, signals, or cellular events. For example, a promoter can be an inducible promoter that requires a particular ligand, small molecule, transcription factor, hormone, or hormone protein in order to effect transcription from the promoter

In various embodiments, a promoter sequence can be a native promoter sequence. A native promoter sequence, or minimal promoter sequence, can refer to a sequence derived from a single contiguous sequence positioned 5′ of a coding sequence in a reference genome. A native promoter sequence can include a core promoter and an associated 5′UTR. In particular embodiments, a 5′UTR can include an intron. In various embodiments, a promoter sequence can be a composite promoter sequence. In various embodiments, a composite promoter sequence can refer to a promoter sequence that includes portions derived from at least two distinct sources, e.g., from two non-contiguous portions of a reference genome, from two distinct genomes, or from any two distinct source sequences. For example, in certain embodiments, a composite promoter sequence includes a sequence derived from a single contiguous sequence positioned 5′ of a coding sequence in a reference genome and a sequence derived from another portion of the reference genome, e.g., an enhancer (e.g., a distal enhancer).

In particular embodiments, a promoter can be a wild type promoter sequence or a sequence with one or more changes relative to a reference promoter (e.g., one or more insertions, point mutations, or deletions). In particular embodiments, a promoter sequence differs from a wild type or other reference promoter sequence by having 1 change per 20 nucleotide stretch, 2 changes per 20 nucleotide stretch, 3 changes per 20 nucleotide stretch, 4 changes per 20 nucleotide stretch, or 5 changes per 20 nucleotide stretch. In particular embodiments, a promoter sequence can differ from a wild type or reference sequence by 1, 2, 3, 4, 5, 6, 7, 8, 9, or 10 nucleotide differences. A promoter can have a length of, e.g., 50 to 3,000 or more nucleotides, e.g., 100-1,000, 100-2,000, 100-3,000, 500-1,000, 500-2,000, 500-3,000, 1,000-2,000, or 1,000-3,000 nucleotides.

In various embodiments, a promoter is non-specific in that it causes expression of an operably linked coding sequence in cells or tissues of diverse types. In various embodiments, a promoter is a ubiquitous promoter. In various embodiments, a ubiquitous promoter can be selected from, e.g., a CMV promoter, RSV promoter, or SV40 promoter.

Coding sequences of the present disclosure can additionally be associated with sequences that enhance the stability of mRNA transcripts, such as an insulator and/or a polyA tail.

### I(C)(iii). Selection Sequences

In particular embodiments, vectors include a selection element including a selection cassette. In particular embodiments, a selection cassette includes a promoter, a cDNA that adds or confers resistance to a selection agent, and a poly A sequence that enables stopping the transcription of this independent transcriptional element.

A selection cassette can encode one or more proteins that (a) confer resistance to antibiotics or other toxins, (b) complement auxotrophic deficiencies, or (c) supply critical nutrients not available from complex media, e.g., the gene encoding D-alanine racemase for Bacilli. Any number of selection systems may be used to recover transformed cell lines. In particular embodiments, a positive selection cassette includes resistance genes to neomycin, hygromycin, ampicillin, puromycin, phleomycin, zeomycin, blasticidin, or viomycin. In particular embodiments, a positive selection cassette includes the DHFR (dihydrofolate reductase) gene providing resistance to methotrexate, the MGMT^{P140K} gene responsible for the resistance to O⁶BG/BCNU, the HPRT (Hypoxanthine phosphoribosyl transferase) gene responsible for the transformation of specific bases present in the HAT selection medium (aminopterin, hypoxanthine, thymidine), and other genes for detoxification with respect to some drugs. In particular embodiments, the selection agent includes neomycin, hygromycin, puromycin, phleomycin, zeomycin, blasticidin, viomycin, ampicillin, O⁶BG/BCNU, methotrexate, tetracycline, aminopterin, hypoxanthine, thymidine kinase, DHFR, Gln synthetase, or ADA.

In particular embodiments, a negative selection cassette includes a gene encoding an expression product that transforms a substrate present in (e.g., delivered to) a subject or system (e.g., a culture medium) into a toxic substance, thereby sensitizing cells that expresses the gene. In various embodiments, for example, a payload is engineered such that proper integration into a target genome disrupts expression of the negative selection gene. A negative selection cassette can include a gene encoding diphtheria toxin A-fragment (DTA) (Yagi et al., Anal Biochem. 214(1): 77-86, 1993; Yanagawa et al., Transgenic Res. 8(3): 215-221, 1999) or a thymidine kinase gene of the Herpes virus (HSV TK) sensitive to the presence of ganciclovir or FIAU. In various embodiments, a negative selection cassette includes an HPRT gene for negative selection in the presence of 6-thioguanine (6TG).

In particular embodiments, a selection cassette includes MGMT^{P140K} as described in Olszko et al. (Gene Therapy 22: 591-595, 2015). In particular elements, the selection agent includes O⁶BG/BCNU.

The MGMT gene encodes human alkyl guanine transferase (hAGT), a DNA repair protein that confers resistance to the cytotoxic effects of alkylating agents, such as nitrosoureas and temozolomide (TMZ). 6-benzylguanine (6-BG) is an inhibitor of AGT that potentiates nitrosourea toxicity and is co-administered with TMZ to potentiate the cytotoxic effects of this agent. Several mutant forms of MGMT that encode variants of AGT are highly resistant to inactivation by 6-BG but retain their ability to repair DNA damage (Maze et al., J. Pharmacol. Exp. Ther 290: 1467-1474, 1999). MGMT^{P140K}-based drug resistant gene therapy has been shown to confer chemoprotection to mouse, canine, rhesus macaques, and human cells, specifically hematopoietic cells (Zielske et al., J. Clin. Invest. 112: 1561-1570, 2003; Pollok et al., Hum. Gene Ther 14: 1703-1714, 2003; Gerull et al., Hum. Gene Ther. 18: 451-456, 2007; Neff et al., Blood 105: 997-1002, 2005; Larochelle et al., J. Clin. Invest. 119: 1952-1963, 2009; Sawai et al., Mol. Ther. 3: 78-87, 2001).

In particular embodiments, combination with an in vivo selection cassette will be a critical component for diseases without a selective advantage of gene-corrected cells. For example, in SCID and some other immunodeficiencies and FA, corrected cells have an advantage and only transducing the therapeutic gene into a “few” HSPCs is sufficient for therapeutic efficacy. For other diseases like hemoglobinopathies (i.e., sickle cell disease and thalassemia) in which therapeutically modified cells do not demonstrate a competitive advantage, in vivo selection of the modified cells, e.g., for expression of an in vivo selection cassette such as MGMT^{P140K}, will select for the few transduced HSPCs, allowing an increase in the gene corrected cells and in order to achieve therapeutic efficacy. This approach can also be applied to HIV by making HSPCs resistant to HIV in vivo rather than ex vivo genetic modification.

### I(C)(iv). Stuffer Sequences

In particular embodiments, the vector includes a stuffer sequence. In particular embodiments, the stuffer sequence may be added to render the genome at a size near that of wild-type length. Stuffer is a term generally recognized in the art intended to define functionally inert sequence intended to extend the length of the genome.

The stuffer sequence is used to achieve efficient packaging and stability of the vector. In particular embodiments, the stuffer sequence is used to render the genome size between 70% and 110% of that of the wild type virus.

The stuffer sequences can be any DNA, preferably of mammalian origin. In a preferred embodiment of the invention, stuffer sequences are non-coding sequences of mammalian origin, for example intronic fragments.

The stuffer sequence, when used to keep the size of the vector a predetermined size, can be any non-coding sequence or sequence that allows the genome to remain stable in dividing or nondividing cells. These sequences can be derived from other viral genomes (e.g. Epstein bar virus) or organism (e.g. yeast). For example, these sequences could be a functional part of centromeres and/or telomeres.

### I(C)(v). Payload Integration and Support Vectors

Gene therapy often requires integration of a desired nucleic acid payload into the genome of a target cell. A variety of systems can be designed and/or used for integration of a payload into a host or target cell genome. Various such systems can include one or more of certain payload sequence features and support vectors and support genomes (support genomes).

One means of engineering adenoviral vectors that integrate a payload into a host cell genome has been to produce integrating viral hybrid vectors. Integrating viral hybrid vectors combine genetic elements of a vector that efficiently transduces target cells with genetic elements of a vector that stably integrates its vector payload. Integration elements of interest, e.g., for use in combination with adenoviral vectors, have included those of bacteriophage integrase PHiC31, retrotransposons, retrovirus (e.g., LTR-mediated or retrovirus integrate-mediated), zinc-finger nuclease, DNA-binding domain-retroviral integrase fusion proteins, AAV (e.g., AAV-ITR or AAV-Rep protein-mediated), and Sleeping Beauty (SB) transposase.

Ad3, 5, 7, 11, 14, 16, 21, 34, 35, 37, or 50 vectors described herein can optionally include transposable elements including transposases and transposons. Transposases can include integrases from retrotransposons or of retroviral origin, as well as an enzyme that is a component of a functional nucleic acid-protein complex capable of transposition and which is mediating transposition. A transposition reaction includes a transposon and a transposase or an integrase enzyme. In particular embodiments, the efficiency of integration, the size of the DNA sequence that can be integrated, and the number of copies of a DNA sequence that can be integrated into a genome can be improved by using such transposable elements. Transposons include a short nucleic acid sequence with terminal repeat sequences upstream and downstream of a larger segment of DNA. Transposases bind the terminal repeat sequences and catalyze the movement of the transposon to another portion of the genome.

A number of transposases have been described in the art that facilitate insertion of nucleic acids into the genome of vertebrates, including humans. Examples of such transposases include sleeping beauty (“SB”, e.g., derived from the genome of salmonid fish); piggyback (e.g., derived from lepidopteran cells and/or the Myotis lucifugus); mariner (e.g., derived from Drosophila); frog prince (e.g., derived from Rana pipiens); Tol1; Tol2 (e.g., derived from medaka fish); TcBuster (e.g., derived from the red flour beetle Tribolium castaneum), Helraiser, Himar1, Passport, Minos, Ac/Ds, PIF, Harbinger, Harbinger3-DR, HSmar1, and spinON.

The PiggyBac (PB) transposase is a compact functional transposase protein that is described in, for example, Fraser et al., Insect Mol. Biol., 1996, 5, 141-51; Mitra et al., EMBO J., 2008, 27, 1097-1109; Ding et al., Cell, 2005, 122, 473-83; and U.S. Pat. Nos. 6,218,185; 6,551,825; 6,962,810; 7,105,343; and 7,932,088. Hyperactive piggyBac transposases are described in U.S. Pat. No. 10,131,885.

Additional information on DNA transposons can be found, for instance, in Muñoz-López & García Pérez, Curr Genomics, 11(2):115-128, 2010.

Sleeping Beauty is described in Ivics et al. Cell 91, 501-510, 1997; Izsvak et al., J. Mol. Biol., 302(1):93-102, 2000; Geurts et al., Molecular Therapy, 8(1): 108-117, 2003; Mates et al. Nature Genetics 41:753-761, 2009; and U.S. Pat. Nos. 6,489,458; 7,148,203; and 7,160,682; US Publication Nos. 2011/117072; 2004/077572; and 2006/252140. In certain embodiments, the Sleeping Beauty transposase enzyme is a Hyperactive Sleeping Beauty SB100x transposase enzyme. SB transposons are most efficiently transposed when present in circularized nucleic acid molecules (Yant et al., Nature Biotechnology, 20: 999-1005, 2002).

Systematic mutagenesis studies have been undertaken to increase the activity of the SB transposase. For example, Yant et al. undertook the systematic exchange of the N-terminal 95 AA of the SB transposase for alanine (Mol. Cell Biol. 24: 9239-9247, 2004). Ten of these substitutions caused hyperactivity between 200-400% as compared to SB10 as a reference. SB16, described in Baus et al. (Mol. Therapy 12: 1148-1156, 2005) was reported to have a 16-fold activity increase as compared to SB10. Additional hyperactive SB variants are described in Zayed et al. (Molecular Therapy 9(2):292-304, 2004) and U.S. Pat. No. 9,840,696.

SB transposases transpose nucleic acid transposon payloads that are positioned between SB ITRs. Various SB ITRs are known in the art. In some embodiments, an SB ITR is a 230 bp sequence including imperfect direct repeats of 32 bp in length that serve as recognition signals for the transposase.

In various embodiments, an Ad3, 5, 7, 11, 14, 16, 21, 34, 35, 37, or 50 donor vector or genome includes a payload that includes SB100x transposon inverted repeats that flank an integration element that includes at least one coding sequence that encodes a β-globin expression product or a γ-globin expression product.

In various embodiments, an adenoviral transposition system includes an Ad3, 5, 7, 11, 14, 16, 21, 34, 35, 37, or 50 donor vector or genome that includes an integration element flanked by transposon inverted repeats, and can further include an adenoviral support vector or support genome. In various embodiments, a support vector includes (i) the adenoviral capsid; and (ii) an adenoviral support genome including a nucleic acid sequence encoding a transposase that corresponds to the inverted repeats that flank the integration element. Accordingly, in various embodiments, at least one function of a support vector or support genome can be to encode, express, and/or deliver to a target cell a transposase for transposition of an integration element present in a donor vector administered to the target cell. For instance, in some embodiments, an Ad3, 5, 7, 11, 14, 16, 21, 34, 35, 37, or 50 donor vector or genome includes SB100x transposon inverted repeats that flank an integration element that includes at least one coding sequence that encodes a β-globin expression product or a γ-globin expression product, and a support vector or support genome includes a coding sequence that encodes SB100x transposase. In certain embodiments, an integration element is flanked by recombinase direct repeats, e.g., where the integration element is flanked by transposon inverted repeats and the transposon inverted repeats are flanked by recombinase direct repeats. In certain such embodiments, at least one function of a support vector or support genome can be to encode, express, and/or deliver to a target cell a recombinase for recombination of recombinase sites present in a donor vector administered to the target cell. In various embodiments, a support vector or support genome can encode, express, and/or deliver to a target cell a recombinase for recombination of recombinase sites present in a donor vector administered to the target cell and also encode, express, and/or deliver to a target cell a transposase for transposition of an integration element present in a donor vector administered to the target cell.

Particular embodiments disclosed herein also use site-specific recombinase systems. In these embodiments, in addition to at least one therapeutic gene, the transposon including transposase-recognized inverted repeats also includes at least one recombinase-recognized site. Thus, in particular embodiments, The present disclosure also provides methods of integrating a therapeutic gene into the genome including administering: (a) a transposon including the therapeutic gene, where the therapeutic gene is flanked by (i) an inverted repeat sequence recognized by a transposase and (ii) a recombinase-recognized site; and b) a transposase and recombinase that serve to excise the therapeutic gene from a plasmid, episome, or transgene and integrate the therapeutic gene into the genome. In some embodiments, the protein(s) of (b) are administered as a nucleic acid encoding the protein(s). In some embodiments, the transposon and the nucleic acids encoding the protein(s) of (b) are present on separate vectors. In some embodiments, the transposon and nucleic acid encoding the protein(s) of (b) are present on the same vector. When present on the same vector, the portion of the vector encoding the protein(s) of (b) are located outside the portion carrying the transposon of (a). In other words, the transposase and/or recombinase encoding region is located external to the region flanked by the inverted repeats and/or recombinase-recognition site. In the aforementioned methods, the transposase protein recognizes the inverted repeats that flank an inserted nucleic acid, such as a nucleic acid that is to be inserted into a target cell genome. The use of recombinases and recombinase-recognized sites can increase the size of a transposon that can be integrated into a genome further.

Examples of recombinase systems include the Flp/Frt system, the Cre/loxP system, the Dre/rox system, the Vika/vox system, and the PhiC31 system. The Flp/Frt DNA recombinase system was isolated from Saccharomyces cerevisiae. The Flp/Frt system includes the recombinase Flp (flippase) that catalyzes DNA-recombination on its Frt recognition sites. Variants of the Flp protein include GenBank accession no. ABD57356.1 and GenBank accession no. ANW61888.1.

The Cre/loxP system is described in, for example, EP 02200009B1. Cre is a site-specific DNA recombinase isolated from bacteriophage P1. The recognition site of the Cre protein is a nucleotide sequence of 34 base pairs, the loxP site. Cre recombines the 34 bp loxP DNA sequence by binding to the 13 base pair inverted repeats and catalyzing strand cleavage and re-ligation within the spacer region. The staggered DNA cuts made by Cre in the spacer region are separated by 6 base pairs to give an overlap region that acts as a homology sensor to ensure that only recombination sites having the same overlap region recombine. Variants of the lox recognition site that can also be used include: lox2272; lox511; lox66; lox71; loxM2; and lox5171. The VCre/VloxP recombinase system was isolated from Vibrio plasmid p0908. The sCre/SloxP system is described in WO 2010/143606. The Dre/rox system is described in U.S. Pat. Nos. 7,422,889 and 7,915,037B2. It generally includes a Dre recombinase isolated from Enterobacteria phage D6 and the rox recognition site. The Vika/vox system is described in U.S. Pat. No. 10,253,332. Additionally, the PhiC31 recombinase recognizes the AttB/AttP binding sites.

The amount of vector nucleic acid including the transposon (including inverted repeats and/or recombinase recognition sites), and in various embodiments the amount of vector nucleic acid encoding the transposase and/or recombinase, introduced into the cell is/are sufficient to provide for the desired excision and insertion of the transposon nucleic acid into the target cell genome. As such, the amount of vector nucleic acid introduced should provide for a sufficient amount of transposase activity and/or recombinase activity and a sufficient copy number of the transposon that is desired to be inserted into the target cell genome. Particular embodiments include a 1:1; 1:2; or 1:3 ratio of transposon to transposase/recombinase.

The subject methods result in stable integration of the nucleic acid into the target cell genome. By stable integration is meant that the nucleic acid remains present in the target cell genome for more than a transient period of time and passes on a part of the chromosomal genetic material to the progeny of the target cell.

As indicated previously, particular embodiments utilize homology arms to facilitate targeted insertion of genetic constructs utilizing homology directed repair. Homology arms can be any length with sufficient homology to a genomic sequence at a cleavage site, e.g. 70%, 80%, 85%, 90%, 95%, or 100% homology with the nucleotide sequences flanking the cleavage site, e.g., within 50 bases or less of the cleavage site, e.g., within 30 bases, within 15 bases, within 10 bases, within 5 bases, or immediately flanking the cleavage site, to support HDR between it and the genomic sequence to which it bears homology. Homology arms are generally identical to the genomic sequence, for example, to the genomic region in which the double stranded break (DSB) occurs. However, as indicated, absolute identity is not required.

Particular embodiments can utilize homology arms with 25, 50, 100, or 200 nucleotides (nt), or more than 200 nt of sequence homology between a homology-directed repair template and a targeted genomic sequence (or any integral value between 10 and 200 nucleotides, or more). In particular embodiments, homology arms are 40-1000 nt in length. In particular embodiments, homology arms are 500-2500 base pairs, 700-2000 base pairs, or 800-1800 base pairs. In particular embodiments, homology arms include at least 800 base pairs or at least 850 base pairs. The length of homology arms can also be symmetric or asymmetric.

Particular embodiment can utilize first and/or second homology arms each including at least 25, 50, 100, 200, 400, 600, 800, 1,000, 1,200, 1,400, 1,600, 1,800, 2,000, 2,500, or 3,000 nucleotides or more, having sequence identity or homology with a corresponding fragment of a target genome. In some embodiments, first and/or second homology arms each include a number of nucleotides having sequence identity or homology with a corresponding fragment of a target genome that has a lower bound of 25, 50, 100, 200, 400, 600, 800, 1,000, 1,200, 1,400, 1,600, or 1,800 nucleotides and an upper bound of 1,000, 1,200, 1,400, 1,600, 1,800, 2,000, 2,500, or 3,000 nucleotides. In some embodiments, first and/or second homology arms each include a number of nucleotides having sequence identity or homology with a corresponding fragment of a target genome that is between 40 and 1,000 nucleotides, between 500 and 2,500 nucleotides, between 700 and 2,000 nucleotides, or between 800 and 1800 nucleotides, or that has a length of at least 800 nucleotides or at least 850 nucleotides. First and second homology arms can have same, similar, or different lengths.

For additional information regarding homology arms, see Richardson et al., Nat Biotechnol. 34(3):339-44, 2016.

In particular embodiments, genetic constructs (e.g., genes leading to expression of a therapeutic product within a cell) are precisely inserted within genomic safe harbors. Genomic safe harbor sites are intragenic or extragenic regions of the genome that are able to accommodate the predictable expression of newly integrated DNA without adverse effects on the host cell. A useful safe harbor must permit sufficient transgene expression to yield desired levels of the encoded protein. A genomic safe harbor site also must not alter cellular functions. Methods for identifying genomic safe harbor sites are described in Sadelain et al., Nature Reviews 12:51-58, 2012; and Papapetrou et al., Nat Biotechnol. 29(1):73-8, 2011. In particular embodiments, a genomic safe harbor site meets one or more (one, two, three, four, or five) of the following criteria: (i) distance of at least 50 kb from the 5′ end of any gene, (ii) distance of at least 300 kb from any cancer-related gene, (iii) within an open/accessible chromatin structure (measured by DNA cleavage with natural or engineered nucleases), (iv) location outside a gene transcription unit and (v) location outside ultraconserved regions (UCRs), microRNA or long non-coding RNA of the genome.

In particular embodiments, to meet the criteria of a genomic safe harbor, chromatin sites must be >150 kb away from a known oncogene, >30 kb away from a known transcription start site; and have no overlap with coding mRNA. In particular embodiments, to meet the criteria of a genomic safe harbor, chromatin sites must be >200 kb away from a known oncogene, >40 kb away from a known transcription start site; and have no overlap with coding mRNA. In particular embodiments, to meet the criteria of a genomic safe harbor, chromatin sites must be >300 kb away from a known oncogene, >50 kb away from a known transcription start site; and have no overlap with coding mRNA. In particular embodiments, a genomic safe harbor meets the preceding criteria (>150 kb, >200 kb or >300 kb away from a known transcription start site; and have no overlap with coding mRNA >40 kb, or >50 kb away from a known transcription start site with no overlap with coding mRNA) and additionally is 100% homologous between an animal of a relevant animal model and the human genome to permit rapid clinical translation of relevant findings.

In particular embodiments, a genomic safe harbor meets criteria described herein and also demonstrates a 1:1 ratio of forward:reverse orientations of lentiviral integration further demonstrating the locus does not impact surrounding genetic material.

Particular genomic safe harbors sites include CCR5, HPRT, AAVS1, Rosa and albumin. See also, e.g., U.S. Pat. Nos. 7,951,925 and 8,110,379; U.S. Publication Nos. 2008/0159996; 2010/00218264; 2012/0017290; 2011/0265198; 2013/0137104; 2013/0122591; 2013/0177983 and 2013/0177960 for additional information and options for appropriate genomic safe harbor integration sites.

Various technologies known in the art can be used to direct integration of an integration element at specific genomic loci such as genomic safe harbors. For example AAV-mediated gene targeting, as well as homologous recombination enhanced by the introduction of DNA double-strand breaks using site-specific endonucleases (zinc-finger nucleases, meganucleases, transcription activator-like effector (TALE) nucleases), and CRISPR/Cas systems are all tools that can mediate targeted insertion of foreign DNA at predetermined genomic loci such as genomic safe harbors.

In certain embodiments, integration of an integration element at specific genomic loci such as genomic safe harbors can include homology-directed integration using CRISPR enzyme-mediated cleavage of a target genome. CRISPR enzyme (e.g., Cas9) cleaves double stranded DNA at a site specified by a guide RNA (gRNA). The double strand break can be repaired by homology-directed repair (HDR) when a donor template (such as an Ad3, 5, 7, 11, 14, 16, 21, 34, 35, 37, or 50 payload integration element including left and right homology arms) is present. In various such methods, an integration element is a “repair template” in that it includes left and right homology arms (e.g., of 500-3,000 bp) for insertion into a cleaved target genome. CRISPR-mediated gene insertion can be several orders of magnitude more efficient compared with spontaneous recombination of DNA template, demonstrating that CRISPR-mediated gene insertion can be an effective tool for genome editing. Exemplary methods of homology-directed integration of a nucleic acid sequence into a specified genomic locus are known in the art, e.g., in Richardson et al. (Nat Biotechnol. 34(3):339-44, 2016).

### II. Target Cell Populations

In various embodiments, donor vectors and genomes of the present disclosure can selectively target (e.g., selectively enter and/or selectively transduce) one or more hematopoietic cell types disclosed herein. Selective targeting includes, without limitation, preferential targeting (e.g., binding, entry, transduction, and/or modification) of one or more cell types as compared to one or more reference cell types. In various embodiments, the one or more preferentially targeted cell types are, or include one or more of, hematopoietic cell types disclosed herein. In various embodiments, the one or more reference cell types are, or include one or more of, hematopoietic cell types disclosed herein. In various embodiments, none of the reference cell types are the same as any of the preferentially targeted cell types. Accordingly, reference to a vector selectively targeting a hematopoietic cell type can, but does not necessarily, mean or imply, that the vector does not also target (e.g., selectively target) one or more other hematopoietic cell types. In various embodiments, preferential targeting refers specifically to the comparison of one single hematopoietic cell type to a reference group including two or more hematopoietic cell types. In various embodiments, preferential targeting refers specifically to the comparison of a group including two or more hematopoietic cell types to a single reference hematopoietic cell type. In various embodiments, preferential targeting refers specifically to the comparison of a group including two or more hematopoietic cell types to a reference group including two or more hematopoietic cell types. In various embodiments, a hematopoietic cell type is a stem cell type, a progenitor cell type, or a further differentiated cell type (e.g., a terminally differentiated cell type). In various embodiments, a group of hematopoietic cell types can be stem cells, progenitor cells, or cells of a particular lineage, e.g., a lineage identified by the least differentiated member of the identified group of cells and including one or more or all more differentiated hematopoietic cells derived therefrom. Selective targeting includes but does not require that preferentially targeted hematopoietic cell type(s) are preferentially targeted as compared to all other hematopoietic cell types. In various embodiments, selective targeting includes infection and/or transduction of at least 0.1%, at least 0.2%, at least 0.3%, at least 0.4%, at least 0.5%, at least 0.6%, at least 0.7%, at least 0.8%, at least 0.9%, at least 1%, at least 5%, at least 10%, at least 15%, at least 20%, at least 25% at least 30%, at least 35%, at least 40%, at least 45%, at least 50%, at least 55%, at least 60%, at least 65%, at least 70%, at least 75%, at least 80%, at least 85%, at least 90%, or at least 95% of the cells in a population of cells of the preferentially targeted hematopoietic cell type.

Hematopoietic cell types (e.g., target hematopoietic cell types) of the present disclosure include hematopoietic cells of all lineages and stages of hematopoietic cell differentiation. Target cell types of the present disclosure include, without limitation, HSCs (e.g., CD34⁺ long-term (LT)-HSCs and/or CD34⁺ short-erm (ST)-HSCs), common lymphoid progenitors (CLPs), T cells, NK cells, colony forming unit (CFU)-pre B cells, B cells, common myeloid progenitors (CMPs), granulocyte-macrophage progenitors (GMPs), CFU-M cells, monoblasts, monocytes, macrophages, CFU-G cells, myeloblasts, granulocytes, neutrophils, eosinophils, basophils, megakaryocyte-erythrocyte progenitors (MEPs), BFU-E cells, CFU-E cells, erythroblasts, erythrocytes, CFU-Mk cells, megakaryocytes, and/or platelets. Hematopoietic cell types (e.g., target hematopoietic cell types) of the present disclosure include CD34⁺ hematopoietic cells.

HSCs can be targeted for in vivo genetic modification by binding CD46. HSCs or subsets thereof can also be identified by any of the following marker profiles: CD34+; Lin−/CD34+/CD38−/CD45RA−/CD90+/CD49f+ (HSC1); CD34+/CD38−/CD45RA−/CD90−/CD49f+/(HSC2). In various embodiments, human HSC1 can be identified by any of the following profiles: CD34+/CD38−/CD45RA−/CD90+ or CD34+/CD45RA−/CD90+ and mouse LT-HSC can be identified by Lin-Sca1+ckit+CD150+CD48−Flt3−CD34− (where Lin represents the absence of expression of any marker of mature cells including CD3, CD4, CD8, CD11b, CD11c, NK1.1, Gr1, and TER119). In particular embodiments, HSC are identified by a CD164+ profile. In particular embodiments, HSC are identified by a CD34+/CD164+ profile. For additional information regarding HSC marker profiles, see WO2017/218948.

Hematopoietic cells can be beneficially caused to encode and/or express various payloads provided herein, including without limitation TCRs and CARs (see, e.g., Gschweng et al. Immunol Rev. 2014 January; 257(1): 237-249).

Hematopoietic cell types that can be targeted by vectors of the present disclosure include T cells. Several different subsets of T-cells have been discovered, each with a distinct function. For example, a majority of T-cells have a T-cell receptor (TCR) existing as a complex of several proteins. The actual T-cell receptor is composed of two separate peptide chains, which are produced from the independent T-cell receptor alpha and beta (TCRα and TCRβ) genes and are called α- and β-TCR chains.

γδ T-cells represent a small subset of T-cells that possess a distinct T-cell receptor (TCR) on their surface. In γδ T-cells, the TCR is made up of one γ-chain and one δ-chain. This group of T-cells is much less common (2% of total T-cells) than the αβ T-cells.

CD3 is expressed on all mature T cells. Activated T-cells express 4-1BB (CD137), CD69, and CD25. CD5 and transferrin receptor are also expressed on T-cells.

T-cells can further be classified into helper cells (CD4+ T-cells) and cytotoxic T-cells (CTLs, CD8+ T-cells), which include cytolytic T-cells. T helper cells assist other white blood cells in immunologic processes, including maturation of B cells into plasma cells and activation of cytotoxic T-cells and macrophages, among other functions. These cells are also known as CD4+ T-cells because they express the CD4 protein on their surface. Helper T-cells become activated when they are presented with peptide antigens by MHC class II molecules that are expressed on the surface of antigen presenting cells (APCs). Once activated, they divide rapidly and secrete small proteins called cytokines that regulate or assist in the active immune response.

Cytotoxic T-cells destroy virally infected cells and tumor cells, and are also implicated in transplant rejection. These cells are also known as CD8+ T-cells because they express the CD8 glycoprotein on their surface. These cells recognize their targets by binding to antigen associated with MHC class I, which is present on the surface of nearly every cell of the body.

In particular embodiments, CARs are genetically modified to be expressed in cytotoxic T-cells.

“Central memory” T-cells (or “TCM”) as used herein refers to an antigen experienced CTL that expresses CD62L or CCR7 and CD45RO on the surface thereof, and does not express or has decreased expression of CD45RA as compared to naive cells. In particular embodiments, central memory cells are positive for expression of CD62L, CCR7, CD25, CD127, CD45RO, and CD95, and have decreased expression of CD45RA as compared to naive cells.

“Effector memory” T-cell (or “TEM”) as used herein refers to an antigen experienced T-cell that does not express or has decreased expression of CD62L on the surface thereof as compared to central memory cells and does not express or has decreased expression of CD45RA as compared to a naive cell. In particular embodiments, effector memory cells are negative for expression of CD62L and CCR7, compared to naive cells or central memory cells, and have variable expression of CD28 and CD45RA. Effector T-cells are positive for granzyme B and perforin as compared to memory or naive T-cells.

“Naive” T-cells as used herein refers to a non-antigen experienced T cell that expresses CD62L and CD45RA and does not express CD45RO as compared to central or effector memory cells. In particular embodiments, naive CD8+ T lymphocytes are characterized by the expression of phenotypic markers of naive T-cells including CD62L, CCR7, CD28, CD127, and CD45RA.

Hematopoietic cell types that can be targeted by vectors of the present disclosure include B cells. B cells are mediators of the humoral response and are responsible for production and release of antibodies specific to an antigen. Several types of B cells exist which can be characterized by key markers. In general, immature B cells express CD19, CD20, CD34, CD38, and CD45R, and as they mature the key expressed markers are CD19 and IgM.

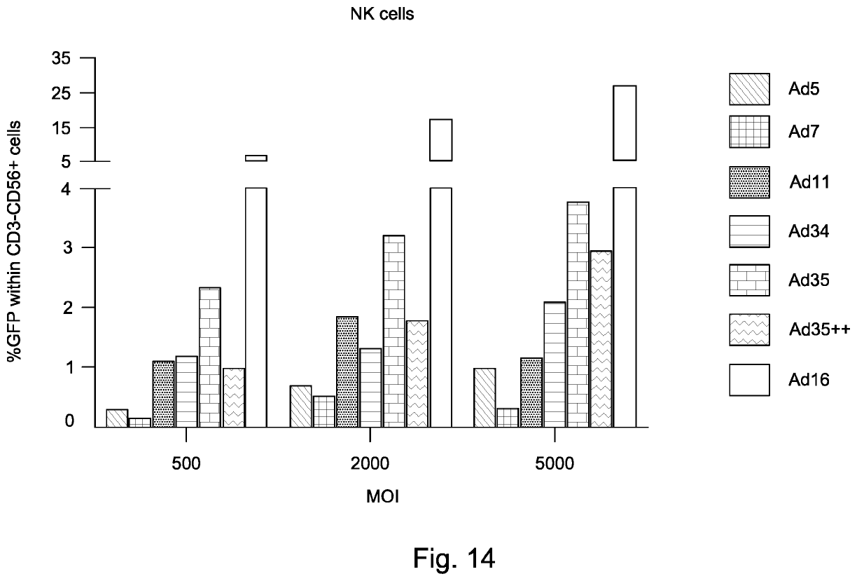

For avoidance of doubt, in various embodiments, vectors and genomes of the present disclosure can infect and/or transduce, and/or selectively target, CD11+/CD14+ monocytes, CD3+ T cells, CD3−/CD56+ NK cells, and/or CD20+ B cells. In various embodiments, CD11+/CD14+ monocytes and/or a CD11+/CD14+ phenotype can refer to cells found to express CD11 and CD14, e.g., based on binding of cells with a labelled anti-CD11 antibody and a labelled anti-CD14 antibody, e.g., as set forth in Example 10 and/or FIG. 14. In various embodiments, CD3+ T cells and/or a CD3+ phenotype can refer to cells found to express CD3, e.g., based on binding of cells with a labelled anti-CD3 antibody, e.g., as set forth in Example 10 and/or FIG. 14. In various embodiments, CD3−/CD56+NK cells and/or a CD3-/CD56+ phenotype can refer to cells found to express CD56 and not express CD3, e.g., based on binding of cells with a labelled anti-CD56 antibody and absence of binding of cells with a labelled anti-CD3 antibody, e.g., as set forth in Example 10 and/or FIG. 14. In various embodiments, CD20+ B cells and/or a CD20+ phenotype can refer to cells found to express CD20, e.g., based on binding of cells with a labelled anti-CD20 antibody, e.g., as set forth in Example 10 and/or FIG. 14. In various embodiments, labeling can be determined by any of a variety of methods known in the art, including without limitation by relative presence of a label, such as a fluorescence of a fluorescence label. In various embodiments, labeling can be measured by techniques including methods such as fluorescence-activated cell sorting (FACS). Accordingly, in various embodiments, monocytes can refer to a population of cells that are CD11+/CD14+ cells and/or determined to have a CD11+/CD14+ phenotype. In various embodiments, T cells can refer to a population of cells that are CD3+ cells and/or determined to have a CD3+ phenotype. In various embodiments, NK cells can refer to a population of cells that are CD3−/CD56+ cells and/or determined to have a CD3−/CD56+ phenotype. In various embodiments, B cells can refer to a population of cells that are CD20+ cells and/or determined to have a CD20+ phenotype.

In various embodiments, vectors and genomes of the present disclosure that can infect and/or transduce, and/or selectively target, CD11+/CD14+ monocytes are vectors and genomes of Ad1, Ad16, Ad34, and/or Ad35 serotype. In various embodiments, vectors and genomes of the present disclosure that can infect and/or transduce, and/or selectively target, CD3+ T cells are vectors and genomes of Ad5, Ad16, Ad34, and/or Ad35 serotype. In various embodiments, vectors and genomes of the present disclosure that can infect and/or transduce, and/or selectively target, CD3−/CD56+NK cells are vectors and genomes of Ad11, Ad16, Ad34, and/or Ad35 serotype. In various embodiments, vectors and genomes of the present disclosure that can infect and/or transduce, and/or selectively target, CD20+ B cells are vectors and genomes of Ad16 serotype.

In various embodiments, vectors and genomes of the present disclosure that can infect and/or transduce, and/or selectively target, CD11+/CD14+ monocytes are vectors and genomes of Ad11, Ad34, and/or Ad35 serotype. In various embodiments, vectors and genomes of the present disclosure that can infect and/or transduce, and/or selectively target, CD3+ T cells are vectors and genomes of Ad34 and/or Ad35 serotype. In various embodiments, vectors and genomes of the present disclosure that can infect and/or transduce, and/or selectively target, CD3−/CD56+NK cells are vectors and genomes of Ad11, Ad34, and/or Ad35 serotype.

In various embodiments, vectors and genomes of the present disclosure can infect and/or transduce, and/or selectively target, monocytes, T cells, NK cells, and/or B cells. In various embodiments, vectors and genomes of the present disclosure that can infect and/or transduce, and/or selectively target, monocytes are vectors and genomes of Ad11, Ad16, Ad34, and/or Ad35 serotype. In various embodiments, vectors and genomes of the present disclosure that can infect and/or transduce, and/or selectively target, T cells are vectors and genomes of Ad5, Ad16, Ad34, and/or Ad35 serotype. In various embodiments, vectors and genomes of the present disclosure that can infect and/or transduce, and/or selectively target, NK cells are vectors and genomes of Ad11, Ad16, Ad34, and/or Ad35 serotype. In various embodiments, vectors and genomes of the present disclosure that can infect and/or transduce, and/or selectively target, B cells are vectors and genomes of Ad16 serotype.

In various embodiments, vectors and genomes of the present disclosure can infect and/or transduce, and/or selectively target, monocytes, T cells, and/or NK cells. In various embodiments, vectors and genomes of the present disclosure that can infect and/or transduce, and/or selectively target, monocytes are vectors and genomes of Ad11, Ad34, and/or Ad35 serotype. In various embodiments, vectors and genomes of the present disclosure that can infect and/or transduce, and/or selectively target, T cells are vectors and genomes of Ad34 and/or Ad35 serotype. In various embodiments, vectors and genomes of the present disclosure that can infect and/or transduce, and/or selectively target, NK cells are vectors and genomes of Ad11, Ad34, and/or Ad35 serotype.

In various embodiments, vectors and genomes of the present disclosure that can infect and/or transduce, and/or selectively target, CD11+/CD14+ monocytes are vectors and genomes of Ad5, Ad7, Ad11, Ad16, Ad34, and/or Ad35 serotype. In various embodiments, vectors and genomes of the present disclosure that can infect and/or transduce, and/or selectively target, CD3+ T cells are vectors and genomes of Ad5, Ad7, Ad1, Ad16, Ad34, and/or Ad35 serotype. In various embodiments, vectors and genomes of the present disclosure that can infect and/or transduce, and/or selectively target, CD3−/CD56+NK cells are vectors and genomes of Ad5, Ad7, Ad1, Ad16, Ad34, and/or Ad35 serotype. In various embodiments, vectors and genomes of the present disclosure that can infect and/or transduce, and/or selectively target, CD20+ B cells are vectors and genomes of Ad5, Ad7, Ad1, Ad16, Ad34, and/or Ad35 serotype.

In various embodiments, vectors and genomes of the present disclosure that can infect and/or transduce, and/or selectively target, monocytes are vectors and genomes of Ad5, Ad7, Ad11, Ad16, Ad34, and/or Ad35 serotype. In various embodiments, vectors and genomes of the present disclosure that can infect and/or transduce, and/or selectively target, T cells are vectors and genomes of Ad5, Ad7, Ad1, Ad16, Ad34, and/or Ad35 serotype. In various embodiments, vectors and genomes of the present disclosure that can infect and/or transduce, and/or selectively target, NK cells are vectors and genomes of Ad5, Ad7, Ad11, Ad16, Ad34, and/or Ad35 serotype. In various embodiments, vectors and genomes of the present disclosure that can infect and/or transduce, and/or selectively target, B cells are vectors and genomes of Ad5, Ad7, Ad1, Ad16, Ad34, and/or Ad35 serotype.

### III. Dosages, Formulations, and Administration

A vector can be formulated such that it is pharmaceutically acceptable for administration to cells or animals, e.g., to humans. A vector may be administered in vitro, ex vivo, or in vivo. The adenoviral vectors described herein can be formulated for administration to a subject. Formulations include an adenoviral vector encoding a therapeutic agent and one or more pharmaceutically acceptable carriers.

As disclosed herein, a vector can be in any form known in the art. Such forms include, e.g., liquid, semi-solid and solid dosage forms, such as liquid solutions (e.g., injectable and infusible solutions), dispersions or suspensions, tablets, pills, powders, liposomes and suppositories.

Selection or use of any particular form may depend, in part, on the intended mode of administration and therapeutic application. For example, compositions containing a composition intended for systemic or local delivery can be in the form of injectable or infusible solutions. Accordingly, a vector can be formulated for administration by a parenteral mode (e.g., intravenous, subcutaneous, intraperitoneal, or intramuscular injection). As used herein, parenteral administration refers to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intracisternal injection and infusion. A parenteral route of administration can be, for example, administration by injection, transnasal administration, transpulmonary administration, or transcutaneous administration. Administration can be systemic or local by intravenous injection, intramuscular injection, intraperitoneal injection, subcutaneous injection.

In various embodiments, a vector of the present invention can be formulated as a solution, microemulsion, dispersion, liposome, or other ordered structure suitable for stable storage at high concentration. Sterile injectable solutions can be prepared by incorporating a composition described herein in the required amount in an appropriate solvent with one or a combination of ingredients enumerated above, as required, followed by filter sterilization. Generally, dispersions are prepared by incorporating a composition described herein into a sterile vehicle that contains a basic dispersion medium and the required other ingredients from those enumerated above. In the case of sterile powders for the preparation of sterile injectable solutions, methods for preparation include vacuum drying and freeze-drying that yield a powder of a composition described herein plus any additional desired ingredient (see below) from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition a reagent that delays absorption, for example, monostearate salts, and gelatin.

A vector can be administered parenterally in the form of an injectable formulation including a sterile solution or suspension in water or another pharmaceutically acceptable liquid. For example, the vector can be formulated by suitably combining the therapeutic molecule with pharmaceutically acceptable vehicles or media, such as sterile water and physiological saline, vegetable oil, emulsifier, suspension agent, surfactant, stabilizer, flavoring excipient, diluent, vehicle, preservative, binder, followed by mixing in a unit dose form required for generally accepted pharmaceutical practices. The amount of vector included in the pharmaceutical preparations is such that a suitable dose within the designated range is provided. Nonlimiting examples of oily liquid include sesame oil and soybean oil, and it may be combined with benzyl benzoate or benzyl alcohol as a solubilizing agent. Other items that may be included are a buffer such as a phosphate buffer, or sodium acetate buffer, a soothing agent such as procaine hydrochloride, a stabilizer such as benzyl alcohol or phenol, and an antioxidant. The formulated injection can be packaged in a suitable ampule.

In various embodiments, subcutaneous administration can be accomplished by means of a device, such as a syringe, a prefilled syringe, an auto-injector (e.g., disposable or reusable), a pen injector, a patch injector, a wearable injector, an ambulatory syringe infusion pump with subcutaneous infusion sets, or other device for subcutaneous injection.

In some embodiments, a vector described herein can be therapeutically delivered to a subject by way of local administration. As used herein, “local administration” or “local delivery,” can refer to delivery that does not rely upon transport of the vector or vector to its intended target tissue or site via the vascular system. For example, the vector may be delivered by injection or implantation of the composition or agent or by injection or implantation of a device containing the composition or agent. In certain embodiments, following local administration in the vicinity of a target tissue or site, the composition or agent, or one or more components thereof, may diffuse to an intended target tissue or site that is not the site of administration.

In some embodiments, compositions provided herein are present in unit dosage form, which unit dosage form can be suitable for self-administration. Such a unit dosage form may be provided within a container, typically, for example, a vial, cartridge, prefilled syringe or disposable pen. A doser such as the doser device described in U.S. Pat. No. 6,302,855, may also be used, for example, with an injection system as described herein.

Pharmaceutical forms of vector formulations suitable for injection can include sterile aqueous solutions or dispersions. A formulation can be sterile and must be fluid to allow proper flow in and out of a syringe. A formulation can also be stable under the conditions of manufacture and storage. A carrier can be a solvent or dispersion medium containing, for example, water and saline or buffered aqueous solutions. Preferably, isotonic agents, for example, sugars or sodium chloride can be used in the formulations.

A suitable dose of a vector described herein can depend on a variety of factors including, e.g., the age, sex, and weight of a subject to be treated, the condition or disease to be treated, and the particular vector used. Other factors affecting the dose administered to the subject include, e.g., the type or severity of the condition or disease. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. A suitable means of administration of a vector can be selected based on the condition or disease to be treated and upon the age and condition of a subject. Dose and method of administration can vary depending on the weight, age, condition, and the like of a patient, and can be suitably selected as needed by those skilled in the art. A specific dosage and treatment regimen for any particular subject can be adjusted based on the judgment of a medical practitioner.

In various instances, a vector can be formulated to include a pharmaceutically acceptable carrier or excipient. Examples of pharmaceutically acceptable carriers include, without limitation, any and all solvents, dispersion media, coatings, antibacterial and antifungal agents, isotonic and absorption delaying agents, and the like that are physiologically compatible. Compositions of the present invention can include a pharmaceutically acceptable salt, e.g., an acid addition salt or a base addition salt.

Exemplary generally used pharmaceutically acceptable carriers include any and all absorption delaying agents, antioxidants, binders, buffering agents, bulking agents or fillers, chelating agents, coatings, disintegration agents, dispersion media, gels, isotonic agents, lubricants, preservatives, salts, solvents or co-solvents, stabilizers, surfactants, and/or delivery vehicles.

In various embodiments, a composition including a vector as described herein, e.g., a sterile formulation for injection, can be formulated in accordance with conventional pharmaceutical practices using distilled water for injection as a vehicle. For example, physiological saline or an isotonic solution containing glucose and other supplements such as D-sorbitol, D-mannose, D-mannitol, and sodium chloride may be used as an aqueous solution for injection, optionally in combination with a suitable solubilizing agent, for example, alcohol such as ethanol and polyalcohol such as propylene glycol or polyethylene glycol, and a nonionic surfactant such as polysorbate 80™, HCO-50 and the like.

The formulations disclosed herein can be formulated for administration by, for example, injection. For injection, formulation can be formulated as aqueous solutions, such as in buffers including Hanks' solution, Ringer's solution, or physiological saline, or in culture media, such as Iscove's Modified Dulbecco's Medium (IMDM). The aqueous solutions can include formulatory agents such as suspending, stabilizing, and/or dispersing agents. Alternatively, the formulation can be in lyophilized and/or powder form for constitution with a suitable vehicle, e.g., sterile pyrogen-free water, before use.

Any formulation disclosed herein can advantageously include any other pharmaceutically acceptable carriers which include those that do not produce significantly adverse, allergic, or other untoward reactions that outweigh the benefit of administration. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety, and purity standards as required by US FDA Office of Biological Standards and/or other relevant foreign regulatory agencies.

Therapeutically effective amounts of adenoviral vector associated with a therapeutic gene can include doses ranging from, for example, 1×10⁷ to 50×10⁸ infection units (IU) or from 5×10⁷ to 20×10⁸ IU. In other examples, a dose can include 5×10⁷ IU, 6×10⁷ IU, 7×10⁷ IU, 8×10⁷ IU, 9×10⁷ IU, 1×10⁸ IU, 2×10⁸ IU, 3×10⁸ IU, 4×10⁸ IU, 5×10⁸ IU, 6×10⁸ IU, 7×10⁸ IU, 8×10⁸ IU, 9×10⁸ IU, 10×10⁸ IU, or more. In particular embodiments, a therapeutically effective amount of an adenoviral vector associated with a therapeutic gene includes 4×10⁸ IU. In particular embodiments, a therapeutically effective amount of an adenoviral vector associated with a therapeutic gene can be administered subcutaneously or intravenously. In particular embodiments, a therapeutically effective amount of an adenoviral vector associated with a therapeutic gene can be administered following administration with one or more mobilization factors.

In various embodiments of the present disclosure, an in vivo gene therapy includes administration of at least one viral gene therapy vector to a subject in combination with at least one immune suppression regimen. In an in vivo gene therapy including more than one vector species, such as a first vector that is a supported viral gene therapy vector in combination with a second vector that is a support vector, the first vector and the second vector can be administered in a single formulation or dosage form or in two separate formulations or dosage forms. In various embodiments, the first and second vectors can be administered at the same time or at different times, e.g., during the same one-hour period or during non-overlapping one-hour periods. In various embodiments, the first and second vectors can be administered at the same time or at different times, e.g., on the same day or on different days. In various embodiments, the first and second vectors can be administered at the same dosage or at different dosages, e.g., where the dosage is measured as the total number of viral particles or as a number of viral particles per kilogram of the subject. In various embodiments, the first and second vectors can be administered in a pre-defined ratio. In various embodiments, the ratio is in the range of 2:1 to 1:2, e.g., 1:1.

In various embodiments, a vector is administered to a subject in a single total dose on a single day. In various embodiments, a vector is administered in two, three, four, or more unit doses that together constitute a total dose. In various embodiments, one unit dose of a vector is administered to a subject per day on each of one, two, three, four, or more consecutive days. In various embodiments, two unit doses of a vector are administered to a subject per day on each of one, two, three, four, or more consecutive days. Accordingly, in various embodiments, a daily dose can refer to the dose of vector received by a subject over the course of a day. In various embodiments, the term day refers to a twenty-four-hour period, such as a twenty-four-hour period from midnight of a first calendar date to midnight of the next calendar date.

In various embodiments, a unit dose, daily dose, or total dose of a vector, such as a viral gene therapy vector or support vector, or the total combined dose of a viral gene therapy vector and a support vector, can be at least 1E8, 5E8, 1E9, 5E9, 1E10, 5E10, 1E11, 5E11, 1E12, 5E12, 1E13, 5E13, 1E14, or 1E15 viral particles per kilogram (vp/kg). In various embodiments, a unit dose, daily dose, or total dose of a vector, such as a viral gene therapy vector or support vector, or the total combined dose of a viral gene therapy vector and a support vector, can fall within a range having a lower bound selected from 1E8, 5E8, 1E9, 5E9, 1E10, 5E10, 1E11, 5E11, 1E12, 5E12, 1E13, 5E13, 1E14, or 1E15 vp/kg and an upper bound selected from 1E8, 5E8, 1E9, 5E9, 1E10, 5E10, 1E11, 5E11, 1E12, 5E12, 1E13, 5E13, 1E14, or 1E15 vp/kg.

In various embodiments, a viral gene therapy vector is administered at a unit dose, daily dose, or total dose of at least 1E10, 5E10, 1E11, 5E11, 1E12, 5E12, 1E13, 5E13, 1E14, or 1E15 vp/kg and a support vector is administered at a unit dose, daily dose, or total dose of at least 1E8, 5E8, 1E9, 5E9, 1E10, 5E10, 1E11, and 5E11 vp/kg, optionally where the unit dose, daily dose, or total dose of the viral gene therapy vector is within a range having a lower bound selected from 1E10, 5E10, 1E11, 5E11, 1E12, and 5E12, vp/kg and an upper bound selected from 1E11, 5E11, 1E12, 5E12, 1E13, 5E13, 1E14, and 1E15 vp/kg, and/or where the unit dose, daily dose, or total dose of the support vector is within a range having a lower bound selected from 1E8, 5E8, 1E9, 5E9, 1E10, and 5E10 vp/kg and an upper bound selected from 1E9, 5E9, 1E10, 5E10, 1E11, and 5E11 vp/kg.

In various embodiments, a support vector is administered at a unit dose, daily dose, or total dose of at least 1E10, 5E10, 1E11, 5E11, 1E12, 5E12, 1E13, 5E13, 1E14, or 1E15 vp/kg and a supported viral gene therapy vector is administered at a unit dose, daily dose, or total dose of at least 1E8, 5E8, 1E9, 5E9, 1E10, 5E10, 1E11, and 5E11 vp/kg, optionally where the unit dose, daily dose, or total dose of the support vector is within a range having a lower bound selected from 1E10, 5E10, 1E11, 5E11, 1E12, and 5E12, vp/kg and an upper bound selected from 1E11, 5E11, 1E12, 5E12, 1E13, 5E13, 1E14, and 1E15 vp/kg, and/or where the unit dose, daily dose, or total dose of the supported viral gene therapy vector is within a range having a lower bound selected from 1E8, 5E8, 1E9, 5E9, 1E10, and 5E10 vp/kg and an upper bound selected from 1E9, 5E9, 1E10, 5E10, 1E11, and 5E11 vp/kg. In various embodiments, a supported viral gene therapy vector and a support vector are administered in a pre-defined ratio. In various embodiments, the ratio is in the range of 2:1 to 1:2, e.g., 1:1.

### IV. Applications

Methods and compositions provided herein are disclosed at least in part for use in in vivo gene therapy. However, for the avoidance of doubt, the present disclosure expressly includes the use of compositions and methods provided herein for ex vivo engineering of cells and/or tissues, as well as in vitro uses including the engineering of cells and/or tissues for research purposes. Gene therapy includes use of a vector, genome, or system of the present disclosure in a method of introducing exogenous DNA into a host cell (such as a target cell) and/or a nucleic acid (such as a target nucleic acid, such as a target genome, e.g., the genome of a target cell), which introducing of exogenous DNA can be referred to as genetic modification of the host cell or nucleic acid. Gene therapy can therefore be referred to herein, e.g., as a method of genetically modifying a host cell or nucleic acid. The present disclosure includes description and exemplification of compositions and methods relating to in vivo, in vitro, and ex vivo therapy and those of skill in the art will appreciate that various methods and compositions provided herein are generally applicable to introduction of a nucleic acid payload into a subject, e.g., a host or target cell. Because such compositions and methods are of general utility, e.g., in gene therapy, they are useful both as tools in gene therapy in general and in various particular conditions, including those provided herein.

### IV(a). In Vivo Gene Therapy

Treatments using in vivo gene therapy, which includes the direct delivery of a viral vector to a patient, have been explored. In vivo gene therapy is an attractive approach because it may not require any genotoxic conditioning (or could require less genotoxic conditioning) nor ex vivo cell processing and thus could be adopted at many institutions worldwide, including those in developing countries, as the therapy could be administered through an injection, similar to what is already done worldwide for the delivery of vaccines. In various embodiments, methods of in vivo gene therapy with adenoviral vectors of the present disclosure can include one or more steps of (i) target cell mobilization, (ii) immunosuppression, (iii) administration of a vector, genome, system or formulation provided herein, and/or (iv) selection of transduced cells and/or cells that have integrated an integration element of a payload of an adenoviral vector or genome.

In various embodiments, methods and compositions disclosed herein can be used for treating subjects (humans, veterinary animals (dogs, cats, reptiles, birds, etc.), livestock (horses, cattle, goats, pigs, chickens, etc.), and research animals (monkeys, rats, mice, fish, etc.). Treating subjects includes delivering therapeutically effective amounts of one or more vectors, genomes, or systems of the present disclosure. Therapeutically effective amounts include those that provide effective amounts, prophylactic treatments, and/or therapeutic treatments.

Vectors disclosed herein can be administered in coordination with mobilization factors. In certain embodiments, adenoviral vector compositions described herein can be administered in concert with HSPC mobilization. In particular embodiments, administration of adenoviral donor vector occurs concurrently with administration of one or more mobilization factors. In particular embodiments, administration of adenoviral donor vector follows administration of one or more mobilization factors. In particular embodiments, administration of adenoviral donor vector follows administration of a first one or more mobilization factors and occurs concurrently with administration of a second one or more mobilization factors. Agents for HSPC mobilization include, for example, granulocyte-colony stimulating factor (G-CSF), granulocyte macrophage colony stimulating factor (GM-CSF), AMD3100, SCF, S-CSF, a CXCR4 antagonist, a CXCR2 agonist, and Gro-Beta (GRO-β). In various embodiments, a CXCR4 antagonist is AMD3100 and/or a CXCR2 agonist is GRO-β.

G-CSF is a cytokine whose functions in HSPC mobilization can include the promotion of granulocyte expansion and both protease-dependent and independent attenuation of adhesion molecules and disruption of the SDF-1/CXCR4 axis. In particular embodiments, any commercially available form of G-CSF known to one of ordinary skill in the art can be used in the methods and compositions as disclosed herein, for example, Filgrastim (Neupogen®, Amgen Inc., Thousand Oaks, CA) and PEGylated Filgrastim (Pegfilgrastim, NEULASTA®, Amgen Inc., Thousand Oaks, CA).

GM-CSF is a monomeric glycoprotein also known as colony-stimulating factor 2 (CSF2) that functions as a cytokine and is naturally secreted by macrophages, T cells, mast cells, natural killer cells, endothelial cells, and fibroblasts. In particular embodiments, any commercially available form of GM-CSF known to one of ordinary skill in the art can be used in the methods and compositions as disclosed herein, for example, Sargramostim (Leukine, Bayer Healthcare Pharmaceuticals, Seattle, WA) and molgramostim (Schering-Plough, Kenilworth, NJ).

AMD3100 (MOZOBIL™, PLERIXAFOR™; Sanofi-Aventis, Paris, France), a synthetic organic molecule of the bicyclam class, is a chemokine receptor antagonist and reversibly inhibits SDF-1 binding to CXCR4, promoting HSPC mobilization. AMD3100 is approved to be used in combination with G-CSF for HSPC mobilization in patients with myeloma and lymphoma.

SCF, also known as KIT ligand, KL, or steel factor, is a cytokine that binds to the c-kit receptor (CD117). SCF can exist both as a transmembrane protein and a soluble protein. This cytokine plays an important role in hematopoiesis, spermatogenesis, and melanogenesis. In particular embodiments, any commercially available form of SCF known to one of ordinary skill in the art can be used in the methods and compositions as disclosed herein, for example, recombinant human SCF (Ancestim, STEMGEN®, Amgen Inc., Thousand Oaks, CA).

Chemotherapy used in intensive myelosuppressive treatments also mobilizes HSPCs to the peripheral blood as a result of compensatory neutrophil production following chemotherapy-induced aplasia. In particular embodiments, chemotherapeutic agents that can be used for mobilization of HSPCs include cyclophosphamide, etoposide, ifosfamide, cisplatin, and cytarabine.

Additional agents that can be used for cell mobilization include: CXCL12/CXCR4 modulators (e.g., CXCR4 antagonists: POL6326 (Polyphor, Allschwil, Switzerland), a synthetic cyclic peptide which reversibly inhibits CXCR4; BKT-140 (4F-benzoyl-TN14003; Biokine Therapeutics, Rehovit, Israel); TG-0054 (Taigen Biotechnology, Taipei, Taiwan); CXCL12 neutralizer NOX-A12 (NOXXON Pharma, Berlin, Germany) which binds to SDF-1, inhibiting its binding to CXCR4); Sphingosine-1-phosphate (S1P) agonists (e.g., SEW2871, Juarez et al. Blood 119: 707-716, 2012); vascular cell adhesion molecule-1 (VCAM) or very late antigen 4 (VLA-4) inhibitors (e.g., Natalizumab, a recombinant humanized monoclonal antibody against α4 subunit of VLA-4 (Zohren et al. Blood 111: 3893-3895, 2008); BIO5192, a small molecule inhibitor of VLA-4 (Ramirez et al. Blood 114: 1340-1343, 2009)); parathyroid hormone (Brunner et al. Exp Hematol. 36: 1157-1166, 2008); proteasome inhibitors (e.g., Bortezomib, Ghobadi et al. ASH Annual Meeting Abstracts. p. 583, 2012); Groβ, a member of CXC chemokine family which stimulates chemotaxis and activation of neutrophils by binding to the CXCR2 receptor (e.g., SB-251353, King et al. Blood 97: 1534-1542, 2001); stabilization of hypoxia inducible factor (HIF) (e.g., FG-4497, Forristal et al. ASH Annual Meeting Abstracts. p. 216, 2012); Firategrast, an α4β1 and α4=β7 integrin inhibitor (α4β1/7) (Kim et al. Blood 128: 2457-2461, 2016); Vedolizumab, a humanized monoclonal antibody against the α4β7 integrin (Rosario et al. Clin Drug Investig 36: 913-923, 2016); and BOP (N-(benzenesulfonyl)-L-prolyl-L-O-(1-pyrrolidinylcarbonyl) tyrosine) which targets integrins α9β1/α4β1 (Cao et al. Nat Commun 7: 11007, 2016). Additional agents that can be used for HSPC mobilization are described in, for example, Richter R et al. Transfus Med Hemother 44:151-164, 2017, Bendall & Bradstock, Cytokine & Growth Factor Reviews 25: 355-367, 2014, WO 2003043651, WO 2005017160, WO 2011069336, U.S. Pat. Nos. 5,637,323, 7,288,521, 9,782,429, US 2002/0142462, and US 2010/02268.

In particular embodiments, a therapeutically effective amount of G-CSF includes 0.1 μg/kg to 100 μg/kg. In particular embodiments, a therapeutically effective amount of G-CSF includes 0.5 μg/kg to 50 μg/kg. In particular embodiments, a therapeutically effective amount of G-CSF includes 0.5 μg/kg, 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 11 μg/kg, 12 μg/kg, 13 μg/kg, 14 μg/kg, 15 μg/kg, 16 μg/kg, 17 μg/kg, 18 μg/kg, 19 μg/kg, 20 μg/kg, or more. In particular embodiments, a therapeutically effective amount of G-CSF includes 5 μg/kg. In particular embodiments, G-CSF can be administered subcutaneously or intravenously. In particular embodiments, G-CSF can be administered for 1 day, 2 consecutive days, 3 consecutive days, 4 consecutive days, 5 consecutive days, or more. In particular embodiments, G-CSF can be administered for 4 consecutive days. In particular embodiments, G-CSF can be administered for 5 consecutive days. In particular embodiments, as a single agent, G-CSF can be used at a dose of 10 μg/kg subcutaneously daily, initiated 3, 4, 5, 6, 7, or 8 days before adenoviral delivery. In particular embodiments, G-CSF can be administered as a single agent followed by concurrent administration with another mobilization factor. In particular embodiments, G-CSF can be administered as a single agent followed by concurrent administration with AMD3100. In particular embodiments, a treatment protocol includes a 5 day treatment where G-CSF can be administered on day 1, day 2, day 3, and day 4 and on day 5, G-CSF and AMD3100 are administered 6 to 8 hours prior to adenoviral administration.

Therapeutically effective amounts of GM-CSF to administer can include doses ranging from, for example, 0.1 to 50 μg/kg or from 0.5 to 30 μg/kg. In particular embodiments, a dose at which GM-CSF can be administered includes 0.5 μg/kg, 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 11 μg/kg, 12 μg/kg, 13 μg/kg, 14 μg/kg, 15 μg/kg, 16 μg/kg, 17 μg/kg, 18 μg/kg, 19 μg/kg, 20 μg/kg, or more. In particular embodiments, GM-CSF can be administered subcutaneously for 1 day, 2 consecutive days, 3 consecutive days, 4 consecutive days, 5 consecutive days, or more. In particular embodiments, GM-CSF can be administered subcutaneously or intravenously. In particular embodiments, GM-CSF can be administered at a dose of 10 μg/kg subcutaneously daily initiated 3, 4, 5, 6, 7, or 8 days before adenoviral delivery. In particular embodiments, GM-CSF can be administered as a single agent followed by concurrent administration with another mobilization factor. In particular embodiments, GM-CSF can be administered as a single agent followed by concurrent administration with AMD3100. In particular embodiments, a treatment protocol includes a 5 day treatment where GM-CSF can be administered on day 1, day 2, day 3, and day 4 and on day 5, GM-CSF and AMD3100 are administered 6 to 8 hours prior to adenoviral administration. A dosing regimen for Sargramostim can include 200 μg/m², 210 μg/m², 220 μg/m², 230 μg/m², 240 μg/m², 250 μg/m², 260 μg/m², 270 μg/m², 280 μg/m², 290 μg/m², 300 μg/m², or more. In particular embodiments, Sargramostim can be administered for 1 day, 2 consecutive days, 3 consecutive days, 4 consecutive days, 5 consecutive days, or more. In particular embodiments, Sargramostim can be administered subcutaneously or intravenously. In particular embodiments, a dosing regimen for Sargramostim can include 250 μg/m²/day intravenous or subcutaneous and can be continued until a targeted cell amount is reached in the peripheral blood or can be continued for 5 days. In particular embodiments, Sargramostim can be administered as a single agent followed by concurrent administration with another mobilization factor. In particular embodiments, Sargramostim can be administered as a single agent followed by concurrent administration with AMD3100. In particular embodiments, a treatment protocol includes a 5 day treatment where Sargramostim can be administered on day 1, day 2, day 3, and day 4 and on day 5, Sargramostim and AMD3100 are administered 6 to 8 hours prior to adenoviral administration.

In particular embodiments, a therapeutically effective amount of AMD3100 includes 0.1 mg/kg to 100 mg/kg. In particular embodiments, a therapeutically effective amount of AMD3100 includes 0.5 mg/kg to 50 mg/kg. In particular embodiments, a therapeutically effective amount of AMD3100 includes 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, or more. In particular embodiments, a therapeutically effective amount of AMD3100 includes 4 mg/kg. In particular embodiments, a therapeutically effective amount of AMD3100 includes 5 mg/kg. In particular embodiments, a therapeutically effective amount of AMD3100 includes 10 μg/kg to 500 μg/kg or from 50 μg/kg to 400 μg/kg. In particular embodiments, a therapeutically effective amount of AMD3100 includes 100 μg/kg, 150 μg/kg, 200 μg/kg, 250 μg/kg, 300 μg/kg, 350 μg/kg, or more. In particular embodiments, AMD3100 can be administered subcutaneously or intravenously. In particular embodiments, AMD3100 can be administered subcutaneously at 160-240 μg/kg 6 to 11 hours prior to adenoviral delivery. In particular embodiments, a therapeutically effective amount of AMD3100 can be administered concurrently with administration of another mobilization factor. In particular embodiments, a therapeutically effective amount of AMD3100 can be administered following administration of another mobilization factor. In particular embodiments, a therapeutically effective amount of AMD3100 can be administered following administration of G-CSF. In particular embodiments, a treatment protocol includes a 5-day treatment where G-CSF is administered on day 1, day 2, day 3, and day 4 and on day 5, G-CSF and AMD3100 are administered 6 to 8 hours prior to adenoviral injection.

Therapeutically effective amounts of SCF to administer can include doses ranging from, for example, 0.1 to 100 μg/kg/day or from 0.5 to 50 μg/kg/day. In particular embodiments, a dose at which SCF can be administered includes 0.5 μg/kg/day, 1 μg/kg/day, 2 μg/kg/day, 3 μg/kg/day, 4 μg/kg/day, 5 μg/kg/day, 6 μg/kg/day, 7 μg/kg/day, 8 μg/kg/day, 9 μg/kg/day, 10 μg/kg/day, 11 μg/kg/day, 12 μg/kg/day, 13 μg/kg/day, 14 μg/kg/day, 15 μg/kg/day, 16 μg/kg/day, 17 μg/kg/day, 18 μg/kg/day, 19 μg/kg/day, 20 μg/kg/day, 21 μg/kg/day, 22 μg/kg/day, 23 μg/kg/day, 24 μg/kg/day, 25 μg/kg/day, 26 μg/kg/day, 27 μg/kg/day, 28 μg/kg/day, 29 μg/kg/day, 30 μg/kg/day, or more. In particular embodiments, SCF can be administered for 1 day, 2 consecutive days, 3 consecutive days, 4 consecutive days, 5 consecutive days, or more. In particular embodiments, SCF can be administered subcutaneously or intravenously. In particular embodiments, SCF can be injected subcutaneously at 20 μg/kg/day. In particular embodiments, SCF can be administered as a single agent followed by concurrent administration with another mobilization factor. In particular embodiments, SCF can be administered as a single agent followed by concurrent administration with AMD3100. In particular embodiments, a treatment protocol includes a 5 day treatment where SCF can be administered on day 1, day 2, day 3, and day 4 and on day 5, SCF and AMD3100 are administered 6 to 8 hours prior to adenoviral administration.

In particular embodiments, growth factors GM-CSF and G-CSF can be administered to mobilize HSPC in the bone marrow niches to the peripheral circulating blood to increase the fraction of HSPCs circulating in the blood. In particular embodiments, mobilization can be achieved with administration of G-CSF/Filgrastim (Amgen) and/or AMD3100 (Sigma). In particular embodiments, mobilization can be achieved with administration of GM-CSF/Sargramostim (Amgen) and/or AMD3100 (Sigma). In particular embodiments, mobilization can be achieved with administration of SCF/Ancestim (Amgen) and/or AMD3100 (Sigma). In particular embodiments, administration of G-CSF/Filgrastim precedes administration of AMD3100. In particular embodiments, administration of G-CSF/Filgrastim occurs concurrently with administration of AMD3100. In particular embodiments, administration of G-CSF/Filgrastim precedes administration of AMD3100, followed by concurrent administration of G-CSF/Filgrastim and AMD3100. US 20140193376 describes mobilization protocols utilizing a CXCR4 antagonist with a S1P receptor 1 (S1PR1) modulator agent. US 20110044997 describes mobilization protocols utilizing a CXCR4 antagonist with a vascular endothelial growth factor receptor (VEGFR) agonist.

Adenoviral vectors (e.g. Ad3, 5, 7, 11, 14, 16, 21, 34, 35, 37, or 50 vectors) are exemplary of vectors that can be administered in concert with HSPC mobilization. In particular embodiments, administration of an adenoviral vector occurs concurrently with administration of one or more mobilization factors. In particular embodiments, administration of an Adenoviral vector follows administration of one or more mobilization factors. In particular embodiments, administration of an Adenoviral vector follows administration of a first one or more mobilization factors and occurs concurrently with administration of a second one or more mobilization factors.

In particular embodiments, an HSC enriching agent, such as a CD19 immunotoxin or 5-FU can be administered to enrich for HSPCs. CD19 immunotoxin can be used to deplete all CD19 lineage cells, which accounts for 30% of bone marrow cells. Depletion encourages exit from the bone marrow. By forcing HSPCs to proliferate (whether via, e.g., CD19 immunotoxin of 5-FU), this stimulates their differentiation and exit from the bone marrow and increases transgene marking in peripheral blood cells.

Therapeutically effective amounts of HSC mobilization factors and/or HSC enriching agents can be administered through any appropriate administration route such as by, injection, infusion, perfusion, and more particularly by administration by one or more of bone marrow, intravenous, intradermal, intraarterial, intranodal, intralymphatic, intraperitoneal injection, infusion, or perfusion).

In particular embodiments, methods of the present disclosure can include selection for cells modified to express a selection marker (e.g., a mutant form of MGMT that is resistant to inactivation by 6-BG, but retains the ability to repair DNA damage). For example, particular embodiments include regimens that combine mobilization (e.g., a mobilization protocol described herein) with administration of an adenoviral vector described herein and administration BCNU or benzylguanine and temozolomide in the case of an adenoviral vector including a MGMT^{P140K} selection marker. In particular embodiments, the in vivo selection marker can include MGMT^{P140K} as described in Olszko et al., Gene Therapy 22: 591-595, 2015. Thus, selection for cells that express MGMT^{P140K} can select for transduced cells and/or contribute to therapeutic efficacy.

Adenoviral vectors can be administered concurrently with or following administration of one or more immunosuppression agents or immunosuppression regimens.

### IV(B). In Vitro and Ex Vivo Gene Therapy

In vitro gene therapy includes use of a vector, genome, or system of the present disclosure in a method of introducing exogenous DNA into a host cell (such as a target cell), system (e.g., a plurality of cells including one or more target and/or host cells), and/or a nucleic acid (such as a target nucleic acid, such as a target genome), where the host cell, system, or nucleic acid is not present in a multicellular organism (e.g., in a laboratory). In some embodiments, a target cell, system, or nucleic acid is derived (e.g., as a biological sample or portion thereof) from a multicellular organism, such as a mammal (e.g., a mouse, rat, human, or non-human primate). In various embodiments, a system can include a plurality of cell types, including for example a plurality of hematopoietic cell types. In vitro engineering of a cell derived from a multicellular organism can be referred to as ex vivo engineering, and can be used in ex vivo therapy. In various embodiments, methods and compositions of the present disclosure are utilized, e.g., as disclosed herein, to modify a target cell or nucleic acid derived from a first multicellular organism and the engineered target cell or nucleic acid is then administered to a second multicellular organism, such as a mammal (e.g., a mouse, rat, human, or non-human primate), e.g., in a method of adoptive cell therapy. In some instances, the first and second organisms are the same single subject organism. Return of in vitro engineered material to a subject from which the material was derived can be an autologous therapy. In some instances, the first and second organisms are different organisms (e.g., two organisms of the same species, e.g., two mice, two rats, two humans, or two non-human primates of the same species). Transfer of engineered material derived from a first subject to a second different subject can be an allogeneic therapy.

Ex vivo cell therapies can include isolation of hematopoietic cells (e.g., stem, progenitor or differentiated cells) from a donor (e.g., a mammalian donor, e.g., a human donor) such as a patient or a normal and/or healthy donor, expansion of isolated cells ex vivo—with or without genetic engineering—and administration of the cells to a subject to establish a transient or stable graft of the infused cells and/or their progeny. Such ex vivo approaches can be used, for example, to treat an inherited, infectious or neoplastic disease, to regenerate a tissue or to deliver a therapeutic agent to a disease site. In various ex vivo therapies there is no direct exposure of the subject to the gene transfer vector, and the target cells of transduction can be selected, expanded and/or differentiated, before or after any genetic engineering, to improve efficacy and safety.

Ex vivo therapies include hematopoietic cell transplantation. Autologous hematopoietic cell gene therapy represents a therapeutic option for several monogenic diseases of the blood and the immune system as well as for storage disorders, and it may become a first-line treatment option for selected disease conditions.

Applications of ex-vivo therapy include reconstituting dysfunctional cell lineages. For inherited diseases characterized by a defective or absent cell lineage, the lineage can be regenerated by functional progenitor cells, derived either from normal donors or from autologous cells that have been subjected to ex vivo gene transfer to correct the deficiency. An example is provided by SCIDs, in which a deficiency in any one of several genes blocks the development of mature lymphoid cells. Transplantation of non-manipulated normal donor hematopoietic cells, which can in various embodiments allow generation of donor-derived functional hematopoietic cells of various lineages in the host, represents a therapeutic option for SCIDs, as well as many other diseases that affect the blood and immune system. Autologous hematopoietic cell gene therapy, which can include engineering of a target hematopoietic cell population and, similarly to allogenic hematopoietic cell transplantation, can provide a steady supply of functional hematopoietic cells (e.g., progeny of engineered hematopoietic stem and/or progenitor cells), may have several advantages, including reduced risk of graft versus host disease (GvHD), reduced risk of graft rejection, and reduced need for post-transplant immunosuppression.

Applications of ex-vivo therapy include augmenting therapeutic gene dosage. In some applications, hematopoietic cell gene therapy may augment the therapeutic efficacy of allogenic hematopoietic cell transplantation. Therapeutic gene dosage can be engineered to supra-normal levels in transplanted cells.

Applications of ex-vivo therapy include introducing novel function and targeting gene therapy. Ex vivo gene therapy can confer a novel function to hematopoietic cells (e.g., one or more particular types of hematopoietic cells) or their progeny, such as establishing drug resistance to allow administration of a high-dose antitumor chemotherapy regime or establishing resistance to a pre-established infection with a virus, such as HIV, or other pathogen by expressing RNA-based agents (for example, ribozymes, RNA decoys, antisense RNA, RNA aptamers and small interfering RNA) and protein-based agents (for example, dominant-negative mutant viral proteins, fusion inhibitors and engineered nucleases that target the pathogen's genome).

### IV(C). Conditions Treatable by Gene Therapy

At least in part because adenoviral vectors of the present disclosure (e.g. Ad3, 5, 7, 11, 14, 16, 21, 34, 35, 37, or 50 vectors) can be used in vivo, in vitro, or ex vivo for modification of host and/or target cells, and further because an adenoviral vector can include payloads encoding a wide variety of expression products, it will be clear from the present specification that various technologies provided herein have broad applicability and can be used to treat a wide variety of conditions. Examples of conditions treatable by administration of an adenoviral vector, genome, or system of the present disclosure include, without limitation genetic conditions (e.g., hemoglobinopathies) and conditions treatable by expression of a therapeutic polypeptide (e.g., cancer).

In various embodiments, methods and compositions of the present disclosure can be used to treat a genetic condition (e.g., a condition arising from and/or caused by a mutation present in the genome of one or more cells of a subject). In various embodiments, methods and compositions of the present disclosure can be used to treat a genetic condition arising from and/or caused by a single point mutation present in the genome of one or more cells of a subject (e.g., a heterozygous or homozygous single point mutation). In various embodiments, methods and compositions of the present disclosure can be used to treat a protein deficiency. In various embodiments, methods and compositions of the present disclosure can be used to treat an enzyme deficiency. In various embodiments, methods and compositions of the present disclosure can be used to treat a blood condition (e.g., a condition characterized by a blood cell abnormality). Examples of genetic (e.g., point mutation) conditions, protein deficiencies, enzyme deficiencies, and/or blood conditions that can be treated by methods and compositions of the present disclosure include adenosine deaminase deficiency (ADA), adrenoleukodystrophy (ALD), agammaglobulinemia, alpha-1 antitrypsin deficiency, congenital amegakaryocytic thrombocytopenia, amyotrophic lateral sclerosis (Lou Gehrig's disease), ataxia telangiectasia, Batten disease, Bernard-Soulier Syndrome, CD40/CD40L deficiency, chronic granulomatous disease, common variable immune deficiency (CVID), congenital thrombotic thrombocytopenic purpura (cTTP), cystic fibrosis, Diamond Blackfan anemia (DBA), DOCK 8 deficiency, dyskeratosis congenital, Fabry disease, Factor V Deficiency, Factor VII Deficiency, Factor X Deficiency, Factor XI Deficiency, Factor XII Deficiency, Factor XIII Deficiency, familial apolipoprotein E deficiency and atherosclerosis (ApoE), familial erythrophagocytic lymphohistiocytosis, Fanconi anemia (FA), Friedreich ataxia, Gaucher disease, Glanzmann thrombasthenia, glucosemia, glycogen storage disease, glycogen storage disease type I (GSDI), Gray Platelet Syndrome, hemophilia, hemophilia A, hemophilia B, hereditary hemochromatosis, Hurler's syndrome, hyper IgM, Hypogammaglobulinemia, Krabbe disease, major histocompatibility complex class II deficiency (MHC-II), maple syrup urine disease, metachromatic leukodystrophy (MLD), mucopolysaccharidoses, mucopolysaccharidosis type I (MPS I), MPS II (Hunter Syndrome), MPS III (Sanfilippo syndrome), MPS IV (Morquio syndrome), MPS V, MPS VI (Maroteaux-Lamy syndrome), MPS VII (sly syndrome), muscular dystrophy, Niemann-Pick disease, Parkinson's disease, paroxysmal nocturnal hemoglobinuria (PNH), pernicious anemia, phenylketonuria (PKU), Pompe disease, pulmonary alveolar proteinosis (PAP), pure red cell aplasia (PRCA), pyruvate kinase deficiency, refractory anemia, Shwachman-Diamond syndrome, selective IgA deficiency, severe aplastic anemia, severe combined immunodeficiency disease (SCID), Severe combined immunodeficiency due to adenosine deaminase deficiency (ADA-SCID), sickle cell anemia, sickle cell disease, sickle cell trait, Tay Sachs, thalassemia, thalassemia intermedia, von Gierke disease, von Willebrand Disease, Wiskott-Aldrich syndrome (WAS), X-linked agammaglobulinemia (XLA), X-linked severe combined immunodeficiency (SCID-X1), Zellweger syndrome, α-mannosidosis, β-mannosidosis, and/or β-thalassemia, β-thalassemia major.

In various embodiments, methods and compositions of the present disclosure can be used to treat an inborn error of metabolism. In various embodiments, methods and compositions of the present disclosure can be used to treat a hyperproliferative condition

In various embodiments, methods and compositions of the present disclosure can be used to treat a cancer (e.g., a cancer characterized by abnormal blood cells). Examples of cancers that can be treated by methods and compositions of the present disclosure include acute lymphoblastic leukemia (ALL), acute myelogenous leukemia (AML), agnogenic myeloid metaplasia, astrocytoma, atypical teratoid rhabdoid tumor, brain and central nervous system (CNS) cancer, breast cancer, carcinosarcoma, chondrosarcoma, chordoma, choroid plexus carcinoma, choroid plexus papilloma, chronic lymphocytic leukemia (CLL), chronic myelogenous leukemia (CML), clear cell sarcoma of soft tissue, diffuse large B-cell lymphoma, ependymoma, epithelioid sarcoma, Ewing sarcoma, extragonadal germ cell tumor, extrarenal rhabdoid tumor, follicular lymphoma, gastrointestinal stromal tumor, glioblastoma, HBV-induced hepatocellular carcinoma, head and neck cancer, Hodgkin's lymphoma, juvenile myelomonocytic leukemia, kidney cancer, lung cancer, lymphoma, malignant rhabdoid tumor, medulloblastoma, melanoma, meningioma, mesothelioma, multiple myeloma, myeloma, neuroglial tumor, non-Hodgkin's lymphoma, not otherwise specified (NOS) sarcoma, oligoastrocytoma, oligodendroglioma, osteosarcoma, ovarian cancer, ovarian clear cell adenocarcinoma, ovarian endometrioid adenocarcinoma, ovarian serous adenocarcinoma, pancreatic cancer, pancreatic ductal adenocarcinoma, pancreatic endocrine tumor, pineoblastoma, prostate cancer, renal cell carcinoma, renal medullary carcinoma, rhabdomyosarcoma, sarcoma, schwannoma, skin squamous cell carcinoma, and/or stem cell cancer.

In various embodiments, methods and compositions of the present disclosure can be used to treat a hemoglobinopathy, red blood cell disorder, platelet disorder, and/or bone marrow disorder (e.g., a bone marrow failure condition).

In various embodiments, methods and compositions of the present disclosure can be used to treat an immune condition (e.g., an autoimmune condition). Examples of immune conditions (e.g., autoimmune conditions) that can be treated by methods and compositions of the present disclosure include acquired immunodeficiency syndrome (AIDS), acquired thrombotic thrombocytopenic purpura (aTTP), an autoimmune hematology, graft versus host disease (GVHD), Grave's Disease, inflammatory bowel disease, Multiple Sclerosis (MS), rheumatoid arthritis, severe aplastic anemia, and systemic lupus erythematosus (SLE).

In various embodiments, methods and compositions of the present disclosure can be used to treat an immunodeficiency (e.g., a primary immune deficiency, secondary immune deficiency, acquired immune deficiency, and/or an immune deficiency caused by trauma), an inflammatory condition, an IgG subclass deficiency, a complement disorders, or a specific antibody deficiency). In various embodiments, methods and compositions of the present disclosure can be used to eliminate or inhibit one or more subsets of lymphocytes (e.g., induce apoptosis in lymphocytes, inhibit lymphocyte activation, inhibit T cell activation, and/or inhibit Th-2 activity, and/or Th-1 activity), eliminate or inhibit autoreactive T cells, improve kinetics and/or clonal diversity of lymphocyte reconstitution, restore normal T lymphocyte development, restore thymic output, induce selective tolerance to an inciting agent, provide function to immune and other blood cells or treat an immune-mediated condition, In various embodiments, methods and compositions of the present disclosure can be used to normalize primary and secondary antibody responses to immunization.

In various embodiments, methods and compositions of the present disclosure can be used to treat and/or prevent an infection. In various embodiments, a compositions of the present disclosure is a vaccine in that it encodes, and/or expresses in one or more cells of a subject, an antigen characteristic of an infectious agent (e.g., a viral or bacterial pathogen). In various embodiments, a method of the present disclosure is a method of vaccination in that it delivers to one or more cells of a subject an antigen characteristic of an infectious agent (e.g., a viral or bacterial pathogen) and/or induces an immune responses against the antigen and/or infectious agent. In various embodiments, a method or composition of the present disclosure delivers (e.g., causes transient expression of) an antigen in a subject. In various embodiments, a method or composition of the present disclosure is used to treat a subject that has the infection. In various embodiments, a method or composition of the present disclosure is used to treat a subject that is at risk of infection.

In various embodiments, a method or composition of the present disclosure delivers to one or more cells of a subject in need thereof a coding sequence that encodes and/or expresses a replacement polypeptide (i.e., a wild type, reference, and/or functional polypeptide that corresponds to a disease variant encoded by the genome of the subject). In various embodiments, a method or composition of the present disclosure delivers to one or more cells of a subject in need thereof an editing system that modifies a nucleic acid of the subject (e.g., a genome of the subject) to express and/or increase expression of a wild type, reference, and/or functional polypeptide, e.g., by correction of a disease mutation present in the nucleic acid of the subject.

Particular examples of conditions that can be treated by methods and compositions of the present disclosure include conditions in which mutation of a globin gene results in expression of an abnormal form of hemoglobin (e.g., as in sickle cell disease (SCD) or hemoglobin C, D, or E disease) or results in reduced production of the α or β polypeptides (and thus an imbalance of the globin chains in the cell). These latter conditions are termed α- or β-thalassemias, depending on which globin chain is impaired. 5% of the world population carries a significant hemoglobin variant with the sickle cell mutation in the b-globin (HBB) gene (a glutamate to valine conversion; historically E6V, contemporaneously E7V) being by far the most common (40% of carriers). The high prevalence and severity of hemoglobin disorders presents a substantial burden, impacting not only the lives of those affected but also health-care systems, since lifelong patient care is costly.

There are two forms of hemoglobin, fetal (HbF), which includes two alpha (α) and two gamma (γ) chains, and adult (HbA), which includes two a and two beta (β) chains. The natural switch from HbF to HbA occurs shortly after birth and is regulated by transcriptional repression of γ globin genes by factors including a master regulator, bcl11a. Critically, a variety of clinical observations demonstrate that the severity of β-hemoglobinopathies such as sickle cell disease and β-thalassemia are ameliorated by increased production of HbF.

In particular embodiments, a therapeutically effective treatment induces or increases expression of HbF, induces or increases production of hemoglobin and/or induces or increases production of β-globin. In particular embodiments, a therapeutically effective treatment improves blood cell function, and/or increases oxygenation of cells.

In various embodiments, the present disclosure includes treatment of a blood disorder using an adenoviral donor vector of the present disclosure that includes a coding nucleic acid sequence that encodes a protein or agent for treatment of the blood disorder. In various embodiments, the blood disorder is thalassemia and the protein is a β-globin or γ-globin protein, or a protein that otherwise partially or completely functionally replaces β-globin or γ-globin. In various embodiments, the blood disorder is hemophilia and the protein is ET3 or a protein that otherwise partially or completely functionally replaces Factor VIII. In various embodiments, the blood disorder is a point mutation disease such as sickle cell anemia, and the agent is a gene editing protein.

ET3 can have or include the following amino acid sequence: SEQ ID NO 210. In various embodiments, a Factor VIII replacement protein can have an amino acid sequence having at least 70%, 75%, 80%, 85%, 90%, 91%, 92%, 93%, 94%, 95%, 96%, 97%, 98%, 99%, or 100% identity to the SEQ ID NO: 210

(MQLELSTCVFLCLLPLGFSAIRRYYLGAVELSWDYRQSELLRELHVDTR
FPATAPGALPLGPSVLYKKTVFVEFTDQLFSVARPRPPWMGLLGPTIQAE
VYDTVVVTLKNMASHPVSLHAVGVSFWKSSEGAEYEDHTSQREKEDDKVL
PGKSQTYVWQVLKENGPTASDPPCLTYSYLSHVDLVKDLNSGLIGALLVC
REGSLTRERTQNLHEFVLLFAVFDEGKSWHSARNDSWTRAMDPAPARAQP
AMHTVNGYVNRSLPGLIGCHKKSVYWHVIGMGTSPEVHSIFLEGHTFLVR
HHRQASLEISPLTFLTAQTFLMDLGQFLLFCHISSHHHGGMEAHVRVESC
AEEPQLRRKADEEEDYDDNLYDSDMDVVRLDGDDVSPFIQIRSVAKKHPK
TWVHYIAAEEEDWDYAPLVLAPDDRSYKSQYLNNGPQRIGRKYKKVRFMA
YTDETFKTREAIQHESGILGPLLYGEVGDTLLIIFKNQASRPYNIYPHGI
TDVRPLYSRRLPKGVKHLKDFPILPGEIFKYKWTVTVEDGPTKSDPRCLT
RYYSSFVNMERDLASGLIGPLLICYKESVDQRGNQIMSDKRNVILFSVFD
ENRSWYLTENIQRFLPNPAGVQLEDPEFQASNIMHSINGYVFDSLQLSVC
LHEVAYWYILSIGAQTDFLSVFFSGYTFKHKMVYEDTLTLFPFSGETVFM
SMENPGLWILGCHNSDFRNRGMTALLKVSSCDKNTGDYYEDSYEDISAYL
LSKNNAIEPRSFAQNSRPPSASAPKPPVLRRHQRDISLPTFQPEEDKMDY
DDIFSTETKGEDFDIYGEDENQDPRSFQKRTRHYFIAAVEQLWDYGMSES
PRALRNRAQNGEVPRFKKVVFREFADGSFTQPSYRGELNKHLGLLGPYIR
AEVEDNIMVTFKNQASRPYSFYSSLISYPDDQEQGAEPRHNFVQPNETRT
YFWKVQHHMAPTEDEFDCKAWAYFSDVDLEKDVHSGLIGPLLICRANTLN
AAHGRQVTVQEFALFFTIFDETKSWYFTENVERNCRAPCHLQMEDPTLKE
NYRFHAINGYVMDTLPGLVMAQNQRIRWYLLSMGSNENIHSIHFSGHVFS
VRKKEEYKMAVYNLYPGVFETVEMLPSKVGIWRIECLIGEHLQAGMSTTF
LVYSKKCQTPLGMASGHIRDFQITASGQYGQWAPKLARLHYSGSINAWST
KEPFSWIKVDLLAPMIIHGIKTQGARQKFSSLYISQFIIMYSLDGKKWQT
YRGNSTGTLMVFFGNVDSSGIKHNIFNPPIIARYIRLHPTHYSIRSTLRM
ELMGCDLNSCSMPLGMESKAISDAQITASSYFTNMFATWSPSKARLHLQG
RSNAWRPQVNNPKEWLQVDFQKTMKVTGVTTQGVKSLLTSMYVKEFLISS
SQDGHQWTLFFQNGKVKVFQGNQDSFTPVVNSLDPPLLTRYLRIHPQSWV
HQIALRMEVLGCEAQDLYV).

β-globin can have or include the following amino acid sequence: SEQ ID NO 211. In various embodiments, a β-globin replacement protein can have an amino acid sequence having at least 70%, 75%, 80%, 85%, 90%, 91%, 92%, 93%, 94%, 95%, 96%, 97%, 98%, 99%, or 100% identity to SEQ ID NO: 211

(MVHLTPEEKSAVTALWGKVNVDEVGGEALGRLLVVYPWTQRFFESFGDL
STPDAVMGNPKVKAHGKKVLGAFSDGLAHLDNLKGTFATLSELHCDKLHV
DPENFRLLGNVLVCVLAHHFGKEFTPPVQAAYQKVVAGVANALAHKYH).

γ-globin can have or include the following amino acid sequence: SEQ ID NO 212. In various embodiments, a γ-globin replacement protein can have an amino acid sequence having at least 70%, 75%, 80%, 85%, 90%, 91%, 92%, 93%, 94%, 95%, 96%, 97%, 98%, 99%, or 100% identity to SEQ ID NO: 212

(MGHFTEEDKATITSLWGKVNVEDAGGETLGRLLVVYPWTQRFFDSFGNL
SSASAIMGNPKVKAHGKKVLTSLGDATKHLDDLKGTFAQLSELHCDKLHV
DPENFKLLGNVLVTVLAIHFGKEFTPEVQASWQKMVTAVASALSSRYH).

In certain exemplary embodiments, a vector of the present disclosure selectively targets a hematopoietic cell type that is or includes T cells and/or a T cell progenitor cell type (e.g., a T cell progenitor cell type that is not a hematopoietic stem cell type, such as CLP cells). In certain exemplary embodiments, a vector of the present disclosure selectively targets a hematopoietic cell type that is or includes T cells and/or a T cell progenitor cell type and encodes a CAR in its genome. As is discussed elsewhere herein, infection and/or transduction of T cells and/or a T cell progenitor cell type by a vector that encodes a CAR in its genome (e.g., for in vivo gene therapy) is associated with certain therapeutic benefits. As compared modification of HSCs to encode and/or express a CAR (e.g., by in vivo gene therapy), modification of a more differentiated cell type (e.g., CLP cells or T cells) to encode and/or express a CAR (e.g., by in vivo gene therapy) shortens the time between modification of the cells and achieving certain, substantial, and/or therapeutically effective numbers or concentrations of T cells expressing the CAR in the subject. In various embodiments, a reduction in time to therapeutic efficacy can be accounted for, at least in part, by bypassing time required for engineered HSCs to produce (or produce a therapeutically effective number or concentration of) T cells expressing the CAR in the subject.

In various embodiments, a vector of the present disclosure that selectively targets a hematopoietic cell type that is or includes T cells and/or a T cell progenitor cell type (e.g., a T cell progenitor cell type that is not a hematopoietic stem cell type, such as CLP cells) can encode a CAR that has a particular target antigen and can be used for treatment of one or more particular types of cancer, e.g., as shown in Table 23 below. In various embodiments, a CAR can target more than one antigen simultaneously, and in various embodiments can be referred to as “dual-targeted” or “combo-targeted” CAR.

TABLE 23
Target Antigens of CARs Useful for Treatment of Particular Types of Cancer

| Target Antigen | Cancer | Further Description |
|---|---|---|
| CD19 | BALL | See clinical trial NCT01044069 |
| CD19 | B-CLL | See clinical trial NCT00466531 |
| CD19 | Leukemia | See clinical trial NCT01416974 |
| CD19 | Lymphoma | See clinical trial NCT00586391 |
| CD19 | B-NHL/CLL | See clinical trial NCT00608270 |
| CD20 | Mantel cell leukemia/B-NHL | See clinical trial NCT00621452 |
| CD22 | Non-Hodgkins Lymphoma | See clinical trial NCT02315612 |
| CD19/CD20 | B-Non Hodgkins Lymphoma | See clinical trial NCT00621452 |
| CD19/CD22 | B-cell malignancy | See clinical trial NCT03185494 |
| CD133 | Hepatocellular carcinoma | See clinical trial NCT02541370 |
| CD171 | Neuroblastoma | See clinical trial NCT02311621 |
| PMSA (prostate-specific membrane | Prostate cancer | See clinical trial NCT001140373 |
| antigen) | | |
| CEA | Breast cancer | See clinical trial NCT00673829 |
| CEA | Colorectal cancer | See clinical trial NCT00673827 |
| CEA | Lungs cancer | See clinical trial NCT00673322 |
| HER-2 | Lungs cancer | See clinical trial NCT00889954 |
| HER-2 | Osteosarcoma | See clinical trial NCT00902044 |
| HER-2 | Glioblastoma | See clinical trial NCT01109095 |
| CD30 | Lymphoma | See clinical trial NCT02274584 |
| FAP | Malignant pleural mesothelioma | See clinical trial NCT01722149 |
| NKGD2 (natural killer group 2 | Leukemia | See clinical trial NCT02203825 |
| member D) | | |
| GD2 (disganglioside molecule) | Neuroblastoma, osteosarcoma | See clinical trial NCT03356795 |
| EGFRVIII (epidermal growth factor | Glioblastoma | See clinical trial NCT02309373 |
| receptor variant III) | | |
| Mesothelin | Pancreatic cancer | See clinical trial NCT02706782 |
| CD38/CD123 | B-Cell Malignancies | See clinical trial NCT03125577 |
| ROR1 (receptor tyrosine kinase- | Chronic lymphocytic leukemia | See clinical trial NCT02194374 |
| like orphan receptor 1) | | |
| MUC16 (CA125) | Ovarian carcinoma | See clinical trial NCT02498912 |
| GPC3 (glypican 3) | Lung squamous cell carcinoma | See clinical trial NCT03198546 |
| VEGFRII (vascular endothelial | Renal cancer | See clinical trial NCT01218867 |
| growth factor receptor II) | | |
| CD 19 and CD 22 | B cell malignancies | One CAR with two binding sites; 4- |
| | | 1BB co-stimulation domain |
| CD 19 and CD 20 | B-NHL | One CAR with two binding sites; 4- |
| | | 1BB co-stimulation domain |
| CD 19 and CD 22 | B-ALL | One vector encoding two CARs; 4- |
| | | 1BB co-stimulation domain for CD |
| | | 19, OX4O co-stimulation domain |
| | | for CD 22 |
| CD 19 and CD 22 | DLBCL | One vector encoding two CARs; 4- |
| | | 1BB co-stimulation domain for CD |
| | | 19, OX4O co-stimulation domain |
| | | for CD 22 |
| CD 19 and CD 22 | B-ALL | One CAR with two binding sites; 4- |
| | | 1BB co-stimulation domain |
| CD 19 and CD 22 | B-ALL | One CAR with two binding sites; 4- |
| | | 1BB co-stimulation domain |
| CD 19 and CD 22 | B-ALL | Two vectors encoding two CAR; 4- |
| | | 1BB co-stimulation domain and |
| | | extra PD-L1 co-stimulation domain |
| | | for CD 22 |
| CD 19 and CD 22 | B-ALL | One CAR with two binding sites; 4- |
| | | 1BB co-stimulation domain |
| CD 19 and CD 22 | B-ALL | Two vectors encoding two CARS; |
| | | 4-1BB co-stimulation domain |
| CD 19 and CD 22 | B-ALL | Manufacture and infuse separately; |
| | | CD 28 and 4-1BB co-stimulation |
| | | domains for both CAR |
| CD 19 and CD 22 | B-NHL | Manufacture and infuse separately; |
| | | CD 28 and 4-1BB co-stimulation |
| | | domains for both CAR |
| EGFR (epidermal growth factor | Lung, liver, stomach | See clinical trials NCT03179007, |
| receptor) | | NCT03525782 (Phase 1/2) |
| HER2 (human epidermal growth | Central nervous system tumor, | See clinical trial NCT03500991 |
| factor receptor 2) | pediatric glioma | (Phase 1) |
| EGFR806 | Central nervous system tumor, | See clinical trial NCT03179012 |
| | pediatric glioma | (Phase 1) |
| Mesothelin | Ovarian, cervical, pancreatic, lung | See clinical trial NCT01583686 |
| | | (Phase 1/2) |
| PSCA (prostate stem cell antigen) | Lung | See clinical trial NCT03198052 |
| | | (Phase 1) |
| MUC1 (mucin1) | Advanced solid tumors, lung | See clinical trials NCT03179007, |
| | | NCT03525782 (Phase 1/2) |
| Claudin 18.2 | Advanced solid tumor | See clinical trial NCT03874897 |
| | | (Phase 1) |
| EpCAM (epithelial cell adhesion | Colon, pancreatic, prostate, gastric, | See clinical trial NCT03013712 |
| molecule) | liver | (Phase 1/2) |
| GD2 | Brain | See clinical trial NCT04099797 |
| | | (Phase 1) |
| VEGFR2 | Melanoma, brain | See clinical trial NCT01218867 |
| | | (Phase 1) |
| AFP (alpha-fetoprotein) | Hepatocellular carcinoma liver | See clinical trial NCT03349255 |
| | cancer | (Phase 1) |
| Nectin4/FAP (familial adenomatous | Nectin4-positive advanced | See clinical trial NCT03932565 |
| polyposis) | malignant solid tumor | (Phase 1) |
| CEA (carcinoembryonic antigen) | Lung, colorectal, gastric, breast, | See clinical trial NCT02349724 |
| | pancreatic cancer | (Phase 1) |
| Lewis Y | Advanced cancer | See clinical trial NCT03851146 |
| | | (Phase 1) |
| Glypican-3 | Liver | See clinical trial NCT02932956 |
| | | (Phase 1) |
| EGFRIII | Glioblastoma and brain tumor | See clinical trial NCT01454596 |
| | | (Phase 1) |
| IL-13Rα2 | Glioblastoma | See clinical trial NCT02208362 |
| | | (Phase 1) |
| CD171 | Neuroblastoma | See clinical trial NCT02311621 |
| | | (Phase 1) |
| MUC16 (mucin16) | Ovarian | See clinical trial NCT02311621 |
| | | (Phase 1) |
| PSMA (prostate-specific membrane | Prostate | See clinical trial NCT01140373 |
| antigen) | | (Phase 1) |
| AFP | Hepatocellular carcinoma, liver | See clinical trial NCT03349255 |
| | | (Phase 1) |
| AXL (AXL receptor tyrosine | Renal | See clinical trial NCT03393936 |
| kinase) | | (Phase 1) |
| CD20 | Melanoma | See clinical trial NCT03893019 |
| | | (Phase 1) |
| CD80/86 | Lung | See clinical trial NCT03198052 |
| | | (Phase 1) |
| c-MET | Breast, hepatocellular | See clinical trials NCT03060356, |
| | | NCT03638206 (Phase 1) |
| DLL-3 (delta-like 3) | Lung | See clinical trial NCT03392064 |
| | | (Phase 1) |
| DR5 (death receptor 5) | Hepatoma | See clinical trial NCT03638206 |
| | | (Phase 1) |
| EpHA2 (EPH receptor A2) | Glioma | See clinical trial NCT02575261 |
| | | (Phase 1) |
| FR-α (folate receptor alpha) | Ovarian | See clinical trial NCT00019136 |
| | | (Phase 1) |
| gp100 | Melanoma | See clinical trial NCT03649529 |
| | | (Phase 1) |
| MAGE (melanoma antigen gene | Lung | See clinical trials NCT03356808, |
| protein)-A1/3/4 | | NCT03535246 (Phase 1) |
| LMP1 (Epstein-Barr virus latent | Nasopharyngeal | See clinical trial NCT02980315 |
| membrane protein 1) | | (Phase 1) |

In certain embodiments, methods and compositions of the present disclosure can be used for treating a human, primate, non-human primate, or mammal. In various embodiments, methods and compositions of the present disclosure can be used for treating an animal. In various embodiments, methods and compositions of the present disclosure can be used for treating an animal such as a dog, cat, bird, chicken, reptile, horse, cow, pig, goat, mouse, rodent, or rat.

### EXAMPLES

The present Examples demonstrate that certain adenoviral serotypes are particularly effective for infection of hematopoietic cells (e.g., one or more particular types of hematopoietic cells). Because hematopoietic cells (e.g., one or more particular types of hematopoietic cells) are a therapeutically important target for gene therapy, identification of vectors effective for their transduction is of substantial clinical importance. The present Examples illustrate certain clinically relevant applications of engineered adenoviral vectors that selective target hematopoietic cells (e.g., one or more particular types of hematopoietic cells).

### Example 1: Analysis of Adenoviral Vector Infection of CD34+ Cells by Anti-Hexon Staining

Preset Examples 1 and 2 demonstrate that certain adenoviral serotypes are particularly effective for infection of CD34+ cells such as HSCs. Because HSCs are a therapeutically important target for gene therapy, identification of vectors effective for transduction of CD34+ cells is of substantial clinical importance. Certain tested adenoviral serotypes were similarly or more effective for infection of CD34+ cells than others commonly associated with gene therapy trials and research, such as Ad5 and Ad5/35++.

The present example utilizes anti-hexon staining to measure the infection of CD34+ cells by various adenoviral vectors. Serotypes used in experiments of this Example included Ad3, Ad5, Ad7, Ad11, Ad14, Ad16, Ad21, Ad26, Ad34, Ad35, Ad37, Ad48, Ad50, and Ad52, as well as an Ad5/35++ vector including E1 deletion (“F35”). Vectors were wild type human adenoviral vectors except as otherwise noted.

Human CD34+ cells (REF: 4Y-101C, LOT: 3038009, Donor ID: 15846) were infected with wild type human adenoviruses (identified by Ad type number) with 5,000 or 2,000 viral particles per cell (vp/c). Three hours post-incubation, cells were first washed with phosphate buffered saline (PBS), quickly trypsinized to remove all extracellular viral particles, and washed with PBS. Washed cells were then split into two aliquots utilized in the present Example for analysis of intra-cellular adenovirus particles by anti-hexon staining and in Example 2 for analysis of adenoviral DNA internalization by qPCR, respectively. A replicate trial was additionally conducted in which CD34+ cells were infected at 2,000, 10,000, and 20,000 viral particles per cell (vp/c).

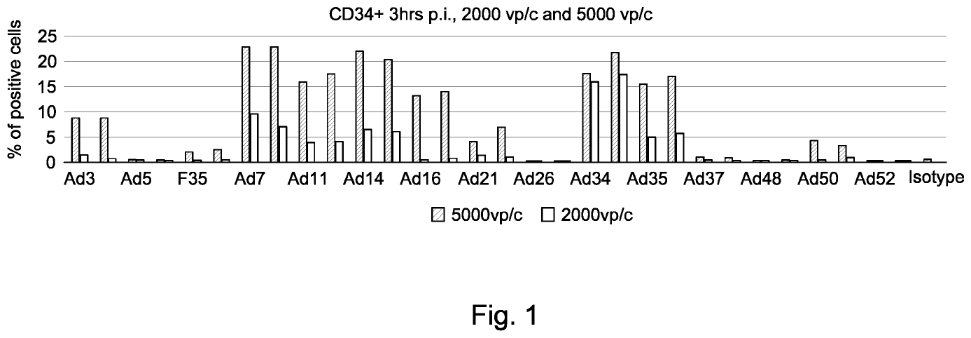

In the present Example, cells were first fixed with fixation medium (Thermofisher) for 15 minutes at room temperature. After a PBS washing step, cells were resuspended in permeabilization medium (Thermofisher). Anti-adenovirus hexon antibody (clone 20/11, MAB8052, Sigma) was added to the permeabilization medium and incubated at 4° C. overnight. On the second day, cells were washed twice with PBS and stained with the Alexa Fluor 488-labeled secondary antibody (Catalog #A-21121, Thermofisher) in permeabilization medium. Staining was stopped with two PBS washing steps, and the cells were analyzed on a Beckman Coulter Gallios Flow Cytometer. Background signal was obtained by analyzing the isotype control, which refers to uninfected cells stained with the same antibodies as the sample. The percentage of FITC positive cells is displayed in the FIG. 1. For each virus two samples are shown for each virus dose.

Results of anti-hexon staining are provided in FIG. 1. Reference serotypes in this Example, as shown in FIG. 1, include Ad5 and Ad5/35++(F35) serotypes that are often used, e.g., that have been used in gene therapy research or adenoviral vector constructs. Unexpectedly, several adenoviral vector serotypes consistently outperformed these reference serotypes for internalization into CD34+ cells. These included Ad3, 7, 11, 14, 16, 21, 34, 35, and 50. By contrast, serotypes Ad26, Ad37, Ad48, and Ad52 consistently did not outperform reference serotypes for internalization into CD34+ cells. These data demonstrate that Ad3, 7, 11, 14, 16, 21, 34, 35, and 50 are particularly and unexpectedly useful for engineering of vectors for transduction of CD34+ cells such as HSCs.

### Example 2: Analysis of the Internalization of Adenovirus Particles into CD34+ Cells by qPCR

The present example utilizes qPCR to measure the internalization of adenovirus particles into CD34+ cells by various adenoviral serotypes. Serotypes used in experiments of this Example included Ad3, Ad5, Ad7, Ad1, Ad14, Ad16, Ad21, Ad26, Ad34, Ad37, Ad35, Ad48, Ad50, and Ad52, as well as Ad5/35++ vector including an E1 deletion (“F35”). The viruses used were purified wild type human adenoviruses except as otherwise noted. Cells were prepared as described in Example 1.

In the present Example, total genomic DNA was isolated using the Monarch® Genomic DNA Purification Kit (NEB). For qPCR analyses, samples were split into two experiments: Ad3, 7, 11, 14, 16, 21, 34, 35, and 50 in a first experiment; and Ad26, Ad37, Ad48, Ad52, Ad5, and F35 in a second experiment. For the first experiment, primers and probe targeting DNA polymerase were used for amplification and a plasmid containing the Ad35 genome (pAd35) was used to generate a standard curve. For the second experiment, primers and probe targeting hexon were used for amplification and a plasmid containing the Ad5 genome (pAd5) was used to generate a standard curve. For normalization, primers that amplify the gene hB2M were applied.

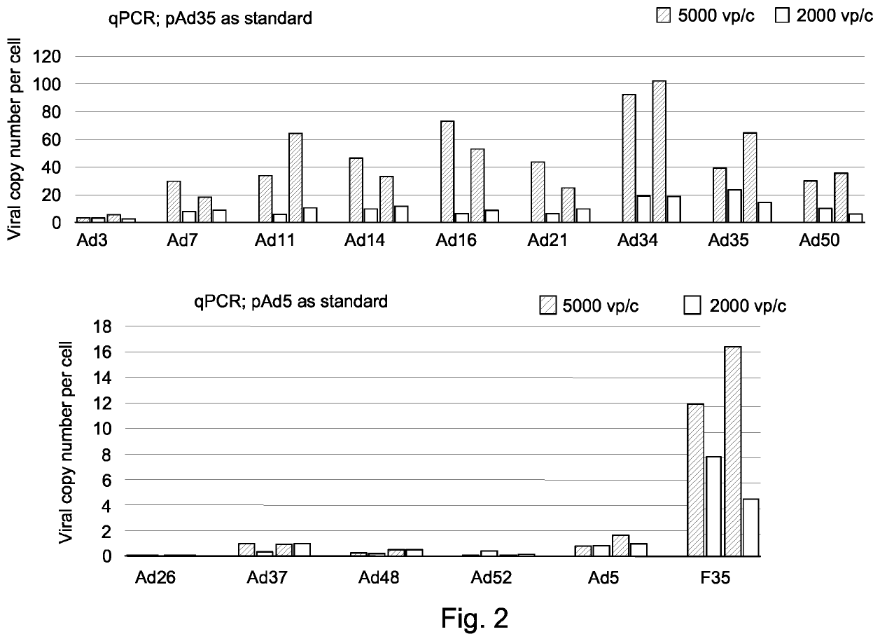

Results of the qPCR analyses of this Example are provided in FIG. 2. Broadly, viral copy number per cell was highest using Ad7, Ad11, Ad14, Ad16, Ad21, Ad34, Ad35, Ad50, and F35. Viral copies per cell were also detected for Ad3, Ad37, Ad48, Ad52, and Ad5. Viral copy number per cell was lowest for Ad26.

### Example 3: Identification of Adenoviral Vectors that Selectively Target a Particular Hematopoietic Cell Type

The present Example provides approaches for identifying adenoviral vectors that selectively target particular hematopoietic cell types. In various embodiments, an anti-hexon staining method, a qPCR method, and/or a fluorescent protein expression-based method can be applied to compare preferential targeting of a hematopoietic cell type of interest as compared to a reference hematopoietic cell type. A cell type of interest can be, for example, T cells (e.g., CD8+ and/or CD4+ T cells), B cell plasmablasts, B cells (e.g., memory B cells), NK cells, or progenitor cells (e.g., CLPs). A reference can be any type or types of hematopoietic stem cells that are not the target of interest, including without limitation HSCs. Data can further be analyzed by comparison to a negative control, e.g., uninfected cells.

Approaches described in this Example can be carried out using various types of samples Approaches described in this Example can be carried out by infection of cell samples that represent isolated samples of one or more particular cell types (e.g., an isolated target cell type and/or an isolated reference cell type). Approaches described in this Example can be carried out by infection of cell samples that represent samples that include a mixture of one or more hematopoietic cell types (including one or more of a target cell type and/or a reference cell type). In various embodiments, cells of one or more particular types (e.g., a target cell type and/or a reference cell type) can be distinguished before or after assaying infection according to one or more approaches set forth in this Example. Methods of distinguishing hematopoietic cell types are known in the art and include the evaluation of cell type markers.

Hexon staining includes infecting target and reference cells with one or more Ad vectors. Cells are assayed three hours post-incubation. The assay includes washing cells with phosphate buffered saline (PBS), quickly trypsinizing cells to remove all extracellular viral particles, and washing again with PBS. Cells are then fixed with fixation medium (Thermofisher) for 15 minutes at room temperature. After a PBS washing step, cells are resuspended in permeabilization medium (Thermofisher). Anti-adenovirus hexon antibody (clone 20/11, MAB8052, Sigma) is then added to the permeabilization medium and samples are incubated at 4° C. overnight. On the second day, cells are washed twice with PBS and stained with the Alexa Fluor 488-labeled secondary antibody (Catalog #A-21121, Thermofisher) in permeabilization medium. Staining is stopped with two PBS washing steps, and cells are analyzed on a Beckman Coulter Gallios Flow Cytometer. Background signal is obtained by analyzing the negative control, which refers to uninfected cells stained with the same antibodies as the sample. The percentage of FITC positive cells is determined.

qPCR analysis includes infecting target and reference cells with one or more Ad vectors. Cells are assayed three hours post-incubation. The assay includes washing cells with phosphate buffered saline (PBS), quickly trypsinizing cells to remove all extracellular viral particles, and washing again with PBS. Total genomic DNA is then isolated from cells. Primers and probes targeting one or more vector genome sequences (e.g., a hexon-encoding sequence) can be used for qPCR. For normalization, primers and probes targeting a housekeeping gene can be used.

Fluorescent protein expression-based analysis includes infecting target and reference cells with one or more Ad vectors that include an Ad genome encoding a fluorescent protein such as a Green Fluorescent Protein (GFP) or mCherry. The fluorescent protein is encoded by an integrating element of the Ad genome, and is expressed after integration. Accordingly, detection of fluorescence by each cell type tested is indicative of transduction.

### Example 4: Infection of T-Cells by Adenoviral Vectors Encoding a Chimeric Antigen Receptor

The present Example includes identification of an adenoviral vector that selectively targets CD8+ T cells according to one or more approaches set forth in Example 3, and use thereof to introduce into CD8+ T cells a nucleic acid payload encoding a CAR. Without limiting the disclosure, exemplary adenoviral vectors that selectively target CD8+ T cells can include adenoviral vectors of serotypes Ad5, Ad16, Ad34, Ad35, and Ad35++. Selected adenoviral vectors are engineered to include an adenoviral vector genome that includes a payload that encodes a CAR. The nucleic acid sequence encoding the CAR is an integrating nucleic acid sequence. Engineered adenoviral vectors including the nucleic acid sequence encoding the CAR, in combination where applicable with a support vector of the same serotype (e.g., single or chimeric serotype), are administered to a subject or system such as a human patient in need thereof. The nucleic acid sequence encoding the CAR is integrated into the genome of CD8+ T cells of the recipient subject or system, providing a therapeutic benefit where applicable. Certain produced cells can be referred to as CAR-T cells. Gene therapy according to this example can be characterized by therapeutically advantageous properties including immediacy of effect and transiency of effect.

### Example 5: Infection of NK Cells by Adenoviral Vectors Encoding a Chimeric Antigen Receptor

The present Example includes identification of an adenoviral vector that selectively targets NK cells according to one or more approaches set forth in Example 3, and use thereof to introduce into NK cells a nucleic acid payload encoding a CAR. Without limiting the disclosure, exemplary adenoviral vectors that selectively target NK cells can include adenoviral vectors of serotypes Ad11, Ad16, Ad34, Ad35, and Ad35++. Selected adenoviral vectors are engineered to include an adenoviral vector genome that includes a payload that encodes a CAR. The nucleic acid sequence encoding the CAR is an integrating nucleic acid sequence. Engineered adenoviral vectors including the nucleic acid sequence encoding the CAR, in combination where applicable with a support vector of the same serotype (e.g., single or chimeric serotype), are administered to a subject or system such as a human patient in need thereof. The nucleic acid sequence encoding the CAR is integrated into the genome of NK cells of the recipient subject or system, providing a therapeutic benefit where applicable. Certain produced cells can be referred to as CAR-NK cells. Gene therapy according to this example can be characterized by therapeutically advantageous properties including immediacy of effect and transiency of effect.

### Example 6: Infection of Monocytes by Adenoviral Vectors Encoding a Chimeric Antigen Receptor

The present Example includes identification of an adenoviral vector that selectively targets monocytes according to one or more approaches set forth in Example 3, and use thereof to introduce into monocytes a nucleic acid payload encoding a CAR. Without limiting the disclosure, exemplary adenoviral vectors that selectively target monocytes cells can include adenoviral vectors of serotypes Ad11, Ad16, Ad34, Ad35, and Ad35++. Selected adenoviral vectors are engineered to include an adenoviral vector genome that includes a payload that encodes a CAR. The nucleic acid sequence encoding the CAR is an integrating nucleic acid sequence. Engineered adenoviral vectors including the nucleic acid sequence encoding the CAR, in combination where applicable with a support vector of the same serotype (e.g., single or chimeric serotype), are administered to a subject or system such as a human patient in need thereof. The nucleic acid sequence encoding the CAR is integrated into the genome of monocytes of the recipient subject or system, providing a therapeutic benefit where applicable. Certain produced cells can be referred to as CAR-M cells. Gene therapy according to this example can be characterized by therapeutically advantageous properties including immediacy of effect and transiency of effect.

### Example 7: Infection of Progenitor Cells by Adenoviral Vectors Encoding a Chimeric Antigen Receptor

The present Example includes identification of an adenoviral vector that selectively targets progenitor cells such as CLP cells according to one or more approaches set forth in Example 3, and use thereof to introduce into progenitor cells such as CLP cells a nucleic acid payload encoding a CAR. Selected adenoviral vectors are engineered to include an adenoviral vector genome that includes a payload that encodes a CAR. The nucleic acid sequence encoding the CAR is an integrating nucleic acid sequence. Engineered adenoviral vectors including the nucleic acid sequence encoding the CAR, in combination where applicable with a support vector of the same serotype (e.g., single or chimeric serotype), are administered to a subject or system such as a human patient in need thereof. The nucleic acid sequence encoding the CAR is integrated into the genome of progenitor cells such as CLP cells of the recipient subject or system, providing a therapeutic benefit where applicable. Gene therapy according to this example can be characterized by therapeutically advantageous properties including expression of CAR by multiple lineages including production of CAR-T cells and engineered B cells.

### Example 8: Infection of B Cell Plasmablasts by Adenoviral Vectors Encoding an Antibody

The present Example includes identification of an adenoviral vector that selectively targets B cell plasmablasts according to one or more approaches set forth in Example 3, and use thereof to introduce into B cell plasmablasts a nucleic acid payload encoding an antibody. Selected adenoviral vectors are engineered to include an adenoviral vector genome that includes a payload that encodes an antibody. The nucleic acid sequence encoding the antibody is an integrating nucleic acid sequence. Engineered adenoviral vectors including the nucleic acid sequence encoding the antibody, in combination where applicable with a support vector of the same serotype (e.g., single or chimeric serotype), are administered to a subject or system such as a human patient in need thereof. The nucleic acid sequence encoding the antibody is integrated into the genome of B cell plasmablasts of the recipient subject or system, providing a therapeutic benefit where applicable. B cell plasmablasts are understood to be short-lived, but efficient for antibody secretion. Gene therapy according to this example can be characterized by therapeutically advantageous properties including immediacy of effect and transiency of effect.

### Example 9: Infection of Memory B Cells by Adenoviral Vectors Encoding an Antibody

The present Example includes identification of an adenoviral vector that selectively targets memory B cells according to one or more approaches set forth in Example 3, and use thereof to introduce into memory B cells a nucleic acid payload encoding an antibody. Without limiting the disclosure, exemplary adenoviral vectors that selectively target memory B cells can include adenoviral vectors of serotype Ad16. Selected adenoviral vectors are engineered to include an adenoviral vector genome that includes a payload that encodes an antibody. The nucleic acid sequence encoding the antibody is an integrating nucleic acid sequence. Engineered adenoviral vectors including the nucleic acid sequence encoding the antibody, in combination where applicable with a support vector of the same serotype (e.g., single or chimeric serotype), are administered to a subject or system such as a human patient in need thereof. The nucleic acid sequence encoding the antibody is integrated into the genome of memory B cells of the recipient subject or system, providing a therapeutic benefit where applicable. Memory B cells are understood to produce and/or constitute a quiescent pool that can yield activated plasma B cells under inducing conditions. Gene therapy according to this example can be characterized by therapeutically advantageous properties including long-term potential efficacy.

### Example 10: Identification of Adenoviral Vectors that Selectively Target Monocytes, T Cells, NK Cells, and B Cells

The present Example demonstrates the identification of certain adenoviral serotypes that are particularly effective for infection of particular hematopoietic cell types (e.g., monocytes, T cells, NK cells, and B cells). First generation adenoviral vectors of various adenoviral serotypes encoding a green fluorescent protein (GFP) reporter gene were used to transduce cells of various cell types to determine the adenoviral serotypes that are particularly effective for each cell type. Serotypes tested in the present Example include Ad5, Ad7, Ad11, Ad16, Ad34, and Ad35, as well as an Ad35 vector with an Ad35++ fiber knob (“Ad35++”).

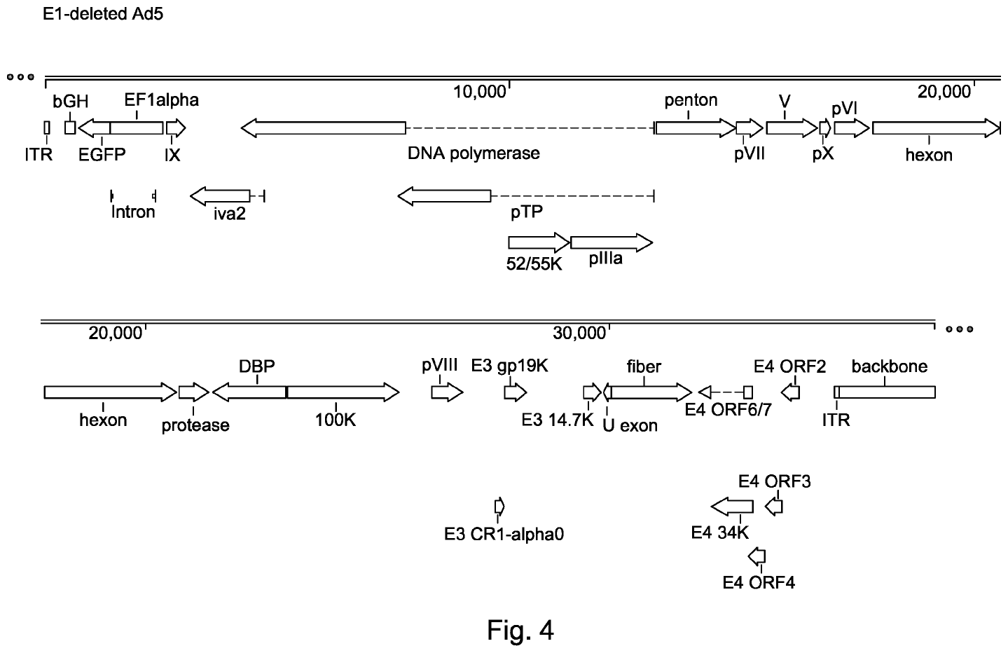

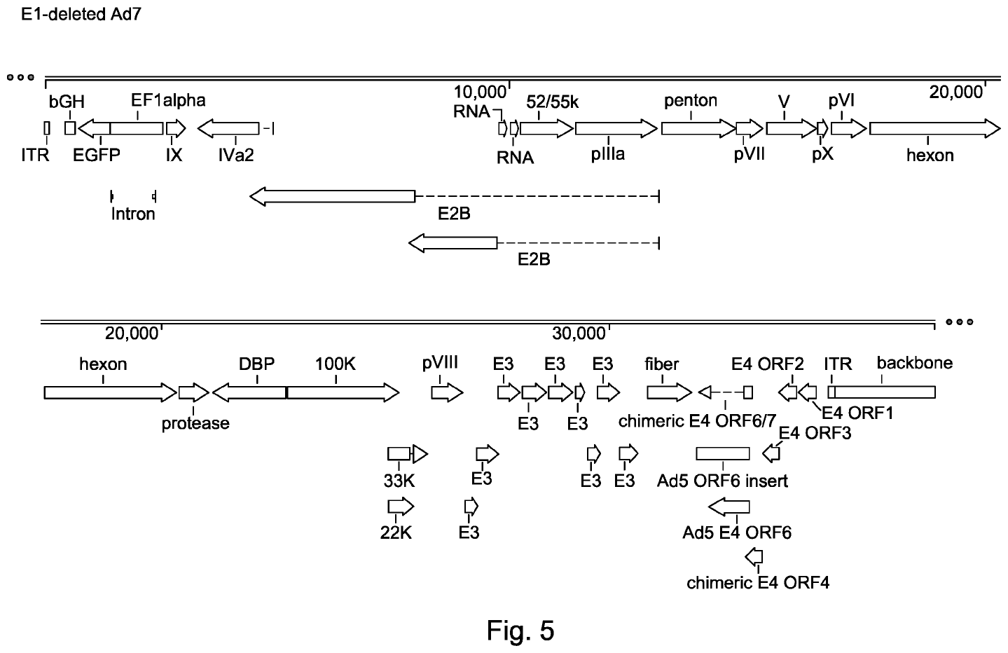

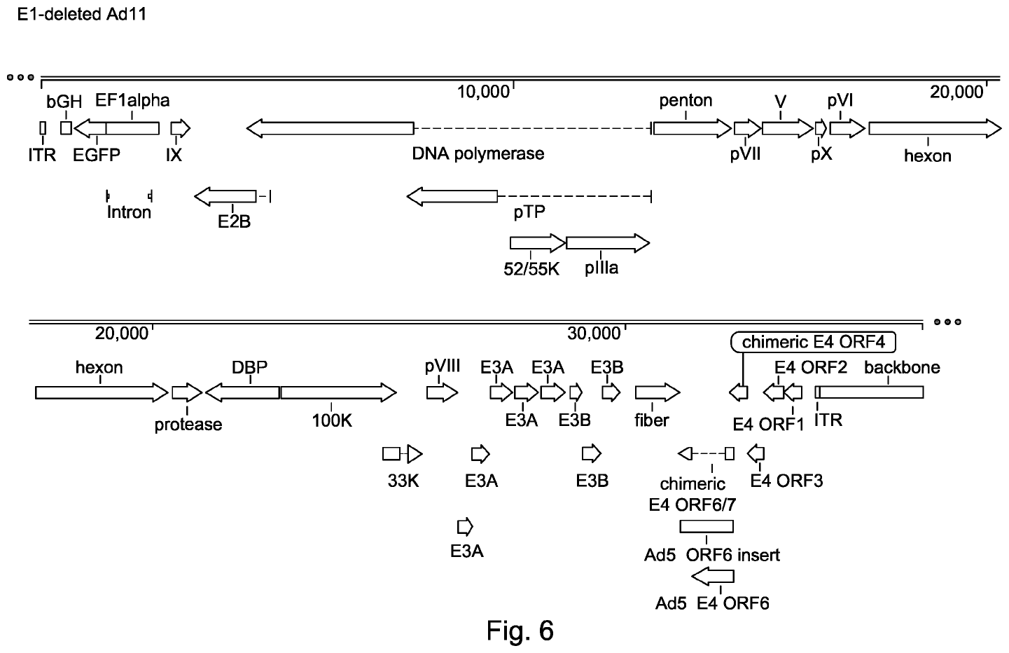

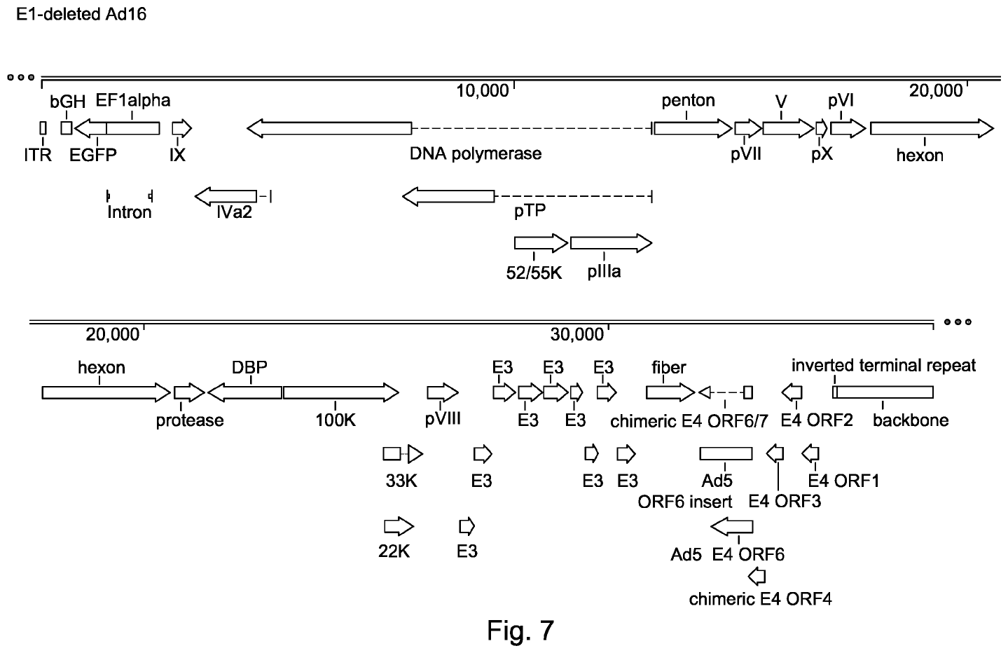

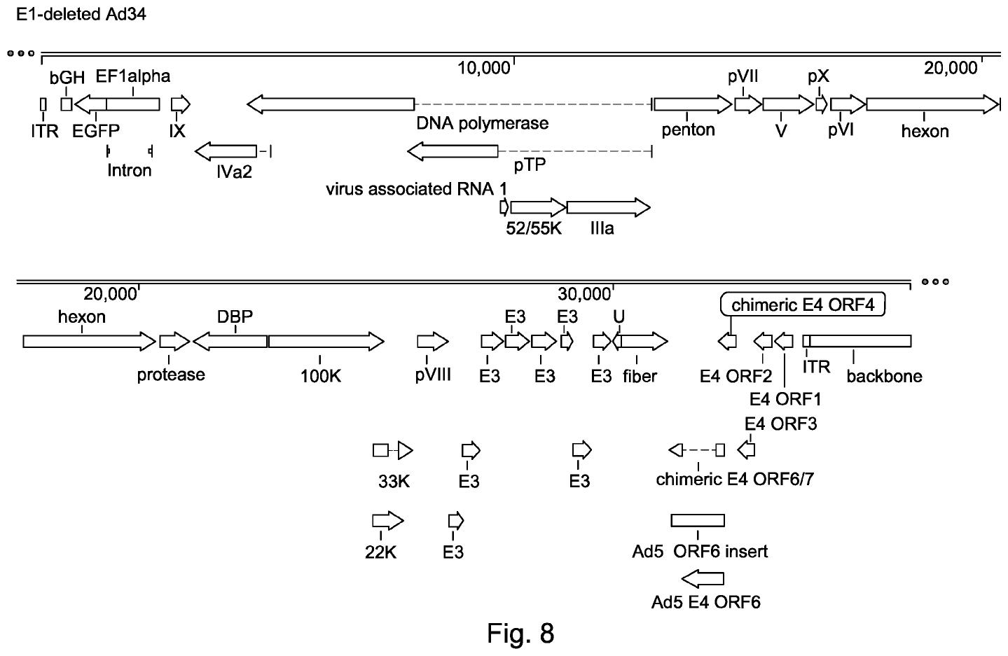

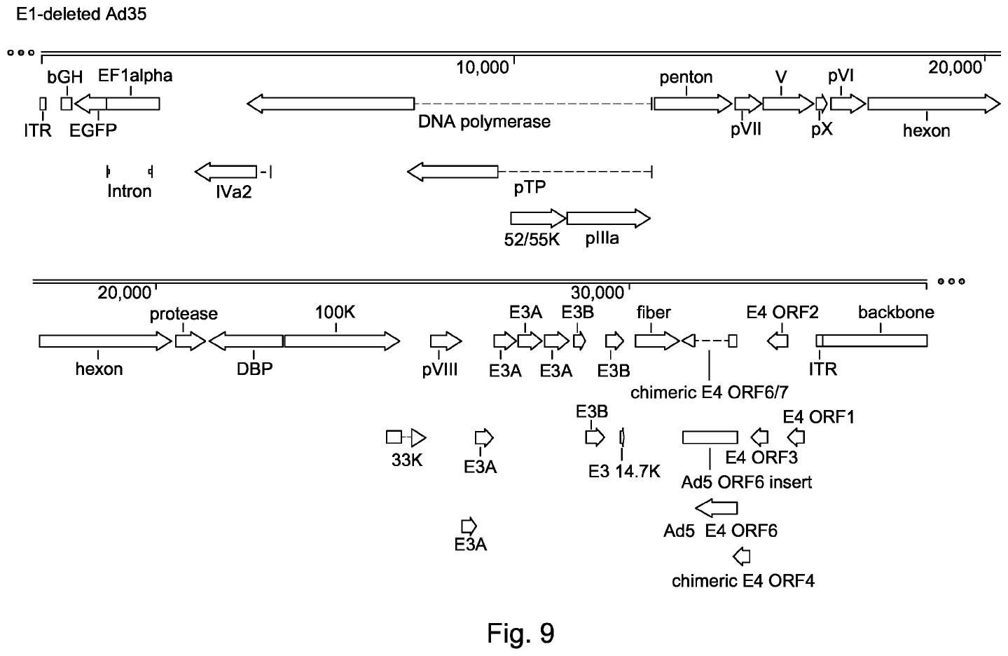

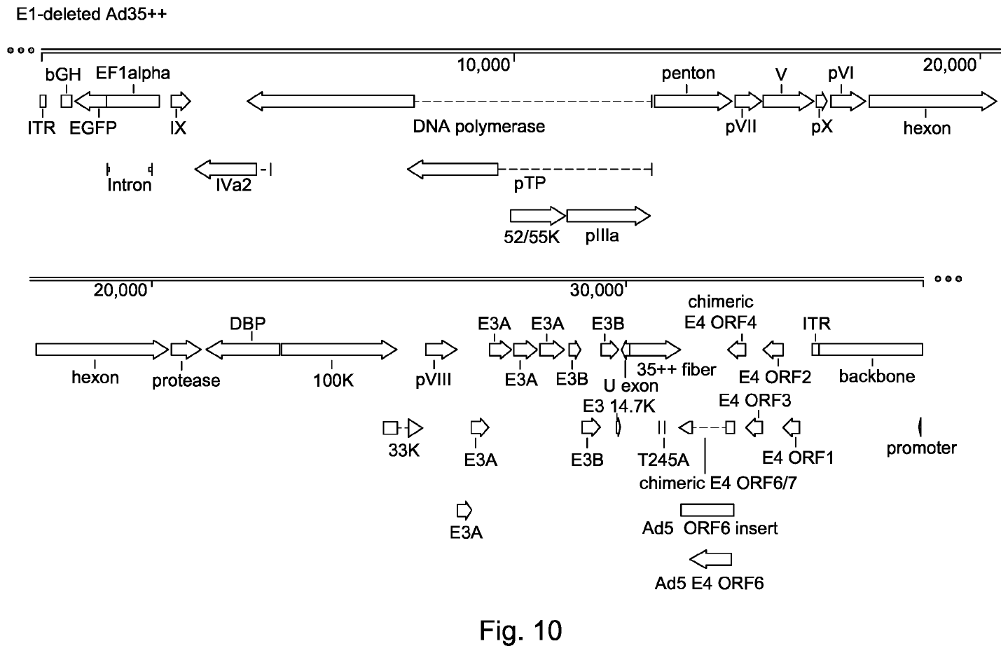

First generation adenoviral genomes were generated from wild-type A5, Ad7, Ad11, Ad16, Ad34, and Ad35 genomes. Relative to the wild-type adenoviral genomes, first generation adenoviral genomes were engineered to have the E1 region (E1a and E1b) deleted and replaced with a GFP reporter gene under the control of an EF1α promoter and a bovine growth hormone (BGH) polyadenylation signal. First generation A7, Ad11, Ad16, Ad34, Ad35, and Ad35++ genomes additionally included deletion of the endogenous E4orf6 region and replacement with an Ad5 E4orf6 regions to facilitate propagation of the first-generation adenoviral vectors in HEK293 cells. Those of skill in the art will understand that replacement of the endogenous E4orf6 regions with an Ad5 E4orf6 region is not required for generation of adenoviral vectors of the present disclosure. Furthermore, first generation Ad35++ genomes included a mutant Ad35++ fiber knob, as described elsewhere herein. Schematics of plasmids encoding the first generation adenoviral genomes are shown in FIGS. 4-11.

Plasmids encoding the first generation adenoviral genomes were digested using restriction enzymes to release the adenoviral genomes, which were subsequently purified. To produce first generation adenoviral vectors, HEK293 cells seeded in 6 cm dishes were transfected with 4 μg of the purified adenoviral genomic DNA using OPTIMUS transfection reagent (polyPlus, 101000006), according to manufacturer's suggested protocol. Cell culture media was replaced 4 hours after transfection or the following day. Once cytopathic effect (CPE) was observed (typically 2-5 days after transfection), the cell-virus lysate was collected and used for serial vector amplification in HEK293 cells using a third of the lysate from the previous step. Large-scale vector production was performed using 20-30 dishes (15 cm) of HEK293 cells infected at 95% confluency. After two days and visible CPE, the cells were harvested. First generation adenoviral vectors were purified using CsCl gradients and ultracentrifugation, as previously described in Jager and Ehrhardt, Hum Gene Ther, 20(8):883-896 (2009), followed by dialysis. The purified viral titer was determined by measuring the optical density using the formula: optical units per mL (OPU)=(absorbance at 260 nm)×(dilution factor)×(1.1×10¹²)×36/(size of adenoviral genome in kb). Successful production of first generation adenoviral vectors was confirmed by restriction enzyme digestion of adenoviral genomic DNA isolated from the adenoviral vectors.

To identify the adenoviral serotypes that are particularly effective for infection of particular hematopoietic cell types, human peripheral blood mononuclear cells (PBMCs) from two donors (Donor 1 and Donor 2) were infected with the purified first generation adenoviral vectors. First generation Ad16 vector was not used in experiments using PBMCs from Donor 2. For infection, 600,000 cells were infected in a volume of less than 200 μl of culture media (RPMI with 10% fetal bovine serum) in ultralow attachment plates. Cells were infected at a multiplicity of infection (MOI) of 500, 2000, and 5000 viral particles per cell. Culture media was changed three hours post-infection.

Flow cytometry was used to analyze the PBMCs 48 hours post-infection. The PBMCs were wash with 0.5% BSA in PBS and resuspended in blocking buffer (47 μl Brilliant Stain Buffer (BD Biosciences) with 3 μl human TruStain FcX (BioLegend)) at 4° C. for 15 minutes. To distinguish various hematopoietic cell types present in the PBMCs, the cells were separately stained using each of two cocktails of antibodies (Tables 24 and 25). The antibodies were separated into two cocktails to avoid spectral overlap. 50 μl of the antibody cocktails were incubated with the samples 4° C. for 20 minutes, followed by a wash using 0.5% BSA in PBS. For Donor 1, samples were incubated in 5% 7AAD in Brilliant Stain Buffer at 4° C. for 20 minutes for live cell discrimination. Samples were analyzed using a CytoFLEX flow cytometer (Beckman Coulter). Infected cells could be identified by detecting GFP payload expression. Each sample was collected in technical duplicate.

TABLE 24
Antibody Cocktail 1

| Component | Format | Amount |
|---|---|---|
| anti-CD45 | BV605/V610 | 2 μl |
| anti-CD3 | BV421/PB450 | 2 μl |
| anti-CD56 | PE-Cy7/PC7 | 2 μl |
| Buffer | Brilliant Stain Buffer | 44 μl |

TABLE 25
Antibody Cocktail 2

| Component | Format | Amount |
|---|---|---|
| anti-CD45 | BV605/V610 | 2 μl |
| anti-CD20 | APC | 2 μl |
| anti-CD14 | BV421/PB450 | 2 μl |
| anti-CD11 | PE-Cy7/PC7 | 1 μl |
| Buffer | Brilliant Stain Buffer | 43 μl |

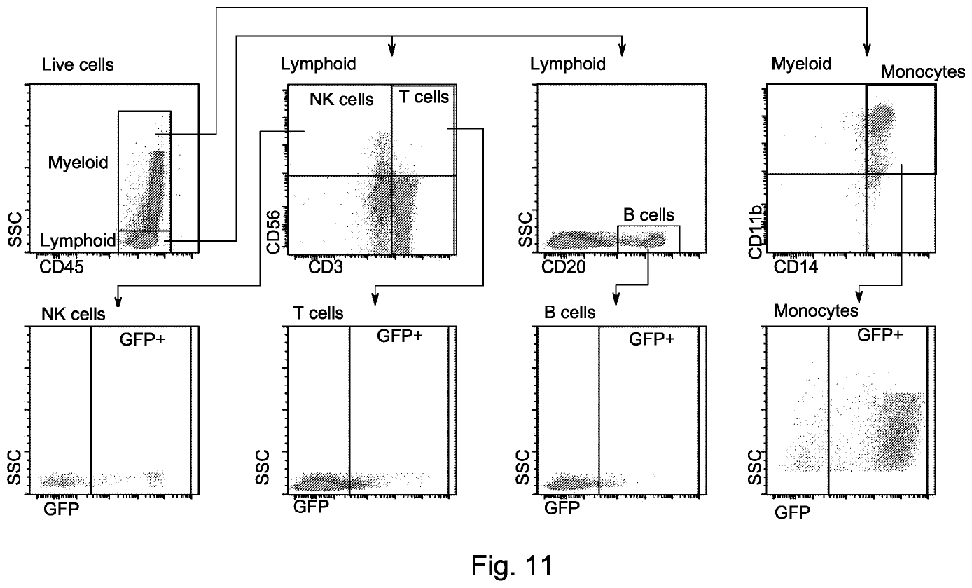

The flow cytometry data was analyzed to identify particular hematopoietic cell types present in the PBMCs. From 7AAD negative cells (live cells) (Donor 1) or all cells (Donor 2), CD45+ leukocytes were identified. The leukocyte population was separated into lymphoid and myeloid cell populations based on forward scatter (FSC) and side scatter (SSC). From the myeloid population, CD11+/CD14+ monocytes were identified. From the lymphoid population, CD3+ T cells, CD3−/CD56+NK cells, and CD20+ B cells were identified. Within each cell type population (i.e., monocytes, T cells, NK, cells, and B cells), the percentage of GFP positive cells was quantified and used to determine the adenoviral serotypes that are particularly effective for infection the cell type. An exemplary gating strategy is shown in FIG. 11.

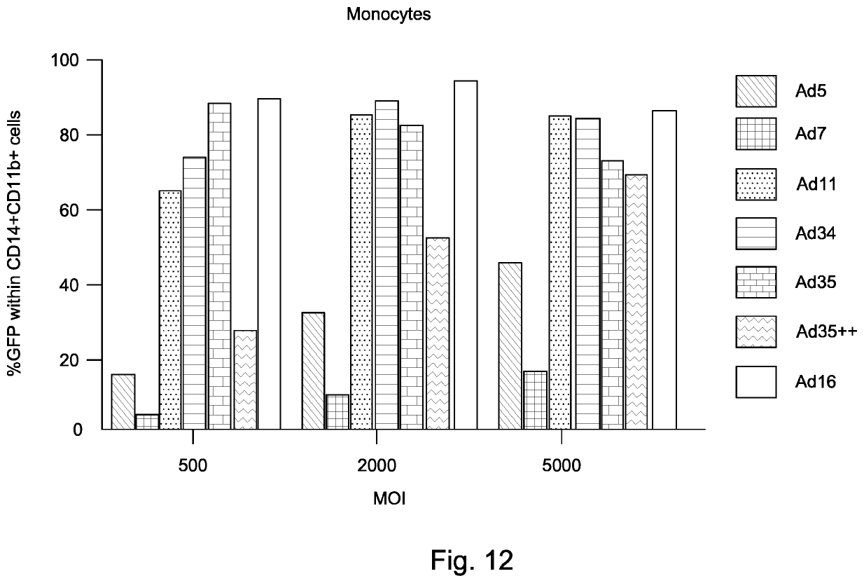

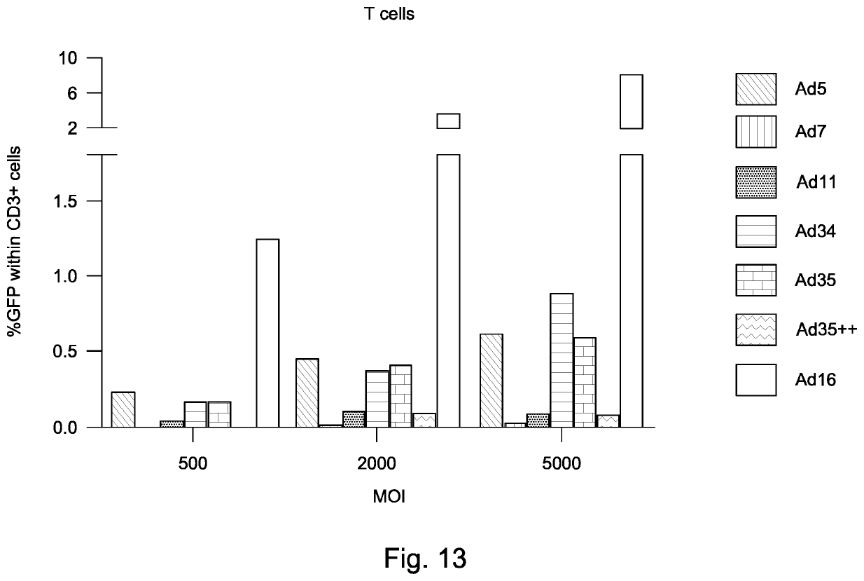

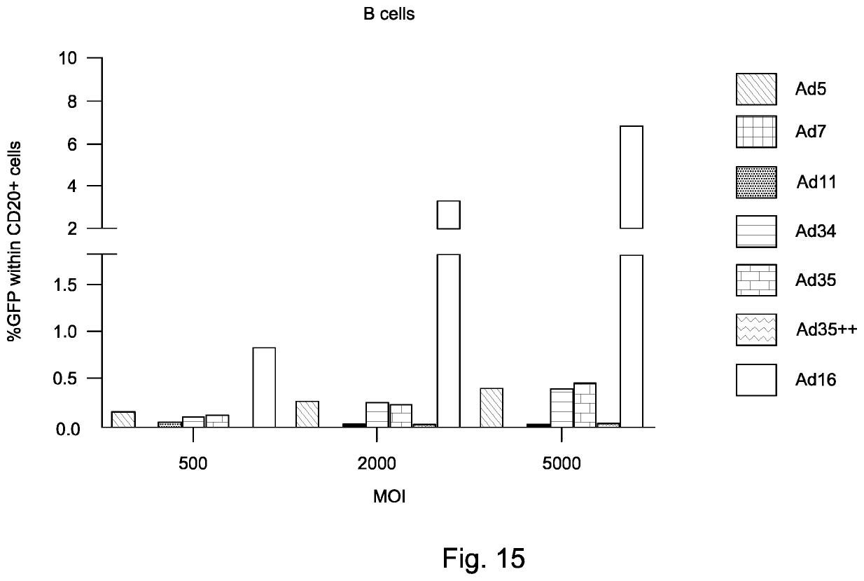

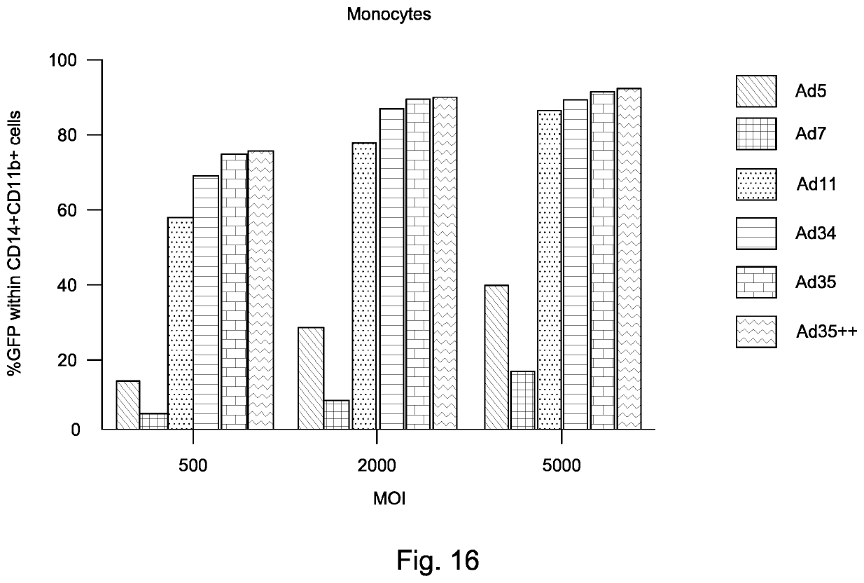

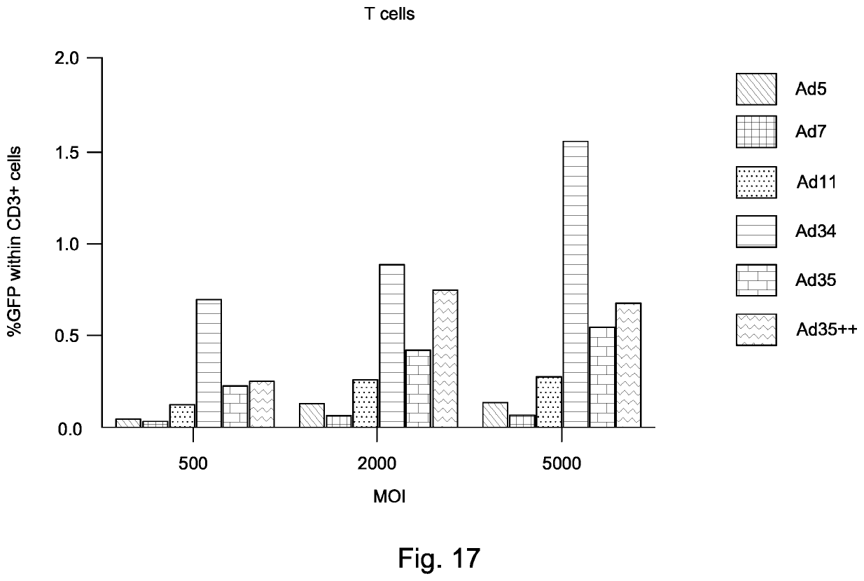

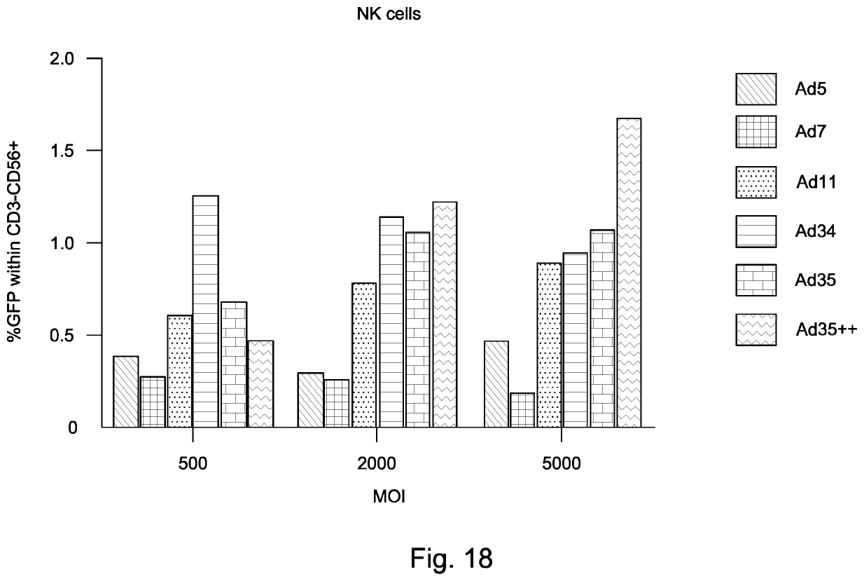

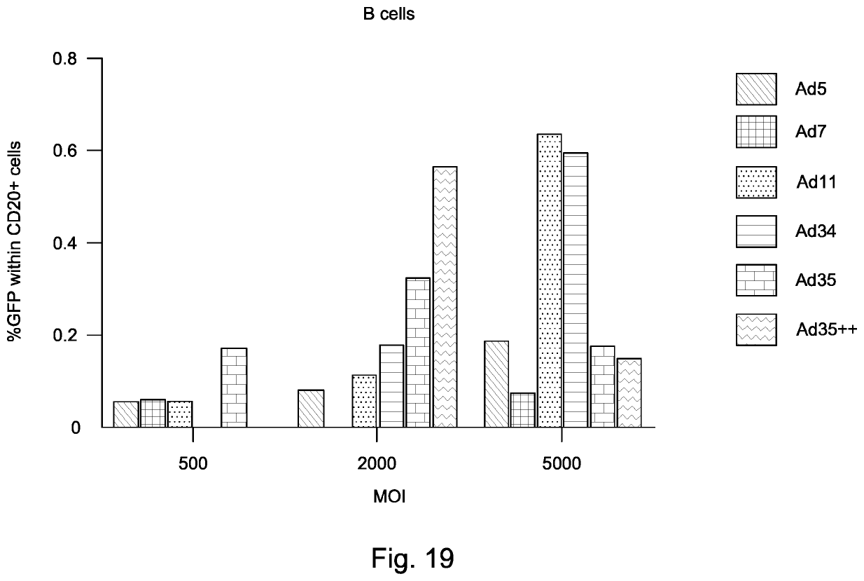

Results for each of the cell types are shown for Donor 1 in FIGS. 12-15 and for Donor 2 in FIGS. 16-19. Donor 1 monocytes were preferentially infected by first generation Ad11, Ad16, Ad34, Ad35, and Ad35++ vectors (FIG. 12). Donor 2 monocytes were preferentially infected by first generation Ad11, Ad34, Ad35, and Ad35++ vectors (FIG. 16). Monocytes showed higher infection rate (percentage of GFP positive) as compared to the lymphoid cells (i.e., T cells, NK cells, and B cells), which, without wishing to be bound by any particular theory, may be due to the phagocytic activity of monocytes. Donor 1 T cells were preferentially infected by first generation Ad5, Ad16, Ad34, and Ad35 vectors (FIG. 13). Donor 2 T cells were preferentially infected by first generation Ad34, Ad35, Ad35++(FIG. 17). Donor 1 NK cells were preferentially infected by first generation Ad1, Ad16, Ad34, Ad35, and Ad35++ vectors (FIG. 14). Donor 2 NK cells were preferentially infected by first generation Ad11, Ad34, Ad35, and Ad35++ vectors (FIG. 18). Donor 1 B cells were preferentially infected by first generation Ad16 vectors (FIG. 15). These data demonstrate that serotypes Ad5, Ad11, Ad16, Ad34, Ad35, and Ad35++ can be engineered into vectors for transduction of particular hematopoietic cell types.

### Accession Sequences

Provided herein is a listing of nucleic acid sequences and amino acid sequences corresponding to publicly available sequence accession numbers, certain of which sequences and/or sequence accession numbers are included and/or utilized, in whole and/or in part, in the present disclosure, and/or certain of which sequences and/or sequence accession numbers are included herein as references. Sequences associated with accession numbers are available in publicly accessible databases, as is known to those of skill in the art, and such sequences are provided herein solely for easy for reference.

GenBank Accession No. AP_000601 (SEQ ID NO: 256)

MTKRVRLSDSFNPVYPYEDESTSQHPFINPGFISPNGFTQSPDGVLTLKC
LTPLTTTGGSLQLKVGGGLTVDDTDGTLQENIRATAPITKNNHSVELSIG
NGLETQNNKLCAKLGNGLKENNGDICIKDSINTLWTGINPPPNCQIVENT
NINDGKLTLVLVKNGGLVNGYVSLVGVSDTVNQMFTQKTANIQLRLYFDS
SGNLLTEESDLKIPLKNKSSTATSETVASSKAFMPSTTAYPFNTTTRDSE
NYIHGICYYMTSYDRSLFPLNISIMLNSRMISSNVAYAIQFEWNLNASES
PESNIATLTTSPFFFSYITEDDN

GenBank Accession No. NC_011203 (SEQ ID NO: 199)

CTATCTATATAATATACCTTATAGATGGAATGGTGCCAACATGTAAATGAGGTAATTTAAAAAAGTGCGCGCTGTGT
GGTGATTGGCTGCGGGGTTAACGGCTAAAAGGGGCGGCGCGACCGTGGGAAAATGACGTGACTTATGTGGGAGGAGT
TATGTTGCAAGTTATTACGGTAAATGTGACGTAAAACGAGGTGTGGTTTGAACACGGAAGTAGACAGTTTTCCCACG
CTTACTGACAGGATATGAGGTAGTTTTGGGCGGATGCAAGTGAAAATTCTCCATTTTCGCGCGAAAACTAAATGAGG
AAGTGAATTTCTGAGTCATTTCGCGGTTATGCCAGGGTGGAGTATTTGCCGAGGGCCGAGTAGACTTTGACCGTTTA
CGTGGAGGTTTCGATTACCGTGTTTTTCACCTAAATTTCCGCGTACGGTGTCAAAGTTCTGTGTTTTTACGTAGGTG
TCAGCTGATCGCTAGGGTATTTAAACCTGACGAGTTCCGTCAAGAGGCCACTCTTGAGTGCCAGCGAGAAGAGTTTT
CTCCTCCGCGCCGCAAGTCAGTTCTGCGCTTTGAAAATGAGACACCTGCGCTTCCTGCCACAGGAGGTTATCTCCAG
TGAGACCGGGATCGAAATACTGGAGTTTGTGGTAAATACCCTAATGGGAGACGACCCGGAACCGCCAGTGCAGCCTT
TCGATCCACCTACGCTGCACGATCTGTATGATTTAGAGATAGACGGGCCGGAGGATCCCAATGAGGAAGCTGTGAAT
GGGTTTTTTACTGATTCTATGCTGCTAGCTGCTGATGAAGGATTGGACATAAACCCTCCTCCTGAGACACTTGTTAC
CCCAGGGGTGGTTGTGGAAAGCGGCATAGGTGGGAAAAAATTGCCTGATCTGGGAGCAGCTGAAATGGACTTGCGTT
GTTATGAAGAGGGTTTTCCTCCCAGTGATGATGAAGATGGGGAAACTGAGCAGTCCATCCATACCGCAGTAAATGAG
GGAGTAAAAGCTGCCAGCGATGTTTTTAAGTTGGACTGTCCGGAGCTGCCTGGACATGGCTGTAAGTCTTGTGAATT
TCACAGGAATAACACTGGAATGAAAGAACTATTGTGCTCGCTTTGCTATATGAGAATGCACTGCCACTTTATTTACA
GTAAGTGTATTTAAGTGAAATTTAAAGGAATAGTGTAGCTATTTAATAACTGTTGAATGGTAGATTTATGTTTTTTT
CTTGCGATTTTTTGTAGGTCCTGTGTCTGATGATGAGTCACCTTCTCCTGATTCAACTACCTCACCTCCTGAAATTC
AGGCGCCCGCACCTGCAAACGTATGCAAGCCCATTCCTGTGAAGCCTAAGCCTGGGAAACGCCCTGCTGTGGATAAG
CTTGAGGACTTGTTGGAGGGTGGGGATGGACCTTTGGACCTTAGTACCCGGAAACTGCCAAGGCAATGAGTGCCCTG
CAGCTGTGTTTATTTAATGTGACGTCATGTAATAAAATTATGTCAGCTGCTGAGTGTTTTATTACTTCTTGGGTGGG
GACTTGGATATATAAGTAGGAGCAGATCTGTGTGGTTAGCTCACAGCAACCTGCTGCCATCCATGGAGGTTTGGGCT
ATCTTGGAAGACCTCAGACAGACTAAGCTACTGCTAGAAAACGCCTCGGACGGAGTCTCTGGCCTTTGGAGATTCTG
GTTCGGTGGTGATCTAGCTAGGCTAGTGTTTAGGATAAAACAGGACTACAGGGAAGAATTTGAAAAGTTATTGGACG
ATAGTCCGGGACTTTTTGAAGCTCTTAACTTGGGTCATCAGGCTCATTTTAAGGAGAAGGTTTTATCAGTTTTAGAT
TTTTCTACTCCTGGTAGAACTGCTGCTGCTGTAGCTTTTCTTACTTTTATATTGGATAAATGGATCCGCCAAACTCA
CTTCAGCAAGGGATACGTTTTGGATTTCATAGCAGCAGCTTTGTGGAGAACATGGAAGGCTCGCAGGATGAGGACAA
TCTTAGATTACTGGCCAGTGCAGCCTCTGGGAGTAGCAGGGATACTGAGACACCCACCGACCATGCCAGCGGTTCTG
CAGGAGGAGCAGCAGGAGGACAATCCGAGAGCCGGCCTGGACCCTCCGGTGGAGGAGTAGCTGACCTGTTTCCTGAA
CTGCGACGGGTGCTTACTAGGTCTACGACCAGTGGACAGAACAGGGGAATTAAGAGGGAGAGGAATCCTAGTGGGAA
TAATTCAAGAACCGAGTTGGCTTTAAGTTTAATGAGCCGCAGGCGTCCTGAAACTGTTTGGTGGCATGAGGTTCAGA
GCGAAGGCAGGGATGAAGTTTCAATATTGCAGGAGAAATATTCACTAGAACAACTTAAGACCTGTTGGTTGGAACCT
GAGGATGATTGGGAGGTGGCCATTAGGAATTATGCTAAGATATCTCTGAGGCCTGATAAACAATATAGAATTACTAA
GAAGATTAATATTAGAAATGCATGCTACATATCAGGGAATGGGGCAGAGGTTATAATAGATACACAAGATAAAGCAG
TTTTTAGATGTTGTATGATGGGTATGTGGCCAGGGGTTGTCGGCATGGAAGCAGTAACACTTATGAATATTAGGTTT
AAAGGGGATGGGTATAATGGCATTGTATTTATGGCTAACACTAAGCTGATTCTACATGGTTGTAGCTTTTTTGGGTT
TAATAATACGTGTGTAGAAGCTTGGGGGCAAGTTAGTGTGAGGGGTTGTAGTTTTTATGCATGCTGGATTGCAACAT
CAGGTAGGGTCAAGAGTCAGTTGTCTGTGAAGAAATGCATGTTTGAGAGATGTAATCTTGGCATACTGAATGAAGGT
GAAGCAAGGGTCCGCCACTGCGCAGCTACAGAAACTGGCTGCTTCATTCTAATAAAGGGAAATGCCAGTGTGAAGCA
TAATATGATCTGTGGACATTCGGATGAGAGGCCTTATCAGATGCTGACCTGCGCTGGTGGACATTGCAATATTCTTG
CTACCGTGCATATCGTTTCACATGCACGCAAAAAATGGCCTGTATTTGAACATAATGTGATTACCAAGTGCACCATG
CACATAGGTGGTCGCAGGGGAATGTTTATGCCTTACCAGTGTAACATGAATCATGTGAAGGTAATGTTGGAACCAGA
TGCCTTTTCCAGAGTGAGCTTAACAGGAATCTTTGATATGAATATTCAACTATGGAAGATCCTGAGATATGATGACA
CTAAACCAAGGGTGCGCGCATGCGAATGCGGAGGCAAGCATGCTAGATTCCAGCCGGTGTGCGTGGATGTGACTGAA
GACTTGAGACCCGATCATTTGGTGCTTGCCTGCACTGGAGCGGAGTTCGGTTCTAGTGGTGAAGAAACTGACTAAAG
TGAGTAGTGGGGCAAGATGTGGATGGGGACTTTCAGGTTGGTAAGGTGGGCAGATTGGGTAAATTTTGTTAATTTCT
GTCTTGCAGCTGCCATGAGTGGAAGCGCTTCTTTTGAGGGGGGAGTATTTAGCCCTTATCTGACGGGCAGGCTCCCA
CCATGGGCAGGAGTTCGTCAGAATGTCATGGGATCCACTGTGGATGGGAGACCCGTCCAGCCCGCCAATTCCTCAAC
GCTGACCTATGCCACTTTGAGTTCGTCACCATTGGATGCAGCTGCAGCCGCCGCCGCTACTGCTGCCGCCAACACCA
TCCTTGGAATGGGCTATTATGGAAGCATCGTTGCCAATTCCAGTTCCTCTAATAACCCTTCAACCCTGGCTGAGGAC
AAGCTACTTGTTCTCTTGGCGCAGCTCGAGGCCTTAACCCAACGCTTAGGCGAACTGTCTAAGCAGGTGGCCCAGTT
GCGTGAGCAAACTGAGTCTGCTGTTGCCACAGCAAAGTCTAAATAAAGATCTCAAATCAATAAATAAAGAAATACTT
GATATAAAACAAATGAATGTTTATTTGATTTTTCGCGCGCGGTATGCCCTGGACCATCGGTCTCGATCATTGAGAAC
GCGGTGGATCTTTTCCAGTACCCTGTAAAGGTGGGATTGAATGTTTAGATACATGGGCATTAGTCCGTCTCGGGGGT
GGAGATAGCTCCATTGAAGAGCCTCTTGCTCCGGGGTAGTGTTATAAATCACCCAGTCATAGCAAGGTCGGAGTGCA
TGGTGTTGCACAATATCTTTTAGGAGCAGACTAATTGCAACGGGGAGGCCCTTAGTGTAGGTGTTTACAAATCTGTT
GAGCTGGGACGGGTGCATCCGGGGTGAAATTATATGCATTTTGGACTGGATCTTGAGGTTGGCAATGTTGCCGCCTA
GATCCCGTCTCGGGTTCATATTGTGCAGGACCACCAAGACAGTGTATCCGGTGCACTTGGGAAATTTATCATGCAGC
TTAGAGGGAAAAGCATGAAAAAATTTGGAGACGCCTTTGTGACCCCCCAGATTCTCCATGCACTCATCCATAATGAT
AGCGATGGGGCCGTGGGCAGCGGCACGGGCGAACACGTTCCGGGGGTCTGAAACATCATAGTTATGCTCCTGAGTCA
GGTCATCATAAGCCATTTTAATAAACTTTGGGCGGAGGGTGCCAGATTGGGGGATGAAAGTTCCCTCTGGCCCGGGA
GCATAGTTTCCCTCACATATTTGCATTTCCCAGGCTTTCAGTTCAGAGGGGGGGATCATGTCCACCTGCGGGGCTAT
AAAAAATACCGTTTCTGGAGCCGGGGTGATTAACTGGGACGAGAGCAAATTCCTAAGCAGCTGAGACTTGCCGCACC
CAGTGGGACCGTAAATGACCCCAATTACGGGTTGCAGATGGTAGTTTAGGGAACGACAGCTGCCGTCCTCCCGGAGC
AGGGGGGCCACTTCGTTCATCATTTCCCTTACATGGATATTTTCCCGCACCAAGTCCGTTAGGAGGCGCTCTCCCCC
AAGGGATAGAAGCTCCTGGAGCGAGGAGAAGTTTTTCAGCGGCTTCAGCCCGTCAGCCATGGGCATTTTGGAAAGAG
TCTGTTGCAAGAGCTCGAGCCGGTCCCAGAGCTCGGTGATGTGCTCTATGGCATCTCGATCCAGCAGACCTCCTCGT
TTCGCGGGTTGGGACGGCTCCTGGAGTAGGGAATCAGACGATGGGCGTCCAGCGCTGCCAGGGTCCGATCCTTCCAT
GGTCGCAGCGTCCGAGTCAGGGTTGTTTCCGTCACGGTGAAGGGGTGCGCGCCTGGTTGGGCGCTTGCGAGGGTGCG
CTTCAGACTCATCCTGCTGGTCGAGAACCGCTGCCGATTGGCGCCCTGCATGTCGGCCAGGTAGCAGTTTACCATGA
GTTCGTAGTTGAGCGCCTCGGCCGCGTGGCCTTTGGCACGGAGCTTACCTTTGGAAGTTTTATGGCAGGCGGGGCAG
TAGATACATTTGAGGGCATACAACTTGGGCGCGAGGAAAATGGATTCGGGGGAGTATGCATCCGCACCGCAGGAGGC
GCAGACGGTTTCGCACTCCACGAGCCAGGTCAGATCCGGCTCATCGGGGTCAAAAACAAGTTTTCCGCCATGTTTTT
TGATGCGTTTCTTACCTTTGGTTTCCATGAGTTCGTGTCCCCGCTGGGTGACAAAGAGGCTGTCCGTGTCCCCGTAG
ACCGACTTTATGGGCCTGTCCTCGAGCGGAGTGCCTCGGTCCTCTTCGTAGAGGAACCCAGCCCACTCTGATACAAA
AGCGCGTGTCCAGGCCAGCACAAAGGAGGCCACGTGGGAGGGGTAGCGGTCGTTGTCAACCAGGGGGTCCACCTTCT
CTACGGTATGTAAACACATGTCCCCCTCCTCCACATCCAAGAATGTGATTGGCTTGTAAGTGTAGGCCACGTGACCA
GGGGTCCCCGCCGGGGGGGTATAAAAGGGGGCGGGCCTCTGTTCGTCCTCACTGTCTTCCGGATCGCTGTCCAGGAG
CGCCAGCTGTTGGGGTAGGTATTCCCTCTCGAAGGCGGGCATGACCTCTGCACTCAGGTTGTCAGTTTCTAGGAACG
AGGAGGATTTGATATTGACAGTACCAGCAGAGATGCCTTTCATAAGACTCTCGTCCATCTGGTCAGAAAACACAATC
TTCTTGTTGTCCAGCTTGGTGGCAAATGATCCATAGAGGGCATTGGATAGAAGCTTGGCGATGGAGCGCATGGTTTG
GTTCTTTTCCTTGTCCGCGCGCTCCTTGGCGGTGATGTTAAGCTGGACGTACTCGCGCGCCACACATTTCCATTCAG
GAAAGATGGTTGTCAGTTCATCCGGAACTATTCTGATTCGCCATCCCCTATTGTGCAGGGTTATCAGATCCACACTG
GTGGCCACCTCGCCTCGGAGGGGCTCATTGGTCCAGCAGAGTCGACCTCCTTTTCTTGAACAGAAAGGGGGGAGGGG
GTCTAGCATGAACTCATCAGGGGGGTCCGCATCTATGGTAAATATTCCCGGTAGCAAATCTTTGTCAAAATAGCTGA
TGGTGGCGGGATCATCCAAGGTCATCTGCCATTCTCGAACTGCCAGCGCGCGCTCATAGGGGTTAAGAGGGGTGCCC
CAGGGCATGGGGTGGGTGAGCGCGGAGGCATACATGCCACAGATATCGTAGACATAGAGGGGCTCTTCGAGGATGCC
GATGTAAGTGGGATAACATCGCCCCCCTCTGATGCTTGCTCGCACATAGTCATAGAGTTCATGTGAGGGGGCAAGAA
GACCCGGGCCCAGATTGGTGCGGTTGGGTTTTTCCGCCCTGTAAACGATCTGGCGAAAGATGGCATGGGAATTGGAA
GAGATAGTAGGTCTCTGGAATATGTTAAAATGGGCATGAGGTAAGCCTACAGAGTCCCTTATGAAGTGGGCATATGA
CTCTTGCAGCTTGGCTACCAGCTCGGCGGTGATGAGTACATCCAGGGCACAGTAGTCGAGAGTTTCCTGGATGATGT
CATAACGCGGTTGGCTTTTCTTTTCCCACAGCTCGCGGTTGAGAAGGTATTCTTCGTGATCCTTCCAGTACTCTTCG
AGGGGAAACCCGTCTTTTTCTGCACGGTAAGAGCCCAACATGTAGAACTGATTGACTGCCTTGTAGGGACAGCATCC
CTTCTCCACTGGGAGAGAGTATGCTTGGGCTGCATTGCGCAGCGAGGTATGAGTGAGGGCAAAAGTGTCCCTGACCA
TGACTTTGAGGAATTGATACTTGAAGTCGATGTCATCACAGGCCCCCTGTTCCCAGAGTTGGAAGTCCACCCGCTTC
TTGTAGGCGGGGTTGGGCAAAGCGAAAGTAACATCATTGAAGAGGATCTTGCCGGCCCTGGGCATGAAATTTCGGGT
GATTTTGAAAGGCTGAGGAACCTCTGCTCGGTTATTGATAACCTGAGCGGCCAAGACGATCTCATCAAAGCCATTGA
TGTTGTGCCCCACTATGTACAGTTCTAAGAATCGAGGGGTGCCCCTGACATGAGGCAGCTTCTTGAGTTCTTCAAAA
GTGAGATCTGTAGGGTCAGTGAGAGCATAGTGTTCGAGGGCCCATTCGTGCACGTGAGGGTTCGCTTTAAGGAAGGA
GGACCAGAGGTCCACTGCCAGTGCTGTTTGTAACTGGTCCCGGTACTGACGAAAATGCTGTCCGACTGCCATCTTTT
CTGGGGTGACGCAATAGAAGGTTTGGGGGTCCTGCCGCCAGCGATCCCACTTGAGTTTTATGGCGAGGTCATAGGCG
ATGTTGACGAGCCGCTGGTCTCCAGAGAGTTTCATGACCAGCATGAAGGGGATTAGCTGCTTGCCAAAGGACCCCAT
CCAGGTGTAGGTTTCCACATCGTAGGTGAGAAAGAGCCTTTCTGTGCGAGGATGAGAGCCAATCGGGAAGAACTGGA
TCTCCTGCCACCAGTTGGAGGAATGGCTGTTGATGTGATGGAAGTAGAACTCCCTGCGACGCGCCGAGCATTCATGC
TTGTGCTTGTACAGACGGCCGCAGTACTCGCAGCGATTCACGGGATGCACCTTATGAATGAGTTGTACCTGACTTCC
TTTGACGAGAAATTTCAGTGGAAAATTGAGGCCTGGCGCTTGTACCTCGCGCTTTACTATGTTGTCTGCATCGGCAT
GACCATCTTCTGTCTCGATGGTGGTCATGCTGACGAGCCCTCGCGGGAGGCAAGTCCAGACCTCGGCGCGGCAGGGG
CGGAGCTCGAGGACGAGAGCGCGCAGGCCGGAGCTGTCCAGGGTCCTGAGACGCTGCGGAGTCAGGTTAGTAGGCAG
TGTCAGGAGATTAACTTGCATGATCTTTTGGAGGGCGTGAGGGAGGTTCAGATAGTACTTGATCTCAACGGGTCCGT
TGGTGGAGATGTCGATGGCTTGCAGGGTTCCGTGTCCCTTGGGCGCTACCACCGTGCCCTTGTTTTTCATTTTGGAC
GGCGGTGGCTCTGTTGCTTCTTGCATGTTTAGAAGCGGTGTCGAGGGCGCGCACCGGGCGGCAGGGGCGGCTCGGGA
CCCGGCGGCATGGCTGGCAGTGGTACGTCGGCGCCGCGCGCGGGTAGGTTCTGGTACTGCGCCCTGAGAAGACTCGC
ATGCGCGACGACGCGGCGGTTGACATCCTGGATCTGACGCCTCTGGGTGAAAGCTACCGGCCCCGTGAGCTTGAACC
TGAAAGAGAGTTCAACAGAATCAATCTCGGTATCGTTGACGGCGGCTTGCCTAAGGATTTCTTGCACGTCGCCAGAG
TTGTCCTGGTAGGCGATCTCGGCCATGAACTGCTCGATCTCTTCCTCTTGAAGATCTCCGCGGCCCGCTCTCTCGAC
GGTGGCCGCGAGGTCGTTGGAGATGCGCCCAATGAGTTGAGAGAATGCATTCATGCCCGCCTCGTTCCAGACGCGGC
TGTAGACCACAGCCCCCACGGGATCTCTCGCGCGCATGACCACCTGGGCGAGGTTGAGCTCCACGTGGCGGGTGAAG
ACCGCATAGTTGCATAGGCGCTGGAAAAGGTAGTTGAGTGTGGTGGCGATGTGCTCGGTGACGAAGAAATACATGAT
CCATCGTCTCAGCGGCATCTCGCTGACATCGCCCAGCGCTTCCAAGCGCTCCATGGCCTCGTAGAAGTCCACGGCAA
AGTTAAAAAACTGGGAGTTACGCGCGGACACGGTCAACTCCTCTTCCAGAAGACGGATAAGTTCGGCGATGGTGGTG
CGCACCTCGCGCTCGAAAGCCCCTGGGATTTCTTCCTCAATCTCTTCTTCTTCCACTAACATCTCTTCCTCTTCAGG
TGGGGCTGCAGGAGGAGGGGGAACGCGGCGACGCCGGCGGCGCACGGGCAGACGGTCGATGAATCTTTCAATGACCT
CTCCGCGGCGGCGGCGCATGGTCTCGGTGACGGCACGACCGTTCTCCCTGGGTCTCAGAGTGAAGACGCCTCCGCGC
ATCTCCCTGAAGTGGTGACTGGGAGGCTCTCCGTTGGGCAGGGACACCGCGCTGATTATGCATTTTATCAATTGCCC
CGTAGGTACTCCGCGCAAGGACCTGATCGTCTCAAGATCCACGGGATCTGAAAACCTTTCGACGAAAGCGTCTAACC
AGTCGCAATCGCAAGGTAGGCTGAGCACTGTTTCTTGCGGGCGGGGGCGGCTAGACGCTCGGTCGGGGTTCTCTCTT
TCTTCTCCTTCCTCCTCTTGGGAGGGTGAGACGATGCTGCTGGTGATGAAATTAAAATAGGCAGTTTTGAGACGGCG
GATGGTGGCGAGGAGCACCAGGTCTTTGGGTCCGGCTTGTTGGATACGCAGGCGATGAGCCATTCCCCAAGCATTAT
CCTGACATCTGGCCAGATCTTTATAGTAGTCTTGCATGAGTCGTTCCACGGGCACTTCTTCTTCGCCCGCTCTGCCA
TGCATGCGAGTGATCCCGAACCCGCGCATGGGCTGGACAAGTGCCAGGTCCGCTACAACCCTTTCGGCGAGGATGGC
TTGCTGCACCTGGGTGAGGGTGGCTTGGAAGTCGTCAAAGTCCACGAAGCGGTGGTAGGCCCCGGTGTTGATTGTGT
AGGAGCAGTTGGCCATGACTGACCAGTTGACTGTCTGGTGCCCAGGGCGCACGAGCTCGGTGTACTTGAGGCGCGAG
TATGCGCGGGTGTCAAAGATGTAATCGTTGCAGGTGCGCACCAGGTACTGGTAGCCAATGAGAAAGTGTGGCGGTGG
CTGGCGGTACAGGGGCCATCGCTCTGTAGCCGGGGCTCCGGGGGCGAGGTCTTCCAGCATGAGGCGGTGGTAGCCGT
AGATGTACCTGGACATCCAGGTGATACCGGAGGCGGTGGTGGATGCACGTGGGAACTCGCGCACGCGGTTCCAGATG
TTGCGCAGCGGCATGAAGTAGTTCATGGTAGGCACGGTCTGGCCAGTGAGGCGCGCGCAGTCATTGACGCTCTGTAG
ACACGGAGAAAACGAAAGCGATGAGCGGCTCGACTCCGTGGTCTGGGGGAACGTGAACGGGTTGGGTCGCGGTGTAC
CCCGGTTCGAGTCCAAAGCTAAGCGATCACGCTCGGATCGGCCGGAGCCGCGGCTAACGTGGTATTGGCTATCCCGT
CTCGACCCAGCCGACGAATATCCAGGGTACGGAGTAGAGTCGTTTTTGCTGCTTTTTCCTGGACGTGTGCCATTGCC
ACGTCAAGCTTTAGAACGCTCAGTTCTCGGGCCGTGAGTGGCTCGCGCCCGTAGTCTGGAGAATCAGTCGCCAGGGT
TGCGTTGCGGTATGCCCCGGTTGGAGCCTAAGCGCGGCTCGTATCGGCCGGTTTCCGCGACAAGCGAGGGTTTGGCA
GCCCCGTTATTTCCAAGACCCCGCCAGCCGACTTCTCCAGTTTACGGGAGCGAGCCCTTTTTTTTTTTTTTTTGTTT
TTGTCGCCCAGATGCATCCAGTGCTGCGACAGATGCGCCCCCAGCAACAGGCCCCTTCTCAGCAACAGCCACAAAAG
GCTCTTCTTGCTCCTGCAACTACTGCAGCTGCAGCCGTGAGCGGCGCGGGACAGCCCGCCTATGATCTGGACTTGGA
AGAGGGCGAGGGATTGGCGCGCCTGGGGGCTCCATCGCCCGAGCGGCACCCGCGGGTGCAACTAAAAAAGGACTCTC
GCGAGGCGTACGTGCCCCAGCAGAACCTGTTCAGGGACAGGAGCGGCGAGGAGCCAGAGGAGATGCGAGCATCTCGA
TTTAACGCGGGTCGCGAGCTGCGCCACGGTCTGGATCGAAGACGGGTGCTGCAAGACGAGGATTTTGAGGTCGATGA
AGTCACAGGGATCAGCCCAGCTAGGGCACATGTGGCCGCGGCCAACCTAGTCTCGGCCTACGAGCAGACCGTGAAGG
AGGAGCGCAACTTCCAAAAATCTTTTAACAACCATGTGCGCACCCTGATCGCCCGCGAGGAAGTGACCCTGGGTCTG
ATGCATCTGTGGGACCTGATGGAGGCTATCGCCCAAAACCCCACTAGCAAACCACTGACAGCTCAGCTGTTTCTGGT
GGTTCAACATAGCAGGGACAACGAGGCATTCAGGGAGGCGTTGTTGAACATCACCGAGCCTGATGGGAGATGGCTGT
ATGATCTGATCAACATCCTGCAAAGTATTATAGTGCAGGAACGTAGCCTGGGTTTGGCTGAGAAAGTGGCAGCTATC
AACTACTCGGTCTTGAGCCTGGGCAAATACTACGCTCGCAAGATCTACAAGACCCCCTACGTACCCATAGACAAGGA
GGTGAAGATAGATGGGTTTTACATGCGCATGACTCTGAAGGTGCTGACTCTGAGCGACGATCTGGGGGTGTATCGCA
ATGACAGGATGCACCGCGCGGTGAGCGCCAGCAGGAGGCGCGAGCTGAGCGACAGAGAACTTATGCACAGCTTGCAA
AGAGCTCTAACGGGGGCCGGGACTGATGGGGAGAACTACTTTGACATGGGAGCGGATTTGCAATGGCAACCCAGTCG
CAGGGCCATGGAGGCTGCAGGGTGTGAGCTTCCTTACATAGAAGAGGTGGATGAAGTCGAGGACGAGGAGGGCGAGT
ACTTGGAAGACTGATGGCGCGACCCGTATTTTTGCTAGATGGAACAGCAGCAGGCACCGGACCCCGCAATGCGGGCG
GCGCTGCAGAGCCAGCCGTCCGGCATTAACTCCTCGGACGATTGGACCCAGGCCATGCAACGCATAATGGCGCTGAC
GACCCGCAACCCCGAAGCCTTTAGACAGCAACCCCAGGCCAACCGCCTTTCTGCCATACTGGAGGCCGTAGTGCCCT
CCCGCTCCAACCCCACCCACGAGAAGGTCCTGGCTATCGTGAACGCGCTGGTGGAGAACAAGGCCATCCGTCCCGAT
GAGGCCGGGCTGGTATACAATGCTCTCTTGGAGCGCGTGGCCCGTTACAACAGCAGCAACGTGCAAACCAACCTGGA
CCGGATGGTGACCGATGTGCGCGAGGCCGTGTCTCAGCGCGAGCGATTCCAGCGCGACGCCAACTTGGGGTCGTTGG
TAGCGCTAAACGCTTTCCTCAGCACCCAGCCCGCCAACGTGCCCCGTGGTCAGCAAGACTATACAAACTTTTTGAGT
GCATTGAGACTCATGGTAGCTGAGGTGCCCCAGAGCGAGGTGTACCAGTCCGGGCCAGATTACTTCTTCCAGACCAG
CAGACAGGGCTTGCAGACAGTGAACCTGACTCAGGCTTTCAAGAACCTGAAGGGTCTGTGGGGAGTGCACGCCCCAG
TAGGGGATCGCGCGACCGTGTCTAGCTTGCTGACTCCCAACTCCCGCCTGCTGCTGCTGCTGGTATCCCCCTTTACT
GACAGCGGTAGCATTGACCGCAACTCGTACTTGGGCTACCTGCTTAACCTGTATCGCGAGGCCATAGGACAGAGCCA
GGTGGACGAGCAGACCTATCAAGAAATCACCCAAGTGAGCCGCGCCCTGGGTCAGGAAGACACGGGCAGTTTGGAAG
CCACCCTGAACTTCTTGCTAACCAACCGGTCACAGAAGATCCCTCCTCAGTATGCGCTTACCGCTGAGGAGGAGCGG
ATCCTCAGATACGTGCAACAGAGCGTTGGACTGTTCCTGATGCAGGAGGGGGCGACACCTACCGCCGCGCTGGACAT
GACAGCTCGAAACATGGAGCCCAGCATGTATGCTAGTAACAGGCCTTTCATTAACAAACTGCTGGACTACCTGCACA
GGGCGGCCGCCATGAACTCTGATTATTTCACCAATGCTATCCTGAACCCACACTGGCTGCCCCCACCTGGTTTCTAC
ACTGGCGAGTACGACATGCCCGACCCCAATGACGGGTTCCTGTGGGACGATGTGGACAGCAGCATATTTTCCCCGCC
TCCCGGTTATACAGTTTGGAAGAAGGAAGGGGGCGATAGAAGACACTCTTCCGTGTCGCTATCCGGAACGGCTGGTG
CTGCCGCGACCGTGCCCGAAGCTGCAAGTCCTTTCCCTAGCTTGCCCTTTTCACTAAACAGCGTTCGCAGCAGTGAA
CTGGGGAGAATAACCCGCCCGCGCTTGATGGGCGAGGATGAGTACTTGAATGACTCTTTGCTGAGGCCAGAGAGGGA
AAAGAACTTCCCCAACAATGGAATAGAGAGTCTGGTGGATAAGATGAGTAGATGGAAGACCTATGCGCAGGATCACA
GAGACGAGCCCAGGATCTTGGGGGCTACAAGCAGACCGATCCGTAGACGCCAGCGCCACGACAGGCAGATGGGTCTT
GTGTGGGACGATGAGGACTCTGCCGATGATAGCAGCGTGTTGGACTTGGGTGGAAGAGGAGGGGGCAACCCGTTCGC
TCATCTGCGTCCCAGATTCGGGCGCATGTTGTAAAAGTGAAAGTAAAATAAAAAGGCAACTCACCAAGGCCATGGCG
ACCGAGCGTGCGTTCGTTCTTTTTTGTTATCTGTGTCTAGTACGATGAGGAGACGAGCCGTGCTAGGCGGAGCGGTG
GTGTATCCGGAGGGTCCTCCTCCTTCTTACGAGAGCGTGATGCAGCAACAGGCGGCGATGATACAGCCCCCACTGGA
GGCTCCCTTCGTACCCCCACGGTACCTGGCGCCTACGGAAGGGAGAAACAGCATTCGTTACTCGGAGCTGTCGCCCC
TGTACGATACCACCAAGTTGTATCTGGTGGACAACAAGTCGGCGGACATCGCCTCCCTGAACTATCAGAACGACCAC
AGCAACTTCCTGACCACGGTGGTGCAGAACAATGACTTTACCCCCACGGAGGCTAGCACCCAGACCATCAACTTTGA
CGAGCGGTCGCGATGGGGCGGTCAGCTGAAGACCATCATGCACACCAACATGCCCAACGTGAACGAGTACATGTTCA
GCAACAAGTTCAAGGCGAGGGTGATGGTGTCCAGAAAAGCTCCTGAAGGTGTTACAGTAAATGACACCTATGATCAT
AAAGAGGATATCTTGAAGTATGAGTGGTTTGAGTTCATTTTACCAGAAGGCAACTTTTCAGCCACCATGACGATCGA
CCTGATGAACAATGCCATCATTGACAACTACCTGGAAATTGGCAGACAGAATGGAGTGCTGGAAAGTGACATTGGTG
TTAAGTTTGACACTAGAAATTTCAGGCTCGGGTGGGACCCCGAAACTAAGTTGATTATGCCAGGTGTCTACACTTAT
GAGGCATTCCATCCTGACATTGTATTGCTGCCTGGTTGCGGGGTAGACTTTACTGAAAGCCGACTTAGCAACTTGCT
TGGCATCAGGAAGAGACATCCATTCCAGGAGGGTTTCAAAATCATGTATGAAGATCTTGAAGGGGGTAATATTCCTG
CCCTTTTGGATGTCACTGCCTATGAGGAAAGCAAAAAGGATACCACTACTGAAACAACCACACTGGCTGTTGCAGAG
GAAACTAGTGAAGATGATGATATAACTAGAGGAGATACCTATATAACTGAAAAACAAAAACGTGAAGCTGCAGCTGC
TGAAGTTAAAAAAGAGTTAAAGATCCAACCTCTAGAAAAAGACAGCAAGAGTAGAAGCTACAATGTCTTGGAAGACA
AAATCAACACAGCCTACCGCAGTTGGTACCTGTCCTACAATTACGGTAACCCTGAGAAAGGAATAAGGTCTTGGACA
CTGCTCACCACTTCAGATGTCACCTGTGGGGCAGAGCAGGTCTACTGGTCGCTCCCTGACATGATGCAAGACCCAGT
CACCTTCCGCTCCACAAGACAAGTCAACAACTACCCAGTGGTGGGTGCAGAGCTTATGCCCGTCTTCTCAAAGAGTT
TCTACAATGAGCAAGCCGTGTACTCTCAGCAGCTCCGACAGGCCACTTCGCTCACGCACGTCTTCAACCGCTTCCCT
GAGAACCAGATCCTCATCCGCCCGCCGGCGCCCACAATTACCACCGTCAGTGAAAACGTTCCTGCTCTCACAGATCA
CGGGACCCTGCCGTTACGCAGCAGTATCCGGGGAGTCCAGCGCGTGACCGTTACTGACGCCAGACGCCGCACCTGTC
CCTACGTTTACAAGGCCCTGGGCATAGTCGCGCCGCGCGTTCTTTCAAGCCGCACTTTCTAAAAAAAAAAAAAATGT
CCATTCTCATCTCGCCCAGTAATAATACCGGTTGGGGACTGTATGCGCCCACCAAGATGTATGGAGGCGCCCGCAAG
CGCTCTACCCAGCATCCTGTGCGCGTTCGCGGTCATTTCCGCGCTCCCTGGGGCGCACTCAAGGGTCGTACCCGCAC
TCGGACCACGGTCGATGATGTGATCGACCAGGTGGTCGCCGATGCTCGTAATTATACTCCTACTGCGCCTACATCTA
CTGTGGATGCAGTTATTGACAGTGTGGTGGCAGACGCCCGCGCCTATGCTCGCCGGAAGAGCCGAAGGAGGCGCATC
GCCAGGCGCCACAGGGCTACTCCCGCCATGCGAGCCGCAAAAGCTATTCTGCGGAGGGCCAAACGTGTGGGGCGAAG
AGCCATGCTTAGAGCGGCCAGACGCGCGGCTTCAGGTGCCAGCAGCGGCAGGTCCCGCAGGCGCGCGGCCACGGCGG
CAGCAGCGGCCATTGCCAACATGGCCCAACCGCGAAGAGGCAATGTGTACTGGGTGCGTGATGCCACTACCGGCCAG
CGCGTGCCCGTGCGCACCCGCCCCCCTCGCACTTAGAAGATACTGAGCAGTCTCCGATGTTGTGTCCCAGCGGCAAG
TATGTCCAAGCGCAAATACAAGGAAGAGATGCTCCAGGTCATCGCGCCTGAAATCTACGGTCCGCCGGTGAAGGATG
AAAAAAAGCCCCGCAAAATCAAGCGGGTCAAAAATAACAAAAAGGAAGAAGATGACGATGATGGGCTGGTGGAGTTT
GTGCGCGAGTTCGCCCCAAGACGGCGCGTGCAGTGGCGCGGGCGCAAAGTGCGTCAAGTGCTCAGACCCGGGACCAC
TGTGGTTTTTACACCTGGCGAGCGTTCCAGCACTACTTTTAAACGGTCCTATGATGAGGTGTACGGGGATGACGATA
TTCTTGAGCAGGCGGCAGACCGCCTTGACGAGTTTGCTTATGGCAAGCGCACTAGATCCAGTCCCAAAGAGGAGGCT
GTGTCCATTCCTTTGGATCATGGAAATCCCACCCCCAGCCTCAAACCAGTCACCCTGCAGCAAGTGCTGCCCGTGCC
TGCGCGGAGAGGCGTAAAGCGCGAGGGTGAGGACCTATATCCCACCATGCAGCTAATGGTGCCCAAGCGCCAGAGGC
TAGAAGACGTACTGGAGAAAATGAAAGTGGATGCCGATATCCAGCCTGAGGTCAAAGTAAGACCTATCAAGGAAGTG
GCGCCAGGTTTGGGAGTACAAACCTTCGACATCAAGATTCCCACCGAGTCCATGGAAGTGCAGACCGAACCTGCAAA
ACCCACCACCTCAATTGAGGTGCAAACGGAACCCTGGATGCCCGCGCCCGTTGCCGCCCCCAGCACCACTCGAAGAT
CACGACGAAAGTACGGCCCAGCAAGTCTGCTAATGCCCAACTATGCTCTGCACCCATCCATCATTCCCACTCCGGGT
TACAGAGGCACTCGCTACTATCGAAACCGGAGCAACACCTCTCGCCGCCGCAAACCACCTGCAAGTCGCACTCGCCG
TCGCCGCCGCCGCAACACTGCCAGCAAATTGACTCCCGCCGCCCTGGTGCGGAGAGTGTACCGCGATGGTCGCGCTG
AACCTCTGACGCTGCCGCGCGCGCGCTACCATCCAAGCATCACCACTTAATGACTGTTGACGCTGCCTCCTTGCAGA
TATGGCCCTCACTTGCCGCCTTCGCGTCCCCATTACTGGCTACCGAGGAAGAAACTCGCGCCGTAGAAGGATGTTGG
GGCGAGGGATGCGCCGCCACAGACGAAGGCGCGCTATCAGCAGACGATTAGGGGGTGGCTTTTTGCCAGCTCTTATA
CCCATCATCGCCGCAGCGATCGGGGCGATACCAGGCATAGCTTCAGTGGCGGTTCAGGCCTCGCAGCGCCACTAACA
TTGGAAAAAACTTATAAATAAAAAATAGAATGGACTCTGACGCTCCTGGTCCTGTGACTATGTTTTTGTAGAGATGG
AAGACATCAATTTTTCATCCCTGGCTCCGCGACACGGCACGAGGCCGTACATGGGCACCTGGAGCGACATCGGCACG
AGCCAACTGAACGGGGGCGCCTTCAATTGGAGCAGTATCTGGAGCGGGCTTAAAAATTTTGGCTCGACCGTAAAAAC
CTATGGGAACAAAGCTTGGAACAGCAGCACAGGGCAGGCTCTGAGAAATAAGCTTAAGGAACAAAACTTCCAACAGA
AGGTGGTCGATGGGATCGCCTCTGGTATTAACGGCGTAGTGGATCTGGCCAACCAGGCTGTACAAAAACAGATAAAC
AGCCGCCTGGACCCGCCGCCCGCAACCCCTGGTGAAATGGAAGTGGAGGAAGAACTTCCTCCGCTGGAAAAGCGGGG
CGACAAGCGTCCGCGACCCGAGCTAGAGCAGACGCTGGTGACGCGCGCAGACGAGCCCCCTTCATATGAGGAGGCAG
TAAAGCTCGGAATGCCCACTACCAGGCCTGTAGCTCACATGGCTACCGGGGTGATGAAACCTTCTCAGTCACATCGA
CCCGCCACCTTGGACTTGCCTCCTCCCCCTGCTTCTGCGGCGCCTGTTCCCAAACCTGTCGCTACCAGAAAGCCCAC
CGCCGTACAGCCCGTCGCCGTAGCCAGACCGCGTCCTGGGGGCACACCGCGCCCGAAAGCAAACTGGCAAAGTACTC
TGAACAGCATCGTGGGTCTGGGCGTGCAGAGTGTAAAGCGCCGTCGCTGCTATTAATTAAATATGGAGTAGCGCTTA
ACTTGCTTGTCTGTGTGTATGTATCATCACCACGCCGCCGCAGCAGAGGAGAAAGGAAGAGGTCGCGCGCCGAGGCT
GAGTTGCTTTCAAGATGGCCACCCCATCGATGATGCCCCAATGGGCATACATGCACATCGCCGGACAGGATGCTTCG
GAGTACCTCAGTCCGGGTCTGGTGCAGTTCGCCCGTGCAACAGACACCTACTTCAGTATGGGGAACAAATTTAGAAA
CCCCACAGTGGCGCCCACCCACGATGTGACCACCGACCGTAGCCAGCGCCTGATGCTGCGCTTCGTGCCCGTTGACC
GGGAAGACAATACCTACTCTTACAAAGTTCGCTACACGCTGGCTGTAGGCGACAACAGAGTGCTTGACATGGCCAGC
ACATTCTTTGACATTCGGGGGGTGCTTGATAGAGGTCCTAGCTTCAAGCCATATTCCGGCACAGCTTACAATTCACT
CGCTCCTAAGGGCGCGCCCAATACATCTCAGTGGATAGTTACAACAAATGGGGACAATGCAGTAACTACCACCACAA
ACACATTTGGCATTGCTTCCATGAAGGGAGACAATATTACTAAAGAAGGTTTGCAAATTGGGAAAGACATTACCACT
ACTGAAGGAGAAGAAAAGCCCATTTATGCCGATAAAACATATCAGCCAGAGCCTCAAGTTGGAGAAGAATCATGGAC
TGATACTGATGGAACAAATGAAAAGTTTGGTGGAAGAGCCCTTAAACCAGCTACCAACATGAAGCCATGCTACGGGT
CTTTTGCAAGACCTACAAACATAAAAGGGGGCCAAGCTAAAAACAGAAAAGTAAAACCAACAACCGAAGGAGGGGTT
GAAACTGAGGAACCAGATATTGATATGGAATTTTTCGATGGTAGAGATGCTGTTGCAGGAGCTTTAGCGCCTGAAAT
TGTGCTTTATACGGAAAATGTAAATTTGGAAACTCCAGACAGTCATGTGGTATATAAACCAGAAACGTCTAATAACT
CTCATGCAAATTTGGGTCAACAAGCCATGCCTAACAGACCCAATTACATTGGCTTCAGGGATAACTTCGTAGGCCTA
ATGTACTACAACAGTACTGGAAATATGGGAGTTTTGGCTGGCCAAGCATCACAACTGAATGCAGTGGTTGACTTGCA
GGACAGAAATACTGAACTGTCATATCAGCTTTTGCTTGATTCTCTGGGAGACAGAACCAGATACTTCAGCATGTGGA
ATCAGGCTGTGGACAGTTACGATCCCGATGTTCGCATTATTGAAAATCATGGCATCGAGGATGAACTGCCTAATTAC
TGTTTTCCTCTGAATGGCATAGGACCAGGGCACACATATCAAGGCATTAAAGTTAAAACCGATGACACTAATGGATG
GGAAAAAGATGCTAATGTTGCTCCAGCTAATGAAATAACCATAGGCAACAACCTGGCTATGGAAATTAATATCCAAG
CTAACCTTTGGAGAAGTTTTCTGTACTCTAATGTGGCTTTGTACCTTCCAGATGTTTACAAGTACACGCCACCTAAC
ATTACTTTGCCCACTAACACCAACACCTATGAGTACATGAACGGGCGAGTGGTATCCCCATCCCTGGTTGATTCATA
CATCAACATTGGCGCCAGGTGGTCTCTTGACCCAATGGACAATGTGAATCCATTCAACCACCACCGCAATGCTGGTC
TGCGCTACAGGTCCATGCTTCTGGGAAATGGTCGTTATGTGCCTTTCCACATACAAGTGCCTCAGAAATTCTTTGCT
GTCAAGAACCTACTTCTTCTACCTGGCTCCTACACCTACGAGTGGAACTTCCGAAAGGATGTGAACATGGTCCTGCA
AAGTTCCCTTGGAAATGACCTCAGAACGGATGGTGCTACCATAAGTTTCACCAGCATCAATCTCTATGCCACCTTCT
TCCCCATGGCTCACAACACAGCTTCCACCCTTGAAGCCATGCTGCGCAACGATACCAATGATCAGTCATTTAACGAC
TACCTCTCTGCAGCTAACATGCTTTACCCCATTCCTGCCAATGCAACCAACATTCCAATTTCCATCCCATCTCGCAA
CTGGGCAGCCTTCAGGGGCTGGTCCTTCACCAGACTCAAAACCAAGGAGACTCCATCTCTTGGATCAGGGTTCGATC
CCTACTTCGTATATTCTGGATCTATTCCCTACCTGGATGGCACCTTTTACCTTAACCACACTTTCAAGAAGGTCTCC
ATCATGTTTGACTCCTCAGTCAGCTGGCCTGGCAATGACAGGCTGTTGAGTCCAAATGAGTTTGAAATCAAGCGCAC
TGTGGACGGGGAAGGATACAACGTGGCACAATGCAACATGACCAAAGACTGGTTCCTGGTTCAGATGCTTGCCAATT
ACAACATTGGCTACCAGGGCTTTTACATCCCTGAGGGATACAAGGATCGCATGTACTCCTTTTTCAGAAACTTCCAG
CCTATGAGCAGGCAGGTGGTTGATGAGGTTAATTACACTGACTACAAAGCCGTCACCTTACCATACCAACACAACAA
CTCTGGCTTTGTAGGGTACCTTGCACCTACTATGAGACAAGGGGAACCTTACCCAGCCAATTATCCATACCCGCTCA
TCGGAACTACTGCCGTTAAGAGTGTCACCCAGAAAAAGTTCCTGTGCGACAGGACCATGTGGCGCATTCCCTTCTCC
AGCAACTTCATGTCCATGGGGGCCCTTACCGACCTGGGACAGAACATGCTCTATGCCAACTCAGCCCATGCGCTGGA
CATGACTTTTGAGGTGGATCCCATGGATGAGCCCACCCTGCTTTATCTTCTTTTCGAAGTCTTCGACGTGGTCAGAG
TGCACCAGCCACACCGCGGCGTCATCGAGGCCGTCTACCTGCGCACACCGTTCTCGGCCGGCAACGCCACCACATAA
GAAGCCTCTTGCTTCTTGCAAGCAGCAGCTGCAGCCATGACATGCGGGTCCGGAAACGGCTCCAGCGAGCAAGAGCT
CAAAGCCATCGTCCGAGACCTGGGCTGCGGACCCTATTTCCTGGGAACCTTTGACAAGCGTTTCCCGGGGTTCATGG
CCCCCGACAAGCTCGCCTGCGCCATAGTCAACACTGCCGGACGCGAGACGGGGGGAGAGCACTGGCTGGCTTTTGGT
TGGAACCCGCGCTACAACACCTGCTACCTTTTTGATCCTTTTGGGTTCTCGGATGAGCGGCTCAAACAGATTTACCA
GTTTGAGTACGAGGGGCTCCTGCGTCGCAGTGCCCTTGCTACCAAAGACCGCTGCATCACCCTGGAGAAGTCTACCC
AAAGCGTGCAGGGTCCGCGCTCAGCCGCCTGTGGACTTTTTTGCTGTATGTTCCTTCATGCCTTTGTGCACTGGCCC
GACCGCCCCATGGACGGAAACCCCACCATGAAGTTGCTGACTGGGGTGTCCAACAGCATGCTCCAATCACCCCAAGT
CCAGCCCACCCTGCGCCGCAACCAGGAGGTGCTATACCGCTTCCTAAACACCCACTCATCTTACTTTCGTTCTCACC
GCGCGCGCATTGAAAGGGCCACCGCGTTTGACCGTATGGATATGCAATAAGTCATGTAAAACCGTGTTCAATAAACA
GCACTTTATTTTTACATGCACTGAGGCTCTGGTTTTGCTCATTTGTTTCATCATTTACTCAGAAGTCGAATGGGTTC
TGGCGGGAGTCAGAGTGACCCGCGGGCAGGGATACGTTGCGGAACTGTAACCTGTTCTGCCACTTGAACTCGGGGAT
TACCAGCTTGGGAACTGGAATCTCGGGAAAGGTGTCTTGCCACAACTTTCTGGTCAGTTGCATAGCGCCAAGCAGGT
CAGGAGCAGAGATCTTGAAATCACAGTTGGGGCCGGCATTCTGGACACGGGAGTTGCGATACACTGGGTTGCAACAC
TGGAACACTATCAACGCTGGGTGTCTTACGCTTGCCAACACGGTTGGGTCACTGATGGTAGTCACATCCAAGTCTTC
AGCATTGGCCATCCCAAAGGGGGTCATCTTACATGTCTGCCTGCCCATCACGGGAGCGCAGCCTGGCTTGTGGTTGC
AATCACAATGAATGGGGATCAGCATCATCCTGGCTTGGTCGGGAGTTATCCCTGGGTACACAGCCTTCATGAAGGCT
TCGTACTGCTTAAAAGCTTCCTGGGCCTTACTTCCCTCGGTGTAGAACATCCCACAGGACTTGCTGGAAAATTGATT
AGTAGTACAGTTGGCATCATTCACACAACAGCGGGCATCGTTGTTGGCCAACTGAACCACATTTCTGCCCCAGCGGT
TTTGGGTGATCTTGGCTCTGTCTGGATTCTCCTTCATAGCGCGCTGCCCGTTCTCGCTCGCCACATCCATCTCGATA
ATGTGGTCCTTCTGGATCATGATAGTGCCATGCAGGCATTTCACCTTGCCTTCATAATCGGTGCATCCATGAGCCCA
CAGAGCGCACCCGGTGCACTCCCAATTATTGTGGGCGATCTCAGAATAATAATGTACCAATCCCTGCATGAATCTTC
CCATCATTGTTGTCAAGGTCTTCATGCTGGTAAATGTCAGCGGGATGCCACGGTGCTCCTCGTTCACATACTGGTGG
CAGATACGCTTGTATTGCTCGTGCTGCTCTGGCATCAGCTTGAAAGAGGTTCTCAGATCATTATCCAGCCTGTACCT
TTCCATTAGCACAGCCATCACTTCCATGCCCTTCTCCCAGGCAGATACCAGGGGCAGACTCAAAGGATTCCTAACAG
CAATAAAAGTAGCTCCTTTAGCTATAGGGTCATTCTTGTCGATCTTCTCAACACTTCTCTTGCCATCCTTCTCAATG
ATGCGCACCGGGGGGTAGCTGAAGCCCACGGCCACCAACTGAGCCTGTTCTCTTTCTTCTTCACTATCCTGGCTGAT
GTCTTGCAGAGGGACATGCTTGGTCTTCCTGGGCTTCTTCTTGGGAGGGATCGGGGGAGGACTGTTGCTCCGCTCCG
GAGACAGGGATGACTGCGAAGTTTCGCTTACCAATACCACCTGGCTCTCGGTAGAAGAACCGGACCCCACACGACGG
TAGGTGTTCCTCTTCGGGGGCAGAGGTGGAGGCGACTGAGATGGGCTGCGGTCTGGCCTTGGAGGCGGATGGCTGGC
AGAGCTCATTCCGCGTTCGGGGGTGTGCTCCCGGTGGCGGTCGCTTGACTGATTTCCTCCGCGGCTGGCCATTGTGT
TCTCCTAGGCAGAGAAACAACAGACATGGAAACTCAGCCATCACTGCCAACATCGCTGCAAGCACCATCACACCTCG
CCCCCAGCAGCGACGAGGAGGAGAGCTTAACCACCCCACCACCCAGTCCCGCTACCACCACCTCTACCCTCGATGAT
GAGGAGGAGGTCGACGCAGCCCAGGAGATGCAGGCGCAGGATAATGTGAAAACGGAAGAGATTGAGGCAGATGTCGA
GCAGGACCCGGGCTATGTGACGCCGGCGGAGCACCAGGAGGAGCTGAAACGCTTTCTAGACAGAGAGGATGACGACC
GCCCAGAGCATCAAGCAGATGGCGTTTACCAGGAGGCTGGGATCAGGGATCATGTCGCCGACTACCTCACCGGCCTT
GGTGGGGAGGACGTGCTCCTCAAACATCTAGCAAGGCAGTCGATCATAGTTAAAGACGCATTGCTCGATCTCACTGA
AGTGCCCATCAGTGTGGAAGAGCTTAGCCGCGCCTACGAGCTGAACCTCTTTTCGCCTCAGGTACCCCCCAAGCGGC
AGCCAAACGGCACCTGCGAGGCCAACCCTCGACTCAACTTCTATCCAGCTTTTACTATCCCCGAAGTGTTGGCCACC
TACCACATCTTTTTCAAGAACCAAAAGATTCCAGTCTCCTGCCGCGCCAACCGCACCCGCGCCGATGCCCTGCTCAA
CTTGGGTCCGGGAGCTCGCTTACCTGATATAGCTTCCTTGGAAGAGGTTCCAAAGATCTTTGAGGGTCTGGGAAGTG
ATGAGACACGGGCCGCAAATGCTCTGCAACAGGGAGAGAATGACATGGATGAACACCACAGCGCTCTGGTGGAACTG
GAGGGTGACAATGCCCGGATTGCAGTGCTCAAGCGCAGTATCGTGGTCACCCATTTTGCCTACCCCGCTGTTAACCT
GCCCCCCAAAGTTATGAGCGCTGTCATGGACCATCTGCTCATCAAACGAGCAAGACCTCTTTCAGAAAACCAGAACA
TGCAGGATCCAGACGCCTCGGACGAGGGCAAGCCGGTAGTCAGTGACGAGCAGCTATCTCGCTGGCTGGGTACCAAC
TCCCCCCGAGATTTGGAAGAGAGGCGCAAGCTTATGATGGCTGTAGTGCTAGTAACTGTGGAGCTGGAGTGTCTGCG
CCGCTTTTTCACCGACCCTGAGACCCTGCGCAAGCTAGAGGAGAACCTGCACTACACCTTTAGACATGGCTTCGTGC
GGCAGGCATGCAAGATCTCCAACGTGGAGCTTACCAACCTGGTTTCTTACATGGGCATTTTGCATGAGAACCGACTA
GGGCAGAGCGTCCTGCACACCACCCTTAAAGGGGAGGCCCGCCGTGACTACATCCGAGACTGTGTCTACCTCTACCT
CTGCCATACCTGGCAAACTGGTATGGGTGTGTGGCAACAGTGTTTGGAAGAGCAGAACCTAAAAGAGCTGGACAAGC
TCTTGCAGAGATCCCTCAAAGCCCTGTGGACAGGTTTTGATGAGCGCACCGTCGCCTCGGACCTGGCAGACATCATC
TTCCCCGAGCGTCTCAGGGTTACTCTGCGAAACGGCCTGCCAGACTTTATGAGCCAGAGCATGCTTAACAACTTTCG
CTCTTTCATCCTGGAACGCTCCGGTATCCTGCCTGCCACCTGCTGTGCGCTGCCCTCCGACTTTGTGCCTCTCACCT
ACCGCGAGTGCCCACCGCCGCTATGGAGCCACTGCTACCTGTTCCGCCTGGCCAACTACCTCTCCTACCACTCGGAT
GTTATAGAGGATGTGAGCGGAGACGGCCTGCTGGAATGCCACTGCCGCTGCAATCTTTGCACACCCCACCGCTCCCT
TGCCTGCAACCCCCAGTTGCTGAGCGAGACCCAGATTATCGGCACCTTCGAGCTGCAGGGTCCCAGAAGTAAAGGCG
AGGGGTCTTCTCCGGGGCAGAGTTTGAAACTGACACCGGGGCTGTGGACCTCCGCCTACCTGCGCAAGTTTCACCCC
GAGGACTACCATCCCTATGAGATCAGGTTCTATGAGGACCAATCACATCCTCCCAAAGTCGAGCTCTCAGCCTGCGT
CATCACCCAGGGAGCAATTCTGGCCCAATTGCAAGCCATCCAAAAATCTCGCCAAGAATTTCTGCTAAAAAAGGGAA
ACGGGGTCTACCTTGACCCTCAGACCGGTGAGGAGCTCAACACAAGGTTCCCCCAGGATGTCCCATCGCCGAGGAAG
CAAGAAGTTGAAGGTGCAGCTGTCGCCCCCAGAGGATATGAAGGAAGACTGGGACAGTCAGGCAGAGGAGGAGATGG
AAGATTGGGACAGCCAGGCAGAGGAGGTGGACAGCCTGGAGGAAGACAGTTTGGAGGAGGAAGACGAGGAGGCAGAG
GAGGTGGAAGAAGCAACCGCCGCCAAACAGTTGTCATCGGCGGCGGAGACAAGCAAGTCCCCAGACAGCAGCACGGC
TACCATCTCCGCTCCGGGTCGGGGGGCCCAGCGGCGGCCCAACAGTAGATGGGACGAGACCGGGCGATTCCCAAACC
CGACCACCGCTTCCAAGACCGGTAAGAAGGAGCGACAGGGATACAAGTCCTGGCGTGGACATAAAAACGCTATCATC
TCCTGCTTGCATGAATGCGGGGGCAACATATCCTTCACCCGGCGATACCTGCTCTTCCACCACGGTGTAAACTTCCC
CCGCAATATCTTGCATTACTACCGTCACCTCCACAGCCCCTACTGCAGTCAGCAAGTCCCGGCAACCCCGACAGAAA
AATACAGCAGCGACAACGGTGACCAGAAAACCAGCAGTTAGAAAATCCACAACAAGTGCACCAGGAGGAGGACTGAG
GATCACAGCGAACGAGCCAGCGCAGACCAGAGAGCTGAGGAACCGGATCTTTCCAACCCTCTATGCCATTTTCCAGC
AGAGTCGGGGGCAAGAGCAGGAACTGAAAGTAAAAAACCGATCTCTGCGCTCGCTCACCAGAAGTTGTTTGTATCAC
AAGAGCGAAGACCAACTTCAGCGCACTCTCGAGGACGCCGAGGCTCTCTTCAACAAGTACTGCGCGCTGACTCTTAA
AGAGTAGCCCTTGCCCGCGCTCATTTTGAAAACGGCGGGAATCACGTCACCCTTGGCACCTGTCCTTTGCCCTTGTC
ATGAGTAAAGAGATTCCCACGCCTTACATGTGGAGCTATCAGCCCCAAATGGGGTTGGCAGCAGGCGCTTCCCAGGA
CTACTCCACCCGCATGAATTGGCTTAGCGCCGGGCCCTCAATGATATCACGGGTTAATGATATACGAGCTTATCGAA
ACCAGTTACTCCTAGAACAGTCAGCTCTCACCACCACACCCCGTCAACACCTTAATCCCCGAAATTGGCCCGCCACC
CTGGTGTACCAGGAAAATCCCGCTCCCACCACCGTACTACTTCCTCGAGACGCCCAGGCCGAAGTTCAGATGACTAA
CGCAGGTGTACAGCTGGCGGGCGGTTCCGCCCTATGTCGTCACCGACCTCAACAGAGTATAAAACGCCTGGTGATTA
GAGGCCGAGGTATCCAGCTCAACGACGAGTCGGTTAGCTCTTCGCTTGGTCTGCGACCAGACGGAGTCTTCCAAATC
GCCGGCTGTGGGAGATCTTCCTTCACTCCTCGTCAGGCTGTGCTGACTTTGGAGAGTTCGTCCTCGCAGCCCCGCTC
GGGCGGCATTGGAACTCTCCAGTTTGTGGAGGAGTTTACTCCCTCTGTCTACTTCAACCCCTTCTCCGGCTCTCCTG
GCCAGTACCCGGACGAGTTCATACCAAACTTCGACGCAATCAGCGAGTCAGTGGATGGCTATGATTGATGTCTAATG
GTGGTGCGGCTGAGCTAGCTCGACTGCGACACCTAGACCACTGCCGCCGCTTTCGCTGCTTCGCCCGGGAACTCACC
GAGTTCATCTACTTCGAACTCTCCGAGGAGCACCCTCAGGGTCCGGCCCACGGAGTGCGGATTACCATCGAAGGGGG
AATAGACTCTCGCCTGCATCGCATCTTCTCCCAGCGGCCCGTGCTAATTGAACGCGACCAGGGAAATACAACCATCT
CCATCTACTGCATCTGTAACCACCCCGGATTGCATGAAAGCCTTTGCTGTCTTGTTTGTGCTGAGTTTAATAAAAAC
TGAGTTAAGACCCTCCTACGGACTACCGCTTCTTCAATCAGGACTTTACAACACCAACCAGATCTTCCAGAAGACCC
AGACCCTTCCTCCTTTCATCCAGGACTCTAACTCTACCTTACCAGCACCCTCCACTACTAACCTTCCCGAAACAAAC
AAGCTTGCATCTCATCTGCAACACCGCCTTTCACGAAGCCTTCTTTCTGCCAATACTACCACTCCCAAAACCGGAGG
TGAGCTCCGCGGTCTTCCTACTGACGACCCCTGGGTGGTAGCGGGTTTTGTAACGTTAGGAGTAGTTGCGGGTGGGC
TTGTGCTGATCCTTTGCTACCTATACACACCTTGCTGTGCATATTTAGTCATATTGTGCTGTTGGTTTAAGAAATGG
GGGCCATACTAGTCGTGCTTGCTTTACTTTCGCTTTTGGGTCTGGGCTCTGCTAATCTCAATCCTCTCGATCACGAT
CCATGTTTAGACTTCGACCCAGAAAACTGCACACTTACTTTTGCACCCGACACAAGCCGTCTCTGTGGAGTTCTTAT
TAAGTGCGGATGGGACTGCAGGTCCGTTGAAATTACACATAATAACAAAACATGGAACAATACCTTATCCACCACAT
GGGAGCCAGGAGTTCCCCAGTGGTATACTGTCTCTGTCCGAGGTCCTGACGGTTCCATCCGCATTAGTAACAACACT
TTCATTTTTTCTGAAATGTGCGATCTGGCCATGTTCATGAGCAGACAGTATGACCTATGGCCTCCCAGCAAAGAGAA
CATTGTGGCATTTTCCATTGCTTATTGCTTGGTAACATGCATCATCACTGCTATCATTTGTGTGTGCATACACTTGC
TTATAGTTATTCGCCCTAGACAAAGCAATGAGGAAAAAGAGAAAATGCCTTAACCTTTTTCCTCATACCTTTTCTTT
ACAGCATGGCTTCTGTTACAGCTCTAATTATTGCCAGCATTGTCACTGTCGCTCACGGGCAAACAATTGTCCATATT
ACCTTAGGACATAATCACACTCTTGTAGGGCCCCCAATTACTTCAGAGGTTATTTGGACCAAACTTGGAAGTGTTGA
TTATTTTGATATAATTTGCAACAAAACTAAACCAATATTTGTAATCTGTAACAGACAAAATCTCACGTTAATTAATG
TTAGCAAAATTTATAACGGTTACTATTATGGTTATGACAGATCCAGTAGTCAATATAAAAATTACTTAGTTCGCATA
ACTCAGCCCAAATTAACAGTGCCAACTATGACAATAATTAAAATGGCTAATAAAGCATTAGAAAATTTTACATCACC
AACAACACCCAATGAAAAAAACATTCCAAATTCAATGATTGCAATTATTGCGGCGGTGGCATTGGGAATGGCACTAA
TAATAATATGCATGCTCCTATATGCTTGTTACTATAAAAAGTTTCAACATAAACAGGATCCACTACTAAATTTTAAC
ATTTAATTTTTTATACAGATGATTTCCACTACAATTTTTATCATTACTAGCCTTGCAGCTGTAACTTATGGCCGTTC
ACACCTAACTGTACCTGTTGGCTCAACATGTACACTACAAGGACCCCAAGAAGGCTATGTCACTTGGTGGAGAATAT
ATGATAATGGAGGGTTCGCTAGACCATGTGATCAGCCTGGTACAAAATTTTCATGCAACGGAAGAGACTTGACCATT
ATTAACATAACATCAAATGAGCAAGGCTTCTATTATGGAACCAACTATAAAAATAGTTTAGATTACAACATTATTGT
AGTGCCAGCCACCACTTCTGCTCCCCGCAAATCCACTTTCTCTAGCAGCAGTGCCAAAGCAAGCACAATTCCTAAAA
CAGCTTCTGCTATGTTAAAGCTTCCAAAAATCGCTTTAAGTAATTCCACAGCCGCTCCCAATACAATTCCTAAATCA
ACAATTGGCATCATTACTGCCGTGGTAGTGGGATTAATGATTATATTTTTGTGTATAATGTACTACGCCTGCTGCTA
TAGAAAACATGAACAAAAAGGTGATGCATTACTAAATTTTGATATTTAATTTTTTATAGAATTATGATATTGTTTCA
ATCAAATACCACTACCTCCTATGCATACACAAACATTCAGCCTAAATACGCTATGCAACTAGAAATCACAATACTAA
TTGTAATTGGAATTCTTATACTATCTGTTATTCTTTATTTTATATTCTGCCGTCAAATACCCAATGTTCATAGAAAT
TCTAAAAGACGTCCCATCTATTCTCCTATGATTAGTCGTCCCCATATGGCTCTGAATGAAATCTAAGATCTTTTTTT
TTTTCTCTTACAGTATGGTGAACATCAATCATGATCCCTAGAAATTTCTTCTTCACCATACTCATCTGTGCTTTTAA
TGTCTGTGCTACTTTCACAGCAGTAGCCACTGCAAGCCCAGACTGTATAGGACCATTTGCTTCCTATGCACTTTTTG
CCTTCGTTACTTGCATCTGCGTGTGTAGCATAGTCTGCCTGGTTATTAATTTTTTCCAACTGGTAGACTGGATCTTT
GTGCGAATTGCCTACCTACGTCACCATCCCGAATACCGCAATCAAAATGTTGCGGCACTTCTTAGGCTTATTTAAAA
CCATGCAGGCTATGCTACCAGTCATTTTAATTTTGCTACTACCCTGCATTCCCCTAGCTTCCACCGCCACTCGCGCT
ACACCTGAACAACTTAGAAAATGCAAATTTCAACAACCATGGTCATTTCTTGATTGCTACCATGAAAAATCTGATTT
TCCCACATACTGGATAGTGATTGTTGGAATAATTAACATACTTTCATGTACCTTTTTCTCAATCACAATATACCCCA
CATTTAATTTTGGGTGGAATTCTCCCAATGCACTGGGTTACCCACAAGAACCAGATGAACATATTCCACTACAACAC
ATACAACAACCACTAGCACTGGTACAGTATGAAAATGAGCCACAACCTTCACTGCCCCCTGCCATTAGTTACTTCAA
CCTAACCGGCGGAGATGACTGACCCAATCGCCACATCATCCACCGCTGCCAAGGAGCTGCTGGACATGGACGGACGT
GCCTCAGAACAGCGACTCATCCAACTACGCATTCGTCAGCAGCAGGAACGAGCAGTAAAAGAGCTAAGGGATGCCAT
TGGGATTCACCAGTGCAAAAAAGGCATATTCTGCTTAGTAAAACAATCCAAAATCTCCTACGAGATCACCGCTACTG
ACCATCGTCTCTCATACGAGCTCGGTCCGCAGCGACAAAAATTCACCTGCATGGTGGGAATCAACCCCATAGTTATC
ACCCAGCAGTCTGGAGATACTAAGGGTTGTATCCAGTGTTCCTGTGATTCCACCGAGTGCATCTACACACTGCTGAA
GACCCTCTGCGGCCTTCGAGACCTCCTACCCATGAACTAATCATTGCCCCTACCTTACCCAATCAAAATATTAATAA
AGACACTTACTTGAAATCAGCAATACAGTCTTTGTCAAAACTTTCTACCAGCAGCACCTCACCCTCTTCCCAACTCT
GGTACTCTAAACGTCGGAGGGTGGCATACTTTCTCCACACTTTGAAAGGGATGTCAAATTTTATTTCCTCTTCTTTG
CCCACAATCTTCATTTCTTTATCCCCAGATGGCCAAGCGAGCTCGGCTAAGCACTTCCTTCAACCCGGTGTACCCTT
ATGAAGATGAAAGCAGCTCACAACACCCATTTATAAATCCTGGTTTCATTTCCCCTGACGGGTTCACACAAAGTCCA
AACGGGGTTTTAAGTCTTAAATGTGTTAATCCACTTACCACTGCAAGCGGCTCCCTCCAACTTAAAGTGGGAAGTGG
TCTTACAGTAGACACTACTGATGGATCCTTAGAAGAAAACATCAAAGTTAACACCCCCCTAACAAAGTCAAACCATT
CTATAAATTTACCAATAGGAAACGGTTTGCAAATAGAACAAAACAAACTTTGCAGTAAACTCGGAAATGGTCTTACA
TTTGACTCTTCCAATTCTATTGCACTGAAAAATAACACTTTATGGACAGGTCCAAAACCAGAAGCCAACTGCATAAT
TGAATACGGGAAACAAAACCCAGATAGCAAACTAACTTTAATCCTTGTAAAAAATGGAGGAATTGTTAATGGATATG
TAACGCTAATGGGAGCCTCAGACTACGTTAACACCTTATTTAAAAACAAAAATGTCTCCATTAATGTAGAACTATAC
TTTGATGCCACTGGTCATATATTACCAGACTCATCTTCTCTTAAAACAGATCTAGAACTAAAATACAAGCAAACCGC
TGACTTTAGTGCAAGAGGTTTTATGCCAAGTACTACAGCGTATCCATTTGTCCTTCCTAATGCGGGAACACATAATG
AAAATTATATTTTTGGTCAATGCTACTACAAAGCAAGCGATGGTGCCCTTTTTCCGTTGGAAGTTACTGTTATGCTT
AATAAACGCCTGCCAGATAGTCGCACATCCTATGTTATGACTTTTTTATGGTCCTTGAATGCTGGTCTAGCTCCAGA
AACTACTCAGGCAACCCTCATAACCTCCCCATTTACCTTTTCCTATATTAGAGAAGATGACTGACAACAAAAATAAA
GTTCAACATTTTTTATTGAAATTCCTTTTACAGTATTCGAGTAGTTATTTTGCCTCCCCCTTCCCATTTAACAGAAT
ACACCAATCTCTCCCCACGCACAGCTTTAAACATTTGGATACCATTAGAGATAGACATAGTTTTAGATTCCACATTC
CAAACAGTTTCAGAGCGAGCCAATCTGGGGTCAGTAATACATAAAAATGCATCGGGATAGTCTTTTAAAGCGCTTTC
ACAGTCCAACTGTTGCGGATGCGACTCCGGAGTCTGAATCACGGTCATCTGGAAGAAGAACGATGGGAATCATAATC
CGAAAACGGAATCGGGCGATTGTGTCTCATCAAACCCACAAGCAACCGCTGTCTGCGTCGCTCCGTGCGACTGCTGT
TTATGGGATCGGGGTCCGCAGTGTCCTGAAGCATGATTTTAATAGCCCTTAACATTAACTTTCTGGTGCGATGCGCG
CAGCAACGCATTCTGATTTCACTTAGATTACTACAGTAGGTACAGCACATTATCACAATATTGTTTAATAAACCATA
ATTAAAAGCGCTCCAGCCAAAACTCATATCTGATATAATCGCCCCTGCATGACCATCATACCAAAGTTTAATATAAA
TTAAATGTCGTTCCCTCAAAAACACACTACCCACATACATGATCTCTTTTGGCATGTGCATATTAACAATTTGTCTG
TACCATGGACAACGTTGGTTAATCATGCAACCCAATATAACCTTCCGGAACCACACTGCCAACACCGCTCCCCCAGC
CATGCATTGAAGTGAACCCTGCTGATTACAATGACAATGAAGAACCCAATTCTCTCGACCATGAATCACTTGAGACT
GAAAAATATCTATAGTAGCACAACAAAGACATAAATGCATGCATCTTCTCATAATTTTTAACTCCTCTGGATTTAAA
AACATATCCCAAGGAATGGGAAACTCTTGCAGAACAGTAAAGCTGGCAGAACAAGGAAGACCACGAACACAACTTAC
ACTATGCATAGTCATAGTATCACAATCTGGCAACAGCGGGTGGTCTTCAGTCATAGAAGCTCGGGTTTCATTTTCCT
CACATCGTGGTAATTGGGCTCTGGTGTAAGGGTGATGTCTGGCGCATGATGTGGAGCGTGCGCGCAACCTTGTCATA
ATGGAGTTGCTTCCTGACATTCTCGTATTTTGTATAGCAAAACGCTGCCCTGGCACAACACACTCTTCTTCGTCTTC
TATCCTGCCGCTTAGTGTGTTCCGTCTGATAATTCAAGTACAGCCACACTCTTAAGTTGGTCAAAAGAATGCTGGCT
TCAGTTGTAATCAAAACTCCATCATATTTAATTGTTCTAAGGAAATCATCCACGGTAGCATATGCAAATCCCAACCA
AGCAATGCAACTGGATTGCGTTTCAAGCAGCAGAGGAGAGGGAAGAGACGGAAGAATCATGTTAATTTTTATTCCAA
ACGATCTCGCAGTACTTCAAATTGTAGATCGCGCAGATGGCATCTATCGCCCCCACTGTGTTGGTGAAAAAGCACAG
CTAAATCAAAAGAAATGCGATTTTCAAGGTGCTCAACGGTGGCTTCCAACAAAGCCTCCACGCGCACATCCAAAAAC
AAAAGAATACCAAAAGAAGGAGCATTTTCTAACTCCTCAAACATCATATTACATTCCTGCACCATTCCCAGATAATT
TTCAGCTTTCCAGCCTTGAATTATTCGTGTCAGTTCTTGTGGTAAATCCAAACCACACATTACAAACAGGTCCCGGA
GGGCGCCCTCCACCACCATTCTTAAACACACCCTCATAATGACAAAATATCTTGCTCCTGTGTCACCTGTAGCAAAT
TAAGAATGGCATCATCAATTGACATGCCCTTGGCTCTAAGTTCTTCTCTAAGTTCTAGTTGTAGATACTCTCTCATA
TTATCACCAAACTGCTTAGCCAGAAGCCCCCCGGGAACAATAGCAGGGGACGCTACAGTGCAGTACAAGCGCAGACC
TCCCCAATTGGCTCCAGCAAAAACAAGATTAGAATAAGCATACTGGGAACCACCAGTAATATCATCAAAGTTGCTGG
AAATATAATCAGGCAGAGTTTCTTGTAAAAATTGAATAAAAGAAAAATTTTCCAAAGAAACATTCAAAACCGTTGGG
ATGCAAATACAATAGGTTACCGCGCTGCGCTCCAACATTGTTAGTTTTGAATTAGTCTGCAAAATAAAAGAAACAAG
CGTCATATCATAGTAGCCTGTCGAACAGGTGGAAAAATCAGTCTTTCCATCACAAGACAAGCCACAGGGTCTCCAGC
TCGACCCTCGTAAAACCTGTCATTGTGATTAAACAACAGCACCGAAAGTTCCTCGCGGTGGCCAGCATGAATAATTC
TTGATGAAGCATACAATCCAGACATGTTAGCATCAGTTAAAGAGAAAAAACAGCCAACATAGCCTCTGGGTATAATT
ATGCTTAATTTTAAGTATAGCAAAGCCACCCCTCGCGGATACAAAGTAAAAGGCACAGGAGAATAAAAAATATAATT
ATTTCTCTGCTGCTGTTCAGGCAACGTTGCTCCCGGTCCCTCTAAATAGACATACAAAGCCTCATCAGCCATGGCTT
ACCAGGCAAAGTACAGCGGGCGCACAAAGCACAAGCTCTAAAGAAGCTCTAAAAACACTCTCCAACCTCTCCACAAT
ATATACACAAGCCCTAAACTGACGTAATGGGAGTAAAGTGAAAAAAAAATACCGCCAAGCCCAACACACACCCCGAA
ACTGCGTCAGCAGGAAAAAGTACAGTTTCACTTCCGCATTCCCAACAAGCGTAACTTCCTCTTTCTCATGGTACGTC
ACATCCGATTAACTTGCAACGTCATTTTCCCACGGTCGCGCCGCCCCTTTTAGCCGTTAACCCCGCAGCCAATCACC
ACACAGCGCGCACTTTTTTAAATTACCTCATTTACATGTTGGCACCATTCCATCTATAAGGTATATTATATAGATAG

GenBank Accession No. YP 002213796 (SEQ ID NO: 214)

MAKRARLSTSFNPVYPYEDESSSQHPFINPGFISPDGFTQSPNGVLSL
KCVNPLTTASGSLQLKVGSGLTVDTTDGSLEENIKVNTPLTKSNHSIN
LPIGNGLQIEQNKLCSKLGNGLTFDSSNSIALKNNTLWTGPKPEANCI
IEYGKQNPDSKLTLILVKNGGIVNGYVTLMGASDYVNTLFKNKNVSIN
VELYFDATGHILPDSSSLKTDLELKYKQTADFSARGFMPSTTAYPFVL
PNAGTHNENYIFGQCYYKASDGALFPLEVTVMLNKRLPDSRTSYVMTF
LWSLNAGLAPETTQATLITSPFTFSYIREDD

GenBank Accession No. YP_002213774 (SEQ ID NO: 215)

MRRRAVLGGAVVYPEGPPPSYESVMQQQAAMIQPPLEAPFVPPRYLAP
TEGRNSIRYSELSPLYDTTKLYLVDNKSADIASLNYQNDHSNFLTTVV
QNNDFTPTEASTQTINFDERSRWGGQLKTIMHTNMPNVNEYMFSNKFK
ARVMVSRKAPEGVTVNDTYDHKEDILKYEWFEFILPEGNFSATMTIDL
MNNAIIDNYLEIGRQNGVLESDIGVKFDTRNFRLGWDPETKLIMPGVY
TYEAFHPDIVLLPGCGVDFTESRLSNLLGIRKRHPFQEGFKIMYEDLE
GGNIPALLDVTAYEESKKDTTTETTTLAVAEETSEDDDITRGDTYITE
KQKREAAAAEVKKELKIQPLEKDSKSRSYNVLEDKINTAYRSWYLSYN
YGNPEKGIRSWTLLTTSDVTCGAEQVYWSLPDMMQDPVTFRSTRQVNN
YPVVGAELMPVFSKSFYNEQAVYSQQLRQATSLTHVFNRFPENQILIR
PPAPTITTVSENVPALTDHGTLPLRSSIRGVQRVTVTDARRRTCPYVY
KALGIVAPRVLSSRTF

GenBank Accession No. YP_002213779 (SEQ ID NO: 216)

MATPSMMPQWAYMHIAGQDASEYLSPGLVQFARATDTYFSMGNKFRNP
TVAPTHDVTTDRSQRLMLRFVPVDREDNTYSYKVRYTLAVGDNRVLDM
ASTFFDIRGVLDRGPSFKPYSGTAYNSLAPKGAPNTSQWIVTTNGDNA
VTTTTNTFGIASMKGDNITKEGLQIGKDITTTEGEEKPIYADKTYQPE
PQVGEESWTDTDGTNEKFGGRALKPATNMKPCYGSFARPTNIKGGQAK
NRKVKPTTEGGVETEEPDIDMEFFDGRDAVAGALAPEIVLYTENVNLE
TPDSHVVYKPETSNNSHANLGQQAMPNRPNYIGFRDNFVGLMYYNSTG
NMGVLAGQASQLNAVVDLQDRNTELSYQLLLDSLGDRTRYFSMWNQAV
DSYDPDVRIIENHGIEDELPNYCFPLNGIGPGHTYQGIKVKIDDINGW
EKDANVAPANEITIGNNLAMEINIQANLWRSFLYSNVALYLPDVYKYT
PPNITLPTNTNTYEYMNGRVVSPSLVDSYINIGARWSLDPMDNVNPFN
HHRNAGLRYRSMLLGNGRYVPFHIQVPQKFFAVKNLLLLPGSYTYEWN
FRKDVNMVLQSSLGNDLRTDGATISFTSINLYATFFPMAHNTASTLEA
MLRNDTNDQSFNDYLSAANMLYPIPANATNIPISIPSRNWAAFRGWSF
TRLKTKETPSLGSGFDPYFVYSGSIPYLDGTFYLNHTFKKVSIMFDSS
VSWPGNDRLLSPNEFEIKRTVDGEGYNVAQCNMTKDWFLVQMLANYNI
GYQGFYIPEGYKDRMYSFFRNFQPMSRQVVDEVNYTDYKAVTLPYQHN
NSGFVGYLAPTMRQGEPYPANYPYPLIGTTAVKSVTQKKFLCDRTMWR
IPFSSNFMSMGALTDLGQNMLYANSAHALDMTFEVDPMDEPTLLYLLF
EVFDVVRVHQPHRGVIEAVYLRTPFSAGNATT

GenBank Accession No. AC_000018 (SEQ ID NO: 200)

CTCTCTATTTAATATACCTTATAGATGGAATGGTGCCAATATGTAAATGAGGTAATTTAAAAAAGTGCGCGCTGTGT
GGTGATTGGCTGTGGGGTTAACGGCTAAAATGGGCGGGGCGGCCGTGGGAAAATGACGTGACTTATGTGGGAGGAGC
TATGTTGCAAGTTATTGCGGTAAATGTGACGTAAAACGAGGTGTGGTTTGAACACGGAAGTAGACAGTTTTCCCACG
CTTACTGACAGGATATGAGGTAGTTTTGGGCGGATGCAAGTAAAAATTCTCCATTTTCGCGCGAAAACTGAATGAGG
AAGTGAATTTCTGAGTCATTTCGCGGTTATGACAGGGTGGAGTATTTGCCGAGGGCCGAGTAGACTTTGACCGTTTA
CGTGGAGGTTTCGATTACCGTGTTTTTCACCTAAATTTCCGCGTACGGTGTCAAAGTCCTGTGTTTTTACGTAGGTG
TCAGCTGATCGCTAGGGTATTTAAACCTGACGAGTTCCGTCAAGAGGCCACTCTTGAGTGCCAGCGAGAAGAGTTTT
CTCCTTCGCGCCGCAAGTCAGTTCTGCGCTTTGAAAATGAGACACCTGCGTTTCCTGCCACAGGAGATTATCTTCAG
TGAGACCGGGATCGAAATACTGGAGTTTGTGGTAAATACCCTAATGGGAGACGACCCGGAACCGCCAGTGCAGCCTT
TCGATCCACCTACGCTGCACGATCTGTATGATTTAGAGGTAGACGGGCCTCAGGATCCCAATGAGGAAGCTGTGAAT
GGGTTTTTTACTGATTCTATGCTGCTAGCTGCCGATGAAGGATTGGACATAAACCCTCCTCCTGAGACCCTTGTTAC
CCCAGGGGTGGTTGTGGAAAGCGGCAGAGGTGGGAAAAAATTGCCTGATCTGGGAGCAGCTGAAATGGACTTGCGTT
GTTATGAAGAGGGTTTTCCTCCGAGTGATGATGAAGATGGGGAAACTGAGCAGTCCATCCATACCGCAGTGAATGAG
GGAGTAAAAGCTGCCAGCGATGTTTTTAAGTTGGACTGTCCGGAGCTGCCTGGACATGGCTGTAAGTCTTGTGAATT
TCACAGGAATAACACTGGAATGAAAGAACTATTGTGCTCGCTTTGCTATATGAGAATGCACTGCCACTTTATTTACA
GTAAGTGTATTTAAGTGAAATTTAAAGGAATAGTGTAGCTGTTTAATAACTGTTGAATGGTAGATTTATGTTTTTTA
CTTGTGATTTTTTGTAGGTCCTGTGTCTGATGATGAGTCACCTTCTCCTGATTCAACTACCTCACCTCCTGAAATTC
AGGCGCCCGCACCTGCAAACGTATGCAAGCCCATTCCTGTAAAGCCTAAGCCTGGGAAACGCCCTGCTGTGGATAAG
CTTGAGGACTTGTTGGAGGGTGGGGATGGACCTTTGGACCTTAGTACCCGGAAACTGCCAAGGCAATAAGTGCCCTG
CAGCTGTGTTTATTTAATGTGACGTCATGTAATAAAATTATGTCAGCTGCTGAGTGTTTTATTACTTCTTGGGTGGG
GACTTGGATATATAAGTAGGAGCAGATCTGTGTGGTTAGCTCACAGCAACCTGCTGCCATCCATGGAGGTTTGGGCT
ATCTTGGAAGACCTCAGACAGACTAGGCTACTGCTAGAAAACGCCTCGGACGGAGTCTCTGGCCTTTGGAGATTCTG
GTTCGGTGGTGATCTAGCTAGGCTAGTGTTTAGGATAAAACAGGACTACAGCGTAGAATTTGAAAAGTTATTGGACG
ACAGTCCAGGACTTTTTGAAGCTCTTAACTTGGGTCATCAGGCTCATTTTAAGGAGAAGGTTTTATCAGTTTTAGAT
TTTTCTACTCCTGGTAGAACTGCTGCTGCTGTAGCTTTTCTTACTTTTATATTGGATAAATGGATCCGCCAAACTCA
CTTCAGCAAGGGATACGTTTTGGATTTCATAGCAGCAGCTTTGTGGAGAACATGGAAGGCTCGCAGGATGAGGACAA
TCTTAGATTACTGGCCAGTGCAGCCTCTAGGAGTAGCAGGGATACTGAGACACCCACCGACCATGCCAGCGGTTCTG
CAGGAGGAGCAGCAGGAGGACAATCCGAGAGCCGGCCTGGACCCTCCGGTGGAGGAGTAGCTGACCTGTTTCCTGAA
CTGCGACGGGTGCTTACTAGGTCTACGACCAGTGGACAGAACAGAGGCATTAAGAGGGAGAGGAATCCTAGTGGGAA
TAATTCAAGAACCGAGTTGGCTTTAAGTTTAATGAGCCGCAGGCGTCCTGAAACTGTTTGGTGGCATGAGGTTCAGA
GCGAAGGCAGGGATGAAGTTTCAATATTGCAGGAGAAATATTCACTAGAACAACTTAAGACCTGTTGGTTGGAACCT
GAGGATGATTGGGAGGTGGCCATTAGGAATTATGCTAAGATATCTCTGAGGCCTGATAAACAATATAGAATTACTAA
GAAGATTAATATTAGAAATGCATGCTACATATCAGGGAATGGGGCAGAGGTTATAATAGATACACAAGATAAAGCAG
CTTTTAGATGTTGTATGATGGGTATGTGGCCAGGGGTTGTCGGCATGGAAGCAGTAACACTTATGAATATTAGGTTT
AGAGGGGATGGGTATAATGGCATTGTATTTATGGCTAACACTAAGCTGATTCTACATGGTTGTAGCTTTTTTGGGTT
TAATAATACGTGTGTAGAAGCTTGGGGGCAAGTTAGTGTGAGGGGTTGTAGTTTTTATGCATGCTGGATTGCAACAT
CAGGTAGGGTCAAGAGTCAGTTGTCTGTGAAGAAATGCATGTTTGAGAGATGTAATCTTGGCATACTGAATGAAGGT
GAAGCAAGGATCCGCCACTGCGCAGCTACAGAAACTGGCTGCTTCATTCTAATAAAGGGAAATGCCAGTGTGAAGCA
TAATATGATCTGTGGACATTCGGATGAGAGGCCTTATCAGATGCTGACCTGCGCTGGTGGACATTGCAATATTCTTG
CTACTGTGCATATCGTTTCACATGCACGCAAGAAATGGCCTGTATTTGAACATAATGTGATTACCAAGTGCACCATG
CACATAGGTGGTCGCAGGGGAATGTTTATGCCTTACCAGTGTAACATGAATCATGTGAAGGTAATGTTGGAACCAGA
TGCCTTTTCCAGAGTGAGCTTAACAGGAATCTTTGATATGAATATTCAACTATGGAAGATCCTGAGATATGATGACA
CTAAACCGAGGGTGCGCGCATGCGAATGCGGAGGCAAGCATGCTAGATTCCAGCCGGTGTGCGTGGATGTGACTGAA
GACCTGAGACCCGATCATTTGGTGCTTGCCTGCACTGGAGCGGAGTTCGGTTCCAGTGGTGAAGAAACTGACTAAAG
TGAGTAGTGGGGGCAAGATGTGGATGGGGACTTTCAGGTTGGTAAGGTGGACAGATTGGGTAAATTTTGTTAATTTC
TGTCTTGCAGCTGCCATGAGTGGAAGCGCTTCTTTTGAGGGGGGAGTATTTAGCCCTTATCTGACGGGCAGGCTCCC
ACCATGGGCAGGAGTTCGTCAGAATGTCATGGGATCCACTGTGGATGGGAGACCCGTCCAGCCCGCCAATTCCTCAA
CGCTGACCTATGCCACTTTGAGTTCGTCACCATTGGATGCAGCTGCAGCCGCCGCCGCTACTGCTGCCGCCAACACT
ATCCTTGGAATGGGCTATTACGGAAGCATCGTTGCCAATTCCAGTTCCTCTAATAACCCTTCAACCCTGGCTGAGGA
CAAGCTACTTGTTCTGTTGGCTCAGCTCGAGGCCTTAACCCAACGCTTAGGCGAACTGTCTAAGCAGGTGGCCCAGT
TGCGTGAGCAAACTGAGTCTGCTGTTGCTACAGCAAAGTCTAAATAAAGATCTCAAATCAATAAATAAAGAAATACT
TGTTATAAAAACAAATGAATGTTTATTTGATTTTTCGCGCGCGGTATGCCCTGGACCATCGGTCTCGATCATTGAGA
ACTCGGTGGATCTTTTCCAGTACCCTGTAAAGGTGGGATTGAATGTTTAGATACATAGGCATTAGTCCGTCTCGGGG
GTGGAGATAGCTCCATTGAAGAGCCTCTTGCTCCGGGGTAGTGTTATAAATCACCCAGTCATAGCAAGGTCGGAGTG
CATGGTGTTGCACAATATCTTTTAGGAGCAGACTAATTGCAACGGGGAGGCCCTTAGTGTAGGTGTTTACAAATCTA
TTGAGCTGGGACGGGTACATCCGGGGTGAAATTATATGCATTTTGGACTGGATCTTGAGGTTGGCAATGTTGCCGCC
TAGATCCCGTCTCGGGTTCATATTGTGCAGGACCACTAAGACAGTGTATCCGGTGCACTTGGGAAATTTATCATGCA
GCTTAGAGGGAAAAGCATGAAAAAATTTGGAGACGCCTTTGTGACCCCCCAGATTCTCCATGCACTCATCCATAATG
ATAGCGATGGGGCCGTGGGCAGCGGCACGGGCGAACACGTTCCGGGGGTCTGAAACATCATAGTTATGCTCCTGAGT
CAGGTCATCATAAGCCATTTTAATAAACTTTGGGCGGAGGGTGCCAGATTGGGGGATGAAAGTTCCCTCTGGCCCGG
GAGCATAGTTCCCCTCACATATTTGCATTTCCCAGGCTTTCAGTTCAGAGGGGGGGATCATGTCCACCTGCGGGGCT
ATAAAAAATACCGTTTCTGGAGCCGGGGTGATTAACTGGGATGAGAGCAAATTCCTAAGCAGCTGAGACTTGCCGCA
CCCAGTGGGACCGTAAATGACCCCAATTACGGGTTGCAGATGGTAGTTTAGGGAGCGACAGCTGCCGTCCTCCCGGA
GTAGGGGGGCCACTTCGTTCATCATTTCCCTTACATGGATATTTTCCCGCACCAAGTCCGTTAGGAGGCGCTCTCCC
CCAAGGGATAGAAGCTCCTGGAGCGAGGAGAAGTTTTTCAGCGGCTTCAGCCCGTCAGCCATGGGCATTTTGGAAAG
AGTCTGTTGCAAGAGCTCGAGCCGGTCCCAGAGCTCGGTGATGTGCTCTATGGCATCTCGATCCAGCAGACCTCCTC
GTTTCGCGGGTTGGGACGGCTCCTGGAGTAGGGAATCAGACGATGGGCGTCCAGCGCTGCCAGGGTCCGATCCTTCC
ATGGTCGCAGCGTCCGAGTCAGGGTTGTTTCCGTCACGGTGAATGGGTGCGCGCCTGGTTGGGCGCTTGCGAGGGTG
CGCTTCAGACTCATCCTGCTGGTCGAGAACCGCTGCCGATCGGCGCCCTGCATGTCGGCCAGGTAGCAGTTTACCAT
GAGTTCGTAGTTGAGCGCCTCGGCCGCGTGGCCTTTGGCACGGAGCTTACCTTTGGAAGTTTTATGGCAGGCGGGGC
AGTAGATACATTTGAGGGCATACAGCTTGGGCGCGAGGAAAATGGATTCGGGGGAGTATGCATCCGCACCGCAGGAG
GCGCAGACGGTTTCGCACTCCACGAGCCAGGTCAGATCCGGCTCATCGGGGTCAAAAACAAGTTTTCCGCCATGTTT
TTTGATGCGTTTCTTACCTTTGGTTTCCATGAGGTCGTGTCCCCGCTGGGTGACAAAGAGGCTGTCCGTGTCCCCGT
AGACCGACTTTATGGGCCTGTCCTCGAGCGGAGTGCCTCGGTCCTCTTCGTAGAGGAACCCAGCCCACTCTGATACA
AAAGCGCGTGTCCAGGCCAGCACAAAGGAGGCCACGTGGGAGGGGTAGCGGTCGTTGTCAACCAGGGGGTCCACCTT
CTCTACGGTATGTAAACACATGTCCCCCTCCTCCACATCCAAGAATGTGATTGGCTTGTAAGTGTAGGCCACGTGAC
CAGGGGTCCCCGCCGGGGGGGTATAAAAGGGGGCGGGCCTCTGTTCGTCCTCACTGTCTTCCGGATCGCTGTCCAGG
AGCGCCAGCTGTTGGGGTAGGTATTCCCTCTCGAAGGCGGGCATGACCTCTGCACTCAGGTTGTCAGTTTCTAGGAA
CGAGGAGGATTTGATATTGACAGTACCAGCAGAGATGCCTTTCATAAGACTCTCGTCCATCTGGTCAGAAAACACAA
TCTTCTTGTTGTCCAGCTTGGTAGCAAATGATCCATAGAGGGCATTGGATAGAAGCTTGGCGATGGAGCGCATGGTT
TGGTTCTTTTCCTTGTCCGCGCGCTCCTTGGCGGCGATGTTAAGCTGGACGTACTCGCGCGCCACACATTTCCATTC
AGGGAAGATGGTTGTCAGTTCATCCGGAACTATTCTGACTCGCCATCCCCTATTGTGCAGGGTTATCAGATCCACAC
TGGTGGCCACCTCGCCTCGGAGGGGCTCATTGGTCCAGCAGAGTCGACCTCCTTTTCTTGAACAGAAAGGGGGGAGG
GGGTCTAGCATGAGCTCATCAGGGGGGTCCGCATCTATGGTAAATATTCCCGGTAGCAAATCTTTGTCAAAATAGCT
GATGGTGGTGGGATCATCCAAGGTCATCTGCCATTCTCGAACTGCCAGCGCGCGCTCATAGGGGTTAAGAGGGGTGC
CCCAGGGCATGGGGTGGGTGAGCGCGGAGGCATACATGCCACAGATATCGTATACATAGAGGGGCTCTTCGAGGATG
CCGATGTAAGTGGGATAACAGCGCCCCCCTCTGATGCTTGCTCGCACATAGTCATAGAGTTCATGTGAGGGGGCGAG
AAGACCCGGGCCCAGATTGGTGCGGTTGGGTTTTTCCGCCCTGTAAACGATCTGGCGAAAGATGGCATGGGAATTTG
AAGAGATAGTAGGTCTCTGGAATATGTTAAAATGGGCATGAGGTAGGCCTACAGAGTCCCTTATGAAGTGGGCATAT
GACTCTTGCAGCTTGGCTACCAGCTCGGCGGTGACGAGTACATCCAGGGCACAGTAGTCGAGAGTTTCCTGGATGAT
GTCATAACGCGGTTGGCTTTTCTTTTCCCACAGCTCGCGGTTGAGAAGGTATTCTTCGCGATCCTTCCAGTACTCTT
CGAGGGGAAACCCGTCTTTTTCTGCACGGTAAGAGCCCAACATGTAGAACTGATTGACTGCCTTGTAGGGACAGCAT
CCCTTCTCCACTGGGAGAGAGTATGCTTGGGCTGCATTGCGCAGCGAGGTATGAGTGAGGGCAAAAGTGTCCCTGAC
CATGACTTTGAGGAATTGATACTTGAAGTCCATGTCATCACAGGCCCCCTGTTCCCAGAGTTGGAAGTCCACCCGCT
TCTTGTAGGCGGGGTTGGGCAAAGCGAAAGTAACATCATTGAAGAGGATCTTGCCGGCCCTGGGCATGAAATTTCGG
GTGATTCTGAAAGGCTGAGGGACCTCTGCTCGGTTATTGATAACCTGAGCGGCCAAGACGATCTCATCAAAGCCATT
GATGTTGTGCCCCACTATGTACAGTTCTAAGAATCGAGGGGTGCCCCTGACATGAGGCAGCTTCTTGAGTTCTTCAA
AAGTGAGATCTGTAGGGTCAGTGAGAGCATAGTGTTCGAGGGCCCATTCGTGCACGTGAGGGTTCGCTTTGAGGAAG
GAGGACCAGAGGTCCACTGCGAGTGCTGTTTGTAACTGGTCCCGGTATTGACGAAAATGCTGCCCGACTGCCATTTT
TTCTGGGGTGACGCAATAGAAGGTTTGGGGGTCCTGCCGCCAGCGATCCCACTTAAGTTTCATGGCGAGGTCATAGG
CGATGTTGACGAGCCGCTGGTCTCCAGAGAGTTTCATGACCAGCATGAAGGGGATTAGCTGCTTGCCAAAGGACCCC
ATCCAGGTGTAGGTTTCCACATCGTAGGTGAGGAAGAGCCTTTCTGTGCGAGGATGAGAGCCAATCGGGAAGAACTG
GATCTCCTGCCACCAGTTGGAGGAATGGCTGTTGATGTGATGGAAGTAGAACTCCCTGCGACGCGCCGAGCATTCAT
GCTTGTGCTTGTACAGACGGCCGCAGTACTCGCAGCGATTCACGGGATGCACCTCATGAATGAGTTGTACCTGACTT
CCTTTGACGAGAAATTTCAGTGGAAAATTGAGGCCTGGCGTTTGTACCTGGCGCTCTACTATGTTGTCTGCATCGGC
ATGACCATCTTCTGTCTCGATGGTGGTCATGCTGACGAGCCCTCGCGGGAGGCAAGTCCAGACCTCGGCGCGGCAGG
GGCGGAGCTCGAGGACGAGAGCGCGCAGGCCGGAGCTGTCCAGGGTCCTGAGACGCTGCGGAGTCAGGTTAGTAGGC
AGTGTCAGGAGATTGACTTGCATGATCTTTTCGAGGGCGTGAGGGAGGTTCAGATGGTACTTGATCTCCACGGGTCC
GTTGGTGGAGATGTCAATGGCTTGCAGGGTTCCGTGCCCCTTGGGCGCTACCACCGTGCCCTTGTTTTTCCTTTTGG
GCGGCGGTGGCTCTGTTGCTTCTTGCATGTTTAGGAGCGGTGTCGAGGGCGCGCACCGGGCGGCAGGGGCGGCTCGG
GACCCGGCGGCATGGCTGGCAGTGGTACGTCGGCGCCGCGCGCGGGTAGGTTCTGGTACTGCGCCCTGAGAAGACTC
GCATGTGCGACGACGCGGCGGTTGACATCCTGGATCTGACGCCTCTGGGTGAAAGCTACCGGCCCCGTGAGCTTGAA
CCTGAAAGAGAGTTCAACAGAATCAATCTCGGTATCGTTGACGGCGGCTTGCCTAAGGATTTCTTGCACGTCGCCAG
AGTTATCCTGGTAGGCGATCTCGGCCATGAACTGCTGGATCTCTTCCTCTTGAAGATCTCCGCGGCCCGCTCTCTCG
ACGGTGGCCGCTAGGTCGTTGGAGATGCGCCCAATGAGTTGAGAGAATGCATTCATGCCCGCCTCGTTCCAGACGCG
GCTGTAGACCACAGCCCCCACGGGATCTCTCGCGCGCATAACCACCTGGGCGAGGTTAAGCTCTACGTGGCGGGTGA
AGACCGCATAGTTGCATAGGCGCTGGAAAAGGTAGTTGAGTGTGGTGGCGATGTGCTCGGTGACGAAGAAATACATG
ATCCATCGTCTCAGCGGCATCTCGCTGACATCGCCCAGCGCTTCCAAGCGCTCCATGGCCTCGTAGAAGTCCACGGC
AAAGTTGAAAAACTGGGAGTTACGCGCGGACACGGTCAACTCCTCTTCCAGAAGACGGATGAGTTCGGCAATGGTGG
TGCGCACCTCGCGCTCGAAATCCCCCGGGATTTCTTCCTCAATCTCTTCTTCTTCCACTAACATCTCTTCCTCTTCA
GGTGGGGCTGCAGGAGGAGGGGGAACGCGGCGACGCCGGCGGCGCACGGGCAGACGGTCGATGAATCTTTCAATGAC
CTCTCCGCGGCGGCGGCGCATGGTCTCGGTGACGGCACGACCGTTCTCCCTGGGTCTCAGAGTGAAGACGCCTCCGC
GCATCTCCCTGAAGTGGTGACTGGGAGGCTCTCCGTTGGGCAGGGACACCGCGCTGATTATGCATTTTATCAATTGC
CCCGTAGGTACTCCGCGCAAGGACCTGATCGTCTCAAGATCCACGGGATCTGAAAACCTTTCGACGAAAGCGTCTAA
CCAGTCGCAATCGCAAGGTAGGCTGAGCACTGTTTCTTGCGGGCGGGGGCGGCTAGACGCTCGGTCGGGGTTCTCTC
TTTCTTTTCCTTCCTCCTCTTGGGAGGATGAGACGATGCTGCTGGTGATGAAATTAAAATAGGCAGTTTTGAGACGG
CGGATGGTGGCGAGGAGCACCAGGTCTTTGGGTCCGGCTTGTTGGATGCGCAGGCGATGGGCCATTCCCCAAGCATT
ATCCTGACATCTGGCCAGATCTTTATAGTAGTCTTGCATGAGTCGTTCCACGGGCACTTCTTCTTCGCCCGCTCTGC
CATGCATGCGAGTGATCCCGAACCCGCGCATGGGCTGGACAAGTGCCAGGTCCGCTACAACCCTTTCGGCGAGGATG
GCTTGCTGCACCTGGGTGAGGGTGGCTTGGAAGTCGTCAAAGTCCACAAAGCGGTGGTAGGCCCCGGTGTTGATTGT
GTAGGAGCAGTTGGCCATGACTGACCAGTTGACTGTCTGGTGCCCAGGGCGCACGAGCTCGGTGTACTTGAGGCGCG
AGTATGCGCGGGTGTCAAAGATGTAATCGTTGCAGGTGCGCACCAGGTACTGGTAGCCGATGAGAAAGTGTGGCGGT
GGCTGGCGGTACAGGGGCCATCGCTCTGTAGCCGGGGCTCCGGGGGCAAGGTCTTCCAGCATGAGGCGGTGGTAACC
GTAGATGTACCTGGACATCCAGGTGATACCGGAGGCGGTGGTGGATGCCCGCGGGAACTCGCGTACGCGGTTCCAGA
TGTTGCGCAGCGGCATGAAGTAGTTCATGGTAGGCACGGTTTGGCCCGTGAGACGTGCACAGTCGTTGATGCTCTAG
ACATACGGGCAAAAACGAAAGCGGTCAGCGGCTCGTCTCCGTGGCCTGGAGGCTAAGCGAACGGGTTGGGCTGCGCG
TGTACCCCGGTTCGAATCTCGGATCAGGCTGGAGCCGCAGCTAACGTGGTACTGGCACTCCCGTCTCGACCCAGGCC
TGCACAAAACCTCCAGGATACGGAGGCGGGTCGTTTTTTTGCTTTTTCCTGGATGGGAGCCAGTGCTGCGTCAAGCT
TTAGAACACTCAGTTCTCGGGGCTGGGAGTGGCTCGCGCCCGTAGTCTGGAGAATTAATCGCCAGGGTTGCGTTGCG
GTGTGCCCCGGTTCGAGTCTTAGCGCGCCGGATCGGCCGGTTTCCGCGACGTTTCTAAGACCCCGCCAGCCGACTTC
TCCAGTTTACGGGAGCGAGCCCTCTTTTTTTTTTTTGTTTTTTGTTGCCCAGATGCATCCCGTGCTGCGACAGATGC
GCCCCCAGCAACAGCCCCCTTCTCAGCAGCAGCTACAACAACAGCCACAAAAGGCTCTTCCTGCTCCTGTAACTACT
GCGGCTGCAGCCGTCAGCGGCGCGGGACAGCCCGCCTATGATCTGGAATTGGAAGAGGGCGAGGGACTGGCGCGCCT
GGGCGCACCATCGCCCGAGCGGCACCCGCCCAGCCGACTTCTCCAGTTTACGGGAGCGAGCCCTCTTTTTTTTTTTT
GTTTTTTGTTGCCCAGATGCATCCCGTGCTGCGACAGATGCGCCCCCAGCAACAGCCCCCTTCTCAGCAGCAGCTAC
AACAACAGCCACAAAAGGCTCTTCCTGCTCCTGTAACTACTGCGGCTGCAGCCGTCAGCGGCGCGGGACAGCCCGCC
TATGATCTGGAATTGGAAGAGGGCGAGGGACTGGCGCGCCTGGGCGCACCATCGCCCGAGCGGCACCCGCGGGTGCA
ACTGAAAAAGGACTCTCGCGAGGCGTACGTGCCCCAGCAGAACCTGTTCAGGGACAGGAGCGGTGAGGAGCCAGAGG
AGATGCGAGCATCTCGATTTAACGCGGGTCGCGAGCTGCGCCACGGTCTGGATCGAAGACGGGTGCTGCAAGACGAG
GATTTTGAGGTCGATGAAGTGACAGGGATCAGCCCAGCTAGGGCACATGTGGCCGCGGCCAACCTAGTCTCAGCCTA
CGAGCAGACCGTGAAGGAGGAGCGCAACTTCCAAAAATCTTTTAACAACCATGTGCGCACCCTGATCGCCCGCGAGG
AAGTGACCCTGGGTCTGATGCATCTGTGGGACCTGATGGAGGCTATCACCCAGAACCCCACTAGCAAACCACTGACA
GCTCAGCTGTTTCTGGTGGTTCAACATAGCAGGGACAACGAGGCATTCAGGGAGGCGTTGTTGAACATCACCGAGCC
TGATGGGAGATGGCTGTATGATCTGATCAACATCCTGCAAAGTATTATAGTGCAGGAACGTAGCCTGGGTTTGGCTG
AGAAAGTGGCAGCTATCAACTACTCGGTCTTGAGCCTGGGCAAATACTACGCTCGCAAGATCTACAAGACCCCCTAC
GTACCCATAGATAAGGAGGTAAAGATAGATGGGTTTTACATGCGCATGACTCTGAAGGTGCTGACTCTGAGCGACGA
TCTGGGGGTGTATCGCAATGACAGGATGCACCGCGCGGTGAGCGCCAGCAGGAGGCGCGAGCTGAGCGACAGAGAAC
TTATGCACAGCTTGCAAAGAGCTCTAACGGGGGCCGGGACTGATGGGGAGAACTACTTTGACATGGGAGCGGACTTG
CAATGGCAACCCAGTCGCAGGGCCATGGAGGCTGCAGGGTGTGAGCTTCCTTACATAGAAGAGGTGGATGAAGTCGA
GGACGAGGAGGGCGAGTACTTGGAAGACTGATGGCGCGACCCGTATTTTTGCTAGATGGAACAGCAGCAGGCACCGG
ACCCCGCAATGCGGGCGGCGCTGCAGAGCCAGCCGTCCGGCATTAACTCTTCGGACGATTGGACCCAGGCCATGCAA
CGCATAATGGCGCTGACGACCCGCAACCCCGAAGCCTTTAGACAGCAACCCCAGGCCAACCGCCTTTCGGCCATACT
GGAGGCCGTAGTGCCCTCCCGCTCCAACCCCACCCACGAGAAGGTCCTGGCTATCGTGAACGCGCTGGTGGAGAACA
AGGCCATCCGTCCCGATGAGGCCGGGCTGGTATACAATGCTCTCTTGGAGCGCGTGGCCCGTTACAACAGCAGCAAC
GTGCAAACCAACCTGGACAGGATGGTGACCGATGTGCGCGAGGCCGTGTCTCAGCGCGAGCGGTTCCAGCGCGGCGC
CAACTTGGGGTCGTTGGTAGCGCTAAACGCTTTCCTCAGCACCCAGCCCGCCAACGTGCCCCGTGGTCAGCAAGACT
ATACAAACTTTTTGAGTGCATTGAGACTCATGGTAGCTGAGGTGCCCCAGAGCGAGGTGTACCAGTCCGGGCCAGAT
TACTTCTTCCAGACCAGCAGACAGGGCTTGCAGACAGTGAACCTGACTCAGGCTTTCAAGAACCTGAAGGGTCTGTG
GGGAGTGCACGCCCCAGTAGGAGATCGCGCGACCGTGTCTAGCTTGCTGACTCCCAACTCCCGCCTGCTGCTGCTGC
TGGTATCCCCCTTTACTGACAGCGGTAGCATCGACCGCAACTCGTACTTGGGCTACCTGCTTAACCTGTATCGCGAG
GCCATAGGGCAGAGCCAGGTGGACGAGCAGACCTATCAAGAAATCACCCAAGTGAGCCGCGCCCTGGGTCAGGAAGA
CACGGGCAGTTTGGAAGCCACCCTGAACTTCTTGCTAACCAACCGGTCGCAGAAGATCCCTCCTCAGTATGCGCTTA
CCGCTGAGGAGGAGCGGATCCTCAGATACGTGCAACAGAGCGTTGGACTGTTTCTGATGCAGGAGGGGGCGACACCT
ACCGCCGCGCTGGACATGACAGCTCGAAACATGGAGCCCAGCATGTATGCTAGTAACAGGCCTTTCATTAACAAACT
GCTGGACTACCTGCACAGGGCGGCCGCCATGAACTCTGATTATTTCACCAATGCTATCCTGAACCCACACTGGCTGC
CCCCACCTGGTTTCTACACTGGCGAGTACGACATGCCCGACCCCAATGACGGGTTCCTGTGGGACGATGTGGACAGC
AGCATATTCTCCCCGCCTCCCGGTTATACAGTTTGGAAGAAGGAAGGGGGCGATAGAAGACACTCTTCCGTGTCGCT
GTCCGGAACGGCTGGTGCTGCCGCGACCGTGCCCGAAGCTGCAAGTCCTTTCCCTAGCTTGCCCTTTTCACTAAACA
GCGTTCGCAGCAGTGAACTGGGGAGAATAACCCGCCCGCGCTTGATGGGCGAGGATGAGTACTTGAATGACTCTTTG
CTGAGGCCAGAGAGGGAAAAGAACTTCCCCAACAATGGAATAGAGAGTCTGGTGGATAAGATGAGTAGATGGAAGAC
CTATGCGCAGGATCACAGAGACGAGCCCAGGATATTGGGGGCTACAAGCAGACCGACCCGTAGACGCCAGCGCCACG
ACAGACAGATGGGTCTTGTGTGGGACGATGAGGACTCTGCCGATGATAGCAGCGTGTTGGACTTGGGTGGAAGAGGA
GGGGGCAACCCGTTCGCTCATCTGCGTCCCAGATTCGGGCGCATGTTGTAAAAGTGAAAGTAAAATAAAAATGCAAC
TCACCAAGGCCATGGCGACCGAGCGTGCGTTCGTTCTTTTTTGTTATCTGTGTCTAGTACGATGAGGAGACGAGCCG
TGCTAGGCGGAGCGGTGGTGTATCCGGAGGGTCCTCCTCCTTCTTACGAGAGCGTGATGCAGCAACAGGCGGCGATG
ATACAGCCCCCACTGGAGGTTCCCTTCGTACCCCCGCGGTACCTGGCGCCTACGGAAGGGAGAAACAGCATTCGTTA
CTCGGAGCTGTCGCCCCTGTACGATACCACCAAGTTGTATCTGGTTGACAACAAGTCGGCGGACATCGCCTCCCTGA
ACTATCAGAACGACCACAGCAACTTCCTGACCACGGTGGTGCAGAACAATGACTTTACCCCCACGGAGGCTAGCACC
CAGACCATCAACTTTGACGAACGGTCGCGATGGGGCGGTCATCTGAAGACCATCATGCACACCAACATGCCCAACGT
GAACGAGTACATGTTCAGCAACAAGTTCAAGGCGAGGGTGATGGTGTCCAGAAAAGCTCCTGAAGGTGTTACAGTAA
ATGACACCTATGATCATAAAGAGGATATCTTGAAGTATGAGTGGTTTGAGTTCATTTTACCAGAAGGCAACTTTTCA
GCCACCATGACGATCGACCTGATGAACAATGCCATCATTGACAACTACCTGGAAATTGGCAGACAGAATGGAGTGCT
GGAAAGTGACATTGGTGTTAAGTTTGACACTAGAAATTTCAGGCTCGGGTGGGACCCCGAAACTAAGTTGATTATGC
CAGGAGTCTACACTTATGAGGCATTCCATCCTGACATTGTATTGCTGCCTGGTTGCGGGGTAGACTTTACTGAAAGC
CGACTTAGCAACTTGCTTGGCATCAGGAAGAGACATCCATTCCAGGAGGGTTTCAAAATCATGTATGAAGATCTTGA
AGGGGGTAATATTCCTGCCCTTTTGGATGTCACTGCCTATGAGGAAAGCAAAAAGGATACCACTACTGAAACAACCA
CACTGGCTGTTGCAGAGGAAACTAGTGAAGATGATAATATAACTAGAGGAGATACCTATATAACAGAAAAACACAAA
CGTGAAGCTGCAGCTGCTGAAGTTAAAAAAGAGTTAAAGATCCAACCTCTAGAAAAAGACAGCAAGAGTAGAAGCTA
CAATGTCTTGGAAGACAAAATCAACACGGCCTACCGCAGTTGGTACCTGTCCTACAATTACGGTAACCCTAAGAAAG
GAATAAGGTCTTGGACACTGCTCACCACTTCAGATGTCACCTGTGGGGCAGAGCAGGTTTACTGGTCGCTCCCTGAC
ATGATGCAAGACCCAGTCACGTTCCGCTCCACAAGACAAGTCAACAACTACCCAGTGGTGGGTGCAGAGCTTATGCC
CGTCTTCTCAAAGAGTTTCTACAATGAGCAAGCCGTGTACTCTCAGCAGCTCCGACAGGCCACTTCGCTCACGCACG
TCTTCAACCGCTTCCCTGAGAACCAGATCCTCATCCGCCCGCCGGCGCCCACAATTACCACCGTCAGTGAAAACGTT
CCTGCTCTCACAGATCACGGGACCCTGCCGTTACGCAGCAGTATCCGGGGAGTCCAGCGCGTGACCGTTACTGACGC
CAGACGCCGCACCTGTCCCTACGTTTACAAGGCCCTGGGCATAGTCGCGCCGCGCGTTCTTTCAAGCCGCACTTTCT
AAAAAAAAAAAATGTCCATTCTCATCTCGCCCAGTAATAATACCGGTTGGGGACTGTATGCGCCCACCAAGATGTAT
GGAGGCGCCCGCAAACGCTCTACCCAGCACCCTGTGCGCGTTCGCGGTCATTTCCGCGCTCCCTGGGGCGCACTCAA
GGGTCGTACCCGCACTCGGACCACGGTCGATGATGTGATCGACCAGGTGGTCGCCGATGCTCGTAATTATACTCCTA
CTGCGCCTACATCTACTGTGGATGCAGTTATTGACAGTGTGGTGGCAGACGCCCGCGCCTATGCTCGCCGGAAGAGC
CGAAGGAGGCGCATCGCCAGGCGCCACAGGGCTACTCCCGCCATGCGAGCTGCAAAAGCTATTCTGCGGAGGGCCAA
ACGTGTGGGGCGAAGAGCCATGCTTAGAGCGGCCAGACGCGCGGCTTCAGGTGCCAGCAGCGGCAGGTCCCGCAGGC
GCGCGGCCACGGCGGCAGCAGCGGCCATTGCCAACATGGCCCAACCGCGAAGAGGCAATGTGTACTGGGTGCGTGAT
GCCACTACCGGCCAGCGCGTGCCCGTGCGCACTCGCCCCCCTCGCACTTAGAAGATACTGAGCAGTCTCCGATGTTG
TGTCCCAGCGGCAAGTATGTCCAAGCGCAAATACAAGGAAGAGATGCTCCAGGTCATCGCGCCTGAAATCTACGGTC
CACCGGTGAAGGATGAAAAAAAGCCCCGCAAAATCAAGCGGGTCAAAAATAACAAAAAGGAAGAAGATGACGATGAT
GGGCTGGTGGAGTTTGTGCGCGAGTTCGCCCCAAGACGGCGCGTGCAGTGGCGCGGGCGCAAAGTGCGTCAAGTGCT
CAGACCCGGGACCACTGTGGTTTTTACACCCGGCGAGCGTTCCAGCACTACTTTTAAACGGTCCTATGATGAGGTGT
ACGGGGATGACAATATTCTTGAGCAGGCGGCAGACCGCCTTGACGAGTTTGCTTATGGCAAGCGCACTAGATCCAGT
CCCAAAGAGGAGGCGGTGTCCATTCCTTTGGATCATGGAAATCCCACCCCCAGCCTCAAACCAGTCACCCTGCAGCA
AGTGCTGCCCGTGCCTGCGCGGAGAGGCGTAAAGCGCGAGGGTGAGGACCTGTATCCCACCATGCAGCTAATGGTGC
CCAAGCGCCAGAGGCTAGAAGACGTACTGGAGAAAATGAAAGTGGATGCCGATATCCAGCCTGAGGTCAAAGTAAGA
CCTATCAAGGAAGTGGCGCCAGGTTTGGGAGTACAAACCTTCGACATCAAGATTCCCACCGAGTCCATGGAAGTGCA
GACCGAACCTGCAAAACCCACAACCACCTCAATTGAGGTGCAAACGGAACCCTGGATGCCCGCGCCCGTTGCCGCCC
CCAGCACCACTCGAAGATCACGACGAAAGTACGGCCCAGCAAGTCTGCTAATGCCCAACTATGCTCTGCACCCATCC
ATCATTCCCACTCCGGGTTACAGAGGCACTCGCTACTATCGAAACCGGAGCAACACCTCTCGCCGCCGCAAACCACC
TGCAAGTCGCACTCGCCGTCGCCGCCGCCGCAACACTGCCAGCAAATTGACTCCCGCCGCCCTGGTGCGGAGAGTGT
ACCGCGATGGTCGCGCTGAACCTCTGACGCTGCCGCGCGCGCGCTACCATCCAAGCATCACCACTTAATGACTGTTG
ACGCTGCCTCCTTGCAGATATGGCCCTCACTTGCCGCCTTCGCGTCCCCATTACTGGCTACCGAGGAAGAAACTCGC
GCCGTAGAAGGATGTTGGGGCGAGGGATGCGCCGCCACAGACGAAGGCGCGCTATCAGCAGACGATTAGGGGGTGGC
TTTTTGCCAGCTCTTATACCCATCATCGCCGCAGCGATCGGGGCGATACCAGGCATAGCTTCCGTGGCGGTTCAGGC
CTCGCAGCGCCACTAACATTGGAAAAACTTATAAATAAAAAATAGAATGGACTCTGACGCTCCTGGTCCTGTGACTA
TGTTTTTGTAGAGATGGAAGACATCAATTTTTCATCCCTGGCTCCGCGACACGGCACGAGGCCGTACATGGGCACCT
GGAGCGACATCGGCACGAGCCAACTGAACGGGGGCGCCTTCAATTGGAGCAGTATCTGGAGCGGGCTTAAAAATTTT
GGCTCGACCGTAAAAACCTATGGGAACAAAGCTTGGAACAGCAGCACAGGGCAGGCTCTGAGAAATAAGCTTAAGGA
ACAAAACTTCCAACAGAAGGTGGTCGATGGGATCGCCTCTGGTATTAACGGCGTAGTGGATCTGGCCAACCAGGCTG
TACAAAAACAGATAAACAGCCGCCTGGACCCGCCGCCCGCAACCCCTGGTGAAATGGAAGTGGAGGAAGAACTTCCT
CCGCTGGAAAAGCGGGGCGACAAGCGTCCGCGACCCGAGCTAGAGCACACGCTGGTGACGCGCGCAGACGAGCCCCC
TTCATACGAGGAGGCAGTAAAGCTCGGAATGCCCACTACCAGGCCCGTAGCTCACATGGCTACCGGGGTGATGAAAC
CTTCTGAGTTACATCGACCCGCCACCTTGGACTTGCCTCCTCCCCCTGCTTCTGCGGCGCCTGTTCCCAAACCTGTC
GCTACCAGAAAGCCCACCGCCGTACAGCCCGTTGCCGTAGCCAGACCGCGTCCTGGGGGCACACCGCGCCCGAAAGC
AAACTGGCAAAGTACTCTGAACAGCATCGTGGGTCTGGGCGTGCAGAGTGTAAAGCGCCGTCGCTGCTATTAATTAA
ATATGGAGTAGCGCTTAACTTGCTTGTCTGTGTGTATGTATCATCACCACGCCGCCGCAGCAGAGGAGAAAGGAAGA
GGTCGCGCGCCGAGGCTGAGTTGCTTTCAAGATGGCCACCCCATCGATGATGCCCCAATGGGCATACATGCACATCG
CCGGACAGGATGCTTCGGAGTACCTGAGTCCGGGTCTGGTGCAGTTCGCCCGTGCAACAGACACCTACTTCAGTATG
GGGAACAAGTTTAGAAACCCCACAGTGGCGCCCACCCACGATGTGACCACCGACCGTAGCCAGCGACTGATGCTGCG
CTTCGTGCCCGTTGACCGGGAGGACAATACATACTCTTACAAAGTGCGGTACACCCTCGCCGTGGGCGACAACAGAG
TGCTTGACATGGCCAGCACATTCTTTGACATTAGGGGGGTGCTTGATAGAGGTCCTAGCTTCAAGCCATATTCCGGC
ACAGCTTACAATTCACTGGCTCCTAAGGGCGCGCCTAACACATCTCAGTGGATAGTTACAACGGGAGAAGACAATGC
CACCACATACACATTTGGCATTGCTTCCACGAAGGGAGACAATATTACTAAGGAAGGTTTAGAAATTGGGAAAGACA
TTACTGCAGACAACAAGCCCATTTATGCCGATAAAACATATCAGCCAGAGCCTCAAGTTGGAGAAGAATCATGGACT
GATATTGATGGAACAAATGAAAAATTTGGAGGTAGAGCTCTTAAACCAGCTACTAAAATGAAGCCATGCTACGGGTC
TTTTGCAAGACCTACAAACATAAAAGGGGGCCAAGCTAAAAACAGAAAAGTAACACCAACCGAAGGAGATGTTGAAG
CTGAGGAGCCAGATATTGATATGGAATTTTTCGATGGTAGAGAAGCTGCTGACGCTTTTTCGCCTGAAATTGTGCTT
TACACGGAAAATGTCAATTTGGAAACTCCAGACAGCCATGTGGTATACAAGCCAGGAACTTCTGATGGTAACTCTCA
TGCAAATTTGGGTCAACAAGCCATGCCTAACAGACCCAATTACATTGGCTTCAGGGATAACTTTGTAGGTCTTATGT
ACTACAACAGTACTGGAAATATGGGAGTTTTGGCCGGCCAAGCATCACAACTGAATGCAGTGGTTGACTTGCAGGAC
AGAAACACTGAACTGTCATATCAGCTTTTGCTTGATTCTCTGGGAGACAGAAGCAGATACTTCAGCATGTGGAATCA
GGCTGTGGACAGCTATGATCCCGATGTTCGTATTATTGAAAATCATGGCGTCGAGGATGAACTGCCTAATTACTGTT
TTCCTCTGGATGGCATAGGACCAGGGAACAAATATCAAGGCATTAAACCTAGAGACACTGCATGGGAAAAAGATACT
AAAGTTTCTACAGCTAATGAAATAGCCATAGGCAACAATCTGGCTATGGAAATTAATATCCAAGCTAATCTTTGGAG
AAGTTTTCTGTACTCCAATGTGGCTTTGTACCTTCCAGATGTTTACAAGTACACGCCAACTAACATTACTCTGCCCG
CTAACACCAACACCTATGAGTACATGAACGGGCGAGTGGTTTCCCCATCTCTGGTCGATTCATACATCAACATTGGC
GCCAGGTGGTCTCTTGACCCAATGGACAATGTGAATCCATTTAACCACCACCGCAATGCTGGCCTACGCTACCGGTC
CATGCTTCTGGGCAATGGCCGTTATGTGCCTTTCCACATACAAGTGCCTCAAAAATTCTTTGCTGTCAAGAACCTAC
TTCTTCTACCTGGCTCCTACACCTATGAGTGGAACTTCAGAAAGGATGTGAACATGGTCCTGCAAAGTTCCCTTGGA
AATGACCTCAGAACAGATGGTGCTAACATAAGTTTCACCAGCATCAACCTCTATGCCACCTTCTTCCCCATGGCTCA
CAACACCGCTTCAACTCTTGAAGCCATGCTGCGCAACGATACCAATGATCAGTCATTCAACGACTACCTCTCTGCAG
CTAACATGCTTTACCCCATCCCTGCCAATGCAACCAACATTCCAATTTCCATCCCATCTCGCAACTGGGCAGCCTTC
AGGGGCTGGTCCTTCACCAGACTCAAAACCAAGGAGACTCCATCTCTTGGATCAGGGTTCGATCCCTACTTCGTTTA
TTCTGGATCTATTCCCTACCTGGATGGCACTTTTTACCTTAACCACACTTTCAAGAAGGTCTCCATCATGTTTGACT
CCTCAGTCAGCTGGCCTGGCAATGACAGGCTGTTGTCTCCAAATGAGTTTGAAATCAAGCGCACTGTGGATGGGGAA
GGATACAATGTGGCCCAATGCAACATGACCAAAGACTGGTTCCTGGTTCAGATGCTTGCCAACTACAACATTGGCTA
CCAGGGCTTTTACATCCCTGAGGGATACAAGGATCGCATGTACTCCTTTTTCAGAAACTTCCAGCCTATGAGCAGGC
AGGTGGTTGATGAGGTTAATTACACTGACTACAAAGCCGTCACCTTACCATATCAACACAACAACTCTGGCTTTGTA
GGATACCTTGCGCCTACTATGAGACAAGGGGAACCTTACCCAGCCAATTATCCATACCCGCTCATCGGAACTACTGC
CGTTAAAAGTGTTACCCAAAAAAAGTTCCTGTGCGACAGGACCATGTGGCGCATACCGTTCTCCAGCAACTTCATGT
CCATGGGAGCCCTTACGGACCTGGGACAGAACCTGCTCTATGCCAACTCGGCCCATGCGCTGGACATGACTTTTGAG
GTGGATCCCATGGATGAGCCCACCCTGCTTTATCTTCTTTTCGAAGTCTTCGACGTGGTCAGAGTGCACCAGCCACA
CCGCGGCGTCATCGAGGCCGTCTACCTGCGCACACCGTTCTCGGCCGGCAACGCCACCACATAAGAAGCCTCTTGCT
TCTTGCAAGCAGCAGCTGCAGCCATGTCATGCGGGTCCGGAAACGGCTCCAGCGAGCAAGAGCTCAAAGCCATCGTC
CGAGACCTGGGTTGCGGACCCTATTTCCTGGGAACCTTTGACAAGCGTTTCCCGGGGTTCATGGCCCCCGACAAGCT
CGCCTGCGCCATAGTCAACACTGCCGGACGCGAGACGGGGGGAGAGCACTGGCTGGCTTTTGGTTGGAACCCGCGCT
CCAACACCTGCTACCTTTTTGATCCTTTTGGGTTCTCGGATGAGCGACTCAAACAGATTTACCAGTTTGAGTACGAG
GGGCTCCTGCGCCGCAGTGCCCTTGCTACCAAAGACCGCTGCATCACCCTGGAAAAGTCCACCCAGAGCGTGCAGGG
CCCACGCTCAGCCGCCTGTGGACTTTTTTGCTGTATGTTCCTTCATGCCTTTGTGCACTGGCCCGACCGCCCCATGA
ACGGAAACCCCACCATGAAGTTGCTGACTGGGGTGCCCAACAGCATGCTCCAATCTCCCCAAGTGCAGCCCACCCTG
CGCCGCAACCAGGAGGCGCTATATCGCTTCCTAAACACCCACTCATCTTACTTTCGTTCTCACCGCGCACGCATCGA
AAGGGCCACCGCGTTTGACCGTATGGATATGCAATAAGTCATGTAAAACCGTGTTCAATAAAAAGCACTTTATTTTT
ACATGCACTAAGGCTCTCGTTTTTTACTCATTCGTTTTCATTATTCACTCAGAAATCAAATGGGTTCTGGCGGGAGT
CAAAGTGACCCGCGGGCAGGGATACGTTGCGGAACTGTAACCTGTTCTGCCACTTGAACTCGGGGATCACCAACTTG
GGAACTGGAATCTCGGGAAAGGTGTCTTGCCACAACTTTCTGGTCAGCTGCAGGGCGCCAAGTAGGTCAGGAGCAGA
GATCTTGAAATCACAGTTGGGACCGGCATTCTGGACACGGGAGTTGCGGTACACTGGGTTGCAACACTGGAACACCA
TCAAGGCTGGGTGTCTCACGCTTGCCAGCACGGTCGGGTCACTGATGGTAGTCACATCCAAGTCTTCAGCATTGGCC
ATCCCAAAGGGGGTCATCTTACAGGTCTGCCTGCCCATCACGGGAGCGCAGCCTGGCTTGTGGTTGCAATCGCAATG
AATGGGGATCAGCATCATCCTGGCTTGGTCGGGGGTTATCCCTGGGTACACGGCCTTCATGAAGGCTTCGTACTGCT
TGAAAGCTTCCTGAGCCTTACTTCCCTCGGTATAGAACATCCCACAGGACTTGCTGGAAAATTGATTAGTAGCACAG
TTGGCATCATTTACACAGCAGCGGGCATCGTTGTTGGCCAACTGGACCACATTTCTGCCCCAGCGGTTCTGGGTGAT
CTTGGCTCTGTCTGGGTTCTCCTTCATAGCGCGCTGTCCGTTCTCGCTCGCCACATCCATCTCGATAATGTGGTCCT
TCTGAATCATGATAGTGCCATGCAGGCATTTCACCTTGCCTTCATAATCGGTGCATCCATGAGCCCACAGAGCGCAC
CCGGTGCACTCCCAACTATTGTGGGCGATCTCAGAATAAGAATGTACCAATCCCTGCATGAATCTTCCCATCATCGC
TGTCAGGGTCTTCATGCTACTAAATGTCAGCGGGATGCCACGGTGCTCCTCGTTCACATACTGGTGGCAGATACGCT
TGTACTGCTCGTGCTGCTCTGGCATCAGCTTGAAAGAGGTTCTCAGGTCATTATCCAGCCTGTACCTCTCCATTAGC
ACAGCCATCACTTCCATGCCCTTCTCCCAGGCAGATACCAGGGGCAAGCTCAAAGGATTCCTAACAGCAATAGAAGT
AGCTCCTTTAGCTATAGGGTCATTCTTGTCGATCTTCTCAACACTTCTCTTGCCATCCTTCTCAATGATGCGCACCG
GGGGGTAGCTGAAGCCCACGGCCACCAACTGAGCCTGTTCTCTTTCTTCTTCGCTGTCGTGGCCGATGTCTTGCAGA
GGGACATGCTTGGTCTTTCTGGGCTTCTTCTTGGGAGGGATCGGGGGAGGACTGTTGCTCCGTTCCGGAGACAGGGA
TGACCGCGAAGTTTCGCTTACCAGTACCACCTGGCTCTCGATAGAAGAATCGGACCCCACGCGACGGTAGGTGTTCC
TCTTCGGGGGCAGAGGTGGAGGCGACTGAGATGGGCTGCGGTCTGGCCTTGGAAGCGGATGGCTGGCAGAGCCCATT
CCGCGTTCGGGGGTGTGCTCCCGTTGGCGGTCGCTTGACTGATTTCCTCCGCGGCTGGCCATTGTGTTCTCCTAGGC
AGAGAAACAACAGACATGGAAACTCAGCCATCACTGCCAACATCGCTGCAAGCGCCATCACACCTCGCCCCCAGCAG
CGACGAGGAGGAGAGCTTAACCACCCCACCACCCAGTCCAGCTACCACCACCTCTACCCTCGATGATGAGGAGGAGG
AGGTCGACGCAGCCCAGGAGATGCAGGCGCAGGATAATGTGAAAGCGGAAGAGATTGAGGCAGATGTCGAGCAGGAC
CCGGGCTATGTGACACCGGCGGAGCACGAGGAGGAGCTGAAACGTTTTATAGACAGAGAGGATGACGACCGCCCAGA
GCATCAAGCAGATGGCGATCACCAGGAGGCTGGCATCGGGGATCAAGTTGCCGACTACCTCACCGGGCTTGGGGGGG
AAGACGTGCTCCTCAAACATCTAGCAAGGCAGTCGAACATAGTTAAAGACGCACTACTCGACCTCACCGAAGTGCCC
ATCAGTGTGGAAGAGCTTAGCCGCGCCTACGAGCTGAACCTCTTTTCGCCTCACATACCCCCCAAGCGGCAGCCAAA
CGGCACCTGCGAGGCCAACCCTCGACTGAACTTCTATCCAGCTTTTACTGTCCCCGAAGTGCTGGCCACCTACCACA
TCTTTTTTAAGAACCAAAAGATTCCAGTCTCCTGCCGCGCCAACCGCACCCGCGCCGATGCCCTTCTCAACTTGGGT
CCGGGAGCTCGCTTACCTGATATAGCTTCCTTGGAAGAGGTTCCAAAAATCTTTGAGGGTCTGGGAAGTGATGAGAC
TCGGGCCGCAAATGCTCTGCAACAGGGAGAGAATGGCATGGATGAACATCACAGCGCTTTAGTGGAACTGGAGGGTG
ACAATGCCCGGCTTGCAGTGCTCAAGCGCAGTATCGTGGTCACCCATTTTGCCTACCCCGCTGTTAACCTGCCCCCC
AAAGTTATGAGCGCTGTTATGGACCATCTGCTCATCAAACGAGCAGGTCCACTTTCAGAAAACCAGAACATGCAGGA
TCCAGACGCCTCGGACGAGGGCAAGCCGGTAGTCAGTGACGAGCAGCTATCTCGCTGGCTGGGTACCAACTCCCCCC
GAGATTTGGAAGAGAGGCGCAAGCTTATGATGGCTGTAGTGCTAGTAACTGTGGAGCTGGAGTGTTTGCGCCGCTTT
TTCACCGACCCCGAGACCCTGCGCAAGCTAGAGGAGAACCTGCACTACACCTTTAGACATGGCTTCGTGCGGCAGGC
ATGCAAGATCTCCAACGTGGAGCTTACCAACCTGGTTTCTTACATGGGCATTTTGCATGAGAACCGGCTAGGGCAGA
GCGTCCTGCACACCACCCTTAAAGGGGAGGCCCGCCGTGACTACATCCGAGACTGTGTCTACCTCTACCTCTGCCAT
ACCTGGCAGACTGGCATGGGTGTGTGGCAACAGTGTTTGGAAGAGCAGAACCTAAAAGAGCTGGACAAGCTCTTGCA
GAGATCCCTCAAAGCCCTGTGGACAGGTTTTGATGAGCGCACCGTCGCCTCGGACCTGGCAGACATCATCTTCCCCG
AGCGTCTCAGGGTTACTCTGCGAAACGGCCTGCCAGACTTTATGAGCCAGAGCATGCTTAACAACTTTCGCTCTTTC
ATCCTGGAACGCTCCGGTATCCTGCCTGCCACCTGCTGTGCGCTGCCCTCCGACTTTGTGCCTCTCACCTACCGCGA
GTGCCCACCGCCGCTATGGAGCCACTGCTACCTGTTCCGCCTGGCCAACTACCTCTCCTACCACTCGGATGTTATAG
AGGATGTGAGCGGAGACGGTCTGCTGGAATGCCACTGCCGCTGCAATCTTTGCACACCCCACCGCTCCCTTGCCTGC
AACCCCCAGTTGCTGAGCGAGACCCAGATCATCGGCACCTTCGAGTTGCAGGGTCCCAGCAGTGAAGGCGAGGGGTC
TTCTCCGGGGCAGAGTCTGAAACTGACACCGGGGCTGTGGACCTCCGCCTACCTGCGCAAGTTTCATCCCGAGGATT
ACCACCCCTATGAGATCAGGTTCTATGAGGACCAGTCACATCCTCCCAAAGTCGAGCTCTCAGCCTGCGTCATCACC
CAGGGAGCAATTCTGGCCCAATTGCAAGCCATCCAAAAATCCCGCCAAGAATTTCTACTGAAAAAGGGAAGCGGGGT
CTACCTTGACCCCCAGACCGGTGAGGAGCTCAACACAAGGTTCCCCCAGGATGTCCCATCGCCGAGGAAGCAAGAAG
CTGAAGGTGCAGCTGACGCCCCCAGAGGATATGGAGGAAGACTGGGACAGTCAGGCAGAGGAGGAGATGGAAGATTG
GGACAGCCAGGCAGAGGAGGTGGACAGCCTGGAGGAAGACAGTTTGGAGGAGGAAGACGAGGAGGCAGAGGAGGTGG
AAGAAGCAACCGCCGCCAAACAGTTGTCATCGGCGGCGGAGACAAGCAAGTCCCCAGACAGCAGCACGGCTACCATC
TCCGCTCCGGGTCGGGGGGTCCAGCGGCGGCCCAACAGTAGATGGGACGAGACCGGGCGATTCCCAAACCCGACCAC
CGCTTCCAAGACCGGTAAGAAGGAGCGACAGGGATACAAGTCCTGGCGTGGACATAAAAACGCTATCATCTCCTGCT
TGCATGAATGCGGGGGCAACATATCCTTCACCCGGCGATACCTGCTCTTCCACCACGGTGTGAACTTCCCCCGCAAT
ATCTTGCATTACTACCGTCACCTCCACAGCCCCTACTGCAGTCAGCAAGTCCCGGCAACCCCGACAGAAAAAGACAG
CAGCGACAACGGTGACCAGAAAACCAGCAGTTAGAAAATCTACAACAAGTGCAGCAGGAGGAGGACTGAGGATCACA
GCGAACGAGCCAGCGCAGACCAGAGAGCTGAGGAACCGGATCTTTCCAACCCTCTATGCCATCTTCCAGCAGAGTCG
GGGGCAAGAGCAGGAACTGAAAGTAAAAAACCGATCTCTGCGCTCGCTCACCAGAAGTTGTTTGTATCACAAGAGCG
AAGACCAACTTCAGCGCACTCTCGAGGACGCCGAGGCTCTCTTCAACAAGTACTGCGCGCTGACTCTTAAAGAGTAG
CCCTTGCCCGCGCTTATTCGAAAACGGCGGGAATCACGTCACCCTTGGCACCTGTCCTTTGCCCTAGTCATGAGTAA
AGAGATTCCCACGCCTTACATGTGGAGCTATCAGCCCCAAATGGGGTTGGCAGCAGGCGCCTCCCAGGACTACTCCA
CCCGCATGAATTGGCTTAGCGCCGGGCCCTCAATGATATCACGGGTTAATGATATACGAGCTTATCGAAACCAGTTA
CTCCTAGAACAGTCAGCTCTCACCACCACACCCCGCCAACACCTTAATCCCCGAAATTGGCCCGCCGCCCTGGTGTA
CCAGGAAACTCCCGCTCCCACCACCGTACTACTTCCTCGAGACGCCCAGGCCGAAGTTCAGATGACTAACGCAGGTG
TACAGCTGGCGGGCGGTTCCGCCCTATGTCGTCACCGACCTCAACAGAGTATAAAACGCCTGGTGATCAGAGGCCGA
GGTATCCAGCTCAACGACGAGTCGGTTAGCTCTTCGCTTGGTCTGCGACCAGACGGAGTCTTCCAGATCGCCGGCTG
TGGGAGATCTTCCTTCACCCCTCGTCAGGCTGTACTGACTTTGGAGAGTTCGTCCTCGCAGCCCCGCTCGGGCGGCA
TCGGAACTCTCCAGTTCGTGGAGGAGTTTACTCCCTCTGTCTACTTCAACCCCTTCTCCGGCTCTCCTGGCCAGTAC
CCAGACGAGTTCATACCGAACTTCGACGCAATCAGCGAGTCAGTGGATGGCTATGATTGATGTCTAATGGTGGCGCG
GCTGAGCTAGCTCGACTGCGACACCTAGACCACTGCCGCCGCTTTCGCTGCTTCGCCCGGGAACTCACCGAGTTCAT
CTACTTCGAACTCCCCGAGGAGCACCCTCAGGGTCCGGCCCACGGAGTGCGGATTACCATCGAAGGGGGAATAGACT
CTCGCCTGCATCGAATCTTCTCCCAGCGACCCGTGCTGATTGAGCGCGACCAGGGAAATACAACCATCTCCATTTAC
TGCATCTGTAACCACCCCGGATTGCATGAAAGCCTTTGCTGTCTTGTTTGTGCTGAGTTTAATAAAAACTGAGTTAA
GACCCTCCTACGGACTACCGCTTCTTCAATCAGGACTTTACAACACCAACCAGATCTTCCAGAAGACCCAGACCCTT
CCTCCTCTGATCCAGGACTCTAACTCTACCTTACCAGCACCATCCACTACTAACCTTCCCGAAACTAACAAGCTTGG
ATCTCATCTGCAACACCGCCTTTCACGAAGCCTTCTTTCTGCCAATACTACCACTCCCAAAACCGGAGGTGAGCTCC
GCGGTCTCCCTACTGACGACCCCTGGGTGGTAGCGGGTTTTGTAACGTTAGGAGTAGTTGCGGGTGGGCTTGTGCTA
ATCCTTTGCTACCTATACATACCTTGCTGTGCATATTTAGTCATATTGCGCTGTTGGTTTAAAAAATGGGGGCCATA
TTAGTCGTGCTTGCTTTACTTTCGCTTTTGGGTCTGGGCTCTGCTAATCTCAATCCTCTTGATCACGATCCATGTCT
AGACTTCGACCCAGAAAACTGCACACTTACTTTTGCACCCGACACAAGCCGTCTCTGTGGAGTTCTTATTAAGTGCG
GATGGGACTGCAGGTCCGTTGAAATTACACATAATAATAAAACATGGAACAATACCTTATCCACCACATGGGAGCCA
GGAGTTCCCGAGTGGTATACTGTCTCTGTCCGAGGTCCTGACGGTTCCATCCGCATTAGTAACAACACTTTTATTTT
TTCTGAAATGTGCGATCTGGCCATGTTCATGAGCAGACAGTATGACCTATGGCCTCCCAGCAAAGAGAACATTGTGG
CATTTTCCATTGCTTATTGCTTGGTAACATGCATCATCACTGCTATCATTTGTGTGTGCATACACTTGCTTATAGTT
ATTCGCCCTAGACAAAGCAATAAGGAAAAAGAGAAAATGCCTTAACCTTTTTACTCATACCTTTTCTTTACAGCATG
GCTTTTGTTACAGCTCTAATTATTGCCAACATTGTCACTGTCGCTCACGGGCAAACAATTATCCATATTACCTTAGG
ACATAATCACACCCTTGTAGGGCCCCCAATTACTTCAGAGGTTATTTGGACCAAACTTGGAAGTGTTGATTATTTTG
ATATAATTTGCAACAAAACTAAACCAATATTTGTAATCTGTAACAGACAAAATCTCACGTTAATTAATGTTAGCAAA
ATTTATAACGGTTACTATTATGGTTATGACAGATCCAGTAGTCAATATAAAAATTACTTAGTTCGCATAACTCAGCC
CAAATTAACAGTGCCCACTATGACAATAATTAAAATGGCTAATAAAGCATTAGAAAATTTTACATCACCAACAACGC
CCAATGAAAAAAACATTCCAAATTCAATGATTGCAATTATTGCGGCGGTGGCATTGGGAATGGCACTAATAATAATA
TGCATGTTCCTATATGCTTGTTGCTATAAAAAGTTTCAACATAAACAGGATCCACTACTAAATTTTAACATTTAATT
TTTTATACAGATGTTTTCCACTACAATTTTTATCATTACTAGCCTTGCAGCTGTAACTTATGGCCGTTCACACCTAA
CTCTACCTGTTGGCTCAACATGTACACTACAAGGACCCCAACAAGGCTATGTCACTTGGTGGAGAATATATGATAAT
GGAGGGTTCGCTAGACCATGTGATCAGCCTGGTACAAAATTTTCATGCAACGGAAGAGACTTGACCATAATTAACAT
AACATCAAATGAGCAAGGCTTCTATTATGGAACCAACTATAAAGATAGTTTAGATTACAACATTATTGTAGTGCCAG
CCACCACTTCTGCTCCCCGCAAAACCACTTTCTCTAGCAGCAGTGCCAAAGCAAGCACAATTCCTAAAACAGCTTCT
GCTATGTTAAAGCTTCAAAAAATCGCTTTAAATAATTCCACAGCCGCTCCCAATACAATTCCTAAATCAACAATTGG
CATCATTACTGCCGTGGTAGTGGGATTAATTATTATATTTTTGTGCATAATGTACTATGCCTGCTGCTATAGAAAAC
ATGAACAAAAAGGTGATGCATTACTAAATTTTGACATTTAATTTTTTATAGAATTATGATATTGTTTCAATCAAATA
CCACTAACACTATCAATGTGCAGACTACTTTAAATCATGACATGGAAAACCACACTACCTCCTATGCATACACAAAC
ATTCAGCCTAAATACGCTATGCAATAGAAATTCTAAAAGACGTCCCATCTATTCTCCTATGATTAGTCGTCCCCATA
TGGCTTTGAATGAAATCTAAGATCTTTTTTTTTTTTCTCTTACAGTATGGTGAACACCAATCATGATCCCTAGAAAT
TTCTTCTTCACCATACTCATCTGTGCTTTCAATGTCTGTGCTACTTTCACAGCAGTAGCCACTGCAAGCCCAGACTG
TATAGGACCATTTGCTTCCTATGCACTTTTTGCCTTCGTTACTTGCATCTGCGTGTGTAGCATAGTCTGCCTGGTTA
TTAATTTTTTCCAACTGGTAGACTGGATCTTTGTACGAATTGCCTACCTACGTCACCATCCCGAATACCGCAATCAA
AATGTTGCGGCACTTCTTAGGCTTATTTAAAACCATGCAGGCTATGCTACCAGTCATTTTAATTCTGCTACTACCCT
GCATTGCCCTAGCTTCCACCGCCACTCGCGCTACACCTGAACAACTTAGAAAATGCAAATTTCAACAACCATGGTCA
TTTCTTGATTGCTACCATGAAAAATCTGATTTCCCCACATACTGGATAGTGATTGTTGGAATAATTAACATACTTTC
ATGTACCTTTTTCTCAATCACAATATACCCCACATTTAATTTTGGGTGGAATTCTCCCAATGCACTGGGTTACCCAC
AAGAACCAGATGAACATATCCCACTACAACACATACAACAACCACTAGCACTGGTAGAGTATGAAAATGAGCCACAA
CCTTCACTACCTCCTGCCATTAGTTACTTCAACCTAACCGGCGGAGATGACTGAAATACTCACCACCTCCAATTCCG
CCGAGGATCTGCTTGATATGGACGGCCGCGTCTCAGAACAGCGACTCGCCCAACTACGCATCCGCCAGCAGCAGGAA
CGCGTGACCAAAGAGCTCAGAGATGTCATCCAAATTCACCAATGCAAAAAAGGCATATTTTGCTTGGTAAAACAAGC
CAAGATATCCTACGAGATCACCGCTACTGACCATCGCCTCTCTTACGAACTTGGCCCCCAACGACAAAAATTTACAT
GCATGGTGGGAATCACCCCTATAGTTATCACTCAGCAAAGTGGAGATACTAAGGGGTGCATTCACTGCTCTTGCGAT
TCCATCGAGTGCACCTACACCCTGCTAAAGACCCTATGCGGCCTAAGAGACCTGCTACCCATGAATTAAAAATTAAT
AAAAAATCACTTACTTGAAATCAGCAATAAGGTCTCTGTTGAAATTTTCTCCCAGCAGCACCTCACTTCCCTCTTCC
CAACTCTGGTATTCTAAACCCCGTTCAGCGGCATACTTTCTCCATACTTTAAAGGGGATGTCAAATTTTAACTCCTG
TCCTGTACCCACAATCTTCATGTCTTTCTTCCCAGATGACCAAGAGAGTCCGGCTCAGTGATTCCTTCAACCCTGTC
TACCCCTATGAAGATGAAAGCACCTCCCAACACCCCTTTATAAACCCAGGGTTTATTTCCCCAAATGGCTTTACACA
AAGCCCAGACGGAGTTCTTACTTTAAAATGTTTAACCCCACTAACAACCACAGGCGGGTCTCTACAGTTAAAAGTGG
GAGGGGGTCTTACAATAGATGACACCGACGGTTTTTTGAAAGAAAACATAAGTGCCACCACACCACTCGTTAAGACT
GGTCACTCTATAGGTTTGTCGCTAGGACCCGGATTAGGAACAAATGAAAACAAACTTTGTGCCAAATTGGGAGAAGG
ACTTACATTCAATTCCAACAACATTTGCATTAATGACAATATTAACACCCTATGGACAGGAGTTAACCCCACCAGAG
CCAACTGTCAAATAATGGCCTCCAGTGAATCTAATGATTGCAAATTAATTCTAACACTAGTTAAAACTGGAGCCCTC
GTCACTGCATTTGTTTATGTTATAGGAGTATCTAACGATTTTAATATGCTAACTACACATAAAAATATAAATTTCAC
TGCAGAGCTGTTTTTTGATTCTACTGGTAATTTATTAACTAGCCTTTCATCCCTAAAAACTCCACTTAATCATAAAT
CAGGGCAAAACATGGCTACTGGTGCCCTTACTAATGCTAAAGGTTTCATGCCCAGCACAACTGCCTATCCTTTCAAT
GTTAATTCCAGAGAAAAAGAAAACTACATTTACGGAACTTGTTACTACACAGCTAGTGATCACACTGCTTTTCCCAT
TGACATATCTGTCATGCTTAACCAAAGAGCATTAAATAATGAGACATCATATTGTATTCGTGTAACTTGGTCCTGGA
ATACAGGAGTTGCCCCAGAAGTGCAAACCTCTGCTACTACCCTAGTCACCTCTCCATTTACCTTTTACTACATTAGA
GAAGACGACTGACAAATAAAGTTTAACTTGTTTATTTAAAATCAATTCATAAAATTCGAGTAGTTATTTTGCCTCCC
CCTTCCCATTTAACAGAATACACCAATCTCTCCCCACGCACAGCTTTAAACATTTGGATACCATTACAGATAGACAT
AGTTTTAGATTCCACATTCCAAACAGTTTCAAAGCGAGCCAATCTGGGGTCAGTGATACATAAAAATGCATCGGGAT
AGTCTTTTAAAGCGCTTTCACAGTCCAACTGCTGCGGATGCGACTCCGGAGTCTGGATCACGGTCATCTGGAAGAAG
AACGATGGGAATCATAATCCGAAAACGGAATCGGGCGATTGTGTCTCATCAAACCCACAAGCAGCCGCTGTCTGCGT
CGCTCCGTGCGACTGCTGTTTATAGGATCGGGATCCACAGTGTCCTGAAGCATGATTTTAATAGCCCTTAACATTAA
CTTTCTGGTGCGGTGCGCGCAGCAACGCATTCTGATTTCACTTAGATTACTACAGTAGGTACAGCACATTATCACAA
TATTGTTTAATAAACCATAATTAAAAGCGCTCCAGCCAAAACTCATATCAGATATAATCGCCCCTGCATGACCATCA
TACCAAATTTTAATATAAATTAAATGTCGTTCCCTCAAAAACACACTACCCACATACATAATCTCTTTTGGCATGTG
CATATTAACAATCTGTCTGTACCATGGACAACGTTGGTTAATCATGCAACCCAATATAACCTTCCGAAACCACACTG
CCAACACCGCTCCCCCAGCCATGCATTGAAGTGAACCCTGCCGATTACAATGACAATGAAGAACCCAATTCTCTCGA
CCATGAATCACTTGAGAATAAAAAATATCTATAGTAGCACAACAAAGACATAAATGCATGCATCTTCTCATAATTCT
TAACTCCTCGGGATTTAGAAACATATCCCAAGGAATGGGAAACTCTTGCAGAACAGTAAAGCTGGCAGAACAAGGAA
GACCACGAACACAACTTACACTATGCATAGTCATAGTATCACAATCTGGCAACAGCGGGTGGTCTTCAGTCATAGAA
GCTCGGGTTTCATTTTCCTCACATCGTGGTAACTGGGCTCTGGTGTAAGGGTGATGTCTGGCGCATGATGTCGAGCG
TGCGCGCAACCTTGTCATAATGGAGTTGCTTCCTGACATTCTCGTATTTTGTATAGCAAAACGCGGCCCTGGCACAA
CACACTCTTCTTCGTCTTCTATCCTGCCGCTTAGTGTGTTCCGTCTGATAATTCAAGTACAGCCACACTCTTAAGTT
GGTCAAAAGAATGCTGGCTTCAGTTGTAATCAAAACTCCATCATATTTAATTGTTCTAAGGAAATCATCCACGGTAG
CATATGCAAATCCCAACCAAGCAATGCAACTGGATTGCGTTTCAAGCAGCAGAGGAGAGGGAAGAGACGGAAGAATC
ATGTTAATTTTTATTCCAAACGATCTCGCAGTACTTCAAATTGTAGATCGCGCAGATGGCATCTATCGCCCCCACTG
TGTTGGTGAAAAAGCACAGCTAAATCAAAAGAAATGCGATTTTCAAGGTGCTCAACGGTGGCTTCCAACAAAGCCTC
CACGCGCACATCCAAAAACAAAAGAATACCAAAAGAAGGAGCATTTTCTAACTCCTCAAACATCATATTACATTCCT
GCACCATTCCCAGATAATTTTCAGCTTTCCAGCCTTGAATTATTCGTGTCAGTTCTTGTGGTAAATCCAAACCACAC
ATTACAAACAGGTCCCGGAGGGCGCCCTCCACCACCATTCTTAAACACACCCTCATAATGTCAAAATATCTTGCTCC
TGTGTCACCTGTAGCAAATTAAGAATGGCATCATCAATTGACATGCCCTTGGCTCTAAGTTCTTCTCTAAGTTCTAG
TTGTAAATACTCTTTCATATTATCACCAAACTGCTTAGCCAGAAGCCCCCCGGGAACAATAGCAGGGGACGCTACAG
TGCAGTACAAGCGCAGACCTCCCCAATTGGCTCCAGCAAAAACAAGATTAGAATAAGCATACTGGGAACCACCAGTA
ATATCATCAAAGTTGCTGGAAATATAATCAGGCAGAGTTTCTTGTAAAAATTGAATAAAAGAAAAATTTTCCAAAGA
AACATTCAAAACCTCTGGGATGCAAATGCAATAGGTTACCGCGCTGCGCTCCAACATTATTAGTTTTGAATTAGTCT
GTAAAATAAAAGAAACAAGCGTCATATCATAGTAGCCTGTCGAACAGGTGGATAAATCAGTCTTTCCATTACAAGAC
AAGCCACAGGGTCTCCAGCTCGACCCTCGTAAAACCTGTCATCGTGATTAAACAACAGCACCGAAAGTTCCTCGCGG
TGGCCAGCATGAATAATTCTTGATGAAGCATATAATCCAGACATGTTAGCATCAGTTAAAGAAAAAAAACAGCCAAC
ATAGCCTCTGGGTATAATTATGCTTAATCTTAAGTATAGCAAAGCCACCCCTCGCGGATACAAAGTAAAAGGCACAG
GAGAATAAAAAATATAATTATTTCTCTGCTGCTGTTCAGGCAACGTTGCCCCCGGTCCCTCTAAATACACATACAAA
GCCTCATCAGCCATGGCTTACCAGACAAAGTATAGCGGGCGCACAAAGCACAAGCTCTAAAGAAGCTCTAAAGACGC
TCTCCAACCTCTCCACAATATATACACAAGCCCTAAACTGACGTAATGGGAGTAAAGTGTAAAAAATCCCGCCAAGC
CCAACACACACCCCGAAACTGCGTCAGCAGGGAAAAGTACAGTTTCACTTCCGCAAACCCAACAAGCGTAGCTTCCT
CTTTCTCACGGTACGTCACATCCGATTAACTTGCAACGTCATTTTCCCACGGCCGCCCCGCCCATTTTAGCCGTTAA
CCCCACAGCCAATCACCACACAGCGCGCACTTTTTTAAATTACCTCATTTACATATTGGCACCATTCCATCTATAAG
GTATATTATATAGAGAG

GenBank Accession No. AP_000564 (SEQ ID NO: 217)

MTKRVRLSDSFNPVYPYEDESTSQHPFINPGFISPNGFTQSPDGVLTLKC
LTPLITTGGSLQLKVGGGLTIDDTDGFLKENISATTPLVKTGHSIGLSLG
PGLGTNENKLCAKLGEGLTENSNNICINDNINTLWTGVNPTRANCQIMAS
SESNDCKLILTLVKTGALVTAFVYVIGVSNDFNMLTTHKNINFTAELFFD
STGNLLTSLSSLKTPLNHKSGQNMATGALTNAKGFMPSTTAYPFNVNSRE
KENYIYGTCYYTASDHTAFPIDISVMLNQRALNNETSYCIRVTWSWNTGV
APEVQTSATTLVTSPFTFYYIREDD

GenBank Accession No. AP 000543 (SEQ ID NO: 218)

MRRRAVLGGAVVYPEGPPPSYESVMQQQAAMIQPPLEVPFVPPRYLAPTE
GRNSIRYSELSPLYDTTKLYLVDNKSADIASLNYQNDHSNFLTTVVQNND
FTPTEASTQTINFDERSRWGGHLKTIMHTNMPNVNEYMFSNKFKARVMVS
RKAPEGVTVNDTYDHKEDILKYEWFEFILPEGNFSATMTIDLMNNAIIDN
YLEIGRQNGVLESDIGVKFDTRNFRLGWDPETKLIMPGVYTYEAFHPDIV
LLPGCGVDFTESRLSNLLGIRKRHPFQEGFKIMYEDLEGGNIPALLDVTA
YEESKKDTTTETTTLAVAEETSEDDNITRGDTYITEKHKREAAAAEVKKE
LKIQPLEKDSKSRSYNVLEDKINTAYRSWYLSYNYGNPKKGIRSWTLLTT
SDVTCGAEQVYWSLPDMMQDPVTFRSTRQVNNYPVVGAELMPVFSKSFYN
EQAVYSQQLRQATSLTHVENRFPENQILIRPPAPTITTVSENVPALTDHG
TLPLRSSIRGVQRVTVTDARRRTCPYVYKALGIVAPRVLSSRTF

GenBank Accession No. AP_000548 (SEQ ID NO: 219)

MATPSMMPQWAYMHIAGQDASEYLSPGLVQFARATDTYFSMGNKFRNPTV
APTHDVTTDRSQRLMLRFVPVDREDNTYSYKVRYTLAVGDNRVLDMASTF
FDIRGVLDRGPSFKPYSGTAYNSLAPKGAPNTSQWIVTTGEDNATTYTFG
IASTKGDNITKEGLEIGKDITADNKPIYADKTYQPEPQVGEESWTDIDGT
NEKFGGRALKPATKMKPCYGSFARPTNIKGGQAKNRKVTPTEGDVEAEEP
DIDMEFFDGREAADAFSPEIVLYTENVNLETPDSHVVYKPGTSDGNSHAN
LGQQAMPNRPNYIGFRDNFVGLMYYNSTGNMGVLAGQASQLNAVVDLQDR
NTELSYQLLLDSLGDRSRYFSMWNQAVDSYDPDVRIIENHGVEDELPNYC
FPLDGIGPGNKYQGIKPRDTAWEKDTKVSTANEIAIGNNLAMEINIQANL
WRSFLYSNVALYLPDVYKYTPTNITLPANTNTYEYMNGRVVSPSLVDSYI
NIGARWSLDPMDNVNPFNHHRNAGLRYRSMLLGNGRYVPFHIQVPQKFFA
VKNLLLLPGSYTYEWNFRKDVNMVLQSSLGNDLRTDGANISFTSINLYAT
FFPMAHNTASTLEAMLRNDTNDQSENDYLSAANMLYPIPANATNIPISIP
SRNWAAFRGWSFTRLKTKETPSLGSGFDPYFVYSGSIPYLDGTFYLNHTF
KKVSIMFDSSVSWPGNDRLLSPNEFEIKRTVDGEGYNVAQCNMTKDWFLV
QMLANYNIGYQGFYIPEGYKDRMYSFFRNFQPMSRQVVDEVNYTDYKAVT
LPYQHNNSGFVGYLAPTMRQGEPYPANYPYPLIGTTAVKSVTQKKFLCDR
TMWRIPFSSNFMSMGALTDLGQNLLYANSAHALDMTFEVDPMDEPTLLYL
LFEVEDVVRVHQPHRGVIEAVYLRTPFSAGNATT

GenBank Accession No. NC_011202 (SEQ ID NO: 201)

CATCATCAATAATATACCTTATAGATGGAATGGTGCCAATATGTAAATGAGGTGATTTTAAAAAGTGTGGATCGTGT
GGTGATTGGCTGTGGGGTTAACGGCTAAAAGGGGCGGTGCGACCGTGGGAAAATGACGTTTTGTGGGGGTGGAGTTT
TTTTGCAAGTIGTCGCGGGAAATGTGACGCATAAAAAGGCTTTTTTCTCACGGAACTACTTAGTTTTCCCACGGTAT
TTAACAGGAAATGAGGTAGTTTTGACCGGATGCAAGTGAAAATTGTTGATTTTCGCGCGAAAACTGAATGAGGAAGT
GTTTTTCTGAATAATGTGGTATTTATGGCAGGGTGGAGTATTTGTTCAGGGCCAGGTAGACTTTGACCCATTACGTG
GAGGTTTCGATTACCGTGTTTTTTACCTGAATTTCCGCGTACCGTGTCAAAGTCTTCTGTTTTTACGTAGGTGTCAG
CTGATCGCTAGGGTATTTATACCTCAGGGTTTGTGTCAAGAGGCCACTCTTGAGTGCCAGCGAGAAGAGTTTTCTCC
TCTGCGCCGGCAGTTTAATAATAAAAAAATGAGAGATTTGCGATTTCTGCCTCAGGAAATAATCTCTGCTGAGACTG
GAAATGAAATATTGGAGCTTGTGGTGCACGCCCTGATGGGAGACGATCCGGAGCCACCTGTGCAGCTTTTTGAGCCT
CCTACGCTTCAGGAACTGTATGATTTAGAGGTAGAGGGATCGGAGGATTCTAATGAGGAAGCTGTAAATGGCTTTTT
TACCGATTCTATGCTTTTAGCTGCTAATGAAGGGTTAGAATTAGATCCGCCTTTGGACACTTTTGATACTCCAGGGG
TAATTGTGGAAAGCGGTACAGGTGTAAGAAAATTACCTGATTTGAGTTCCGTGGACTGTGATTTGCACTGCTATGAA
GACGGGTTTCCTCCGAGTGATGAGGAGGACCATGAAAAGGAGCAGTCCATGCAGACTGCAGCGGGTGAGGGAGTGAA
GGCTGCCAATGTTGGTTTTCAGTTGGATTGCCCGGAGCTTCCTGGACATGGCTGTAAGTCTTGTGAATTTCACAGGA
AAAATACTGGAGTAAAGGAACTGTTATGTTCGCTTTGTTATATGAGAACGCACTGCCACTTTATTTACAGTAAGTGT
GTTTAAGTTAAAATTTAAAGGAATATGCTGTTTTTCACATGTATATTGAGTGTGAGTTTTGTGCTTCTTATTATAGG
TCCTGTGTCTGATGCTGATGAATCACCATCTCCTGATTCTACTACCTCACCTCCTGAGATTCAAGCACCTGTTCCTG
TGGACGTGCGCAAGCCCATTCCTGTGAAGCTTAAGCCTGGGAAACGTCCAGCAGTGGAAAAACTTGAGGACTTGTTA
CAGGGTGGGGACGGACCTTTGGACTTGAGTACACGGAAACGTCCAAGACAATAAGTGTTCCATATCCGTGTTTACTT
AAGGTGACGTCAATATTTGTGTGACAGTGCAATGTAATAAAAATATGTTAACTGTTCACTGGTTTTTATTGCTTTTT
GGGCGGGGACTCAGGTATATAAGTAGAAGCAGACCTGTGTGGTTAGCTCATAGGAGCTGGCTTTCATCCATGGAGGT
TTGGGCCATTTTGGAAGACCTTAGGAAGACTAGGCAACTGTTAGAGAACGCTTCGGACGGAGTCTCCGGTTTTTGGA
GATTCTGGTTCGCTAGTGAATTAGCTAGGGTAGTTTTTAGGATAAAACAGGACTATAACCAAGAATTTGAAAAGTTG
TTGGTAGATTGCCCAGGACTTTTTGAAGCTCTTAATTTGGGCCATCAGGTTCACTTTAAAGAAAAAGTTTTATCAGT
TTTAGACTTTTCAACCCCAGGTAGAACTGCTGCTGCTGTGGCTTTTCTTACTTTTATATTAGATAAATGGATCCCGC
AGACTCATTTCAGCAGGGGATACGTTTTGGATTTCATAGCCACAGCATTGTGGAGAACATGGAAGGTTCGCAAGATG
AGGACAATCTTAGGTTACTGGCCAGTGCAGCCTTTGGGTGTAGCGGGAATCCTGAGGCATCCACCGGTCATGCCAGC
GGTTCTGGAGGAGGAACAGCAAGAGGACAACCCGAGAGCCGGCCTGGACCCTCCAGTGGAGGAGGCGGAGTAGCTGA
CTTGTCTCCTGAACTGCAACGGGTGCTTACTGGATCTACGTCCACTGGACGGGATAGGGGCGTTAAGAGGGAGAGGG
CATCTAGTGGTACTGATGCTAGATCTGAGTTGGCTTTAAGTTTAATGAGTCGCAGACGTCCTGAAACCATTIGGTGG
CATGAGGTTCAGAAAGAGGGAAGGGATGAAGTTTCTGTATTGCAGGAGAAATATTCACTGGAACAGGTGAAAACATG
TTGGTTGGAGCCTGAGGATGATTGGGAGGTGGCCATTAAAAATTATGCCAAGATAGCTTTGAGGCCTGATAAACAGT
ATAAGATTACTAGACGGATTAATATCCGGAATGCTTGTTACATATCTGGAAATGGGGCTGAGGTGGTAATAGATACT
CAAGACAAGGCAGTTATTAGATGCTGCATGATGGATATGTGGCCTGGGGTAGTCGGTATGGAAGCAGTAACTTTTGT
AAATGTTAAGTTTAGGGGAGATGGTTATAATGGAATAGTGTTTATGGCCAATACCAAACTTATATTGCATGGTTGTA
GCTTTTTTGGTTTCAACAATACCTGTGTAGATGCCTGGGGACAGGTTAGTGTACGGGGATGTAGTTTCTATGCGTGT
TGGATTGCCACAGCTGGCAGAACCAAGAGTCAATTGTCTCTGAAGAAATGCATATTTCAAAGATGTAACCTGGGCAT
TCTGAATGAAGGCGAAGCAAGGGTCCGCCACTGCGCTTCTACAGATACTGGATGTTTTATTTTGATTAAGGGAAATG
CCAGCGTAAAGCATAACATGATTTGCGGTGCTTCCGATGAGAGGCCTTATCAAATGCTCACTTGTGCTGGTGGGCAT
TGTAATATGCTGGCTACTGTGCATATTGTTTCCCATCAACGCAAAAAATGGCCTGTTTTTGATCACAATGTGATGAC
GAAGTGTACCATGCATGCAGGTGGGCGTAGAGGAATGTTTATGCCTTACCAGTGTAACATGAATCATGTGAAAGTGT
TGTTGGAACCAGATGCCTTTTCCAGAATGAGCCTAACAGGAATTTTTGACATGAACATGCAAATCTGGAAGATCCTG
AGGTATGATGATACGAGATCGAGGGTACGCGCATGCGAATGCGGAGGCAAGCATGCCAGGTTCCAGCCGGTGTGTGT
AGATGTGACTGAAGATCTCAGACCGGATCATTTGGTTATTGCCCGCACTGGAGCAGAGTTCGGATCCAGTGGAGAAG
AAACTGACTAAGGTGAGTATTGGGAAAACTTTGGGGTGGGATTTTCAGATGGACAGATTGAGTAAAAATTTGTTTTT
TCTGTCTTGCAGCTGTCATGAGTGGAAACGCTTCTTTTAAGGGGGGAGTCTTCAGCCCTTATCTGACAGGGCGTCTC
CCATCCTGGGCAGGAGTTCGTCAGAATGTTATGGGATCTACTGTGGATGGAAGACCCGTCCAACCCGCCAATTCTTC
AACGCTGACCTATGCTACTTTAAGTTCTTCACCTTTGGACGCAGCTGCAGCTGCCGCCGCCGCTTCTGTTGCCGCTA
ACACTGTGCTTGGAATGGGTTACTATGGAAGCATCATGGCTAATTCCACTTCCTCTAATAACCCTTCTACCCTGACT
CAGGACAAGTTACTTGTCCTTTTGGCCCAGCTGGAGGCTTTGACCCAACGTCTGGGTGAACTTTCTCAGCAGGTGGT
CGAGTTGCGAGTACAAACTGAGTCTGCTGTCGGCACGGCAAAGTCTAAATAAAAAAATCCCAGAATCAATGAATAAA
TAAACAAGCTTGTTGTTGATTTAAAATCAAGTGTTTTTATTTCATTTTTCGCGCACGGTATGCCCTAGACCACCGAT
CTCTATCATTGAGAACTCGGTGGATTTTTTCCAGGATCCTATAGAGGTGGGATTGAATGTTTAGATACATGGGCATT
AGGCCGTCTTTGGGGTGGAGATAGCTCCATTGAAGGGATTCATGCTCCGGGGTAGTGTTGTAAATCACCCAGTCATA
ACAAGGTCGCAGTGCATGGTGTTGCACAATATCTTTTAGAAGTAGGCTGATTGCCACAGATAAGCCCTTGGTGTAGG
TGTTTACAAACCGGTTGAGCTGGGATGGGTGCATTCGGGGTGAAATTATGTGCATTTTGGATTGGATTTTTAAGTTG
GCAATATTGCCGCCAAGATCCCGTCTTGGGTTCATGTTATGAAGGACCACCAAGACGGTGTATCCGGTACATTTAGG
AAATTTATCGTGCAGCTTGGATGGAAAAGCGTGGAAAAATTTGGAGACACCCTTGTGTCCTCCAAGATTTTCCATGC
ACTCATCCATGATAATAGCAATGGGGCCGTGGGCAGCGGCGCGGGCAAACACGTTCCGTGGGTCTGACACATCATAG
TTATGTTCCTGAGTTAAATCATCATAAGCCATTTTAATGAATTTGGGGCGGAGAGTACCAGATTGGGGTATGAATGT
TCCTTCGGGCCCCGGAGCATAGTTCCCCTCACAGATTTGCATTTCCCAAGCTTTCAGTTCCGAGGGTGGAATCATGT
CCACCTGGGGGGCTATGAAAAACACCGTTTCTGGGGCGGGGGTGATTAATTGTGATGATAGCAAATTTCTGAGCAAT
TGAGATTTGCCACATCCGGTGGGGCCATAAATGATTCCGATTACGGGTTGCAGGTGGTAGTTTAGGGAACGGCAACT
GCCGTCTTCTCGAAGCAAGGGGGCCACCTCGTTCATCATTTCCCTTACATGCATATTTTCCCGCACCAAATCCATTA
GGAGGCGCTCTCCTCCTAGTGATAGAAGTTCTTGTAGTGAGGAAAAGTTTTTCAGCGGTTTCAGACCGTCAGCCATG
GGCATTTTGGAGAGAGTTTGCTGCAAAAGTTCTAGTCTGTTCCACAGTTCAGTGATGTGTTCTATGGCATCTCGATC
CAGCAGACCTCCTCGTTTCGCGGGTTTGGACGGCTCCTGGAATAGGGTATGAGACGATGGGCGTCCAGCGCTGCCAG
GGTTCGGTCCTTCCAGGGTCTCAGTGTTCGAGTCAGGGTTGTTTCCGTCACAGTGAAGGGGTGTGCGCCTGCTTGGG
CGCTTGCCAGGGTGCGCTTCAGACTCATCCTGCTGGTCGAAAACTTCTGTCGCTTGGCGCCCTGTATGTCGGCCAAG
TAGCAGTTTACCATGAGTTCGTAGTTGAGCGCCTCGGCTGCGTGGCCTTTGGCGCGGAGCTTACCTTTGGAAGTTTT
CTTGCATACCGGGCAGTATAGGCATTTCAGCGCATACAACTTGGGCGCAAGGAAAACGGATTCTGGGGAGTATGCAT
CTGCGCCGCAGGAGGCGCAAACAGTTTCACATTCCACCAGCCAGGTTAAATCCGGTTCATTGGGGTCAAAAACAAGT
TTTCCGCCATATTTTTTGATGCGTTTCTTACCTTTGGTCTCCATGAGTTCGTGTCCTCGTTGAGTGACAAACAGGCT
GTCCGTGTCCCCGTAGACTGATTTTACAGGCCTCTTCTCCAGTGGAGTGCCTCGGTCTTCTTCGTACAGGAACTCTG
ACCACTCTGATACAAAGGCGCGCGTCCAGGCCAGCACAAAGGAGGCTATGTGGGAGGGGTAGCGATCGTTGTCAACC
AGGGGGTCCACCTTTTCCAAAGTATGCAAACACATGTCACCCTCTTCAACATCCAGGAATGTGATTGGCTTGTAGGT
GTATTTCACGTGACCTGGGGTCCCCGCTGGGGGGGTATAAAAGGGGGCGGTTCTTTGCTCTTCCTCACTGTCTTCCG
GATCGCTGTCCAGGAACGTCAGCTGTTGGGGTAGGTATTCCCTCTCGAAGGCGGGCATGACCTCTGCACTCAGGTTG
TCAGTTTCTAAGAACGAGGAGGATTTGATATTGACAGTGCCGGTTGAGATGCCTTTCATGAGGTTTTCGTCCATTTG
GTCAGAAAACACAATTTTTTTATTGTCAAGTTTGGTGGCAAATGATCCATACAGGGCGTTGGATAAAAGTTTGGCAA
TGGATCGCATGGTTTGGTTCTTTTCCTTGTCCGCGCGCTCTTTGGCGGCGATGTTGAGTTGGACATACTCGCGTGCC
AGGCACTTCCATTCGGGGAAGATAGTTGTTAATTCATCTGGCACGATTCTCACTTGCCACCCTCGATTATGCAAGGT
AATTAAATCCACACTGGTGGCCACCTCGCCTCGAAGGGGTTCATTGGTCCAACAGAGCCTACCTCCTTTCCTAGAAC
AGAAAGGGGGAAGTGGGTCTAGCATAAGTTCATCGGGAGGGTCTGCATCCATGGTAAAGATTCCCGGAAGTAAATCC
TTATCAAAATAGCTGATGGGAGTGGGGTCATCTAAGGCCATTTGCCATTCTCGAGCTGCCAGTGCGCGCTCATATGG
GTTAAGGGGACTGCCCCATGGCATGGGATGGGTGAGTGCAGAGGCATACATGCCACAGATGTCATAGACGTAGATGG
GATCCTCAAAGATGCCTATGTAGGTTGGATAGCATCGCCCCCCTCTGATACTTGCTCGCACATAGTCATATAGTTCA
TGTGATGGCGCTAGCAGCCCCGGACCCAAGTTGGTGCGATTGGGTTTTTCTGTTCTGTAGACGATCTGGCGAAAGAT
GGCGTGAGAATTGGAAGAGATGGTGGGTCTTTGAAAAATGTTGAAATGGGCATGAGGTAGACCTACAGAGTCTCTGA
CAAAGTGGGCATAAGATTCTTGAAGCTTGGTTACCAGTTCGGCGGTGACAAGTACGTCTAGGGCGCAGTAGTCAAGT
GTTTCTTGAATGATGTCATAACCTGGTTGGTTTTTCTTTTCCCACAGTTCGCGGTTGAGAAGGTATTCTTCGCGATC
CTTCCAGTACTCTTCTAGCGGAAACCCGTCTTTGTCTGCACGGTAAGATCCTAGCATGTAGAACTGATTAACTGCCT
TGTAAGGGCAGCAGCCCTTCTCTACGGGTAGAGAGTATGCTTGAGCAGCTTTTCGTAGCGAAGCGTGAGTAAGGGCA
AAGGTGTCTCTGACCATGACTTTGAGAAATTGGTATTTGAAGTCGATGTCGTCACAGGCTCCCTGTTCCCAGAGTTG
GAAGTCTACCCGTTTCTTGTAGGCGGGGTTGGGCAAAGCGAAAGTAACATCATTGAAGAGAATCTTACCGGCTCTGG
GCATAAAATTGCGAGTGATGCGAAAAGGCTGTGGTACTTCCGCTCGATTGTTGATCACCTGGGCAGCTAGGACGATC
TCGTCGAAACCGTTGATGTTGTGTCCTACGATGTATAATTCTATGAAACGCGGCGTGCCTCTGACGTGAGGTAGCTT
ACTGAGCTCATCAAAGGTTAGGTCTGTGGGGTCAGATAAGGCGTAGTGTTCGAGAGCCCATTCGTGCAGGTGAGGAT
TTGCATGTAGGAATGATGACCAAAGATCTACCGCCAGTGCTGTTTGTAACTGGTCCCGATACTGACGAAAATGCCGG
CCAATTGCCATTTTTTCTGGAGTGACACAGTAGAAGGTTCTGGGGTCTTGTTGCCATCGATCCCACTTGAGTTTAAT
GGCTAGATCGTGGGCCATGTTGACGAGACGCTCTTCTCCTGAGAGTTTCATGACCAGCATGAAAGGAACTAGTTGTT
TGCCAAAGGATCCCATCCAGGTGTAAGTTTCCACATCGTAGGTCAGGAAGAGTCTTTCTGTGCGAGGATGAGAGCCG
ATCGGGAAGAACTGGATTTCCTGCCACCAGTTGGAGGATTGGCTGTTGATGTGATGGAAGTAGAAGTTTCTGCGGCG
CGCCGAGCATTCGTGTTTGTGCTTGTACAGACGGCCGCAGTAGTCGCAGCGTTGCACGGGTTGTATCTCGTGAATGA
GTTGTACCTGGCTTCCCTTGACGAGAAATTTCAGTGGGAAGCCGAGGCCTGGCGATTGTATCTCGTGCTCTTCTATA
TTCGCTGTATCGGCCTGTTCATCTTCTGTTTCGATGGTGGTCATGCTGACGAGCCCCCGCGGGAGGCAAGTCCAGAC
CTCGGCGCGGGAGGGGCGGAGCTGAAGGACGAGAGCGCGCAGGCTGGAGCTGTCCAGAGTCCTGAGACGCTGCGGAC
TCAGGTTAGTAGGTAGGGACAGAAGATTAACTTGCATGATCTTTTCCAGGGCGTGCGGGAGGTTCAGATGGTACTTG
ATTTCCACAGGTTCGTTTGTAGAGACGTCAATGGCTTGCAGGGTTCCGTGTCCTTTGGGCGCCACTACCGTACCTTT
GTTTTTTCTTTTGATCGGTGGTGGCTCTCTTGCTTCTTGCATGCTCAGAAGCGGTGACGGGGACGCGCGCCGGGCGG
CAGCGGTTGTTCCGGACCCGAGGGCATGGCTGGTAGTGGCACGTCGGCGCCGCGCACGGGCAGGTTCTGGTACTGCG
CTCTGAGAAGACTTGCGTGCGCCACCACGCGTCGATTGACGTCTTGTATCTGACGTCTCTGGGTGAAAGCTACCGGC
CCCGTGAGCTTGAACCTGAAAGAGAGTTCAACAGAATCAATTTCGGTATCGTTAACGGCAGCTTGTCTCAGTATTTC
TTGTACGTCACCAGAGTTGTCCTGGTAGGCGATCTCCGCCATGAACTGCTCGATTTCTTCCTCCTGAAGATCTCCGC
GACCCGCTCTTTCGACGGTGGCCGCGAGGTCATTGGAGATACGGCCCATGAGTTGGGAGAATGCATTCATGCCCGCC
TCGTTCCAGACGCGGCTGTAAACCACGGCCCCCTCGGAGTCTCTTGCGCGCATCACCACCTGAGCGAGGTTAAGCTC
CACGTGTCTGGTGAAGACCGCATAGTTGCATAGGCGCTGAAAAAGGTAGTTGAGTGTGGTGGCAATGTGTTCGGCGA
CGAAGAAATACATGATCCATCGTCTCAGCGGCATTTCGCTAACATCGCCCAGAGCTTCCAAGCGCTCCATGGCCTCG
TAGAAGTCCACGGCAAAATTAAAAAACTGGGAGTTTCGCGCGGACACGGTCAATTCCTCCTCGAGAAGACGGATGAG
TTCGGCTATGGTGGCCCGTACTTCGCGTTCGAAGGCTCCCGGGATCTCTTCTTCCTCTTCTATCTCTTCTTCCACTA
ACATCTCTTCTTCGTCTTCAGGCGGGGGCGGAGGGGGCACGCGGCGACGTCGACGGCGCACGGGCAAACGGTCGATG
AATCGTTCAATGACCTCTCCGCGGCGGCGGCGCATGGTTTCAGTGACGGCGCGGCCGTTCTCGCGCGGTCGCAGAGT
AAAAACACCGCCGCGCATCTCCTTAAAGTGGTGACTGGGAGGTTCTCCGTTTGGGAGGGAGAGGGCGCTGATTATAC
ATTTTATTAATTGGCCCGTAGGGACTGCACGCAGAGATCTGATCGTGTCAAGATCCACGGGATCTGAAAACCTTTCG
ACGAAAGCGTCTAACCAGTCACAGTCACAAGGTAGGCTGAGTACGGCTTCTTGTGGGCGGGGGTGGTTATGTGTTCG
GTCTGGGTCTTCTGTTTCTTCTTCATCTCGGGAAGGTGAGACGATGCTGCTGGTGATGAAATTAAAGTAGGCAGTTC
TAAGACGGCGGATGGTGGCGAGGAGCACCAGGTCTTTGGGTCCGGCTTGCTGGATACGCAGGCGATTGGCCATTCCC
CAAGCATTATCCTGACATCTAGCAAGATCTTTGTAGTAGTCTTGCATGAGCCGTTCTACGGGCACTTCTTCCTCACC
CGTTCTGCCATGCATACGTGTGAGTCCAAATCCGCGCATTGGTTGTACCAGTGCCAAGTCAGCTACGACTCTTTCGG
CGAGGATGGCTTGCTGTACTTGGGTAAGGGTGGCTTGAAAGTCATCAAAATCCACAAAGCGGTGGTAAGCTCCTGTA
TTAATGGTGTAAGCACAGTTGGCCATGACTGACCAGTTAACTGTCTGGTGACCAGGGCGCACGAGCTCGGTGTATTT
AAGGCGCGAATAGGCGCGGGTGTCAAAGATGTAATCGTTGCAGGTGCGCACCAGATACTGGTACCCTATAAGAAAAT
GCGGCGGTGGTTGGCGGTAGAGAGGCCATCGTTCTGTAGCTGGAGCGCCAGGGGCGAGGTCTTCCAACATAAGGCGG
TGATAGCCGTAGATGTACCTGGACATCCAGGTGATTCCTGCGGCGGTAGTAGAAGCCCGAGGAAACTCGCGTACGCG
GTTCCAAATGTTGCGTAGCGGCATGAAGTAGTTCATTGTAGGCACGGTTTGACCAGTGAGGCGCGCGCAGTCATTGA
TGCTCTATAGACACGGAGAAAATGAAAGCGTTCAGCGACTCGACTCCGTAGCCTGGAGGAACGTGAACGGGTTGGGT
CGCGGTGTACCCCGGTTCGAGACTTGTACTCGAGCCGGCCGGAGCCGCGGCTAACGTGGTATTGGCACTCCCGTCTC
GACCCAGCCTACAAAAATCCAGGATACGGAATCGAGTCGTTTTGCTGGTTTCCGAATGGCAGGGAAGTGAGTCCTAT
TTTTTTTTTTTGCCGCTCAGATGCATCCCGTGCTGCGACAGATGCGCCCCCAACAACAGCCCCCCTCGCAGCAGCAG
CAGCAGCAATCACAAAAGGCTGTCCCTGCAACTACTGCAACTGCCGCCGTGAGCGGTGCGGGACAGCCCGCCTATGA
TCTGGACTTGGAAGAGGGCGAAGGACTGGCACGTCTAGGTGCGCCTTCACCCGAGCGGCATCCGCGAGTTCAACTGA
AAAAAGATTCTCGCGAGGCGTATGTGCCCCAACAGAACCTATTTAGAGACAGAAGCGGCGAGGAGCCGGAGGAGATG
CGAGCTTCCCGCTTTAACGCGGGTCGTGAGCTGCGTCACGGTTTGGACCGAAGACGAGTGTTGCGGGACGAGGATTT
CGAAGTTGATGAAATGACAGGGATCAGTCCTGCCAGGGCACACGTGGCTGCAGCCAACCTTGTATCGGCTTACGAGC
AGACAGTAAAGGAAGAGCGTAACTTCCAAAAGTCTTTTAATAATCATGTGCGAACCCTGATTGCCCGCGAAGAAGTT
ACCCTTGGTTTGATGCATTTGTGGGATTTGATGGAAGCTATCATTCAGAACCCTACTAGCAAACCTCTGACCGCCCA
GCTGTTTCTGGTGGTGCAACACAGCAGAGACAATGAGGCTTTCAGAGAGGCGCTGCTGAACATCACCGAACCCGAGG
GGAGATGGTTGTATGATCTTATCAACATTCTACAGAGTATCATAGTGCAGGAGCGGAGCCTGGGCCTGGCCGAGAAG
GTGGCTGCCATCAATTACTCGGTTTTGAGCTTGGGAAAATATTACGCTCGCAAAATCTACAAGACTCCATACGTTCC
CATAGACAAGGAGGTGAAGATAGATGGGTTCTACATGCGCATGACGCTCAAGGTCTTGACCCTGAGCGATGATCTTG
GGGTGTATCGCAATGACAGAATGCATCGCGCGGTTAGCGCCAGCAGGAGGCGCGAGTTAAGCGACAGGGAACTGATG
CACAGTTTGCAAAGAGCTCTGACTGGAGCTGGAACCGAGGGTGAGAATTACTTCGACATGGGAGCTGACTTGCAGTG
GCAGCCTAGTCGCAGGGCTCTGAGCGCCGCGACGGCAGGATGTGAGCTTCCTTACATAGAAGAGGCGGATGAAGGCG
AGGAGGAAGAGGGCGAGTACTTGGAAGACTGATGGCACAACCCGTGTTTTTTGCTAGATGGAACAGCAAGCACCGGA
TCCCGCAATGCGGGCGGCGCTGCAGAGCCAGCCGTCCGGCATTAACTCCTCGGACGATTGGACCCAGGCCATGCAAC
GTATCATGGCGTTGACGACTCGCAACCCCGAAGCCTTTAGACAGCAACCCCAGGCCAACCGTCTATCGGCCATCATG
GAAGCTGTAGTGCCTTCCCGCTCTAATCCCACTCATGAGAAGGTCCTGGCCATCGTGAACGCGTTGGTGGAGAACAA
AGCTATTCGTCCAGATGAGGCCGGACTGGTATACAACGCTCTCTTAGAACGCGTGGCTCGCTACAACAGTAGCAATG
TGCAAACCAATTTGGACCGTATGATAACAGATGTACGCGAAGCCGTGTCTCAGCGCGAAAGGTTCCAGCGTGATGCC
AACCTGGGTTCGCTGGTGGCGTTAAATGCTTTCTTGAGTACTCAGCCTGCTAATGTGCCGCGTGGTCAACAGGATTA
TACTAACTTTTTAAGCGCTTTGAGACTGATGGTATCAGAAGTACCTCAGAGCGAAGTGTATCAGTCCGGTCCTGATT
ACTTCTTTCAGACTAGCAGACAGGGCTTGCAGACGGTAAATCTGAGCCAAGCTTTTAAAAACCTTAAAGGTTTGTGG
GGAGTGCATGCCCCGGTAGGAGAAAGAGCAACCGTGTCTAGCTTGTTAACTCCGAACTCCCGCCTATTATTACTGTT
GGTAGCTCCTTTCACCGACAGCGGTAGCATCGACCGTAATTCCTATTTGGGTTACCTACTAAACCTGTATCGCGAAG
CCATAGGGCAAAGTCAGGTGGACGAGCAGACCTATCAAGAAATTACCCAAGTCAGTCGCGCTTTGGGACAGGAAGAC
ACTGGCAGTTTGGAAGCCACTCTGAACTTCTTGCTTACCAATCGGTCTCAAAAGATCCCTCCTCAATATGCTCTTAC
TGCGGAGGAGGAGAGGATCCTTAGATATGTGCAGCAGAGCGTGGGATTGTTTCTGATGCAAGAGGGGGCAACTCCGA
CTGCAGCACTGGACATGACAGCGCGAAATATGGAGCCCAGCATGTATGCCAGTAACCGACCTTTCATTAACAAACTG
CTGGACTACTTGCACAGAGCTGCCGCTATGAACTCTGATTATTTCACCAATGCCATCTTAAACCCGCACTGGCTGCC
CCCACCTGGTTTCTACACGGGCGAATATGACATGCCCGACCCTAATGACGGATTTCTGTGGGACGACGTGGACAGCG
ATGTTTTTTCACCTCTTTCTGATCATCGCACGTGGAAAAAGGAAGGCGGCGATAGAATGCATTCTTCTGCATCGCTG
TCCGGGGTCATGGGTGCTACCGCGGCTGAGCCCGAGTCTGCAAGTCCTTTTCCTAGTCTACCCTTTTCTCTACACAG
TGTACGTAGCAGCGAAGTGGGTAGAATAAGTCGCCCGAGTTTAATGGGCGAAGAGGAGTATCTAAACGATTCCTTGC
TCAGACCGGCAAGAGAAAAAAATTTCCCAAACAATGGAATAGAAAGTTTGGTGGATAAAATGAGTAGATGGAAGACT
TATGCTCAGGATCACAGAGACGAGCCTGGGATCATGGGGATTACAAGTAGAGCGAGCCGTAGACGCCAGCGCCATGA
CAGACAGAGGGGTCTTGTGTGGGACGATGAGGATTCGGCCGATGATAGCAGCGTGCTGGACTTGGGTGGGAGAGGAA
GGGGCAACCCGTTTGCTCATTTGCGCCCTCGCTTGGGTGGTATGTTGTAAAAAAAAATAAAAAAAAAACTCACCAAG
GCCATGGCGACGAGCGTACGTTCGTTCTTCTTTATTATCTGTGTCTAGTATAATGAGGCGAGTCGTGCTAGGCGGAG
CGGTGGTGTATCCGGAGGGTCCTCCTCCTTCGTACGAGAGCGTGATGCAGCAGCAGCAGGCGACGGCGGTGATGCAA
TCCCCACTGGAGGCTCCCTTTGTGCCTCCGCGATACCTGGCACCTACGGAGGGCAGAAACAGCATTCGTTATTCGGA
ACTGGCACCTCAGTACGATACCACCAGGTTGTATCTGGTGGACAACAAGTCGGCGGACATTGCTTCTCTGAACTATC
AGAATGACCACAGCAACTTCTTGACCACGGTGGTGCAAAACAATGACTTTACCCCTACGGAAGCCAGCACCCAGACC
ATTAACTTTGATGAACGATCGCGGTGGGGCGGTCAGCTAAAGACCATCATGCATACTAACATGCCAAACGTGAACGA
GTATATGTTTAGTAACAAGTTCAAAGCGCGTGTGATGGTGTCCAGAAAACCTCCCGACGGTGCTGCAGTTGGGGATA
CTTATGATCACAAGCAGGATATTTTGAAATATGAGTGGTTCGAGTTTACTTTGCCAGAAGGCAACTTTTCAGTTACT
ATGACTATTGATTTGATGAACAATGCCATCATAGATAATTACTTGAAAGTGGGTAGACAGAATGGAGTGCTTGAAAG
TGACATTGGTGTTAAGTTCGACACCAGGAACTTCAAGCTGGGATGGGATCCCGAAACCAAGTTGATCATGCCTGGAG
TGTATACGTATGAAGCCTTCCATCCTGACATTGTCTTACTGCCTGGCTGCGGAGTGGATTTTACCGAGAGTCGTTTG
AGCAACCTTCTTGGTATCAGAAAAAAACAGCCATTTCAAGAGGGTTTTAAGATTTTGTATGAAGATTTAGAAGGTGG
TAATATTCCGGCCCTCTTGGATGTAGATGCCTATGAGAACAGTAAGAAAGAACAAAAAGCCAAAATAGAAGCTGCTA
CAGCTGCTGCAGAAGCTAAGGCAAACATAGTTGCCAGCGACTCTACAAGGGTTGCTAACGCTGGAGAGGTCAGAGGA
GACAATTTTGCGCCAACACCTGTTCCGACTGCAGAATCATTATTGGCCGATGTGTCTGAAGGAACGGACGTGAAACT
CACTATTCAACCTGTAGAAAAAGATAGTAAGAATAGAAGCTATAATGTGTTGGAAGACAAAATCAACACAGCCTATC
GCAGTTGGTATCTTTCGTACAATTATGGCGATCCCGAAAAAGGAGTGCGTTCCTGGACATTGCTCACCACCTCAGAT
GTCACCTGCGGAGCAGAGCAGGTCTACTGGTCGCTTCCAGACATGATGAAGGATCCTGTCACTTTCCGCTCCACTAG
ACAAGTCAGTAACTACCCTGTGGTGGGTGCAGAGCTTATGCCCGTCTTCTCAAAGAGCTTCTACAACGAACAAGCTG
TGTACTCCCAGCAGCTCCGCCAGTCCACCTCGCTTACGCACGTCTTCAACCGCTTTCCTGAGAACCAGATTTTAATC
CGTCCGCCGGCGCCCACCATTACCACCGTCAGTGAAAACGTTCCTGCTCTCACAGATCACGGGACCCTGCCGTTGCG
CAGCAGTATCCGGGGAGTCCAACGTGTGACCGTTACTGACGCCAGACGCCGCACCTGTCCCTACGTGTACAAGGCAC
TGGGCATAGTCGCACCGCGCGTCCTTTCAAGCCGCACTTTCTAAAAAAAAAAAAAATGTCCATTCTTATCTCGCCCA
GTAATAACACCGGTTGGGGTCTGCGCGCTCCAAGCAAGATGTACGGAGGCGCACGCAAACGTTCTACCCAACATCCT
GTCCGTGTTCGCGGACATTTTCGCGCTCCATGGGGCGCCCTCAAGGGCCGCACTCGCGTTCGAACCACCGTCGATGA
TGTAATCGATCAGGTGGTTGCCGACGCCCGTAATTATACTCCTACTGCGCCTACATCTACTGTGGATGCAGTTATTG
ACAGTGTAGTGGCTGACGCTCGCAACTATGCTCGACGTAAGAGCCGGCGAAGGCGCATTGCCAGACGCCACCGAGCT
ACCACTGCCATGCGAGCCGCAAGAGCTCTGCTACGAAGAGCTAGACGCGTGGGGCGAAGAGCCATGCTTAGGGCGGC
CAGACGTGCAGCTTCGGGCGCCAGCGCCGGCAGGTCCCGCAGGCAAGCAGCCGCTGTCGCAGCGGCGACTATTGCCG
ACATGGCCCAATCGCGAAGAGGCAATGTATACTGGGTGCGTGACGCTGCCACCGGTCAACGTGTACCCGTGCGCACC
CGTCCCCCTCGCACTTAGAAGATACTGAGCAGTCTCCGATGTTGTGTCCCAGCGGCGAGGATGTCCAAGCGCAAATA
CAAGGAAGAAATGCTGCAGGTTATCGCACCTGAAGTCTACGGCCAACCGTTGAAGGATGAAAAAAAACCCCGCAAAA
TCAAGCGGGTTAAAAAGGACAAAAAAGAAGAGGAAGATGGCGATGATGGGCTGGCGGAGTTTGTGCGCGAGTTTGCC
CCACGGCGACGCGTGCAATGGCGTGGGCGCAAAGTTCGACATGTGTTGAGACCTGGAACTTCGGTGGTCTTTACACC
CGGCGAGCGTTCAAGCGCTACTTTTAAGCGTTCCTATGATGAGGTGTACGGGGATGATGATATTCTTGAGCAGGCGG
CTGACCGATTAGGCGAGTTTGCTTATGGCAAGCGTAGTAGAATAACTTCCAAGGATGAGACAGTGTCGATACCCTTG
GATCATGGAAATCCCACCCCTAGTCTTAAACCGGTCACTTTGCAGCAAGTGTTACCCGTAACTCCGCGAACAGGTGT
TAAACGCGAAGGTGAAGATTTGTATCCCACTATGCAACTGATGGTACCCAAACGCCAGAAGTTGGAGGACGTTTTGG
AGAAAGTAAAAGTGGATCCAGATATTCAACCTGAGGTTAAAGTGAGACCCATTAAGCAGGTAGCGCCTGGTCTGGGG
GTACAAACTGTAGACATTAAGATTCCCACTGAAAGTATGGAAGTGCAAACTGAACCCGCAAAGCCTACTGCCACCTC
CACTGAAGTGCAAACGGATCCATGGATGCCCATGCCTATTACAACTGACGCCGCCGGTCCCACTCGAAGATCCCGAC
GAAAGTACGGTCCAGCAAGTCTGTTGATGCCCAATTATGTTGTACACCCATCTATTATTCCTACTCCTGGTTACCGA
GGCACTCGCTACTATCGCAGCCGAAACAGTACCTCCCGCCGTCGCCGCAAGACACCTGCAAATCGCAGTCGTCGCCG
TAGACGCACAAGCAAACCGACTCCCGGCGCCCTGGTGCGGCAAGTGTACCGCAATGGTAGTGCGGAACCTTTGACAC
TGCCGCGTGCGCGTTACCATCCGAGTATCATCACTTAATCAATGTTGCCGCTGCCTCCTTGCAGATATGGCCCTCAC
TTGTCGCCTTCGCGTTCCCATCACTGGTTACCGAGGAAGAAACTCGCGCCGTAGAAGAGGGATGTTGGGACGCGGAA
TGCGACGCTACAGGCGACGGCGTGCTATCCGCAAGCAATTGCGGGGTGGTTTTTTACCAGCCTTAATTCCAATTATC
GCTGCTGCAATTGGCGCGATACCAGGCATAGCTTCCGTGGCGGTTCAGGCCTCGCAACGACATTGACATTGGAAAAA
AACGTATAAATAAAAAAAAAAAAATACAATGGACTCTGACACTCCTGGTCCTGTGACTATGTTTTCTTAGAGATGGA
AGACATCAATTTTTCATCCTTGGCTCCGCGACACGGCACGAAGCCGTACATGGGCACCTGGAGCGACATCGGCACGA
GCCAACTGAACGGGGGCGCCTTCAATTGGAGCAGTATCTGGAGCGGGCTTAAAAATTTTGGCTCAACCATAAAAACA
TACGGGAACAAAGCTTGGAACAGCAGTACAGGACAGGCGCTTAGAAATAAACTTAAAGACCAGAACTTCCAACAAAA
AGTAGTCGATGGGATAGCTTCCGGCATCAATGGAGTGGTAGATTTGGCTAACCAGGCTGTGCAGAAAAAGATAAACA
GTCGTTTGGACCCGCCGCCAGCAACCCCAGGTGAAATGCAAGTGGAGGAAGAAATTCCTCCGCCAGAAAAACGAGGC
GACAAGCGTCCGCGTCCCGATTTGGAAGAGACGCTGGTGACGCGCGTAGATGAACCGCCTTCTTATGAGGAAGCAAC
GAAGCTTGGAATGCCCACCACTAGACCGATAGCCCCAATGGCCACCGGGGTGATGAAACCTTCTCAGTTGCATCGAC
CCGTCACCTTGGATTTGCCCCCTCCCCCTGCTGCTACTGCTGTACCCGCTTCTAAGCCTGTCGCTGCCCCGAAACCA
GTCGCCGTAGCCAGGTCACGTCCCGGGGGCGCTCCTCGTCCAAATGCGCACTGGCAAAATACTCTGAACAGCATCGT
GGGTCTAGGCGTGCAAAGTGTAAAACGCCGTCGCTGCTTTTAATTAAATATGGAGTAGCGCTTAACTTGCCTATCTG
TGTATATGTGTCATTACACGCCGTCACAGCAGCAGAGGAAAAAAGGAAGAGGTCGTGCGTCGACGCTGAGTTACTTT
CAAGATGGCCACCCCATCGATGCTGCCCCAATGGGCATACATGCACATCGCCGGACAGGATGCTTCGGAGTACCTGA
GTCCGGGTCTGGTGCAGTTCGCCCGCGCCACAGACACCTACTTCAATCTGGGAAATAAGTTTAGAAATCCCACCGTA
GCGCCGACCCACGATGTGACCACCGACCGTAGCCAGCGGCTCATGTTGCGCTTCGTGCCCGTTGACCGGGAGGACAA
TACATACTCTTACAAAGTGCGGTACACCCTGGCCGTGGGCGACAACAGAGTGCTGGATATGGCCAGCACGTTCTTTG
ACATTAGGGGTGTGTTGGACAGAGGTCCCAGTTTCAAACCCTATTCTGGTACGGCTTACAACTCCCTGGCTCCTAAA
GGCGCTCCAAATACATCTCAGTGGATTGCAGAAGGTGTAAAAAATACAACTGGTGAGGAACACGTAACAGAAGAGGA
AACCAATACTACTACTTACACTTTTGGCAATGCTCCTGTAAAAGCTGAAGCTGAAATTACAAAAGAAGGACTCCCAG
TAGGTTTGGAAGTTTCAGATGAAGAAAGTAAACCGATTTATGCTGATAAAACATATCAGCCAGAACCTCAGCTGGGA
GATGAAACTTGGACTGACCTTGATGGAAAAACCGAAAAGTATGGAGGCAGGGCTCTCAAACCCGATACTAAGATGAA
ACCATGCTACGGGTCCTTTGCCAAACCTACTAATGTGAAAGGCGGTCAGGCAAAACAAAAAACAACGGAGCAGCCAA
ATCAGAAAGTCGAATATGATATCGACATGGAGTTTTTTGATGCGGCATCGCAGAAAACAAACTTAAGTCCTAAAATT
GTCATGTATGCAGAAAATGTAAATTTGGAAACTCCAGACACTCATGTAGTGTACAAACCTGGAACAGAAGACACAAG
TTCCGAAGCTAATTTGGGACAACAATCTATGCCCAACAGACCCAACTACATTGGCTTCAGAGATAACTTTATTGGAC
TTATGTACTATAACAGTACTGGTAACATGGGGGTGCTGGCTGGTCAAGCGTCTCAGTTAAATGCAGTGGTTGACTTG
CAGGACAGAAACACAGAACTTTCTTACCAACTCTTGCTTGACTCTCTGGGCGACAGAACCAGATACTTTAGCATGTG
GAATCAGGCTGTGGACAGTTATGATCCTGATGTACGTGTTATTGAAAATCATGGTGTGGAAGATGAACTTCCCAACT
ACTGTTTTCCACTGGACGGCATAGGTGTTCCAACAACCAGTTACAAATCAATAGTTCCAAATGGAGACAATGCGCCT
AATTGGAAGGAACCTGAAGTAAATGGAACAAGTGAGATCGGACAGGGTAATTTGTTTGCCATGGAAATTAACCTTCA
AGCCAATCTATGGCGAAGTTTCCTTTATTCCAATGTGGCTCTATATCTCCCAGACTCGTACAAATACACCCCGTCCA
ATGTCACTCTTCCAGAAAACAAAAACACCTACGACTACATGAACGGGCGGGTGGTGCCGCCATCTCTAGTAGACACC
TATGTGAACATTGGTGCCAGGTGGTCTCTGGATGCCATGGACAATGTCAACCCATTCAACCACCACCGTAACGCTGG
CTTGCGTTACCGATCCATGCTTCTGGGTAACGGACGTTATGTGCCTTTCCACATACAAGTGCCTCAAAAATTCTTCG
CTGTTAAAAACCTGCTGCTTCTCCCAGGCTCCTACACTTATGAGTGGAACTTTAGGAAGGATGTGAACATGGTTCTA
CAGAGTTCCCTCGGTAACGACCTGCGGGTAGATGGCGCCAGCATCAGTTTCACGAGCATCAACCTCTATGCTACTTT
TTTCCCCATGGCTCACAACACCGCTTCCACCCTTGAAGCCATGCTGCGGAATGACACCAATGATCAGTCATTCAACG
ACTACCTATCTGCAGCTAACATGCTCTACCCCATTCCTGCCAATGCAACCAATATTCCCATTTCCATTCCTTCTCGC
AACTGGGCGGCTTTCAGAGGCTGGTCATTTACCAGACTGAAAACCAAAGAAACTCCCTCTTTGGGGTCTGGATTTGA
CCCCTACTTTGTCTATTCTGGTTCTATTCCCTACCTGGATGGTACCTTCTACCTGAACCACACTTTTAAGAAGGTTT
CCATCATGTTTGACTCTTCAGTGAGCTGGCCTGGAAATGACAGGTTACTATCTCCTAACGAATTTGAAATAAAGCGC
ACTGTGGATGGCGAAGGCTACAACGTAGCCCAATGCAACATGACCAAAGACTGGTTCTTGGTACAGATGCTCGCCAA
CTACAACATCGGCTATCAGGGCTTCTACATTCCAGAAGGATACAAAGATCGCATGTATTCATTTTTCAGAAACTTCC
AGCCCATGAGCAGGCAGGTGGTTGATGAGGTCAATTACAAAGACTTCAAGGCCGTCGCCATACCCTACCAACACAAC
AACTCTGGCTTTGTGGGTTACATGGCTCCGACCATGCGCCAAGGTCAACCCTATCCCGCTAACTATCCCTATCCACT
CATTGGAACAACTGCCGTAAATAGTGTTACGCAGAAAAAGTTCTTGTGTGACAGAACCATGTGGCGCATACCGTTCT
CGAGCAACTTCATGTCTATGGGGGCCCTTACAGACTTGGGACAGAATATGCTCTATGCCAACTCAGCTCATGCTCTG
GACATGACCTTTGAGGTGGATCCCATGGATGAGCCCACCCTGCTTTATCTTCTCTTCGAAGTTTTCGACGTGGTCAG
AGTGCATCAGCCACACCGCGGCATCATCGAGGCAGTCTACCTGCGTACACCGTTCTCGGCCGGTAACGCTACCACGT
AAGAAGCTTCTTGCTTCTTGCAAATAGCAGCTGCAACCATGGCCTGCGGATCCCAAAACGGCTCCAGCGAGCAAGAG
CTCAGAGCCATTGTCCAAGACCTGGGTTGCGGACCCTATTTTTTGGGAACCTACGATAAGCGCTTCCCGGGGTTCAT
GGCCCCCGATAAGCTCGCCTGTGCCATTGTAAATACGGCCGGACGTGAGACGGGGGGAGAGCACTGGTTGGCTTTCG
GTTGGAACCCACGTTCTAACACCTGCTACCTTTTTGATCCTTTTGGATTCTCGGATGATCGTCTCAAACAGATTTAC
CAGTTTGAATATGAGGGTCTCCTGCGCCGCAGCGCTCTTGCTACCAAGGACCGCTGTATTACGCTGGAAAAATCTAC
CCAGACCGTGCAGGGTCCCCGTTCTGCCGCCTGCGGACTTTTCTGCTGCATGTTCCTTCACGCCTTTGTGCACTGGC
CTGACCGTCCCATGGACGGAAACCCCACCATGAAATTGCTAACTGGAGTGCCAAACAACATGCTTCATTCTCCTAAA
GTCCAGCCCACCCTGTGTGACAATCAAAAAGCACTCTACCATTTTCTTAATACCCATTCGCCTTATTTTCGCTCCCA
TCGTACACACATCGAAAGGGCCACTGCGTTCGACCGTATGGATGTTCAATAATGACTCATGTAAACAACGTGTTCAA
TAAACATCACTTTATTTTTTTACATGTATCAAGGCTCTGCATTACTTATTTATTTACAAGTCGAATGGGTTCTGACG
AGAATCAGAATGACCCGCAGGCAGTGATACGTTGCGGAACTGATACTTGGGTTGCCACTTGAATTCGGGAATCACCA
ACTTGGGAACCGGTATATCGGGCAGGATGTCACTCCACAGCTTTCTGGTCAGCTGCAAAGCTCCAAGCAGGTCAGGA
GCCGAAATCTTGAAATCACAATTAGGACCAGTGCTTTGAGCGCGAGAGTTGCGGTACACCGGATTGCAGCACTGAAA
CACCATCAGCGACGGATGTCTCACGCTTGCCAGCACGGTGGGATCTGCAATCATGCCCACATCCAGATCTTCAGCAT
TGGCAATGCTGAACGGGGTCATCTTGCAGGTCTGCCTACCCATGGCGGGCACCCAATTAGGCTTGTGGTTGCAATCG
CAGTGCAGGGGGATCAGTATCATCTTGGCCTGATCCTGTCTGATTCCTGGATACACGGCTCTCATGAAAGCATCATA
TTGCTTGAAAGCCTGCTGGGCTTTACTACCCTCGGTATAAAACATCCCGCAGGACCTGCTCGAAAACTGGTTAGCTG
CACAGCCGGCATCATTCACACAGCAGCGGGCGTCATTGTTAGCTATTTGCACCACACTTCTGCCCCAGCGGTTTTGG
GTGATTTTGGTTCGCTCGGGATTCTCCTTTAAGGCTCGTTGTCCGTTCTCGCTGGCCACATCCATCTCGATAATCTG
CTCCTTCTGAATCATAATATTGCCATGCAGGCACTTCAGCTTGCCCTCATAATCATTGCAGCCATGAGGCCACAACG
CACAGCCTGTACATTCCCAATTATGGTGGGCGATCTGAGAAAAAGAATGTATCATTCCCTGCAGAAATCTTCCCATC
ATCGTGCTCAGTGTCTTGTGACTAGTGAAAGTTAACTGGATGCCTCGGTGCTCCTCGTTTACGTACTGGTGACAGAT
GCGCTTGTATTGTTCGTGTTGCTCAGGCATTAGTTTAAAAGAGGTTCTAAGTTCGTTATCCAGCCTGTACTTCTCCA
TCAGCAGACACATCACTTCCATGCCTTTCTCCCAAGCAGACACCAGGGGCAAGCTAATCGGATTCTTAACAGTGCAG
GCAGCAGCTCCTTTAGCCAGAGGGTCATCTTTAGCGATCTTCTCAATGCTTCTTTTGCCATCCTTCTCAACGATGCG
CACGGGCGGGTAGCTGAAACCCACTGCTACAAGTTGCGCCTCTTCTCTTTCTTCTTCGCTGTCTTGACTGATGTCTT
GCATGGGGATATGTTTGGTCTTCCTTGGCTTCTTTTTGGGGGGTATCGGAGGAGGAGGACTGTCGCTCCGTTCCGGA
GACAGGGAGGATTGTGACGTTTCGCTCACCATTACCAACTGACTGTCGGTAGAAGAACCTGACCCCACACGGCGACA
GGTGTTTCTCTTCGGGGGCAGAGGTGGAGGCGATTGCGAAGGGCTGCGGTCCGACCTGGAAGGCGGATGACTGGCAG
AACCCCTTCCGCGTTCGGGGGTGTGCTCCCTGTGGCGGTCGCTTAACTGATTTCCTTCGCGGCTGGCCATTGTGTTC
TCCTAGGCAGAGAAACAACAGACATGGAAACTCAGCCATTGCTGTCAACATCGCCACGAGTGCCATCACATCTCGTC
CTCAGCGACGAGGAAAAGGAGCAGAGCTTAAGCATTCCACCGCCCAGTCCTGCCACCACCTCTACCCTAGAAGATAA
GGAGGTCGACGCATCTCATGACATGCAGAATAAAAAAGCGAAAGAGTCTGAGACAGACATCGAGCAAGACCCGGGCT
ATGTGACACCGGTGGAACACGAGGAAGAGTTGAAACGCTTTCTAGAGAGAGAGGATGAAAACTGCCCAAAACAACGA
GCAGATAACTATCACCAAGATGCTGGAAATAGGGATCAGAACACCGACTACCTCATAGGGCTTGACGGGGAAGACGC
GCTCCTTAAACATCTAGCAAGACAGTCGCTCATAGTCAAGGATGCATTATTGGACAGAACTGAAGTGCCCATCAGTG
TGGAAGAGCTCAGCCGCGCCTACGAGCTTAACCTCTTTTCACCTCGTACTCCCCCCAAACGTCAGCCAAACGGCACC
TGCGAGCCAAATCCTCGCTTAAACTTTTATCCAGCTTTTGCTGTGCCAGAAGTACTGGCTACCTATCACATCTTTTT
TAAAAATCAAAAAATTCCAGTCTCCTGCCGCGCTAATCGCACCCGCGCCGATGCCCTACTCAATCTGGGACCTGGTT
CACGCTTACCTGATATAGCTTCCTTGGAAGAGGTTCCAAAGATCTTCGAGGGTCTGGGCAATAATGAGACTCGGGCC
GCAAATGCTCTGCAAAAGGGAGAAAATGGCATGGATGAGCATCACAGCGTTCTGGTGGAATTGGAAGGCGATAATGC
CAGACTCGCAGTACTCAAGCGAAGCATCGAGGTCACACACTTCGCATATCCCGCTGTCAACCTGCCCCCTAAAGTCA
TGACGGCGGTCATGGACCAGTTACTCATTAAGCGCGCAAGTCCCCTTTCAGAAGACATGCATGACCCAGATGCCTGT
GATGAGGGTAAACCAGTGGTCAGTGATGAGCAGCTAACCCGATGGCTGGGCACCGACTCTCCCAGGGATTTGGAAGA
GCGTCGCAAGCTTATGATGGCCGTGGTGCTGGTTACCGTAGAACTAGAGTGTCTCCGACGTTTCTTTACCGATTCAG
AAACCTTGCGCAAACTCGAAGAGAATCTGCACTACACTTTTAGACACGGCTTTGTGCGGCAGGCATGCAAGATATCT
AACGTGGAACTCACCAACCTGGTTTCCTACATGGGTATTCTGCATGAGAATCGCCTAGGACAAAGCGTGCTGCACAG
CACCCTGAAGGGGGAAGCCCGCCGTGATTACATCCGCGATTGTGTCTATCTGTACCTGTGCCACACGTGGCAAACCG
GCATGGGTGTATGGCAGCAATGTTTAGAAGAACAGAACTTGAAAGAGCTTGACAAGCTCTTACAGAAATCTCTTAAG
GTTCTGTGGACAGGGTTCGACGAGCGCACCGTCGCTTCCGACCTGGCAGACCTCATCTTCCCAGAGCGTCTCAGGGT
TACTTTGCGAAACGGATTGCCTGACTTTATGAGCCAGAGCATGCTTAACAATTTTCGCTCTTTCATCCTGGAACGCT
CCGGTATCCTGCCCGCCACCTGCTGCGCACTGCCCTCCGACTTTGTGCCTCTCACCTACCGCGAGTGCCCCCCGCCG
CTATGGAGTCACTGCTACCTGTTCCGTCTGGCCAACTATCTCTCCTACCACTCGGATGTGATCGAGGATGTGAGCGG
AGACGGCTTGCTGGAGTGTCACTGCCGCTGCAATCTGTGCACGCCCCACCGGTCCCTAGCTTGCAACCCCCAGTTGA
TGAGCGAAACCCAGATAATAGGCACCTTTGAATTGCAAGGCCCCAGCAGCCAAGGCGATGGGTCTTCTCCTGGGCAA
AGTTTAAAACTGACCCCGGGACTGTGGACCTCCGCCTACTTGCGCAAGTTTGCTCCGGAAGATTACCACCCCTATGA
AATCAAGTTCTATGAGGACCAATCACAGCCTCCAAAGGCCGAACTTTCGGCCTGCGTCATCACCCAGGGGGCAATTC
TGGCCCAATTGCAAGCCATCCAAAAATCCCGCCAAGAATTTCTACTGAAAAAGGGTAAGGGGGTCTACCTTGACCCC
CAGACCGGCGAGGAACTCAACACAAGGTTCCCTCAGGATGTCCCAACGACGAGAAAACAAGAAGTTGAAGGTGCAGC
CGCCGCCCCCAGAAGATATGGAGGAAGATTGGGACAGTCAGGCAGAGGAGGCGGAGGAGGACAGTCTGGAGGACAGT
CTGGAGGAAGACAGTTTGGAGGAGGAAAACGAGGAGGCAGAGGAGGTGGAAGAAGTAACCGCCGACAAACAGTTATC
CTCGGCTGCGGAGACAAGCAACAGCGCTACCATCTCCGCTCCGAGTCGAGGAACCCGGCGGCGTCCCAGCAGTAGAT
GGGACGAGACCGGACGCTTCCCGAACCCAACCAGCGCTTCCAAGACCGGTAAGAAGGATCGGCAGGGATACAAGTCC
TGGCGGGGGCATAAGAATGCCATCATCTCCTGCTTGCATGAGTGCGGGGGCAACATATCCTTCACGCGGCGCTACTT
GCTATTCCACCATGGGGTGAACTTTCCGCGCAATGTTTTGCATTACTACCGTCACCTCCACAGCCCCTACTATAGCC
AGCAAATCCCGGCAGTCTCGACAGATAAAGACAGCGGCGGCGACCTCCAACAGAAAACCAGCAGCGGCAGTTAGAAA
ATACACAACAAGTGCAGCAACAGGAGGATTAAAGATTACAGCCAACGAGCCAGCGCAAACCCGAGAGTTAAGAAATC
GGATCTTTCCAACCCTGTATGCCATCTTCCAGCAGAGTCGGGGTCAAGAGCAGGAACTGAAAATAAAAAACCGATCT
CTGCGTTCGCTCACCAGAAGTTGTTTGTATCACAAGAGCGAAGATCAACTTCAGCGCACTCTCGAGGACGCCGAGGC
TCTCTTCAACAAGTACTGCGCGCTGACTCTTAAAGAGTAGGCAGCGACCGCGCTTATTCAAAAAAGGCGGGAATTAC
ATCATCCTCGACATGAGTAAAGAAATTCCCACGCCTTACATGTGGAGTTATCAACCCCAAATGGGATTGGCGGCAGG
CGCCTCCCAGGACTACTCCACCCGCATGAATTGGCTCAGCGCCGGGCCTTCTATGATTTCTCGAGTTAATGATATAC
GCGCCTACCGAAACCAAATACTTTTGGAACAGTCAGCTCTTACCACCACGCCCCGCCAACACCTTAATCCCAGAAAT
TGGCCCGCCGCCCTAGTGTACCAGGAAAGTCCCGCTCCCACCACTGTATTACTTCCTCGAGACGCCCAGGCCGAAGT
CCAAATGACTAATGCAGGTGCGCAGTTAGCTGGCGGCTCCACCCTATGTCGTCACAGGCCTCGGCATAATATAAAAC
GCCTGATGATCAGAGGCCGAGGTATCCAGCTCAACGACGAGTCGGTGAGCTCTCCGCTTGGTCTACGACCAGACGGA
ATCTTTCAGATTGCCGGCTGCGGGAGATCTTCCTTCACCCCTCGTCAGGCTGTTCTGACTTTGGAAAGTTCGTCTTC
GCAACCCCGCTCGGGCGGAATCGGGACCGTTCAATTTGTGGAGGAGTTTACTCCCTCTGTCTACTTCAACCCCTTCT
CCGGATCTCCTGGGCATTACCCGGACGAGTTCATACCGAACTTCGACGCGATTAGCGAGTCAGTGGACGGCTACGAT
TGATGTCTGGTGACGCGGCTGAGCTATCTCGGCTGCGACATCTAGACCACTGCCGCCGCTTTCGCTGCTTTGCCCGG
GAACTCATTGAGTTCATCTACTTCGAACTCCCCAAGGATCACCCTCAAGGTCCGGCCCACGGAGTGCGGATTTCTAT
CGAAGGCAAAATAGACTCTCGCCTGCAACGAATTTTCTCCCAGCGGCCCGTGCTGATCGAGCGAGACCAGGGAAACA
CCACGGTTTCCATCTACTGCATTTGTAATCACCCCGGATTGCATGAAAGCCTTTGCTGTCTTATGTGTACTGAGTTT
AATAAAAACTGAATTAAGACTCTCCTACGGACTGCCGCTTCTTCAACCCGGATTTTACAACCAGAAGAACGAAACTT
TTCCTGTCGTCCAGGACTCTGTTAACTTCACCTTTCCTACTCACAAACTAGAAGCTCAACGACTACACCGCTTTTCC
AGAAGCATTTTCCCTACTAATACTACTTTCAAAACCGGAGGTGAGCTCCAAGGTCTTCCTACAGAAAACCCTTGGGT
GGAAGCGGGCCTTGTAGTGCTAGGAATTCTTGCGGGTGGGCTTGTGATTATTCTTTGCTACCTATACACACCTTGCT
TCACTTTCTTAGTGGTGTTGTGGTATTGGTTTAAAAAATGGGGCCCATACTAGTCTTGCTTGTTTTACTTTCGCTTT
TGGAACCGGGTTCTGCCAATTACGATCCATGTCTAGACTTCGACCCAGAAAACTGCACACTTACTTTTGCACCCGAC
ACAAGCCGCATCTGTGGAGTTCTTATTAAGTGCGGATGGGAATGCAGGTCCGTTGAAATTACACACAATAACAAAAC
CTGGAACAATACCTTATCCACCACATGGGAGCCAGGAGTTCCCGAGTGGTACACTGTCTCTGTCCGAGGTCCTGACG
GTTCCATCCGCATTAGTAACAACACTTTCATTTTTTCTGAAATGTGCGATCTGGCCATGTTCATGAGCAAACAGTAT
TCTCTATGGCCTCCTAGCAAGGACAACATCGTAACGTTCTCCATTGCTTATTGCTTGTGCGCTTGCCTTCTTACTGC
TTTACTGTGCGTATGCATACACCTGCTTGTAACCACTCGCATCAAAAACGCCAATAACAAAGAAAAAATGCCTTAAC
CTCTTTCTGTTTACAGACATGGCTTCTCTTACATCTCTCATATTTGTCAGCATTGTCACTGCCGCTCATGGACAAAC
AGTCGTCTCTATCCCTCTAGGACATAATTACACTCTCATAGGACCCCCAATCACTTCAGAGGTCATCTGGACCAAAC
TGGGAAGCGTTGATTACTTTGATATAATCTGCAACAAAACAAAACCAATAATAGTAACTTGCAACATACAAAATCTT
ACATTGATTAATGTTAGCAAAGTTTACAGCGGTTACTATTATGGTTATGACAGATACAGTAGTCAATATAGAAATTA
CTTGGTTCGTGTTACCCAGTTGAAAACCACGAAAATGCCAAATATGGCAAAGATTCGATCCGATGACAATTCTCTAG
AAACTTTTACATCTCCCACCACACCCGACGAAAAAAACATCCCAGATTCAATGATTGCAATTGTTGCAGCGGTGGCA
GTGGTGATGGCACTAATAATAATATGCATGCTTTTATATGCTTGTCGCTACAAAAAGTTTCATCCTAAAAAACAAGA
TCTCCTACTAAGGCTTAACATTTAATTTCTTTTTATACAGCCATGGTTTCCACTACCACATTCCTTATGCTTACTAG
TCTCGCAACTCTGACTTCTGCTCGCTCACACCTCACTGTAACTATAGGCTCAAACTGCACACTAAAAGGACCTCAAG
GTGGTCATGTCTTTTGGTGGAGAATATATGACAATGGATGGTTTACAAAACCATGTGACCAACCTGGTAGATTTTTC
TGCAACGGCAGAGACCTAACCATTATCAACGTGACAGCAAATGACAAAGGCTTCTATTATGGAACCGACTATAAAAG
TAGTTTAGATTATAACATTATTGTACTGCCATCTACCACTCCAGCACCCCGCACAACTACTTTCTCTAGCAGCAGTG
TCGCTAACAATACAATTTCCAATCCAACCTTTGCCGCGCTTTTAAAACGCACTGTGAATAATTCTACAACTTCACAT
ACAACAATTTCCACTTCAACAATCAGCATTATCGCTGCAGTGACAATTGGAATATCTATTCTTGTTTTTACCATAAC
CTACTACGCCTGCTGCTATAGAAAAGACAAACATAAAGGTGATCCATTACTTAGATTTGATATTTAATTTGTTCTTT
TTTTTTTTATTTACAGTATGGTGAACACCAATCATGGTACCTAGAAATTTCTTCTTCACCATACTCATTTGTGCATT
TAATGTTTGCGCTACTTTCACAGCAGTAGCCACAGCAACCCCAGACTGTATAGGAGCATTTGCTTCCTATGCACTTT
TTGCTTTTGTTACTTGCATCTGCGTATGTAGCATAGTCTGCCTGGTTATTAATTTTTTCCAACTTATAGACTGGATC
CTTGTGCGAATTGCCTACCTGCGCCACCATCCCGAATACCGCAACCAAAATATCGCGGCACTTCTTAGACTCATCTA
AAACCATGCAGGCTATACTACCAATATTTTTGCTTCTATTGCTTCCCTACGCTGTCTCAACCCCAGCTGCCTATAGT
ACTCCACCAGAACACCTTAGAAAATGCAAATTCCAACAACCGTGGTCATTTCTTGCTTGCTATCGAGAAAAATCAGA
AATTCCCCCAAATTTAATAATGATTGCTGGAATAATTAATATAATCTGTTGCACCATAATTTCATTTTTGATATACC
CCCTATTTGATTTTGGCTGGAATGCTCCCAATGCACATGATCATCCACAAGACCCAGAGGAACACATTCCCCTACAA
AACATGCAACATCCAATAGCGCTAATAGATTACGAAAGTGAACCACAACCCCCACTACTCCCTGCTATTAGTTACTT
CAACCTAACCGGCGGAGATGACTGAAACACTCACCACCTCCAATTCCGCCGAGGATCTGCTCGATATGGACGGCCGC
GTCTCAGAACAGCGACTCGCCCAACTACGCATCCGCCAGCAGCAGGAACGCGCGGCCAAAGAGCTCAGAGATGTCAT
CCAAATTCACCAATGCAAAAAAGGCATATTCTGTTTGGTAAAACAAGCCAAGATATCCTACGAGATCACCGCTACTG
ACCATCGCCTCTCTTACGAACTTGGCCCCCAACGACAAAAATTTACCTGCATGGTGGGAATCAACCCCATAGTTATC
ACCCAGCAAAGTGGAGATACTAAGGGTTGCATTCACTGCTCCTGCGATTCCATCGAGTGCACCTACACCCTGCTGAA
GACCCTATACGGCCTAAGAGACCTGCTACCAATGAATTAAAAAATGATTAATAAAAAATCACTTACTTGAAATCAGC
AATAAGGTCTCTGTTGAAATTTTCTCCCAGCAGCACCTCACTTCCCTCTTCCCAACTCTGGTATTCTAAACCCCGTT
CAGCGGCATACTTTCTCCATACTTTAAAGGGGATGTCAAATTTTAGCTCCTCTCCTGTACCCACAATCTTCATGTCT
TTCTTCCCAGATGACCAAGAGAGTCCGGCTCAGTGACTCCTTCAACCCTGTCTACCCCTATGAAGATGAAAGCACCT
CCCAACACCCCTTTATAAACCCAGGGTTTATTTCCCCAAATGGCTTCACACAAAGCCCAAACGGAGTTCTTACTTTA
AAATGTTTAACCCCACTAACAACCACAGGCGGATCTCTACAGCTAAAAGTGGGAGGGGGACTTACAGTGGATGACAC
CAACGGTTTTTTGAAAGAAAACATAAGTGCCACCACACCACTCGTTAAGACTGGTCACTCTATAGGTTTACCACTAG
GAGCCGGATTGGGAACGAATGAAAATAAACTTTGTATCAAATTAGGACAAGGACTTACATTCAATTCAAACAACATT
TGCATTGATGACAATATTAACACCTTATGGACAGGAGTCAACCCCACCGAAGCCAACTGTCAAATCATGAACTCCAG
TGAATCTAATGATTGCAAATTAATTCTAACACTAGTTAAAACTGGAGCACTAGTCACTGCATTTGTTTATGTTATAG
GAGTATCTAACAATTTTAATATGCTAACTACACACAGAAATATAAATTTTACTGCAGAGCTGTTTTTCGATTCTACT
GGTAATTTACTAACTAGACTCTCATCCCTCAAAACTCCACTTAATCATAAATCAGGACAAAACATGGCTACTGGTGC
CATTACTAATGCTAAAGGTTTCATGCCCAGCACGACTGCCTATCCTTTCAATGATAATTCTAGAGAAAAAGAAAACT
ACATTTACGGAACTTGTTACTACACAGCTAGTGATCGCACTGCTTTTCCCATTGACATATCTGTCATGCTTAACCGA
AGAGCAATAAATGACGAGACATCATATTGTATTCGTATAACTTGGTCCTGGAACACAGGAGATGCCCCAGAGGTGCA
AACCTCTGCTACAACCCTAGTCACCTCCCCATTTACCTTTTACTACATCAGAGAAGACGACTGACAAATAAAGTTTA
ACTTGTTTATTTGAAAATCAATTCACAAAATCCGAGTAGTTATTTTGCCTCCCCCTTCCCATTTAACAGAATACACC
AATCTCTCCCCACGCACAGCTTTAAACATTTGGATACCATTAGATATAGACATGGTTTTAGATTCCACATTCCAAAC
AGTTTCAGAGCGAGCCAATCTGGGGTCAGTGATAGATAAAAATCCATCGGGATAGTCTTTTAAAGCGCTTTCACAGT
CCAACTGCTGCGGATGCGACTCCGGAGTCTGGATCACGGTCATCTGGAAGAAGAACGATGGGAATCATAATCCGAAA
ACGGTATCGGACGATTGTGTCTCATCAAACCCACAAGCAGCCGCTGTCTGCGTCGCTCCGTGCGACTGCTGTTTATG
GGATCAGGGTCCACAGTGTCCTGAAGCATGATTTTAATAGCCCTTAACATCAACTTTCTGGTGCGATGCGCGCAGCA
ACGCATTCTGATTTCACTCAAATCTTTGCAGTAGGTACAACACATTATTACAATATTGTTTAATAAACCATAATTAA
AAGCGCTCCAGCCAAAACTCATATCTGATATAATCGCCCCTGCATGACCATCATACCAAAGTTTAATATAAATTAAA
TGACGTTCCCTCAAAAACACACTACCCACATACATGATCTCTTTTGGCATGTGCATATTAACAATCTGTCTGTACCA
TGGACAACGTTGGTTAATCATGCAACCCAATATAACCTTCCGGAACCACACTGCCAACACCGCTCCCCCAGCCATGC
ATTGAAGTGAACCCTGCTGATTACAATGACAATGAAGAACCCAATTCTCTCGACCGTGAATCACTTGAGAATGAAAA
ATATCTATAGTGGCACAACATAGACATAAATGCATGCATCTTCTCATAATTTTTAACTCCTCAGGATTTAGAAACAT
ATCCCAGGGAATAGGAAGCTCTTGCAGAACAGTAAAGCTGGCAGAACAAGGAAGACCACGAACACAACTTACACTAT
GCATAGTCATAGTATCACAATCTGGCAACAGCGGGTGGTCTTCAGTCATAGAAGCTCGGGTTTCATTTTCCTCACAA
CGTGGTAACTGGGCTCTGGTGTAAGGGTGATGTCTGGCGCATGATGTCGAGCGTGCGCGCAACCTTGTCATAATGGA
GTTGCTTCCTGACATTCTCGTATTTTGTATAGCAAAACGCGGCCCTGGCAGAACACACTCTTCTTCGCCTTCTATCC
TGCCGCTTAGCGTGTTCCGTGTGATAGTTCAAGTACAACCACACTCTTAAGTTGGTCAAAAGAATGCTGGCTTCAGT
TGTAATCAAAACTCCATCGCATCTAATCGTTCTGAGGAAATCATCCACGGTAGCATATGCAAATCCCAACCAAGCAA
TGCAACTGGATTGTGTTTCAAGCAGGAGAGGAGAGGGAAGAGACGGAAGAACCATGTTAATTTTTATTCCAAACGAT
CTCGCAGTACTTCAAATTGTAGATCGCGCAGATGGCATCTCTCGCCCCCACTGTGTTGGTGAAAAAGCACAGCTAGA
TCAAAAGAAATGCGATTTTCAAGGTGCTCAACGGTGGCTTCCAGCAAAGCCTCCACGCGCACATCCAAGAACAAAAG
AATACCAAAAGAAGGAGCATTTTCTAACTCCTCAATCATCATATTACATTCCTGCACCATTCCCAGATAATTTTCAG
CTTTCCAGCCTTGAATTATTCGTGTCAGTTCTTGTGGTAAATCCAATCCACACATTACAAACAGGTCCCGGAGGGCG
CCCTCCACCACCATTCTTAAACACACCCTCATAATGACAAAATATCTTGCTCCTGTGTCACCTGTAGCGAATTGAGA
ATGGCAACATCAATTGACATGCCCTTGGCTCTAAGTTCTTCTTTAAGTTCTAGTTGTAAAAACTCTCTCATATTATC
ACCAAACTGCTTAGCCAGAAGCCCCCCGGGAACAAGAGCAGGGGACGCTACAGTGCAGTACAAGCGCAGACCTCCCC
AATTGGCTCCAGCAAAAACAAGATTGGAATAAGCATATTGGGAACCGCCAGTAATATCATCGAAGTTGCTGGAAATA
TAATAAGGCAGAGTTTCTTGTAAAAATTGAATAAAAGAAAAATTTGCCAAAAAAACATTCAAAACCTCTGGGATGCA
AATGCAATAGGTTACCGCGCTGCGCTCCAACATTGTTAGTTTTGAATTAGTCTGCAAAAATAAAAAAAAAAACAAGC
GTCATATCATAGTAGCCTGACGAACAGATGGATAAATCAGTCTTTCCATCACAAGACAAGCCACAGGGTCTCCAGCT
CGACCCTCGTAAAACCTGTCATCATGATTAAACAACAGCACCGAAAGTTCCTCGCGGTGACCAGCATGAATAATTCT
TGATGAAGCATACAATCCAGACATGTTAGCATCAGTTAACGAGAAAAAACAGCCAACATAGCCTTTGGGTATAATTA
TGCTTAATCGTAAGTATAGCAAAGCCACCCCTCGCGGATACAAAGTAAAAGGCACAGGAGAATAAAAAATATAATTA
TTTCTCTGCTGCTGTTCAGGCAACGTCGCCCCCGGTCCCTCTAAATACACATACAAAGCCTCATCAGCCATGGCTTA
CCAGACAAAGTACAGCGGGCACACAAAGCACAAGCTCTAAAGTGACTCTCCAACCTCTCCACAATATATATATACAC
AAGCCCTAAACTGACGTAATGGGAGTAAAGTGTAAAAAATCCCGCCAAACCCAACACACACCCCGAAACTGCGTCAC
CAGGGAAAAGTACAGTTTCACTTCCGCAATCCCAACAGGCGTAACTTCCTCTTTCTCACGGTACGTGATATCCCACT
AACTTGCAACGTCATTTTCCCACGGTCGCACCGCCCCTTTTAGCCGTTAACCCCACAGCCAATCACCACACGATCCA
CACTTTTTAAAATCACCTCATTTACATATTGGCACCATTCCATCTATAAGGTATATTATTGATGATG

GenBank Accession No. YP_002213828 (SEQ ID NO: 220)

MTKRVRLSDSFNPVYPYEDESTSQHPFINPGFISPNGFTQSPNGVLTLKC
LTPLTTTGGSLQLKVGGGLTVDDINGELKENISATTPLVKTGHSIGLPLG
AGLGTNENKLCIKLGQGLTENSNNICIDDNINTLWTGVNPTEANCQIMNS
SESNDCKLILTLVKTGALVTAFVYVIGVSNNENMLTTHRNINFTAELFFD
STGNLLTRLSSLKTPLNHKSGQNMATGAITNAKGFMPSTTAYPFNDNSRE
KENYIYGTCYYTASDRTAFPIDISVMLNRRAINDETSYCIRITWSWNTGD
APEVQTSATTLVTSPFTFYYIREDD

GenBank Accession No. YP_002213807 (SEQ ID NO: 221)

MRRVVLGGAVVYPEGPPPSYESVMQQQQATAVMQSPLEAPFVPPRYLAPT
EGRNSIRYSELAPQYDTTRLYLVDNKSADIASLNYQNDHSNFLTTVVQNN
DFTPTEASTQTINFDERSRWGGQLKTIMHTNMPNVNEYMFSNKFKARVMV
SRKPPDGAAVGDTYDHKQDILKYEWFEFTLPEGNFSVTMTIDLMNNAIID
NYLKVGRQNGVLESDIGVKEDTRNFKLGWDPETKLIMPGVYTYEAFHPDI
VLLPGCGVDFTESRLSNLLGIRKKQPFQEGFKILYEDLEGGNIPALLDVD
AYENSKKEQKAKIEAATAAAEAKANIVASDSTRVANAGEVRGDNFAPTPV
PTAESLLADVSEGTDVKLTIQPVEKDSKNRSYNVLEDKINTAYRSWYLSY
NYGDPEKGVRSWTLLTTSDVTCGAEQVYWSLPDMMKDPVTFRSTRQVSNY
PVVGAELMPVFSKSFYNEQAVYSQQLRQSTSLTHVENRFPENQILIRPPA
PTITTVSENVPALTDHGTLPLRSSIRGVQRVTVTDARRRTCPYVYKALGI
VAPRVLSSRTF

GenBank Accession No. YP_002213812 (SEQ ID NO: 222)

MATPSMLPQWAYMHIAGQDASEYLSPGLVQFARATDTYFNLGNKFRNPTV
APTHDVTTDRSQRLMLRFVPVDREDNTYSYKVRYTLAVGDNRVLDMASTF
FDIRGVLDRGPSFKPYSGTAYNSLAPKGAPNTSQWIAEGVKNTTGEEHVT
EEETNTTTYTFGNAPVKAEAEITKEGLPVGLEVSDEESKPIYADKTYQPE
PQLGDETWTDLDGKTEKYGGRALKPDTKMKPCYGSFAKPTNVKGGQAKQK
TTEQPNQKVEYDIDMEFFDAASQKINLSPKIVMYAENVNLETPDTHVVYK
PGTEDTSSEANLGQQSMPNRPNYIGFRDNFIGLMYYNSTGNMGVLAGQAS
QLNAVVDLQDRNTELSYQLLLDSLGDRTRYFSMWNQAVDSYDPDVRVIEN
HGVEDELPNYCFPLDGIGVPTTSYKSIVPNGDNAPNWKEPEVNGTSEIGQ
GNLFAMEINLQANLWRSFLYSNVALYLPDSYKYTPSNVILPENKNTYDYM
NGRVVPPSLVDTYVNIGARWSLDAMDNVNPENHHRNAGLRYRSMLLGNGR
YVPFHIQVPQKFFAVKNLLLLPGSYTYEWNFRKDVNMVLQSSLGNDLRVD
GASISFTSINLYATFFPMAHNTASTLEAMLRNDTNDQSENDYLSAANMLY
PIPANATNIPISIPSRNWAAFRGWSFTRLKTKETPSLGSGFDPYFVYSGS
IPYLDGTFYLNHTFKKVSIMFDSSVSWPGNDRLLSPNEFEIKRTVDGEGY
NVAQCNMTKDWFLVQMLANYNIGYQGFYIPEGYKDRMYSFFRNFQPMSRQ
VVDEVNYKDFKAVAIPYQHNNSGFVGYMAPTMRQGQPYPANYPYPLIGTT
AVNSVTQKKFLCDRIMWRIPFSSNFMSMGALTDLGQNMLYANSAHALDMT
FEVDPMDEPTLLYLLFEVFDVVRVHQPHRGIIEAVYLRTPFSAGNATT

GenBank Accession No. AY803294 (SEQ ID NO: 202)

CATCATCAATAATATACCTTATAGATGGAATGGTGCCAATATGTAAATGAGGTGATTTTAAAAAGTGTGGGCTGTGT
GGTAATTGGCTGTGGGGTTAACGGCTAAAAGGGGCGGCGCGGCCGTGGGAAAATGACGTTTTTTGGGGGTGGAGTGT
TTTTGCAAGTTGTCGCGGTAAATGTGACGCAAACAAAGGCTTTTTTTTTACGGAACTACTTAGTGTTCCCACGGTAT
TTAACAGGAAATGAGGTAGTTTTGGCCGGATGCAAGTAAAAATTGTTCATTTTCGCGCGAAAACTGAATGAGGAAGT
GGTTTTCTGAATAATGCGGTATTTATGGCAGGGTGGAGTATTTGTTCAGGGCCAGGTAGACTTTGACCCATTACGTG
GAGGTTTCGATTACCGCGGAGGTTTCGATTACCGTGTTTTTTACCTAAATTTCCGCGTACCGTGTGAAAGTCTTCTG
TTTTTACGTAGGTGTCAGCTGATCGCTACGGTATTTATACCTCAGGGTTTGTGTCAAGAGGCCACTCTTGAGTGCCA
GCGAGAAGAGTTTTCTCCTCTGCGCCGGCAGTTTAATATTAAAAAAAATGAGACACTTGCGATTTATGCCTCAGGAA
ATAATTTCTGCTGAGACTGGAAACGAAATACTGGAGTTTGTGGTGCACGCCCTGATGGGAGACGATCCGGAGCCACC
TGTGCAGCTTTTTGAGCCTCCTACGCTTCAGGAACTGTATGATTTAGAGGTAGAGGGATCGGAGGATTCTAATGAGG
AAGCTGTGAATGGCTTTTTTACCGATTCTATGCTTTTAGCTGCTAATGAAGGATTAGAATTAGATCCGCCTTTGGAC
ACTTTCGATACTCCAGGGGTGATTGTGGAAAGCGGTACAGCTGTAAGAAAATTACCTGATTTGGGTTCCGTGGACTG
TGATTTGCACTGCTATGAAGACGGGTTTCCTTTGAGTGATGAGGAGGACCATGAAAAGGAGCAGTCTATGCAGACTG
CAGCGGGTGAGGGAGTGAAGGCTGCCATTGGTTTTCAGTTGGATTGCCCGGAGCTTCCTGGACATGGCTGTAAGTCT
TGTGAATTTCACAGGAAAAATACTGGAGTAAAGGAACTGTTATGTTCGCTTTGTTATATGAGAGCGCACTGCCACTT
TATTTACAGTAAGTGTGTTTAAGTTAAAATTTAAAGGAATATGCTGTTTTTCACATGTATATTGAGTGGGAAATTTG
TGCTTCTTATTATAGGTCCTGTGTCTGATGCTGATGAGTCACCATCTCCTGATTCTACTACCTCACCTCCTGAGATT
CAAGCACCTGTTCCTGTGGACGTGCACAAGCCCATTCCTGTAAAGCTTAAGCCTGGAAAACGTCCAGCAGTGGAAAA
ACTCGAGGACTTGTTACAGGGTGGGGACGGACCTTTGGACTTGAGTACACGGAAACGGCCAAGACAATAAGTGTTCC
ATATCCGTGTTTACTTAAGGTGACGTCAATATTTGTGTGAGAGTGCAATGTAATAAAAATATGTTAACTGTGTACTG
GTTTTTATTGCTTTTTGGGGGGGGACTCAGGTATATAAGTAGAAGCAGACCTGTGTGGTTAGCTCATAGAAGCTGGC
TTTGATTCATGGAGGTTTGGGCCATTTTGGAAGACCTTAGAAAGACTAGGCAACTGTTAGAGAACGCTTCGGACGGA
GTCTCCGGTTTTTGGAGATTCTGGTTCGCTAGTGAATTAGCTAGGGTAGTTTTTAGGATAAAACAGGACTATAAAGA
AGAATTTGAAAAGTTGTTGGTAGATTGTCCAGGACTTTTTGAAGCTCTTAATTTGGGCCATCAAGTTCACTTTAAAG
AAAAAGTTTTATCAGTTTTAGACTTTTCGACCCCAGGTAGAACTGCCGCTGCTGTGGCTTTTCTTACTTTTATATTA
GATAAATGGATCCCGCAGACTCATTTCAGCAGGGGATACGTTTTGGATTTCGTAGCCACAGCATTGTGGAGAACATG
GAAGGTTCGCAAGATGAGGACAATCTTAGGTTACTGGCCAGTGCAGCCTTTGGGTGTAGCGGGAATCCTGAGGCATC
CACCGGTCATGCCAGCGGTTCTGGAGGAGGAACAGCAAGAGGACAACCCGAGAGCCGGCCTGGACCCTCCAGTGGAG
GAGGCGGAGTAGCTGACTTGTCTCCTGAACTGCAACGGGTGCTTACTGGATCTACGTCCACTGGACGGGATAGGGGC
GTTAAAAGGGAGAGGGCATCTAGTGGTACTGATGCTAGATCTGAGTTGGCTTTAAGTTTAATGAGTCGCAGACGTCC
TGAAACCATTTGGTGGCATGAGGTCCAGAAAGAGGGAAGGGATGAAGTTTCTGTATTGCAGGAGAAATATTCACTGG
AACAGGTGAAAACATGTTGGTTGGAGCCTGAGGATGATTGGGAGGTGGCCATTAAAAATTATGCCAAGATAGCTTTG
AGGCCTGATAAACAGTATAAGATTACTAGACGGATTAATATCCGGAATGCTTGTTACATATCTGGAAATGGGGCTGA
GGTGGTAATAGATACTCCAGACAAGACAGTTATTAGATGCTGCATGATGGATATGTGGCCTGGAGTAGTCGGTATGG
AAGCAGTAACTTTTGTAAATGTTAAGTTTAGGGGAGATGGTTATAATGGAATAGTGTTTATGGCCAATACCAAACTT
ATATTGCATGGTTGTAGCTTTTTTGGTTTTAACAATACCTGTGTAGATGCCTGGGGACAGGTTAGTGTACGGGGATG
TAGTTTCTATGCGTGTTGGATTGCCACAGCTGGCAGAACCAAGAGTCAATTGTCTCTGAAGAAATGCATATTCCAAA
GATGTAACCTGGGCATTCTTAATGAAGGCGAAGCAAGGGTCCGCCACTGCGCTTCTACAGATACTGGATGTTTTATT
TTAATTAAGGGCAATGCCAGCGTAAAGCATAACATGATTTGCGGTGCTTCCGATGAGAGGCCTTATCAAATGCTCAC
TTGTGCCGGAGGGCATTGTAACATGCTGGCTACTGTGCATATTGTTTCTCATCAACGCAAAAAATGGCCTGTTTTTG
ATCACAATGTGTTGACCAAGTGTACCATGCATGCAGGTGGGCGTAGAGGAATGTTTATGCCTTACCAGTGTAACATG
AATCATGTAAAAGTGTTGTTGGAACCAGATGCCTTTTCCAGAATGAGTCTAACAGGAATGTTTGACATGAACATGCA
AATCTGGAAGATCCTGAGGTATGATGATACAAGATCGAGGGTGCGCGCATGCGAATGCGGAGGCAAGCATGCCAGGT
TCCAGCCGGTGTGTGTAGATGTGACTGAAGATCTGAGACCGGATCATTTGGTTATTGCCCGCACTGGAGCAGAGTTC
GGATCCAGTGGAGAAGAAACTGACTAAGGTGAGTATTGGGAAAACTTTGGGGTGGGATTTTCAGATGGACAGATTGA
GTAAAAATTTGTTTTTCTGTCTTGCAGCTGTCATGAGTGGAAACGCTTCTTTTAATGGGGGAGTCTTCAGCCCTTAT
CTGACAGGGCGTCTCCCATCCTGGGCAGGAGTTCGTCAGAATGTTATGGGATCTACTGTGGATGGAAGACCCGTCCA
ACCCGCCAATTCTTCAACGCTGACCTATGCTACTTTAAGTTCTTCACCTTTGGACGCAGCTGCAGCCGCCGCTGCCG
CCTCTGTTGCCGCTAACACTGTGCTTGGAATGGGTTACTATGGAAGCATCCTGGCTAATTCCACTTCCTCTAATAAC
CCTTCTACCCTGACTCAGGACAAGTTACTTGTCCTTTTGGCCCAGCTGGAGGCTTTGACCCAACGTCTGGGTGAACT
TTCTCAGCAGGTGGCCGAGTTGCGAGTACAAACTGAGTCTGCTGTCGGCACGGCAAAGTCTAAATAAAAAAAAAAAA
TTCCAGAATCAATGAATAAATAAACGAGCTTGTTGTTGATTTAAAATCAAGTGTTTTTTATTTCATTTTTCGCGCAC
GGTATGCCCTAGACCACCGATCTCGATCATTGAGAACACGGTGGATTTTTTCCAAAATCCTATAAAGGTGGGATTGA
ATGTTTAGATACATGGGCATTAGGCCGTCTTTGGGGTGGAGATAGCTCCATTGAAGGGATTCATGCTCCGGGGTAGT
GTTGTAAATTACCCAGTCATAACAAGGTCGCTGTGCATGGTGTTGCACAATATCTTTTAGAAGTAGGCTGATTGCCA
CAGATAAGCCCTTGGTGTAGGTGTTTACAAACCGGTTGAGCTGGGAGGGGTGCATTCGGGGTGAAATTATGTGCATT
TTGGATTGGATTTTTAAGTTGGCAATATTGCCGCCAAGATCTCGTCTTGGGTTCATGTTATGAAGTACCACCAAGAC
GGTGTATCCGGTACATTTAGGAAATTTATCGTGCAGCTTGGATGGAAAAGCGTGGAAAAATTTGGAGACACCCTTGT
GTCCTCCGAGATTTTCCATGCACTCATCCATGATAATAGCAATAGGGCCGTGGGCAGCAGCGCGGGCAAACACGTTC
CGTGGGTCTGACACATCATAGTTATGTTCCTGAGTTAAATCATCATAGGCCATTTTAATAAATTTGGGACGGAGAGT
ACCCGATTGGGGTATGAATGTTCCTTCGGGCCCCGGAGCATAGTTCCCCTCACAGATTTGCATTTCCCAAGCTTTCA
GTTCCGAGGGTGGAATCATGTCCACCTGGGGGGCTATAAAGAACACCGTTTCTGGGGCTGGGGTAATTAGTTGGGAT
GATAGCAAGTTTCTGAGCAATTGAGATTTGCCACATCCGGTGGGGCCATAAATGATTCCGATTACAGGTTGCAGTTG
GTAGTTTAGGGAACGGCAACTGCCGTCTTCTCGAAGCAAGGGGGCCACCTCGTTCATCATTTCCCTTACATGCATAT
TTTCCCGCACCAAATCCATTAGGAGGCGCTCTCCTCCTAGTGATAGAAGTTCTTGTAGTGAGGAAAAGTTTTTCAGC
GGTTTTAGACCGTCAGCCATGGGCATTTTGGAGAGAGTCTGTTGCAAAAGTTCTAGTCTGTTCCACAGTTCAGTGAT
GTGTTCTATGGCATCTCGATCCAGCAGACCTCCTCGTTTCGCGGGTTTGGACGGCTCCTGGAGTAGGGTATGAGACG
ATGGGCGTCCAGCGCTGCCAGGGTTCGGTCCTTCCAGGGTCTCAAAGTTCGGGTCAGGGTTGTTTCCGTCACAGTGA
AGGGGTGTGCGCCTGCTTGGGCGCTTGCCAGGGTGCGCTTCAGACTCATCCTGCTGGTCGAAAACTTGTGCCGCTTG
GCGCCCTGTATGTCGGCCAAGTAGCAGTTTACCATGAGTTCGTAGTTGAGCGCCTCGGCTGCGTGGCCCTTGGCGCG
GAGCTTACCTTTGGAAGTTTTCTTGCATACCGGGCAGTATAGGCATTTCAGCGCATACAGCTTGGGCGCAAGGAAAA
TGGATTCTGGGGAGTATGCATCCGCGCCGCAGGAGGCGCAAACAGTTTCACATTCCACCAGCCAGGTTAAATCCGGT
TCATTGGGGTCAAAAACAAGTTTTCCGCCATATTTTTTGATGCGTTTCTTACCTTTGGTCTCCATGAGTTCGTGTCC
TCGTTGAGTGACAAACAGGCTGTCCGTGTCCCCGTAGACTGATTTTACAGGCCTCTTTTCCAGTGGAGTGCCTCGGT
CTTCTTCGTATAGGAACTCTGACCACTCTGATACAAAGGCGCGCGTCCAGGCCAGCACAAAGGAGGCTATGTGGGAG
GGGTAGCGATCGTTGTCAACCAGGGGGTCCACCTTTTCCAAAGTATGCAAACACATGTCACTCTCTTCAACATCCAG
GAATGTGATTGGCTTGTAGGTGTATTTCACGTGACCTGGGGTCCCAGCTGGGGGGGTATAAAAGGGGGCGGTTCTCT
GCTCTTCCTCACTGTCTTCCGGATCGCTGTCCAGGAACGTCAGCTGTTGGGGTAGGTATTCCCTCTCGAAGGCGGGC
ATGACCTCTGCACTCAGGTTGTCAGTTTCTAAGAACGAGGAGGATTTGATATTGACAGTGCCGCTTGAGATGCCTTT
CATGAGGTTTTCGTCCATTTGGTCAGAAAACACAATTTTTTTATTGTCAAGTTTGGTGGCAAATGATCCATACAGGG
CGTTGGATAAAAGTTTGGCAATGGATCGCATGGTTTGGTTCTTTTCCTTGTCCGCGCGCTCTTTGGCAGCGATGTTG
AGTTGGACATATTCGCGTGCCAGGCACTTCCATTCGGGGAAGATAGTTGTCAATTCATCTGGCACAATTCTCACTTG
CCACCCTCGGTTATGCAAGGTAATTAAATCCACACTGGTGGCCACCTCGCCTCGAAGGGGTTCGTTGGTCCAGCAGA
GCCTACCTCCTTTCCTAGAACAGAAAGGTGGAAGTGGGTCTAGCATAAGTTCATCGGGAGGGTCTGCATCCATGGTA
AAGATTCCAGGAAGTAAATCCTTATCAAAATAGCTGATGGGAGTGGGGTCATCTAAGGCCATTTGCCATTCTCGAGC
TGCCAGTGCGCGCTCATATGGGTTAAGGGGACTGCCCCAGGGCATGGGATGGGTGAGTGCAGAGGCATACATGCCAC
AGATGTCATAGACGTAGATGGGATCCTCAAAGATGCCTATGTAGGTTGGATAGCATCGCCCCCCTCTGATACTTGCT
CGCACATAGTCATATAGTTCATGTGACGGCGCTAGCAGCCCCGGACCCAAGTTGGTGCGATTGGGTTTTTCTGTTCT
GTAGACAATCTGGCGAAAGATGGCGTGAGAATTGGAAGAGATGGTGGGTCTTTGAAAAATGTTGAAGTGGGCATGAG
GTAGACCTACAGAGTCTCTGATAAAGTGGGCATAAGATTCTTCAAGCTTGGTTACCAGTTGGGCGGTGACAAGTACG
TCCAGGGCGCAGTAGTCAAGTGTTTCTTGAATGATGTCATAACCTGGTTGGTTTTTCTTTTCCCACAGTTCGCGGTT
GAGAAGGTATTCTTCGCGATCCTTCCAGTACTCTTCTAGCGGAAACCCGTCTTTGTCTGCACGGTAAGATCCTAGCA
TGTAGAACTGATTAACTGCCTTGTAAGGGCAGCAGCCCTTCTCTACGGGTAGAGAGTATGCTTGAGCAGCTTTTCGT
AGCGAAGCGTGAGTAAGGGCGAAGGTGTCTCTAACCATGACTTTGACAAATTGGTATTTAAAGTCCATGTCGTCACA
GGCTCCCTGTTCCCAGAGTTGGAAGTCTACCCGTTTCTTGTAGGCGGGGTTGGGCAAAGCGAAAGTAACATCGTTGA
AGAGAATCTTACCGGCTCTGGGCATAAAATTGCGAGTGATGCGAAAAGGCTGTGGTACTTCCGCTCGATTGTTGATC
ACCTGGGCAGCTAGGACGATCTCGTCGAAGCCGTTGATGTTGTGTCCTACAATGTATAATTCTATGAAACGCGGCGT
GCCTCTGACGTGAGGTAGCTTATTGAGCTCATCAAAGGTTAGGTCTGTAGGGTCAGATAAGGCGTAGTGTTCAAGGG
CCCATTCGTGCAGATGAGGATTTGCATGTAGGAATGATGACCAAAGATCCACCGCCAGTGCTGTTTGTAACTGGTCC
CGATACTGACGAAAATGCTGGCCAATTGCCATTTTTTCTGGAGTGACACAGTAGAAGGTTCCGGGATCTTGTTGCCA
TCGATCCCACTTAAGTTTAATGGCTAGATCGTGGGCCATGTTGACGAGACGCTCTTCTCCTGAGAGTTTCATGACCA
GCATGAAAGGAACTAGTTGTTTGCCAAAGGACCCCATCCAGGTGTAAGTTTCCACATCGTAGGTCAGGAAGAGTCTT
TCTGTGCGAGGATGAGAGCCGATTGGGAAAAACTGGATTTCCTGCCACCAGTTGGAGGATTGGCTGTTGATGTGATG
GAAGTAGAAGTTTCTGCGGCGCGCCGAGCATTCGTGTTTGTGCTTGTACAGACGGCCGCAGTAGTCGCAGCGTTGCA
CGGGTTGTATCTCGTGAATGAGCTGTACCTGGCTTCCCTTGACGAGAAATTTCAGTGGGAAGCCGAGGCCTGGCGAT
TGTATCTCGTGCTCTTCTATATTCGCTGTATTGGCCTGTTCATCTTCTGTTTCGATGGTGGTCATGCTGACGAGCCC
CCGCGGGAGGCAAGTCCAGACCTCGGCGCGGGAGGGGCGGAGCTGAAGGACGAGAGCGCGCAGGCTGGAGCTGTCCA
GAGTCCTGAGACGCTGCGGACTCAGGTTAGTAGGTAGGGACAGAAGATTAACTTGCATGATCTTTTCCAGGGCGTGC
GGGAGGTTTAGATGGTACTTGATTTCCACAGGTTCGTTTGTAGAGACGTCAATGGCTTGCAGGGTTCCGTGTCCTTT
GGGTGCCACTACCGTACCTTTGTTTTTTCTTTTGATCGGTGGTGGCTCTCTTGCTTCTTGCATGCTTAAAAGCGGTG
ACGGGGACGCGCGCCGGGCGGCAGCGGTTGTTCCGGACCCGGGGGCATGGCTGGTAGTGGCACGTCGGCGCCGCGCA
CGGGCAGGTTCTGGTACTGCGCTCTGAGAAGACTTGCGTGCGCCACCACGCGTCGATTGACGTCTTGTATCTGACGT
CTTTGGGTGAAAGCTACCGGCCCCGTGAGCTTGAACCTGAAAGAGAGTTCAACAGAATCAATTTCGGTATCGTTAAC
GGCAGCTTGTCTCAGTATTTCTTGTACGTCACCAGAGTTGTCCTGGTAGGCGATCTCCGCCATGAACTGCTCGATTT
CTTCCTCCTGAAGATCTCCGCGACCCGCTCTCTCGACGGTGGCCGCGAGGTCATTGGAGATACGGCCCATGAGTTGG
GAGAATGCATTCATGCCCGCCTCGTTCCAGACGCGGCTGTAAACCACGGCCCCCTCGGAGTCTCTTGCGCGCATCAC
CACCTGAGCGAGGTTAAGCTCCACGTGTCTGGTGAAGACCGCATAGTTGCATAGGCGCTGAAAAAGGTAGTTGAGTG
TGGTGGCGATGTGTTCGGCGACAAAGAAATACATGATCCATCGTCTCAGCGGCATTTCGCTGACATCGCCCAGAGCT
TCCAAGCGCTCCATGGCCTCGTAGAAGTCCACGGCAAAATTAAAAAACTGGGAGTTTCGCGCGGACACGGTCAATTC
CTCCTCGAGAAGACGGATGAGTTCGGCTATGGTGGCCCGTACTTCGCGTTCGAAGGCTCCCGGCATCTCTTCTTCCT
CTTCTATCTCTTCTTCCACTAACATCTCTTCTTCGTCTTCAGGCGGGGGCGGAGGGGGCACGCGGCGACGTCGACGG
CGCACGGGCAAACGGTCGATGAATCGTTCAATGACCTCTCCGCGGCGGCGGCGCATGGTTTCAGTGACGGCGCGGCC
GTTCTCGCGCGGTCGCAGAGTAAAAACACCGCCGCGCATCTCCTTAAAGTGGTGACTGGGAGGTTCTCCGTTTGGGA
GGGAAAGGGCGCTGATTATACATTTTATTAATTGGCCCGTAGGGACTGCGCGCAGAGATCTAATCGTGTCAAGATCC
ACGGGATCTGAAAACCTTTCAACGAAAGCGTCTAACCAGTCACAGTCACAAGGTAGGCTGAGTACGGCTTCTTGTGG
GCGGGGGTGGTTATGTGTTCGGTCTGGGTCTTCTATTCCTTCTTCATCTCGGGAAGGTGAGACGATGCTGCTGGTGA
TGAAATTAAAGTAGGCAGTTCTAAGACGGCGGATGGTGGCGAGGAGCACCAGGTCTTTGGGTCCGGCTTGCTGGATA
CGCAGGCGATTGGCCATTCCCCAAGCATTATCCTGACATCTAGCCAGATCTTTGTAGTAGTCTTGCATGAGCCGTTC
TACGGGCACTTCTTCTTCACCCGTTCTGCCATGCATACGTGTGAGTCCAAACCCGCGCATTGGTTGGACCAGTGCCA
AGTCAGCTACAACTCTTTCGGCGAGGATGGCTTGCTGTACTTGGGTGAGGGTGGCTTGAAAGTCATCAAAATCCACG
AAGCGGTGGTAAGCCCCGGTATTGATGGTGTAAGCACAGTTGGCCATGACTGACCAGTTAACTGTTTGGTGACCATG
GCGCACGAGCTCGGTGTATTTAAGGCGCGAATAGGCGCGGGTGTCAAAAATGTAATCGTTGCAGGTGCGCACCAGAT
ACTGGTACCCTATAAGAAAATGCGGTGGTGGTTGGCGGTAGAGAGGCCATTGTTCTGTAGCTGGAGCGCCGGGGGCG
AGGTCTTCCAACATAAGGCGGTGATAGCCGTAGATGTACCTGGACATCCAGGTGATTCCTGCGGCGGTAGTGGAAGC
CCGAGGAAACTCGCGTACGCGGTTCCAAATGTTGCGTAGCGGCATGAAGTAGTTCATTGTAGGCACGGTTTGACCAG
TGAGGCGCGCGCAGTCATTGATGCTCTATAGACACGAAGAAAATGAAAGCGTTCAGCGACTCGACTCTGTAGCCTGG
AGGAACGTGAACGGGTTGGGTCGCGGTGTACCCCGGTTCAAGACTTGTACTCGAGCCGGCCGGAGCCGCGGCTAACG
TGGTATTGGCACTCCCGTCTCGACCCAGCCTACAAAAATCCAGGATACGGAATCGAGTCGTTTTGCTGGTTGCTGAA
TGGCAGGGAAGTGAGTCCTATTTTTTTTTTTGCCGCTCAGATGCATCCCGTGCTGCGACAGATGCGTCCCCAACAAC
AGCCCCCCTCGCAGCAGCAGCAACCACAAAAGGCTGTCCCTGCAACTACTGCAACTGCCGCCGTGAGCGGTGCGGGA
CAGCCCGCCTATGATCTGGACTTGGAAGAGGGCGAAGGACTGGCACGTCTAGGTGCGCCCTCGCCCGAGCGGCATCC
GCGAGTTCAACTGAAAAAAGATTCTCGCGAGGCGTATGTGCCCCAACAGAACCTATTTAGAGACAGAAGCGGCGAGG
AGCCGGAGGAGATGCGAGCTTCCCGCTTTAACGCGGGTCGTGAGCTGCGTCACGGTTTGGACCGAAGACGAGTGTTG
CGGGACGAGGATTTCGAAGTTGATGAAGTGACAGGGATCAGTCCTGCCAGGGCACACGTGGCTGCAGCCAACCTTGT
ATCGGCTTACGAGCAGACAGTAAAGGAAGAGCGTAACTTCCAAAAGTCTTTTAATAATCATGTGCGAACCCTGATTG
CCCGCGAAGAAGTTACTCTTGGTTTGATGCATTTGTGGGATTTGATGGAAGCTATCATTCAGAACCCTACTAGCAAA
CCTCTGACCGCACAGCTGTTTCTGGTGGTGCAACACAGCAGAGACAACGAGGCTTTCAGAGAGGCACTGCTCAACAT
CACTGAACCCGAGGGGAGATGGTTGTATGATCTTATCAACATTCTACAGAGTATCATAGTGCAGGAGCGGAGCCTGG
GCCTGGCCGAAAAGGTGGCTGCCATCAATTACTCGGTTTTAAGTTTGGGAAAATATTACGCTCGCAAGATCTACAAG
ACTCCATACGTTCCCATAGACAAGGAGGTGAAGATAGATGGGTTCTACATGCGTATGACGCTCAAGGTCTTGACCCT
GAGCGATGATCTTGGGGTGTACCGCAATGACAGAATGCATCGCGCCGTTAGCGCCAGTAGGAGGCGCGAGTTAAGCG
ACAGGGAACTGATGCACAGTTTGCAAAGAGCTCTGACTGGAGCTGGAACAGAGGGTGAGAATTACTTTGACATGGGA
GCTGACTTGCAGTGGCAGCCTAGTCGCAGGGCTCTGAGCGCCGCGACGGCAGGATGTGAGCTTCCTTACATAGAAGA
GGCGGATGAAGGCGAGGAGGAAGAGGGCGAGTACTTGGAAGACTGATGGCACAACCCGTGTTTTTTGCTAGATGGAA
CAGCAAGCACCGGATCCCGCAACGCGGGCGGCGCTGCAGAGCCAGCCGTCCGGCATTAACTCCTCGGACGATTGGAC
CCAGGCCATGCAACGTATCATGGCGTTGACGACTCGCAACCCCGAAGCCTTTAGACAGCAACCCCAGGCCAACCGTC
TATCGGCCATCATGGAAGCTGTAGTGCCTTCCCGCTCTAATCCCACTCATGAGAAGGTCCTGGCCATTGTAAACGCG
TTGGTGGAGAACAAAGCTATTCGTCCAGATGAGGCCGGACTGGTATACAACGCTCTTTTAGAACGCGTGGCTCGCTA
CAACAGTAGCAATGTGCAAACCAATTTGGACCGTATGATAACAGATGTACGCGAAGCCGTGTCTCAGCGTGAAAGGT
TCCAGCGCGATGCCAACCTTGGTTCGCTGGTGGCGTTAAATGCTTTTTTGAGTACTCAGCCTGCTAATGTGCCGCGT
GGTCAACAGGATTATACTAACTTTTTGAGTGCGTTGAGACTGATGGTATCTGAAGTACCTCAGAGCGAAGTGTATCA
GTCCGGACCTGACTACTTCTTTCAGACTAGCAGACAGGGTTTGCAGACGGTAAATCTGAGCCAAGCTTTTAAAAACC
TTAAAGGTTTGTGGGGAGTGCATGCCCCGGTAGGAGAAAGAGCAACCGTGTCTAGCTTGTTAACTCCAAACTCCCGC
CTATTACTACTGTTGGTAGCTCCTTTCACCGACAGCGGCAGCATCGACCGTAATTCCTATTTGGGTTACCTACTAAA
CCTGTATCGCGAAGCCATAGGGCAAAGCCAGGTGGACGAGCAGACCTATCAAGAAATTACCCAAGTCAGTCGCGCTT
TGGGTCAGGAAGACACTGGCAGTTTGGAAGCCACTCTGAACTTCTTGCTTACCAATCGGTCTCAGAAGATCCCTCCT
CAATATGCTCTTACTGCGGAGGAGGAGAGGATCCTTAGATATGTGCAGCAGAGCGTGGGATTGTTTCTGATGCAAGA
GGGGGCAACTCCGACTGCGGCATTGGACATGACAGCGCGAAATATGGAGCCCAGCATGTATGCCAGTAACCGGCCTT
TCATTAACAAACTGCTGGACTACTTGCACAGAGCTGCCGCTATGAACTCTGATTATTTTACCAATGCCATCCTAAAC
CCGCACTGGCTGCCCCCACCTGGTTTCTACACGGGCGAATATGACATGCCCGACCCTAATGACGGGTTTCTGTGGGA
CGACGTGGACAGTAATGTTTTTTCACCTCTTTTTGATCATCGCACGTGGAAAAAGGAAGGCGGCGATAGAATGCATT
CTTCTGCATCGCTGTCCGGGGTCATGGGTGCTACCGCGGCTGAGCCCGAGTCTGCAAGTCCTTTTCCTAGTCTACCC
TTTTCTCTACACAGTGTACGTAGCAGCGAAGTGGGTAGAATAAGTCGCCCGAGTTTAATGGGCGAAGAGGAATACCT
AAACGATTCCTTGCTCAGACCGGCGAGAGAAAAAAATTTCCCAAACAATGGAATAGAAAGTTTGGTGGATAAGATGA
GTAGATGGAAGACTTATGCTCAGGATCACAGAGACGAGCCTGGGATCATGGGGACTACAAGTAGAGCGAGCCGTAGA
CGCCAGCGTCATGACAGACAGAGGGGTCTTGTGTGGGAAGATGAGGATTCGGCCGATGATAGCAGCGTGTTGGACTT
GGGTGGGAGAGGAAGGGGCAACCCGTTTGCTCATTTGCGCCCTCGCTTGGGTGGTATGTTGTAAAAAAAAAATAAAA
AGGAAAACTCACCAAGGCCATGGCGACGAGCGTACGTTCGTTCTTCTTTATTATTTGTGTCTAGTATAATGAGGCGA
GTCGTGCTAGGCGGAGCGGTGGTGTATCCGGAGGGTCCTCCTCCTTCGTACGAGAGCGTGATGCAGCAGCAGCAGGC
GACGGCGGTGATGCAATCCCCACTGGAGGCTCCCTTTGTACCTCCGCGATACCTGGCACCTACGGAGGGCAGAAACA
GCATTCGTTACTCGGAACTGGCACCTCAGTACGATACCACCAGGTTGTATCTGGTGGACAACAAGTCGGCGGACATT
GCTTCTCTGAACTATCAGAATGACCACAGCAACTTCTTGACCACGGTGGTGCAGAACAATGACTTTACCCCTACGGA
AGCCAGTACCCAGACCATTAACTTTGATGAACGATCGCGGTGGGGCGGTCAGCTAAAGACCATCATGCATACTAACA
TGCCCAACGTAAACGAGTATATGTTTAGTAACAACTTCAAAGCGCGTGTGATGGTGTCCAGAAAACCTCCCGAAGGT
GCTGCAGTTGGGGATACATATGATCACAAGCAGGATATTTTGGAATATGAGTGGTTCGAGTTTACTTTGCCAGAAGG
CAACTTTTCAGTTACTATGACCATTGATTTGATGAACAATGCCATCATAGATAACTACTTGAAAGTGGGCAGACAGA
ATGGAGTGCTTGAAAGTGACATTGGTGTTAAGTTCGACACCAGGAACTTCAAGCTGGGATGGGATCCCGAAACCAAG
TTGATTATGCCTGGAGTGTATACGTATGAAGCCTTTCATCCTGACATTGTCTTACTGCCTGGCTGTGGAGTGGACTT
TACCGAAAGTCGTTTGAGCAACCTTCTTGGTATCAGAAAAAAACAGCCATTTCAAGAGGGTTTTAAGATTTTGTATG
AAGATTTAGAAGGAGGTAATATTCCGGCCCTCTTGGATGTAGATGCCTATGAGAACAGTAAGAAAGAACAAAAAGCC
AAAATAGAAGCTGCTGCGGAAGCTAAGGCAAACATAGTTGCCAGCGACTTTACAAGGGTTGCTAACGCTGGAGAGGT
CAGAGGAGACAATTTTGCACCAACACCTGTTCCGACTGCAGAATCATTATTGGCCGATGTAACTGGAGGAACGGACG
TGAAACTCACTATTCAACCTGTAGAAAAAGATAGTAAGAATAGAAGCTATAATGTGTTGGAAGATAAAATCAACACA
GCCTATCGCAGTTGGTACCTTTCGTACAATTATGGCGATCCCGAAAAAGGAGTGCGTTCCTGGACATTGCTCACCAC
CTCAGATGTCACCTGCGGAGCAGAGCAGGTCTACTGGTCGCTTCCAGACATGATGCAGGATCCTGTCACTTTCCGCT
CCACTAGACAAGTCAGCAACTACCCTGTGGTGGGTGCAGAGCTTATGCCCGTCTTCTCAAAGAGCTTCTACAACGAA
CAAGCTGTGTACTCCCAGCAGCTCCGCCAGTCCACCTCGCTTACGCACGTCTTCAACCGCTTTCCTGAGAACCAGAT
TTTAATCCGTCCGCCGGCGCCCACCATTACCACCGTCAGTGAAAACGTTCCTGCTCTCACAGATCACGGGACCCTGC
CGTTGCGCAGCAGTATCCGGGGAGTCCAACGTGTGACCGTTACTGACGCCAGACGCCGCACCTGTCCCTACGTGTAC
AAGGCACTGGGCATAGTCGCACCGCGCGTCCTTTCAAGCCGCACTTTCTAAAAAAATGTCCATTCTTATCTCGCCCA
GTAATAACACCGGTTGGGGTCTGCGCGCTCCAAGCAAGATGTACGGAGGCGCACGCAAACGTTCTACCCAACATCCC
GTGCGTGTTCGCGGTCATTTTCGCGCTCCATGGGGTGCCCTCAAGGGCCGCACTCGCGTTCGAACCACCGTCGATGA
TGTAATCGATGAGGTGGTTGCCGACGCCCGTAATTATACTCCTACTGCGCCTACATCTACTGTGGATGCAGTTATTG
ACAGTGTAGTGGCTGACGCTCGCAACTATGCTCGACGTAAGAGCCGGCGAAGGCGCATTGCCAGACGCCACCGAGCT
ACCACTGCCATGCGAGCCGCAAGAGCTCTGCTACGAAGAGCTAGACGCGTGGGACGAAGAGCCATGCTTAGGGCGGC
CAGACGTGCAGCTTCGGGCGCCAGCGCCGGCAGGTCCCGCAGGCAAGCAGCCGCTGTCGCAGCGGCGACTATTGCCG
ACATGGCCCAAACGCGAAGAGGCAATGTATACTGGGTGCGTGACGCTGCCACCGGTCAACGTGTACCCGTGCGCACC
CGTCCCCCTCGCACTTAGAAGATACTGAGCAGTCTCCGATGTTGTGTCCCAGCGGCGAGGATGTCCAAGCGCAAATA
CAAGGAAGAAATGCTGCAGGTTATCGCACCTGAAGTCTACGGCCAACCGCTGAAGGATGAAAAAAAACCCCGCAAAA
TCAAGCGGGCTAAAAAGGACAAAAAAGAAGAGGAAGATGGCGATGATGGGCTGGCGGAGTTTGTGCGCGAGTTTGCC
CCACGGCGACGCGTGCAATGGCGTGGACGCAAAGTTCGACATTTGTTGAGACCTGGAACTTCGGTGGTCTTTACACC
CGGCGAGCGTTCAAGCGCTACTTTTAAGCGTTCCTATGATGAGGTGTACGGGGATGATGATATTCTTGAGCAGGCGG
CTGACCGATTAGGCGAGTTTGCTTATGGCAAGCGTAGTAGAATAAATCCCAAGGATGAGACAGTGTCCATACCCTTG
GATCATGGAAATCCCACCCCTAGTCTTAAACCGGTCACTTTGCAGCAAGTGTTACCCGTAACTCCGCGAACAGGTGT
TAAACGCGAAGGTGAAGATTTGTATCCCACTATGCAACTAATGGTACCCAAACGCCAAAAGTTGGAGGACGTTTTGG
AGAAAGTAAAAGTGGATCCAGATATTCAACCTGAGGTTAAAGTGAGACCCATTAAGCAGGTAGCGCCTGGTCTGGGA
GTACAAACTGTAGACATTAAGATTCCCACTGAAAGTATGGAAGTGCAAACTGAACCCGCAAAGCCTACTGCCACCTC
CACTGAAGTGCAAACGGATCCATGGATGCCGATGCCTATTACAACTGACGCCGCCGGTCCCACTCGAAGATCCCGAC
GAAAGTACGGTCCAGCAAGTCTGTTGATGCCCAACTATGTTGTACACCCATCTATTATTCCTACTCCTGGTTACCGA
GGCACTCGCTACTATCGCAGCCGAAACAGTACCTCCCGCCGTCGCCGCAAGACACCTGCAAATCGCAGTCGTCGCCG
TAGACGCACAAGCAAACCGACTCCCGGCGCCCTGGTGCGGCAAGTGTACCGCAATAGTAGTGCGGAACCTTTGACAC
TGCCGCGTGCGCGTTACCATCCAAGTATCATCACTTAATCAATGTTGCCGCTGCCTCCTTGCAGATATGGCCCTCAC
TTGTCGCCTTCGCGTTCCCATCACTGGTTACCGAGGAAGAAATTCGCGCCGTAGAAGAGGGATGTTGGGGCGCGGAA
TGCGACGCTACAGGCGACGGCGTGCTATCCGCAAGCAATTGCGGGGTGGTTTTTTGCCAGCCTTAATTCCAATTATC
GCTGCTGCAATTGGCGCGATACCAGGCATAGCTTCCGTGGCGGTTCAGGCCTCGCAACGACATTGACATTGGAAAAA
AAAGTATAAATAAAAAAAAAAATACAATGGACTCTGACACTCCTGGTCCTGTGACTATGTTTTCTTAGAGATGGAAA
ACATCAATTTTTCATCCTTGGCTCCGCGACACGGCACGAAGCCGTACATGGGCACCTGGAGCGACATCGGCACGAGC
CAACTGAACGGGGGCGCCTTCAATTGGAGCAGTATCTGGAGCGGGCTTAAAAATTTTGGCTCAACCATAAAAACATA
CGGGAACAAAGCTTGGAACAGCAGTACAGGACAGGCGCTTAGAAATAAACTTAAAGACCAGAACTTTCAACAAAAAG
TAGTCGATGGGATAGCTTCCGGCATCAATGGAGTGGTAGATTTGGCTAATCAGGCTGTGCAGAAAAAGATAAACAGT
CGTTTGGACCCGCCGCCAGCAACCCCAGGTGAAATACAAGTGGAGGAAGAAATTCCTCCGCCAGAAAAACGAGGCGA
CAAGCGTCCGCGTCCCGATTTGGAAGAGACGCTGGTGACGCGCGTAGATGAACCGCCTTCTTATGAGGAAGCAACGA
AGCTTGGAATGCCCACCACTAGACCGATAGCCCCTATGGCTACCGGGGTAATGAAACCTTCTCAGTTGCATCGACCC
GTCACTTTGGATTTGCCCCCTCCCCCTGCTGCTACTGCTGTACCCGCTTCTAAGCCTGTCGCTGCCCCGAAACCAGT
CGCCGTAGCCAGGTCACGTCCCGGGGGCGCTCCTCGTCCAAATGCGCACTGGCAAAATACTCTGAACAGCATCGTGG
GTCTAGGCGTGCAAAGTGTAAAACGCCGTCGCTGCTTTTAATTAAATATGGAGTAGCGCTTAACTTGCCTATCTGTG
TATATGTGTCATTACACGCCGTCACAGCAGCAGAGGAAAAAAGGAAGAGGTCGTGCGTCGACGCTGAGTTACTTTCA
AGATGGCCACCCCATCGATGCTGCCCCAGTGGGCATACATGCACATCGCCGGACAGGATGCTTCGGAGTACCTGAGT
CCGGGTCTGGTGCAGTTCGCCCGCGCCACAGACACCTACTTCAATCTGGGAAATAAGTTTAGAAATCCCACCGTAGC
GCCAACCCACGATGTGACCACCGACCGTAGCCAGCGGCTCATGTTGCGCTTCGTGCCCGTTGACCGGGAGGACAATA
CATACTCTTACAAAGTGCGGTACACCCTGGCCGTGGGCGACAACAGAGTGCTGGATATGGCCAGCACGTTCTTTGAC
ATTAGGGGCGTGTTGGACAGAGGTCCCAGTTTCAAACCCTATTCTGGTACGGCTTACAACTCTCTGGCTCCTAAAGG
CGCTCCAAATGCATCTCAGTGGTTGGATAAAGGGGTTGAAACTACTGAAGAACGGCAAAATGAAGACGGGGAAAATG
ACGAAAAAGCTACATACACTTTTGGCAATGCCCCAGTAAAAGCCGATGCTGACATTACAAAAGACGGACTACCAATA
GGTTTGGAAGTCCCAGCTGAAGGTGACCCTAAACCTATCTACGCTAATAAGCTTTACCAACCAGAACCCCAGGTGGG
ACAGGAATCGTGGACTGATACAGATGGCACTGAAGAAAAATACGGAGGCAGAGTACTTAAACCGGACACTAAAATGA
AACCGTGCTATGGGTCTTTTGCTAAACCTACTAATGTGAAAGGCGGACAGGCAAAAGTGAAAACAGAAGAAGGCAAC
AACATTGAATATGACATTGACATGAACTTTTTTGACTTAAGATCACAAAAACAAGGTCTTAAACCTAAGATTGTAAT
GTATGCAGAAAATGTGGACCTGGAATCTCCAGATACTCATGTTGTGTACAAACCTGAAGTTTCAGATGCTAGTTCAA
ATGCTAATCTTGGACAGCAGTCTATGCCCAACAGACCCAACTACATTGGCTTCAGAGATAATTTTATTGGTCTTATG
TACTATAACAGTACTGGTAACATGGGGGTGCTGGCTGGCCAAGCATCTCAGTTGAATGCAGTGGTTGACTTGCAGGA
CAGAAACACAGAACTGTCTTACCAACTCTTGCTTGACTCCCTGGGCGATAGAACCAGATACTTTAGCATGTGGAATC
AGGCTGTTGACAGTTATGATCCCGATGTGCGTGTTATTGAAAATCATGGTGTGGAAGATGAACTTCCCAACTACTGT
TTTCCACTGGACGGCATCGGTCCGCGAACAGATAGTTACAAGGAGATTCAGTTAAATGGAGACCAAGCTTGGAAAGA
TGTAAATCCAAATGGTATCAGTGAACTTGTTAAGGGAAATCCATTTGCCATGGAAATTAACCTTCAAGCCAATCTAT
GGCGAAGTTTCCTTTATTCCAATGTGGCTCTGTATCTCCCAGACTCGTACAAATACACCCCGTCCAATGTCACTCTT
CCAGAAAACAAAAACACCTACGACTACATGAACGGGCGGGTGGTGCCGCCATCTCTAGTAGACACCTATGTGAACAT
TGGCGCCAGGTGGTCTCTGGATGCTATGGACAATGTCAACCCATTCAACCACCACCGTAACGCTGGCTTGCGTTACC
GATCCATGCTTTTGGGTAACGGACGTTATGTGCCTTTCCACATACAAGTGCCTCAAAAATTCTTCGCTGTCAAAAAC
CTGCTGCTTCTCCCAGGCTCCTACACTTATGAGTGGAACTTCAGGAAGGATGTGAACATGGTGCTACAGAGTTCCCT
CGGTAACGACCTACGGGTAGATGGCGCCAGCATCAGTTTCACGAGCATCAACCTCTATGCTACCTTTTTCCCCATGG
CTCACAACACCGCTTCCACCCTTGAAGCCATGCTGCGGAATGACACCAATGATCAGTCATTCAACGACTATCTATCT
GCAGCTAACATGCTCTATCCCATTCCTGCCAATGCAACCAATATTCCCATTTCCATTCCTTCTCGCAACTGGGCGGC
TTTCAGAGGCTGGTCATTTACCAGACTCAAAACCAAAGAAACTCCCTCTTTGGGGTCTGGATTTGACCCCTACTTTG
TCTATTCTGGTTCTATTCCCTACCTGGATGGTACCTTCTACCTGAACCACACTTTTAAGAAGGTTTCCATCATGTTT
GACTCTTCAGTGAGCTGGCCTGGAAATGACAGGTTACTATCTCCCAACGAATTTGAAATAAAGCGCACTGTGGATGG
CGAAGGCTACAATGTAGCCCAATGCAACATGACCAAAGACTGGTTCTTGGTACAGATGCTCGCCAACTACAACATAG
GCTATCAGGGCTTCTACATTCCAGAAGGATACAAAGATCGCATGTATTCATTTTTCAGAAACTTCCAGCCCATGAGC
AGGCAGGTGGTTGATGAGGTCAATTACAAAGACTTCAAGGCCGTCGCCATACCCTACCAACACAACAACTCTGGCTT
TGTGGGTTACATGGCTCCGACCATGCGCCAAGGTCAACCCTATCCCGCTAACTATCCCTATCCACTCATTGGAACAA
CTGCCGTAAATAGTGTTACGCAGAAAAAGTTCTTGTGTGACAGAACCATGTGGCGCATACCGTTCTCGAGCAACTTC
ATGTCTATGGGGGCCCTTACAGACTTGGGACAGAACATGCTTTATGCCAACTCAGCTCATGCTCTGGACATGACCTT
TGAGGTGGATCCCATGGATGAGCCCACCCTGCTTTATCTTCTCTTCGAAGTTTTCGACGTGGTCAGAGTGCATCAGC
CACATCGCGGCATCATCGAGACAGTCTACCTGCGTACACCGTTCTCGGCCGGTAACGCTACCACGTAAAAAGCTTCT
TGCTTCTTGCAAACAGCAGCTGCAACCATGGCCTGCGGATCCCAAAACGGCTCCAGCGAGCAAGAGCTCAGAGCCAT
TGTCCAAGACCTGGGTTGCGGACCCTATTTTTTGGGAACCTACGATAAGCGCTTCCCGGGGTTCATGGCCCCCGATA
AGCTCGCCTGTGCCATTGTAAACACGGCCGGACGTGAGACGGGGGGAGAGCACTGGTTGGCTTTCGGTTGGAACCCA
CGTTCTAACACCTGCTACCTTTTTGATCCTTTTGGATTCTCGGATGATCGTCTTAAACAGATTTACCAGTTTGAATA
TGAGGGTCTCCTGCGCCGCAGCGCTCTTGCTACCAAGGACCGCTGTATTACGCTGGAAAAATCTACCCAGACCGTGC
AGGGCCCCCGTTCTGCCGCCTGCGGACTTTTCTGCTGCATGTTCCTTCATGCCTTTGTGCACTGGCCTGACCGTCCC
ATGGACGGAAACCCCACCATGAAATTGCTGACTGGAGTGCCAAACAACATGCTTCATTCTCCTAAAGTCCAGCCCAC
CCTGTGTGACAATCAAAAAGCACTCTACCATTTTCTCAATACCCATTCGCCTTATTTTCGCTCCCATCGTACACACA
TCGAAAGGGCCACTGCGTTCGACCGTATGGATGTGCAATAATGACTCATGTAAACAACGTGTTGAATAAACAGCACT
TTATTTTTTACACGTATCAAGGCTCTGGATTACTTATTTATTTACAAGTCGAATGGGTTCTGACGAGAATCAGAATG
ACCCGCGGGCAGTGATACGTTGCGGAACTGATACTTGGGTTGCCACTTGAATTCGGGAATCACCAACTTGGGAACCG
GTATATCGGGTAGGATGTCACTCCACAGCTTTCTGGTCAGCTGCAAAGCTCCCAGCAGGTCAGGAGCCGAAATCTTG
AAATCACAATTAGGACCAGTGCTCTGAGCGCGAGAGTTGCGGTACACCGGATTGCAGCACTGAAACACCATCAGCGA
CGGATGTCTCACGCTTGCCAGCACGGTGGGATCTGCAATCATGCCCACATCCAGATCTTCAGCATTGGCAATGCTGA
ACGGGGTCATCTTGCAGGTCTGCCTACCCATGGCGGGCACCCAATTAGGCTTGTGGTTGCAATCGCAGTGCAGGGGG
ATTAGTATCATCTTGGCCTGATCCTGTCTGATTCCTGGATACACGGCTCTCATGAAAGCATCATATTGCTTGAAAGC
CTGCTGGGCTTTACTACCCTCGGTATAGAACATCCCGCAGGACCTGCTCGAAAACTGGTTAGCTGCGCAGCCGGCAT
CATTCACACAGCAGCGGGCGTCATTGTTGGCTATTTGCACCACACTTCTGCCCCAGCGGTTTTGGGTGATTTTGGTT
CGCTCGGGATTCTCCTTCAAGGCTCGTTGTCCATTCTCGCTGGCCACATCCATCTCGATAATCTGCTCCTTCTGAAT
CATAATAGTGCCATGCAGGCACTTCAGCTTGCCCTCATAATCATTGCAGCCATGAGGCCACAACGCACAGCCTGTAC
ATTCCCAATTATGGTGGGCGATCTGAGAAAAAGAATGTATCATTCCCTGCAGAAATCTTCCCATCATCGTGCTCAGT
GTCTTGTGACTAGTGAAAGTTAACTGGATGCCTCGGTGCTCCTCGTTTACGTACTGGTGACAGATGCGCTTGTATTG
TTCGTGCTGCTCAGGCATTAGTTTAAAAGAGGTTCTAAGTTCGTTATCCAGCCTGTACTTCTCCATCAGTACACACA
TCACTTCCATGCCCTTCTCCCAAGCAGACACCAGGGGCAAGCTAATCGGATTCTTAACAGTACAGGCAGCAGCTCCT
TTAGCCAGAGGATCATCTTTGGCAATCTTTTCAATGCTTCTTTTGCCATCCTTCTCAACGATGCGCACGGGCGGGTA
GCTGAAACCTACTGCTACAAGCTGCGCCTCTTCTCTTTCTTCTTCGCTGTCTTGACTGATGTCTTGCATGGGAACAT
GTTTGGTCTTCCTTGGCTTCTTTTTGGGGGGTATCGGGGGAGGAGGACTGTCGCTCCGTTCCGGAGACAGGGAGGAT
TGTGAAGTTTCGCTCACCATTACCAACTGACTGTCGGTAGAAGAACCTGACCCCACACGGCGACAGGTGTTTCTCTT
CGGGGGCAGAGGTGGAGGCGATTGCGAAGGGCTGCGGTCCGACCTGGAAGGCGGATGACTGGCAGAACCCCTTCCGC
GTTCGGGGGTGTGCTCCCTGTGGCGGTCGCTTAACTGATTTCCTTCGCGGCTGGCCATTGTGTTCTCCTAGGCAGAG
AAACAACAGACATGGAAACTCAGCCATTGCTGTCAACATCGCCACAAGTGCCATCACATCTCGTCGTCAGCGACGAG
GAAAAGGAGCAGAGCTTAACCATTCCACCGCCCAGTCCTGCCACCACCTCTACCCTAGAAGATAAGGAGGTCGACGC
ATCTCATGACATGCAGAATAAAAAAGCGAAAGAGTCTGAAACAGACATCGAGCAAGACCCGGGCTATGTGACACCGG
TGGAACACGAGGAAGAGTTGAAACGCTTTCTAGAGAGAGAGGATGAAAACTGCCCAAAACAGCAAGCGGATAACTAT
CACCAAGATGCTGGAAATAGGGATCAGAACACCGACTACCTCATAGGGCTTGACGGGGAAGACGCGCTCCTTAAACA
TCTAGCAAGACAGTCACTCATAGTCAAGGATGCATTATTGGACAGAACTGAAGTGCCCATCAGTGTGGAAGAGCTCA
GCCGCGCCTACGAGCTTAACCTTTTTTCACCTCGTACTCCCCCCAAACGCCAGCCAAACGGCACCTGCGAGCCAAAT
CCTCGCTTAAACTTTTATCCAGCTTTTGCTGTGCCAGAAGTACTCGCTACTTATCACATCTTTTTTAAAAATCAAAA
AATTCCAGTCTCCTGCCGCGCTAATCGCACCCGCGCTGACGCCCTACTTAATCTGGGACCTGGTTCACGCTTACCTG
ATATAGCTTCCTTGGAAGAGGTTCCAAAAATCTTCGAGGGTCTGGGCAATAATGAGACTCGGGCCGCAAATGCTCTG
CAAAAGGGAGAAAATGGCATGGATGAGCATCACAGCGTTCTGGTGGAATTGGAGGGCGATAATGCCAGACTCGCAGT
ACTCAAGCGAAGCGTCGAGGTCACACACTTTGCATACCCCGCTGTCAACCTGCCCCCTAAAGTTATGACGGCGGTCA
TGGACCAGTTACTCATTAAGCGCGCAAGTCCCCTTTCAGAAGACATGCATGACCCAGACGCCTGTGATGAGGGTAAA
CCAGTGGTCAGTGATGAGCAGCTAACCCGATGGCTGGACACCGACTCTCCCCGGGATTTGGAAGAGCGTCGCAAGCT
TATGATGGCCGTAGTGCTGGTTACCGTAGAACTAGAGTGTCTCCGGCGTTTCTTTACCGATTCAGAAACCTTGCGCA
AACTCGAAGAGAATCTGCACTACACTTTTAGACACGGCTTTGTGCGGCAGGCGTGCAAGATATCTAACGTGGAACTC
ACCAACCTGGTTTCCTACATGGGTATTCTGCATGAGAATCGTCTAGGACAAAGCGTGCTGCACAGCACCCTTAAGGG
GGAAGCCCGCCGTGATTACATCCGCGATTGTGTCTATCTCTACCTGTGCCACACGTGGCAAACCGGCATGGGTGTAT
GGCAGCAATGTTTAGAAGAACAGAACTTGAAAGAGCTTAACAAGCTCTTACAGAAATCTCTTAAGGTTCTGTGGACA
GGGTTCGACGAGCGCACCGTCGCTTCCGACCTGGCAGACCTCATCTTCCCAGAGCGTCTTAGGGTTACTTTGCGAAA
CGGACTGCCTGACTTTATGAGCCAGAGCATGCTTAACAATTTTCGCTCTTTCATCCTGGAACGCTCCGGTATCCTGC
CCGCCACCTGCTGCGCACTGCCCTCCGACTTTGTGCCTCTCACCTACCGCGAGTGCCCCCCGCCGCTATGGAGTCAC
TGCTACCTGTTCCGTCTGGCCAACTACCTCTCCTACCACTCGGATGTGATCGAGGATGTGAGCGGAGACGGCTTGCT
GGAGTGTCACTGCCGCTGCAATCTGTGCACGCCCCACCGGTCCCTAGCTTGCAACCCCCAGTTGATGAGCGAAACCC
AGATAATAGGCACCTTTGAACTGCAAGGCCCCAGCAGCCAAGGCGATGGGTCTTCTCCTGGGCAAAGTTTAAAACTG
ACCCCGGGACTGTGGACCTCTGCCTACTTGCGCAAGTTTGCCCCGGAAGATTACCACCCCTATGAAATCAAGTTCTA
TGAGGACCAATCACAGCCTCCAAAGGCCGAACTTTCGGCCTGCGTCATCACCCAGGGGGCAATTCTAGCCCAATTGC
AAGCCATCCAAAAATCCCGCCAAGAATTTCTACTGAAAAAGGGTAAGGGGGTCTACCTTGACCCCCAGACCGGCGAG
GAACTCAACACAAGGTTCCCTCAGGATGTCCCAACGACGAGAAAGCAAGAAGTTGAAGGTGCAGCCGCCGCCCCCAG
AAGATATGGAGGAAGATTGGGACAGTCAGGCAGAGGAAGCGGAGGAGGACAGTCTGGAGGACAGTCTGGAGGAAGAC
AGTTTGGAGGAGGAAAACGAGGAGGCAGAGGAGGTGGAAGAAGTAACCGCCGACAAACAGTTATCCCCGGCTGCGGA
GACAAGCAACAGCGCTATCATCTCCGCTCCGAGTCGAGGAACGCGGCGGCGTCCCAGCAGTAGATGGGACGAGACCG
GACGCTTCCCGAACCCAACCACCGCTTCCAAGACCGGTAAGAAGGATCGGCAGGGATACAAGTCCTGGCGGGGGCAT
AAGAATGCCATCATCTCCTGCTTGCATGAGTGCGGGGGAAACATATCCTTCACGCGACGCTACTTGCTATTCCACCA
TGGGGTGAACTTTCCACGCAATGTTTTGCATTACTACCGTCACCTCCACAGCCCCTACTATAGCCAGCAAATCCCGG
CAATCTCGACAGAAAAAGACAGCGGCGGCGACCTCCAACAGAAAACCAGCAGCGGCAGTTAAAAAATACACAACAAG
TGCAGCAACAGGAGGATTAAAGATTACAGCCAACGAGCCAGCGCAAACCCGAGAGCTAAGAAATCGGATCTTTCCAA
CCCTGTATGCCATCTTCCAGCAGAGTCGGGGCCAAGAGCAGGAACTGAAAATAAAAAACCGATCTTTGCGTTCGCTC
ACCAGAAGTTGTTTGTATCACAAGAGCGAAGATCAACTTCAGCGCACTCTTGAGGACGCCGAGGCTCTCTTCAACAA
GTACTGCGCGCTGACTCTTAAAGAGTAGGCAGCGACCGCGCTTATTCAAAAAAGGCGGGAATTACATCATCCTCGTC
ATGAGTAAAGAAATTCCCACGCCTTACATGTGGAGTTATCAGCCCCAAATGGGATTGGCGGCAGGCGCCTCCCAGGA
CTACTCCACCCGCATGAATTGGCTCAGCGCCGGGCCTTCTATGATTTCTCGAGTTAATGATATACGCGCCTACCGAA
ACCAAATACTTTTGGAACAGTCAGCTCTTACCACCACGCCCCGCCAACACCTTAATCCCAGAAATTGGCCCGCCGCC
CTAGTATACCAGGAAAGTCCCGCTCCCACCACTGTATTACTTCCTCGAGACGCCCAGGCCGAAGTCCAAATGACTAA
TGCAGGTGCGCAGTTAGCTGGCGGCTCCACCCTATGTCGTCACAGGCCTCGGCATAATATAAAACGCCTGATGATTA
GAGGCCGAGGTATTCAGCTTAACGACGAGTCGGTGAGCTCTCCGCTTGGTCTACGACCAGACGGAATCTTTCAAATT
GCCGGCTGCGGGAGATCTTCCTTCACCCCTCGTCAGACTGTTTTGACTTTGGAAAGTTCGTCTTCGCAACCCCGCTC
GGGCGGAATCGGGACCGTTCAATTTGTGGAGGAGTTTACTCCCTCTGTCTACTTCAACCCTTTCTCCGGATCTCCTG
GGCACTACCCGGACGAGTTCATACCGAACTTTGACGCAATTAGCGAGTCAGTGGACGGCTACGATTGATGTCTGGTG
ACGCGGCTGAGCTATCTCGGCTGCGACATCTAGACCACTGCCGCCGCTTTCGCTGTTTTGCCCGGGAACTCATTGAG
TTCATTTACTTCGAACTCCCCAAGGATCACCCTCAAGGTCCGGCCCACGGAGTGCGGATTACTATCGAAGGTAAAAT
AAACTCTCGCCTGCATCGAATTTTCTCCCAGCGGCCCGTGCTGATCGAGCGAGACCAGGGAAACACCACGGTTTCTA
TCTACTGCATTTGTAATCACCCAGGATTGCATGAAAGCCTTTGCTGTCTTATGTGTACTGAGTTTAATAAAAACTGA
ATTAAGACTCTCCTACGGACTGCCGCTTCTTCAACCCGGATTTTACAACCAGAAGAACGAAACTTTTCCTCTCATCC
AGGACTCTGTTAACTTTACCTTTCCTACTTACAAACCAGAAGCTCAACGACAACACCGCTTTTCCAGAAGCATTTTC
CCTACTAATACTACTTTCAAAACCGGAGGTGAGCTCCACAGTCTCCCCGCAGAAAACCCTTGGGTGGAAGCGGGCCT
TGTAGTGCTAGGAATTCTTGCGGGCGGGCTTGTGATTATTCTTTGCTACCTATACACACCTTGCTTCACTTTCCTAG
TGGTGTTGTGGTATTGGTTTAAAAAATGGGGCCCATACTAGTCTTGCTTGTTTTACTTTCGCTTTTGGGACCGGGTT
CTGCCAACTACAATCCATGTCTAGACTTTGACCCAGAAAACTGCACACTTACTTTTGCACCCGACACAAGCCGCATC
TGTGGAGTTCTTATTAAGTGCGGATGGGAATGCAGGTCCGTTGAAATTACACACAATAACAAAACCTGGAACAATAC
CTTATCCACCACATGGGAGCCAGGAGTTCCCGAGTGGTACACTGTCTCTGTCCGAGGTCCTGACGGTTCCATCCGCA
TTAGTAACAACACTTTTATTTTTTCTACAATGTGCGATCTGGCCATGTTCATGAGCAAACAGTATTCTCTATGGCCT
CCCAGCAAGGACAACATTGTAACGTTCTCCATTGCTTATTGCTTGTGCGCTTGCCTTCTTACTGCTTTACTGTGCGT
ATGCATACACCTGCTTGTAACCACTCGTATCAAAAACGCCAATAACAAAGAAAAAATGCCTTAACCTCTTTCTGTTT
ACAGACATGGCTTTTCTTACAGCTCTCATACTTGTCAGCATTGTCACTGCCGCTCACGGACAAACAGTCGTCTCTAT
CCCTCTAGGTCATAATTACACTCTCATAGGACCCCCAATCACTTCAGAGGTCATCTGGACCAAACTGGGAAGCGTTG
ATTACTTTGATATAATCTGTAACAAAACAAAACCAATAATAGTAACCTGCAACATACAAAATCTTACATTAATTAAT
GTTAGCAAAGTTTACAGCGGTTACTATTATGGTTATGACAGATACAGTAGTCAATATAGAAATTACTTGGTTCGTGT
TACCCAGTCCAAAACCACGAAAATGCCAAATATGGCAGAAATTCGATCCGATGACAATTCTCTAGAAACTTTTACAT
CTTCCACCACACCTGACGAAAAAAATATCCCAGATTCAATGATTGCAATTATCGCAGCGGTGGCAGTGGTGATGGCA
CTAACAGTAATATGCATGCTTTTATATGCTTGTCGCTACAAAAAGTTTCATCCTAAAAAACAAGATCTCCTACTAAG
GCTTAACATTTAATTTCTTTTTACACAGCCATGGTTTCCACTACCACATTCCTTATGCTTACTAGTATAGCAACTCT
GACTTCTGCTCGCTCACACCTCACTGTAACTATAGGCTCAAACTGCACACTAAAAGGACCTCAAGGTGGTCATGTCT
TTTGGTGGAGAATATATGACAATGGATGGTTTACAAAACCATGTGACCAACCTGGTAGATTTTTCTGCAACGGCAGA
GACCTAACCATTATCAACGTGACAGCAAATGACAAAGGCTTCTATTATGGAACCGACTATAAAAGTAGTTTAGATTA
TAACATTATTGTACTGCCATCCACCACTCCAGCGCCCCGCAAAACTACTTTCTCTAGCAGCAGTGCCGCTAACAATA
CAATTTCCAATCCAACCTTTACCGCGCTTTTAAAACGCACTGTGAATAATTCTACAACAATTTCCACTTCAACAATC
AGCATCATCGCTGCCGTGACAATTGGAATATCTATTCTTGTTTTTACCATAACCTACTACACCTGCTGCTATAAAAA
AGACGAACATAAAGGTGATCCATTACTTAGATTTGATATTTAATTTGTTCTTTTTTTTTTTATTTACAGTATGGTGA
ACACCAATCATGGTACCTAGAAATTTCTTCTTCACCATACTCATCTGTGCTTTTAATGTTTGCGCTACTTTCACAGC
AGTAGCCACAGCAAGCCCAGACTGTATAGGAGCATTTGCTTCCTATGCACTTTTTGCTTTTGTCACTTGCATCTGCG
TATGTAGCATAGTCTGCCTGGTTATTAATTTTTTCCAACTTCTAGACTGGATCCTTGTGCGAATTGCCTACCTGCGC
CACCATCCCGAATACCGCAACCAAAATATCGCGGCACTTCTTAGACTTATCTAAAACCATGCAGGCTATACTACCAA
TATTTTTGCTTCTATTGCTTCCCTACGCTGTCTCAACCCCAGCTACCTATAGTACTCCACCAGAACACCTTAGAAAA
TGCAAATTCCAACAACCGTGGTCATTTCTTGCTTGCTATCGAGAAAAATCTGAAATTCCCCCAACTTTAATAATGAT
TGCTGGAATAATTAATGTAATCTGTTGCACCATAATTTCATTTCTGATATACCCCCTATTTGATTTTGGCTGGAATG
CTCCCAATGCACATGATTATTCCCAAGACCCAGAGGAACACATTCCCCTACATAACATGCAACAACCAATAGCGCTA
ATAGAATACGAAAGTGAACCACAACCCCCACTACTCCCTGCTATTAGTTACTTCAACCTAACCGGCGGAGATGACTG
AAACACTCACCACCTCCAATTCCGCCGAGGATCTGCTTGATATGGACGGCCGCGTCTCAGAACAGCGACTCGCCCAA
CTACGCATCCGCCAGCAGCAGGAACGCGTGGCCAAAGAGCTCAGAGATGTCATCCAAATTCACCAATGCAAAAAAGG
CATATTCTGTTTGGTAAAACAAGCCAAAATATCCTACGAGATCACCGCTACCGACCATCGCCTCTCTTACGAGCTTG
GCCCCCAACGACAAAAATTTACCTGCATGGTGGGAATCAACCCCATAGTTATCACCCAACAAAGTGGAGATACTAAG
GGTTGCATTCACTGCTCCTGCGATTCCATCGAGTGCACCTACACCCTGCTGAAGACCCTATGCGGACTAAGAGACCT
GCTACCCATGAATTAAAAAAATGATTAATAAAAAATCACTTACTTAAAATCAGCAATAAGGTCTCTATTGAAATTTT
CTCCCAGCAGCACCTCACTTCCCTCTTCCCAACTCTGGTATTCTAAACCCCGTTCAGCGGCATACTTTCTCCATACT
TTAAAGGGGATGTCAAATTTTAGCTCCTCTCCTGTACCCACAATCTTCATCTCTTTCTTCCCAGATGACCAAGAGAG
TCCGGCTCAGTGACTCCTTCAACCCTGTCTACCCCTATGAAGATGAAAGCACCTCCCAACACCCCTTTATAAACCCA
GGGTTTATTTCCCCAAATGGCTTCACCCAAAGCCCAGACGGAGTTCTTACTTTAAAATGTTTAACCCCGCTAACAAC
CACAGGCGGGTCTCTACAGCTAAAAGTGGGAGGGGGACTTACAGTAGATGACACTGATGGGACCTTACAAGAAAACA
TAGGTGCCACCACACCACTTGTTAAGACTGGGCACTCTATAGGTTTATCCCTAGGAGCCGGATTGGGAACAGATGAA
AATAAACTTTGTACCAAATTGGGAGAAGGACTTACATTCAATTCAAACAACATTTGCATTGATGACAATATTAACAC
CCTGTGGACAGGAGTTAACCCCACCGAAGCCAACTGTCAAATGATGGACTCCAGTGAATCTAATGATTGCAAATTAA
TTCTAACACTAGTTAAAACTGGAGCCCTAGTCACTGCATTTGTTTATGTTATAGGAGTATCTAACAATTTTAATATG
CTAACTACATACAGAAATATAAATTTTACTGCGGAGCTGTTTTTTGATTCTGCGGGTAATTTACTAACTAGCCTGTC
ATCCCTAAAAACTCCACTTAATCATAAATCAGGACAAAACATGGCTACTGGTGCCATTACTAATGCTAAAAGTTTCA
TGCCCAGCACAACTGCTTATCCTTTCAATAATAATTCTAGAGAAAAAGAAAACTACATTTACGGAACTTGTCACTAC
ACAGCTAGTGATCACACTGCTTTTCCCATTGACATATCTGTCATGCTTAACCAAAGAGCAATAAGAGCTGATACATC
ATATTGTATTCGTATAACTTGGTCCTGGAACACAGGAGATGCCCCAGAGGGGCAAACCTCTGCTACAACCCTAGTTA
CCTCCCCATTTACCTTTTACTACATCAGAGAAGACGACTGACAAATAAAGTTTAACTTGTTTATTTGAAAATCAATT
CACAAACTCCGAGTAGTTATTTTGCCTCCCCCTTCCCATTTAACAGAATATACCAATCTCTCCCCACGCACAGCTTT
AAACATTTGGATACCATTAGAGATAGACATGGTTTTAGATTCCACATTCCAAACAGTTTCAGAGCGAGCCAATCTGG
GGTCAGTGATAGATAAAAATCCATCGGGATAGTCTTTTAAAGCGCTTTCACAGTCCAACTGATGCGGATGCGACTCC
GGAGTCTGGATCACGGTCATCTGGAAGAAGAACGATGGGAATCATAATCCGAAAACGGGATCGGGCGATTGTGTCTC
ATCAAACCCACAAGCAGCCGCTGTCTGCGTCGCTCCGTGCGACTGCTGTTTATGGGATCGGGATCCACAGTGTCCTG
AAGCATGATTTTAATAGCCCTTAACATTAACTTTCTGGTGCGATGCGCGCAGCAACGCATTCTTATTTCACTTAGAT
TAATACAGTAGGTACAGCACATTATCACAATATTGTTTAATAAACCATAATTAAAAGCACTCCAGCCAAAACTCATA
TCTGATATAATCGCCCCTGCATGACCATCATACCAAAGTTTAATATAAATTAAATGACGTTCCCTCAAAAACACACT
ACCCACATACATGATCTCTTTTGGCATGTGCATATTAACAATCTGTCTGTACCATGGACAACGTTGGTTAATCATGC
AGCCCAATATAACCTTCCGGAACCACACTGCCAACACCGCTCCCCCAGCCATGCATTGAAGTGAACCCTGCTGATTA
CAATGACAATGAAGAATCCAATTCTCTCGACCGTGAATCACTTGAGAATGAAAAATATCTATAGTAGCACAACATAG
ACATAAATGCATGCATCTTCTCATAATTTTTAACTCCTCAGGATTTAGAAACATATCCCAAGGAATAGGAAGCTCTT
GCAGAACAGTAAAGCTGGCAGAACAAGGAAGACCACGAACACAACTTACACTATGCATAGTCATAGTATCACAATCT
GGCAACAGCGGGTGGTCTTCAGTCATAGAAGCTCGGGTTTCATTTTCCTCACAACGTGGTAACTGGGCTCTGGTGTA
AGGATGATGTCTGGCGCATGATGTCGAGCGTGCGCACAACCTTGTCATAATGGAGTTGCTTCCTGACATTCTCGTAT
TTTGTATAGCAAAACGCGGCCCTGGCAGAACACACTCTTCTTCGCCTTTTATCCTGCCGCTTAGCGTGTTCCGTGTG
ATAGTTCAAGTACAGCCACACTCTTAAGTTGGTCAAAAGAATGCTGGCTTCAGTTGTAATCAAAACTCCATCACATC
TAATCGTTCTGAGGAAATGATCCACGGTAGCATATGCAAATCCCAACCAAGCAATGCAACTGGATTGCGTTTCAAGC
AGGAGAGGAGAGGGAAGAGACGGAAGAACCATGTTAATTTTTATTCCAAACGATCTCGCAGTACTTCAAATTGTAGA
TCGCGCAGATGGCATCTGTCGCCCCCACTGTGTTGGTGAAAAAGCACAGCTAGATCAAAAGAAATGCGATTTTCAAG
GTGCTCAACGGTGGCTTCCAGCAAAGCCTCCACGCGCACATCCAAGAACAAAAGAATACCAAAAGAAGGAGCATTTT
CTAACTCCTCAATCATCATATTACAGTCCTGCACCATTCCCAGATAATTTTCAGCTTTCCAGCCTTGGATTATTCGT
GTCATTTCTTGTGGTAAATCCAATCCACACATTACAAACAGGTCCCGGAGGGCGCCCTCCACCACCATTCTTAAACA
CACCCTCATAATGACAAAATATCTTGCTCCTGTGTCACCTGTAGCAAATTGAGAATGGCAACATCAATTGACATGCC
GTTGGCTCTAAGTTCTTCTCTAAGTTCTAGTTGTAAATACTCTTTCATATTATCACCAAACTGCTTAGCCAGAAGCC
CCCCGGGAACAAGAGCAGGACACGCTACAGTGCAGTACAAGCGCAGACCTCCCCAATTGGCTCCAGCAAAAACAAGA
TTGGAATAAGCATATTGGGAACCACCAGTAATGTCATCAAAGTTGCTGGAAATATAATCAGGCAGAGTTTGTTGTAA
AAATTGAATAAAAGAAAAATTTGCCAAAAAAACATTCAAAACCTCTGGGATGCAAATGCAATAAGTTACCGCGCTGC
GCTCCAACATTGTTAGTTTTGAATGAGTCTGCAAAAAATAAAAAAACAAGCGTCATATCAGAGTAGCCTGACGAACA
GGTGGATAAATCAGTCTTTCCATCACAAGACAAGCCACAGGGTCTCCAGCCCGACCCTCGTAAAACCTGTCATCGTG
ATTAAACAACAGCACCGAAAGTTCCTCGCGGTGACCAGCATGAATAATTCTTGATGAAGCATACAATCCAAACATGT
TAGCATCAGTTAAAGACAAAAAACAGCCAATATAGCCTCTGGGTATAATTATGCTTAATCGTAAATATAGCAAAGCC
ACCCCTCGCGGATACAAAGTAAAAGGCACAGGAGAATAAAAAATATAATTATTCCTTTGCTGCTGTTCAGGCAACGT
CGCCCCCGGTCCCTCTAAATACACATACAAAGCCTCATCAGCCATGGCTTACCAGACAAAGTACAGCAGGCACACAA
AGCACAAGCTCTAAAGTCACTCACCAACCTGTCCACAGTATATATACACAAACCCTAAACTGACGTAATGGGGCTAA
AGTACACAAAATCCCGCCAAACCCAACACACACCCCGAAACTGCGTCACCACAAAAGTACAGTTTCACTTCCGCAAT
CCCAACAAGCGGCACTTCCTCTTTCTCACGGGACGTCACATCCGCTTAACTTGCAACCTCATTTTCCCACGGCCGCG
CCGCCCCTTTTAGCCGTTAACCCCACAGCCAATTACCACACAGCCCACACTTTTTAAAATCACCTCATTTACATATT
GGCACCATTCCATCTATAAGGTATATTATTGATGATG

GenBank Accession No. AAW33140 (SEQ ID NO: 223)

MTKRVRLSDSFNPVYPYEDESTSQHPFINPGFISPNGFTQSPDGVLTLK
CLTPLTTTGGSLQLKVGGGLTVDDTDGTLQENIGATTPLVKTGHSIGLS
LGAGLGTDENKLCTKLGEGLTFNSNNICIDDNINTLWTGVNPTEANCQM
MDSSESNDCKLILTLVKTGALVTAFVYVIGVSNNFNMLTTYRNINFTAE
LFFDSAGNLLTSLSSLKTPLNHKSGQNMATGAITNAKSFMPSTTAYPFN
NNSREKENYIYGTCHYTASDHTAFPIDISVMLNQRAIRADTSYCIRITW
SWNTGDAPEGQTSATTLVTSPFTFYYIREDD

GenBank Accession No. AAW33119 (SEQ ID NO: 224)

MRRVVLGGAVVYPEGPPPSYESVMQQQQATAVMQSPLEAPFVPPRYLAP
TEGRNSIRYSELAPQYDTTRLYLVDNKSADIASLNYQNDHSNFLTTVVQ
NNDFTPTEASTQTINFDERSRWGGQLKTIMHTNMPNVNEYMFSNNFKAR
VMVSRKPPEGAAVGDTYDHKQDILEYEWFEFTLPEGNFSVTMTIDLMNN
AIIDNYLKVGRQNGVLESDIGVKFDTRNFKLGWDPETKLIMPGVYTYEA
FHPDIVLLPGCGVDFTESRLSNLLGIRKKQPFQEGFKILYEDLEGGNIP
ALLDVDAYENSKKEQKAKIEAAAEAKANIVASDFTRVANAGEVRGDNFA
PTPVPTAESLLADVTGGTDVKLTIQPVEKDSKNRSYNVLEDKINTAYRS
WYLSYNYGDPEKGVRSWTLLTTSDVTCGAEQVYWSLPDMMQDPVTFRST
RQVSNYPVVGAELMPVFSKSFYNEQAVYSQQLRQSTSLTHVFNRFPENQ
ILIRPPAPTITTVSENVPALTDHGTLPLRSSIRGVQRVTVTDARRRTCP
YVYKALGIVAPRVLSSRTF

GenBank Accession No. AAW33124 (SEQ ID NO: 225)

MATPSMLPQWAYMHIAGQDASEYLSPGLVQFARATDTYFNLGNKFRNPT
VAPTHDVTTDRSQRLMLRFVPVDREDNTYSYKVRYTLAVGDNRVLDMAS
TFFDIRGVLDRGPSFKPYSGTAYNSLAPKGAPNASQWLDKGVETTEERQ
NEDGENDEKATYTFGNAPVKADADITKDGLPIGLEVPAEGDPKPIYANK
LYQPEPQVGQESWTDTDGTEEKYGGRVLKPDTKMKPCYGSFAKPTNVKG
GQAKVKTEEGNNIEYDIDMNFFDLRSQKQGLKPKIVMYAENVDLESPDT
HVVYKPEVSDASSNANLGQQSMPNRPNYIGFRDNFIGLMYYNSTGNMGV
LAGQASQLNAVVDLQDRNTELSYQLLLDSLGDRTRYFSMWNQAVDSYDP
DVRVIENHGVEDELPNYCFPLDGIGPRIDSYKEIQLNGDQAWKDVNPNG
ISELVKGNPFAMEINLQANLWRSFLYSNVALYLPDSYKYTPSNVTLPEN
KNTYDYMNGRVVPPSLVDTYVNIGARWSLDAMDNVNPFNHHRNAGLRYR
SMLLGNGRYVPFHIQVPQKFFAVKNLLLLPGSYTYEWNFRKDVNMVLQS
SLGNDLRVDGASISFTSINLYATFFPMAHNTASTLEAMLRNDTNDQSFN
DYLSAANMLYPIPANATNIPISIPSRNWAAFRGWSFTRLKTKETPSLGS
GFDPYFVYSGSIPYLDGTFYLNHTFKKVSIMFDSSVSWPGNDRLLSPNE
FEIKRTVDGEGYNVAQCNMTKDWFLVQMLANYNIGYQGFYIPEGYKDRM
YSFFRNFQPMSRQVVDEVNYKDFKAVAIPYQHNNSGFVGYMAPTMRQGQ
PYPANYPYPLIGTTAVNSVTQKKFLCDRTMWRIPFSSNFMSMGALTDLG
QNMLYANSAHALDMTFEVDPMDEPTLLYLLFEVFDVVRVHQPHRGIIET
VYLRTPFSAGNATT

GenBank Accession No. AY601636 (SEQ ID NO: 203)

CATTATCTATAATATACCTTATAGATGGAATGGTGCCAACATGTAAATGAGGTAATTTAAAAAAGTGCGCGCTGTGT
GGTGATTGGCTGCGGGGTGAACGGCTAAAAGGGGCGGGCAATGCTGGGATGTGACGTAACTTATGTGGGAGGAGTTA
TGTTGCAAGTTATCGCGGTAAAGGTGACGTAAAACGAGGTGTGGTTTGGACACGGAAGTAGACAGTTTTCCCACGTT
TACTGACAGGATATGAGGTAGTTTTGGGCGGATGCAAGTGAAAATTCTCCATTTTCGCGCGAAAACTGAATGAGGAA
GTGAATTTCTGAGTCATTTCGCGGTTATGACAGGGTGGAGTATTTGCCGAGGGCCGAGTAGACTTTGACCGTTTACG
TGGAGGTTTCGATTACCGTGTTTTTCACCTAAATTTCCGCGTACGGTGTCAAAGTCCTGTGTTTTTACGTAGGTGTC
AGCTGATCACTAGGGTATTTAAACCTGTCGAGTTCCGTCAAGAGGCCACTCTTGAGTGCCAGCGAGAAGAGTTTTCT
CCTCCGCGCTGCGAGTCAGTTTTGCGCTTTGAAAATGAGACACCTGCGATTCCTGCCACAGGAGATTATCTCCAGCG
AGACCGGGATCGAAATACTGGAGTTTGTGGTAAATACCCTGATGGGAGATGACCCGGAACCGCCAGTGCAGCCTTTC
GATCCACCTACGCTGCACGATCTGTATGATTTAGAGGTAGACGGGCCTGATGATCCCAATGAGGAAGCTGTAAATGG
GTTTTTTACTGATTCTATGCTGCTAGCTGCCGATGAAGGATTGGACATAAACCCTCCTCCTGAGACCCTTGATACCC
CAGGGGTGGTTGTGGAAAGCGGCAGAGGTGGGAAAAAATTGCCTGATCTGGGAGCAGCTGAAATGGACTTGCGTTGT
TATGAAGAGGGTTTTCCTCCGAGTGATGATGAAGACGGGGAAACTGAACAGTCCATCCATACCGCAGTGAATGAGGG
AGTAAAAGCTGCCAGCGATGTTTTTAAGTTGGACTGTCCGGAGCTGCCTGGACATGGCTGTAAGTCTTGTGAATTTC
ACAGGAATAACACTGGAATGAAAGAACTATTGTGCTCGCTTTGCTATATGAGAATGCACTGCCACTTTATTTACAGT
AAGTGTATTTAAGTGAAATTTAAAGGAATAGTGTAGCTGTTTAATAACTGTTGAATGGTAGATTTATGTTTTTGCTT
GCGATTTTTTGTAGGTCCTGTGTCTGATGATGAGTCACCTTCTCCTGATTCAACTACCTCACCTCCTGAAATTCAGG
CGCCCGTACCTGCAAACGTATGCAAGCCCATTCCTGTGAAGCCTAAGTCTGGGAAACGCCCTGCTGTGGATAAGCTT
GAGGACTTGTTGGAGGGTGGGGATGGACCTTTGGACCTTAGTACCCGGAAACTGCCAAGGCAATGAGTGCCCTGCAG
CTGTGTTTATTTAGTGACGTCATGTAATAAAATTATGTCAGCTGCTGAGTGTTTTATTGCTTCTTGGGTGGGGACTT
GGATATATAAGTAGGAGCAGATCTGTGTGGTTAGCTCATAGCAACCTGCTGCCATCCATGGAGGTTTGGGCTATCTT
GGAAGACCTGAGACAGACTAGGCTACTGCTAGAAAACGCCTCGGACGGAGTCTCTGGCTTTTGGAGATTCTGGTTCG
GTGGCGATCTAGCTAGGCTAGTGTTTAGGATAAAACAGGACTATAGGGAAGAATTTGAAAAGTTATTGGACGACAGT
CCAGGACTTTTTGAAGCTCTTAACTTGGGCCATCAGGCTCATTTTAAGGAGAAGGTTTTATCAGTTTTAGATTTTTC
TACTCCTGGTAGAACTGCTGCTGCTGTAGCTTTTCTTACTTTTATATTGGATAAATGGATCCGCCAAACCCACTTCA
GCAAGGGATACGTTTTGGATTTCATAGCAGCAGCTTTGTGGAGAACATGGAAGGCTCGCAGGATGAGGACAATCTTA
GATTACTGGCCAGTGCAGCCTCTGGGAGTAGCAGGGATACTGAGACACCCACCGGCCATGCCAGCGGTTCTGGAGGA
GGAGCAGCAGGAGGACAATCCGAGAGCCGGCCTGGACCCTCCGGTGGAGGAGTAGCTGACTTGTTTCCTGAACTGCG
ACGGGTGCTTACTAGGTCTACGTCCAGTGGACAGGACAGGGGCATTAAGAGGGAAAGGAATCCTAGTGGGAATAATT
CAAGAACCGAGTTGGCTTTAAGTTTAATGAGCCGTAGGCGTCCTGAAACTGTTTGGTGGCATGAGGTTCAGAGCGAA
GGCAGGGATGAAGTTTCAATATTGCAGGAGAAATATTCACTAGAACAACTTAAGACCTGTTGGTTGGAACCTGAGGA
TGATTGGGAGGTGGCCATTAGGAATTATGCTAAGATATCTCTGAGGCCTGATAAACAGTATAGAATTACTAAGAAGA
TTAATATTAGAAATGCATGCTACATATCAGGGAATGGGGCAGAGGTTATAATAGATACACAAGATAAAGCAGCTTTT
AGATGTTGTATGATGGGTATGTGGCCAGGGGTTGTCGGCATGGAAGCAGTAACATTTATGAATATTAGGTTTAAAGG
GGATGGGTATAATGGCATTGTATTTATGGCTAACACTAAGCTGATTCTACATGGTTGTAGCTTTTTTGGGTTTAATA
ATACTTGTGTAGAAGCTTGGGGGCAAGTTAGTGTGAGGGGTTGTAGTTTTTATGCATGCTGGATTGCAACATCAGGT
AGGGTCAAGAGTCAGTTGTCTGTGAAGAAATGCATGTTTGAGAGATGTAATCTTGGCATACTGAATGAAGGTGAAGC
AAGGGTCCGCCACTGCGCAGCTACAGAAACTGGCTGCTTCATTCTAATAAAGGGAAATGCCAGTGTGAAGCATAATA
TGATCTGTGGACATTCGAATGAGAGGCCTTATCAGATGCTGACCTGCGCTGGTGGACATTGCAATATTCTGGCTACC
GTGCATATCGTTTCCCATGCACGCAAGAAATGGCCTGTATTTGAACATAATGTGATTACCAAGTGCACCATGCACAT
AGGTGGTCGCAGGGGAATGTTTATGCCTTACCAGTGTAACATGAATCATGTGAAGGTGATGTTGGAACCAGATGCCT
TTTCCAGAGTAAGCTTAACAGGAATCTTTGATATGAATATTCAACTATGGAAGATCCTGAGATATGATGACACTAAA
CCGAGGGTGCGCGCATGCGAATGCGGAGGCAAGCATGCTAGATTCCAGCCGGTGTGCGTGGATGTGACTGAAGACCT
GAGACCCGATCATTTGGTGCTTGCCTGCACTGGAGCGGAGTTCGGTTCTAGTGGTGAAGAAACTGACTAAAGTGAGT
AGTGGGACGAGCTGTGGAGGTGGGACTTTGAGGTTGGTAAGGTGGGCAGATTGGGTAAATTTTGTTAATTTCTGTCT
TGCAGCTGCCATGAGTGGAAGCGCTTCTTTTGAGGGGGGAGTATTTAGCCCTTATCTGACGGGCAGGCTCCCACCAT
GGGCAGGAGTTCGTCAGAATGTCATGGGATCCACTGTGGATGGGAGACCCGTCCAGCCCGCCAATTCCTCAACGCTG
ACCTATGCCACTTTGAGTTCGTCACCATTGGATGCAGCTGCAGCCGCCGCCGCTACTGCTGCCGCCAACACCATCCT
TGGAATGGGCTATTACGGAAGCATCGTTGCCAATTCCAGTTCCTCTAATAACCCTTCAACCCTGGCTGAGGACAAGC
TACTTGTTCTCTTGGCTCAGCTCGAGGCCTTAACCCAACGCTTAGGCGAACTGTCTAAGCAGGTGGCCCAGTTGCGT
GAGCAAACTCAGTCTGCTGTTGCCACAGCAAAGTCTAAATAAAGATCTTAAATCAATAAATAAAGAAATACTTGTTA
TAAAAACAAATGAATGTTTATTTGATTTTTCGCGCGCGGTATGCCCTGGACCATCGGTCTCGATCATTGAGAACTCG
GTGGATCTTTTCCAGTACCCTGTAAAGGTGGGATTGAATGTTTAGATACATGGGCATTAGTCCGTCCCGGGGGTGGA
GATAGCTCCATTGAAGAGCCTCTTGCTCCGGGGTAGTGTTATAAATCACCCAGTCATAGCAAGGTCGGAGTGCATGG
TGTTGCACAATATCTTTTAGGAGCAGACTAATTGCAACGGGGAGGCCCTTAGTGTAGGTGTTTACAAATCTGTTGAG
CTGGGACGGGTGCATCCGGGGTGAAATTATATGCATTTTGGACTGGATCTTGAGGTTGGCAATGTTGCCGCCTAGAT
CCCGTCTGGGGTTCATATTGTGCAGAACCACCAAGACAGTGTATCCGGTGCACTTGGGAAATTTATCATGCAGCTTA
GAGGGAAAAGCATGAAAAAATTTGGAGACGCCTTTGTGACCCCCCAGATTCTCCATGCACTCATCCATAATGATAGC
GATGGGGCCGTGGGCAGCGGCACGGGCGAACACGTTCCGGGGGTCTGAAACATCATAGTTATGCTCCTGAGTCAGGT
CATCATAAGCCATTTTAATAAACTTTGGGCGAAGGGTGCCAGATTGGGGGATGAAAGTTCCCTCTGGCCCGGGAGCA
TAGTTTCCCTCACATATTTGCATTTCCCAGGCTTTCAGTTCAGAGGGGGGGATCATATCCACCTGCGGGGCTATAAA
AAATACTGTTTCTGGAGCCGGGGTGATTAACTGGGATGAGAGCAAATTCCTAAGCAGCTGAGACTTGCCGCACCCAG
TGGGACCGTAAATGACCCCAATTACGGGTTGCAGATGGTAGTTTAGGGAGCGACAGCTGCCGTCCTCCCGGAGCAGG
GGGGCCACTTCGTTCATCATTTCCCTTACATGGATATTTTCCCGCACCAAGTCCGTTAGGAGGCGCTCTCCCCCAAG
GGATAGAAGCTCCTGGAGCGAGGAGAAGTTTTTCAGCGGCTTCAGCCCGTCAGCCATGGGCATTTTGGAAAGAGTCT
GTTGCAAGAGCTCGAGCCGGTCCCAGAGCTCGGTGATGTGCTCTATGGCATCTCGATCCAGCAGACCTCCTCGTTTC
GCGGGTTGGGACGGCTCCTGGAGTAGGGAATCAGACGATGGGCGTCCAGCGCTGCCAGGGTCCGATCCTTCCATGGT
CGCAGCGTCCGAGTCAGGGTTGTTTCCGTCACGGTGAAGGGGTGCGCGCCTGGTTGGGCGCTTGCGAGGGTGCGCTT
CAGACTCATCCTGCTGGTCGAGAACCGCTGCCGATCGGCGCCCTGCATGTCGGCCAGGTAGCAGTTTACCATGAGTT
CGTAGTTGAGCGCCTCGGCCGCGTGGCCTTTGGCACGGAGCTTACCTTTGGAAGTTTTATGGCAGGCGGGGCAGTAG
ATACATTTGAGGGCATACAGCTTGGGCGCGAGGAAAATGGATTCGGGGGAGTATGCATCCGCACCGCAGGAGGCGCA
GACGGTTTCGCACTCCACGAGCCATGTCAGATCCGGCTCATCGGGGTCAAAAACAAGTTTTCCGCCATGTTTTTTGA
TGCGTTTCTTACCTTTGGTTTCCATGAGTTCGTGTCCCCGCTGGGTAACAAAGAGGCTGTCCGTGTCCCCGTAGACT
GACTTTATGGGCCTGTCCTCGAGCGGAGTGCCGCGGTCCTCTTCGTAGAGGAACCCAGCCCACTCTGATACAAAAGC
GCGTGTCCAGGCCAGCACAAAGGAGGCCACGTGGGAGGGGTAGCGGTCGTTGTCAACCAGGGGGTCCACCTTCTCTA
CGGTATGTAAACACATGTCCCCCTCCTCCACATCCAAGAATGTGATTGGCTTGTAAGTGTAGGCCACGTGACCAGGG
GTCCCCGCCGGGGGGGTATAAAAGGGGGCGGGCCTCTGTTCGTCTTCACTGTCTTCCGGATCGCTGTCCAGGAGCGC
CAGCTGTTGGGGTAGGTATTCCCTCTCGAAGGCGGGCATGACCTCTGCACTCAGGTTGTCAGTTTCTAGGAACGAGG
AGGATTTGATATTGACAGTACCAGCAGAGATGCCTTTCATAAGACTCTCGTCCATCTGGTCAGAAAACACAATCTTC
TTGTTGTCCAGCTTGGTGGCAAATGATCCATAGAGGGCATTGGATAGAAGCTTGGCGATAGAGCGCATGGTTTGGTT
CTTTTCCTTGTCCGCGCGCTCCTTGGCGGCGATGTTAAGCTGGACGTACTCGCGCGCCACACATTTCCATTCAGGGA
AGATGGTTGTCAGTTCATCCGGAACTATTCTGACTCGCCATCCCCTATTGTGCAGGGTTATCAGATCCACACTGGTG
GCTACCTCGCCTCGGAGGGGCTCATTGGTCCAGCAGAGTCGACCTCCTTTTCTTGAACAGAAAGGGGGGAGGGGGTC
TAGCATGAACTCATCAGGGGGGTCCGCATCTATGGTAAATATTCCCGGTAGCAAATCCTTGTCAAAATAGCTGATGG
TGGCGGGATCATCCAAAGTCATCTGCCATTCTCGAACTGCCAGCGCGCGCTCATAGGGGTTAAGAGGGGTGCCCCAG
GGCATGGGGTGGGTGAGCGCGGAGGCATACATGCCACAGATATCGTAGACATAGAGGGGCTCTTCGAGGATGCCGAT
GTAAGTGGGATAACAGCGCCCCCCTCTGATGCTTGCTCGCACATAGTCATAGAGTTCATGTGAGGGGGCGAGAAGAC
CCGGGCCCAGATTGGTGCGGTTGGGTTTTTCCGCCCTGTAAACGATCTGGCGAAAGATGGCATGGGAATTGGAAGAG
ATAGTAGGTCTCTGGAATATGTTAAAATGGGCATGAGGGAGGCCTACAGAGTCCCTTATGAAGTGGGCATATGACTC
TTGCAGCTTGGCTACCAGCTCGGCGGTGACAAGTACGTCTAGGGCACAGTAGTCGAGAGTTTCCTGGATGATGTCAT
AACGCGGTTGGTTTTTCTTTTCCCACAGCTCGCGGTTGAGAAGGTATTCTTCGCGATCCTTCCAGTACTCTTCGAGG
GGAAACCCGTCTTTTTCTGCACGGTAAGAGCCCAACATGTAGAATTGATTGACTGCCTTGTAGGGACAGCATCCCTT
CTCCACTGGGAGAGAGTATGCTTGGGCTGCATTGCGCAGCGAGGTATGAGTGAGGGCAAAAGTGTCCCTGACCATGA
CTTTGAGGAATTGATACTTGAAGTCGATGTCATCACAGGCCCCCTGTTCCCAGAGTTGGAAGTCCACCCGCTTCTTG
TAGGCGGGGTTGGGCAAAGCGAAAGTAACATCATTGAAGAGGATCTTGCCGGCCCTGGGCATAAAATTTCGGGTGAT
TCTGAAAGGCTGAGGGACCTCTGCTCGGTTATTGATAACCTGAGCGGCCAAGACGATCTCATCAAAGCCATTGATGT
TGTGCCCCACTATGTACAGTTCTAAGAATCGAGGTGTGCCCCTGACATGAGGCAGCTTCTTGAGTTCTTCAAAAGTG
AGGTCTGTAGGGTCAGTGAGAGCATAGTGTTCGAGGGCCCATTCGTGCAGGTGAGGGTTCGCTTTGAGGAAGGAGGA
CCAGAGGTCCACTGCCAGTGCTGTTTGTAACTGGTCCCGGTACTGACGAAAATGCTGCCCGACTGCCATCTTTTCTG
GGGTGACGCAATAGAAGGTTTGGGGGTCCTGCTGCCAGCGATCCCACTTGAGTTTTATAGCCAGGTCATAGGCGATG
TTGACGAGCCGCTGGTCTCCAGAGAGTTTCATGACCAGCATGAAGGGGATTAGCTGCTTGCCAAAGGACCCCATCCA
GGTGTAGGTTTCCACATCGTAGGTGAGGAAGAGCCTTTCTGTGCGAGGATGAGAGCCAATCGGGAAGAACTGGATCT
CCTGCCACCAGTTGGAGGAATGGCTGTTGATGTGATGGAAGTAGAACTCCCTGCGACGCGCCGAGCATTCATGCTTG
TGCTTGTACAGACGGCCGCAGTACTCGCATCGATTCACGGGATGCACCTCATGAATGAGTTGTACCTGACTTCCTTT
GACGAGAAATTTCAGTGGAAAATTGAGGCCTGGCGCTTGTACCTCGCGCTCTACTATGTTGTCTGCATCGGCATGAC
CATCTTCTGTCTCGATGGTGGTCATGCTGACGAGCCCTCGCGGGAGGCAAGTCCAGACCTCGGCGCGGCAGGGGCGG
AGCTCGAGGACGAGAGCGCGCAGGTCGGAGCTGTCCAGGGTCCTGAGACGCTGCGGAGTCAGGTTAGTAGGCAGTGT
CAGGAGATTGACTTGCATGATCTTTTCGAGGGCGTGAGGGAGGTTCAGATGGTACTTGATCTCCACGGGTCCGTTGG
TGGAGATGTCGATGGCTTGCAGGGTTCCGTGCCCCTTGGGCGCTACCACCGTGCCCTTGTTTTTCCGTTTGGGCGGC
GGTGGCTCTGTTGCTTCTTGCATGTTTAGAAGCGGTGTCGAGGGCGCGCACCGGGCGGCAGGGGCGGCTCGGGACCC
GGCGGCATGGCCGGCAGTGGTACGTCGGCGCCGCGCGCGGGTAGGTTCTGGTACTGCGCCCTGAGAAGACTTGCATG
CGCGACGACGCGGCGGTTGACATCCTGGATCTGACGCCTCTGGGTGAACGCTACCGGCCCCGTGAGCTTGAACCTGA
AAGAGAGTTCAACAGAATCAATCTCGGTATCGTTGACGGCGGCTTGCCTAAGGATTTCTTGCACGTCGCCAGAGTTG
TCCTGGTAGGCGATCTCGGCCATGAACTGCTCGATCTCTTCCTCTTGAAGATCTCCGCGGCCCGCTCTCTCGACGGT
GGCCGCAAGGTCGTTGGAGATGCGTCCAATGAGTTGAGAGAATGCATTCATGCCCGCCTCGTTCCAGACGCGGCTGT
AGACCACAGCCCCCTCGGGATCTCTCGCGCGCATGACCACCTGGGCGAGGTTGAGCTCCACGTGGCGGGTGAAGACC
GCATAGTTGCATAGGCGCTGGAAAAGGTAGTTGAGTGTGGTGGCGATGTGCTCGGTGACGAAGAAATACATGATCCA
TCGTCTCAGCGGCATCTCGCTAACATCGCCCAGCGCTTCCAAGCGCTCCATGGCCTCGTAGAAGTCCACGGCAAAGT
TGAAAAACTGGGAGTTACGCGCGGACACGGTCAACTCCTCTTCCAGAAGACGGATGAGTTCGGCGATGGTGGTGCGC
ACCTCGCGCTCGAAAGCCCCCGGGATTTCTTCCTCAATTTCTTCTTCTTCCACTAACATCTCTTCCTCTTCAGGTGG
GGCTGCAGGAGGAGGGGGAACGCGGCGACGCCGGCGGCGCACAGGCAGACGGTCGATGAATCTTTCAATGACCTCTC
CGCGGCGGCGGCGCATGGTCTCGGTGACGGCACGACCGTTCTCCCTGGGTCTCAGAGTGAAGACGCCTCCGCGCATC
TCCCTGAAGTGGTGACTGGGAGGCTCTCCGTTGGGCAGGGACACCGCGCTGATTATGCATTTTATCAATTGCCCCGT
AGGTACTCCGCGCAAGGACCTGATCGTCTCAAGATCCACGGGATCTGAAAACCTTTCGACGAAAGCGTCTAACCAGT
CGCAATCGCAAGGTAGGCTGAGCACTGTTTCTTGCGGGCGGGGGCGGCTAGACGCTCGGTCGGGGTTCTCTCTTTCT
TCTCCTTCCTCCTCTTTGGAGGGTGAGACGATGCTGCTGGTGATGAAATTAAAATAGGCAGTTTTGAGACGGCGGAT
GGTGGCGAGGAGCACCAGGTCTTTGGGTCCGGCTTGTTGGATGCGCAGGCGATGGGCCATTCCCCAAGCATTATCCT
GACATCTGGCCAGATCTTTATAGTAGTCTTGCATGAGTCGTTCCACGGGCACTTCTTCTTCGCCCGCTCTGCCATGC
ATGCGAGTGATCCCGAACCCGCGCATGGGCTGGACAAGTGCCAGGTCCGCTACAACCCTTTCTGCGAGGATGGCTTG
CTGCACCTGGGTGAGGGTGGCTTGGAAGTCGTCAAAGTCCACGAAGCGGTGGTAAGCCCCGGTGTTGATTGTGTAGG
AGCAGTTGGCCATGACTGACCAGTTGACTGTCTGGTGCCCCGGACGCACAATCTCGGTGTACTTGAGGCGCGAGTAG
GCGCGGGTGTCAAAGATGTAATCGTTACAGGTGCGCACCAGGTACTGGTAGCCGATGAGAAAGTGAGGCGGCGGCTG
GCGGTATAGGGGCCATCGCTCTGTAGCCGGGGCGCCAGGGGCGAGGTCTTCCAGCATGAGGCGGTGATAACCGTAGA
TGTACCTGGACATCCAGGTGATACCGGAGGCGGTGGTGGATGCCCGCGGGAACTCGCGTACGCGGTTCCAGATGTTG
CGCAGCGGCATGAAGTAGTTCATGGTAGGCACGGTTTGGCCCGTGAGACGCGCACAGTCGTTGATGCTCTAGACATA
CGGGCAAAAACGAAAGCGGTCAGCGGCTCGTCTCCGTGGCCTGGAGGCTAAGCGAACGGGTTGGGCTGCGCGTGTAC
CCCGGTTCGAATCTCGGATCAGGCTGGAGCCGCAGCTAACGTGGTACTGGCACTCCCGTCTCGACCCAGGCCTGCAC
AAAACCTCCAGGATACGGAGGCGGGTCGTTTTTTTTTTGCTTTTTCCTGGATGGGAGCCAGTGCTGCGTCAAGCTTT
AGAACACTCAGTTCTCGGGGCTGGGAGTGGCTCGCGCCCGTAGTCTGGAGAATCAATCGCCAGGGTTGCGTTGCGGT
GTGCCCCGGTTCGAGTCTTAGCGCGCCGGATCGGCCGGTTTCCGCGACAAGCGAGGGTTTGGCAGCCTCGTCATTTC
TAAGACCCCGCCAGCCGACTTCTCCAGTTTACGGGAGCGAGCCCTCTTTTTTTTGTTTTTTGTTGCCCAGATGCATC
CCGTGCTGCGACAGATGCGCCCCCAGCAACAGCCCCCTTCTCAGCAGCAGCTACAACAACAGCCACAAAAGGCTCTT
CCTGCTCCTGTAACTACTGCGGCTGCAGCCGTCAGCGGCGCGGGACAGCCCGCCTATGATCTGGACTTGGAAGAGGG
CGAGGGATTGGCGCGCCTGGGGGCTCCATCGCCCGAGCGGCACCCGCGGGTGCAACTAAAAAAGGACTCTCGCGAGG
CGTACGTGCCCCAACAGAACCTATTCAGGGACAGGAGCGGCGAGGAGCCAGAGGAGATGCGAGCATCTCGATTTAAC
GCGGGTCGCGAGCTGCGCCACGGTCTGGATCGAAGACGGGTGCTGCAAGACGAGGATTTTGAGGTCGATGAAGTGAC
AGGGATCAGCCCAGCTAGGGCACATGTGGCCGCGGCCAACCTAGTCTCGGCCTACGAGCAGACCGTGAAGGAGGAGC
GCAACTTCCAAAAATCTTTTAACAACCATGTGCGCACCCTGATCGCCCGCGAGGAAGTGACCCTGGGTCTGATGCAC
CTGTGGGACCTGATGGAGGCTATCACCCAGAACCCCACTAGCAAACCCCTGACAGCTCAGCTGTTTCTGGTGGTTCA
ACATAGCAGGGACAACGAGGCATTCAGGGAGGCGTTGTTGAATATCACCGAGCCTGATGGGAGATGGCTGTATGATC
TGATTAACATCCTGCAAAGTATTATAGTGCAGGAACGTAGCCTGGGTTTGGCTGAGAAAGTGGCAGCTATCAACTAC
TCGGTCTTGAGCCTGGGCAAATACTACGCTCGCAAGATCTACAAGACCCCCTACGTACCCATTGACAAGGAGGTGAA
GATAGATGGGTTTTACATGCGCATGACTCTCAAGGTGTTGACTTTAAGCGACGATCTGGGGGTGTATCGCAATGACA
GGATGCACCGCGCGGTGAGCGCCAGCAGGAGGCGCGAGCTGAGCGACAGAGAACTTATGCACAGCTTGCAAAGGGCT
CTAACGGGGGCCGGAACTGATGGGGAGAACTACTTTGACATGGGAGCGGACTTGCAATGGCAACCCAGTCGCAGGGC
CATGGAGGCTGCAGGGTGTGAGCTTCCTTACATAGAAGAGGTGGATGAAGTCGAGGACGAGGAGGGCGAGTACTTGG
AAGACTGATGGCGCGACCCGTATTTTTGCTAGATGGAACAACAGCAGGCACCGGACCCCGCAATGCGGGCGGCGCTA
CAGAGCCAGCCGTCCGGCATTAACTCCTCGGACGATTGGACCCAGGCCATGCAACGTATAATGGCGCTGACGACCCG
CAACCCCGAAGCCTTTAGACAGCAACCCCAGGCCAACCGCCTTTCGGCCATACTGGAGGCCGTAGTGCCCTCCCGCT
CCAACCCCACCCACGAGAAGGTCCTGGCTATCGTGAACGCGCTGGTGGAGAACAAGGCCATCCGTCCCGATGAGGCC
GGGCTGGTATACAATGCTCTTTTGGAGCGCGTGGCCCGTTACAACAGCAGCAACGTGCAGACCAACCTGGACCGGAT
GGTGACCGATGTGCGCGAGGCTGTGTCTCAGCGCGAGCGGTTCCAGCGCGACGCCAACTTGGGGTCATTGGTAGCGC
TAAACGCTTTCCTTAGCACCCAGCCCGCCAACGTGCCCCGTGGTCAGCAAGACTATACAAACTTTTTGAGTGCATTG
AGACTCATGGTATCTGAGGTGCCCCAGAGCGAGGTGTACCAGTCCGGGCCAGATTACTTCTTCCAGACCAGCAGACA
GGGCTTGCAGACAGTGAACCTGACCCAGGCTTTCAAGAACCTGAAGGGTCTGTGGGGAGTGCACGCCCCAGTAGGAG
ATCGCGCGACCGTGTCTAGCTTGCTGACTCCCAACTCCCGCCTGCTGCTGCTGCTGGTATCCCCCTTCACTGACAGC
GGTAGCATCGACCGCAACTCCTACTTGGGCTACCTGCTTAACCTGTATCGCGAGGCTATAGGACAGAGCCAGGTGGA
CGAGCAGACCTATCAAGAAATCACCCAAGTGAGCCGCGCCCTGGGTCAGGAAGACACAGGCGGTTTGGAAGCCACCC
TGAACTTCTTACTAACCAACCGGTCGCAGAAGATCCCTCCTCAGTATGCGCTTACCGCTGAGGAGGAGCGGATCCTA
AGATACGTGCAACAGAGCGTTGGACTGTTTTTGATGCAGGAGGGGGCGACACCTACCGCCGCGCTGGATATGACAGC
TCGAAACATGGAGCCCAGCATGTATGCTAGTAACAGGCCTTTCATTAACAAACTGCTGGACTACCTGCACAGGGCGG
CCGCCATGAACTCTGATTATTTCACCAATGCTATTCTGAACCCACACTGGCTGCCCCCACCTGGTTTCTACACTGGC
GAATACGACATGCCCGATCCCAATGACGGGTTCCTATGGGACGATGTGGACAGTAGCATATTTTCCCCGCCGCCAGG
TTATACGGTTTGGAAGAAGGAAGGGGGCGATAGAAGGCACTCTTCCGTATCGTTGCCCGGAACGGCTGGTGCTGCCG
CGGCCGTGCCCGAAGCTGCGAGTCCTTTCCCTAGCTTGTCCTTTTCACTAAACAGCGTTCGCAGCAGTGAACTGGGG
AGAATAAACCGCCCGCGCTTGATGGGCGAGGATGAGTACTTGAATGACTCTTTGCTGAGGCCAGAGAGGGAAAAGAA
CTTCCCTAACAATGGAATAGAGAGCCTGGTGGATAAGATGAGTAGATGGAAGACCTATGCGCAGGATCACAGAGACG
AGCCCAGGATCTTGGGGGCTACAAGCAGACCGAGCCGTAGACGCCAGCGCCACGACAGGCAGATGGGTCTTGTGTGG
GACGACGAGGACTCTGCCGATGACAGCAGCGTGTTGGACTTGGGTGGAAAAGGAGTTGGCAACCCGTTCGCTCATCT
GCGTCCCCGTTTCGGTCGCATGTTGTAAAAGTGAAAGTAAAAATAAAAAGGCAACTCACCAAGGCCATGGCAACCGA
GCGTGCGTTCGTTCTTTTTTTTGTTATCTGTATCTAGTACGATGAGGAGACGAGCCGTGCTAGGCGGAGCGGTGGTG
TATCCGGAGGGTCCTCCTCCTTCTTACGAGAGCGTGATGCAGCAACAGGCGGCGATGATACAGCCCCCACTGGAGGC
TCCCTTCGTACCCCCTCGGTACCTGGCGCCTACGGAAGGGAGAAATAGCATTCGTTACTCGGAGCTGTCACCCCAGT
ACGATACCACCAAGTTGTATCTGGTGGACAACAAGTCGGCGGACATCGCCTCCCTGAACTATCAGAACGACCACAGC
AACTTCCTGACCACAGTGGTGCAGAACAATGACTTTACCCCCACGGAGGCTAGCACCCAGACCATTAACTTTGACGA
GCGGTCGCGGTGGGGCGGTCAGCTGAAGACCATTATGCACACCAACATGCCCAACGTGAACGAGTACATGTTCAGCA
ACAAGTTTAAGGCGAGGGTGATGGTATCTAGGAAGGCTCCTGAAGGTGTTACAGTAAATGATCATAAAGATGATATT
TTGAAATATGAGTGGTTTGAGTTCACTTTACCAGAAGGTAACTTCTCAGCTACCATGACCATCGACCTGATGAACAA
TGCCATCATTGACAACTACCTGAAAATTGGCAGACAGAATGGAGTGCTGGAAAGTGACATTGGTGTTAAGTTTGACA
CTAGAAACTTCAGGCTCGGGTGGGACCCCGAAACTAAGTTGATTATGCCAGGGGTCTACACTTATGAGGCATTCCAT
CCTGACATTGTTTTGTTGCCTGGTTGCGGGGTAGATTTTACTGAAAGCCGACTTAGCAACTTGCTTGGCATCAGGAA
GAGACATCCATTCCAGGAGGGTTTCAAAATCATGTATGAAGATCTTGAAGGGGGTAATATTCCTGCCCTTTTGGATG
TCACTGCCTATGAGGAAAGCAAAAAGGATACCACTACTGAAACAGGCGAAAAGGCGGTGGTTAAAACAACCACAGTG
GCTGTTGCAGAGGAAACCAGTGAAGATGATAATATAACTAGAGGAGATACTTATATAACTGAAAAACAAAAACGTGA
AGCTGCAGCTGCAGAACTATTACTTATGTCTGAAGTTAAAAAAGAGTTAAAGATCCAACCTTTAGAAAAAGACAGCA
AGAATAGAAGCTACAATGTCTTGGAAGACAAAATCAACACAGCCTACCGCAGCTGGTACCTGTCCTACAATTATGGT
AACCCTGAGAAAGGAATAAGGTCCTGGACACTGCTCACCACTTCGGATGTCACCTGTGGAGCCGAGCAGGTCTACTG
GTCGCTCCCCGACATGATGCAAGACCCCATCACCTTCCGCTCCTCGAGACAAGTCAACAACTACCCAGTAGTGGGTG
CAGAGCTTATGCCGGTCTTCTCAAAGAGTTTCTACAATGAGCAAGCCGTGTACTCTCAGCAGCTCCGACAGTCCACC
TCGCTCACGCACGTCTTCAACCGCTTCCCTGAGAACCAGATCCTCATCCGCCCGCCGGCGCCCACAATTACCACCAT
CAGTGAAAACGTTCCTGCTCTCACAGATCACGGGACCCTGCCGTTACGCAGCAGTATCCGGGGAGTCCAGCGCGTGA
CCGTTACTGACGCCAGACGTCGCACCTGTCCCTACGTTTACAAGGCCCTGGGCATAGTCGCGCCGCGCGTTCTTTCA
AGCCGCACTTTCTAAAAAAAAAAAAAAATGTCCATTCTCATCTCGCCCAGTAATAATACCGGTTGGGGACTGCATGC
GCCCACCAAGATGTACGGAGGCGCCCGCAAACGCTCTACCCAGCACCCCGTGCGCGTTCGCGGTCATTTCCGCGCTC
CCTGGGGCGCCCTCAAGGGCCGTACCCGCACTCGGACCACGGTCGATGATGTGATCGACCAGGTGGTTGCCGATGCT
CGTAATTATACTCCTACTGCGCCTACATCTACTGTGGATGCAGTTATTGACAGTGTAGTGGCAGACGCTCGCGCCTA
TGCTCGCCGGAAGAGCCGAAGGAGGCGCATCGCCAGGCGCCACAGGGCTACTCCCGCTATGCGAGCTGCAAAAGCTA
TTCTGCGGAGGGCCAAACGTGTGGGACGAAGAGCCATGCTTAGAGCGGCCAGACGCGCGGCTTCTGGTGCTAGCAGC
GGCAGGTCCCGCAGGCGCGCGGCCACGGCGGCAGCAGCGGCCATTGCCAACATGGCCCAACCGCGAAGAGGCAATGT
GTATTGGGTGCGCGATGCCACTACCGGCCAGCGCGTGCCCGTGCGCACCCGCCCCCCTCGCACTTAGAAGATACTGA
GCAGTCTCCGATGTTGTGTCCCAGCGGCAAGTATGTCCAAGCGCAAATACAAGGAAGAGATGCTCCAGGTCATCGCG
CCTGAAATCTACGGTCCGCCGATGAAGGATGAAAAAAAGCCCCGCAAAATCAAGCGGGTTAAAAAGGACAAAAAAGA
AGAAGATGGCGATGATGGACTGGTGGAGTTTGTGCGCGAGTTCGCGCCAAGACGGCGCGTGCAGTGGCGCGGTCGAA
AAGTACGCCAAGTGCTTAGACCCGGGACCACTGTGGTCTTTACACCTGGCGAGCGTTCCAGCACTACTTTTAAACGG
TCCTATGATGAGGTGTATGGGGATGACGATATTATTGAGCAGGCGGCAGACCGCCTTGGCGAGTTTGCTTATGGCAA
GCGCACAAGATCCAGTCCCAAAGAGGAGGCGGTATCTATTCCCTTGGATCATGGAAATCCCACCCCTAGCCTCAAAC
CAGTCACCCTGCAGCAAGTGCTGCCCGTACCTGCGAGCAGAGGCGTAAAGCGCGAGGGTGAGGACCTATATCCCACC
ATGCAGCTAATGGTGCCCAAGCGGCAGAGATTAGAAGACGTACTGGAGAAAATGAAAGTGGATGCCGATATCCAGCC
TGAGGTGAAAGTGAGACCCATCAAGGAAGTGGCGCCAGGTTTGGGAGTACAAACCTTTGACATCAAGATTCCCACTG
AGTCCATGGAAGTGCAGACCGAACCTGCAAAACCCACAGTCACCTCAATTGAGGTTCAGACGGAACCCTGGATGCCC
GCGCCCGTTGCCGCCCCCAGCACCACTAGAAGATCACGTCGAAAGTATGGCCCAGCAAGTCTGCTAATGCCCAACTA
TGCTCTGCACCCATCCATCATTCCCACTCCGGGTTACAGAGGCACTCGCTACTATCGAAGTCGGAGCAACACCTCAC
GCCGCCGCAAACTACCTGCAAGTCGCACTCGCCGTCGCCGCCGCCGCACCACTGCCAGCAAATTAACTCCCGCCGCC
CTGGTGCGGAGAGTGTACCGCGATGGTCGCGCTGAACCTCTGACGCTGCCGCGCGCGCGCTATCATCCAAGCATCAC
CACTTAATGACTGTTGACGCTGCCTCCTTGCAGATATGGCTCTCACTTGCCGCCTTCGCGTCCCCATTACTGGCTAC
CGAGGAAGAAACTCGCGCCGTAGAAGGATGTTGGGGCGAGGGATGCGCCGCCACAGACGAAGGCGCGCTATCAGCAA
GCGATTAGGGGGTGGCTTTCTGCCAGCTCTTATACCCATCATCGCCGCGGCGATCGGGGCGATACCAGGCATAGCTT
CCGTGGCGGTTCAGGCCTCGCAGCGCCACTAACAATGGAAAAATTTATAAATAAAAAATAGAATGGACTCTGACGCT
CCTGGTCCTGTGACTATGTTTTTGTAGAGATGGAAGACATCAATTTTTCATCCCTGGCTCCGCGACACGGCACGAGG
CCGTACATGGGCACCTGGAGCGACATCGGCACGAGCCAACTGAACGGGGGCGCCTTCAATTGGAGCAGTATCTGGAG
CGGGCTTAAAAATTTTGGCTCGACCATAAAAACCTATGGGAACAAAGCTTGGAACAGCAGCACAGGGCAGGCCCTTA
GAAATAAGCTTAAGGAGCAGAACTTCCAACAAAAGGTGGTCGATGGTATCGCCTCTGGTATTAACGGCGTAGTGGAT
CTAGCCAACCAGGCTGTGCAGAAACAGATAAACAGCCGCCTGGACCCGCCGCCCGCAACTCCTGGTGAAATGGAAGT
GGAGGAAGAGCTTCCTCCGCTGGAGAAGCGGGGCGACAAGCGACCGCGTCCCGAGCTGGAACAGACGTTGGTGACGC
GCGCAGACGAGCCCCCTTCATACGAGGAGGCAGTAAAGCTCGGAATGCCCACTACCAGGCCTGTAGCTCACATGGCT
ACCGGGGTGATGAAACCTTCTCAGTCGCATCGGCCTGCCACCTTGGACTTGCCTCCTCCCCCTGCTTCTGCGGCGCC
TATTCCCAAACCTGTCGCTACCAGAAAGCCCACCGCCGTACAGCCCGTCGCCGTAGCCAGACCGCGTCCTGGGGCAC
ACCGCGCCCGAAAGCAAACTGGCAGAGTACTCTGAACAGCATCGTGGGTCTGGGCGTGCAGAGTGTAAAGCGCCGTC
GCTGCTATTAATTAAATATGGAGTAGCGCTTAACTTGCTTGTCTGTGTGTATGTATCATCACCATGCCGCCGCAGCA
GAGGAGAAAGGAAGAGGTCGCGCGCCGAGGCTGAGTTGCTTTCAAGATGGCCACCCCATCGATGATGCCCCAATGGG
CATACATGCACATCGCCGGACAGGATGCTTCGGAGTACCTGAGTCCGGGTCTGGTGCAGTTCGCCCGTGCAACAGAC
ACCTACTTCAGTATGGGGAACAAGTTTAGAAACCCCACAGTGGCGCCCACCCACGATGTGACCACCGACCGTAGCCA
GCGACTAATGCTGCGCTTCGTGCCCGTTGACCGGGAAGACAATACCTACTCTTACAAAGTTCGCTACACGCTGGCTG
TAGGGGACAACAGAGTACTGGATATGGCCAGCACGTTCTTTGACATCCGCGGCGTGCTGGACCGGGGCCCTAGCTTC
AAACCCTACTCCGGGACCGCCTACAACAGCCTGGCTCCCAAGGGAGCGCCCAACACCTGCCAGTGGAAGGATTCTGA
CAGCAAAATGCATACCTTTGGGGTAGCTGCCATGCCCGGTGTTACTGGGAAAAAGATAGAAGCTGATGGGCTGCCTA
TTGGAATAGATTCAACTTCTGGAACTGACACAGTAATTTATGCTGATAAAACTTTCCAACCAGAACCACAAGTTGGA
AATGCCAGTTGGGTTGACGCCAATGGTACAGAGGAAAAATATGGAGGCAGAGCTCTGAAGGACACTACAAAGATGAA
ACCCTGCTATGGTTCTTTCGCCAAGCCTACCAACAAAGAAGGTGGTCAGGCTAACTTAAAAGATTCAGAAACCGCCG
CCACCACTCCTAACTATGATATAGATCTGGCTTTCTTTGACAACAAAAATATTGCTGCTAACTACGATCCAGATATT
GTAATGTACACAGAAAATGTTGACTTGCAGACTCCAGATACTCATATTGTATACAAACCTGGAACAGAGGACACCAG
CTCTGAATCCAATTTGGGTCAGCAAGCCATGCCTAACAGACCCAACTACATTGGCTTCAGAGACAATTTTATTGGGC
TCATGTACTACAACAGCACTGGCAATATGGGGGTGCTGGCCGGTCAGGCCTCTCAGCTGAATGCTGTGGTTGACTTG
CAAGACAGAAACACTGAACTGTCCTACCAGCTCTTGCTTGACTCTCTGGGTGACAGAACCCGGTATTTCAGTATGTG
GAATCAGGCGGTGGACAGCTATGATCCTGATGTGCGTATTATTGAAAACCATGGTGTGGAGGACGAATTGCCAAACT
ATTGCTTTCCGTTGAATGGTGTGGGATTTACAGACACTTACCAAGGTGTTAAAGTTAAAACAGATGCAGTTGCTGGA
ACCAGTGGAACACAGTGGGACAAAGATGACACCACAGTTAGTACTGCTAATGAAATCCATGGAGGCAATCCTTTTGC
CATGGAAATCAACATCCAAGCCAATCTATGGCGAAGTTTCCTTTATTCCAATGTGGCTTTGTATCTCCCAGACTCGT
ATAAATACACCCCGTCCAATGTCACTCTCCCAGAAAACAAAAACACCTACGACTACATGAACGGGCGGGTGGTGCCG
CCATCTCTAGTAGACACCTATGTGAACATTGGTGCCAGGTGGTCTCTGGATGCCATGGACAATGTCAACCCATTCAA
CCACCACCGTAACGCTGGCTTGCGTTACCGATCCATGCTTCTGGGTAACGGACGTTATGTGCCTTTCCACATACAAG
TGCCTCAAAAATTCTTCGCTGTTAAAAACCTGCTGCTTCTCCCAGGCTCCTACACTTATGAGTGGAACTTTAGGAAG
GATGTAAACATGGTTCTACAGAGTTCCCTTGGTAACGACCTACGGGTAGATGGCGCCAGCATCAGTTTCACGAGCAT
CAATCTTTATGCTACTTTTTTCCCCATGGCTCACAACACCGCTTCCACCCTTGAAGCCATGCTGCGGAATGACACCA
ATGATCAGTCATTCAACGACTACCTATCTGCAGCTAACATGCTCTACCCCATACCTGCCAACGCAACCAATATTCCC
ATTTCCATTCCTTCTCGCAACTGGGCGGCTTTCAGAGGCTGGTCATTTACCAGACTGAAAACCAAAGAAACTCCCTC
TTTGGGGTCTGGATTTGACCCATACTTTGTCTATTCCGGTTCTATTCCCTACCTGGATGGTACCTTCTATCTAAATC
ACACTTTTAAGAAGGTTTCCATCATGTTTGACTCTTCAGTGAGCTGGCCTGGAAATGACAGGTTACTATCTCCTAAC
GAATTTGAAATAAAGCGCACTGTGGATGGCGAAGGCTACAACGTAGCCCAATGCAACATGACCAAAGACTGGTTCTT
GGTACAGATGCTCGCCAACTACAACATTGGCTACCAGGGCTTTTACATCCCTGAGGGATACAAGGATCGCATGTACT
CCTTTTTCAGAAACTTCCAGCCTATGAGCAGGCAGGTGGTTGATGAGGTTAATTACACTGACTACAAAGCCGTCACC
TTACCATATCAACACAACAACTCTGGCTTTGTAGGATACCTTGCGCCTACTATGAGACAAGGGGAACCTTACCCAGC
CAATTATCCATACCCGCTCATCGGAACTACTGCCGTTAAGAGTGTTACCCAAAAAAAGTTCCTGTGCGACAGGACCA
TGTGGCGCATACCGTTCTCCAGCAACTTCATGTCCATGGGGGCCCTTACAGACCTGGGACAAAACCTGCTCTATGCC
AACTCGGCCCATGCACTGGACATGACTTTTGAGGTGGATCCCATGGATGAGCCCACCCTGCTTTATCTTCTTTTCGA
AGTCTTCGACGTGGTCAGAGTGCACCAGCCACACCGCGGCGTCATCGAGGCCGTCTACCTGCGCACACCGTTCTCGG
CCGGCAACGCCACCACATAAGAAGCCTCTTGCTTCTTGCAAGCAGCAGCTGCAGCCATGTCATGCGGGTCCGGAAAC
GGCTCCAGCGAGCAAGAGCTCAAAGCCATCGTCCGAGACCTGGGTTGCGGTCCCTATTTCCTGGGAACCTTTGACAA
GCGTTTCCCGGGGTTCATGGCCCCCGACAAGCTCGCCTGCGCCATAGTCAACACTGCCGGACGCGAGACGGGGGGAG
AGCACTGGCTGGCTTTTGGTTGGAACCCGCGCTCCAACACCTGCTACCTTTTTGATCCTTTTGGGTTCTCGGATGAG
CGACTCAAACAGATTTACCAGTTTGAGTACGAGGGGCTCCTGCGCCGCAGTGCCCTTGCTACCAAAGACCGCTGCAT
CACCCTGGAAAAGTCCACCCAGAGCGTGCAGGGCCCACGCTCAGCCGCCTGTGGACTTTTTTGCTGTATGTTCCTTC
ATGCCTTTGTGCACTGGCCCGACCGTCCCATGAACGGAAACCCCACCATGAAGTTGCTGACTGGGGTGCCCAACAGC
ATGCTCCAATCTCCCCAAGTCCAGCCCACCCTGCGCCGCAACCAGGAGGCACTATACCGCTTCCTAAACACCCACTC
ATCTTACTTTCGTTCTCACCGCGCACGCATCGAAAGGGCCACCGCGTTTGACCGTATGGATATGCAATAAGTCATGT
AAAACCGTGTTCAATAAAAAGCACTTTATTTTTACATGCACTAAGGCTCTGGTTTTTTGCTCATTCGTTTTCATCAT
TCACTCAGAAATCAAATGGGTTCTGGCGGGAGTCATAGTGGCCCGCGGGCAGGGATACGTTGCGGAACTGTAACCTG
TTCTGCCACTTGAACTCGGGGATCACCAGCTTGGGAACTGGAATCTCGGGAAAGGTGTCTTGCCACAACTTTCTGGT
CAGTTGCAGGGCGCCAAGCAGGTCAGGAGCAGAGATCTTGAAATCACAGTTGGGGCCGGCATTCTGGACACGGGAGT
TGCGGTACACTGGGTTGCAACACTGGAACACCATCAAGGCTGGGTGTCTCACGCTTGCCAGCACGGTCGGGTCACTG
ATGGTAGTCACATCCAAGTCTTCAGCATTGGCCATCCCAAAGGGGGTCATCTTACAGGTCTGCCTGCCCATCACGGG
AGCGCAGCCTGGCTTGTGGTTGCAATCGCAATGAATGGGAATCAGCATCATCCTGGCTTGGTCGGGGGTTATCCCTG
GATATACGGCCTTCATGAAGGCTTCGTACTGCTTGAAAGCTTCCTGAGCCTTACTTCCCTCGGTGTAGAACATTCCA
CAGGACTTGCTGGAAAATTGGTTAGTAGCACAGTTGGCATCATTTACACAGCAGCGGGCATCGTTGTTGGCCAACTG
AACCACATTTCTGCCCCAGCGGTTCTGGGTGATCTTGGCTCTGTCTGGGTTCTCCTTCATAGCGCGCTGCCCGTTCT
CGCTCGCCACATCCATCTCGATAATGTGGTCCTTCTGGATCATGATAGTGCCATGCAGGCATTTCACCTTGCCTTCG
TAATCGGTGCATCCATGAGCCCACAGAGCGCACCCGGTGCACTCCCAATTATTGTGGGCGATCTCAGAATAAGAATG
CACCAATCCCTGCATGAATCTTCCCATCATCGCTGTCAGGGTCTTCATGCTACTAAATGTCAGCGGAATGCCACGGT
GCTCCTCGTTCACATACTGGTGGCAGATACGCTTGTACTGCTCGTGCTGCTCTGGCATCAGCTTGAAAGAGGTTCTC
AGGTCATTATCCAGCCTATACCTCTCCATTAGCACAGCCATCACTTCCATGCCCTTCTCCCAGGCAGATACCAGGGG
CAAGCTCAAAGGATTCCTAACAGCAATAGAAGTAGCTCCTTTAGCTATAGGGTCATTCTTGTCGATCTTCTCAACAC
TTCTCTTGCCATCCTTCTCAATGATGCGCACCGGGGGGTAGCTGAAGCCCACGGCCACCAACTGAGCCTGTTCTCTT
TCTTCTTCGCTGTCCTGGCTGATGTCTTGCAGAGGGACATGCTTGGTCTTCCTGGGCTTCTTCTTGGGAGGGATCGG
GGGAGGACTGTTGCTCCGTTCCGGAGACAGGGATGACCGCGAAGTTTCGCTTACCAGTACCACCTGGCTCTCGATAG
AAGAATCGGACCCCACGCGACGGTAGGTGTTCCTCTTCGGGGGCAGAGGTGGAGGCGACTGAGATGGGCTGCGGTCT
GGCCTTGGAGGCGGATGGCTGGCAGAGCCCATTCCGCGTTCGGGGGTGTGCTCCCGTTGGCGGTCGCTTGACTGATT
TCCTCCGCGGCTGGCCATTGTGTTCTCCTAGGCAGAGAAACAACAGACATGGAAACTCAGCCATCACTGCCAACATC
GCTGCAAGCGCCATCACACCTCGCCCCCAGCAGCGACGAGGAGGAGAGCTTAACCACCCCACCACCCAGTCCCGCTA
CCACCACCTCTACCCTCGATGATGAGGAGGAGGTCGACGCAGCCCAGGAGATGCAGGCGCAGGATAATGTGAAAGCG
GAAGAGATTGAGGCAGATGTCGAGCAGGACCCGGGCTATGTGACACCGGCGGAGCACGAGGAGGAGCTGAAACGTTT
TCTAGACAGAGAGGATGACGACCGCCCAGAGCATCAAGCAGATGGCGATCACCAGGAGGCTGGCCTCGGGGATCATG
TTGCCGACTACCTCTCCGGGCGTGGGGGGGAGGACGTGCTCCTCAAACATCTAGCAAGGCAGTCGCTCATAGTTAAA
GACGCACTACTCAACCTCACCGAAGTGCCCATCAGTGTGGAAGAGCTTAGCCGCGCCTACGAGCTGAACCTCTTTTC
GCCTCAGATACCCCCCAAGCGGCAGCCAAACGGCACCTGCGAGGCCAACCCTCGACTCAACTTCTATCCAGCTTTTA
CTGTCCCCGAAGTGCTGGCCACCTACCACATCTTTTTTAAGAACCAAAAGATTCCAGTCTCCTGCCGCGCCAACCGC
ACCCGCGCAGATGCCCTTCTCAACTTGGGTCCGGGAGCTCGCTTACCTGATATAGCTTCCTTGGAAGAGGTTCCAAA
GATCTTTGAGGGTCTGGGAAGTGATGAGACTCGGGCCGCAAATGCTCTGCAACAGGGAGAGAATGGCATGGATGAAC
ATCACAGCGCTCTAGTGGAACTGGAGGGTGACAATGCCCGGCTTGCAGTGCTCAAGCGCAGTATCGTGGTCACCCAT
TTTGCCTACCCCGCTGTTAACCTGCCGCCCAAAGTCATGAGCGCTGTCATGGACCATCTGCTCATCAAACGAGCAAG
TCCACTTTCAGAAAACCAGAACATGCAGGATCCAGACGCCTCGGACGAGGGCAAGCCGGTAGTCAGTGACGAGCAGC
TATCTCGCTGGCTGGGTACCAACTCCCCCCGAGATTTGGAAGAAAGACGCAAGCTTATGATGGCTGTAGTGCTAGTA
ACTGTTGAGTTGGAGTGTCTGCGCCGCTTTTTTACCGACCCCGAGACCCTGCGCAAGCTAGAGGAGAACCTGCACTA
CACCTTCAGACATGGCTTCGTGCGGCAGGCATGCAAGATCTCCAACGTGGAGCTCACCAACCTGGTTTCATACATGG
GCATTTTGCATGAGAACCGGCTAGGGCAGAGCGTTCTGCACACCACCCTGAAGGGGGAGGCCCGCCGCGACTACATC
CGAGACTGTGTCTACCTCTACCTCTGCCATACCTGGCAGACTGGTATGGGTGTGTGGCAACAGTGTTTGGAAGAGCA
GAACCTTAAAGAGCTGGACAAGCTCTTGCAGAGATCCCTCAAAGCCCTGTGGACAGGTTTTGACGAGCGCACCGTCG
CCTCGGACTTGGCGGACATCATCTTCCCCGAGCGTCTTACGGTTACTCTGCGAAACGGCCTGCCAGACTTCATGAGC
CAGAGCATGCTTAACAACTTTCGCTCTTTCATCCTGGAACGCTCCGGTATCCTGCCTGCCACCTGCTGTGCGCTGCC
CTCCGACTTTGTGCCTCTCACCTACCGCGAGTGCCCACCGCCGCTATGGAGCCACTGCTACCTATTCCGCCTGGCCA
ACTACCTCTCCTACCACTCGGATGTGATAGAGGATGTGAGCGGAGACGGCCTGCTGGAATGCCACTGCCGATGCAAT
TTATGCACACCCCACCGCTCCCTCGCCTGCAACCCCCAGTTGCTAAGCGAGACCCAGATCATCGGCACCTTCGAGTT
GCAGGGTCCCAACAGTGAAGGCGAGGGGTCTTCTCCGGGGCAGAGTCTGAAACTGACACCGGGGCTGTGGACCTCCG
CCTACCTGCGCAAGTTTCATCCCGAGGACTATCATCCCTATGAGATCAGGTTCTATGAGGACCAGTCACATCCTCCC
AAAGTCGAGCTCTCAGCCTGCGTCATCACCCAGGGGGCAATTCTGGCCCAATTGCAAGCCATCCAAAAATCCCGCCA
AGAATTTCTGCTGAAAAAGGGAAGCGGGGTCTACCTTGACCCCCAGACCGGTGAGGAGCTCAACACAAGGTTCCCCC
AGGATGTCCCATCGCCGAGGAAGCAAGAAGCTGAAGGTGCAGCTGTCACCCCCAGAGGATATGGAGGAAGACTGGGA
CAGTCAGGCAGAGGAGGAGATGGAAGATTGGGACAGCCAGGCAGAGGAGGTGGACAGCCTGGAGGAAGACAGTTTGG
AGGAGGAAGACGAGGAGGCAGAGGAGGTGGAAGAAGCAACCGCCGCCAAACAGTTGTCATCGGCGGCGGAGACAAGC
AAGTCCCCAGACAGCAGCACGGCTACCATCTCCGCTCCGGGTCGGGGGGCCCAGCGGCGGCCCAACAGTAGATGGGA
CGAGACCGGGCGATTCCCAAACCCGACCACCGCTTCCAAGACCGGTAAGAAGGAGCGACAGGGATACAAGTCCTGGC
GTGGACATAAAAACGCTATCATCTCCTGCTTGCATGAATGCGGGGGCAACATATCCTTCACCCGGCGATACCTGCTT
TTCCACCACGGTGTGAACTTCCCCCGCAATATCTTGCATTACTACCGTCACCTCCACAGCCCCTACTGCAGTCAGCA
AGTCCCGGCAACCCCGACAGAAAAAGACAGCAGCGACAACGGTGACCAGAAAACCAGCAGTTAGAAAATCCACAACA
AGTGCAGCAGGAGGAGGACTGAGGATCACAGCGAACGAGCCAGCGCAGACCAGAGAGCTGAGGAACCGGATCTTTCC
AACCCTCTATGCCATCTTCCAGCAGAGTCGGGGGCAAGAGCAGGAATTAAAAGTAAAAAACCGATCTCTGCGCTCGC
TCACCAGAAGTTGTTTGTATCACAAGAGCGAAGACCAACTTCAGCGCACTCTCGAGGACGCCGAGGCTCTCTTCAAC
AAGTACTGCGCGCTGACTCTTAAAGAGTAGCCCTTGCCCGCGCTCATTCGAAAACGGCGGGAATCACGTCACCCTTG
GCAGCTGTCCTTTGCCCTCGTCATGAGTAAAGAGATTCCCACGCCTTACATGTGGAGCTATCAGCCCCAAATGGGGT
TGGCAGCAGGTGCTTCCCAGGACTACTCCACCCGCATGAATTGGCTTAGCGCCGGGCCCTCAATGATATCACGGGTT
AATGATATACGAGCTTATCGAAACCAGTTACTCCTAGAACAGTCAGCTCTCACCACCACACCCCGCCAACACCTTAA
TCCCCGAAATTGGCCCGCCGCCCTGGTGTACCAGGAAAATCCCGCTCCCACCACCGTACTACTTCCTCGAGACGCCC
AGGCCGAAGTTCAGATGACTAACGCAGGTGTACAGCTGGCGGGCGGTTCCGCCCTATGTCGTCACCGACCTCAACAG
AGTATAAAACGCCTGGTGATCAGAGGCCGAGGTATCCAGCTCAACGACGAGTCGGTTAGCTCTTCGCTTGGTCTGCG
ACCAGACGGAGTCTTCCAGATCGCCGGCTGTGGGAGATCTTCCTTCACTCCTCGTCAGGCTGTGCTGACTTTGGAGA
GTTCGTCCTCGCAGCCCCGCTCGGGCGGCATCGGAACTCTCCAGTTTGTGGAGGAGTTTACTCCCTCTGTCTACTTC
AACCCCTTCTCCGGCTCTCCTGGCCAGTACCCGGACGAGTTCATACCGAACTTCGACGCAATCAGCGAGTCAGTGGA
TGGCTATGATTGATGTCTAATGGTGGCGCGGCTGAGCTAGCTCGACTGCGACACCTAGACCACTGCCGCCGCTTTCG
CTGTTTCGCCCGGGAACTCACCGAGTTCATCTACTTCGAACTCTCCGAGGAGCACCCTCAGGGTCCGGCCCACGGAG
TGCGGATTACCATCGAAGGGGGAATAGACTCTCGCCTGCATCGCATCTTCTCCCAGCGGCCCGTGCTGATTGAGCGC
GACCAGGGAAATACAACCATCTCCATCTACTGCATTTGTAACCACCCCGGATTGCATGAAAGCCTTTGCTGTCTTGT
TTGTGCTGAGTTTAATAAAAACTGAGTTAAGACCCTCCTACGGACTACCGCTTCTTCAATCAGGACTTTACAACACC
AACCAGATCTTCCAGAAGACCCAGACCCTTCCTCCTCTGATCCAGGACTCTAACTCTACCTTACCAGCACCCTCCAC
TACTAACCTTCCCGAAACTAACAAGCTTGGATCTCATCTGCAACACCGCCTTTCACGAAGCCTTCTTTCTGCCAATA
CTACCACTCCCAAAACCGGAGGTGAGCTCCGCGGTCTTCCTACTGACGACCCCTGGGTGGTAGCGGGTTTTGTAACG
TTAGGAGTAGTTGCGGGTGGGCTTGTGCTAATCCTTTGCTACCTATACACACCTTGCTGTGCATATTTAGTCATATT
GTGCTGTTGGTTTAAGAAATGGGGGCCATACTAGTCGTGCTTGCTTTACTTTCGCTTTTGGGTCTGGGCTCTGCTAA
TCTCAATCCTCTTGATCACGATCCATGTCTAGACTTCGACCCAGAAAACTGCACACTTACTTTTGCACCCGACACAA
GCCGTCTCTGTGGAGTTCTTATTAAGTGCGGATGGGACTGCAGGTCCGTTGAAATTACACATAATAACAAAACATGG
AACAATACCTTATCCACCACATGGGAGCCAGGAGTTCCCGAGTGGTATACTGTCTCTGTCCGAGGTCCTGACGGTTC
CATTCGCATTAGTAACAACACTTTCATTTTTTCTGAAATGTGCGATCTGGCCATGTTTATGAGCAAACAGTATGACC
TATGGCCTCCTAGCAAAGAGAACATTGTGGCATTTTCCATTGCTTATTGCTTGGTAACATGCATCATCACTGCTATC
ATTTGTGTGTGCATACACTTGCTTATAGTTATTCGCCCTAGACAAAGCAATGAGGAAAAAGAGAAAATGCCTTAACC
TTTTTCCTCATACCTTTTCTTTACAGCATGGCTTCTGTTACAGCTCTAATTATTGCCAGCATTGTCACTGTCGCTCA
CGGGCAAACAATTGTCCATATTACCTTAGGACATAATCACACTCTTGTAGGGCCCCCAATTACTTCAGAGGTTATTT
GGACCAAACTTGGAAGTGTTGATTATTTTGATATAATTTGCAACAAAACTAAACCAATATTTGTAATCTGCAACAGA
CAAAATCTCACGTTAATTAATGTCAGCAAAATTTATAACGGTTACTATTATGGTTATGATAGATCCAGTAGTCAATA
TAAAAATTACTTAGTTCGCATAACTCAACCCAAATCAACAGTGCCAACTATGACAATAATTAAAATGGCTAATAAAG
CATTAGAAAATTTTACATTACCAACAACGCCCAATGAAAAAAACATTCCAAATTCAATGATTGCAATTATTGCGGCG
GTGGCATTGGGAATGGCACTAATAATAATATGCATGTTCCTATATGCTTGTTGCTATAAAAAGTTTAAACATAAACA
GGATCCACTACTAAATTTTAACATTTAATTTTTTATACAGATGATTTCCACTACAATTTTTATCATTACTAGCCTTG
CAGCTGTAACTTATGGCCGTTCACACCTAACTGTACCTGTTGGCTCAACATGTACACTACAAGGACCCCAAGAAGGC
CATGTCACTTGGTGGAGAATATATGATAATGGAGGGTTCGCTAGACCATGTGATCAGCCTGGTACAAAATTTTCATG
CAACGGAAGAGACTTGACCATTATTAACATAACATTAAATGAGCAAGGCTTCTATTATGGAACCAACTATAAAAATA
GTTTAGATTACAACATTATTGTAGTGCCAGCCACCACTTCTGCTCCCCGCAAATCCACTTTCTCTAGCAGCAGTGCC
AAAGCAAGCACAATTCCTAAAACAGCTTCTGCTATGTTAAAGCTTCGAAAAATCGCTTTAAGTAATTCCACAGCCGC
TCCCAATACAATTCCTAAATCAACAATTGGCATCATTACTGCCGTGGTAGTGGGATTAATAATTATATTTTTGTGCA
TAATGTACTACGCCTGCTGCTATAGAAAACATGAACAAAAAGGTGATGCATTACTAAATTTTGATATTTAATTTTTT
ATAGAATTATGATATTGTTTCAATCAAATACCACTAACACTATCAATGTGCAGACTACTTTAAATCATGACATGGAA
AACCACACTACCTCCTATGCATACACAAACATTCAGCCTAAATACGCTATGCAACTAAGAAATCACCATACTAATTG
TAATTGGAATTCTTATACTATCTGTTATTCTTTATTTTATATTCTGCCGTCAAATACCCAATGTTCATAGAAATTCT
AAAAGACGACCCATCTATTCTCCTATGATTAGTCGTCCCCATATGGCTCTGAATGAAATCTAAGATCTTTTTTTTTC
TCTTACAGTATGGTGAACATCAATCATGATTCCTAGAAATTTCTTCTTCACCATACTCATCTGTGCTTTCAATGTCT
GTGCTACTTTCACAGCAGTAGCCACTGCAAGCCCAGACTGTATAGGACCATTTGCTTCCTATGCACTTTTTGCCTTC
GTTACTTGCATCTGCGTGTGTAGCATAGTCTGCCTGGTTATTAATTTTTTCCAACTGGTAGACTGGATCTTTGTGCG
AATTGCCTACCTACGTCACCATCCCGAATACCGCAATCAAAATGTTGCGGCACTTTTTAGGCTTATTTAAAACCATG
CAGGCTATGCTGCCAGTCATTTTAATTCTGCTCCTACCCTGCATTGCCCTAGCTTCCACCGCCACTCGCGCTACACC
TGAACAACTTAGAAAATGCAAATTTCAACAACCATGGTCATTTCTTGATTGCTACCATGAAAAATCTGATTTTCCCA
CATACTGGATAGTGATTGTTGGAATAATTAACATACTTTCATGTACCTTTTTCTCAATCACAATATACCCCACATTT
AATTTTGGGTGGAATTCTCCCAATGCACTGGGTTACCCACAAGAACCACATGAACATATCCCACTACAACACATACA
ACAACCACTAGCACTGGTAGAGTATGAAAATGAGCCACAACCTTCACTGCCTCCTGCCATTAGTTACTTCAACCTAA
CCGGCGGAGATGACTGAAATACTCACCACCTCCAATTCCGCCGAGGATCTGCTTGATATGGACGGCCGCGCCTCAGA
ACAGCGACTCGCCCAACTACGCATACGCCAGCAGCAGGAACGTGCCGCCAAGGAGCTCAGGGATGCTATTGAAATTC
ACCAATGCAAAAAAGGCATATTTTGTTTGGTAAAACAAGCCAAGATATCCTACGAGATTACCAATACTGACCATCGC
CTCTCATACGAGCTCGGACCGCAGCGACAAAAATTCACTTGTATGGTGGGAATCAACCCCATAATCATCACCCAGCA
AGCTGGAGATACCAAGGGTTGCATCCACTGTTCCTGCAGTTCCGCCGAGTGCATCTACACCCTGCTGAAGACCCTCT
GCGGCCTTCGAGACCTCCTACCCATGAACTAATCAACCCAGCCCCTCACTTACCAATTACATAAAGCCAATTAATAA
AAACACTTACTTGAAATCAGAAATAAGGTTTCTGTCTACGTTGTTTCCAAGCAGCACCTCACTTCCTTCTTCCCAAC
TCTGGTACTCTAAGCCTCGGCGGGTGGCATACTTCCTCCACACTTTGAAAGGGATGTCAAATTTTAGTTCCTCTTCT
TTGCCCACAATCTTCATTTCTTTATCCCCAGATGGCCAAACGAGCTCGGCTAAGCAGCTCCTTCAATCCGGTCTACC
CCTATGAAGATGAAAGCAGCTCACAACACCCCTTTATAAACCCTGGTTTCATTTCCTCAAATGGTTTTGCACAAAGC
CCAGATGGAGTTCTAACTCTTAAATGTGTTAATCCACTCACTACCGCCAGCGGACCCCTCCAACTTAAAGTTGGAAG
CAGTCTTACAGTAGATACTATCGATGGGTCTTTGGAGGAAAATATAACTGCCGCAGCGCCACTCACTAAAACTAACC
ACTCCATAGGTTTATTAATAGGATCTGGCTTGCAAACAAAGGATGATAAACTTTGTTTATCGCTGGGAGATGGGTTG
GTAACAAAGGATGATAAACTATGTTTATCGCTGGGAGATGGGTTAATAACAAAAAATGATGTACTATGTGCCAAACT
AGGACATGGCCTTGTGTTTGACTCTTCCAATGCTATCACCATAGAAAACAACACCTTGTGGACAGGCGCAAAACCAA
GCGCCAACTGTGTAATTAAAGAGGGAGAAGATTCCCCAGACTGTAAGCTCACTTTAGTTCTAGTGAAGAATGGAGGA
CTGATAAATGGATACATAACATTAATGGGAGCCTCAGAATATACTAACACCTTGTTTAAAAACAATCAAGTTACAAT
CGATGTAAACCTCGCATTTGATAATACTGGCCAAATTATTACTTACCTATCATCCCTTAAAAGTAACCTGAACTTTA
AAGACAACCAAAACATGGCTACTGGAACCATAACCAGTGCCAAAGGCTTCATGCCCAGCACCACCGCCTATCCATTT
ATAACATACGCCACTGAGACCCTAAATGAAGATTACATTTATGGAGAGTGTTACTACAAATCTACCAATGGAACTCT
CTTTCCACTAAAAGTTACTGTCACACTAAACAGACGTATGTTAGCTTCTGGAATGGCCTATGCTATGAATTTTTCAT
GGTCTCTAAATGCAGAGGAAGCCCCGGAAACTACCGAAGTCACTCTCATTACCTCCCCCTTCTTTTTTTCTTATATC
AGAGAAGATGACTGACAACAAAAAAAATAAAGATCAACTTTTTTATTGAAAATCAGTTTACAAGATTCGAGTAGTTA
TTTTGCCCCCCTCTTCCCATTTTATAGAATACACAATTCTCTCCCCACGCACAGCTTTGAACATTTGAATTCCATTA
GAGATAGACATAGTTTTAGATTCCACATTCCACACAGTTTCAGAGCGGGCCAATCTTGGATCAGTGATAGATATAAA
TCCATCGGAACAGTCTTTCAAGGTGGTTTCACAGTCCAACTGCTGCGGCTGCGGCTCCGGGGTTTGGATTAGGGTCA
TCTGGAAGAAGAACGATGGGAGTCATAATCCGAGAACGGGATCGGGCGGTTGTGTCTTAAACCTCGAAGCAATCGCT
GTCTGCGCCGCTCCGTGCGACTGCTGCTGATGGGATCAGGATCCACAGTCTCTCGAAGCATAATTTTAATAGCCCTC
AACATTAACATCCTGGTGCGATGGGCACAACAACGCATTCTAATTTCGCTTAGCTCACTGCAGTAGGTACAACACAT
TACCACAATGTTGTTTAACAGGCCATAATTAAAGGTGCTCCAGCCAAAACTCATCTCAGGGATAATCATACCCGCGT
GACCATCGTACCAAATCTTAATGTAAATTAGATGACGCCCCCTCCAGAACACACTGCCCACATACATAATTTCCTTG
GGCATATGCATGTTCACAATTTCTCTGTACCATGGACAGCGCTGGTTAATCATACAGCCCCTAATAACCTTCCGGAA
CCACATAGCTAGCACTGCTCCCCCAGCAATACATTGAAGAGAACCCGGCTGTTTACAGTGACAATGGAGAACCCACT
TCTCTCGCCCATGGATCACTTGAGAATTAAATATATCTATAGTGGCACAACACAAACATAAATGCATGCATCTTTTC
ATAACCCTCAACTCTTCGGGGGTTAAAAACATATCCCAGGGAATAGGAAGCTCTTGCAAAACAGTAAAGCTGGCAGA
ACAAGGAAGACCACGAACACAACTTACACTATGCATAGTCATAGTATCACAATCTGGCAACAGCGGGTGGTCTTCAG
TCATAGAAGCTCGGGTTTCATTTTCCTCACATCGTGGTAACTGGGCTCTGGTGTAAGGGTGATGTCTGGCGCATGAT
GTCGAGCGTGCGCGCAACCTTGTCATAATGGAGTTGCTTCCTGACATTCTCGTATTTTGTATAGCAAAATGCGGCCC
TGGCACAACACACTTTTCTTCGTCTTCTATCCTGCCGCTTAGTGTGTTCCGTCTGATAATTCAAGTACAGCCACACT
CTTAAGTTGGTCAAAAGAATGCTGGCTTCAGTTGTAATCAAAACTCCATCATATTTAATTGTTCTAAGGAAATCATC
CACGGTAGCATATGCAAATCCCAACCAAGCAATGCAACTGGATTGCGTTTCAAGCAGCAGAGGAGAGGGAAGAGACG
GAAGAATCATGTTAATTTTTATTCCAAACGATCTCGCAGTACTTCAAATTGTAGATCGCGCAGATGGCATCTATCGC
CCCCACTGTGTTGGTGAAAAAGCACAGCTAAATCAAAAGAAATGCGATTTTCAAGGTGCTCAACGGTGGCTTCCAAC
AAAGCCTCCACGCGCACATCCAAAAACAAAAGAATACCAAAAGAAGGAGCATTTTCTAACTCCTCAAACATCATATT
ACATTCCTGCACCATTCCCAGATAATTTTCAGCTTTCCAGCCTTGAATTATTCGTGTCAGTTCTTGTGGTAAATCCA
AACCACACATTACAAACAGGTCCCGGAGGGCGCCCTCCACCACCATTCTTAAACACACCCTCATAATGACAAAATAT
CTTGCTCCTGTGTCACCTGTAGCAAATTAAGAATGGCATCATCAATTGACATGCCCTTGGCTCTAAGTTCTTCTCTA
AGTTCTAGTTGTAAATACTCTCTCATATTATCACCAAACTGCTTAGCCAGAAGCCCCCCGGGAACAATAGCAGGGGA
CGCTACAGTGCAGTACAAGCGCAGACCTCCCCAATTGGCTCCAGCAAAAACAAGATTAGAATAAGCATACTGGGAAC
CACCAGTAATATCATCAAAGTTGCTGGAAATATAATCAGGCAGAGTTTCTTGTAAAAATTGAATAAAAGAAAAATTT
TCCAAAGAAACATTCAAAACCTCTGGGATGCAAATGCAATAGGTTACCGCGCTGCGCTCCAACATTGTTAGTTTTGA
ATTAGTCTGTAAAATAAAAGAAACAAGCGTCATATCATAGTAGCCTGTCGAACAGGTGGATAAATCAGTCTTTCCAT
CACAAGACAAGCCACAGGGTCTCCAGCTTGACCCTCGTAAAACCTGTCATCGTGATTAAACAACAGCACCGAAAGTT
CCTCGCGGTGGCCAGCATGAATAATTCTTGATGAAGCATATAATCCAGACATGTTAGCATCAGTTAAAGAGAAAAAA
CAGCCAACATAGCCTCTGGGTATAATTATGCTTAATCTTAAGTATAGCAAAGCCACCCCTCGCGGATACAAAGTAAA
AGGCACAGGAGAATAAAAAATATAATTATTTCTCTGCTGCTGTTCAGGCAACGTCGCCCCCGGTCCCTCTAAATACA
CATACAAAGCCTCATCAGCCATGGCTTACCAGACAAAGTACAGCGGGCGCACAAAGCACAAGCTCTAAAGAAGCTCT
AAAGACACTCTCCAACCTCTCCACAATATATACACAAGCCCTAAACTGACGTAATGGGAGTAAAGTGTAAAAAATCC
CGCCAAGCCCAACACACACCCCGAAACTGCGTCAGCAGGGAAAAGTACAGTTTCACTTCCGCAAACCCAACAAGCGT
AACTTCCTCTTTCTCACGGTACGTCACATCCGATTAACTTGCAACGTCATTTTCCCACGGCCGCACCGCCCCTTTTA
GCCGTTCACCCCGCAGCCAATCACCACACAGCGCGCACTTTTTTAAATTACCTCATTTACATGTTGGCACCATTCCA
TCTATAAGGTATATTATTGATAATG

GenBank Accession No. AAW33461 (SEQ ID NO: 226)

MAKRARLSSSFNPVYPYEDESSSQHPFINPGFISSNGFAQSPDGVLTLKCVNPLTTASGPLQLKVGSSLTVDTIDGS
LEENITAAAPLTKINHSIGLLIGSGLQTKDDKLCLSLGDGLVTKDDKLCLSLGDGLITKNDVLCAKLGHGLVFDSSN
AITIENNTLWTGAKPSANCVIKEGEDSPDCKLTLVLVKNGGLINGYITLMGASEYTNTLFKNNQVTIDVNLAFDNTG
QIITYLSSLKSNLNFKDNONMATGTITSAKGFMPSTTAYPFITYATETLNEDYIYGECYYKSTNGTLFPLKVTVTLN
RRMLASGMAYAMNFSWSLNAEEAPETTEVTLITSPFFFSYIREDD

GenBank Accession No. AAW33439 (SEQ ID NO: 227)

MRRRAVLGGAVVYPEGPPPSYESVMQQQAAMIQPPLEAPFVPPRYLAPTEGRNSIRYSELSPQYDTTKLYLVDNKSA
DIASLNYQNDHSNFLTTVVONNDFTPTEASTQTINFDERSRWGGQLKTIMHTNMPNVNEYMFSNKFKARVMVSRKAP
EGVTVNDHKDDILKYEWFEFTLPEGNFSATMTIDLMNNAIIDNYLKIGRONGVLESDIGVKFDTRNFRLGWDPETKL
IMPGVYTYEAFHPDIVLLPGCGVDFTESRLSNLLGIRKRHPFQEGFKIMYEDLEGGNIPALLDVTAYEESKKDITTE
TGEKAVVKTTTVAVAEETSEDDNITRGDTYITEKQKREAAAAELLLMSEVKKELKIQPLEKDSKNRSYNVLEDKINT
AYRSWYLSYNYGNPEKGIRSWTLLTTSDVTCGAEQVYWSLPDMMQDPITFRSSRQVNNYPVVGAELMPVFSKSFYNE
QAVYSQQLRQSTSLTHVENRFPENQILIRPPAPTITTISENVPALTDHGTLPLRSSIRGVQRVTVTDARRRTCPYVY
KALGIVAPRVLSSRTF

GenBank Accession No. AAW33444 (SEQ ID NO: 228)

MATPSMMPQWAYMHIAGQDASEYLSPGLVQFARATDTYFSMGNKFRNPTVAPTHDVTTDRSQRLMLRFVPVDREDNT
YSYKVRYTLAVGDNRVLDMASTFFDIRGVLDRGPSFKPYSGTAYNSLAPKGAPNTCQWKDSDSKMHTFGVAAMPGVT
GKKIEADGLPIGIDSTSGTDTVIYADKTFQPEPQVGNASWVDANGTEEKYGGRALKDTTKMKPCYGSFAKPINKEGG
QANLKDSETAATTPNYDIDLAFFDNKNIAANYDPDIVMYTENVDLQTPDTHIVYKPGTEDISSESNLGQQAMPNRPN
YIGFRDNFIGLMYYNSTGNMGVLAGQASQLNAVVDLQDRNTELSYQLLLDSLGDRTRYFSMWNQAVDSYDPDVRIIE
NHGVEDELPNYCFPLNGVGFTDTYQGVKVKTDAVAGTSGTQWDKDDTTVSTANEIHGGNPFAMEINIQANLWRSFLY
SNVALYLPDSYKYTPSNVTLPENKNTYDYMNGRVVPPSLVDTYVNIGARWSLDAMDNVNPFNHHRNAGLRYRSMLLG
NGRYVPFHIQVPQKFFAVKNLLLLPGSYTYEWNFRKDVNMVLQSSLGNDLRVDGASISFTSINLYATFFPMAHNTAS
TLEAMLRNDTNDQSFNDYLSAANMLYPIPANATNIPISIPSRNWAAFRGWSFTRLKTKETPSLGSGFDPYFVYSGSI
PYLDGTFYLNHTFKKVSIMFDSSVSWPGNDRLLSPNEFEIKRTVDGEGYNVAQCNMTKDWFLVQMLANYNIGYQGFY
IPEGYKDRMYSFFRNFQPMSRQVVDEVNYTDYKAVTLPYQHNNSGFVGYLAPTMRQGEPYPANYPYPLIGTTAVKSV
TQKKFLCDRTMWRIPFSSNFMSMGALTDLGQNLLYANSAHALDMTFEVDPMDEPTLLYLLFEVFDVVRVHQPHRGVI
EAVYLRTPFSAGNATT

GenBank Accession No. AY601633 (SEQ ID NO: 204)

CTATCTATATAATATACCTTATAGATGGAATGGTGCCAATATGCAAATGAGGTAATTTAAAAAAGTGCGCGCTGTGT
GGTGATTGGCTGCGGGGTGAACGGCTAAAAGGGGCGGGCAATGCTGGGAGGTGACGTAACTTATGTAGGAGGAGTTA
TGTTGCAAGTTATCGCGGTAAAGGTGACGTAAAACGAGGTGTGGTTTGGACACGGAAGTAGACAGTTTTCCCACGCT
TACTGACAGGATATGAGGTAGTTTTGGGCGGATGCAAGTGAAAATTCTCCATTTTCGCGCGAAAACTGAATGAGGAA
GTGAATTTCTGAGTCATTTCGCGGTTATGACAGGGTGGAGTATTTGCCGAGGGCCGAGTAGACTTTGACCGTTTACG
TGGAGGTTTCGATTACCGTGTTTTTCACCTAAATTTCCGCGTACGGTGTCAAAGTCCTGTGTTTTTACGTAGGTGTC
AGCTGATCACTAGGGTATTTAAACCTGTCGAGTTCCGTCAAGAGGCCACTCTTGAGTGCCAGCGAGAAGAGTTTTCT
CCTCCGCGCTGCGAGTCAGTTTTGCGCTTTGAAAATGAGACACCTGCGATTCCTGCCACAGGAGATTATCTCCAGCG
AGACCGGGATCGAAATACTGGAGTTTGTGGTAAATACCCTGATGGGAGATGACCCGGAACCGCCAGTGCAGCCTTTC
GATCCACCTACGCTGCACGATCTGTATGATTTAGAGGTAGACGGGCCTGATGATCCCAATGAGGAAGCTGTAAATGG
GTTTTTTACTGATTCTATGCTGCTAGCTGCCGATGAAGGATTGGACATAAACCCTCCTCCTGGGACCCTTGATACCC
CAGGGGTGGTTGTGGAAAGCGGCAGAGGTGGGAAAAAATTGCCTGATCTGGGAGCAGCTGAAATGGACTTGCGTTGT
TATGAAGAGGGTTTTCCTCCGAGTGATGATGAAGATGGGGAAACTGAACAGTCCATCCATACCGCAGTGAATGAGGG
AGTAAAAGCTGCCAGCGATGTTTTTAAGTTGGACTGTCCGGAGCTGCCTGGACATGGCTGTAAGTCTTGTGAATTTC
ACAGGAATAACACTGGAATGAAAGAACTATTGTGCTCGCTTTGCTATATGAGAATGCACTGCCACTTTATTTACAGT
AAGTGTATTTAAGTGAAATTTAAAGGAATAGTGTAGCTGTTTAATAACTGTTGAATGGTAGATTTATGTTTTTACTT
GCGATTTTTTGTAGGTCCTGTGTCTGATGATGAGGCGCCTTCTCCTGATTCAACTACCTCACCTCCTGAAATTCAGG
CGCCCGTACCTGCAAACGTATGCAAGCCCATTCCTGTGAAGCCTAAGTGTGGGAAACGCCCTGCTGTGGATAAGCTT
GAGGACTTGTTGGAGGGTGGGGATGGACCTTTGGACCTTAGTACCCGGAAACTGCCAAGACAATGAGTGCCCTGCAG
CTGTGTTTATTTAATGTGACGTCATGTAATAAAATTATGTCAGCTGCTGAGTGTTTTATTGCTTCTTGGGTGGGGAC
TTGGATATATAAGTAGGAGCAGATCTGTGTGGTTAGCTCATAGCAACCTGCTGCCATCCATGGAGGTTTGGGCTATC
TTGGAAGACCTGAGACAGACTAGGCTACTGCTAGAAAACGCCTCGGACGGAGTCTCTGGCTTTTGGAGATTCTGGTT
CGGTGGTGATCTAGCTAGGCTAGTGTTTAGGATAAAACAGGACTACAGGGAAGAATTTGAAAAGTTATTGGACGACA
GTCCAGGACTTTTTGAAGCTCTTAACTTGGGCCACCAGGCTCATTTTAAGGAGAAGGTTTTATCAGTTTTAGATTTT
TCTACTCCTGGTAGAACTGCTGCTGCTGTAGCTTTTCTTACTTTTATATTGGATAAATGGATCCGCCAAACCCACTT
CAGCAAGGGATACGTTTTGGATTTCATAGCAGCAGCTTTGTGGAGAACATGGAAGGCTCGCAGGATGAGGACAATCT
TAGATTACTGGCCAGTGCAGCCTCTGGGAGTAGCAGGGATACTGAGACACCCACCGGCCATGCCAGCGGTTCTGGAG
GAGGAGCAGCAGGAGGACAATCCGAGAGCCGGCCTGGACCCTCCGGTGGAGGAGTAGCTGACCTGTTTCCTGAACTG
CGACGGGTGCTTACTAGGTCTACGTCCAGTGGACAGGACAGGGGCATTAAGAGGGAGAGGAATCCTAGTGGGAATAA
TTCAAGAACCGAGTTGGCTTTAAGTTTAATGAGCCGTAGGCGTCCTGAAACTGTTTGGTGGCATGAGGTTCAGAGCG
AAGGCAGGGATGAAGTTTCAATATTGCAGGAGAAATATTCACTAGAACAACTTAAGACCTGTTGGTTGGAACCTGAG
GATGATTGGGAGGTGGCCATTAGGAATTATGCTAAGATATCTCTGAGGCCTGATAAACAGTATAGAATTACTAAGAA
GATTAATATTAGAAATGCATGCTACATATCAGGGAATGGGGCAGAGGTTATAATAGATACCCAAGATAAAGCAGCTT
TTAGATGTTGTATGATGGGTATGTGGCCAGGGGTTGTCGGCATGGAAGCAGTAACATTTATGAATATTAGGTTTAAA
GGGGATGGGTATAATGGCATTGTATTTATGGCTAACACTAAGCTGATTCTACATGGTTGTAGCTTTTTTGGGTTTAA
TAATACTTGTGTAGAAGCTTGGGGGCAAGTTGGTGTGAGGGGTTGTAGTTTTTATGCATGCTGGATTGCAACATCAG
GTAGGGTCAAGAGTCAGTTGTCTGTGAAGAAATGCATGTTTGAGAGATGTAATCTTGGCATACTGAATGAAGGTGAA
GCAAGGGTCCGCCACTGCGCAGCTACAGAAACTGGCTGCTTCATTCTAATAAAGGGAAATGCCAGTGTGAAGCATAA
TATGATCTGTGGACATTCGAATGAGAGGCCTTATCAGATGCTGACCTGCGCTGGTGGACATTGCAATATTCTTGCTA
CCGTGCATATCGTTTCCCATGCACGCAAGAAATGGCCTGTATTTGAACATAATGTGATTACCAAGTGCACCATGCAC
ATAGGTGGTCGCAGGGGAATGTTTATGCCTTACCAGTGTAACATGAATCATGTGAAGGTGATGTTGGAACCAGATGC
CTTTTCCAGAGTGAGCTTAACAGGAATCTTTGATATGAATATTCAACTATGGAAGATCCTGAGATATGATGACACTA
AACCGAGGGTGCGCGCATGCGAATGCGGAGGCAAGCATGCTAGATTCCAGCCGGTGTGCGTGGATGTGACTGAAGAC
CTGAGACCCGATCATTTGGTGCTTGCCTGCACTGGAGCGGAGTTCGGTTCTAGTGGTGAAGAAACTGACTAAAGTGA
GTAGTGGGGCAATATGTGGATGGGGACTTTCAGGTTGGTAAGGTGGACAGATTGGGTAAATTTTGTTAATTTCTGTC
TTGCAGCTGCCATGAGTGGAAGCGCTTCTTTTGAGGGGGGAGTATTTAGCCCTTATCTGACGGGCAGGCTCCCATCA
TGGGCAGGAGTTCGTCAGAATGTCATGGGATCCACTGTGGATGGGAGACCCGTCCAGCCCGCCAATTCCTCAACGCT
GACCTATGCCACTTTGAGTTCGTCATCATTGGATGCAGCTGCAGCCGCCGCCGCTACTGCTGCCGCCAACACCATCC
TTGGAATGGGCTATTACGGAAGCATCGTTGCCAATTCCAGTTCCTCTAATAACCCTTCAACCCTGGCTGAGGACAAG
CTGCTTGTTCTCTTGGCTCAGCTCGAGGCCTTAACCCAACGCTTAGGCGAACTGTCTAAGCAGGTGGCCCAGTTGCG
TGAGCAAACTGAGTCTGCTGTTGCCACAGCAAAGTCTAAATAAAGATCTCAAATCAATAAATAAAGAAATACTTGTT
ATAAAAACAAATGAATGTTTATTTGATTTTTCGCGCGCGGTATGCCCTGGACCATCGGTCTCGATCATTGAGAACTC
GGTGGATCTTTTCCAGTACCCTGTAAAGGTGGGATTGAATGTTTAGATACATGGGCATTAGTCCGTCTCGGGGGTGG
AGATAGCTCCATTGAAGAGCCTCTTGCTCCGGGGTAGTGTTATAAATCACCCAGTCATAGCAAGGTCGGAGTGCATG
GTGTTGCACAATATCTTTTAGGAGCAGACTAATTGCAACGGGGAGGCCCTTAGTGTAGGTGTTTACAAATCTGTTGA
GCTGGGACGGGTGCATCCTGGGTGAAATTATATGCATTTTGGACTGGATCTTGAGGTTGGCAATGTTGCCGCCTAGA
TCCCGTCTCGGATTCATATTGTGCAGAACCACCAAGACAGTGTATCCGGTGCACTTGGGAAATTTATCATGCAGCTT
AGAGGGAAAAGCATGAAAAAATTTGGAGACGCCTTTGTGACCCCCCAGATTCTCCATGCACTCATCCATAATGATAG
CGATGGGGCCGTGGGCAGCGGCACGGGCGAACACGTTCCGGGGGTCTGAAACATCATAGTTATGCTCCTGAGTCAGG
TCATCATAAGCCATTTTAATAAACTTTGGGCGGAGGGTGCCAGATTGGGGGATGAAAGTTCCCTCTGGCCCGGGAGC
ATAGTTTCCCTCACATATTTGCATTTCCCAGGCTTTCAGTTCCGAGGGGGGGATCATGTCCACCTGCGGGGCTATAA
AAAATACCGTTTCTGGAGCCGGGGTGATTAACTGGGATGAGAGCAAATTCCTAAGCAGCTGAGACTTGCCGCACCCA
GTGGGACCGTAAATGACCCCAATTACGGGTTGCAGATGGTAGTTTAGGGAGCGACAGCTGCCGTCCTCCCGGAGCAG
GGGGGCCACTTCGTTCATCATTTCCCTTACATGGATATTTTCCCGCACCAAGTCCGTTAGGAGGCGCTCTCCCCCAA
GGGATAGAAGCTCCTGGAGCGAGGAGAAGTTTTTCAGCGGCTTCAGCCCGTCAGCCATGGGCATTTTGGAAAGAGTC
TGTTGCAAGAGCTCGAGCCGGTCCCAGAGCTCGGTGATGTGCTCTATGGCATCTCGATCCAACAGACCTCCTCGTTT
CGCGGGTTGGGACGGCTCCTGGAGTAGGGAATCAGACGATGGGCGTCCAGCGCTGCTAGGGTCCGATCCTTCCATGG
TCGCAGCGTCCGAGTCAGGGTTGTTTCCGTCACGGTGAAGGGGTGCGCGCCTGGTTGGGCGCTTGCGAGGGTGCGCT
TCAGACTCATCCTGCTGGTCGAGAACCGCTGCCGATCGGCGCCCTGCAGGTCGGCCAGGTAGCAGTTTACCATGAGT
TCGTAGTTGAGCGCCTCGGCCGCGTGGCCTTTGGCACGGAGCTTACCTTTGGAAGTTTTATGGCAGGCGGGGCAGTA
GATACATTTGAGGGCATACAGCTTGGGCGCGAGGAAAATGGATTCGGGGGAGTATGCATCCGCACCGCAGGAGGCGC
AGACGGTTTCGCACTCCACGAGCCAGGTCAGATCCGGCTCATCGGGGTCAAAAACAAGTTTTCCGCCATGTTTTTTG
ATGCGTTTCTTACCTTTGGTTTCCATGAGTTCGTGTCCCCGCTGGGTGACAAAGAGGCTGTCCGTGTCCCCGTAGAC
CGACTTTATGGGCCTGTCCTCGAGCGGAGTGCCTCGGTCCTCTTCGTAGAGGAACCCAGCCCACTCTGATACAAAAG
CGCGTGTCCAGGCCAGCACAAAGGAGGCCACGTGGGAGGGGTAGCGGTCGTTGTCAACCAGTGGGTCCACCTTCTCT
ACGGTATGTAAACACATGTCCCCCTCCTCCACATCCAAGAATGTGATTGGCTTGTAAGTGTAGGCCACGTGACCAGG
GGTCCCCGCCGGGGGGGTATAAAAGGGGGCGGGCCTCTGTTCGTCCTCACTGTCTTCAGGATCGCTGTCCAGGAGCG
CCAGCTGTTGGGGTAGGTATTCCCTCTCGAAGGCGGGCATGACCTCTGCACTCAGGTTGTCAGTTTCTAGGAACGAG
GAGGATTTGATATTGACAGTACCAGCAGAGACGCCTTTCATAAGACTCTCGTCCATCTGGTCAGAAAACACAATCTT
CTTGTTGTCCAGCTTGGTGGCAAATGATCCATAAAGGGCATTGGACAGAAGCTTGGCGATGGAGCGCATGGTTTGGT
TCTTTTCCTTGTCCGCGCGCTCCTTGGCGGCGATGTTAAGCTGGACGTACTCGCGCGCCACACATTTCCATTCAGGG
AAGATGGTTGTCAGTTCATCCGGAACTATTCTGACTCGCCATCCCCTATTGTGCAGGGTTATCAGATCCACACTGGT
GGCCACCTCGCCTCGGAGGGGCTCATTGGTCCAGCAGAGTCGACCTCCTTTTCTTGAACAGAAAGGTGGGAGGGGGT
CTAGCATGAACTCATCAGGGGGGTCCGCATCTATGGTAAATATTCCCGGTAGCAAATCTTTGTCAAAATAGCTGATG
GTGGCGGGATCATCCAAGGTCATCTGCCATTCTCGAACTGCCAGCGCGCGCTCATAGGGGTTAAGAGGGGTGCCCCA
GGGCATGGGGTGGGTGAGCGCGGAGGCATACATGCCACAGATATCGTAGACATAGAGGGGCTCTTCGAGGATGCCGA
TGTAAGTGGGATAACAGCGCCCCCCTCTGATGCTTGCTCGCACATAGTCATAGAGTTCATGTGAGGGGGCGAGAAGA
CCCGGGCCCAGATTGGTGCGGTTGGGTTTTTCCGCCCTGTAAACGATCTGGCGAAAGATGGCATGGGAATTGGAAGA
GATAGTAGGTCTCTGGAATATGTTAAAATGGGCATGAGGTAGGCCTACAGAGTCCCTTATGAAGTGGGCATATGACT
CTTGCAGCTTGGCTACCAGCTCGGCGGTGACGAGTACATCCAGGGCACAGTAGTCGAGAGTTTCCTGGATGATGTCA
TAACGCGGTTGGCTTTTCTTTTCCCACAGCTCGCGGTTGAGAAGGTATTCTTCGCGATCCTTCCAGTACTCTTCGAG
GGGAAACCCGTCTTTTTCTGCACGGTAAGAGCCCAACATGTAGAACTGATTGACTGCCTTGTAGGGACAGCATCCCT
TCTCCACTGGAAGAGAGTATGCTTGGGCTGCATTGCGCAGCGAGGTATGAGTGAGGGCAAAAGTGTCCCTGACCATG
ACTTTGAGGAATTGATACTTGAAGTCAATGTCATCACAGGCCCCCTGTTCCCAGAGTTGGAAGTCCACCCGCTTCTT
GTAGGCGGGGTTGGGCAAAGCGAAAGTAACATCATTGAAGAGGATCTTGCCGGCCCTGGGCATGAAATTTCGGGTGA
TTTTGAAAGGCTGAGGGACCTCTGCTCGGTTATTGATAACCTGAGCGGCCAAGACGATCTCATCAAAGCCATTGATG
TTGTGCCCCACTATGTACAGTTCTAAGAATCGAGGGGTGCCCCTGACATGAGGCAGCTTCTTGAGTTCTTCAAAAGT
GAGATCTGTAGGGTCAGTGAGAGCATAGTGTTCGAGGGCCCATTCGTGCACGTGAGGGTTCGCTTTGAGGAAGGAGG
ACCAGAGGTCCACTGCCAGTGCTGTTTGTAACTGGTCCCGGTACTGACGAAAATGCTGCCCGACTGCCATCTTTTCT
GGGGTGACGCAATAGAAGGTTTGGGGGTCCTGCCGCCAGCGATCCCACTTGAGTTTCATGGCGAGGTCATAGGCGAT
GTTGACGAGCCGCTGGTCTCCAGAGAGTTTCATGACCAGCATGAAGGGGATTAGCTGCTTGCCAAAGGACCCCATCC
AGGTGTAGGTTTCCACATCGTAGGTGAGGAAGAGCCTTTCTGTGCGAGGATGAGAGCCAATCGGGAAGAACTGGATC
TCCTGCCACCAGTTGGAGGAATGGCTGTTGATGTGATGGAAGTAGAACTCCCTGCGACGCGCCGAGCATTCATGCTT
GTGCTTGTACAGACGGCCGCAGTACTCGCAGCGATTCACGGGATGCACCTCATGAATGAGTTGTACCTGACTTCCTT
TGACGAGAAATTTCAGTGGAAAATTGAGGCCTGGCGTTTGTACCTCGCGCTCTACTATGTTGTCTGCATCGGCATGA
CCATCTTCTGTCTCGATGGTGGTCATGCTGACGAGCCCTCGCGGGAGGCAAGTCCAGACCTCGGCGCGGCAGGGGCG
GAGCTCGAGGACGAGAGCGCGCAGGCCGGAGCTGTCCAGGGTCCTGAGACGCTGCGGAGTCAGGTTAGTAGGCAGTG
TCAGGAGATTGACTTGCATGATCTTTTCGAGGGCGTGAGGGAGGTTCAGATGGTACTTGATCTCCACGGGTCCGTTG
GTGGAGATGTCGATGGCTTGCAGGGTTCCGTGCCCCTTGGGTGCTACCACCGTGCCCTTGTTTTTCCTTTTGGGCGG
CGGTGGCTCTGTTGCTTCTTGCATGTTTAGAAGCGGTGTCGAGGGCGCGCACCGGGCGGCAGGGGCGGTTCGGGACC
CGGCGGCATGGCTGGCAGTGGTACGTCGGCGCCGCGCGCGGGTAGGTTCTGGTACTGTGCTCTGAGAAGACTCGCAT
GCGCGACGACGCGGCGGTTGACATCCTGGATCTGACGCCTCTGGGTGAAAGCTACCGGCCCCGTGAGCTTGAACCTG
AAAGAGAGTTCAACAGAATCAATCTCGGTATCGTTGACGGCGGCTTGCCTAAGGATTTCTTGCACGTCGCCAGAGTT
GTCCTGGTAGGCGATCTCGGCCATGAACTGCTCGATCTCTTCCTCTTGAAGATCTCCGCGGCCCGCTCTCTCGACGG
TGGCCGCCAGGTCGTTGGAGATGCGCCCAATGAGTTGAGAGAATGCATTCATGCCCGCCTCGTTCCAGACGCGGCTG
TAGACCACAGCCCCCACGGGATCTCTCGCGCGCATGACCACCTGGGCGAGGTTGAGCTCCACGTGGCGGGTGAAGAC
CGCATAGTTGCATAGACGCTGGAAAAGGTAGTTGAGTGTGGTGGCGATGTGCTCGGTGACGAAGAAATACATGATCC
ATCGTCTCAGCGGCATCTCGCTGACATCGCCCAGCGCTTCCAAGCGCTCCATGGCCTCGTAGAAGTCCACGGCAAAG
TTGAAAAACTGGGAGTTACGCGCGGACACGGTCAACTCCTCTTCCAGAAGACGGATGAGTTCGGCGACGGTGGTGCG
CACCTCGCGCTCGAAAGCCCCTGGGATTTCTTCCTCAATCTCTTCTTCTTCCACTAACATCTCTTCCTCTTCAGGTG
GGGCTGCAGGAGGAGGGGGAACGCGGCGACGCCGGCGGCGCACGGGCAGACGGTCGATGAATCTTTCAATGACCTCT
CCGCGGCGGCGGCGCATGGTCTCGGTGACGGCACGACCGTTCTCCCTGGGTCTCAGAGTGAAGACGCCTCCGCGCAT
CTCCCTGAAGTGGTGACTGGGAGGCTCTCCGTTGGGCAGGGACACCGCGCTGATTATGCATTTTATCAATTGCCCCG
TAGGTACTCCGCGCAAGGACCTGATCGTCTCAAGATCCACGGGATCTGAAAACCTTTCGACGAAAGCGTCTAACCAG
TCGCAATCGCAAGGTAGGCTGAGCACTGTTTCTTGCGGGGGGGGGGGGCTAGACGCTCGGTCGGGGTTCTCTCTTTC
TTCTCCTTCCTCCTCTTTGGAGGGTGAGACGATGCTGCTGGTGATGAAATTAAAATAGGCAGTTTTGAGACGGCGGA
TGGTGGCGAGGAGCACCAGGTCTTTGGGTCCGGCTTGTTGGATGCGCAGGCGATGGGCCATTCCCCAAGCATTATCC
TGACATCTGGCCAGATCTTTATAGTAGTCTTGCATGAGTCGTTCCACGGGCACTTCTTCTTCGCCCGCTCTGCCATG
CATGCGAGTGATCCCGAACCCGCGCATGGGCTGGACAAGTGCCAGGTCCGCTACAACCCTTTCTGCGAGGATGGCTT
GCTGCACCTGGGTGAGGGTGGCTTGGAAGTCGTCAAAGTCCACGAAGCGGTGGTAAGCCCCGGTGTTGATTGTGTAG
GAGCAGTTGGCCATGACTGACCAGTTGACTGTCTGGTGCCCCGGACGCACAATCTCGGTGTACTTGAGGCGCGAGTA
GGCGCGGGTGTCAAAGATGTAATCGTTACAGGTGCGCACCAGGTACTGGTAGCCGATAAGAAAGTGCGGCGGCGGCT
GGCGGTATAGGGGCCATCGCTCTGTAGCCGGGGCGCCAGGGGCGAGGTCTTCCAGCATGAGGCGGTGATAACCGTAG
ATGTACCTGGACATCCAGGTGATACCGGAGGCGGTGGTGGATGCCCGCGGGAACTCGCGTACGCGGTTCCAGATGTT
GCGCAGCGGCATGAAGTAGTTCATGGTAGGCACGGTTTGGCCCGTGAGACGTGCACAGTCGTTGATGCTCTAGACAT
ACGGGCAAAAACGAAAGCGGTCAGCGGCTCGTCTCCGTGGCCTGGAGGCTAAGCGAACGGGTTGGGCTGCGCGTGTA
CCCCGGTTCGAATCTCGGATCAGGCTGGAGCCGCAGCTAACGTGGTACTGGCACTCCCGTCTCGACCCAGGCCTGCA
CAAAACCTCCAGGATACGGAGGCGGGTCGTTTTTTTTTTTTTTTGCTTTTTCCTGGATGGGAGCCAGTGCTGCGTCA
AGCTTTAGAACACTCAGTTCTCGGGGCTGGGAGTGGCTCGCGCCCGTAGTCTGGAGAATCAATCGCCAGGGTTGCGT
TGCGGTGTGCCCCGGTTCGAGTCTTAGCGCGCCGGATCGGCCGGTTTCCGCGACAAGCGAGGGTTTGGCAGCCCCGT
CATTTCTAAGACCCCGCCAGCCGACTTCTCCAGTTTACGGGAGCGAGCCCTCTTTTTTTGTTTTTTTGTTGCCCAGA
TGCATCCCGTGCTGCGACAGATGCGCCCCCAGCAACAGCCCCCTTCTCAGCAGCAGCTACAACAACAGCCACAAAAG
GCTCTTCCTGCTCCTGTAACTACTGCGGCTGCAGCCGTCAGCGGCGCGGGGCAGCCCGCCTATGATCTGGACTTGGA
AGAGGGCGAGGGACTGGCGCGCCTGGGCGCACCATCGCCCGAGCGGCACCCGCGGGTGCAACTGAAAAAGGACTCTC
GCGAGGCGTACGTGCCCCAGCAGAACCTGTTCAGGGACAGGAGCGGCGAGGAGCCTGAGGAAATGCGAGCTTCCCGC
TTTAACGCGGGTCGCGAACTGCGTCACGGTCTGGACCGAAGACGGGTGCTGCGTGATGATGATTTTGAAGTCGATGA
AGTGACAGGAATAAGTCCTGCTAGGGCACATGTGGCCGCGGCCAACCTAGTATCAGCTTACGAGCAGACCGTGAAGG
AGGAGCGCAACTTTCAAAAATCTTTCAACAACCATGTGCGCACCCTGATTGCCCGCGAGGAAGTGACACTGGGACTG
ATGCACCTGTGGGACCTGATGGAAGCCATTACCCAGAACCCCACCAGCAAACCTCTAACCGCTCAGCTGTTTCTGGT
GGTGCAACATAGTAGAGACAATGAGGCATTTAGGGAGGCGCTGTTGAACATTACTGAGCCCGAGGGGAGATGGTTGT
ATGATCTTATCAATATTCTGCAAAGTATAATAGTGCAAGAACGTAGCCTGGGTCTAGCTGAGAAGGTGGCTGCTATT
AACTACTCGGTCTTGAGCCTGGGCAAGCACTACGCTCGCAAGATCTACAAAACCCCATACGTACCTATAGACAAGGA
GGTGAAGATAGATGGGTTTTATATGCGCATGACTCTCAAGGTGCTGACCTTGAGTGACGATCTGGGAGTGTACCGCA
ACGACAGGATGCACCGCGCAGTGAGCGCCAGCAGAAGGCGTGAGCTGAGCGACAGAGAACTTATGCACAGCTTGCAA
AGAGCTCTGACTGGGGCTGGAACCGAGGGGGAGAACTACTTTGACATGGGAGCGGACTTGCAATGGCAGCCCAGTCG
CAGGGCCCTGGACGCAGCAGGGTATGAGCTTCCTTACATAGAAGAGGTGGATGAAGGCCAGGATGAGGAGGGCGAGT
ACCTGGAAGACTGATGGCGCGACCATCCATATTTTTGCTAGATGGAACAGCAGGCACCGGACCCCGCAAAACGGGCG
GCGCTACAGAGCCAGCCGTCCGGCATTAACTCCTCGGACGATTGGAGCCAGGCCATGCAACGCATCATGGCGCTGAC
GACCCGCAACCCCGAAGCCTTTAGGCAGCAACCCCAGGCCAACCGCCTTTCTGCTATCCTGGAGGCCGTAGTGCCCT
CCCGCTCCAACCCCACACACGAGAAGGTCCTGGCCATCGTGAACGCGCTGGTGGAGAACAAAGCCATACGTCCCGAT
GAGGCTGGGCTGGTATACAATGCCCTATTGGAGCGCGTAGCCCGCTACAACAGCAGCAACGTGCAGACCAACCTGGA
CCGGATGGTGACCGATGTGCGCGAGGCCGTGTCTCAGCGCGAGCGGTTCCAGCGAGACGCCAATTTAGGGTCGCTGG
TGGCTTTGAACGCCTTCCTCAGCACTCAGCCTGCCAACGTGCCTCGCGGTCAGCAAGACTACACAAACTTTCTAAGT
GCATTGAGACTCATGGTGGCCGAAGTCCCTCAAAGCGAAGTGTACCAGTCCGGGCCAGACTACTTTTTCCAGACCAG
CAGACAGGGCTTGCAGACAGTGAACCTGAGCCAGGCTTTTAAGAACCTGAATGGTCTGTGGGGAGTGCGCGCCCCAG
TGGGAGATCGGGCGACCGTGTCTAGCTTGCTGACCCCCAACTCCCGCCTACTACTGCTCTTGGTAGCCCCATTCACT
GACAGCGGTAGCATCGACCGTAATTCGTACTTGGGCTATCTGTTGAACCTGTATCGCGAGGCCATAGGGCAAACTCA
GGTAGATGAGCAAACCTATCAAGAAATTACCCAAGTGAGCCGCGCTCTGGGTCGGGAGGACACTGGCAGCTTGGAAG
CCACCTTAAACTTCTTGCTGACCAACCGGTCGCAGAAGATCCCTCCTCAGTATGCGCTTACCGCGGAGGAGGAACGG
ATCCTGAGATACGTGCAGCAGAGCGTGGGACTGTTCCTAATGCAGGAGGGGGCGACTCCTACTGCTGCGCTCGATAT
GACAGCCCGAAACATGGAGCCCAGCATGTATGCCAGTAACCGGCCTTTTATCAATAAACTGCTAGACTACTTACACA
GGGCGGCTGCTATGAACTCTGATTATTTCACCAATGCTATCCTGAACCCCCATTGGCTGCCCCCACCTGGGTTCTAT
ACGGGCGAGTATGACATTGCCCGACCCAATGACGGGTTTTTATGGGACGATGTGGACAGTAGTGTTTTCTCCCCGCC
TCCTGGTTATAACACTTGGAAGAAGGAAGGTGGCGATAGAAGGCACTCTTCCGTGTCACTGTCCGGGGCAACGGGTG
CTGCCGCAGCGGCTCCCGAGGCCGCAAGTCCTTTCCCTAGTTTGCCATTTTCGCTAAACAGTGTACGCAGCAGTGAG
CTGGGAAGAATAACCCGTCCTCGCTTGATCGGCGAGGAGGAGTATTTGAACGACTCCCTGTTGAGACCCGAGAGGGA
GAAGAATTTCCCCAACAACGGGATAGAAAGCTTGGTTGACAAAATGAACCGCTGGAAGACGTACGCGCACGATCCCC
GGGCGCTGGGGGATAGCCGGGGCAGCGCTACCCGTAAACGCCAGTGGCACGACAGGCAGCGGGGCCTGGTGTGGGCC
GATGATGATTCCGCCGACGACAGCAGCGTGTTGGACTTGGGTGGGAGTGGTGGTAACCCGTTCGCTCACCTGCGCCC
CCGCGTCGGGCGCCTGATGTAAGAAACCGAAAATAAATACTCACCAAGGCCATGGCGACCAGCGTGCGTTCGTTTCT
TCTCTGTTATATCTAGTATGATGAGGCGAACCGTGCTAGGCGGAGCGGTGGTGTATCCGGAGGGTCCTCCTCCTTCG
TACGAGAGCGTGATGCAGCAGGCGGCGGCGGCGACGATGCAGCCACCACTGGAGGCTCCCTTTGTACCCCCTCGGTA
CCTGGCACCTACGGAGGGGAGAAACAGCATTCGTTACTCGGAGCTGGCACCATTGTATGATACCACCCGGTTGTATT
TGGTGGACAACAAGTCCGCGGACATCGCCTCACTGAACTATCAGAACGACCACAGCAACTTCCTCACCACGGTGGTG
CAAAACAATGACTTTACCCCCACGGAGGCCAGCACCCAGACCATCAACTTTGACGAGCGGTCGCGATGGGGCGGTCA
GCTGAAGACTATCATGCACACCAACATGCCCAACGTGAACGAGTACATGTTTAGCAACAAGTTCAAAGCTCGGGTGA
TGGTGTCTAGAAAGGCTCCTGAAGGTGTCACAGTAGATGACAATTATGATCACAAGCAGGATATTTTGGAATATGAG
TGGTTTGAGTTTACTCTACCGGAAGGGAATTTCTCAGCCACAATGACCATTGACCTAATGAACAATGCCATCATTGA
TAATTACCTTGAAGTGGGCAGACAGAATGGAGTGTTAGAGAGTGACATTGGTGTTAAATTTGACACCAGGAACTTTA
GACTGGGTTGGGATCCGGAAACTAAGTTGATTATGCCTGGGGTTTACACCTATGAGGCATTCCATCCTGACATTGTA
TTGTTGCCTGGTTGCGGAGTTGACTTTACTGAAAGTCGCCTTAGTAACTTGCTTGGTATCAGGAAAAGACACCCATT
CCAGGAGGGTTTTAAGATCTTGTATGAGGATCTTGAAGGGGGTAATATCCCGGCCCTGTTGGATGTAGAAGCCTATG
AGAACAGTAAGAAAGAACAAGAAGCCAAAACAGAAGCCGCTAAAGCTGCTGCTATTGCTAAAGCCAACATAGTTGTC
AGCGACCCTGTAAGGGTGGCTAATGCAGAAGAAGTCAGAGGAGACAACTATACAGCTTCATCTGTTGCAACTGACGA
ATCGCTATTGGCTGCTGTGGCCGAAACTACAGAGACAAAACTCACTATTAAACCTGTAGAAAAAGACAGCAAGAGTA
GAAGTTACAATGTCTTGGAAGATAAAGTGAATACAGCCTACCGCAGCTGGTACCTGTCCTACAACTATGGTGACCCT
GAAAAAGGAGTCCGTTCCTGGACACTGCTCACCACCTCGGATGTCACCTGTGGAGCAGAGCAGGTGTACTGGTCGCT
CCCAGACATGATGCAGGACCCTGTCACATTCCGTTCCACGAGACAAGTCAGCAACTATCCAGTGGTAGGTGCAGAGC
TCATGCCGGTCTTCTCAAAGAGTTTCTACAACGAGCAAGCCGTGTACTCCCAGCAGCTTCGCCAGTCCACCTCGCTC
ACGCACGTCTTCAACCGCTTCCCTGAGAACCAGATCCTCATCCGCCCGCCAGCGCCCACCATTACCACCGTCAGTGA
AAACGTTCCTGCTCTCACAGATCACGGGACCCTGCCGTTGCGCAGCAGTATCCGGGGAGTCCAGCGCGTGACCGTTA
CTGACGCCAGACGCCGCACCTGCCCCTACGTCTACAAGGCCCTGGGCATAGTCGCGCCGCGCGTCCTTTCAAGCCGC
ACTTTCTAAAAAAAAAAATGTCCATTCTTATCTCACCTAGTAATAACACCGGTTGGGGCCTGCGCGCGCCAAGCAAG
ATGTACGGAGGTGCTCGCAAACGCTCTACACAGCACCCTGTGCGCGTGCGCGGGCACTTCCGCGCTCCATGGGGCGC
CCTCAAGGGTCGTGCCCGCACTAGAACCACCGTCGATGATGTGATCGACCAGGTGGTGGCCGATGCTCGTAATTATA
CTCCTACTGCACCTACATCTACTGTGGATGCAGTTATTGACAGCGTAGTGGCTGACGCCCGCGCCTATGCTCGCCGG
AAGAGCAGGCGGAGACGCATCGCCAGGCGCCACCGGGCTACTCCCGCTATGCGAGCGGCAAGAGCTCTGCTACGGAG
GGCCAAACGCGTGGGGCGAAGAGCTATGCTTAGAGCGGCCAGACGCGCGGCTTCAGGTGCCAGTGCCGGCAGGTCCC
GCAGGCGCGCAGCCACGGCGGCAGCAGCGGCCATTGCCAACATGGCCCAACCGCGAAGAGGCAATGTGTACTGGGTG
CGCGACGCCACCACCGGCCAGCGCGTGCCCGTGCGCACCCGCCCCCCTCGCTCTTAGAAGATACTGAGCAGTCTCCG
ATGTTGTGTCCCAGCGAGGATGTCCAAGCGCAAATACAAGGAAGAGATGCTCCAGGTCATCGCGCCTGAAATCTACG
GTCCGCCGGTGAAGGATGAAAAAAAGCCCCGCAAAATCAAGCGGGTCAAAAAGGACAAAAAGGAAGAAGATGGCAAT
GATGGTCTGGCGGAGTTTGTACGCGAGTTCGCCCCAAGGCGGCGTGTGCAGTGGCGTGGACGCAAAGTGCGGCCTGT
GCTGAGACCTGGAACCACGGTGGTCTTTACGCCCGGCGAGCGCTCCAGCACTGCTTTTAAGCGGTCCTATGATGAGG
TGTATGGGGATGATGATATTCTGGAGCAGGCGGCCGACCGCCTGGGCGAGTTTGCTTATGGCAAGCGCTCCCGCTCG
AGCCCCAAGGAGGAGGCGGTGTCCATTCCCTTGGACAATGGGAATCCCACCCCTAGTCTCAAGCCAGTCACCCTGCA
GCAAGTGCTGCCCGTGCCTCCACGCAGAGGCAACAAGCGAGAGGGTGAGGATCTGTATCCCACTATGCAATTGATGG
TGCCCAAGCGCCAGCGGCTGGAGGACGTGCTGGAGAAAATGAAAGTGGATCCCGATATACAACCTGAGGTCAAAGTG
AGACCCATCAAGCAGGTGGCGCCAGGTTTGGGAGTACAAACCGTAGACATCAAGATTCCCACCGAGTCCATGGAAGT
CCAAACCGAACCTGCAAAGCCCACAACCACCTCCATTGAGGTGCAAACGGATCCCTGGATGACCGCACCCGTTACAA
CTCCAGCTGCTGTCAACACCACTCGAAGATCCCGGCGAAAGTACGGTCCAGCAAGTTTGCTGATGCCAAATTATGCT
CTGCACCCATCCATTATTCCAACTCCGGGTTACCGAGGCACTCGCTACTACCGCAGCAGGAGCAGCACTTCCCGCCG
TCGCCGCAAAACACCTGCAAGTCGTAGTCACCGTCGTCGCCGCCGCCCCACCAGCAATCTGACTCCCGCTGCTCTGG
TGCGGAGAGTGTATCGCGATGGCCGCGCGGATCCCCTGACGTTGCCGCGCGTACGCTACCATCCAAGCATCACAACT
TAACAACTGTTGCCGCTGCCTCCTTGCAGATATGGCCCTCACTTGCCGCCTTCGTGTCCCCATTACTGGCTACCGAG
GAAGAAACTCGCGCCGTAGAAGAGGGATGTTGGGGCGCGGAATGCGACGCCACAGGCGGCGGCGCGCTATCAGCAAG
AGGCTGGGGGGTGGCTTTCTGCCTGCTCTGATCCCCATCATAGCCGCGGCGATCGGGGCGATACCAGGCATAGCTTC
CGTGGCGGTTCAGGCCTCGCAGCGCCACTGACATTGGAAAAACTTATAAATAAAACAGAATGGACTCTGATGCTCCT
GGTCCTGTGACTATGTTTTTGTAGAGATGGAAGACATCAATTTTTCATCCCTGGCTCCGCGACACGGCACGAGGCCG
TACATGGGCACCTGGAGCGACATCGGCACCAGCCAACTGAACGGGGGCGCCTTCAATTGGAGCAGTATCTGGAGCGG
GCTTAAAAATTTTGGCTCTACCATAAAAACCTATGGGAACAAAGCTTGGAACAGCAGCACAGGGCAGGCATTGAGAA
ATAAGCTTAAAGAGCAAAACTTCCAACAGAAGGTGGTTGATGGAATCGCCTCTGGTATCAATGGGGTGGTGGATCTG
GCCAACCAGGCCGTGCAGAAACAGATAAACAGCCGCATTGACCCGCCGCCGTCAGCCCCGGGTGAAATGGAAGTGGA
GGAAGATCTCCCTCCCCTTGAAAAGCGGGGCGACAAGCGTCCGCGCCCCGATCTGGAGGAGACACTAGTCACACGCT
CAGACGACCCGCCCTCCTACGAGGAGGCAGTGAAGCTTGGAATGCCCACCACCAGACCTGTAGCCCCCATGGCTACC
GGGGTAATGAAACCTTCTCAGTCACACCGACCCGCTACCTTGGACTTGCCTCCCCCTGCTGTTGCAGCGCCTGCTCG
CAAGCCTGTCGCTACCCCGAAGCCCACCACCGTACAGCCCGTCGCCGTAGCCAGACCGCGTCCTGGGGGCACTCCAC
GTCCGAATGCAAACTGGCAGAGTACTCTGAACAGCATCGTGGGTCTGGGCGTGCAAAGTGTAAAGCGCCGTCGCTGC
TTTTAAATTAATATGGAGTAGCGCTTAACTTGCCTGTCTGTGTGTATGTGTCATCATCACGCCGCCGCCGCAGCAAC
AGCAGAGGAGCAAGGAAGAGGTCGCGCGCCGAGGCTGAGTTGATTTCAAGATGGCCACCCCATCGATGCTGCCCCAG
TGGGCATACATGCACATCGCCGGACAGGATGCTTCGGAGTACCTGAGTCCGGGTCTGGTGCAGTTCGCCCGCGCCAC
AGACACCTACTTCAATCTGGGGAACAAGTTTAGGAACCCCACCGTGGCGCCCACCCATGATGTGACCACCGACCGCA
GTCAGCGGCTGATGCTGCGCTTTGTGCCCGTTGACCGGGAAGACAATACCTACGCATACAAAGTTCGATACACCTTG
GCTGTGGGCGACAACAGAGTGCTGGATATGGCCAGCACTTTCTTTGACATTCGGGGTGTGTTGGATAGAGGCCCTAG
CTTCAAGCCATACTCTGGCACTGCTTACAACTCGTTGGCCCCTAAGGGCGCTCCCAATACATCTCAGTGGATTGCTG
AAGGCGTAAAAAAAGAAGATGGGGGATCTGACGAAGAGGAAGAGAAAAATCTCACCACTTACACTTTTGGAAATGCC
CCAGTGAAAGCAGAAGGTGGTGATATCACTAAAGACAAAGGTCTTCCAATTGGTTCAGAAATTACAGACGGCGAAGC
CAAACCAATTTATGCAGATAAACTATACCAACCAGAACCTCAGGTGGGAGATGAAACTTGGACTGACACAGATGGAA
CAACTGAGAAGTATGGTGGTAGAGCTCTAAAGCCAGAAACTAAAATGAAACCCTGCTATGGGTCTTTTGCTAAACCC
ACTAACGTCAAAGGCGGACAGGCAAAACAAAAAACTACTGAACAACCGCAAAACCAGCAGGTTGAATATGATATTGA
CATGAACTTTTTTGATGAAGCGTCACAGAAAGCAAACTTCAGTCCAAAAATTGTGATGTATGCAGAAAATGTAGACT
TGGAAACCCCAGACACTCATGTGGTGTACAAACCTGGTACTTCAGAAGAAAGTTCTCATGCTAATCTGGGTCAACAA
TCTATGCCCAACAGACCCAACTACATTGGCTTTAGAGATAACTTTATTGGACTTATGTACTACAACAGTACTGGCAA
CATGGGAGTGCTGGCAGGTCAAGCATCCCAATTGAATGCGGTGGTTGACTTGCAGGACAGAAACACAGAACTATCAT
ATCAACTACTGCTTGACTCTCTGGGTGACAGAACCAGATACTTCAGCATGTGGAATCAAGCAGTCGATAGCTATGAT
CCTGATGTGCGCATTATTGAAAATCATGGGGTGGAAGATGAGCTTCCCAACTACTGCTTTCCATTGGATGGAGTAGG
GGTACCAATAAGTAGTTACAAAATAATTGAACCAAACGGACAGGGTGCAGATTGGAAAGAGCCTGACATAAATGGAA
CAAGTGAAATTGGACAAGGAAATCTCTTTGCCATGGAAATTAACCTCCAAGCTAATCTCTGGAGAAGTTTTCTTTAT
TCCAATGTGGCTCTGTATCTCCCAGACTCCTACAAATACACCCCAGCCAATGTCACTCTTCCAACTAACACCAACAC
TTATGACTACATGAATGGGCGGGTGGTTCCCCCATCCCTGGTGGATACCTACGTAAACATTGGCGCCAGATGGTCTT
TGGATGCCATGGACAATGTCAACCCCTTTAACCATCACCGCAACGCTGGCCTGCGATACCGGTCCATGCTTTTGGGC
AATGGTCGTTACGTGCCTTTCCACATTCAAGTGCCTCAGAAATTCTTTGCTGTGAAGAACCTGCTGCTTCTACCCGG
TTCTTACACCTACGAGTGGAACTTCAGAAAGGATGTGAACATGGTCCTGCAGAGTTCCCTTGGTAATGATCTCCGGG
TCGATGGTGCCAGCATAAGTTTTACCAGCATCAATCTCTATGCCACCTTCTTCCCCATGGCCCACAACACTGCCTCC
ACCCTTGAAGCCATGCTGCGCAATGACACCAATGATCAATCATTCAATGACTACCTTTCTGCTGCCAACATGCTCTA
CCCCATCCCGGCCAACGCTACCAACGTTCCCATCTCCATTCCCTCTCGCAACTGGGCCGCCTTCAGAGGCTGGTCCT
TCACCAGACTCAAAACCAAGGAGACTCCCTCTTTGGGATCAGGGTTCGATCCCTACTTTGTTTACTCTGGTTCTATA
CCCTACCTGGATGGTACCTTCTACCTTAACCACACTTTCAAGAAAGTCTCCATCATGTTTGACTCTTCAGTGAGCTG
GCCTGGTAATGACAGATTGCTAAGTCCAAATGAGTTCGAAATCAAGCGCACAGTTGATGGGGAAGGCTACAATGTGG
CCCAATGTAACATGACCAAAGACTGGTTCCTGGTCCAGATGCTTGCCAACTACAACATTGGATACCAGGGCTTCTAC
GTTCCTGAGGGTTACAAGGATCGCATGTACTCCTTCTTCAGAAACTTCCAGCCCATGAGTAGACAGGTGGTTGATGA
GATTAACTACAAAGACTATAAAGCTGTCGCCGTACCCTACCAGCATAATAACTCTGGCTTTGTGGGTTACATGGCTC
CTACCATGCGTCAGGGTCAAGCGTACCCTGCTAACTACCCATACCCCCTAATTGGAACCACTGCAGTAACCAGTGTC
ACCCAGAAAAAATTCCTGTGCGACAGGACCATGTGGCGCATCCCATTCTCTAGCAACTTCATGTCCATGGGTGCCCT
TACAGACCTGGGACAGAACTTGCTGTATGCCAACTCGGCCCATGCGCTGGACATGACTTTTGAGGTGGATCCCATGG
ATGAGCCCACCCTGCTTTATCTTCTTTTCGAAGTCTTCGACGTGGTCAGAGTGCACCAGCCACACCGCGGCGTCATC
GAGGCCGTCTACCTGCGCACACCGTTCTCGGCCGGCAACGCCACCACATAAGAAGCCTCTTGCTTCTTGCAAGCAGC
AGCTGCAGCCATGTCATGCGGGTCCGGAAACGGCTCCAGCGAGCAAGAGCTCAAAGCCATCGTCCGAGACCTGGGCT
GCGGACCCTATTTCCTGGGAACCTTTGACAAGCGTTTCCCGGGGTTCATGGCCCCCGACAAGCTCGCCTGCGCCATA
GTCAACACTGCCGGACGCGAGACGGGGGGAGAGCACTGGCTGGCTTTTGGTTGGAACCCGCGCTCCAACACCTGCTA
CCTTTTTGATCCTTTTGGGTTCTCGGATGAGCGACTCAAACAGATTTACCAGTTTGAGTACGAGGGGCTCCTGCGCC
GCAGTGCCCTTGCTACCAAAGACCGCTGCATCACCCTGGAAAAGTCCACCCAGAGCGTGCAGGGCCCGCGCTCAGCC
GCCTGTGGACTTTTTTGCTGTATGTTCCTTCATGCCTTTGTGCACTGGCCCGACCGCCCCATGAACGGAAACCCCAC
CATGAAGTTGCTGACTGGGGTGTCAAACAGCATGCTCCAATCACCCCAAGTCCAGCCCACCCTGCGTCGCAACCAGG
AGGCGCTATATCGCTTCCTAAACACCCACTCATCTTACTTTCGTTCTCACCGCGCACGCATCGAAAGGGCCACCGCG
TTTGACCGTATGGATATGCAATAAGTCATGTAAAACCGTGTTCAATAAAAAGCACTTTATTTTTACATGCACTAAGG
CTCTGGTTTTTTGCTCATTCGTTTTCATCATTCACTCAGAAATCAAATGGGTTCTGGCGTGAGTCAGAGTGACCCGT
GGGCAGGGAGACGTTGCGGAACTGTAACCTGTTCTGCCACTTGAACTCGGGGATCACCAGCTTGGGAACTGGAATTT
CGGGAAAGGTGTCTTGCCACAACTTTCTGGTCAGTTGCAGGGCGCCAAGCAGGTCAGGAGCAGAGATCTTGAAATCA
CAGTTGGGGCCGGCATTCTGGACACGGGAGTTGCGGTACACTGGGTTGCAACACTGGAACACCATCAAGGCTGGGTG
TCTCACGCTTGCCAGCACGGTCGGGTCACTGATGGTAGTCACATCCAAGTCTTCAGCATTGGCCATTCCAAAGGGGG
TCATCTTACAGGTCTGCCTGCCCATCACGGGAGCGCAGCCTGGCTTGTGGTTGCAATCGCAATGAATGGGGATCAGC
ATCATCCTGGCTTGGTCGGGGGTTATCCCTGGGTACACGGCCTTCATGAAGGCTTCGTACTGCTTGAAAGCTTCCTG
AGCCTTACTTCCCTCGGTGTAAAACATCCCACAGGACTTGCTGGAAAATTGGTTAGTAGCACAGTTGGCATCATTCA
CACAGCAGCGGGCATCGTTGTTGGCCAACTGGACCACATTTCTGCCCCAGCGGTTCTGGGTGATCTTGGCTCTGTCT
GGGTTCTCCTTCATAGCGCGCTGCCCGTTTTCGCTCGCCACATCCATCTCGATAATGTGGTCCTTCTGGATCATAAT
AGTGCCATGCAGGCATTTCACCTTGCCTTCGTAATCGGTGCATCCATGAGCCCACAGAGCGCACCCGGTGCACTCCC
AATTATTGTGGGCGATCTCAGAATAAGAATGCACCAATCCCTGCATGAATCTTCCCATCATCGCTGTCAGGGTCTTC
ATGCTACTAAATGTCAGCGGGATGCCACGGTGCTCCTCGTTCACATACTGGTGGCAGATACGCTTGTACTGCTCGTG
CTGCTCTGGCATCAGCTTGAAAGAGGTTCTCAGGTCATTATCCAGCCTATACCTCTCCATTAGCACAGCCATCACTT
CCATGCCCTTCTCCCAGGCAGATACCAGGGGCAAGCTCAAAGGATTCCTAACAGCAATAGAAGTAGCTCCTTTAGCT
ATAGGGTCATTCTTGTCGATCTTCTCAACACTTCTCTTGCCATCCTTCTCAATGATGCGCACCGGGGGGTAGCTGAA
GCCCACGGCCACCAACTGAGCCTGTTCTCTTTCTTCTTCGCTGTCCTGGCTGATGTCTTGCAGAGGGACATGCTTGG
TCTTCCTGGGCTTCTTCTTGGGAGGGATCGGGGGAGGACTGTTGCTCCGTTCCGGAGACAGGGATGACCGCGAAGTT
TCGCTTACCAGTACCACCTGGCTCTCGATAGAAGAATCGGACCCCACGCGACGGTAGGTGTTCCTCTTCGGGGGCAG
AGGTGGAGGCGACTGAGATGGGCTGCGGTCCGGCCTTGGAGGCGGATGGCTGGCAGAGCCCATTCCGCGTTCGGGGG
TGTGCTCCCGTTGGCGGTCGCTTGACTGATTTCCTCCGCGGCTGGCCATTGTGTTCTCCTAGGCAGAGAAACAACAG
ACATGGAAACTCAGCCATCACTGCCAACATCGCTGCAAGCGCCATCACACCTCGCCCCCAGCAGCGACGAGGAGGAG
AGCTTAACCACCCCACCACCCAGTCCCGCTACCACCACCTCTACCCTCGATGATGAGGAGGAGGTCGACGCAGCCCA
GGAGATGCAGGCGCAGGATAATGTGAAAGCGGAAGAGATTGAGGCAGATGTCGAGCAGGACCCGGGCTATGTGACAC
CGGCGGAGCACGAGGAGGAGCTGAAACGTTTTCTAGACAGAGAGGATGACGACCGCCCAGAGCATCACCAGGAGGCT
GGCCTCGGGGATCATGTTGCCGACTACCTCTCCGGGCTTGGGGGGGAGGACGTGCTCCTCAAACATCTAGCAAGGCA
GTCGATCATAGTTAAAGACGCACTACTCAACCTCACCGAAGTGCCCATCAGTGTGGAAGAGCTTAGCCGCGCCTACG
AGCTGAACCTCTTTTCGCCTCAGATACCCCCCAAGCGGCAGCGAAACGGCACCTGCGAGGCCAACCCTCGACTCAAC
TTCTATCCAGCTTTTACTGTCCCCGAAGTGCTGGCCACCTACCACATCTTTTTTAAGAACCAAAAGATTCCAGTCTC
CTGCCGCGCCAACCGCACCCGCGCAGATGCCCTTCTCAACTTGGGTCCGGGAGCTCGTTTACCTGATATAGCTTCCT
TGGAAGAGGTTCCAAAGATCTTTGAGGGTCTGGGAAGTGATGAGACTCGGGCCGCAAATGCTCTGCAACAGGGAGAG
AATGGCATGGATGAACATCACAGCGCTCTAGTGGAACTGGAGGGTGACAATGCCCGGCTTGCAGTGCTCAAGCGCAG
TATCGTGGTCACCCATTTTGCCTACCCCGCTGTTAACCTGCCGCCCAAAGTCATGAGCGCTGTCATGGACCATCTGC
TCATCAAACGAGCAAGTCCACTTTCAGAAAACCAGAACATGCAGGATCCAGACGCCTCGGACGAGGGCAAGCCGGTA
GTCAGTGACGAGCAGCTATCTCGCTGGCTGGGTACCAACTCCCCCCGAGATTTGGAAGAAAGACGCAAGCTTATGAT
GGCTGTAGTGCTAGTAACTGTTGAGTTGGAGTGTCTGCGCCGCTTTTTTACCGACCCCGAGACCCTGCGCAAGCTAG
AGGAGAACCTGCACTACACCTTCAGACATGGCTTCGTGCGCCAGGCATGCAAGATCTCCAACGTGGAGCTCACCAAC
CTGGTTTCATACATGGGCATTTTGCATGAGAACCGGCTAGGGCAGAGCGTTCTGCACACCACCCTGAAGGGGGAGGC
CCGCCGCGACTACATCCGAGACTGTGTCTACCTCTACCTCTGCCATACCTGGCAGACTGGTATGGGTGTGTGGCAAC
AGTGTTTGGAAGAGCAGAACCTTAAAGAGCTGGACAAGCTCTTGCAGAGATCCCTCAAAGCCCTGTGGACAGGTTTT
GACGAGCGCACCGTCGCCTCGGACCTGGCGGACATCATCTTCCCCGAGCGTCTTAGGGTTACTCTGCGAAACGGCCT
GCCAGACTTCATGAGCCAGAGCATGCTTAACAACTTTCGCTCTTTCATCCTGGAACGCTCCGGTATCCTGCCTGCCA
CCTGCTGTGCGCTGCCCTCCGACTTTGTGCCTCTCACCTACCGCGAGTGCCCACCGCCGCTATGGAGCCACTGCTAC
CTATTCCGCCTGGCCAACTACCTCTCCTACCACTCGGATGTGATAGAGGATGTGAGCGGAGACGGCCTGCTGGAATG
CCACTGCCGATGCAATTTATGCACACCCCACCGCTCCCTCGCCTGCAACCCCCAGTTGCTAAGCGAGACCCAGATCA
TCGGCACCTTCGAGTTGCAGGGTCCCAACAGTGAAGGCGAGGGGTCTTCTCCGGGGCAGAGTCTGAAACTGACACCG
GGGCTGTGGACCTCCGCCTACCTGCGCAAGTTTTATCCCGAGGACTATCATCCCTATGAGATCAGGTTCTATGAGGA
CCAGTCACATCCTCCCAAAGTCGAGCTCTCAGCCTGCGTCATCACCCAGGGGGCAATTCTGGCCCAATTGCAAGCCA
TCCAAAAATCCCGCCAAGAATTTCTGCTGAAAAAGGGAAGCGGGGTCTACCTTGACCCCCAGACCGGTGAGGAGCTC
AACACAAGGTTCCCCCAGGATGTCCCATCGCCGAGGAAGCAAGAAGCTGAAGGTGCAGCTGTCACCCCCAGAGGATA
TGGAGGAAGACTGGGACAGTCAGGCAGAGGAGGAGATGGAAGATTGGGACAGCCAGGCAGAGGAGGTGGACAGCCTG
GAGGAAGACAGTTTGGAGGAGGAAGACGAGGAGGCAGAGGAGGTGGAAGAAGCAACCGCCGCCAAACAGTTGTCATC
GGCGGCGGAGACAAGCAAGTCCCCAGACAGCAGCACGGCTACCATCTCCGCTCCGGGTCGGGGGGCCCAGCGGCGGC
CCAACAGTAGATGGGACGAGACCGGGCGATTTCCAAACCCGACCACCGCTTCCAAGACCGGTAAGAAGGAGCGACAG
GGATACAAGTCCTGGCGTGGACATAAAAACGCTATCATCTCCTGCTTGCATGAATGCGGGGGCAACATATCCTTCAC
CCGGCGATACCTGCTTTTCCACCACGGTGTGAACTTCCCCCGCAATATCTTGCATTACTACCGTCACCTCCACAGCC
CCTACTGCAGTCAGCAAGTCCCGGCAACCCCGACAGAAAAAGACAGCAGCGACAACGGTGACCAGAAAACCAGCAGT
TAGAAAATCCACAACAAGTGCAGCAGGAGGAGGACTGAGGATCACAGCGAACGAGCCAGCGCAGACCAGAGAGCTGA
GGAACCGGATCTTTCCAACCCTCTATGCCATCTTCCAGCAGAGTCGGGGGCAAGAGCAGGAATTGAAAGTAAAAAAC
CGATCTCTGCGCTCGCTCACCAGAAGTTGTTTGTATCACAAGAGCGAAGACCAACTTCAGCGCACTCTCGAGGACGC
CGAGGCTCTCTTCAACAAGTACTGCGCGCTGACTCTTAAAGAGTAGCCCTTGCCCGCGCTCATTCGAAAACGGCGGG
AATCACGTCACCCTTGGCAGCTGTCCTTTGCCCTCGTCATGAGTAAAGACATTCCCACGCCTTACATGTGGAGCTAT
CAGCCCCAAATGGGGTTGGCAGCAGGTGCTTCCCAGGACTACTCCACCCGCATGAATTGGCTTAGCGCCGGGCCCTC
AATGATATCACGGGTTAATGATATACGAGCTTATCGAAACCAGTTACTCCTAGAACAGTCAGCTCTTACCACCACAC
CCCGCCAACACCTTAATCCCCGAAATTGGCCCGCCGCCCTGGTGTACCAGGAAAATCCCGCTCCCACCACCGTACTA
CTTCCTCGAGACGCCCAGGCCGAAGTTCAGATGACTAACGCAGGTGTACAGCTGGCGGGCGGTTCCGCCCTATGTCG
TCACCGGCCTCAACAGAGTATAAAACGCCTGGTGATCAGAGGCCGAGGTATCCAGCTCAACGACGAGTCGGTTAGCT
CTTCGCTTGGTCTGCGACCAGACGGAGTCTTCCAGATCGCCGGCTGTGGGAGATCTTCCTTCACTCCTCGTCAGGCT
GTGCTGACTTTGGAGAGTTCGTCCTCGCAGCCCCGCTCGGGCGGCATCGGAACTCTCCAGTTTGTGGAGGAGTTTAC
TCCCTCTGTCTACTTCAACCCCTTCTCCGGCTCTCCTGGCCAGTACCCGGACGAGTTCATACCGAACTTCGACGCAA
TCAGCGAGTCAGTGGATGGCTATGATTGATGTCTAATGGTGGCGCGGCTGAGCTAGCTCGACTGCGACACCTAGACC
ACTGCCGCCGCTTTCGCTGTTTCGCCCGGGAACTCACCGAGTTCATTTACTTCGAACTCTCCGAGGAGCACCCTCAG
GGTCCGGCCCACGGAGTGCGGATTACCATCGAAGGGGGAATAGACTCTCGCCTGCATCGCATCTTCTCCCAGCGGCC
CGTGCTGATTGAGCGCGACCAGGGAAATACAACCATCTCCATCTACTGCATCTGTAACCACCCCGGATTGCATGAAA
GCCTTTGCTGTCTTGTTTGTGCTGAGTTTAATAAAAACTGAGTTAAGACCCTCCTACGGACTACCGCTTCTTCAATC
AGGACTTTACAACACCAACCAGATCTTCCAGAAGACCCAGACCCTTCCTCCTCTGATCCAGGACTCTAACTCTACCT
TACCAGCACCCTCCACTACTAACCTTCCCGAAACTAACAAGCTTGGATCTCATCTGCAACACCGCCTTTCACGAAGC
CTTCTTTCTGCCAATACTACCACTCCCAAAACCGGAGGTGAGCTCCGCGGTCTTCCTACTGACGACCCCTGGGTGGT
AGCGGGTTTTGTAACGTTAGGATTAGTTGCGGGTGGGCTTGTGCTAATCCTTTGCTACCTATACACACCTTGCTGTG
CATATTTAGTCATATTGTGCTGTTGGTTTAAGAAATGGGGGCCATACTAGTCGTGCTTGCTTTACTTTCGCTTTTGG
GTCTGGGCTCTGCTAATCTCAATCCTCTTGATCACGATCCATGTCTAGACTTCGACCCAGAAAATTGCACACTTACT
TTTGCACCCGACACAAGCCGTCTCTGTGGAGTTCTTATTAAGTGCGGATGGGACTGCAGGTCCGTTGAAATTACACA
TAATAACAAAACATGGAACAATACATTATCCACCACATGGGAACCAGGAGTTCCCGAGTGGTATACTGTCTCTGTCC
GAGGTCCTGACGGTTCCATTCGCATTAGTAACAACACTTTCATTTTTTCTGAAATGTGCGATCTGGCCATGTTTATG
AGCAAACAGTATGACCTATGGCCTCCTAGCAAAGAGAACATTGTGGCATTTTCCATTGCTTATTGCTTGGTAACATG
CATCATCACTGCTATCATTTGTGTGTGCATACACTTGCTTATAGTTATTCGCCCTAGACAAAGCAATGAGGAAAAAG
AGAAAATGCCTTAACCTTTTTCCTCATACCTTTTCTTTACAGCATGGCTTCTGTTACAGCTCTAATTATTGCCAGCA
TTGTCACTGTCGCTCACGGGCAAACAATTGTCCATATTACCTTAGGACATAATCACACTCTTGTAGGGCCCCCAATT
ACTTCAGAGGTTATTTGGACCAAACTTGGAAGTGTTGATTATTTTGATATAATTTGCAACAAAACTGAACCAATATT
TGTAATCTGTAACAGACAAAATCTCACGTTAATTAATGTTAGCAAAATTTATAACGGTTACTATTATGGTTATGATA
GATCCAGTAGTCAATATAAAAATTACTTAGTTCGCATAACTCAGCCCAAATCAACAGTGCCAACTATGACAATAATT
AAAATGGCTAATAAAGCATTAGAAAATTTTACATTACCAACAACGCCCAATGAAAAAAACATTCCAAATTCAATGAT
TGCAATTATTGCGGCGGTGGCATTGGGAATGGCACTAATAATAATATGCATGTTCCTATATGCTTGTTGCTATAAAA
AGTTTCAACATAAACAGGATCCACTACTAAATTTTAACATTTAATTTTTTATACAGATGATTTCCACTACAATTTTT
ATCATTACTAGCCTTGCAGCTGTAACTTATGGCCGTTCACACCTAACTGTACCTGTTGGCTCAACATGTACACTACA
AGGACCCCAAGAAGGCTATGTCACTTGGTGGAGAATATATGATAATGGAGGGTTCGCTAGACCATGTGATCAGCCTG
GTACAAAATTTTCATGCAACGGAAGAGACTTGACCATTATTAACATAACATTAAATGAGCAAGGCTTCTATTATGGA
ACCAACTATAAAAATAGTTTAGATTACAACATTATTGTAGTGCCAGCCACCACTTCTGCTCCCCGCAAATCCACTTT
CTCTAGCAGCAGTGCCAAAGCAAGCACAATTCCTAAAACAGCTTCTGCTATGTTAAAGCTTCGAAAAATCGCTTTAA
GTAATTCCACAGCAGCTCCCAATACAATTCCTAAATCAACAATTGGCATCATTACTGCCGTGGTAGTGGGATTAATG
ATTATATTTTTGTGCATAATGTACTACGCCTGCTGCTATAGAAAACATGAACAAAAAGGTGATGCATTACTAAATTT
TGATATTGTTTCAATCAAATGCCACTAACACTCTCAATGTGCAGACTACTTTAAAACATGACATGGAAAACCACACT
ACCTCCTATGCATACACAAATATTCAGCCTAAATACGCTATGCAACTTAGAAATCACCATACTAATTGTAATTGGAA
TTCTTACACTATCTGTTATTCTTTATTTTATATTCTGCCGTCAAATACCCAATGTTCATAGAAATTCTAAAAGACGT
CCCATCTATTCTCCTATGATTAGTCGTCCCCATATGGCTCTGAATGAAATCTAAGATCTTTTTTTTTCTTTTACAGT
ATGGTGAACATCAATCATGATTCCTAGAAATTTCTTCTTCACCATACTCATCTGTGCTTTCAATGTCTGTGCTACTT
TCACAGCAGTAGCCACTGCAAGCCCAGACTGTATAGGACCATTTGCTTCCTATGCACTTTTTGCCTTTGTTACTTGC
ATCTGCGTGTGTAGCATAGTCTGCCTGGTTATTAATTTTTTCCAACTGGTAGACTGGATCTTTGTGCGAATTGCCTA
CCTACGTCACCATCCCGAATACCGCAATCAAAATGTTGCGGCACTTCTTAGGCTTATTTAAAACCATGCAGGCTATG
CTACCAGTTATTTTAATTCTGCTACTACCCTGCATTGCCCTACCTTCCACCGCCACTCGCGCTACACCTGAACAACT
TAGAAAATGCAAATTTCAACAACCATGGTCATTTCTTGATTGCTACCATGAAAAATCTGATTTTCCCACATACTGGA
TAGTGATTGTTGGAATAATTAACATACTTTCATGTACCGTTTTCTCAATCACAATATACCCCACATTTAATTTTGGG
TGGAATTCTCCCAATGCACTGGGTTACCCACAAGAACTAGATGAACATATCCCACTACAACACATACAACAACCACT
AGCATTGGTAGAGTATGAAAATGAGCCACAACCTTCACTGCCTCCTGCTATTAGTTACTTCAACCTAACCGGCGGAG
ATGACTGAAATACTCACCACCTCCAATTCCGCCGAGGATCTGCTTGATATGGACGGCCGCGCCTCAGAACAGCGACT
CGCCCAACTACGCATCCGCCAGCAGCAGGAACGCGTGACCAAAGAGCTCAGAGATGTCATCCAAATTCACCAATGCA
AAAAAGGCATATTTTGTTTGGTAAAACAAGCCAAGATATCCTACGAGATCACCGCTACTGACCATCGCCTTTCTTAC
GAACTTGGCCCCCAACGACAAAAATTTACATGCATGGTGGGAATCAACCCTATAGTTATCACCCAGCAAAGTGGAGA
TACTAAGGGTTGCATTCACTGCTCTTGCGATTCCACCGAGTGCACCTACACCCTGCTGAAGACCCTATGCGGCCTAA
GAGACCTGCTACCCATGAATTAAAAATTAATAAAAAATTACTTACTTGAAATCAGCAATAAGGTCTCTGTTGAAATT
TTTTCCCAGCAGCACCTCGCTTCCCTCTTCCCAACTCTGGTATTCTAAACCCCGTTCAGCGGCATACTTTCTCCATA
CTTTAAATGGGATGTCAAATTTTAGCTCCTCTCCTGTACCCACGATCTTCATGTCTTTCTTCCCAGATGACCAAGAG
AGTCCGGCTCAGTGATTCCTTCAACCCTGTCTACCCCTATGAAGATGAAAGCACCTCCCAACACCCCTTTATAAACC
CAGGGTTTATTTCCCCAAATGGCTTTACACAAAGCCCAGACGGAGTTCTTACTTTAAATTGTTTAACCCCACTAACA
ACCACAGGCGGGCCTTTACAGTTAAAAGTGGGAGGGGGACTTATAGTGGATGACACTGATGGGACCTTACAAGAAAA
CATACGTGCTACAGCACCCATTACTAAAAATAATCATTCTGTAGAACTATCCATTGGAAATGGATTAGAAACACAAA
ACAATAAACTATGTGCCAAATTGGGAAATGGGTTAAAATTTAACAACGGTGACATTTGTATAAAGGATAGTATTAAC
ACCTTATGGACTGGAATAAAGCCTCCACCTAACTGTCAAATTGTGGAAAACACTGATACAAACGATGGCAAACTTAC
TTTAGTATTAGTAAAAAACGGAGGGCTTGTTAATGGCTACGTATCTCTAGTTGGTGTATCAGACACTGTGAACCAAA
TGTTCACACAAAAGTCAGCAACCATACAATTAAGATTATATTTCGACTCTTCTGGAAATCTATTAACTGATGAATCA
AACTTAAAAATTCCACTTAAAAATAAATCTTCTACAGCAACCAGTGAAGCTGCAACCAGCAGCAAAGCCTTTATGCC
AAGTACTACAGCTTATCCCTTTAACACCACTACTAGGGATAGTGAAAACTATATTCATGGAATATGTTACTATATGA
CTAGTTATGATAGAAGTCTAGTTCCCTTAAACATTTCTATAATGCTAAACAGCCGTACGATTTCTTCCAATGTTGCC
TATGCCATACAATTTGAATGGAATCTAAATGCAAAAGAATCTCCAGAAAGCAACATAGCTACGCTGACCACATCCCC
CTTTTTCTTTTCTTATATTAGAGAAGACGACAACTAAAAAATAAAGTTTAAGTGTTTTTATTTAAAAATCACAAAAT
TCGAGTAGTTATTTTGCCTCCCCCTTCCCATTTAACAGAATACACCAATCTCTCCCCACGCACAGCTTTAAACATTT
GGATACCATTAGAGATAGACATAGTTTTAGTTTCCACATTCCAAACAGTTTCAGAGCGAGCCAATCTGGGGTCAGTG
ATACATAAAAATGCATCGGGATAGTCTTTTAAAGCGCTTTCACAGTCCAACTGCTGCGGATGCGACTCCGGAGTCTG
GATCACAGTCATCTGGAAGAAGAACGATGGGAATCATAATCCGAAAACGGAATCGGGCGATTGTGTCTCATCAAACC
CACAAGCAGCCGCTGTCTGCGTCGCTCCGTGCGACTGCTGTTTATGGGATCGGGGTCTGCAGTGTCCTGAAGCATGA
TTTTAATAGCCCTTAACATTAACTTTCTGGTGCGATGCGCGCAGCAACGCATTCTGATTTCACTTAGATTACTACAG
TATGTACAGCACATTATCACAATATTGTTTAATAAACCATAATTAAAAGCGCTCCAGCCAAAACTCATATCTGATAC
AATCGCCCCTGCATGACCATCATACCAAATTTTAATATAAATTAAATGTCGTTCCCTCAAAAACACACTACCCACAT
ACATGATCTCTTTTGGCATGTGCATATTAACAATCTGTCTGTACCATGGACAACGTTGGTTAATCATGCAACCCAAT
ATAACCTTCCGGAACCACACTGCCAACACCGCTCCCCCAGCCATGCATTGAAGTGAACCCTGCTGATTACAATGACA
ATGAAGAACCCAATTCTCTCGACCATGAATCACTTGAGACTGAAAAATATCTATAGTAGCACAACAAAGACATAAAT
GCATGCATCTTCTCATAATTTTTAACTCATCTGGATTTAAAAACATATCCCAAGGAATGGGAAACTCTTGCAAAACA
GTAAAGCTGGCAGAACAAGGAAGACCACGAACACAACTTACACTATGCATAGTCATAGTATCACAATCTGGCAACAG
CGGGTGGTCTTCAGTCATAGAAGCTCGGGTTTCATTTTCCTCACATCGTGGTAACTGGGCTCTGGTGTAAGGGTGAT
GTCTGGCGCATGATGTCGAGCGTGCGCGCAACCTTGTCATAATGGAGTTGTTTCCTGACATTCTCGTATTTTGTATA
GCAAAATGCGGCCCTGGCACAACACACTCTTCTTCGTCTTCTATCCTGCCGCTTAGTGTGTTCCGTCTGATAATTCA
AGTACAGCCACACTCTTAAGTTGGTCAAAAGAATGCTGGCTTCAGTTGTAATCAAAACTCCATCATATTTAATTGTT
CTAAGGAAATCATCCACGGTAGCATATGCAAATCCCAACCAAGCAATGCAACTGGATTGTGTTTCAAGCAGCAGAGG
AGAGGGAAGAGACGGAAGAATCATGTTAATTTTTATTCCAAACGATCTCGCAGTACTTCAAATTGTAGATCGCGCAG
ATGGCATCTATCGCCCCCACTGTGTTGGTGAAAAAGCACAGCTAAATCAAAAGAAATGCGATTTTCAAGGTGCTCAA
CGGTGGCTTCCAACAAAGCCTCCACGCGCACATCCAAAAACAAAAGAATACCAAAAGAAGGAGCATTTTCTAACTCC
TCAAACATCATATTACATTCCTGCACCATTCCCAGATAATTTTCAGCTTTCCAGCCTTGAATTATTCGTGTCAGTTC
TTGTGGTAAATCCAAACCACACATTACAAACAGGTCACGGAGGGCGCCCTCCACCACCATTCTTAAACACACCCTCA
TAATGACAAAATATCTTGCTCCTGTGTCACCTGTAGCAAATTAAGAATGGCATCATCAATTGACATGCCCTTGGCTC
TAAGTTCTTCTCTAAGTTCTAGTTGTAAATACTCTCTCATATTATCACCAAACTGCTTAGCCAAAAGCCCCCCGGGA
ACAATAGCAGGGGACGCTACAGTGCAGTACAAGCGCAGACCTCCCCAATTGGCTCCAGCAAAAACAAGATTAGAATA
AGCATACTGGGAACCACCAGTAATATCATCAAAGTTGCTGGAAATATAATCAGGCAGAGTTTCTTGTAAAAATTGAA
TAAAAGAAAAATTTTCCAAAGAAACATTCAAAATCTCTGGGATGCAAATGCAATAGGTTACCGCGCTGCGCTCCAAC
ATTGTTAGTTTTGAATTAGTCTGCAAAATAAAAGAAACAAGCGTCATATCATAGTAGCCTGTCGAACAGGTGGATAA
ATCAGTCTTTCCATCACAAGACAAGCCACAGGGTCTCCAGCTCGACCCTCGTAAAACCTGTCATCGTGATTAAACAA
CAGCACCGAAAGTTCCTCGCGGTGGCCAGCATGAATAATTCTTGATGAAGCATATAATCCAGACATGTTAGCATCAG
TTAAAGAGAAAAAACAGCCAACATAGCCTCTGGGTATAATTATGCTTAATCTTAAGTATAGCAAAGCCACCCCTCGC
GGATACAAAGTAAAAGGCACAGGAGAATAAAAAATATAATTATTTCTCTGCCGCTGTTCAGGCAACGTCGCCCCCGG
TCCATCTAAATACACATACAAAGCCTCATCAGCCATGGCTTACCAGACAAAGAACAGCGGGCGCACAAAGCACAAGC
TCTAAAGAAGCTCTAAAGACACTCTCCAACCTCTCCACAATATATACACAAGCCCTAAACTGACGTAATGGGAGTAA
AGTATAAAAAATCCCGCCAAGCCCAACACACACCCCGAAACTGCGTCAGCAGGGAAAAATACAGTTTCACTTCCGCA
TTCCCAACAAGCGTAAGTTCCTCTTTCTCATGGTACGTCACATCCGATTAACTTGCAACGTCATTTTCCCACGGTCG
CACCGCCCCTTTTAGCCGTTCACCCCGCAGCCAATCACCACACAGCGCGCACTTTTTTAAATTACCTCATTTGCATA
TTGGCACCATTCCATCTATAAGGTATATTATATAGATAG

GenBank Accession No. AAW33370 (SEQ ID NO: 229)

MTKRVRLSDSFNPVYPYEDESTSQHPFINPGFISPNGFTQSPDGVLTLNCLTPLTTTGGPLQLKVGGGLIVDDTDGT
LQENIRATAPITKNNHSVELSIGNGLETQNNKLCAKLGNGLKFNNGDICIKDSINTLWTGIKPPPNCQIVENTDIND
GKLTLVLVKNGGLVNGYVSLVGVSDTVNQMFTQKSATIQLRLYFDSSGNLLTDESNLKIPLKNKSSTATSEAATSSK
AFMPSTTAYPFNTTTRDSENYIHGICYYMTSYDRSLVPLNISIMLNSRTISSNVAYAIQFEWNLNAKESPESNIATL
TTSPFFFSYIREDDN

GenBank Accession No. AAW33349 (SEQ ID NO: 230)

MMRRTVLGGAVVYPEGPPPSYESVMQQAAAATMQPPLEAPFVPPRYLAPTEGRNSIRYSELAPLYDTTRLYLVDNKS
ADIASLNYQNDHSNFLTTVVQNNDFTPTEASTQTINFDERSRWGGQLKTIMHTNMPNVNEYMFSNKFKARVMVSRKA
PEGVTVDDNYDHKQDILEYEWFEFTLPEGNFSATMTIDLMNNAIIDNYLEVGRQNGVLESDIGVKFDTRNFRLGWDP
ETKLIMPGVYTYEAFHPDIVLLPGCGVDFTESRLSNLLGIRKRHPFQEGFKILYEDLEGGNIPALLDVEAYENSKKE
QEAKTEAAKAAAIAKANIVVSDPVRVANAEEVRGDNYTASSVATDESLLAAVAETTETKLTIKPVEKDSKSRSYNVL
EDKVNTAYRSWYLSYNYGDPEKGVRSWTLLTTSDVTCGAEQVYWSLPDMMQDPVTFRSTRQVSNYPVVGAELMPVFS
KSFYNEQAVYSQQLRQSTSLTHVFNRFPENQILIRPPAPTITTVSENVPALTDHGTLPLRSSIRGVQRVTVTDARRR
TCPYVYKALGIVAPRVLSSRTF

GenBank Accession No. AAW33354 (SEQ ID NO: 231)

MATPSMLPQWAYMHIAGQDASEYLSPGLVQFARATDTYFNLGNKFRNPTVAPTHDVTTDRSQRLMLRFVPVDREDNT
YAYKVRYTLAVGDNRVLDMASTFFDIRGVLDRGPSFKPYSGTAYNSLAPKGAPNTSQWIAEGVKKEDGGSDEEEEKN
LTTYTFGNAPVKAEGGDITKDKGLPIGSEITDGEAKPIYADKLYQPEPQVGDETWTDTDGTTEKYGGRALKPETKMK
PCYGSFAKPTNVKGGQAKQKTTEQPQNQQVEYDIDMNFFDEASQKANFSPKIVMYAENVDLETPDTHVVYKPGTSEE
SSHANLGQQSMPNRPNYIGFRDNFIGLMYYNSTGNMGVLAGQASQLNAVVDLQDRNTELSYQLLLDSLGDRTRYFSM
WNQAVDSYDPDVRIIENHGVEDELPNYCFPLDGVGVPISSYKIIEPNGQGADWKEPDINGTSEIGQGNLFAMEINLQ
ANLWRSFLYSNVALYLPDSYKYTPANVTLPTNTNTYDYMNGRVVPPSLVDTYVNIGARWSLDAMDNVNPFNHHRNAG
LRYRSMLLGNGRYVPFHIQVPQKFFAVKNLLLLPGSYTYEWNFRKDVNMVLQSSLGNDLRVDGASISFTSINLYATF
FPMAHNTASTLEAMLRNDINDQSFNDYLSAANMLYPIPANATNVPISIPSRNWAAFRGWSFTRLKTKETPSLGSGFD
PYFVYSGSIPYLDGTFYLNHTFKKVSIMFDSSVSWPGNDRLLSPNEFEIKRTVDGEGYNVAQCNMTKDWFLVQMLAN
YNIGYQGFYVPEGYKDRMYSFFRNFQPMSRQVVDEINYKDYKAVAVPYQHNNSGFVGYMAPTMRQGQAYPANYPYPL
IGTTAVTSVTQKKFLCDRIMWRIPFSSNFMSMGALTDLGQNLLYANSAHALDMTFEVDPMDEPTLLYLLFEVFDVVR
VHQPHRGVIEAVYLRTPFSAGNATT

GenBank Accession No. AY737797 (SEQ ID NO: 205)

CATCATCAATAATATACCTTATAGATGGAATGGTGCCAATATGTAAATGAGGTGATTTTAAAAATTGTGGGGTGTGT
GGTGATTGGCTGTGGGGTTAACGGCTAAACGGGGCGGCGCGGCCGTGGGAAAATGACGTTTTGTGGGGGTGGAGTTT
TTTTGCAAGTTGTCGCGGGAAATGTGACGCATAAAAAGGCTTTTTTTCTCACGGAACTACTGACTTTTCCCACGGTA
TTTAACAGGAAATGAGGTAGTTTTGACCGGATGCAAGTGAAAATTGCTGATTTGCGCGCGAAAACTGAATGAGGAAG
TGTTTTTCTGAATAATGTGGTATTTATGGCAGGGTGGAGTATTTGTTCAGGGCCAGGTAGACTTTGACCCATTACGT
GGAGGTTTCGATTACCGTGTTTTTTACCTGAATTTCCGCGTACCGTGTCAAAGTCTTCTGTTTTTACGTAGGTGTCA
GCTGATCGCTACGGTATTTATACCTCAGGGTTTGTGTCAAGAGGCCACTCTTGAGTGCCAGCGAGAAGAGTTTTCTC
CTCTGCGCCGGCAGTTTAATAATAAAAAAATGAGAGATTTGCGATTTCTGCCTCAGGAAATAATTTCTGCTGAGACT
GGAAATGAAATACTGGAGCTTGTGGTGCACGCCCTGATGGGAGACGATCCGGAGCCACCTGTGCAGCTTTTTGAGCC
TCCTACGCTTCAGGAACTGTATGATTTAGAGGTAGAGGGATCGGAGGATTCTAATGAGGAAGCTGTGAATGGCTTTT
TTACCGATTCTATGCTTTTAGCTGCTAATGAAGGATTAGAATTAGATCCGCCTTTGGACACTTTCGATACTCCAGGG
GTGATTGTGGAAAGCGGTACAGGTGTAAGAAAATTACCTGATTTGGGTTCCGTGGACTGTGATTTGCACTGCTATGA
AGACGGGTTTCCTCCGAGTGATGAGGAGGACCATGAAAAGGAGCAGTCTATGCAGACTGCAGCGGGTGAGGGAGTGA
AGGCTGCCAGTGTTGGTTTTCAGTTGGATTGCCCGGAGCTTCCTGGACATGGCTGTAAGTCTTGTGAATTTCACAGG
AAAAATACTGGAGTAAAGGAACTGTTATGTTCGCTTTGTTATATGAGAGCGCACTGCCACTTTATTTACAGTAAGTG
TGTTTAAGTTAAAATTTAAAGGAATATGCTGTTTTTCACATGTATATTGAGTGGGAGTTTTGTGCTTCTTATTATAG
GTCCTGTGTCTGATGCTGATGAGTCACCATCTCCTGATTCTACTACCTCACCTCCTGAGATTCAAGCACCTGTTCCT
GTGGACGTGCGCAAGCCCATTCCTGTGAAGCTTAAGCCTGGGAAACGTCCAGCAGTGGAAAAACTTGAGGACTTGTT
ACAGGGTGGGGACGGACCTTTGGACTTGAGTACACGGAAACGGCCAAGACAATAAGTGTTCCATATCCGTGTTTACT
TAAGGTGACGTCAATATTTGTGTGAGAGTGCAATGTAATAAAAATATGTTAACTGTTCACTGGTTTTTATTGCTTTT
TGGGCGGGGACTCAGGTATATAAGTAGAAGCAGACCTGTATGGTTAGCTCATAGGAGCTGGCTTTCATCCATGGAGG
TTTGGGCCATTTTGGAAGACCTTAGAAAGACTAGGCAACTGTTAGAGGACGCTTCGGACGGAGTCTCCGGTTTTTGG
AGATTCTGGTTCGCTAGTGAATTAGCTAGGGTAGTTTTTAGGATAAAACAGGACTATAAAGAAGAATTTGAAAAGTT
GTTGGTAGATTGCCCAGGACTTTTTGAAGCTCTTAATTTGGGCCATCAAGTTCACTTTAAAGAAAAAGTTTTATCAG
TTTTAGACTTTTCAACCCCAGGTAGAACTGCCGCTGCTGTGGCTTTTCTTACTTTTATATTAGATAAATGGATCCCG
CAGACTCATTTCAGCAGGGGATACGTTTTGGATTTCGTAGCCACAGCATTGTGGAGAACATGGAAGGTTCGCAAGAT
GAGGACAATCTTAGGTTACTGGCCAGTGCAGCCTTTGGGTGTAGCGGGAATCCTGAGGCATCCACCGGTCATGCCAG
CGGTTCTGGAGGAGGAACAGCAAGAGGACAACCCGAGAGCCGGCCTGGACCCTCCAGTGGAGGAGGCGGAGTAGCTG
ACTTGTCTCCTGAACTGCAACGGGTGCTTACTGGATCTACGTCCACTGGACGGGATAGGGGCGTTAAGAGGGAGAGG
GCATCTAGTGGTACTGATGCTAGATCTGAGTTGGCTTTAAGTTTAATGAGTCGCAGACGTCCTGAAACCATTTGGTG
GCATGAGGTCCAGAAAGAGGGAAGGGATGAAGTTTCTGTATTGCAGGAGAAATATTCACTGGAACAGGTGAAAACAT
GTTGGTTGGAGCCTGAGGATGATTGGGAGGTGGCCATTAAAAATTATGCCAAGATAGCTTTGAGGCCTGATAAACAG
TATAAGATTACTAGACGGATTAATATCCGGAATGCTTGTTACATATCTGGAAATGGGGCTGAGGTGGTAATAGATAC
TCAAGACAAGGCAGTTATTAGATGCTGCATGATGGATATGTGGCCTGGAGTAGTCGGTATGGAAGCAGTAACTTTTG
TAAATGTTAAGTTTAGGGGAGATGGTTATAATGGAATAGTGTTTATGGCCAATACCAAACTTATATTGCATGGTTGT
AGCTTTTTTGGTTTCAACAATACCTGTGTAGATGCCTGGGGACAGGTTAGTGTACGGGGATGTAGTTTCTATGCGTG
TTGGATTGCCACAGCTGGCAGAACCAAGAGTCAATTGTCTCTGAAGAAATGCATATTCCAAAGATGTAACCTGGGCA
TTCTGAATGAAGGCGAAGCAAGGGTCCGCCACTGCGCTTCTACAGATACTGGATGTTTTATTTTAATTAAGGGCAAT
GCCAGCGTAAAGCATAACATGATTTGCGGTGCTTCCGATGAGAGGCCTTATCAAATGCTCACTTGTGCCGGTGGGCA
TTGTAATATGCTGGCTACTGTGCATATTGTTTCCCATCAACGCAAAAAATGGCCTGTTTTTGATCACAATGTGTTGA
CCAAGTGTACCATGCATGCAGGTGGGCGTAGAGGAATGTTTATGCCTTACCAGTGTAACATGAATCATGTGAAAGTG
TTGTTGGAACCAGATGCCTTTTCCAGAATGAGCCTAACAGGAATCTTTGACATGAACATGCAAATCTGGAAGATCCT
GAGGTATGATGATACGAGATCGAGGGTGCGCGCATGCGAATGCGGAGGCAAGCATGCCAGGTTCCAGCCGGTGTGTG
TAGATGTGACTGAAGATCTGAGACCGGATCATTTGGTTATTGCCCGCACTGGAGCAGAGTTCGGATCCAGTGGAGAA
GAAACTGACTAAGGTGAGTATTGGGAAAACTTGGGGTGGGGTTTTCAGATGGACAGATTGAGTAAAAATTTGTTTTT
TCTGTCTTTCAGCTGTCATGAGTGGAAACGCTTCTTTTAAGGGGGGAGTCTTCAGCCCTTATCTGACAGGGCGTCTC
CCATCCTGGGCAGGAGTTCGTCAGAATGTTATGGGATCTACTGTGGATGGAAGACCCGTCCAACCCGCCAATTCTTC
AACGCTGACCTATGCTACTTTAAGTTCTTCACCTTTGGACGCAGCTGCAGCCGCCGCCGCCGCCTCTGTTGCCGCTA
ACACTGTGCTTGGAATGGGTTACTATGGAAGTATCGTGGCTAATTCCACTTCCTCTAATAACCCTTCTACCCTGACT
CAGGACAAGTTACTTGTCCTTTTGGCCCAGCTGGAGGCTTTGACCCAACGTCTGGGTGAACTTTATCAGCAGGTGGC
CGAGTTGCGAGTACAAACTGAGTCTGCTGTCGGCACGGCAAAGTCTAAATAAAAAAAAATTCCACAATCAATGAATA
AATAAACGAGCTTGTTGTTGATTTAAAATCAAGTGTTTTTATTTCATTTTTCGCGCACGGTATGCCCTAGACCACCG
ATCTCGATCATTGAGAACACGGTGGATTTTTTCCAGAATCCTATAGAGGTGGGATTGAATGTTTAGATACATGGGCA
TTAGGCCATCTTTGGGGTGGAGATAGCTCCATTGAAGGGATTCATGCTCCGGGGTAGTGTTGTAAATCACCCAGTCA
TAACAAGGTCGCAGTGCATGGTGTTGCACAATATCTTTTAGAAGTAGGCTGATTGCCACAGATAAGCCCTTGGTGTA
GGTGTTTACAAACCGGTTGAGCTGGGAGGGGTGCATTCGGGGTGAAATTATGTGCATTTTGGATTGGATTTTTAAGT
TGGCAATATTGCCGCCAAGATCTCGTCTTGGGTTCATGTTATGAAGGACCACCAAGACGGTGTATCCGGTACATTTA
GGAAATTTATCGTGTAGCTTGGATGGAAAAGCGTGGAAAAATTTGGAGACACCCTTGTGTCCTCCGAGATTTTCCAT
GCACTCATCCATGATAATAGCAATGGGGCCGTGGGCAGCAGCGCGGGCAAACACGTTCCGTGGGTCTGACACATCAT
AGTTATGTTCCTGAGTTAAATCATCATAAGCCATTTTAATGAATTTGGGGCGGAGAGTACCCGATTGGGGTATGAAT
GTTCCTTCGGGCCCCGGAGCATAGTTCCCCTCACAGATTTGCATTTCCCAAGCTTTCAGTTCCGATGGTGGAATCAT
GTCCACCTGGGGGGCTATGAAGAACACCGTTTCTGGGGCGGGGGTGATTAGTTGGGATGATAGCAAGTTTCTGAGCA
ATTGAGATTTGCCACATCCGGTGGGGCCATAAATGATTCCGATTACAGGTTGCAGGTGGTAGTTTAGGGAACGGCAA
CTGCCGTCTTCTCGAAGCAAGGGGGCCACCTCGTTCATCATTTCCCTTACATGCATATTTTCCCGCACCAAATCCAT
TAGGAGGCGCTCTCCTCCTAGTGATAGAAGTTCTTGTAGTGAGGAAAAGTTTTTCAGCGGTTTTAGACCGTCAGCCA
TGGGCATTTTGGAGAGAGTTTGCTGCAAAAGTTCTAGTCTGTTCCACAGTTCAGTGATGTGTTCTATGGCATCTCGA
TCCAGCAGACCTCCTCGTTTCGCGGGTTTGGACGGCTCCTGGAGTAGGGTATGAGACGATGGGCGTCCAGCGCTGCC
AGGGTTCGGTCCTTCCAGGGTCTCAGTGTTCGAGTCAGGGTTGTTTCCGTCACAGTGAAGGGGTGTGCGCCTGCTTG
GGCGCTTGCCAGGGTGCGCTTCAGACTCATTCTGCTGGTGGAGAACTTCTGTCGCTTGGCGCCCTGTATGTCGGCCA
AGTAGCAGTTTACCATGAGTTCGTAGTTGAGCGCCTCGGCTGCGTGGCCTTTGGCGCGGAGCTTACCTTTGGAAGTT
TTCTTGCATACCGGGCAGTATAGGCATTTCAGCGCATACAGCTTGGGCGCAAGGAAAATGGATTCTGGGGAGTATGC
ATCTGCGCCGCAGGAGGCGCAAACAGTTTCACATTCCACCAGCCAGGTTAAATCCGGTTCATTGGGGTCAAAAACAA
GTTTTCCGCCATATTTTTTGATGCGTTTCTTACCTTTGGTCTCCATGAGTTCGTGTCCTCGTTGAGTGACAAACAGG
CTGTCCGTATCCCCGTAGACTGATTTTACAGGCCTCTTCTCCAGTGGAGTGCCTCGGTCTTCTTCGTACAGGAACTC
TGACCACTCTGATACAAAGGCGCGCGTCCAGGCCAGCACAAAGGAGGCTATGTGGGAGGGGTAGCGATCGTTGTCAA
CCAGGGGGTCCACCTTTTCCAAAGTATGCAAACACATGTCACCCTCTTCAACATCCAGGAATGTGATTGGCTTGTAG
GTGTATTTCACGTGACCTGGGGTCCCCGCTGGGGGGGTATAAAAGGGGGCGGTTCTTTGCTCTTCCTCACTGTCTTC
CGGATCGCTGTCCAGGAACGTCAGCTGTTGGGGTAGGTATTCCCTCTCGAAGGCGGGCATGACCTCTGCACTCAGGT
TGTCAGTTTCTAAGAACGAGGAGGATTTGATATTGACAGTGCCGGTTGAGATGCCTTTCATGAGGTTTTCGTCCATT
TGGTCAGAAAACACAATTTTTTTATTGTCAAGTTTGGTGGCAAATGATCCATACAGGGCGTTGGATAAAAGTTTGGC
AATGGATCGCATGGTTTGGTTCTTTTCCTTGTCCGCGCGCTCTTTGGCAGCGATGTTGAGTTGGACATACTCGCGTG
CTAGGCACTTCCATTCGGGGAAGATAGTTGTCAATTCATCTGGCACGATTCTCACTTGCCACCCTCGATTATGCAAG
GTAATTAAATCCACACTGGTGGCCACCTCGCCTCGAAGGGGTTCGTTGGTCCAACAGAGCCTACCTCCTTTCCTAGA
ACAGAAAGGGGGAAGTGGGTCTAGCATAAGTTCATCGGGAGGGTCTGCATCCATGGTAAAGATTCCCGGAAGTAAAT
CCTTATCAAAATAGCTGATGGGAGTGGGGTCATCTAAGGCCATTTGCCATTCTCGAGCTGCCAGTGCACGCTCATAT
GGGTTAAGGGGACTGCCCCAGGGCATGGGATGGGTGAGTGCAGAGGCATACATGCCACAGATGTCATAGACGTAGAT
GGGATCCTCAAAGATGCCTATATAGGTTGGATAGCATCGCCCCCCTCTGATACTTGCTCGCACATAGTCATATAGTT
CATGTGATGGCGCTAGCAACCCCGGACCCAAGTTGGTGCGATTGGGTTTTTCTGTTCTGTAGACAATCTGGCGAAAG
ATGGCGTGAGAATTGGAAGAGATGGTGGGTCTTTGAAAAATGTTGAAATGGGCATGAGGTAGACCTACAGAGTCTCT
GACAAAGTGGGCATAAGATTCTTGAAGCTTGGTTACCAGTTCGGCGGTGACAAGTACGTCTAGGGCGCAGTAGTCAA
GTGTTTCTTGAATGATGTCATAACCTGGTTGGTTTTTCTTTTCCCACAGTTCGCGGTTGAGAAGGTATTCTTCGCGA
TCCTTCCAGTACTCTTCTAGCGGAAACCCGTCTTTGTCTGCACGGTAAGATCCTAGCATGTAGAACTGATTAACTGC
CTTGTAAGGGCAGCAGCCCTTCTCTACGGGTAGAGAGTATGCTTGAGCAGCTTTTCGCAGCGAAGCGTGAGTAAGGG
CGAAGGTGTCTCTGACCATGACTTTGAGAAATTGGTATTTGAAGTCCATGTCGTCACAGGCTCCCTGTTCCCAGAGT
TGGAAGTCTACCCGTTTCTTGTAGGCGGGGTTGGGCAAAGCGAAAGTAACATCGTTGAAGAGAATCTTACCGGCTCT
GGGCATAAAATTGCGAGTGATGCGGAAAGGCTGTGGTACTTCCGCTCGATTGTTGATCACCTGGGCAGCTAGGACGA
TCTCGTCGAAACCGTTGATGTTGTGTCCTACGATGTATAATTCTATGAAACGCGGCGTGCCTTTGACGTGAGGTAGC
TTATTGAGCTCATCAAAGGTTAGGTCTGTAGGGTCAGATAAGGCGTAGTGTTCGAGAGCCCATTCGTGCAGGTGAGG
ATTTGCATGTAGGAATGATGACCAAAGATCCACCGCCAGTGCTGTTTGTAACTGGTCCCGATACTGACGAAAATGCT
GGCCAATTGCCATTTTTTCTGGAGTGACACAGTAGAAGGTTCTGGGGTCTTGTTGCCATCGATCCCACTTTAGTTTA
ATGGCTAGATCGTGGGCCATGTTGACGAGACGCTCTTCTCCTGAGAGTTTCATGACCAGCATGAAAGGAACTAGTTG
TTTGCCAAAGGACCCCATCCAGGTGTAAGTTTCCACATCGTAGGTCAGGAAGAGTCTTTCTGTGCGAGGATGAGAGC
CGATCGGGAAGAACTGGATTTCCTGCCACCAGTTGGAGGATTGGCTGTTGATGTGATGGAAGTAGAAGTTTCTGCGG
CGCGCCGAGCATTCGTGTTTGTGCTTGTACAGACGGCCGCAGTAGTCGCAGCGTTGCACGGGTTGTATCTCGTGAAT
GAGCTGTACCTGGCTTCCCTTGACGAGAAATTTCAGTGGGAAGCCGAGGCCTGGCGATTGTATCTCGTGCTCTTCTA
TATTCGCTGTATCGGCCTGTTCATCTTCTGTTTCGGTGGTGGTCATGCTGACGAGCCCCCGCGGGAGGCAAGTCCAG
ACCTCGGCGCGGGAGGGGCGGAGCTGAAGGACCAGAGCGCGCAGGCTGGAGCTGTCCAGAGTCCTGAGACGCTGCGG
ACTCAGGTTAGTAGGTAGGGACAGAAGATTAACTTGCATGATCTTTTCCAGGGCGTGCGGGAGGTTCAGATGGTACT
TGATTTCCACAGGTTCGTTTGTAGAGATGTCAATGGCTTGCAGGGTTCCGTGTCCTTTGGGCGCCACTACCGTACCT
TTGTTTTTTCTTTTGATCGGTGGTGGCTCTCTTGCTTCTTGCATGCTCAGAAGCGATGACGGGGACGCGCGCCGGGC
GGAAGCGGTTGTTCCGGACCCGGAGGCATGGCTGGTAGTGGCACGTCGGCGCCGCGCACGGGCAGGTTCTGGTACTG
CGCTCTGAGAAGACTTGCGTGCGCCACCACGCGTCGATTGACGTCTTGTATCTGACGTCTCTGGGTGAAAGCTACCG
GCCCCGTGAGCTTGAACCTGAAAGAGAGTTCAACAGAATCAATTTCGGTATCGTTAACGGCAGCTTGTCTCAGTATT
TCTTGTACGTCACCAGAGTTGTCCTGGTAGGCGATCTCCGCCATGAACTGCTCGATTTCTTCCTCCTGAAGATCTCC
GCGACCCGCTCTCTCGACGGTGGCCGCGAGGTCATTGGAGATACGGCCCATGAGTTGGGAGAATGCAGTCATGCCCG
CCTCGTTCCAGACGCGGCTGTAAACCACGGCCCCCTCGGAGTCTCTTGCGCGCATCACCACCTGAGCGAGGTTAAGC
TCCACGTGTCTGGTGAAGACCGCATAGTTGCATAGGCGCTGAAAAAGGTAGTTGAGTGTGGTGGCAATGTGTTCGGC
GACGAAGAAATACATGATCCATCGTCTCAGCGGCATTTCGCTGACATCGCCCAGAGCTTCCAAGCGCTCCATGGCCT
CGTAGAAGTCCACGGCAAAATTAAAAAACTGGGAGTTTCGCGCGGACACGGTCAATTCCTCCTCGAGAAGACGGATG
AGTTCGGCTATGGTGGCCCGTACTTCGCGTTCGAAGGCTCCCGGGATCTCTTCTTCCTCTTCTATCTCTTCTTCCAC
TAACATCTCTTCTTCGTCTTCAGGCGGGGGCGGAGGGGGCACACGGCGACGTCGACGGCGCACGGGCAAACGGTCGA
TGAATCGTTCAATGACCTCTCCGCGGCGGCGGCGCATGGTTTCAGTGACGGCGCGGCCGTTCTCGCGCGGTCGCAGA
GTAAAAACACCGCCGCGCATCTCCTTAAAGTGGTGACTGGGAGGTTCTCCGTTTGGGAGGGAGAGGGCGCTGATTAT
ACATTTTATTAATTGGCCCGTAGGGACTGCGCGCAGAGATCTGATCGTGTCAAGATCCACGGGATCTGAAAACCTTT
CGACGAAAGCGTCTAACCAGTCACAGTCACAAGGTAGGCTGAGTACGGCTTCTTGTGGGCGGGGGTGGTTATGTGTT
CGGTCTGGGTCTTCTGTTTCTTCTTCATCTCGGGAAGGTGAGACGATGCTGCTGGTGATGAAATTAAAGTAGGCAGT
TCTAAGACGGCGGATGGTGGCGAGGAGCACCAGGTCTTTGGGTCCGGCTTGCTGGATACGCAGGCGATTGGCCATTC
CCCAAGCATTATCCTGACATCTAGCAAGATCTTTGTAGTAGTCTTGCATGAGCCGTTCTACGGGCACTTCTTCCTCA
CCCGTTCTGCCATGCATACGTGTGAGTCCAAACCCGCGCATTGGTTGTACCAGTGCCAAGTCAGCTACGACTCTTTC
GGCGAGGATGGCTTGCTGTACTTGGGTGAGGGTGGCTTGAAAGTCATCAAAATCCACAAAGCGGTGGTAAGCCCCGG
TATTAATGGTGTAAGCACAGTTGGCCATGACTGACCAGTTAACTGTCTGGTGACCAGGGCGCACGAGCTCGGTGTAT
TTAAGGCGCGAATAGGCGCGGGTGTCAAAGATGTAATCGTTGCAGGTGCGCACCAGATACTGGTAACCTATAAGAAA
ATGCGGCGGTGGTTGGCGGTAGAGAGGCCATCGTTCTGTAGCTGGAGCGCCGGGGGCGAGGTCTTCCAACATAAGGC
GGTGATAGCCGTAGATGTACCTGGACATCCAGGTGATTCCTGCGGCGGTAGTAGAAGCCCGAGGAAACTCGCGTACG
CGGTTCCAAATGTTGCGTAGCGGCATGAAGTAGTTCATTGTAGGCACGGTTTGACCAGTGAGGCGCGCGCAGTCATT
GATGCTCTATAGACACGGAGAAAATGAAAGCGTTCAGCGACTCGACTCCGTAGCCTGGAGGAACGTGAACGGGTTGG
GTCGCGGTGTACCCCGGTTCGAGACTTGTACTCGAGCCGGCCGGAGCCGCGGCTAACGTGGTATTGGCACTCCCGTC
TCGACCCAGCCTACAAAAATCCAGGATACGGAATCGAGTCGTTTTGCTGGTTGCCGAATGGCAGGGAAGTGAGTCCT
ATTTTTTTTTTTTGCCGCTCAGATGCATCCCGTGCTGCGACAGATGCGTCCCCAACAACAGCCCCCCTCGCAGCAGC
AGCAACCACAAAAGGCTGTCCCTGCAACTACTGCAACTGCCGCTGTGAGCGGTGCGGGACAGCCCGCCTATGATCTG
GACTTGGAAGAGGGCGAAGGACTGGCACGTCTAGGTGCGCCTTCGCCCGAGCGGCATCCGCGAGTTCAACTGAAAAA
AGATTCTCGCGAGGCGTATGTGCCCCAACAGAACCTATTTAGAGACAGAAGCGGCGAGGAGCCGGAGGAGATGCGAG
CTTCCCGCTTTAACGCGGGTCGTGAGCTGCGTCACGGTTTGGACAGAAGACGAGTGTTGCGGGACGAGGATTTCGAA
GTTGATGAAGTGACAGGGATCAGTCCTGCCAGGGCACACGTGGCTGCAGCCAACCTTGTATCGGCTTACGAACAGAC
AGTAAAGGAAGAGCGTAATTTCCAAAAGTCTTTTAATAATCATGTGCGAACCCTCATTGCCCGCGAAGAAGTCACCC
TTGGTTTGATGCATTTGTGGGATTTGATGGAAGCTATCATTCAGAACCCTACTAGCAAACCTCTGACCGCACAGCTG
TTTCTGGTGGTGCAACACAGCAGAGACAATGAGGCTTTCAGAGAGGCGCTGCTCAACATCACCGAACCCGAGGGGAG
ATGGTTGTATGATCTTATCAACATTCTACAGAGTATCATAGTGCAGGAGCGGAGCCTGGGCCTGGCCGAGAAGGTGG
CTGCCATCAATTACTCGGTTTTGAGCTTGGGAAAGTATTACGCTCGCAAGATCTACAAGACTCCATACGTTCCCATA
GACAAGGAGGTGAAGATAGATGGGTTCTACATGCGCATGACGCTGAAGGTGTTGACCCTGAGCGATGATCTTGGGGT
GTACCGCAATGACAGAATGCATCGCGCGGTGAGCGCCAGCAGGAGGCGCGAGTTAAGCGACAGGGAACTGATGCACA
GTTTGCAAAGAGCTCTAACTGGAGCTGGAACCGAGGGTGAGAATTACTTTGATATGGGAGCTGACTTGCAGTGGCAG
CCTAGTCGCAGGGCTCTGAACGCCGCGACGGCAGGATGTGAGCTTCCTTACATAGAAGAGGCGGATGAAGGCGAGGA
GGAAGAGGGCGAGTACTTGGAAGACTGATGGCACAACCCGTGTTTTTTGCTAGATGGAACAGCAAGCACCGGATCCC
GCAATGCGGGCGGCGCTGCAGAGCCAGCCGTCCGGCATTAACTCCTCGGACGATTGGACCCAGGCCATGCAACGTAT
CATGGCGTTGACGACTCGCAACCCCGAAGCCTTTAGACAGCAACCCCAGGCCAACCGTCTATCGGCCATCATGGAAG
CTGTAGTGCCTTCCCGCTCTAATCCCACTCATGAGAAGGTCCTGGCCATCGTGAACGCGTTGGTGGAGAACAAAGCT
ATTCGTCCAGATGAGGCCGGACTGGTATACAACGCTCTCTTAGAACGCGTGGCTCGCTACAACAGTAGCAATGTGCA
AACCAATTTGGACCGTATGATAACAGATGTACGCGAAGCCGTGTCTCAGCGCGAAAGGTTCCAGCGCGATGCCAACC
TGGGTTCGCTGGTGGCGTTAAATGCTTTCTTGAGTACTCAGCCTGCTAATGTGCCGCGTGGTCAACAGGATTATACT
AACTTTTTAAGTGCTTTGAGACTGATGGTATCAGAAGTACCTCAGAGCGAAGTATATCAGTCCGGTCCTGATTACTT
CTTTCAGACTAGCAGACAGGGCTTGCAGACGGTAAATCTGAGCCAAGCTTTTAAAAACCTTAAAGGTTTGTGGGGAG
TGCATGCCCCGGTAGGAGAAAGAGCAACCGTGTCTAGCTTGTTAACTCCGAACTCCCGCCTATTATTACTGTTGGTA
GCTCCTTTCACCGACAGCGGTAGCATCGACCGTAATTCCTATTTGGGTTACCTACTAAACCTGTATCGCGAAGCCAT
AGGGCAAAGTCAGGTGGACGAGCAGACCTATCAAGAAATTACCCAAGTCAGTCGCGCTTTGGGACAGGAAGACACTG
GCAGTTTGGAAGCCACTCTGAACTTCTTGCTTACCAATCGGTCTCAAAAGATCCCTCCTCAATATGCTCTTACTGCG
GAGGAGGAGAGGATCCTTAGATATGTGCAGCAGAGCGTGGGATTGTTTCTGATGCAAGAGGGGGCAACTCCGACTGC
AGCACTGGACATGACAGCGCGAAATATGGAGCCCAGCATGTATGCCAGTAACCGACCTTTCATTAACAAACTGCTGG
ACTACTTGCACAGAGCTGCCGCTATGAACTCTGATTATTTCACCAATGCCATCTTAAACCCGCACTGGCTGCCCCCA
CCTGGTTTCTACACGGGCGAATATGACATGCCCGACCCTAATGACGGATTTCTGTGGGACGACGTGGACAGCGATGT
TTTTTCACCTCTTTCTGATCATCGCACGTGGAAAAAGGAAGGCGGCGATAGAATGCATTCTTCTGCATCGCTGTCCG
GGGTCATTGGTGCTACCGCGGCTGAGCCCGAGTCTGCAAGTCCTTTTCCTAGTCTACCCTTTTCTCTACACAGTGTA
CGTAGCAGCGAAGTGGGTAGAATAAGTCGCCCGAGTTTAATGGGCGAAGAGGAGTACCTAAACGATTCCTTGCTCAG
ACCGGCAAGAGAAAAAAATTTCCCAAACAATGGAATAGAAAGTTTGGTGGATAAAATGAGTAGATGGAAGACTTATG
CTCAGGATCACAGAGACGAGCCTGGGATCATGGGGACTACAAGTAGAGCGAGCCGTAGACGCCAGCGCCATGACAGA
CAGAGGGGTCTTGTGTGGGACGATGAGGATTCGGCCGATGATAGCAGCGTATTGGACTTGGGTGGGAGAGGAAGGGG
CAACCCGTTTGCTCATTTGCGCCCTCGCTTGGGTGGTATGTTGTAAAAAAAAATAAAAAAGAAAAAACTCACCAAGG
CCATGGCGACGAGCGTACGTTCGTTCTTCTTTATTATCTGTGTCTAGTATAATGAGGCGAGTCGTGCTAGGCGGAGC
GGTGGTGTATCCGGAGGGTCCTCCTCCTTCGTACGAGAGCGTGATGCAGCAGCAGCAGGCGACGGCGGTGATGCAAT
CCCCACTGGAGGCTCCCTTTGTGCCTCCGCGATACCTGGCACCTACGGAGGGCAGAAACAGCATTCGTTACTCGGAA
CTGGCACCTCAGTACGATACCACCAGGTTGTATCTGGTGGACAACAAGTCGGCGGACATTGCTTCTCTGAACTATCA
GAATGACCACAGCAACTTCTTGACCACGGTGGTGCAAAACAATGACTTTACCCCTACGGAAGCCAGCACCCAGACCA
TTAACTTTGATGAACGATCGCGGTGGGGCGGTCAGCTAAAAACCATCATGCATACTAACATGCCCAACGTGAACGAG
TATATGTTTAGTAACAAGTTCAAAGCGCGTGTGATGGTGTCCAGAAAACCTCCTGAGGGTGTTAGAGTAGACGATAA
TTATGATCATAAGCAAGATATTCTAAAATACGAGTGGTTCGAGTTTACTTTGCCAGAAGGCAACTTTTCGGTCACTA
TGACTATCGACTTGATGAACAATGCCATCATAGACAATTACTTGAAAGTGGGCAGACAGAATGGAGTGTTGGAAAGT
GACATTGGTGTTAAGTTCGACACTAGGAACTTCAAGTTGGGATGGGATCCAGAAACTAAGTTGATCATGCCTGGGGT
TTACACCTATGAGGCCTTCCATCCTGACATCGTATTGCTGCCTGGCTGCGGAGTGGACTTTACCGAAAGCCGTCTGA
GCAACCTTCTTGGCATTAGAAAGAAACACCCATTCCAAGAGGGTTTTAAGATCTTGTATGAGGATTTAGAAGGAGGA
AATATTCCAGCCCTTTTGGATGTAGATGCTTATGAGAACAGCAAGAAAGATCAAAAAGCCAAAATAGAAGCTGCTGC
AGAAGCTAAAGCAAACATAGTTGCCAACGATCCGGTAAGGGTGGCTAACGCTAGTGAAATCAGGGGAGACAGTTTTG
CCGCAACATCCGTTCCGACTAAAGAATCATTATTGGATGATGTGTCTCAAAACATAGAGTTAAAACTCACTATTAAG
CCTGTGGAAAAAGATGGCAAAAACAGAAGTTACAATGTGTTGGAAGATAAAATCAACACGGCCTATCGCAGTTGGTA
CCTTTCGTACAATTATGGCGACCCCGAAAAAGGAGTGCGTTCCTGGACATTGCTCACCACCTCAGATGTCACCTGCG
GAGCGGAGCAGGTCTACTGGTCGCTTCCAGACATGATGCAGGATCCTGTCACTTTCCGCTCCACTAGACAAGTCAGT
AACTACCCTGTGGTGGGTGCAGAGCTTATGCCCGTCTTTTCAAAGAGCTTCTACAACGAACAAGCTGTGTACTCCCA
GCAGCTCCGCCAGTCCACCTCGCTTACGCACGTCTTCAACCGCTTTCCTGAGAACCAGATTTTAATCCGTCCGCCGG
CGCCCACAATTACCACCGTCAGTGAAAACGTTCCTGCTCTCACAGATCACGGGACCCTGCCGTTGCGCAGCAGTATC
CGGGGAGTCCAACGTGTGACCGTTACTGACGCCAGACGCCGCACCTGTCCCTACGTGTACAAGGCACTGGGCATAGT
CGCACCGCGCGTCCTTTCAAGCCGCACTTTCTAAAAAAAAAAAAAAAATGTCCGTTCTTATCTCGCCCAGTAATAAC
ACCGGTTGGGGTCTGCGCGCTCCCAGCAAGATGTACGGAGGCGCACGCAAACGTTCTACCCAACATCCCGTGCGTGT
TCGCGGGCATTTTCGCGCTCCATGGGGTGCCCTCAAGGGCCGCACTCGCGTTCGAACCACCGTCGATGATGTAATCG
ATCAGGTGGTTGCCGACGCCCGTAATTATACTCCTACTGCGCCTACATCTACTGTGGACGCAGTTATTGACAGTGTA
GTGGCTGACGCTCGCAACTATGCTCGACGTAAGAGCCGGCGAAGGCGCATTGCCAGACGTCACCGAGCTACCACTGC
CATGCGAGCAGCAAGAGCTCTGCTACGAAGAGCTAGACGCGTGGGGCGAAGAGCCATGCTTAGGGCGGCCAGACGTG
CAGCTTCGGGCGCCAGCGCCGGCAGGTCCCGCAGGCAAGCAGCCGCTGTCGCAGCGGCGACTATTGCCGACATGGCC
CAATCGCGAAGAGGCAATGTATACTGGGTGCGTGACGCTGCCACCGGTCAACGTGTACCCGTGCGCACCCGTCCCCC
TCGCACTTAGAAGATACTGAGCAGTCTCCGATGTTGTGTCCCAGCGGCGAGGATGTCCAAGCGCAAATACAAGGAAG
AAATGCTGCAGGTTATCGCACCTGAAGTCTACGGCCAACCGTTGAAGGATGAAAAAAAACCCCGCAAAATCAAGCGG
GTAAAAAAGGACAAAAAAGAAGAGGAAGATGGCGATGATGGGCTGGCGGAGTTTGTGCGCGAGTTTGCCCCACGGCG
ACGCGTGCAATGGCGTGGGCGCAAAGTTCGACATGTGTTGAGACCTGGAACTTCGGTGGTCTTTACACCCGGCGAGC
GTTCAAGCGCTACTTTTAAGCGTTCCTATGATGAGGTGTACGGGGATGATGATATTCTTGAGCAGGCAGCTGACCGA
TTAGGCGAGTTTGCTTATGGCAAGCGTAGTAGAATAAATCCCAAGGATGAAACAGTGTCCATACCCTTGGATCATGG
AAATCCCACCCCTAGTCTTAAACCGGTCACTTTGCAGCAAGTGTTACCCGTAACTCCGCGAACAGGTGTTAAACGCG
AAGGTGAAGATTTGTATCCCACTATGCAACTGATGGTGCCCAAACGCCAGAAGTTGGAGGACGTTTTGGAGAAAGTA
AAAGTGGATCCAGATATTCAACCTGAGGTTAAAGTGAGACCCATTAAGCAGGTAGCGCCTGGTCTGGGAGTACAAAC
TGTAGACATTAAAATTCCCACTGAAAGTATGGAAGTGCAAACTGAACCCGCAAAGCCTACTGCCACCTCCACTGAAG
TGCAAACGGACCCATGGATGCCCATGCCTATTACAACTGACGCCGTCGGTCCCACTCGAAGATCCCGACGAAAGTAC
GGTCCAGCAAGTCTGTTGATGCCCAACTATGTCGTACACCCATCTATTATTCCTACTCCTGGTTACCGAGGCACTCG
CTACTATCGCAGCCGAAACAGTACTTCCCGCCGTCGCCGCAAGACACCTGCAAATCGCAGTCGTCGCCGTAGACGCA
CAAGCAAACCGATTCCCGGCGCCCTGGTGCGGCAAGTGTACCGCAATGGTAGTGCGGAACCTTTGACACTGCCGCGT
GCGCGTTACCATCCTAGTATCATCACTTAATCAATGTTGCCGCTGCCTCCTTGCAGATATGGCCCTCACTTGTCGCC
TTCGCGTTCCCATCACTGGTTACCGAGGAAGAAACTCGCGCCGTAGAAGAGGGATGTTGGGGCGCGGAATGCGACGC
TACAGGCGACGGCGTGCTATCCGCAAGCAATTGCGGGGTGGTTTTTTGCCAGCCTTAATTCCAATTATCGCTGCTGC
GATTGGCGCAATACCAGGCATAGCTTCCGTGGCGGTTCAGGCCTCGCAACGACATTGACATTGGAAAAAAAAAAAAC
GTATAAATAAAAAATACAATGGACTCTGACACTCCTGGTACTGTGACTATGTTTTCTTAGAGATGGAAGACATCAAT
TTTTCATCCTTGGCTCCGCGACACGGCACGAAGCCGTACATGGGCACCTGGAGCGACATCGGCACGAGCCAACTGAA
CGGGGGCGCCTTCAATTGGAGCAGTATCTGGAGCGGGCTTAAAAATTTTGGCTCAACCATAAAAACATACGGGAACA
AAGCTTGGAACAGCAGTACAGGACAGGCGCTTAGAAATAAACTTAAAGACCAGAACTTCCAACAAAAAGTAGTCGAT
GGGATAGCTTCCGGTATCAATGGAGTGGTAGATTTGGCTAACCAGGCTGTGCAGAAAAAGATAAACAGTCGTTTGGA
CCCGCCGCCAGCAACCCCAGGTGAAATGCAAGTGGAGGAAGAAATTCCTCCGCCAGAAAAACGAGGCGACAAGCGTC
CGCGTCCCGATTTGGAAGAGACGCTGGTGACGCGCGTAGATGAACCGCCTTCTTATGAGGAAGCAACGAAGCTTGGA
ATGCCCACCACTAGACCGATAGCCCCTATGGCCACCGGGGTGATGAAACCTTCTCAGTTGCATCGACCCGTCACCTT
GGATTTGCCCCCTCCTCCTGCTGCTACTGCTGTACCCGCTTCTAAGCCTGTCGCTGCCCCGAAACCAGTCGCCGTAG
CCAGGTCACGTCCCGGGGGCGCTCCTCGTCCAAATGCACACTGGCAAAATACTCTGAACAGCATCGTGGGTCTAGGC
GTGCAAAGTGTAAAACGCCGTCGCTGCTTTTAATTAAATATGGAGTAGCGCTTAACTTGCCTATCTGTGTATATGTG
TCATTACACGCCGTCACAGCATCAGAGGAAAAAAGGAAGAGGTCGTGCGTCGACGCTGAGTTACTTTCAAGATGGCC
ACCCCATCGATGCTGCCCCAATGGGCATACATGCACATCGCCGGACAGGATGCTTCGGAGTACCTGAGTCCGGGTCT
GGTGCAGTTCGCCCGCGCCACAGACACCTACTTCAATCTGGGAAATAAGTTTAGAAATCCTACCGTAGCGCCGACCC
ACGATGTGACCACCGATCGTAGCCAGCGGCTCATGTTGCGCTTCGTGCCCGTTGACCGGGAGGACAATACATACTCT
TACAAAGTGCGGTACACCCTGGCCGTGGGCGACAACAGAGTGCTGGATATGGCCAGCACGTTCTTTGACATTAGGGG
CGTGTTGGACAGAGGTCCCAGTTTTAAACCCTATTCTGGTACGGCTTACAACTCCCTGGCTCCTAAAGGCGCTCCAA
ATGCATCTCAGTGGTTGGATAAGGGAGTTACAAGCACTGGCCTAGTGGACGACGGCAATACTGATGATGGGGAAGAA
GCCAAAAAAGCAACATACACTTTTGGTAATGCTCCAGTAAAAGCCGAGGCTGAAATCACAAAAGACGGATTGCCGGT
GGGCTTGGAAGTTTCAACTGAAGGTCCTAAACCAATCTATGCTGATAAGCTTTATCAGCCAGAACCTCAAGTGGGAG
ACGAAACTTGGACTGACCTAGACGGAAAAACCGAAGAGTATGGAGGGAGGGTTCTTAAACCTGAAACTAAAATGAAA
CCCTGCTACGGATCTTTTGCTAAACCTACTAATATTAAAGGAGGTCAGGCAAAGGTAAAACCAAAAGAAGACGATGG
CACTAACAACATCGAGTATGACATTGACATGAACTTCTTTGACTTAAGATCACAAAGATCAGAACTCAAACCTAAAA
TTGTAATGTATGCAGAAAATGTGGACCTGGAATGTCCAGATACTCATGTTGTGTACAAACCTGGAGTTTCAGATGCT
AGTTCTGAGACCAATCTTGGACAACAGTCTATGCCCAACAGACCCAACTACATTGGCTTCAGAGATAACTTCATCGG
ACTTATGTACTATAACAGTACTGGCAACATGGGGGTACTGGCTGGCCAAGCGTCTCAGTTGAATGCAGTGGTTGACT
TGCAGGACAGAAACACAGAACTGTCTTACCAACTCTTGCTTGACTCTCTGGGCGACAGAACCAGATACTTTAGCATG
TGGAATCAGGCTGTGGACAGTTATGATCCTGATGTACGTGTTATTGAAAATCATGGTGTGGAAGATGAACTTCCCAA
CTATTGTTTTCCGTTGGATGGTGTCGGTCCGCGAACAGATAGTTACAAGGAGATTAAGCCAAATGGAGACCAATCTA
CTTGGACAAATGTAGACCCAACTGGCAGCAGTGAACTTGCTAAGGGAAATCCATTTGCCATGGAAATTAACCTTCAA
GCCAATCTATGGCGAAGTTTCCTTTATTCCAATGTGGCTCTATATCTCCCAGACTCGTACAAATACACCCCGTCCAA
TGTCACTCTTCCAGAAAACAAAAACACCTACGACTACATGAACGGGCGGGTGGTGCCGCCATCTCTAGTAGACACCT
ATGTGAACATTGGTGCCAGGTGGTCTCTGGATGCCATGGACAATGTCAACCCATTCAACCACCACCGTAACGCTGGC
TTGCGTTACCGATCCATGCTTCTGGGTAACGGACGTTATGTGCCTTTCCACATACAAGTGCCTCAAAAATTCTTCGC
TGTTAAAAACCTGCTGCTTCTCCCAGGCTCCTACACTTATGAGTGGAACTTTAGGAAGGATGTAAACATGGTTCTAC
AGAGTTCCCTCGGTAACGACCTACGGGTAGATGGCGCCAGCATCAGTTTTACGAGCATCAACCTCTATGCTACTTTT
TTCCCCATGGCTCACAACACCGCTTCCACCCTTGAAGCCATGCTGCGGAATGACACCAATGATCAGTCATTCAACGA
CTACCTATCTGCAGCTAACATGCTCTACCCCATTCCTGCCAATGCAACCAATATTCCCATTTCCATTCCTTCTCGCA
ACTGGGCGGCTTTCAGAGGCTGGTCATTTACCAGACTGAAAACCAAAGAAACTCCCTCTTTGGGGTCTGGATTTGAC
CCCTACTTCGTCTATTCTGGTTCTATTCCCTACCTGGATGGTACCTTCTACCTGAACCACACTTTTAAGAAGGTTTC
CATCATGTTTGACTCTTCAGTGAGCTGGCCTGGAAATGACAGGTTACTATCTCCTAACGAATTTGAAATAAAGCGCA
CTGTGGATGGCGAAGGCTACAACGTAGCCCAATGCAACATGACCAAAGACTGGTTCTTGGTACAGATGCTCGCCAAC
TACAACATCGGCTATCAGGGCTTCTACATTCCAGAAGGATACAAAGATCGCATGTATTCATTTTTCAGAAACTTCCA
GCCCATGAGCAGGCAGGTGGTTGATGAGGTCAATTACAAAGACTTCAAGGCCGTCGCCATACCCTACCAACACAACA
ACTCTGGCTTTGTGGGTTACATGGCTCCGACCATGCGTCAAGGTCAACCCTATCCCGCTAACTATCCCTATCCACTC
ATTGGAACAACTGCCGTAAATAGTGTTACGCAGAAAAAGTTCTTGTGTGACAGAACCATGTGGCGCATACCGTTCTC
AAGCAACTTCATGTCTATGGGAGCCCTTACAGACTTGGGACAGAACATGCTCTATGCCAACTCAGCTCATGCTCTGG
ACATGACCTTTGAGGTGGATCCCATGGATGAGCCCACCCTGCTTTATCTTCTCTTCGAAGTTTTCGACGTGGTCAGA
GTGCATCAGCCACACCGCGGCATCATCGAGGCAGTCTACCTGCGTACACCGTTCTCGGCCGGTAACGCTACCACGTA
AGAAGCTTCTTGCTTCTTGCAAACAGCAGCTGCAACCATGGCCTGCGGATCCCAAAACGGCTCCAGCGAGCAAGAGC
TCAGAGCCATTGTCCAAGACCTGGGTTGCGGACCATATTTTTTGGGAACCTTTGATAAGCGCTTCCCGGGGTTCATG
GCCCCCGATAAGCTCGCCTGTGCCATTGTAAATACGGCCGGACGTGAGACGGGGGGAGAGCACTGGTTGGCTTTCGG
TTGGAACCCACGTTCTAACACCTGCTACCTTTTTGATCCTTTTGGATTCTCGGATGATCGTCTCAAACAGATTTACC
AGTTTGAATATGAGGGTCTCCTGCGCCGCAGCGCTCTTGCTACCAAGGACCGGTGTATTACGCTGGAAAAATCTACC
CAGACCGTGCAGGGCCCCCGTTCTGCCGCCTGCGGACTTTTCTGCTGCATGTTCCTTCATGCCTTTGTGCACTGGCC
TGACCGTCCCATGGACGGAAACCCCACCATGAAATTGCTAACTGGAGTGCCAAACAACATGCTTCATTCTCCTAAAG
TCCAGCCCACCCTGTGTGACAATCAAAAAGCACTCTACCATTTTCTCAATACCCATTCGCCTTATTTTCGCTCTCAT
CGTACACACATCGAAAGGGCCACTGCGTTCGACCGTATGGATGTGCAATAATGATTCATGTAAACAACGTGTTCAAT
AAACAGCACTTTATTTTTTACATGTATCGAGGCTCTGGATTACTTATTTATTTACAAGTCGAATGGGTTCTGACGAG
AATCAGAATGACCCGCAGGCAGTGATACGTTGCGGAACTGATACTTGGGTTGCCACTTGAATTCGGGAATCACCAAC
TTGGGAACCGGTATATCGGGCAGGATGTCACTCCACAGCTTTCTGGTCAGCTGCAAAGCTCCCAGCAGGTCAGGAGC
CGAAATCTTGAAATCACAATTAGGACCAGTGCTCTGAGCGCGAGAGTTGCGGTACACCGGATTGCAGCACTGAAACA
CCATCAGCGACGGATGTCTTACGCTTGCCAGCACGGTGGGATCTGCAATCATGCCCACATCCAGATCTTCAGCATTG
GCAATGCTGAACGGGGTCATCTTGCAGGTCTGCCTACCCATGGCGGGCACCCAATTAGGCTTGTGGTTACAATCGCA
GTGCAGGGGGATCAGTATCATCTTGGCCTGATCCTGTCTGATTCCTGGATACACGGCTCTCATGAAAGCATCATATT
GCTTGAAAGCCTGCTGGGCTTTACTACCCTCGGTATAAAACATCCCGCAGGACCTGCTCGAAAACTGGTTAGCTGCG
CAGCCGGCATCATTCACACAGCAGCGGGCGTCATTGTTGGCTATTTGCACCACACTTCTGCCCCAGCGGTTTTGGGT
GATTTTGGTTCGCTCGGGATTCTCCTTCAAGGCTCGTTGTCCGTTCTCGCTGGCCACATCCATCTCGATAATCTGCT
CCTTCTGAATCATAATATTGCCATGCAAGCACTTCAGCTTGCCCTCATAATCATTGCAGCCATGAGGCCACAACGCA
CAGCCTGTACATTCCCAATTATGGTGGGCGATCTGAGAAAAAGAATGTATCATTCCCTGCAGAAATCTTCCCATCAT
CGTGCTCAGTGTCTTGTGACTAGTGAAAGTTAACTGGATGCCTCGGTGCTCCTCGTTCACGTACTGGTGACAGATGC
GCTTGTATTGTTCGTGCTGCTCAGGCATTAGTTTAAAAGAGGTTCTAAGTTCGTTATCCAGCCTGTACTTCTCCATC
AGCAGACACATCACTTCCATGCCTTTCTCCCAAGCAGACACCAGGGGCAAGCTAATCGGATTCTTAACAGTGCAGGC
AGCAGCTCCTTTAGCCAGAGGGTCATCTTTGGCGATCTTCTCAATGCTTCTTTTGCCATCCTTCTCAACGATGCGCA
CGGGCGGGTAGCTGAAACCCACTGCTACAAGTTGCGCCTCTTCTCTTTCTTCTTCGCTGTCTTGACTGATGTCTTGC
ATGGGGACATGTTTGGTCTTCCTTGGCTTCTTTTTCGGGGGTATCGGAGGAGGAGGACTGTCGCTCCGTTCCGGAGA
CAGGGAGGATTGTGACGTTTCGCTCACCATTACCAACTGACTGTCGGTAGAAGAACCTGACCCCACACGGCGACAGG
TGTTTCTCTTCGGGGGCAGAGGTGGAGGCGATTGCGAAGGGCTGCGGTCCGACCTGGAAGGCGGATGACTGGCAGAA
CCCCTTCCGCGTTCGGGGGTGTGCTCCCTGTGGCGGTCGCTTAACTGATTTCCTTCGCGGCTGGCCATTGTGTTCTC
CTAGGCAGAGAAACAACAGACATGGAAACTCAGCCATTGCTGTCAACATCGCCACGAGTGCCATCACATCTCGTCCT
CAGCGACGAGGAAAAGGAGCAGAGCTTAAGCATTCCACCGCCCAGTCCTGCCACCACCTCTACCCTAGAAGATAAGG
AGGTCGACGCATCTCATGACATGCAGAATAAAAAAGCGAAAGAGTCTGAGCCAGACATCGAACAAGACCCGGGCTAT
GTGACACCGGTGGAACACGAGGAAGAGTTGAAACGCTTTCTAGAGAGAGAGGATGAAAACTGCCCAAAACAGCAAGC
GGATAACTATCACCAAGATGCTGGAAATAGGGATCAGAACACCGACTACCTCATAGGGCTTGACGGGGAAGACGCGC
TCCTTAAACATCTAGCAAGACAGTCACTCATAGTCAAGGATGCATTATTGGACAGAACTGAAGTGCCCATCAGTGTC
GAAGAGCTCAGCCGCGCCTACGAGCTTAACCTATTTTCACCTCGTACTCCCCCCAAACGTCAGCCAAACGGCACCTG
CGAGCCAAATCCTCGCTTAAACTTTTATCCAGCTTTTGCTGTGCCAGAAGTACTGGCTACCTATCACATCTTTTTTA
AAAATCAAAAAATTCCAGTCTCCTGCCGCGCTAATCGCACCCGCGCCGATGCCCTACTCAATCTGGGACCTGGTTCA
CGCTTACCTGATATAGCTTCCTTGGAAGAGGTTCCAAAGATCTTCGAGGGTCTGGGCAATAATGAGACTCGGGCCGC
AAATGCTCTGCAAAAGGGAGAAAATGGCATGGATGAGCATCACAGCGTTCTGGTGGAATTGGAAGGCGATAATGCCA
GACTCGCAGTACTCAAGCGAAGCGTCGAGGTCACACACTTTGCATACCCCGCTGTCAACCTGCCCCCTAAAGTCATG
ACGGCCGTCATGGACCAGTTACTCATTAAGCGCGCAAGTCCCCTTTCAGAAGACATGCATGACCCAGATGCCTGTGA
TGAGGGTAAACCAGTGGTCAGTGATGAGCAGCTAACCCGATGGCTGGGCACCGACTCTCCCCGGGATTTGGAAGAGC
GTCGCAAGCTTATGATGGCCGTGGTGCTGGTTACCGTAGAACTAGAGTGTCTTCGGCGTTTCTTTACCGATTCAGAA
ACCTTGCGCAAACTCGAAGAGAATCTGCACTACACTTTTAGACACGGCTTTGTGCGGCAGGCATGCAAGATATCTAA
CGTGGAACTCACCAACCTGGTTTCCTACATGGGTATTCTGCATGAGAATCGCCTAGGACAAAGCGTGCTGCACAGCA
CCCTTAAGGGGGAAGCCCGCCGTGATTACATCCGCGATTGTGTTTATCTCTACCTGTGCCACACGTGGCAAACCGGC
ATGGGTGTATGGCAGCAATGTTTAGAAGAACAGAACCTGAAAGAGCTAAACAAGCTCTTACAGAAATCTCTTAAGGT
TCTGTGGACAGGGTTCGACGAGCGCACCGTCGCTTCCGACCTGGCAGACCTCATCTTCCCAGAGCGTCTCAGGGTTA
CTTTGCGAAACGGACTGCCTGACTTTATGAGCCAGAGCATGCTTAACAATTTTCGCTCTTTCATCCTGGAACGCTCC
GGTATCCTGCCCGCCACCTGCTGCGCACTGCCCTCCGACTTTGTGCCTCTCACCTACCGCGAATGCCCCCCGCCGCT
ATGGAGTCACTGCTACCTGTTCCGTCTGGCCAACTACCTCTCCTACCACTCGGATGTGATCGAGGATGTGAGCGGAG
ACGGCTTGCTGGAGTGTCACTGCCGCTGCAATCTGTGCACGCCCCACCGGTCCCTAGCTTGCAACCCCCAGTTGATG
AGCGAAACCCAGATAATAGGCACCTTTGAATTGCAAGGCCCCAGCAGCCAAGGCGATGGGTCTTCTCCTGGGCAAAG
TTTAAAACTGACCCCGGGACTGTGGACCTCCGCCTACTTGCGCAAGTTTGCCCCGGAAGATTACCACCCCTATGAAA
TCAAGTTCTATGAGGACCAATCACAGCCTCCGAAAGCCGAACTTTCGGCCTGCGTCATCACCCAGGGGGCAATTCTG
GCCCAATTGCAAGCCATCCAAAAATCCCGCCAAGAATTTCTACTGAAAAAGGGTAAGGGGGTCTACCTTGACCCCCA
GACCGGCGAGGAACTCAACACAAGGTTCCCTCAGGATGTCCCAACGACGAGAAAGCAAGAAGTTGAAGGTGCAGCCG
CCGCCCCCAGAAGATATGGAGGAAGATTGGGACAGTCAGGCAGAGGAAGCGGAGGAGGAGGACAGTCTGGAGGACAG
TCTGGAGGAAGACAGTTTGGAGGAGGAAAACGAGGAGGCAGAGGAGGTGGAAGAAGTAACCGCCGACAAACAGTTAT
CCTCGGCTGCGGAGACAAGCAACAGCGCTACCATCTCCGCTCCGAGTCGAGGAACCCGGCGGCGTCCCAGCAGTAGA
TGGGACGAGACCGGACGCTTCCCGAACCCAACCAGCGCTTCCAAGACCGGTAAGAAGGATCGGCAGGGATACAAGTC
CTGGCGGGGGCATAAGAATGCCATCATCTCCTGCTTGCATGAGTGCGGGGGCAACATATCCTTCACGCGGCGCTACT
TGCTATTCCACCATGGGGTGAACTTTCCGCGCAATGTTTTGCATTACTACCGTCACCTCCACAGCCCCTACTATAGC
CAGCAAATCCCGGCAGTCTCGACAGATAAAGACAGCGGCGGCGACCTCCAACAGAAAACCAGCAGCGGCAGTTAGAA
AATACACAACAAGTGCAGCAACAGGAGGATTAAAGATTACAGCCAACGAGCCAGCGCAAACCCGAGAGTTAAGAAAT
CGGATCTTTCCAACCCTGTATGCCATCTTCCAGCAGAGTCGGGGCCAAGAGCAGGAACTGAAAATAAAAAACCGATC
TCTGCGTTCGCTCACCAGAAGTTGTTTGTATCACAAGAGCGAAGATCAACTTCAGCGCACTCTCGAGGACGCCGAGG
CTCTCTTCAACAAGTACTGCGCGCTGACTCTTAAAGAGTAGGCAGCGACCGCGCTTATTCAAAAAAGGCGGGAATTA
CATCATCCTCGACATGAGTAAAGAAATTCCCACGCCTTACATGTGGAGTTATCAGCCCCAAATGGGATTGGCGGCAG
GCGCCTCCCAGGACTACTCCACCCGCATGAATTGGCTCAGCGCCGGGCCTTCTATGATTTCTCGAGTTAATGATATA
CGCGCCTACCGAAACCAAATACTTTTGGAACAGTCAGCTCTTACCACCACGCCCCGCCAACACCTTAATCCCAGAAA
TTGGCCCGCCGCCCTAGTGTACCAGGAAAGTCCCGCTCCCACCACTGTATTACTTCCTCGAGACGCCCAGGCCGAAG
TCCAAATGACTAATGCAGGTGCGCAGTTAGCGGGCGGCTCCACCCTATGTCGTCACAGGCCTCGGCATAATATAAAA
CGCCTGATGATCAGAGGCCGAGGTATCCAGCTCAACGACGAGTCGGTGAGCTCTCCGCTTGGTCTACGACCAGACGG
AATCTTTCAGATTGCCGGCTGCGGGAGATCTTCCTTCACCCCTCGTCAGGCTGTTCTGACTTTGGAAAGTTCGTCTT
CGCAACCCCGCTCGGGCGGAATCGGGACCGTTCAATTTGTGGAGGAGTTTACTCCCTCTGTCTACTTCAACCCCTTC
TCCGGATCTCCTGGGCACTACCCGGACGAGTTCATACCGAACTTCGACGCGATTAGCGAGTCAGTGGACGGCTACGA
TTGATGTCTGGTGACGCGGCTGAGCTATCTCGGCTGCGACATCTAGACCACTGCCGCCGCTTTCGCTGCTTTGCCCG
GGAACTCATTGAGTTCATCTACTTCGAACTCCCCAAGGATCACCCTCAAGGTCCGGCCCACGGAGTGCGGATTACTA
TCGAAGGCAAAATACACTCTCGCCTGCAACGAATTTTCTCCCAGCGGCCCGTGCTGATCGAGCGAGACCAGGGAAAC
ACCACGGTTTCCATCTACTGCATTTGTAATCACCCCGGATTGCATGAAAGCCTTTGCTGTCTTATGTGTACTGAGTT
TAATAAAAACTGAATTAAGACTCTCCTACGGACTGCCGCTTCTTCAACCCGGATTTTACAACCAGAAGAACGAAACT
TTTCCTGTCGTCCAGGACTCTGTTAACTTCACCTTTCCTACTCACAAACTAGAAGCTCAACGACTACACCGCTTTTC
CAGAAGCATTTTCCCTACTAATACTACTTTCAAAACCGGAGGTGAGCTCCAAGGTCTTCCTACAGAAAACCCTTGGG
TGGAAGCGGGCCTTGTAGTGCTAGGAATTCTTGCGGGTGGGCTTGTGATTATTCTTTGCTACCTATACACACCTTGC
TTCACTTTCCTAGTGGTGTTGTGGTATTGGTTTAAAAAATGGGGCCCATACTAGTCTTGCTTGTTTTACTTTCGCTT
TTGGAACCGGGTTCTGCCAATTACGATCCATGTCTAGACTTCGACCCAGAAAACTGCACACTTACTTTTGCACCCGA
CACAAGCCGCATCTGTGGAGTTCTTATTAAGTGCGGATGGGAATGCAGGTCCGTTGAAATTACACACAATAACAAAA
CCTGGAACAATACCTTATCCACCACATGGGAGCCAGGAGTTCCCGAGTGGTACACTGTCTCTGTCCGAGGTCCTGAC
GGTTCCATCCGCATTAGTAACAACACTTTCATTTTTTCTGAAATGTGCGATCTGGCCATGTTCATGAGCAAACAGTA
TTCTCTATGGCCTCCTAGCAAGGACAACATCGTAACGTTCTCCATTGCTTATTGCTTGTGCGCTTGCCTTCTTACTG
CTTTACTGTGCGTATGCATACACCTGCTTGTAACCACTCGCATCAAAAACGCCAATAACAAAGAAAAAATGCCTTAA
CCTCTTTCTGTTTACAGACATGGCTTCTCTTACATCTCTCATATTTGTCAGCATTGTCACTGCCGCTCACGGACAAA
CAGTCGTCTCTATCCCTCTAGGACATAATTACACTCTCATAGGACCCCCAATCACTTCAGAGGTCATCTGGACCAAA
CTGGGAAGCGTTGATTACTTTGATATAATCTGCAACAAAACAAAACCAATAATAGTAACTTGCAACATACAAAATCT
TACATTGATTAATGTTAGCAAAGTTTACAGCGGTTACTATTATGGTTATGACAGATACAGTAGTCAATATAGAAATT
ACTTGGTTCGTGTTACCCAGTTAAAAACCACGAAAATGCCAAATATGGCAAAGATTCGATCCGATGACAATTCTCTA
GAAACTTTTACATCTCCCACCACACCCGACGAAAAAAACATCCCAGATTCAATGATTGCAATTGTTGCAGCGGTGGC
AGTGGTGATGGCACTAATAATAATATGCATGCTTTTATATGCTTGTCGCTACAAAAAGTTTCATCCTAAAAAACAAG
ATCTCCTACTAAGGCTTAACATTTAATTTCTTTTTATACAGCCATGGTTTCCACTACCACATTCCTTATGCTTACTA
GTCTTGCAACTCTGACTTCTGCTCGCTCACACCTCACTGTAACTATAGGCTCAAACTGCACACTAAAAGGACCTCAA
GGTGGTCATGTCTTTTGGTGGAGAATATATGACAATGGATGGTTTACAAAACCATGTGACCAACCTGGTAGATTTTT
CTGCAACGGCAGAGACCTAACCATTATCAACGTGACAGCAAATGACAAAGGCTTCTATTATGGAACCGACTATAAAA
GTAGTTTAGATTATAACATTATTGTACTGCCATCTACCACTCCAGCACCCCGCACAACTACTTTCTCTAGCAGCAGT
GTCGCTAACAATACAATTTCCAATCCAACCTTTGCCGCGCTTTTAAAACGCACTGTGAATAATTCTACAACTTCACA
TACAACAATTTCCACTTCAACAATCAGCATTATCGCTGCAGTGACAATTGGAATATCTATTCTTGTTTTTACCATAA
CCTACTACGCCTGCTGCTATAGAAAAGACAAACATAAAGGTGATCCATTACTTAGATTTGATATTTAATTTGTTCTT
TTTTTTTTTATTTACAGTATGGTGAACACCAATCATGGTACCTAGAAATTTCTTCTTCACCATACTCATTTGTGCAT
TTAATGTTTGCGCTACTTTCACAGCAGTAGCCACAGCAACCCCAGACTGTATAGGAGCATTTGCTTCCTATGCACTT
TTTGCTTTTGTTACTTGCATCTGCGTATGTAGCATAGTCTGCCTGGTTATTAATTTTTTCCAACTTCTAGACTGGAT
CCTTGTGCGAATTGCCTACCTGCGCCACCATCCCGAATACCGCAACCAAAATATCGCGGCACTTCTTAGACTCATCT
AAAACCATGCAGGCTATACTACCAATATTTTTGCTTCTATTGCTTCCCTACGCTGTCTCAACCCCAGCTGCCTATAG
TACTCCACCAGAACACCTTAGAAAATGCAAATTCCAACAACCGTGGTCATTTCTTGCTTGCTATCGAGAAAAATCAG
AAATTCCCCCAAATTTAATAATGATTGCTGGAATAATTAATATAATCTGTTGCACCATAATTTCATTTTTGATATAC
CCCCTATTTGATTTTGGCTGGAATGCTCCCAATGCACATGATCATCCACAAGACCCAGAGGAACACATTCCCCTACA
AAACATGCAACATCCAATAGCGCTAATAGATTACGAAAGTGAACCACAACCCCCACTACTCCCTGCTATTAGTTACT
TCAACCTAACCGGCGGAGATGACTGAAACACTCACCACCTCCAATTCCGCCGAGGATCTGCTCGATATGGACGGCCG
CGTCTCAGAACAGCGACTTGCCCAACTACGCATCCGCCAGCAGCAGGAACGCGCGGCCAAAGAGCTCAGAGATGTCA
TCCAAATTCACCAATGCAAAAAAGGCATATTCTGTTTGGTAAAACAAGCCAAGATATCCTACGAGATCACCGCTACT
GACCATCGCCTCTCTTACGAACTTGGCCCCCAACGACAAAAATTTACCTGCATGGTGGGAATCAACCCCATAGTTAT
CACCCAGCAAAGTGGAGATACTAAGGGTTGCATTCACTGCTCCTGCGATTCCATCGAGTGCACCTACACCCTGCTGA
AGACCCTATGCGGCCTAAGAGACCTGCTACCAATGAATTAAAAAATGATTAATAAAAAATCACTTACTTGAAATCAG
CAATAAGGTCTCTGTTGAAATTTTCTCCCAGCAGCACCTCACTTCCCTCTTCCCAACTCTGGTATTCTAAACCCCGT
TCAGCGGCATACTTTCTCCATACTTTAAAGGGGATGTCAAATTTTAGCTCCTCTCCTGTACCCACAATCTTCATGTC
TTTCTTCCCAGATGACCAAGAGAGTCCGGCTCAGTGACTCCTTCAACCCTGTCTACCCCTATGAAGATGAAAGCACC
TCCCAACACCCCTTTATAAACCCAGGGTTTATTTCCCCAAATGGCTTCACACAAAGCCCAGACGGAGTTCTTACTTT
AAAATGTTTAACCCCACTAACAACCACAGGCGGATCTCTACAGCTAAAAGTGGGAGGGGGACTTACAGTGGATGACA
CTGATGGTACCTTACAAGAAAACATACGTGCTACAGCACCCATTACTAAAAATAATCACTCTGTAGAACTATCCATT
GGAAATGGATTAGAAACTCAAAACAATAAACTATGTGCCAAATTGGGAAATGGGTTAAAATTTAACAACGGTGACAT
TTGTATAAAGGATAGTATTAACACCTTATGGACTGGAATAAACCCTCCACCTAACTGTCAAATTGTGGAAAACACTA
ATACAAATGATGGCAAACTTACTTTAGTATTAGTAAAAAACGGAGGGCTTGTTAATGGCTACGTGTCTCTAGTTGGT
GTATCAGACACTGTGAACCAAATGTTCACACAAAAGACAGCAAACATCCAATTAAGATTATATTTTGACTCTTCTGG
AAATCTATTAACTGATGAATCAGACTTAAAAATTCCACTTAAAAATAAATCTTCTACAGCGACCAGTGAAACTGTAG
CCAGCAGCAAAGCCTTTATGCCAAGTACTACAGCTTATCCCTTCAACACCACTACTAGGGATAGTGAAAACTACATT
CATGGAATATGTTACTACATGACTAGTTATGATAGAAGTCTATTTCCCTTGAACATTTCTATAATGCTAAACAGCCG
TATGATTTCTTCCAATGTTGCCTATGCCATACAATTTGAATGGAATCTAAATGCAAGTGAATCTCCAGAAAGCAACA
TAGCTACGCTGACCACATCCCCCTTTTTCTTTTCTTACATTACAGAAGACGACAACTAAAATAAAGTTTAAGTGTTT
TTATTTAAAATCACAAAATTCGAGTAGTTATTTTGCCTCCACCTTCCCATTTGACAGAATACACCAATCTCTCCCCA
CGCACAGCTTTAAACATTTGGATACCATTAGAGATAGACATTGTTTTAGATTCCACATTCCAAACAGTTTCAGAGCG
AGCCAATCTGGGGTCAGTGATAGATAAAAATCCATCGCGATAGTCTTTTAAAGCGCTTTCACAGTCCAACTGCTGCG
GATGCGAATCCGGAGTCTGGATCACGGTCATCTGGAAGAAGAACGATGGGAATCATAATCCGAAAACGGTATCGGAC
GATTGTGTCTCATCAAACCCACAAGCAGCCGCTGTCTGCGTCGCTCCGTGCAACTGCTGTTTATGGGATCAGGGTCC
ACAGTGTCCTGAAGCATGATTTTAATAGCCCTTAACATCAACTTTCTGGTGCGATGCGCGCAGCAACGCATTCTGAT
TTCACTCAAATCTTTGCAGTAGGTACAACACATTATTACAATATTGTTTAATAAACCATAATTAAAAGCGCTCCAGC
CAAAACTCATATCTGATATAATCGCCCCTGCATGACCATCATACCAAAGTTTAATATAAATTAAATGACGTTCCCTC
AAAAACACACTACCCACATACATGATCTCTTTTGGCATGTGCATATTAACAATCTGTCTGTACCATGGACAACGTTG
GTTAATCATGCAACCCAATATAACCTTCCGGAACCACACTGCCAACACCGCTCCCCCAGCCATGCATTGAAGTGAAC
CCTGCTGATTACAATGACAATGAAGAACCCAATTCTCTCGACCGTGAATCACTTGAGAATGAAAAATATCTATAGTG
GCACAACATAGACATAAATGCATGCATCTTCTCATAATTTTTAACTCCTCAGGATTTAGAAACATATCCCAGGGAAT
AGGAAGCTCTTGCAGAACAGTAAAGCTGGCAGAACAAGGAAGACCACGAACACAACTTACACTATGCATAGTCATAG
TATCACAATCTGGCAACAGCGGGTGGTCTTCAGTCATAGAAGCTCGGGTTTCATTTTCCTCACAACGTGGTAACTGG
GCTCTGGTGTAAGGGTGATGTCTGGCGCATGATGTCGAGCGTGCGCGCAACCTTGTCATAATGGAGTTGCTTCCTGA
CATTCTCGTATTTTGTATAGCAAAACGCGGCCCTGGCAGAACACACTCTTCTTCGCCTTCTATCCTGCCGCTTAGCG
TGTTCCGTGTGATAGTTCAAGTACAGCCACACTCTTAAGTTGGTCAAAAGAATGCTGGCTTCAGTTGTAATCAAAAC
TCCATCGCATCTAATTGTTCTGAGGAAATCATCCACGGTAGCATATGCAAATCCCAACCAAGCAATGCAACTGGATT
GCGTTTCAAGCAGGAGAGGAGAGGGAAGAGACGGAAGAACCATGTTAATTTTTATTCCAAACGATCTCGCAGTACTT
CAAATTGTAGATCGCGCAGATGGCATCTCTCGCCCCCACTGTGTTGGTGAAAAAGCACAGCTAAATCAAAAGAAATG
CGATTTTCAAGGTGCTCAACGGTGGCTTCCAACAAAGCCTCCACGCGCACATCCAAGAACAAAAGAATACCAAAAGA
AGGAGCATTTTCTAACTCCTCAATCATCATATTACATTCCTGCACCATTCCCAGATAATTTTCAGCTTTCCAGCCTT
GAATTATTCGTGTCAGTTCTTGTGGTAAATCCAATCCACACATTACAAACAGGTCCCGGAGGGCGCCCTCCACCACC
ATTCTTAAACACACCCTCATAATGACAAAATATCTTGCTCCTGTGTCACCTGTAGCGAATTGAGAATGGCAACATCA
ATTGACATGCCCTTGGCTCTAAGTTCTTCTTTAAGTTCTAGTTGTAAAAACTCTCTCATATTATCACCAAACTGCTT
AGCCAGAAGCCCCCCGGGAACAAGAGCAGGGGACGCTACAGTGCAGTACAAGCGCAGACCTCCCCAATTGGCTCCAG
CAAAAACAAGATTGGAATAAGCATATTGGGAACCGCCAGTAATATCATCGAAGTTGCTGGAAATATAATCAGGCAGA
GTTTCTTGTAAAAATTGAATAAAAGAAAAATTTGCCAAAAAAACATTCAAAACCTCTGGGATGCAAATGCAATAGGT
TACCGCGCTGCGCTCCAACATTGTTAGTTTTGAATTAGTCTGCAAAAATAAAAAAAAAAACAAGCGTCATATCATAG
TAGCCTGACGAACAGGTGGATAAATCAGTCTTTCCATCACAAGACAAGCCACAGGGTCTCCAGCTCGACCCTCGTAA
AACCTGTCATGGTGATTAAACAACAGCACCGAAAGTTCCTCGCGGTGACCAGCATGAATAATTCTTGATGAAGCATA
CAATCCAGACATGTTAGCATCAGTTAACGAGAAAAAACAGCCAACATAGCCTTTGGGTATAATTATGCTTAATCGTA
AGTATAGCAAAGCCACCCCTCGCGGATACAAAGTAAAAGGCACAGGAGAATAAAAAATATAATTATTTCTCTGCTGC
TGTTCAGGCAACGTCGCCCCCGGTCCCTCTAAATACACATACAAAGCCTCATCAGCCATGGCTTACCAGACAAAGTA
CAGCGGGCACGCACAAGCTCTAAAGTCACTCTCCAACCTCTCCACAATATATATACACAAGCCCTAAACTGACGTAA
TGGGAGTAAAGTGTAAAAAATCCCGCCAAACCCAACACACACCCCGAAACTGCGTCACCAGGGAAAAGTACAGTTTC
ACTTCCGCAATCCCAACAAGCGTCACTTCCTCTTTCTCACGGTACGTCACATCCCATTAACTTGCAACGTCATTTTC
CCACGGCCGCGCCGCCCCGTTTAGCCGTTAACCCCACAGCCAATCACCACACACCCCACAATTTTTAAAATCACCTC
ATTTACATATTGGCACCATTCCATCTATAAGGTATATTATTGATGATG

GenBank Accession No. AAW33501 (SEQ ID NO: 232)

MTKRVRLSDSFNPVYPYEDESTSQHPFINPGFISPNGFTQSPDGVLTLKCLTPLTTTGGSLQLKVGGGLTVDDTDGT
LQENIRATAPITKNNHSVELSIGNGLETQNNKLCAKLGNGLKFNNGDICIKDSINTLWTGINPPPNCQIVENTNIND
GKLTLVLVKNGGLVNGYVSLVGVSDTVNQMFTQKTANIQLRLYFDSSGNLLTDESDLKIPLKNKSSTATSETVASSK
AFMPSTTAYPFNTTTRDSENYIHGICYYMTSYDRSLFPLNISIMLNSRMISSNVAYAIQFEWNLNASESPESNIATL
TTSPFFFSYITEDDN

GenBank Accession No. ABC49791 (SEQ ID NO: 233)

MRRVVLGGAVVYPEGPPPSYESVMQQQQATAVMQSPLEAPFVPPRYLAPTEGRNSIRYSELAPQYDTTRLYLVDNKS
ADIASLNYQNDHSNFLTTVVQNNDFTPTEASTQTINFDERSRWGGQLKTIMHTNMPNVNEYMFSNKFKARVMVSRKP
PEGVRVDDNYDHKQDILKYEWFEFTLPEGNFSVTMTIDLMNNAIIDNYLKVGRQNGVLESDIGVKFDTRNFKLGWDP
ETKLIMPGVYTYEAFHPDIVLLPGCGVDFTESRLSNLLGIRKKHPFQEGFKILYEDLEGGNIPALLDVDAYENSKKD
QKAKIEAAAEAKANIVANDPVRVANASEIRGDSFAATSVPTKESLLDDVSQNIELKLTIKPVEKDGKNRSYNVLEDK
INTAYRSWYLSYNYGDPEKGVRSWTLLTTSDVTCGAEQVYWSLPDMMQDPVTFRSTRQVSNYPVVGAELMPVFSKSF
YNEQAVYSQQLRQSTSLTHVFNRFPFNQILIRPPAPTITTVSENVPALTDHGTLPLRSSIRGVQRVTVTDARRRTCP
YVYKALGIVAPRVLSSRTF

GenBank Accession No. AAW33485 (SEQ ID NO: 234)

MATPSMLPQWAYMHIAGQDASEYLSPGLVQFARATDTYFNLGNKFRNPTVAPTHDVTTDRSQRLMLRFVPVDREDNT
YSYKVRYTLAVGDNRVLDMASTFFDIRGVLDRGPSFKPYSGTAYNSLAPKGAPNASQWLDKGVTSTGLVDDGNTDDG
EEAKKATYTFGNAPVKAEAEITKDGLPVGLEVSTEGPKPIYADKLYQPEPQVGDETWTDLDGKTEEYGGRVLKPETK
MKPCYGSFAKPTNIKGGQAKVKPKEDDGINNIEYDIDMNFFDLRSQRSELKPKIVMYAENVDLECPDTHVVYKPGVS
DASSETNLGQQSMPNRPNYIGFRDNFIGLMYYNSTGNMGVLAGQASQLNAVVDLQDRNTELSYQLLLDSLGDRTRYF
SMWNQAVDSYDPDVRVIENHGVEDELPNYCFPLDGVGPRIDSYKEIKPNGDQSTWINVDPTGSSELAKGNPFAMEIN
LQANLWRSFLYSNVALYLPDSYKYTPSNVTLPENKNTYDYMNGRVVPPSLVDTYVNIGARWSLDAMDNVNPFNHHRN
AGLRYRSMLLGNGRYVPFHIQVPQKFFAVKNLLLLPGSYTYEWNFRKDVNMVLQSSLGNDLRVDGASISFTSINLYA
TFFPMAHNTASTLEAMLRNDTNDQSFNDYLSAANMLYPIPANATNIPISIPSRNWAAFRGWSFTRLKTKETPSLGSG
FDPYFVYSGSIPYLDGTFYLNHTFKKVSIMFDSSVSWPGNDRLLSPNEFEIKRTVDGEGYNVAQCNMTKDWFLVQML
ANYNIGYQGFYIPEGYKDRMYSFFRNFQPMSRQVVDEVNYKDFKAVAIPYQHNNSGFVGYMAPTMRQGQPYPANYPY
PLIGTTAVNSVTQKKFLCDRIMWRIPFSSNFMSMGALTDLGQNMLYANSAHALDMTFEVDPMDEPTLLYLLFEVFDV
VRVHQPHRGIIEAVYLRTPFSAGNATT

GenBank Accession No. DQ900900 (SEQ ID NO: 206)

CATCATCATAATATACCCCACAAAGTAAACAAAAGTTAATATGCAAATGAGCTTTTGAATTTTAACGGTTTTGGGGC
GGAGCCAACGCTGATTGGACGAGAAGCGGTGATGCAAATAACGTCACGACGCACGGCTAACGGCCGGCGCGGAGGCG
TGGCCTAGGCCGGAAGCAAGTCGCGGGGCTAATGACGTATAAAAAAGCGGACTTTAGACCCGGAAACGGCCGATTTT
CCCGCGGCCACGCCCGGATATGAGGTAATTCTGGGCGGATGCAAGTGAAATTAGGTCATTTTGGCGCCAAAACTGAA
TGAGGAAGTGAAAAGTGAAAAATACCTGTCCCGCCCAGGGCGGAATATTTACCGAGGGCCGAGAGACTTTGACCGAT
TACGTGGGGTTTCGATTGCGGTGTTTTTTTCGCGAATTTCCGCGTCCGTGTGAAAGTCCGGTGTTTATGTCACAGAT
CAGCTGATCCACAGGGTATTTAAACCAGTTGAGCCCGTCAAGAGGCCACTCTTGAGTGCCAGCGAGTAGAGATTTCT
CTGAGCTCCGCTCCCAAAGTGTGAGAAAAATGAGACACCTGCGCCTCCTGTCTTCAACTGTGCCTATTAACATGGCC
GCATTATTGCTGGAGGACTATGTGAGTACAGTATTGGAGGACGAACTACATCCATCTCCATTTGAGCTGGGACCTAC
ACTTCAGGACCTTTATGATTTGGAGGTAGATGCCCATGATGACGACCCAAACGAAGAGGCTGTGAATTTAATATTTC
CAGAATCTCTGATTCTTCAGGCTGACATAGCCAGCGAAGCTGTACCTACACCACTTCATACACCGACTTTGTCACCC
ATACCTGAATTGGAAGAGGAGGACGAGTTAGACCTCCGATGTTATGAGGAAGGTTTTCCTCCCAGCGATTCAGAGGA
CGAACAGGGTGAGCAGAGCATGGCTCTAATCTCAGAATATGCTTGTGTGGTTGTGGAAGAGCATTTTGTGTTGGACA
ATCCTGAGGTGCCCGGGCAAGGCTGTAGATCCTGCCAGTACCACCGGGATAAGACCGGAGACACAAACGCCTCCTGC
GCTCTGTGTTACATGAAAAAGAACTTCAGCTTTATTTACAGTAAGTGGAGTGAATGTGAGAGAGGCTGAGTGCTTAA
CACATAACTGGGTGATGCTTAAACAGCTGTGCTAAGTGTGGTTTATTTTTGTTTCTAGGTCCGGTGTCAGAGGATGA
GTCATCACCCTCAGAAGAAAACCACCCGTGTCCCCCTGAGCTGTCAGGCGAAACGCCCCTGCAAGTGCACAAACCCA
CCCCAGTCAGACCCAGTGGCGAGAGGCGAGCAGCTGTTGAAAAAATTGAGGACTTGTTACATGACATGGGTGGGGAT
GAACCTTTGGACCTGAGCTTGAAACGCCCCAGGAACTAGGCGCAGCTGTGCTTAGTCATGTGTAAATAAAGTTGTAC
AATAAAAGTATATGTGACGCATGCAAGGTGTGGTTTATGACTCATGGGCGGGGCTTAGTCCTATATAAGTGGCAACA
CCTGGGCACTGGGCACAGACCTTCAGGGAGTTCCTGATGGATGTGTGGACTATCCTTGCAGACTTTAGCAAGACACG
CCGGCTTGTAGAGGATAGTTCAGACGGGTGCTCCGGGTTCTGGAGACACTGGTTTGGAACTCCTCTATCTCGACTGG
TGTACACAGTTAAGAAGGATTATAACGAGGAATTTGAAAATCTTTTTGCTGATTGCTCTGGCCTGCTAGATTCTCTG
AATCTCGGCCACCAGTCCCTTTTCCAGGAAAGGGTACTCCACAGCCTTGATTTTTCCAGCCCAGGGCGCACTACAGC
CGGGGTTGCTTTTGTGGTTTTTCTGGTTGACAAATGGAGCCAGAACACCCAACTGAGCAGGGGCTACATTCTGGACT
TCGCAGCCATGCACCTGTGGAGGGCATGGGTGAGGCAGCGGGGACAGAGAATCTTGAACTACTGGCTTATACAGCCA
GCAGCTCCGGGTCTTCTTCGTCTACACAGACAAACATCCATGTTGGAGGAAGAAATGAGGCAGGCCATGGACGAGAA
CCCGAGGAGCGGCCTGGACCCTCCGTCGGAAGAGGAGCTGGATTGAATCAGGTATCCAGCTTGTACCCAGAGCTTAG
CAAGGTGCTGACATCCATGGCTAGGGGAGTGAAGAGGGAGAGGAGCGATGGGGGCAATACCGGGATGATGACCGAGC
TGACGGCCAGCCTGATGAATCGCAAGCGCCCAGAGCGCATTACCTGGCACGAGCTACAGATGGAGTGCAGGGATGAG
TTGGGCCTGATGCAGGATAAATATGGCCTGGAGCAGATAAAAACACATTGGTTGAACCCAGATGAGGATTGGGAGGA
GGCCATTAAGAAATATGCCAAGATAGCCCTGCGCCCAGATTGCAAGTACATAGTGACCAAGACCGTGAATATTAGAC
ATGCCTGCTACATTTCAGGGAACGGGGCAGAGGTGGTCATCGATACCCTGGACAAGGCCGCCTTCAGGTGTTGCATG
ATGGGAATGAGAGCAGGAGTGATGAATATGAATTCCATGATCTTCATGAACATGAAGTTCAATGGAGAGAAGTTTAA
TGGGGTGCTGTTCATGGCCAACAGCCACATGACCCTGCATGGCTGCAGTTTCTTTGGCTTCAACAATATGTGCGCCG
AGGTCTGGGGCGCTTCCAAGATCAGGGGATGTAAGTTTTATGGCTGCTGGATGGGCGTGGTCGGAAGACCTAAGAGC
GAGATGTCTGTGAAGCAGTGTGTGTTTGAGAAATGCTACCTGGGAGTCTCTACCGAGGGCAATGCTAGAGTGAGACA
CTGCTCTTCCCTGGATACGGGCTGCTTCTGCCTGGTGAAGGGTACGGCCTCTCTGAAGCATAATATGGTGAAGGGCT
GCACAGATGAGCGCATGTACAACATGCTAACATGCGACTCGGGGGTCTGTCATATCCTGAAGAACATCCATGTGACC
TCCCACCCCAGAAAGAAGTGGCCAGTGTTTGAGAATAACCTGCTGATCAAGTGCCATATGCACCTGGGTGCCAGAAG
GGGCACCTTCCAGCCGTACCAGTGCAACTTTAGCCAGACCAAGCTGCTGTTGGAAAACGATGCCTTCTCCAGGGTGA
ACCTGAACGGCATCTTTGACATGGATGTCTCGGTGTACAAGATCCTGAGATACGATGAGACCAAGTCCAGGGTGCGC
GCTTGCGAGTGCGGGGGCAGACACACCAGGATGCAGCCAGTGGCCCTGGATGTGACCGAGGAGCTGAGACCAGACCA
CCTGGTGATGGCCTGTACCGGGACCGAGTTCAGCTCCAGTGGGGAGGACACAGATTAGAGGTAGGTTTGAGTAGTGG
GCGTGGCTAATGTGAGTATAAAGGCGGGTGTCTTACGAGGGTCTTTTTGCTTTTCTGCAGACATCATGAACGGGACC
GGCGGGGCCTTCGAAGGGGGGCTTTTTAGCCCTTATTTGACAACCCGCCTGCCGGGATGGGCCGGAGTTCGTCAGAA
TGTGATGGGATCTACGGTGGATGGGCGTCCAGTGCTTCCAGCAAATTCCTCGACCATGACCTACGCGACCGTGGGGA
GCTCGTCGCTTGACAGCACCGCCGCAGCCGCGGCAGCCGCAGCCGCCATGACAGCGACGAGACTGGCCTCGAGCTAT
ATGCCCAGCAGCGGTAGCAGCCCCTCTGTGCCCAGTTCCATCATCGCCGAGGAGAAACTGCTGGCCCTGCTGGCCGA
GCTGGAAGCCCTGAGCCGCCAGCTGGCCGCCCTGACCCAGCAGGTGTCCGATCTCCGCGAGCAACAGCAGCAGCAAA
ATAAATGATTCAATAAACACAGATTCTGATTCAAACAGCAAAGCATCTTTATTATTTATTTTTTCGCGCGCGGTAGG
CCCTGGTCCACCTCTCCCGATCATTGAGAGTGCGGTGGATTTTTTCCAGGACCCGGTAGAGGTGGGATTGGATGTTG
AGGTACATGGGCATGAGCCCGTCCCGGGGGTGGAGGTAGCACCACTGCATGGCCTCGTGCTCTGGGGTCGTGTTGTA
GATAATCCAGTCATAGCAGGGGCGCTGGGCGTGGTGCTGGATGATGTCCTTGAGGAGGAGACTGATGGCCACGGGGA
GCCCCTTGGTGTAGGTGTTGGCAAAGCGGTTAAGCTGGGAGGGATGCATGCGGGGGGAGATGATGTGCAGTTTGGCC
TGGATCTTGAGGTTGGCGATGTTGCCACCCAGATCCCGCCGGGGGTTCATATTGTGCAGGACCACCAGAACGGTGTA
GCCCGTGCACTTGGGGAACTTATCATGCAACTTGGAAGGGAATGCGTGGAAGAATTTGGAGACGCCCTTGTGCCCGC
CCAGGTTTTCCATGCACTCATCCATGATGATGGCAATGGGCCCGTGGGCTGCGGCTTTGGCAAAAACGTTTCTGGGG
TCAGAGACATCATAATTATGCTCCTGGGTGAGATCATCATAAGACATTTTAATGAATTTGGGGCGAAGGGTGCCAGA
TTGGGGGACGATCGTTCCCTCGGGCCCCGGGGCGAAGTTCCCCTCGCAGATCTGCATCTCCCAGGCTTTCATCTCGG
AGGGGGGGATCATGTCCACCTGCGGGGCGATGAAAAAAACGGTTTCCGGGGCGGGGGTGATGAGCTGCGAGGAGAGC
AGGTTTCTTAACAGCTGGGACTTGCCGCACCCGGTCGGGCCGTAGATGACCCCGATGACGGGTTGCAGGTGGTAGTT
CAAGGAGATGCAGCTGCCGTCGTCCCGGAGGAGGGGGGCCACCTCGTTGAGCATGTCTCTCACTTGGAGGTTTTCCC
GGACGAGCTCGCCGAGGAGGCGGTCCCCGCCCAGCGAGAGCAGCTCTTGCAGGGAAGCAAAGTTTTTCAGGGGCTTG
AGCCCGTCGGCCATGGGCATCTTGGCAAGGGTCTGCGAGAGGAGCTCCAGGCGGTCCCATAGCTCGGTGACGTGCTC
TACGGCATCTCGATCCAGCAGACTTCCTCGTTTCGGGGGTTGGGACGACTGCGACTGTAGGGCACGAGACGATGGGC
GTCCAGCGCGGCCAGCGTCATGTCCTTCCAGGGTCTCAGGGTCCGAGTGAGGGTGGTCTCCGTCACGGTGAAGGGGT
GGGCCCCGGGCTGGGCGCTTGCAAGGGTGCGCTTGAGACTCATCCTGCTGGTGCTGAAACGGGCACGGTCTTCGCCC
TGCGCGTCGGCGAGATAGCAGTTGACCATGAGCTTGTAGTTAAGGGCCTCGGCGGCGTGGCCCTTGGCACGGAGCTT
GCCTTTGGAAGAGCGCCCGCAGGCGGGACAGAGGAGGGATTGCAGGGCGTAGAGCTTGGGTGCGAGAAAGACGGACT
CGGGAGCGAAGGCGTCCGCTCCGCAGTGGGCGCAGACGGTTTCGCACTCGACGAGCCAGGTGAGCTCGGGCTGCTCG
GGGTCAAAAACCAGTTTTCCCCCGTTCTTTTTGATGCGCTTCTTACCTCGCGTCTCCATGAGTCTGTGTCCGCGTTC
GGTGACAAACAGGCTGTCTGTGTCCCCGTAGACGGACTTGATTGGCCTGTCCTGCAGGGGCGTCCCGCGGTCCTCCT
CGTAGAGAAACTCGGACCACTCTGAGACAAAGGCGCGCGTCCACGCCAAGACAAAGGAGGCCACGTGCGAGGGGTAG
CGGTCGTTGTCCACCAGGGGGTCCACCTTTTCCACCGTGTGCAGACACATGTCCCCCTCCTCCGCATCCAAGAAGGT
GATTGGCTTGTAGGTGTAGGCCACGTGACCGGGGGTCCCCGACGGGGGGGTATAAAAGGGGGCGGGTCTGTGCTCGT
CCTCACTCTCTTCCGCGTCGCTGTCCACGAGCGCCAGCTGTTGGGGTAGGTATTCCCTCTCGAGAGCGGGCATGACC
TCGGCACTCAGGTTGTCAGTTTCTAGAAACGAGGAGGATTTGATGTTGGCCTGCCCTGCCGCAATGCTTTTTAGGAG
ACTTTCATCCATCTGGTCAGAAAAGACTATTTTTTTATTGTCAAGCTTGGTGGCAAAGGAGCCATAGAGGGCGTTGG
AGAGAAGCTTGGCGATGGATCTCATGGTCTGATTTTTGTCACGGTCGGCGCGCTCCTTGGCCGCGATGTTGAGCTGG
ACATACTCGCGCGCGACACACTTCCATTCTGGGAAGACGGTGGTGCGCTCGTCGGGCACGATCCTGACGCGCCAGCC
GCGATTATGCAGGGTGACCAGGTCCACGCTGGTGGCCACCTCGCCGCGCAGGGGCTCGTTGGTCCAGCAGAGGCGTC
CGCCCTTGCGCGAGCAGAACGGGGGCAGCACATCAAGCAGATGCTCGTCAGGGGGGTCCGCATCGATGGTGAAGATG
CCCGGACAGAGTTCCTTGTCAAAATAATCGATTTTTGAGGATGCATCATCCAAGGCCATCTGCCACTCGCGGGCGGC
CAGCGCTCGCTCGTAGGGGTTGAGGGGCGGACCCCAGGGCATGGGATGCGTGAGGGCGGAGGCGTACATGCCGCAGA
TGTCGTAGACATAGATGGGCTCCGAGAGGATGCCGATGTAGGTGGGATAACAGCGCCCCCCGCGGATGCTGGCGCGC
ACATAGTCATACAACTCGTGCGAGGGGGCCAAGAAAGCGGGGCCGAGATTGGTGCGCTGGGGCTGCTCGGCGCGGAA
GACGATCTGGCGAAAGATGGCATGCGAGTTGGAGGAGATGGTGGGCCGTTGGAAGATGTTAAAGTGGGCGTGGGGCA
AGCGGACCGAGTCGCGGATGAAGTGCGCGTAGGAGTCTTGCAGCTTGGCAACGAGCTCGGCGGTGACAAGGACGTCC
ATGGCGCAGTAGTCCAGCGTTTCACGGATGATGTCATAACCCGCCTCTTCTTTCTTCTCCCACAGCGCGCGGTTGAG
GGCGTACTCCTCGTCATCCTTCCAGTACTCCCGGAGCGGGAATCCTCGATCGTCCGCACGGTAAGAGCCCAGCATGT
AGAAATGGTTCACGGCCTTGTAGGGACAGCAGCCCTTCTCCACGGGGAGGGCGTAAGCTTGAGCGGCCTTGCGGAGC
GAGGTGTGCGTCAGGGCGAAGGTATCCCTAACCATGACTTTCAAGAACTGGTACTTGAAATCCGAGTCGTCGCAGCC
GCCGTGCTCCCAGAGCTCGAAATCGGTGCGCTTCTTCGAGAGGGGGTTAGGCAGAGCGAAAGTGACGTCATTGAAGA
GAATCTTGCCTGCCCGCGGCATGAAATTGCGGGTGATGCGGAAAGGGCCCGGAACGGAGGCTCGGTTGTTGATGACC
TGGGCGGCGAGGACGATCTCGTCGAAGCCGTTGATGTTGTGCCCGACGATGTAGAGTTCCATGAATCGCGGGCGGCC
TTTGATGTGCGGCAGCTTTTTGAGTTCCTCGTAGGTGAGGTCCTCGGGGCATTGCAGGCCGTGCTGCTCGAGCGCCC
ACTCCTGGAGATGTGGGTTGGCTTGCATGAATGAAGCCCAGAGCTCGCGGGCCATGAGGGTCTGGAGCTCGTCGCGA
AAGAGGCGGAACTGCTGGCCCACGGCCATCTTTTCTGGGGTGACGCAGTAGAAGGTGAGGGGGTCCCGCTCCCAGCG
ATCCCAGCGTAAGCGCACGGCGAGATCGCGAGCGAGGGCGACCAGCTCGGGGTCCCCGGAGAATTTCATGACCAGCA
TGAAGGGGACGAGCTGCTTGCCGAAGGACCCCATCCAGGTGTAGGTTTCTACATCGTAGGTGACAAAGAGCCGCTCC
GTGCGAGGATGAGAGCCGATTGGGAAGAACTGGATTTCCTGCCACCAGTTGGTCGAGTGGCTGTTGATGTGATGAAA
GTAGAAATCCCGCCGGCGAACCGAGCACTCGTGCTGATGCTTGTAAAAGCGTCCGCAGTACTCGCAGCGCTGCACGG
GCTGTACCTCATCCACGAGATACACAGCGCGTCCCTTGAGGAGGAACTTCAGGAGTGGCGGCCCTGGCTGGTGGTTT
TCATGTTCGCCTGCGTGGGACTCACCCTGGGGCTCCTCGAGGACGGAGAGGCTGACGAGCCCGCGCGGGAGCCAGGT
CCAGATCTCGGCGCGGCGGGGGCGGAGAGCGAAAACGAGGGCGCGCAGTTGGGAGCTGTCCATGGTGTCGCGGAGAT
CCAGGTCCGGGGGCAGGGTTCTGAGGTTGACCTCGTAGAGGCGGGTGAGGGCGTGCTTGAGATGCAGATGGTACTTG
ATCTCCACGGGTGAGTTGGTGGTCGTGTCCACGCATTGCATGAGCCCGTAGCTGCGCGGGGCCACGACCGTGCCGCG
GTGCGCTTTTAGAAGCGGTGTCGCGGACGCGCTCCCGGCGGCAGCGGCGGTTCCGGCCCCGCGGGCAGTGGCGGTAG
AGGCACGTCGGCGTGGCGCTCGGGCAGGTCCCGGTGCTGCGCCCTGAGAGCGCTGGCGTGCGCGACGACGCGGCGGT
TGACATCCTGGATCTGCCGCCTTTGCGTGAAGACCACGGGCCCCGTGACTTTGAACCTGAAAGACAGTTCAACAGAA
TCAATCTCGGCGTCATTGACGGCGGCCTGACGCAGGATCTCTTGCACGTCGCCCGAGTTGTCCTGGTAGGCGATCTC
GGACATGAACTGCTCGATTTCCTCCTCCTGGAGATCGCCGCGGCCCGCGCGCTCTACGGTGGCGGCAAGGTCATTCG
AGATGCGACCCATGAGCTGCGAGAAGGCGCCCAGGCCGCTCTCGTTCCAGACGCGGCTGTAAACCACGTCCCCGTCG
GCGTCGCGCGCGCGCATGACCACCTGCGCGAGGTTGAGCTCCACGTGCCGCGCGAAGACGGCATAGTTGCGCAGGCG
TTGGAAGAGGTAGTTGAGGGTGGTGGCGATGTGCTCGGTGACGAAGAAGTACATAATCCAGCGGCGCAGGGGCATTT
CGCTGATGTCGCCAATGGCCTCCAGCCTTTCCATGGCCTCGTAGAAATCCACGGCGAAGTTGAAAAACTGGGCGTTG
CGGGCCGAGACCGTGAGCTCGTCTTCCAGGAGCCTGATGAGTTCGGCGATGGTGGCGCGCACCTCGCGCTCGAAATC
CCCGGGGGCCTCCTCCTCTTCCTCTTCTTCCATGACGACCTCTTCTTCTATTTCTTCCTCTGGGGGCGGTGGTGGTG
GCGGGGCCCGACGACGACGGCGACGCACCGGGAGACGGTCGACGAAGCGCTCGATCATCTCCCCGCGGCGGCGACGC
ATGGTTTCGGTGACGGCGCGACCCCGTTCGCGAGGACGCAGCGTGAAGACGCCGCCGGTCATCTCCCGGTAATGGGG
TGGGTCCCCGTTGGGCAGCGATAGGGCGCTGACAATGCATCTTATCAATTGCGGTGTAGGGCACGTGAGCGCGTCGA
GATCGACCGGATCGGAGAATCTTTCGAGGAAAGCGTCTAGCCAATCGCAGTCGCAAGGTAAGCTCAAACACGTAGCA
GCCCTGTGGACGCTGTTAGAATTGCGGTTGCTGATGATGTAATTGAAGTAGGCGTTTTTGAGGCGGCGGATGGTGGC
GAGGAGGACCAGGTCCTTGGGTCCCGCTTGCTGGATGCGGAGCCGCTCGGCCATGCCCCAGGCCTGGCCCTGACACC
GGCTCAGGTTCTTGTAGTAGTCATGCATGAGCCTCTCGATGTCATCACTGGCGGAGGCGGAGTCTTCCATGCGGGTG
ACCCCGACGCCCCTGAACGGCTGCACGAGCGCCAGGTCGGCGACGACGCGCTCGGCGAGGATGGCCTGTTGCACGCG
GGTGAGGGTGTCCTGGAAGTCGTCCATGTCGACGAAGCGGTGGTAGGCCCCTGTGTTGATGGTGTAAGTGCAGTTGG
CCATAAGCGACCAGTTGACGGTCTGCAGGCCGGGTTGCACGACCTCGGAGTACCTGAGCCGCGAGAAGGCGCGCGAG
TCGAAGACATAGTCGTTGCAGGTGCGCACGAGGTACTGGTATCCGACTAGAAAGTGCGGCGGCGGCTGGCGGTAGAG
CGGCCAGCGCTGGGTGGCCGGCGCGCCCGGGGCCAGGTCCTCAAGCATGAGTCGGTGGTAGCCGTAGAGGTAGCGGG
ACATCCAGGTGATGCCGGCGGCGGTGGTGGAGGCGCGCGGGAACTCGCGGACGCGGTTCCAGATGTTGCGCAGGGGC
AGGAAATAGTCCATGGTCGGCACGGTCTGGCCGGTGAGACGCGCGCAGTCATTGATGCTCTAGAGGCAAAAACGAAA
GCGGTTGAGCGGGCTCTTCCTCCGTAGCCTGGCGGAACGCAAACGGGTTAGGCCGCGTGTGTACCCCGGTTCGAGTC
CCCTCGAATCAGGCTGGAGCCGCGACTAACGTGGTATTGGCACTCCCGTCTCGACCCAAGCCCGATAGCCGCCAGGA
TACGGCGGAGAGCCCTTTTTGTCGGCCGAGGGGAGTCGCTAGACTTGAAAGCGGCCGAAAACCCTGCCGGGTAGTGG
CTCGCGCCCGTAGTCTGGAGAAGCATCGCCAGGGTTGAGTCGCGGCAGAACCCGGTTCAAGGACGGCCGCGGCGAGC
GGGACTTGGTCACCCCGCCGATTTAAAGACCCACAGCCAGCCGACTTCTCCAGTTACGGGAGCGAGCCCCCTTTTTT
CTTTTTGCCAGATGCATCCCGTCCTGCGCCAAATGCGTCCCACCCCCCCGGCGACCACCGCGACCGCGGCCGTAGCA
GGCGCCGGCGCTAGCCAGCCACAGCCACAGACAGAGATGGACTTGGAAGAGGGCGAAGGGCTGGCGAGACTGGGGGC
GCCGTCCCCGGAGCGACATCCCCGCGTGCAGCTGCAGAAGGACGTGCGCCCGGCGTACGTGCCTGCGCAGAACCTGT
TCAGGGACCGCAGCGGGGAGGAGCCCGAGGAGATGCGCGACTGCCGGTTTCGGGCGGGCAGGGAGCTGCGCGAGGGC
CTGGACCGCCAGCGCGTGCTGCGCGACGAGGATTTCGAGCCGAACGAGCAGACGGGGATCAGCCCCGCGCGCGCGCA
CGTGGCGGCGGCCAACCTGGTGACGGCCTACGAGCAGACGGTGAAGCAGGAGCGCAACTTCCAAAAGAGTTTCAACA
ACCACGTGCGCACCCTGATCGCGCGCGAGGAGGTGGCCCTGGGCCTGATGCACCTGTGGGACCTGGCGGAGGCCATC
GTGCAGAACCCGGACAGCAAGCCTCTGACGGCACAGCTGTTCCTGGTGGTGCAGCACAGCAGGGACAACGAGGCGTT
CAGGGAGGCACTGCTGAACATCGCCGAGCCCGAGGGTCGCTGGCTGCTGGAGCTGATTAACATCTTGCAGAGCATCG
TAGTGCAGGAGCGCAGCCTGAGCCTGGCCGAGAAGGTGGCGGCGATCAACTACTCGGTGCTGAGCCTGGGCAAGTTT
TACGCGCGCAAGATTTACAAGACGCCGTATGTGCCCATAGACAAGGAGGTGAAGATAGACAGCTTTTACATGCGCAT
GGCGCTCAAGGTGCTGACGCTGAGCGACGACCTGGGCGTGTACCGCAACGACCGCATCCACAAGGCCGTGAGCACAA
GCCGGCGGCGCGAGCTGAGCGACCGCGAGCTGATGCTGAGTCTGCGCCGGGCGCTGGTAGGAGGCGCCACCGGCGGT
GAGGAGTCCTACTTTGACATGGGGGCGGACCTGCATTGGCAGCCGAGCCGACGCGCCTTGGAGGCCGCCTACGGTCC
AGAGGACTTGGATGAGGAAGAGGAAGAGGAGGAGGATGCACCCGTTGCGGGGTACTGACGCCTCCGTGATGTGTTTT
TAGATGCAGCAAGCCCCGGACCCCGCCATAAGGGCGGCGCTGCAAAGCCAGCCGTCCGGTCTAGCATCGGACGACTG
GGAGGCCGCGATGCAACGCATCATGGCCCTGACGACCCGCAACCCCGAGTCCTTTAGACAACAGCCGCAGGCCAACA
GACTCTCGGCCATTCTGGAGGCGGTGGTTCCTTCTCGGACCAACCCCACGCACGAGAAGGTGCTGGCGATCGTGAAC
GCGCTGGCGGAGAACAAGGCCATCCGTCCCGACGAGGCCGGGCTAGTGTACAACGCCCTGCTGGAGCGCGTGGGCCG
CTACAACAGCACAAACGTGCAGTCCAACCTGGACCGGCTGGTGACGGACGTGCGCGAGGCCGTGGCGCAGCGCGAGC
GGTTCAAGAACGAGGGCCTGGGTTCGCTGGTGGCGCTGAACGCCTTCCTGGCGACGCAGCCGGCGAACGTGCCGCGC
GGGCAGGATGATTATACCAACTTTATAAGCGCGCTGCGGCTGATGGTGACCGAGGTGCCCCAGAGCGAGGTGTACCA
GTCGGGCCCGGACTACTTTTTCCAGACGAGCAGACAGGGCCTGCAGACGGTGAACCTGAGTCAGGCTTTCAAGAACC
TGCGCGGGCTGTGGGGCGTGCAGGCGCCCGTGGGCGACCGGTCGACGGTGAGCAGCTTGCTGACGCCCAACTCGCGG
CTGCTGCTGCTGCTGATCGCGCCCTTCACCGACAGTGGCAGCGTGAACCGCAACTCGTACCTGGGTCACCTGCTGAC
GCTGTACCGCGAGGCCATAGGCCAGGCGCAGGTGGATGAGCAGACCTTCCAGGAGATCACTAGCGTGAGCCGCGCGC
TGGGTCAGAACGACACCGACAGTCTGAGGGCCACCCTGAACTTCTTGCTGACCAATAGACAGCAGAAGATCCCGGCG
CAGTACGCGCTGTCGGCCGAGGAGGAGCGCATCCTGAGATATGTGCAGCAGAGCGTAGGGCTGTTCCTGATGCAGGA
GGGGGCCACCCCCAGCGCCGCGCTGGACATGACCGCGCGCAACATGGAACCTAGCATGTACGCCGCCAACCGGCCGT
TTATTAATAAGCTGATGGACTACCTGCACCGCGCGGCGTCCATGAACTCGGACTACTTTACCAATGCCATCTTGAAC
CCGCACTGGCTCCCGCCGCCGGGGTTCTACACGGGCGAGTACGACATGCCTGACCCCAACGACGGGTTTTTGTGGGA
CGACGTGGACAGCGCGGTGTTCTCACCGACCTTGCAAAAGCGCCAGGAGGCGGTGCGCACGCCCGCGAGCGAGGGCG
CGGTGGGTCGGAGCCCCTTTCCTAGCTTAGGGAGTTTGCATAGCTTGCCGGGCTCGGTGAACAGCGGCAGGGTGAGC
CGGCCGCGCTTGCTGGGCGAGGACGAGTACCTGAACGACTCGCTGCTGCAGCCGCCGCGGGTCAAGAACGCCATGGT
CAATAACGGGATAGAGAGTCTGGTGGACAAACTGAACCGCTGGAAAACCTACGCTCAGGACCATAGGGAACCTGCGC
CCGCGCCGCGGCGACAGCGTCACGACCGGCAGCGGGGCCTGGTGTGGGACGACGAGGACTCGGCCGACGATAGCAGC
GTGTTGGACTTGGGCGGGAGCGGTGGGGCCAACCCGTTCGCGCATCTGCAGCCCAGACTGGGGCGACGGATGTTTTG
AATGCAAAATAAAACTCACCAAGGCCATAGCGTGCGTTCTCTTCCTTGTTAGAGATGAGGCGCGCGGTGGTGTCTTC
CTCTCCTCCTCCCTCGTACGAGAGCGTGATGGCGCAGGCGACCCTGGAGGTTCCGTTTGTGCCTCCGCGGTATATGG
CTCCTACGGAGGGCAGAAACAGCATTCGTTACTCGGAGCTGGCTCCGCTGTACGACACCACTCGCGTGTATTTGGTG
GACAACAAGTCGGCGGACATCGCTTCCCTGAACTACCAAAACGACCACAGCAACTTCCTGACCACGGTGGTGCAGAA
CAACGATTTCACCCCTGCCGAGGCCAGCACGCAGACGATAAATTTTGACGAGCGGTCGCGGTGGGGCGGTGATCTGA
AGACCATTCTGCACACCAACATGCCTAATGTGAACGAGTACATGTTCACCAGCAAGTTTAAGGCGCGGGTGATGGTG
GCTAGAAAAAAGGCGGAAGGGGCTGATGCAAATGATAGGAGCAAGGATATCTTAGAGTATCAGTGGTTTGAGTTTAC
CCTGCCCGAGGGCAACTTTTCCGAGACCATGACCATAGACCTAATGAACAACGCCATCTTGGAAAACTACTTGCAAG
TGGGGCGGCAAAATGGCGTGCTGGAGAGTGATATCGGAGTCAAGTTTGACAGCAGAAATTTCAAGCTGGGCTGGGAC
CCGGTGACCAAGCTGGTGATGCCAGGGGTCTACACCTACGAGGCCTTCCACCCGGACGTGGTGCTGCTGCCGGGCTG
CGGGGTGGATTTCACCGAGAGCCGCCTGAGCAACCTCCTGGGCATTCGCAAGAAGCAACCTTTTCAAGAGGGCTTCA
GAATCATGTATGAGGACCTAGTAGGGGGCAACATCCCCGCTCTCCTGAATGTCAAGGAGTATCTGAAGGATAAGGAA
GAAGCTGGCAAAGCAGATGCAAATACTATTAAGGCTCAGAATGATGCCGTCCCAAGAGGAGATAACTATGCATCAGC
GGCAGAAGCCAAAGCAGCAGGAAAAGAAATTGAGTTGAAGGCCATTTTGAAAGATGATTCAGACAGAAGCTACAATG
TGATCGAGGGAACCACAGACACCCTGTACCGCAGTTGGTACCTGTCCTATACCTACGGGGATCCCGAGAAGGGGGTG
CAGTCGTGGACGCTGCTCACCACCCCGGACGTCACCTGCGGCGCGGAGCAAGTCTACTGGTCGCTGCCGGACCTCAT
GCAAGACCCCGTCACCTTCCGCTCTACCCAGCAAGTCAGCAACTACCCCGTGGTCGGCGCCGAGCTCATGCCCTTCC
GCGCCAAGAGCTTTTACAACGACCTCGCCGTCTACTCCCAGCTCATCCGCAGCTACACCTCCCTCACCCACGTCTTC
AACCGCTTCCCCGACAACCAGATCCTTTGCCGCCCGCCCGCGCCCACCATCACCACCGTCAGTGAAAACGTGCCTGC
TCTCACAGATCACGGGACGCTACCGCTGCGCAGCAGTATCCGCGGAGTCCAGCGAGTGACCGTCACTGACGCCCGTC
GCCGCACCTGTCCCTACGTCTACAAGGCCCTGGGCATAGTCGCGCCGCGCGTGCTTTCCAGTCGCACCTTCTAAAAA
ATGTCTATTCTCATCTCGCCCAGCAATAACACCGGCTGGGGTCTTACTAGGCCCAGCACCATGTACGGAGGAGCCAA
GAAACGCTCCCAGCAGCACCCCGTCCGCGTCCGCGGTCACTTCCGCGCTCCCTGGGGCGCTTACAAGCGGGGGCGGA
CCTCTGCTCCTGCCGCCGTGCGCACCACCGTCGACGACGTCATCGACTCGGTGGTCGCCGATGCGCGCAACTACACC
CCCGCCCCCTCGACCGTGGACGCGGTCATCGACAGCGTGGTGGCAGACGCGCGTGACTATGCCAGACGCAAGAGCCG
GCGGCGACGGATCGCCAGGCGCCACCGGAGCACGCCCGCCATGCGCGCCGCCCGAGCTCTGCTGCGCCGCGCCAGAC
GCACGGGCCGCCGGGCCATGATGCGAGCCGCGCGCCGCGCTGCCACTGCACCCACCCCCGCAGGCAGGACTCGCAGA
CGAGCGGCCGCTGCCGCCGCCGCGGCCATCTCTAGCATGACCAGACCCAGGCGCGGAAACGTGTACTGGGTGCGCGA
CTCCGTCACGGGCGTGCGCGTGCCCGTGCGCACCCGTCCTCCTCGTCCCTGATCTAATGCTTGTGTCCTCCCCCGCA
AGCGACGATGTCAAAGCGCAAAATCAAGGAGGAGATGCTCCAGGTCGTCGCCCCGGAGATTTACGGACCACCCCAGG
CGGACCAGAAACCCCGCAAAATCAAGCGGGTTAAAAAAAAGGATGAGGTGGACGAGGGGGCAGTAGAGTTTGTGCGC
GAGTTCGCTCCGCGGCGGCGCGTAAATTGGAAGGGGCGCAGGGTGCAGCGTGTGTTGCGGCCCGGCACGGCGGTGGT
GTTCACGCCCGGCGAGCGGTCCTCGGTCAGGAGCAAGCGTAGCTATGACGAGGTGTACGGCGACGACGACATCCTGG
ACCAGGCGGCGGAGCGGGCGGGCGAGTTCGCCTACGGGAAGCGGTCGCGCGAAGAGGAGCTGATCTCGCTGCCGCTG
GACGAAAGCAACCCCACGCCGAGCCTAAAGCCCGTGACCCTGCAGCAGGTGCTGCCCCAGGCAGTGCTGCTGCCGAG
CCGCGGGGTCAAGCGCGAGGGCGAGAGCATGTACCCGACCATGCAGATCATGGTGCCCAAGCGCCGGCGCGTGGAGG
ACGTGCTGGACACCGTAAAAATGGATGTGGAGCCCGAGGTCAAGGTGCGCCCCATCAAGCAGGTGGCGCCGGGCCTG
GGCGTGCAAACCGTGGACATTCAGATCCCCACCGACATGGATGTCGACAAAAAACCCTCGACCAGCATCGAGGTGCA
AACCGACCCCTGGCTCCCAGCCTCCACCGCTACCGCGTCCACTTCTACCGCCGCCACGGCTACCGAGCCTCCCAGGA
GGCGAAGATGGGGCGCCGCCAGCCGGCTGATGCCCAACTACGTGTTGCATCCTTCCATCATCCCGACGCCAGGCTAC
CGCGGCACCCGGTACTACGCCAGCCGCAGGCGCCCAGCCAGCAAACGCCGCCGCCGCACCGCCACCCGCCGCCGTCT
GGCCCCCGCCCGCGTGCGCCGCGTGACCACGCGGCGGGGCCGCTCGCTCGTTCTGCCCACCGTGCGCTACCACCCCA
GCATCCTTTAATCCGTGTGCTGTGATACTGTTGCAGAGAGATGGCTCTCACTTGCCGCCTGCGCATCCCCGTCCCGA
ATTACCGAGGAAGATCCCGCCGCAGGAGAGGCATGGCAGGCAGTGGCCTGAACCGCCGCCGGCGGCGGGCCATGCGC
AGGCGCCTGAGTGGCGGCTTTCTGCCCGCGCTCATCCCCATAATCGCCGCGGCCATCGGCACGATCCCGGGCATAGC
TTCCGTTGCGCTGCAGGCGTCGCAGCGCCGTTGATGTGCGAATAAAGCCTCTTTAGACTCTGACACACCTGGTCCTG
TATATTTTTAGAATGGAAGACATCAATTTTGCGTCCCTGGCTCCGCGGCACGGCACGCGGCCGTTCATGGGCACCTG
GAACGAGATCGGCACCAGCCAGCTGAACGGGGGCGCCTTCAATTGGAGCAGTGTCTGGAGCGGGCTTAAAAATTTCG
GCTCGACGCTCCGGACCTATGGGAACAAGGCCTGGAATAGTAGCACTGGGCAGTTGTTAAGGGAAAAGCTCAAAGAC
CAGAACTTCCAGCAAAAGGTGGTGGACGGGCTGGCCTCGGGCATTAACGGGGTGGTGGACATCGCGAACCAGGCCGT
GCAGCGCGAGATAAACAGCCGCCTGGACCCGCGGCCGCCCACGGTGGTGGAGATGGAAGATGCAACTCTTCCGCCGC
CCAAGGGCGAGAAGCGACCGCGGCCCGACGCGGAGGAGACAATCCTGCAAGTGGACGAGCCGCCCTCGTACGAGGAG
GCCGTCAAGGCCGGCATGCCCACCACGCGCATCATCGCGCCGCTGGCCACGGGTGTAATGAAACCCGCTACCCTTGA
CCTGCCTCCACCACCCACGCCCGCTCCACCAAAAGCAGCTCCGGTTGTGCAGCCCCCTCCGGTGGCGACCGCCGTGC
GCCGCGTCCCCGCCCGCCGCCAGGCCCAGAACTGGCAGAGCACGCTGCACAGTATCGTGGGCCTGGGAGTGAAAAGT
CTGAAGCGCCGCCGATGCTATTGAGAGAGAGTAAAGAGGACACTAAAGGGAGAGCTTAACTTGTATGTGCCTTACCG
CCAGAGAACGCGCGAAGATGGCCACCCCCTCGATGATGCCGCAGTGGGCGTACATGCACATCGCCGGGCAGGACGCC
TCGGAGTACCTGAGCCCGGGTCTGGTGCAGTTTGCCCGCGCCACCGACACGTACTTCAGCCTGGGCAACAAGTTTAG
GAACCCCACGGTGGCTCCCACCCACGATGTGACCACGGACCGGTCCCAGCGTCTGACGCTGCGCTTTGTGCCCGTGG
ATCGCGAGGACACCACGTACTCGTACAAGGCGCGCTTCACTCTGGCCGTGGGCGACAACCGGGTGCTAGACATGGCC
AGCACTTACTTTGACATCCGCGGCGTCCTGGACCGCGGCCCAAGCTTCAAACCCTACTCGGGCACGGCTTACAACAG
CCTGGCCCCCAAGGGCGCCCCCAATCCCAGTCAGTGGACTACCAAAGAAAAGCAAAACGGAGGAACTGGAGCAGAAA
AAGATGTTACAAAGACATTTGGACTTGCCGCCATGGGAGGCAGTAATATTTCTAAAGACGGTTTGCAGATTGGAACT
GACAAAACAGCAAATGCTGAAAAACCAATCTATGCAGACAAAACTTTCCAGCCAGAACCTCAAGTTGGAGAAGAAAA
CTGGCAGGATAATGATGAATATTATGGCGGCAGGGCTCTTAAAAAAGATACCAAAATGAAGCCATGCTATGGTTCAT
TTGCTAAACCCACAAACAAGGAAGGTGGGCAGGCTAAATTGAAAGAAACACCCAATGGTACCGATCCTCAATACGAT
GTGGACATGGCTTTCTTTGACTCAAGCACTATAAATATACCAGATGTGGTGTTGTACACTGAAAATGTAGATTTGGA
AACTCCAGATACACATGTGGTGTACAAACCAGGCAAAGAGGATGACAGTTCTGAAGCTAATTTAACTCAGCAGTCCA
TGCCTAACAGACCAAACTACATTGGCTTCAGAGACAACTTTGTGGGGCTATTGTACTACAACAGCACTGGCAACATG
GGTGTGCTGGCTGGTCAGGCTTCTCAGTTGAATGCCGTGGTCGACTTGCAAGACAGAAACACCGAACTGTCTTACCA
GCTCTTGCTAGATTCTCTTGGTGACAGAACCAGATATTTTAGTATGTGGAACTCTGCGGTGGACAGCTATGATCCCG
ATGTCAGGATCATTGAGAACCACGGTGTGGAAGATGAACTTCCTAACTATTGCTTCCCCTTGGACGGTGTTCAAACT
AATTCAGCCTATCAAGGTGTTAAACTAAAGCCTGATCAAACAGGAGGCGGAGTTAATGGAGATTGGGTAAAGGATGA
TGACATTTCAGCCCATAATCAAATTGGAAAGGGCAACATCTTTGCCATGGAGATCAACCTCCAGGCCAACCTGTGGA
AGAGTTTTCTGTACTCGAACGTGGCCCTGTACCTGCCCGACTCCTACAAGTACACGCCGGCCAACGTCACGCTGCCC
GCCAACACCAACACCTATGAGTACATGAACGGCCGCGTGGTAGCCCCCTCGCTGGTGGACGCCTACATTAACATCGG
CGCCCGCTGGTCGCTGGACCCCATGGACAACGTCAACCCCTTTAACCACCACCGCAATGCGGGCCTGCGCTACCGCT
CCATGCTTTTGGGCAATGGCCGCTACGTGCCCTTCCACATCCAAGTGCCCCAAAAGTTCTTTGCCATCAAGAACCTG
CTCCTGCTCCCCGGCTCCTACACCTACGAGTGGAACTTCCGCAAGGATGTCAACATGATCCTGCAGAGTTCCCTCGG
AAACGACCTGCGCGTCGACGGCGCCTCCGTCCGCTTCGACAGCGTCAACCTCTACGCCACCTTCTTCCCCATGGCGC
ACAACACCGCCTCCACCCTGGAAGCCATGCTGCGCAACGACACCAACGACCAGTCCTTCAACGACTACCTCTCGGCC
GCCAACATGCTCTACCCCATCCCGGCCAAGGCCACCAACGTGCCCATTTCCATCCCCTCGCGCAACTGGGCCGCCTT
CCGCGGCTGGAGTTTCACCCGGCTCAAGACCAAGGAAACTCCCTCCCTTGGCTCGGGTTTTGACCCCTACTTTGTCT
ACTCGGGCTCCATCCCCTACCTCGACGGGACCTTCTACCTCAACCACACCTTCAAGAAGGTTTCCATCATGTTCGAC
TCCTCGGTCAGCTGGCCCGGCAACGACCGGCTGCTTACGCCGAACGAGTTCGAGATCAAGCGCAGCGTCGACGGGGA
GGGCTACAACGTGGCCCAATGCAACATGACCAAGGACTGGTTCCTCGTCCAGATGCTCTCCCACTACAACATCGGCT
ACCAGGGCTTCCATGTGCCCGAGGGCTACAAGGACCGCATGTACTCCTTCTTCCGCAACTTCCAGCCCATGAGCAGG
CAGGTGGTCGATGAGATCAACTACAAGGACTACAAGGCAGTCACCCTGCCCTTCCAGCACAACAACTCTGGCTTCAC
CGGCTACCTGGCACCCACCATGCGTCAGGGGCAGCCCTACCCCGCCAACTTCCCCTACCCGCTCATCGGCTCCACCG
CAGTGCCATCCGTCACCCAGAAAAAGTTCCTCTGCGACAGGGTCATGTGGCGCATCCCCTTCTCCAGCAACTTCATG
TCCATGGGCGCCCTCACCGACCTGGGTCAGAACATGCTCTACGCCAACTCGGCCCACGCGCTCGACATGACCTTCGA
GGTGGACCCCATGGATGAGCCCACCCTCCTCTATCTTCTCTTCGAAGTTTTCGACGTGGTCAGAGTGCACCAGCCGC
ACCGCGGCGTCATCGAGGCCGTCTACCTGCGCACGCCCTTCTCCGCCGGCAACGCCACCACCTAAGCATGAGCGGCT
CCAGCGAACGAGAGCTCGCGGCCATCGTGCGCGACCTGGGCTGCGGGCCCTACTTTTTGGGCACCCACGACAAGCGC
TTCCCGGGCTTCCTCGCCGGCGACAAGCTGGCCTGCGCCATCGTCAACACGGCCGGCCGCGAGACCGGGGGCGTGCA
CTGGCTCGCCTTTGGCTGGAACCCGCGCTCGCGCACCTGCTACATGTTCGACCCCTTCGGGTTCTCGGACCGCCGGC
TCAAGCAGATTTACAGCTTCGAGTACGAGGCCATGCTGCGCCGAAGCGCCCTGGCCTCCTCGCCCGATCGCTGTCTT
AGCCTCGAACAGTCCACCCAGACCGTGCAGGGGCCCGACTCCGCCGCCTGCGGACTCTTCTGTTGCATGTTCTTGCA
TGCCTTCGTGCACTGGCCCGACCGACCCATGGACGGGAACCCCACCATGAACTTGCTGACGGGGGTGCCCAACGGCA
TGCTACAATCGCCACAGGTGCTGCCCACCCTCAGGCGCAACCAGGAGGAGCTCTACCGCTTCCTCGCGCGCCACTCC
CCCTACTTTCGCTCCCACCGCGCCGCCATCGAACACGCCACCGCTTTTGATAAAATGAAACAACTGCGTGTATGACT
CAAATAAACAGCACTTTTATTTTACACATGCGCTGGAGTATATGCAAGTTATTTAAAAGTCGAAGGGGTTCTCGCGC
TCGTCGTTGTGCGCCGCGCTGGGGAGGGCCACGTTGCGGTACTGGAACTTGGGCTGCCACTTGAACTCGGGGATCAC
CAGTTTGGGCACTGGAGTCTCGGGGAAGGTCTCGCTCCACATGCGCCGGCTCATTTGCAGGGCGCCCAGCATGTCAG
GGCCGGAGATCTTGAAATCGCAGTTGGGACCGGTGCTCTGCGCGCGCGAGTTGCGGTACACGGGGTTGCAGCACTGG
AACACCATCAGACTGGGGTACTTCACACTGGCAAGCACGCTCTTGTCGCTAATCTGATCCTTGTCCAGGTCCTCGGC
GTTGCTCAGGCCGAACGGGGTCATCTTGCACAGCTGGCGGCCCAGGAAGGGCACGCTCTGAGGCTTGTGGTTACACT
CGCAGTGCACGGGCATCAGCATCATCCCCGCGCCGCGCTGCATATTCGGGTAGAGGGCCTTGACGAAGGCCGCGATC
TGCTTGAAAGCTTGCTGGGCCTTGGCCCCCTCGCTAAAAAACAGGCCGCAGCTCTTCCCGCTGAACTGGTTATTCCC
GCACCCGGCATCATGCACGCAGCAGCGCGCGTCATGGCTGGTCAGTTGCACCACGCTCCGTCCCCAGCGGTTCTGGG
TCACCTTAGCCTTGCTGGGCTGCTCCTTCAGCGCGCGCTGTCCGTTCTCGCTGGTCACATCCATCTCCACCACGTGG
TCCTTGTGAATCATCACCGTTCCATGCAGACACTTGAGCTGACCTTCCACCTCGGTGCAGCCGTGATCCCACAGGAC
GCAGCCGGTGCACTCCCAATTCTTGTGCGCGATCCCGCTGTGGCTGAAAATGTAACCTTGCAACAGGCGACCCATAA
TGGTGCTAAATGCTTTCTGGGTGGTGAATGTCAGTTGCATCCCGCGGGCCTCCTCGTTCATCCAGGTCTGGCACATC
TTCTGGAAGATCTCGGTCTGCTCCGGCATGAGCTTGTAAGCATCGCGCAAGCCGCTGTCGACGCGGTAGCGTTCCAT
CAGCACGTTCATGGTATCCATGCCCTTCTCCCATGACGAGACCAGAGGCAGACTCAGGGGGTTGCGCACGTTCAGGA
CACCAGGGGTCGCGGGCTCGACGATGCGTTTTCCGTCCTTGCCTTCCTTCAACAGAACCGGAGGCTGGCTGAATCCC
ACTCCCACGATCACGGCGTCTTCCTGGGGCATCTCTTCGTCGGGGTCTACCTTGGTCACATGCTTGGTCTTTCTGGC
TTGCTTCTTTTTTGGAGGGCTGTCCACGGGGACCACGTCCTCCTCGGAAGACCCGGAGCCCACCCGCTGATACTTTC
GGCGCTTGGTGGGCAGAGGAGGTGGCGGCGGCGAGGGGCTCCTCTCCTGCTCCGGCGGATAGCGCGCCGACCCGTGG
CCCCGGGGCGGAGTGGCCTCTCGCTCCATGAACCGGCGCACGTCCTGACTGCCGCCGGCCATTGTTTCCTAGGGGAA
GATGGAGGAGCAGCCGCGTAAGCAGGAGCAGGAGGAGGACTTAACCACCCACGAGCAACCCAAAATCGAGCAGGACC
TGGGCTTCGAAGAGCCGGCTCGTCTAAAACCCCCACAGGATGAACAGGAGCACGAGCAAGACGCAGGCCAGGAGGAG
ACCGACGCTGGGCTCGAGCATGGCTACCTGGGAGGAGAGGAGGATGTGCTGCTAAAACACCTGCAGCGCCAGTCCCT
CATCCTCCGGGACGCCCTGGCCGACCGGAGCGAAACCCCCCTCAGCGTCGAGGAGCTGTGTCGGGCCTACGAGCTCA
ACCTCTTCTCGCCGCGCGTGCCCCCCAAACGCCAGCCCAACGGCACCTGCGAGCCCAACCCGCGTCTCAACTTCTAT
CCCGTCTTTGCGGTCCCCGAGGCCCTTGCCACCTATCACATCTTTTTCAAGAACCAAAAGATCCCCATCTCCTGTCG
CGCCAATCGCACTCGCGCCGACGCGCTCCTCGCTCTGGGGCCCGGCGCGCGCATACCTGATATCGCTTCCCTGGAAG
AGGTGCCCAAGATCTTCGAAGGGCTCGGTCGGGACGAGACGCGCGCGGCAAACGCTCTGAAAGAAACAGCAGAGGAA
GAGGGTTACACTAGCGCCCTGGTAGAGTTGGAAGGCGACAACGCCAGGCTGGCCGTGCTTAAGCGCAGCGTCGAGCT
CACCCATTTCGCCTACCCCGCCGTCAACCTCCCGCCCAAGGTCATGCGTCGCATCATGGATCAGCTCATCATGCCCC
ACATCGAGGCCCTTGATGAAAGTCAGGAACAGCGCCCCGAGAACGCCCAGCCCGTGGTCAGCGACGAGATGCTCGCG
CGCTGGCTCGGGACCCGCGACCCCCAGGCCCTGGAGCAGCGGCGCAAGCTCATGCTGGCCGTGGTCCTGGTCACCCT
TGAGCTCGAATGCATGCGCCGCTTTTTTACCGACCCCGAGACCCTGCGCAAGGTCGAGGAGACCCTGCACTACACTT
TCAGACACGGTTTCGTCAGGCAGGCCTGCAAGATCTCCAACGTGGAGCTGACCAACCTGGTCTCCTGCCTGGGGATC
CTACACGAGAACCGCTTGGGACAGACCGTGCTCCACTCTACCCTGAAGGGCGAGGCGCGGCGGGACTACATCCGCGA
CTGCGTCTTTCTCTTTCTCTGCCACACATGGCAAGCGGCCATGGGCGTGTGGCAGCAGTGTCTCGAGGACGAGAACC
TGAAGGAGCTGGACAAGCTTCTTGCTAGAAACCTTAAAAAGCTGTGGACGGGCTTCGACGAGCGCACCGTCGCCTCG
GACCTGGCCGAGATCGTCTTCCCCGAGCGCCTGAGGCAGACGCTGAAAGGAGGGCTGCCCGACTTCATGAGCCAGAG
CATGTTGCAAAACTACCGCACTTTCATTCTCGAGCGATCTGGGATGCTGCCCGCCACCTGCAACGCCTTCCCCTCCG
ACTTTGTCCCGCTGAGCTACCGCGAGTGTCCCCCGCCGCTGTGGAGCCACTGCTACCTCTTGCAGCTGGCCAACTAC
ATTGCCCACCACTCGGATGTGATCGAGGACGTGAGCGGCGAGGGGCTGCTCGAGTGCCACTGTCGCTGCAACCTATG
CTCCCCGCACCGCTCCCTGGTCTGCAACCCCCAGCTACTGAGCGAGACCCAGGTCATCGGTACCTTTGAGCTGCAAG
GTCCGCAGGAGTCCACCGCTCCGCTGAAACTCACGCCGGGGTTGTGGACTTCCGCGTACCTGCGCAAATTTGTACCC
GAGGACTACTACGCCCATGAGATAAAGTTCTTCGAGGACCAATCGCGTCCGCAGCACGCGGATCTCACGGCCTGCGT
CATCACCCAGGGCGCGATCCTCGCCCAATTGCACGCCATCCAAAAATCCCGCCAAGAGTTTCTTCTGAAAAAGGGTA
GAGGGGTCTACCTGGACCCCCAGACGGGCGAGGTGCTCAACCCGGGTCTCCCCCAGCATGCCGAGGAAGAAGCAGGA
GCCGCTAGTGGAGGAGATGGAAGAAGAATGGGACAGCCAGGCAGAGGAGGACGAATGGGAGGAGGAGACAGAGGAGG
AAGACTTGGAAGAGGTGGAAGAGGAGCAGGCAACAGAGCAGCCCGTCGCCGCACCATCCGCGCCGGCAGCCCCTCCG
GTCACGGATACAACCTCCGCAGCTCCGGCCAAGCCTCCTCGTAGATGGGATCGAGTGAAGGGTGACGGTAAGCACGA
GCGACAGGGCTACCGATCATGGAGGGCCCACAAAGCCGCGATCATCGCCTGCTTGCAAGACTGCGGGGGGAACATCG
CTTTCGCCCGCCGCTACCTGCTCTTCCACCGCGGGGTGAACATCCCCCGCAACGTGTTGCATTACTACCGTCACCTT
CACAGCTAAGAAAAAGCAAGTCAAAGGAGTCGCCGGAGGAGGAGGCCTGAGGATCGCGGCGAACGAGCCCTTGACCA
CCAGGGAGCTGAGGAACCGGATCTTCCCCACTCTTTATGCCATTTTTCAGCAAAGTCGAGGTCAGCAGCAAGAGCTC
AAAGTAAAAAACCGGTCTCTGCGCTCGCTCACCCGCAGTTGCTTGTACCACAAAAACGAAGATCAGCTGCAGCGCAC
TCTCGAAGACGCCGAGGCTCTGTTCCACAAGTACTGCGCGCTGACTCTTAAAGACTAAGGCGCGCCCACCCGGAAAA
AAGGCGGGAATTACCTCATCGCCACCATGAGCAAGGAGATTCCCACCCCTTACATGTGGAGCTATCAGCCCCAGATG
GGCCTGGCCGCGGGCGCCTCCCAGGACTACTCCACCCGCATGAACTGGCTTAGTGCCGGCCCCTCGATGATCTCACG
GGTCAACGGGGTCCGTAACCATCGAAACCAGATATTGTTGCAGCAGGCGGCGGTCACCTCCACGCCCAGGGCAAAGC
TCAACCCGCGTAATTGGCCCTCCACCCTGGTGTATCAGGAAATCCCCGGGCCGACTACCGTACTACTTCCGCGTGAC
GCACTGGCCGAAGTCCGCATGACTAACTCAGGTGTCCAGCTGGCCGGCGGCGCTTCCCGGTGCCCGCTCCGCCCACA
ATCGGGTATAAAAACCCTGGTGATCCGAGGCAGAGGCACACAGCTCAACGACGAGTTGGTGAGCTCTTCAATCGGTC
TGCGACCGGACGGAGTGTTCCAACTAGCCGGAGCCGGGAGATCGTCCTTCACTCCCAACCAGGCCTACCTGACCTTG
CAGAGCAGCTCTTCGGAGCCTCGCTCGGGAGGCATCGGAACCCTCCAGTTCGTGGAGGAGTTTGTGCCCTCGGTCTA
CTTCAACCCCTTCTCGGGCTCGCCAGGCCTCTACCCGGACGAGTTTATACCGAACTTCGACGCAGTGAGAGAAGCGG
TGGACGGCTACGACTGAATGTCCTATGGTGACTCGGCTGAGCTCGCTCGGTTGAGGCATCTGGACCACTGCCGCCGC
CTGCGCTGCTTTGCCCGGGAGAGCTACGGCCTCATCTACTTTGAGCTGCCCGAGGAGCACCCCAACGGCCCTGCACA
CGGAGTGCGGATCACCGTAGAGGGCACCACCGAGTCTCACCTGGTCAGGTTCTTCACCCAGCAACCCTTCCTGGTCG
AGCGGGACCGGGGCGCCACCACCTACACCGTCTACTGCATTTGTCCTACCCCGAAGTTGCATGAGAATTTTTGTTGT
ACTCTTTGTGGTGAGTTTAATAAAAGCTAAACTCTTGCAATACTCTGGACCTTGTCGTCATCAACTCAACGAGACCG
TCTACCTCACCAACCAGACTGAGGTAAAACTTACCTGCAGACCACACAAGACCTATATCATCTGGTTCTTCGAGAAC
ACCTCATTTGCAGTCTCCAACACTCACTGCAACGACGGTGTTGAACTTCCCAACAACCTTTCCAGTGGACTGAGTTA
CAATACACGTAGAGCTAAGCTCATCCTCTACAATCCTTTTGTAGAGGGAACCTACCAGTGCCAGAGCGGACCTTGCT
TCCACAGTTTTACTTTGGTGAACGTTACCGGCAGCAGCACAGCCGCTCCAGAAACTAACCTTCCTTCTGATACTATC
AAACCTTGTTTCGGAGGTGAGCTAAGGCTTCCCCCTTCTCAGGAGGGGGTTAGCCCATACGAAGTGGTCGGGTATTT
GATTTTAGGGGTGGTCCTGGGTGGGTGCATAGCGGTGCTAGCTCAGCTGCCTTGCTGGGTGGAAATCAAAATCTTTA
TATGCTGGGTAAGACATTGTGGGGAGGAACTATGAAGGGGCTCTTGCTGATTATCCTTTCCCTGGTGGGGGGTGTGC
TGTCATGCCACGAACAGCCACGATGTAACATCACCACAGGCAATGAGAGGAACGACTGCTCTGTAGTTATCAAATGC
GAGCACCATTGTCCTCTCAACATTACATTCAAAAATAAGACCATGGGAAATGTATGGGTGGGATTCTGGCAACCAGG
AGATGAGCAGAACTACACGGTCACTGTCCATGGTAGCAATGGCAATCACACTTTCGGTTTCAAATTCATTTTTGAAG
TCATGTGTGATATCACACTACATGTGGCTAGACTTCATGGCTTGTGGCCCCCTACCAAGGATAACATGGTGGGTTTT
TCTTTGGCTTTTGTGATCATGGCCTGCTTGATGTCAGGTCTGCTGGTAGGGGCTCTAGTGTGGTTTCTGAAACGCAA
GCCCAGGTATGGAAATGAAGAGAAGGAAAAATTGCTATAAATTCTTTTTCTTTTTCGCAGAACCATGAATACAGTGA
TCCGTATCGTGCTGCTCTCTCTTCTTGTAGCTTTTAGTCAGGCAGGATTTCATACTATCAATGCTACATGGTGGGCT
AATATAACTTTAGTGGGACCCCCAGACACACCAGTCACTTGGTATGATACTCAAGGATTGTGGTTTTGCAATGGCAG
TAGAGTTAAGAATCCTCAAATCAGACATACATGTAATGATCAAAACCTTACTTTGATCCATGTGAACAAAACTTATG
AAAGAACATACATGGGTTATAATAGACAAGGGACTAAAAAAGAAGACTACAAAGTTGTAGTTATACCACCTCCTCCT
GCTACTGTAAAACCACAGCCAGAGCCAGAGTATGTGTTTGTTTATATGGGAGAGAACAAAACTCTAGAAGGTCCTCC
GGGAACTCCAGTCACATGGTTTAATCAGGATGGAAAGAAATTTTGTGAAGGAGAAAAAGTTCTTCATCCAGAATTTA
ACCACACCTGTGACAAACAAAACCTTATACTACTGTTTGTGAATTTTACACATGATGGAGCTTACCTTGGGTACAAT
CATCAAGGAACCCAGAGAACACACTATGAAGTTACAGTATTAGATCTTTTTCCAGATTCTGGCCAAATGAAAATTGA
AAATCATAGTGAGGAAACAGAGCAAAAAAATGATGAACATCATAACTGGCAGAAACAGGGTGGGCAAAAACAGGGTG
GGCAAAAAACAAATCAAACAAAAGTTAATGACAGGAGAAAAACAGCGCAAAAAAGACCATCAAAGCTAAAGCCGGCA
ACTATTGAGGCAATGCTGGTTACAGTGACTGCCGGGTCTAACTTAACTTTGGTTGGACCTAAAGCAGAAGGAAAAGT
TACTTGGTTTGATGGAGATTTAAAAAGACCATGTGAGCCTAATTACAGACTAAGACACGAATGTAATAATCAAAACT
TAACTCTGATTAATGTAACTAAAGATTATGAGGGAACTTACTATGGTACAAATGACAAAGATGAGGGCAAAAGGTAC
AGAGTGAAAGTAAATACTACAAATTCTCAATCTGTGAAAATTCAGCCATATACCAGACAAACTACTCCTGATCAAGA
GCACAAATTTGAATTACAGTTCGAAACTAATGGAAATTATGATTCAAAAATTCCCTCAACCACTGTGGCAATCGTGG
TGGGTGTGATTGCGGGCTTCATAACTCTGATCATTGTCTTCATATGCTACATCTGCTGCCGCAAGCGTCCCAGGGCA
TACAATCATATGGTAGACCCACTACTCAGCTTCTCTTACTAAGACTCAGTCACTTTCATTTCAGAACCATGAAGGCT
TTCACAGCTTGCGTTCTGATTAGCCTAGTCACACTTAGTGTAGCTATTAAAAATCAATATCATGTTCATAATGTTAC
CAGAGATGGATATATCACATTAAATGTAACAATTGATAATACTACCTGGACAAGATATCATTTAAATAAGTGGCATC
AAATTTGTACGTGGTCAGACCCATCATACAAATGTCACAGCAATGGCAGCATTACCATTCATGCTTTCAATATTACT
TCTGGCCAGTACAAAGCTGAAAGTTTTACTAACTGGTTTAGATATTACGGTAATCATAAACATGAAATTCATATTTT
TAACATAACTGTAATTGAGCATCCTACAACAAAAGCACCCACCACTGCTAATACAGCTACATCAATTAAATCAACAA
CCACACAGCCTACTACTAGGGAGACAACTCAACCTACCACCACAGTCAGTACAACTACTGAGACCACTACTCAAACT
ACACAGCTAGACACAACAGTGCAGAATAGCACTGTGTTGGTTAGGTATCTGTTGAGGGAGGAAAGTACTACTGAACA
GACAGAGGCTACCTCAAGTGCCTTTAGCAGCACTGCAAATTTAACTTCGCTTGCTTGGACTAATGAAACCGGAGTAT
CATTGATGAATCATCAGCCTTTCTCAGGTTTGGATATTCAAATTACTTTTCTGGTTGTTTGTGGGATCTTTATTCTT
GTGGTTCTTCTGTACTTTGTCTGCTGCAAAGCCAGAGAGAAATCTAGGAGGCCCATCTACAGGCCAGTAATCGGGGA
ACCTCAGCCACTCCAAGTGGAAGGGGGTCTAAGGAATCTTCTTTTCTCTTTTTCAGTATGGTGATCAGCCATGATTC
CTAGGTTCTTCCTATTTAACATCCTCTTCTGTCTCTTCAACATCTGCGCTGCCTTTGCAGCCGTCTCGCACGCCTCG
CCCGACTGTCTCGGGCCCTTCCCAACCTACCTCCTCTTTGCCCTGCTCACCTGCACCTGCGTCTGCAGCATTGTCTG
CCTGGTCATCACCTTCCTGCAGCTTATCGACTGGTGCTGTGCGCGCTACAATTATCTCCATCACAGTCCCGAATACA
GGGACAAGAACGTAGCCAGAATCTTAAGGCTCATCTGACCATGCAGACTCTGCTCATGCTGCTATCCCTCCTATCCC
CTGCCCTAGCCACTTATGCTGATTACTCTAAATGCAAATTCGCAGACATATGGAATTTCTTAGATTGCTATCAGGAA
AAAATTGATATGCCCTCCTATTACTTGGTGATTGTGGGAATAGTCATGGTCTGCTCCTGCACTTTCTTTGCCATCAT
GATTTACCCCTGTTTTGATCTCGGCTGGAACTCTGTTGAAGCATTCACATACACACTAGAAAGCAGTTCACTAGCCT
CCACGCCACCACCCACACCGCCTCCTCGCAGAAATCAGTTCCCCCTGATACAGTACTTAGAAGAGCCCCCTCCCCGA
CCCCCTTCCACTGTTAGCTACTTTCACATAACCGGCGGCGATGACTGACCACCACCTGGACCTCGAGATGGACGGCC
AGGCCTCCGAGCAGCGCATCCTGCAACTGCGCGTCCGTCAGCAGCAGGAGCGGGCCGCCAAGGAGCTCCTTGATGCC
ATCAACATCCACCAGTGCAAGAAGGGCATCTTCTGCCTGGTCAAACAGGCAAAGATCACCTACGAGCTCGTGTCCAA
CGGCAAACAGCATCGCCTTACCTATGAGATGCCCCAGCAGAAGCAGAAGTTCACCTGCATGGTGGGCGTCAACCCCA
TAGTCATCACCCAGCAGTCGGGCGAGACCAACGGCTGCATCCACTGCTCCTGCGAAAGCCCCGAGTGCATCTACTCC
CTTCTCAAGACCCTTTGCGGACTCCGCGACCTCCTCCCCATGAACTGATGTTGATTAAAAGCCCAGAAACCAATCAG
ACCCTTCCTCATTTCCCCATCCCAATACTCATAAGAATAAATCATTGGAATTAATCATTCAATAAAGATCACTTACT
TGAAATCTGAAAGTATGTCTCTGGTGTAGTTGCTCAGCAACACCTCGGTACCCTCCTCCCAGCTCTGGTACTCCAGT
CCCCGGCGGGCGGCGAACTTCCTCCACACCTTGAAAGGGATGTCAAATTCCTGGTCCACAATTTTCATTGTCTTCCC
TCTTAGATGTCAAAGAGGCTCCGGGTGGAAGATGACTTCAACCCCGTCTACCCCTATGGCTACGCGCGGAATCAGAA
TATCCCCTTCCTCACTCCCCCCTTTGTCTCCTCCGATGGATTCAAAAACTTCCCCCCTGGGGTACTGTCACTCAAAC
TGGCTGATCCAATCACCATTACCAATGGGGATGTATCCCTCAAGGTGGGAGGTGGTCTCACTTTGCAAGATGGAAGC
CTAACTGTAAACCCTAAGGCTCCACTGCAAGTTAATACTGATAAAAAACTTGAGCTTGCATATGATAATCCATTTGA
AAGTAGTGCTAATAAACTTAGTTTAAAAGTAGGACATGGATTAAAAGTATTAGATGAAAAAAGTGCTGCGGGGTTAA
AAGATTTAATTGGCAAACTTGTGGTTTTAACAGGAAAAGGAATAGGCACTGAAAATTTAGAAAATACAGATGGTAGC
AGCAGAGGAATTGGTATAAATGTAAGAGCAAGAGAAGGGTTGACATTTGACAATGATGGATACTTGGTAGCATGGAA
CCCAAAGTATGACACGCGCACACTTTGGACAACACCAGACACATCTCCAAACTGCACAATTGCTCAAGATAAGGACT
CTAAACTCACTTTGGTACTTACAAAGTGTGGAAGTCAAATATTAGCTAATGTGTCTTTGATTGTGGTCGCAGGAAAG
TACCACATCATAAATAATAAGACAAATCCAAAAATAAAAAGTTTTACTATTAAACTGCTATTTAATAAGAACGGAGT
GCTTTTAGACAACTCAAATCTTGGAAAAGCTTATTGGAACTTTAGAAGTGGAAATTCCAATGTTTCGACAGCTTATG
AAAAAGCAATTGGTTTTATGCCTAATTTGGTAGCGTATCCAAAACCCAGTAATTCTAAAAAATATGCAAGAGACATA
GTTTATGGAACTATATATCTTGGTGGAAAACCTGATCAGCCAGCAGTCATTAAAACTACCTTTAACCAAGAAACTGG
ATGTGAATACTCTATCACATTTAACTTTAGTTGGTCCAAAACCTATGAAAATGTTGAATTTGAAACCACCTCTTTTA
CCTTCTCCTATATTGCCCAAGAATGAAAGACCAATAAACGTGTTTTTCATTTGAAATTTTCATGTATCTTTATTGAT
TTTTACACCAGCACGAGTAGACAGTCTCCCACCACCAGCCCATTTTACAGTGTACACGGTTCTCTCAGCACGGGTAG
CCTTAAATAGGGAAATATTCTCATTAGTGCGGGAATTGGACTTGGGGTCTATAATCCACACAGTTTCCTGGCGAGCC
AAACGGGGGTCGGTGATTGAAATAAAGCCGTCCTCTGAAAAGTCATCCAAGCGGGCCTCACAGTCCAAGGTCACAGT
CTGGTGGAACGAGAAGAACGCACAGATTCATACTCGGAAAACAGGATGGGTCTGTGCCTCTCCATCAGCGCCCTCAG
CAGTCTCTGCCGCCGGGGCTCGGTGCGGCTGCTGCAAATGGGATCGGGATCACAAGTCTCTCTGACTATGATCCCAA
CAGCCTTCAGCATCAGTCTCCTGGTGCGACGGGCACAGCACCGCATCCTGATCTCTGCCATGTTCTCACAGTAAGTG
CAGCACATAATCACCATGTTATTCAGCAGCCCATAATTCAGGGCGCTCCAGCCAAAGCTCATGTTGGGAATGATGGA
ACCCACGTGACCATCGTACCAGATGCGACAGTATATCAGGTGCCTGCCCCTCATGAACACACTGCCCATGTACATGA
TCTCTTTGGGCATGTTTCTGTTTACAATCTGGCGGTACCAGGGGAAGCGCTGGTTGAACATGCACCCGTAAATGACT
CTCCTGAACCACACGGCCAGCAGGGTGCCTCCCGCCCGACACTGCAGGGAGCCAGGGGATGAACAGTGGCAATGCAG
GATCCAGCGCTCGTACCCGCTCACCATTTGAGCTCTTACCAAGTCCAGGGTAGCGGGGCACAGGCACACTGACATAC
ATCTTTTTAAAATTTTTATTTCCTCTGTGGTGAGGATCATATCCCAGGGGACTGGAAACTCTTGGAGCAGGGTAAAG
CCAGCAGCACATGGTAATCCACGGACAGAACTTACATTATGATAATCTGCATGATCACAATCGGGCAACAGGGGATG
TTGTTCAGTCAGTGAAGCCCTGGTTTCCTCATCAGATCGTGGTAAACGGGCCCTGCGATATGGATGATGGCGGAGCG
AGCTGGATTGAATCTCGGTTTGCATTGTAGTGGATTCTCTTGCGTACCTTGTCGTACTTCTGCCAGCAGAAATGGGC
CCTTGAACAGCATATACCCCTCCTACGGCCGTCCTTTCGCTGCTGCCGCTCAGTCATCCAACTAAAGTACATCCATT
CTCGAAGATTCTGGAGAAGTTCCTCTGCATCTGATAAAATAAAAAACCCGTCCATGCGAATTCCCCTCATCACATCA
GCCAGGACTCTGTAGGCCATCCCCATCCAGTTAATGCTGCCTTGTCTATCATTCAGAGGGGGCGGTGGCAGGACTGG
AAGAACCATTTTTATTCCAAACGGTCTCGAAGGACGATAAAGTGCAAGTCACGCAGGTGACAGCGTTCCCCTCCGCT
GTGCTGGTGGAAACAGACAGCCAGGTCAAAACCCACTCTATTTTCAAGGTGCTCGACCGTGGCTTCGAGCAGTGGCT
CTACGCGCACATCCAGCATAAGAATCACATTAAAGGCTGGCCCTCCATCGATTTCATCAATCATCAGGTTACATTCC
TGCACCATCCCCAGGTAATTCTCATTTTTCCAGCCTTGGATTATCTCTACAAATTGTTGGTGTAAGTCCACTCCGCA
CATGTGGAAAAGCTCCCACAGTGCCCCCTCCACTTTCATAATCAGGCAGACCTTCATAATAGAAACAGATCCTGCTG
CTCCACCACCTGCAGCGTGTTCAAAACAACAAGATTCAATAAGGTTCTGCCCTCCGCCCTGAGCTCGCGCCTCAATG
TCAGCTGCAAAAAGTCACTTAAGTCCTGGGCCACTACAGCTGACAATTCAGAGCCAGGGCTAAGCGTGGGACTGGCA
AGCGTAAGGGAAAACTTTAATGCTCCAAAGCTAGCACCCAAAAACTGCATGCTGGAATAAGCTCTCTTTGTGTCTCC
GGTGATGCCTTCCAAAATGTGAGTGATAAAGCGTGGTAGTTTTTCTTTAATCATTTGCGTAATAGAAAAGTCCTCTA
AATAAGTCACTAGGACCCCAGGGACCACAATGTGGTAGCTTACACCGCGTCGCTGAAGCATGGTTAGTAGAGATGAG
AGTCTGAAAAACAGAAAGCATGCACTAAACTAAGGTGGCTATTTTCACTGAAGGAAAAATCACTCTCTCCAGCAGCA
GGGTACCCACTGGGTGGCCCTTGCGGACATACAAAAATCGGTCCGTGTGATTAAAAAGCAGCACAGTAAGTTCCTGT
CTTCTTCCGGCAAAAATCACATCAGACTGGGTTAGTATGTCCCTGGCATGGTAGTCATTCAAGGCCATAAATCTGCC
CTGATATCCAGTAGGAACCAGCACACTCACTTTTAGGTGAAGCAATACCACCCCATGCGGAGGAATGTGGAAAGATT
CAGGGCAAAAAAATTATATCTATTGCTAGCCCCTTCCTGGACGGGAGCAATCCCTCCAGGACTATCTATAAAAGCAT
ACAGAGATTCAGCCATAGCTTAGCCCGCTTACCAGTAGACAGAAAGCACAGCAGTACAAGCGCCAACAGCAGCAACT
GACTACCCACTGACCCAGCTCCCTATTTAAAGGCACCTTACACTGACGTAATGACCAAAGGTCTAAAAACCCCGCCA
AAAAAAACACACACGCCCTGGGTGTTTTTCACAAAAACACTTCCGCGTTCTCACTTCCTCGTATCGATTTTGTGACT
CAACTTCCGGGTTCCCACGTTACGTCACTTCTGCCCTTACATGTAACTTGGCCGTATGGCGCCATCTTGCCCACGTC
CAAAATGGCTTTCATGACCGGCCACGCCTCCGCGCCGGCCGTTAGCCGTGCGTCGTGACGTTATTTGCATCACCGCT
TCTCGTCCAATCAGCGTTGGCTCCGCCCCAAAACCGTTAAAATTCAAAAGCTCATTTGCATATTAACTTTTGTTTAC
TTTGTGGGGTATATTATGATGATG

GenBank Accession No. ABK59080 (SEQ ID NO: 235)

MSKRLRVEDDFNPVYPYGYARNQNIPFLTPPFVSSDGFKNFPPGVLSLKL
ADPITITNGDVSLKVGGGLTLQDGSLTVNPKAPLQVNTDKKLELAYDNPF
ESSANKLSLKVGHGLKVLDEKSAAGLKDLIGKLVVLIGKGIGTENLENTD
GSSRGIGINVRAREGLIFDNDGYLVAWNPKYDTRTLWTTPDTSPNCTIAQ
DKDSKLTLVLTKCGSQILANVSLIVVAGKYHIINNKTNPKIKSFTIKLLF
NKNGVLLDNSNLGKAYWNFRSGNSNVSTAYEKAIGEMPNLVAYPKPSNSK
KYARDIVYGTIYLGGKPDQPAVIKTTFNQETGCEYSITENFSWSKTYENV
EFETTSFTFSYIAQE

GenBank Accession No. ABK59086 (SEQ ID NO: 236)

MRRAVVSSSPPPSYESVMAQATLEVPFVPPRYMAPTEGRNSIRYSELAPL
YDTTRVYLVDNKSADIASLNYQNDHSNFLTTVVQNNDFTPAEASTQTINF
DERSRWGGDLKTILHTNMPNVNEYMFTSKFKARVMVARKKAEGADANDRS
KDILEYQWFEFTLPEGNFSETMTIDLMNNAILENYLQVGRQNGVLESDIG
VKFDSRNFKLGWDPVTKLVMPGVYTYEAFHPDVVLLPGCGVDFTESRLSN
LLGIRKKQPFQEGFRIMYEDLVGGNIPALLNVKEYLKDKEEAGKADANTI
KAQNDAVPRGDNYASAAEAKAAGKEIELKAILKDDSDRSYNVIEGTTDTL
YRSWYLSYTYGDPEKGVQSWTLLTTPDVTCGAEQVYWSLPDLMQDPVTFR
STQQVSNYPVVGAELMPFRAKSFYNDLAVYSQLIRSYTSLTHVFNRFPDN
QILCRPPAPTITTVSENVPALTDHGTLPLRSSIRGVQRVTVTDARRRTCP
YVYKALGIVAPRVLSSRTF

GenBank Accession No. ABK59070 (SEQ ID NO: 237)

MCLTARERAKMATPSMMPQWAYMHIAGQDASEYLSPGLVQFARATDTYFS
LGNKERNPTVAPTHDVTTDRSQRLTLRFVPVDREDTTYSYKARFTLAVGD
NRVLDMASTYFDIRGVLDRGPSFKPYSGTAYNSLAPKGAPNPSQWTTKEK
QNGGTGAEKDVTKTFGLAAMGGSNISKDGLQIGTDKTANAEKPIYADKTF
QPEPQVGEENWQDNDEYYGGRALKKDTKMKPCYGSFAKPTNKEGGQAKLK
ETPNGTDPQYDVDMAFFDSSTINIPDVVLYTENVDLETPDTHVVYKPGKE
DDSSEANLTQQSMPNRPNYIGFRDNFVGLLYYNSTGNMGVLAGQASQLNA
VVDLQDRNTELSYQLLLDSLGDRTRYFSMWNSAVDSYDPDVRIIENHGVE
DELPNYCFPLDGVQTNSAYQGVKLKPDQTGGGVNGDWVKDDDISAHNQIG
KGNIFAMEINLQANLWKSFLYSNVALYLPDSYKYTPANVTLPANTNTYEY
MNGRVVAPSLVDAYINIGARWSLDPMDNVNPFNHHRNAGLRYRSMLLGNG
RYVPFHIQVPQKFFAIKNLLLLPGSYTYEWNFRKDVNMILQSSLGNDLRV
DGASVREDSVNLYATFFPMAHNTASTLEAMLRNDTNDQSENDYLSAANML
YPIPAKATNVPISIPSRNWAAFRGWSFTRLKTKETPSLGSGEDPYFVYSG
SIPYLDGTFYLNHTFKKVSIMFDSSVSWPGNDRLLTPNEFEIKRSVDGEG
YNVAQCNMTKDWFLVQMLSHYNIGYQGFHVPEGYKDRMYSFFRNFQPMSR
QVVDEINYKDYKAVTLPFQHNNSGFTGYLAPTMRQGQPYPANFPYPLIGS
TAVPSVTQKKFLCDRVMWRIPFSSNFMSMGALTDLGQNMLYANSAHALDM
TFEVDPMDEPTLLYLLFEVEDVVRVHQPHRGVIEAVYLRTPFSAGNATT

GenBank Accession No. AY737798 SEQ ID NO: 207)

CAATCAATATAATATACCTTATAGATGGAATGGTGCCAATATGTAAATGAGGTAATTTAAAAAAGTGCGCGCTGTGT
GGTGATTGGCTGCGGGGTGAACGGCTAAAAGGGGCGGACATGCTGGGAGGTGACGTGACTTATGGGGGAGGAGTTAT
GTTGCAAGTTATCGCGGTAAAGGTGACGTAAAACGAGGTGTGGTTTGGACACGGAAGTAGACAGTTTTCCCACGCTT
ACTGACAGGATATGAGGTAGTTTTGGGCGGATGCAAGTGAAAATTCTCCATTTTCGCGCGAAAACTGAATGAGGAAG
TGAATTTCTGAGTCATTTCGCGGTTATGACAGGGTGGAGTATTTGCCGAGGGCCGAGTAGACTTTGACCGTTTACGT
GGAGGTTTCGATTACCGTGTTTTTCACCTAAATTTCCGCGTACGGTGTCAAAGTCCTGTGTTTTTACGTAGGTGTCA
GCTGATCGCTAGGGTATTTAAACCTGTCGAGTTCCGTCAAGAGGCCACTCTTGAGTGCCAGCGAGAAGAGTTTTCTC
CTCCGCGCCGCGAGTCAGTTTTGCGCTTTGAAAATGAGACACCTGCGATTCCTGCCACAGGAGATTATCTCCAGCGA
GACCGGGATAGAAATACTGGAGTTTGTGGTAAATACCCTGATGGGAGATGACCCGGAACCGCCAGCGCAGCCTTTCG
ATCCACCTACGCTGCACGATCTGTATGATTTAGAGGTAGACGGGCCGGACGATCCCAATGAGGAAGCTGTAAATGGG
TTTTTTACTGATTCTATGCTACTAGCTGCCGATGAAGGATTGGACATAAACCCTCCTCCTGAGACCCTTGATACCCC
AGGGGTGGTTGTGGAAAGCGGCAGAGGTGGGATAAAATTGCCTGATCTGGGAGCAGCTGAAATGGACTTGCGTTGTT
ATGAAGAGGGTTTTCCTCCGAGTGATGATGAAGATGGGGAAACTGAACAGTCCATCCATACCGCAGTGAATGAGGGA
GTAAAAGCTGCCAGCGATGTTTTTAAGTTGGACTGTCCGGAGCTGCCTGGACATGGCTGTAAGTCTTGTGAATTTCA
CAGGAATAACACTGGAATGAAAGAACTATTGTGCTCGCTTTGCTATATGAGAATGCACTGCCATTTTATTTACAGTA
AGTGTATTTAAGTGAAATTTAAAGGAATAGTGTAGCTGTTTAATAACTGTTGAATGGTAGATTTATGTTTTTACTTG
TGATTTTTTGTAGGTCCTGTGTCTGATGATGAGTCGCCTTCTCCTGATTCAACTACCTCACCTCCTGAAATTCAGGC
GCCCGTACCTGCAAACGTATGCAAGCCCATTCCTGTGAAGCCTAAGTCTGGGAAACGCCCTGCTGTGGATAAGCTTG
AGGACTTGTTGGAGGGTGGGGATGGACCTTTGGACCTTAGTACCCGGAAACTGCCAAGGCAATGAGTGCCCTGCAGC
TGTGTTTATTTAATGTGACGTCATGTAATAAAATTATGTCAGCTGCTGAGTGTTTTATTGCTTCTTGGGTGGGGACT
TGGATATATAAGTAGGAGCAGATCTGTGTGGTTAGCTTATAGCAACCTGCTGCCATCCATGGAGGTTTGGGCTATCT
TGGAAGACCTGAGACAGACTAGGCTACTGCTAGAAAACGCCTCGGACGGAGTCTCTGGCTTTTGGAGATTCTGGTTC
GGTGGTGATCTAGCTAGGCTAGTGTTTAGGATAAAACAGGACTACAGGGAAGAATTTGAAAAGTTATTGGACGACAG
TCCAGGACTTTTTGAAGCTCTTAACTTGGGCCATCAGGCTCATTTTAAGGAGAAGGTTTTATCAGTTTTAGATTTTT
CTACTCCTGGTAGAACTGCTGCTGCTGTAGCTTTTCTTACTTTTATATTGGATAAATGGATCCGACAAACCCACTTC
AGCAAGGGATACGTTTTGGATTTCATAGCAGCAGCTTTGTGGAGAACATGGAAGGCTCGCAGCATGAGGACAATCTT
AGATTACTGGCCAGTGCAGCCTCTGGGAGTAGCAGGGATACTGAGACACCCACCGACCATGCCAGCGGTTCTGGAGG
AGGAGCAGCAGGAGGACAATCCGAGAGCCGGCCTGGACCCTCCGGTGGAGGAGTAGCTGACCTGTTTCCTGAACTGC
GACGGGTGCTTACTAGGTCTACGTCCAGTGGACAGGACAGGGGCATTAAGAGGGAAAGGAATCCTAGTGGGAATAAT
TCAAGAACCGAGTTGGCTTTAAGTTTAATGAGCCGTAGGCGTCCTGAAACTGTTTGGTGGCATGAGGTTCAGAGCGA
AGGCAGGGATGAAGTTTCAATATTGCAGGAGAAATATTCACTAGAACAACTTAAGACCTGTTGGTTGGAACCTGAGG
ATGATTGGGAGGTGGCCATTAGGAATTATGCTAAGATATCTCTGAGGCCTGATAAACAGTATAGAATTACTAAAAAG
ATTAATATTAGAAATGCATGCTACATATCAGGGAATGGGGCAGAGGTTATAATAGATACCCAAGATAAAGCAGCTTT
TAGATGTTGTATGATGGGTATGTGGCCAGGGGTTGTCGGCATGGAAGCAGTAACATTTATGAATATTAGGTTTAAAG
GGGATGGGTATAATGGCATTGTATTTATGGCTAACACTAAGCTGATTCTACATGGTTGTAGCTTTTTTGGGTTTAAT
AATACTTGTGTAGAAGCTTGGGGGCAAGTTGGTGTGAGGGGTTGTAGTTTTTATGCATGCTGGATTGCAACATCAGG
TAGGGTCAAGAGTCAGTIGTCTGTGAAGAAATGCATGTTTGAGAGATGTAATCTTGGCATACTGAATGAAGGTGAAG
CAAGGGTCCGCCACTGCGCAGCTACAGAAACTGGCTGCTTCATTCTAATAAAGGGAAATGCCAGTGTGAAGCATAAT
ATGATCTGTGGACATTCGAATGAGAGGCCTTATCAGATGCTGACTTGCGCTGGTGGACATTGCAATATTCTTGCTAC
CGTGCATATCGTTTCCCATGCACGCAAGAAATGGCCTGTATTTGAACATAATGTGATTACCAAGTGCACCATGCACA
TAGGTGGTCGCAGGGGAATGTTTATGCCTTACCAGTGTAACATGAATCATGTGAAGGTGATGTTGGAACCAGATGCC
TTTTCCAGAGTGAGCTTAACAGGAATCTTTGATATGAATATTCAACTATGGAAGATACTGAGATATGATGACACTAA
ACCGAGGGTGCGCGCATGCGAATGCGGAGGCAAGCATGCTAGATTCCAGCCGGTGTGCGTGGATGTGACTGAAGACC
TGAGACCCGATCATTTGGTGCTTGCCTGCACTGGAGCGGAGTTCGGTTCTAGTGGTGAAGAAACTGACTAAAGTGAG
TAGTGGGGCAAGATGTGGATGGAGACTTTCAGGTTGGTAAGGTGGACAGATTGGGTAAATTTTGTTAATTTCTGTCT
TGCAGCTGCCATGAGTGGAAGCGCTTCTTTTGAGGGGGGAGTATTTAGCCCTTATCTGACGGGCAGGCTCCCATCAT
GGGCAGGAGTTCGTCAGAATGTCATGGGATCCACTGTGGATGGGAGACCCGTCCAGCCCGCCAATTCCTCAACGCTG
ACCTATGCCACTTTGAGTTCGTCACCATTGGATGCAGCTGCAGCCGCCGCCGCTACTGCTGCCGCCAACACCATCCT
TGGAATGGGCTATTACGGAAGCATCGTTGCCAATTCCAGTTCCTCTAATAACCCTTCAACCCTGGCTGAGGACAAGC
TACTTGTTCTCTTGGCTCAGCTCGAGGCCTTAACCCAACGCTTAGGCGAACTGTCTAAGCAGGTGGCCCAGTTGCGT
GAGCAAACTGAGTCTGCTGTTGCCACAGCAAAGTCTAAATAAAGATCTCAAATCAATAAATAAAGAAATACTTGTTA
TAAAAACAAATGAATGTTTATTTGGTTTTTCGCGCGCGGTATGCCCTGGACCATCGGTCTCGATCATTGAGAACTCG
GTGGATCTTTTCCAGTACCCTGTAAAGGTGGGATTGAATGTTTAGATACATGGGCATTAGTCCGTCTCGGGGGTGGA
GATAGCTCCATTGAAGAGCCTCTTGCTCCGGGGTAGTGTTATAAATCACCCAGTCATAGCAAGGTCGGAGTGCATGG
TGTTGCACAATATCTTTTAGGAGCAGACTAATTGCAACGGGGAGGCCCTTAGTGTAGGTGTTTACAAATCTGTTGAG
CTGGGACGGGTGCATCCTGGGGGAAATTATATGCATTTTGGACTGGATCTTGAGGTTGGCAATGTTGCCGCCTAGAT
CCCGTCTCGGGTTCATATTGTGCAGAACCACCAAGACAGTGTATCCGGTGCACTTGGGAAATTTATCATGCAGCTTA
GAGGGAAAAGCATGAAAAAATTTGGAGACGCCTTTGTGACCCCCCAGATTCTCCATGCACTCATCCATAATGATAGC
GATGGGGCCGTGGGCAGCGGCACGGGCGAACACGTTCCGGGGGTCTGAAACATCATAGTTATGCTCCTGAGTCAGGT
CATCATAAGCCATTTTAATAAACTTTGGGCGGAGGGTGCCAGATTGGGGGATGAAAGTTCCCTCTGGCCCGGGAGCA
TAGTTTCCCTCACATATTTGCATTTCCCAGGCTTTCAGTTCCGAGGGGGGGATCATGTCCACCTGCGGGGCTATAAA
AAATACCGTTTCTGGAGCCGGGGTGATTAACTGGGATGAGAGCAAATTCCTAAGCAGCTGAGACTTGCCGCACCCAG
TGGGACCGTAAATGACCCCAATTACGGGTTGCAGATGGTAGTTTAGGGAGCGACAGCTGCCGTCCTCCCGGAGCAGG
GGGGCCACTTCGTTCATCATTTCCCTTACATGGATATTTTCCCGCACCAAGTCCGTTAGGAGGCGCTCTCCCCCAAG
GGATAGAAGCTCCTGGAGCGAGGAGAAGTTTTTCAACGGTTTCAGCCCGTCAGCCATGGGCATTTTGGAAAGAGTCT
GTTGCAAGAGCTCGAGCCGGTCCCAGAGCTCGGTGATGTGCTCTATGGCATCTCGATCCAGCAGACCTCCTCGTTTC
GCGGGTTGGGACGGCTCCTGGAGTAGGGAATCAGACGATGAGCGTCCAGCGCTGCTAGGGTCCGATCCTTCCATGGT
CGCAGCGTCCGAGTCAGGGTTGTTTCCGTCACGGTGAAGGGGTGCGCGCCTGGTTGGGCGCTTGCGAGGGTGCGCTT
CAGACTCATCCTGCTGGTCGAGAACCGCTGCCGATCGGCGCCCTGCATGTCGGCCAGGTAGCAGTTTACCATGAGTT
CGTAGITGAGCGCCTCGGCCGCGTGGCCTTTGGCACGGAGCTTACCTTTGGAAGTTTTATGGCAGGCGGGGCAGTAG
ATACATTTGAGGGCATACAGCTTGGGCGCGAGGAAAATGGATTCGGGGGAGTATGCATCCGCACCGCAGGAGGCGCA
GACGGTTTCGCACTCCACGAGCCAGGTCAGATCCGGCTCATCGGGGTCAAAAACAAGTTTTCCGCCATGTTTTTTGA
TGCGTTTCTTACCTTTGGTTTCCATGAGTTCGTGTCCCCGCTGGGTGACAAAGAGGCTGTCCGTGTCCCCGTAGACC
GACTTTATGGGCCTGTCCTCGAGCGGAGTGCCTCGGTCCTCTTCGTAGAGGAACCCAGCCCACTCTGATACAAAAGC
GCGTGTCCAGGCCAGCACAAAGGAGGCCACGTGGGAGGGGTAGCGGTCGTTGTCAACCAGGGGGTCCACCTTCTCTA
CGGTATGTAAACACATGTCCCCCTCCTCCACATCCAAGAATGTGATTGGCTTGTAAGTGTAGGCCACGTGACCAGGG
GTCCCCGCCGGGGGGGTATAAAAGTGGGCGGGCCTCTGTTCGTCCTCACTGTCTTCCGGATCACTGTCCAGGAGCGC
CAGCTGTTGGGGTAGGTATTCTCTCTCGAAGGCGGGCATGACCTCTGCACTCAGGTTGTCAGTTTCTAGGAACGAGG
AGGATTTGATATTGACAGTACCAGCAGAGATGCCTTTCATAAGACTCTCGTCCATCTGGTCAGAAAACACAATCTTC
TTGTTATCCAGCTTGGTGGCAAATGATCCATAGAGGGCATTGGATAGAAGCTTGGCGATGGAGCGCATGGTTTGGTT
CTTTTCCTTGTCCGCGCGCTCCTTGGCGGCGATGTTAAGCTGGACGTACTCGCGCGCCACACATTTCCATTCAGGGA
AGATGGTTGTCAGTTCATCCGGAACTATTCTGACTCGCCATCCCCTATTGTGCAGGGTTATCAGATCCACACTGGTG
GCTACCTCGCCTCGGAGGGGCTCATTGGTCCAGCAGAGTCGACCTCCTTTTCTTGAACAGAAAGGGGGGAGGGGGTC
TAGCATGAACTCATCAGGGGGGTCCGCATCTATGGTAAATATTCCCGGTAGCAAATCTTTGTCAAAATAGCTGATGG
TGGCGGGATCATCCAAGGTCATCTGCCATTCTCGAACTGCCAGCGCGCGCTCGTAGGGGTTAAGAGGGGTGCCCCAG
GGCATGGGGTGGGTGAGCGCGGAGGCATACATGCCACAGATATCGTAGACATAGAGGGGCTCTTCGAGGATGCCGAT
GTAAGTGGGATAACAGCGCCCCCCTCTGATGCTTGCTCGCACATAGTCATAGAGTTCATGTGAGGGGGCGAGAAGAC
CCGGGCCCAGATTGGTGCGGTTGGGTTTTTCCGCCCTGTAAACGATCTGGCGAAAGATGGCATGGGAATTGGAAGAG
ATAGTAGGTCTCTGGAATATGTTAAAATGGGCATGAGGTAGGCCTACAGAGTCCCTTATGAAGTGGGCATATGACTC
TTGCAGCTTGGCTACCAGCTCGGCGGTGACGAGTACGTCCAGGGCACAGTAGTCGAGAGTTTCCTGGATGATGTCAT
AACGCGGTTGGCTTTTCTTTTCCCACAGCTCGCGGTTGAGAAGGTATTCTTCGCGATCCTTCCAGTACTCTTCGAGG
GGAAACCCGTCTTTTTCTGCACGGTAAGAGCCCAACATGTAGAACTGATTGACTGCCTTGTAGGGACAGCATCCCTT
CTCCACTGGGAGAGAGTATGCTTGGGCTGCATTGCGCAGCGAGGTATGAGTGAGGGCAAAAGTGTCCCTGACCATGA
CTTTGAGGAATTGATACTTGAAGTCGATGTCATCACAGGCCCCCTGTTCCCAGAGTTGGAAGTCCACCCGCTTCTTG
TAGGCGGGGTTGGGCAAAGCGAAAGTAACATCATTGAAGAGGATCTTGCCGGCCCTGGGCATGAAATTTCGGGTGAT
TCTGAAAGGCTGAGGGACCTCTGCTCGGTTATTGATAACCTGAGCGGCCAAGACGATCTCATCAAAGCCATTGATGT
TGTGCCCCACTATGTACAGTTCTAAGAATCGAGGGGTGCCCCTGACATGAGGCAGCTTCTTGAGTTCTTCAAAAGTG
AGGTCTGTAGGGTCAGTGAGAGCATAGTGTTCGAGGGCCCATTCGTGCACGTGAGGGTTCGCTTTGAGGAAGGAGGA
CCAGAGGTCCACTGCCAGTGCTGTTTGTAACTGGTCCCGGTACTGACGAAAATGCTGCCCGACTGCCATCTTTTCTG
GGGTGACGCAATAGAAGGTTTGGGGGTCCTGCCGCCAGCGATCCCACTTGAGTTTTATGGCCAGGTCATAGGCGATG
TTGACGAGCCGCTGGTCTCCAGAGAGTTTCATGACCAGCATGAAGGGGATTAGCTGCTTGCCAAAGGACCCCATCCA
GGTGTAGGTTTCCACATCGTAGGTGAGGAAGAGCCTTTCTGTGCGAGGATGAGAGCCAATCGGGAAGAACTGGATCT
CCTGCCACCAGTTGGAGGAATGGCTGTTGATGTGATGGAAGTAGAACTCCCTGCGACGCGCCGAGCATTCATGCTTG
TGCTTGTACAGACGGCCGCAGTACTCGCATCGATTCACGGGATGCACCTCATGAATGAGTTGTACCTGACTTCCTTT
GACGAGAAATTTCAGTGGAAAATTGAGGCCTGGCGATTGTACCTCGCGCTCTACTATGTTGTCTGCATCGGCATGAC
CATCTTCTGTCTCGATGGTGGTCATGCTGACGAGCCCTCGCGGGAGGCAAGTCCAGACCTCGGCGCGGCAGGGGCGG
AGCTCGAGGACGAGAGCGCGCAGGCCGGAGCTGTCCAGGGTCCTGAGACGCTGCGGAGTCAGGTTAGTAGGCAGTGT
CAGGAGATTGACTTGCATGATCTTTTCGAGGGCGTGAGGGAGGTTCAGATGGTACTTGATCTCCACGGGTCCGTTGG
TGGAGATGTCGATGGCTTGCAGGGTTCCGTGCCCCTTGGGCGCTACCACCGTGCCCTTGTTTTTCCTTTTGGGCGGC
GGTGGCTCTGTTGCTTCTTGCATGTTTAGAAGCGGTGTCGAGGGCGCGCACCGGGCGGCAGGGGCGGTTCGGGACCC
GGCGGCATGGCCGGCAGTGGTACGTCGGCGCCGCGCGCGGGTAGGTTCTGGTACTGCGCCCTCAGAAGACTCGCATG
CGCCACGACGCGGCGGTTGACATCCTGGATCTGACGCCTCTGGGTGAAAGCTACCGGCCCCGTGAGCTTGAACCTGA
AAGAGAGTTCAACAGAATCAATCTCGGTATCGTTGACGGCGGCTTGCCTAAGGATTTCTTGCACGTCGCCAGAGTTG
TCCTGGTAGGCGATCTCGGCCATGAACTGCTCGATCTCTTCCTCTTGAAGATCTCCGCGGCCCGCTCTCTCGACGGT
GGCCGCGAGGTCGTTGGAGATACGCCCAATGAGTTGAGAGAATGCATTCATGCCCGCCTCGTTCCAGACGCGGCTGT
AGACCACAGCCCCCACGGGATCTCTCGCGCGCATGACCACCTGGGCGAGGTTGAGCTCCACGTGGCGGGTGAAGACC
GCATAGTTGCATAGGCGCTGGAAAAGGTAGTTGAGTGTGGTGGCGATGTGCTCGGTGACGAAGAAATACATGATCCA
TCGTCTCAGCGGCATCTCGCTGACATCGCCCAGCGCTTCCAAGCGCTCCATGGCCTCGTAGAAGTCCACGGCAAAGT
TGAAAAACTGGGAGTTACGCGCGGACACGGTCAACTCTTCTTCCAAAAGACGGATGAGTTCGGCGATGGTGGTGCGC
ACCTCGAGCTCGAAAGCCCCTGGGATTTCTTCCTCAATCTCTTCTTCTTCCACTAACATCTCTTCCTCTTCAGGTGG
GGCTGCAGGAGGAGGGGGAACGCGGCGACGCCGGCGGCGCACGGGCAGACGGTCGATGAATCTTTCAATGACCTCTC
CGCGGCGGCGGCGCATGGTCTCGGTGACGGCACGACCGTTCTCCCTGGGTCTCAGAGTGAAGACGCCTCCGCGCATC
TCCCTGAAGTGGTGACTGGGAGGCTCTCCGTTGGGCAGGGACACCGCGCTGATTATGCATTTTATTAATTGCCCCGT
AGGTACTCCGCGCAAGGACCTGATCGTCTCAAGATCCACGGGATCTGAAAACCTTTCGACGAAAGCGTCTAACCAGT
CGCAATCGCAAGGTAGGCTGAGCACTGTTTCTTGCGGGCGGGGGCGGCTAGACGCTCGGTCGGGGTTCTCTCTTTCT
TCTCCTTCCTCCTCTTTGGAGGGTGAGACGATGCTGCTGGTGATGAAATTAAAATAGGCAGTTTTGAGACGGCGGAT
GGTGGCGAGGAGCACCAGGTCTTTGGGTCCGGCTTGTTGGATGCGCAGGCGATGGGCCATTCCCCAAGCATTATCCT
GACATCTGGCCAGATCTTTATAGTAGTCTTGCATGAGTCGTTCCACGGGCACTTCTTCTTCGCCCGCTCTGCCATGC
ATGCGAGTGATCCCGAACCCGCGCATGGGCTGGACAAGTGCCAGGTCCGCTACAACCCTTTCTGCGAGGATGGCTTG
CTGCACCTGGGTGAGGGTGGCTTGGAAGTCGTCAAAGTCCACGAAGCGGTGGTAAGCCCCGGTGTTGATTGTGTAGG
AGCAGTTGGCCATGACTGACCAGTTGACTGTCTGGTGCCCAGGGCGCACAAGCTCGGTATACTTAAGGCGCGAGTAT
GCGCGGGTGTCAAAGATGTAATCGTTACAGGTGCGCACCAGGTACTGGTAGCCGATGAGAAAGTGCGGCGGCGGCTG
GCGGTATAGGGGCCATCGCTCTGTAGCCGGGGCGCCAGGGGCGAGGTCTTCCAGCATGAGGCGGTGATAACCGTAGA
TGTACCTGGACATCCAGGTGATACCGGAGGCGGTGGTGGATGCCCGCGGGAACTCGCGTACGCGGTTCCAGATGTTG
CGCAGCGGCATGAAGTAGTTCATGGTAGGCACGGTTTGGCCCGTGAGACGTGCACAGTCGTTGATGCTCTAGACATA
CGGGCAAAAACGAAAGCGGTCAGCGGCTCGTCTCCGTGGCCTGGAGGCTAAGCGAACGGGTTGGGCTGCGCGTGTAC
CCCGGTTCGAATCTCGGATCAGGCTGGAGCCGCAGCTAACGTGGTACTGGCACTCCCGTCTCGACCCAGGCCTGCAC
AAAACCTCCAGGATACGGAGGCGGGTCGTTTTTTTTTTTTTGGCTTTTTCCTGGATGGGAGCCAATGCTGCGTCAAG
CTTTAGAACACTCAGTTCTCGGGGCTGGGAGTGGCTCGCGCCCGTAGTCTGGAGAATCAATCGCCAGGGTTGCGTTG
CGGTGTGCCCCGGTTCGAGTCTTAGCGCGCCGGATCGGCCGGTTTCCGCGACAAGCGAGGGTTTGGCAGCCTCGTCA
TTTCTAAGACCCCGCCAGCCGACTTCTCCAGTTTACGGGAGCGAGCCCTCTTTTTTTGTTTTTTTGTTGCCCAGATG
CATCCCGTGCTGCGACAGATGCGCCCCCAGCAACAGCCCCCTTCTCAGCAGCAGCTACAACAACAGCCACAAAAGGC
TCTTCCTGCTCCTGTAACTACTGCGGCTGCAGCCGTCAGCGGCGCGGGGCAGCCCGCCTATGATCTGGACTTGGAAG
AGGGCGAGGGACTGGCGCGCCTGGGCGCACCATCGCCCGAGCGGCACCCGCGGGTGCAACTGAAAAAGGACTCTCGC
GAGGCGTACGTGCCCCAGCAGAACCTGTTCAGGGACAGGAGCGGCGAGGAGCCTGAGGAAATGCGAGCTTCCCGCTT
TAACGCGGGTCGCGAACTGCGTCACGGTCTGGACCGAAGACGGGTGCTGCGTGATGATGATTTTGAAGTCGATGAAG
TGACAGGAATAAGTCCTGCTAGGGCACATGTGGCCGCGGCCAACCTAGTATCAGCTTACGAGCAGACCGTGAAGGAG
GAGCGCAACTTTCAAAAATCTTTCAACAACCATGTGCGCACCCTGATTGCCCGCGAGGAAGTGACACTGGGTCTGAT
GCACCTGTGGGACCTGATGGAAGCCATTACCCAGAACCCCACCAGCAAACCTCTAACCGCTCAGCTGTTTCTGGTGG
TGCAACATAGTAGAGACAATGAGGCATTTAGGGAGGCGCTGTTGAACATTACTGAGCCCGAGGGGAGATGGTTGTAT
GATCTTATCAATATTCTGCAAAGTATAATCGTGCAAGAACGTAGCCTGGGTCTAGCTGAGAAGGTGGCTGCTATTAA
CTACTCGGTCTTGAGCCTGGGCAAGCACTACGCTCGCAAGATCTACAAAACCCCATACGTACCTATAGACAAGGAGG
TGAAGATAGATGGGTTTTATATGCGCATGACTCTCAAGGTGCTGACCTTGAGTGACGATCTGGGAGTGTACCGCAAC
GACAGGATGCACCGCGCAGTGAGCGCCAGCAGAAGGCGTGAGCTGAGCGACAGAGAACTTATGCACAGCTTGCAAAG
AGCTCTGACTGGGGCTGGAACCGAGGGGGAAAACTACTTTGACATGGGAGCGGACTTGCAGTGGCAGCCCAGTCGCA
GGGCCCTGGACGCAGCAGGGTATGAGCTTCCTTACATAGAAGAGGTGGATGAAGGCCAGGATGAGGAGGGCGAGTAT
CTGGAAGACTGATGGCGCGACCATCCATATTTTTGCTAGATGGAACAGCAGGCACCGGACCCCGCAAAACGGGCGGC
GCTACAGAGCCAGCCGTCCGGCATTAACTCCTCGGACGATTGGAGCCAGGCCATGCAACGCATCATGGCGCTGACGA
CCCGCAACCCCGAAGCCTTTAGACAGCAACCCCAGGCCAACCGCCTTTCTGCCATCCTGGAGGCCGTAGTGCCCTCC
CGCTCCAACCCCACACACGAGAAGGTCCTGGCCATCGTGAACGCGCTGGTGGAGAACAAAGCCATACGTCCCGATGA
GGCTGGGCTGGTATACAATGCCCTATTGGAGCGCGTAGCCCGTTACAACAGCAGCAACGTGCAGACCAACCTGGACC
GGATGGTGACCGATGTGCGCGAGGCCGTGTCTCAGCGCGAGCGGTTCCAGCGAGACGCCAATTTAGGGTCGCTGGTG
GCTTTGAACGCCTTCCTCAGCACTCAGCCTGCCAACGTGCCTCGCGGTCAGCAAGACTACACAAACTTTCTAAGTGC
ATTGAGACTCATGGTGGCCGAAGTCCCTCAAAGCGAAGTGTACCAGTCCGGGCCAGACTACTTTTTCCAGACCAGCA
GACAGGGCTTGCAGACAGTGAACCTGAGCCAGGCTTTTAAGAACCTGAATGGTCTGTGGGGAGTGCGCGCCCCAGTG
GGAGATCGGGCGACCGTGTCTAGCTTGCTGACCCCCAACTCCCGCCTACTACTGCTCTTGGTAGCCCCATTCACTGA
CAGCGGTAGCATCGACCGTAATTCGTACTTGGGCTATCTGTTGAACCTGTATCGCGAGGCCATAGGGCAAACTCAGG
TAGATGAGCAAACCTATCAAGAAATTACCCAAGTGAGCCGCGCTCTGGGTCGGGAGGACACTGGCAGCTTGGAAGCC
ACCTTAAACTTCTTGCTGACCAACCGGTCGCAGAAGATCCCTCCTCAGTATTCGCTTACCGCGGAGGAGGAACGGAT
CCTGAGATACGTGCAGCAGAGCGTGGGACTGTTCCTAATGCAGGAGGGGGCGACTCCTACTGCTGCGCTCGATATGA
CAGCCCGAAACATGGAGCCCAGCATGTATGCCAGTAACCGGCCTTTTATCAATAAACTGCTAGACTACTTACACAGG
GCGGCTGCTATGAACTCTGATTATTTCACCAATGCTATCCTGAACCCCCATTGGCTGCCCCCACCTGGGTTCTATAC
GGGCGAGTATGACATGCCCGACCCCAATGACGGGTTTTTATGGGACGATGTGGACAGTAGTGTTTTCTCCCCGCCTC
CTGGTTATAACACTTGGAAGAAGGAAGGTGGCGATAGAAGGCACTCTTCCGTGTCACTGTCCGGAGCAACGGGTGCT
GCAGCGGCTCCCGAGGCCGCAAGTCCTTTCCCTAGTTTGCCATTTTCGCTAAACAGTGTACGCAGCAGTGAGCTGGG
AAGAATAACCCGTCCTCGCTTGATCGGCGAGGAGGAGTATTTGAACGACTCCCTGTTGAGACCCGAGAGGGAGAAGA
ATTTCCCCAACAACGGGATAGAAAGCTTGGTTGACAAAATGAACCGCTGGAAGACGTACGCGCACGATCACAGGGAC
GATCCCCGGGCGCTGGGGGATAGCCGGGGCATCGCTACCCGTAAACGCCAGTGGCACGACAGGCAGCGGGGCCTGGT
GTGGGCCGATGATGATTCCGCCGACGACAGCAGCGTGTTGGACTTGGGTGGGAGTGGTGGTAACCCGTTCGCTCACC
TGCGCCCCCGCGTCGGGCGCCTGATGTAAGAAACCGAAAATAAATACTCACCAAGGCCATGGCGACCAGCGTGCGTT
CGTTTCTTCTCTGTTATAGCTAGTATGATGAGGCGAACCGTGCTAGGCGGAGCGGTGGTGTATCCGGAGGGTCCTCC
TCCTTCGTACGAGAGCGTGATGCAGCAGGCGGCGGCGGCGGCGATGCAGCCACCACTGGAGGCTCCCTTTGTACCCC
CTCGGTACCTGGCACCTACGGAGGGGAGAAACAGCATTCGTTACTCGGAGCTGGCACCATTGTATGATACCACCCGG
TTGTATTTGGTGGACAACAAGTCCGCGGACATCGCCTCACTGAACTATCAGAACGACCACAGCAACTTCCTCACCAC
GGTGGTGCAAAACAATGACTTTACCCCCACGGAGGCCAGCACCCAGACCATCAACTTTGACGAGCGGTCGCGATGGG
GCGGTCAGCTGAAGACTATCATGCACACCAACATGCCCAACGTGAACGAGTACATGTTTAGCAACAAGTTCAAAGCT
CGGGTGATGGTGTCTAGAAAGGCTCCTGAAGGTGTCACAGTAGATGACAATTATGATCACAAGCAGGATATTTTGGA
ATATGAGTGGTTTGAGTTTACTCTACCGGAAGGGAACTTCTCAGCCACAATGACCATTGACCTAATGAACAATGCCA
TCATTGATAATTACCTTGAAGTGGGCAGACAGAATGGAGTGTTGGAGAGTGACATTGGTGTTAAATTTGACACCAGG
AACTTTAGACTGGGTTGGGATCCGGAAACTAAGTTGATTATGCCTGGGGTTTACACCTATGAGGCATTCCATCCTGA
CATTGTATTGTTGCCTGGTTGCGGAGTTGACTTTACTGAAAGTCGCCTTAGTAACTTGCTTGGTATCAGGAAAAGAC
ACCCATTCCAGGAGGGTTTTAAGATCTTGTATGAGGATCTTGAAGGGGGTAATATCCCGGCCCTGTTGGATGTAGAA
GCCTATAAGAACAGTAAGAAAGAACGAGAAGCCAAAACAGAAGCCGCTAAAGCTGCTGCTATTGCTAAAGCCAACAT
AGTTGTCAGCGACCCTGTAAGGGTGGCTAATGCCGAAGAAGTCAGAGGAGACAACTATACAGCTTCATCTGTTGCAA
CTGAAGAATCGCTATTGGCTGCTGTGGCCGAAACTACAGAGACCAAACTCACTATTAAACCTGTAGAAAAAGACAGC
AAGAGTAGAAGTTACAATGTCTTGGAAGATAAAGTGAATACAGCCTACCGCAGCTGGTACCTGTCCTACAACTATGG
TGACCCTGAAAAAGGAGTCCGTTCCTGGACACTGCTCACCACCTCGGATGTCACCTGTGGAGCAGAGCAGGTGTACT
GGTCGCTCCCAGACATGATGCAGGACCCTGTCACATTCCGTTCCACGAGACAAGTCAGCAACTATCCAGTGGTAGGT
GCAGAGCTCATGCCGGTCTTCTCAAAGAGTTTCTACAACGAGCAAGCCGTGTACTCCCAGCAGCTTCGCCAGTCCAC
CTCGCTCACGCACGTCTTCAACCGCTTCCCTGAGAACCAGATCCTCATCCGCCCGCCAGCGCCCACCATTACCACCG
TCAGTGAAAACGTTCCTGCTCTCACAGATCACGGGACCCTGCCGTTGCGCAGCAGTATCCGGGGAGTCCAGCGCGTG
ACCGTTACTGACGCCAGACGCCGCACCTGCCCCTACGTCTACAAGGCCCTGGGCATAGTCGCGCCGCGCGTCCTTTC
AAGCCGCACTTTCTAAAAAAAAATGTCCATTCTTATCTCACCTAGTAATAACACCGGTTGGGGCCTGCGCGCGCCAA
GCAAGATGTACGGAGGTGCTCGCAAACGCTCTACACAGCACCCTGTGCGCGTGCGCGGGCACTTCCGCGCTCCATGG
GGCGCCCTCAAGGGTCGTACCCGCACTAGAACCACCGTCGATGATGTGATCGACCAGGTGGTGGCCGATGCTCGTAA
TTATACTCCTACTGCACCTACATCTACTGTGGATGCAGTTATTGACAGCGTAGTGGCTGACGCCCGCGCCTATGCTC
GCCGGAAGAGCAGGCGGAGACGCATCGCCAGGCGCCACCGGGCTACTCCCGCTATGCGAGCGGCAAGAGCTCTGCTG
AGGAGGGCCAAACGCGTGGGGCGAAGAGCTATGCTTAGAGCGGCCAGACGCGCGGCTTCAGGTGCCAGTGCCGGCAG
GTCCCGCAGGCGCGCAGCCACGGCGGCAGCAGCGGCCATTGCCAACATGGCCCAACCGCGAAGAGGCAATGTGTACT
GGGTGCGCGACGCCACCACCGGCCAGCGCGTGCCCGTGCGCACCCGCCCCCCTCGCTCTTAGAAGATACTGAGCAGT
CTCCGATGTTGTGTCCCAGCGAGGATGTCCAAGCGCAAATACAAGGAAGAGATGCTCCAGGTCATCGCGCCTGAAAT
CTACGGTCCGCCGGTGAAGGATGAAAAAAAGCCCCGCAAAATCAAGCGGGTCAAAAAGGACAAAAAGGAAGAAGATG
GCAATGATGGTCTGGCGGAGTTTGTACGCGAGTTCGCCCCAAGGCGGCGAGTGCAGTGGCGTGGACGCAAAGTGCAG
CCTGTGCTGAGACCTGGAACCACGGTGGTCTTTACGCCCGGCGAGCGCTCCAGCACTGCTTTTAAGCGGTCCTATGA
TGAGGTGTATGGGGATGATGATATTCTGGAGCAGGCGGCCGACCGCCTGGGCGAGTTTGCTTATGGCAAGCGCTCCC
GCTCGAGCCCCAAGGAGGAGGCGGTGTCCATTCCCTTGGACAATGGGAATCCCACCCCTAGTCTCAAGCCAGTCACC
CTGCAGCAAGTGCTGCCCGTGCCTCCACGCAGAGGCAACAAGCGAGAGGGTGAGGATCTGTATCCCACTATGCAATT
GATGGTGCCCAAGCGCCAGCGGCTGGAGGACGTGCTGGAGAAAATGAAAGTGGATCCCGATATACAACCTGAGGTCA
AAGTGAGACCCATCAAGCAGGTGGCGCCAGGTTTGGGAGTACAAACCGTAGACATCAAGATTCCCACCGAGTCAATG
GAAGTCCAAACCGAACCTGCAAAGCCCACAACCACCTCCATTGAGGTGCAAACGGATCCCTGGATGACCGCACCCGT
TACAACTCCAGCTGCTGTCAACACCACTCGAAGATCCCGGCGAAAGTACGGTCCAGCAAGTTTGCTGATGCCAAATT
ATGCTCTGCACCCATCCATTATTCCAACTCCGGGTTACCGAGGCACTCGCTACTACCGCAGCAGGAGCAGCACTTCC
CGCCGTCGCCGCAAAACACCTGCAAGTCGTAGTCATCGTCGTCGCCGCCGCCCCACCAGCAATCTGACTCCCGCTGC
TCTGGTGCGGAGAGTGTATCGCGATGGCCGCGCGGATCCCCTGACGTTACCGCGCGTACGCTACCATCCAAGCATCA
CAACTTAACAACTGTTGCCGCTGCCTCCTTGCAGATATGGCCCTCACTTGCCGCCTTCGTGTCCCCATTACTGGCTA
CCGAGGAAGAAACTCGCGCCGTAGAAGAGGGATGTTGGGGCGCGGAATGCGACGCCACAGGCGGCGGCGCGCTATCA
GCAAGAGGCTGGGGGGTGGCTTTCTGCCTGCTCTGATCCCCATCATAGCCGCGGCGATCGGGGCGATACCAGGCATA
GCTTCCGTGGCGGTTCAGGCCTCGCAGCGCCACTGACATTGGAAAAACTTATAAATAAAACAGAATGGACTCTGATG
CTCCTGGTCCTGTGACTATGTTTTTGTAGAGATGGAAGACATCAATTTTTCATCCCTGGCTCCGCGACACGGCACGA
GGCCGTACATGGGCACCTGGAGCGACATCGGCACCAGCCAACTGAACGGGGGCGCCTTCAATTGGAGCAGTATCTGG
AGCGGGCTTAAAAATTTTGGCTCTACCATAAAAACCTATGGGAACAAAGCTTGGAACAGCAGCACAGGGCAGGCATT
GAAAAATAAGCTTAAAGAGCAAAACTTCCAACAGAAGGTGGTTGATGGAATCGCCTCTGGTATCAATGGGGTGGTGG
ATCTGGCCAACCAGGCCGTGCAGAAACAGATAAACAGCCGCCTTGACCCGCCGCCGTCAGCCCCTGGTGAAATGGAA
GTGGAGGAAGATCTCCCTCCCCTTGAAAAGCGGGGCGACAAGCGTCCGCGCCCCGATCTGGAGGAGACACTAGTCAC
ACGCTCAGACGACCCGCCCTCCTACGAGGAGGCAGTGAAGCTTGGAATGCCCACCACCAGACCTGTAGCCCCCATGG
CTACCGGGGTAATGAAACCTTCTCAGTCACACCGACCCGCTACCTTGGACTTGCCTCCCCCTGCTGTTGCAGCGCCT
GCTCGCAAGCCTGTCGCTACCCCGAAGCCCACCACCGTACAGCCCGTCGCCGTAGCCAGACCGCGTCCTGGGGGCAC
TCCACGTCCGAATGCAAACTGGCAGAGTACTCTGAACAGCATCGTGGGTCTGGGCGTGCAAAGTGTAAAGCGCCGTC
GCTGCTTTTAAATTAAATATGGAGTAGCGCTTAACTTGCCTGTCTGTGTGTATGTGTCATCATCACGCCGCCGCCGC
AGCAACAGCAGAGGAGCAAGGAAGAGGTCGCGCGCCGAGGCTGAGTTGATTTCAAGATGGCCACCCCATCGATGCTG
CCCCAGTGGGCATACATGCACATCGCCGGACAGGATGCTTCGGAGTACCTGAGTCCGGGTCTGGTGCAGTTCGCCCG
CGCCACAGACACCTACTTCAATCTGGGGAACAAGTTTAGGAACCCCACCGTGGCGCCCACCCATGATGTGACCACCG
ACCGCAGTCAGCGGCTGATGCTGCGCTTTGTGCCCGTTGACCGGGAAGACAATACCTACGCATACAAAGTTCGATAC
ACCTTGGCTGTGGGCGACAACAGAGTGCTGGATATGGCCAGCACTTTCTTTGACATTCGGGGTGTGTTGGATAGAGG
TCCTAGCTTCAAGCCATATTCTGGCACTGCTTACAACTCATTGGCCCCTAAGGGCGCTCCCAATACATCTCAGTGGC
TTAATAAGGGAGATGAAGAGGATGGGGAAGACGACCAACAAGCTACATACACTTTTGGCAATGCGCCAGTAAAAGCC
GAAGCTGAAATTACAAAAGAAGGACTGCCAATAGGTTTGGAAGTTCCATCTGAAGGTGGCCCTAAACCCATTTATGC
TGATAAACTGTATCAGCCAGAACCTCAGGTGGGAGAGGAATCTTGGACTGATACGGATGGCACAGATGAAAAATATG
GAGGCAGAGCACTTAAACCTGAAACTAAAATGAAACCCTGCTACGGGTCTTTCGCTAAACCTACTAATGTTAAAGGC
GGGCAGGCAAAAGTGAAGAAAGAAGAAGAAGGCAAGGTTGAATATGACATTGACATGAACTTTTTCGACCTAAGATC
ACAAATGACTGGCCTCAAGCCTAAAATTGTAATGTATGCAGAAAATGTGGATCTAGAAACTCCTGACACTCATGTGG
TGTACAAACCTGGAGCTTCAGATGCTAGCTCTCATGCAAACCTTGGTCAACAGTCCATGCCCAATAGACCTAACTAT
ATTGGCTTCAGGGACAACTTCATCGGACTCATGTACTACAACAGTACTGGCAACATGGGAGTGCTGGCTGGACAAGC
GTCTCAGCTAAATGCAGTGGTTGACTTGCAAGACAGAAACACAGAATTGTCATATCAACTCTTGCTTGATTCTCTGG
GGGACAGAACCAGATATTTCAGTATGTGGAATCAAGCAGTGGATAGCTATGACCCAGATGTGCGTGTTATTGAGAAC
CATGGTGTGGAAGATGAACTTCCTAACTATTGTTTTCCATTGGATGGTGTAGGTCCGCGAATAGACAGTTACAAGGG
AATTGAGACAAATGGTGATGAAACCACTACTTGGAAAGATTTAGAGCCAAAGGGCATAAGTGAAATTGCTAAGGGAA
ATCCGTTTGCCATGGAAATTAACCTCCAAGCTAATCTCTGGAGAAGTTTTCTTTATTCCAATGTGGCTCTGTATCTC
CCAGACTCCTACAAATACACCCCAGCCAATGTCACTCTTCCAACTAACACCAACACTTATGACTACATGAATGGGCG
GGTGGTTCCCCCATCCCTGGTGGATACCTACGTAAACATTGGCGCCAGATGGTCTTTGGATGCCATGGACAATGTCA
ACCCCTTTAACCATCACCGCAACGCTGGCCTGCGATACCGGTCCATGCTTTTGGGCAATGGTCGTTACGTGCCTTTC
CACATTCAAGTGCCTCAGAAATTCTTTGCTGTGAAGAACCTGCTGCTTCTACCCGGTTCTTACACCTACGAGTGGAA
CTTCAGAAAGGATGTGAACATGGTCCTGCAGAGTTCCCTTGGTAATGATCTCCGGGTCGATGGTGCCAGCATAAGTT
TTACCAGCATCAATCTCTATGCCACCTTCTTCCCCATGGCCCACAACACTGCCTCCACCCTTGAAGCCATGCTGCGC
AATGACACCAATGATCAATCATTCAATGACTACCTTTCTGCTGCCAACATGCTCTACCCCATCCCGGCCAACGCTAC
CAACGTTCCCATCTCCATTCCCTCTCGCAACTGGGCCGCCTTCAGAGGCTGGTCCTTCACCAGACTCAAAACCAAGG
AGACTCCCTCTTTGGGATCAGGGTTCGATCCCTACTTTGTTTACTCTGGTTCTATACCCTACCTGGATGGTACCTTC
TACCTTAACCACACTTTCAAGAAAGTCTCCATCATGTTTGACTCTTCAGTGAGCTGGCCTGGTAATGACAGATTGCT
AAGTCCAAATGAGTTCGAAATCAAGCGCACAGTTGATGGGGAAGGCTACAATGTGGCCCAATGTAACATGACCAAAG
ACTGGTTCCTGGTCCAGATGCTTGCCAACTACAACATTGGATACCAGGGCTTCTACGTTCCTGAGGGTTACAAGGAT
CGCATGTACTCCTTCTTCAGAAACTTCCAGCCCATGAGTAGACAGGTGGTTGATGAGATTAACTACAAAGACTATAA
AGCTGTCGCCGTACCCTACCAGCATAATAACTCTGGCTTTGTGGGTTACATGGCTCCTACCATGCGTCAGGGTCAAG
CGTACCCTGCTAACTACCCATACCCCCTAATTGGAACCACTGCAGTAACCAGTGTCACCCAGAAAAAATTCCTGTGC
GACAGGACCATGTGGCGCATCCCATTCTCTAGCAACTTCATGTCCATGGGTGCCCTTACAGACCTGGGACAGAACTT
GCTGTATGCCAACTCGGCCCATGCGCTGGACATGACTTTTGAGGTGGATCCCATGGATGAGCCCACCCTGCTTTATC
TTCTTTTCGAAGTCTTCGACGTGGTCAGAGTGCACCAGCCACACCGCGGCGTCATCGAGGCCGTCTACCTGCGCACA
CCGTTCTCGGCCGGCAACGCCACCACATAAGAAGCCTCTTGCTTCTTGCAAGCAGCAGCTGCAGCCATGTCATGCGG
GTCCGGAAACGGCTCCAGCGAGCAAGAGCTCAAAGCCATCGTCCGAGACCTGGGCTGCGGACCCTATTTCCTGGGAA
CCTTTGACAAGCGTTTCCCGGGGTTCATGGCCCCCGACAAGCTCGCCTGCGCCATAGTCAACACTGCCGGACGCGAG
ACGGGGGGAGAGCACTGGCTGGCTTTTGGTTGGAACCCGCGCTCCAACACCTGCTACCTTTTTGATCCTTTTGGGTT
CTCGGATGAGCGACTCAAACAGATTTACCAGTTTGAGTACGAGGGGCTCCTGCGCCGCAGTGCCCTTGCTACCAAAG
ACCGCTGCATCACCCTGGAAAAGTCCACCCAGAGCGTGCAGGGCCCGCGCTCAGCCGCCTGTGGACTTTTTTGCTGT
ATGTTCCTTCATGCCTTTGTGCACTGGCCCGACCGCCCCATGAACGGAAACCCCACCATGAAGTTGCTGACTGGGGT
GTCAAACAGCATGCTCCAATCACCCCAAGTCCAGCCCACCCTGCGCCGCAACCAGGAGGCGCTATATCGCTTCCTAA
ACACCCACTCATCTTACTTTCGTTCTCACCGCGCACGCATTGAAAGGGCCACCGCGTTTGACCGTATGGATATGCAA
TAAGTCATGTAAAACCGTGTTCAATAAAAAGCATTTTATTTTTACATGCACTAAGGCTCTGGTTTTTTGCTCATTCG
TTTTCATCATTCACTCAGAAATCAAATGGGTTCTGGCGGGAGTCAGAGTGACCCGTGGGCAGGGAGACGTTGCGGAA
CTGTAACCTGTTCTGCCACTTGAACTCGGGGATCACCAGCTTGGGAACTGGAATTTCGGGAAAGGTGTCTTGCCACA
ACTTTCTGGTCAGTTGCAGGGCGCCAAGCAGGTCAGGAGCAGAGATCTTGAAATCACAGTTGGGGCCGGCATTCTGG
ACACGGGAGTTGCGGTACACTGGGTTGCAACACTGGAACACCATCAAGGCTGGGTGTCTCACGCTTGCCAGCACGGT
CGGGTCACTGATGGTAGTCACATCCAAGTCTTCAGCATTGGCCATTCCAAAGGGGGTCATCTTACAGGTCTGCCTGC
CCATCACGGGAGCGCAGCCTGGCTTGTGGTTGCAATCGCAATGAATGGGGATCAGCATCATCCTGGCTTGGTCGGGG
GTTATCCCTGGGTACACGGCCTTCATGAAGGCTTCGTACTGCTTGAAAGCTTCCTGAGCCTTACTTCCCTCGGTGTA
AAACATCCCACAGGACTTGCTGGAAAATTGGTTAGTAGCACAGTTGGCATCATTCACACAGCAGCGGGCATCGTTGT
TGGCCAACTGGACCACATTTCTGCCCCAGCGGTTCTGGGTGATCTTGGCTCTGTCTGGGTTCTCCTTCATAGCGCGC
TGCCCGTTTTCGCTCGCCACATCCATCTCGATAATGTGGTCCTTCTGGATCATGATAGTGCCATGCAGGCATTTCAC
CTTGCCTTCGTAATCGGTGCATCCATGAGCCCACAGAGCGCACCCGGTGCACTCCCAATTATTGTGGGCGATCTCAG
AATAAGAATGCACCAATCCCTGCATGAATCTTCCCATCATCGCTGTCAGGGTCTTCATGCTACTAAATGTCAGCGGG
ATGCCACGGTGCTCCTCGTTCACATACTGGTGGCAGATACGCTTGTACTGCTCGTGCTGCTCTGGCATCAGCTTGAA
AGAGGTTCTCAGGTCATTATCCAGCCTATACCTCTCCATTAGCACAGCCATCACTTCCATGCCCTTCTCCCAGGCAG
ATACCAGGGGCAAGCTCAAAGGATTCCTAACAGCAATAGAAGTAGCTCCTTTAGCTATAGGGTCATTCTTGTCGATC
TTCTCAACACTTCTCTTGCCATCCTTCTCAATGATGCGCACCGGGGGGTAGCTGAAGCCCACGGCCACCAACTGAGC
CTGTTCTCTTTCTTCTTCGCTGTCCTGGCTGATGTCTTGCAGAGGGACATGCTTGGTCTTCCTGGGCTTCTTCTTGG
GAGGGATCGGGGGAGGACTGTTGCTCCGTTCCGGAGACAGGGATGACCGCGAAGTTTCGCTTACCAGTACCACCTGG
CTCTCGATAGAAGAATCGGACCCCACGCGACGGTAGGTGTTCCTCTTCGGGGGCAGAGGTGGAGGCGACTGAGATGG
GCTGCGGTCCGGCCTTGGAGGCGGATGGCTGGCAGAGCCCATTCCGCGTTCGGGGGTGTGCTCCCGTTGGCGGTCGC
TTGACTGATTTCCTCCGCGGCTGGCCATTGTGTTCTCCTAGGCAGAGAAACAACAGACATGGAAACTCAGCCATCAC
TGCCAACATCGCTGCAAGCGCCATCACACCTCGCCCCCAGCAGCGACGAGGAGGAGAGCTTAACCACCCCACCACCC
AGTCCCGCTACCACCACCTCTACCCTCGATGATGAGGAGGAGGTCGACGCAGCCCAGGAGATGCAGGCGCAGGATAA
TGTGAAAGCGGAAGAGATTGAGGCAGATGTCGAGCAGGACCCGGGCTATGTGACACCGGCGGAGCACGAGGAGGAGC
TGAAACGTTTTCTAGACAGAGAGGATGACGACCGCCCAGAGCATCACCAGGAGGCTGGCCTCGGGGATCATGTTGCC
GACTACCTCTCCGGGCTTGGGAGGGAGGACGTGCTCCTCAAACATCTAGCAAGGCAGTCGATCATAGTTAAAGACGC
ACTACTCAACCTCACCGAAGTGCCTATCAGTGTGGAAGAGCTTAGCCGCGCCTACGAGCTGAACCTCTTTTCGCCTC
AGATACCCCCCAAGCGGCAGCGAAACGGCACCTGCGAGGCCAACCCTCGACTCAACTTCTATCCAGCTTTTACTGTC
CCCGAAGTGCTGGCCACCTACCACATCTTTTTTAAGAACCAAAAGATTCCAGTCTCCTGCCGCGCCAACCGCACCCG
CGCAGATGCCCTTCTCAACTTGGGTCCGGGAGCTCGCTTACCTGATATAGCTTCCTTGGAAGAGGTTCCAAAGATCT
TTGAGGGTCTGGGAAGTGATGAGACTCGGGCCGCAAATGCTCTGCAACAGGGAGAGAATGGTATGGATGAACATCAC
AGCGCTCTAGTGGAACTGGAGGGTGACAATGCCCGGCTTGCAGTGCTCAAGCGCAGTATCGTGGTCACCCATTTTGC
CTACCCCGCTGTTAACCTGCCGCCCAAAGTCATGAGCGCTGTCATGGACCATCTGCTCATCAAACGAGCAAGTCCAC
TTTCAGAAAACCAGAACATGCAGGATCCAGACGCCTCGGAGGAGGGCAAGCCGGTAGTCAGTGACGAGCAGCTATCT
CGCTGGCTGGGTACCAACTCCCCCCGAGATTTGGAAGAAAGACGCAAGCTTATGATGGCTGTAGTGCTAGTAACTGT
TGAGTTGGAGTGTCTGCGCCGCTTTTTTACCGACCCCGAGACCCTGCGCAAGCTAGAGGAGAACCTGCACTACACCT
TCAGACATGGCTTCGTGCGCCAGGCATGCAAGATCTCCAACGTGGAGCTCACCAACCTGGTTTCATACATGGGCATT
TTGCATGAGAACCGGCTAGGGCAGAGCGTTCTGCACACCACCCTGAAGGGGGAGGCCCGCCGCGACTACATCCGAGA
CTGTGTCTACCTCTACCTCTGCCATACCTGGCAGACTGGTATGGGTGTGTGGCAACAGTGTTTGGAAGAGCAGAACC
TTAAAGAGCTGGACAAGCTCTTGCAGAGATCCCTCAAAGCCCTGTGGACAGGTTTTGACGAGCGCACCGTCGCCTCG
GACCTGGCGGACATCATCTTCCCCGAGCGTCTTAGGGTTACTCTGCGAAACGGCCTGCCAGACTTCATGAGCCAGAG
CATGCTTAACAACTTTCGCTCTTTCATCCTGGAACGCTCCGGTATCCTGCCTGCCACCTGCTGTGCGCTGCCCTCCG
ACTTTGTGCCTCTCACCTACCGCGAGTGCCCACCGCCGCTATGGAGCCACTGCTACCTATTCCGCCTGGCCAACTAC
CTCTCCTACCACTCGGATGTGATAGAGGATGTGAGCGGAGACGGCCTGCTGGAATGCCACTGCCGATGCAATTTATG
CACACCCCACCGCTCCCTCGCCTGCAACCCCCAGTTGCTAAGCGAGACCCAGATCATCGGCACCTTCGAGTTGCAGG
GTCCCAACAGTGAAGGCGAGGGGTCTTCTCCGGGGCAGAGTCTGAAACTGACACCGGGGCTGTGGACCTCCGCCTAC
CTGCGCAAGTTTCATCCCGAGGACTATCATCCCTATGAGATCAGGTTCTATGAGGACCAGTCACATCCTCCCAAAGT
CGAGCTCTCAGCCTGCGTCATCACCCAGGGGGCAATTCTGGCCCAATTGCAAGCCATCCAAAAATCCCGCCAAGAAT
TTCTGCTGAAAAAGGGAAGCGGGGTCTACCTTGACCCCCAGACCGGTGAGGAGCTCAACACAAGGTTCCCCCAGGAT
GTCCCATCGCCGAGGAAGCAAGAAGCTGAAGGTGCAGCTGTCACCCCCAGAGGATATGGAGGAAGACTGGGACAGTC
AGGCAGAGGAGGAGATGGAAGATTGGGACAGCCAGGCAGAGGAGGTGGACAGCCTGGAGGAAGACAGTTTGGAGGAG
GAAGACGAGGAGGCAGAGGAGGTGGAAGAAGCAACCGCCGCCAAACAGTTGTCATCGGCGGCGGAGACAAGCAAGTC
CCCAGACAGCAGCACGGCTACCATCTCCGCTCCGGGTCGGGGGGCCCAGCGGCGGCCCAACAGTAGATGGGACGAGA
CCGGGCGATTTCCAAACCCGACCACCGCTTCCAAGACCGGTAAGAAGGAGCGACAGGGATACAAGTCCTGGCGTGGA
CATAAAAACGCTATCATCTCCTGCTTGCATGAATGCGGGGGCAACATATCCTTCACCCGGCGATACCTGCTTTTCCA
CCACGGTGTGAACTTCCCCCGCAATATCTTGCATTACTACCGTCACCTCCACAGCCCCTACTGCAGTCAGCAAGTCC
CGGCAACCCCGACAGAAAAAGACAGCAGCGACAACGGTGACCAGAAAAGCAGCAGTTAGAAAATCCACAACAAGTGC
AGCAGGAGGAGGACTGAGGATCACAGCGAACGAGCCAGCGCAGACCAGAGAGCTGAGGAACCGGATCTTTCCAACCC
TCTATGCCATCTTCCAGCAGAGTCGGGGGCAAGAGCAGGAATTGAAAGTAAAAAACCGATCTCTGCGCTCGCTCACC
AGAAGTTGTTTGTATCACAAGAGCGAAGACCAACTTCAGCGCACTCTCGAGGACGCCGAGGCTCTCTTCAACAAGTA
CTGCGCGCTGACTCTTAAAGAGTAGCCCTTGCCCGCGCTCATTCGAAAACGGCGGGAATCACGTCACCCTTGGCAGC
TGTCCTTTGCCCTCGTCATGAGTAAAGAGATTCCCACGCCTTACATGTGGAGCTATCAGCCCCAAATGGGGTTGGCA
GCAGGTGCTTCCCAGGACTACTCCACCCGCATGAATTGGCTTAGCGCCGGGCCCTCAATGATATCACGGGTTAATGA
TATACGAGCTTATCGAAACCAGTTACTCCTAGAACAGTCAGCTCTTACCACCACACCCCGCCAACACCTTAATCCCC
GAAATTGGCCCGCCGCCCTGGTGTACCAGGAAAATCCCGCTCCCACCACCGTACTACTTCCTCGAGACGCCCAGGCC
GAAGTTCAGATGACTAACGCAGGTGTACAGCTGGCGGGCGGTTCCGCCCTATGTCGTCACCGGCCTCAACAGAGTAT
AAAACGCCTGGTGATCAGAGGCCGAGGTATCCAGCTCAACGACGAGTCGGTTAGCTCTTCGCTTGGTCTGCGACCAG
ACGGAGTCTTCCAGATCGCCGGCTGTGGGAGATCTTCCTTCACTCCTCGTCAGGCTGTGCTGACTTTGGAGAGTTCG
TCCTCGCAGCCCCGCTCGGGCGGCATCGGAACTCTCCAGTTTGTGGAGGAGTTTACTCCCTCTGTCTACTTCAACCC
CTTCTCCGGCTCTCCTGGCCAGTACCCGGACGAGTTCATACCGAACTTCGACGCAATCAGCGAGTCAGTGGATGGCT
ATGATTGATGTCTAATGGTGGCGCGGCTGAGCTAGCTCGACTGCGACACCTAGACCACTGCCGCCGCTTTCGCTGTT
TCGCCCGGGAACTCACCGAGTTCATCTACTTCGAACTCTCCGAGGAGCACCCTCAGGGTCCGGCCCACGGAGTGCGG
ATTACCATCGAAGGGGGAATAGACTCTCGCCTGCATCGCATCTTCTCCCAGCGGCCCGTGCTGATTGAGCGCGACCA
GGGAAATACAACCATCTCCATCTACTGCATCTGTAACCACCCCGGATTGCATGAAAGCCTTTGCTGTCTTGTTTGTG
CTGAGTTTAATAAAAACTGAGTTAAGACCTTCCTACGGACTACCGCTTCTTCAATCAGGACTTTACAACACCAACCA
GATCTTCCAGAAGACCCAGACCCTTCCTCCTCTGATCCAGGACTCTAACTCTACCTTACCAGCACCCTCCACTACTA
ACCTTCCCGAAACTAACAAGCTTGGATCTCATCTGCAACACCGCCTTTCACGAAGCCTTCTTTCTGCCAATACTACC
ACTCCCAAAACCGGAGGTGAGCTCCGCGGTCTTCCTACTGACGACCCCTGGGTGGTAGCGGGTTTTGTAACGTTAGG
ATTAGTTGCGGGTGGGCTTGTGCTAATCCTTTGCTACCTATACACACCTTGCTGTGCATATTTAGTCATATTGTGCT
GTTGGTTTAAGAAATGGGGGCCATACTAGTCGTGCTTGCTTTACTTTCGCTTTTGGGTCTGGGCTCTGCTAATCTCA
ATCCTCTTGATCACAATCCATGTCTAGACTTCGACCCAGAAAATTGCACACTTACTTTTGCACCCGACACAAGCCGT
CTCTGTGGAGTTCTTATTAAGTGCGGATGGGACTGCAGGTCCGTTGAAATTACACATAATAACAAAACATGGAACAA
TACCTTATCCACCACATGGGAGCCAGGAGTTCCCGAGTGGTATACTGTCTCTGTCCGAGGTCCTGACGGTTCCATTC
GCATTAGTAACAACACTTTCATTTTTTCTGAAATGTGCGATCTGGCCATGTTTATGAGCAAACAGTATGACCTATGG
CCTCCTAGCAAAGAGAACATTGTGGCATTTTCCATTGCTTATTGCTTGGTAACATGCATCATCACTGCTATCATTTG
TGTGTGCATACACTTGCTTATAGTTATTCGCCCTAGACAAAGCAATGAGGAAAAAGAGAAAATGCCTTAACCTTTTT
CCTCATACCTTTTCTTTACAGCATGGCTTCTGTTACAGCTCTAATTATTGCCAGCATTGTCACTGTCGCTCACGGGC
AAACAATTGTCCATATTACCTTAGGACATAATCACACTCTTGTAGGGCCCCCAATTACTTCAGAGGTTATTTGGACC
AAACTTGGAAGTGTTGATTATTTTGATATAATTTGCAACAAAACTGAACCAATATTTGTAATCTGTAACAGACAAAA
TCTCACGTTAATTAATGTTAGCAAAATTTATAACGGTTACTATTATGGTTATGATAGATCCAGTAGCCAATATAAAA
ATTACTTAGTTCGCATAACTCAGCCCAAATCAACAGTGCCAACTATGACAATAATTAAAATGGCTAATAAAGCATTA
GAAAATTTTACATTACCAACAACGCCCAATGAAAAAAACATTCCAAATTCAATGATTGCAATTATTGCGGCGGTGGC
ATTGGGAATGGCACTAATAATAATATGCATGTTCCTATATGCTTGTTGCTATAAAAAGTTTCAACATAAACAGGATC
CACTACTAAATTTTAACATTTAATTTTTTATACAGATGATTTCCACTACAATTTTTATCATTACTAGCCTTGCAGCT
GTAACTTATGGCCGTTCACACCTAACTGTACCTGTTGGCTCAACATGTACACTACAAGGACCCCAAGAAGGCTATGT
CACTTGGTGGAGAATATATGATAATGGAGGGTTCGCTAGACCATGTGATCAGCCTGGTACAAAATTTTCATGCAACG
GAAGAGACTTGACCATTATTAACATAACATTAAATGAGCAAGGCTTCTATTATGGAACCAACTATAAAAATAGTTTA
GATTACAACATTATTGTAGTGCCAGCCACCACTTCTGCTCCCCGCAAATCCACTTTCTCTAGCAGCAGTGCCAAAGC
AAGCACAATTCCTAAAACAGCTTCTGCTATGTTAAAGCTTCGAAAAATCGCTTTAAGTAATTCCACAGCAGCTCCCA
ATACAATTCCTAAATCAACAATTGGCATCATTACTGCCGTGGTAGTGGGATTAATGATTATATTTTTGTGCATAATG
TACTACGCCTGCTGCTATAGAAAACATGAACAAAAAGGTGATGCATTACTAAATTTTGATATTTAATTTTTTATAGA
ATTATGATATTGTTTCAATCCAATGCCACTAACACTATCAATGTGCAGACTACTTTAAAACATGACATGGAAAACCA
CACTACCTCCTATGCATACACAAATATTCAGCCTAAATACGCTATGCAACTAGAAATCACCATACTAATTGTAATTG
GAATTCTTATACTATCTGTTATTCTTTATTTTATATTCTGCCGTCAAATACCCAATGTTCATAGAAATTCTAAAAGA
CGTCCCATCTATTCTCCTATGATTAGTCGTCCCCATATGGCTCTGAATGAAATCTAAGATCTTTTTTTTTCTTTTAC
AGTATGGTGAACATCAATCATGATTCCTAGAAATTTCTTCTTCACCATACTCATCTGTGCTTTCAATGTCTGTGCTA
CTTTCACAGCAGTAGCCACTGCAAGCCCAGACTGTATAGGACCATTTGCTTCCTATGCACTTTTTGCCTTTGTTACT
TGCATCTGCGTGTGTAGCATAGTCTGCCTGGTTATTAATTTTTTCCAACTGGTAGACTGGATCTTTGTGCGAATTGC
CTACCTACGTCACCATCCCGAATACCGCAATCAAAATGTTGCGGCACTTCTTAGGCTTATTTAAAACCATGCAGGCT
ATGCTACCAGTTATTTTAATTCTGCTACTACCCTGCATTGCCCTACCTTCCACCGCCACTCGCGCTACACCTGAACA
ACTTAGAAAATGCAAATTTCAACAACCATGGTCATTTCTTGATTGCTACCATGAAAAATCTGATTTTCCCACATACT
GGATAGTGATTGTTGGAATAATTAACATACTTTCATGTACCTTTTTCTCAATCACAATATACCCCACATTTAATTTT
GGGTGGAATTCTCCCAATGCACTGGGTTACCCACAAGAGCTAGATGAACATATCCCACTACAACACATACAACAACC
ACTAGCATTGGTAGAGTATGAAAATGAGCCACAACCTTCACTGCCTCCTGCTATTAGTTACTTCAACCTAACCGGCG
GAGATGACTGAAATACTCACCACCTCCAATTCCGCCGAGGATCTGCTTGATATGGACGGCCGCGCCTCAGAACAGCG
ACTCGCCCAACTACGCATCCGCCAGCAGCAGGAACGCGTGACCAAAGAGCTCAGAGATGTCATCCAAATTCACCAAT
GCAAAAAAGGCATATTTTGTTTGGTAAAACAAGCCAAGATATCCTACGAGATCACCGCTACTGACCATCGCCTCTCT
TACGAACTTGGCCCCCAACGACAAAAATTTACATGCATGGTGGGAATCAACCCTATAGTTATCACCCAGCAAAGTGG
AGATACTAAGGGTTGCATTCACTGCTCTTGCGATTCCACCGAGTGCACCTACACCCTGCTGAAGACCCTATGCGGCC
TAAGAGACCTGCTACCCATGAATTAAAAATTAATAAAAAATTACTTACTTGAAATCAGCAATAAGGTCTCTGTTGAA
ATTTTTTCCCAGCAGCACCTCGCTTCCCTCTTCCCAACTCTGGTATTCTAAACCCCGTTCAGCGGCATACTTTCTCC
ATACTTTAAAGGGGATGTCAAATTTTAGCTCCTCTCCTGTACCCACGATCTTCATGTCTTTCTTCCCAGATGACCAA
GAGAGTCCGGCTCAGTGATTCCTTCAACCCTGTCTACCCCTATGAAGACGAAAGCACCTCCCAACACCCCTTTATAA
ACCCAGGGTTTATTTCCCCAAATGGCTTTACACAAAGCCCAGACGGAGTTCTTACTTTAAATTGTTTAACCCCACTA
ACAACCACAGGCGGGCCTTTACAGTTAAAAGTGGGAGGGGGACTTATAGTGGATGACACTGATGGGACCTTACAAGA
AAACATACGTGTTACAGCACCCATTACTAAAAATAATCATTCTGTAGAACTATCCATTGGAAATGGATTAGAAACAC
AAAACAATAAACTATGTGCCAAATTGGGAAATGGGTTAAAATTTAACAACGGTGACATTTGTATAAAGGATAGTATT
AACACCTTATGGACTGGAATAAAGCCTCCACCTAACTGTCAAATAGTGGAAAACACTGATACAAACGATGGCAAACT
TACTTTAGTATTAGTAAAAAACGGAGGACTTGTTAATGGCTACGTATCTCTAGTTGGTGTATCAGACACTGTGAACC
AAATGTTCACACAAAAGTCAGCAACCATACAATTAAGATTATATTTCGACTCTTCTGGAAATCTATTAACTGATGAA
TCAAACTTAAAAATTCCACTTAAAAATAAATCTTCTACAGCAACCAGTGAAGCTGCAACCAGCAGCAAAGCCTTTAT
GCCAAGTACTACAGCTTATCCCTTTAACACCACTACTAGGGATAGTGAAAACTATATTCATGGAATATGTTACTATA
TGACTAGTTATGATAGAAGTCTAGTTCCCTTAAACATTTCTATAATGCTAAACAGCCGTACGATTTCTTCCAATGTT
GCCTATGCCATACAATTTGAATGGAATCTAAATGCAAAAGAATCTCCAGAAAGCAACATAGCTACGCTGACCACATC
CCCCTTTTTCTTTTCTTATATTAGAGAAGACGACAACTAAAAAATAAAGTTTAAGTGTTTTTATTTAAAAATCACAA
AATTCGAGTAGTTATTTTGCCTCCCCCTTCCCATTTAACAGAATACACCAATCTCTCCCCACGCACAGCTTTAAACA
TTTGGATACCATTAGAGATAGACATAGTTTTAGTTTCCACATTCCAAACAGTTTCAGAGCGAGCCAATCIGGGGTCA
GTGATACATAAAAATGCATCGGGATAGTCTTTTAAAGCGCTTTCACAGTCCAACTGCTGCGGATGCGACTCCGGAGT
CTGGATCACAGTCATCTGGAAGAAGAACGATGGGAATCATAATCCGAAAACGGAATCGGGCGATTGTGTCTCATCAA
ACCCACAAGCAGCCGCTGTCTGCGTCGCTCCGTGCGACTGCTGTTTATGGGATCGGGGTCTGCAGTGTCCTGAAGCA
TGATTTTAATAGCCCTTAACATTAACTTTCTGGTGCGATGCGCGCAGCAACGCATTCTGATTTCACTGAGATTACTA
CAGTATGTACAGCACATTATCACAATATTGTTTAATAAACCATAATTAAAAGCGCTCCAGCCAAAACTCATATCTGA
TACAATCGCCCCTGCATGACCATCATACCAAATTTTAATATAAATTAAATGTCGTTCCCTCAAAAACACACTACCCA
CATACATGATCTCTTTTGGCATGTGCATATTAACAATCTGTCTGTACCATGGACAACGTTGGTTAATCATGCAACCC
AATATAACCTTCCGGAACCACACTGCCAACACCGCTCCCCCAGCCATGCATTGAAGTGAACCCTGCTGATTACAATG
ACAATGAAGAACCCAATTCTCTCGACCATGAATCACTTGAGACTGAAAAATATCTATAGTAGCACAACAAAGACATA
AATGCATGCATCTTCTCATAATTTTTAACTCATCTGGATTTAAAAACATATCCCAAGGAATGGGAAACTCTTGCAAA
ACAGTAAAGCTGGCAGAACAAGGAAGACCACGAACACAACTTACACTATGCATAGTCATAGTATCACAATCTGGCAA
CAGCGGGTGGTCTTCAGTCATAGAAGCTCGGGTTTCATTTTCCTCACATCGTGGTAACTGGGCTCTGGTGTAAGGGT
GATGTCTGGCGCATGATGTCGAGCGTGCGCGCAACCTTGTCATAATGGAGTTGTTTCCTGACATTCTCGTATTTTGT
ATAGCAAAACGCGGCCCTGGCACAACACACTCTTCTTCGTCTTCTATCCTGCCGCTTAGTGTGTTCCGTCTGATAAT
TCAAGTACAGCCACACTCTTAAGTTGGTCAAAAGAATGCTGGCTTCAGTTGTAATCAAAACTCCATCATATTTAATT
GTTCTAAGGAAATCATCCACGGTAGCATATGCAAATCCCAACCAAGCAATGCAACTGGATTGCGTTTCAAGCAGCAG
AGGAGAGGGAAGAGACGGAAGAATCATGTTAATTTTTATTCCAAACGATCTCGCAGTACTTCAAATTGTAGATCGCG
CAGATGGCATCTATCGCCCCCACTGTGTTGGTGAAAAAGCACAGCTAAATCAAAAGAAATGCGATTTTCAAGGTGCT
CAACGGTGGCTTCCAACAAAGCCTCCACGCGCACATCCAAAAACAAAAGAATACCAAAAGAAGGAGCATTTTCTAAC
TCCTCAAACATCATATTACATTCCTGCACCATTCCCAGATAATTTTCAGCTTTCCAGCCTTGAATTATTCGTGTCAG
TTCTTGTGGTAAATCCAAACCACACATTACAAACAGGTCCCGGAGGGCGCCCTCCACCACCATTCTTAAACACACCC
TCATAATGACAAAATATCTTGCTCCTGTGTCACCTGTAGCAAATTAAGAATGGCATCATCAATTGACATGCCCTTGG
CTCTAAGTTCTTCTCTAAGTTCTAGTTGTAAATACTCTCTCATATTATCACCAAACTGCTTAGCCAAAAGCCCCCCG
GGAACAATAGCAGGGGACGCTACAGTGCAGTACAAGCGCAGACCTCCCCAATTGGCTCCAGCAAAAACAAGATTAGA
ATAAGCATACTGGGAACCACCAGTAATATCATCAAAGTTGCTGGAAATATAATCAGGCAGAATTTCTTGTAAAAATT
GAATAAAAGAAAAATTTTCCAAAGAAACATTCAAAATCTCTGGGATGCAAATGCAATAGGTTACCGCGCTGCGCTCC
AACATTGTTAGTTTTGAATTAGTCTGCAAAATAAAAGAAACAAGCGTCATATCATAGTAGCCTGTCGAACAGGTGGA
TAAATCAGTCTTTCCATCACAAGACAAGCCACAGGGTCTCCAGCTCGACCCTCGTAAAACCTGTCATCGTGATTAAA
CAACAGCACCGAAAGTTCCTCGCGGTGGCCAGCATGAATAATTCTTGATGAAGCATATAATCCAGACATGTTAGCAT
CAGTTAAAGAGAAAAAACAGCCAACATAGCCTCTGGGTATAATTATGCTTAATCTTAAGTATAGCAAAGCCACCCCT
CGCGGATACAAAGTAAAAGGCACAGGAGAATAAAAAATATAATTATTTCTCTGCTGCTGTTCAGGCAACGTCGCCCC
CGGTCCATCTAAATACACATACAAAGCCTCATCAGCCATGGCTTACCAGACAAAGTACAGCGGGCGCACAAAGCACA
AGCTCTAAAGAAGCTCTAAAGACACTCTTCAACCTCTCCACAATATATACACAAGCCCTAAACTGACGTAATGGGAG
TAAAGTGTAAAAAATCCCGCCAAGCCCAACACACACCCCGAAACTGCGTCAGCAGGGAAAAGTACAGTTTCACTTCC
GCATTCCCAACAAGCGTAAGTTCCTCTTTCTCATGGTACGTCACATCCGATTAACTTGCAACGTCATTTTCCCACGG
TCGCACCGCCCCTTTTAGCCGTTCACCCCGCAGCCAATCACCACACAGCGCGCACTTTTTTAAATTACCTCATTTAC
ATATTGGCACCATTCCATCTATAAGGTATATTATATTGATTG

GenBank Accession No. AAW33547 (SEQ ID NO: 238)

MTKRVRLSDSFNPVYPYEDESTSQHPFINPGFISPNGFTQSPDGVLTLNCLTPLTTTGGPLQLKVGGGLIVDDTDGT
LQENIRVTAPITKNNHSVELSIGNGLETONNKLCAKLGNGLKFNNGDICIKDSINTLWTGIKPPPNCQIVENTDIND
GKLTLVLVKNGGLVNGYVSLVGVSDTVNQMFTQKSATIQLRLYFDSSGNLLTDESNLKIPLKNKSSTATSEAATSSK
AFMPSTTAYPFNTTTRDSENYIHGICYYMTSYDRSLVPLNISIMLNSRTISSNVAYAIQFEWNLNAKESPESNIATL
TTSPFFFSYIREDDN

GenBank Accession No. AAW33525 (SEQ ID NO: 239)

MRRTVLGGAVVYPEGPPPSYESVMQQAAAAAMQPPLEAPFVPPRYLAPTEGRNSIRYSELAPLYDTTRLYLVDNKSA
DIASLNYQNDHSNFLTTVVONNDFTPTEASTQTINFDERSRWGGQLKTIMHTNMPNVNEYMFSNKFKARVMVSRKAP
EGVTVDDNYDHKQDILEYEWFEFTLPEGNFSATMTIDLMNNAIIDNYLEVGRONGVLESDIGVKFDTRNFRLGWDPE
TKLIMPGVYTYEAFHPDIVLLPGCGVDFTESRLSNLLGIRKRHPFQEGFKILYEDLEGGNIPALLDVEAYKNSKKER
EAKTEAAKAAAIAKANIVVSDPVRVANAEEVRGDNYTASSVATEESLLAAVAETTETKLTIKPVEKDSKSRSYNVLE
DKVNTAYRSWYLSYNYGDPEKGVRSWTLLTTSDVTCGAEQVYWSLPDMMQDPVTFRSTRQVSNYPVVGAELMPVFSK
SFYNEQAVYSQQLRQSTSLTHVFNRFPENQILIRPPAPTITTVSENVPALTDHGTLPLRSSIRGVQRVTVTDARRRT
CPYVYKALGIVAPRVLSSRTF

GenBank Accession No. AAW33530 (SEQ ID NO: 240)

MATPSMLPQWAYMHIAGQDASEYLSPGLVQFARATDTYFNLGNKFRNPTVAPTHDVTTDRSQRLMLRFVPVDREDNT
YAYKVRYTLAVGDNRVLDMASTFFDIRGVLDRGPSFKPYSGTAYNSLAPKGAPNTSQWLNKGDEEDGEDDQQATYTF
GNAPVKAEAEITKEGLPIGLEVPSEGGPKPIYADKLYQPEPQVGEESWTDTDGTDEKYGGRALKPETKMKPCYGSFA
KPTNVKGGQAKVKKEEEGKVEYDIDMNFFDLRSQMTGLKPKIVMYAENVDLETPDTHVVYKPGASDASSHANLGQQS
MPNRPNYIGFRDNFIGLMYYNSTGNMGVLAGOASQLNAVVDLQDRNTELSYQLLLDSLGDRTRYFSMWNQAVDSYDP
DVRVIENHGVEDELPNYCFPLDGVGPRIDSYKGIETNGDETTTWKDLEPKGISEIAKGNPFAMEINLQANLWRSFLY
SNVALYLPDSYKYTPANVTLPTNTNTYDYMNGRVVPPSLVDTYVNIGARWSLDAMDNVNPFNHHRNAGLRYRSMLLG
NGRYVPFHIQVPQKFFAVKNLLLLPGSYTYEWNFRKDVNMVLQSSLGNDLRVDGASISFTSINLYATFFPMAHNTAS
TLEAMLRNDTNDQSFNDYLSAANMLYPIPANATNVPISIPSRNWAAFRGWSFTRLKTKETPSLGSGFDPYFVYSGSI
PYLDGTFYLNHTFKKVSIMFDSSVSWPGNDRLLSPNEFEIKRTVDGEGYNVAQCNMTKDWFLVQMLANYNIGYQGFY
VPEGYKDRMYSFFRNFQPMSRQVVDEINYKDYKAVAVPYQHNNSGFVGYMAPTMRQGQAYPANYPYPLIGTTAVTSV
TQKKFLCDRTMWRIPFSSNFMSMGALTDLGQNLLYANSAHALDMTFEVDPMDEPTLLYLLFEVFDVVRVHQPHRGVI
EAVYLRTPFSAGNATT

GenBank Accession No. AC_000008 (SEQ ID NO: 208)

CATCATCAATAATATACCTTATTTTGGATTGAAGCCAATATGATAATGAGGGGGTGGAGTTTGTGACGTGGCGCGGG
GCGTGGGAACGGGGCGGGTGACGTAGTAGTGTGGCGGAAGTGTGATGTTGCAAGTGTGGCGGAACACATGTAAGCGA
CGGATGTGGCAAAAGTGACGTTTTTGGTGTGCGCCGGTGTACACAGGAAGTGACAATTTTCGCGCGGTTTTAGGCGG
ATGTTGTAGTAAATTTGGGCGTAACCGAGTAAGATTTGGCCATTTTCGCGGGAAAACTGAATAAGAGGAAGTGAAAT
CTGAATAATTTTGTGTTACTCATAGCGCGTAATATTTGTCTAGGGCCGCGGGGACTTTGACCGTTTACGTGGAGACT
CGCCCAGGTGTTTTTCTCAGGTGTTTTCCGCGTTCCGGGTCAAAGTTGGCGTTTTATTATTATAGTCAGCTGACGTG
TAGTGTATTTATACCCGGTGAGTTCCTCAAGAGGCCACTCTTGAGTGCCAGCGAGTAGAGTTTTCTCCTCCGAGCCG
CTCCGACACCGGGACTGAAAATGAGACATATTATCTGCCACGGAGGTGTTATTACCGAAGAAATGGCCGCCAGTCTT
TTGGACCAGCTGATCGAAGAGGTACTGGCTGATAATCTTCCACCTCCTAGCCATTTTGAACCACCTACCCTTCACGA
ACTGTATGATTTAGACGTGACGGCCCCCGAAGATCCCAACGAGGAGGCGGTTTCGCAGATTTTTCCCGACTCTGTAA
TGTTGGCGGTGCAGGAAGGGATTGACTTACTCACTTTTCCGCCGGCGCCCGGTTCTCCGGAGCCGCCTCACCTTTCC
CGGCAGCCCGAGCAGCCGGAGCAGAGAGCCTTGGGTCCGGTTTCTATGCCAAACCTTGTACCGGAGGTGATCGATCT
TACCTGCCACGAGGCTGGCTTTCCACCCAGTGACGACGAGGATGAAGAGGGTGAGGAGTTTGTGTTAGATTATGTGG
AGCACCCCGGGCACGGTTGCAGGTCTTGTCATTATCACCGGAGGAATACGGGGGACCCAGATATTATGTGTTCGCTT
TGCTATATGAGGACCTGTGGCATGTTTGTCTACAGTAAGTGAAAATTATGGGCAGTGGGTGATAGAGTGGTGGGTTT
GGTGTGGTAATTTTTTTTTTAATTTTTACAGTTTTGTGGTTTAAAGAATTTTGTATTGTGATTTTTTTAAAAGGTCC
TGTGTCTGAACCTGAGCCTGAGCCCGAGCCAGAACCGGAGCCTGCAAGACCTACCCGCCGTCCTAAAATGGCGCCTG
CTATCCTGAGACGCCCGACATCACCTGTGTCTAGAGAATGCAATAGTAGTACGGATAGCTGTGACTCCGGTCCTTCT
AACACACCTCCTGAGATACACCCGGTGGTCCCGCTGTGCCCCATTAAACCAGTTGCCGTGAGAGTTGGTGGGCGTCG
CCAGGCTGTGGAATGTATCGAGGACTTGCTTAACGAGCCTGGGCAACCTTTGGACTTGAGCTGTAAACGCCCCAGGC
CATAAGGTGTAAACCTGTGATTGCGTGTGTGGTTAACGCCTTTGTTTGCTGAATGAGTTGATGTAAGTTTAATAAAG
GGTGAGATAATGTTTAACTTGCATGGCGTGTTAAATGGGGCGGGGCTTAAAGGGTATATAATGCGCCGTGGGCTAAT
CTTGGTTACATCTGACCTCATGGAGGCTTGGGAGTGTTTGGAAGATTTTTCTGCTGTGCGTAACTTGCTGGAACAGA
GCTCTAACAGTACCTCTTGGTTTTGGAGGTTTCTGTGGGGCTCATCCCAGGCAAAGTTAGTCTGCAGAATTAAGGAG
GATTACAAGTGGGAATTTGAAGAGCTTTTGAAATCCTGTGGTGAGCTGTTTGATTCTTTGAATCTGGGTCACCAGGC
GCTTTTCCAAGAGAAGGTCATCAAGACTTTGGATTTTTCCACACCGGGGCGCGCTGCGGCTGCTGTTGCTTTTTTGA
GTTTTATAAAGGATAAATGGAGCGAAGAAACCCATCTGAGCGGGGGGTACCTGCTGGATTTTCTGGCCATGCATCTG
TGGAGAGCGGTTGTGAGACACAAGAATCGCCTGCTACTGTTGTCTTCCGTCCGCCCGGCGATAATACCGACGGAGGA
GCAGCAGCAGCAGCAGGAGGAAGCCAGGCGGCGGCGGCAGGAGCAGAGCCCATGGAACCCGAGAGCCGGCCTGGACC
CTCGGGAATGAATGTTGTACAGGTGGCTGAACTGTATCCAGAACTGAGACGCATTTTGACAATTACAGAGGATGGGC
AGGGGCTAAAGGGGGTAAAGAGGGAGCGGGGGGCTTGTGAGGCTACAGAGGAGGCTAGGAATCTAGCTTTTAGCTTA
ATGACCAGACACCGTCCTGAGTGTATTACTTTTCAACAGATCAAGGATAATTGCGCTAATGAGCTTGATCTGCTGGC
GCAGAAGTATTCCATAGAGCAGCTGACCACTTACTGGCTGCAGCCAGGGGATGATTTTGAGGAGGCTATTAGGGTAT
ATGCAAAGGTGGCACTTAGGCCAGATTGCAAGTACAAGATCAGCAAACTTGTAAATATCAGGAATTGTTGCTACATT
TCTGGGAACGGGGCCGAGGTGGAGATAGATACGGAGGATAGGGTGGCCTTTAGATGTAGCATGATAAATATGTGGCC
GGGGGTGCTTGGCATGGACGGGGTGGTTATTATGAATGTAAGGTTTACTGGCCCCAATTTTAGCGGTACGGTTTTCC
TGGCCAATACCAACCTTATCCTACACGGTGTAAGCTTCTATGGGTTTAACAATACCTGTGTGGAAGCCTGGACCGAT
GTAAGGGTTCGGGGCTGTGCCTTTTACTGCTGCTGGAAGGGGGTGGTGTGTCGCCCCAAAAGCAGGGCTTCAATTAA
GAAATGCCTCTTTGAAAGGTGTACCTTGGGTATCCTGTCTGAGGGTAACTCCAGGGTGCGCCACAATGTGGCCTCCG
ACTGTGGTTGCTTCATGCTAGTGAAAAGCGTGGCTGTGATTAAGCATAACATGGTATGTGGCAACTGCGAGGACAGG
GCCTCTCAGATGCTGACCTGCTCGGACGGCAACTGTCACCTGCTGAAGACCATTCACGTAGCCAGCCACTCTCGCAA
GGCCTGGCCAGTGTTTGAGCATAACATACTGACCCGCTGTTCCTTGCATTTGGGTAACAGGAGGGGGGTGTTCCTAC
CTTACCAATGCAATTTGAGTCACACTAAGATATTGCTTGAGCCCGAGAGCATGTCCAAGGTGAACCTGAACGGGGTG
TTTGACATGACCATGAAGATCTGGAAGGTGCTGAGGTACGATGAGACCCGCACCAGGTGCAGACCCTGCGAGTGTGG
CGGTAAACATATTAGGAACCAGCCTGTGATGCTGGATGTGACCGAGGAGCTGAGGCCCGATCACTTGGTGCTGGCCT
GCACCCGCGCTGAGTTTGGCTCTAGCGATGAAGATACAGATTGAGGTACTGAAATGTGTGGGCGTGGCTTAAGGGTG
GGAAAGAATATATAAGGTGGGGGTCTTATGTAGTTTTGTATCTGTTTTGCAGCAGCCGCCGCCGCCATGAGCACCAA
CTCGTTTGATGGAAGCATTGTGAGCTCATATTTGACAACGCGCATGCCCCCATGGGCCGGGGTGCGTCAGAATGTGA
TGGGCTCCAGCATTGATGGTCGCCCCGTCCTGCCCGCAAACTCTACTACCTTGACCTACGAGACCGTGTCTGGAACG
CCGTTGGAGACTGCAGCCTCCGCCGCCGCTTCAGCCGCTGCAGCCACCGCCCGCGGGATTGTGACTGACTTTGCTTT
CCTGAGCCCGCTTGCAAGCAGTGCAGCTTCCCGTTCATCCGCCCGCGATGACAAGTTGACGGCTCTTTTGGCACAAT
TGGATTCTTTGACCCGGGAACTTAATGTCGTTTCTCAGCAGCTGTTGGATCTGCGCCAGCAGGTTTCTGCCCTGAAG
GCTTCCTCCCCTCCCAATGCGGTTTAAAACATAAATAAAAAACCAGACTCTGTTTGGATTTGGATCAAGCAAGTGTC
TTGCTGTCTTTATTTAGGGGTTTTGCGCGCGCGGTAGGCCCGGGACCAGCGGTCTCGGTCGTTGAGGGTCCTGTGTA
TTTTTTCCAGGACGTGGTAAAGGTGACTCTGGATGTTCAGATACATGGGCATAAGCCCGTCTCTGGGGTGGAGGTAG
CACCACTGCAGAGCTTCATGCTGCGGGGTGGTGTTGTAGATGATCCAGTCGTAGCAGGAGCGCTGGGCGTGGTGCCT
AAAAATGTCTTTCAGTAGCAAGCTGATTGCCAGGGGCAGGCCCTTGGTGTAAGTGTTTACAAAGCGGTTAAGCTGGG
ATGGGTGCATACGTGGGGATATGAGATGCATCTTGGACTGTATTTTTAGGTTGGCTATGTTCCCAGCCATATCCCTC
CGGGGATTCATGTTGTGCAGAACCACCAGCACAGTGTATCCGGTGCACTTGGGAAATTTGTCATGTAGCTTAGAAGG
AAATGCGTGGAAGAACTTGGAGACGCCCTTGTGACCTCCAAGATTTTCCATGCATTCGTCCATAATGATGGCAATGG
GCCCACGGGCGGCGGCCTGGGCGAAGATATTTCTGGGATCACTAACGTCATAGTTGTGTTCCAGGATGAGATCGTCA
TAGGCCATTTTTACAAAGCGCGGGCGGAGGGTGCCAGACTGCGGTATAATGGTTCCATCCGGCCCAGGGGCGTAGTT
ACCCTCACAGATTTGCATTTCCCACGCTTTGAGTTCAGATGGGGGGATCATGTCTACCTGCGGGGCGATGAAGAAAA
CGGTTTCCGGGGTAGGGGAGATCAGCTGGGAAGAAAGCAGGTTCCTGAGCAGCTGCGACTTACCGCAGCCGGTGGGC
CCGTAAATCACACCTATTACCGGGTGCAACTGGTAGTTAAGAGAGCTGCAGCTGCCGTCATCCCTGAGCAGGGGGGC
CACTTCGTTAAGCATGTCCCTGACTCGCATGTTTTCCCTGACCAAATCCGCCAGAAGGCGCTCGCCGCCCAGCGATA
GCAGTTCTTGCAAGGAAGCAAAGTTTTTCAACGGTTTGAGACCGTCCGCCGTAGGCATGCTTTTGAGCGTTTGACCA
AGCAGTTCCAGGCGGTCCCACAGCTCGGTCACCTGCTCTACGGCATCTCGATCCAGCATATCTCCTCGTTTCGCGGG
TTGGGGCGGCTTTCGCTGTACGGCAGTAGTCGGTGCTCGTCCAGACGGGCCAGGGTCATGTCTTTCCACGGGCGCAG
GGTCCTCGTCAGCGTAGTCTGGGTCACGGTGAAGGGGTGCGCTCCGGGCTGCGCGCTGGCCAGGGTGCGCTTGAGGC
TGGTCCTGCTGGTGCTGAAGCGCTGCCGGTCTTCGCCCTGCGCGTCGGCCAGGTAGCATTTGACCATGGTGTCATAG
TCCAGCCCCTCCGCGGCGTGGCCCTTGGCGCGCAGCTTGCCCTTGGAGGAGGCGCCGCACGAGGGGCAGTGCAGACT
TTTGAGGGCGTAGAGCTTGGGCGCGAGAAATACCGATTCCGGGGAGTAGGCATCCGCGCCGCAGGCCCCGCAGACGG
TCTCGCATTCCACGAGCCAGGTGAGCTCTGGCCGTTCGGGGTCAAAAACCAGGTTTCCCCCATGCTTTTTGATGCGT
TTCTTACCTCTGGTTTCCATGAGCCGGTGTCCACGCTCGGTGACGAAAAGGCTGTCCGTGTCCCCGTATACAGACTT
GAGAGGCCTGTCCTCGAGCGGTGTTCCGCGGTCCTCCTCGTATAGAAACTCGGACCACTCTGAGACAAAGGCTCGCG
TCCAGGCCAGCACGAAGGAGGCTAAGTGGGAGGGGTAGCGGTCGTTGTCCACTAGGGGGTCCACTCGCTCCAGGGTG
TGAAGACACATGTCGCCCTCTTCGGCATCAAGGAAGGTGATTGGTTTGTAGGTGTAGGCCACGTGACCGGGTGTTCC
TGAAGGGGGGCTATAAAAGGGGGTGGGGGCGCGTTCGTCCTCACTCTCTTCCGCATCGCTGTCTGCGAGGGCCAGCT
GTTGGGGTGAGTACTCCCTCTGAAAAGCGGGCATGACTTCTGCGCTAAGATTGTCAGTTTCCAAAAACGAGGAGGAT
TTGATATTCACCTGGCCCGCGGTGATGCCTTTGAGGGTGGCCGCATCCATCTGGTCAGAAAAGACAATCTTTTTGTT
GTCAAGCTTGGTGGCAAACGACCCGTAGAGGGCGTTGGACAGCAACTTGGCGATGGAGCGCAGGGTTTGGTTTTTGT
CGCGATCGGCGCGCTCCTTGGCCGCGATGTTTAGCTGCACGTATTCGCGCGCAACGCACCGCCATTCGGGAAAGACG
GTGGTGCGCTCGTCGGGCACCAGGTGCACGCGCCAACCGCGGTTGTGCAGGGTGACAAGGTCAACGCTGGTGGCTAC
CTCTCCGCGTAGGCGCTCGTTGGTCCAGCAGAGGCGGCCGCCCTTGCGCGAGCAGAATGGCGGTAGGGGGTCTAGCT
GCGTCTCGTCCGGGGGGTCTGCGTCCACGGTAAAGACCCCGGGCAGCAGGCGCGCGTCGAAGTAGTCTATCTTGCAT
CCTTGCAAGTCTAGCGCCTGCTGCCATGCGCGGGCGGCAAGCGCGCGCTCGTATGGGTTGAGTGGGGGACCCCATGG
CATGGGGTGGGTGAGCGCGGAGGCGTACATGCCGCAAATGTCGTAAACGTAGAGGGGCTCTCTGAGTATTCCAAGAT
ATGTAGGGTAGCATCTTCCACCGCGGATGCTGGCGCGCACGTAATCGTATAGTTCGTGCGAGGGAGCGAGGAGGTCG
GGACCGAGGTTGCTACGGGCGGGCTGCTCTGCTCGGAAGACTATCTGCCTGAAGATGGCATGTGAGTTGGATGATAT
GGTTGGACGCTGGAAGACGTTGAAGCTGGCGTCTGTGAGACCTACCGCGTCACGCACGAAGGAGGCGTAGGAGTCGC
GCAGCTTGTTGACCAGCTCGGCGGTGACCTGCACGTCTAGGGCGCAGTAGTCCAGGGTTTCCTTGATGATGTCATAC
TTATCCTGTCCCTTTTTTTTCCACAGCTCGCGGTTGAGGACAAACTCTTCGCGGTCTTTCCAGTACTCTTGGATCGG
AAACCCGTCGGCCTCCGAACGGTAAGAGCCTAGCATGTAGAACTGGTTGACGGCCTGGTAGGCGCAGCATCCCTTTT
CTACGGGTAGCGCGTATGCCTGCGCGGCCTTCCGGAGCGAGGTGTGGGTGAGCGCAAAGGTGTCCCTGACCATGACT
TTGAGGTACTGGTATTTGAAGTCAGTGTCGTCGCATCCGCCCTGCTCCCAGAGCAAAAAGTCCGTGCGCTTTTTGGA
ACGCGGATTTGGCAGGGCGAAGGTGACATCGTTGAAGAGTATCTTTCCCGCGCGAGGCATAAAGTTGCGTGTGATGC
GGAAGGGTCCCGGCACCTCGGAACGGTTGTTAATTACCTGGGCGGCGAGCACGATCTCGTCAAAGCCGTTGATGTTG
TGGCCCACAATGTAAAGTTCCAAGAAGCGCGGGATGCCCTTGATGGAAGGCAATTTTTTAAGTTCCTCGTAGGTGAG
CTCTTCAGGGGAGCTGAGCCCGTGCTCTGAAAGGGCCCAGTCTGCAAGATGAGGGTTGGAAGCGACGAATGAGCTCC
ACAGGTCACGGGCCATTAGCATTTGCAGGTGGTCGCGAAAGGTCCTAAACTGGCGACCTATGGCCATTTTTTCTGGG
GTGATGCAGTAGAAGGTAAGCGGGTCTTGTTCCCAGCGGTCCCATCCAAGGTTCGCGGCTAGGTCTCGCGCGGCAGT
CACTAGAGGCTCATCTCCGCCGAACTTCATGACCAGCATGAAGGGCACGAGCTGCTTCCCAAAGGCCCCCATCCAAG
TATAGGTCTCTACATCGTAGGTGACAAAGAGACGCTCGGTGCGAGGATGCGAGCCGATCGGGAAGAACTGGATCTCC
CGCCACCAATTGGAGGAGTGGCTATTGATGTGGTGAAAGTAGAAGTCCCTGCGACGGGCCGAACACTCGTGCTGGCT
TTTGTAAAAACGTGCGCAGTACTGGCAGCGGTGCACGGGCTGTACATCCTGCACGAGGTTGACCTGACGACCGCGCA
CAAGGAAGCAGAGTGGGAATTTGAGCCCCTCGCCTGGCGGGTTTGGCTGGTGGTCTTCTACTTCGGCTGCTTGTCCT
TGACCGTCTGGCTGCTCGAGGGGAGTTACGGTGGATCGGACCACCACGCCGCGCGAGCCCAAAGTCCAGATGTCCGC
GCGCGGCGGTCGGAGCTTGATGACAACATCGCGCAGATGGGAGCTGTCCATGGTCTGGAGCTCCCGCGGCGTCAGGT
CAGGCGGGAGCTCCTGCAGGTTTACCTCGCATAGACGGGTCAGGGCGCGGGCTAGATCCAGGTGATACCTAATTTCC
AGGGGCTGGTTGGTGGCGGCGTCGATGGCTTGCAAGAGGCCGCATCCCCGCGGCGCGACTACGGTACCGCGCGGCGG
GCGGTGGGCCGCGGGGGTGTCCTTGGATGATGCATCTAAAAGCGGTGACGCGGGCGAGCCCCCGGAGGTAGGGGGGG
CTCCGGACCCGCCGGGAGAGGGGGCAGGGGCACGTCGGCGCCGCGCGCGGGCAGGAGCTGGTGCTGCGCGCGTAGGT
TGCTGGCGAACGCGACGACGCGGCGGTTGATCTCCTGAATCTGGCGCCTCTGCGTGAAGACGACGGGCCCGGTGAGC
TTGAGCCTGAAAGAGAGTTCGACAGAATCAATTTCGGTGTCGTTGACGGCGGCCTGGCGCAAAATCTCCTGCACGTC
TCCTGAGTTGTCTTGATAGGCGATCTCGGCCATGAACTGCTCGATCTCTTCCTCCTGGAGATCTCCGCGTCCGGCTC
GCTCCACGGTGGCGGCGAGGTCGTTGGAAATGCGGGCCATGAGCTGCGAGAAGGCGTTGAGGCCTCCCTCGTTCCAG
ACGCGGCTGTAGACCACGCCCCCTTCGGCATCGCGGGCGCGCATGACCACCTGCGCGAGATTGAGCTCCACGTGCCG
GGCGAAGACGGCGTAGTTTCGCAGGCGCTGAAAGAGGTAGTTGAGGGTGGTGGCGGTGTGTTCTGCCACGAAGAAGT
ACATAACCCAGCGTCGCAACGTGGATTCGTTGATATCCCCCAAGGCCTCAAGGCGCTCCATGGCCTCGTAGAAGTCC
ACGGCGAAGTTGAAAAACTGGGAGTTGCGCGCCGACACGGTTAACTCCTCCTCCAGAAGACGGATGAGCTCGGCGAC
AGTGTCGCGCACCTCGCGCTCAAAGGCTACAGGGGCCTCTTCTTCTTCTTCAATCTCCTCTTCCATAAGGGCCTCCC
CTTCTTCTTCTTCTGGCGGCGGTGGGGGAGGGGGGACACGGCGGCGACGACGGCGCACCGGGAGGCGGTCGACAAAG
CGCTCGATCATCTCCCCGCGGCGACGGCGCATGGTCTCGGTGACGGCGCGGCCGTTCTCGCGGGGGCGCAGTTGGAA
GACGCCGCCCGTCATGTCCCGGTTATGGGTTGGCGGGGGGCTGCCATGCGGCAGGGATACGGCGCTAACGATGCATC
TCAACAATTGTTGTGTAGGTACTCCGCCGCCGAGGGACCTGAGCGAGTCCGCATCGACCGGATCGGAAAACCTCTCG
AGAAAGGCGTCTAACCAGTCACAGTCGCAAGGTAGGCTGAGCACCGTGGCGGGCGGCAGCGGGCGGCGGTCGGGGTT
GTTTCTGGCGGAGGTGCTGCTGATGATGTAATTAAAGTAGGCGGTCTTGAGACGGCGGATGGTCGACAGAAGCACCA
TGTCCTTGGGTCCGGCCTGCTGAATGCGCAGGCGGTCGGCCATGCCCCAGGCTTCGTTTTGACATCGGCGCAGGTCT
TTGTAGTAGTCTTGCATGAGCCTTTCTACCGGCACTTCTTCTTCTCCTTCCTCTTGTCCTGCATCTCTTGCATCTAT
CGCTGCGGCGGCGGCGGAGTTTGGCCGTAGGTGGCGCCCTCTTCCTCCCATGCGTGTGACCCCGAAGCCCCTCATCG
GCTGAAGCAGGGCTAGGTCGGCGACAACGCGCTCGGCTAATATGGCCTGCTGCACCTGCGTGAGGGTAGACTGGAAG
TCATCCATGTCCACAAAGCGGTGGTATGCGCCCGTGTTGATGGTGTAAGTGCAGTTGGCCATAACGGACCAGTTAAC
GGTCTGGTGACCCGGCTGCGAGAGCTCGGTGTACCTGAGACGCGAGTAAGCCCTCGAGTCAAATACGTAGTCGTTGC
AAGTCCGCACCAGGTACTGGTATCCCACCAAAAAGTGCGGCGGCGGCTGGCGGTAGAGGGGCCAGCGTAGGGTGGCC
GGGGCTCCGGGGGCGAGATCTTCCAACATAAGGCGATGATATCCGTAGATGTACCTGGACATCCAGGTGATGCCGGC
GGCGGTGGTGGAGGCGCGCGGAAAGTCGCGGACGCGGTTCCAGATGTTGCGCAGCGGCAAAAAGTGCTCCATGGTCG
GGACGCTCTGGCCGGTCAGGCGCGCGCAATCGTTGACGCTCTAGACCGTGCAAAAGGAGAGCCTGTAAGCGGGCACT
CTTCCGTGGTCTGGTGGATAAATTCGCAAGGGTATCATGGCGGACGACCGGGGTTCGAGCCCCGTATCCGGCCGTCC
GCCGTGATCCATGCGGTTACCGCCCGCGTGTCGAACCCAGGTGTGCGACGTCAGACAACGGGGGAGTGCTCCTTTTG
GCTTCCTTCCAGGCGCGGCGGCTGCTGCGCTAGCTTTTTTGGCCACTGGCCGCGCGCAGCGTAAGCGGTTAGGCTGG
AAAGCGAAAGCATTAAGTGGCTCGCTCCCTGTAGCCGGAGGGTTATTTTCCAAGGGTTGAGTCGCGGGACCCCCGGT
TCGAGTCTCGGACCGGCCGGACTGCGGCGAACGGGGGTTTGCCTCCCCGTCATGCAAGACCCCGCTTGCAAATTCCT
CCGGAAACAGGGACGAGCCCCTTTTTTGCTTTTCCCAGATGCATCCGGTGCTGCGGCAGATGCGCCCCCCTCCTCAG
CAGCGGCAAGAGCAAGAGCAGCGGCAGACATGCAGGGCACCCTCCCCTCCTCCTACCGCGTCAGGAGGGGCGACATC
CGCGGTTGACGCGGCAGCAGATGGTGATTACGAACCCCCGCGGCGCCGGGCCCGGCACTACCTGGACTTGGAGGAGG
GCGAGGGCCTGGCGCGGCTAGGAGCGCCCTCTCCTGAGCGGTACCCAAGGGTGCAGCTGAAGCGTGATACGCGTGAG
GCGTACGTGCCGCGGCAGAACCTGTTTCGCGACCGCGAGGGAGAGGAGCCCGAGGAGATGCGGGATCGAAAGTTCCA
CGCAGGGCGCGAGCTGCGGCATGGCCTGAATCGCGAGCGGTTGCTGCGCGAGGAGGACTTTGAGCCCGACGCGCGAA
CCGGGATTAGTCCCGCGCGCGCACACGTGGCGGCCGCCGACCTGGTAACCGCATACGAGCAGACGGTGAACCAGGAG
ATTAACTTTCAAAAAAGCTTTAACAACCACGTGCGTACGCTTGTGGCGCGCGAGGAGGTGGCTATAGGACTGATGCA
TCTGTGGGACTTTGTAAGCGCGCTGGAGCAAAACCCAAATAGCAAGCCGCTCATGGCGCAGCTGTTCCTTATAGTGC
AGCACAGCAGGGACAACGAGGCATTCAGGGATGCGCTGCTAAACATAGTAGAGCCCGAGGGCCGCTGGCTGCTCGAT
TTGATAAACATCCTGCAGAGCATAGTGGTGCAGGAGCGCAGCTTGAGCCTGGCTGACAAGGTGGCCGCCATCAACTA
TTCCATGCTTAGCCTGGGCAAGTTTTACGCCCGCAAGATATACCATACCCCTTACGTTCCCATAGACAAGGAGGTAA
AGATCGAGGGGTTCTACATGCGCATGGCGCTGAAGGTGCTTACCTTGAGCGACGACCTGGGCGTTTATCGCAACGAG
CGCATCCACAAGGCCGTGAGCGTGAGCCGGCGGCGCGAGCTCAGCGACCGCGAGCTGATGCACAGCCTGCAAAGGGC
CCTGGCTGGCACGGGCAGCGGCGATAGAGAGGCCGAGTCCTACTTTGACGCGGGCGCTGACCTGCGCTGGGCCCCAA
GCCGACGCGCCCTGGAGGCAGCTGGGGCCGGACCTGGGCTGGCGGTGGCACCCGCGCGCGCTGGCAACGTCGGCGGC
GTGGAGGAATATGACGAGGACGATGAGTACGAGCCAGAGGACGGCGAGTACTAAGCGGTGATGTTTCTGATCAGATG
ATGCAAGACGCAACGGACCCGGCGGTGCGGGCGGCGCTGCAGAGCCAGCCGTCCGGCCTTAACTCCACGGACGACTG
GCGCCAGGTCATGGACCGCATCATGTCGCTGACTGCGCGCAATCCTGACGCGTTCCGGCAGCAGCCGCAGGCCAACC
GGCTCTCCGCAATTCTGGAAGCGGTGGTCCCGGCGCGCGCAAACCCCACGCACGAGAAGGTGCTGGCGATCGTAAAC
GCGCTGGCCGAAAACAGGGCCATCCGGCCCGACGAGGCCGGCCTGGTCTACGACGCGCTGCTTCAGCGCGTGGCTCG
TTACAACAGCGGCAACGTGCAGACCAACCTGGACCGGCTGGTGGGGGATGTGCGCGAGGCCGTGGCGCAGCGTGAGC
GCGCGCAGCAGCAGGGCAACCTGGGCTCCATGGTTGCACTAAACGCCTTCCTGAGTACACAGCCCGCCAACGTGCCG
CGGGGACAGGAGGACTACACCAACTTTGTGAGCGCACTGCGGCTAATGGTGACTGAGACACCGCAAAGTGAGGTGTA
CCAGTCTGGGCCAGACTATTTTTTCCAGACCAGTAGACAAGGCCTGCAGACCGTAAACCTGAGCCAGGCTTTCAAAA
ACTTGCAGGGGCTGTGGGGGGTGCGGGCTCCCACAGGCGACCGCGCGACCGTGTCTAGCTTGCTGACGCCCAACTCG
CGCCTGTTGCTGCTGCTAATAGCGCCCTTCACGGACAGTGGCAGCGTGTCCCGGGACACATACCTAGGTCACTTGCT
GACACTGTACCGCGAGGCCATAGGTCAGGCGCATGTGGACGAGCATACTTTCCAGGAGATTACAAGTGTCAGCCGCG
CGCTGGGGCAGGAGGACACGGGCAGCCTGGAGGCAACCCTAAACTACCTGCTGACCAACCGGCGGCAGAAGATCCCC
TCGTTGCACAGTTTAAACAGCGAGGAGGAGCGCATTTTGCGCTACGTGCAGCAGAGCGTGAGCCTTAACCTGATGCG
CGACGGGGTAACGCCCAGCGTGGCGCTGGACATGACCGCGCGCAACATGGAACCGGGCATGTATGCCTCAAACCGGC
CGTTTATCAACCGCCTAATGGACTACTTGCATCGCGCGGCCGCCGTGAACCCCGAGTATTTCACCAATGCCATCTTG
AACCCGCACTGGCTACCGCCCCCTGGTTTCTACACCGGGGGATTCGAGGTGCCCGAGGGTAACGATGGATTCCTCTG
GGACGACATAGACGACAGCGTGTTTTCCCCGCAACCGCAGACCCTGCTAGAGTTGCAACAGCGCGAGCAGGCAGAGG
CGGCGCTGCGAAAGGAAAGCTTCCGCAGGCCAAGCAGCTTGTCCGATCTAGGCGCTGCGGCCCCGCGGTCAGATGCT
AGTAGCCCATTTCCAAGCTTGATAGGGTCTCTTACCAGCACTCGCACCACCCGCCCGCGCCTGCTGGGCGAGGAGGA
GTACCTAAACAACTCGCTGCTGCAGCCGCAGCGCGAAAAAAACCTGCCTCCGGCATTTCCCAACAACGGGATAGAGA
GCCTAGTGGACAAGATGAGTAGATGGAAGACGTACGCGCAGGAGCACAGGGACGTGCCAGGCCCGCGCCCGCCCACC
CGTCGTCAAAGGCACGACCGTCAGCGGGGTCTGGTGTGGGAGGACGATGACTCGGCAGACGACAGCAGCGTCCTGGA
TTTGGGAGGGAGTGGCAACCCGTTTGCGCACCTTCGCCCCAGGCTGGGGAGAATGTTTTAAAAAAAAAAAAGCATGA
TGCAAAATAAAAAACTCACCAAGGCCATGGCACCGAGCGTTGGTTTTCTTGTATTCCCCTTAGTATGCGGCGCGCGG
CGATGTATGAGGAAGGTCCTCCTCCCTCCTACGAGAGTGTGGTGAGCGCGGCGCCAGTGGCGGCGGCGCTGGGTTCT
CCCTTCGATGCTCCCCTGGACCCGCCGTTTGTGCCTCCGCGGTACCTGCGGCCTACCGGGGGGAGAAACAGCATCCG
TTACTCTGAGTTGGCACCCCTATTCGACACCACCCGTGTGTACCTGGTGGACAACAAGTCAACGGATGTGGCATCCC
TGAACTACCAGAACGACCACAGCAACTTTCTGACCACGGTCATTCAAAACAATGACTACAGCCCGGGGGAGGCAAGC
ACACAGACCATCAATCTTGACGACCGGTCGCACTGGGGCGGCGACCTGAAAACCATCCTGCATACCAACATGCCAAA
TGTGAACGAGTTCATGTTTACCAATAAGTTTAAGGCGCGGGTGATGGTGTCGCGCTTGCCTACTAAGGACAATCAGG
TGGAGCTGAAATACGAGTGGGTGGAGTTCACGCTGCCCGAGGGCAACTACTCCGAGACCATGACCATAGACCTTATG
AACAACGCGATCGTGGAGCACTACTTGAAAGTGGGCAGACAGAACGGGGTTCTGGAAAGCGACATCGGGGTAAAGTT
TGACACCCGCAACTTCAGACTGGGGTTTGACCCCGTCACTGGTCTTGTCATGCCTGGGGTATATACAAACGAAGCCT
TCCATCCAGACATCATTTTGCTGCCAGGATGCGGGGTGGACTTCACCCACAGCCGCCTGAGCAACTTGTTGGGCATC
CGCAAGCGGCAACCCTTCCAGGAGGGCTTTAGGATCACCTACGATGATCTGGAGGGTGGTAACATTCCCGCACTGTT
GGATGTGGACGCCTACCAGGCGAGCTTGAAAGATGACACCGAACAGGGCGGGGGTGGCGCAGGCGGCAGCAACAGCA
GTGGCAGCGGCGCGGAAGAGAACTCCAACGCGGCAGCCGCGGCAATGCAGCCGGTGGAGGACATGAACGATCATGCC
ATTCGCGGCGACACCTTTGCCACACGGGCTGAGGAGAAGCGCGCTGAGGCCGAAGCAGCGGCCGAAGCTGCCGCCCC
CGCTGCGCAACCCGAGGTCGAGAAGCCTCAGAAGAAACCGGTGATCAAACCCCTGACAGAGGACAGCAAGAAACGCA
GTTACAACCTAATAAGCAATGACAGCACCTTCACCCAGTACCGCAGCTGGTACCTTGCATACAACTACGGCGACCCT
CAGACCGGAATCCGCTCATGGACCCTGCTTTGCACTCCTGACGTAACCTGCGGCTCGGAGCAGGTCTACTGGTCGTT
GCCAGACATGATGCAAGACCCCGTGACCTTCCGCTCCACGCGCCAGATCAGCAACTTTCCGGTGGTGGGCGCCGAGC
TGTTGCCCGTGCACTCCAAGAGCTTCTACAACGACCAGGCCGTCTACTCCCAACTCATCCGCCAGTTTACCTCTCTG
ACCCACGTGTTCAATCGCTTTCCCGAGAACCAGATTTTGGCGCGCCCGCCAGCCCCCACCATCACCACCGTCAGTGA
AAACGTTCCTGCTCTCACAGATCACGGGACGCTACCGCTGCGCAACAGCATCGGAGGAGTCCAGCGAGTGACCATTA
CTGACGCCAGACGCCGCACCTGCCCCTACGTTTACAAGGCCCTGGGCATAGTCTCGCCGCGCGTCCTATCGAGCCGC
ACTTTTTGAGCAAGCATGTCCATCCTTATATCGCCCAGCAATAACACAGGCTGGGGCCTGCGCTTCCCAAGCAAGAT
GTTTGGCGGGGCCAAGAAGCGCTCCGACCAACACCCAGTGCGCGTGCGCGGGCACTACCGCGCGCCCTGGGGCGCGC
ACAAACGCGGCCGCACTGGGCGCACCACCGTCGATGACGCCATCGACGCGGTGGTGGAGGAGGCGCGCAACTACACG
CCCACGCCGCCACCAGTGTCCACAGTGGACGCGGCCATTCAGACCGTGGTGCGCGGAGCCCGGCGCTATGCTAAAAT
GAAGAGACGGCGGAGGCGCGTAGCACGTCGCCACCGCCGCCGACCCGGCACTGCCGCCCAACGCGCGGCGGCGGCCC
TGCTTAACCGCGCACGTCGCACCGGCCGACGGGCGGCCATGCGGGCCGCTCGAAGGCTGGCCGCGGGTATTGTCACT
GTGCCCCCCAGGTCCAGGCGACGAGCGGCCGCCGCAGCAGCCGCGGCCATTAGTGCTATGACTCAGGGTCGCAGGGG
CAACGTGTATTGGGTGCGCGACTCGGTTAGCGGCCTGCGCGTGCCCGTGCGCACCCGCCCCCCGCGCAACTAGATTG
CAAGAAAAAACTACTTAGACTCGTACTGTTGTATGTATCCAGCGGCGGCGGCGCGCAACGAAGCTATGTCCAAGCGC
AAAATCAAAGAAGAGATGCTCCAGGTCATCGCGCCGGAGATCTATGGCCCCCCGAAGAAGGAAGAGCAGGATTACAA
GCCCCGAAAGCTAAAGCGGGTCAAAAAGAAAAAGAAAGATGATGATGATGAACTTGACGACGAGGTGGAACTGCTGC
ACGCTACCGCGCCCAGGCGACGGGTACAGTGGAAAGGTCGACGCGTAAAACGTGTTTTGCGACCCGGCACCACCGTA
GTCTTTACGCCCGGTGAGCGCTCCACCCGCACCTACAAGCGCGTGTATGATGAGGTGTACGGCGACGAGGACCTGCT
TGAGCAGGCCAACGAGCGCCTCGGGGAGTTTGCCTACGGAAAGCGGCATAAGGACATGCTGGCGTTGCCGCTGGACG
AGGGCAACCCAACACCTAGCCTAAAGCCCGTAACACTGCAGCAGGTGCTGCCCGCGCTTGCACCGTCCGAAGAAAAG
CGCGGCCTAAAGCGCGAGTCTGGTGACTTGGCACCCACCGTGCAGCTGATGGTACCCAAGCGCCAGCGACTGGAAGA
TGTCTTGGAAAAAATGACCGTGGAACCTGGGCTGGAGCCCGAGGTCCGCGTGCGGCCAATCAAGCAGGTGGCGCCGG
GACTGGGCGTGCAGACCGTGGACGTTCAGATACCCACTACCAGTAGCACCAGTATTGCCACCGCCACAGAGGGCATG
GAGACACAAACGTCCCCGGTTGCCTCAGCGGTGGCGGATGCCGCGGTGCAGGCGGTCGCTGCGGCCGCGTCCAAGAC
CTCTACGGAGGTGCAAACGGACCCGTGGATGTTTCGCGTTTCAGCCCCCCGGCGCCCGCGCGGTTCGAGGAAGTACG
GCGCCGCCAGCGCGCTACTGCCCGAATATGCCCTACATCCTTCCATTGCGCCTACCCCCGGCTATCGTGGCTACACC
TACCGCCCCAGAAGACGAGCAACTACCCGACGCCGAACCACCACTGGAACCCGCCGCCGCCGTCGCCGTCGCCAGCC
CGTGCTGGCCCCGATTTCCGTGCGCAGGGTGGCTCGCGAAGGAGGCAGGACCCTGGTGCTGCCAACAGCGCGCTACC
ACCCCAGCATCGTTTAAAAGCCGGTCTTTGTGGTTCTTGCAGATATGGCCCTCACCTGCCGCCTCCGTTTCCCGGTG
CCGGGATTCCGAGGAAGAATGCACCGTAGGAGGGGCATGGCCGGCCACGGCCTGACGGGCGGCATGCGTCGTGCGCA
CCACCGGCGGCGGCGCGCGTCGCACCGTCGCATGCGCGGCGGTATCCTGCCCCTCCTTATTCCACTGATCGCCGCGG
CGATTGGCGCCGTGCCCGGAATTGCATCCGTGGCCTTGCAGGCGCAGAGACACTGATTAAAAACAAGTTGCATGTGG
AAAAATCAAAATAAAAAGTCTGGACTCTCACGCTCGCTTGGTCCTGTAACTATTTTGTAGAATGGAAGACATCAACT
TTGCGTCTCTGGCCCCGCGACACGGCTCGCGCCCGTTCATGGGAAACTGGCAAGATATCGGCACCAGCAATATGAGC
GGTGGCGCCTTCAGCTGGGGCTCGCTGTGGAGCGGCATTAAAAATTTCGGTTCCACCGTTAAGAACTATGGCAGCAA
GGCCTGGAACAGCAGCACAGGCCAGATGCTGAGGGATAAGTTGAAAGAGCAAAATTTCCAACAAAAGGTGGTAGATG
GCCTGGCCTCTGGCATTAGCGGGGTGGTGGACCTGGCCAACCAGGCAGTGCAAAATAAGATTAACAGTAAGCTTGAT
CCCCGCCCTCCCGTAGAGGAGCCTCCACCGGCCGTGGAGACAGTGTCTCCAGAGGGGCGTGGCGAAAAGCGTCCGCG
CCCCGACAGGGAAGAAACTCTGGTGACGCAAATAGACGAGCCTCCCTCGTACGAGGAGGCACTAAAGCAAGGCCTGC
CCACCACCCGTCCCATCGCGCCCATGGCTACCGGAGTGCTGGGCCAGCACACACCCGTAACGCTGGACCTGCCTCCC
CCCGCCGACACCCAGCAGAAACCTGTGCTGCCAGGCCCGACCGCCGTTGTTGTAACCCGTCCTAGCCGCGCGTCCCT
GCGCCGCGCCGCCAGCGGTCCGCGATCGTTGCGGCCCGTAGCCAGTGGCAACTGGCAAAGCACACTGAACAGCATCG
TGGGTCTGGGGGTGCAATCCCTGAAGCGCCGACGATGCTTCTGAATAGCTAACGTGTCGTATGTGTGTCATGTATGC
GTCCATGTCGCCGCCAGAGGAGCTGCTGAGCCGCCGCGCGCCCGCTTTCCAAGATGGCTACCCCTTCGATGATGCCG
CAGTGGTCTTACATGCACATCTCGGGCCAGGACGCCTCGGAGTACCTGAGCCCCGGGCTGGTGCAGTTTGCCCGCGC
CACCGAGACGTACTTCAGCCTGAATAACAAGTTTAGAAACCCCACGGTGGCGCCTACGCACGACGTGACCACAGACC
GGTCCCAGCGTTTGACGCTGCGGTTCATCCCTGTGGACCGTGAGGATACTGCGTACTCGTACAAGGCGCGGTTCACC
CTAGCTGTGGGTGATAACCGTGTGCTGGACATGGCTTCCACGTACTTTGACATCCGCGGCGTGCTGGACAGGGGCCC
TACTTTTAAGCCCTACTCTGGCACTGCCTACAACGCCCTGGCTCCCAAGGGTGCCCCAAATCCTTGCGAATGGGATG
AAGCTGCTACTGCTCTTGAAATAAACCTAGAAGAAGAGGACGATGACAACGAAGACGAAGTAGACGAGCAAGCTGAG
CAGCAAAAAACTCACGTATTTGGGCAGGCGCCTTATTCTGGTATAAATATTACAAAGGAGGGTATTCAAATAGGTGT
CGAAGGTCAAACACCTAAATATGCCGATAAAACATTTCAACCTGAACCTCAAATAGGAGAATCTCAGTGGTACGAAA
CTGAAATTAATCATGCAGCTGGGAGAGTCCTTAAAAAGACTACCCCAATGAAACCATGTTACGGTTCATATGCAAAA
CCCACAAATGAAAATGGAGGGCAAGGCATTCTTGTAAAGCAACAAAATGGAAAGCTAGAAAGTCAAGTGGAAATGCA
ATTTTTCTCAACTACTGAGGCGACCGCAGGCAATGGTGATAACTTGACTCCTAAAGTGGTATTGTACAGTGAAGATG
TAGATATAGAAACCCCAGACACTCATATTTCTTACATGCCCACTATTAAGGAAGGTAACTCACGAGAACTAATGGGC
CAACAATCTATGCCCAACAGGCCTAATTACATTGCTTTTAGGGACAATTTTATTGGTCTAATGTATTACAACAGCAC
GGGTAATATGGGTGTTCTGGCGGGCCAAGCATCGCAGTTGAATGCTGTTGTAGATTTGCAAGACAGAAACACAGAGC
TTTCATACCAGCTTTTGCTTGATTCCATTGGTGATAGAACCAGGTACTTTTCTATGTGGAATCAGGCTGTTGACAGC
TATGATCCAGATGTTAGAATTATTGAAAATCATGGAACTGAAGATGAACTTCCAAATTACTGCTTTCCACTGGGAGG
TGTGATTAATACAGAGACTCTTACCAAGGTAAAACCTAAAACAGGTCAGGAAAATGGATGGGAAAAAGATGCTACAG
AATTTTCAGATAAAAATGAAATAAGAGTTGGAAATAATTTTGCCATGGAAATCAATCTAAATGCCAACCTGTGGAGA
AATTTCCTGTACTCCAACATAGCGCTGTATTTGCCCGACAAGCTAAAGTACAGTCCTTCCAACGTAAAAATTTCTGA
TAACCCAAACACCTACGACTACATGAACAAGCGAGTGGTGGCTCCCGGGTTAGTGGACTGCTACATTAACCTTGGAG
CACGCTGGTCCCTTGACTATATGGACAACGTCAACCCATTTAACCACCACCGCAATGCTGGCCTGCGCTACCGCTCA
ATGTTGCTGGGCAATGGTCGCTATGTGCCCTTCCACATCCAGGTGCCTCAGAAGTTCTTTGCCATTAAAAACCTCCT
TCTCCTGCCGGGCTCATACACCTACGAGTGGAACTTCAGGAAGGATGTTAACATGGTTCTGCAGAGCTCCCTAGGAA
ATGACCTAAGGGTTGACGGAGCCAGCATTAAGTTTGATAGCATTTGCCTTTACGCCACCTTCTTCCCCATGGCCCAC
AACACCGCCTCCACGCTTGAGGCCATGCTTAGAAACGACACCAACGACCAGTCCTTTAACGACTATCTCTCCGCCGC
CAACATGCTCTACCCTATACCCGCCAACGCTACCAACGTGCCCATATCCATCCCCTCCCGCAACTGGGCGGCTTTCC
GCGGCTGGGCCTTCACGCGCCTTAAGACTAAGGAAACCCCATCACTGGGCTCGGGCTACGACCCTTATTACACCTAC
TCTGGCTCTATACCCTACCTAGATGGAACCTTTTACCTCAACCACACCTTTAAGAAGGTGGCCATTACCTTTGACTC
TTCTGTCAGCTGGCCTGGCAATGACCGCCTGCTTACCCCCAACGAGTTTGAAATTAAGCGCTCAGTTGACGGGGAGG
GTTACAACGTTGCCCAGTGTAACATGACCAAAGACTGGTTCCTGGTACAAATGCTAGCTAACTACAACATTGGCTAC
CAGGGCTTCTATATCCCAGAGAGCTACAAGGACCGCATGTACTCCTTCTTTAGAAACTTCCAGCCCATGAGCCGTCA
GGTGGTGGATGATACTAAATACAAGGACTACCAACAGGTGGGCATCCTACACCAACACAACAACTCTGGATTTGTTG
GCTACCTTGCCCCCACCATGCGCGAAGGACAGGCCTACCCTGCTAACTTCCCCTATCCGCTTATAGGCAAGACCGCA
GTTGACAGCATTACCCAGAAAAAGTTTCTTTGCGATCGCACCCTTTGGCGCATCCCATTCTCCAGTAACTTTATGTC
CATGGGCGCACTCACAGACCTGGGCCAAAACCTTCTCTACGCCAACTCCGCCCACGCGCTAGACATGACTTTTGAGG
TGGATCCCATGGACGAGCCCACCCTTCTTTATGTTTTGTTTGAAGTCTTTGACGTGGTCCGTGTGCACCGGCCGCAC
CGCGGCGTCATCGAAACCGTGTACCTGCGCACGCCCTTCTCGGCCGGCAACGCCACAACATAAAGAAGCAAGCAACA
TCAACAACAGCTGCCGCCATGGGCTCCAGTGAGCAGGAACTGAAAGCCATTGTCAAAGATCTTGGTTGTGGGCCATA
TTTTTTGGGCACCTATGACAAGCGCTTTCCAGGCTTTGTTTCTCCACACAAGCTCGCCTGCGCCATAGTCAATACGG
CCGGTCGCGAGACTGGGGGCGTACACTGGATGGCCTTTGCCTGGAACCCGCACTCAAAAACATGCTACCTCTTTGAG
CCCTTTGGCTTTTCTGACCAGCGACTCAAGCAGGTTTACCAGTTTGAGTACGAGTCACTCCTGCGCCGTAGCGCCAT
TGCTTCTTCCCCCGACCGCTGTATAACGCTGGAAAAGTCCACCCAAAGCGTACAGGGGCCCAACTCGGCCGCCTGTG
GACTATTCTGCTGCATGTTTCTCCACGCCTTTGCCAACTGGCCCCAAACTCCCATGGATCACAACCCCACCATGAAC
CTTATTACCGGGGTACCCAACTCCATGCTCAACAGTCCCCAGGTACAGCCCACCCTGCGTCGCAACCAGGAACAGCT
CTACAGCTTCCTGGAGCGCCACTCGCCCTACTTCCGCAGCCACAGTGCGCAGATTAGGAGCGCCACTTCTTTTTGTC
ACTTGAAAAACATGTAAAAATAATGTACTAGAGACACTTTCAATAAAGGCAAATGCTTTTATTTGTACACTCTCGGG
TGATTATTTACCCCCACCCTTGCCGTCTGCGCCGTTTAAAAATCAAAGGGGTTCTGCCGCGCATCGCTATGCGCCAC
TGGCAGGGACACGTTGCGATACTGGTGTTTAGTGCTCCACTTAAACTCAGGCACAACCATCCGCGGCAGCTCGGTGA
AGTTTTCACTCCACAGGCTGCGCACCATCACCAACGCGTTTAGCAGGTCGGGCGCCGATATCTTGAAGTCGCAGTTG
GGGCCTCCGCCCTGCGCGCGCGAGTTGCGATACACAGGGTTGCAGCACTGGAACACTATCAGCGCCGGGTGGTGCAC
GCTGGCCAGCACGCTCTTGTCGGAGATCAGATCCGCGTCCAGGTCCTCCGCGTTGCTCAGGGCGAACGGAGTCAACT
TTGGTAGCTGCCTTCCCAAAAAGGGCGCGTGCCCAGGCTTTGAGTTGCACTCGCACCGTAGTGGCATCAAAAGGTGA
CCGTGCCCGGTCTGGGCGTTAGGATACAGCGCCTGCATAAAAGCCTTGATCTGCTTAAAAGCCACCTGAGCCTTTGC
GCCTTCAGAGAAGAACATGCCGCAAGACTTGCCGGAAAACTGATTGGCCGGACAGGCCGCGTCGTGCACGCAGCACC
TTGCGTCGGTGTTGGAGATCTGCACCACATTTCGGCCCCACCGGTTCTTCACGATCTTGGCCTTGCTAGACTGCTCC
TTCAGCGCGCGCTGCCCGTTTTCGCTCGTCACATCCATTTCAATCACGTGCTCCTTATTTATCATAATGCTTCCGTG
TAGACACTTAAGCTCGCCTTCGATCTCAGCGCAGCGGTGCAGCCACAACGCGCAGCCCGTGGGCTCGTGATGCTTGT
AGGTCACCTCTGCAAACGACTGCAGGTACGCCTGCAGGAATCGCCCCATCATCGTCACAAAGGTCTTGTTGCTGGTG
AAGGTCAGCTGCAACCCGCGGTGCTCCTCGTTCAGCCAGGTCTTGCATACGGCCGCCAGAGCTTCCACTTGGTCAGG
CAGTAGTTTGAAGTTCGCCTTTAGATCGTTATCCACGTGGTACTTGTCCATCAGCGCGCGCGCAGCCTCCATGCCCT
TCTCCCACGCAGACACGATCGGCACACTCAGCGGGTTCATCACCGTAATTTCACTTTCCGCTTCGCTGGGCTCTTCC
TCTTCCTCTTGCGTCCGCATACCACGCGCCACTGGGTCGTCTTCATTCAGCCGCCGCACTGTGCGCTTACCTCCTTT
GCCATGCTTGATTAGCACCGGTGGGTTGCTGAAACCCACCATTTGTAGCGCCACATCTTCTCTTTCTTCCTCGCTGT
CCACGATTACCTCTGGTGATGGCGGGCGCTCGGGCTTGGGAGAAGGGCGCTTCTTTTTCTTCTTGGGCGCAATGGCC
AAATCCGCCGCCGAGGTCGATGGCCGCGGGCTGGGTGTGCGCGGCACCAGCGCGTCTTGTGATGAGTCTTCCTCGTC
CTCGGACTCGATACGCCGCCTCATCCGCTTTTTTGGGGGCGCCCGGGGAGGCGGCGGCGACGGGGACGGGGACGACA
CGTCCTCCATGGTTGGGGGACGTCGCGCCGCACCGCGTCCGCGCTCGGGGGTGGTTTCGCGCTGCTCCTCTTCCCGA
CTGGCCATTTCCTTCTCCTATAGGCAGAAAAAGATCATGGAGTCAGTCGAGAAGAAGGACAGCCTAACCGCCCCCTC
TGAGTTCGCCACCACCGCCTCCACCGATGCCGCCAACGCGCCTACCACCTTCCCCGTCGAGGCACCCCCGCTTGAGG
AGGAGGAAGTGATTATCGAGCAGGACCCAGGTTTTGTAAGCGAAGACGACGAGGACCGCTCAGTACCAACAGAGGAT
AAAAAGCAAGACCAGGACAACGCAGAGGCAAACGAGGAACAAGTCGGGCGGGGGGACGAAAGGCATGGCGACTACCT
AGATGTGGGAGACGACGTGCTGTTGAAGCATCTGCAGCGCCAGTGCGCCATTATCTGCGACGCGTTGCAAGAGCGCA
GCGATGTGCCCCTCGCCATAGCGGATGTCAGCCTTGCCTACGAACGCCACCTATTCTCACCGCGCGTACCCCCCAAA
CGCCAAGAAAACGGCACATGCGAGCCCAACCCGCGCCTCAACTTCTACCCCGTATTTGCCGTGCCAGAGGTGCTTGC
CACCTATCACATCTTTTTCCAAAACTGCAAGATACCCCTATCCTGCCGTGCCAACCGCAGCCGAGCGGACAAGCAGC
TGGCCTTGCGGCAGGGCGCTGTCATACCTGATATCGCCTCGCTCAACGAAGTGCCAAAAATCTTTGAGGGTCTTGGA
CGCGACGAGAAGCGCGCGGCAAACGCTCTGCAACAGGAAAACAGCGAAAATGAAAGTCACTCTGGAGTGTTGGTGGA
ACTCGAGGGTGACAACGCGCGCCTAGCCGTACTAAAACGCAGCATCGAGGTCACCCACTTTGCCTACCCGGCACTTA
ACCTACCCCCCAAGGTCATGAGCACAGTCATGAGTGAGCTGATCGTGCGCCGTGCGCAGCCCCTGGAGAGGGATGCA
AATTTGCAAGAACAAACAGAGGAGGGCCTACCCGCAGTTGGCGACGAGCAGCTAGCGCGCTGGCTTCAAACGCGCGA
GCCTGCCGACTTGGAGGAGCGACGCAAACTAATGATGGCCGCAGTGCTCGTTACCGTGGAGCTTGAGTGCATGCAGC
GGTTCTTTGCTGACCCGGAGATGCAGCGCAAGCTAGAGGAAACATTGCACTACACCTTTCGACAGGGCTACGTACGC
CAGGCCTGCAAGATCTCCAACGTGGAGCTCTGCAACCTGGTCTCCTACCTTGGAATTTTGCACGAAAACCGCCTTGG
GCAAAACGTGCTTCATTCCACGCTCAAGGGCGAGGCGCGCCGCGACTACGTCCGCGACTGCGTTTACTTATTTCTAT
GCTACACCTGGCAGACGGCCATGGGCGTTTGGCAGCAGTGCTTGGAGGAGTGCAACCTCAAGGAGCTGCAGAAACTG
CTAAAGCAAAACTTGAAGGACCTATGGACGGCCTTCAACGAGCGCTCCGTGGCCGCGCACCTGGCGGACATCATTTT
CCCCGAACGCCTGCTTAAAACCCTGCAACAGGGTCTGCCAGACTTCACCAGTCAAAGCATGTTGCAGAACTTTAGGA
ACTTTATCCTAGAGCGCTCAGGAATCTTGCCCGCCACCTGCTGTGCACTTCCTAGCGACTTTGTGCCCATTAAGTAC
CGCGAATGCCCTCCGCCGCTTTGGGGCCACTGCTACCTTCTGCAGCTAGCCAACTACCTTGCCTACCACTCTGACAT
AATGGAAGACGTGAGCGGTGACGGTCTACTGGAGTGTCACTGTCGCTGCAACCTATGCACCCCGCACCGCTCCCTGG
TTTGCAATTCGCAGCTGCTTAACGAAAGTCAAATTATCGGTACCTTTGAGCTGCAGGGTCCCTCGCCTGACGAAAAG
TCCGCGGCTCCGGGGTTGAAACTCACTCCGGGGCTGTGGACGTCGGCTTACCTTCGCAAATTTGTACCTGAGGACTA
CCACGCCCACGAGATTAGGTTCTACGAAGACCAATCCCGCCCGCCAAATGCGGAGCTTACCGCCTGCGTCATTACCC
AGGGCCACATTCTTGGCCAATTGCAAGCCATCAACAAAGCCCGCCAAGAGTTTCTGCTACGAAAGGGACGGGGGGTT
TACTTGGACCCCCAGTCCGGCGAGGAGCTCAACCCAATCCCCCCGCCGCCGCAGCCCTATCAGCAGCAGCCGCGGGC
CCTTGCTTCCCAGGATGGCACCCAAAAAGAAGCTGCAGCTGCCGCCGCCACCCACGGACGAGGAGGAATACTGGGAC
AGTCAGGCAGAGGAGGTTTTGGACGAGGAGGAGGAGGACATGATGGAAGACTGGGAGAGCCTAGACGAGGAAGCTTC
CGAGGTCGAAGAGGTGTCAGACGAAACACCGTCACCCTCGGTCGCATTCCCCTCGCCGGCGCCCCAGAAATCGGCAA
CCGGTTCCAGCATGGCTACAACCTCCGCTCCTCAGGCGCCGCCGGCACTGCCCGTTCGCCGACCCAACCGTAGATGG
GACACCACTGGAACCAGGGCCGGTAAGTCCAAGCAGCCGCCGCCGTTAGCCCAAGAGCAACAACAGCGCCAAGGCTA
CCGCTCATGGCGCGGGCACAAGAACGCCATAGTTGCTTGCTTGCAAGACTGTGGGGGCAACATCTCCTTCGCCCGCC
GCTTTCTTCTCTACCATCACGGCGTGGCCTTCCCCCGTAACATCCTGCATTACTACCGTCATCTCTACAGCCCATAC
TGCACCGGCGGCAGCGGCAGCGGCAGCAACAGCAGCGGCCACACAGAAGCAAAGGCGACCGGATAGCAAGACTCTGA
CAAAGCCCAAGAAATCCACAGCGGCGGCAGCAGCAGGAGGAGGAGCGCTGCGTCTGGCGCCCAACGAACCCGTATCG
ACCCGCGAGCTTAGAAACAGGATTTTTCCCACTCTGTATGCTATATTTCAACAGAGCAGGGGCCAAGAACAAGAGCT
GAAAATAAAAAACAGGTCTCTGCGATCCCTCACCCGCAGCTGCCTGTATCACAAAAGCGAAGATCAGCTTCGGCGCA
CGCTGGAAGACGCGGAGGCTCTCTTCAGTAAATACTGCGCGCTGACTCTTAAGGACTAGTTTCGCGCCCTTTCTCAA
ATTTAAGCGCGAAAACTACGTCATCTCCAGCGGCCACACCCGGCGCCAGCACCTGTCGTCAGCGCCATTATGAGCAA
GGAAATTCCCACGCCCTACATGTGGAGTTACCAGCCACAAATGGGACTTGCGGCTGGAGCTGCCCAAGACTACTCAA
CCCGAATAAACTACATGAGCGCGGGACCCCACATGATATCCCGGGTCAACGGAATCCGCGCCCACCGAAACCGAATT
CTCTTGGAACAGGCGGCTATTACCACCACACCTCGTAATAACCTTAATCCCCGTAGTTGGCCCGCTGCCCTGGTGTA
CCAGGAAAGTCCCGCTCCCACCACTGTGGTACTTCCCAGAGACGCCCAGGCCGAAGTTCAGATGACTAACTCAGGGG
CGCAGCTTGCGGGCGGCTTTCGTCACAGGGTGCGGTCGCCCGGGCAGGGTATAACTCACCTGACAATCAGAGGGCGA
GGTATTCAGCTCAACGACGAGTCGGTGAGCTCCTCGCTTGGTCTCCGTCCGGACGGGACATTTCAGATCGGCGGCGC
CGGCCGTCCTTCATTCACGCCTCGTCAGGCAATCCTAACTCTGCAGACCTCGTCCTCTGAGCCGCGCTCTGGAGGCA
TTGGAACTCTGCAATTTATTGAGGAGTTTGTGCCATCGGTCTACTTTAACCCCTTCTCGGGACCTCCCGGCCACTAT
CCGGATCAATTTATTCCTAACTTTGACGCGGTAAAGGACTCGGCGGACGGCTACGACTGAATGTTAAGTGGAGAGGC
AGAGCAACTGCGCCTGAAACACCTGGTCCACTGTCGCCGCCACAAGTGCTTTGCCCGCGACTCCGGTGAGTTTTGCT
ACTTTGAATTGCCCGAGGATCATATCGAGGGCCCGGCGCACGGCGTCCGGCTTACCGCCCAGGGAGAGCTTGCCCGT
AGCCTGATTCGGGAGTTTACCCAGCGCCCCCTGCTAGTTGAGCGGGACAGGGGACCCTGTGTTCTCACTGTGATTTG
CAACTGTCCTAACCTTGGATTACATCAAGATCTTTGTTGCCATCTCTGTGCTGAGTATAATAAATACAGAAATTAAA
ATATACTGGGGCTCCTATCGCCATCCTGTAAACGCCACCGTCTTCACCCGCCCAAGCAAACCAAGGCGAACCTTACC
TGGTACTTTTAACATCTCTCCCTCTGTGATTTACAACAGTTTCAACCCAGACGGAGTGAGTCTACGAGAGAACCTCT
CCGAGCTCAGCTACTCCATCAGAAAAAACACCACCCTCCTTACCTGCCGGGAACGTACGAGTGCGTCACCGGCCGCT
GCACCACACCTACCGCCTGACCGTAAACCAGACTTTTTCCGGACAGACCTCAATAACTCTGTTTACCAGAACAGGAG
GTGAGCTTAGAAAACCCTTAGGGTATTAGGCCAAAGGCGCAGCTACTGTGGGGTTTATGAACAATTCAAGCAACTCT
ACGGGCTATTCTAATTCAGGTTTCTCTAGAATCGGGGTTGGGGTTATTCTCTGTCTTGTGATTCTCTTTATTCTTAT
ACTAACGCTTCTCTGCCTAAGGCTCGCCGCCTGCTGTGTGCACATTTGCATTTATTGTCAGCTTTTTAAACGCTGGG
GTCGCCACCCAAGATGATTAGGTACATAATCCTAGGTTTACTCACCCTTGCGTCAGCCCACGGTACCACCCAAAAGG
TGGATTTTAAGGAGCCAGCCTGTAATGTTACATTCGCAGCTGAAGCTAATGAGTGCACCACTCTTATAAAATGCACC
ACAGAACATGAAAAGCTGCTTATTCGCCACAAAAACAAAATTGGCAAGTATGCTGTTTATGCTATTTGGCAGCCAGG
TGACACTACAGAGTATAATGTTACAGTTTTCCAGGGTAAAAGTCATAAAACTTTTATGTATACTTTTCCATTTTATG
AAATGTGCGACATTACCATGTACATGAGCAAACAGTATAAGTIGTGGCCCCCACAAAATTGTGTGGAAAACACTGGC
ACTTTCTGCTGCACTGCTATGCTAATTACAGTGCTCGCTTTGGTCTGTACCCTACTCTATATTAAATACAAAAGCAG
ACGCAGCTTTATTGAGGAAAAGAAAATGCCTTAATTTACTAAGTTACAAAGCTAATGTCACCACTAACTGCTTTACT
CGCTGCTTGCAAAACAAATTCAAAAAGTTAGCATTATAATTAGAATAGGATTTAAACCCCCCGGTCATTTCCTGCTC
AATACCATTCCCCTGAACAATTGACTCTATGTGGGATATGCTCCAGCGCTACAACCTTGAAGTCAGGCTTCCTGGAT
GTCAGCATCTGACTTTGGCCAGCACCTGTCCCGCGGATTTGTTCCAGTCCAACTACAGCGACCCACCCTAACAGAGA
TGACCAACACAACCAACGCGGCCGCCGCTACCGGACTTACATCTACCACAAATACACCCCAAGTTTCTGCCTTTGTC
AATAACTGGGATAACTTGGGCATGTGGTGGTTCTCCATAGCGCTTATGTTTGTATGCCTTATTATTATGTGGCTCAT
CTGCTGCCTAAAGCGCAAACGCGCCCGACCACCCATCTATAGTCCCATCATTGTGCTACACCCAAACAATGATGGAA
TCCATAGATTGGACGGACTGAAACACATGTTCTTTTCTCTTACAGTATGATTAAATGAGACATGATTCCTCGAGTTT
TTATATTACTGACCCTTGTTGCGCTTTTTTGTGCGTGCTCCACATTGGCTGCGGTTTCTCACATCGAAGTAGACTGC
ATTCCAGCCTTCACAGTCTATTTGCTTTACGGATTTGTCACCCTCACGCTCATCTGCAGCCTCATCACTGTGGTCAT
CGCCTTTATCCAGTGCATTGACTGGGTCTGTGTGCGCTTTGCATATCTCAGACACCATCCCCAGTACAGGGACAGGA
CTATAGCTGAGCTTCTTAGAATTCTTTAATTATGAAATTTACTGTGACTTTTCTGCTGATTATTTGCACCCTATCTG
CGTTTTGTTCCCCGACCTCCAAGCCTCAAAGACATATATCATGCAGATTCACTCGTATATGGAATATTCCAAGTTGC
TACAATGAAAAAAGCGATCTTTCCGAAGCCTGGTTATATGCAATCATCTCTGTTATGGTGTTCTGCAGTACCATCTT
AGCCCTAGCTATATATCCCTACCTTGACATTGGCTGGAAACGAATAGATGCCATGAACCACCCAACTTTCCCCGCGC
CCGCTATGCTTCCACTGCAACAAGTTGTTGCCGGCGGCTTTGTCCCAGCCAATCAGCCTCGCCCCACTTCTCCCACC
CCCACTGAAATCAGCTACTTTAATCTAACAGGAGGAGATGACTGACACCCTAGATCTAGAAATGGACGGAATTATTA
CAGAGCAGCGCCTGCTAGAAAGACGCAGGGCAGCGGCCGAGCAACAGCGCATGAATCAAGAGCTCCAAGACATGGTT
AACTTGCACCAGTGCAAAAGGGGTATCTTTTGTCTGGTAAAGCAGGCCAAAGTCACCTACGACAGTAATACCACCGG
ACACCGCCTTAGCTACAAGTTGCCAACCAAGCGTCAGAAATTGGTGGTCATGGTGGGAGAAAAGCCCATTACCATAA
CTCAGCACTCGGTAGAAACCGAAGGCTGCATTCACTCACCTTGTCAAGGACCTGAGGATCTCTGCACCCTTATTAAG
ACCCTGTGCGGTCTCAAAGATCTTATTCCCTTTAACTAATAAAAAAAAATAATAAAGCATCACTTACTTAAAATCAG
TTAGCAAATTTCTGTCCAGTTTATTCAGCAGCACCTCCTTGCCCTCCTCCCAGCTCTGGTATTGCAGCTTCCTCCTG
GCTGCAAACTTTCTCCACAATCTAAATGGAATGTCAGTTTCCTCCTGTTCCTGTCCATCCGCACCCACTATCTTCAT
GTTGTTGCAGATGAAGCGCGCAAGACCGTCTGAAGATACCTTCAACCCCGTGTATCCATATGACACGGAAACCGGTC
CTCCAACTGTGCCTTTTCTTACTCCTCCCTTTGTATCCCCCAATGGGTTTCAAGAGAGTCCCCCTGGGGTACTCTCT
TTGCGCCTATCCGAACCTCTAGTTACCTCCAATGGCATGCTTGCGCTCAAAATGGGCAACGGCCTCTCTCTGGACGA
GGCCGGCAACCTTACCTCCCAAAATGTAACCACTGTGAGCCCACCTCTCAAAAAAACCAAGTCAAACATAAACCTGG
AAATATCTGCACCCCTCACAGTTACCTCAGAAGCCCTAACTGTGGCTGCCGCCGCACCTCTAATGGTCGCGGGCAAC
ACACTCACCATGCAATCACAGGCCCCGCTAACCGTGCACGACTCCAAACTTAGCATTGCCACCCAAGGACCCCTCAC
AGTGTCAGAAGGAAAGCTAGCCCTGCAAACATCAGGCCCCCTCACCACCACCGATAGCAGTACCCTTACTATCACTG
CCTCACCCCCTCTAACTACTGCCACTGGTAGCTTGGGCATTGACTTGAAAGAGCCCATTTATACACAAAATGGAAAA
CTAGGACTAAAGTACGGGGCTCCTTTGCATGTAACAGACGACCTAAACACTTTGACCGTAGCAACTGGTCCAGGTGT
GACTATTAATAATACTTCCTTGCAAACTAAAGTTACTGGAGCCTTGGGTTTTGATTCACAAGGCAATATGCAACTTA
ATGTAGCAGGAGGACTAAGGATTGATTCTCAAAACAGACGCCTTATACTTGATGTTAGTTATCCGTTTGATGCTCAA
AACCAACTAAATCTAAGACTAGGACAGGGCCCTCTTTTTATAAACTCAGCCCACAACTTGGATATTAACTACAACAA
AGGCCTTTACTTGTTTACAGCTTCAAACAATTCCAAAAAGCTTGAGGTTAACCTAAGCACTGCCAAGGGGTTGATGT
TTGACGCTACAGCCATAGCCATTAATGCAGGAGATGGGCTTGAATTTGGTTCACCTAATGCACCAAACACAAATCCC
CTCAAAACAAAAATTGGCCATGGCCTAGAATTTGATTCAAACAAGGCTATGGTTCCTAAACTAGGAACTGGCCTTAG
TTTTGACAGCACAGGTGCCATTACAGTAGGAAACAAAAATAATGATAAGCTAACTTTGTGGACCACACCAGCTCCAT
CTCCTAACTGTAGACTAAATGCAGAGAAAGATGCTAAACTCACTTTGGTCTTAACAAAATGTGGCAGTCAAATACTT
GCTACAGTTTCAGTTTTGGCTGTTAAAGGCAGTTTGGCTCCAATATCTGGAACAGTTCAAAGTGCTCATCTTATTAT
AAGATTTGACGAAAATGGAGTGCTACTAAACAATTCCTTCCTGGACCCAGAATATTGGAACTTTAGAAATGGAGATC
TTACTGAAGGCACAGCCTATACAAACGCTGTTGGATTTATGCCTAACCTATCAGCTTATCCAAAATCTCACGGTAAA
ACTGCCAAAAGTAACATTGTCAGTCAAGTTTACTTAAACGGAGACAAAACTAAACCTGTAACACTAACCATTACACT
AAACGGTACACAGGAAACAGGAGACACAACTCCAAGTGCATACTCTATGTCATTTTCATGGGACTGGTCTGGCCACA
ACTACATTAATGAAATATTTGCCACATCCTCTTACACTTTTTCATACATTGCCCAAGAATAAAGAATCGTTTGTGTT
ATGTTTCAACGTGTTTATTTTTCAATTGCAGAAAATTTCAAGTCATTTTTCATTCAGTAGTATAGCCCCACCACCAC
ATAGCTTATACAGATCACCGTACCTTAATCAAACTCACAGAACCCTAGTATTCAACCTGCCACCTCCCTCCCAACAC
ACAGAGTACACAGTCCTTTCTCCCCGGCTGGCCTTAAAAAGCATCATATCATGGGTAACAGACATATTCTTAGGTGT
TATATTCCACACGGTTTCCTGTCGAGCCAAACGCTCATCAGTGATATTAATAAACTCCCCGGGCAGCTCACTTAAGT
TCATGTCGCTGTCCAGCTGCTGAGCCACAGGCTGCTGTCCAACTTGCGGTTGCTTAACGGGCGGCGAAGGAGAAGTC
CACGCCTACATGGGGGTAGAGTCATAATCGTGCATCAGGATAGGGCGGTGGTGCTGCAGCAGCGCGCGAATAAACTG
CTGCCGCCGCCGCTCCGTCCTGCAGGAATACAACATGGCAGTGGTCTCCTCAGCGATGATTCGCACCGCCCGCAGCA
TAAGGCGCCTTGTCCTCCGGGCACAGCAGCGCACCCTGATCTCACTTAAATCAGCACAGTAACTGCAGCACAGCACC
ACAATATTGTTCAAAATCCCACAGTGCAAGGCGCTGTATCCAAAGCTCATGGCGGGGACCACAGAACCCACGTGGCC
ATCATACCACAAGCGCAGGTAGATTAAGTGGCGACCCCTCATAAACACGCTGGACATAAACATTACCTCTTTTGGCA
TGTTGTAATTCACCACCTCCCGGTACCATATAAACCTCTGATTAAACATGGCGCCATCCACCACCATCCTAAACCAG
CTGGCCAAAACCTGCCCGCCGGCTATACACTGCAGGGAACCGGGACTGGAACAATGACAGTGGAGAGCCCAGGACTC
GTAACCATGGATCATCATGCTCGTCATGATATCAATGTTGGCACAACACAGGCACACGTGCATACACTTCCTCAGGA
TTACAAGCTCCTCCCGCGTTAGAACCATATCCCAGGGAACAACCCATTCCTGAATCAGCGTAAATCCCACACTGCAG
GGAAGACCTCGCACGTAACTCACGTTGTGCATTGTCAAAGTGTTACATTCGGGCAGCAGCGGATGATCCTCCAGTAT
GGTAGCGCGGGTTTCTGTCTCAAAAGGAGGTAGACGATCCCTACTGTACGGAGTGCGCCGAGACAACCGAGATCGTG
TTGGTCGTAGTGTCATGCCAAATGGAACGCCGGACGTAGTCATATTTCCTGAAGCAAAACCAGGTGCGGGCGTGACA
AACAGATCTGCGTCTCCGGTCTCGCCGCTTAGATCGCTCTGTGTAGTAGTTGTAGTATATCCACTCTCTCAAAGCAT
CCAGGCGCCCCCTGGCTTCGGGTTCTATGTAAACTCCTTCATGCGCCGCTGCCCTGATAACATCCACCACCGCAGAA
TAAGCCACACCCAGCCAACCTACACATTCGTTCTGCGAGTCACACACGGGAGGAGCGGGAAGAGCTGGAAGAACCAT
GTTTTTTTTTTTATTCCAAAAGATTATCCAAAACCTCAAAATGAAGATCTATTAAGTGAACGCGCTCCCCTCCGGTG
GCGTGGTCAAACTCTACAGCCAAAGAACAGATAATGGCATTTGTAAGATGTTGCACAATGGCTTCCAAAAGGCAAAC
GGCCCTCACGTCCAAGTGGACGTAAAGGCTAAACCCTTCAGGGTGAATCTCCTCTATAAACATTCCAGCACCTTCAA
CCATGCCCAAATAATTCTCATCTCGCCACCTTCTCAATATATCTCTAAGCAAATCCCGAATATTAAGTCCGGCCATT
GTAAAAATCTGCTCCAGAGCGCCCTCCACCTTCAGCCTCAAGCAGCGAATCATGATTGCAAAAATTCAGGTTCCTCA
CAGACCTGTATAAGATTCAAAAGCGGAACATTAACAAAAATACCGCGATCCCGTAGGTCCCTTCGCAGGGCCAGCTG
AACATAATCGTGCAGGTCTGCACGGACCAGCGCGGCCACTTCCCCGCCAGGAACCATGACAAAAGAACCCACACTGA
TTATGACACGCATACTCGGAGCTATGCTAACCAGCGTAGCCCCGATGTAAGCTTGTTGCATGGGCGGCGATATAAAA
TGCAAGGTGCTGCTCAAAAAATCAGGCAAAGCCTCGCGCAAAAAAGAAAGCACATCGTAGTCATGCTCATGCAGATA
AAGGCAGGTAAGCTCCGGAACCACCACAGAAAAAGACACCATTTTTCTCTCAAACATGTCTGCGGGTTTCTGCATAA
ACACAAAATAAAATAACAAAAAAACATTTAAACATTAGAAGCCTGTCTTACAACAGGAAAAACAACCCTTATAAGCA
TAAGACGGACTACGGCCATGCCGGCGTGACCGTAAAAAAACTGGTCACCGTGATTAAAAAGCACCACCGACAGCTCC
TCGGTCATGTCCGGAGTCATAATGTAAGACTCGGTAAACACATCAGGTTGATTCACATCGGTCAGTGCTAAAAAGCG
ACCGAAATAGCCCGGGGGAATACATACCCGCAGGCGTAGAGACAACATTACAGCCCCCATAGGAGGTATAACAAAAT
TAATAGGAGAGAAAAACACATAAACACCTGAAAAACCCTCCTGCCTAGGCAAAATAGCACCCTCCCGCTCCAGAACA
ACATACAGCGCTTCCACAGCGGCAGCCATAACAGTCAGCCTTACCAGTAAAAAAGAAAACCTATTAAAAAAACACCA
CTCGACACGGCACCAGCTCAATCAGTCACAGTGTAAAAAAGGGCCAAGTGCAGAGCGAGTATATATAGGACTAAAAA
ATGACGTAACGGTTAAAGTCCACAAAAAACACCCAGAAAACCGCACGCGAACCTACGCCCAGAAACGAAAGCCAAAA
AACCCACAACTTCCTCAAATCGTCACTTCCGTTTTCCCACGTTACGTAACTTCCCATTTTAAGAAAACTACAATTCC
CAACACATACAAGTTACTCCGCCCTAAAACCTACGTCACCCGCCCCGTTCCCACGCCCCGCGCCACGTCACAAACTC
CACCCCCTCATTATCATATTGGCTTCAATCCAAAATAAGGTATATTATTGATGATG

GenBank Accession No. AP_000226 (SEQ ID NO: 241)

MKRARPSEDTFNPVYPYDTETGPPTVPFLTPPFVSPNGFQESPPGVLSLRLSEPLVTSNGMLALKMGNGLSLDEAGN
LTSQNVTTVSPPLKKTKSNINLEISAPLTVTSEALTVAAAAPLMVAGNTLTMQSQAPLTVHDSKLSIATQGPLTVSE
GKLALQTSGPLTTTDSSTLTITASPPLTTATGSLGIDLKEPIYTQNGKLGLKYGAPLHVTDDLNTLTVATGPGVTIN
NTSLQTKVTGALGFDSQGNMQLNVAGGLRIDSQNRRLILDVSYPFDAQNQLNLRLGQGPLFINSAHNLDINYNKGLY
LFTASNNSKKLEVNLSTAKGLMFDATAIAINAGDGLEFGSPNAPNINPLKTKIGHGLEFDSNKAMVPKLGTGLSFDS
TGAITVGNKNNDKLTLWTTPAPSPNCRLNAEKDAKLTLVLTKCGSQILATVSVLAVKGSLAPISGTVQSAHLIIRFD
ENGVLLNNSFLDPEYWNFRNGDLTEGTAYTNAVGFMPNLSAYPKSHGKTAKSNIVSQVYLNGDKTKPVTLTITLNGT
QETGDTTPSAYSMSFSWDWSGHNYINEIFATSSYTFSYIAQE

GenBank Accession No. AP_000206 (SEQ ID NO: 242)

MRRAAMYEEGPPPSYESVVSAAPVAAALGSPFDAPLDPPFVPPRYLRPTGGRNSIRYSELAPLFDTTRVYLVDNKST
DVASLNYQNDHSNFLTTVIQNNDYSPGEASTQTINLDDRSHWGGDLKTILHTNMPNVNEFMFTNKFKARVMVSRLPT
KDNQVELKYEWVEFTLPEGNYSETMTIDLMNNAIVEHYLKVGRQNGVLESDIGVKFDTRNFRLGFDPVTGLVMPGVY
TNEAFHPDIILLPGCGVDFTHSRLSNLLGIRKRQPFQEGFRITYDDLEGGNIPALLDVDAYQASLKDDTEQGGGGAG
GSNSSGSGAEENSNAAAAAMQPVEDMNDHAIRGDTFATRAEEKRAEAEAAAEAAAPAAQPEVEKPQKKPVIKPLTED
SKKRSYNLISNDSTFTQYRSWYLAYNYGDPQTGIRSWTLLCTPDVTCGSEQVYWSLPDMMQDPVTFRSTRQISNFPV
VGAELLPVHSKSFYNDQAVYSQLIRQFTSLTHVFNRFPENQILARPPAPTITTVSENVPALTDHGTLPLRNSIGGVQ
RVTITDARRRTCPYVYKALGIVSPRVLSSRTF

GenBank Accession No. AP_000211 (SEQ ID NO: 243)

MATPSMMPQWSYMHISGQDASEYLSPGLVQFARATETYFSLNNKFRNPTVAPTHDVTTDRSQRLTLRFIPVDREDTA
YSYKARFTLAVGDNRVLDMASTYFDIRGVLDRGPTFKPYSGTAYNALAPKGAPNPCEWDEAATALEINLEEEDDDNE
DEVDEQAEQQKTHVFGQAPYSGINITKEGIQIGVEGQTPKYADKTFQPEPQIGESQWYETEINHAAGRVLKKTTPMK
PCYGSYAKPTNENGGQGILVKQQNGKLESQVEMQFFSTTEATAGNGDNLTPKVVLYSEDVDIETPDTHISYMPTIKE
GNSRELMGQQSMPNRPNYIAFRDNFIGLMYYNSTGNMGVLAGQASQLNAVVDLQDRNTELSYQLLLDSIGDRTRYFS
MWNQAVDSYDPDVRIIENHGTEDELPNYCFPLGGVINTETLTKVKPKTGQENGWEKDATEFSDKNEIRVGNNFAMEI
NLNANLWRNFLYSNIALYLPDKLKYSPSNVKISDNPNTYDYMNKRVVAPGLVDCYINLGARWSLDYMDNVNPFNHHR
NAGLRYRSMLLGNGRYVPFHIQVPQKFFAIKNLLLLPGSYTYEWNFRKDVNMVLQSSLGNDLRVDGASIKFDSICLY
ATFFPMAHNTASTLEAMLRNDTNDQSFNDYLSAANMLYPIPANATNVPISIPSRNWAAFRGWAFTRLKTKETPSLGS
GYDPYYTYSGSIPYLDGTFYLNHTFKKVAITFDSSVSWPGNDRLLTPNEFEIKRSVDGEGYNVAQCNMTKDWFLVQM
LANYNIGYQGFYIPESYKDRMYSFFRNFQPMSRQVVDDTKYKDYQQVGILHQHNNSGFVGYLAPTMREGQAYPANFP
YPLIGKTAVDSITQKKFLCDRTLWRIPFSSNFMSMGALTDLGQNLLYANSAHALDMTFEVDPMDEPTLLYVLFEVFD
VVRVHRPHRGVIETVYLRTPFSAGNATT

GenBank Accession No. AY128640 (SEQ ID NO: 209)

CATCATCAATAATATACCTTATAGATGGAATGGTGCCAATATGTAAATGAGGTGATTTTAAAAAGTGTGGGCCGTGT
GGTGATTGGCTGTGGGGTTAACGGTTAAAAGGGGCGGCGCGGCCGTGGGAAAATGACGTTTTATGGGGGTGGAGTTT
TTTTGCAAGTTGTCGCGGGAAATGTTACGCATAAAAAGGCTTCTTTTCTCACGGAACTACTTAGTTTTCCCACGGTA
TTTAACAGGAAATGAGGTAGTTTTGACCGGATGCAAGTGAAAATTGCTGATTTTCGCGCGAAAACTGAATGAGGAAG
TGTTTTTCTGAATAATGTGGTATTTATGGCAGGGTGGAGTATTTGTTCAGGGCCAGGTAGACTTTGACCCATTACGT
GGAGGTTTCGATTACCGTGTTTTTTACCTGAATTTCCGCGTACCGTGTCAAAGTCTTCTGTTTTTACGTAGGTGTCA
GCTGATCGCTAGGGTATTTATACCTCAGGGTTTGTGTCAAGAGGCCACTCTTGAGTGCCAGCGAGAAGAGTTTTCTC
CTCTGCGCCGGCAGTTTAATAATAAAAAAATGAGAGATTTGCGATTTCTGCCTCAGGAAATAATCTCTGCTGAGACT
GGAAATGAAATATTGGAGCTTGTGGTGCACGCCCTGATGGGAGACGATCCGGAGCCACCTGTGCAGCTTTTTGAGCC
TCCTACGCTTCAGGAACTGTATGATTTAGAGGTAGAGGGATCGGAGGATTCTAATGAGGAAGCTGTGAATGGCTTTT
TTACCGATTCTATGCTTTTAGCTGCTAATGAAGGATTAGAATTAGATCCGCCTTTGGACACTTTCAATACTCCAGGG
GTGATTGTGGAAAGCGGTACAGGTGTAAGAAAATTACCTGATTTGAGTTCCGTGGACTGTGATTTGCACTGCTATGA
AGACGGGTTTCCTCCGAGTGATGAGGAGGACCATGAAAAGGAGCAGTCCATGCAGACTGCAGCGGGTGAGGGAGTGA
AGGCTGCCAATGTTGGTTTTCAGTTGGATTGCCCGGAGCTTCCTGGACATGGCTGTAAGTCTTGTGAATTTCACAGG
AAAAATACTGGAGTAAAGGAACTGTTATGTTCGCTTTGTTATATGAGAACGCACTGCCACTTTATTTACAGTAAGTG
TGTTTAAGTTAAAATTTAAAGGAATATGCTGTTTTTCACATGTATATTGAGTGTGAGTTTTGTGCTTCTTATTATAG
GTCCTGTGTCTGATGCTGATGAATCACCATCTCCTGATTCTACTACCTCACCTCCTGATATTCAAGCACCTGTTCCT
GTGGACGTGCGCAAGCCCATTCCTGTGAAGCTTAAGCCTGGGAAACGTCCAGCAGTGGAGAAACTTGAGGACTTGTT
ACAGGGTGGGGACGGACCTTTGGACTTGAGTACACGGAAACGTCCAAGACAATAAGTGTTCCATATCCGTGTTTACT
TAAGGTGACGTCAATATTTGTGTGAGAGTGCAATGTAATAAAAATATGTTAACTGTTCACTGGTTTTTATTGCTTTT
TGGGCGGGGACTCAGGTATATAAGTAGAAGCAGACCTGTGTGGTTAGCTCATAGGAGCTGGCTTTCATCCATGGAGG
TTTGGGCCATTTTGGAAGACCTTAGGAAGACTAGGCAACTGTTAGAGAGCGCTTCGGACGGAGTCTCCGGTTTTTGG
AGATTCTGGTTCGCTAGTGAATTAGCTAGGGTAGTTTTTAGGATAAAACAGGACTATAAACAAGAATTTGAAAAGTT
GTTGGTAGATTGCCCAGGACTTTTTGAAGCTCTTAATTTGGGCCATCAGGTTCACTTTAAAGAAAAAGTTTTATCAG
TTTTAGACTTTTCAACCCCAGGTAGAACTGCTGCTGCTGTGGCTTTTCTTACTTTTATATTAGATAAATGGATCCCG
CAGACTCATTTCAGCAGGGGATACGTTTTGGATTTCATAGCCACAGCATTGTGGAGAACATGGAAGGTTCGCAAGAT
GAGGACAATCTTAGGTTACTGGCCAGTGCAGCCTTTGGGTGTAGCGGGAATCCTGAGGCATCCACCGGTCATGCCAG
CGGTTCTGGAGGAGGAACAGCAAGAGGACAACCCGAGAGCCGGCCTGGACCCTCCAGTGGAGGAGGCGGAGTAGCTG
ACTTGTCTCCTGAACTGCAACGGGTGCTTACTGGATCTACGTCCACTGGACGGGATAGGGGCGTTAAGAGGGAGAGG
GCATCCAGTGGTACTGATGCTAGATCTGAGTTGGCTTTAAGTTTAATGAGTCGCAGACGTCCTGAAACCATTTGGTG
GCATGAGGTTCAGAAAGAGGGAAGGGATGAAGTTTCTGTATTGCAGGAGAAATATTCACTGGAACAGGTGAAAACAT
GTTGGTTGGAGCCAGAGGATGATTGGGAGGTGGCCATTAAAAATTATGCCAAGATAGCTTTGAGGCCTGATAAACAG
TATAAGATCAGTAGACGGATTAATATCCGGAATGCTTGTTACATATCTGGAAATGGGGCTGAGGTGGTAATAGATAC
TCAAGACAAGACAGTTATTAGATGCTGCATGATGGATATGTGGCCTGGAGTAGTCGGTATGGAAGCAGTCACTTTTG
TAAATGTTAAGTTTAGGGGAGATGGTTATAATGGAATAGTGTTTATGGCCAATACCAAACTTATATTGCATGGTTGT
AGCTTTTTTGGTTTCAACAATACCTGTGTAGATGCCTGGGGACAGGTTAGTGTACGGGGGTGTAGTTTCTATGCGTG
TTGGATTGCCACAGCTGGCAGAACCAAGAGTCAATTGTCTCTGAAGAAATGCATATTCCAAAGATGTAACCTGGGCA
TTCTGAATGAAGGCGAAGCAAGGGTCCGTCACTGCGCTTCTACAGATACTGGATGTTTTATTTTAATTAAGGGAAAT
GCCAGCGTAAAGCATAACATGATTTGTGGTGCTTCCGATGAGAGGCCTTATCAAATGCTCACTTGTGCTGGTGGGCA
TTGTAATATGCTGGCTACTGTGCATATTGTTTCCCATCAACGCAAAAAATGGCCTGTTTTTGATCACAATGTGTTGA
CCAAGTGCACCATGCATGCAGGTGGGCGTAGAGGAATGTTTATGCCTTACCAGTGTAACATGAATCATGTGAAAGTG
TTGTTGGAACCAGATGCCTTTTCCAGAATGAGCCTAACAGGAATCTTTGACATGAACACGCAAATCTGGAAGATCCT
GAGGTATGATGATACGAGATCGAGGGTGCGCGCATGCGAATGCGGAGGCAAGCATGCCAGGTTCCAGCCGGTGTGTG
TAGATGTGACCGAAGATCTCAGACCGGATCATTTGGTTATTGCCCGCACTGGAGCAGAGTTCGGATCCAGTGGAGAA
GAAACTGACTAAGGTGAGTATTGGGAAAACTTTGGGGTGGGATTTTCAGATGGACAGATTGAGTAAAAATTTGTTTT
TTCTGTCTTGCAGCTGACATGAGTGGAAATGCTTCTTTTAAGGGGGGAGTCTTCAGCCCTTATCTGACAGGGCGTCT
CCCATCCTGGGCAGGAGTTCGTCAGAATGTTATGGGATCTACTGTGGATGGAAGACCCGTTCAACCCGCCAATTCTT
CAACGCTGACCTATGCTACTTTAAGTTCTTCACCTTTGGACGCAGCTGCAGCCGCTGCCGCCGCCTCTGTCGCCGCT
AACACTGTGCTTGGAATGGGTTACTATGGAAGCATCGTGGCTAATTCCACTTCCTCTAATAACCCTTCTACACTGAC
TCAGGACAAGTTACTTGTCCTTTTGGCCCAGCTGGAGGCTTTGACCCAACGTCTGGGTGAACTTTCTCAGCAGGTGG
CCGAGTTGCGAGTACAAACTGAGTCTGCTGTCGGCACGGCAAAGTCTAAATAAAAAAAATTCCAGAATCAATGAATA
AATAAACGAGCTTGTTGTTGATTTAAAATCAAGTGTTTTTATTTCATTTTTCGCGCACGGTATGCCCTGGACCACCG
ATCTCGATCATTGAGAACTCGGTGGATTTTTTCCAGAATCCTATAGAGGTGGGATTGAATGTTTAGATACATGGGCA
TTAGGCCGTCTTTGGGGTGGAGATAGCTCCATTGAAGGGATTCATGCTCCGGGGTAGTGTTGTAAATCACCCAGTCA
TAACAAGGTCGCAGTGCATGGTGTTGCACAATATCTTTTAGAAGTAGGCTGATTGCCACAGATAAGCCCTTGGTGTA
GGTGTTTACAAACCGGTTGAGCTGGGAGGGGTGCATTCGAGGTGAAATTATGTGCATTTTGGATTGGATTTTTAAGT
TGGCAATATTGCCGCCAAGATCCCGTCTTGGGTTCATGTTATGAAGGACTACCAAGACGGTGTATCCGGTACATTTA
GGAAATTTATCGTGCAGCTTGGATGGAAAAGCGTGGAAAAATTTGGAGACACCCTTGTGTCCTCCGAGATTTTCCAT
GCACTCATCCATGATAATAGCAATGGGGCCGTGGGCAGCGGCGCGGGCAAACACGTTCCGTGGGTCTGACACATCAT
AGTTATGTTCCTGAGTTAAATCATCATAAGCCATTTTAATGAATTTGGGGCGGAGCGTACCAGATTGGGGTATGAAT
GTTCCTTCGGGCCCCGGAGCATAGTTCCCCTCACAGATTTGCATTTCCCAAGCTTTCAGTTCTGAGGGTGGAATCAT
GTCCACCTGGGGGGCTATGAAGAACACCGTTTCGGGGGCGGGGGTGATTAGTTGGGATGATAGCAAGTTTCTGAGCA
ATTGAGATTTGCCACATCCGGTGGGGCCATAAATAATTCCGATTACAGGTTGCAGGTGGTAGTTTAGGGAACGGCAA
CTGCCGTCTTCTCGAAGCAAGGGGGCCACCTCGTTCATCATTTCCCTTACATGCATATTTTCCCGCACCAAATCCAT
TAGGAGGCGCTCTCCTCCTAGTGATAGAAGTTCTTGTAGTGAGGAAAAGTTTTTCAGCGGTTTTAGACCGTCAGCCA
TGGGCATTTTGGAAAGAGTTTGCTGCAAAAGTTCTAGTCTGTTCCACAGTTCAGTGATGTGTTCTATGGCATCTCGA
TCCAGCAGACCTCCTCGTTTCGCGGGTTTGGACGGCTCCTGGAGTAGGGTATGAGACGATGGGCGTCCAGCGCTGCC
AGGGTTCGGTCCTTCCAGGGTCTCAGTGTTCGAGTCAGGGTTGTTTCCGTCACAGTGAAGGGGTGTGCGCCTGCTTG
GGCGCTTGCCAGGGTGCGCTTCAGACTCATTCTGCTGGTGGAGAACTTCTGTCGCTTGGCGCCCTGTATGTCGGCCA
AGTAGCAGTTTACCATGAGTTCGTAGTTGAGCGCCTCGGCTGCGTGGCCTTTGGCGCGGAGCTTACCTTTGGAAGTT
TTCTTGCATACCGGGCAGTATAGGCATTTCAGCGCATACAGCTTGGGCGCAAGGAAAATGGATTCTGGGGAGTATGC
ATCCGCGCCGCAGGAGGCGCAAACAGTTTCACATTCCACCAGCCAGGTTAAATCCGGTTCATTGGGGTCAAAAACAA
GTTTTCCGCCATATTTTTTGATGCGTTTCTTACCTTTGGTCTCCATAAGTTCGTGTCCTCGTTGAGTGACAAACAGG
CTGTCCGTATCTCCGTAGACTGATTTTACAGGCCTCTTCTCCAGTGGAGTGCCTCGGTCTTCTTCGTACAGGAACTC
TGACCACTCTGATACAAAGGCGCGCGTCCAGGCCAGCACAAAGGAGGCTATGTGGGAGGGGTAGCGATCGTTGTCAA
CCAGGGGGTCCACCTTTTCCAAAGTATGCAAACACATGTCACCCTCTTCAACATCCAGGAATGTGATTGGCTTGTAG
GTGTATTTCACGTGACCTGGGGTCCCCGCTGGGGGGGTATAAAAGGGGGCGGTTCTTTGCTCTTCCTCACTGTCTTC
CGGATCGCTGTCCAGGAACGTCAGCTGTTGGGGTAGGTATTCCCTCTCGAAGGCGGGCATGACCTCTGCACTCAGGT
TGTCAGTTTCTAAGAACGAGGAGGATTTGATATTGACAGTGCCGGTTGAGATGCCTTTCATGAGGTTTTCGTCCATT
TGGTCAGAAAACACAATTTTTTTATTGTCAAGTTTGGTGGCAAATGATCCATACAGGGCGTTGGATAAAAGTTTGGC
AATGGATCGCATGGTTTGGTTCTTTTCCTTGTCCGCGCGCTCTTTGGCGGCGATGTTGAGTTGGACATACTCGCGTG
CCAGGCACTTCCATTCGGGGAAGATAGTTGTTAATTCATCTGGCACGATTCTCACTTGCCACCCTCGATTATGCAAG
GTAATTAAATCCACACTGGTGGCCACCTCGCCTCGAAGGGGTTCATTGGTCCAACAGAGCCTACCTCCTTTCCTAGA
ACAGAAAGGGGGAAGTGGGTCTAGCATAAGTTCATCGGGAGGGTCTGCATCCATGGTAAAGATTCCCGGAAGTAAAT
CCTTATCAAAATAGCTGATGGGAGTGGGGTCATCTAAGGCCATTTGCCATTCTCGAGCTGCCAGTGCGCGCTCATAT
GGGTTAAGGGGACTGCCCCAGGGCATGGGATGGGTGAGAGCAGAGGCATACATGCCACAGATGTCATAGACGTAGAT
GGGATCCTCAAAGATGCCTATGTAGGTTGGATAGCATCGCCCCCCTCTGATACTTGCTCGCACATAGTCATATAGTT
CATGTGATGGCGCTAGCAGCCCCGGACCCAAGTTGGTGCGATTGGGTTTTTCTGTTCTGTAGACGATCTGGCGAAAG
ATGGCGTGAGAATTGGAAGAGATGGTGGGTCTTTGAAAAATGTTGAAATGGGCATGAGGTAGACCTACAGAGTCTCT
GACAAAGTGGGCATAAGATTCTTGAAGCTTGGTTACCAGTTCGGCGGTGACAAGTACGTCTAGGGCGCAGTAGTCAA
GTGTTTCTTGAATGATGTCATAACCTGGTTGGTTTTTCTTTTCCCACAGTTCGCGGTTGAGAAGGTATTCTTCGCGA
TCCTTCCAGTACTCTTCTAGCGGAAACCCGTCTTTGTCTGCACGGTAAGATCCTAGCATGTAGAACTGATTAACTGC
CTTGTAAGGGCAGCAGCCCTTCTCTACGGGTAGAGAGTATGCTTGAGCAGCTTTTCGTAGCGAAGCGTGAGTAAGGG
CAAAGGTGTCTCTGACCATGACTTTGAGAAATTGGTATTTGAAGTCCATGTCGTCACAGGCTCCCTGTTCCCAGAGT
TGGAAGTCTACCCGTTTCTTGTAGGCGGGGTTGGGCAAAGCGAAAGTAACATCATTGAAGAGAATCTTACCGGCTCT
GGGCATAAAATTGCGAGTGATGCGGAAAGGCTGTGGTACTTCCGCTCGATTGTTGATCACCTGGGCAGCTAGGACGA
TTTCGTCGAAACCGTTGATGTTGTGTCCTACGATGTATAATTCTATGAAACGCGGCGTGCCTCTGACGTGAGGTAGC
TTACTGAGCTCATCAAAGGTTAGGTCTGTGGGGTCAGATAAGGCGTAGTGTTCGAGAGCCCATTCGTGCAGGTGAGG
ATTTGCATGTAGGAATGATGACCAAAGATCTACCGCCAGTGCTGTTTGTAACTGGTCCCGATACTGACGAAAATGCC
GGCCAATTGCCATTTTTTCTGGAGTGACACAGTAGAAGGTTCTGGGGTCTTGTTGCCATCGATCCCACTTGAGTTTA
ATGGCTAGATCGTGGGCCATGTTGACGAGACGCTCTTCTCCTGAGAGTTTCATGACCAGCATGAAAGGAACTAGTTG
TTTGCCAAAGGATCCCATCCAGGTGTAAGTTTCCACATCGTAGGTCAGGAAGAGTCTTTCTGTGCGAGGATGAGAGC
CGATCGGGAAGAACTGGATTTCCTGCCACCAGTTGGAGGATTGGCTGTTGATGTGATGGAAGTAGAAGTTTCTGCGG
CGCGCCGAGCATTCGTGTTTGTGCTTGTACAGACGGCCGCAGTAGTCGCAGCGTTGCACGGGTTGTATCTCGTGAAT
GAGCTGTACCTGGCTTCCCTTGACGAGAAATTTCAGTGGGAAGCCGAGGCCTGGCGATTGTATCTCGTGCTCTTCTA
TATTCGCTGTATCGGCCTGTTCATCTTCTGTTTCGATGGTGGTCATGCTGACGAGCCCCCGCGGGAGGCAAGTCCAG
ACCTCGGCGCGGGAGGGGCGGAGCTGAAGGACGAGAGCGCGCAGGCTGGAGCTGTCCAGAGTCCTGAGACGCTGCGG
ACTCAGGTTAGTAGGTAGGGACAGAAGATTAACTTGCATGATCTTTTCCAGGGCGTGCGGGAGGTTCAGATGGTACT
TGATTTCCACAGGTTCGTTTGTAGAGACGTCAATGGCTTGCAGGGTTCCGTGTCCTTTGGGCGCCACTACCGTACCT
TTGTTTTTTCTTTTGATCGGTGGTGGCTCTCTTGCTTCTTGCATGCTCAGAAGCGGTGACGGGGACGCGCGCCGGGC
GGCAGCGGTTGTTCCGGACCCGGGGGCATGGCTGGTAGTGGCACGTCGGCGCCGCGCACGGGCAGGTTCTGGTATTG
CGCTCTGAGAAGACTTGCGTGCGCCACCACGCGTCGATTGACGTCTTGTATCTGACGTCTCTGGGTGAAAGCTACCG
GCCCCGTGAGCTTGAACCTGAAAGAGAGTTCAACAGAATCAATTTCGGTATCGTTAACGGCAGCTTGTCTCAGTATT
TCTTGTACGTCACCAGAGTTGTCCTGGTAGGCGATCTCCGCCATGAACTGCTCGATTTCTTCCTCCTGAAGATCTCC
GCGACCCGCTCTTTCGACGGTGGCCGCGAGGTCATTGGAGATACGGCCCATGAGTTGGGAGAATGCATTCATGCCCG
CCTCGTTCCAGACGCGGCTGTAAACCACGGCCCCCTCGGAGTCTCTTGCGCGCATCACCACCTGAGCGAGGTTAAGC
TCCACGTGTCTGGTTAAGACCGCATAGTTGCATAGGCGCTGAAAAAGGTAGTTGAGTGTGGTGGCAATGTGTTCGGC
GACGAAGAAATACATGATCCATCGTCTCAGCGGCATTTCGCTAACATCGCCCAGAGCTTCCAAGCGCTCCATGGCCT
CGTAGAAGTCCACGGCAAAATTAAAAAACTGGGAGTTTCGCGCGGACACGGTCAATTCCTCCTCGAGAAGACGGATG
AGTTCGGCTATGGTGGCCCGTACTTCGCGTTCGAAGGCTCCCGGGATCTCTTCTTCCTCTTCTATCTCTTCTTCCAC
TAACATCTCTTCTTCGTCTTCAGGCGGGGGCGGAGGGGGCACGCGGCGACGTCGACGGCGCACGGGCAAACGGTCGA
TGAATCGTTCAATGACCTCTCCGCGGCGGCGGCGCATGGTTTCAGTGACGGCGCGGCCGTTCTCGCGCGGTCGCAGA
GTAAAAACACCGCCGCGCATCTCCTTAAAGTGGTGACTGGGAGGTTCTCCGTTTGGGAGGGAGAGGGCGCTGATTAT
ACATTTTATTAATTGGCCCGTAGGGACTGCGCGCAGAGATCTGATCGTGTCAAGATCCACGGGATCTGAAAACCTTT
CGACGAAAGCGTCTAACCAGTCACAGTCACAAGGTAGGCTGAGTACGGCTTCTTGTGGGCGGGGGTGGTTATGTGTT
CGGTCTGGGTCTTCTGTTTCTTCTTCATCTCGGGAAGGTGAGACGATGCTGCTGGTGATGAAATTAAAGTAGGCAGT
TCTAAGACGGCGGATGGTGGCGAGGAGCACCAGGTCTTTGGGTCCGGCTTGCTGGATACGCAGGCGATTGGCCATTC
CCCAAGCATTATCCTGACATCTAGCAAGATCTTTGTAGTAGTCTTGCATGAGCCGTTCTACGGGCACTTCTTCCTCA
CCCGTTCTGCCATGCATACGTGTGAGTCCAAATCCGCGCATTGGTTGTACCAGTGCCAAGTCAGCTACGACTCTTTC
GGCGAGGATGGCTTGCTGTACTTGGGTAAGGGTGGCTTGAAAGTCATCAAAATCCACAAAGCGGTGGTAAGCCCCTG
TATTAATGGTGTAAGCACAGTTGGCCATGACTGACCAGTTAACTGTCTGGTGACCAGGGCGCACGAGCTCGGTGTAT
TTAAGGCGCGAATAGGCGCGGGTGTCAAAGATGTAATCGTTGCAGGTGCGCACCAGATACTGGTACCCTATAAGAAA
ATGCGGCGGTGGTTGGCGGTAGAGAGGCCATCGTTCTGTAGCTGGAGCGCCAGGGGCGAGGTCTTCCAACATAAGGC
GGTGATAGCCGTAGATGTACCTGGACATCCAGGTGATTCCTGCGGCGGTAGTAGAAGCCCGAGGAAACTCGCGTACG
CGGTTCCAAATGTTGCGTAGCGGCATGAAGTAGTTCATTGTAGGCACGGTTTGACCAGTGAGGCGCGCGCAGTCATT
GATGCTCTATAGACACGGAGAAAATGAAAGCGTTCAGCGACTCGACTCCGTAGCCTGGAGGAACGTGAACGGGTTGG
GTCGCGGTGTACCCCGGTTCGAGACTTGTACTCGAGCCGGCCGGAGCCGCGGCTAACGTGGTATTGGCACTCCCGTC
TCGACCCAGCCTACAAAAATCCAGGATACGGAATCGAGTCGTTTTGCTGGTTTCCGAATGGCAGGGAAGTGAGTCCT
ATTTTTTTTTTTTTTTGCCGCTCAGAATGCATCCCGTGCTGCGACAGATGCGCCCCCAACAACAGCCCCCCTCGCAG
CAGCAGCAGCAGCAACCACAAAAGGCTGTCCCTGCAACTACTGCAACTGCCGCCGTGAGCGGTGCGGGACAGCCCGC
CTATGATCTGGACTTGGAAGAGGGCGAAGGACTGGCACGTCTAGGTGCGCCTTCGCCCGAGCGGCATCCGCGAGTTC
AACTGAAAAAAGATTCTCGCGAGGCGTATGTGCCCCAACAGAACCTATTTAGAGACAGAAGCGGCGAGGAGCCGGAG
GAGATGCGAGCTTCCCGCTTTAACGCGGGTCGTGAGCTGCGTCACGGTTTGGACCGAAGACGAGTGTTGCGAGACGA
GGATTTCGAAGTTGATGAAGTGACAGGGATCAGTCCTGCCAGGGCACACGTGGCTGCAGCCAACCTTGTATCGGCTT
ACGAGCAGACAGTAAAGGAAGAGCGTAACTTCCAAAAGTCTTTTAATAATCATGTGCGAACCCTGATTGCCCGCGAA
GAAGTTACCCTTGGTTTGATGCATTTGTGGGATTTGATGGAAGCTATCATTCAGAACCCTACTAGCAAACCTCTGAC
CGCCCAGCTGTTTCTGGTGGTGCAACACAGCAGAGACAATGAGGCTTTCAGAGAGGCGCTGCTGAACATCACCGAAC
CCGAGGGGAGATGGTTGTATGATCTTATCAACATTCTACAGAGTATCATAGTGCAGGAGCGGAGCCTGGGCCTGGCC
GAGAAGGTAGCTGCCATCAATTACTCGGTTTTGAGCTTGGGAAAATATTACGCTCGCAAAATCTACAAGACTCCATA
CGTTCCCATAGACAAGGAGGTGAAGATAGATGGGTTCTACATGCGCATGACGCTCAAGGTCTTGACCCTGAGCGATG
ATCTTGGGGTGTATCGCAATGACAGAATGCATCGCGCGGTTAGCGCCAGCAGGAGGCGCGAGTTAAGCGACAGGGAA
CTGATGCACAGTTTGCAAAGAGCTCTGACTGGAGCTGGAACCGAGGGTGAGAATTACTTCGACATGGGAGCTGACTT
GCAGTGGCAGCCTAATCGCAGGGCTCTGAGCGCCGCGACGGCAGGATGTGAGCTTCCTTACATAGAAGAGGCGGATG
AAGGCGAGGAGGAAGAGGGCGAGTACTTGGAAGACTGATGGCACAACCCGTGTTTTTTGCTAGATGGAACAGCAAGC
ACCGGATCCCGCAATGCGGGCGGCGCTGCAGAGCCAGCCGTCCGGCATTAACTCCTCGGACGATTGGACCCAGGCCA
TGCAACGTATCATGGCGTTGACGACTCGCAACCCCGAAGCCTTTAGACAGCAACCCCAGGCCAACCGTCTATCGGCC
ATCATGGAAGCTGTAGTGCCTTCCCGATCTAATCCCACTCATGAGAAGGTCCTGGCCATCGTGAACGCGTTGGTGGA
GAACAAAGCTATTCGTCCAGATGAGGCCGGACTGGTATACAACGCTCTCTTAGAACGCGTGGCTCGCTACAACAGTA
GCAATGTGCAAACCAATTTGGACCGTATGATAACAGATGTACGCGAAGCCGTGTCTCAGCGCGAAAGGTTCCAGCGT
GATGCCAACCTGGGTTCGCTGGTGGCGTTAAATGCTTTCTTGAGTACTCAGCCTGCTAATGTGCCGCGTGGTCAACA
GGATTATACTAACTTTTTAAGTGCTTTGAGACTGATGGTATCAGAAGTACCTCAGAGCGAAGTGTATCAGTCCGGTC
CTGATTACTTCTTTCAGACTAGCAGACAGGGCTTGCAGACGGTAAATCTGAGCCAAGCTTTTAAAAACCTTAAAGGT
TTGTGGGGAGTGCATGCCCCGGTAGGAGAAAGAGCAACCGTGTCTAGCTTGTTAACTCCGAACTCCCGCCTGTTATT
ACTGTTGGTAGCTCCTTTCACCGACAGCGGTAGCATCGACCGTAATTCCTATTTGGGTTACCTACTAAACCTGTATC
GCGAAGCCATAGGGCAAAGTCAGGTGGACGAGCAGACCTATCAAGAAATTACCCAAGTCAGTCGCGCTTTGGGACAG
GAAGACACTGGCAGTTTGGAAGCCACTCTGAACTTCTTGCTTACCAATCGGTCTCAAAAGATCCCTCCTCAATATGC
TCTTACTGCGGAGGAGGAGAGGATCCTTAGATATGTGCAGCAGAGCGTGGGATTGTTTCTGATGCAAGAGGGGGCAA
CTCCGACTGCAGCACTGGACATGACAGCGCGAAATATGGAGCCCAGCATGTATGCCAGTAACCGACCTTTCATTAAC
AAACTGCTGGACTACTTGCACAGAGCTGCCGCTATGAACTCTGATTATTTCACCAATGCCATCTTAAACCCGCACTG
GCTGCCCCCACCTGGTTTCTACACGGGCGAATATGACATGCCCGACCCTAATGACGGATTTCTGTGGGACGACGTGG
ACAGCGATGTTTTTTCACCTCTTTCTGATCATCGCACGTGGAAAAAGGAAGGCGGTGATAGAATGCATTCTTCTGCA
TCGCTGTCCGGGGTCATGGGTGCTACCGCGGCTGAGCCCGAGTCTGCAAGTCCTTTTCCTAGTCTACCCTTTTCTCT
ACACAGTGTACGTAGCAGCGAAGTGGGTAGAATAAGTCGCCCGAGTTTAATGGGCGAAGAGGAGTACCTAAACGATT
CCTTGCTCAGACCGGCAAGAGAAAAAAATTTCCCAAACAATGGAATAGAAAGTTTGGTGGATAAAATGAGTAGATGG
AAGACTTATGCTCAGGATCACAGAGACGAGCCTGGGATCATGGGGACTACAAGTAGAGCGAGCCGTAGACGCCAGCG
CCATGACAGACAGAGGGGTCTTGTGTGGGACGATGAGGATTCGGCCGATGATAGCAGCGTGTTGGACTTGGGTGGGA
GAGGAAGGGGCAACCCGTTTGCTCATTTGCGCCCTCGCTTGGGTGGTATGTTGTGAAAAAAAATAAAAAAGAAAAAC
TCACCAAGGCCATGGCGACGAGCGTACGTTCGTTCTTCTTTATTATCTGTGTCTAGTATAATGAGGCGAGTCGTGCT
AGGCGGAGCGGTGGTGTATCCGGAGGGTCCTCCTCCTTCGTACGAGAGCGTGATGCAGCAGCAGCAGGCGACGGCGG
TGATGCAATCCCCACTGGAGGCTCCCTTTGTGCCTCCGCGATACCTGGCACCTACGGAGGGCAGAAACAGCATTCGT
TACTCGGAACTGGCACCTCAGTACGATACCACCAGGTTGTATCTGGTGGACAACAAGTCGGCGGACATTGCTTCTCT
GAACTATCAGAATGACCACAGCAACTTCTTGACCACGGTGGTGCAGAACAATGACTTTACCCCTACGGAAGCCAGCA
CCCAGACCATTAACTTTGATGAACGATCGCGGTGGGGCGGTCAGCTAAAGACCATCATGCATACTAACATGCCAAAC
GTGAACGAGTATATGTTTAGTAACAAGTTCAAAGCGCGTGTGATGGTGTCCAGAAAACCTCCCGACGGTGCTGCAGT
TGGGGATACTTATGATCACAAGCAGGATATTTTGGAATATGAGTGGTTCGAGTTTACTTTGCCAGAAGGCAACTTTT
CAGTTACTATGACTATTGATTTGATGAACAATGCCATCATAGATAATTACTTGAAAGTGGGTAGACAGAATGGAGTG
CTTGAAAGTGACATTGGTGTTAAGTTCGACACCAGGAACTTCAAGCTGGGATGGGATCCCGAAACCAAGTTGATCAT
GCCTGGAGTGTATACGTATGAAGCCTTCCATCCTGACATTGTCTTACTGCCTGGCTGCGGAGTGGATTTTACCGAGA
GTCGTTTGAGCAACCTTCTTGGTATCAGAAAAAAACAGCCATTTCAAGAGGGTTTTAAGATTTTGTATGAAGATTTA
GAAGGTGGTAATATTCCGGCCCTCTTGGATGTAGATGCCTATGAGAACAGTAAGAAAGAACAAAAAGCCAAAATAGA
AGCTGCTACAGCTGCTGCAGAAGCTAAGGCAAACATAGTTGCCAGCGACTCTACAAGGGTTGCTAACGCTGGAGAGG
TCAGAGGAGACAATTTTGCGCCAACACCTGTTCCGACTGCAGAATCATTATTGGCCGATGTGTCTGAAGGAACGGAC
GTGAAACTCACTATTCAACCTGTAGAAAAAGATAGTAAGAATAGAAGCTATAATGTGTTGGAAGACAAAATCAACAC
AGCCTATCGCAGTTGGTATCTTTCGTACAATTATGGCGATCCCGAAAAAGGAGTGCGTTCCTGGACATTGCTCACCA
CCTCAGATGTCACCTGCGGAGCAGAGCAGGTTTACTGGTCGCTTCCAGACATGATGAAGGATCCTGTCACTTTCCGC
TCCACTAGACAAGTCAGTAACTACCCTGTGGTGGGTGCAGAGCTTATGCCCGTCTTCTCAAAGAGCTTCTACAACGA
ACAAGCTGTGTACTCCCAGCAGCTCCGCCAGTCCACCTCGCTTACGCACGTCTTCAACCGCTTTCCTGAGAACCAGA
TTTTAATCCGTCCGCCGGCGCCCACCATTACCACCGTCAGTGAAAACGTTCCTGCTCTCACAGATCACGGGACCCTG
CCGTTGCGCAGCAGTATCCGGGGAGTCCAACGTGTGACCGTTACTGACGCCAGACGCCGCACCTGTCCCTACGTGTA
CAAGGCACTGGGCATAGTCGCACCGCGCGTCCTTTCAAGCCGCACTTTCTAAAAAAAAAATGTCCATTCTTATCTCG
CCCAGTAATAACACCGGTTGGGGTCTGCGCGCTCCAAGCAAGATGTACGGAGGCGCACGCAAACGTTCTACCCAACA
TCCCGTGCGTGTTCGCGGACATTTTCGCGCTCCATGGGGTGCCCTCAAGGGCCGCACTCGCGTTCGAACCACCGTCG
ATGATGTAATCGATCAGGTGGTTGCCGACGCCCGTAATTATACTCCTACTGCGCCTACATCTACTGTGGATGCAGTT
ATTGACAGTGTAGTGGCTGACGCTCGCAACTATGCTCGACGTAAGAGCCGGCGAAGGCGCATTGCCAGACGCCACCG
AGCTACCACTGCCATGCGAGCCGCAAGAGCTCTGCTACGAAGAGCTAGACGCGTGGGGCGAAGAGCCATGCTTAGGG
CGGCCAGACGTGCAGCTTCGGGCGCCAGCGCCGGCAGGTCCCGCAGGCAAGCAGCCGCTGTCGCAGCGGCGACTATT
GCCGACATGGCCCAATCGCGAAGAGGCAATGTATACTGGGTGCGTGACGCTGCCACCGGTCAACGTGTACCCGTGCG
CACCCGTCCCCCTCGCACTTAGAAGATACTGAGCAGTCTCCGATGTTGTGTCCCAGCGGCGAGGATGTCCAAGCGCA
AATACAAGGAAGAAATGCTGCAGGTTATCGCACCTGAAGTCTACGGCCAACCGTTGAAGGATGAAAAAAAACCCCGC
AAAATCAAGCGGGTTAAAAAGGACAAAAAAGAAGAGGAAGATGGCGATGATGGGCTGGCGGAGTTTGTGCGCGAGTT
TGCCCCACGGCGACGCGTGCAATGGCGTGGGCGCAAAGTTCGACATGTGTTGAGACCTGGAACTTCGGTGGTCTTTA
CACCCGGCGAGCGTTCAAGCGCTACTTTTAAGCGTTCCTATGATGAGGTGTACGGGGATGATGATATTCTTGAGCAG
GCGGCTGACCGATTAGGCGAGTTTGCTTATGGCAAGCGTAGTAGAATAACTTCCAAGGATGAGACAGTGTCAATACC
CTTGGATCATGGAAATCCCACCCCTAGTCTTAAACCGGTCACTTTGCAGCAAGTGTTACCCGTAACTCCGCGAACAG
GTGTTAAACGCGAAGGTGAAGATTTGTATCCCACTATGCAACTGATGGTACCCAAACGCCAGAAGTTGGAGGACGTT
TTGGAGAAAGTAAAAGTGGATCCAGATATTCAACCTGAGGTTAAAGTGAGACCCATTAAGCAGGTAGCGCCTGGTCT
GGGGGTACAAACTGTAGACATTAAGATTCCCACTGAAAGTATGGAAGTGCAAACTGAACCCGCAAAGCCTACTGCCA
CCTCCACTGAAGTGCAAACGGATCCATGGATGCCCATGCCTATTACAACTGACGCCGCCGGTCCCACTCGAAGATCC
CGACGAAAGTACGGTCCAGCAAGTCTGTTGATGCCCAATTATGTTGTACACCCATCTATTATTCCTACTCCTGGTTA
CCGAGGCACTCGCTACTATCGCAGCCGAAACAGTACCTCCCGCCGTCGCCGCAAGACACCTGCAAATCGCAGTCGTC
GCCGTAGACGCACAAGCAAACCGACTCCCGGCGCCCTGGTGCGGCAAGTGTACCGCAATGGTAGTGCGGAACCTTTG
ACACTGCCGCGTGCGCGTTACCATCCGAGTATCATCACTTAATCAATGTTGCCGCTGCCTCCTTGCAGATATGGCCC
TCACTTGTCGCCTTCGCGTTCCCATCACTGGTTACCGAGGAAGAAACTCGCGCCGTAGAAGAGGGATGTTGGGACGC
GGAATGCGACGCTACAGGCGACGGCGTGCTATCCGCAAGCAATTGCGGGGTGGTTTTTTACCAGCCTTAATTCCAAT
TATCGCTGCTGCAATTGGCGCGATACCAGGCATAGCTTCCGTGGCGGTTCAGGCCTCGCAACGACATTGACATTGGA
AAAAAAACGTATAAATAAAAAAAAATACAATGGACTCTGACACTCCTGGTCCTGTGACTATGTTTTCTTAGAGATGG
AAGACATCAATTTTTCATCCTTGGCTCCGCGACACGGCACGAAGCCGTACATGGGCACCTGGAGCGACATCGGCACG
AGCCAACTGAACGGGGGCGCCTTCAATTGGAGCAGTATCTGGAGCGGGCTTAAAAATTTTGGCTCAACCATAAAAAC
ATACGGGAACAAAGCTTGGAACAGCAGTACAGGACAGGCGCTTAGAAATAAACTTAAAGACCAGAACTTCCAACAAA
AAGTAGTCGATGGGATAGCTTCCGGCATCAATGGAGTGGTAGATTTGGCTAACCAGGCTGTGCAGAAAAAGATAAAC
AGTCGTTTGGACCCGCCGCCAGCAACCCCAGGTGAAATGCAAGTGGAGGAAGAAATTCCTCCGCCAGAAAAACGAGG
CGACAAGCGTCCGCGTCCCGATTTGGAAGAGACGCTGGTGACGCGCGTAGATGAACCGCCTTCTTATGAGGAAGCAA
CGAAGCTTGGAATGCCCACCACTAGACCGATAGCCCCAATGGCCACCGGGGTGATGAAACCTTCTCAGTTGCATCGA
CCCGTCACCTTGGATTTGCCCCCTCCCCCTGCTGCTACTGCTGTACCCGCTTCTAAGCCTGTCGCTGCCCCGAAACC
AGTCGCCGTAGCCAGGTCACGTCCCGGGGGCGCTCCTCGTCCAAATGCGCACTGGCAAAATACTCTGAACAGCATCG
TGGGTCTAGGCGTGCAAAGTGTAAAACGCCGTCGCTGCTTTTAATTAAATATGGAGTAGCGCTTAACTTGCCTATCT
GTGTATATGTGTCATTACACGCCGTCACAGCAGCAGAGGAAAAAAGGAAGAGGTCGTGCGTCGACGCTGAGTTACTT
TCAAGATGGCCACCCCATCGATGCTGCCCCAATGGGCATACATGCACATCGCCGGACAGGATGCTTCGGAGTACCTG
AGTCCGGGTCTGGTGCAGTTCGCCCGCGCCACAGACACCTACTTCAATCTGGGAAATAAGTTTAGAAATCCCACCGT
AGCGCCGACCCACGATGTGACCACCGACCGTAGCCAGCGGCTCATGTTGCGCTTCGTGCCCGTTGACCGGGAGGACA
ATACATACTCTTACAAAGTGCGGTACACCCTGGCCGTGGGCGACAACAGAGTGCTGGATATGGCCAGCACGTTCTTT
GACATTAGGGGCGTGTTGGACAGAGGTCCCAGTTTCAAACCCTATTCTGGTACGGCTTACAACTCTCTGGCTCCTAA
AGGCGCTCCAAATGCATCTCAATGGATTGCAAAAGGCGTACCAACTGCAGCAGCCGCAGGCAATGGTGAAGAAGAAC
ATGAAACAGAGGAGAAAACTGCTACTTACACTTTTGCCAATGCTCCTGTAAAAGCCGAGGCTCAAATTACAAAAGAG
GGCTTACCAATAGGTTTGGAGATTTCAGCTGAAAACGAATCTAAACCCATCTATGCAGATAAACTTTATCAGCCAGA
ACCTCAAGTGGGAGATGAAACTTGGACTGACCTAGACGGAAAAACCGAAGAGTATGGAGGCAGGGCTCTAAAGCCTA
CTACTAACATGAAACCCTGTTACGGGTCCTATGCGAAGCCTACTAATTTAAAAGGTGGTCAGGCAAAACCGAAAAAC
TCGGAACCGTCGAGTGAAAAAATTGAATATGATATTGACATGGAATTTTTTGATAACTCATCGCAAAGAACAAACTT
CAGTCCTAAAATTGTCATGTATGCAGAAAATGTAGGTTTGGAAACGCCAGACACTCATGTAGTGTACAAACCTGGAA
CAGAAGACACAAGTTCCGAAGCTAATTTGGGACAACAGTCTATGCCCAACAGACCCAACTACATTGGCTTCAGAGAT
AACTTTATTGGACTCATGTACTATAACAGTACTGGTAACATGGGGGTGCTGGCTGGTCAAGCGTCTCAGTTAAATGC
AGTGGTTGACTTGCAGGACAGAAACACAGAACTTTCTTACCAACTCTTGCTTGACTCTCTGGGCGACAGAACCAGAT
ACTTTAGCATGTGGAATCAGGCTGTGGACAGTTATGATCCTGATGTACGTGTTATTGAAAATCATGGTGTGGAAGAT
GAACTTCCCAACTATTGTTTTCCACTGGACGGCATAGGTGTTCCAACAACCAGTTACAAATCAATAGTTCCAAATGG
AGAAGATAATAATAATTGGAAAGAACCTGAAGTAAATGGAACAAGTGAGATCGGACAGGGTAATTTGTTTGCCATGG
AAATTAACCTTCAAGCCAATCTATGGCGAAGTTTCCTTTATTCCAATGTGGCTCTGTATCTCCCAGACTCGTACAAA
TACACCCCGTCCAATGTCACTCTTCCAGAAAACAAAAACACCTACGACTACATGAACGGGCGGGTGGTGCCGCCATC
TCTAGTAGACACCTATGTGAACATTGGTGCCAGGTGGTCTCTGGATGCCATGGACAATGTCAACCCATTCAACCACC
ACCGTAACGCTGGCTTGCGTTACCGATCTATGCTTCTGGGTAACGGACGTTATGTGCCTTTCCACATACAAGTGCCT
CAAAAATTCTTCGCTGTTAAAAACCTGCTGCTTCTCCCAGGCTCCTACACTTATGAGTGGAACTTTAGGAAGGATGT
GAACATGGTTCTACAGAGTTCCCTCGGTAACGACCTGCGGGTAGATGGCGCCAGCATCAGTTTCACGAGCATCAACC
TCTATGCTACTTTTTTCCCCATGGCTCACAACACCGCTTCCACCCTTGAAGCCATGCTGCGGAATGACACCAATGAT
CAGTCATTCAACGACTACCTATCTGCAGCTAACATGCTCTACCCCATTCCTGCCAATGCAACCAATATTCCCATTTC
CATTCCTTCTCGCAACTGGGCGGCTTTCAGAGGCTGGTCATTTACCAGACTGAAAACCAAAGAAACTCCCTCTTTGG
GGTCTGGATTTGACCCCTACTTTGTCTATTCTGGTTCTATTCCCTACCTGGATGGTACCTTCTACCTGAACCACACT
TTTAAGAAGGTTTCCATCATGTTTGACTCTTCAGTGAGCTGGCCTGGAAATGACAGGTTACTATCTCCTAACGAATT
TGAAATAAAGCGCACTGTGGATGGCGAAGGCTACAACGTAGCCCAATGCAACATGACCAAAGACTGGTTCTTGGTAC
AGATGCTCGCCAACTACAACATCGGCTATCAGGGCTTCTACATTCCAGAAGGATACAAAGATCGCATGTATTCATTT
TTCAGAAACTTCCAGCCCATGAGCAGGCAGGTGGTTGATGAGGTCAATTACAAAGACTTCAAGGCCGTCGCCATACC
CTACCAACACAACAACTCTGGCTTTGTGGGTTACATGGCTCCGACCATGCGCCAAGGTCAACCCTATCCCGCTAACT
ATCCCTATCCACTCATTGGAACAACTGCCGTAAATAGTGTTACGCAGAAAAAGTTCTTGTGTGACAGAACCATGTGG
CGCATACCGTTCTCGAGCAACTTCATGTCTATGGGGGCCCTTACAGACTTGGGACAGAATATGCTCTATGCCAACTC
AGCTCATGCTCTGGACATGACCTTTGAGGTGGATCCCATGGATGAGCCCACCCTGCTTTATCTTCTCTTCGAAGTTT
TCGACGTGGTCAGAGTGCATCAGCCACACCGCGGCATCATCGAGGCAGTCTACCTGCGTACACCGTTCTCGGCCGGT
AACGCTACCACGTAAGAAGCTTCTTGCTTCTTGCAAATAGCAGCTGCAACCATGGCCTGCGGATCCCAAAACGGCTC
CAGCGAGCAAGAGCTCAGAGCCATTGTCCAAGACCTGGGTTGCGGACCCTATTTTTTGGGAACCTACGATAAGCGCT
TCCCGGGGTTCATGGCCCCCGATAAGCTCGCCTGTGCCATTGTAAATACGGCCGGACGTGAGACGGGGGGAGAGCAC
TGGTTGGCTTTCGGTTGGAACCCACGTTCTAACACCTGCTACCTTTTTGATCCTTTTGGATTCTCGGATGATCGTCT
CAAACAGATTTACCAGTTTGAATATGAGGGTCTCCTGCGCCGCAGCGCTCTTGCTACCAAGGACCGCTGTATTACGC
TGGAAAAATCTACCCAGACCGTGCAGGGCCCCCGTTCTGCCGCCTGCGGACTTTTCTGCTGCATGTTCCTTCACGCC
TTTGTGCACTGGCCTGACCGTCCCATGGACGGAAACCCCACCATGAAATTGCTAACTGGAGTGCCAAACAACATGCT
TCATTCTCCTAAAGTCCAGCCCACCCTGTGTGACAATCAAAAAGCACTCTACCATTTTCTTAATACCCATTCGCCTT
ATTTTCGCTCTCATCGTACACACATCGAAAGGGCCACTGCGTTCGACCGTATGGATGTTCAATAATGACTCATGTAA
ACAACGTGTTCAATAAACATCACTTTATTTTTTTACATGTATCAAGGCTCTGGATTACTTATTTATTTACAAGTCGA
ATGGGTTCTGACGAGAATCAGAATGACCCGCAGGCAGTGATACGTTGCGGAACTGATACTTGGGTTGCCACTTGAAT
TCGGGAATCACCAACTTGGGAACCGGTATATCGGGCAGGATGTCACTCCACAGCTTTCTGGTCAGCTGCAAAGCTCC
AAGCAGGTCAGGAGCCGAAATCTTGAAATCACAATTAGGACCAGTGCTCTGAGCGCGAGAGTTGCGGTACACCGGAT
TGCAGCACTGAAACACCATCAGCGACGGATGTCTCACGCTTGCCAGCACGGTGGGATCTGCAATCATGCCCACATCC
AGATCTTCAGCATTGGCAATGCTGAACGGGGTCATCTTGCAGGTCTGCCTACCCATGGCGGGCACCCAATTAGGCTT
GTGGTTGCAATCGCAGTGCAGGGGGATCAGTATCATCTTGGCCTGATCCTGTCTGATTCCTGGATACACGGCTCTCA
TGAAAGCATCATATTGCTTGAAAGCCTGCTGGGCTTTACTACCCTCGGGATAAAACATCCCGCAGGACCTGCTCGAA
AACTGGTTAGCCTGCACAGCCGGCATCATTCACACAGCAGCGGGCGTCATTGTTGGCTATTTGCACCACACTTCTGC
CCCAGCGGTTTTGGGTGATTTTGGTTCGCTCGGGATTCTCCTTTAAGGCTCGTTGTCCGTTCTCGCTGGCCACATCC
ATCTCGATAATCTGCTCCTTCTGAATCATAATATTGCCATGCAGGCACTTCAGCTTGCCCTCATAATCATTGCAGCC
ATGAGGCCACAACGCACAGCCTGTACATTCCCAATTATGGTGGGCGATCTGAGAAAAAGAATGTATCATTCCCTGCA
GAAATCTTCCCATCATCGTGCTCAGTGTCTTGTGACTAGTGAAAGTTAACTGGATGCCTCGGTGCTCTTCGTTTACG
TACTGGTGACAGATGCGCTTGTATTGTTCGTGTTGCTCAGGCATTAGTTTAAAACAGGTTCTAAGTTCGTTATCCAG
CCTGTACTTCTCCATCAGCAGACACATCACTTCCATGCCTTTCTCCCAAGCAGACACCAGGGGCAAGCTAATCGGAT
TCTTAACAGTGCAGGCAGCAGCTCCTTTAGCCAGAGGGTCATCTTTAGCGATCTTCTCAATGCTTCTTTTGCCATCC
TTCTCAACGATGCGCACGGGCGGGTAGCTGAAACCCACTGCTACAAGTTGCGCCTCTTCTCTTTCTTCTTCGCTGTC
TTGACTGATGTCTTGCATGGGGATATGTTTGGTCTTCCTTGGCTTCTTTTTGGGGGGTATCGGAGGAGGAGGACTGT
CGCTCCGTTCCGGAGACAGGGAGGATTGTGACGTTTCGCTCACCATTACCAACTGACTGTCGGTAGAAGAACCTGAC
CCCACACGGCGACAGGTGTTTTTCTTCGGGGGCAGAGGTGGAGGCGATTGCGAAGGGCTGCGGTCCGACCTGGAAGG
CGGATGACTGGCAGAACCCCTTCCGCGTTCGGGGGTGTGCTCCCTGTGGCGGTCGCTTAACTGATTTCCTTCGCGGC
TGGCCATTGTGTTCTCCTAGGCAGAGAAACAACAGACATGGAAACTCAGCCATTGCTGTCAACATCGCCACGAGTGC
CATCACATCTCGTCCTCAGCGACGAGGAAAAGGAGCAGAGCTTAAGCATTCCACCGCCCAGTCCTGCCACCACCTCT
ACCCTAGAAGATAAGGAGGTCGACGCATCTCATGACATGCAGAATAAAAAAGCGAAAGAGTCTGAGACAGACATCGA
GCAAGACCCGGGCTATGTGACACCGGTGGAACACGAGGAAGAGTTGAAACGCTTTCTAGAGAGAGAGGATGAAAACT
GCCCAAAACAGCGAGCAGATAACTATCACCAAGATGCTGGAAATAGGGATCAGAACACCGACTACCTCATAGGGCTT
GACGGGGAAGACGCGCTCCTTAAACATCTAGCAAGACAGTCGCTCATAGTCAAGGATGCATTATTGGACAGAACTGA
AGTGCCCATCAGTGTGGAAGAGCTCAGCTGCGCCTACGAGCTTAACCTTTTTTCACCTCGTACTCCCCCCAAACGTC
AGCCAAACGGCACCTGCGAGCCAAATCCTCGCTTAAACTTTTATCCAGCTTTTGCTGTGCCAGAAGTACTGGCTACC
TATCACATCTTTTTTAAAAATCAAAAAATTCCAGTCTCCTGCCGCGCTAATCGCACCCGCGCCGATGCCCTACTCAA
TCTGGGACCTGGTTCACGCTTACCTGATATAGCTTCCTTGGAAGAGGTTCCAAAGATCTTCGAGGGTCTGGGCAATA
ATGAGACTCGGGCCGCAAATGCTCTGCAAAAGGGAGAAAATGGCATGGATGAGCATCACAGCGTTCTGGTGGAATTG
GAAGGCGATAATGCCAGACTCGCAGTACTCAAGCGAAGCGTCGAGGTCACACACTTCGCATATCCCGCTGTCAACCT
GCCCCCTAAAGTCATGACGGCGGTCATGGACCAGTTACTCATTAAGCGCGCAAGTCCCCTTTCAGAAGACATGCATG
ACCCAGATGCCTGTGATGAGGGTAAACCAGTGGTCAGTGATGAGCAGCTAACCCGATGGCTGGGCACCGACTCTCCC
CGGGATTTGGAAGAGCGTCGCAAGCTTATGATGGCCGTGGTGCTGGTTACCGTAGAACTAGAGTGTCTCCGACGTTT
CTTTACCGATTCAGAAACCTTGCGCAAACTCGAAGAGAATCTGCACTACACTTTTAGACACGGCTTTGTGCGGCAGG
CATGCAAGATATCTAACGTGGAACTCACCAACCTGGTTTCCTACATGGGTATTCTGCATGAGAATCGCCTAGGACAA
AGCGTGCTGCACAGCACCCTTAAGGGGGAAGCCCGCCGTGATTACATCCGCGATTGTGTCTATCTCTACCTGTGCCA
CACGTGGCAAACCGGCATGGGTGTATGGCAGCAATGTTTAGAAGAACAGAACTTGAAAGAGCTTGACAAGCTCTTAC
AGAAATCTCTTAAGGTTCTGTGGACAGGGTTCGACGAGCGCACCGTCGCTTCCGACCTGGCAGACCTCATCTTCCCA
GAGCGTCTCAGGGTTACTTTGCGAAACGGATTGCCTGACTTTATGAGCCAGAGCATGCTTAACAATTTTCGCTCTTT
CATCCTGGAACGCTCCGGTATCCTGCCCGCCACCTGCTGCGCACTGCCCTCCGACTTTGTGCCTCTCACCTACCGCG
AGTGCCCCCCGCCGCTATGGAGTCACTGCTACCTGTTCCGTCTGGCCAACTATCTCTCCTACCACTCGGATGTGATC
GAGGATGTGAGCGGAGACGGCTTGCTGGAGTGCCACTGCCGCTGCAATCTGTGCACGCCCCACCGGTCCCTAGCTTG
CAACCCCCAGTTGATGAGCGAAACCCAGATAATAGGCACCTTTGAATTGCAAGGCCCCAGCAGCCAAGGCGATGGGT
CTTCTCCTGGGCAAAGTTTAAAACTGACCCCGGGACTGTGGACCTCCGCCTACTTGCGCAAGTTTGCTCCGGAAGAT
TACCACCCCTATGAAATCAAGTTCTATGAGGACCAATCACAGCCTCCAAAGGCCGAACTTTCGGCTTGCGTCATCAC
CCAGGGGGCAATTCTGGCCCAATTGCAAGCCATCCAAAAATCCCGCCAAGAATTTCTACTGAAAAAGGGTAAGGGGG
TCTACCTTGACCCCCAGACCGGCGAGGAACTCAACACAAGGTTCCCTCAGGATGTCCCAACGACGAGAAAACAAGAA
GTTGAAGGTGCAGCCGCCGCCCCCAGAAGATATGGAGGAAGATTGGGACAGTCAGGCAGAGGAGGCGGAGGAGGACA
GTCTGGAGGACAGTCTGGAGGAAGACAGTTTGGAGGAGGAAAACGAGGAGGCAGAGGAGGTGGAAGAAGTAACCGCC
GACAAACAGTTATCCTCGGCTGCGGAGACAAGCAACAGCGCTACCATCTCCGCTCCGAGTCGAGGAACCCGGCGGCG
TCCCAGCAGTAGATGGGACGAGACCGGACGCTTCCCGAACCCAACCAGCGCTTCCAAGACCGGTAAGAAGGATCGGC
AGGGATACAAGTCCTGGCGGGGGCATAAGAATGCCATCATCTCCTGCTTGCATGAGTGCGGGGGCAACATATCCTTC
ACGCGGCGCTACTTGCTATTCCACCATGGGGTGAACTTTCCGCGCAATGTTTTGCATTACTACCGTCACCTCCACAG
CCCCTACTATAGCCAGCAAATCCCGACAGTCTCGACAGATAAAGACAGCGGCGGCGACCTCCAACAGAAAACCAGCA
GCGGCAGTTAGAAAATACACAACAAGTGCAGCAACAGGAGGATTAAAGATTACAGCCAACGAGCCAGCGCAAACCCG
AGAGTTAAGAAATCGGATCTTTCCAACCCTGTATGCCATCTTCCAGCAGAGTCGGGGTCAAGAGCAGGAACTGAAAA
TAAAAAACCGATCTCTGCGTTCGCTCACCAGAAGTTGTTTGTATCACAAGAGCGAAGATCAACTTCAGCGCACTCTC
GAGGACGCCGAGGCTCTCTTCAACAAGTACTGCGCGCTGACTCTTAAAGAGTAGGCAGCGACCGCGCTTATTCAAAA
AAGGCGGGAATTACATCATCCTCGACATGAGTAAAGAAATTCCCACGCCTTACATGTGGAGTTATCAACCCCAAATG
GGATTGGCAGCAGGCGCCTCCCAGGACTACTCCACCCGCATGAATTGGCTCAGCGCCGGGCCTTCTATGATTTCTCG
AGTTAATGATATACGCGCCTACCGAAACCAAATACTTTTGGAACAGTCAGCTCTTACCACCACGCCCCGCCAACACC
TTAATCCCAGAAATTGGCCCGCCGCCCTAGTGTACCAGGAAAGTCCCGCTCCCACCACTGTATTACTTCCTCGAGAC
GCCCAGGCCGAAGTCCAAATGACTAATGCAGGTGCGCAGTTAGCTGGCGGCTCCACCCTATGTCGTCACAGGCCTCG
GCATAATATAAAACGCCTGATGATCAGAGGCCGAGGTATCCAGCTCAACGACGAGTCGGTGAGCTCTCCGCTTGGTC
TACGACCAGACGGAATCTTTCAGATTGCCGGCTGCGGGAGATCTTCCTTCACCCCTCGTCAGGCTGTTCTGACTTTG
GAAAGTTCGTCTTCGCAACCCCGCTCGGGCGGAATCGGGACCGTTCAATTTGTAGAGGAGTTTACTCCCTCTGTCTA
CTTCAACCCCTTCTCCGGATCTCCTGGGCACTACCCGGACGAGTTCATACCGAACTTCGACGCGATTAGCGAGTCAG
TGGACGGCTACGATTGATGTCTGGTGACGCGGCTGAGCTATCTCGGCTGCGACATCTAGACCACTGCCGCCGCTTTC
GCTGCTTTGCCCGGGAACTTATTGAGTTCATCTACTTCGAACTCCCCAAGGATCACCCTCAAGGTCCGGCCCACGGA
GTGCGGATTACTATCGAAGGCAAAATAGACTCTCGCCTGCAACGAATTTTCTCCCAGCGGCCCGTGCTGATCGAGCG
AGACCAGGGAAACACCACGGTTTCCATCTACTGCATTTGTAATCACCCCGGATTGCATGAAAGCCTTTGCTGTCTTA
TGTGTACTGAGTTTAATAAAAACTGAATTAAGACTCTCCTACGGACTGCCGCTTCTTCAACCCGGATTTTACAACCA
GAAGAACAAAACTTTTCCTGTCGTCCAGGACTCTGTTAACTTCACCTTTCCTACTCACAAACTAGAAGCTCAACGAC
TACACCGCTTTTCCAGAAGCATTTTCCCTACTAATACTACTTTCAAAACCGGAGGTGAGCTCCACGGTCTCCCTACA
GAAAACCCTTGGGTGGAAGCGGGCCTTGTAGTACTAGGAATTCTTGCGGGTGGGCTTGTGATTATTCTTTGCTACCT
ATACACACCTTGCTTCACTTTCCTAGTGGTGTTGTGGTATTGGTTTAAAAAATGGGGCCCATACTAGTCTTGCTTGT
TTTACTTTCGCTTTTGGAACCGGGTTCTGCCAATTACGATCCATGTCTAGACTTTGACCCAGAAAACTGCACACTTA
CTTTTGCACCCGACACAAGCCGCATCTGTGGAGTTCTTATTAAGTGCGGATGGGAATGCAGGTCCGTTGAAATTACA
CACAATAACAAAACCTGGAACAATACCTTATCCACCACATGGGAGCCAGGAGTTCCCGAGTGGTACACTGTCTCTGT
CCGAGGTCCTGACGGTTCCATCCGCATTAGTAACAACACTTTCATTTTTTCTGAAATGTGCGATCTGGCCATGTTCA
TGAGCAAACAGTATTCTCTATGGCCTCCTAGCAAGGACAACATCGTAACGTTCTCCATTGCTTATTGCTTGTGCGCT
TGCCTTCTTACTGCTTTACTGTGCGTATGCATACACCTGCTTGTAACCACTCGCATCAAAAACGCCAATAACAAAGA
AAAAATGCCTTAACCTCTTTCTGTTTACAGACATGGCTTCTCTTACATCTCTCATATTTGTCAGCATTGTCACTGCC
GCTCACGGACAAACAGTCGTCTCTATCCCACTAGGACATAATTACACTCTCATAGGACCCCCAATCACTTCAGAGGT
CATCTGGACCAAACTGGGAAGCGTTGATTACTTTGATATAATCTGTAACAAAACAAAACCAATAATAGTAACTTGCA
ACATACAAAATCTTACATTGATTAATGTTAGCAAAGTTTACAGCGGTTACTATTATGGTTATGACAGATACAGTAGT
CAATATAGAAATTACTTGGTTCGTGTTACCCAGTTGAAAACCACGAAAATGCCAAATATGGCAAAGATTCGATCCGA
TGACAATTCTCTAGAAACTTTTACATCTCCCACCACACCCGACGAAAAAAACATCCCAGATTCAATGATTGCAATTG
TTGCAGCGGTGGCAGTGGTGATGGCACTAATAATAATATGCATGCTTTTATATGCTTGTCGCTACAAAAAGTTTCAT
CCTAAAAAACAAGATCTCCTACTAAGGCTTAACATTTAATTTCTTTTTATACAGCCATGGTTTCCACTACCACATTC
CTTATGCTTACTAGTCTCGCAACTCTGACTTCTGCTCGCTCACACCTCACTGTAACTATAGGCTCAAACTGCACACT
AAAAGGACCTCAAGGTGGTCATGTCTTTTGGTGGAGAATATATGACAATGGATGGTTTACAAAACCATGTGACCAAC
CTGGTAGATTTTTCTGCAACGGCAGAGACCTAACCATTATCAACGTGACAGCAAATGACAAAGGCTTCTATTATGGA
ACCGACTATAAAAGTAGTTTAGATTATAACATTATTGTACTGCCATCTACCACTCCACCACCCCGCACAACTACTTT
CTCTAGCAGCAGTGTCGCTAACAATACAATTTCCAATCCAACCTTTGCCGCGCTTTTAAAACGCACTGTGAATAATT
CTACAACTTCACATACAACAATTTCCACTTCAACAATCAGCATCATCGCTGCAGTGACAATTGGAATATCTATTCTT
GTTTTTACCATAACCTACTACGCCTGCTGCTATAGAAAAGACAAACATAAAGGTGATCCATTACTTAGATTTGATAT
TTAATTTGTTCTTTTTTTTTATTTACAGTATGGTGAACACCAATCATGGTACCTAGAAATTTCTTCTTCACCATACT
CATCTGTGCTTTTAATGTTTGCGCTACTTTCACAGCAGTAGCCACAGCAACCCCAGACTGTATAGGAGCATTTGCTT
CCTATGCACTTTTTGCTTTTGTTACTTGCATCTGCGTATGTAGCATAGTCTGCCTGGTTATTAATTTTTTCCAACTT
CTAGACTGGATCCTTGTGCGAATTGCCTACCTGCGCCACCATCCCGAATACCGCAACCAAAATATCGCGGCACTTCT
TAGACTCATCTAAAACCATGCAGGCTATACTACCAATATTTTTGCTTCTATTGCTTCCCTACGCTGTCTCAACCCCA
GCTGCCTATAGTACTCCACCAGAACACCTTAGAAAATGCAAATTCCAACAACCGTGGTCATTTCTTGCTTGCTATCG
AGAAAAATCAGAAATCCCCCCAAATTTAATAATGATTGCTGGAATAATTAATATAATCTGTTGCACCATAATTTCAT
TTTTGATATACCCCCTATTTGATTTTGGCTGGAATGCTCCCAATGCACATGATCATCCACAAGACCCAGAGGAACAC
ATTCCCCCACAAAACATGCAACATCCAATAGCGCTAATAGATTACGAAAGTGAACCACAACCCCCACTACTCCCTGC
TATTAGTTACTTCAACCTAACCGGCGGAGATGACTGAAACACTCACCACCTCCAATTCCGCCGAGGATCTGCTCGAT
ATGGACGGCCGCGTCTCAGAACAACGACTTGCCCAACTACGCATCCGCCAGCAGCAGGAACGCGTGGCCAAAGAGCT
CAGAGATGTCATCCAAATTCACCAATGCAAAAAAGGCATATTCTGTTTGGTAAAACAAGCCAAGATATCCTACGAGA
TCACCGCTACTGACCATCGCCTCTCTTACGAACTTGGCCCCCAACGACAAAAATTTACCTGCATGGTGGGAATCAAC
CCCATAGTTATCACCCAACAAAGTGGAGATACTAAGGGTTGCATTCACTGTTCCTGCGATTCCATCGAGTGCACCTA
CACCCTGCTGAAGACCCTATGCGGCCTAAGAGACCTGCTACCAATGAATTAAAAAAAAATGATTAATAAAAAATCAC
TTACTTGAAATCAGCAATAAGGTCTCTGTTGAAATTTTCTCCCAGCAGCACCTCACTTCCCTCTTCCCAACTCTGGT
ATTCTAAACCCCGTTCAGCGGCATACTTTCTCCATACTTTAAAGGGGATGTCAAATTTTAGCTCCTCTCCTGTACCC
ACAATCTTCATGTCTTTCTTCCCAGATGACCAAGAGAGTCCGGCTCAGTGACTCCTTCAACCCTGTCTACCCCTATG
AAGATGAAAGCACCTCCCAACACCCCTTTTATAACCCAGGGTTTATTTCCCCAAATGGCTTCACACAAAGCCCAGAC
GGAGTTCTTACTTTAAAATGTTTAACCCCACTAACAACCACAGGCGGATCTCTACAGCTAAAAGTGGGAGGGGGACT
TACAGTGGATGACACTGATGGTACCTTACAAGAAAACATACGTGCTACAGCACCCATTACTAAAAATAATCACTCTG
TAGAACTATCCATTGGAAATGGATTAGAAACTCAAAACAATAAACTATGTGCCAAATTGGGAAATGGGTTAAAATTT
AACAACGGTGACATTTGTATAAAGGATAGTATTAACACCTTATGGACTGGAATAAACCCTCCACCTAACTGTCAAAT
TGTGGAAAACACTAATACAAATGATGGCAAACTTACTTTAGTATTAGTAAAAAATGGAGGGCTTGTTAATGGCTACG
TGTCTCTAGTTGGTGTATCAGACACTGTGAACCAAATGTTCACACAAAAGACAGCAAACATCCAATTAAGATTATAT
TTTGACTCTTCTGGAAATCTATTAACTGAGGAATCAGACTTAAAAATTCCACTTAAAAATAAATCTTCTACAGCGAC
CAGTGAAACTGTAGCCAGCAGCAAAGCCTTTATGCCAAGTACTACAGCTTATCCCTTCAACACCACTACTAGGGATA
GTGAAAACTACATTCATGGAATATGTTACTACATGACTAGTTATGATAGAAGTCTATTTCCCTTGAACATTTCTATA
ATGCTAAACAGCCGTATGATTTCTTCCAATGTTGCCTATGCCATACAATTTGAATGGAATCTAAATGCAAGTGAATC
TCCAGAAAGCAACATAGCTACGCTGACCACATCCCCCTTTTTCTTTTCTTACATTACAGAAGACGACAACTAAAATA
AAGTTTAAGTGTTTTTATTTAAAATCACAAAATTCGAGTAGTTATTTTGCCTCCACCTTCCCATTTGACAGAATACA
CCAATCTCTCCCCACGCACAGCTGTTAAACATTTGGATACCATTAGAGATAGACATTGTTTTAGATTCCACATTCCA
AACAGTTTCAGAGCGAGCCAATCTGGGGTCAGTGATAGATAAAAATCCATCGCGATAGTCTTTTAAAGCGCTTTCAC
AGTCCAACTGCTGCGGATGCGACTCCGGAGTTTGGATCACGGTCATCTGGAAGAAGAACGATGGGAATCATAATCCG
AAAACGGTATCGGACGATTGTGTCTCATCAAACCCACAAGCAGCCGCTGTCTGCGTCGCTCCGTGCGACTGCTGTTT
ATGGGATCAGGGTCCACAGTTTCCTGAAGCATGATTTTAATAGCCCTTAACATCAACTTTCTGGTGCGATGCGCGCA
GCAACGCATTCTGATTTCACTCAAATCTTTGCAGTAGGTACAACACATTATTACAATATTGTTTAATAAACCATAAT
TAAAAGCGCTCCAGCCAAAACTCATATCTGATATAATCGCCCCTGCATGACCATCATACCAAAGTTTAATATAAATT
AAATGACGTTCCCTCAAAAACACACTACCCACATACATGATCTCTTTTGGCATGTGCATATTAACAATCTGTCTGTA
CCATGGACAACGTTGGTTAATCATGCAACCCAATATAACCTTCCGGAACCACACTGCCAACACCGCTCCCCCAGCCA
TGCATTGAAGTGAACCCTGCTGATTACAATGACAATGAAGAACCCAATTCTCTCGACCGTGAATCACTTGAGAATGA
AAAATATCTATAGTGGCACAACATAGACATAAATGCATGCATCTTCTCATAATTTTTAACTCCTCAGGATTTAGAAA
CATATCCCAGGGAATAGGAAGCTCTTGCAGAACAGTAAAGCTGGCAGAACAAGGAAGACCACGAACACAACTTACAC
TATGCATAGTCATAGTATCACAATCTGGCAACAGCGGGTGGTCTTCAGTCATAGAAGCTCGGGTTTCATTTTCCTCA
CAACGTGGTAACTGGGCTCTGGTGTAAGGGTGATGTCTGGCGCATGATGTCGAGCGTGCGCGCAACCTTGTCATAAT
GGAGTTGCTTCCTGACATTCTCGTATTTTGTATAGCAAAACGCGGCCCTGGCAGAACACACTCTTCTTCGCCTTCTA
TCCTGCCGCTTAGCGTGTTCCGTGTGATAGTTCAAGTACAGCCACACTCTTAAGTTGGTCAAAAGAATGCTGGCTTC
AGTTGTAATCAAAACTCCATCGCATCTAATTGTTCTGAGGAAATCATCCACGGTAGCATATGCAAATCCCAACCAAG
CAATGCAACTGGATTGCGTTTCAAGCAGGAGAGGAGAGGGAAGAGACGGAAGAACCATGTTAATTTTTATTCCAAAC
GATCTCGCAGTACTTCAAATTGTAGATCGCGCAGATGGCATCTCTCGCCCCCACTGTGTTGGTGAAAAAGCACAGCT
AAATCAAAAGAAATGCGATTTTCAAGGTGCTCAACGGTGGCTTCCAACAAAGCCTCCACGCGCACATCCAAGAACAA
AAGAATACCAAAAGAAGGAGCATTTTCTAACTCCTCAATCATCATATTACATTCCTGCACCATTCCCAGATAATTTT
CAGCTTTCCAGCCTTGAATTATTCGTGTCAGTTCTTGTGGTAAATCCAATCCACACATTACAAACAGGTCCCGGAGG
GCGCCCTCCACCACCATTCTTAAACACACCCTCATAATGACAAAATATCTTGCTCCTGTGTCACCTGTAGCGAATTG
AGAATGGCAACATCAATTGACATGCCCTTGGCTCTAAGTTCTTCTTTAAGTTCTAGTTGTAAAAACTCTCTCATATT
ATCACCAAACTGCTTAGCCAGAAGCCCCCCGGGAACAAGAGCAGGGGACGCTACAGTGCAGTACAAGCGCAGACCTC
CCCAATTGGCTCCAGCAAAAACAAGATTGGAATAAGCATATTGGGAACCACCAGTAATATCATCGAAGTTGCTGGAA
ATATAATCAGGCAGAGTTTCTTGTAGAAATTGAATAAAAGAAAAATTTGCCAAAAAAACATTCAAAACCTCTGGGAT
GCAAATGCAATAGGTTACCGCGCTGCGCTCCAACATTGTTAGTTTTGAATTAGTCTGCAAAAATAAAAAAAAAACAA
GCGTCATATCATAGTAGCCTGACGAACAGGTGGATAAATCAGTCTTTCCATCACAAGACAAGCCACAGGGTCTCCAG
CTCGACCCTCGTAAAACCTGTCATCGTGATTAAACAACAGCACCGAAAGTTCCTCGCGGTGACCAGCATGAATAAGT
CTTGATGAAGCATACAATCCAGACATGTTAGCATCAGTTAAGGAGAAAAAACAGCCAACATAGCCTTTGGGTATAAT
TATGCTTAATCGTAAGTATAGCAAAGCCACCCCTCGCGGATACAAAGTAAAAGGCACAGGAGAATAAAAAATATAAT
TATTTCTCTGCTGCTGTTTAGGCAACGTCGCCCCCGGTCCCTCTAAATACACATACAAAGCCTCATCAGCCATGGCT
TACCAGAGAAAGTACAGCGGGCACACAAACCACAAGCTCTAAAGTCACTCTCCAACCTCTCCACAATATATATACAC
AAGCCCTAAACTGACGTAATGGGACTAAAGTGTAAAAAATCCCGCCAAACCCAACACACACCCCGAAACTGCGTCAC
CAGGGAAAAGTACAGTTTCACTTCCGCAATCCCAACAAGCGTCACTTCCTCTTTCTCACGGTACGTCACATCCCATT
AACTTACAACGTCATTTTCCCACGGCCGCGCCGCCCCTTTTAACCGTTAACCCCACAGCCAATCACCACACGGCCCA
CACTTTTTAAAATCACCTCATTTACATATTGGCACCATTCCATCTATAAGGTATATTATTGATGATG

GenBank Accession No. AP 000580 (SEQ ID NO: 244)

MRRVVLGGAVVYPEGPPPSYESVMQQQQATAVMQSPLEAPFVPPRYLAP
TEGRNSIRYSELAPQYDTTRLYLVDNKSADIASLNYQNDHSNFLTTVVQ
NNDFTPTEASTQTINFDERSRWGGQLKTIMHTNMPNVNEYMFSNKFKAR
VMVSRKPPDGAAVGDTYDHKQDILEYEWFEFTLPEGNFSVTMTIDLMNN
AIIDNYLKVGRQNGVLESDIGVKFDTRNFKLGWDPETKLIMPGVYTYEA
FHPDIVLLPGCGVDFTESRLSNLLGIRKKQPFQEGFKILYEDLEGGNIP
ALLDVDAYENSKKEQKAKIEAATAAAEAKANIVASDSTRVANAGEVRGD
NFAPTPVPTAESLLADVSEGTDVKLTIQPVEKDSKNRSYNVLEDKINTA
YRSWYLSYNYGDPEKGVRSWTLLTTSDVTCGAEQVYWSLPDMMKDPVTF
RSTRQVSNYPVVGAELMPVFSKSFYNEQAVYSQQLRQSTSLTHVFNRFP
ENQILIRPPAPTITTVSENVPALTDHGTLPLRSSIRGVQRVTVTDARRR
TCPYVYKALGIVAPRVLSSRTF

GenBank Accession No. AP_000585 (SEQ ID NO: 245)

MATPSMLPQWAYMHIAGQDASEYLSPGLVQFARATDTYFNLGNKFRNPT
VAPTHDVTTDRSQRLMLRFVPVDREDNTYSYKVRYTLAVGDNRVLDMAS
TFFDIRGVLDRGPSFKPYSGTAYNSLAPKGAPNASQWIAKGVPTAAAAG
NGEEEHETEEKTATYTFANAPVKAEAQITKEGLPIGLEISAENESKPIY
ADKLYQPEPQVGDETWTDLDGKTEEYGGRALKPTTNMKPCYGSYAKPTN
LKGGQAKPKNSEPSSEKIEYDIDMEFFDNSSQRTNFSPKIVMYAENVGL
ETPDTHVVYKPGTEDTSSEANLGQQSMPNRPNYIGFRDNFIGLMYYNST
GNMGVLAGQASQLNAVVDLQDRNTELSYQLLLDSLGDRTRYFSMWNQAV
DSYDPDVRVIENHGVEDELPNYCFPLDGIGVPTTSYKSIVPNGEDNNNW
KEPEVNGTSEIGQGNLFAMEINLQANLWRSFLYSNVALYLPDSYKYTPS
NVTLPENKNTYDYMNGRVVPPSLVDTYVNIGARWSLDAMDNVNPFNHHR
NAGLRYRSMLLGNGRYVPFHIQVPQKFFAVKNLLLLPGSYTYEWNFRKD
VNMVLQSSLGNDLRVDGASISFTSINLYATFFPMAHNTASTLEAMLRND
TNDQSFNDYLSAANMLYPIPANATNIPISIPSRNWAAFRGWSFTRLKTK
ETPSLGSGFDPYFVYSGSIPYLDGTFYLNHTFKKVSIMFDSSVSWPGND
RLLSPNEFEIKRTVDGEGYNVAQCNMTKDWFLVQMLANYNIGYQGFYIP
EGYKDRMYSFFRNFQPMSRQVVDEVNYKDFKAVAIPYQHNNSGFVGYMA
PTMRQGQPYPANYPYPLIGTTAVNSVTQKKFLCDRTMWRIPFSSNFMSM
GALTDLGQNMLYANSAHALDMTFEVDPMDEPTLLYLLFEVFDVVRVHQP
HRGIIEAVYLRTPFSAGNATT

GenBank Accession No. Z84721 (SEQ ID NO: 246)

GATCACGCCATTGCACTCCACCCTGGGCGACAGAGCGACGAGACCCCGTATCAAAAAAAAAAAAAAGAAAGAAAGAA
AGAAAAAAGAAAAAAAAAAGGCCGGGCGCGGTGGCTCACGCCTGTAATCCCAGCACTTTGGGAGGCCGAGGCGGGTG
AATCACGAGGTCAGGAGTTCGAGACCATCCTGGCCAACATGGTGAAACCCCGTCTCTACAAAAAAAAAAAAAAAAAT
TAGCCGGGCGTGGTGGCGGGCGCCTGTAATCCCAGCTACTCGGGAGGCTGAGACAGGAAAATCGCTTGAACCCGGGA
GGCGGAGCTTGCGGTGAGCCGAGATTGCGCCACTGCACTACAGCCTAGGCGACAGAGCGAGACTCCGTCTCAAAAAA
AAAAAAAAAAAAAAAAAACACTTGGAAGCCGACAGGAGATCTTTGAGACCTTGGGCGAGGCAGTGACACTAAAGGCA
GGAGCGACTACAGAAGAATAAATTAAACTTCATCAGATTAAAAACTTTACTGCGGCCGGGCGCGGTGGCTCACGCCT
GAAATCCCAGCACTTTGGGAGGCCGAGGTGGGCAGATCATGAGATCAGGAGATCTAGACCATCCTGGCCAACATGGT
AAAACCCCGTCTCTCTACTAAAAATACAAAAATTAGCTGGGTTTGGCGGCGCCTGCTTCTAATCCCAGCTACTCGGG
AGGCTGAGGCAGGAGAATCGCTTGAAGCCGGGAGGCGGAGGTTGCAGTGAGCCGAGATCGTGCCACTGAACTCTGGC
CTGGCGACAGAGCGAGACTCCATCTCAAAACAAAACAAAAACTTCGGTGCTTTAAAGGACACCATCAAGAAAATTAA
AAGTCCACCCACAGAACGGGAGAAAATATTTGTAAGTTACATATCTGATAAGGGAATTGTATCTAGAATGGAGGAAA
CTTACAACTCAACAATAAAAAGACAATTGAAAAATGCACAAAGGATATGAATATTTTTCCAGTGCATTATGCAAATG
GCCAATAAGCACCAGAAGATGCTCAGCTCAACTGGTAGAGGCTTACGCCTGTGACCCCAGCGCTGAGAGGCCAGGAA
CTCCAGACCAGCCTGGGCAAAACAGAAATTAAAAATGCTCAACATTATTAGGCATTAGGGAGATGCAAATCAAAACT
ACAAATAGATGCCACATCACACCTCCTACGATGGCTGTAATCAAAAAGACAAGCGTCAGCAGGGGTGTGGAGAAACG
GGAATCTCTCTCCTGCTGGTGGGAATGTAAGAGGCTACACTCGCTATGGAAAACAGGCTGGCAGTTCCTGAAAGGTT
AGAGTTAACACAACACTCGGCAAATCCCCCTTTTAGATATATAGCCAAGAGAAATGAAAGCATATGTCCACACAAAA
ACATGTGTGTTCTTAGTAATATTATTCATAATAGCCCAAAGTGGAAGCAATCCTAGGGTATATCAATTGATGAATGG
GTGAATATGGTATAGTTTGTTTAAGGGAATACTATTCAGCCATAAAAAGGAATGAAGTACGGCACATGAATCCATCT
TGAAGACACACTAATATATGATTCCATTTATATAAGATGCCCAGAATAGGCAAATCCATAGAGACAGAATGATTAGT
GGCTGCCTAGGGCTTCCAGGGGGTCAGGGGAAATATGGAGCGATTCATGGGTTTTTTGAAGGGGAGTGATGAAAATG
TTCTAACGTTGACTGTGGTAATGGTTGGACAGCTCTGAGAACGCGAATACACTAAAAGACATGGAAGTGCCGGGCGC
AGTGGCTCATGCCTGTAATCCCAGCGCTTTGGGAGGCCAAGGCAGGCGGATCGCGAGGTCAGGAGATCGAGACCATC
CTGGCTAAGACAGTGAAACCCCGTGTCTACTAAAAATACAAAAAATTAGCTGGACATGGTGCGGGCGCCTGTAGTCC
CAGATACTCAGGAGGCTGAGGCAGGAGAATGGTGTGAACCCGGGAGGCGGAGCTTGCAGTGAGCCAAGATCGCACCA
TTGCACTCCAGCCTGGGCGACAGAGCGAGACTCCATCTCAAAAACAAAAAAAAGATATGGAAGTGTACACTTGAAGT
GGATAAGCTTTATGGTATGCAAATTGGTATGGTATGGTAAATTATATCTCAATGAAGTTGTTTTTTAAAAAATCACC
CCACCTACCCTATCCCAGGCTTCCCCAGGAGGTAACTAAAGGTAATGAGCTTCTTTGGCTGCTTCCAGAACTTTCCC
AAGCACATCAAATGCATCAGAACCTAACCACTTGACTGAGGGATGAGCATTTTCACTGTTGCAAGTAACCCTCTTGC
ACCAACACTGACACTAATGTGTATTTTGCAGAACAAATTTGTGGATTGGCCTCACCAGGGTGAAGGGTACGTGCATT
TGAAATGGCTCAACAGTACCAACAGGTGCGTTTTCTTGCACAGGGCTGCATAACATTTTTTTTTTTTTTTTGAGACA
GAGTCTCGCTCTATCACCCAGGCTGGAGGGCAGTGGCACAATCTCAGTTCACTGCAAGCTCCACCTACCAGGTTCAC
ATCATTCTCCTGCCTCAGCCTCCCAAGTAGCTGGGACTACAGGTGCCCGCCACCACACCAGGCTAATTTTTTTTTTT
TTTTTGAGATGGAGTCTTGCTCTGTCGCCCAGGCTGGAGTGCAGTGGCACGATCTCAGCTCACTGCAAGCTCCACCT
CCCAGGTTCACACCATTCTCCTGCCTCAGCCTCCCCAGTAGCTGAGACTACAGGCGCCCGCCACCACGTCCGGCTAA
TTTTTTTGTATTTTTAGTAGAGACGGGGTTTCACCGCGTTAGCCAGGATGGTCTCGATCTCCTGACCTCTTGATCCA
CCCGCCTCGGCCTCTCAAAGTGCTGGGATTACAGGCGTGAGCCACCGTGCCCGGCCTGCATAACATTTTTTTTTTTC
CTGAAATTCCCAGAAAGGAAAATGGTGTCTTGTTCTATGTTGCATTTCTTTGATTGAGAGGGAGAGCTGCATCACTT
AATTATTTGCAGAGAATTGCTTTTCTTGTTTTCTTTACAGGTGGTCTGTTCTTGGATGGTCTGGCTGTGTTCTTTCT
GAGGAATACATAACCTCTGCTACACATTTTGCAAGGCTTTATCCCCGTTGTCCATGTTTTGATTTTATGTATAATCA
AAAGGTTTGTGAGTTCTCCCGCACTTCCCAGGAGTGCCTCTGGGATGGAAATGAGACTGCAGGAGCAGGGCTTGAGG
CTGGAGGGGTGAGATGGGACAGATGGGGGTGGGGGAACCCAGGGCAGTGGCCGGTGGTGGTAATGGAGGCCTCCTCA
CAGGGACCCTCACAGCGACCATGCGAATGGAGCAGGACTGTGACTCAGGTCTCGCTCTTCTGACCTAATCGTGCTGC
TGCCCCAATGGGCAGAACCTTGGGGCTCCAGACTGGACATCTCTGGGCTCAAAGGATCCCACTGTTCCCCCGGTTAC
CCTCTCAGGGTTGGCCTCCTGCCAGTAACCCTGGCACTCATTGTTCATTCTTCTGACTATCGTCAGTCATAATGAGA
GCTCGAACTGGTGAAAGTGCAGGGAGCTCACCATGACCCCAGCCCACAGAGGTCCTGGGTGCGTCCCTGCCCTCGAA
GCAGCACTCTGGATCCCAGCGCCACCCTCATGTCCATGTTTGCACCTCATTGGCTGTGACAGAAATGAGACATCATT
GTCACACGCTGGCCTGAGGGTCAGTGGGCCTTGCTTTGGACCTCAGTTTCCCCACCAGTAACAGGGTTCAGAGCAGA
TGGTCCCTGAGTGAGTCCCAGCTCTAAGTTCTCCCAGGGTCTCCTGGACAATGAAGCACCAGGGCCAACCTCCATTT
GCTACAGGGGACATCCTCAGGCTCTTCTCTGCTAAGACCCCACACCTCCAAGTCTCCTCATTTTACCTTTAAATAGC
TGTTTCATGACCTGCTTTTTTGACGGTAAGTAGATTTTTGGAAACTGAAACCCCTGACCCTTCCTCCCAGCCTGGGC
CTGCCCTTGGCAGGATAGGAGGCCTTATCGGTCCTGCCACTTGGTCTGGGCCTCAAAGGGCCACCGCCATCTGCAGG
AGGGCCGGGTGGGGTTCACAGACGCTATCTGGGACTTGCCTGGACACCTCCACCTTCTCAGCTGAGTGTTGCTGCCC
CACCAGGGAGAACCACTCACACACAGTAGTAATAGAAATAATTTAAAATTCATGCTGCAAGTTCCTGAGCGCCCTCC
CAACACTGAGGTGGGGGCTAGTCTAATCCCCATCCTAGAGGTGAAAACAGTGAAACTAGGACTCACAAGGCAAATTA
GCCTGTTCAGGGTCACCGAGGGTCCACTCTCATGGGAGAGTTTGCAGATGCCCAATCCGGCATTCTGCTGAGTGTCC
AGTGGCTTGTAAGTGGCCAGACACCCTTTGAGCTCAGCCTCAGCTGCTCAGGCACAGAACGTGCCTGGAGCTTGGAA
TTCAGGCCAGAAACCACCAGTGGACACCAGCATTCCACACTCACTGCACAGGCTGGGGCTCAAACCAAGGCCCAGGG
ACAGGAAGGGACAAGCCCCAGCCCCAGCCGGACTCCCAGCCCACACAAACCATCAGGGCTTGTTTCCTGCTCCATGG
AAGCCTCAGACATGTTTCATAACCTCCTGGAGCCTCCGTTTCCTTATCTTTCCAATGTAATGATGCCCATGTGCAGT
GGCTCACGCCTGTAATCCCAAGCACTTTAGGAGGCCGAGGTGGGTGGATCACTGGAGCTCAGGAGTTTGAGGCCAGC
CTGGGCAACATGGCAAAACGCCATCTCTACTAAAAACACAAATATTACCCAGGCATAGTGGCACATGCCTATAGTCC
CAGCTACTCAGGAGGCTGAGGTGGGAGGATCACCTGAGCTTGGGAAGTTGAGCCTGCAGTGAGCCAAGATTGTCACA
CTGCACTCTAGCCTGGAGGACAGAGTAAGAAGACCCTGTAACAAAACAAAACATAACAAAACAAACAAACAAAAAAC
CCAACTAATGACAATAAAATAAACCCTCCCTCACAGGGTGGTTGTGAGGATAAAGCACCCAGAATGAAGAGTGTTGC
TGCCATGTGCAGAACTTAGAAAGTGCTCAACAGATGCCAGCCAAACAGACATGGACTCCCCTCAACACAGTCAACCC
AAGGTTGACTGTCACCAAACGCAAAAGACCACACTGTAAAGCTTTTAGAAATGTGGTCTAGTGGCCGGGCACTGTGG
CTCATGCCTGTAATCTCAGCACTTTGGAAGGCTGAGGCGGGCGGATCACAGGGTCAGGAGTTCGAGACCAGCCTGAC
CACCTGACCAACGTGGTAAAACCCCGTCTCTACTAAAGATTCAAAAAATTAGCCGGGTGTAGTGCTACGTGCCTGTA
ATCCCAGCTGCTCGGGAGGCTGAGGCAGGAGAATCGCTTGAACCCAGGAGGCGGAGGTACAGTGAGCTGAGATCGCG
CCATTGCACTCCAGCCTGGGAGACAGAGAGAGACTCCGTCTCAAAAAAAAAAAAAAAAAAAAAGTTAGCCGGGTGGT
AGTGGCATGTACCTGTAATCCCAGCTACTTGGGAGGCTGAGGTAGGAGAATCGCTTGAGCCTGGGAGGTAGAGGGTT
GCGGTGAGCCAAGATGGCGCCACTGCACTCCAATCTGGGCGAGACACTGAGACCCTGTCTCAAAAAAAAAAAAAAAA
TGTGGTCTAGGAGACTCTCTTCACTTTGAGATAAAATTTGCATCACGTAAAGATAACCATTTTAACGAGAGCAAGTC
AACGGCATTCAGCACATTCAGAGTGTTGTGCAACAACCACTTCTCCCTGGTTCCAGGACATTTTCATCGCCTCAGAT
GGAAACGCCCTCCTCACGGAGGCATCTCTCCCGGCCTTTGTCCTCCCCGGCCCTGACAACCACTAATCTACTTTCTG
CTGGGATTTGCCCATTCTGGATGTTTCCTAAAAATGGCTTATCTAAGCCCCACAGTTTCATGCAGCACGTAGCCTCT
GGTGTGTGACGTCCTTCACTTGGTGTAATGGTTCGAGGCTTGTCCATGTCGTAGCCTGGGTCAGAACTTCATTTTCA
TGGCTGAATAATATCTCACGGTGTGGAAATATCACAGTTTGCTTATCTGTTCATCCAGTGATGGACATTTGGGTTGT
TTCTACCTTTTGGCTATTGGGAATGGAAGGGATAACATTTTTTAATTGGATTTTTAAAGTCACTAGTTTGACTGCAT
TAAAATTACAAACTTTTGTTTAACGAGAATATCACTAAGATACAGAGTTGGGGAGATCTAACACATAAAAGTGACAA
AGGAATTATATCCAGAATATTTTTGAAATTTCTACAAATCAGTGACTGGCAACACAGTGGGAAAGTGGCCAAGACTA
AAATACTTTAATAAAGAGGAAACCGAAATGGCCAGTAAATATGGGCTCAACCTCACTAATTATCAGGAAAATGTAAA
TTAAGACCACAAGAGAAACCACTACACACTCACCAAAAATCACACACCCAATAAAAAGGTAATTTTTTTTTTTTTTT
GAGATGAAGTCTCACTCTATTGCCCAGGCTGGAGTACAATGGCGCGATCTTGGCTCACTGCAACCTCCGCCTCCTGG
GTTCAAGCGATTCTCCTGCCTCAGCCTCCTGAGTACCTGGGATTACAGGCGCACACCACCACACCCAGCTAATTTTG
CATTTTTAAGTAGAGACGGGGTTTCACCATGTGGGCAAGGCTAGTCTCGAACTCCTGACCTCGTGATCTGCCCGCCT
TGGCCTCCCAAAGTGCTGAGATTACAGGCATCAGCCACTGTGCCCGGCCTAAAAAAGGCTAAAATTTAAGAAGACCA
GGAGTTTGACTGCTATGGTTGGAATGTTTGTCTCCTCTAAAACTCTTGTTGAAACTTAATCCCCAGTGTGGCAGCGT
TGAGAGGTGGGGCCTTTGGGGTAAGGAGGTTGGATCATGAGGGTCCTCCCCCAAGGAATGGATTAATGAGTTGTCAT
GGGAGTGTGGCTGGTGGCTTTATAAGAAGAGAGACCTGGCCGGGCACGGTGGCTGACACCTGTAATCCCAGCACTTT
GTGAGGCCGAGATGGGCGGATCACAAGGTCAGGGGATCGAGACCATCCTGGCTAACACAGTGAAACCCTGTCTCTAC
TAAAAAAAAAATGCAAAAAAATTAGCCGGGCGTGGTGGCGGGCACCTGTAGTCCCAGCTACTAGGAAGGCTGAGGCA
GGAGAATGGCGTGAACCTGGGAGGCGGAGCTTGCAGTGAGCCGAGATCGCGCCACTGCCCTCCAGCCTGGGCGACAG
AGCAAGACTCTGTCTCAAAAAAAAAAAGAAGAGAGATCTGAGGTGGCACACAAGCATGCTCAGCCCACACGACCTGC
GATTAATACTCTGTGCCACTTTGGGACTCTGCACGAGTCCCCACTGGGCTCGAAACTTCTCAGCCTCCGTAACTATA
GGAAATAAATTCCTTTTAAAATAAATTCCACAGTCTCAGGTATTCTATTATAAGCAACAGAAAATGGAGTACTACAC
CGATCATATCAAATGTTTAGAAGGATTTGGAGCAAGGAGAATGCTCGCACACCACTAGGGAAAACATAAGTTGGTTA
ACCACTGTGAAAAAGTTTGGCATTCTTTACTAAAGTTGAAAATCTATATGCCCTATGACCCAGCAACTTTACTCCTA
GGTATGTATGTACAAAATAGAATTTCAGGCATGTGGGTACCAGGTGACATGTAAAGGAATGTTTATTGCAGCATTAT
TCATAATAGCCAAGAACTAAACAACACAAAGTTCCAGCCCCAGTACAATGAATAAACTGTGGTATATTCCTACAAGG
AAATATTAATAGATACAGCAATGAAAATGAACACATATAACATGGCTGGTAAATCTGACATGAGAGAGTGAAAGAAG
ATGGACATTCAGTGTGCAGACAGTTGGATTAAAAATATTTTTTTAAAGGCCAGGCTTGGTGGCTCACATCTATAATC
CTAGCACTTACAGAGGCCAAGGCGGGCAGATCACCTGAGGTCAGGAGTTCAGGACCAGCCTGGCTAACACAGTGAAA
CCCCATCTCTACTAGAAAATACAAAAATTAGCCAGGTGTGGTGGTGCATGCCTGTAGTCCCAACTACTCGGGAGGCT
GAGGCAGGAGAATCACTTGAACCTAGGAGGCGGAGGTTGCAGTGAGCCAAGATCGCATCACTGTACTCCATCCTGGG
TGACAGAGCAAGACTGCGTCTCGAAAATAAATAGATAAATAAATAAATAACCAACAGGCCGGGAGCAGTGGCTCATG
CCTGTAATCCCAGCACTTTGGGAGGCTGAGGTGGGCAGATCACGAGGTCAGGAGATCAAGACCATCCTGGCTAACAC
AGTGAAACCCTGTCTCTACTGAAAATACAAAAAAATTAGCCGGGCATGGTGGCGGGCGCCTGTAGTCCCAGCTACTC
AGGAGGCTGAGGCAGGAGAATGGCATGAACCCGGGAGGTGGAGCTTGCAGTGAGCCGAGATCATGCCACTGCACTCC
AGCCTGAGCGACAGAGCGAGACTCCATCTCAAAAAAATAATAATTAAAAATAAATAAATTAAATAAATAAATAACAG
ATTGCATAAAGTGGCTCATGCCTGTAATCCAAGCACTTTGGGAGGCCAAGGCAGAAGGATCACTTGAGCCCAGGAGT
TCAGGACAAGCCTGAGCAACATGGTGAAACCCCACCTCTACAAAAAAAAAAAAAAAATTAGCTGGGCATGGTGGCAT
GTGCCTGTGATCCCAGCTACTTGGGAGGCTGAGGCAGGAGGATCACTTAAGCCTGGGAGGTCGAGGCTGCAATGAGC
TATGATCGTACCACTGCACTCCAGCCTGGGCAATAGAGCAAGACCCTGTCTCAAAACAAATAAACAAAAGCCAGACA
GACACAAATGAGAGCATTCTGTATCGTTTCATTTCTATGAAGGTGAAAAGCAGGCAAAAACAACCAAAGTGCTTGCA
GATGCATATCTGAGTAGTTAAAAACTTACTGAAAAGCAGGCCTGGCTCACGCCTTTAATCCCAGCACTTTGGGAAGC
GGGCGGATCACGAGGTCAGGAGATCGAGACCATCCTGGCTAACACGGTGAAACCCCGTCTCTACTAAAAATATAAAA
AATTAGCCAGGTATGGTGGCTAGTGCCTGTGGTCCCAGCTACTCGAGAGGCTGAGGCAGGAGAATGGCATGAATCCG
GGAGGTGGAGCTTGCAGTGAGCTAAGATCGTGCAACTGCACTCCAGCCTGGGCAGCAGAGCGAGACTCCCTCTCAAA
AAAAAAAAAACTTACTGAAAAGCAAGAAGTCAGGTGGAGGTTACCTTTGGGGAGGATTGGGGTGCTGTCCGCTTTCT
AATAATTCGTTAAACTATAGTCTACATCTTGTGCTATATTTCACAATGGAAAAACAGAAAAGAGCTCCTGCCCATAA
CGCTGCTTTGCAGGTTTGGAAATTTCAGATTCAATTCCTCTCCTTGCGGGGGCCAAGGATGGGAAGAGCAGGTGGTT
CCAGTAGGGAAAGAGGAGGCCCTGGGGCCTCAAAATGGCTAAGGACCATTCCTCAGCGTGGGTGGCACCTACCCTGG
AAACAGGACTCTACTTCCTCCTCTGTTAGGGGGCAGAGCAGCCCTGCAGTGCCTTCTGGGCACAGGTCCTCACTCTG
CAGCTGGAGGAATTCTCCCAGGCACTGAGAGCCCTTCACGGCCCAAATGCCCCGTGCGCTCGGCCTCTGGACTTGCC
TTCCCTGCTCTGTATATCTCCCTCCGCCTGACCCTCAGCCTCCTCCATCACTCACTGTCTTCTCTGCCAGTCTATTC
ATCTGTCTCTGTCCCTCTCTCTGCCACCTTCTCTCCTATTGAGAAGCCGAAACCTCAGGCACAGACCCACATCCCCT
CCTCATGGGCCCATGTGCCCAAGGTGCCCCTAGGTGCCAGGCTGAGATGAACCAGGAGTGTCCTTCTGAACCCAGCA
ACAGCGAAGGGTGACCAGGGAGGGCCAGTTCATCTCGGTCTGAAAGAAGCCCCAGATGAGCAAAGGATACACTGGCC
TCCTGCGGTCAGCAGCACTTCCCAGGACAGTGAGCAAGACAGGGGTAAGGCCAGAGTGGGTGGGCACACCCATGGGA
GAGAGGAGCCGCTGTGAAATGTGCACGAGGAACAGACCAGCAAGGAGGATCCACGCAGTGCTAGAAGGGAGTTCCTG
GAAGCCTGGTGGAGAGCCCCTCCCATCTGCTAAGCCCGGAGGGCATCAAAGGCTGCTGCTGCCCTCAACCCCTGACA
ATCTCATCATCTCATATCTCAGGCATGGAAGAATGAGGGCCATTACACGAGTAAAACATCAAGTACACTCCAGCCTG
GATGACAGGGCCAGGCTCCATCTCAAAAAAAAATGCCTGTGGTCAAAGCTCTCCTGACAGGGGAAAACAAAACAAAA
CAAACTTCTCCTTAAAGAAAACATTTGCCTTTGACTGCATCATAATTCCAGCAGGATTTTGTGCAGATAACTCTTTG
GCTAACTCTAAAATTAATACAGAAAGGTAAAGAAATTAGAATAGCCAAAGAAATTTTGAAAAGGAAGAATAAAGCGA
GAGGAATCACATTCCTCAATTTTTAACAGCTCTATTGAGATAAAATTCACATACCATACGGTTCACCCATTTAAAGT
GTATAATTCAGGCCGGGCGCGGTGGCTCACGCCTGTAATCCCAGCACTTTGGGAGGCTGAAGCGGGCAGATCACCTG
AGGTCGGGAATTCGAGACCAGTCTGACCAACATGGAGAAACCCCGTCTCTACTAAAAATACAAAATTAGCCAGGCGT
GGTGGCTCATGCCTGTACTCCCAGCTACTCGGAAGACTGAGGCAAGAGAATTGCTTGAACCCGGGAGACGGAGGTTG
CCATGAGCCGAGATCGCGCCACCACACCCAGCTGCCATTTTTTAATTGATTACTTGTCTATTTATTACTGAGTTGTA
AGATATTTTGGGCCAAGCACGGTGGCTAACGCCTGTAATCCCAGCACTTTAGGAGGCTATGGTGGGCAAATCACTTG
AGGTCAGGAGTTCGAGACCAGGCTGGCCAACATGGCAAAACACCATCTCTACTAAAAATACAAAAAAATTAGCCAGG
TGTGGCCAGGCGTGGTGACTCACGCCTGTAATCCCAGCACTTTGGGAGGCCAAGGCGGGTGGATCACCTGAGGTCGG
GGGCTCAAGACCAGCCTGACCAACATGGAGAAACCCCGACTCCGCTAAAAATACAAAATTAGCCGGGTGTGGTGGTG
CATGCCTGTAATCCCAGCTACTCACGAAGCTGAGGCAGGAGAATGGCTTGAGCCCAGGAGGCAGAGGTTGTGGTGAG
CTGAGATCATGCCATTGTACTCCAGCCTGGGCGACAAGAGCGAAATTCTGTCACAAAAAAAAAAAAACCATTAGCCA
GCCATGGTGATGCACACCCGTGGTCCCAGCTACTCAGGAGGCTGAGGTATGAGAATTGCTTGAACCCAGGAGGCAGA
GGTTGCAGCGAGCCAGGATTACGCCGCTGCACTCCAGTCTGGGTGACAGAGCAAGACTCTGTCTAAAAAAAAAACAA
AAACAAAAAAGATATTTTGTATGTGTTTGGATAACTTCCCTATCAGATATATGATTTGCAAATATGTTTCTCTCATT
CTGTGAGACATCATTCAATTTTAAGACATCACAGAGCTATGTTAATCAAGGCACTGTGGCTGTGGTAAAGGATAGAC
ACACAGAACAGAACAGAGAGCCCAGAAATGGACCCGCAAACCTATGCCCCATTCATTTTTTACAAATAAGTGCGAGA
AGCCAACTGAATAGAAAGCGTATAGCTTTTTCAAAAAACAGTGCTGGAACAATTGGACATCTGTAGGCAAAAAAACA
AACAAGCAAACAGAAGAATCTGGACCTGCCCTTCACACCTCAGACAAAAGTCATCTCAAAATGGATTGTAGATCTCA
ATATAAACATAAACTATACAACTTTAGAAGAAAATATAGGTGAAACTCTTTGTGTTCTGTGGTTAGGCAGACAGTTC
CTAGGCATGGCACTAAGTAAGATTCATTTAAAATTTTTTGACAAATTGGACTTTATTAAAACTTTTGCTCTACAAAA
GACAATATTAAGAGAATGAACTAACAAGCTACAAACTAAGAGAAAACATTTGCAAATTGCATATCTGACAAGGGATT
GCTTCCAGACGATACACAGAATTCTAAAAATTCATCCTTAAGAGAATAAACCACCCAATTTTTAAATGGGCAAAACA
GGCCAGGCGTGGTGGTGCACGCCTGTAATCCTAGCACTTTGGGAGGCCGAGGCAGGCGGATCACAAGGTCAGGAGAT
TGAGACCATCCTAGCTAACACGGTGAAACCCTGTCTCTACTAAAAATACAAAAAATTAGCCAGGCATGGTGGCAGGT
GCCTGTAGTCCCAGCTACTCGGGAGGCTGAGGCAGGAGAATGGCGTGAACCTGGGAGGCGGAGCTTGCAGTGAGTGG
AGATCGCACCACTGCGCTCCAGCCTGGGCAACAGAGCGAGACTCCGTCTCAAAAAAAAGACAAAATACTTGAAAAGA
TATTGGCTAGGCGCGCTGGCTCATGCCTGTAATCCCAGCACTTTGGGAGGCCAAGGCGGGTGGATCACAAGGTCAGG
AGTTCAAGCAGCCTGGCCAAGATGGTGAAACCCCGTCTCTACTAAAAAAAAAAAAAAAAAAAAAAAAAAAATTGGCC
GGGCACAGTGGCTCATGCCTGTAATCCCAGCACTTTGGGAGGCTGAGGCAGGTGGATCAGGAGTCAGGAGATCGAGA
CCATCCTGGCCAACATGGTGAAACCCCATCTCTATGAAAATACAAAAATTAGCCAGAGATGATGCCGGGTGCCTGTA
ATCCCAGCTACTCATGAGGCTGAGGCAGAAGAATCACTTGAACCAGGGAGTCAGAGGTTGCAGTGAGCTGAGATCGC
ACCACTGCACTCCACCCTGGGCGACAAATCGAGATTCCATCTCAAAAAAAGAAAAAAAAATTAAAAGGAATATTTGC
CTCATTATGTTACAATAACTAATATGGAAAGCAATATTGCAATGCCTATTAGCACATGACATTAGGTGAATTCTCCT
TTGTCCCCGGACCTGCTGCCTCCTCCTGCTTGTCAGGGGACAGATCCAGTACATCTCCCCTCAGCGCTGGGTGGACC
TAACCCTTGCTTTCTTGGAGGAAACCCAGGAATCCAGAGACAAAGTGGAAGGGTACTGGCATGTGGTTGGGCAGGGC
TGCCTGAGGTCGGTGTCAGCCGACCGTGGGGCTTGGTCCCAGGAGGCTGCTTACTGGGCCCTGCTCCTCTGGTTTCC
CCCAAGTCGTGATTCTGAAATGAATAAGGACGGTGCAGAACTGGACTACAAATGCAGGAGTGACTTCCTGGGAGGGT
GGGGCCCCTATCTCTCCTAGACTCTGTGGTCAGACTCTGGCCAACACCCCCTGTAAGGCCACAGGAGAGGAACAGGA
GTGATAGCCCCCAAACCCCAGTCCCACCAGGCCCTGAGGGCCCCTTTGTCACTGGATCTGATAAGAAACACCACCCC
TGCAGCCCCCTCCCCTCACCTGACCAATGGCCACAGCCTGGCTGGGCCCAGCTCCCTGTATATAAGGGGACCCTGGG
GGCTGAGCACTACCAAGGCCAGTCCTGAGCAGGCCCAACTCCAGTGCAGCTGCCCACCCTGCCGCCATGTCTCTGAC
CAAGACTGAGAGGACCATCATTGTGTCCATGTGGGCCAAGATCTCCACGCAGGCCGACACCATCGGCACCGAGACTC
TGGAGAGGTGAGTGTCAGACGGGACTGCCAGAGGGACTGGGTGGGAGGCCAGGTATGTGAGTGGGGACAGTGGGGAG
GGGGCGGTGGGGAGGGGACAGTGGGGAGGGGACCATGGAGAGGAGACAGTGGGGAGGGCACTGTGGGGAGAGGACAG
TGAGGAGGGGACCTTGGGGAGGGGACAGTGAGGAGGGAACCGTGGAGAGGGGACAGTGAGGAAGGGACAGTGAGGAC
AGATAGCGTTCCCTCTCAGTGAGGAGAGCAGGGTAAGGAGGGAACGATTAGGAGTTGCACAACCATCTGGGCTCGCT
GAGACCTGGGCAGGCACAGGCCCAGGTTCTGACAAGCAGAGGGTGAAAGGTTTCGTTCTAGGCCTGAAGGGCCTTAC
AGGGCAGCCAGGGCACTACAGCCTCTAAAGTCCCAGCATCTGGGATCAGGGCACTGTCCCAGCTTCAAATTCCCAGC
ATCTGATCCCCTGGGAGGGGCCAGGGAGCTTTTCCTTCCCTGGAACGCTGCTGGGAGGTCATGAGCCTGCAGAAGGG
GTGGCGGGCAACCCAGTCTGGGGCTGGGAGGGAGGTCCTGTGGCCAGAGGAGACGGTGGAGGGGCTGGGGGCACCAG
GCGTGCTGGAGGCGGAGGGCGGGAGATTTGGGGACCAGGCTGCACAGAACCCGTCGGAAGCAGGGCGATCAGCCGGG
AGCTGCAGAGGCCTGGGGGGCCTCTAGCCCAGGGCAGCCTGGGAGGGGCAGCTGCCTGGGCACCCGGGCCCCGCGAG
GAGGGGCTGGGGCCTGCTGCGGGGTCGCAGATGTGTCCCGGTGCTCGGAGAGGGCCGCAGGGCGCGTGGGCCGTGGC
GGGAGGCCGCGCTGCTGGGAGCTCACGGCCCCCGCCCCCCGTCCCAGGCTCTTCCTCAGCCACCCGCAGACCAAGAC
CTACTTCCCGCACTTCGACCTGCACCCGGGGTCCGCGCAGTTGCGCGCGCACGGCTCCAAGGTGGTGGCCGCCGTGG
GCGACGCGGTGAAGAGCATCGACGACATCGGCGGCGCCCTGTCCAAGCTGAGCGAGCTGCACGCCTACATCCTGCGC
GTGGACCCGGTCAACTTCAAGGTGCGCGGGGCGCGGTGCGGGCGGGGGGGGACGGGGCGGGGCGCGGTGCGGGCGGG
GCGGGGCGGGGCGGGGCGGGGAGGGGCGGGGAGGGGCGGGGTCGCGGGGCGGATGCGGGGGTCGCCGGGCGGGGCCC
GGGCTAGGCCCCGCCCCCTCACTGAGCCGCCCCCGCCCCCAGCTCCTGTCCCACTGCCTGCTGGTCACCCTGGCCGC
GCGCTTCCCCGCCGACTTCACGGCCGAGGCCCACGCCGCCTGGGACAAGTTCCTATCGGTCGTATCCTCTGTCCTGA
CCGAGAAGTACCGCTGAGCGCCGCCTCCGGGACCCCCAGGACAGGCTGCGGCCCCTCCCCCGTCCTGGAGGTTCCCC
AGCCCCACTTACCGCGTAATGCGCCAATAAACCAATGAACGAAGCAGCGTCCACCTGGTCTCTGTTGTCCGTGGGCG
GCGGGCGCTTGGGGAGGCGGAGCGGGAGGAGGGCGCCCCGGCTGTCTCGGGGCCACTGCTGGGCCGCAGGGATCCTT
GCACCGACCCCAGGGTCTCTAAGAGGCAGAGGGATGTGCAGCTCCCGGGGGGGGAGCGGGGGTCACTCGGGACCCAG
GCGTGGTGGAGAAGGGGTGCAGTTAGGCCTTTGCGGAGGGGGGAGCAGTGCTGGCGCCCACCCGCCGCGGCTCTCCC
TGGGACCTCCGTGGTCTTCCTTCTTTATTTCTCCCGAATGTGTACTATTTCCTGATTTCAGAACGATCAGGACGAAG
AGGGGAGGGATGGGCGTCTGCGCTCACTCATTCCTTCTTCCATTCCTCAATGAAACATTTACTGGGCATAAGACAGC
CTAGGCATGTTTCTAGGCTATGGATACCGCAGCTGAAATAAAGAAAGCCCTCTGCCCCGTGGGGCTGACAATCTAGT
GGGGGATACAGACGTGATGAAGACAGTCAGATCACAGTTCACAGAAATGAGACAGGAAAAGAGGCTGAGCCTCACTC
ATAAGAGAAACGCAAGTTAAACTACACAAAAATAAAAAACCTCACTGAGATCCATGTCTCACCTCCCTGATAGGCAA
AAATCCAAGAGTTTGATCAGACTGCAGGCGCCCCTCCTCCACTGGGCACCCCTCATCCAGGGCAGAGGGAACCAGCC
CGGGGCGCAAGTCCACCGGGGCATCTCATTTGCTAAAGACCTGAAAACCCAGGTGTCCATCATCAGGACTAACTGGA
AAAACCAAGGGTATCCGCACCATGGAGAGCTCGACTGAAAAAAAAAAATGAGGATAATTGGATAATTTCTTTTTTTT
TTTTTTTTTTTTTCAGACGGAGTCTCGCTCTGTCGCCCAGGCTGGAGTGCAGTGGTGCGATCCCGGCTCACTGCAAG
CTCCGCCTCCTGGTTTCAAGCGATTCTCCTGCCTCAGCCTCCCGAGTAGCTGGGTCTACAGGCGCCCGCCACCACGG
CTGGCTAATTTTTTGTATTTTTAGTAGAGACGGGGTTTCACCGTGTTAGCCAGGATGGTCTCGATCTCCTGACCTCG
TGATCCACCCGCCTCGGCCTCCCAAAGTGCTGGGATTACAGGTGTGAGCCACCGCGCCCGACCTAAAATGAGGATAA
TTTCTAATAATGAAAATAAAGAGGTTAGAATGGTGTGTATACAATGGTGGAACAGAGGAGAAACACGAATATGTGTG
TGCACATATATGTGAGCTTATGCATAACTATGTATGAGGCTGCGTGTGGACATGTGTGTTTGTGCACAACCATGTAT
GTGCCCGCATGTGCTTATTTCTGCAAAAATAAACCATGGCAGGACAAACCGGAAATGAATACAAATAATAAGGTGGG
TGGGGATGGAGGGGAAGGTGGAAGGAAGCTCCTGCAAGTCTGACTCTCTACATAGTTTTGACCTTTGATTTGTGTAA
ATATTTTACATTATCAAAAATAAATTCAGGCTGGGCATGGTGGCTCATACCTGTAGTCCTAGCACTTTGGGAGTCCA
AGGGGAGAGGATTGCTTGAGGCCAGGAGTTGAAGGCCACCCTGGCCAACATAGAGAGACCCTGTCTTTAAAAAAAAT
TACAAAATTAAGGCCGGGCGCGGTGGCTCACGCCTGTAATCCCAGCACTGTGGGAGGCCGAGGTGGGCGGATCACGA
GGTCAGGAGATTGAGACCGTCCTGGCTAACACGGTGAAACCCCGTCTCTACTAAAAAGTAGAAGAAATTAGCCGGGT
GTGGTGGCGGGTGCCTGTAGTCCCAGCTACTTGGGAGGCTGAGGCAGGAGAATGGTGTGAACCCGGGAGGCGGAGCT
TGCAGTGAGCCAGGTTCAAGCCACTGCCCTTCAGCCTAGGTGATAGAGTGAGACTCCTTCTCAAAAAAAAAAAAAAA
ATTACAAAATTAATAAGATTAAAATAAAAAGAGGGGCCTTGCCAGTGGCTCAAGCCTCTAATCCTACCACTTGGGAG
GCCAAGGCTGGAGGATCCCTTGATGCCAAGAGTCGGAGGCCAGCCTAGGTAACACAGCAGGACCTCGTCTCAAAAAG
ATTAAAAAATTAACTGGGCATGGTAGCCTCCAAATTGGGGGTTAGCCTGGGAGGTTTGCCCAGGAAGGAATTCAAGG
GCAAGCTGGTGGTGTTACACAGCAACTCTGATTGATATCGAAGCCACAGCAGACAGCAGGAGCAGAACACTGCTCCT
TACAGAGCAGGGGTACCCCATAGGCTGTGTGCACAGGAGAGCAACTCAGAGGCACTGCTGCACTCATCTTTATACCC
ACTTTTCATTATATGCAAATTAAGGGAAAGTTATGCACAAATTTCTAGGATGAGTGTGGTAACTTCTGGGTGGTCCA
GTCACTGCCATGGAAAGGGATGGTAAACTCCCATGGCACACTGGTGGGTGTGTCTTATGGAAAGCTGCTTCTGCCCT
ACTTGTTTTAGCTGGTCCTCAGTTTGGTCCGGTGTCCGAGCCCAACATCCGGAGTACATGCAGAGTCCCACCTCCTA
CGTCACACCTGCAGTTCCAGCTACTCAGGAGGCTGAGGCTGGAGGATTGCTGGAGCCCAGATGTTGAAGGCTACAGT
GAGCTATGATTGTGCCACCGCACTTCAGCCTGAGCAACACAGCAATACTCTCTCTCTAAAAAAGCAAAGCACACAAA
CAAAAAGAGTGACTGGGTGCAGTGGCTCACACTTGGAATCTTAGCACTTTGGGAGGCCAAGGTGGGATGGTCACTTG
AGCCTGGGAGTTCAAGACCAGCCTAGGCAACATAGCAAGACTTTATCTCTACTAAAATATATATATATTTTTTAATT
AGCTGGACATGGTGGTGCACCTGCAGTCCCAGCTACTTGGGAGGCTGAGTTGGGGGTGGAGGGGAGTATCACTTGAG
CCCAGAAGTTCCAGGCTGTAGTAAGCTATGATTGCACCACTGCACTCCAGCCTGGGCAACAGAGAGAGACCTTATCT
ATATTTAAAAAAAAAAAAAAAAAGAGAGAGAAAATTGAAAACTCCTAATTGAAAACCCCCAAATTGAAAACTAACTT
AAATAAATGAGCCAATGTAAGAATGTGGTGATATAATAATCAGAAAAAAGGATTGTTCCAGGTGACCTCTGAACACA
GAACCTCGGCTATGACCGAAAGAACTCCAAAGACACTCTAACACTCCGTGGTTTATTGTTCCTCATAACATATATAA
AATAATTTCATAAGCTTTTATTTTGAAACATATTCAGATTATGAAGAAATAAAAACACCCTGCAAGAATAAGACAAA
GATGGAGAAGGAAGGATGACTGCTGGTGGGTTTGGGGCTTTTGGAGGGTGATGGAAACCTTCTAAAATTGATTATGG
TGATGGTCGCACAATTATGTGAACACATTAAAAATTATTGAAATGGGCCGGGGGTGGTGGCTCACCCCTGTAATCCC
AGCACTTTGGGAGGCCAACGCGGGCAGATTACCTGAGCTCAGGAGTTCCAGACTAACCTGGCCAACATGGTGAAACC
CCCGTCCCTACTAAAAATGCAAAAATTAGCCACGCATGGTGGCACATGCCTGTAATCCCAGCTACTGGGGAGGCTGA
GGCAGGAGAATTGCTTGAACCCAGGAGACAGAGGTTGCAGTGAGCCGAGATTGTGCCACTGAACTCCAGCTTGGCCG
ACAGAGTGAGACTCTGTCTCAAAAAAAAAAAAAATTATTGAAATGTACACATTAAGTGGGTGAATTTTATCTCAATA
AAACTGTTAAATAAAATAACAAGAATATGAAAAACTCTTGAATACTACTCATCCAGACTCTCCAGCTGTTAACATTC
TACCACATCGGCTTGCTCTCTCTTGCCCCCACTTGCTCTTTCTCTCGGAGCCCTTGGAGAGGGGTATGCAAATATCC
GTACTCTAAATATCCTCCATATACTGTGTATTTCCTAAAATCAACAAGGACATTAGGCTGCACAGCCAGAGAACAAC
CATCAAAATCAGGTTAATATTGATCCAAATCCATCTATCAACAGAAGCAACATCAAGTTCAAGACCCTTTTGAAAGC
AATGATACCAGCCATTTACTCCATCCCTAAAGGACTGAGGGTGCTGCGAATTTAACCGTATCAATGCAGTCTTTTTG
ATGTTATTTACTGAAGGAAATGGATGTTCTTTAAAATATGTATTTATTTATTTTTCTTTTTTGAGACGGAATCTTGT
TCTGTCGCCCAGGCTGGAGGGCAGTGGGACAATCTTGGTTCACTGCAACCTCTGCCTCCTGGGTTCAAGAGGTTCTC
CTGCCTCAGCCTCCCGAGTAGCTGGGATTACAGGCGCGAACCACCACGCCCGGTTAATTTTGGTATTTTTAGTAGAG
GCGGGGTTTTACCATGTTGGCCAGGCTGGTCTCAAACTCCTGACATGGTAGCCTGTAATCCCAGCTACTCGGGAGGC
TGAGGCAGGAGAATCGCTTGAACCCAGGAGGTGGGGTTGCAGTGAGCCAAGATCGTGCCATTGCACTCCAGCCTGGG
AGACAGAGCGAGACTCCATCAAAAAAAAAAAAAAAAAAAATTCCTGAAGCTCCTCTTGAGCTTACATTCTAGTGGAC
TGTAAACAGAAACATTTTTTTTTCCTGTGGATAAAGAAAAGCAGGGCAAGTAGGGGCTTAGACAGAGGAGGGGAGGA
TTCAGATTTTAAATGGGTTGGCCACTGTAGGTCTATTAACGTGGTGACATTTGAGGGAGTGGCAATACTAGGGAAGG
GGCTTCAGGGGAGTGGCCAGGAGCTAGGGATAGAGGGAGGGAGGACAGGAGGCCTTGTCTGTCTTTTCCTCCATATG
TAAGTTTCAGGAGTGAGTGGGGGGTGTCGAGGGTGCTGTGCTCTCCGGCCTGAGCCTCAGGAAGGAAGGGCAGTAGT
CAGGGATGCCAGGGAAGGACAGTGGAGTAGGCTTTGTGGGGAACTTCACGGTTCCATTGTTGAGATGATTTGCTGGA
GACACACAGATGAGGACATCAAATACATCCCTGGATCAGGCCCTGGGGCCTGAGTCCGGAAGAGAGGTCTGTATGGA
CACACCCATCAATGGGAGCACCAGGACACAGATGGAGGCTAATGTCATGTTGTAGACAGGATGGGTGCTGAGCTGCC
ACACCCACATTATTAGAAAATAACAGCACAGGCTTGGGGTGGAGGCGGGACACAAGACTAGCCAGAAGGAGAAAGAA
AGGTGAAAAGCTGTTGGTGCAAGGAAGCTCTTGGTATTTCCAATGGCTTGGGCACAGGCTGTGAGGGTGCCTGGGAC
GGCTTGTGGGGCACAGGCTGCAAGAGGTGCCCAGGACGGCTTGTGGGGCACAGGTTGTGAGAGGTGCCCTGGACGGC
TTGTGGGGCACAGGCTGTGAGAGGTGCCCAGGACGGCTTGTGGGGCACAGGCTGTGAGGGTGCCCGGGACGGCTTGT
GGGGCACAGGTTGTGAGAGGTGCCCGGGACGGCTTGTGGGGCACAGGTTTCAGAGGTGCCCGGGACGGCTTGTGGGG
CACAGGTTGTGAGAGGTGCCCGGGACGGCTTGTGGGACACAGGTTGTGAGAGGTGCCTGGGACGGCTTGTGGGGCAC
AGGCTGTGAGGGTGCCTGGGACGGCTTGTGGGGCACAGGTTGTGAGAGGTGCCCGGGTCGGCTTGTGGGGCACAGGT
TGTGAGAGGTGCCCGGGACGGCTTGTGGGGCACAGGTTGTGAGACGTGCCCGGGACGGCTTGTGGGGCACAGGCTGT
GAGGGTGCCCGGGTCGGCTTGTGGGGCACAGGCTGCAAGAGGTGCCCGGGACGGCTTGTGGGGCACAGGCTGTGAGG
GTGCCCGGGACGGCTTGTGGGGCACAGGCTGTGAGGGTGCCCGGGACAGCTCGTGGGGCACAGGTTGTGAGAGGTGC
CCGGGACGGCTTGTGGGGCACAGGCTGTGAGGGTGCCTGGGACGGCTTGTGGGGCACAGGTTGTGAGAGGTGCCCGG
GACGGCTTGTGGGGCACAGGTTGTGAGGATGCCCGGGATGGCTTGTGGGGCACAGGTTGTGAGAGGTGCCTGGGACG
GCTTGTGGGGCACAGGCTGTGAGGGTGCCCGGGACGGCTTGTGGGGCACAGGCTGTGAGAGGTGCCTGGGACGGCTT
GTGGGGCACAGGCTGTGAGGATGCCCGGGACGGCTTGTGGGGCACAGGTTGTGAGGGGTGCCCAGGACGGCTTGTGG
GGCACAGGCTGCAAGAGGTGCCCAGGACGGCTTGTGGGGCACAGGTTGTGAGAGGTGCCCGGGACGGCTTGTGGGGC
ACAGGCTGTGAGGGAGCCCGGCACGGCTTGCAGCTACAGGGAGAAAAGACTTGGTGCTGTGGGCCTGCCTTGGGGCT
GGTGGTACAGCCCTTATCTGCTGCCCTCAGGATCTCCCGGCCCCTCTCGTCCAGGCCCCTGCAACCCCATGCCCCAG
CCTCTGAGGACCAAAGGCGCCCCTGCTTGGGAAGAGGGGGCTCAGGGGAGTCGCCTGACCCGGTTCCAAGCCAGGCT
GATTTACCGTTGCTAACATCCTATCGCACGCATCCCTCTGCCTCATGCACCCAACCCCAAGGCCTGGTACACTGCAG
GCCCCAAGGTCCTGTGCGTCCTTTCAATACCCTCCTCACCTGCCTCACCTGCCCCCCCTACCCTGACTCTGGCTGGA
GACCCCCTCCAGGGAGTTTTCAAAACAAAGGGTGTCAGTCTCCTGTGGGATTCCCTCACCTCTGCAGCCTGCGGTCT
GAAAGCTGCCCCATGGTGTGTAGTGCTAAACTTCCAACTTACTCCAGGCCAGCGGTGACAGCCCGAGGGCAGGAAGG
GCACCCACACTGAGCCTCAAACAGCTAATTTTGCAACTGTAAGTCCATATAATTGTCTTGAAAAGTAATTTGTTTCA
AAAAGCTAAAAAACGAATACTCTTGAGTCTCCTTCTAGTAATTCCCCTTCTAGAGGTCTATCACCAGGAAAAGATCC
AAAGCACTGATATTCTTCATGGAGTTGTTTATAATAGAAAAAAACTAGAGCTTGTTCACAAAGGGGAGCTCTGCAGG
CTGAAGATGTTGCACCTGTCAGCGGGGATGGGGGCACGCTTGCTGACGCAGCAACGGAAAAGCATCAGTGTGTGAAG
ATGCATTTTCTCTCTTTCTATTATTATTATTTTTATTTTTATTTTTTCTGAGGCAGAACCTCGCTCTGTCACCCAGG
CTGGAGTGCAGTGATGCGACCTCATCACAACCACGAGCCACCATGTGCGGCCCCATGAGCAAGCCACCACGCCCAGC
CTTTTTTTCCCTTGTTTTAAAAAATCCTCTATTTAAAAAAGATGTGCATGGGCCGGGCACGGTGGTTCACGCTCATA
ATCCCAGCTCTTTCAGAGGCCGAGGCAGGCAGATCACCTGAGGTCAAGAGTTCGACACCAGCCTGGCCAACATGGTG
AAATTCCATCTGTACTAAAAATACAAAAATTAGCCAGGCCGTGGTGGTGTGTGCCTGTAATCCCAGCTACTCAGGAG
ACTGAAGCAGGAGAATCACTTGAACCCAGGAGGCAGAGGTTGCAGTGGGTCAAAATCATGCCACCACACTCCAGTCT
GGGAGACAGAGCAAGACTCCATCTCAGAAACAAACTAACAAACAAAATTTTTATATCTACCTATAATTCGTATAAAT
TTAAAATACATGCATAAAATCATACCCTTTGCAAGCACACGTACTAACTAAAAGGAATATATTCAGCACATAGAAAT
GGTTGTCTAACGGAGGAGGGGGGAGTTAATAAACAGAGAGGATAAAAAGAAATAAATCAGTAGAGCTGGAGGAGGGT
CTCCTCCAGGCTGCGATGAGAACATAGTGAGCAGAATTGCAGGCCTGCATGACCTCACCTTCTGTGAGGAGTCCGGC
CTCCCAAGACGCTTTCCTGCCTAGGTGCCCGGCTCAGAGTGTCCCCTACAAGGCTACTGGAGGAGAACCCCAGACCG
AGCCTCATTCAGGTGAGGGGGCTGCACACCGGAGGTGGGAGAGGTCTGTCCCTTCCCACCCTGTGACACTGGGTCCC
ACTTTCTCTCTAGGGGGTCTCGGTTTCCTCATTTGCAAACTGGAGCTCATAAGGTGGGCCAGAGAAGTTTCAGTGAA
GTGAGGAATGGATCGTCCCTCTGCCAGGGCCCATGTGCTCTAGGTCACCCTGTCATCACAGGGACAGGGAGGTCAAG
GACAGTCACTCCTGAGGCCAGTCCGGGCTGGGCTGACCACGTGGACTCTCATGCCCAGATTGGGGCCCCAATCTCCC
TGAAGCTGGGGCTCCAGCTGTGACTCAGGGGTGGGCAGAAGGGGAGACAGAAGCGATAGGTTCCTCAGCCCCCAGTC
CCACCTGAGGGCCCCTTTGTCACTGGATCTGATAAGAAACACCACCCCTGCAGCCCCCTCCCCTCACCTGACCAATG
GCCACAGCCTGGCTGGGCCCAGCTCCCTGTATATAAGGGGACCCTGGGGGCTGAGCACTACCAAGGCCAGTCCTGAG
CAGGCCCAACTCCAGTGCAGCCGCCCACCCTGCCGCCATGTCTCTGACCAAGACTTAGGGGACCATCATTGTGTCCA
TGTGGGCCAAGATCTCCACGCAGGCCGACACCATCGGCACCGAGACTCTGGAGAGGTGAGTGTCAGATGGGACTGCC
AGAGGGACTGGGTGGGAGGCCAGGTATGTGAGTGGGGACAGTGGGGAGCGGGCAGTGGGGAGGGGACCGTGGGGAGG
GGACAGTGAGTAGGAGACAGTGGGGAGAGGACAGTGGAGAGGGGACAGTGAGGAGGGGACCATGGGAAGGGGACCGT
GGAGTGGGGACAGTGAGGAGGGGACCATAGGGAGGGGACAGTGGGGAGGGGACAGTGAGGAGGGGACCGTGGGGAGG
GGACAGTGAGGAGGGGACCGTGGGGAGGAGACAGTGAGGAGGGGACCGTAGGGAGGGGACAGTGAGGAGGGGACCGT
GGGGAGGGGACAGTGAGGAGGGGACCGTGGGGAGGGGACAGTGAGGAGGGGACCGTGGGAAGGAGACAGTGAGGAGG
GGACCTTGGGGAGGGGACAGTGAGGAGGGGACCATGGGGAGGGGACAGTGAGGAGGGGACAATGGAGAGGGGACAGT
GAGGAGGGGACTGTGGGGAGAGGACAGTGAGGAGGGGACCATGGGGAGGGCACAGTGGGGAGGGGAGAGTGAGGAAG
GGACAGTGAGGAGGGGACTGTGGGGAGGGGACAGTGGAGACAGATAGCCTTCCCTCTCAGTGAGGAGGGCAGGGTAA
GGAGGGAACGATTAGGAGTTGCACAACCATCTGGGCTCGCTGAGACCTGGGCAGGCACAGGCCCAGGTTCTGACAAG
CAGAGGGTGAAAGGTTTCGTTCTAGGCCTGAAGGGCCTTACAGGGCAGCCAGGGCACTACAGCCTCTAAAGTCCCAG
CATCTGGGATCAGGGCACTGTCCCAGCTTCAAATTCCCAGCATCTGATCCCCTGGGAGGGGCCAGGGAGCTTTTCCT
TCCCTGGAACGCTGCTGGGAGGTCATGAGCCTGCAGAAGGGGTGGCGGGCAACCCAGTCTGGGGCTGGGAGGGAGGT
CCTGTGGCCAGAGGAGACGGTGGAGGGGCTGGGGGCACCAGGCGTGCTGGAGGCGGAGGGCGGGAGATTTGGGGACC
AGGCTGCACAGAACCCGTCGGAAGCAGGGCGATCAGCCGGGAGCTGCAGAGGCCTGGGGGGCCTCTAGCCCAGGGCA
GCCTGGGAGGGGCAGCTGCCTGGGCACCCGGGCCCCGCGAGGAGGGGCTGGGGCCTGCTGCGGGGTCGCAGATGTGT
CCCGGTGCTCGGAGAGGGCCGCAGGGCGCGTGGGCCGTGGCGGGAGGCCGCGCTGCTGGGAGCTCACGGCCCCCGCC
CCCCGTCCCAGGCTCTTCCTCAGCCACCCGCAGACCAAGACCTACTTCCCGCACTTCGACCTGCACCCGGGGTCCGC
GCAGTTGCGCGCGCACGGCTCCAAGGTGGTGGCCGCCGTGGGCGACGCGGTGAAGAGCATCGACGACATCGGCGGCG
CCCTGTCCAAGCTGAGCGAGCTGCACGCCTACATCCTGCGCGTGGACCCGGTCAACTTCAAGGTGCGCGGGGCGCGG
TGCGGGCGGGGCGGGGCGGGGCCGCGGGGCGGGCGGGGCCGCGGGGCGGGGTCGCGGGGCGGGGCGGGGTGGGGTCG
CGGGGCGGGGCGGGGTCGCGGGGCGGGGCGGGGCGGGGGGGGGGGGGCGGGGCGGCCGGGGCCCGGCGGGGCGGGGC
GGGGCGGGGAGGGGCTGGGCGGGGCGGGGCGCGGGGCGGGGCGGGCCGGGCCGGGGCGGGGTCGCGGGGCGGGGTCG
CGGGGCGGGGCGCGGGGCGGGGCGGGGGGGGGTGGGGTCGCGGGGCGGGGCCCGGGCTAGGCCCCGCCCCCGCACTG
AGCCGCCCCCGCCCCCAGCTCCTGTCCCACTGCCTGCTGGTCACCCTGGCCGCGCGCTTCCCCGCCGACTTCACGGC
CGAGGCCCACGCCGCCTGGGCCAAGTTCCTATCGGTCGTATCCTCTGTCCTGACCGAGAAGTACCGCTGAGCGCCGC
CTCCGGGACCCCCAGGACAGGCTGCGGCCCCTCCCCTGCCCTTCACCCTCCCACAGTTCCTGCCCTGACTCCAATAA
ATGGATGAGGACGGAGCGATCTGGGCTCTGTGTTCTCAGTATTGGAGGGAAGGAGGGGAGAAGCTGAGTGATGGGTC
CGGGGGCTTCGCAGGAACTCGGTCGTCCCCACTGTCGTCGCGGCCTGGGGTTCACTTGGGGGGCGCCTTGGGGAGGT
TCTAGCCCCTGAGCACCGGAGCTGCGGCCCGGGTGGAGCGGAGCAGTCCCGGGCCGGCCCGCGGCGTCTCCTGGGGT
CCTTGAGTCGGACGGGCGTTTGTGCGTCTCCCGGCTTCCCATATCGCACAAAGATTGTCACTTCACTAAGCGTATTG
GAAGCGTGTCGGGGCTCAGGGAACTTTTCCACAAAGCCTGACGTCCGAATCCCGGGACTCTGGCAGCTACGGGGGTC
CCTGAGGCCGGTCCCTCCCCGACTCCTAAGAGAGTAGGGGGTTTCCTGCCCGGTGTTCTCTCTCCGGTTCCTCCCAT
GTGCTCCCTCCTGGCAGAGCAGTAACTTTACCCGAGGGGAGTAAACAGATGCCCCTAAAGTCTGCAGTAAAGGTGCC
CACGCGCAACGGCGTGGGTCAATGCCAGAAACCCTGGGATCCCGGAGGTCGAGGCCTCCACACAGACGGGAACCCGG
GCTGGTTACGTTCCCCGGCGCAGGCCGAGGGTCCCCGCGTTCCCGCCGCGCTCGGGCCGATAAGGACGGGCGGGGTG
CCCGGAGGCTCTATAAGGAGGCCAGGGCGGCGGGCGCGGCCCCCAGAGCACGTCAGGCGGCGCCATGCTCAGCGCCC
AGGAGCGCGCCCAAATCGCGCAGGTCTGGGACCTGATTGCGGGCCACGAGGCGCAATTCGGGGCGGAGCTGCTGCTC
AGGTCGGTAGAGGCGGGGTCTCCGGGAGCTCAGGGAGGTGGAGATGAGGGTTTTGGGCGCGTGGGCCGCCAACGCCA
TCCAAGGTCCTTCGGGTGCGGATCCCCGGGCTCTGGGCGGTGTGGGCGCTAGTGAAGCCCCACGCAGCCGCCCTCCT
CCCCGGTCACTGACCTGGTCCTGCAGGCTCTTCACGGTGTACCCCAGCACCAAGGTCTACTTCCCGCACCTGAGCGC
CTGCCAGGACGCGACGCAGCTGCTGAGCCACGGGCAGCGCATGCTGGCGGCTGTGGGCGCGGCGGTGCAGCACGTGG
ACAACCTGCGCGCCGCGCTGAGCCCGCTGGCGGACCTGCACGCGCTCGTGCTGCGCGTGGACCCAGCCAACTTTCCG
GTGAGGCCTTTCCGGCCGGGGCAATGGTGCAGCGCGCAGCCGGGGTGGGGGGGCTCTGGGGGTCCCTAGCGGGGCAG
ACCCCGTCTCACCGGCCCCTTCTCCTGCAGCTGCTAATCCAGTGTTTCCACGTCGTGCTGGCCTCCCACCTGCAGGA
CGAGTTCACCGTGCAAATGCAAGCGGCGTGGGACAAGTTCCTGACTGGTGTGGCCGTGGTGCTGACCGAAAAATACC
GCTGAGCCCTGTGCTGCGCAGGCCTTGGTCTGTGCCTGTCAATAAACAGAGGCCCGAACCATCTGCCCCTGCCTGTG
TGGTCTTTGGGGAGCTAGCAAAGCGAGGTCACTATTGTTGGCCAGTGAAGCTCAGGGACCTAAAAGGAGCCTCCTAG
AACTCTCAAATGCGCCCCACCCCCGGAGGTTTGTCCTCCCATGGCGAGGAGTGCGATGGGGCAGAGGGAGCACTGTG
ATGTGGCGGGGGTAGGGAGGGTGGCCTTCGACTTCAACCCTTGAATCGGGCTTCCAACCATACTGTTCGCAAAGCAC
TTCCCCATTCACGCATTTATTCATTCATTCTCCCTCCATCCCCACTTCCTGCTGGGACCTGTAGATGCTAATCCTGG
CCCTTTTTGCAGAGAGATGCAGAAACTGAGGTCCCAGAGCCAAATGTGCAACCTAATTCGTTGGCCCAGAGCAGAGG
GCTCCGCAGACCTGTTCCTTTCCCCTTCCTTCCCCCATGGACACTTCCTCAGTGGCAAACCTGCGCTAGCCTGGTTA
GCCCTCCCTGTGACCCTGCAGCCCTGGGGATGAGGTCGGGAGGAAGTCCTCAGTGGCCACAATTTGGCAGACAGAGC
AGGTTTAGTCTTCCAGCCTGCTCAATGACAAGCTGTGCGACCCTGGGCGTGTCCCAGAGCTCTCAGGCCTTTACCTA
TCGAATAGAAAAACAACGTCCAACTCACGAGATTTTTGAAATAATTTTTGAAATCATAACACAGGGTGGGTGCCTGC
AGGGTCGTTGCCACCCCACCCCTCCACCCAGCCCCAGCTGCCGTGTCTCAATCTCTGCAGGTGCCCAGGCCAAGGCA
CTCCCTTCCCCAGGTTCCCTCTTCTCCCTCCCCAGGACTGGGAAGGGAATCTTAGGGCTCCACCCCAGGCTTTTCAG
ACAAAGAATAGGGGCTGAGGAAAGAGTGGGACCTTGGAGGTCTCCAAACCCTGAATAGGGTTGGCTCTGGGTTGGCC
ATCCTGGGTCTGTGTGGGGAGCACTGGACCAGGCCTGGCACCCAGGTCTGACCTGGCAGTCAGCAACGAGGTCTGAA
GAGAGCTGCTGGAAGTGGAGCCCTGACTGTGAGTCGGCCAAACTCCCCCCAGCAGTCAGTGCCAGTGACCTGTTGCC
CTGCACTGCCTGGGACCCCAGCCCGGTAGTTTGGAGAACTTGGCCCCACGTTATCTACATCCCCCAACTGTTTTTTT
GTTTTTGGGGGTTTTTTTTTTTTTTGCTTTGTTTTTGTTTTTGAGATAGGCCCTTGCTCTGACACCCCGGCTGGAGT
GCAGTGGCACAGTTTTGGCTCACTGCAGCCTCAACCTCCTGGGTTCAAGCGATTCTCCTGCCTCTGTCTCCCGTGTA
GCTGGGATTACAGGCATGGGCCGCCATTCCTGGCTAATTTTTGTATTTTTAATAGAGACACAGTTTCACCATGTTGA
TCAGGCTGGTCTCAAACTCCTGACCTCAAGTGATCTGCCCTCCTCGGTCTCCCAAAGTGCTGGGATGACAGGCGTGA
GCCACCACACCCAGCCCCCGCAACTGTTTACATGGATAATTAACAGCTTTTTGTCCCAGGCAGAGTTTGGTGTGAAA
GCAGCTTATGTTTCACTTTGGAAAAACTGTGCTCTTCTCCCCATCCAGGAAGCTGCCTGGGTCTGGGCCATATGTGG
ATACCTTATGGGTATAAGCTGCTCAGGACCCTGTGTGGAAGCTCAGGACAATGCCAGCGGGAAGGCTACCATGTGGA
GAGCTGGTCTCTGTTTGGGCAGGACTAAGAGACGCAGGGCAGCCTTGGGCAACCTGTCTACTCTCACTCACTCCTCC
TCCCCTTTCCTGTGCCAGGCACCTCCTGGCAACTTGCCAGCCAATGACCCTGCATCCCAGGCATAAGAGCTCCTACT
CTCCCCCACCTTTCACTTTTGAGCTTACACAGACTCAGAAATAAGCTGCCGTGGTGCTGTCTCCTGAGGACAAGGCT
AACACCAAGGCGGTCTGGGAGAAAGTTGGCAACCACACTGCTGGCTATGCCACGGAGGCCCTGGAGAGGCAAGAACC
CTCCTCTCCCTGCTCACACCTTGGGTCCAACGCCCACTCCAGGGCTCCACTGGCCACCCCTAACTATTCTTACCCTG
GACCCAGCCCCCAGCCCCTCACTCTTTGCTTCCCCCTGAAGCATGTTCCTGACCTTCCTCTCACTTGGCCCTGAGTT
ATGGCTCAGCCCAGATCAAGAAACAATGCAAGTAGGTGGCCGACACGCTGACCAATGCCGTGGTCCACTTAGATGAC
ATGCCCAATGATGTGTCTGAGCTGAGGAAGCTGCATGTCCACGAGCTGTGGGTGGACCCAGGCAACATCAGGGAGAG
CTTTGGGCTGGGAGGAATCTAGGGTGTGGGGGCAGCTGGCCTTCCTCATAGGACAGACCCTCCCACGCGTTCAGGGA
GGTGGAGCACAGGTGGCAGTAGTATCTGCATCCCCTGACTCTCTCTCCACAGTTCCTGGGTAAATGCCTGCTGGTGA
CCTAGGCCTGCCACACCCTTCCCAGTTTACCCATGTGGTGCCTCCATGGACAAATTATTTGCTTTTGTGAGTGCTGT
GTTGACCTAAAAACACCATTAAGCTAGAGCATTGGTGGTCATGCCCCCTGCCTGCTGGGCCTCCCACCAGGCCCTCC
TCCCCTCCCTGCCCCAGCACTTCCTGATCTTTGAATGAAGTCCGAGTAGGCAGCAGCCTGTGTGTGCCTGGGTTCTC
TCTGTCCCGGAATGTGCCAACAGTGGAGGTGTTTACCTGTCTCAGACCAAGGACCTCTCTGCAGCTGCATGGGGCTG
GGGAGGGAGAACTGCAGGGAGTATGGGAGGGGAAGCTGAGGTGGGCCTGCTCAAGAGAAGGTGCTGAACCATCCCCT
GTCCTGAGAGGTGCCAGGCCTGCAGGCAGTGGCTCAGAAGCTGGGGAGGAGAGAGGCATCCAGGGTTCTACTCAGGG
AGTCCCAGCATCGCCACCCTCCTTTGAAATCTCCCTGGTTGAACCCAGTTAACATACGCTCTCCATCAAAACAAAAC
GAAACAAAACAAACTAGCAAAATAGGCTGTCCCCAATGCAAGTGCAGGTGCCAGAACATTTCTCTCATTCTCACCCC
TTCCTGCCAGAGGGTAGGTGGCTGGAGTGAGGGTGCTGGCCCTACTCACACTTCCTGTGTCATGGTGACCCTCTGAG
AGCAGCCCAGTCAGTGGGGAAGGAGGAAGGGGCTGGGATGCTCACAGCCGGCAGCCCACACCTGGGGAGACTCTTCA
GCAGAGCACCTTGCGGCCTTACTCCTGCACGTCTCCTGCAGTTTGTAAGGTGCATTCAGAACTCACTGTGTGCCCAG
CCCTGAGCTCCCAGCTAATTGCCCCACCCAGGGCCTCTGGGACCTCCTGGTGCTTCTGCTTCCTGTGCTGCCAGCAA
CTTCTGGAAACGTCCCTGTCCCCGGTGCTGAAGTCCTGGAATCCATGCTGGGAAGTTGCACAGCCCATCTGGCTCTC
AGCCAGCCTAGGAACACGAGCAGCACTTCCAGCCCAGCCCCTGCCCCACAGCAAGCCTCCCCCTCCACACTCACAGT
ACTGAATTGAGCTTTGGGTAGGGTGGAGAGGACCCTGTCACCGCTTTTCTTCTGGACATGGACCTCTCTGAATTGTT
GGGGAGTTCCCTCCCCCTCTCCACCACCCACTCTTCCTGTGCCTCACAGCCCAGAGCATTGTTATTTCAACAGAAAC
ACTTTAAAAAATAAACTAAAATCCGACAGGCACGGTGGCTCACACCTGTAATCCCAGTACTTTGGGAGGCTGAGGCG
AGAGGATCACCTGAGGTCGGGAGTTTGAGACCAGCCTGACCAATATGGAGAAACCCCAGTTATACTAAAAATACAAA
ATTAGCTGGGTGTGGTGGCGCATGCCTGTAATCCTAGCTACTAGGAAGGCTGAGGCAGGAGAATCGCTTGAACCCGG
GAGGTGGAGGTTGAGGTGAGCTGAGATCACGCCATTGCACTCCAGCCTGGGCAACAAGAGCAAAACTCCGTCTCAAA
AAATAAATAAATAAATAAATAAATAAACTAAAATCTATCCATGCTTTCACACACACACACACACACACACACACACA
CCCTTTTTTGTGTTACTTAAAGTAGGAGAGTGTCTCTCTTTCCTGTCTCCTCACACCCACCCCCAGAAGAGACCAAA
ATGAAGGGTTTGGAACTCAGCCCATGGGCCCCATCCCATGCTGAGGGAACACAGCTACATCTACAACTACTGCCACA
GGCTCTCTTTTTGGACAAAAATACCATCATACTGTAGATACCTGTGTACAACTTCCTATTCTCAGTGAAGTGTCTCC
CCTGCATCCCTTTCAGCCAGTTCATTCAGCTCTGCGCCATTCCACAGTCTCACTGATTATTACTATGTTTCCATCAT
GATCCCCCCAAAAAATCATGACTTTATTTTTTTATTTTTATTATTATTATTTTTTTTTTTTTTTTTGTGACGGAGTC
TCGCTCTGTCACCCAGGCTGGAGTGCAGTGGCACAATCTCGGCTCACTGCAAGCTCCACCTCGCAGGTTCACGCCAT
TCTCCTCCCTCAGCCTCCCGAGTAGCTGAGTAGCTGGGACTACAGGCGCCCCCCACTACGCCTGGCTAATTTTTTCT
ATTTTTAATAGAGACAGAGTTTCACTGCATTAGCGAGGATGGTCTCGATCTCCTGACCTCGCATCTGCCCGCCTCAG
CCTCCCAATGTGCTGGGATTACAGGCGTGAGCCACCGCGCCCGGCCTTATGTATTTATTTTTTTGAGACAGAGTCTC
GCTGTGTCGTCAGGCTAGAGTGCTGTGGCACGATCTCGGCTCACTGCAACCTCCAACTCCCTGGTTCAAAGGATTCT
CCAGCCTCCACCTCCCGAGTAGCTGGGATTACAGGCGTGCACCACCACACCCAGCTAATTTTTGTATTTTTAGTAGA
GACGGGGTTTCTCCATGTTGGTCAGCCTGGTCTCGAACTCCCGACCTCAGCTGATCCACCCGCCTTGGCCTCCCAAA
GTGCTGGGATTACAGGCGTGAGCCACCGAGCCTGGCCAAACCATCACTTTTCATGAGCAGGGATGCACCCACTGGCA
CTCCTGCACCTCCCACCCTCCCCCTCGCCAAGTCCACCCCTTCCTTCCTCACCCCACATCCCCTCACCTACATTCTG
CAACCACAGGGGCCTTCTCTCCCCTGTCCTTTCCCTACCCAGAGCCAAGTTTGTTTATCTGTTTACAACCAGTATTT
ACCTAGCAAGTCTTCCATCAGATAGCATTTGGAGAGCTGGGGGTGTCACAGTGAACCACGACCTCTAGGCCAGTGGG
AGAGTCAGTCACACAAACTGTGAGTCCATGACTTGGGGCTTAGCCAGCACCCACCACCCCACGCGCCACCCCACAAC
CCCGGGTAGAGGAGTCTGAATCTGGAGCCGCCCCCAGCCCAGCCCCGTGCTTTTTGCGTCCTGGTGTTTGTTCCTTC
CCGGTGCCTGTCACTCAAGCACACTAGTGACTATCGCCAGAGGGAAAGGGAGCTGCAGGAAGCGAGGCTGGAGAGCA
GGAGGGGCTCTGCGCAGAAATTCTTTTGAGTTCCTATGGGCCAGGGCGTCCGGGTGCGCGCATTCCTCTCCGCCCCA
GGATTGGGCGAAGCCCTCCGGCTCGCACTCGCTCGCCCGTGTGTTCCCCGATCCCGCTGGAGTCGATGCGCGTCCAG
CGCGTGCCAGGCCGGGGCGGGGGTGCGGGCTGACTTTCTCCCTCGCTAGGGACGCTCCGGCGCCCGAAAGGAAAGGG
TGGCGCTGCGCTCCGGGGTGCACGAGCCGACAGCGCCCGACCCCAACGGGCCGGCCCCGCCAGCGCCGCTACCGCCC
TGCCCCCGGGCGAGCGGGATGGGCGGGAGTGGAGTGGCGGGTGGAGGGTGGAGACGTCCTGGCCCCCGCCCCGCGTG
CACCCCCAGGGGAGGCCGAGCCCGCCGCCCGGCCCCGCGCAGGCCCCGCCCGGGACTCCCCTGCGGTCCAGGCCGCG
CCCCGGGCTCCGCGCCAGCCAATGAGCGCCGCCCGGCCGGGCGTGCCCCCGCGCCCCAAGCATAAACCCTGGCGCGC
TCGCGGGCCGGCACTCTTCTGGTCCCCACAGACTCAGAGAGAACCCACCATGGTGCTGTCTCCTGCCGACAAGACCA
ACGTCAAGGCCGCCTGGGGTAAGGTCGGCGCGCACGCTGGCGAGTATGGTGCGGAGGCCCTGGAGAGGTGAGGCTCC
CTCCCCTGCTCCGACCCGGGCTCCTCGCCCGCCCGGACCCACAGGCCACCCTCAACCGTCCTGGCCCCGGACCCAAA
CCCCACCCCTCACTCTGCTTCTCCCCGCAGGATGTTCCTGTCCTTCCCCACCACCAAGACCTACTTCCCGCACTTCG
ACCTGAGCCACGGCTCTGCCCAGGTTAAGGGCCACGGCAAGAAGGTGGCCGACGCGCTGACCAACGCCGTGGCGCAC
GTGGACGACATGCCCAACGCGCTGTCCGCCCTGAGCGACCTGCACGCGCACAAGCTTCGGGTGGACCCGGTCAACTT
CAAGGTGAGCGGCGGGCCGGGAGCGATCTGGGTCGAGGGGCGAGATGGCGCCTTCCTCTCAGGGCAGAGGATCACGC
GGGTTGCGGGAGGTGTAGCGCAGGCGGCGGCTGCGGGCCTGGGCCGCACTGACCCTCTTCTCTGCACAGCTCCTAAG
CCACTGCCTGCTGGTGACCCTGGCCGCCCACCTCCCCGCCGAGTTCACCCCTGCGGTGCACGCCTCCCTGGACAAGT
TCCTGGCTTCTGTGAGCACCGTGCTGACCTCCAAATACCGTTAAGCTGGAGCCTCGGTAGCCGTTCCTCCTGCCCGC
TGGGCCTCCCAACGGGCCCTCCTCCCCTCCTTGCACCGGCCCTTCCTGGTCTTTGAATAAAGTCTGAGTGGGCAGCA
GCCTGTGTGTGCCTGGGTTCTCTCTATCCCGGAATGTGCCAACAATGGAGGTGTTTACCTGTCTCAGACCAAGGACC
TCTCTGCAGCTGCATGGGGCTGGGGAGGGAGAACTGCAGGGAGTATGGGAGGGGAAGCTGAGGTGGGCCTGCTCAAG
AGAAGGTGCTGAACCATCCCCTGTCCTGAGAGGTGCCAGGCCTGCAGGCAGTGGCTCAGAAGCTGGGGAGGAGAGAG
GCATCCAGGGTTCTACTCAGGGAGTCCCAGCATCGCCACCCTCCTTTGAAATCTCCCTGGTTGAACCCAGTTAACAT
ACGCTCTCCATCAAAACAAAACGAAACAAAACAAACTAGCAAAATAGGCTGTCCCCAGTGCAAGTGCAGGTGCCAGA
ACATTTCTCTCATTCCCACCCCTTCCTGCCAGAGGGTAGGTGGCTGGAGTGAGGGTGCTGGCCCTACTCACACTTCC
TGTGTCACGGTGACCCTCTGAGAGCAGCCCAGTCAGTGGGGAAGGAGGAAGGGGCTGGGATGCTCACAGCCGGCAGC
CCACACCTGGGGAGACTCTTCAGCAGAGCACCTTGCGGCCTTACTCCTGCACGTCTCCTGCAGTTTGTAAGGTGCAT
TCAGAACTCACTGTGTGCCCAGCCCTGAGCTCCCAGCTAATTGCCCCACCCAGGGCCTCTGGGACCTCCTGGTCTTC
TGCTTCCTGTGCTGCCAGCAACTTCTGGAAACGTCCCTGTCCCCGGTGCTGAAGTCCTGGAATCCATGCTGGGAAGT
TGCACAGCCCATCTGGCTCTCAGCCAGCCTAGGAACATGAGCAGCACTTCCAACCCAGTCCCTGCCCCACAGCAAGC
CTCCCCCTCCACACTCACAGTACTGGATTGAGCTTTGGGGAGGGTGGAGAGGACCCTGTCACTGCTTTCCTTCTGGA
CATGGACCTCTCTGAATTGTTGGGGAGTTCCCTCCCCTCTCCACCACCCGCTCTTCCTGCGCCTCACAGCCCAGAGC
ATTGTTATTTCAGCAGAAACACTTTAAAAAATAAACTAAAATCCGACAGGCACGGTGGCTCACGCCTGTAATCCCAG
CACTTTGGGAGGCCGAGGTGGGAGGATCACCTGAGGTCGGGAGTTTGAGACCACCCTGATCAACATGTAGAAACCCC
ATCTATACTAAAAATACAAAATCAGCCGGGCATGGTGGCCCATGCCTGTAAACCCACCTACTCCGGAGGCTGAGGCA
GGAGAATCATTTTAACCAAGGAGGCAGAGGTTGCAGTGAGCTAAGATCACACCATTGCACTCCAGCCTGGAAAACAA
CAGCGAAACTCCGCCTCAAAAAAAAAAAAGCCCCCACATCTTATCTTTTTTTTTTCCTTCAGGCTGTGGGCAGAGTC
AGAAGAGGGTGGCAGACAGGGAGGGGAAATGAGAAGATCCAACGGGGGAAGCATTGCTAAGCTGGTCGGAGCTACTT
CCTTCTCTGCCCAAGGCAGCTTACCCTGGCTTGCTCCTGGACACCCAGGGCAGGGCCTGAGTAAGGGCCTGGGGAGA
CAGGGCAGGGAGCAGGCTGAAGGGTGCTGACCTGATGCACTCCTCAAAGCAAGATCTTCTGCCAGACCCCCAGGAAA
TGACTTATCAGTGATTTCTCAGGCTGTTTTCTCCTCAGTACCATCCCCCCAAAAAACATCACTTTTCATGCACAGGG
ATGCACCCACTGGCACTCCTGCACCTCCCACCCTTCCCCAGAAGTCCACCCCTTCCTTCCTCACCCTGCAGGAGCTG
GCCAGCCTCATCACCCCAACATCTCCCCACCTCCATTCTCCAACCACAGGGCCCTTGTCTCCTCTGTCCTTTCCCCT
CCCCGAGCCAAGCCTCCTCCCTCCTCCACCTCCTCCACCTAATACATATCCTTAAGTCTCACCTCCTCCAGGAAGCC
CTCAGACTAACCCTGGTCACCTTGAATGCCTCGTCCACACCTCCAGACTTCCTCAGGGCCTGTGATGAGGTCTGCAC
CTCTGTGTGTACTTGTGTGATGGTTAGAGGACTGCCTACCTCCCAGAGGAGGTTGAATGCTCCAGCCGGTTCCAGCT
ATTGCTTTGTTTACCTGTTTAACCAGTATTTACCTAGCAAGTCTTCCATCAGATAGCATTTGGAGAGCTGGGGGTGT
CACAGTGAACCACGACCTCTAGGCCAGTGGGAGAGTCAGTCACACAAACTGTGAGTCCATGACTTGGGGCTTAGCCA
GCACCCACCACCCCACGCGCCACCCCACAACCCCGGGTAGAGGAGTCTGAATCTGGAGCCGCCCCCAGCCCAGCCCC
GTGCTTTTTGCGTCCTGGTGTTTATTCCTTCCCGGTGCCTGTCACTCAAGCACACTAGTGACTATCGCCAGAGGGAA
AGGGAGCTGCAGGAAGCGAGGCTGGAGAGCAGGAGGGGCTCTGCGCAGAAATTCTTTTGAGTTCCTATGGGCCAGGG
CGTCCGGGTGCGCGCATTCCTCTCCGCCCCAGGATTGGGCGAAGCCTCCCGGCTCGCACTCGCTCGCCCGTGTGTTC
CCCGATCCCGCTGGAGTCGATGCGCGTCCAGCGCGTGCCAGGCCGGGGCGGGGGTGCGGGCTGACTTTCTCCCTCGC
TAGGGACGCTCCGGCGCCCGAAAGGAAAGGGTGGCGCTGCGCTCCGGGGTGCACGAGCCGACAGCGCCCGACCCCAA
CGGGCCGGCCCCGCCAGCGCCGCTACCGCCCTGCCCCCGGGCGAGCGGGATGGGCGGGAGTGGAGTGGCGGGTGGAG
GGTGGAGACGTCCTGGCCCCCGCCCCGCGTGCACCCCCAGGGGAGGCCGAGCCCGCCGCCCGGCCCCGCGCAGGCCC
CGCCCGGGACTCCCCTGCGGTCCAGGCCGCGCCCCGGGCTCCGCGCCAGCCAATGAGCGCCGCCCGGCCGGGCGTGC
CCCCGCGCCCCAAGCATAAACCCTGGCGCGCTCGCGGCCCGGCACTCTTCTGGTCCCCACAGACTCAGAGAGAACCC
ACCATGGTGCTGTCTCCTGCCGACAAGACCAACGTCAAGGCCGCCTGGGGTAAGGTCGGCGCGCACGCTGGCGAGTA
TGGTGCGGAGGCCCTGGAGAGGTGAGGCTCCCTCCCCTGCTCCGACCCGGGCTCCTCGCCCGCCCGGACCCACAGGC
CACCCTCAACCGTCCTGGCCCCGGACCCAAACCCCACCCCTCACTCTGCTTCTCCCCGCAGGATGTTCCTGTCCTTC
CCCACCACCAAGACCTACTTCCCGCACTTCGACCTGAGCCACGGCTCTGCCCAGGTTAAGGGCCACGGCAAGAAGGT
GGCCGACGCGCTGACCAACGCCGTGGCGCACGTGGACGACATGCCCAACGCGCTGTCCGCCCTGAGCGACCTGCACG
CGCACAAGCTTCGGGTGGACCCGGTCAACTTCAAGGTGAGCGGCGGGCCGGGAGCGATCTGGGTCGAGGGGCGAGAT
GGCGCCTTCCTCGCAGGGCAGAGGATCACGCGGGTTGCGGGAGGTGTAGCGCAGGCGGCGGCTGCGGGCCTGGGCCC
TCGGCCCCACTGACCCTCTTCTCTGCACAGCTCCTAAGCCACTGCCTGCTGGTGACCCTGGCCGCCCACCTCCCCGC
CGAGTTCACCCCTGCGGTGCACGCCTCCCTGGACAAGTTCCTGGCTTCTGTGAGCACCGTGCTGACCTCCAAATACC
GTTAAGCTGGAGCCTCGGTGGCCATGCTTCTTGCCCCTTGGGCCTCCCCCCAGCCCCTCCTCCCCTTCCTGCACCCG
TACCCCCGTGGTCTTTGAATAAAGTCTGAGTGGGCGGCAGCCTGTGTGTGCCTGAGTTTTTTCCCTCAGCAAACGTG
CCAGGCATGGGCGTGGACAGCAGCTGGGACACACATGGCTAGAACCTCTCTGCAGCTGGATAGGGTAGGAAAAGGCA
GGGGCGGGAGGAGGGGATGGAGGAGGGAAAGTGGAGCCACCGCGAAGTCCAGCTGGAAAAACGCTGGACCCTAGAGT
GCTTTGAGGATGCATTTGCTCTTTCCCGAGTTTTATTCCCAGACTTTTCAGATTCAATGCAGGTTTGCTGAAATAAT
GAATTTATCCATCTTTACGTTTCTGGGCACTCTTGTGCCAAGAACTGGCTGGCTTTCTGCCTGGGACGTCACTGGTT
TCCCAGAGGTCCTCCCACATATGGGTGGTGGGTAGGTCAGAGAAGTCCCACTCCAGCATGGCTGCATTGATCCCCCA
TCGTTCCCACTAGTCTCCGTAAAACCTCCCAGATACAGGCACAGTCTAGATGAAATCAGGGGTGCGGGGTGCAACTG
CAGGCCCCAGGCAATTCAATAGGGGCTCTACTTTCACCCCCAGGTCACCCCAGAATGCTCACACACCAGACACTGAC
GCCCTGGGGCTGTCAAGATCAGGCGTTTGTCTCTGGGCCCAGCTCAGGGCCCAGCTCAGCACCCACTCAGCTCCCCT
GAGGCTGGGGAGCCTGTCCCATTGCGACTGGAGAGGAGAGCGGGGCCACAGAGGCCTGGCTAGAAGGTCCCTTCTCC
CTGGTGTGTGTTTTCTCTCTGCTGAGCAGGCTTGCAGTGCCTGGGGTATCAGAGGGAGGGTTCCCGGAGCTGGTAGC
CATAAAGCCCTGGCCCTCAACTGATAGGAATATCTTTTATTCCCTGAGCCCATGAATCACCCTTGGTAAACACCTAT
GGCAGGCCCTCTGCCTGCGTTTGTGATGTCCTTCCCGCAGCCTGTGGGTACAGTATCAACTGTCAGGAAGACGGTGT
CTTCGTTATTTCATCAGGAAGAATGGAGGTCTGACCTAAAGGTAGAAATATGTCAAATGTACAGCAGAGGGCTGGTT
GGAGTGCAGCGCTTTTTACAATTAATTGATCAGAACCAGTTATAAATTTATCATTTCCTTCTCCACTCCTGCTGCTT
CAGTTGACTAAGCCTAAGAAAAAATTATAAAAATTGGCCGGGCGCGGTGGCTCACACCTGTAATTGCAGCACTTTGC
CAGGCTTAGGCAGGTGGATCACCTGAAGTCAGGGGTTCGAGACCAGCCTAGCCAACATAGTGAAACCCTGTCTCTAC
TAAAAAGACAAAAATTGTCCAGGTGTGATGACTCATGCCTGTAAACCTGGCACTTTGGGAGGCGGAGGTTGTAGTGA
GTCAAGATCGCGCCATCGCACTCCAGCTTGGGCAACAAGAGCGAAACTCTGTCTCAAAAAAAAATTTAATCTAATTT
AATTTAATTTAAAAATTAGCACGGTGGTTGGGCACAGTGGCTCACGCCTGTAATCCCAGCACTTTGGGAAGCCAAGG
TGGGCAGATCACAAGGTCAGGAATTCGAGACCAGCCTGGCCAATATGGGGAAACCCCATCTCTACTAAAAATACAAA
AAATTAGCCGGGTGTGGTGGCGCACGCCTGTAATCCCAGCTACTCGGGAGGTTGAGGTAGGAGAATCACTTGAACCC
AGGAGGCAGAGGTTGCAGTGACCCGAGATCACACCATTGCACTCTAGCCTGGGCAACAAGAGCAAAACTCCATCTCA
AAAAAAATTATAAAAATTATACATCAGTAGATGAATGGGTAAACAAAATGTGGTGGTCTATACACACAATGGAATAT
TATTTGGCCACAAAAAGAAATGAAGCACTGATAGGATGTAGCTGCACCCTGAAAATATTTGACAAGTAAAAGAAGCC
GGACACCAAAGGTCACAAACTGCATGACCCCATCTATATGCAATATCCGCTACAGCCAAATCCATAGGGACCAAAAG
CGGATTAGTGGCTGCCGGGGCCAGAGTTACTGTTAATGAGTACCGAGGTGGCGTTTGGGATGATGAAAAAGTTCTGA
CCTAGATAGTGGTGATGGCTGCATAACACTAAGTGTTCTTAATATCACCAAATTTTATACCTGAAAAATGGCTACAA
TGGTAATTTATGTCTATTTTATCACCTTTTTTAAAACAAAAAAGATATAAGGGGTACAGCAGAGTGAGTGCTGCATA
TGCATTTACTATTATTCTTGGGTTACATCCCAGGTACTCAATAAATGTTCACTGCCCTGAAGAAACACCTGCTACGA
GTCAGGCACCTCACAGTTGTTATCCGTTTAATTCTCACAATCTGAGAAGAAACTGTCACCCTCATTTTATATAATAA
ATGAGAAAACAGACTCGGGCAAGTGTCACAATAGAATCAAGAGGCAGAATAAACTGACTTCCAATGCCAAATCCATG
CCGAAATTCAGTGCTATAATAATGTACATGGCCGGGCGCGGTGGTTCACGCCTGTAATCCCAGAACTTTGGGAGGCT
GAGGCGGGAGGATCACCTGAGGTCGGGAGTTTGAGATCAGCCTAACACGGTGAAACCCTGTCTCTACTAAAAATACA
AAATTGGCATGGTGGCATGCACCTGTGATCCCAGTTACTCGGGAGGCTGAGGCAGGAGAATCGTTTGAACCCGGGAG
GCGGAGGTTGCAGTGAGCCGGAATGGCGCCACTGCACTCACCGCACCCGGCCAATTTTTGTGTTTTTAGTAGAGACT
AAATACCATATAGTGAACACCTAAGACGGGGGGCCTTGGATCCAGGGCGATTCAGAGGGCCCCGGTCGGAGCTGTCG
GAGATTGAGCGCGCGCGGTCCCGGGATCTCCGACGAGGCCCTGGACCCCCGGGCGGCGAAGCTGCGGCGCGGCGCCC
CCTGGAGGCCGCGGGACCCCTGGCCGGTCCGCGCAGGCGCAGCGGGGTCGCAGGGCGCGGCGGGTTCCAGCGCGGGG
ATGGCGCTGTCCGCGGAGGACCGGGCGCTGGTGCGCGCCCTGTGGAAGAAGCTGGGCAGCAACGTCGGCGTCTACAC
GACAGAGGCCCTGGAAAGGTGCGGCAGGCTGGGCGCCCCCGCCCCCAGGGGCCCTCCCTCCCCAAGCCCCCCGGACG
CGCCTCACCCACGTTCCTCTCGCAGGACCTTCCTGGCTTTCCCCGCCACGAAGACCTACTTCTCCCACCTGGACCTG
AGCCCCGGCTCCTCACAAGTCAGAGCCCACGGCCAGAAGGTGGCGGACGCGCTGAGCCTCGCCGTGGAGCGCCTGGA
CGACCTACCCCACGCGCTGTCCGCGCTGAGCCACCTGCACGCGTGCCAGCTGCGAGTGGACCCGGCCAGCTTCCAGG
TGAGCGGCTGCCGTGCTGGGCCCCTGTCCCCGGGAGGGCCCCGGCGGGGTGGGTGCGGGGGGCGTGCGGGGCGGGTG
CAGGCGAGTGAGCCTTGAGCGCTCGCCGCAGCTCCTGGGCCACTGCCTGCTGGTAACCCTCGCCCGGCACTACCCCG
GAGACTTCAGCCCCGCGCTGCAGGCGTCGCTGGACAAGTTCCTGAGCCACGTTATCTCGGCGCTGGTTTCCGAGTAC
CGCTGAACTGTGGGTGGGTGGCCGCGGGATCCCCAGGCGACCTTCCCCGTGTTTGAGTAAAGCCTCTCCCAGGAGCA
GCCTTCTTGCCGTGCTCTCTCGAGGTCAGGACGCGAGAGGAAGGCGCCGCCCCTCCCCAAGGAAAGGCGAGGGCCTG
GGGCACACCCCCAGTGCCCAGATCCAGGCGCGCCTCTTTCCACCTCCAGCAGGTTTGGGGCCTCGGCCATGGGGGCA
CCGAACTGCGTGCAGCCTGACCCTCCCGAATGGGGTGGTAGGTGAGGGCCGCGGGACGCCCCGGGCGGCGGGCTGCG
AGGACGGCCGACTCTGCCCATCCCGAGGGCGGCTGGCTTCGCCCTCCCCACTCTGCGCCGAGCACGCGGCCCGGACC
CACCGCGAGAACTCCGCACCTGCAGCGTGAACGCACGCGGGCGGCGTTAAGGGCCCGGGGCTGACTCGGAGCAGGTT
AGGGAACAGCGCCCCCTCCCGGCGCGAGCCGGTACCTGCGCAGCACCCAGCCGCCGCGGCTGTGGCCTGGAATCGGG
GACCTGGGGTGCCGGGGGGTTGTGGTGAAGGAGGTGGGACCAGCCCCAGCACCTAGCCACGTAGCTGGCGAGGTGGA
CCAGGAACCGACCCAGACCCCTGCCGTCACCCGACATCACTACGGAGAGTGAAGCTTTTTTATATTTGTCCACATAA
AACCAATCATGGTCATTGTAGAACTTCCGAAAACAAGGCTTGCTGCACCTTCCTGTGTATCCCAGGTCCAGGAATGG
GTGCAGCACATCCTTCAGCTGCCGCTTGACACGCGGCAAACTGTGTCATGTGTAAACAAGAACAGGACATGGCTGTC
ATATCCAAGAGCACATGTGTAACACAGACATGCCACACACACACACACACACACACGGGGTAGAGGCAGGCCTCATC
CACACCCCTAACATTTGATGCGTAGCTGTTCCAGTCTTCTAGGCACATGTAGAGATGCTTTTCCTCAGAAATGGTAT
TCTCAAGGTGACACTGAGGAAAAGTGGACAGGCCGGGCGCGGTGGCTCACGCCTGTAATCCCAGCACTCCGGGAGGC
CGAGGCGGGCGGATC

GenBank Accession No. NM_000517 (SEQ ID NO: 247)

ACTCTTCTGGTCCCCACAGACTCAGAGAGAACCCACCATGGTGCTGTCT
CCTGCCGACAAGACCAACGTCAAGGCCGCCTGGGGTAAGGTCGGCGCGC
ACGCTGGCGAGTATGGTGCGGAGGCCCTGGAGAGGATGTTCCTGTCCTT
CCCCACCACCAAGACCTACTTCCCGCACTTCGACCTGAGCCACGGCTCT
GCCCAGGTTAAGGGCCACGGCAAGAAGGTGGCCGACGCGCTGACCAACG
CCGTGGCGCACGTGGACGACATGCCCAACGCGCTGTCCGCCCTGAGCGA
CCTGCACGCGCACAAGCTTCGGGTGGACCCGGTCAACTTCAAGCTCCTA
AGCCACTGCCTGCTGGTGACCCTGGCCGCCCACCTCCCCGCCGAGTTCA
CCCCTGCGGTGCACGCCTCCCTGGACAAGTTCCTGGCTTCTGTGAGCAC
CGTGCTGACCTCCAAATACCGTTAAGCTGGAGCCTCGGTAGCCGTTCCT
CCTGCCCGCTGGGCCTCCCAACGGGCCCTCCTCCCCTCCTTGCACCGGC
CCTTCCTGGTCTTTGAATAAAGTCTGAGTGGGCAGCA

GenBank Accession No. J00179 (SEQ ID NO: 248)

GAATTCTAATCTCCCTCTCAACCCTACAGTCACCCATTTGGTATATTAAAGATGTGTTGTCTACTGTCTAGTATCCC
TCAAGTAGTGTCAGGAATTAGTCATTTAAATAGTCTGCAAGCCAGGAGTGGTGGCTCATGTCTGTAATTCCAGCACT
GGAGAGGTAGAAGTGGGAGGACTGCTTGAGCTCAAGAGTTTGATATTATCCTGGACAACATAGCAAGACCTCGTCTC
TACTTAAAAAAAAAAAAATTAGCCAGGCATGTGATGTACACCTGTAGTCCCAGCTACTCAGGAGGCCGAAATGGGAG
GATCCCTTGAGCTCAGGAGGTCAAGGCTGCAGTGAGACATGATCTTGCCACTGCACTCCAGCCTGGACAGCAGAGTG
AAACCTTGCCTCACGAAACAGAATACAAAAACAAACAAACAAAAAACTGCTCCGCAATGCGCTTCCTTGATGCTCTA
CCACATAGGTCTGGGTACTTTGTACACATTATCTCATTGCTGTTCGTAATTGTTAGATTAATTTTGTAATATTGATA
TTATTCCTAGAAAGCTGAGGCCTCAAGATGATAACTTTTATTTTCTGGACTTGTAATAGCTTTCTCTTGTATTCACC
ATGTTGTAACTTTCTTAGAGTAGTAACAATATAAAGTTATTGTGAGTTTTTGCAAACACAGCAAACACAACGACCCA
TATAGACATTGATGTGAAATTGTCTATTGTCAATTTATGGGAAAACAAGTATGTACTTTTTCTACTAAGCCATTGAA
ACAGGAATAACAGAACAAGATTGAAAGAATACATTTTCCGAAATTACTTGAGTATTATACAAAGACAAGCACGTGGA
CCTGGGAGGAGGGTTATTGTCCATGACTGGTGTGTGGAGACAAATGCAGGTTTATAATAGATGGGATGGCATCTAGC
GCAATGACTTTGCCATCACTTTTAGAGAGCTCTTGGGGACCCCAGTACACAAGAGGGGACGCAGGGTATATGTAGAC
ATCTCATTCTTTTTCTTAGTGTGAGAATAAGAATAGCCATGACCTGAGTTTATAGACAATGAGCCCTTTTCTCTCTC
CCACTCAGCAGCTATGAGATGGCTTGCCCTGCCTCTCTACTAGGCTGACTCACTCCAAGGCCCAGCAATGGGCAGGG
CTCTGTCAGGGCTTTGATAGCACTATCTGCAGAGCCAGGGCCGAGAAGGGGTGGACTCCAGAGACTCTCCCTCCCAT
TCCCGAGCAGGGTTTGCTTATTTATGCATTTAAATGATATATTTATTTTAAAAGAAATAACAGGAGACTGCCCAGCC
CTGGCTGTGACATGGAAACTATGTAGAATATTTTGGGTTCCATTTTTTTTTCCTTCTTTCAGTTAGAGGAAAAGGGG
CTCACTGCACATACACTAGACAGAAAGTCAGGAGCTTTGAATCCAAGCCTGATCATTTCCATGTCATACTGAGAAAG
TCCCCACCCTTCTCTGAGCCTCAGTTTCTCTTTTTATAAGTAGGAGTCTGGAGTAAATGATTTCCAATGGCTCTCAT
TTCAATACAAAATTTCCGTTTATTAAATGCATGAGCTTCTGTTACTCCAAGACTGAGAAGGAAATTGAACCTGAGAC
TCATTGACTGGCAAGATGTCCCCAGAGGCTCTCATTCAGCAATAAAATTCTCACCTTCACCCAGGCCCACTGAGTGT
CAGATTTGCATGCACTAGTTCACGTGTGTAAAAAGGAGGATGCTTCTTTCCTTTGTATTCTCACATACCTTTAGGAA
AGAACTTAGCACCCTTCCCACACAGCCATCCCAATAACTCATTTCAGTGACTCAACCCTTGACTTTATAAAAGTCTT
GGGCAGTATAGAGCAGAGATTAAGAGTACAGATGCTGGAGCCAGACCACCTGAGTGATTAGTGACTCAGTTTCTCTT
AGTAATTGTATGACTCAGTTTCTTCATCTGTAAAATGGAGGGTTTTTTAATTAGTTTGTTTTTGAGAAAGGGTCTCA
CTCTGTCACCCAAATGGGAGTGTAGTGGCAAAATCTCGGCTCACTGCAACTTGCACTTCCCAGGCTCAAGCGGTCCT
CCCACCTCAACATCCTGAGTAGCTGGAACCACAGGTACACACCACCATACCTCGCTAATTTTTTGTATTTTTGGTAG
AGATGGGGTTTCACATGTTACACAGGATGGTCTCAGACTCCGGAGCTCAAGCAATCTGCCCACCTCAGCCTTCCAAA
GTGCTGGGATTATAAGCATGATTACAGGAGTTTTAACAGGCTCATAAGATTGTTCTGCAGCCCGAGTGAGTTAATAC
ATGCAAAGAGTTTAAAGCAGTGACTTATAAATGCTAACTACTCTAGAAATGTTTGCTAGTATTTTTTGTTTAACTGC
AATCATTCTTGCTGCAGGTGAAAACTAGTGTTCTGTACTTTATGCCCATTCATCTTTAACTGTAATAATAAAAATAA
CTGACATTTATTGAAGGCTATCAGAGACTGTAATTAGTGCTTTGCATAATTAATCATATTTAATACTCTTGGATTCT
TTCAGGTAGATACTATTATTATCCCCATTTTACTACAGTTAAAAAAACTACCTCTCAACTTGCTCAAGCATACACTC
TCACACACACAAACATAAACTACTAGCAAATAGTAGAATTGAGATTTGGTCCTAATTATGTCTTTGCTCACTATCCA
ATAAATATTTATTGACATGTACTTCTTGGCAGTCTGTATGCTGGATGCTGGGGATACAAAGATGTTTAAATTTAAGC
TCCAGTCTCTGCTTCCAAAGGCCTCCCAGGCCAAGTTATCCATTCAGAAAGCATTTTTTACTCTTTGCATTCCACTG
TTTTTCCTAAGTGACTAAAAAATTACACTTTATTCGTCTGTGTCCTGCTCTGGGATGATAGTCTGACTTTCCTAACC
TGAGCCTAACATCCCTGACATCAGGAAAGACTACACCATGTGGAGAAGGGGTGGTGGTTTTGATTGCTGCTGTCTTC
AGTTAGATGGTTAACTTTGTGAAGTTGAAAACTGTGGCTCTCTGGTTGACTGTTAGAGTTCTGGCACTTGTCACTAT
GCCTATTATTTAACAAATGCATGAATGCTTCAGAATATGGGAATATTATCTTCTGGAATAGGGAATCAAGTTATATT
ATGTAACCCAGGATTAGAAGATTCTTCTGTGTGTAAGAATTTCATAAACATTAAGCTGTCTAGCAAAAGCAAGGGCT
TGGAAAATCTGTGAGCTCCTCACCATATAGAAAGCTTTTAACCCATCATTGAATAAATCCCTATAGGGGATTICTAC
CCTGAGCAAAAGGCTGGTCTTGATTAATTCCCAAACTCATATAGCTCTGAGAAAGTCTATGCTGTTAACGTTTTCTT
GTCTGCTACCCCATCATATGCACAACAATAAATGCAGGCCTAGGCATGACTGAAGGCTCTCTCATAATTCTTGGTTG
CATGAATCAGATTATCAACAGAAATGTTGAGACAAACTATGGGGAAGCAGGGTATGAAAGAGCTCTGAATGAAATGG
AAACCGCAATGCTTCCTGCCCATTCAGGGCTCCAGCATGTAGAAATCTGGGGCTTTGTGAAGACTGGCTTAAAATCA
GAAGCCCCATTGGATAAGAGTAGGGAAGAACCTAGAGCCTACGCTGAGCAGGTTTCCTTCATGTGACAGGGAGCCTC
CTGCCCCGAACTTCCAGGGATCCTCTCTTAAGTGTTTCCTGCTGGAATCTCCTCACTTCTATCTGGAAATGGTTTCT
CCACAGTCCAGCCCCTGGCTAGTTGAAAGAGTTACCCATGCAGAGGCCCTCCTAGCATCCAGAGACTAGTGCTTAGA
TTCCTACTTTCAGCGTTGGACAACCTGGATCCACTTGCCCAGTGTTCTTCCTTAGTTCCTACCTTCGACCTTGATCC
TCCTTTATCTTCCTGAACCCTGCTGAGATGATCTATGTGGGGAGAATGGCTTCTTTGAGAAACATCTTCTTCGTTAG
TGGCCTGCCCCTCATTCCCACTTTAATATCCAGAATCACTATAAGAAGAATATAATAAGAGGAATAACTCTTATTAT
AGGTAAGGGAAAATTAAGAGGCATACGTGATGGGATGAGTAAGAGAGGAGAGGGAAGGATTAATGGATGATAAAATC
TACTACTATTTGTTGAGACCTTTTATAGTCTAATCAATTTTGCTATTGTTTTCCATCCTCACGCTAACTCCATAAAA
AAACACTATTATTATCTTTATTTTGCCATGACAAGACTGAGCTCAGAAGAGTCAAGCATTTGCCTAAGGTCGGACAT
GTCAGAGGCAGTGCCAGACCTATGTGAGACTCTGCAGCTACTGCTCATGGGCCCTGTGCTGCACTGATGAGGAGGAT
CAGATGGATGGGGCAATGAAGCAAAGGAATCATTCTGTGGATAAAGGAGACAGCCATGAAGAAGTCTATGACTGTAA
ATTTGGGAGCAGGAGTCTCTAAGGACTTGGATTTCAAGGAATTTTGACTCAGCAAACACAAGACCCTCACGGTGACT
TTGCGAGCTGGTGTGCCAGATGTGTCTATCAGAGGTTCCAGGGAGGGTGGGGTGGGGTCAGGGCTGGCCACCAGCTA
TCAGGGCCCAGATGGGTTATAGGCTGGCAGGCTCAGATAGGTGGTTAGGTCAGGTTGGTGGTGCTGGGTGGAGTCCA
TGACTCCCAGGAGCCAGGAGAGATAGACCATGAGTAGAGGGCAGACATGGGAAAGGTGGGGGAGGCACAGCATAGCA
GCATTTTTCATTCTACTACTACATGGGACTGCTCCCCTATACCCCCAGCTAGGGGCAAGTGCCTTGACTCCTATGTT
TTCAGGATCATCATCTATAAAGTAAGAGTAATAATTGTGTCTATCTCATAGGGTTATTATGAGGATCAAAGGAGATG
CACACTCTCTGGACCAGTGGCCTAACAGTTCAGGACAGAGCTATGGGCTTCCTATGTATGGGTCAGTGGTCTCAATG
TAGCAGGCAAGTTCCAGAAGATAGCATCAACCACTGTTAGAGATATACTGCCAGTCTCAGAGCCTGATGTTAATTTA
GCAATGGGCTGGGACCCTCCTCCAGTAGAACCTTCTAACCAGCTGCTGCAGTCAAAGTCGAATGCAGCTGGTTAGAC
TTTTTTTAATGAAAGCTTAGCTTTCATTAAAGATTAAGCTCCTAAGCAGGGCACAGATGAAATTGTCTAACAGCAAC
TTTGCCATCTAAAAAAATCTGACTTCACTGGAAACATGGAAGCCCAAGGTTCTGAACATGAGAAATTTTTAGGAATC
TGCACAGGAGTTGAGAGGGAAACAAGATGGTGAAGGGACTAGAAACCACATGAGAGACACGAGGAAATAGTGTAGAT
TTAGGCTGGAGGTAAATGAAAGAGAAGTGGGAATTAATACTTACTGAAATCTTTCTATATGTCAGGTGCCATTTTAT
GATATTTAATAATCTCATTACATATGGTAATTCTGTGAGATATGTATTATTGAACATACTATAATTAATACTAATGA
TAAGTAACACCTCTTGAGTACTTAGTATATGCTAGAATCAAATTTAAGTTTATCATATGAGGCCGGGCACGGTGGCT
CATATATGGGATTACATGCCTGTAATCCCAGCACTTTGGGAGGCCAAGGCAATTGGATCACCTGAGGTCAGGAGTTC
CAGACCAGCCTGGCCAACATGGTGAAACCCCTTCTCTACTAAAAAATACAAAAAATCAGCCAGGTGTGGTGGCACGC
GTCTATAATCCCAGCTACTCAGGAGGCTGAGGCAGGAGAATCACTTGAACCCAGGAGGTGGAGGTTGCAGTGAGCTA
AGATTGCACCACTGCACTCCAGCCTAGGCGACAGAGTGAGACTCCATCTCAAAAAAAAAAAAAGAAGTTTATTATAT
GAATTAACTTAGTTTTACTCACACCAATACTCAGAAGTAGATTATTACCTCATTTATTGATGAGGAGCCCAATGTAC
TTGTAGTGTAGATCAACTTATTGAAAGCACAAGCTAATAAGTAGACAATTAGTAATTAGAAGTCAGATGGTCTGAGC
TCTCCTACTGTCTACATTACATGAGCTCTTATTAACTGGGGACTCGAAAATCAAAGACATGAAATAATTTGTCCAAG
CTTACAGAACCACCAAGTAGTAAGGCTAGGATGTAGACCCAGTTCTGCTACCTCTGAAGACAGTGTTTTTTCCACAG
CAAAACACAAACTCAGATATTGTGGATGCGAGAAATTAGAAGTAGATATTCCTGCCCTGTGGCCCTTGCTTCTTACT
TTTACTTCTTGGCGATTGGAAGTIGTGGTCCAAGCCACAGTTGCAGACCATACTTCCTCAACCATAATTGCATTTCT
TCAGGAAAGTTTGAGGGAGAAAAAGGTAAAGAAAAATTTAGAAACAACTTCAGAATAAAGAGATTTTCTCTTGGGTT
ACAGAGATTGTCATATGACAAATTATAAGCAGACACTTGAGAAAACTGAAGGCCCATGCCTGCCCAAATTACCCTTT
GACCCCTTGGTCAAGCTGCAACTTTGGTTAAAGGGAGTGTTTATGTGTTATAGTGTTCATTTACTCTTCTGGTCTAA
CCCATTGGCTCCGTCTTCATCCTGCAGTGACCTCAGTGCCTCAGAAACATACATATGTTTGTCTAGTTTAAGTTTGT
GTGAAATTCTAACTAGCGTCAAGAACTGAGGGCCCTAAACTATGCTAGGAATAGTGCTGTGGTGCTGTGATAGGTAC
ACAAGAAATGAGAAGAAACTGCAGATTCTCTGCATCTCCCTTTGCCGGGTCTGACAACAAAGTTTCCCCAAATTTTA
CCAATGCAAGCCATTTCTCCATATGCTAACTACTTTAAAATCATTTGGGGCTTCACATTGTCTTTCTCATCTGTAAA
AAGAATGGAAGAACTCATTCCTACAGAACTCCCTATGTCTTCCCTGATGGGCTAGAGTTCCTCTTTCTCAAAAATTA
GCCATTATTGTATTTCCTTCTAAGCCAAAGCTCAGAGGTCTTGTATTGCCCAGTGACATGCACACTGGTCAAAAGTA
GGCTAAGTAGAAGGGTACTTTCACAGGAACAGAGAGCAAAAGAGGTGGGTGAATGAGAGGGTAAGTGAGAAAAGACA
AATGAGAAGTTACAACATGATGGCTTGTTGTCTAAATATCTCCTAGGGAATTATTGTGAGAGGTCTGAATAGTGTTG
TAAAATAAGCTGAATCTGCTGCCTAACATTAACAGTCAAGAAATACCTCCGAATAACTGTACCTCCAATTATTCTTT
AAGGTAGCATGCAACTGTAATAGTTGCATGTATATATTTATCATAATACTGTAACAGAAAACACTTACTGAATATAT
ACTGTGTCCCTAGTTCTTTACACAATAAACTAATCTCATCCTCATAATTCTATTAGCTAATACATATTATCATCCTA
TATTTCAGAGACTTCAAGAAGTTAAGCAACTTGCTCAAGATCATCTAAGAAGTAGGTGGTATTTCTGGGCTCATTTG
GCCCCTCCTAATCTCTCATGGCAACATGGCTGCCTAAAGTGTTGATTGCCTTAATTCATCAGGGATGGGCTCATACT
CACTGCAGACCTTAACTGGCATCCTCTTTTCTTATGTGATCTGCCTGACCCTAGTAGAACTTATGAAATTTCTGATG
AGAAAGGAGAGAGGAGAAAGGCAGAGCTGACTGTGATGAGTGATGAAGGTGCCTTCTCATCTGGGTACCAGTGGGGC
CTCTAAGACTAAGTCACTCTGTCTCACTGTGTCTTAGCCAGTTCCTTACAGCTTGCCCTGATGGGAGATAGAGAATG
GGTATCCTCCAACAAAAAAATAAATTTTCATTTCTCAAGGTCCAACTTATGTTTTCTTAATTTTTAAAAAAATCTTG
ACCATTCTCCACTCTCTAAAATAATCCACAGTGAGAGAAACATTCTTTTCCCCCATCCCATAAATACCTCTATTAAA
TATGGAAAATCTGGGCATGGTGTCTCACACCTGTAATCCCAGCACTTTGGGAGGCTGAGGTGGGTGGACTGCTTGGA
GCTCAGGAGTTCAAGACCATCTTGGACAACATGGTGATACCCTGCCTCTACAAAAAGTACAAAAATTAGCCTGGCAT
GGTGGTGTGCACCTGTAATCCCAGCTATTAGGGTGGCTGAGGCAGGAGAATTGCTTGAACCCGGGAGGCGGAGGTTG
CAGTGAGCTGAGATCGTGCCACTGCACTCCAGCCTGGGGGACAGAGCACATTATAATTAACTGTTATTTTTTACTTG
GACTCTTGTGGGGAATAAGATACATGTTTTATTCTTATTTATGATTCAAGCACTGAAAATAGTGTTTAGCATCCAGC
AGGTGCTTCAAAACCATTTGCTGAATGATTACTATACTTTTTACAAGCTCAGCTCCCTCTATCCCTTCCAGCATCCT
CATCTCTGATTAAATAAGCTTCAGTTTTTCCTTAGTTCCTGTTACATTTCTGTGTGTCTCCATTAGTGACCTCCCAT
AGTCCAAGCATGAGCAGTTCTGGCCAGGCCCCTGTCGGGGTCAGTGCCCCACCCCCGCCTTCTGGTTCTGTGTAACC
TTCTAAGCAAACCTTCTGGCTCAAGCACAGCAATGCTGAGTCATGATGAGTCATGCTGAGGCTTAGGGTGTGTGCCC
AGATGTTCTCAGCCTAGAGTGATGACTCCTATCTGGGTCCCCAGCAGGATGCTTACAGGGCAGATGGCAAAAAAAAG
GAGAAGCTGACCACCTGACTAAAACTCCACCTCAAACGGCATCATAAAGAAAATGGATGCCTGAGACAGAATGTGAC
ATATTCTAGAATATATTATTTCCTGAATATATATATATATATATATACACATATACGTATATATATATATATATATA
TATTTGTTGTTATCAATTGCCATAGAATGATTAGTTATTGTGAATCAAATATTTATCTTGCAGGTGGCCTCTATACC
TAGAAGCGGCAGAATCAGGCTTTATTAATACATGTGTATAGATTTTTAGGATCTATACACATGTATTAATATGAAAC
AAGGATATGGAAGAGGAAGGCATGAAAACAGGAAAAGAAAACAAACCTTGTTTGCCATTTTAAGGCACCCCTGGACA
GCTAGGTGGCAAAAGGCCTGTGCTGTTAGAGGACACATGCTCACATACGGGGTCAGATCTGACTTGGGGTGCTACTG
GGAAGCTCTCATCTTAAGGATACATCTCAGGCCAGTCTTGGTGCATTAGGAAGATGTAGGCAACTCTGATCCTGAGA
GGAAAGAAACATTCCTCCAGGAGAGCTAAAAGGGTTCACCTGTGTGGGTAACTGTGAAGGACTACAAGAGGATGAAA
AACAATGACAGACAGACATAATGCTTGTGGGAGAAAAAACAGGAGGTCAAGGGGATAGAGAAGGCTTCCAGAAGAAT
GGCTTTGAAGCTGGCTTCTGTAGGAGTTCACAGTGGCAAAGATGTTTCAGAAATGTGACATGACTTAAGGAACTATA
CAAAAAGGAACAAATTTAAGGAGAGGCAGATAAATTAGTTCAACAGACATGCAAGGAATTTTCAGATGAATGTTATG
TCTCCACTGAGCTTCTTGAGGTTAGCAGCTGTGAGGGTTTTGCAGGCCCAGGACCCATTACAGGACCTCACGTATAC
TTGACACTGTTTTTTGTATTCATTTGTGAATGAATGACCTCTTGTCAGTCTACTCGGTTTCGCTGTGAATGAATGAT
GTCTTGTCAGCCTACTTGGTTTCGCTAAGAGCACAGAGAGAAGATTTAGTGATGCTATGTAAAAACTTCCTTTTTGG
TTCAAGTGTATGTTTGTGATAGAAATGAAGACAGGCTACATGATGCATATCTAACATAAACACAAACATTAAGAAAG
GAAATCAACCTGAAGAGTATTTATACAGATAACAAAATACAGAGAGTGAGTTAAATGTGTAATAACTGTGGCACAGG
CTGGAATATGAGCCATTTAAATCACAAATTAATTAGAAAAAAAACAGTGGGGAAAAAATTCCATGGATGGGTCTAGA
AAGACTAGCATTGTTTTAGGTTGAGTGGCAGTGTTTAAAGGGTGATATCAGACTAAACTTGAAATATGTGGCTAAAT
AACTAGAATACTCTTTATTTTTTCGTATCATGAATAGCAGATATAGCTTGATGGCCCCATGCTTGGTTTAACATCCT
TGCTGTTCCTGACATGAAATCCTTAATTTTTGACAAAGGGGCTATTCATTTTCATTTTATATTGGGCCTAGAAATTA
TGTAGATGGTCCTGAGGAAAAGTTTATAGCTTGTCTATTTCTCTCTCTAACATAGTTGTCAGCACAATGCCTAGGCT
ATAGGAAGTACTCAAAGCTTGTTAAATTGAATTCTATCCTTCTTATTCAATTCTACACATGGAGGAAAAACTCATCA
GGGATGGAGGCACGCCTCTAAGGAAGGCAGGTGTGGCTCTGCAGTGTGATTGGGTACTTGCAGGACGAAGGGTGGGG
TGGGAGTGGCTAACCTTCCATTCCTAGTGCAGAGGTCACAGCCTAAACATCAAATTCCTTGAGGTGCGGTGGCTCAC
TCCTGTAATCACAGCAGTTTGGGACGCCAAGGTGGGCAGATCACTTGAGGTCAGGAGTTGGACACCAGCCCAGCCAA
CATAGTGAAACCTGGTCTCTGCTTAAAAATATAAAAATTAGCTGGACGTGGTGACGGGAGCCTGTAATCCAACTACT
TGGGAGGCTGAGGCAGGAGAATCGCTTGAACCGGGGAGGTGGAGTTTGCACTGAGCAGAGATCATGCCATTGCACTC
CAGCCTCCAGAGCGAGACTCTGTCTAAAGAAAAACGAAAACAAACAAACAAACAAACAAACAAAACCCATCAAATTC
CCTGACCGAACAGAATTCTGTCTGATTGTTCTCTGACTTATCTACCATTTTCCCTCCTTAAAGAAACTGTGGAACTT
CCTTCAGCTAGAGGGGCCTGGCTCAGAAGCCTCTGGTCAGCATCCAAGAAATACTTGATGTCACTTTGGCTAAAGGT
ATGATGTGTAGACAAGCTCCAGAGATGGTTTCTCATTTCCATATCCACCCACCCAGCTTTCCAATTTTAAAGCCAAT
TCTGAGGTAGAGACTGTGATGAACAAACACCTTGACAAAATTCAACCCAAAGACTCACTTTGCCTAGCTTCAAAATC
CTTACTCTGACATATACTCACAGCCAGAAATTAGCATGCACTAGAGTGTGCATGAGTGCAACACACACACACACCAA
TTCCATATTCTCTGTCAGAAAATCCTGTTGGTTTTTCGTGAAAGGATGTTTTCAGAGGCTGACCCCTTGCCTTCACC
TCCAATGCTACCACTCTGGTCTAAGTCACTGTCACCACCACCTAAATTATAGCTGTTGACTCATAACAATCTTCCTG
CTTCTACCACTGCCCCACTACAATTTCTTCCCAATATACTATCCAAATTAGTCTTTTCAAAATGTAAGTCATATATG
GTCACCTCTTTGTTCAAAGTCTTCTGATAGTTTCCTATATCATTTATAATAAAACCAAATCCTTACAATTCTCTACA
ATAGTTGTTCATGCATATATTATGTTTATTACAGATACGCATATATATAGCTCTCATATAAATAAATATATATATTT
ATGTGTATGTGTGTAGAGTGTTTTTTCTTACAACTCTATGATGTAGGTATTATTAGTGTCCCAAATTTTATAATTTA
GGACTTCTATGATCTCATCTTTTATTCTCCCCTTCACCGAATCTCATCCTACATTGGCCTTATTGATATTCCTTGAA
AATTCTAAGCATCTTACATCTTTAGGGTATTTACATTTGCCATTCCCTATGCCCTAAATATTTAATCATAGTTTCAT
ATAAATGGGTTCCTCATCATCTATGGGTACTCTCTCAGGTGTTAACTTTATAGTGAGGACTTTCCTGCCATACTACT
TAAAGTAGCGATACCCTTTCACCCTGTCCTAATCACACTCTGGCCTTCATTTCAGTTTTTTTTTTTTCTCCATAGCA
CCTAATCTCATTGGTATATAACATGTTTCATTTGCTTATTTAATGTCAAGCTCTTTCCACTATCAAGTCCATGAAAA
CAGGAACTTTATTCCTCTATTCTGTTTTTGTGCTGTATTCTTAGCAATTTTACAATTTTGAATGAAATGAATGAGCA
GTCAAACACATATACAACTATAATTAAAAGGATGTATGCTGACACATCCACTGCTATGCACACACAAAGAAATCAGT
GGAGTAGAGCTGGAAGCGCTAAGCCTGCATAGAGCTAGTTAGCCCTCCGCAGGCAGAGCCTTGATGGGATTACTGAG
TTCTAGAATTGGACTCATTTGTTTTGTAGGCTGAGATTTGCTCTTGAAAACTTGTTCTGACCAAAATAAAAGGCTCA
AAAGATGAATATCGAAACCAGGGTGTTTTTTACACTGGAATTTATAACTAGAGCACTCATGTTTATGTAAGCAATTA
ATTGTTTCATCAGTCAGGTAAAAGTAAAGAAAAACTGTGCCAAGGCAGGTAGCCTAATGCAATATGCCACTAAAGTA
AACATTATTCCATAGGTGTCAGATATGGCTTATTCATCCATCTTCATGGGAAGGATGGCCTTGGCCTGGACATCAGT
GTTATGTGAGGTTCAAAACACCTCTAGGCTATAAGGCAACAGAGCTCCTTTTTTTTTTTTCTGTGCTTTCCTGGCTG
TCCAAATCTCTAATGATAAGCATACTTCTATTCAATGAGAATATTCTGTAAGATTATAGTTAAGAATTGTGGGAGCC
ATTCCGTCTCTTATAGTTAAATTTGAGCTTCTTTTATGATCACTGTTTTTTTAATATGCTTTAAGTTCTGGGGTACA
TGTGCCATGGTGGTTTGCTGCACCCATCAACCCGTCATCTACATTAGGTATTTCTCCTAATGCTATCCTTCCCCTAG
CCCCCCACCCCCAACAGGCCCCAGTGTGTGATGTTCCCCTCCCTGTGTCCATGGATCACTGGTTTTTTTTTTTTTTT
TTTTTTTTTTTTTAAAGTCTCAGTTAAATTTTTGGAATGTAATTTATTTTCCTGGTATCCTAGGACCTGCAAGTTAT
CTGGTCACTTTAGCCCTCACGTTTTGATGATAATCACATATTTGTAAACACAACACACACACACACACACACACACA
TATATATATATAAAACATATATATACATAAACACACATAACATATTTATCGGGCATTTCTGAGCAACTAACTCATGC
AGGACTCTCAAACACTAACCTATAGCCTTTTCTATGTATCTACTTGTGTAGAAACCAAGCGTGGGGACTGAGAAGGC
AATAGCAGGAGCATTCTGACTCTCACTGCCTTTGGCTAGGTCCCTCCCTCATCACAGCTCAGCATAGTCCGAGCTCT
TATCTATATCCACACACAGTTTCTGACGCTGCCCAGCTATCACCATCCCAAGTCTAAAGAAAAAAATAATGGGTTTG
CCCATCTCTGTTGATTAGAAAACAAAACAAAATAAAATAAGCCCCTAAGCTCCCAGAAAACATGACTAAACCAGCAA
GAAGAAGAAAATACAATAGGTATATGAGGAGACTGGTGACACTAGTGTCTGAATGAGGCTTGAGTACAGAAAAGAGG
CTCTAGCAGCATAGTGGTTTAGAGGAGATGTTTCTTTCCTTCACAGATGCCTTAGCCTCAATAAGCTTGCGGTTGTG
GAAGTTTACTTTCAGAACAAACTCCTGTGGGGCTAGAATTATTGATGGCTAAAAGAAGCCCGGGGGAGGGAAAAATC
ATTCAGCATCCTCACCCTTAGTGACACAAAACAGAGGGGGCCTGGTTTTCCATATTTCCTCATGATGGATGATCTCG
TTAATGAAGGTGGTCTGACGAGATCATTGCTTCTTCCATTTAAGCCTTGCTCACTTGCCAATCCTCAGTTTTAACCT
TCTCCAGAGAAATACACATTTTTTATTCAGGAAACATACTATGTTATAGTTTCAATACTAAATAATCAAAGTACTGA
AGATAGCATGCATAGGCAAGAAAAAGTCCTTAGCTTTATGTTGCTGTTGTTTCAGAATTTAAAAAAGATCACCAAGT
CAAGGACTTCTCAGTTCTAGCACTAGAGGTGGAATCTTAGCATATAATCAGAGGTTTTTCAAAATTTCTAGACATGA
GATTCAAAGCCCTGCACTTAAAATAGTCTCATTTGAATTAACTCTTTATATAAATTGAAAGCACATTCTGAACTACT
TCAGAGTATTGTTTTATTTCTATGTTCTTAGTTCATAAATACATTAGGCAATGCAATTTAATTAAAAAAACCCAAGA
ATTTCTTAGAATTTTAATCATGAAAATAAATGAAGGCATCTTTACTTACTCAAGGTCCCAAAAGGTCAAAGAAACCA
GGAAAGTAAAGCTATATTTCAGCGGAAAATGGGATATTTATGAGTTTTCTAAGTTGACAGACTCAAGTTTTAACCTT
CAGTGCCCATGATGTAGGAAAGTGTGGCATAACTGGCTGATTCTGGCTTTCTACTCCTTTTTCCCATTAAAGATCCC
TCCTGCTTAATTAACATTCACAAGTAACTCTGGTTGTACTTTAGGCACAGTGGCTCCCGAGGTCAGTCACACAATAG
GATGTCTGTGCTCCAAGTTGCCAGAGAGAGAGATTACTCTTGAGAATGAGCCTCAGCCCTGGCTCAAACTCACCTGC
AAACTTCGTGAGAGATGAGGCAGAGGTACACTACGAAAGCAACAGTTAGAAGCTAAATGATGAGAACACATGGACTC
ATAGAGGGAAACAACGCATACTGGGGCCTATCAGAGGGTGGAGGGTGAGAGAAGGAGAGGATCAGGAAAAATCACTA
ATGGATGCTAAGCGTAATACCTGAGTGATGAGATCATCTATACAACAAACCCCCTTGACATTCATTTATCTATGTAA
CAAACCTGCACATCCTGTACACGTACCCCTGAACTTAAAATAAAAGTTGAAAACAAGAAAGCAACAGTTTGAACACT
TGTTATGGTCTATTCTCTCATTCTTTACAATTACACTAGAAAATAGCCACAGGCTCCTGCAAGGCAGCCACAGAATT
TATGACTTGTGATATCCAAGTCATTCCTGGATAATGCAAAATCTAACACAAAATCTAGTAGAATCATTTGCTTACAT
CTATTTTTGTTCTGAGAATATAGATTTAGATACATAATGGAAGCAGAATAATTTAAAATCTGGCTAATTTAGAATCC
TAAGCAGCTCTTTTCCTATCAGTGGTTTACAAGCCTTGTTTATATTTTTCCTATTTTAAAAATAAAAATAAAGTAAG
TTATTTGTGGTAAAGAATATTCATTAAAGTATTTATTTCTTAGATAATACCATGAAAAACATTCAGTGAAGTGAAGG
GCCTACTTTACCCAACAAGAATCTAATTTATATAATTTTTCATACTAATAGCATCTAAGAACAGTACAATATTTGAC
TCTTCAGGTTAAACATATGTCATAAATTAGCCAGAAAGATTTAAGAAAATATTGGATGTTTCCTTGTTTAAATTAGG
CATCTTACAGTTTTTAGAATCCTGCATAGAACTTAAGAAATTACAAATGCTAAAGCAAACCCAAACAGGCAGGAATT
AATCTTCATCGAATTTGGGTGTTTCTTTCTAAAAGTCCTTTATACTTAAATGTCTTAAGACATACATAGATTTTATT
TTACTAATTTTAATTATACAGACAATAAATGAATATTCTTACTGATTACTTTTTCTGACTGTCTAATCTTTCTGATC
TATCCTGGATGGCCATAACACTTATCTCTCTGAACTTTGGGCTTTTAATATAGGAAAGAAAAGCAATAATCCATTTT
TCATGGTATCTCATATGATAAACAAATAAAATGCTTAAAAATGAGCAGGTGAAGCAATTTATCTTGAACCAACAAGC
ATCGAAGCAATAATGAGACTGCCCGCAGCCTACCTGACTTCTGAGTCAGGATTTATAAGCCTTGTTACTGAGACACA
AACCTGGGCCTTTCAATGCTATAACCTTTCTTGAAGCTCCTCCCTACCACCTTTAGCCATAAGGAAACATGGAATGG
GTCAGATCCCTGGATGCAAGCCAGGTCTGGAACCATAGGCAGTAAGGAGAGAAGAAAATGTGGGCTCTGCAACTGGC
TCCGAGGGAGCAGGAGAGAATCAACCCCATACTCTGAATCTAAGAGAAGACTGGTGTCCATACTCTGAATGGGAAGA
ATGATGGGATTACCCATAGGGCTTGTTTTAGGGAGAAACCTGTTCTCCAAACTCTTGGCCTTGAGATACCTGGTCCT
TATTCCTTGGACTTTGGCAATGTCTGACCCTCACATTCAAGTTCTGAGGAAGGGCCACTGCCTTCATACTGTGGATC
TGTAGCAAATTCCCCCTGAAAACCCAGAGCTGTATCTTAATTGTTTAAAAAAATTATATTATCTCAAGGACTGTTCT
TCTCTGAGTAGCCAAGCTCAGCTTGGTTCAAGCTACAAGCAGCTGCGCTGCTTTTTGTCTAGTCATTGTTCTTTTAT
TTCAGTGGATCAAATACGTTCTTTCCAAACCTAGGATCTTGTCTTCCTGGACTATATATTTTATCCACGAAGTCTTA
ATCTGGGGTCCACAGAACACTAGGGGGCTGGTGAAGTTTATAGAAAAAAAATCTGTATTTTTACTTACATGTAACTG
AAATTTAGCATTTTCTTCTACTTTGAATGCAAAGGACAAACTAGAATGACATCATCAGTACCTATTGCATAGTTATA
AAGAGAAACCACAGATATTTTCATACTACACCATAGGTATTGCAGATCTTTTTGTTTTTGTTTTTGTTTGAGATGGA
GTTTCGCTCTTATTGCCCAGGCTGGAGTGCAGTGGCATGATTTCGGCTCACTGCAACCTCCCCTTCCTGCATTCAAG
CAATTCTCCTGCCTTGGCCTCCAGAGTAGCTGGGGATTACAGGCACCTGCCACCATGCCAGTCTAATTTTTGTATTT
TTAGTAGAGAATGGGTTTCGCCATGTTGGCCAGGCTGGTCTTGAACTCCTGACCTCAGATGATCTGCCCGCCTTGGC
CTCCTGAAGTGCTGGGATTATAGGTGTGAGCCACCACGCCTGGCCCATTGCAGATATTTTTAATTCACATTTATCTG
CATCACTACTTGGATCTTAAGGTAGCTGCAGACCCAATCCCAGATCTAATGCTTTCATAAAGAAGCAAATATAATAA
ATACTATACCACAAATGTAATGTTTGATGTCTGATAATGATATTTCAGTGTAATTAAACTTAGCACTCCATGTATAT
TATTTGATGCAATAAAAACATATTTTTTTAGCACTTACAGTCTGCCAAACTGGCCTGTGACACAAAAAAAGTTTAGG
GGAATTCCCCTAGTTTTGTCTGTGTTAGCCAATGGTTAGAATATATGCTCAGAAAGATACCATTGGTTAATAGCTAA
AAGAAAATGGAGTAGAAATTCAGTGGCCTGGAATAATAACAATTTGGGCAGTCATTAAGTCAGGTGAAGACTTCTGG
AATCATGGGAGAAAAGCAAGGGAGACATTCTTACTTGCCACAAGTGTTTTTTTTTTTTTTTTTTTTTATCACAAACA
TAAGAAAATATAATAAATAACAAAGTCAGGTTATAGAAGAGAGAAACGCTCTTAGTAAACTTGGAATATGGAATCCC
CAAAGGCACTTGACTTGGGAGACAGGAGCCATACTGCTAAGTGAAAAAGACGAAGAACCTCTAGGGCCTGAACATAC
AGGAAATTGTAGGAACAGAAATTCCTAGATCTGGTGGGGCAAGGGGAGCCATAGGAGAAAGAAATGGTAGAAATGGA
TGGAGACGGAGGCAGAGGTGGGCAGATCATGAGGTCAAGAGATCGAGACCATCCTGGCAAACATGGTGAAATCCCGT
CTCTACTAAAAATAAAAAAATTAGCTGGGCATGGTGGCATGCGCCTGTAGTCCCAGCTGCTCGGGAGGCTGAGGCAG
GAGAATCGTTTGAACCCAGGAGGCGAAGGTTGCAGTGAGCTGAGATAGTGCCATTGCACTCCAGTCTGGCAACAGAG
TGAGACTCCGTCTCAAAAAAAAAAAAAAGAAAGAAAGAAAAGAAAAAGAAAAAAGAAAAAATAAATGGATGTAGAAC
AAGCCAGAAGGAGGAACTGGGCTGGGGCAATGAGATTATGGTGATGTAAGGGACTTTTATAGAATTAACAATGCTGG
AATTTGTGGAACTCTGCTTCTATTATTCCCCCAATCATTACTTCTGTCACATTGATAGTTAAATAATTTCTGTGAAT
TTATTCCTTGANTCCCAAAATATTGAGGTAAATAACAATGGTATTATAAAAGGGCAGATTAAGTGATATAGCATAAG
CAATATTCTTCAGGCACATGGATCGAATTGAATACACTGTAAATCCCAACTTCCAGTTTCAGCTCTACCAAGTAAAG
AGCTAGCAAGTCATCAAAATGGGGACATACAGAAAAAAAAAAGGACACTAGAGGAATAATATACCCTGACTCCTAGC
CTGATTAATATATCGATTCACTTTTACTCTGTTTGGTGACAAATTCTGGCTTTAAATAATTTTAGGATTTTAGGCTT
CTCAGCTCCCTTCCCAGTGAGAAGTATAAGCAGGACAGCAGGCAAGCAAGAAGAGAGCCCAAGGCAATACTCACAAA
GTAGCCAGTGTCCCCTGTGGTCATAGAGAAATGGAAAGAGAGAGGANTCCCCCCTTGGAGCCACTGGGTGGTAATCC
TTTCCGTCCGTTCCTCTCTAGGGAATCACCCCAAGGTACTGTACTTTGGGATTAAGGCTTTAGTCCCACTGTGGACT
ACTTGCTATTCTGTTCAGTTTCTGAAGGAACTATGTACGGTTTTTGTCTCCCTAGAGAAACTAAGGTACAGAAGTTT
TGTTTACAATGCACTCCTTAAGAGAGCTAGAACTGGGTGAAGANTCCTGGTTTAACCAGCCTTAATTTCCTTTCCCT
GGGCCCCGGTTTGGTCACGTCACTGTCACCACCTTTAAGGCAAATGTTAAATGCGCTTTGGCTGAACTTTTTCCTAT
TTTGAGATTTGCTCCTTTATATGAGGCTTTCTTGGAAAAGGAGAATGGGAGAGATGGATATCATTTTGGAAGATGAT
GAAGAGGGTAAAAAAGGGTACAAATGGAAATTTGTGTTGCAGATAGTATGAGGAGCCAACAAAAAAGAGCCTCAGGA
TCCAGCACACATTATCACAAACTTAGTGTCCATCCATCACTGCTGACCCTCTCCGGACCTGACTCCACCCCTGAGGA
CACAGGTCAGCCTTGACCAATGACTTTTAAGTACCATGGAGAACAGGGGGCCAGAACTTCGGCAGTAAAGAATAAAA
GGCCAGACAGAGAGGCAGCAGCACATATCTGCTTCCGACACAGCTGCAATCACTAGCAAGCTCTCAGGCCTGGCATC
ATGGTGCATTTTACTGCTGAGGAGAAGGCTGCCGTCACTAGCCTGTGGAGCAAGATGAATGTGGAAGAGGCTGGAGG
TGAAGCCTTGGGCAGGTAAGCATTGGTTCTCAATGCATGGGAATGAAGGGTGAATATTACCCTAGCAAGTTGATTGG
GAAAGTCCTCAAGATTTTTTGCATCTCTAATTTTGTATCTGATATGGTGTCATTTCATAGACTCCTCGTTGTTTACC
CCTGGACCCAGAGATTTTTTGACAGCTTTGGAAACCTGTCGTCTCCCTCTGCCATCCTGGGCAACCCCAAGGTCAAG
GCCCATGGCAAGAAGGTGCTGACTTCCTTTGGAGATGCTATTAAAAACATGGACAACCTCAAGCCCGCCTTTGCTAA
GCTGAGTGAGCTGCACTGTGACAAGCTGCATGTGGATCCTGAGAACTTCAAGGTGAGTTCAGGTGCTGGTGATGTGA
TTTTTTGGCTTTATATTTTGACATTAATTGAAGCTCATAATCTTATTGGAAAGACCAACAAAGATCTCAGAAATCAT
GGGTCGAGCTTGATGTTAGAACAGCAGACTTCTAGTGAGCATAACCAAAACTTACATGATTCAGAACTAGTGACAGT
AAAGGACTACTAACAGCCTGAATTGGCTTAACTTTTCAGGAAATCTTGCCAGAACTTGATGTGTTTATCCCAGAGAA
TTGTATTATAGAATTGTAGACTTGTGAAAGAAGAATGAAATTTGGCTTTTGGTAGATGAAAGTCCATTTCAAGGAAA
TAGAAATGCCTTATTTTATGTGGGTCATGATAATTGAGGTTTAGAAGAGATTTTTGCAAAAAAAATAAAAGATTTGC
TCAAAGAAAAATAAGACACATTTTCTAAAATATGTTAAATTTCCCATCAGTATTGTGACCAAGTGAAGGCTTGTTTC
CGAATTTGTTGGGGATTTTAAACTCCCGCTGAGAACTCTTGCAGCACTCACATTCTACATTTACAAAAATTAGACAA
TTGCTTAAAGAAAAACAGGGAGAGAGGGAACCCAATAATACTGGTAAAATGGGGAAGGGGGTGAGGGTGTAGGTAGG
TAGAATGTTGAATGTAGGGCTCATAGAATAAAATTGAACCTAAGCTCATCTGAATTTTTTGGGTGGGCACAAACCTT
GGAACAGTTTGAGGTCAGGGTTGTCTAGGAATGTAGGTATAAAGCCGTTTTTGTTTGTTTGTTTGTTTTTTCATCAA
GTTGTTTTCGGAAACTTCTACTCAACATGCCTGTGTGTTATTTTGTCTTTTGCCTAACAGCTCCTGGGTAACGTGAT
GGTGATTATTCTGGCTACTCACTTTGGCAAGGAGTTCACCCCTGAAGTGCAGGCTGCCTGGCAGAAGCTGGTGTCTG
CTGTCGCCATTGCCCTGGCCCATAAGTACCACTGAGTTCTCTTCCAGTTTGCAGGTCTTCCTGTGACCCTGACACCC
TCCTTCTGCACATGGGGACTGGGCTTGGCCTTGAGAGAAAGCCTTCTGTTTAATAAAGTACATTTTCTTCAGTAATC
AAAAATTGCAATTTTATCTTCTCCATCTTTTACTCTTGTGTTAAAAGGAAAAAGTGTTCATGGGCTGAGGGATGGAG
AGAAACATAGGAAGAACCAAGAGCTTCCTTAAGAAATGTATGGGGGCTTGTAAAATTAATGTGGATGTTATGGGAGA
ATTCCCAAGATTCCCAAGGAGGATGATATGATGGAGAAAAATCTTTATCGGGGTGGGAAAATGGTTAATTAAGTGGC
AGAGACTCCTAGGCAGTTTTTACTGCACCGGGGAAAGAAGGAGCTGTTGTGGTACCTGAGAAAGCAGATTTGTGGTA
CATGTCACTTTTCATTAAAAACAAAAACAAAACAAAACAAAACTTCATAGATATCCAAGATATAGGCTGAGAATTAC
TATTTTAATTTACTCTTATTTACATTTTGAAGTAGCTAGCTTGTCACATGTTTTATGAAATTGATTTGGAGATAAGA
TGAGTGTGTATCAACAATAGCCTGCTCTTTCCATGAAGGATTCCATTATTTCATGGGTTAGCTGAAGCTAAGACACA
TGATATCATTGTGCATTATCTTCTGATACAATGTAACATGCACTAAAATAAAGTTAGAGTTAGGACCTGAGTGGGAA
AGTTTTTGGAGAGTGTGATGAAGACTTTCCGTGGGAGATAGAATACTAATAAAGGCTTAAATTCTAAAACCAGCAAG
CTAGGGCTTCGTGACTTGCATGAAACTGGCTCTCTGGAAGTAGAAGGGAGAGTAAGACATACGTAGAGGACTAGGAA
AGACCAGATAGTACAGGGCCTGGCTACAAAAATACAAGCTTTTACTATGCTATTGCAATACTAAACGATAAGCATTA
GGATGTTAAGTGACTCAGGAAATAAGATTTTGGGAAAAAGTAATCTGCTTATGTGCACAAAATGGATTCAAGTTTGC
AGATAAAATAAAATATGGATGATGATTCAAGGGGACAGATACAATGGTTCAAACCCAAGAGGAGCAGTGAGTCTGTG
GAATTTTGAAGGATGGACAAAGGTGGGGTGAGAAAGACATAGTATTCGACCTGACTGTGGGAGATGAGAAGGAAGAA
GGAGGTGATAAATGACTGAAAGCTCCCAGACTGGTGAAGATAACAGGAGGAAACCATGCACTTGACCCTGGTGACTC
TCATGTGTGAAGGGTAGAGGGATATTAACAGATTTACTTTTTAGGAAGTGCTAGATTGGTCAGGGAGTTTTGACCTT
CAGGTCTTGTGTCTTTCATATCAAGGAACCTTTGCATTTTCCAAGTTAGAGTGCCATATTTTGGCAAATATAACTTT
ATTAGTAATTTTATAGTGCTCTCACATTGATCAGACTTTTTCCTGTGAATTACTTTTGAATTTGGCTGTATATATCC
AGAATATGGGAGAGAGACAAATAATTATTGTAGTTGCAGGCTATCAACAATACTGGTCTCTCTGAGCCTTATAACCT
TTCAATATGCCCCATAAACAGAGTAAACAGGGATTATTCATGGCACTAAATATTTTCACCTAGGTCAGTCAACAAAT
GGAGGCAATGTGCATTTTTTGATACATATTTTTATATATTTATGGGGCATGTGATACTTACATGCCTAGAACATGTG
ACTGATTAAGTCTAGATATTTAGGATATCCATTACTTTGAGCATTTATCATTTCTATGTATTGAGAAAATTTCAAAT
CCTCATTTCTGACCATTTTGAAATATATAATAAATAGTAATTAACTATAGTCACCCTACTCAAATATCAACATTATA
AACTAACTAATCCTTCTTTCCACTTTTTTACCAACCAACATCTCTTAAATCCCCTGCCATACACATCACACATTTTT
CAGCTCTGATAACTATCATTCTACTCTCATACCACCATGAGACCACTTTTTTAGCTCCACAGATGAATAAAAACATG
TGATATTTGACTTTCTGTATCTGGCTTATTTTATTATCTATCTCTTTGGCATACCAAGAGTTTGTTTTTGTTCTGCT
TCAGGGCTTTCAATTAACATAATGACCTCTGGTTCCATCCATGTTGCTACAAATGACAAGATTTCATTCTTTTTCAT
GGCAAAATAGTACTGTGCAAAAAATACAATTTTTTAATCCGTTCATCTGTTGATAGACACTTAGGTTGATCCCAAAC
CTTAACTATTGTGAATAGGTGCTTCAATAAACATGAGTGTAATGTGTCCATTGGATATACTGATTTCCTTTCTTTTG
GATAAATAACCACTAGTGAGATTGCTGGATTGTATGATAGTTCTGTTTTTAGTTTATTGAGAAATCTTCATACTGTT
TTCCATAATGGTTGTACTATTTTACATTCCCACCAACAGTGTGTAAGAAAGAGTTCCCTTTTCTCCATATCCTCACA
AGGATCTGTTATTTTTTGTCTTTTTTGTTAATAGCATTTTAACTAGAGTAAGTAGATATCTCATTGTAGTTTTGATT
TGCATTTCCCTGATCATTAGTGATGTTGAGATTTTTTCATATGTTTGTTGGTCATTTGTATATCTTTTTCTGAGATT
GTCTGTTCATGTCCTTATCCTACTTTTATTGGGATTGTTGTTATTTTCTTGATAATCATTGTGTCATTTTAGAGCCT
GGATATTATTCTTTTGTCAGATGTATAGATTGTGAAGATTTTCTCCTCTGTGGGTTGTCTGTTTATTCTGCAGACTC
TTCCTTTTGCCATGCAAAAGCTCTTTAGTTTAATTTAGTCCCAGATATTTTCTTTGTTTTTATGTGTTTGCATTTGT
GTTCTTGTCATGAAATCCTTTCCTAAGCCAATGTGTAGAAGGGTTTTTCCGATGTTATTTTCTAGAATTGTTACAGT
TTCAGGCTTAGATTTAAGTCCTTGATCCATCTTAAGTTGATTTTTGTATAAGGTGAGAGATGAAGATCCAGTTTCAT
TCTCCTACATGTAGCTTGCCAGCTATCCCGACTCATTTGTTGAATAGGGTGCCCTTTCCCATTTATGTTTTTGTTTG
CTTTGTCAAAGATCAGTTCGGATGTAAGTATTTGAGTTTATTTCTGGGTTCTCTATTCTGTTCCATTGGTCCGATGT
GCCTATTTGTACACCAGCATCATGCTGTGTTTTTGGTGACTATGGCCTTATTGTATAGTTTGAAATGAGGTAATGTA
ATGCCATTCAGATTTGTTCTTTTTTTTAGACTTGCTTGTTTATTGGGCTCTTTTTTGGTTCCATAAGAATTTTAGGA
TTGTTTTTTCTAGTTCTGTGAAGGCTAATGGTGGTATTTATGGGAATTGCAATGCAATTTGTAGGTTGCTTCTGGCA
TTATGGCCATTTTCACAATATTGATTCTACCCATCTATGAGAATGGCATGTGTTTCCATTTGTTTGTGTCTTATATG
ATTACTATCAGCCGTGTTTTGTAGTTTTCCTTGTAGATGTCTTTCACCTCCTTGGTTAGGTATATATTCCTAAGTTT
TTGTTTTGTTTTGTTTTGTTTTTTGCAGCTATTGTAAAAGGGGTTGAGTTATTGATTTTATTCTCATCTTGGTCATT
GCTGGTATGTAAGAAAGCAACTCATTGGTGTACGTTAATTTTGTATCCAGAAACTTTGCTGAATTATTTTATCAGTT
CTAGGGGGTTTTGGAGGAGTCTTTAGAGTTTTCTACATACACAATCATATCATCAGCAAACAGTGACAGTTTGACTT
TCTCTTTAACAATTTGGATGTGCTTTACTTGTTTCTCTTGTCTGATTGCTCTTGCTAGGACTTCCAGTAATATGTTA
AAGAGAAGTGGTGAGAGTGGGTATCCTTGTCTCATTCCAGTTTTCAGACAGAATGCTTTTAACTTTTTCCCATTCAA
TATAATGTTGGCTGTGTGTTTACCATAGCTGGCTTTTATTACATTGAGGTATGTCCTTTGTAAACCGATTTTGCTGA
GTTTTAGTCATAAAGTGATGTTGAATTTTGTTGAATGCAGTTTCTGTGGCTATTGAGATAATCACATGATTTTTGTT
TCCAATTCTCTTTATGTTGTGTATCACACTTATTGACTTGCGTATGTTAAACCATCCGTGCATCCCTCGCATGAAAC
CACTTGATCATGGGTTTTGATATGCCGTGTGGGATGCTATTAGCTATATTTTGTCAAGGATGTTGGCATCTATGTTC
ATCAGGGATATTGATCTGTAGTGTTTTTTTTTTTTGGTTATGTTCTTTCCCAGTTTTGGTATTAAGGTGATACTGGC
TTCATAGAATGATTTAGGGAGGATTCTCTCTTTCTCTATCTTGTAGAATACTGTCAATAGGATTGGTATCAATTCTT
CTTTGAATGTCTGGTAGAATTCGAACGTCTCCTTTAGGTTTTCTAGTTTATTCATGTAAAGGTGTTCATAGTAACCT
TGAATAATCTTTTGTATTTCTGTGGTATCAGTAATAGTATCTCCTGTTTTGTTTCTAACTGAGTTTATTTGCACTTC
TCTCCTCTTTTCTTGGTTAATCTTGCTAATGGTCTATCAGTTTTATTTATCTTTTCAAAGAACCAGCTTTTTATTTC
ATTTAGCTTTTGTATTTTTTTGCAGTTGTTTTAATTTCATTTAGTTCTCCTCTTATCTTAGTTATTCCCTTTCTTTT
GCTGGGTTTTGGTTCTGTTTGTTTTTGTTTCTCTAGTTTCTTGTGGTGTGACCTTATATTGTCTGTCCTCTTTCAGA
CTCTTTGACATCGACATTTAGGGCTGTGAACTTTCCTTTTAGCACCATCTTTGCTGTATCCTAGAGGTTTTGATAGG
TGTGTCACTATTGTCGGTCAGTTCAAGTAATTTTGTTGTTCTTATTATACTTTAAGTTCTGGGATACATGTGCAGAA
TGTGCAGGTTTGTTACATAGGTATAGATGTGCCATGGTGGTTTGCTGCTCCCATCAACCTGTCATCTACATTAGGTA
TTTCTTTTAATGTTATCCCTCTCCTAACCCCCTCACCCCCCGACAGGCCCTGGTGTGTGATGTTCCCCTCCCTGTGT
CCATGTGTTCTCATTGTTCAACTCCCACTTATGAGTGAGAACGTGTGGTGTTTGGTTTCTCTGTTCCTGTGTTAGTT
TGCTCAGAATGATGTTTCCACCTTCACCATGTCCCTGCAAAGACATGAACTCATCATTTTATGGCTGCATATATTCC
ATGGTGTATATGTGCCACATTTTCTTTATCCATTATATCGCTGATGGCCATTTGGGTTGGTTCCAAGTCTTTGGTAT
TGTGAATAGTGCCGCAATAAACATACGTGTGCACATGTCTTTATAGTAGAATGATTTCTAATTCTTTGGGTATATAC
CCAGTAATGGGATTGCTGGGTCAAACAGTATTTCTGGTTCTAGATCCTTGAGGAATTGCCACACTGTCTTCCACAAT
GGTTGAACTAATTTACACACCCATCAACAGTGTAAAATTTTTCCTATTCTTCCACATCCTCTCCAGCACCTTTTGTT
TCCTGACTTTTTAATAATTGCCATTCTAACTGGCATGAGATGGTATCTCATTGTGGTTTTGATTTGCATTTCTCTAA
TGACCAGTGATGATGAGCTTCTTTTCATGTGTTTCTTGGCCACATAAATGACTTCTTTAGAGAAGCATCTGTTCATA
TCCTTTGTCCACTTTTTGATGGGGTCGTTAGGTTTTTTCTTGTAAATTTGTTGAAGTTCTTTGTAGATTTTGGATGT
TAGCCCTTTGTCAGATGGATAGATTGGCAAAAATTTTCTCCCATTCTGTAGGTTGCCTGTTCACTCTGATGATAGTC
TTTTGCTGTGCAGAAGCTCTTTAGTTTAATTAGATCCCATATGTCAATTTTGGCCTTTGTTGTCATTGCTTTTGATG
TTTAGTCGTGGAATTTTGCCCATGCCTATGTCCTGAATGGTATTGCCTAGGTTATCTTCTAGGATTTTTATGGTTTT
AGGTTGCACATTTAAGTCTTTAATCCACCTTGAGTTAATTTTTGTATAAGGTGTAAGGAAGGGGTACAGTTTCAGTT
TTATGCATATTGCTAGCCAGTTTTTCCAGCACCATTTATTAAATAGGGAATTCTTTCTCCATTGCTTTTGTGATGTT
TGTCAAAGATCAGATGGTCGTAGATGTGTGGCATTATTTCTGAGGCTTCTGTTCTGTTCCACTGGTCTATATATCTG
TTTTGGTACCAGTACCATGCTGTTTTTGTTACTGTAGCCTTGTAGTATAGCTTGAAGTCAGGTAGCATCATGCCTCC
AGCTTTGTTCTTTTTGTTTAGGATTGTCTTGGCTATATGGGCTCTTTTTTGATTCCATATGACATTTAAAGTAGTTT
TTTCTAATTCTTTGAAAAAAGTCAGTGGTAGCTTGATGGGGATAGCATTGAATCTATAAATTACTTTGGGCAGTATG
GCCATTTTAAAGATATTGATTCTTTCTATCTATGAGCATGGAATGTTTTTCCATTTGTTTGTGTCCTCTCTTATTTC
CTTGAGCAGTGAGTGGTTTGTAGCTCTCCTTGAAGAGGTTCTTCACATCCCTTATAAGTTGTATTTCTAGGTATTTT
ATTTTATTCTCTTTGCAGCAATTGTGAATGGGAGTTCACCCATGATTTGGCTCTCTGCTTGTCTATTATTGGTGTAT
AGGAATGCTTGTGATTTTTGCACACTGATTTTGTATCTTGAGACTTTGCTGAAGCTGTTTATCAGCTTAAGATTTTG
GGCTGAGATGACAGGGTCTTCTAAATATACAATCATGTCATCTGCAAACAGAGACAATTTGACTTCCTCTCTTCCTA
TTTGAATATGCTTTATTTCTTTCTCTTGCCTGATTGTCCTGGCGAGAACTTCCAATACTATGTTGAGTAAGAGTGGC
GAGAGGGCATCCTTGTCTTGTGCCGGTTTTCAAAGCAAATGATTTTTAAATTTCCGTCTTGATTTCATTGTTGACCC
AATGATCATTCAGGAGCAGGTTATTTAATTTCCCTGTATTTGCATGGTTTTGAAGGTTCCTTTTGTAGTTGATTTCC
AATTTTATTCTACTGTGGTCTGAGAGAGTGCTTGATATAATTTCAATTTTTAAAAATTTATTGAGGCTTGTTTTGTG
GCATATCATATGGCCTATCTTGGAGAAAGTTCCATGTGCTGATGAATAGAATGTGTATTCTGCAGTTGTTGGGTAGA
ATGTCCTGTAAATATCTGTTAAGTCCATTTGTTCTTTAAATCCATTGTTTCTTTGTAGACTGTCTTGATGACCTGCC
TAGTGCAGTCAGTGGAGTATTGAAGTCCCCCACTATTATTATGTTGCTGTCTAGTAGTAATTGTTTTATAAATTTGG
GATCTCCAGTATTAGATGCATATATATTAAGAATTGTAATATTCTCCCATTGGACAAGGGCTTTTATCATTATATGA
TGTCCCTCTTTGTCTTTTTTAACTGCTGTTTCTTTAAAGTTTGTTTTGTCTGACATAAGAATAGCTGCTTTGGCTCG
CTTTTGGTGTCCATTTGTGTGGAATGTCATTTTCCACCCCTTTACCTTAAGTTTATGTGAGTCCTTATGTGTTAGGT
GAGTCTCCTGAAGGCGGCAGATAACTGGTTGGTGAATTCTATTCATTCTGCAATTCTGTATCTTTTAAGTGGAGCAT
TTAGTCCATTTACATTCAACATCAGTATTGAGGTGTGAGGTGACTATTCCATTCTTCGTGGTATTTGTTGCCTGTGT
ATCTTTTTATCTGTATTTTTGTTGTATATGTCCTATGGGATTTATGCTTTAAAGAGGTTCTGTTTTGATGTGCTTCC
AGGGTTTATTTCAAGATTTAGAGCTCCTTTTATCATTCTTGTAGTGTTGGCTTGGTAGTGCCGAATTCTCTCAGCAT
TTGTTTTTCTGAAAAACACTGTGTATTTTCTTCATTTGTGAAGCTTAGTTTCACTGGATATAAAATTCTTGGCTGAT
AATTGTTTTGTTTAAGAAGGCTGAAGATAGGGCCATATTCACTTCTAGCTTTTACGGTTTCTGCTGAGAAATCTGCT
GTTAATCTGATAGGTTTTCTTTCATAGGTTACCTGGTAGTTTCACCTCACAGCTCTTAAGATTCTCTTTGTCTTTAG
ATAACTTTGGATACTCTGATGACAATGTACCTAGGCAATGATATTTTTGCAATGAATTTCCCAGGTGTTTATTGAGC
TTCTTTGTATTTGGATATCTAGGTCTCTAGCAAGGAGGGGGAAGTTTTCCTTGATTATTTCCATGGACAAGTTTTCC
AAACTTTTAGATTTCTCTTCTTTCTCAGGAATGCTGATTATTCTTAGGTTTGATTGTTTAACATAATCCCAGATTTC
TTGGAGGCTTTGTTCATATTTTCTTATTCTTTTTTCTTTGTCTTTGTTGGATTGGGTAATTCAAAAACTTTGTCTTC
AAGCTCTGAATTTCTTCTGCTTGGATTCTATTGCTGAGACTTTCTAGAGCATTTTGCATTTCTATAAGTGCATCCAT
TCATCCATTGTTTCCTGAAGTTTTGAATGTTTTTTATTTATGCTATCTCTTTAACTGAAGATTTCTCCCCTCATTTC
TTGTATCATATTTTTGGTTTTTTTAAAATTGGACTTCACCTTCCTCGGATGCCTCCTTGATTAGCTTAATAACTGAC
CTTCTGAATTATTTTTCAGGTAAATCAGGGATTTCTTCTTGGTTTGGATGCATTGCTGGTGAGCTAGTATGATTTTT
TGGGGGGTGTTAAAGAACCTTGTTTTTCATATTACCAGAGTTAGTTTTCTGGTTCCTTCTCACTTGGGTAGGCTCTG
TCAGAGGGAAAGTCTAGGCCTCAAGGCTGAGACTTTTGTCCCAGCAGGTGTTCCCTTGATGTAGCACAGTCCCCCTT
TTCCTAGGACGTGGGGCTTCCTGAGAGCCGAACTGTAGTGATTGTTATCTCTCTTCTGGATCTAGCCACCCATCAGG
TCTACCAGACTCCAGGCTGGTACTGGGGTTTGTCTGCACAGAGTCTTGTGACGTGAACCATCTGTGGGTCTCTCAGC
CATAGATACAACCACCTGCTCCAATGGAGGTGGTAGAGGATGAAATGAACTCTGTGAGGGTCCTTACTTTTGGTTGT
TCAATGCACTATCTTTTTGTGCTGGTTGGCCTCCTGCCAGGAGGTGGCACTTTCTAGAAAGCATCAGCAGAGGCAGT
CAGGTGGTGGTGGCTGGGGGGGCTGGGGCACTAGAACTCCCAAGAATATATGCCCTTTGTCTTCAGCTACTAGGGTG
AGTAAGGAAGGACCATCAGGTGGGGGCAGGACTAGTCGTGTCTGAGCTCAGAGTCTCCTTGGGCAGGTCTTTCTGTG
GCTACTGTGGGAGGATGGGGGTGTAGTTTCCAGGTCAATGGATTTATGTTCCTAGGACAATTATGGCTGCCTCTGCT
GTGTCATGCAGGTCATCAGGAAAGTGGGGGAAAGCAAGCAGTCACGTGACTTGCCCAGCTCCCATGCAACTCAAAAG
GTTGGTCTCACTTCCAGCGTGCACCCTCCCCCGCAACAGCTCCGAATCTGTTTCCATGCAGTCAGTGAGCAAGGCTG
AGAACTTGCCCAGGCTACCAGCTGCGAAACCAAGTAGGGCTGTCCTACTTCCCTGCCAGTGGAGTCTGCACACCAAA
TTCATGTCCCCCCACCAACCCCCCCACTGCCCAGCCCCTAGATCTGGCCAGGTGGAGATTTTCTTTTTCCTGTCTCT
TTTCCCAGTTCCTCTGGCAGCCCTCCCAAATGACCCCTGTGAGGCAAGGCAGAAATGGCTTCCTAGGGGACCCAGAG
AGCCCACAGGGCTTTTCCCGCTGCTTCCTCTACCCCTGTATTTTGCTTGGCCCTCTAAATTGACTCAGCTCCAGGTA
AGGTCAGAATCTTCTCCTGTGGTCTAGATCTTCAGGTTCCCAGTGAGGATGTGTGTTTGGGGGTAGACGGTCCCCCT
TTTCCACTTCCACAGTTTGGGCACTCACAATATTTGGGGTGTTTCCCGGGTCCTACATGAGCAATCTGCTTCTTTCA
GAGGGTGTGTGCGTTCTCTCAGCTTTCTTGAATTTATTTCTGCAGGTGGTTCTGCAAAAAAAATTCCTGATGGGAGA
CTTCACATGCTGCTCTGTGCATCCGAGTGGGAGCTGCAATGTACTTCTGCTGCCACCCATCTGCCATCACCCTCTAA
TTTGTCGGTAATATGCATTTTTAATCAATCTTTTTTTCTCTCTCTCTCTTTTCTTCTCCCCCAAAACTATACTGCCC
TTTGATATCAAGGAATCAAGGCCGTGATGTTGAGGGGTGGGCAGTGGATACACTCTTTACCCCTTAGGGAGCATATC
TAGATTTAGATATTGCCAATTCAAGATAACTTAATTGAAAGCAAATTCATAATGAATACACACACACACACACACAT
CTGCATGACAAGATTTTTAATAGTTGAAAGAATAACTAATAATTGTCCACAGGCAATAAGGGCTTTTTAAGCAAAAC
AGTTGTGATAAAACAGGTCATTCTTAGAATAGTAATCCAGCCAATAGTACAGGTTGCTTAGAGATTATGACATTACC
AGAGTTAAAATTCAATAATGGCTTCTCACTCCCTACCACTGAGGACAAGTTTATGTCCTTAGGTTTATGCTTCCCTG
AAACAATACCACCTGCTATTCTCCACTTTACATATCAACGGCACTGGTTCTTTATCTAACTCTCTGGCACAGCAGGA
GTTTGTTTTCTTCTGCTTCAGAGCTTTGAATTTACTATTTCAGCTTCTAAACTTTATTTGCAATGCCTTCCCATGGC
AGACTCCTTCTGTCATTTTGCCTCTGTTCGAAAACTTTTTCCTTAATTTCATTCTTAGTTAATAATATCTGAAATTA
TTTTGTTGTTTAACTTAATTATTAATTTTATGTATGTTCTACCTAGATATAATCTTCTAGAGGATTGTTTTATTCTC
TGACTTATTTAACTTAAATGCCCACTACCTTTAAAAATTATGACATTTATTTAACAGATATTTGCTGAACAAATGTT
TGAAAATACATGGGAAAGAATGCTTGAAAACACTTGAAATTGCTTGTGTAAAGAAACAGTTTTATCAGTTAGGATTT
AATCAATGTCAGAAGCAATGATATAGGAAAAATCGAGGAATAAGACAGTTATGGATAAGGAGAAATCAACAAACTCT
TAAAAGATATTGCCTCAAAAGCATAAGAGGAAATAAGGGTTTATACATGACTTTTAGAACACTGCCTGGGTTTTTGG
ATAAATGGGGAAGTTGTTGGAAAACAGGAGGGATCCTAGATATTCCTTAGTCTGAGGAGGAGCAATTAAGATTCACT
TGTTTAGAGGCTGGGAGTGGTGGCTCACGCCTGTAATCCCAGAATTTTGGGAGGCCAAGGCAGGCAGATCACCTGAG
GTCAAGAGTTCAAGACCAACCTGGCCAACATGGTGAAATCCCATCTCTACAAAAATACAAAAATTAGACAGGCATGA
TGGCAAGTGCCTGTAATCCCAGCTACTTGGGAGGCTGAGGAAGGAGAATTGCTTGAACCTGGAAGGCAGGAGTTGCA
GTGAGCCGAGATCATACCACTGCACTCCAGCCTGGGTGACAGAACAAGACTCTGTCTCAAAAAAAAAAAAGAGAGAT
TCAAAAGATTCACTTGTTTAGGCCTTAGCGGGCTTAGACACCAGTCTCTGACACATTCTTAAAGGTCAGGCTCTACA
AATGGAACCCAACCAGACTCTCAGATATGGCCAAAGATCTATACACACCCATCTCACAGATCCCCTATCTTAAAGAG
ACCCTAATTTGGGTTCACCTCAGTCTCTATAATCTGTACCAGCATACCAATAAAAATCTTTCTCACCCATCCTTAGA
TTGAGAGAAGTCACTTATTATTATGTGAGTAACTGGAAGATACTGATAAGTTGACAAATCTTTTTCTTTCCTTTCTT
ATTCAACTTTTATTTTAACTTCCAAAGAACAAGTGCAATATGTGCAGCTTTGTTGCGCAGGTCAACATGTATCTTTC
TGGTCTTTTAGCCGCCTAACACTTTGAGCAGATATAAGCCTTACACAGGATTATGAAGTCTGAAAGGATTCCACCAA
TATTATTATAATTCCTATCAACCTGATAAGTTAGGGGAAGGTAGAGCTCTCCTCCAATAAGCCAGATTTCCAGAGTT
TCTGACGTCATAATCTACCAAGGTCATGGATCGAGTTCAGAGAAAAAACAAAAGCAAAACCAAACCTACCAAAAAAT
AAAAATCCCAAAGAAAAAATAAAGAAAAAAACAGCATGAATACTTCCTGCCATGTTAAGTGGCCAATATGTCAGAAA
CAGCACTGAGTTACAGATAAAGATGTCTAAACTACAGTGACATCCCAGCTGTCACAGTGTGTGGACTATTAGTCAAT
AAAACAGTCCCTGCCTCTTAAGAGTTGTTTTCCATGCAAATACATGTCTTATGTCTTAGAATAAGATTCCCTAAGAA
GTGAACCTAGCATTTATACAAGATAATTAATTCTAATCCATAGTATCTGGTAAAGAGCATTCTACCATCATCTTTAC
CGAGCATAGAAGAGCTACACCAAAACCCTGGGTCATCAGCCAGCACATACACTTATCCAGTGATAAATACACATCAT
CGGGTGCCTACATACATACCTGAATATAAAAAAAATACTTTTGCTGAGATGAAACAGGCGTGATTTATTTCAAATAG
GTACGGATAAGTAGATATTGAAGTAAGGATTCAGTCTTATATTATATTACATAACATTAATCTATTCCTGCACTGAA
ACTGTTGCTTTATAGGATTTTTCACTACACTAATGAGAACTTAAGAGATAATGGCCTAAAACCACAGAGAGTATATT
CAAGAATAAGTATAGCACTTCTTATTTGGAAACCAATGCTTACTAAATGAGACTAAGACGTGTCCCATCAAAAATCC
TGGACCTATGCCTAAAACACATTTCACAATCCCTGAACTTTTCAAAAATTGGTACATGCTTTAACTTTAAACTACAG
GCCTCACTGGAGCTACAGACAAGAAGGTGAAAAACGGCTGACAAAAGAAGTCCTGGTATCTTCTATGGTGGGAGAAG
AAAACTAGCTAAAGGGAAGAATAAATTAGAGAAAAATTGGAATGACTGAATCGGAACAAGGCAAAGGCTATAAAAAA
AATTAAGCAGCAGTATCCTCTTGGGGGCCCCTTCCCCACACTATCTCAATGCAAATATCTGTCTGAAACGGTTCCTG
GCTAAACTCCACCCATGGGTTGGCCAGCCTTGCCTTGACCAATAGCCTTGACAAGGCAAACTTGACCAATAGTCTTA
GAGTATCCAGTGAGGCCAGGGGCCGGCGGCTGGCTAGGGATGAAGAATAAAAGGAAGCACCCTTCAGCAGTTCCACA
CACTCGCTTCTGGAACGTCTGAGGTTATCAATAAGCTCCTAGTCCAGACGCCATGGGTCATTTCACAGAGGAGGACA
AGGCTACTATCACAAGCCTGTGGGGCAAGGTGAATGTGGAAGATGCTGGAGGAGAAACCCTGGGAAGGTAGGCTCTG
GTGACCAGGACAAGGGAGGGAAGGAAGGACCCTGTGCCTGGCAAAAGTCCAGGTCGCTTCTCAGGATTTGTGGCACC
TTCTGACTGTCAAACTGTTCTTGTCAATCTCACAGGCTCCTGGTTGTCTACCCATGGACCCAGAGGTTCTTTGACAG
CTTTGGCAACCTGTCCTCTGCCTCTGCCATCATGGGCAACCCCAAAGTCAAGGCACATGGCAAGAAGGTGCTGACTT
CCTTGGGAGATGCCATAAAGCACCTGGATGATCTCAAGGGCACCTTTGCCCAGCTGAGTGAACTGCACTGTGACAAG
CTGCATGTGGATCCTGAGAACTTCAAGGTGAGTCCAGGAGATGTTTCAGCACTGTTGCCTTTAGTCTCGAGGCAACT
TAGACAACTGAGTATTGATCTGAGCACAGCAGGGTGTGAGCTGTTTGAAGATACTGGGGTTGGGAGTGAAGAAACTG
CAGAGGACTAACTGGGCTGAGACCCAGTGGCAATGTTTTAGGGCCTAAGGAGTGCCTCTGAAAATCTAGATGGACAA
CTTTGACTTTGAGAAAAGAGAGGTGGAAATGAGGAAAATGACTTTTCTTTATTAGATTTCGGTAGAAAGAACTTTCA
CCTTTCCCCTATTTTTGTTATTCGTTTTAAAACATCTATCTGGAGGCAGGACAAGTATGGTCGTTAAAAAGATGCAG
GCAGAAGGCATATATTGGCTCAGTCAAAGTGGGGAACTTTGGTGGCCAAACATACATTGCTAAGGCTATTCCTATAT
CAGCTGGACACATATAAAATGCTGCTAATGCTTCATTACAAACTTATATCCTTTAATTCCAGATGGGGGCAAAGTAT
GTCCAGGGGTGAGGAACAATTGAAACATTTGGGCTGGAGTAGATTTTGAAAGTCAGCTCTGTGTGTGTGTGTGTGTG
TGTGCGCGCGTGTGTTTGTGTGTGTGTGAGAGCGTGTGTTTCTTTTAACGTTTTCAGCCTACAGCATACAGGGTTCA
TGGTGGCAAGAAGATAACAAGATTTAAATTATGGCCAGTGACTAGTGCTGCAAGAAGAACAACTACCTGCATTTAAT
GGGAAAGCAAAATCTCAGGCTTTGAGGGAAGTTAACATAGGCTTGATTCTGGGTGGAAGCTTGGTGTGTAGTTATCT
GGAGGCCAGGCTGGAGCTCTCAGCTCACTATGGGTTCATCTTTATTGTCTCCTTTCATCTCAACAGCTCCTGGGAAA
TGTGCTGGTGACCGTTTTGGCAATCCATTTCGGCAAAGAATTCACCCCTGAGGTGCAGGCTTCCTGGCAGAAGATGG
TGACTGGAGTGGCCAGTGCCCTGTCCTCCAGATACCACTGAGCTCACTGCCCATGATGCAGAGCTTTCAAGGATAGG
CTTTATTCTGCAAGCAATACAAATAATAAATCTATTCTGCTAAGAGATCACACATGGTTGTCTTCAGTTCTTTTTTT
TATGTCTTTTTAAATATATGAGCCACAAAGGGTTTTATGTTGAGGGATGTGTTTATGTGTATTTATACATGGCTATG
TGTGTTTGTGTCATGTGCACACTCCACACTTTTTTGTTTACGTTAGATGTGGGTTTTGATGAGCAAATAAAAGAACT
AGGCAATAAAGAAACTTATACATGGGAGCGTCTGCAAGTGGGAGTAAAAGGTGCAGGAGAAATCTGGTTGGAAGAAA
GACCTCTATAGGACAGGACTCCTCAGAAACAGATGTTTTGGAAGAGATGGGGAAAGGTTCAGTGAAGGGGGCTGAAC
CCCCTTCCCTGGATTGCAGCACAGCAGCGAGGAAGGGGCTCAACGAAGAAAAAGTGTTCCAAGCTTTAGGAAGTCAA
GGTTTAGGCAGGGATAGCCATTCTATTTTATTAGGGGCAATACTATTTCCAACGGCATCTGGCTTTTCTCAGCCCTT
GTGAGGCTCTACGGGGAGGTTGAGGTGTTAGAGATCAGAGCAGGAAACAGGTTTTTCTTTCCACGGTAACTACAATG
AAGTGATCCTTACTTTACTAAGGAACTTTTTCATTTTAAGTGTTGACGCATGCCTAAAGAGGTGAAATTAATCCCAT
ACCCTTAAGTCTACAGACTGGTCACAGCATTTCAAGGAGGAGACCTCATTGTAAGCTTCTAGGGAGGTGGGGACCTA
GGTGAAGGAAATGAGCCAGCAGAAGCTCACAAGTCAGCATCAGCGTGTCATGTCTCAGCAGCAGAACAGCACGGTCA
GATGAAAATATAGTGTGAAGAATTTGTATAACATTAATTGAGAAGGCAGATTCACTGGAGTTCTTATATAATTGAAA
GTTAATGCACGTTAATAAGCAAGAGTTTAGTTTAATGTGATGGTGTTATGAACTTAACGCTTGTGTCTCCAGAAAAT
TCACATGCTGAATCCCCAACTCCCAATTGGCTCCATTTGTGGGGGAGGCTTTGGAAAAGTAATCAGGTTTAGAGGAG
CTCATGAGAGCAGATCCCCATCATAGAATTATTTTCCTCATCAGAAGCAGAGAGATTAGCCATTTCTCTTCCTTCTG
GTGAGGACACAGTGGGAAGTCAGCCACCTGCAACCCAGGAAGAGAGCCCTGACCAGGAACCAGCAGAAAAGTGAGAA
AAAATCCTGTTGTTGAAGTCACCCAGTCTATGCTATTTTGTTATAGCACCTTGCACTAAGTAAGGCAGATGAAGAAA
GAGAAAAAAATAAGCTTCGGTGTTCAGTGGATTAGAAACCATGTTTATCTCAGGTTTACAAATCTCCACTTGTCCTC
TGTGTTTCAGAATAAAATACCAACTCTACTACTCTCATCTGTAAGATGCAAATAGTAAGCCTGATCCCTTCTGTCTA
ACTTCGAATTCTATTTTTTCTTCAACGTACTTTAGGCTTGTAATGTGTTTATATACAGTGAAATGTCAAGTTCTTTC
TTTATATTTCTTTCTTTCTTTTTTTTCCTCAGCCTCAGAGTTTTCCACATGCCCTTCCTACCTTCAGGAACTTCTTT
CTCCAAACGTCTTCTGCCTGGCCTCCATTCAAATCATAAAGGACCCACTTCAAATGCCATCACTCACTACCATTTCA
CAATTCGCACTTTCTTTCTTTGTCCTTTTTTTTTTTAGTAAAACAAGTTTATAAAAAATTGAAGGAATAAATGAATG
GCTACTTCATAGGCAGAGTAGACACAAGGGCTACTGGTTGCCGATTTTTATTGTTATTTTTCAATAGTATGCTAAAC
AAGGGGTAGATTATTTATGCTGCCCATTTTTAGACCATAAAAGATAACTTCCTGATGTTGCCATGGCATTTTTTTTC
CTTTTAATTTTATTTCATTTCATTTTAATTTCGAAGGTACATGTGCAGGATGTGCAGGCTTGTTACATGGGTAAATG
TGTGTCTTTCTGGCCTTTTAGCCATCTGTATCAATGAGCAGATATAAGCTTTACACAGGATCATGAAGGATGAAAGA
ATTTCACCAATATTATAATAATTTCAATCAACCTGATAGCTTAGGGGATAAACTAATTTGAAGATACAGCTTGCCTC
CGATAAGCCAGAATTCCAGAGCTTCTGGCATTATAATCTAGCAAGGTTAGAGATCATGGATCACTTTCAGAGAAAAA
CAAAAACAAACTAACCAAAAGCAAAACAGAACCAAAAAACCTCCATAAATACTTCCTACCCAGTTAATGGTCCAATA
TGTCAGAAACAGCACTGTGTTAGAAATAAAGCTGTCTAAAGTACACTAATATTCGAGTTATAATAGTGTGTGGACTA
TTAGTCAATAAAAACAACCCTTGCCTCTTTAGAGTTGTTTTCCATGTACACGCACATCTTATGTCTTAGAGTAAGAT
TCCCTGAGAAGTGAACCTAGCATTTATACAAGATAATTAATTCTAATCCACAGTACCTGCCAAAGAACATTCTACCA
TCATCTTTACTGAGCATAGAAGAGCTACGCCAAAACCCTGGGTCATCAGCCAGCACACACACTTATCCAGTGGTAAA
TACACATCATCTGGTGTATACATACATACCTGAATATGGAATCAAATATTTTTCTAAGATGAAACAGTCATGATTTA
TTTCAAATAGGTACGGATAAGTAGATATTGAGGTAAGCATTAGGTCTTATATTATGTAACACTAATCTATTACTGCG
CTGAAACTGTGGTCTTTATGAAAATTGTTTTCACTACACTATTGAGAAATTAAGAGATAATGGCAAAAGTCACAAAG
AGTATATTCAAAAAGAAGTATAGCACTTTTTCCTTAGAAACCACTGCTAACTGAAAGAGACTAAGATTTGTCCCGTC
AAAAATCCTGGACCTATGCCTAAAACACATTTCACAATCCCTGAACTTTTCAAAAATTGGTACATGCTTTAGCTTTA
AACTACAGGCCTCACTGGAGCTACAGACAAGAAGGTAAAAAACGGCTGACAAAAGAAGTCCTGGTATCCTCTATGAT
GGGAGAAGGAAACTAGCTAAAGGGAAGAATAAATTAGAGAAAAACTGGAATGACTGAATCGGAACAAGGCAAAGGCT
ATAAAAAAAATTAAGCAGCAGTATCCTCTTGGGGGCCCCTTCCCCACACTATCTCAATGCAAATATCTGTCTGAAAC
GGTCCCTGGCTAAACTCCACCCATGGGTTGGCCAGCCTTGCCTTGACCAATAGCCTTGACAAGGCAAACTTGACCAA
TAGTCTTAGAGTATCCAGTGAGGCCAGGGGCCGGCGGCTGGCTAGGGATGAAGAATAAAAGGAAGCACCCTTCAGCA
GTTCCACACACTCGCTTCTGGAACGTCTGAGATTATCAATAAGCTCCTAGTCCAGACGCCATGGGTCATTTCACAGA
GGAGGACAAGGCTACTATCACAAGCCTGTGGGGCAAGGTGAATGTGGAAGATGCTGGAGGAGAAACCCTGGGAAGGT
AGGCTCTGGTGACCAGGACAAGGGAGGGAAGGAAGGACCCTGTGCCTGGCAAAAGTCCAGGTCGCTTCTCAGGATTT
GTGGCACCTTCTGACTGTCAAACTGTTCTTGTCAATCTCACAGGCTCCTGGTTGTCTACCCATGGACCCAGAGGTTC
TTTGACAGCTTTGGCAACCTGTCCTCTGCCTCTGCCATCATGGGCAACCCCAAAGTCAAGGCACATGGCAAGAAGGT
GCTGACTTCCTTGGGAGATGCCATAAAGCACCTGGATGATCTCAAGGGCACCTTTGCCCAGCTGAGTGAACTGCACT
GTGACAAGCTGCATGTGGATCCTGAGAACTTCAAGGTGAGTCCAGGAGATGTTTCAGCACTGTTGCCTTTAGTCTCG
AGGCAACTTAGACAACTGAGTATTGATCTGAGCACAGCAGGGTGTGAGCTGTTTGAAGATACTGGGGTTGGGAGTGA
AGAAACTGCAGAGGACTAACTGGGCTGAGACCCAGTGGCAATGTTTTAGGGCCTAAGGAGTGCCTCTGAAAATCTAG
ATGGACAACTTTGACTTTGAGAAAAGAGAGGTGGAAATGAGGAAAATGACTTTTCTTTATTAGATTTCGGTAGAAAG
AACTTTCACCTTTCCCCTATTTTTGTTATTCGTTTTAAAACATCTATCTGGAGGCAGGACAAGTATGGTCGTTAAAA
AGATGCAGGCAGAAGGCATATATTGGCTCAGTCAAAGTGGGGAACTTTGGTGGCCAAACATACATTGCTAAGGCTAT
TCCTATATCAGCTGGACACATATAAAATGCTGCTAATGCTTCATTACAAACTTATATCCTTTAATTCCAGATGGGGG
CAAAGTATGTCCAGGGGTGAGGAACAATTGAAACATTTGGGCTGGAGTAGATTTTGAAAGTCAGCTCTGTGTGTGTG
TGTGTGTGTGTGTGTGTCAGCGTGTGTTTCTTTTAACGTCTTCAGCCTACAACATACAGGGTTCATGGTGGGAAGAA
GATAGCAAGATTTAAATTATGGCCAGTGACTAGTGCTTGAAGGGGAACAACTACCTGCATTTAATGGGAAGGCAAAA
TCTCAGGCTTTGAGGGAAGTTAACATAGGCTTGATTCTGGGTGGAAGCTGGGTGTGTAGTTATCTGGAGGCCAGGCT
GGAGCTCTCAGCTCACTATGGGTTCATCTTTATTGTCTCCTTTCATCTCAACAGCTCCTGGGAAATGTGCTGGTGAC
CGTTTTGGCAATCCATTTCGGCAAAGAATTCACCCCTGAGGTGCAGGCTTCCTGGCAGAAGATGGTGACTGCAGTGG
CCAGTGCCCTGTCCTCCAGATACCACTGAGCCTCTTGCCCATGATTCAGAGCTTTCAAGGATAGGCTTTATTCTGCA
AGCAATACAAATAATAAATCTATTCTGCTGAGAGATCACACATGATTTTCTTCAGCTCTTTTTTTTACATCTTTTTA
AATATATGAGCCACAAAGGGTTTATATTGAGGGAAGTGTGTATGTGTATTTCTGCATGCCTGTTTGTGTTTGTGGTG
TGTGCATGCTCCTCATTTATTTTTATATGAGATGTGCATTTTGATGAGCAAATAAAAGCAGTAAAGACACTTGTACA
CGGGAGTTCTGCAAGTGGGAGTAAATGGTGTTGGAGAAATCCGGTGGGAAGAAAGACCTCTATAGGACAGGACTTCT
CAGAAACAGATGTTTTGGAAGAGATGGGAAAAGGTTCAGTGAAGACCTGGGGGCTGGATTGATTGCAGCTGAGTAGC
AAGGATGGTTCTTAATGAAGGGAAAGTGTTCCAAGCTTTAGGAATTCAAGGTTTAGTCAGGTGTAGCAATTCTATTT
TATTAGGAGGAATACTATTTCTAATGGCACTTAGCTTTTCACAGCCCTTGTGGATGCCTAAGAAAGTGAAATTAATC
CCATGCCCTCAAGTGTGCAGATTGGTCACAGCATTTCAAGGGAGAGACCTCATTGTAAGACTCTGGGGGAGGTGGGG
ACTTAGGTGTAAGAAATGAATCAGCAGAGGCTCACAAGTCAGCATGAGCATGTTATGTCTGAGAAACAGACCAGCAC
TGTGAGATCAAAATGTAGTGGGAAGAATTTGTACAACATTAATTGGAAGGTTTACTTAATGGAATTTTTGTATAGTT
GGATGTTAGTGCATCTCTATAAGTAAGAGTTTAATATGATGGTGTTACGGACCTGGTGTTTGTGTCTCCTCAAAATT
CACATGCTGAATCCCCAACTCCCAACTGACCTTATCTGTGGGGGAGGCTTTTGAAAAGTAATTAGGTTTAGCTGAGC
TCATAAGAGCAGATCCCCATCATAAAATTATTTTCCTTATCAGAAGCAGAGAGACAAGCCATTTCTCTTTCCTCCCG
GTGAGGACACAGTGAGAAGTCCGCCATCTGCAATCCAGGAAGAGAACCCTGACCACGAGTCAGCCTTCAGAAATGTG
AGAAAAAACTCTGTTGTTGAAGCCACCCAGTCTTTTGTATTTTGTTATAGCACCTTACACTGAGTAAGGCAGATGAA
GAAGGAGAAAAAAATAAGCTTGGGTTTTGAGTGAACTACAGACCATGTTATCTCAGGTTTGCAAAGCTCCCCTCGTC
CCCTATGTTTCAGCATAAAATACCTACTCTACTACTCTCATCTATAAGACCCAAATAATAAGCCTGCGCCCTTCTCT
CTAACTTTGATTTCTCCTATTTTTACTTCAACATGCTTTACTCTAGCCTTGTAATGTCTTTACATACAGTGAAATGT
AAAGTTCTTTATTCTTTTTTTCTTTCTTTCTTTTTTCTCCTCAGCCTCAGAATTTGGCACATGCCCTTCCTTCTTTC
AGGAACTTCTCCAACATCTCTGCCTGGCTCCATCATATCATAAAGGTCCCACTTCAAATGCAGTCACTACCGTTTCA
GGATATGCACTTTCTTTCTTTTTTGTTTTTTGTTTTTTTTAAGTCAAAGCAAATTTCTTGAGAGAGTAAAGAAATAA
ACGAATGACTACTGCATAGGCAGAGCAGCCCCGAGGGCCGCTGGTTGTTCCTTTTATGGTTATTTCTTGATGATATG
TTAAACAAGTTTTGGATTATTTATGCCTTCTCTTTTTAGGCCATATAGGGTAACTTTCTGACATTGCCATGGCATGT
TTCTTTTAATTTAATTTACTGTTACCTTAAATTCAGGGGTACACGTACAGGATATGCAGGTTTGTTTTATAGGTAAA
AGTGTGCCATGGTTTTAATGGGTTTTTTTTTTCTTGTAAAGTTGTTTAAGTTTCTTGTTTACTCTGGATATTGGCCT
TTGTCAGAAGAATAGATTGGAAAATCTTTTTCCCATTCTGTAGATTGTCTTTCGCTCTGATGGTAGTTTCTTTTGCT
GAGCAGGAGCTCTTTAGTTTAATTAGATTCCATTGGTCAATTTTTGCTTTTGCTGCAATTGCTTTTCACGCTTTCAT
CATGAAATCTGTGCCCGTGTTTATATCATGAATAGTATTGCCTTGATTTTTTTCTAGGCTTTTTATAGTTTGGGGTT
TTTCATTTAAGTCTCTAATCCATCCGGAGTTAATTTTGGATAAGGTATAAGGAAGGAGTCCAGTTTCATTTTTCAGC
ATATGGCTAGCCAGTTCTCCCCCATCATTTATTAAATTGAAAATCCTTTCCCCATTGCTTGCTTTTGTCAGGTTTCT
AAAAGACAGATGGTTGTAGGTACAATATGCAGTTTCTTCAAGTCATATAATACCATCTGAAATCTCTTATTAATTCA
TTTCTTTTAGTATGTATGCTGGTCTCCTCTGCTCACTATAGTGAGGGCACCATTAGCCAGAGAATCTGTCTGTCTAG
TTCATGTAAGATTCTCAGAATTAAGAAAAATGGATGGCATATGAATGAAACTTCATGGATGACATATGGAATCTAAT
GTGTATTTGTTGAATTAATGCATAAGATGCAACAAGGGAAAGGTTGACAACTGCAGTGATAACCTGGTATTGATGAT
ATAAGAGTCTATAGATCACAGTAGAAGCAATAATCATGGAAAACAATTGGAAATGGGGAACAGCCACAAACAAGAAA
GAATCAATACTACCAGGAAAGTGACTGCAGGTCACTTTTCCTGGAGCGGGTGAGAGAAAAGTGGAAGTTGCAGTAAC
TGCCGAATTCCTGGTTGGCTGATGGAAAGATGGGGCAACTGTTCACTGGTACGCAGGGTTTTAGATGTATGTACCTA
AGGATATGAGGTATGGCAATGAACAGAAATTCTTTTGGGAATGAGTTTTAGGGCCATTAAAGGACATGACCTGAAGT
TTCCTCTGAGGCCAGTCCCCACAACTCAATATAAATGTGTTTCCTGCATATAGTCAAAGTTGCCACTTCTTTTTCTT
CATATCATCGATCTCTGCTCTTAAAGATAATCTTGGTTTTGCCTCAAACTGTTTGTCACTACAAACTTTCCCCATGT
TCCTAAGTAAAACAGGTAACTGCCTCTCAACTATATCAAGTAGACTAAAATATTGTGTCTCTAATATCAGAAATTCA
GCTTTAATATATTGGGTTTAACTCTTTGAAATTTAGAGTCTCCTTGAAATACACATGGGGGTGATTTCCTAAACTTT
ATTTCTTGTAAGGATTTATCTCAGGGGTAACACACAAACCAGCATCCTGAACCTCTAAGTATGAGGACAGTAAGCCT
TAAGAATATAAAATAAACTGTTCTTCTCTCTGCCGGTGGAAGTGTGCCCTGTCTATTCCTGAAATTGCTTGTTTGAG
ACGCATGAGACGTGCAGCACATGAGACACGTGCAGCAGCCTGTGGAATATTGTCAGTGAAGAATGTCTTTGCCTGAT
TAGATATAAAGACAAGTTAAACACAGCATTAGACTATAGATCAAGCCTGTGCCAGACACAAATGACCTAATGCCCAG
CACGGGCCACGGAATCTCCTATCCTCTTGCTTGAACAGAGCAGCACACTTCTCCCCCAACACTATTAGATGTTCTGG
CATAATTTTGTAGATATGTAGGATTTGACATGGACTATTGTTCAATGATTCAGAGGAAATCTCCTTTGTTCAGATAA
GTACACTGACTACTAAATGGATTAAAAAACACAGTAATAAAACCCAGTTTTCCCCTTACTTCCCTAGTTTGTTTCTT
ATTCTGCTTTCTTCCAAGTTGATGCTGGATAGAGGTGTTTATTTCTATTCTAAAAAGTGATGAAATTGGCCGGGCGC
GGTGGCTCACACCTGTAATCCCAGCACTTTGGGAGGCTGAGGTGGGCGGATCACGAGGTCAGGAGATCAAGACCATC
CTGGCTAACATGGTGAAACCCCATCTCTACTAAAAATACAAAAAATTAGCCAGAGACGGTGGCGGGTGCCTGTAGTC
CCAGCTACTCGGGAGGCTGAGGCAGGAGAATGGCGTGAACCTGGGAGGCAGAGCTGCAGTGAGCAGAGATCGCGCCA
CTGCACACTCCAGCCTGGGTGACAAAGCGAGACTCCATCTCAAAAAAAAAAAAAAAAAAAAAAAGAAAGAAAGAAAG
AAAAAAAAAGTGATGAAATTGTGTATTCAATGTAGTCTCAAGAGAATTGAAAACCAAGAAAGGCTGTGGCTTCTTCC
ACATAAAGCCTGGATGAATAACAGGATAACACGTTGTTACATTGTCACAACTCCTGATCCAGGAATTGATGGCTAAG
ATATTCGTAATTCTTATCCTTTTCAGTTGTAACTTATTCCTATTTGTCAGCATTCAGGTTATTAGCGGCTGCTGGCG
AAGTCCTTGAGAAATAAACTGCACACTGGATGGTGGGGGTAGTGTAGGAAAATGGAGGGGAAGGAAGTAAAGTTTCA
AATTAAGCCTGAACAGCAAAGTTCCCCTGAGAAGGCCACCTGGATTCTATCAGAAACTCGAATGTCCATCTTGCAAA
ACTTCCTTGCCCAAACCCCACCCCTGGAGTCACAACCCACCCTTGACCAATAGATTCATTTCACTGAGGGAGGCAAA
GGGCTGGTCAATAGATTCATTTCACTGGGAGAGGCAAAGGGCTGGGGGCCAGAGAGGAGAAGTAAAAAGCCACACAT
GAAGCAGCAATGCAGGCATGCTTCTGGCTCATCTGTGATCACCAGGAAACTCCCAGATCTGACACTGTAGTGCATTT
CACTGCTGACAAGAAGGCTGCTGCCACCAGCCTGTGAAGCAAGGTTAAGGTGAGAAGGCTGGAGGTGAGATTCTGGG
CAGGTAGGTACTGGAAGCCGGGACAAGGTGCAGAAAGGCAGAAAGTGTTTCTGAAAGAGGGATTAGCCCGTTGTCTT
ACATAGTCTGACTTTGCACCTGCTCTGTGATTATGACTATCCCACAGTCTCCTGGTTGTCTACCCATGGACCTAGAG
GTACTTTGAAAGTTTTGGATATCTGGGCTCTGACTGTGCAATAATGGGCAACCCCAAAGTCAAGGCACATGGCAAGA
AGGTGCTGATCTCCTTCGGAAAAGCTGTTATGCTCACGGATGACCTCAAAGGCACCTTTGCTACACTGAGTGACCTG
CACTGTAACAAGCTGCACGTGGACCCTGAGAACTTCCTGGTGAGTAGTAAGTACACTCACGCTTTCTTCTTTACCCT
TAGATATTTGCACTATGGGTACTTTTGAAAGCAGAGGTGGCTTTCTCTTGTGTTATGAGTCAGCTATGGGATATGAT
ATTTCAGCAGTGGGATTTTGAGAGTTATGTTGCTGTAAATAACATAACTAAAATTTGGTAGAGCAAGGACTATGAAT
AATGGAAGGCCACTTACCATTTGATAGCTCTGAAAAACACATCTTATAAAAAATTCTGGCCAAAATCAAACTGAGTG
TTTTGGATGAGGGAACAGAAGTTGAGATAGAGAAAATAACATCTTTCCTTTGGTCAGCGAAATTTTCTATAAAAATT
AATAGTCACTTTTCTGCATAGTCCTGGAGGTTAGAAAAAGATCAACTGAACAAAGTAGTGGGAAGCTGTTAAAAGAG
GATTGTTTCCCTCCGAATGATGATGGTATACTTTTGTACGCATGGTACAGGATTCTTTGTTATGAGTGTTTGGGAAA
ATTGTATGTATGTATGTATGTATGTGATGACTGGGGACTTATCCTATCCATTACTGTTCCTTGAAGTACTATTATCC
TACTTTTTAAAAGGACGAAGTCTCTAAAAAAAAAATGAAACAATCACAATATGTTGGGGTAGTGAGTTGGCATAGCA
AGTAAGAGAAGGATAGGACACAATGGGAGGTGCAGGGCTGCCAGTCATATTGAAGCTGATATCTAGCCCATAATGGT
GAGAGTTGCTCAAACTCTGGTCAAAAAGGATGTAAGTGTTATATCTATTTACTGCAAGTCCAGCTTGAGGCCTTCTA
TTCACTATGTACCATTTTCTTTTTTATCTTCACTCCCTCCCCAGCTCTTAGGCAACGTGATATTGATTGTTTTGGCA
ACCCACTTCAGCGAGGATTTTACCCTACAGATACAGGCTTCTTGGCAGTAACTAACAAATGCTGTGGTTAATGCTGT
AGCCCACAAGACCACTGAGTTCCCTGTCCACTATGTTTGTACCTATGTCCCAAAATCTCATCTCCTTTAGATGGGGG
AGGTTGGGGAGAAGAGCAGTATCCTGCCTGCTGATTCAGTTCCTGCATGATAAAAATAGAATAAAGAAATATGCTCT
CTAAGAAATATCATTGTACTCTTTTTCTGTCTTTATATTTTACCCTGATTCAGCCAAAAGGACGCACTATTTCTGAT
GGAAATGAGAATGTTGGAGAATGGGAGTTTAAGGACAGAGAAGATACTTTCTTGCAATCCTGCAAGAAAAGAGAGAA
CTCGTGGGTGGATTTAGTGGGGTAGTTACTCCTAGGAAGGGGAAATCGTCTCTAGAATAAGACAATGTTTTTACAGA
AAGGGAGGTCAATGGAGGTACTCTTTGGAGGTGTAAGAGGATTGTTGGTAGTGTGTAGAGGTATGTTAGGACTCAAA
TTAGAAGTTCTGTATAGGCTATTATTTGTATGAAACTCAGGATATAGCTCATTTGGTGACTGCAGTTCACTTCTACT
TATTTTAAACAACATATTTTTTATGATTTATAATGAAGTGGGGATGGGGCTTCCTAGAGACCAATCAAGGGCCAAAC
CTTGAACTTTCTCTTAACGTCTTCAATGGTATTAATAGAGAATTATCTCTAAGGCATGTGAACTGGCTGTCTTGGTT
TTCATCTGTACTTCATCTGCTACCTCTGTGACCTGAAACATATTTATAATTCCATTAAGCTGTGCATATGATAGATT
TATCATATGTATTTTCCTTAAAGGATTTTTGTAAGAACTAATTGAATTGATACCTGTAAAGTCTTTATCACACTACC
CAATAAATAATAAATCTCTTTGTTCAGCTCTCTGTTTCTATAAATATGTACAAGTTTTATTGTTTTTAGTGGTAGTG
ATTTTATTCTCTTTCTATATATATACACACACATGTGTGCATTCATAAATATATACAATTTTTATGAATAAAAAATT
ATTAGCAATCAATATTGAAAACCACTGATTTTTGTTTATGTGAGCAAACAGCAGATTAAAAGGCTGAGATTTAGGAA
ACAGCACGTTAAGTCAAGTTGATAGAGGAGAATATGGACATTTAAAAGAGGCAGGATGATATAAAATTAGGGAAACT
GGATGCAGAGACCAGATGAAGTAAGAAAAATAGCTATCGTTTTGAGCAAAAATCACTGAAGTTTCTTGCATATGAGA
GTGACATAATAAATAGGGAAACGTAGAAAATTGATTCACATGTATATATATATATAGAACTGATTAGACAAAGTCTA
ACTTGGGTATAGTCAGAGGAGCTTGCTGTAATTATATTGAGGTGATGGATAAAGAACTGAAGTTGATGGAAACAATG
AAGTTAAGAAAAAAAATCGAGTAAGAGACCATTGTGGCAGTGATTGCACAGAACTGGAAAACATTGTGAAACAGAGA
GTCAGAGATGACAGCTAAAATCCCTGTCTGTGAATGAAAAGAAGGAAATTTATTGACAGAACAGCAAATGCCTACAA
GCCCCCTGTTTGGATCTGGCAATGAACGTAGCCATTCTGTGGCAATCACTTCAAACTCCTGTACCCAAGACCCTTAG
GAAGTATGTAGCACCCTCAAACCTAAAACCTCAAAGAAAGAGGTTTTAGAAGATATAATACCCTTTCTTCTCCAGTT
TCATTAATCCCAAAACCTCTTTCTCAAAGTATTTCCTCTATGTGTCCACCCCAAAGAGCTCACCTCACCATATCTCT
TGAGTGGGAGCACATAGATAGGCGGTGCTACCATCTAACAGCTTCTGAAATTCCTTTGTCATATTTTTGAGTCCCCA
CTAATAACCCACAAAGCAGAATAAATACCAGTTGCTCATGTACAATAATCACTCAACTGCTGTCTTGTAGCATACAT
TAATTAAGCACATTCTTTGAATAATTACTGTGTCCAAACAATCACACTTTAAAATCTCACACTTGTGCTATCCCTTG
CCCTTCTGAATGTCACTCTGTATTTTAAATGAAGAGATGAGGGTTGAATTTCCTGTGTTACTTATTGTTCATTTCTC
GATGAGGAGTTTTCACATTCACCTTTACTGGAAAACACATAAGTACACATCTTACAGGAAAAATATACCAAACTGAC
ATGTAGCATGAATGCTTGTGCATGTAGTCATATAAAATCTTGTAGCAATGTAAACATTCTCTGATATACACATACAG
ATGTGTCTATATGTCTACACAATTTCTTATGCTCCATGAACAAACATTCCATGCACACATAAGAACACACACTGTTA
CAGATGCATACTTGAGTGCATTGACAAAATTACCCCAGTCAATCTAGAGAATTTGGATTTCTGCATTTGACTCTGTT
AGCTTTGTACATGCTGTTCATTTACTCTGGGTGATGTCTTTCCCTCATTTTGCCTTGTCTATCTTGTACTCATACTT
TAAGTCCTAACTTATATGTTATCTCAACTAAGAAGCTATTTTTTTTTAATTTTAACTGGGCTTAAAGCCCTGTCTAT
AAACTCTGCTACAATTATGGGCTCTTTCTTATAATATTTAGTGTTTTTCCTACTAATGTACTTAATCTGCTCATTGT
ATATTCCTACCACTAAATTTTAACCTCTTTTATGGTAGAGACATTGTCTTGTAAACTCTTATTTCCCTAGTATTTGG
AGATGAAAAAAAAGATTAAATTATCCAAAATTAGATCTCTCTTTTCTACATTATGAGTATTACACTATCCATAGGGA
AGTTTGTTTGAGACCTAAACTGAGGAACCTTTGGTTCTAAAATGACTATGTGATATCTTAGTATTTATAGGTCATGA
GGTTCCTTCCTCTGCCTCTGCTATAGTTTGATTAGTCAGCAAGCATGTGTCATGCATTTATTCACATCAGAATTTCA
TACACTAATAAGACATAGTATCAGAAGTCAGTTTATTAGTTATATCAGTTAGGGTCCATCAAGGAAAGGACAAACCA
TTATCAGTTACTCAACCTAGAATTAAATACAGCTCTTAATAGTTAATTATCCTTGTATTGGAAGAGCTAAAATATCA
AATAAAGGACAGTGCAGAAATCTAGATGTTAGTAACATCAGAAAACCTCTTCCGCCATTAGGCCTAGAAGGGCAGAA
GGAGAAAATGTTTATACCACCAGAGTCCAGAACCAGAGCCCATAACCAGAGGTCCACTGGATTCAGTGAGCTAGTGG
GTGCTCCTTGGAGAGAGCCAGAACTGTCTAATGGGGGCATCAAAGTATCAGCCATAAAAAACCATAAAAAAGACTGT
CTGCTGTAGGAGATCCGTTCAGAGAGAGAGAGAGACCAGAAATAATCTTGCTTATGCTTTCCCTCAGCCAGTGTTTA
CCATTGCAGAATGTACATGCGACTGAAAGGGTGAGGAAACCTGGGAAATGTCAGTTCCTCAAATACAGAGAACACTG
AGGGAAGGATGAGAAATAAATGTGAAAGCAGACATGAATGGTAATTGACAGAAGGAAACTAGGATGTGTCCAGTAAA
TGAATAATTACAGTGTGCAGTGATTATTGCAATGATTAATGTATTGATAAGATAATATGAAAACACAGAATTCAAAC
AGCAGTGAACTGAGATTAGAATTGTGGAGAGCACTGGCATTTAAGAATGTCACACTTAGAATGTGTCTCTAGGCATT
GTTCTGTGCATATATCATCTCAATATTCATTATCTGAAAATTATGAATTAGGTACAAAGCTCAAATAATTTATTTTT
TCAGGTTAGCAAGAACTTTTTTTTTTTTTTTTTCTGAGATGGAGCATTGCTATGGTTGCCCAGGCTGGAGTGCAATG
GCATGATCCAGGCTCACTGCAACATCTGCCTCCCAGGTTCAAGCGATTCTCCTGCCTCAGCCTCCCAAGTAGCTGGC
ATTACAGGCATGTGCCACCACCATGCCTGGCTAATTTTCTATTTTTAGTAGATAGGGGGTTTCACCATGTTGGTCAG
GCTGATCTCGAACTCCTAACATCAGGTGATCCACCCTCCTCGGCCTCTGAATGTACTGGGATCACAGGCGTGAGCCA
CCACACCCAGCCAAGAATGTGAATTTTGTAGAAGGATATAACCCATATTTCTCTGACCCTAGAGTCCTTAGTATACC
TCCCATACCATGTGGCTCATCCTCCTTACATACATTTCCCATCTTTCACCCTACCTTTTCCTTTTTGTTTCAGCTTT
TCACTGTGTGTCAAAATCTAGAACCTTATCTCCTACCTGCTCTGAAACCAACAGCAAGTTGACTTCCATTCTAACCC
ACATTGGCATTACACTAATTAAAATCGATACTGAGTTCTAAAATCATCTGGGATTTTGGGGACTATGTCTTACTTCA
TACTTCCTTGAGATTTCACATTAAATGTTGGTGTTCATTAAAGGTCCTTCATTTAACTTTGTATTCATCACACTCTT
GGATTCACAGTTATATCTAAACTCTTATATATAGCCTGTATAATCCCAATTCCCAAGTCTGATTTCTAACCTCTGAC
CTCCAACCTCAGTGCCAAACCCATATATCAAACAATGTACTGGGCTTATTTATATAGATGTCCTATAGGCACCTCAG
ACTCAGCATGGGTATTTCACTTGTTATACTAAAACTGTTTCTCTTCCAGTGTTTTCCATTTTAGTCATTAGATAGCT
ACTTGCCCATTCACCAAGGTCACAGATTAAAATCATTTCCCTACCTCTAATCAACAGTTCAATTCTGCTTCAATTTG
TCCCTATCTATTAATCACCACTCTTACTGCCCAGTCAGGTCCTCATTGTTTCCTGAACAAGAGTAGATGCTATTCTT
TCCACTTTAAGACCTTATCCTGGCTGGATGCGGTGGCTCAGGCTTGTAAACCCAGCACTTTGGGAGGCCGAGGCAGG
CAGATCACTTGAGGTCAGGAGTTCAAGACCAGCCTGACCAACATGGTGAAACCCCATCTCTACTAAAAATACAAAAT
CAGCCGGGCGTGTGGTGCATGCCTGCAGTCCCAGCTATTCAGGTGGCTGAGGCAGGAGAATTGCTTGAACCCAGGAG
GCGGAGGTTGCGGTGAGCCTAGATTGCACCATTGCACTCTAGCTTGGGCAATAGGGATGAAACTCCATCTCAGAAGA
GAAAAGAAAAAAAGACCTTATTCTGTTACACAAATCCTCTCAATGCAATCCATATAGAATAAACATGTAACCAGATC
TCCCAATGTGTAAAATCATTTCAGGTAGAACAGAATTAAAGTGAAAAGCCAAGTCTTTGGAATTAACAGACAAAGTT
CAAATAACAGTCCTCATGGCCTTAAGAATTTACCTAACATTTTTTTTAGAATCAATTTTCTTATATATGAATTGGAA
ACATAATTCCTCCCTCACAAACACATTCTAAGATTTTAAGGAGATATTGATGAAGTACATCATCTGTCATTTTTAAC
AGTTAGTGGTAGTGATTCACACAGCACATTATGATCTGTTCTTGTATGTTCTGTTCCATTCTGTATTCTTGACCTGG
TTGTATTCTTTCTGAGCTCCAGATCCACATATCTAAGTACATCTTTTTGCATTTTACAAGAGTGCATACAATACAAT
GTATCCAAGACTGTATTTCTGATTTTATCGTACCACTAAACTCACAAATGTGGCCCTATTCTTGTGTTCACGACTGA
CATCACCGTCATGGTCCAAGTCTGATAATAGAAATGGCATTGTCACTTTCTTCCCTACTGCAACAGAAGCCCAGCTA
TTTGTCTCCCATTTTCTCTACTTCTAAAATACATTTCTTCACTAAGTGAGAATAATCTTTTAAAGACACAAATCAAA
CCATGCCACCACCTTTCTTGAATTATTCAATATCTTTCGTTGGCTTCCAGGTTACAGAAAAATAACTTGTAACAAAG
TTTAAAGGTCATTCATGGCTCCTCTCTACCCTATTTTATAACATTTCCCCTTGTGATCAGAATCTCAGGCACATCAT
CCATCTTTCTATATACAAATAAAGTCATATAGTTTGAACTCACCTCTGGTTACTTTTAATCAACCAAATGCTGTAAA
ATGCATTTGTATCGCTACGTGTTAAGCAGTAGTTGATTCTTTTCATTTCTTGTTAATATTCTATTCTTTGACTATAC
CGTAATTTATCAATTCTACTGTTGGTAAGCATTTAAGTGGCTACCGGTTTGAGGTTTTTATGATTATTGCTGTCATA
AGCATTTCTATACATGTCTTTGGATACACACATGCATGTGTTTCTGAATATCTAAAAATGTAATTGCTAGGTAATAG
ACTTATCAAGCATCCAGCATTTGTGGATACTATTAAAGGTTTTCCAAAGGGGTTATACTATTGTACAGTGTCACCAA
CAGAGTTTGAGTTTCTATTGATCCATATCACCACCAAAATTTGAACTGTCAGTCTTATCTCTTCTCTTGTCTCTTTT
TTCCTCTTTTTTTTCCTTCCCTTCCCCTCTCTTCGTTTCTTTTCTCTCCTCTTCTCTTCTTTCCTCTCTTCCCTTCC
CTTTCTCTTTCTCTTCCCTATCCCTTCTCCTCTCCTCTCCCCTCCTTTTTTCTCCTCTCCTCTCCATTATTTATTTT
TCCTTCTTCTCCTCCATCCCTTCCATCCTCTCTCTTCCCCTCTTCCTTCCTTCCTTTCTCCATTTCTTCCTCCTCTT
TCCCTCAATCCTTCCTTTTGGATATGCTCATGGGTGTGTATTTGTCTGCCATTGTGGCATTATTTGAATTCAGAAAA
GAGTGAAAAACTACTGGGATCTTCATTCTGGGTCTAATTCCACATTTTTTTTTAAGAACACACTCTGTAAAAATGTT
CTGTACTAGCATATTCCCAGGAACTTCGTTAAATTTAATCTGGCTGAATATGGTAAATCTACTTTGCACTTTGCATT
CTTTCTTTAGTCATACCATAATTTTAAACATTCAAAATATTTGTATATAATATTTGATTTTATCTGTCATTAAAATG
TTAACCTTAAAATTCATGTTTCCAGAACCTATTTCAATAACTGGTAAATAAACACTATTCATTTTTTAAATATTCTT
TTAATGGATATTTATTTCAATATAATAAAAAATTAGAGTTTTATTATAGGAAGAATTTACCAAAAGAAGGAGGAAGC
AAGCAAGTTTAAACTGCAGCAATAGTTGTCCATTCCAACCTCTCAAAATTCCCTTGGAGACAAAATCTCTAGAGGCA
AAGAAGAACTTTATATTGAGTCAACTTGTTAAAACATCTGCTTTTAGATAAGTTTTCTTAGTATAAAGTGACAGAAA
CAAATAAGTTAAACTCTAAGATACATTCCACTATATTAGCCTAAAACACTTCTGCAAAAATGAAACTAGGAGGATAT
TTTTAGAAACAACTGCTGAAAGAGATGCGGTGGGGAGATATGCAGAGGAGAACAGGGTTTCTGAGTCAAGACACACA
TGACAGAACAGCCAATCTCAGGGCAAGTTAAGGGAATAGTGGAATGAAGGTTCATTTTTCATTCTCACAAACTAATG
AAACCCTGCTTATCTTAAACCAACCTGCTCACTGGAGCAGGGAGGACAGGACCAGCATAAAAGGCAGGGCAGAGTCG
ACTGTTGCTTACACTTTCTTCTGACATAACAGTGTTCACTAGCAACCTCAAACAGACACCATGGTGCATCTGACTCC
TGAGGAGAAGACTGCTGTCAATGCCCTGTGGGGCAAAGTGAACGTGGATGCAGTTGGTGGTGAGGCCCTGGGCAGGT
TGGTATCAAGGTTATAAGAGAGGCTCAAGGAGGCAAATGGAAACTGGGCATGTGTAGACAGAGAAGACTCTTGGGTT
TCTGATAGGCACTGACTCTCTGTCCCTTGGGCTGTTTTCCTACCCTCAGATTACTGGTGGTCTACCCTTGGACCCAG
AGGTTCTTTGAGTCCTTTGGGGATCTGTCCTCTCCTGATGCTGTTATGGGCAACCCTAAGGTGAAGGCTCATGGCAA
GAAGGTGCTAGGTGCCTTTAGTGATGGCCTGGCTCACCTGGACAACCTCAAGGGCACTTTTTCTCAGCTGAGTGAGC
TGCACTGTGACAAGCTGCACGTGGATCCTGAGAACTTCAGGGTGAGTCCAGGAGATGCTTCACTTTTCTCTTTTTAC
TTTCTAATCTTACATTTTGGTTCTTTTACCTACCTGCTCTTCTCCCACATTTTTGTCATTTTACTATATTTTATCAT
TTAATGCTTCTAAAATTTTGTTAATTTTTTATTTAAATATTCTGCATTTTTTCCTTCCTCACAATCTTGCTATTTTA
AATTATTTAATATCCTGTCTTTCTCTCCCAACCCCCTCCCTTCATTTTTCCTTCTCTAACAACAACTCAAATTATGC
ATACCAGCTCTCACCTGCTAATTCTGCACTTAGAATAATCCTTTTGTCTCTCCACATGGGTATGGGAGAGGCTCCAA
CTCAAAGATGAGAGGCATAGAATACTGTTTTAGAGGCTATAAATCATTTTACAATAAGGAATAATTGGAATTTTATA
AATTCTGTAGTAAATGGAATGGAAAGGAAAGTGAATATTTGATTATGAAAGACTAGGCAGTTACACTGGAGGTGGGG
CAGAAGTCGTTGCTAGGAGACAGCCCATCATCACACTGATTAATCAATTAATTTGTATCTATTAATCTGTTTATAGT
AATTAATTTGTATATGCTATATACACATACAAAATTAAAACTAATTTGGAATTAATTTGTATATAGTATTATACAGC
ATATATAGCATATATGTACATATATAGACTACATGCTAGTTAAGTACATAGAGGATGTGTGTGTATAGATATATGTT
ATATGTATGCATTCATATATGTACTTATTTATGCTGATGGGAATAACCTGGGGATCAGTTTTGTCTAAGATTTGGGC
AGAAAAAAATGGGTGTTGGCTCAGTTTCTCAGAAGCCAGTCTTTATTTCTCTGTTAACCATATGCATGTATCTGCCT
ACCTCTTCTCCGCAGCTCTTGGGCAATGTGCTGGTGTGTGTGCTGGCCCGCAACTTTGGCAAGGAATTCACCCCACA
AATGCAGGCTGCCTATCAGAAGGTGGTGGCTGGTGTGGCTAATGCCCTGGCTCACAAGTACCATTGAGATCCTGGAC
TGTTTCCTGATAACCATAAGAAGACCCTATTTCCCTAGATTCTATTTTCTGAACTTGGGAACACAATGCCTACTTCA
AGGGTATGGCTTCTGCCTAATAAAGAATGTTCAGCTCAACTTCCTGATTAATTTCACTTATTTCATTTTTTTGTCCA
GGTGTGTAAGAAGGTTCCTGAGGCTCTACAGATAGGGAGCACTTGTTTATTTTACAAAGAGTACATGGGAAAAGAGA
AAAGCAAGGGAACCGTACAAGGCATTAATGGGTGACACTTCTACCTCCAAAGAGCAGAAATTATCAAGAACTCTTGA
TACAAAGATAATACTGGCACTGCAGAGGTTCTAGGGAAGACCTCAACCCTAAGACATAGCCTCAAGGGTAATAGCTA
CGATTAAACTCCAACAATTACTGAGAAAATAATGTGCTCAATTAAAGGCATAATGATTACTCAAGACAATGTTATGT
TGTCTTTCTTCCTCCTTCCTTTGCCTGCACATTGTAGCCCATAATACTATACCCCATCAAGTGTTCCTGCTCCAAGA
AATAGCTTCCTCCTCTTACTTGCCCCAGAACATCTCTGTAAAGAATTTCCTCTTATCTTCCCATATTTCAGTCAAGA
TTCATTGCTCACGTATTACTTGTGACCTCTCTTGACCCCAGCCACAATAAACTTCTCTATACTACCCAAAAAATCTT
TCCAAACCCTCCCCGACACCATATTTTTATATTTTTCTTATTTATTTCATGCACACACACACACTCCGTGCTTTATA
AGCAATTCTGCCTATTCTCTACCTTCTTACAATGCCTACTGTGCCTCATATTAAATTCATCAATGGGCAGAAAGAAA
ATATTTATTCAAGAAAACAGTGAATGAATGAACGAATGAGTAAATGAGTAAATGAAGGAATGATTATTCCTTGCTTT
AGAACTTCTGGAATTAGAGGACAATATTAATAATACCATCGCACAGTGTTTCTTTGTTGTTAATGCTACAACATACA
AAGAGGAAGCATGCAGTAAACAACCGAACAGTTATTTCCTTTCTGATCATAGGAGTAATATTTTTTTCCTTGAGCAC
ATTTTTGCCATAGGTAAAATTAGAAGGATTTTTAGAACTTTCTCAGTTGTATACATTTTTAAAAATCTGTATTATAT
GCATGTTGATTAATTTTAAACTTACTTGAATACCTAAACAGAATCTGTTGTTTCCTTGTGTTTGAAAGTGCTTTCAC
AGTAACTCTGTCTGTACTGCCAGAATATACTGACAATGTGTTATAGTTAACTGTTTTGATCACAACATTTTGAATTG
ACTGGCAGCAGAAGCTCTTTTTATATCCATGTGTTTTCCTTAAGTCATTATACATAGTAGGCATGAGACTCTTTATA
CTGAATAAGATATTTAGGAACCACTGGTTTACATATCAGAAGCAGAGCTACTCAGGGCATTTTGGGGAAGATCACTT
TCACATTCCTGAGCATAGGGAAGTTCTCATAAGAGTAAGATATTAAAAGGAGATACTTGTGTGGTATTCGAAAGACA
GTAAGAGAGATTGTAGACCTTATGATCTTGATAGGGAAAACAAACTACATTCCTTTCTCCAAAAGTCAAAAAAAAAG
AGCAAATATAGCTTACTATACCTTCTATTCCTACACCATTAGAAGTAGTCAGTGAGTCTAGGCAAGATGTTGGCCCT
AAAAATCCAAATACCAGAGAATTCATGAGAACATCACCTGGATGGGACATGTGCCGAGCAACACAATTACTATATGC
TAGGCATTGCTATCTTCATATTGAAGATGAGGAGGTCAAGAGATGAAAAAAGACTTGGCACCTTGTTGTTATATTAA
AATTATTTGTTAGAGTAGAGCTTTTGTAAGAGTCTAGGAGTGTGGGAGCTAAATGATGATACACATGGACACAAAGA
ATAGATCAACAGACACCCAGGCCTACTTGAGGGTTGAGGGTGGGAAGAGGGAGACGATGAAAAAGAACCTATTGGGT
ATTAAGTTCATCACTGAGTGATGAAATAATCTGTACATCAAGACCCAGTGATATGCAATTTACCTATATAACTTGTA
CATGTACCCCCAAATTTAAAATAAAGTTAAAACAAAGTATAGGAATGGAATTAATTCCTCAAGATTTGGCTTTAATT
TTATTTGATAATTTATCAAATGGTTGTTTTTCTTTTCTCACTATGGCGTTGCTTTATAAACTATGTTCAGTATGTCT
GAATGAAAGGGTGTGTGTGTGTGTGAAAGAGAGGGAGAGAGGAAGGGAAGAGAGGACGTAATAATGTGAATTTGAGT
TCATGAAAATTTTTCAATAAAATAATTTAATGTCAGGAGAATTAAGCCTAATAGTCTCCTAAATCATCCATCTCTTG
AGCTTCAGAGCAGTCCTCTGAATTAATGCCTACATGTTTGTAAAGGGTGTTCAGACTGAAGCCAAGATTCTACCTCT
AAAGAGATGCAATCTCAAATTTATCTGAAGACTGTACCTCTGCTCTCCATAAATTGACACCATGGCCCACTTAATGA
GGTTAAAAAAAAGCTAATTCTGAATGAAAATCTGAGCCCAGTGGAGGAAATATTAATGAACAAGGTGCAGACTGAAA
TATAAATTTTCTGTAATAATTATGCATATACTTTAGCAAAGTTCTGTCTATGTTGACTTTATTGCTTTTGGTAAGAA
ATACAACTTTTTAAAGTGAACTAAACTATCCTATTTCCAAACTATTTTGTGTGTGTGCGGTTTGTTTCTATGGGTTC
TGGTTTTCTTGGAGCATTTTTATTTCATTTTAATTAATTAATTCTGAGAGCTGCTGAGTTGTGTTTACTGAGAGATT
GTGTATCTGCGAGAGAAGTCTGTAGCAAGTAGCTAGACTGTGCTTGACCTAGGAACATATACAGTAGATTGCTAAAA
TGTCTCACTTGGGGAATTTTAGACTAAACAGTAGAGCATGTATAAAAATACTCTAGTCAAGTGCTGCTTTTGAAACA
AATGATAAAACCACACTCCCATAGATGAGTGTCATGATTTTCATGGAGGAAGTTAATATTCATCCTCTAAGTATACC
CAGACTAGGGCCATTCTGATATAAAACATTAGGACTTAAGAAAGATTAATAGACTGGAGTAAAGGAAATGGACCTCT
GTCTCTCTCGCTGTCTCTTTTTTGAGGACTTGTGTGTGTGTGTGTGTGTGTGTGTGTGTGTGTTGTGGTCAGTGGGG
CTGGAATAAAAGTAGAATAGACCTGCACCTGCTGTGGCATCCATTCACAGAGTAGAAGCAAGCTCACAATAGTGAAG
ATGTCAGTAAGCTTGAATAGTTTTTCAGGAACTTTGAATGCTGATTTAGATTTGAAACTGAGGCTCTGACCATAACC
AAATTTGCACTATTTATTGCTTCTTGAAACTTATTTGCCTGGTATGCCTGGGCTTTTGATGGTCTTAGTATAGCTTG
CAGCCTTGTCCCTGCAGGGTATTATGGGTAATAGAAAGAAAAGTCTGCGTTACACTCTAGTCACACTAAGTAACTAC
CATTGGAAAAGCAACCCCTGCCTTGAAGCCAGGATGATGGTATCTGCAGCAGTTGCCAACACAAGAGAAGGATCCAT
AGTTCATCATTTAAAAAAGAAAACAAAATAGAAAAAGGAAAACTATTTCTGAGCATAAGAAGTTGTAGGGTAAGTCT
TTAAGAAGGTGACAATTTCTGCCAATCAGGATTTCAAAGCTCTTGCTTTGACAATTTTGGTCTTTCAGAATACTATA
AATATAACCTATATTATAATTTCATAAAGTCTGTGCATTTTCTTTGACCCAGGATATTTGCAAAAGACATATTCAAA
CTTCCGCAGAACACTTTATTTCACATATACATGCCTCTTATATCAGGGATGTGAAACAGGGTCTTGAAAACTGTCTA
AATCTAAAACAATGCTAATGCAGGTTTAAATTTAATAAAATAAAATCCAAAATCTAACAGCCAAGTCAAATCTGTAT
GTTTTAACATTTAAAATATTTTAAAGACGTCTTTTCCCAGGATTCAACATGTGAAATCTTTTCTCAGGGATACACGT
GTGCCTAGATCCTCATTGCTTTAGTTTTTTACAGAGGAATGAATATAAAAAGAAAATACTTAAATTTTATCCCTCTT
ACCTCTATAATCATACATAGGCATAATTTTTTAACCTAGGCTCCAGATAGCCATAGAAGAACCAAACACTTTCTGCG
TGTGTGAGAATAATCAGAGTGAGATTTTTTCACAAGTACCTGATGAGGGTTGAGACAGGTAGAAAAAGTGAGAGATC
TCTATTTATTTAGCAATAATAGAGAAAGCATTTAAGAGAATAAAGCAATGGAAATAAGAAATTTGTAAATTTCCTTC
TGATAACTAGAAATAGAGGATCCAGTTTCTTTTGGTTAACCTAAATTTTATTTCATTTTATTGTTTTATTTTATTTT
ATTTTATTTTATTTTGTGTAATCGTAGTTTCAGAGTGTTAGAGCTGAAAGGAAGAAGTAGGAGAAACATGCAAAGTA
AAAGTATAACACTTTCCTTACTAAACCGACTGGGTTTCCAGGTAGGGGCAGGATTCAGGATGACTGACAGGGCCCTT
AGGGAACACTGAGACCCTACGCTGACCTCATAAATGCTTGCTACCTTTGCTGTTTTAATTACATCTTTTAATAGCAG
GAAGCAGAACTCTGCACTTCAAAAGTTTTTCCTCACCTGAGGAGTTAATTTAGTACAAGGGGAAAAAGTACAGGGGG
ATGGGAGAAAGGCGATCACGTTGGGAAGCTATAGAGAAAGAAGAGTAAATTTTAGTAAAGGAGGTTTAAACAAACAA
AATATAAAGAGAAATAGGAACTTGAATCAAGGAAATGATTTTAAAACGCAGTATTCTTAGTGGACTAGAGGAAAAAA
ATAATCTGAGCCAAGTAGAAGACCTTTTCCCCTCCTACCCCTACTTTCTAAGTCACAGAGGCTTTTTGTTCCCCCAG
ACACTCTTGCAGATTAGTCCAGGCAGAAACAGTTAGATGTCCCCAGTTAACCTCCTATTTGACACCACTGATTACCC
CATTGATAGTCACACTTTGGGTTGTAAGTGACTTTTTATTTATTTGTATTTTTGACTGCATTAAGAGGTCTCTAGTT
TTTTATCTCTTGTTTCCCAAAACCTAATAAGTAACTAATGCACAGAGCACATTGATTTGTATTTATTCTATTTTTAG
ACATAATTTATTAGCATGCATGAGCAAATTAAGAAAAACAACAACAAATGAATGCATATATATGTATATGTATGTGT
GTATATATACACATATATATATATATTTTTTTTCTTTTCTTACCAGAAGGTTTTAATCCAAATAAGGAGAAGATATG
CTTAGAACTGAGGTAGAGTTTTCATCCATTCTGTCCTGTAAGTATTTTGCATATTCTGGAGACGCAGGAAGAGATCC
ATCTACATATCCCAAAGCTGAATTATGGTAGACAAAGCTCTTCCACTTTTAGTGCATCAATTTCTTATTTGTGTAAT
AAGAAAATTGGGAAAACGATCTTCAATATGCTTACCAAGCTGTGATTCCAAATATTACGTAAATACACTTGCAAAGG
AGGATGTTTTTAGTAGCAATTTGTACTGATGGTATGGGGCCAAGAGATATATCTTAGAGGGAGGGCTGAGGGTTTGA
AGTCCAACTCCTAAGCCAGTGCCAGAAGAGCCAAGGACAGGTACGGCTGTCATCACTTAGACCTCACCCTGTGGAGC
CACACCCTAGGGTTGGCCAATCTACTCCCAGGAGCAGGGAGGGCAGGAGCCAGGGCTGGGCATAAAAGTCAGGGCAG
AGCCATCTATTGCTTACATTTGCTTCTGACACAACTGTGTTCACTAGCAACCTCAAACAGACACCATGGTGCACCTG
ACTCCTGAGGAGAAGTCTGCCGTTACTGCCCTGTGGGGCAAGGTGAACGTGGATGAAGTTGGTGGTGAGGCCCTGGG
CAGGTTGGTATCAAGGTTACAAGACAGGTTTAAGGAGACCAATAGAAACTGGGCATGTGGAGACAGAGAAGACTCTT
GGGTTTCTGATAGGCACTGACTCTCTCTGCCTATTGGTCTATTTTCCCACCCTTAGGCTGCTGGTGGTCTACCCTTG
GACCCAGAGGTTCTTTGAGTCCTTTGGGGATCTGTCCACTCCTGATGCTGTTATGGGCAACCCTAAGGTGAAGGCTC
ATGGCAAGAAAGTGCTCGGTGCCTTTAGTGATGGCCTGGCTCACCTGGACAACCTCAAGGGCACCTTTGCCACACTG
AGTGAGCTGCACTGTGACAAGCTGCACGTGGATCCTGAGAACTTCAGGGTGAGTCTATGGGACCCTTGATGTTTTCT
TTCCCCTTCTTTTCTATGGTTAAGTTCATGTCATAGGAAGGGGAGAAGTAACAGGGTACAGTTTAGAATGGGAAACA
GACGAATGATTGCATCAGTGTGGAAGTCTCAGGATCGTTTTAGTTTCTTTTATTTGCTGTTCATAACAATTGTTTTC
TTTTGTTTAATTCTTGCTTTCTTTTTTTTTCTTCTCCGCAATTTTTACTATTATACTTAATGCCTTAACATTGTGTA
TAACAAAAGGAAATATCTCTGAGATACATTAAGTAACTTAAAAAAAAACTTTACACAGTCTGCCTAGTACATTACTA
TTTGGAATATATGTGTGCTTATTTGCATATTCATAATCTCCCTACTTTATTTTCTTTTATTTTTAATTGATACATAA
TCATTATACATATTTATGGGTTAAAGTGTAATGTTTTAATATGTGTACACATATTGACCAAATCAGGGTAATTTTGC
ATTTGTAATTTTAAAAAATGCTTTCTTCTTTTAATATACTTTTTTGTTTATCTTATTTCTAATACTTTCCCTAATCT
CTTTCTTTCAGGGCAATAATGATACAATGTATCATGCCTCTTTGCACCATTCTAAAGAATAACAGTGATAATTTCTG
GGTTAAGGCAATAGCAATATTTCTGCATATAAATATTTCTGCATATAAATTGTAACTGATGTAAGAGGTTTCATATT
GCTAATAGCAGCTACAATCCAGCTACCATTCTGCTTTTATTTTATGGTTGGGATAAGGCTGGATTATTCTGAGTCCA
AGCTAGGCCCTTTTGCTAATCATGTTCATACCTCTTATCTTCCTCCCACAGCTCCTGGGCAACGTGCTGGTCTGTGT
GCTGGCCCATCACTTTGGCAAAGAATTCACCCCACCAGTGCAGGCTGCCTATCAGAAAGTGGTGGCTGGTGTGGCTA
ATGCCCTGGCCCACAAGTATCACTAAGCTCGCTTTCTTGCTGTCCAATTTCTATTAAAGGTTCCTTTGTTCCCTAAG
TCCAACTACTAAACTGGGGGATATTATGAAGGGCCTTGAGCATCTGGATTCTGCCTAATAAAAAACATTTATTTTCA
TTGCAATGATGTATTTAAATTATTTCTGAATATTTTACTAAAAAGGGAATGTGGGAGGTCAGTGCATTTAAAACATA
AAGAAATGAAGAGCTAGTTCAAACCTTGGGAAAATACACTATATCTTAAACTCCATGAAAGAAGGTGAGGCTGCAAA
CAGCTAATGCACATTGGCAACAGCCCTGATGCCTATGCCTTATTCATCCCTCAGAAAAGGATTCAAGTAGAGGCTTG
ATTTGGAGGTTAAAGTTTTGCTATGCTGTATTTTACATTACTTATTGTTTTAGCTGTCCTCATGAATGTCTTTTCAC
TACCCATTTGCTTATCCTGCATCTCTCAGCCTTGACTCCACTCAGTTCTCTTGCTTAGAGATACCACCTTTCCCCTG
AAGTGTTCCTTCCATGTTTTACGGCGAGATGGTTTCTCCTCGCCTGGCCACTCAGCCTTAGTTGTCTCTGTTGTCTT
ATAGAGGTCTACTTGAAGAAGGAAAAACAGGGGGCATGGTTTGACTGTCCTGTGAGCCCTTCTTCCCTGCCTCCCCC
ACTCACAGTGACCCGGAATCTGCAGTGCTAGTCTCCCGGAACTATCACTCTTTCACAGTCTGCTTTGGAAGGACTGG
GCTTAGTATGAAAAGTTAGGACTGAGAAGAATTTGAAAGGGGGCTTTTTGTAGCTTGATATTCACTACTGTCTTATT
ACCCTATCATAGGCCCACCCCAAATGGAAGTCCCATTCTTCCTCAGGATGTTTAAGATTAGCATTCAGGAAGAGATC
AGAGGTCTGCTGGCTCCCTTATCATGTCCCTTATGGTGCTTCTGGCTCTGCAGTTATTAGCATAGTGTTACCATCAA
CCACCTTAACTTCATTTTTCTTATTCAATACCTAGGTAGGTAGATGCTAGATTCTGGAAATAAAATATGAGTCTCAA
GTGGTCCTTGTCCTCTCTCCCAGTCAAATTCTGAATCTAGTTGGCAAGATTCTGAAATCAAGGCATATAATCAGTAA
TAAGTGATGATAGAAGGGTATATAGAAGAATTTTATTATATGAGAGGGTGAAACCTAAAATGAAATGAAATCAGACC
CTTGTCTTACACCATAAACAAAAATAAATTTGAATGGGTTAAAGAATTAAACTAAGACCTAAAACCATAAAAATTTT
TAAAGAAATCAAAAGAAGAAAATTCTAATATTCATGTTGCAGCCGTTTTTTGAATTTGATATGAGAAGCAAAGGCAA
CAAAAGGAAAAATAAAGAAGTGAGGCTACATCAAACTAAAAAATTTCCACACAAAAAAGAAAACAATGAACAAATGA
AAGGTGAACCATGAAATGGCATATTTGCAAACCAAATATTTCTTAAATATTTTGGTTAATATCCAAAATATATAAGA
AACACAGATGATTCAATAACAAACAAAAAATTAAAAATAGGAAAATAAAAAAATTAAAAAGAAGAAAATCCTGCCAT
TTATGCGAGAATTGATGAACCTGGAGGATGTAAAACTAAGAAAAATAAGCCTGACACAAAAAGACAAATACTACACA
ACCTTGCTCATATGTGAAACATAAAAAAGTCACTCTCATGGAAACAGACAGTAGAGGTATGGTTTCCAGGGGTTGGG
GGTGGGAGAATCAGGAAACTATTACTCAAAGGGTATAAAATTTCAGTTATGTGGGATGAATAAATTCTAGATATCTA
ATGTACAGCATCGTGACTGTAGTTAATTGTACTGTAAGTATATTTAAAATTTGCAAAGAGAGTAGATTTTTTTGTTT
TTTTAGATGGAGTTTTGCTCTTGTTGTCCAGGCTGGAGTGCAATGGCAAGATCTTGGCTCACTGCAACCTCCGCCTC
CTGGGTTCAAGCAAATCTCCTGCCTCAGCCTCCCGAGTAGCTGGGATTACAGGCATGCGACACCATGCCCAGCTAAT
TTTGTATTTTTAGTAGAGACGGGGTTTCTCCATGTTGGTCAGGCTGATCCGCCTCCTCGGCCACCAAAGGGCTGGGA
TTACAGGCGTGACCACCGGGCCTGGCCGAGAGTAGATCTTAAAAGCATTTACCACAAGAAAAAGGTAACTATGTGAG
ATAATGGGTATGTTAATTAGCTTGATTGTGGTAATCATTTCACAAGGTATACATATATTAAAACATCATGTTGTACA
CCTTAAATATATACAATTTTTATTTGTGAATGATACCTCAATAAAGTTGAAGAATAATAAAAAAGAATAGACATCAC
ATGAATTAAAAAACTAAAAAATAAAAAAATGCATCTTGATGATTAGAATTGCATTCTTGATTTTTCAGATACAAATA
TCCATTTGACTGTTTACTCTTTTCCAAAACAATACAATAAATTTTAGCACTTTATCTTCATTTTCCCCTTCCCAATC
TATAATTTTATATATATATATTTTAGATATTTTGTATAGTTTTACTCCCTAGATTTTCTAGTGTTATTATTAAATAG
TGAAGAAATGTTTACACTTATGTACAAAATGTTTTGCATGCTTTTCTTCATTTCTAACATTCTCTCTAAGTTTATTC
TATTTTTTCCTGATTATCCTTAATATTATCTCTTTCTGCTGGAAATATATTGTTACTTTTGGTTTATCTAAAAATGG
CTTCATTTTCTTCATTCTAAAATCATGTTAAATTAATACCACTCATGTGTAAGTAAGATAGTGGAATAAATAGAAAT
CCAAAAACTAAATCTCACAAAATATAATAATGTGATATATAAAAATATAGCTTTTAAATTTAGCTTGGAAATAAAAA
ACAAACAGTAATTGAACAACTATACTTTTTGAAAAGAGTAAAGTGAAATGCTTAACTGCATATACCACAATCGATTA
CACAATTAGGTGTGAAGGTAAAATTCAGTCACGAAAAAACTAGAATAAAAATATGGGAAGACATGTATATAATCTTA
GAGATAACAGTGTTATTTAATTATCAACCCAAAGTAGAAACTATCAAGGGAGAAATAAATTCAGTCAACAATAAAAG
CATTTAAGAAGTTATTCTAGGCTGGGAGCGGTGGCTCACACCTGCAATTGCAGCACTTTGGGAGGCCTAGACAGGCG
GATCACGACGTCAGGAGTTCAAGATCAGCCTGGCCAACATAGTGAAACCTCATCGCTACTAAAAATATAAAAACTTA
GCCTGGCGTGGTGGCAGGCATGTGTAATCCCAGCAATTTGGGAGGCTGAGGCAGGAGAATCGCTTGATCCTGGGAGG
CAGAGGTTGCAGTGAGCCAAGATTGTGCCACTGCATTCCAGCCCAGGTGACAGCATGAGACTCCGTCACAAAAAAAA
AAGAAAAAAAAGGGGGGGGGGGGCGGTGGAGCCAAGATGACCGAATAGGAACAGCTCCAGTCTATAGCTCCCATCGT
GAGTGACGCAGAAGACGGGTGATTTCTGCATTTCCAACTGAGGTACCAGGTTCATCTCACAGGGAAGTGCCAGGCAG
TGGGTGCAGGACAGTAGTGCAGTGCACTGTGCATGAGCCGAAGCAGGGCGAGGCATCACCTCACCCGGGAAGCACAA
GGGGTCAGGGAATTCCCTTTCCTAGTCAAAGAAAAGGGTGACAGATGGCACCTGGAAAATCGGGTCACTCCCGCCCT
AATACTGCGCTCTTCCAACAAGCTTAACAAATGGCACACCAGGAGATTATATCCCATGCCTGGCTCAGAGGGTCCTA
CGCCCATGGAGCCTCGCTCATTGCTAGCACAGCAGTCTGAGGTCAAACTGCAAGGTGGCAGTGAGGCTGGGGGAGGG
GTGCCCACCATTGTCCAGGCTTGAGCAGGTAAACAAAGCCGCCTGGAAGCTCGAACTGGGTGGAGCCCACCACAGCT
CAAGGAGGCCTGCCTGCCTCTGTAGGCTCCACCTCTAGGGGCAGGGCACAGACAAACAAAAGACAACAAGAACCTCT
GCAGACTTAAATGTCCCTGTCTGACAGCTTTGAAGAGAGTAGTGGTTCTCCCAGCACATAGCTTCAGATCTGAGAAC
AGGCAGACTGCCTCCTCAAGTGGGTCCCTGACCCCCGAGTAGCCTAACTGGGAGGCATCCCCCAGTAGGGCGGACTG
ACACCTCACATGGCTGGTACTCCTCTAAGACAAAACTTCCAGAGGAATGATCAGGCAGCAGCATTTGCGGTTCACCA
ATATCCACTGTTCTGCAGCCACCGCTGCTGATACCCAGGAAAACAGCATCTGGAGTGGACCTCCAGTAAACTCCAAC
AGACCTGCAGCTGAGGGTCCTGACTGTTAGAAGGAAAACTAACAAACAGAAAGGACATCCACACCAAAAACCCATCT
GTACATCACCATCATCAAAGACCAAAGGTAGATAAAACCATAAAGATGGGGAAAAAGCAGAGCAGAAAAACTGGACA
CTCTAAAAATGAGAGTGCCTCTCCTTCTCCAAAGTAACGCAGCTCCTCACCAGCAATGGAACAAAGCTGGGCAGAGA
ATGACTTTGACGAGTTGAGAGAGGAAGGCTTCAGAAGATCAAACTACTCCAAGCTAAAGGAGGAAGTTCGAACAAAC
GGCAAAGAAGTAAAAAACTTTGAAAAAAAATTAGATGAATGGATAACTAGAATAACCAATGCACAGAAGTCCTTAAA
GGACCTGATGGAGCTGAAAACCAAGGCAGGAGAACTACGTGACAAATACACAAGCCTCAGTAACCGATGAGATCAAC
TGGAAGAAAGGGTATCAATGACGGAAGATGAAATGAATGAAATGAAGCATGAAGAGAAGTTTAGAGAAAAAAGAATA
AAAAGAAACGAACAAAGCCTCCAAGAAATATGGGACTATGTGAAAAGACCAAATCTACATCTAATTGGTGTAGCTGA
AAGTGATGGGGAGAATGGAACCAAGTTGGAAAACACTCTGCAGGATATTATCCAGGAGAACTTCCCCAATCTAGCAA
GGCAGCCCAAATTCACATTCAGGAAATACAGAGAACGCCACAAAGATACTCCTAGAGAAAAGCAACTCCAAGACACA
TAACTGACAGATTCACCAAAGTTGAAATGAAGGAAAAAATGTTAAGGGCAGCCAGAGAGAAAGGTCGGGTTACCCAC
AAAGGGAAGCCCATCAGACTAACAGCTGATCTATCGGCAGAAACTCTACAAGCCAGAAGAAAGTGGGGGCCAATATT
CAACATTGTTAAAGAAAAGAATTTTCGGCCCAGAATTTCATATCCAGCCAAACTAAGCTTCATAAGCATTGGAGAAA
TAAAATCCTTTACAGACAAGCAAATGCTGAGAGATTTTGTCACCACCAGGCCTGCCCTACAAGAGCTCCTGAAGGAA
GCACTAAACATGGAAAGGAACAACTAGTATCAGCCACTGCAAAAACATGCCAAATTGTAAACGACCATCAAGGCTAG
GAAGAAACTGCATCAAGGAGCAAAATAACCAGCTAACATCATAATGACAGGATCAAATTCATACATAACAATACTCA
CCTTAAATGTAAATAGGCTAAATGCTCCAATTAAAAGACACAGACTGGCAAATTGGATAAGGAGTCAAGACCCATCT
GTCGTTATGTATTCAGGAAACCCATCTCACGTGCAGAGACACACATAGGCTCGAAATAAAAGGATGGAGGAATATCT
ACCAAGCAAATGGAAAACAAAAAAAGGCAGGGGTTGCAATCCTAGTCTCTGATAAAACAGATTTTAAACCAACAAAG
ATCAAAAGAGACAAAGAAGGCCATTACATAATGGCAAAGGGATCTATTCAAGAAGAAGAACTAACTATACTAAATAT
ATATGCACCCAATACAGGAGCACCCAGATTCATAAAACAAGTCCTGAGTGACCTACAAAGAGACTTAGATGCCCACA
CAATAATAATGGGAGACTTTAACACCCCACTGTCAACATTAGACAGATCAACGAGACAGAAAGTTAACAAGGATATC
CAGGAATTGGACTCAGCTCTGCACCAAGCAGACCTAATAGACATCTACAGAACTCTCCACCCCAAATCAACAGAATA
TACATTCTTTTCAGCACCACACCACACCTATTCCAAAACTGACCACATAGTTGGAAGTAAAGCTCTCCTCAGCAAAT
GTAAAAGAACAGAAACTATAACAAACTGTCTCTCAGACCACAGTGCAATCAAACTAGAACTCAGGATTAAGAAACTC
ACTCAAAACCACTCAGCTACATGGAAACTGAACAGCCTGCTCCTGAATGACTACTGGGTACATAACAAAATGAAGGC
AGAAATAAAGATGTTCTTTGAAACAACGAGAACAAAGACACAACACACCAGAATCTCTGAGACACATTCAAAGCAGT
GTGTAGAGGGAAATTTATAGCACTAAATGCCCACAAGGGAAAGCAGGAAAGATCTAAAATTGACACCCTAACATCAC
AATTAAAAAACTAGAGAAGCAGGAGCAAACACATTCAAAAGCTAACAGAAGACAAGAAATAACTAAGATCAGAGCAG
AAGTGAAGAAGATAGAGACACAAAAAACCCTTCAAAAAAATCAATGAATCCAGAAGCTGTTTTTTTGAAAAGATCAA
CAAAATTGATAGACTGCTAGCAAGACTAATAAAGAAGAAAGGGGAGAAGAATCAAATAGACGCAATAAAAAATGACA
CGGGGTATCACCACTGATCCCACAGAAATACAAACTACCGTCAGAGAATACTATAAACACCTCTACGCAAATAAACT
AGAAAATCTAGAAGAAATGGATAAATTCCTCGACACATACACTCTGCCAAGACTAAACCAGGAAGAAGTTGTATCTC
TGAATAGACCAATAACAGGCTCTGAAATTGAGGCAATAATTAATAGCTTATCAACCAAAAAAAGTCCGGGACCAGTA
GGATTCATAGCCGAATTCTACCAGAGGTACAAGGAGGAGCTGGTACCATTCCTTCTGAAACTATTCCAATCAATAGA
AAAAGAGGGAATCCTCCCTAACTCATTTTATGAGGCCAGCATCATCCTGATACCAAAGCCTGACAGAGACACAACAA
AAAAAGAGAATGTTACACCAATATCCTTGATGAACATCGATGCAAAAATCCTCAATAAAATACTGGCAAACTGAATC
CAGCAGCACATCAAAAAGCTTATCCTCCATGATCAAGTGGGCTTCATCCCTGCCATGCAAGGCTGGTTCAACATACG
AAATCAATAAACATAATCCAGCATATAAACAGAACCAAAGACACAAACCATATGATTATCTCAATAGATGCAGAAAA
GGCCTTTGACAAAATTCAACAATGCTTCATGCTAAAAACTCTCAATAAATTAGGTATTGATGGGACATATCTCAAAA
TAATAAGAGCTATCTATGACAAACCCACAGCCAATATCATACTGAGTGGACAAAAACTGGAAGCATTCCCTTTGAAA
ACTGGCACAAGGCAGGGATGCCCTCTCTCACCACTCCTATTCAACATAGTGTTGGAAGTTCTGGCCAGGGCAATCAG
GCAGGAGAAGGAAATAAAGGGCATTCAATTAGGAAAAGAGGAAGGTGAAATTGTCCCTGTTTGCAGATGACATGATT
GTATATCTAGAAAACCCCATTGTCTCAGCCCAAAATCTCCTTAAGCTGATAAGCAACTTCAGCAAAGTCTCAGGATA
TAAAATCAGTGTGCAAAAATCACAAGTATTCCTATGCACCAATAACAGACAAACAGAGAGCCAAATCATGAGTGAAC
TCCCATTCACAATTGCTTCAAAGAGAATAAAATACCTAGGAATCCAACTTACAAGGGATGTGAAGGACCTCTTCAAG
GAGAACTACAAACCACTGCTCAATGAAATAAAAGAGGATACAAACAAATGGAAGAACATTCCATGCTTATGGGTAGG
AAGAATCATATCGTGAAAATGGTCATACTGCCCAAGGTAATTTATAGATTCAATGCCATCCCCATCAAGCTACCAAT
GACTTTCTTCACAGAACTGGAAAAAACTACTTTAAAGTTCATATGGAATCAAAAAAGAGCCCACATCACCAAGGCAA
TCCTAAGCCAAAAGAACAAAGCTGGAGGCATCACGCTACCTGACTTCAAACTATACTACAATGCTACGGTAACCAAA
ACAGCATGGTACTGGTACCAAAACAGAGATCTAGACCAATGGAACAGAACAGAGCCCTCAGAAATAATGCCGCATAT
CTACAACTATCCGATCTTTGACAAACCTGAGAGAAACAAGCAATGGGGAAAGGATTCCCTATTTAATAAATGGTGCT
GGGAAAACTGGCTAGCCATATGTAGAAAGCTGAAACTGGATCCTTCCTTACACCTTATACAAAAATTAATTCAAGAT
GGATTAAAGACTTAAACATTAGACCTAAAACCATAAAAACCCTAGAAAAAAACCTAGGCAATACCATTCAGGACATA
GGCATGGGCAAGGACTTCATGTCTAAAACACCAAAACGAATGGCAACAAAAGACAAAATGGACAAACGGGATCTAAT
TAAACTAAAGAGCTTCTGCACAGCTAAAGAAACTACCATCAGAGTGAACAGGCAACCTACAAAATGGGAGAAAATTT
TTGCAATCTACTCATCTGACAAAGGGCTAATATCCAGAATCTACAATGAACTCAAACAAATTTACAAGAAAAAACAA
ACAACCCCATCAAAAAGTGGGCAAAGGATATGAACAGACACTTCTCAAAAGAAGACATTTATGTAATCAAAAAACAC
ATGAAAAAATGCTCATCATCACTAGCCATCAGAGAAATGCAAATCAAAACCACAATGAGATACCATCTCACACCAGT
TAGAATGGCGATCATTAAAAAGTCAGGAAACAACAGGTGCTGGAGAGGATGTGGAGAAACAGGAACAACTTTTACAC
TGTTGGTGGGACTGTAAACTAGTTCAACCATTGCGGAAGTCAGTGTGGCAATTCCTCAGGAATCTAGAACTAGAAAT
ACCATTTGACCCAGCCATCCCATTACTGGGTAGATACCCAAAGGATTATAAATCATGCTGCTATAAAGACACATGCA
CACGTATGTTTATTGCAGCACTATTCACAATAGCAAAGACTTGGAACCAACCCAAATGTCCAACAACGATAGATTGG
ATTAAGAAAATGTGGCACATATACACCATGGAATACTATGCAGCCATAAAAAATGATGAGTTCATGTCCTTTGTAGG
GACATGGATGAAGCTGGAAACTATCATTCTCAGCAAACTATCACAAGGACAATAAACCAAACACCGCATGTTCTCAC
TCATAGGTGGGAATTGAACAATGAGAACACATGGACACATGAAGAGGAACATCACACTCTGGGGACTGTTATGGGGT
GGGGGGCAGGGGCAGGGATAGCACTAGGAGATATACCTAATGCTAAATGACGAGTTAATGGGTGCAGCACACCAACA
TGGCACATGTATACATATATAACAAACCTGCCGTTGTGCACATGTACCCTAAAACTTGAAGTATAATAATAAAAAAA
AGTTATCCTATTAAAACTGATCTCACACATCCGTAGAGCCATTATCAAGTCTTTCTCTTTGAAACAGACAGAAATTT
AGTGTTTTCTCAGTCAGTTAAC

GenBank Accession No. U01317.1 (SEQ ID NO: 249)

GAATTCTAATCTCCCTCTCAACCCTACAGTCACCCATTTGGTATATTAAAGATGTGTTGTCTACTGTCTAGTATCCC
TCAAGTAGTGTCAGGAATTAGTCATTTAAATAGTCTGCAAGCCAGGAGTGGTGGCTCATGTCTGTAATTCCAGCACT
GGAGAGGTAGAAGTGGGAGGACTGCTTGAGCTCAAGAGTTTGATATTATCCTGGACAACATAGCAAGACCTCGTCTC
TACTTAAAAAAAAAAAAATTAGCCAGGCATGTGATGTACACCTGTAGTCCCAGCTACTCAGGAGGCCGAAATGGGAG
GATCCCTTGAGCTCAGGAGGTCAAGGCTGCAGTGAGACATGATCTTGCCACTGCACTCCAGCCTGGACAGCAGAGTG
AAACCTTGCCTCACGAAACAGAATACAAAAACAAACAAACAAAAAACTGCTCCGCAATGCGCTTCCTTGATGCTCTA
CCACATAGGTCTGGGTACTTTGTACACATTATCTCATTGCTGTTCGTAATTGTTAGATTAATTTTGTAATATTGATA
TTATTCCTAGAAAGCTGAGGCCTCAAGATGATAACTTTTATTTTCTGGACTTGTAATAGCTTTCTCTTGTATTCACC
ATGTTGTAACTTTCTTAGAGTAGTAACAATATAAAGTTATTGTGAGTTTTTGCAAACACAGCAAACACAACGACCCA
TATAGACATTGATGTGAAATTGTCTATTGTCAATTTATGGGAAAACAAGTATGTACTTTTTCTACTAAGCCATTGAA
ACAGGAATAACAGAACAAGATTGAAAGAATACATTTTCCGAAATTACTTGAGTATTATACAAAGACAAGCACGTGGA
CCTGGGAGGAGGGTTATTGTCCATGACTGGTGTGTGGAGACAAATGCAGGTTTATAATAGATGGGATGGCATCTAGC
GCAATGACTTTGCCATCACTTTTAGAGAGCTCTTGGGGACCCCAGTACACAAGAGGGGACGCAGGGTATATGTAGAC
ATCTCATTCTTTTTCTTAGTGTGAGAATAAGAATAGCCATGACCTGAGTTTATAGACAATGAGCCCTTTTCTCTCTC
CCACTCAGCAGCTATGAGATGGCTTGCCCTGCCTCTCTACTAGGCTGACTCACTCCAAGGCCCAGCAATGGGCAGGG
CTCTGTCAGGGCTTTGATAGCACTATCTGCAGAGCCAGGGCCGAGAAGGGGTGGACTCCAGAGACTCTCCCTCCCAT
TCCCGAGCAGGGTTTGCTTATTTATGCATTTAAATGATATATTTATTTTAAAAGAAATAACAGGAGACTGCCCAGCC
CTGGCTGTGACATGGAAACTATGTAGAATATTTTGGGTTCCATTTTTTTTTCCTTCTTTCAGTTAGAGGAAAAGGGG
CTCACTGCACATACACTAGACAGAAAGTCAGGAGCTTTGAATCCAAGCCTGATCATTTCCATGTCATACTGAGAAAG
TCCCCACCCTTCTCTGAGCCTCAGTTTCTCTTTTTATAAGTAGGAGTCTGGAGTAAATGATTTCCAATGGCTCTCAT
TTCAATACAAAATTTCCGTTTATTAAATGCATGAGCTTCTGTTACTCCAAGACTGAGAAGGAAATTGAACCTGAGAC
TCATTGACTGGCAAGATGTCCCCAGAGGCTCTCATTCAGCAATAAAATTCTCACCTTCACCCAGGCCCACTGAGTGT
CAGATTTGCATGCACTAGTTCACGTGTGTAAAAAGGAGGATGCTTCTTTCCTTTGTATTCTCACATACCTTTAGGAA
AGAACTTAGCACCCTTCCCACACAGCCATCCCAATAACTCATTTCAGTGACTCAACCCTTGACTTTATAAAAGTCTT
GGGCAGTATAGAGCAGAGATTAAGAGTACAGATGCTGGAGCCAGACCACCTGAGTGATTAGTGACTCAGTTTCTCTT
AGTAATTGTATGACTCAGTTTCTTCATCTGTAAAATGGAGGGTTTTTTAATTAGTTTGTTTTTGAGAAAGGGTCTCA
CTCTGTCACCCAAATGGGAGTGTAGTGGCAAAATCTCGGCTCACTGCAACTTGCACTTCCCAGGCTCAAGCGGTCCT
CCCACCTCAACATCCTGAGTAGCTGGAACCACAGGTACACACCACCATACCTCGCTAATTTTTTGTATTTTTGGTAG
AGATGGGGTTTCACATGTTACACAGGATGGTCTCAGACTCCGGAGCTCAAGCAATCTGCCCACCTCAGCCTTCCAAA
GTGCTGGGATTATAAGCATGATTACAGGAGTTTTAACAGGCTCATAAGATTGTTCTGCAGCCCGAGTGAGTTAATAC
ATGCAAAGAGTTTAAAGCAGTGACTTATAAATGCTAACTACTCTAGAAATGTTTGCTAGTATTTTTTGTTTAACTGC
AATCATTCTTGCTGCAGGTGAAAACTAGTGTTCTGTACTTTATGCCCATTCATCTTTAACTGTAATAATAAAAATAA
CTGACATTTATTGAAGGCTATCAGAGACTGTAATTAGTGCTTTGCATAATTAATCATATTTAATACTCTTGGATTCT
TTCAGGTAGATACTATTATTATCCCCATTTTACTACAGTTAAAAAAACTACCTCTCAACTTGCTCAAGCATACACTC
TCACACACACAAACATAAACTACTAGCAAATAGTAGAATTGAGATTTGGTCCTAATTATGTCTTTGCTCACTATCCA
ATAAATATTTATTGACATGTACTTCTTGGCAGTCTGTATGCTGGATGCTGGGGATACAAAGATGTTTAAATTTAAGC
TCCAGTCTCTGCTTCCAAAGGCCTCCCAGGCCAAGTTATCCATTCAGAAAGCATTTTTTACTCTTTGCATTCCACTG
TTTTTCCTAAGTGACTAAAAAATTACACTTTATTCGTCTGTGTCCTGCTCTGGGATGATAGTCTGACTTTCCTAACC
TGAGCCTAACATCCCTGACATCAGGAAAGACTACACCATGTGGAGAAGGGGTGGTGGTTTTGATTGCTGCTGTCTTC
AGTTAGATGGTTAACTTTGTGAAGTTGAAAACTGTGGCTCTCTGGTTGACTGTTAGAGTTCTGGCACTTGTCACTAT
GCCTATTATTTAACAAATGCATGAATGCTTCAGAATATGGGAATATTATCTTCTGGAATAGGGAATCAAGTTATATT
ATGTAACCCAGGATTAGAAGATTCTTCTGTGTGTAAGAATTTCATAAACATTAAGCTGTCTAGCAAAAGCAAGGGCT
TGGAAAATCTGTGAGCTCCTCACCATATAGAAAGCTTTTAACCCATCATTGAATAAATCCCTATAGGGGATTTCTAC
CCTGAGCAAAAGGCTGGTCTTGATTAATTCCCAAACTCATATAGCTCTGAGAAAGTCTATGCTGTTAACGTTTTCTT
GTCTGCTACCCCATCATATGCACAACAATAAATGCAGGCCTAGGCATGACTGAAGGCTCTCTCATAATTCTTGGTTG
CATGAATCAGATTATCAACAGAAATGTTGAGACAAACTATGGGGAAGCAGGGTATGAAAGAGCTCTGAATGAAATGG
AAACCGCAATGCTTCCTGCCCATTCAGGGCTCCAGCATGTAGAAATCTGGGGCTTTGTGAAGACTGGCTTAAAATCA
GAAGCCCCATTGGATAAGAGTAGGGAAGAACCTAGAGCCTACGCTGAGCAGGTTTCCTTCATGTGACAGGGAGCCTC
CTGCCCCGAACTTCCAGGGATCCTCTCTTAAGTGTTTCCTGCTGGAATCTCCTCACTTCTATCTGGAAATGGTTTCT
CCACAGTCCAGCCCCTGGCTAGTTGAAAGAGTTACCCATGCAGAGGCCCTCCTAGCATCCAGAGACTAGTGCTTAGA
TTCCTACTTTCAGCGTTGGACAACCTGGATCCACTTGCCCAGTGTTCTTCCTTAGTTCCTACCTTCGACCTTGATCC
TCCTTTATCTTCCTGAACCCTGCTGAGATGATCTATGTGGGGAGAATGGCTTCTTTGAGAAACATCTTCTTCGTTAG
TGGCCTGCCCCTCATTCCCACTTTAATATCCAGAATCACTATAAGAAGAATATAATAAGAGGAATAACTCTTATTAT
AGGTAAGGGAAAATTAAGAGGCATACGTGATGGGATGAGTAAGAGAGGAGAGGGAAGGATTAATGGATGATAAAATC
TACTACTATTTGTTGAGACCTTTTATAGTCTAATCAATTTTGCTATTGTTTTCCATCCTCACGCTAACTCCATAAAA
AAACACTATTATTATCTTTATTTTGCCATGACAAGACTGAGCTCAGAAGAGTCAAGCATTTGCCTAAGGTCGGACAT
GTCAGAGGCAGTGCCAGACCTATGTGAGACTCTGCAGCTACTGCTCATGGGCCCTGTGCTGCACTGATGAGGAGGAT
CAGATGGATGGGGCAATGAAGCAAAGGAATCATTCTGTGGATAAAGGAGACAGCCATGAAGAAGTCTATGACTGTAA
ATTTGGGAGCAGGAGTCTCTAAGGACTTGGATTTCAAGGAATTTTGACTCAGCAAACACAAGACCCTCACGGTGACT
TTGCGAGCTGGTGTGCCAGATGTGTCTATCAGAGGTTCCAGGGAGGGTGGGGTGGGGTCAGGGCTGGCCACCAGCTA
TCAGGGCCCAGATGGGTTATAGGCTGGCAGGCTCAGATAGGTGGTTAGGTCAGGTTGGTGGTGCTGGGTGGAGTCCA
TGACTCCCAGGAGCCAGGAGAGATAGACCATGAGTAGAGGGCAGACATGGGAAAGGTGGGGGAGGCACAGCATAGCA
GCATTTTTCATTCTACTACTACATGGGACTGCTCCCCTATACCCCCAGCTAGGGGCAAGTGCCTTGACTCCTATGTT
TTCAGGATCATCATCTATAAAGTAAGAGTAATAATTGTGTCTATCTCATAGGGTTATTATGAGGATCAAAGGAGATG
CACACTCTCTGGACCAGTGGCCTAACAGTTCAGGACAGAGCTATGGGCTTCCTATGTATGGGTCAGTGGTCTCAATG
TAGCAGGCAAGTTCCAGAAGATAGCATCAACCACTGTTAGAGATATACTGCCAGTCTCAGAGCCTGATGTTAATTTA
GCAATGGGCTGGGACCCTCCTCCAGTAGAACCTTCTAACCAGCTGCTGCAGTCAAAGTCGAATGCAGCTGGTTAGAC
TTTTTTTAATGAAAGCTTAGCTTTCATTAAAGATTAAGCTCCTAAGCAGGGCACAGATGAAATTGTCTAACAGCAAC
TTTGCCATCTAAAAAAATCTGACTTCACTGGAAACATGGAAGCCCAAGGTTCTGAACATGAGAAATTTTTAGGAATC
TGCACAGGAGTTGAGAGGGAAACAAGATGGTGAAGGGACTAGAAACCACATGAGAGACACGAGGAAATAGTGTAGAT
TTAGGCTGGAGGTAAATGAAAGAGAAGTGGGAATTAATACTTACTGAAATCTTTCTATATGTCAGGTGCCATTTTAT
GATATTTAATAATCTCATTACATATGGTAATTCTGTGAGATATGTATTATTGAACATACTATAATTAATACTAATGA
TAAGTAACACCTCTTGAGTACTTAGTATATGCTAGAATCAAATTTAAGTTTATCATATGAGGCCGGGCACGGTGGCT
CATATATGGGATTACATGCCTGTAATCCCAGCACTTTGGGAGGCCAAGGCAATTGGATCACCTGAGGTCAGGAGTTC
CAGACCAGCCTGGCCAACATGGTGAAACCCCTTCTCTACTAAAAAATACAAAAAATCAGCCAGGTGTGGTGGCACGC
GTCTATAATCCCAGCTACTCAGGAGGCTGAGGCAGGAGAATCACTTGAACCCAGGAGGTGGAGGTTGCAGTGAGCTA
AGATTGCACCACTGCACTCCAGCCTAGGCGACAGAGTGAGACTCCATCTCAAAAAAAAAAAAAGAAGTTTATTATAT
GAATTAACTTAGTTTTACTCACACCAATACTCAGAAGTAGATTATTACCTCATTTATTGATGAGGAGCCCAATGTAC
TTGTAGTGTAGATCAACTTATTGAAAGCACAAGCTAATAAGTAGACAATTAGTAATTAGAAGTCAGATGGTCTGAGC
TCTCCTACTGTCTACATTACATGAGCTCTTATTAACTGGGGACTCGAAAATCAAAGACATGAAATAATTTGTCCAAG
CTTACAGAACCACCAAGTAGTAAGGCTAGGATGTAGACCCAGTTCTGCTACCTCTGAAGACAGTGTTTTTTCCACAG
CAAAACACAAACTCAGATATTGTGGATGCGAGAAATTAGAAGTAGATATTCCTGCCCTGTGGCCCTTGCTTCTTACT
TTTACTTCTTGGCGATTGGAAGTTGTGGTCCAAGCCACAGTTGCAGACCATACTTCCTCAACCATAATTGCATTTCT
TCAGGAAAGTTTGAGGGAGAAAAAGGTAAAGAAAAATTTAGAAACAACTTCAGAATAAAGAGATTTTCTCTTGGGTT
ACAGAGATTGTCATATGACAAATTATAAGCAGACACTTGAGAAAACTGAAGGCCCATGCCTGCCCAAATTACCCTTT
GACCCCTTGGTCAAGCTGCAACTTTGGTTAAAGGGAGTGTTTATGTGTTATAGTGTTCATTTACTCTTCTGGTCTAA
CCCATTGGCTCCGTCTTCATCCTGCAGTGACCTCAGTGCCTCAGAAACATACATATGTTTGTCTAGTTTAAGTTTGT
GTGAAATTCTAACTAGCGTCAAGAACTGAGGGCCCTAAACTATGCTAGGAATAGTGCTGTGGTGCTGTGATAGGTAC
ACAAGAAATGAGAAGAAACTGCAGATTCTCTGCATCTCCCTTTGCCGGGTCTGACAACAAAGTTTCCCCAAATTTTA
CCAATGCAAGCCATTTCTCCATATGCTAACTACTTTAAAATCATTTGGGGCTTCACATTGTCTTTCTCATCTGTAAA
AAGAATGGAAGAACTCATTCCTACAGAACTCCCTATGTCTTCCCTGATGGGCTAGAGTTCCTCTTTCTCAAAAATTA
GCCATTATTGTATTTCCTTCTAAGCCAAAGCTCAGAGGTCTTGTATTGCCCAGTGACATGCACACTGGTCAAAAGTA
GGCTAAGTAGAAGGGTACTTTCACAGGAACAGAGAGCAAAAGAGGTGGGTGAATGAGAGGGTAAGTGAGAAAAGACA
AATGAGAAGTTACAACATGATGGCTTGTTGTCTAAATATCTCCTAGGGAATTATTGTGAGAGGTCTGAATAGTGTTG
TAAAATAAGCTGAATCTGCTGCCTAACATTAACAGTCAAGAAATACCTCCGAATAACTGTACCTCCAATTATTCTTT
AAGGTAGCATGCAACTGTAATAGTTGCATGTATATATTTATCATAATACTGTAACAGAAAACACTTACTGAATATAT
ACTGTGTCCCTAGTTCTTTACACAATAAACTAATCTCATCCTCATAATTCTATTAGCTAATACATATTATCATCCTA
TATTTCAGAGACTTCAAGAAGTTAAGCAACTTGCTCAAGATCATCTAAGAAGTAGGTGGTATTTCTGGGCTCATTTG
GCCCCTCCTAATCTCTCATGGCAACATGGCTGCCTAAAGTGTTGATTGCCTTAATTCATCAGGGATGGGCTCATACT
CACTGCAGACCTTAACTGGCATCCTCTTTTCTTATGTGATCTGCCTGACCCTAGTAGAACTTATGAAATTTCTGATG
AGAAAGGAGAGAGGAGAAAGGCAGAGCTGACTGTGATGAGTGATGAAGGTGCCTTCTCATCTGGGTACCAGTGGGGC
CTCTAAGACTAAGTCACTCTGTCTCACTGTGTCTTAGCCAGTTCCTTACAGCTTGCCCTGATGGGAGATAGAGAATG
GGTATCCTCCAACAAAAAAATAAATTTTCATTTCTCAAGGTCCAACTTATGTTTTCTTAATTTTTAAAAAAATCTTG
ACCATTCTCCACTCTCTAAAATAATCCACAGTGAGAGAAACATTCTTTTCCCCCATCCCATAAATACCTCTATTAAA
TATGGAAAATCTGGGCATGGTGTCTCACACCTGTAATCCCAGCACTTTGGGAGGCTGAGGTGGGTGGACTGCTTGGA
GCTCAGGAGTTCAAGACCATCTTGGACAACATGGTGATACCCTGCCTCTACAAAAAGTACAAAAATTAGCCTGGCAT
GGTGGTGTGCACCTGTAATCCCAGCTATTAGGGTGGCTGAGGCAGGAGAATTGCTTGAACCCGGGAGGCGGAGGTTG
CAGTGAGCTGAGATCGTGCCACTGCACTCCAGCCTGGGGGACAGAGCACATTATAATTAACTGTTATTTTTTACTTG
GACTCTTGTGGGGAATAAGATACATGTTTTATTCTTATTTATGATTCAAGCACTGAAAATAGTGTTTAGCATCCAGC
AGGTGCTTCAAAACCATTTGCTGAATGATTACTATACTTTTTACAAGCTCAGCTCCCTCTATCCCTTCCAGCATCCT
CATCTCTGATTAAATAAGCTTCAGTTTTTCCTTAGTTCCTGTTACATTTCTGTGTGTCTCCATTAGTGACCTCCCAT
AGTCCAAGCATGAGCAGTTCTGGCCAGGCCCCTGTCGGGGTCAGTGCCCCACCCCCGCCTTCTGGTTCTGTGTAACC
TTCTAAGCAAACCTTCTGGCTCAAGCACAGCAATGCTGAGTCATGATGAGTCATGCTGAGGCTTAGGGTGTGTGCCC
AGATGTTCTCAGCCTAGAGTGATGACTCCTATCTGGGTCCCCAGCAGGATGCTTACAGGGCAGATGGCAAAAAAAAG
GAGAAGCTGACCACCTGACTAAAACTCCACCTCAAACGGCATCATAAAGAAAATGGATGCCTGAGACAGAATGTGAC
ATATTCTAGAATATATTATTTCCTGAATATATATATATATATATATACACATATACGTATATATATATATATATATA
TATTTGTTGTTATCAATTGCCATAGAATGATTAGTTATTGTGAATCAAATATTTATCTTGCAGGTGGCCTCTATACC
TAGAAGCGGCAGAATCAGGCTTTATTAATACATGTGTATAGATTTTTAGGATCTATACACATGTATTAATATGAAAC
AAGGATATGGAAGAGGAAGGCATGAAAACAGGAAAAGAAAACAAACCTTGTTTGCCATTTTAAGGCACCCCTGGACA
GCTAGGTGGCAAAAGGCCTGTGCTGTTAGAGGACACATGCTCACATACGGGGTCAGATCTGACTTGGGGTGCTACTG
GGAAGCTCTCATCTTAAGGATACATCTCAGGCCAGTCTTGGTGCATTAGGAAGATGTAGGCAACTCTGATCCTGAGA
GGAAAGAAACATTCCTCCAGGAGAGCTAAAAGGGTTCACCTGTGTGGGTAACTGTGAAGGACTACAAGAGGATGAAA
AACAATGACAGACAGACATAATGCTTGTGGGAGAAAAAACAGGAGGTCAAGGGGATAGAGAAGGCTTCCAGAAGAAT
GGCTTTGAAGCTGGCTTCTGTAGGAGTTCACAGTGGCAAAGATGTTTCAGAAATGTGACATGACTTAAGGAACTATA
CAAAAAGGAACAAATTTAAGGAGAGGCAGATAAATTAGTTCAACAGACATGCAAGGAATTTTCAGATGAATGTTATG
TCTCCACTGAGCTTCTTGAGGTTAGCAGCTGTGAGGGTTTTGCAGGCCCAGGACCCATTACAGGACCTCACGTATAC
TTGACACTGTTTTTTGTATTCATTTGTGAATGAATGACCTCTTGTCAGTCTACTCGGTTTCGCTGTGAATGAATGAT
GTCTTGTCAGCCTACTTGGTTTCGCTAAGAGCACAGAGAGAAGATTTAGTGATGCTATGTAAAAACTTCCTTTTTGG
TTCAAGTGTATGTTTGTGATAGAAATGAAGACAGGCTACATGATGCATATCTAACATAAACACAAACATTAAGAAAG
GAAATCAACCTGAAGAGTATTTATACAGATAACAAAATACAGAGAGTGAGTTAAATGTGTAATAACTGTGGCACAGG
CTGGAATATGAGCCATTTAAATCACAAATTAATTAGAAAAAAAACAGTGGGGAAAAAATTCCATGGATGGGTCTAGA
AAGACTAGCATTGTTTTAGGTTGAGTGGCAGTGTTTAAAGGGTGATATCAGACTAAACTTGAAATATGTGGCTAAAT
AACTAGAATACTCTTTATTTTTTCGTATCATGAATAGCAGATATAGCTTGATGGCCCCATGCTTGGTTTAACATCCT
TGCTGTTCCTGACATGAAATCCTTAATTTTTGACAAAGGGGCTATTCATTTTCATTTTATATTGGGCCTAGAAATTA
TGTAGATGGTCCTGAGGAAAAGTTTATAGCTTGTCTATTTCTCTCTCTAACATAGTTGTCAGCACAATGCCTAGGCT
ATAGGAAGTACTCAAAGCTTGTTAAATTGAATTCTATCCTTCTTATTCAATTCTACACATGGAGGAAAAACTCATCA
GGGATGGAGGCACGCCTCTAAGGAAGGCAGGTGTGGCTCTGCAGTGTGATTGGGTACTTGCAGGACGAAGGGTGGGG
TGGGAGTGGCTAACCTTCCATTCCTAGTGCAGAGGTCACAGCCTAAACATCAAATTCCTTGAGGTGCGGTGGCTCAC
TCCTGTAATCACAGCAGTTTGGGACGCCAAGGTGGGCAGATCACTTGAGGTCAGGAGTTGGACACCAGCCCAGCCAA
CATAGTGAAACCTGGTCTCTGCTTAAAAATATAAAAATTAGCTGGACGTGGTGACGGGAGCCTGTAATCCAACTACT
TGGGAGGCTGAGGCAGGAGAATCGCTTGAACCGGGGAGGTGGAGTTTGCACTGAGCAGAGATCATGCCATTGCACTC
CAGCCTCCAGAGCGAGACTCTGTCTAAAGAAAAACGAAAACAAACAAACAAACAAACAAACAAAACCCATCAAATTC
CCTGACCGAACAGAATTCTGTCTGATTGTTCTCTGACTTATCTACCATTTTCCCTCCTTAAAGAAACTGTGGAACTT
CCTTCAGCTAGAGGGGCCTGGCTCAGAAGCCTCTGGTCAGCATCCAAGAAATACTTGATGTCACTTTGGCTAAAGGT
ATGATGTGTAGACAAGCTCCAGAGATGGTTTCTCATTTCCATATCCACCCACCCAGCTTTCCAATTTTAAAGCCAAT
TCTGAGGTAGAGACTGTGATGAACAAACACCTTGACAAAATTCAACCCAAAGACTCACTTTGCCTAGCTTCAAAATC
CTTACTCTGACATATACTCACAGCCAGAAATTAGCATGCACTAGAGTGTGCATGAGTGCAACACACACACACACCAA
TTCCATATTCTCTGTCAGAAAATCCTGTTGGTTTTTCGTGAAAGGATGTTTTCAGAGGCTGACCCCTTGCCTTCACC
TCCAATGCTACCACTCTGGTCTAAGTCACTGTCACCACCACCTAAATTATAGCTGTTGACTCATAACAATCTTCCTG
CTTCTACCACTGCCCCACTACAATTTCTTCCCAATATACTATCCAAATTAGTCTTTTCAAAATGTAAGTCATATATG
GTCACCTCTTTGTTCAAAGTCTTCTGATAGTTTCCTATATCATTTATAATAAAACCAAATCCTTACAATTCTCTACA
ATAGTTGTTCATGCATATATTATGTTTATTACAGATACGCATATATATAGCTCTCATATAAATAAATATATATATTT
ATGTGTATGTGTGTAGAGTGTTTTTTCTTACAACTCTATGATGTAGGTATTATTAGTGTCCCAAATTTTATAATTTA
GGACTTCTATGATCTCATCTTTTATTCTCCCCTTCACCGAATCTCATCCTACATTGGCCTTATTGATATTCCTTGAA
AATTCTAAGCATCTTACATCTTTAGGGTATTTACATTTGCCATTCCCTATGCCCTAAATATTTAATCATAGTTTCAT
ATAAATGGGTTCCTCATCATCTATGGGTACTCTCTCAGGTGTTAACTTTATAGTGAGGACTTTCCTGCCATACTACT
TAAAGTAGCGATACCCTTTCACCCTGTCCTAATCACACTCTGGCCTTCATTTCAGTTTTTTTTTTTTCTCCATAGCA
CCTAATCTCATTGGTATATAACATGTTTCATTTGCTTATTTAATGTCAAGCTCTTTCCACTATCAAGTCCATGAAAA
CAGGAACTTTATTCCTCTATTCTGTTTTTGTGCTGTATTCTTAGCAATTTTACAATTTTGAATGAAATGAATGAGCA
GTCAAACACATATACAACTATAATTAAAAGGATGTATGCTGACACATCCACTGCTATGCACACACAAAGAAATCAGT
GGAGTAGAGCTGGAAGCGCTAAGCCTGCATAGAGCTAGTTAGCCCTCCGCAGGCAGAGCCTTGATGGGATTACTGAG
TTCTAGAATTGGACTCATTTGTTTTGTAGGCTGAGATTTGCTCTTGAAAACTTGTTCTGACCAAAATAAAAGGCTCA
AAAGATGAATATCGAAACCAGGGTGTTTTTTACACTGGAATTTATAACTAGAGCACTCATGTTTATGTAAGCAATTA
ATTGTTTCATCAGTCAGGTAAAAGTAAAGAAAAACTGTGCCAAGGCAGGTAGCCTAATGCAATATGCCACTAAAGTA
AACATTATTCCATAGGTGTCAGATATGGCTTATTCATCCATCTTCATGGGAAGGATGGCCTTGGCCTGGACATCAGT
GTTATGTGAGGTTCAAAACACCTCTAGGCTATAAGGCAACAGAGCTCCTTTTTTTTTTTTCTGTGCTTTCCTGGCTG
TCCAAATCTCTAATGATAAGCATACTTCTATTCAATGAGAATATTCTGTAAGATTATAGTTAAGAATTGTGGGAGCC
ATTCCGTCTCTTATAGTTAAATTTGAGCTTCTTTTATGATCACTGTTTTTTTAATATGCTTTAAGTTCTGGGGTACA
TGTGCCATGGTGGTTTGCTGCACCCATCAACCCGTCATCTACATTAGGTATTTCTCCTAATGCTATCCTTCCCCTAG
CCCCCCACCCCCAACAGGCCCCAGTGTGTGATGTTCCCCTCCCTGTGTCCATGGATCACTGGTTTTTTTTTTTTTTT
TTTTTTTTTTTTTAAAGTCTCAGTTAAATTTTTGGAATGTAATTTATTTTCCTGGTATCCTAGGACCTGCAAGTTAT
CTGGTCACTTTAGCCCTCACGTTTTGATGATAATCACATATTTGTAAACACAACACACACACACACACACACACACA
TATATATATATAAAACATATATATACATAAACACACATAACATATTTATCGGGCATTTCTGAGCAACTAACTCATGC
AGGACTCTCAAACACTAACCTATAGCCTTTTCTATGTATCTACTTGTGTAGAAACCAAGCGTGGGGACTGAGAAGGC
AATAGCAGGAGCATTCTGACTCTCACTGCCTTTGGCTAGGTCCCTCCCTCATCACAGCTCAGCATAGTCCGAGCTCT
TATCTATATCCACACACAGTTTCTGACGCTGCCCAGCTATCACCATCCCAAGTCTAAAGAAAAAAATAATGGGTTTG
CCCATCTCTGTTGATTAGAAAACAAAACAAAATAAAATAAGCCCCTAAGCTCCCAGAAAACATGACTAAACCAGCAA
GAAGAAGAAAATACAATAGGTATATGAGGAGACTGGTGACACTAGTGTCTGAATGAGGCTTGAGTACAGAAAAGAGG
CTCTAGCAGCATAGTGGTTTAGAGGAGATGTTTCTTTCCTTCACAGATGCCTTAGCCTCAATAAGCTTGCGGTTGTG
GAAGTTTACTTTCAGAACAAACTCCTGTGGGGCTAGAATTATTGATGGCTAAAAGAAGCCCGGGGGAGGGAAAAATC
ATTCAGCATCCTCACCCTTAGTGACACAAAACAGAGGGGGCCTGGTTTTCCATATTTCCTCATGATGGATGATCTCG
TTAATGAAGGTGGTCTGACGAGATCATTGCTTCTTCCATTTAAGCCTTGCTCACTTGCCAATCCTCAGTTTTAACCT
TCTCCAGAGAAATACACATTTTTTATTCAGGAAACATACTATGTTATAGTTTCAATACTAAATAATCAAAGTACTGA
AGATAGCATGCATAGGCAAGAAAAAGTCCTTAGCTTTATGTTGCTGTTGTTTCAGAATTTAAAAAAGATCACCAAGT
CAAGGACTTCTCAGTTCTAGCACTAGAGGTGGAATCTTAGCATATAATCAGAGGTTTTTCAAAATTTCTAGACATGA
GATTCAAAGCCCTGCACTTAAAATAGTCTCATTTGAATTAACTCTTTATATAAATTGAAAGCACATTCTGAACTACT
TCAGAGTATTGTTTTATTTCTATGTTCTTAGTTCATAAATACATTAGGCAATGCAATTTAATTAAAAAAACCCAAGA
ATTTCTTAGAATTTTAATCATGAAAATAAATGAAGGCATCTTTACTTACTCAAGGTCCCAAAAGGTCAAAGAAACCA
GGAAAGTAAAGCTATATTTCAGCGGAAAATGGGATATTTATGAGTTTTCTAAGTTGACAGACTCAAGTTTTAACCTT
CAGTGCCCATGATGTAGGAAAGTGTGGCATAACTGGCTGATTCTGGCTTTCTACTCCTTTTTCCCATTAAAGATCCC
TCCTGCTTAATTAACATTCACAAGTAACTCTGGTTGTACTTTAGGCACAGTGGCTCCCGAGGTCAGTCACACAATAG
GATGTCTGTGCTCCAAGTTGCCAGAGAGAGAGATTACTCTTGAGAATGAGCCTCAGCCCTGGCTCAAACTCACCTGC
AAACTTCGTGAGAGATGAGGCAGAGGTACACTACGAAAGCAACAGTTAGAAGCTAAATGATGAGAACACATGGACTC
ATAGAGGGAAACAACGCATACTGGGGCCTATCAGAGGGTGGAGGGTGAGAGAAGGAGAGGATCAGGAAAAATCACTA
ATGGATGCTAAGCGTAATACCTGAGTGATGAGATCATCTATACAACAAACCCCCTTGACATTCATTTATCTATGTAA
CAAACCTGCACATCCTGTACACGTACCCCTGAACTTAAAATAAAAGTTGAAAACAAGAAAGCAACAGTTTGAACACT
TGTTATGGTCTATTCTCTCATTCTTTACAATTACACTAGAAAATAGCCACAGGCTCCTGCAAGGCAGCCACAGAATT
TATGACTTGTGATATCCAAGTCATTCCTGGATAATGCAAAATCTAACACAAAATCTAGTAGAATCATTTGCTTACAT
CTATTTTTGTTCTGAGAATATAGATTTAGATACATAATGGAAGCAGAATAATTTAAAATCTGGCTAATTTAGAATCC
TAAGCAGCTCTTTTCCTATCAGTGGTTTACAAGCCTTGTTTATATTTTTCCTATTTTAAAAATAAAAATAAAGTAAG
TTATTTGTGGTAAAGAATATTCATTAAAGTATTTATTTCTTAGATAATACCATGAAAAACATTCAGTGAAGTGAAGG
GCCTACTTTACCCAACAAGAATCTAATTTATATAATTTTTCATACTAATAGCATCTAAGAACAGTACAATATTTGAC
TCTTCAGGTTAAACATATGTCATAAATTAGCCAGAAAGATTTAAGAAAATATTGGATGTTTCCTTGTTTAAATTAGG
CATCTTACAGTTTTTAGAATCCTGCATAGAACTTAAGAAATTACAAATGCTAAAGCAAACCCAAACAGGCAGGAATT
AATCTTCATCGAATTTGGGTGTTTCTTTCTAAAAGTCCTTTATACTTAAATGTCTTAAGACATACATAGATTTTATT
TTACTAATTTTAATTATACAGACAATAAATGAATATTCTTACTGATTACTTTTTCTGACTGTCTAATCTTTCTGATC
TATCCTGGATGGCCATAACACTTATCTCTCTGAACTTTGGGCTTTTAATATAGGAAAGAAAAGCAATAATCCATTTT
TCATGGTATCTCATATGATAAACAAATAAAATGCTTAAAAATGAGCAGGTGAAGCAATTTATCTTGAACCAACAAGC
ATCGAAGCAATAATGAGACTGCCCGCAGCCTACCTGACTTCTGAGTCAGGATTTATAAGCCTTGTTACTGAGACACA
AACCTGGGCCTTTCAATGCTATAACCTTTCTTGAAGCTCCTCCCTACCACCTTTAGCCATAAGGAAACATGGAATGG
GTCAGATCCCTGGATGCAAGCCAGGTCTGGAACCATAGGCAGTAAGGAGAGAAGAAAATGTGGGCTCTGCAACTGGC
TCCGAGGGAGCAGGAGAGAATCAACCCCATACTCTGAATCTAAGAGAAGACTGGTGTCCATACTCTGAATGGGAAGA
ATGATGGGATTACCCATAGGGCTTGTTTTAGGGAGAAACCTGTTCTCCAAACTCTTGGCCTTGAGATACCTGGTCCT
TATTCCTTGGACTTTGGCAATGTCTGACCCTCACATTCAAGTTCTGAGGAAGGGCCACTGCCTTCATACTGTGGATC
TGTAGCAAATTCCCCCTGAAAACCCAGAGCTGTATCTTAATTGTTTAAAAAAATTATATTATCTCAAGGACTGTTCT
TCTCTGAGTAGCCAAGCTCAGCTTGGTTCAAGCTACAAGCAGCTGAGCTGCTTTTTGTCTAGTCATTGTTCTTTTAT
TTCAGTGGATCAAATACGTTCTTTCCAAACCTAGGATCTTGTCTTCCTAGGCTATATATTTTGTCCCAGGAAGTCTT
AATCTGGGGTCCACAGAACACTAGGGGGCTGGTGAAGTTTATAGAAAAAAAATCTGTATTTTTACTTACATGTAACT
GAAATTTAGCATTTTCTTCTACTTTGAATGCAAAGGACAAACTAGAATGACATCATCAGTACCTATTGCATAGTTAT
AAAGAGAAACCACAGATATTTTCATACTACACCATAGGTATTGCAGATCTTTTTGTTTTTGTTTTTGTTTGAGATGG
AGTTTCGCTCTTATTGCCCAGGCTGGAGTGCAGTGGCATGATTTCGGCTCACTGCAACCTCCCCTTCCTGCATTCAA
GCAATTCTCCTGCCTTGGCCTCCAGAGTAGCTGGGGATTACAGGCACCTGCCACCATGCCAGTCTAATTTTTGTATT
TTTAGTAGAGATGGGGTTTCGCCATGTTGGCCAGGCTGGTCTTGAACTCCTGACCTCAGATGATCTGCCCGCCTTGG
CCTCCTGAAGTGCTGGGATTATAGGTGTGAGCCACCACGCCTGGCCCATTGCAGATATTTTTAATTCACATTTATCT
GCATCACTACTTGGATCTTAAGGTAGCTGTAGACCCAATCCTAGATCTAATGCTTTCATAAAGAAGCAAATATAATA
AATACTATACCACAAATGTAATGTTTGATGTCTGATAATGATATTTCAGTGTAATTAAACTTAGCACTCCTATGTAT
ATTATTTGATGCAATAAAAACATATTTTTTTTAGCACTTACAGTCTGCCAAACTGGCCTGTGACACAAAAAAAGTTT
AGGAATTCCTGGTTTTGTCTGTGTTAGCCAATGGTTAGAATATATGCTCAGAAAGATACCATTGGTTAATAGCTGAA
AGAAAATGGAGTAGAAATTCAGTGGCCTGGAATAATAACAATTTGGGCAGTCATTAAGTCAGGTGAAGACTTCTGGA
ATCATGGGAGAAAAGCAAGGGAGACATTCTTACTTGCCACAAGTGTTTTTTTTTTTTTTTTTTTATCACAAACATAA
GAAAATATAATAAATAACAAAGTCAGGTTATAGAAGAGAGAAACGCTCTTAGTAAACTTGGAATATGGAATCCCCAA
AGGCACTTGACTTGGGAGACAGGAGCCATACTGCTAAGTGAAAAAGACGAAGAACCTCTAGGGCCTGAACATAGGAA
ATTGTAGGAACAGAAATTCCTAGATCTGGTGGGGCAAGGGGAGCCATAGGAGAAAGAAATGGTAGAAATGGATGGAG
ACGGAGGCAGAGGTGGGCAGATCATGAGGTCAAGAGATCGAGACCATCCTGGCAAACATGGTGAAATCCCGTCTCTA
CTAAAAATAAAAAAATTAGCTGGGCATGGTGGCATGCGCCTGTAGTCCCAGCTGCTCGGGAGGCTGAGGCAGGAGAA
TCGTTTGAACCCAGGAGGCGAAGGTTGCAGTGAGCTGAGATAGTGCCATTGCACTCCAGTCTGGCAACAGAGTGAGA
CTCCGTCTCAAAAAAAAAAAAAAGAAAGAAAGAAAAGAAAAAGAAAAAAGAAAAAATAAATGGATGTAGAACAAGCC
AGAAGGAGGAACTGGGCTGGGGCAATGAGATTATGGTGATGTAAGGGACTTTTATAGAATTAACAATGCTGGAATTT
GTGGAACTCTGCTTCTATTATTCCCCCAATCATTACTTCTGTCACATTGATAGTTAAATAATTTCTGTGAATTTATT
CCTTGATTCTAAAATATGAGGATAATGACAATGGTATTATAAGGGCAGATTAAGTGATATAGCATAAGCAATATTCT
TCAGGCACATGGATCGAATTGAATACACTGTAAATCCCAACTTCCAGTTTCAGCTCTACCAAGTAAAGAGCTAGCAA
GTCATCAAAATGGGGACATACAGAAAAAAAAAAGGACACTAGAGGAATAATATACCCTGACTCCTAGCCTGATTAAT
ATATCGATTCACTTTTTTCTCTGTTTGATGACAAATTCTGGCTTTAAATAATTTTAGGATTTTAGGCTTCTCAGCTC
CCTTCCCAGTGAGAAGTATAAGCAGGACAGACAGGCAAGCAAGAAGAGAGCCCCAGGCAATACTCACAAAGTAGCCA
GTGTCCCCTGTGGTCATAGAGAAATGAAAAGAGAGAGGATTCCCTGGAAGCACTGGATGTAATCTTTTCTGTCTGTC
CTCTCTAGGGAATCACCCCAAGGTACTGTACTTTGGGATTAAGGCTTTAGTCCCACTGTGGACTACTTGCTATTCTG
TTCAGTTTCTAGAAGGAACTATGTACGGTTTTTGTCTCCCTAGAGAAACTAAGGTACAGAAGTTTTGTTTACAATGC
ACTCCTTAAGAGAGCTAGAACTGGGTGAGATTCTGTTTTAACAGCTTTATTTTCTTTTCCTTGGCCCTGTTTTTGTC
AACTGTCACCACCTTTAAGGCAAATGTTAAATGTGCTTTGGCTGAAACTTTTTTTCCTATTTTGAGATTTGCTCCTT
TATATGAGGCTTTCTTGGAAAAGGAGAATGGGAGAGATGGATATCATTTTGGAAGATGATGAAGAGGGTAAAAAAGG
GGACAAATGGAAATTTGTGTTGCAGATAGATGAGGAGCCAACAAAAAAGAGCCTCAGGATCCAGCACACATTATCAC
AAACTTAGTGTCCATCCATCACTGCTGACCCTCTCCGGACCTGACTCCACCCCTGAGGACACAGGTCAGCCTTGACC
AATGACTTTTAAGTACCATGGAGAACAGGGGGCCAGAACTTCGGCAGTAAAGAATAAAAGGCCAGACAGAGAGGCAG
CAGCACATATCTGCTTCCGACACAGCTGCAATCACTAGCAAGCTCTCAGGCCTGGCATCATGGTGCATTTTACTGCT
GAGGAGAAGGCTGCCGTCACTAGCCTGTGGAGCAAGATGAATGTGGAAGAGGCTGGAGGTGAAGCCTTGGGCAGGTA
AGCATTGGTTCTCAATGCATGGGAATGAAGGGTGAATATTACCCTAGCAAGTTGATTGGGAAAGTCCTCAAGATTTT
TTGCATCTCTAATTTTGTATCTGATATGGTGTCATTTCATAGACTCCTCGTTGTTTACCCCTGGACCCAGAGATTTT
TTGACAGCTTTGGAAACCTGTCGTCTCCCTCTGCCATCCTGGGCAACCCCAAGGTCAAGGCCCATGGCAAGAAGGTG
CTGACTTCCTTTGGAGATGCTATTAAAAACATGGACAACCTCAAGCCCGCCTTTGCTAAGCTGAGTGAGCTGCACTG
TGACAAGCTGCATGTGGATCCTGAGAACTTCAAGGTGAGTTCAGGTGCTGGTGATGTGATTTTTTGGCTTTATATTT
TGACATTAATTGAAGCTCATAATCTTATTGGAAAGACCAACAAAGATCTCAGAAATCATGGGTCGAGCTTGATGTTA
GAACAGCAGACTTCTAGTGAGCATAACCAAAACTTACATGATTCAGAACTAGTGACAGTAAAGGACTACTAACAGCC
TGAATTGGCTTAACTTTTCAGGAAATCTTGCCAGAACTTGATGTGTTTATCCCAGAGAATTGTATTATAGAATTGTA
GACTTGTGAAAGAAGAATGAAATTTGGCTTTTGGTAGATGAAAGTCCATTTCAAGGAAATAGAAATGCCTTATTTTA
TGTGGGTCATGATAATTGAGGTTTAGAAGAGATTTTTGCAAAAAAAATAAAAGATTTGCTCAAAGAAAAATAAGACA
CATTTTCTAAAATATGTTAAATTTCCCATCAGTATTGTGACCAAGTGAAGGCTTGTTTCCGAATTTGTTGGGGATTT
TAAACTCCCGCTGAGAACTCTTGCAGCACTCACATTCTACATTTACAAAAATTAGACAATTGCTTAAAGAAAAACAG
GGAGAGAGGGAACCCAATAATACTGGTAAAATGGGGAAGGGGGTGAGGGTGTAGGTAGGTAGAATGTTGAATGTAGG
GCTCATAGAATAAAATTGAACCTAAGCTCATCTGAATTTTTTGGGTGGGCACAAACCTTGGAACAGTTTGAGGTCAG
GGTTGTCTAGGAATGTAGGTATAAAGCCGTTTTTGTTTGTTTGTTTGTTTTTTCATCAAGTTGTTTTCGGAAACTTC
TACTCAACATGCCTGTGTGTTATTTTGTCTTTTGCCTAACAGCTCCTGGGTAACGTGATGGTGATTATTCTGGCTAC
TCACTTTGGCAAGGAGTTCACCCCTGAAGTGCAGGCTGCCTGGCAGAAGCTGGTGTCTGCTGTCGCCATTGCCCTGG
CCCATAAGTACCACTGAGTTCTCTTCCAGTTTGCAGGTCTTCCTGTGACCCTGACACCCTCCTTCTGCACATGGGGA
CTGGGCTTGGCCTTGAGAGAAAGCCTTCTGTTTAATAAAGTACATTTTCTTCAGTAATCAAAAATTGCAATTTTATC
TTCTCCATCTTTTACTCTTGTGTTAAAAGGAAAAAGTGTTCATGGGCTGAGGGATGGAGAGAAACATAGGAAGAACC
AAGAGCTTCCTTAAGAAATGTATGGGGGCTTGTAAAATTAATGTGGATGTTATGGGAGAATTCCCAAGATTCCCAAG
GAGGATGATATGATGGAGAAAAATCTTTATCGGGGTGGGAAAATGGTTAATTAAGTGGCAGAGACTCCTAGGCAGTT
TTTACTGCACCGGGGAAAGAAGGAGCTGTTGTGGTACCTGAGAAAGCAGATTTGTGGTACATGTCACTTTTCATTAA
AAACAAAAACAAAACAAAACAAAACTTCATAGATATCCAAGATATAGGCTGAGAATTACTATTTTAATTTACTCTTA
TTTACATTTTGAAGTAGCTAGCTTGTCACATGTTTTATGAAATTGATTTGGAGATAAGATGAGTGTGTATCAACAAT
AGCCTGCTCTTTCCATGAAGGATTCCATTATTTCATGGGTTAGCTGAAGCTAAGACACATGATATCATTGTGCATTA
TCTTCTGATACAATGTAACATGCACTAAAATAAAGTTAGAGTTAGGACCTGAGTGGGAAAGTTTTTGGAGAGTGTGA
TGAAGACTTTCCGTGGGAGATAGAATACTAATAAAGGCTTAAATTCTAAAACCAGCAAGCTAGGGCTTCGTGACTTG
CATGAAACTGGCTCTCTGGAAGTAGAAGGGAGAGTAAGACATACGTAGAGGACTAGGAAAGACCAGATAGTACAGGG
CCTGGCTACAAAAATACAAGCTTTTACTATGCTATTGCAATACTAAACGATAAGCATTAGGATGTTAAGTGACTCAG
GAAATAAGATTTTGGGAAAAAGTAATCTGCTTATGTGCACAAAATGGATTCAAGTTTGCAGATAAAATAAAATATGG
ATGATGATTCAAGGGGACAGATACAATGGTTCAAACCCAAGAGGAGCAGTGAGTCTGTGGAATTTTGAAGGATGGAC
AAAGGTGGGGTGAGAAAGACATAGTATTCGACCTGACTGTGGGAGATGAGAAGGAAGAAGGAGGTGATAAATGACTG
AAAGCTCCCAGACTGGTGAAGATAACAGGAGGAAACCATGCACTTGACCCTGGTGACTCTCATGTGTGAAGGGTAGA
GGGATATTAACAGATTTACTTTTTAGGAAGTGCTAGATTGGTCAGGGAGTTTTGACCTTCAGGTCTTGTGTCTTTCA
TATCAAGGAACCTTTGCATTTTCCAAGTTAGAGTGCCATATTTTGGCAAATATAACTTTATTAGTAATTTTATAGTG
CTCTCACATTGATCAGACTTTTTCCTGTGAATTACTTTTGAATTTGGCTGTATATATCCAGAATATGGGAGAGAGAC
AAATAATTATTGTAGTTGCAGGCTATCAACAATACTGGTCTCTCTGAGCCTTATAACCTTTCAATATGCCCCATAAA
CAGAGTAAACAGGGATTATTCATGGCACTAAATATTTTCACCTAGGTCAGTCAACAAATGGAGGCAATGTGCATTTT
TTGATACATATTTTTATATATTTATGGGGCATGTGATACTTACATGCCTAGAACATGTGACTGATTAAGTCTAGATA
TTTAGGATATCCATTACTTTGAGCATTTATCATTTCTATGTATTGAGAAAATTTCAAATCCTCATTTCTGACCATTT
TGAAATATATAATAAATAGTAATTAACTATAGTCACCCTACTCAAATATCAACATTATAAACTAACTAATCCTTCTT
TCCACTTTTTTACCAACCAACATCTCTTAAATCCCCTGCCATACACATCACACATTTTTCAGCTCTGATAACTATCA
TTCTACTCTCATACCACCATGAGACCACTTTTTTAGCTCCACAGATGAATAAAAACATGTGATATTTGACTTTCTGT
ATCTGGCTTATTTTATTATCTATCTCTTTGGCATACCAAGAGTTTGTTTTTGTTCTGCTTCAGGGCTTTCAATTAAC
ATAATGACCTCTGGTTCCATCCATGTTGCTACAAATGACAAGATTTCATTCTTTTTCATGGCAAAATAGTACTGTGC
AAAAAATACAATTTTTTAATCCGTTCATCTGTTGATAGACACTTAGGTTGATCCCAAACCTTAACTATTGTGAATAG
GTGCTTCAATAAACATGAGTGTAATGTGTCCATTGGATATACTGATTTCCTTTCTTTTGGATAAATAACCACTAGTG
AGATTGCTGGATTGTATGATAGTTCTGTTTTTAGTTTATTGAGAAATCTTCATACTGTTTTCCATAATGGTTGTACT
ATTTTACATTCCCACCAACAGTGTGTAAGAAAGAGTTCCCTTTTCTCCATATCCTCACAAGGATCTGTTATTTTTTG
TCTTTTTTGTTAATAGCATTTTAACTAGAGTAAGTAGATATCTCATTGTAGTTTTGATTTGCATTTCCCTGATCATT
AGTGATGTTGAGATTTTTTCATATGTTTGTTGGTCATTTGTATATCTTTTTCTGAGATTGTCTGTTCATGTCCTTAT
CCTACTTTTATTGGGATTGTTGTTATTTTCTTGATAATCATTGTGTCATTTTAGAGCCTGGATATTATTCTTTTGTC
AGATGTATAGATTGTGAAGATTTTCTCCTCTGTGGGTTGTCTGTTTATTCTGCAGACTCTTCCTTTTGCCATGCAAA
AGCTCTTTAGTTTAATTTAGTCCCAGATATTTTCTTTGTTTTTATGTGTTTGCATTTGTGTTCTTGTCATGAAATCC
TTTCCTAAGCCAATGTGTAGAAGGGTTTTTCCGATGTTATTTTCTAGAATTGTTACAGTTTCAGGCTTAGATTTAAG
TCCTTGATCCATCTTAAGTTGATTTTTGTATAAGGTGAGAGATGAAGATCCAGTTTCATTCTCCTACATGTAGCTTG
CCAGCTATCCCGACTCATTTGTTGAATAGGGTGCCCTTTCCCATTTATGTTTTTGTTTGCTTTGTCAAAGATCAGTT
CGGATGTAAGTATTTGAGTTTATTTCTGGGTTCTCTATTCTGTTCCATTGGTCCGATGTGCCTATTTGTACACCAGC
ATCATGCTGTGTTTTTGGTGACTATGGCCTTATTGTATAGTTTGAAATGAGGTAATGTAATGCCATTCAGATTTGTT
CTTTTTTTTAGACTTGCTTGTTTATTGGGCTCTTTTTTGGTTCCATAAGAATTTTAGGATTGTTTTTTCTAGTTCTG
TGAAGGCTAATGGTGGTATTTATGGGAATTGCAATGCAATTTGTAGGTTGCTTCTGGCATTATGGCCATTTTCACAA
TATTGATTCTACCCATCTATGAGAATGGCATGTGTTTCCATTTGTTTGTGTCTTATATGATTACTATCAGCCGTGTT
TTGTAGTTTTCCTTGTAGATGTCTTTCACCTCCTTGGTTAGGTATATATTCCTAAGTTTTTGTTTTGTTTTGTTTTG
TTTTTTGCAGCTATTGTAAAAGGGGTTGAGTTATTGATTTTATTCTCATCTTGGTCATTGCTGGTATGTAAGAAAGC
AACTCATTGGTGTACGTTAATTTTGTATCCAGAAACTTTGCTGAATTATTTTATCAGTTCTAGGGGGTTTTGGAGGA
GTCTTTAGAGTTTTCTACATACACAATCATATCATCAGCAAACAGTGACAGTTTGACTTTCTCTTTAACAATTTGGA
TGTGCTTTACTTGTTTCTCTTGTCTGATTGCTCTTGCTAGGACTTCCAGTAATATGTTAAAGAGAAGTGGTGAGAGT
GGGTATCCTTGTCTCATTCCAGTTTTCAGACAGAATGCTTTTAACTTTTTCCCATTCAATATAATGTTGGCTGTGTG
TTTACCATAGCTGGCTTTTATTACATTGAGGTATGTCCTTTGTAAACCGATTTTGCTGAGTTTTAGTCATAAAGTGA
TGTTGAATTTTGTTGAATGCAGTTTCTGTGGCTATTGAGATAATCACATGATTTTTGTTTCCAATTCTCTTTATGTT
GTGTATCACACTTATTGACTTGCGTATGTTAAACCATCCGTGCATCCCTCGCATGAAACCACTTGATCATGGGTTTT
GATATGCCGTGTGGGATGCTATTAGCTATATTTTGTCAAGGATGTTGGCATCTATGTTCATCAGGGATATTGATCTG
TAGTGTTTTTTTTTTTTGGTTATGTTCTTTCCCAGTTTTGGTATTAAGGTGATACTGGCTTCATAGAATGATTTAGG
GAGGATTCTCTCTTTCTCTATCTTGTAGAATACTGTCAATAGGATTGGTATCAATTCTTCTTTGAATGTCTGGTAGA
ATTCGAACGTCTCCTTTAGGTTTTCTAGTTTATTCATGTAAAGGTGTTCATAGTAACCTTGAATAATCTTTTGTATT
TCTGTGGTATCAGTAATAGTATCTCCTGTTTTGTTTCTAACTGAGTTTATTTGCACTTCTCTCCTCTTTTCTTGGTT
AATCTTGCTAATGGTCTATCAGTTTTATTTATCTTTTCAAAGAACCAGCTTTTTATTTCATTTAGCTTTTGTATTTT
TTTGCAGTTGTTTTAATTTCATTTAGTTCTCCTCTTATCTTAGTTATTCCCTTTCTTTTGCTGGGTTTTGGTTCTGT
TTGTTTTTGTTTCTCTAGTTTCTTGTGGTGTGACCTTATATTGTCTGTCCTCTTTCAGACTCTTTGACATCGACATT
TAGGGCTGTGAACTTTCCTTTTAGCACCATCTTTGCTGTATCCTAGAGGTTTTGATAGGTGTGTCACTATTGTCGGT
CAGTTCAAGTAATTTTGTTGTTCTTATTATACTTTAAGTTCTGGGATACATGTGCAGAATGTGCAGGTTTGTTACAT
AGGTATAGATGTGCCATGGTGGTTTGCTGCTCCCATCAACCTGTCATCTACATTAGGTATTTCTTTTAATGTTATCC
CTCTCCTAACCCCCTCACCCCCCGACAGGCCCTGGTGTGTGATGTTCCCCTCCCTGTGTCCATGTGTTCTCATTGTT
CAACTCCCACTTATGAGTGAGAACGTGTGGTGTTTGGTTTCTCTGTTCCTGTGTTAGTTTGCTCAGAATGATGTTTC
CACCTTCACCATGTCCCTGCAAAGACATGAACTCATCATTTTATGGCTGCATATATTCCATGGTGTATATGTGCCAC
ATTTTCTTTATCCATTATATCGCTGATGGCCATTTGGGTTGGTTCCAAGTCTTTGGTATTGTGAATAGTGCCGCAAT
AAACATACGTGTGCACATGTCTTTATAGTAGAATGATTTCTAATTCTTTGGGTATATACCCAGTAATGGGATTGCTG
GGTCAAACAGTATTTCTGGTTCTAGATCCTTGAGGAATTGCCACACTGTCTTCCACAATGGTTGAACTAATTTACAC
ACCCATCAACAGTGTAAAATTTTTCCTATTCTTCCACATCCTCTCCAGCACCTTTTGTTTCCTGACTTTTTAATAAT
TGCCATTCTAACTGGCATGAGATGGTATCTCATTGTGGTTTTGATTTGCATTTCTCTAATGACCAGTGATGATGAGC
TTCTTTTCATGTGTTTCTTGGCCACATAAATGACTTCTTTAGAGAAGCATCTGTTCATATCCTTTGTCCACTTTTTG
ATGGGGTCGTTAGGTTTTTTCTTGTAAATTTGTTGAAGTTCTTTGTAGATTTTGGATGTTAGCCCTTTGTCAGATGG
ATAGATTGGCAAAAATTTTCTCCCATTCTGTAGGTTGCCTGTTCACTCTGATGATAGTCTTTTGCTGTGCAGAAGCT
CTTTAGTTTAATTAGATCCCATATGTCAATTTTGGCCTTTGTTGTCATTGCTTTTGATGTTTAGTCGTGGAATTTTG
CCCATGCCTATGTCCTGAATGGTATTGCCTAGGTTATCTTCTAGGATTTTTATGGTTTTAGGTTGCACATTTAAGTC
TTTAATCCACCTTGAGTTAATTTTTGTATAAGGTGTAAGGAAGGGGTACAGTTTCAGTTTTATGCATATTGCTAGCC
AGTTTTTCCAGCACCATTTATTAAATAGGGAATTCTTTCTCCATTGCTTTTGTGATGTTTGTCAAAGATCAGATGGT
CGTAGATGTGTGGCATTATTTCTGAGGCTTCTGTTCTGTTCCACTGGTCTATATATCTGTTTTGGTACCAGTACCAT
GCTGTTTTTGTTACTGTAGCCTTGTAGTATAGCTTGAAGTCAGGTAGCATCATGCCTCCAGCTTTGTTCTTTTTGTT
TAGGATTGTCTTGGCTATATGGGCTCTTTTTTGATTCCATATGACATTTAAAGTAGTTTTTTCTAATTCTTTGAAAA
AAGTCAGTGGTAGCTTGATGGGGATAGCATTGAATCTATAAATTACTTTGGGCAGTATGGCCATTTTAAAGATATTG
ATTCTTTCTATCTATGAGCATGGAATGTTTTTCCATTTGTTTGTGTCCTCTCTTATTTCCTTGAGCAGTGAGTGGTT
TGTAGCTCTCCTTGAAGAGGTTCTTCACATCCCTTATAAGTTGTATTTCTAGGTATTTTATTTTATTCTCTTTGCAG
CAATTGTGAATGGGAGTTCACCCATGATTTGGCTCTCTGCTTGTCTATTATTGGTGTATAGGAATGCTTGTGATTTT
TGCACACTGATTTTGTATCTTGAGACTTTGCTGAAGCTGTTTATCAGCTTAAGATTTTGGGCTGAGATGACAGGGTC
TTCTAAATATACAATCATGTCATCTGCAAACAGAGACAATTTGACTTCCTCTCTTCCTATTTGAATATGCTTTATTT
CTTTCTCTTGCCTGATTGTCCTGGCGAGAACTTCCAATACTATGTTGAGTAAGAGTGGCGAGAGGGCATCCTTGTCT
TGTGCCGGTTTTCAAAGCAAATGATTTTTAAATTTCCGTCTTGATTTCATTGTTGACCCAATGATCATTCAGGAGCA
GGTTATTTAATTTCCCTGTATTTGCATGGTTTTGAAGGTTCCTTTTGTAGTTGATTTCCAATTTTATTCTACTGTGG
TCTGAGAGAGTGCTTGATATAATTTCAATTTTTAAAAATTTATTGAGGCTTGTTTTGTGGCATATCATATGGCCTAT
CTTGGAGAAAGTTCCATGTGCTGATGAATAGAATGTGTATTCTGCAGTTGTTGGGTAGAATGTCCTGTAAATATCTG
TTAAGTCCATTTGTTCTTTAAATCCATTGTTTCTTTGTAGACTGTCTTGATGACCTGCCTAGTGCAGTCAGTGGAGT
ATTGAAGTCCCCCACTATTATTATGTTGCTGTCTAGTAGTAATTGTTTTATAAATTTGGGATCTCCAGTATTAGATG
CATATATATTAAGAATTGTAATATTCTCCCATTGGACAAGGGCTTTTATCATTATATGATGTCCCTCTTTGTCTTTT
TTAACTGCTGTTTCTTTAAAGTTTGTTTTGTCTGACATAAGAATAGCTGCTTTGGCTCGCTTTTGGTGTCCATTTGT
GTGGAATGTCATTTTCCACCCCTTTACCTTAAGTTTATGTGAGTCCTTATGTGTTAGGTGAGTCTCCTGAAGGCGGC
AGATAACTGGTTGGTGAATTCTATTCATTCTGCAATTCTGTATCTTTTAAGTGGAGCATTTAGTCCATTTACATTCA
ACATCAGTATTGAGGTGTGAGGTGACTATTCCATTCTTCGTGGTATTTGTTGCCTGTGTATCTTTTTATCTGTATTT
TTGTTGTATATGTCCTATGGGATTTATGCTTTAAAGAGGTTCTGTTTTGATGTGCTTCCAGGGTTTATTTCAAGATT
TAGAGCTCCTTTTATCATTCTTGTAGTGTTGGCTTGGTAGTGCCGAATTCTCTCAGCATTTGTTTTTCTGAAAAACA
CTGTGTATTTTCTTCATTTGTGAAGCTTAGTTTCACTGGATATAAAATTCTTGGCTGATAATTGTTTTGTTTAAGAA
GGCTGAAGATAGGGCCATATTCACTTCTAGCTTTTACGGTTTCTGCTGAGAAATCTGCTGTTAATCTGATAGGTTTT
CTTTCATAGGTTACCTGGTAGTTTCACCTCACAGCTCTTAAGATTCTCTTTGTCTTTAGATAACTTTGGATACTCTG
ATGACAATGTACCTAGGCAATGATATTTTTGCAATGAATTTCCCAGGTGTTTATTGAGCTTCTTTGTATTTGGATAT
CTAGGTCTCTAGCAAGGAGGGGGAAGTTTTCCTTGATTATTTCCATGGACAAGTTTTCCAAACTTTTAGATTTCTCT
TCTTTCTCAGGAATGCTGATTATTCTTAGGTTTGATTGTTTAACATAATCCCAGATTTCTTGGAGGCTTTGTTCATA
TTTTCTTATTCTTTTTTCTTTGTCTTTGTTGGATTGGGTAATTCAAAAACTTTGTCTTCAAGCTCTGAATTTCTTCT
GCTTGGATTCTATTGCTGAGACTTTCTAGAGCATTTTGCATTTCTATAAGTGCATCCATTCATCCATTGTTTCCTGA
AGTTTTGAATGTTTTTTATTTATGCTATCTCTTTAACTGAAGATTTCTCCCCTCATTTCTTGTATCATATTTTTGGT
TTTTTTAAAATTGGACTTCACCTTCCTCGGATGCCTCCTTGATTAGCTTAATAACTGACCTTCTGAATTATTTTTCA
GGTAAATCAGGGATTTCTTCTTGGTTTGGATGCATTGCTGGTGAGCTAGTATGATTTTTTGGGGGGTGTTAAAGAAC
CTTGTTTTTCATATTACCAGAGTTAGTTTTCTGGTTCCTTCTCACTTGGGTAGGCTCTGTCAGAGGGAAAGTCTAGG
CCTCAAGGCTGAGACTTTTGTCCCAGCAGGTGTTCCCTTGATGTAGCACAGTCCCCCTTTTCCTAGGACGTGGGGCT
TCCTGAGAGCCGAACTGTAGTGATTGTTATCTCTCTTCTGGATCTAGCCACCCATCAGGTCTACCAGACTCCAGGCT
GGTACTGGGGTTTGTCTGCACAGAGTCTTGTGACGTGAACCATCTGTGGGTCTCTCAGCCATAGATACAACCACCTG
CTCCAATGGAGGTGGTAGAGGATGAAATGAACTCTGTGAGGGTCCTTACTTTTGGTTGTTCAATGCACTATCTTTTT
GTGCTGGTTGGCCTCCTGCCAGGAGGTGGCACTTTCTAGAAAGCATCAGCAGAGGCAGTCAGGTGGTGGTGGCTGGG
GGGGCTGGGGCACTAGAACTCCCAAGAATATATGCCCTTTGTCTTCAGCTACTAGGGTGAGTAAGGAAGGACCATCA
GGTGGGGGCAGGACTAGTCGTGTCTGAGCTCAGAGTCTCCTTGGGCAGGTCTTTCTGTGGCTACTGTGGGAGGATGG
GGGTGTAGTTTCCAGGTCAATGGATTTATGTTCCTAGGACAATTATGGCTGCCTCTGCTGTGTCATGCAGGTCATCA
GGAAAGTGGGGGAAAGCAAGCAGTCACGTGACTTGCCCAGCTCCCATGCAACTCAAAAGGTTGGTCTCACTTCCAGC
GTGCACCCTCCCCCGCAACAGCTCCGAATCTGTTTCCATGCAGTCAGTGAGCAAGGCTGAGAACTTGCCCAGGCTAC
CAGCTGCGAAACCAAGTAGGGCTGTCCTACTTCCCTGCCAGTGGAGTCTGCACACCAAATTCATGTCCCCCCACCAA
CCCCCCCACTGCCCAGCCCCTAGATCTGGCCAGGTGGAGATTTTCTTTTTCCTGTCTCTTTTCCCAGTTCCTCTGGC
AGCCCTCCCAAATGACCCCTGTGAGGCAAGGCAGAAATGGCTTCCTAGGGGACCCAGAGAGCCCACAGGGCTTTTCC
CGCTGCTTCCTCTACCCCTGTATTTTGCTTGGCCCTCTAAATTGACTCAGCTCCAGGTAAGGTCAGAATCTTCTCCT
GTGGTCTAGATCTTCAGGTTCCCAGTGAGGATGTGTGTTTGGGGGTAGACGGTCCCCCTTTTCCACTTCCACAGTTT
GGGCACTCACAATATTTGGGGTGTTTCCCGGGTCCTACATGAGCAATCTGCTTCTTTCAGAGGGTGTGTGCGTTCTC
TCAGCTTTCTTGAATTTATTTCTGCAGGTGGTTCTGCAAAAAAAATTCCTGATGGGAGACTTCACATGCTGCTCTGT
GCATCCGAGTGGGAGCTGCAATGTACTTCTGCTGCCACCCATCTGCCATCACCCTCTAATTTGTCGGTAATATGCAT
TTTTAATCAATCTTTTTTTCTCTCTCTCTCTTTTCTTCTCCCCCAAAACTATACTGCCCTTTGATATCAAGGAATCA
AGGCCGTGATGTTGAGGGGTGGGCAGTGGATACACTCTTTACCCCTTAGGGAGCATATCTAGATTTAGATATTGCCA
ATTCAAGATAACTTAATTGAAAGCAAATTCATAATGAATACACACACACACACACACATCTGCATGACAAGATTTTT
AATAGTTGAAAGAATAACTAATAATTGTCCACAGGCAATAAGGGCTTTTTAAGCAAAACAGTTGTGATAAAACAGGT
CATTCTTAGAATAGTAATCCAGCCAATAGTACAGGTTGCTTAGAGATTATGACATTACCAGAGTTAAAATTCAATAA
TGGCTTCTCACTCCCTACCACTGAGGACAAGTTTATGTCCTTAGGTTTATGCTTCCCTGAAACAATACCACCTGCTA
TTCTCCACTTTACATATCAACGGCACTGGTTCTTTATCTAACTCTCTGGCACAGCAGGAGTTTGTTTTCTTCTGCTT
CAGAGCTTTGAATTTACTATTTCAGCTTCTAAACTTTATTTGCAATGCCTTCCCATGGCAGACTCCTTCTGTCATTT
TGCCTCTGTTCGAAAACTTTTTCCTTAATTTCATTCTTAGTTAATAATATCTGAAATTATTTTGTTGTTTAACTTAA
TTATTAATTTTATGTATGTTCTACCTAGATATAATCTTCTAGAGGATTGTTTTATTCTCTGACTTATTTAACTTAAA
TGCCCACTACCTTTAAAAATTATGACATTTATTTAACAGATATTTGCTGAACAAATGTTTGAAAATACATGGGAAAG
AATGCTTGAAAACACTTGAAATTGCTTGTGTAAAGAAACAGTTTTATCAGTTAGGATTTAATCAATGTCAGAAGCAA
TGATATAGGAAAAATCGAGGAATAAGACAGTTATGGATAAGGAGAAATCAACAAACTCTTAAAAGATATTGCCTCAA
AAGCATAAGAGGAAATAAGGGTTTATACATGACTTTTAGAACACTGCCTGGGTTTTTGGATAAATGGGGAAGTTGTT
GGAAAACAGGAGGGATCCTAGATATTCCTTAGTCTGAGGAGGAGCAATTAAGATTCACTTGTTTAGAGGCTGGGAGT
GGTGGCTCACGCCTGTAATCCCAGAATTTTGGGAGGCCAAGGCAGGCAGATCACCTGAGGTCAAGAGTTCAAGACCA
ACCTGGCCAACATGGTGAAATCCCATCTCTACAAAAATACAAAAATTAGACAGGCATGATGGCAAGTGCCTGTAATC
CCAGCTACTTGGGAGGCTGAGGAAGGAGAATTGCTTGAACCTGGAAGGCAGGAGTTGCAGTGAGCCGAGATCATACC
ACTGCACTCCAGCCTGGGTGACAGAACAAGACTCTGTCTCAAAAAAAAAAAAGAGAGATTCAAAAGATTCACTTGTT
TAGGCCTTAGCGGGCTTAGACACCAGTCTCTGACACATTCTTAAAGGTCAGGCTCTACAAATGGAACCCAACCAGAC
TCTCAGATATGGCCAAAGATCTATACACACCCATCTCACAGATCCCCTATCTTAAAGAGACCCTAATTTGGGTTCAC
CTCAGTCTCTATAATCTGTACCAGCATACCAATAAAAATCTTTCTCACCCATCCTTAGATTGAGAGAAGTCACTTAT
TATTATGTGAGTAACTGGAAGATACTGATAAGTTGACAAATCTTTTTCTTTCCTTTCTTATTCAACTTTTATTTTAA
CTTCCAAAGAACAAGTGCAATATGTGCAGCTTTGTTGCGCAGGTCAACATGTATCTTTCTGGTCTTTTAGCCGCCTA
ACACTTTGAGCAGATATAAGCCTTACACAGGATTATGAAGTCTGAAAGGATTCCACCAATATTATTATAATTCCTAT
CAACCTGATAAGTTAGGGGAAGGTAGAGCTCTCCTCCAATAAGCCAGATTTCCAGAGTTTCTGACGTCATAATCTAC
CAAGGTCATGGATCGAGTTCAGAGAAAAAACAAAAGCAAAACCAAACCTACCAAAAAATAAAAATCCCAAAGAAAAA
ATAAAGAAAAAAACAGCATGAATACTTCCTGCCATGTTAAGTGGCCAATATGTCAGAAACAGCACTGAGTTACAGAT
AAAGATGTCTAAACTACAGTGACATCCCAGCTGTCACAGTGTGTGGACTATTAGTCAATAAAACAGTCCCTGCCTCT
TAAGAGTTGTTTTCCATGCAAATACATGTCTTATGTCTTAGAATAAGATTCCCTAAGAAGTGAACCTAGCATTTATA
CAAGATAATTAATTCTAATCCATAGTATCTGGTAAAGAGCATTCTACCATCATCTTTACCGAGCATAGAAGAGCTAC
ACCAAAACCCTGGGTCATCAGCCAGCACATACACTTATCCAGTGATAAATACACATCATCGGGTGCCTACATACATA
CCTGAATATAAAAAAAATACTTTTGCTGAGATGAAACAGGCGTGATTTATTTCAAATAGGTACGGATAAGTAGATAT
TGAAGTAAGGATTCAGTCTTATATTATATTACATAACATTAATCTATTCCTGCACTGAAACTGTTGCTTTATAGGAT
TTTTCACTACACTAATGAGAACTTAAGAGATAATGGCCTAAAACCACAGAGAGTATATTCAAGAATAAGTATAGCAC
TTCTTATTTGGAAACCAATGCTTACTAAATGAGACTAAGACGTGTCCCATCAAAAATCCTGGACCTATGCCTAAAAC
ACATTTCACAATCCCTGAACTTTTCAAAAATTGGTACATGCTTTAACTTTAAACTACAGGCCTCACTGGAGCTACAG
ACAAGAAGGTGAAAAACGGCTGACAAAAGAAGTCCTGGTATCTTCTATGGTGGGAGAAGAAAACTAGCTAAAGGGAA
GAATAAATTAGAGAAAAATTGGAATGACTGAATCGGAACAAGGCAAAGGCTATAAAAAAAATTAAGCAGCAGTATCC
TCTTGGGGGCCCCTTCCCCACACTATCTCAATGCAAATATCTGTCTGAAACGGTTCCTGGCTAAACTCCACCCATGG
GTTGGCCAGCCTTGCCTTGACCAATAGCCTTGACAAGGCAAACTTGACCAATAGTCTTAGAGTATCCAGTGAGGCCA
GGGGCCGGCGGCTGGCTAGGGATGAAGAATAAAAGGAAGCACCCTTCAGCAGTTCCACACACTCGCTTCTGGAACGT
CTGAGGTTATCAATAAGCTCCTAGTCCAGACGCCATGGGTCATTTCACAGAGGAGGACAAGGCTACTATCACAAGCC
TGTGGGGCAAGGTGAATGTGGAAGATGCTGGAGGAGAAACCCTGGGAAGGTAGGCTCTGGTGACCAGGACAAGGGAG
GGAAGGAAGGACCCTGTGCCTGGCAAAAGTCCAGGTCGCTTCTCAGGATTTGTGGCACCTTCTGACTGTCAAACTGT
TCTTGTCAATCTCACAGGCTCCTGGTTGTCTACCCATGGACCCAGAGGTTCTTTGACAGCTTTGGCAACCTGTCCTC
TGCCTCTGCCATCATGGGCAACCCCAAAGTCAAGGCACATGGCAAGAAGGTGCTGACTTCCTTGGGAGATGCCATAA
AGCACCTGGATGATCTCAAGGGCACCTTTGCCCAGCTGAGTGAACTGCACTGTGACAAGCTGCATGTGGATCCTGAG
AACTTCAAGGTGAGTCCAGGAGATGTTTCAGCACTGTTGCCTTTAGTCTCGAGGCAACTTAGACAACTGAGTATTGA
TCTGAGCACAGCAGGGTGTGAGCTGTTTGAAGATACTGGGGTTGGGAGTGAAGAAACTGCAGAGGACTAACTGGGCT
GAGACCCAGTGGCAATGTTTTAGGGCCTAAGGAGTGCCTCTGAAAATCTAGATGGACAACTTTGACTTTGAGAAAAG
AGAGGTGGAAATGAGGAAAATGACTTTTCTTTATTAGATTTCGGTAGAAAGAACTTTCACCTTTCCCCTATTTTTGT
TATTCGTTTTAAAACATCTATCTGGAGGCAGGACAAGTATGGTCGTTAAAAAGATGCAGGCAGAAGGCATATATTGG
CTCAGTCAAAGTGGGGAACTTTGGTGGCCAAACATACATTGCTAAGGCTATTCCTATATCAGCTGGACACATATAAA
ATGCTGCTAATGCTTCATTACAAACTTATATCCTTTAATTCCAGATGGGGGCAAAGTATGTCCAGGGGTGAGGAACA
ATTGAAACATTTGGGCTGGAGTAGATTTTGAAAGTCAGCTCTGTGTGTGTGTGTGTGTGTGTGCGCGCGTGTGTTTG
TGTGTGTGTGAGAGCGTGTGTTTCTTTTAACGTTTTCAGCCTACAGCATACAGGGTTCATGGTGGCAAGAAGATAAC
AAGATTTAAATTATGGCCAGTGACTAGTGCTGCAAGAAGAACAACTACCTGCATTTAATGGGAAAGCAAAATCTCAG
GCTTTGAGGGAAGTTAACATAGGCTTGATTCTGGGTGGAAGCTTGGTGTGTAGTTATCTGGAGGCCAGGCTGGAGCT
CTCAGCTCACTATGGGTTCATCTTTATTGTCTCCTTTCATCTCAACAGCTCCTGGGAAATGTGCTGGTGACCGTTTT
GGCAATCCATTTCGGCAAAGAATTCACCCCTGAGGTGCAGGCTTCCTGGCAGAAGATGGTGACTGGAGTGGCCAGTG
CCCTGTCCTCCAGATACCACTGAGCTCACTGCCCATGATGCAGAGCTTTCAAGGATAGGCTTTATTCTGCAAGCAAT
ACAAATAATAAATCTATTCTGCTAAGAGATCACACATGGTTGTCTTCAGTTCTTTTTTTTATGTCTTTTTAAATATA
TGAGCCACAAAGGGTTTTATGTTGAGGGATGTGTTTATGTGTATTTATACATGGCTATGTGTGTTTGTGTCATGTGC
ACACTCCACACTTTTTTGTTTACGTTAGATGTGGGTTTTGATGAGCAAATAAAAGAACTAGGCAATAAAGAAACTTA
TACATGGGAGCGTCTGCAAGTGGGAGTAAAAGGTGCAGGAGAAATCTGGTTGGAAGAAAGACCTCTATAGGACAGGA
CTCCTCAGAAACAGATGTTTTGGAAGAGATGGGGAAAGGTTCAGTGAAGGGGGCTGAACCCCCTTCCCTGGATTGCA
GCACAGCAGCGAGGAAGGGGCTCAACGAAGAAAAAGTGTTCCAAGCTTTAGGAAGTCAAGGTTTAGGCAGGGATAGC
CATTCTATTTTATTAGGGGCAATACTATTTCCAACGGCATCTGGCTTTTCTCAGCCCTTGTGAGGCTCTACGGGGAG
GTTGAGGTGTTAGAGATCAGAGCAGGAAACAGGTTTTTCTTTCCACGGTAACTACAATGAAGTGATCCTTACTTTAC
TAAGGAACTTTTTCATTTTAAGTGTTGACGCATGCCTAAAGAGGTGAAATTAATCCCATACCCTTAAGTCTACAGAC
TGGTCACAGCATTTCAAGGAGGAGACCTCATTGTAAGCTTCTAGGGAGGTGGGGACCTAGGTGAAGGAAATGAGCCA
GCAGAAGCTCACAAGTCAGCATCAGCGTGTCATGTCTCAGCAGCAGAACAGCACGGTCAGATGAAAATATAGTGTGA
AGAATTTGTATAACATTAATTGAGAAGGCAGATTCACTGGAGTTCTTATATAATTGAAAGTTAATGCACGTTAATAA
GCAAGAGTTTAGTTTAATGTGATGGTGTTATGAACTTAACGCTTGTGTCTCCAGAAAATTCACATGCTGAATCCCCA
ACTCCCAATTGGCTCCATTTGTGGGGGAGGCTTTGGAAAAGTAATCAGGTTTAGAGGAGCTCATGAGAGCAGATCCC
CATCATAGAATTATTTTCCTCATCAGAAGCAGAGAGATTAGCCATTTCTCTTCCTTCTGGTGAGGACACAGTGGGAA
GTCAGCCACCTGCAACCCAGGAAGAGAGCCCTGACCAGGAACCAGCAGAAAAGTGAGAAAAAATCCTGTTGTTGAAG
TCACCCAGTCTATGCTATTTTGTTATAGCACCTTGCACTAAGTAAGGCAGATGAAGAAAGAGAAAAAAATAAGCTTC
GGTGTTCAGTGGATTAGAAACCATGTTTATCTCAGGTTTACAAATCTCCACTTGTCCTCTGTGTTTCAGAATAAAAT
ACCAACTCTACTACTCTCATCTGTAAGATGCAAATAGTAAGCCTGATCCCTTCTGTCTAACTTCGAATTCTATTTTT
TCTTCAACGTACTTTAGGCTTGTAATGTGTTTATATACAGTGAAATGTCAAGTTCTTTCTTTATATTTCTTTCTTTC
TTTTTTTTCCTCAGCCTCAGAGTTTTCCACATGCCCTTCCTACCTTCAGGAACTTCTTTCTCCAAACGTCTTCTGCC
TGGCCTCCATTCAAATCATAAAGGACCCACTTCAAATGCCATCACTCACTACCATTTCACAATTCGCACTTTCTTTC
TTTGTCCTTTTTTTTTTTAGTAAAACAAGTTTATAAAAAATTGAAGGAATAAATGAATGGCTACTTCATAGGCAGAG
TAGACACAAGGGCTACTGGTTGCCGATTTTTATTGTTATTTTTCAATAGTATGCTAAACAAGGGGTAGATTATTTAT
GCTGCCCATTTTTAGACCATAAAAGATAACTTCCTGATGTTGCCATGGCATTTTTTTTCCTTTTAATTTTATTTCAT
TTCATTTTAATTTCGAAGGTACATGTGCAGGATGTGCAGGCTTGTTACATGGGTAAATGTGTGTCTTTCTGGCCTTT
TAGCCATCTGTATCAATGAGCAGATATAAGCTTTACACAGGATCATGAAGGATGAAAGAATTTCACCAATATTATAA
TAATTTCAATCAACCTGATAGCTTAGGGGATAAACTAATTTGAAGATACAGCTTGCCTCCGATAAGCCAGAATTCCA
GAGCTTCTGGCATTATAATCTAGCAAGGTTAGAGATCATGGATCACTTTCAGAGAAAAACAAAAACAAACTAACCAA
AAGCAAAACAGAACCAAAAAACCTCCATAAATACTTCCTACCCAGTTAATGGTCCAATATGTCAGAAACAGCACTGT
GTTAGAAATAAAGCTGTCTAAAGTACACTAATATTCGAGTTATAATAGTGTGTGGACTATTAGTCAATAAAAACAAC
CCTTGCCTCTTTAGAGTTGTTTTCCATGTACACGCACATCTTATGTCTTAGAGTAAGATTCCCTGAGAAGTGAACCT
AGCATTTATACAAGATAATTAATTCTAATCCACAGTACCTGCCAAAGAACATTCTACCATCATCTTTACTGAGCATA
GAAGAGCTACGCCAAAACCCTGGGTCATCAGCCAGCACACACACTTATCCAGTGGTAAATACACATCATCTGGTGTA
TACATACATACCTGAATATGGAATCAAATATTTTTCTAAGATGAAACAGTCATGATTTATTTCAAATAGGTACGGAT
AAGTAGATATTGAGGTAAGCATTAGGTCTTATATTATGTAACACTAATCTATTACTGCGCTGAAACTGTGGTCTTTA
TGAAAATTGTTTTCACTACACTATTGAGAAATTAAGAGATAATGGCAAAAGTCACAAAGAGTATATTCAAAAAGAAG
TATAGCACTTTTTCCTTAGAAACCACTGCTAACTGAAAGAGACTAAGATTTGTCCCGTCAAAAATCCTGGACCTATG
CCTAAAACACATTTCACAATCCCTGAACTTTTCAAAAATTGGTACATGCTTTAGCTTTAAACTACAGGCCTCACTGG
AGCTACAGACAAGAAGGTAAAAAACGGCTGACAAAAGAAGTCCTGGTATCCTCTATGATGGGAGAAGGAAACTAGCT
AAAGGGAAGAATAAATTAGAGAAAAACTGGAATGACTGAATCGGAACAAGGCAAAGGCTATAAAAAAAATTAAGCAG
CAGTATCCTCTTGGGGGCCCCTTCCCCACACTATCTCAATGCAAATATCTGTCTGAAACGGTCCCTGGCTAAACTCC
ACCCATGGGTTGGCCAGCCTTGCCTTGACCAATAGCCTTGACAAGGCAAACTTGACCAATAGTCTTAGAGTATCCAG
TGAGGCCAGGGGCCGGCGGCTGGCTAGGGATGAAGAATAAAAGGAAGCACCCTTCAGCAGTTCCACACACTCGCTTC
TGGAACGTCTGAGATTATCAATAAGCTCCTAGTCCAGACGCCATGGGTCATTTCACAGAGGAGGACAAGGCTACTAT
CACAAGCCTGTGGGGCAAGGTGAATGTGGAAGATGCTGGAGGAGAAACCCTGGGAAGGTAGGCTCTGGTGACCAGGA
CAAGGGAGGGAAGGAAGGACCCTGTGCCTGGCAAAAGTCCAGGTCGCTTCTCAGGATTTGTGGCACCTTCTGACTGT
CAAACTGTTCTTGTCAATCTCACAGGCTCCTGGTTGTCTACCCATGGACCCAGAGGTTCTTTGACAGCTTTGGCAAC
CTGTCCTCTGCCTCTGCCATCATGGGCAACCCCAAAGTCAAGGCACATGGCAAGAAGGTGCTGACTTCCTTGGGAGA
TGCCATAAAGCACCTGGATGATCTCAAGGGCACCTTTGCCCAGCTGAGTGAACTGCACTGTGACAAGCTGCATGTGG
ATCCTGAGAACTTCAAGGTGAGTCCAGGAGATGTTTCAGCACTGTTGCCTTTAGTCTCGAGGCAACTTAGACAACTG
AGTATTGATCTGAGCACAGCAGGGTGTGAGCTGTTTGAAGATACTGGGGTTGGGAGTGAAGAAACTGCAGAGGACTA
ACTGGGCTGAGACCCAGTGGCAATGTTTTAGGGCCTAAGGAGTGCCTCTGAAAATCTAGATGGACAACTTTGACTTT
GAGAAAAGAGAGGTGGAAATGAGGAAAATGACTTTTCTTTATTAGATTTCGGTAGAAAGAACTTTCACCTTTCCCCT
ATTTTTGTTATTCGTTTTAAAACATCTATCTGGAGGCAGGACAAGTATGGTCGTTAAAAAGATGCAGGCAGAAGGCA
TATATTGGCTCAGTCAAAGTGGGGAACTTTGGTGGCCAAACATACATTGCTAAGGCTATTCCTATATCAGCTGGACA
CATATAAAATGCTGCTAATGCTTCATTACAAACTTATATCCTTTAATTCCAGATGGGGGCAAAGTATGTCCAGGGGT
GAGGAACAATTGAAACATTTGGGCTGGAGTAGATTTTGAAAGTCAGCTCTGTGTGTGTGTGTGTGTGTGTGTGTGTC
AGCGTGTGTTTCTTTTAACGTCTTCAGCCTACAACATACAGGGTTCATGGTGGGAAGAAGATAGCAAGATTTAAATT
ATGGCCAGTGACTAGTGCTTGAAGGGGAACAACTACCTGCATTTAATGGGAAGGCAAAATCTCAGGCTTTGAGGGAA
GTTAACATAGGCTTGATTCTGGGTGGAAGCTGGGTGTGTAGTTATCTGGAGGCCAGGCTGGAGCTCTCAGCTCACTA
TGGGTTCATCTTTATTGTCTCCTTTCATCTCAACAGCTCCTGGGAAATGTGCTGGTGACCGTTTTGGCAATCCATTT
CGGCAAAGAATTCACCCCTGAGGTGCAGGCTTCCTGGCAGAAGATGGTGACTGCAGTGGCCAGTGCCCTGTCCTCCA
GATACCACTGAGCCTCTTGCCCATGATTCAGAGCTTTCAAGGATAGGCTTTATTCTGCAAGCAATACAAATAATAAA
TCTATTCTGCTGAGAGATCACACATGATTTTCTTCAGCTCTTTTTTTTACATCTTTTTAAATATATGAGCCACAAAG
GGTTTATATTGAGGGAAGTGTGTATGTGTATTTCTGCATGCCTGTTTGTGTTTGTGGTGTGTGCATGCTCCTCATTT
ATTTTTATATGAGATGTGCATTTTGATGAGCAAATAAAAGCAGTAAAGACACTTGTACACGGGAGTTCTGCAAGTGG
GAGTAAATGGTGTTGGAGAAATCCGGTGGGAAGAAAGACCTCTATAGGACAGGACTTCTCAGAAACAGATGTTTTGG
AAGAGATGGGAAAAGGTTCAGTGAAGACCTGGGGGCTGGATTGATTGCAGCTGAGTAGCAAGGATGGTTCTTAATGA
AGGGAAAGTGTTCCAAGCTTTAGGAATTCAAGGTTTAGTCAGGTGTAGCAATTCTATTTTATTAGGAGGAATACTAT
TTCTAATGGCACTTAGCTTTTCACAGCCCTTGTGGATGCCTAAGAAAGTGAAATTAATCCCATGCCCTCAAGTGTGC
AGATTGGTCACAGCATTTCAAGGGAGAGACCTCATTGTAAGACTCTGGGGGAGGTGGGGACTTAGGTGTAAGAAATG
AATCAGCAGAGGCTCACAAGTCAGCATGAGCATGTTATGTCTGAGAAACAGACCAGCACTGTGAGATCAAAATGTAG
TGGGAAGAATTTGTACAACATTAATTGGAAGGTTTACTTAATGGAATTTTTGTATAGTTGGATGTTAGTGCATCTCT
ATAAGTAAGAGTTTAATATGATGGTGTTACGGACCTGGTGTTTGTGTCTCCTCAAAATTCACATGCTGAATCCCCAA
CTCCCAACTGACCTTATCTGTGGGGGAGGCTTTTGAAAAGTAATTAGGTTTAGCTGAGCTCATAAGAGCAGATCCCC
ATCATAAAATTATTTTCCTTATCAGAAGCAGAGAGACAAGCCATTTCTCTTTCCTCCCGGTGAGGACACAGTGAGAA
GTCCGCCATCTGCAATCCAGGAAGAGAACCCTGACCACGAGTCAGCCTTCAGAAATGTGAGAAAAAACTCTGTTGTT
GAAGCCACCCAGTCTTTTGTATTTTGTTATAGCACCTTACACTGAGTAAGGCAGATGAAGAAGGAGAAAAAAATAAG
CTTGGGTTTTGAGTGAACTACAGACCATGTTATCTCAGGTTTGCAAAGCTCCCCTCGTCCCCTATGTTTCAGCATAA
AATACCTACTCTACTACTCTCATCTATAAGACCCAAATAATAAGCCTGCGCCCTTCTCTCTAACTTTGATTTCTCCT
ATTTTTACTTCAACATGCTTTACTCTAGCCTTGTAATGTCTTTACATACAGTGAAATGTAAAGTTCTTTATTCTTTT
TTTCTTTCTTTCTTTTTTCTCCTCAGCCTCAGAATTTGGCACATGCCCTTCCTTCTTTCAGGAACTTCTCCAACATC
TCTGCCTGGCTCCATCATATCATAAAGGTCCCACTTCAAATGCAGTCACTACCGTTTCAGGATATGCACTTTCTTTC
TTTTTTGTTTTTTGTTTTTTTTAAGTCAAAGCAAATTTCTTGAGAGAGTAAAGAAATAAACGAATGACTACTGCATA
GGCAGAGCAGCCCCGAGGGCCGCTGGTTGTTCCTTTTATGGTTATTTCTTGATGATATGTTAAACAAGTTTTGGATT
ATTTATGCCTTCTCTTTTTAGGCCATATAGGGTAACTTTCTGACATTGCCATGGCATGTTTCTTTTAATTTAATTTA
CTGTTACCTTAAATTCAGGGGTACACGTACAGGATATGCAGGTTTGTTTTATAGGTAAAAGTGTGCCATGGTTTTAA
TGGGTTTTTTTTTTCTTGTAAAGTTGTTTAAGTTTCTTGTTTACTCTGGATATTGGCCTTTGTCAGAAGAATAGATT
GGAAAATCTTTTTCCCATTCTGTAGATTGTCTTTCGCTCTGATGGTAGTTTCTTTTGCTGAGCAGGAGCTCTTTAGT
TTAATTAGATTCCATTGGTCAATTTTTGCTTTTGCTGCAATTGCTTTTCACGCTTTCATCATGAAATCTGTGCCCGT
GTTTATATCATGAATAGTATTGCCTTGATTTTTTTCTAGGCTTTTTATAGTTTGGGGTTTTTCATTTAAGTCTCTAA
TCCATCCGGAGTTAATTTTGGATAAGGTATAAGGAAGGAGTCCAGTTTCATTTTTCAGCATATGGCTAGCCAGTTCT
CCCCCATCATTTATTAAATTGAAAATCCTTTCCCCATTGCTTGCTTTTGTCAGGTTTCTAAAAGACAGATGGTTGTA
GGTACAATATGCAGTTTCTTCAAGTCATATAATACCATCTGAAATCTCTTATTAATTCATTTCTTTTAGTATGTATG
CTGGTCTCCTCTGCTCACTATAGTGAGGGCACCATTAGCCAGAGAATCTGTCTGTCTAGTTCATGTAAGATTCTCAG
AATTAAGAAAAATGGATGGCATATGAATGAAACTTCATGGATGACATATGGAATCTAATGTGTATTTGTTGAATTAA
TGCATAAGATGCAACAAGGGAAAGGTTGACAACTGCAGTGATAACCTGGTATTGATGATATAAGAGTCTATAGATCA
CAGTAGAAGCAATAATCATGGAAAACAATTGGAAATGGGGAACAGCCACAAACAAGAAAGAATCAATACTACCAGGA
AAGTGACTGCAGGTCACTTTTCCTGGAGCGGGTGAGAGAAAAGTGGAAGTTGCAGTAACTGCCGAATTCCTGGTTGG
CTGATGGAAAGATGGGGCAACTGTTCACTGGTACGCAGGGTTTTAGATGTATGTACCTAAGGATATGAGGTATGGCA
ATGAACAGAAATTCTTTTGGGAATGAGTTTTAGGGCCATTAAAGGACATGACCTGAAGTTTCCTCTGAGGCCAGTCC
CCACAACTCAATATAAATGTGTTTCCTGCATATAGTCAAAGTTGCCACTTCTTTTTCTTCATATCATCGATCTCTGC
TCTTAAAGATAATCTTGGTTTTGCCTCAAACTGTTTGTCACTACAAACTTTCCCCATGTTCCTAAGTAAAACAGGTA
ACTGCCTCTCAACTATATCAAGTAGACTAAAATATTGTGTCTCTAATATCAGAAATTCAGCTTTAATATATTGGGTT
TAACTCTTTGAAATTTAGAGTCTCCTTGAAATACACATGGGGGTGATTTCCTAAACTTTATTTCTTGTAAGGATTTA
TCTCAGGGGTAACACACAAACCAGCATCCTGAACCTCTAAGTATGAGGACAGTAAGCCTTAAGAATATAAAATAAAC
TGTTCTTCTCTCTGCCGGTGGAAGTGTGCCCTGTCTATTCCTGAAATTGCTTGTTTGAGACGCATGAGACGTGCAGC
ACATGAGACACGTGCAGCAGCCTGTGGAATATTGTCAGTGAAGAATGTCTTTGCCTGATTAGATATAAAGACAAGTT
AAACACAGCATTAGACTATAGATCAAGCCTGTGCCAGACACAAATGACCTAATGCCCAGCACGGGCCACGGAATCTC
CTATCCTCTTGCTTGAACAGAGCAGCACACTTCTCCCCCAACACTATTAGATGTTCTGGCATAATTTTGTAGATATG
TAGGATTTGACATGGACTATTGTTCAATGATTCAGAGGAAATCTCCTTTGTTCAGATAAGTACACTGACTACTAAAT
GGATTAAAAAACACAGTAATAAAACCCAGTTTTCCCCTTACTTCCCTAGTTTGTTTCTTATTCTGCTTTCTTCCAAG
TTGATGCTGGATAGAGGTGTTTATTTCTATTCTAAAAAGTGATGAAATTGGCCGGGCGCGGTGGCTCACACCTGTAA
TCCCAGCACTTTGGGAGGCTGAGGTGGGCGGATCACGAGGTCAGGAGATCAAGACCATCCTGGCTAACATGGTGAAA
CCCCATCTCTACTAAAAATACAAAAAATTAGCCAGAGACGGTGGCGGGTGCCTGTAGTCCCAGCTACTCGGGAGGCT
GAGGCAGGAGAATGGCGTGAACCTGGGAGGCAGAGCTGCAGTGAGCAGAGATCGCGCCACTGCACACTCCAGCCTGG
GTGACAAAGCGAGACTCCATCTCAAAAAAAAAAAAAAAAAAAAAAAGAAAGAAAGAAAGAAAAAAAAAGTGATGAAA
TTGTGTATTCAATGTAGTCTCAAGAGAATTGAAAACCAAGAAAGGCTGTGGCTTCTTCCACATAAAGCCTGGATGAA
TAACAGGATAACACGTTGTTACATTGTCACAACTCCTGATCCAGGAATTGATGGCTAAGATATTCGTAATTCTTATC
CTTTTCAGTTGTAACTTATTCCTATTTGTCAGCATTCAGGTTATTAGCGGCTGCTGGCGAAGTCCTTGAGAAATAAA
CTGCACACTGGATGGTGGGGGTAGTGTAGGAAAATGGAGGGGAAGGAAGTAAAGTTTCAAATTAAGCCTGAACAGCA
AAGTTCCCCTGAGAAGGCCACCTGGATTCTATCAGAAACTCGAATGTCCATCTTGCAAAACTTCCTTGCCCAAACCC
CACCCCTGGAGTCACAACCCACCCTTGACCAATAGATTCATTTCACTGAGGGAGGCAAAGGGCTGGTCAATAGATTC
ATTTCACTGGGAGAGGCAAAGGGCTGGGGGCCAGAGAGGAGAAGTAAAAAGCCACACATGAAGCAGCAATGCAGGCA
TGCTTCTGGCTCATCTGTGATCACCAGGAAACTCCCAGATCTGACACTGTAGTGCATTTCACTGCTGACAAGAAGGC
TGCTGCCACCAGCCTGTGAAGCAAGGTTAAGGTGAGAAGGCTGGAGGTGAGATTCTGGGCAGGTAGGTACTGGAAGC
CGGGACAAGGTGCAGAAAGGCAGAAAGTGTTTCTGAAAGAGGGATTAGCCCGTTGTCTTACATAGTCTGACTTTGCA
CCTGCTCTGTGATTATGACTATCCCACAGTCTCCTGGTTGTCTACCCATGGACCTAGAGGTACTTTGAAAGTTTTGG
ATATCTGGGCTCTGACTGTGCAATAATGGGCAACCCCAAAGTCAAGGCACATGGCAAGAAGGTGCTGATCTCCTTCG
GAAAAGCTGTTATGCTCACGGATGACCTCAAAGGCACCTTTGCTACACTGAGTGACCTGCACTGTAACAAGCTGCAC
GTGGACCCTGAGAACTTCCTGGTGAGTAGTAAGTACACTCACGCTTTCTTCTTTACCCTTAGATATTTGCACTATGG
GTACTTTTGAAAGCAGAGGTGGCTTTCTCTTGTGTTATGAGTCAGCTATGGGATATGATATTTCAGCAGTGGGATTT
TGAGAGTTATGTTGCTGTAAATAACATAACTAAAATTTGGTAGAGCAAGGACTATGAATAATGGAAGGCCACTTACC
ATTTGATAGCTCTGAAAAACACATCTTATAAAAAATTCTGGCCAAAATCAAACTGAGTGTTTTGGATGAGGGAACAG
AAGTTGAGATAGAGAAAATAACATCTTTCCTTTGGTCAGCGAAATTTTCTATAAAAATTAATAGTCACTTTTCTGCA
TAGTCCTGGAGGTTAGAAAAAGATCAACTGAACAAAGTAGTGGGAAGCTGTTAAAAGAGGATTGTTTCCCTCCGAAT
GATGATGGTATACTTTTGTACGCATGGTACAGGATTCTTTGTTATGAGTGTTTGGGAAAATTGTATGTATGTATGTA
TGTATGTGATGACTGGGGACTTATCCTATCCATTACTGTTCCTTGAAGTACTATTATCCTACTTTTTAAAAGGACGA
AGTCTCTAAAAAAAAAATGAAACAATCACAATATGTTGGGGTAGTGAGTTGGCATAGCAAGTAAGAGAAGGATAGGA
CACAATGGGAGGTGCAGGGCTGCCAGTCATATTGAAGCTGATATCTAGCCCATAATGGTGAGAGTTGCTCAAACTCT
GGTCAAAAAGGATGTAAGTGTTATATCTATTTACTGCAAGTCCAGCTTGAGGCCTTCTATTCACTATGTACCATTTT
CTTTTTTATCTTCACTCCCTCCCCAGCTCTTAGGCAACGTGATATTGATTGTTTTGGCAACCCACTTCAGCGAGGAT
TTTACCCTACAGATACAGGCTTCTTGGCAGTAACTAACAAATGCTGTGGTTAATGCTGTAGCCCACAAGACCACTGA
GTTCCCTGTCCACTATGTTTGTACCTATGTCCCAAAATCTCATCTCCTTTAGATGGGGGAGGTTGGGGAGAAGAGCA
GTATCCTGCCTGCTGATTCAGTTCCTGCATGATAAAAATAGAATAAAGAAATATGCTCTCTAAGAAATATCATTGTA
CTCTTTTTCTGTCTTTATATTTTACCCTGATTCAGCCAAAAGGACGCACTATTTCTGATGGAAATGAGAATGTTGGA
GAATGGGAGTTTAAGGACAGAGAAGATACTTTCTTGCAATCCTGCAAGAAAAGAGAGAACTCGTGGGTGGATTTAGT
GGGGTAGTTACTCCTAGGAAGGGGAAATCGTCTCTAGAATAAGACAATGTTTTTACAGAAAGGGAGGTCAATGGAGG
TACTCTTTGGAGGTGTAAGAGGATTGTTGGTAGTGTGTAGAGGTATGTTAGGACTCAAATTAGAAGTTCTGTATAGG
CTATTATTTGTATGAAACTCAGGATATAGCTCATTTGGTGACTGCAGTTCACTTCTACTTATTTTAAACAACATATT
TTTTATGATTTATAATGAAGTGGGGATGGGGCTTCCTAGAGACCAATCAAGGGCCAAACCTTGAACTTTCTCTTAAC
GTCTTCAATGGTATTAATAGAGAATTATCTCTAAGGCATGTGAACTGGCTGTCTTGGTTTTCATCTGTACTTCATCT
GCTACCTCTGTGACCTGAAACATATTTATAATTCCATTAAGCTGTGCATATGATAGATTTATCATATGTATTTTCCT
TAAAGGATTTTTGTAAGAACTAATTGAATTGATACCTGTAAAGTCTTTATCACACTACCCAATAAATAATAAATCTC
TTTGTTCAGCTCTCTGTTTCTATAAATATGTACAAGTTTTATTGTTTTTAGTGGTAGTGATTTTATTCTCTTTCTAT
ATATATACACACACATGTGTGCATTCATAAATATATACAATTTTTATGAATAAAAAATTATTAGCAATCAATATTGA
AAACCACTGATTTTTGTTTATGTGAGCAAACAGCAGATTAAAAGGCTGAGATTTAGGAAACAGCACGTTAAGTCAAG
TTGATAGAGGAGAATATGGACATTTAAAAGAGGCAGGATGATATAAAATTAGGGAAACTGGATGCAGAGACCAGATG
AAGTAAGAAAAATAGCTATCGTTTTGAGCAAAAATCACTGAAGTTTCTTGCATATGAGAGTGACATAATAAATAGGG
AAACGTAGAAAATTGATTCACATGTATATATATATATAGAACTGATTAGACAAAGTCTAACTTGGGTATAGTCAGAG
GAGCTTGCTGTAATTATATTGAGGTGATGGATAAAGAACTGAAGTTGATGGAAACAATGAAGTTAAGAAAAAAAATC
GAGTAAGAGACCATTGTGGCAGTGATTGCACAGAACTGGAAAACATTGTGAAACAGAGAGTCAGAGATGACAGCTAA
AATCCCTGTCTGTGAATGAAAAGAAGGAAATTTATTGACAGAACAGCAAATGCCTACAAGCCCCCTGTTTGGATCTG
GCAATGAACGTAGCCATTCTGTGGCAATCACTTCAAACTCCTGTACCCAAGACCCTTAGGAAGTATGTAGCACCCTC
AAACCTAAAACCTCAAAGAAAGAGGTTTTAGAAGATATAATACCCTTTCTTCTCCAGTTTCATTAATCCCAAAACCT
CTTTCTCAAAGTATTTCCTCTATGTGTCCACCCCAAAGAGCTCACCTCACCATATCTCTTGAGTGGGAGCACATAGA
TAGGCGGTGCTACCATCTAACAGCTTCTGAAATTCCTTTGTCATATTTTTGAGTCCCCACTAATAACCCACAAAGCA
GAATAAATACCAGTTGCTCATGTACAATAATCACTCAACTGCTGTCTTGTAGCATACATTAATTAAGCACATTCTTT
GAATAATTACTGTGTCCAAACAATCACACTTTAAAATCTCACACTTGTGCTATCCCTTGCCCTTCTGAATGTCACTC
TGTATTTTAAATGAAGAGATGAGGGTTGAATTTCCTGTGTTACTTATTGTTCATTTCTCGATGAGGAGTTTTCACAT
TCACCTTTACTGGAAAACACATAAGTACACATCTTACAGGAAAAATATACCAAACTGACATGTAGCATGAATGCTTG
TGCATGTAGTCATATAAAATCTTGTAGCAATGTAAACATTCTCTGATATACACATACAGATGTGTCTATATGTCTAC
ACAATTTCTTATGCTCCATGAACAAACATTCCATGCACACATAAGAACACACACTGTTACAGATGCATACTTGAGTG
CATTGACAAAATTACCCCAGTCAATCTAGAGAATTTGGATTTCTGCATTTGACTCTGTTAGCTTTGTACATGCTGTT
CATTTACTCTGGGTGATGTCTTTCCCTCATTTTGCCTTGTCTATCTTGTACTCATACTTTAAGTCCTAACTTATATG
TTATCTCAACTAAGAAGCTATTTTTTTTTAATTTTAACTGGGCTTAAAGCCCTGTCTATAAACTCTGCTACAATTAT
GGGCTCTTTCTTATAATATTTAGTGTTTTTCCTACTAATGTACTTAATCTGCTCATTGTATATTCCTACCACTAAAT
TTTAACCTCTTTTATGGTAGAGACATTGTCTTGTAAACTCTTATTTCCCTAGTATTTGGAGATGAAAAAAAAGATTA
AATTATCCAAAATTAGATCTCTCTTTTCTACATTATGAGTATTACACTATCCATAGGGAAGTTTGTTTGAGACCTAA
ACTGAGGAACCTTTGGTTCTAAAATGACTATGTGATATCTTAGTATTTATAGGTCATGAGGTTCCTTCCTCTGCCTC
TGCTATAGTTTGATTAGTCAGCAAGCATGTGTCATGCATTTATTCACATCAGAATTTCATACACTAATAAGACATAG
TATCAGAAGTCAGTTTATTAGTTATATCAGTTAGGGTCCATCAAGGAAAGGACAAACCATTATCAGTTACTCAACCT
AGAATTAAATACAGCTCTTAATAGTTAATTATCCTTGTATTGGAAGAGCTAAAATATCAAATAAAGGACAGTGCAGA
AATCTAGATGTTAGTAACATCAGAAAACCTCTTCCGCCATTAGGCCTAGAAGGGCAGAAGGAGAAAATGTTTATACC
ACCAGAGTCCAGAACCAGAGCCCATAACCAGAGGTCCACTGGATTCAGTGAGCTAGTGGGTGCTCCTTGGAGAGAGC
CAGAACTGTCTAATGGGGGCATCAAAGTATCAGCCATAAAAAACCATAAAAAAGACTGTCTGCTGTAGGAGATCCGT
TCAGAGAGAGAGAGAGACCAGAAATAATCTTGCTTATGCTTTCCCTCAGCCAGTGTTTACCATTGCAGAATGTACAT
GCGACTGAAAGGGTGAGGAAACCTGGGAAATGTCAGTTCCTCAAATACAGAGAACACTGAGGGAAGGATGAGAAATA
AATGTGAAAGCAGACATGAATGGTAATTGACAGAAGGAAACTAGGATGTGTCCAGTAAATGAATAATTACAGTGTGC
AGTGATTATTGCAATGATTAATGTATTGATAAGATAATATGAAAACACAGAATTCAAACAGCAGTGAACTGAGATTA
GAATTGTGGAGAGCACTGGCATTTAAGAATGTCACACTTAGAATGTGTCTCTAGGCATTGTTCTGTGCATATATCAT
CTCAATATTCATTATCTGAAAATTATGAATTAGGTACAAAGCTCAAATAATTTATTTTTTCAGGTTAGCAAGAACTT
TTTTTTTTTTTTTTTCTGAGATGGAGCATTGCTATGGTTGCCCAGGCTGGAGTGCAATGGCATGATCCAGGCTCACT
GCAACATCTGCCTCCCAGGTTCAAGCGATTCTCCTGCCTCAGCCTCCCAAGTAGCTGGCATTACAGGCATGTGCCAC
CACCATGCCTGGCTAATTTTCTATTTTTAGTAGATAGGGGGTTTCACCATGTTGGTCAGGCTGATCTCGAACTCCTA
ACATCAGGTGATCCACCCTCCTCGGCCTCTGAATGTACTGGGATCACAGGCGTGAGCCACCACACCCAGCCAAGAAT
GTGAATTTTGTAGAAGGATATAACCCATATTTCTCTGACCCTAGAGTCCTTAGTATACCTCCCATACCATGTGGCTC
ATCCTCCTTACATACATTTCCCATCTTTCACCCTACCTTTTCCTTTTTGTTTCAGCTTTTCACTGTGTGTCAAAATC
TAGAACCTTATCTCCTACCTGCTCTGAAACCAACAGCAAGTTGACTTCCATTCTAACCCACATTGGCATTACACTAA
TTAAAATCGATACTGAGTTCTAAAATCATCTGGGATTTTGGGGACTATGTCTTACTTCATACTTCCTTGAGATTTCA
CATTAAATGTTGGTGTTCATTAAAGGTCCTTCATTTAACTTTGTATTCATCACACTCTTGGATTCACAGTTATATCT
AAACTCTTATATATAGCCTGTATAATCCCAATTCCCAAGTCTGATTTCTAACCTCTGACCTCCAACCTCAGTGCCAA
ACCCATATATCAAACAATGTACTGGGCTTATTTATATAGATGTCCTATAGGCACCTCAGACTCAGCATGGGTATTTC
ACTTGTTATACTAAAACTGTTTCTCTTCCAGTGTTTTCCATTTTAGTCATTAGATAGCTACTTGCCCATTCACCAAG
GTCACAGATTAAAATCATTTCCCTACCTCTAATCAACAGTTCAATTCTGCTTCAATTTGTCCCTATCTATTAATCAC
CACTCTTACTGCCCAGTCAGGTCCTCATTGTTTCCTGAACAAGAGTAGATGCTATTCTTTCCACTTTAAGACCTTAT
CCTGGCTGGATGCGGTGGCTCAGGCTTGTAAACCCAGCACTTTGGGAGGCCGAGGCAGGCAGATCACTTGAGGTCAG
GAGTTCAAGACCAGCCTGACCAACATGGTGAAACCCCATCTCTACTAAAAATACAAAATCAGCCGGGCGTGTGGTGC
ATGCCTGCAGTCCCAGCTATTCAGGTGGCTGAGGCAGGAGAATTGCTTGAACCCAGGAGGCGGAGGTTGCGGTGAGC
CTAGATTGCACCATTGCACTCTAGCTTGGGCAATAGGGATGAAACTCCATCTCAGAAGAGAAAAGAAAAAAAGACCT
TATTCTGTTACACAAATCCTCTCAATGCAATCCATATAGAATAAACATGTAACCAGATCTCCCAATGTGTAAAATCA
TTTCAGGTAGAACAGAATTAAAGTGAAAAGCCAAGTCTTTGGAATTAACAGACAAAGTTCAAATAACAGTCCTCATG
GCCTTAAGAATTTACCTAACATTTTTTTTAGAATCAATTTTCTTATATATGAATTGGAAACATAATTCCTCCCTCAC
AAACACATTCTAAGATTTTAAGGAGATATTGATGAAGTACATCATCTGTCATTTTTAACAGTTAGTGGTAGTGATTC
ACACAGCACATTATGATCTGTTCTTGTATGTTCTGTTCCATTCTGTATTCTTGACCTGGTTGTATTCTTTCTGAGCT
CCAGATCCACATATCTAAGTACATCTTTTTGCATTTTACAAGAGTGCATACAATACAATGTATCCAAGACTGTATTT
CTGATTTTATCGTACCACTAAACTCACAAATGTGGCCCTATTCTTGTGTTCACGACTGACATCACCGTCATGGTCCA
AGTCTGATAATAGAAATGGCATTGTCACTTTCTTCCCTACTGCAACAGAAGCCCAGCTATTTGTCTCCCATTTTCTC
TACTTCTAAAATACATTTCTTCACTAAGTGAGAATAATCTTTTAAAGACACAAATCAAACCATGCCACCACCTTTCT
TGAATTATTCAATATCTTTCGTTGGCTTCCAGGTTACAGAAAAATAACTTGTAACAAAGTTTAAAGGTCATTCATGG
CTCCTCTCTACCCTATTTTATAACATTTCCCCTTGTGATCAGAATCTCAGGCACATCATCCATCTTTCTATATACAA
ATAAAGTCATATAGTTTGAACTCACCTCTGGTTACTTTTAATCAACCAAATGCTGTAAAATGCATTTGTATCGCTAC
GTGTTAAGCAGTAGTTGATTCTTTTCATTTCTTGTTAATATTCTATTCTTTGACTATACCGTAATTTATCAATTCTA
CTGTTGGTAAGCATTTAAGTGGCTACCGGTTTGAGGTTTTTATGATTATTGCTGTCATAAGCATTTCTATACATGTC
TTTGGATACACACATGCATGTGTTTCTGAATATCTAAAAATGTAATTGCTAGGTAATAGACTTATCAAGCATCCAGC
ATTTGTGGATACTATTAAAGGTTTTCCAAAGGGGTTATACTATTGTACAGTGTCACCAACAGAGTTTGAGTTTCTAT
TGATCCATATCACCACCAAAATTTGAACTGTCAGTCTTATCTCTTCTCTTGTCTCTTTTTTCCTCTTTTTTTTCCTT
CCCTTCCCCTCTCTTCGTTTCTTTTCTCTCCTCTTCTCTTCTTTCCTCTCTTCCCTTCCCTTTCTCTTTCTCTTCCC
TATCCCTTCTCCTCTCCTCTCCCCTCCTTTTTTCTCCTCTCCTCTCCATTATTTATTTTTCCTTCTTCTCCTCCATC
CCTTCCATCCTCTCTCTTCCCCTCTTCCTTCCTTCCTTTCTCCATTTCTTCCTCCTCTTTCCCTCAATCCTTCCTTT
TGGATATGCTCATGGGTGTGTATTTGTCTGCCATTGTGGCATTATTTGAATTCAGAAAAGAGTGAAAAACTACTGGG
ATCTTCATTCTGGGTCTAATTCCACATTTTTTTTTAAGAACACACTCTGTAAAAATGTTCTGTACTAGCATATTCCC
AGGAACTTCGTTAAATTTAATCTGGCTGAATATGGTAAATCTACTTTGCACTTTGCATTCTTTCTTTAGTCATACCA
TAATTTTAAACATTCAAAATATTTGTATATAATATTTGATTTTATCTGTCATTAAAATGTTAACCTTAAAATTCATG
TTTCCAGAACCTATTTCAATAACTGGTAAATAAACACTATTCATTTTTTAAATATTCTTTTAATGGATATTTATTTC
AATATAATAAAAAATTAGAGTTTTATTATAGGAAGAATTTACCAAAAGAAGGAGGAAGCAAGCAAGTTTAAACTGCA
GCAATAGTTGTCCATTCCAACCTCTCAAAATTCCCTTGGAGACAAAATCTCTAGAGGCAAAGAAGAACTTTATATTG
AGTCAACTTGTTAAAACATCTGCTTTTAGATAAGTTTTCTTAGTATAAAGTGACAGAAACAAATAAGTTAAACTCTA
AGATACATTCCACTATATTAGCCTAAAACACTTCTGCAAAAATGAAACTAGGAGGATATTTTTAGAAACAACTGCTG
AAAGAGATGCGGTGGGGAGATATGCAGAGGAGAACAGGGTTTCTGAGTCAAGACACACATGACAGAACAGCCAATCT
CAGGGCAAGTTAAGGGAATAGTGGAATGAAGGTTCATTTTTCATTCTCACAAACTAATGAAACCCTGCTTATCTTAA
ACCAACCTGCTCACTGGAGCAGGGAGGACAGGACCAGCATAAAAGGCAGGGCAGAGTCGACTGTTGCTTACACTTTC
TTCTGACATAACAGTGTTCACTAGCAACCTCAAACAGACACCATGGTGCATCTGACTCCTGAGGAGAAGACTGCTGT
CAATGCCCTGTGGGGCAAAGTGAACGTGGATGCAGTTGGTGGTGAGGCCCTGGGCAGGTTGGTATCAAGGTTATAAG
AGAGGCTCAAGGAGGCAAATGGAAACTGGGCATGTGTAGACAGAGAAGACTCTTGGGTTTCTGATAGGCACTGACTC
TCTGTCCCTTGGGCTGTTTTCCTACCCTCAGATTACTGGTGGTCTACCCTTGGACCCAGAGGTTCTTTGAGTCCTTT
GGGGATCTGTCCTCTCCTGATGCTGTTATGGGCAACCCTAAGGTGAAGGCTCATGGCAAGAAGGTGCTAGGTGCCTT
TAGTGATGGCCTGGCTCACCTGGACAACCTCAAGGGCACTTTTTCTCAGCTGAGTGAGCTGCACTGTGACAAGCTGC
ACGTGGATCCTGAGAACTTCAGGGTGAGTCCAGGAGATGCTTCACTTTTCTCTTTTTACTTTCTAATCTTACATTTT
GGTTCTTTTACCTACCTGCTCTTCTCCCACATTTTTGTCATTTTACTATATTTTATCATTTAATGCTTCTAAAATTT
TGTTAATTTTTTATTTAAATATTCTGCATTTTTTCCTTCCTCACAATCTTGCTATTTTAAATTATTTAATATCCTGT
CTTTCTCTCCCAACCCCCTCCCTTCATTTTTCCTTCTCTAACAACAACTCAAATTATGCATACCAGCTCTCACCTGC
TAATTCTGCACTTAGAATAATCCTTTTGTCTCTCCACATGGGTATGGGAGAGGCTCCAACTCAAAGATGAGAGGCAT
AGAATACTGTTTTAGAGGCTATAAATCATTTTACAATAAGGAATAATTGGAATTTTATAAATTCTGTAGTAAATGGA
ATGGAAAGGAAAGTGAATATTTGATTATGAAAGACTAGGCAGTTACACTGGAGGTGGGGCAGAAGTCGTTGCTAGGA
GACAGCCCATCATCACACTGATTAATCAATTAATTTGTATCTATTAATCTGTTTATAGTAATTAATTTGTATATGCT
ATATACACATACAAAATTAAAACTAATTTGGAATTAATTTGTATATAGTATTATACAGCATATATAGCATATATGTA
CATATATAGACTACATGCTAGTTAAGTACATAGAGGATGTGTGTGTATAGATATATGTTATATGTATGCATTCATAT
ATGTACTTATTTATGCTGATGGGAATAACCTGGGGATCAGTTTTGTCTAAGATTTGGGCAGAAAAAAATGGGTGTTG
GCTCAGTTTCTCAGAAGCCAGTCTTTATTTCTCTGTTAACCATATGCATGTATCTGCCTACCTCTTCTCCGCAGCTC
TTGGGCAATGTGCTGGTGTGTGTGCTGGCCCGCAACTTTGGCAAGGAATTCACCCCACAAATGCAGGCTGCCTATCA
GAAGGTGGTGGCTGGTGTGGCTAATGCCCTGGCTCACAAGTACCATTGAGATCCTGGACTGTTTCCTGATAACCATA
AGAAGACCCTATTTCCCTAGATTCTATTTTCTGAACTTGGGAACACAATGCCTACTTCAAGGGTATGGCTTCTGCCT
AATAAAGAATGTTCAGCTCAACTTCCTGATTAATTTCACTTATTTCATTTTTTTGTCCAGGTGTGTAAGAAGGTTCC
TGAGGCTCTACAGATAGGGAGCACTTGTTTATTTTACAAAGAGTACATGGGAAAAGAGAAAAGCAAGGGAACCGTAC
AAGGCATTAATGGGTGACACTTCTACCTCCAAAGAGCAGAAATTATCAAGAACTCTTGATACAAAGATAATACTGGC
ACTGCAGAGGTTCTAGGGAAGACCTCAACCCTAAGACATAGCCTCAAGGGTAATAGCTACGATTAAACTCCAACAAT
TACTGAGAAAATAATGTGCTCAATTAAAGGCATAATGATTACTCAAGACAATGTTATGTTGTCTTTCTTCCTCCTTC
CTTTGCCTGCACATTGTAGCCCATAATACTATACCCCATCAAGTGTTCCTGCTCCAAGAAATAGCTTCCTCCTCTTA
CTTGCCCCAGAACATCTCTGTAAAGAATTTCCTCTTATCTTCCCATATTTCAGTCAAGATTCATTGCTCACGTATTA
CTTGTGACCTCTCTTGACCCCAGCCACAATAAACTTCTCTATACTACCCAAAAAATCTTTCCAAACCCTCCCCGACA
CCATATTTTTATATTTTTCTTATTTATTTCATGCACACACACACACTCCGTGCTTTATAAGCAATTCTGCCTATTCT
CTACCTTCTTACAATGCCTACTGTGCCTCATATTAAATTCATCAATGGGCAGAAAGAAAATATTTATTCAAGAAAAC
AGTGAATGAATGAACGAATGAGTAAATGAGTAAATGAAGGAATGATTATTCCTTGCTTTAGAACTTCTGGAATTAGA
GGACAATATTAATAATACCATCGCACAGTGTTTCTTTGTTGTTAATGCTACAACATACAAAGAGGAAGCATGCAGTA
AACAACCGAACAGTTATTTCCTTTCTGATCATAGGAGTAATATTTTTTTCCTTGAGCACATTTTTGCCATAGGTAAA
ATTAGAAGGATTTTTAGAACTTTCTCAGTTGTATACATTTTTAAAAATCTGTATTATATGCATGTTGATTAATTTTA
AACTTACTTGAATACCTAAACAGAATCTGTTGTTTCCTTGTGTTTGAAAGTGCTTTCACAGTAACTCTGTCTGTACT
GCCAGAATATACTGACAATGTGTTATAGTTAACTGTTTTGATCACAACATTTTGAATTGACTGGCAGCAGAAGCTCT
TTTTATATCCATGTGTTTTCCTTAAGTCATTATACATAGTAGGCATGAGACTCTTTATACTGAATAAGATATTTAGG
AACCACTGGTTTACATATCAGAAGCAGAGCTACTCAGGGCATTTTGGGGAAGATCACTTTCACATTCCTGAGCATAG
GGAAGTTCTCATAAGAGTAAGATATTAAAAGGAGATACTTGTGTGGTATTCGAAAGACAGTAAGAGAGATTGTAGAC
CTTATGATCTTGATAGGGAAAACAAACTACATTCCTTTCTCCAAAAGTCAAAAAAAAAGAGCAAATATAGCTTACTA
TACCTTCTATTCCTACACCATTAGAAGTAGTCAGTGAGTCTAGGCAAGATGTTGGCCCTAAAAATCCAAATACCAGA
GAATTCATGAGAACATCACCTGGATGGGACATGTGCCGAGCAACACAATTACTATATGCTAGGCATTGCTATCTTCA
TATTGAAGATGAGGAGGTCAAGAGATGAAAAAAGACTTGGCACCTTGTTGTTATATTAAAATTATTTGTTAGAGTAG
AGCTTTTGTAAGAGTCTAGGAGTGTGGGAGCTAAATGATGATACACATGGACACAAAGAATAGATCAACAGACACCC
AGGCCTACTTGAGGGTTGAGGGTGGGAAGAGGGAGACGATGAAAAAGAACCTATTGGGTATTAAGTTCATCACTGAG
TGATGAAATAATCTGTACATCAAGACCCAGTGATATGCAATTTACCTATATAACTTGTACATGTACCCCCAAATTTA
AAATAAAGTTAAAACAAAGTATAGGAATGGAATTAATTCCTCAAGATTTGGCTTTAATTTTATTTGATAATTTATCA
AATGGTTGTTTTTCTTTTCTCACTATGGCGTTGCTTTATAAACTATGTTCAGTATGTCTGAATGAAAGGGTGTGTGT
GTGTGTGAAAGAGAGGGAGAGAGGAAGGGAAGAGAGGACGTAATAATGTGAATTTGAGTTCATGAAAATTTTTCAAT
AAAATAATTTAATGTCAGGAGAATTAAGCCTAATAGTCTCCTAAATCATCCATCTCTTGAGCTTCAGAGCAGTCCTC
TGAATTAATGCCTACATGTTTGTAAAGGGTGTTCAGACTGAAGCCAAGATTCTACCTCTAAAGAGATGCAATCTCAA
ATTTATCTGAAGACTGTACCTCTGCTCTCCATAAATTGACACCATGGCCCACTTAATGAGGTTAAAAAAAAGCTAAT
TCTGAATGAAAATCTGAGCCCAGTGGAGGAAATATTAATGAACAAGGTGCAGACTGAAATATAAATTTTCTGTAATA
ATTATGCATATACTTTAGCAAAGTTCTGTCTATGTTGACTTTATTGCTTTTGGTAAGAAATACAACTTTTTAAAGTG
AACTAAACTATCCTATTTCCAAACTATTTTGTGTGTGTGCGGTTTGTTTCTATGGGTTCTGGTTTTCTTGGAGCATT
TTTATTTCATTTTAATTAATTAATTCTGAGAGCTGCTGAGTTGTGTTTACTGAGAGATTGTGTATCTGCGAGAGAAG
TCTGTAGCAAGTAGCTAGACTGTGCTTGACCTAGGAACATATACAGTAGATTGCTAAAATGTCTCACTTGGGGAATT
TTAGACTAAACAGTAGAGCATGTATAAAAATACTCTAGTCAAGTGCTGCTTTTGAAACAAATGATAAAACCACACTC
CCATAGATGAGTGTCATGATTTTCATGGAGGAAGTTAATATTCATCCTCTAAGTATACCCAGACTAGGGCCATTCTG
ATATAAAACATTAGGACTTAAGAAAGATTAATAGACTGGAGTAAAGGAAATGGACCTCTGTCTCTCTCGCTGTCTCT
TTTTTGAGGACTTGTGTGTGTGTGTGTGTGTGTGTGTGTGTGTGTTGTGGTCAGTGGGGCTGGAATAAAAGTAGAAT
AGACCTGCACCTGCTGTGGCATCCATTCACAGAGTAGAAGCAAGCTCACAATAGTGAAGATGTCAGTAAGCTTGAAT
AGTTTTTCAGGAACTTTGAATGCTGATTTAGATTTGAAACTGAGGCTCTGACCATAACCAAATTTGCACTATTTATT
GCTTCTTGAAACTTATTTGCCTGGTATGCCTGGGCTTTTGATGGTCTTAGTATAGCTTGCAGCCTTGTCCCTGCAGG
GTATTATGGGTAATAGAAAGAAAAGTCTGCGTTACACTCTAGTCACACTAAGTAACTACCATTGGAAAAGCAACCCC
TGCCTTGAAGCCAGGATGATGGTATCTGCAGCAGTTGCCAACACAAGAGAAGGATCCATAGTTCATCATTTAAAAAA
GAAAACAAAATAGAAAAAGGAAAACTATTTCTGAGCATAAGAAGTTGTAGGGTAAGTCTTTAAGAAGGTGACAATTT
CTGCCAATCAGGATTTCAAAGCTCTTGCTTTGACAATTTTGGTCTTTCAGAATACTATAAATATAACCTATATTATA
ATTTCATAAAGTCTGTGCATTTTCTTTGACCCAGGATATTTGCAAAAGACATATTCAAACTTCCGCAGAACACTTTA
TTTCACATATACATGCCTCTTATATCAGGGATGTGAAACAGGGTCTTGAAAACTGTCTAAATCTAAAACAATGCTAA
TGCAGGTTTAAATTTAATAAAATAAAATCCAAAATCTAACAGCCAAGTCAAATCTGTATGTTTTAACATTTAAAATA
TTTTAAAGACGTCTTTTCCCAGGATTCAACATGTGAAATCTTTTCTCAGGGATACACGTGTGCCTAGATCCTCATTG
CTTTAGTTTTTTACAGAGGAATGAATATAAAAAGAAAATACTTAAATTTTATCCCTCTTACCTCTATAATCATACAT
AGGCATAATTTTTTAACCTAGGCTCCAGATAGCCATAGAAGAACCAAACACTTTCTGCGTGTGTGAGAATAATCAGA
GTGAGATTTTTTCACAAGTACCTGATGAGGGTTGAGACAGGTAGAAAAAGTGAGAGATCTCTATTTATTTAGCAATA
ATAGAGAAAGCATTTAAGAGAATAAAGCAATGGAAATAAGAAATTTGTAAATTTCCTTCTGATAACTAGAAATAGAG
GATCCAGTTTCTTTTGGTTAACCTAAATTTTATTTCATTTTATTGTTTTATTTTATTTTATTTTATTTTATTTTGTG
TAATCGTAGTTTCAGAGTGTTAGAGCTGAAAGGAAGAAGTAGGAGAAACATGCAAAGTAAAAGTATAACACTTTCCT
TACTAAACCGACTGGGTTTCCAGGTAGGGGCAGGATTCAGGATGACTGACAGGGCCCTTAGGGAACACTGAGACCCT
ACGCTGACCTCATAAATGCTTGCTACCTTTGCTGTTTTAATTACATCTTTTAATAGCAGGAAGCAGAACTCTGCACT
TCAAAAGTTTTTCCTCACCTGAGGAGTTAATTTAGTACAAGGGGAAAAAGTACAGGGGGATGGGAGAAAGGCGATCA
CGTTGGGAAGCTATAGAGAAAGAAGAGTAAATTTTAGTAAAGGAGGTTTAAACAAACAAAATATAAAGAGAAATAGG
AACTTGAATCAAGGAAATGATTTTAAAACGCAGTATTCTTAGTGGACTAGAGGAAAAAAATAATCTGAGCCAAGTAG
AAGACCTTTTCCCCTCCTACCCCTACTTTCTAAGTCACAGAGGCTTTTTGTTCCCCCAGACACTCTTGCAGATTAGT
CCAGGCAGAAACAGTTAGATGTCCCCAGTTAACCTCCTATTTGACACCACTGATTACCCCATTGATAGTCACACTTT
GGGTTGTAAGTGACTTTTTATTTATTTGTATTTTTGACTGCATTAAGAGGTCTCTAGTTTTTTATCTCTTGTTTCCC
AAAACCTAATAAGTAACTAATGCACAGAGCACATTGATTTGTATTTATTCTATTTTTAGACATAATTTATTAGCATG
CATGAGCAAATTAAGAAAAACAACAACAAATGAATGCATATATATGTATATGTATGTGTGTATATATACACATATAT
ATATATATTTTTTTTCTTTTCTTACCAGAAGGTTTTAATCCAAATAAGGAGAAGATATGCTTAGAACTGAGGTAGAG
TTTTCATCCATTCTGTCCTGTAAGTATTTTGCATATTCTGGAGACGCAGGAAGAGATCCATCTACATATCCCAAAGC
TGAATTATGGTAGACAAAGCTCTTCCACTTTTAGTGCATCAATTTCTTATTTGTGTAATAAGAAAATTGGGAAAACG
ATCTTCAATATGCTTACCAAGCTGTGATTCCAAATATTACGTAAATACACTTGCAAAGGAGGATGTTTTTAGTAGCA
ATTTGTACTGATGGTATGGGGCCAAGAGATATATCTTAGAGGGAGGGCTGAGGGTTTGAAGTCCAACTCCTAAGCCA
GTGCCAGAAGAGCCAAGGACAGGTACGGCTGTCATCACTTAGACCTCACCCTGTGGAGCCACACCCTAGGGTTGGCC
AATCTACTCCCAGGAGCAGGGAGGGCAGGAGCCAGGGCTGGGCATAAAAGTCAGGGCAGAGCCATCTATTGCTTACA
TTTGCTTCTGACACAACTGTGTTCACTAGCAACCTCAAACAGACACCATGGTGCACCTGACTCCTGAGGAGAAGTCT
GCCGTTACTGCCCTGTGGGGCAAGGTGAACGTGGATGAAGTTGGTGGTGAGGCCCTGGGCAGGTTGGTATCAAGGTT
ACAAGACAGGTTTAAGGAGACCAATAGAAACTGGGCATGTGGAGACAGAGAAGACTCTTGGGTTTCTGATAGGCACT
GACTCTCTCTGCCTATTGGTCTATTTTCCCACCCTTAGGCTGCTGGTGGTCTACCCTTGGACCCAGAGGTTCTTTGA
GTCCTTTGGGGATCTGTCCACTCCTGATGCTGTTATGGGCAACCCTAAGGTGAAGGCTCATGGCAAGAAAGTGCTCG
GTGCCTTTAGTGATGGCCTGGCTCACCTGGACAACCTCAAGGGCACCTTTGCCACACTGAGTGAGCTGCACTGTGAC
AAGCTGCACGTGGATCCTGAGAACTTCAGGGTGAGTCTATGGGACCCTTGATGTTTTCTTTCCCCTTCTTTTCTATG
GTTAAGTTCATGTCATAGGAAGGGGAGAAGTAACAGGGTACAGTTTAGAATGGGAAACAGACGAATGATTGCATCAG
TGTGGAAGTCTCAGGATCGTTTTAGTTTCTTTTATTTGCTGTTCATAACAATTGTTTTCTTTTGTTTAATTCTTGCT
TTCTTTTTTTTTCTTCTCCGCAATTTTTACTATTATACTTAATGCCTTAACATTGTGTATAACAAAAGGAAATATCT
CTGAGATACATTAAGTAACTTAAAAAAAAACTTTACACAGTCTGCCTAGTACATTACTATTTGGAATATATGTGTGC
TTATTTGCATATTCATAATCTCCCTACTTTATTTTCTTTTATTTTTAATTGATACATAATCATTATACATATTTATG
GGTTAAAGTGTAATGTTTTAATATGTGTACACATATTGACCAAATCAGGGTAATTTTGCATTTGTAATTTTAAAAAA
TGCTTTCTTCTTTTAATATACTTTTTTGTTTATCTTATTTCTAATACTTTCCCTAATCTCTTTCTTTCAGGGCAATA
ATGATACAATGTATCATGCCTCTTTGCACCATTCTAAAGAATAACAGTGATAATTTCTGGGTTAAGGCAATAGCAAT
ATTTCTGCATATAAATATTTCTGCATATAAATTGTAACTGATGTAAGAGGTTTCATATTGCTAATAGCAGCTACAAT
CCAGCTACCATTCTGCTTTTATTTTATGGTTGGGATAAGGCTGGATTATTCTGAGTCCAAGCTAGGCCCTTTTGCTA
ATCATGTTCATACCTCTTATCTTCCTCCCACAGCTCCTGGGCAACGTGCTGGTCTGTGTGCTGGCCCATCACTTTGG
CAAAGAATTCACCCCACCAGTGCAGGCTGCCTATCAGAAAGTGGTGGCTGGTGTGGCTAATGCCCTGGCCCACAAGT
ATCACTAAGCTCGCTTTCTTGCTGTCCAATTTCTATTAAAGGTTCCTTTGTTCCCTAAGTCCAACTACTAAACTGGG
GGATATTATGAAGGGCCTTGAGCATCTGGATTCTGCCTAATAAAAAACATTTATTTTCATTGCAATGATGTATTTAA
ATTATTTCTGAATATTTTACTAAAAAGGGAATGTGGGAGGTCAGTGCATTTAAAACATAAAGAAATGAAGAGCTAGT
TCAAACCTTGGGAAAATACACTATATCTTAAACTCCATGAAAGAAGGTGAGGCTGCAAACAGCTAATGCACATTGGC
AACAGCCCTGATGCCTATGCCTTATTCATCCCTCAGAAAAGGATTCAAGTAGAGGCTTGATTTGGAGGTTAAAGTTT
TGCTATGCTGTATTTTACATTACTTATTGTTTTAGCTGTCCTCATGAATGTCTTTTCACTACCCATTTGCTTATCCT
GCATCTCTCAGCCTTGACTCCACTCAGTTCTCTTGCTTAGAGATACCACCTTTCCCCTGAAGTGTTCCTTCCATGTT
TTACGGCGAGATGGTTTCTCCTCGCCTGGCCACTCAGCCTTAGTTGTCTCTGTTGTCTTATAGAGGTCTACTTGAAG
AAGGAAAAACAGGGGGCATGGTTTGACTGTCCTGTGAGCCCTTCTTCCCTGCCTCCCCCACTCACAGTGACCCGGAA
TCTGCAGTGCTAGTCTCCCGGAACTATCACTCTTTCACAGTCTGCTTTGGAAGGACTGGGCTTAGTATGAAAAGTTA
GGACTGAGAAGAATTTGAAAGGGGGCTTTTTGTAGCTTGATATTCACTACTGTCTTATTACCCTATCATAGGCCCAC
CCCAAATGGAAGTCCCATTCTTCCTCAGGATGTTTAAGATTAGCATTCAGGAAGAGATCAGAGGTCTGCTGGCTCCC
TTATCATGTCCCTTATGGTGCTTCTGGCTCTGCAGTTATTAGCATAGTGTTACCATCAACCACCTTAACTTCATTTT
TCTTATTCAATACCTAGGTAGGTAGATGCTAGATTCTGGAAATAAAATATGAGTCTCAAGTGGTCCTTGTCCTCTCT
CCCAGTCAAATTCTGAATCTAGTTGGCAAGATTCTGAAATCAAGGCATATAATCAGTAATAAGTGATGATAGAAGGG
TATATAGAAGAATTTTATTATATGAGAGGGTGAAACCTAAAATGAAATGAAATCAGACCCTTGTCTTACACCATAAA
CAAAAATAAATTTGAATGGGTTAAAGAATTAAACTAAGACCTAAAACCATAAAAATTTTTAAAGAAATCAAAAGAAG
AAAATTCTAATATTCATGTTGCAGCCGTTTTTTGAATTTGATATGAGAAGCAAAGGCAACAAAAGGAAAAATAAAGA
AGTGAGGCTACATCAAACTAAAAAATTTCCACACAAAAAAGAAAACAATGAACAAATGAAAGGTGAACCATGAAATG
GCATATTTGCAAACCAAATATTTCTTAAATATTTTGGTTAATATCCAAAATATATAAGAAACACAGATGATTCAATA
ACAAACAAAAAATTAAAAATAGGAAAATAAAAAAATTAAAAAGAAGAAAATCCTGCCATTTATGCGAGAATTGATGA
ACCTGGAGGATGTAAAACTAAGAAAAATAAGCCTGACACAAAAAGACAAATACTACACAACCTTGCTCATATGTGAA
ACATAAAAAAGTCACTCTCATGGAAACAGACAGTAGAGGTATGGTTTCCAGGGGTTGGGGGTGGGAGAATCAGGAAA
CTATTACTCAAAGGGTATAAAATTTCAGTTATGTGGGATGAATAAATTCTAGATATCTAATGTACAGCATCGTGACT
GTAGTTAATTGTACTGTAAGTATATTTAAAATTTGCAAAGAGAGTAGATTTTTTTGTTTTTTTAGATGGAGTTTTGC
TCTTGTTGTCCAGGCTGGAGTGCAATGGCAAGATCTTGGCTCACTGCAACCTCCGCCTCCTGGGTTCAAGCAAATCT
CCTGCCTCAGCCTCCCGAGTAGCTGGGATTACAGGCATGCGACACCATGCCCAGCTAATTTTGTATTTTTAGTAGAG
ACGGGGTTTCTCCATGTTGGTCAGGCTGATCCGCCTCCTCGGCCACCAAAGGGCTGGGATTACAGGCGTGACCACCG
GGCCTGGCCGAGAGTAGATCTTAAAAGCATTTACCACAAGAAAAAGGTAACTATGTGAGATAATGGGTATGTTAATT
AGCTTGATTGTGGTAATCATTTCACAAGGTATACATATATTAAAACATCATGTTGTACACCTTAAATATATACAATT
TTTATTTGTGAATGATACCTCAATAAAGTTGAAGAATAATAAAAAAGAATAGACATCACATGAATTAAAAAACTAAA
AAATAAAAAAATGCATCTTGATGATTAGAATTGCATTCTTGATTTTTCAGATACAAATATCCATTTGACTGTTTACT
CTTTTCCAAAACAATACAATAAATTTTAGCACTTTATCTTCATTTTCCCCTTCCCAATCTATAATTTTATATATATA
TATTTTAGATATTTTGTATAGTTTTACTCCCTAGATTTTCTAGTGTTATTATTAAATAGTGAAGAAATGTTTACACT
TATGTACAAAATGTTTTGCATGCTTTTCTTCATTTCTAACATTCTCTCTAAGTTTATTCTATTTTTTCCTGATTATC
CTTAATATTATCTCTTTCTGCTGGAAATATATTGTTACTTTTGGTTTATCTAAAAATGGCTTCATTTTCTTCATTCT
AAAATCATGTTAAATTAATACCACTCATGTGTAAGTAAGATAGTGGAATAAATAGAAATCCAAAAACTAAATCTCAC
AAAATATAATAATGTGATATATAAAAATATAGCTTTTAAATTTAGCTTGGAAATAAAAAACAAACAGTAATTGAACA
ACTATACTTTTTGAAAAGAGTAAAGTGAAATGCTTAACTGCATATACCACAATCGATTACACAATTAGGTGTGAAGG
TAAAATTCAGTCACGAAAAAACTAGAATAAAAATATGGGAAGACATGTATATAATCTTAGAGATAACAGTGTTATTT
AATTATCAACCCAAAGTAGAAACTATCAAGGGAGAAATAAATTCAGTCAACAATAAAAGCATTTAAGAAGTTATTCT
AGGCTGGGAGCGGTGGCTCACACCTGCAATTGCAGCACTTTGGGAGGCCTAGACAGGCGGATCACGACGTCAGGAGT
TCAAGATCAGCCTGGCCAACATAGTGAAACCTCATCGCTACTAAAAATATAAAAACTTAGCCTGGCGTGGTGGCAGG
CATGTGTAATCCCAGCAATTTGGGAGGCTGAGGCAGGAGAATCGCTTGATCCTGGGAGGCAGAGGTTGCAGTGAGCC
AAGATTGTGCCACTGCATTCCAGCCCAGGTGACAGCATGAGACTCCGTCACAAAAAAAAAAGAAAAAAAAGGGGGGG
GGGGGCGGTGGAGCCAAGATGACCGAATAGGAACAGCTCCAGTCTATAGCTCCCATCGTGAGTGACGCAGAAGACGG
GTGATTTCTGCATTTCCAACTGAGGTACCAGGTTCATCTCACAGGGAAGTGCCAGGCAGTGGGTGCAGGACAGTAGT
GCAGTGCACTGTGCATGAGCCGAAGCAGGGCGAGGCATCACCTCACCCGGGAAGCACAAGGGGTCAGGGAATTCCCT
TTCCTAGTCAAAGAAAAGGGTGACAGATGGCACCTGGAAAATCGGGTCACTCCCGCCCTAATACTGCGCTCTTCCAA
CAAGCTTAACAAATGGCACACCAGGAGATTATATCCCATGCCTGGCTCAGAGGGTCCTACGCCCATGGAGCCTCGCT
CATTGCTAGCACAGCAGTCTGAGGTCAAACTGCAAGGTGGCAGTGAGGCTGGGGGAGGGGTGCCCACCATTGTCCAG
GCTTGAGCAGGTAAACAAAGCCGCCTGGAAGCTCGAACTGGGTGGAGCCCACCACAGCTCAAGGAGGCCTGCCTGCC
TCTGTAGGCTCCACCTCTAGGGGCAGGGCACAGACAAACAAAAGACAACAAGAACCTCTGCAGACTTAAATGTCCCT
GTCTGACAGCTTTGAAGAGAGTAGTGGTTCTCCCAGCACATAGCTTCAGATCTGAGAACAGGCAGACTGCCTCCTCA
AGTGGGTCCCTGACCCCCGAGTAGCCTAACTGGGAGGCATCCCCCAGTAGGGCGGACTGACACCTCACATGGCTGGT
ACTCCTCTAAGACAAAACTTCCAGAGGAATGATCAGGCAGCAGCATTTGCGGTTCACCAATATCCACTGTTCTGCAG
CCACCGCTGCTGATACCCAGGAAAACAGCATCTGGAGTGGACCTCCAGTAAACTCCAACAGACCTGCAGCTGAGGGT
CCTGACTGTTAGAAGGAAAACTAACAAACAGAAAGGACATCCACACCAAAAACCCATCTGTACATCACCATCATCAA
AGACCAAAGGTAGATAAAACCATAAAGATGGGGAAAAAGCAGAGCAGAAAAACTGGACACTCTAAAAATGAGAGTGC
CTCTCCTTCTCCAAAGTAACGCAGCTCCTCACCAGCAATGGAACAAAGCTGGGCAGAGAATGACTTTGACGAGTTGA
GAGAGGAAGGCTTCAGAAGATCAAACTACTCCAAGCTAAAGGAGGAAGTTCGAACAAACGGCAAAGAAGTAAAAAAC
TTTGAAAAAAAATTAGATGAATGGATAACTAGAATAACCAATGCACAGAAGTCCTTAAAGGACCTGATGGAGCTGAA
AACCAAGGCAGGAGAACTACGTGACAAATACACAAGCCTCAGTAACCGATGAGATCAACTGGAAGAAAGGGTATCAA
TGACGGAAGATGAAATGAATGAAATGAAGCATGAAGAGAAGTTTAGAGAAAAAAGAATAAAAAGAAACGAACAAAGC
CTCCAAGAAATATGGGACTATGTGAAAAGACCAAATCTACATCTAATTGGTGTAGCTGAAAGTGATGGGGAGAATGG
AACCAAGTTGGAAAACACTCTGCAGGATATTATCCAGGAGAACTTCCCCAATCTAGCAAGGCAGCCCAAATTCACAT
TCAGGAAATACAGAGAACGCCACAAAGATACTCCTAGAGAAAAGCAACTCCAAGACACATAACTGACAGATTCACCA
AAGTTGAAATGAAGGAAAAAATGTTAAGGGCAGCCAGAGAGAAAGGTCGGGTTACCCACAAAGGGAAGCCCATCAGA
CTAACAGCTGATCTATCGGCAGAAACTCTACAAGCCAGAAGAAAGTGGGGGCCAATATTCAACATTGTTAAAGAAAA
GAATTTTCGGCCCAGAATTTCATATCCAGCCAAACTAAGCTTCATAAGCATTGGAGAAATAAAATCCTTTACAGACA
AGCAAATGCTGAGAGATTTTGTCACCACCAGGCCTGCCCTACAAGAGCTCCTGAAGGAAGCACTAAACATGGAAAGG
AACAACTAGTATCAGCCACTGCAAAAACATGCCAAATTGTAAACGACCATCAAGGCTAGGAAGAAACTGCATCAAGG
AGCAAAATAACCAGCTAACATCATAATGACAGGATCAAATTCATACATAACAATACTCACCTTAAATGTAAATAGGC
TAAATGCTCCAATTAAAAGACACAGACTGGCAAATTGGATAAGGAGTCAAGACCCATCTGTCGTTATGTATTCAGGA
AACCCATCTCACGTGCAGAGACACACATAGGCTCGAAATAAAAGGATGGAGGAATATCTACCAAGCAAATGGAAAAC
AAAAAAAGGCAGGGGTTGCAATCCTAGTCTCTGATAAAACAGATTTTAAACCAACAAAGATCAAAAGAGACAAAGAA
GGCCATTACATAATGGCAAAGGGATCTATTCAAGAAGAAGAACTAACTATACTAAATATATATGCACCCAATACAGG
AGCACCCAGATTCATAAAACAAGTCCTGAGTGACCTACAAAGAGACTTAGATGCCCACACAATAATAATGGGAGACT
TTAACACCCCACTGTCAACATTAGACAGATCAACGAGACAGAAAGTTAACAAGGATATCCAGGAATTGGACTCAGCT
CTGCACCAAGCAGACCTAATAGACATCTACAGAACTCTCCACCCCAAATCAACAGAATATACATTCTTTTCAGCACC
ACACCACACCTATTCCAAAACTGACCACATAGTTGGAAGTAAAGCTCTCCTCAGCAAATGTAAAAGAACAGAAACTA
TAACAAACTGTCTCTCAGACCACAGTGCAATCAAACTAGAACTCAGGATTAAGAAACTCACTCAAAACCACTCAGCT
ACATGGAAACTGAACAGCCTGCTCCTGAATGACTACTGGGTACATAACAAAATGAAGGCAGAAATAAAGATGTTCTT
TGAAACAACGAGAACAAAGACACAACACACCAGAATCTCTGAGACACATTCAAAGCAGTGTGTAGAGGGAAATTTAT
AGCACTAAATGCCCACAAGGGAAAGCAGGAAAGATCTAAAATTGACACCCTAACATCACAATTAAAAAACTAGAGAA
GCAGGAGCAAACACATTCAAAAGCTAACAGAAGACAAGAAATAACTAAGATCAGAGCAGAAGTGAAGAAGATAGAGA
CACAAAAAACCCTTCAAAAAAATCAATGAATCCAGAAGCTGTTTTTTTGAAAAGATCAACAAAATTGATAGACTGCT
AGCAAGACTAATAAAGAAGAAAGGGGAGAAGAATCAAATAGACGCAATAAAAAATGACACGGGGTATCACCACTGAT
CCCACAGAAATACAAACTACCGTCAGAGAATACTATAAACACCTCTACGCAAATAAACTAGAAAATCTAGAAGAAAT
GGATAAATTCCTCGACACATACACTCTGCCAAGACTAAACCAGGAAGAAGTTGTATCTCTGAATAGACCAATAACAG
GCTCTGAAATTGAGGCAATAATTAATAGCTTATCAACCAAAAAAAGTCCGGGACCAGTAGGATTCATAGCCGAATTC
TACCAGAGGTACAAGGAGGAGCTGGTACCATTCCTTCTGAAACTATTCCAATCAATAGAAAAAGAGGGAATCCTCCC
TAACTCATTTTATGAGGCCAGCATCATCCTGATACCAAAGCCTGACAGAGACACAACAAAAAAAGAGAATGTTACAC
CAATATCCTTGATGAACATCGATGCAAAAATCCTCAATAAAATACTGGCAAACTGAATCCAGCAGCACATCAAAAAG
CTTATCCTCCATGATCAAGTGGGCTTCATCCCTGCCATGCAAGGCTGGTTCAACATACGAAATCAATAAACATAATC
CAGCATATAAACAGAACCAAAGACACAAACCATATGATTATCTCAATAGATGCAGAAAAGGCCTTTGACAAAATTCA
ACAATGCTTCATGCTAAAAACTCTCAATAAATTAGGTATTGATGGGACATATCTCAAAATAATAAGAGCTATCTATG
ACAAACCCACAGCCAATATCATACTGAGTGGACAAAAACTGGAAGCATTCCCTTTGAAAACTGGCACAAGGCAGGGA
TGCCCTCTCTCACCACTCCTATTCAACATAGTGTTGGAAGTTCTGGCCAGGGCAATCAGGCAGGAGAAGGAAATAAA
GGGCATTCAATTAGGAAAAGAGGAAGGTGAAATTGTCCCTGTTTGCAGATGACATGATTGTATATCTAGAAAACCCC
ATTGTCTCAGCCCAAAATCTCCTTAAGCTGATAAGCAACTTCAGCAAAGTCTCAGGATATAAAATCAGTGTGCAAAA
ATCACAAGTATTCCTATGCACCAATAACAGACAAACAGAGAGCCAAATCATGAGTGAACTCCCATTCACAATTGCTT
CAAAGAGAATAAAATACCTAGGAATCCAACTTACAAGGGATGTGAAGGACCTCTTCAAGGAGAACTACAAACCACTG
CTCAATGAAATAAAAGAGGATACAAACAAATGGAAGAACATTCCATGCTTATGGGTAGGAAGAATCATATCGTGAAA
ATGGTCATACTGCCCAAGGTAATTTATAGATTCAATGCCATCCCCATCAAGCTACCAATGACTTTCTTCACAGAACT
GGAAAAAACTACTTTAAAGTTCATATGGAATCAAAAAAGAGCCCACATCACCAAGGCAATCCTAAGCCAAAAGAACA
AAGCTGGAGGCATCACGCTACCTGACTTCAAACTATACTACAATGCTACGGTAACCAAAACAGCATGGTACTGGTAC
CAAAACAGAGATCTAGACCAATGGAACAGAACAGAGCCCTCAGAAATAATGCCGCATATCTACAACTATCCGATCTT
TGACAAACCTGAGAGAAACAAGCAATGGGGAAAGGATTCCCTATTTAATAAATGGTGCTGGGAAAACTGGCTAGCCA
TATGTAGAAAGCTGAAACTGGATCCTTCCTTACACCTTATACAAAAATTAATTCAAGATGGATTAAAGACTTAAACA
TTAGACCTAAAACCATAAAAACCCTAGAAAAAAACCTAGGCAATACCATTCAGGACATAGGCATGGGCAAGGACTTC
ATGTCTAAAACACCAAAACGAATGGCAACAAAAGACAAAATGGACAAACGGGATCTAATTAAACTAAAGAGCTTCTG
CACAGCTAAAGAAACTACCATCAGAGTGAACAGGCAACCTACAAAATGGGAGAAAATTTTTGCAATCTACTCATCTG
ACAAAGGGCTAATATCCAGAATCTACAATGAACTCAAACAAATTTACAAGAAAAAACAAACAACCCCATCAAAAAGT
GGGCAAAGGATATGAACAGACACTTCTCAAAAGAAGACATTTATGTAATCAAAAAACACATGAAAAAATGCTCATCA
TCACTAGCCATCAGAGAAATGCAAATCAAAACCACAATGAGATACCATCTCACACCAGTTAGAATGGCGATCATTAA
AAAGTCAGGAAACAACAGGTGCTGGAGAGGATGTGGAGAAACAGGAACAACTTTTACACTGTTGGTGGGACTGTAAA
CTAGTTCAACCATTGCGGAAGTCAGTGTGGCAATTCCTCAGGAATCTAGAACTAGAAATACCATTTGACCCAGCCAT
CCCATTACTGGGTAGATACCCAAAGGATTATAAATCATGCTGCTATAAAGACACATGCACACGTATGTTTATTGCAG
CACTATTCACAATAGCAAAGACTTGGAACCAACCCAAATGTCCAACAACGATAGATTGGATTAAGAAAATGTGGCAC
ATATACACCATGGAATACTATGCAGCCATAAAAAATGATGAGTTCATGTCCTTTGTAGGGACATGGATGAAGCTGGA
AACTATCATTCTCAGCAAACTATCACAAGGACAATAAACCAAACACCGCATGTTCTCACTCATAGGTGGGAATTGAA
CAATGAGAACACATGGACACATGAAGAGGAACATCACACTCTGGGGACTGTTATGGGGTGGGGGGCAGGGGCAGGGA
TAGCACTAGGAGATATACCTAATGCTAAATGACGAGTTAATGGGTGCAGCACACCAACATGGCACATGTATACATAT
ATAACAAACCTGCCGTTGTGCACATGTACCCTAAAACTTGAAGTATAATAATAAAAAAAAGTTATCCTATTAAAACT
GATCTCACACATCCGTAGAGCCATTATCAAGTCTTTCTCTTTGAAACAGACAGAAATTTAGTGTTTTCTCAGTCAGT
TAAC

NCBI Accession No. P68871 (SEQ ID NO: 250)

MVHLTPEEKSAVTALWGKVNVDEVGGEALGRLLVVYPWTQRFFESFGDLS
TPDAVMGNPKVKAHGKKVLGAFSDGLAHLDNLKGTFATLSELHCDKLHVD
PENFRLLGNVLVCVLAHHFGKEFTPPVQAAYQKVVAGVANALAHKYH

NCBI Accession No. NP_000509 (SEQ ID NO: 251)

MVHLTPEEKSAVTALWGKVNVDEVGGEALGRLLVVYPWTQRFFESFGDLS
TPDAVMGNPKVKAHGKKVLGAFSDGLAHLDNLKGTFATLSELHCDKLHVD
PENFRLLGNVLVCVLAHHFGKEFTPPVQAAYQKVVAGVANALAHKYH

NCBI Accession No. NP_269215 (SEQ ID NO: 252)

MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDELDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

NCBI Accession No. WP_011681470 (SEQ ID NO: 253)

MTKPYSIGLDIGTNSVGWAVTTDNYKVPSKKMKVLGNTSKKYIKKNLLGV
LLFDSGITAEGRRLKRTARRRYTRRRNRILYLQEIFSTEMATLDDAFFQR
LDDSFLVPDDKRDSKYPIFGNLVEEKAYHDEFPTIYHLRKYLADSTKKAD
LRLVYLALAHMIKYRGHFLIEGEFNSKNNDIQKNFQDFLDTYNAIFESDL
SLENSKQLEEIVKDKISKLEKKDRILKLFPGEKNSGIFSEFLKLIVGNQA
DFRKCFNLDEKASLHESKESYDEDLETLLGYIGDDYSDVFLKAKKLYDAI
LLSGELTVTDNETEAPLSSAMIKRYNEHKEDLALLKEYIRNISLKTYNEV
EKDDTKNGYAGYIDGKTNQEDFYVYLKKLLAEFEGADYFLEKIDREDFLR
KQRTFDNGSIPYQIHLQEMRAILDKQAKFYPFLAKNKERIEKILTFRIPY
YVGPLARGNSDFAWSIRKRNEKITPWNFEDVIDKESSAEAFINRMTSFDL
YLPEEKVLPKHSLLYETFNVYNELTKVRFIAESMRDYQFLDSKQKKDIVR
LYFKDKRKVTDKDIIEYLHAIYGYDGIELKGIEKQFNSSLSTYHDLLNII
NDKEFLDDSSNEAIIEEIIHTLTIFEDREMIKQRLSKFENIFDKSVLKKL
SRRHYTGWGKLSAKLINGIRDEKSGNTILDYLIDDGISNRNEMQLIHDDA
LSFKKKIQKAQIIGDEDKGNIKEVVKSLPGSPAIKKGILQSIKIVDELVK
VMGGRKPESIVVEMARENQYTNQGKSNSQQRLKRLEKSLKELGSKILKEN
IPAKLSKIDNNALQNDRLYLYYLQNGKDMYTGDDLDIDRLSNYDIDHIIP
QAFLKDNSIDNKVLVSSASNRGKSDDVPSLEVVKKRKTFWYQLLKSKLIS
QRKFDNLTKAERGGLSPEDKAGFIQRQLVETRQITKHVARLLDEKFNNKK
DENNRAVRTVKIITLKSTLVSQFRKDFELYKVREINDFHHAHDAYLNAVV
ASALLKKYPKLEPEFVYGDYPKYNSFRERKSATEKVYFYSNIMNIFKKSI
SLADGRVIERPLIEVNEETGESVWNKESDLATVRRVLSYPQVNVVKKVEE
QNHGLDRGKPKGLFNANLSSKPKPNSNENLVGAKEYLDPKKYGGYAGISN
SFTVLVKGTIEKGAKKKITNVLEFQGISILDRINYRKDKLNELLEKGYKD
IELIIELPKYSLFELSDGSRRMLASILSINNKRGEIHKGNQIFLSQKFVK
LLYHAKRISNTINENHRKYVENHKKEFEELFYYILEFNENYVGAKKNGKL
LNSAFQSWQNHSIDELCSSFIGPTGSERKGLFELTSRGSAADFEFLGVKI
PRYRDYTPSSLLKDATLIHQSVTGLYETRIDLAKLGEG

GenBank Accession No. ABD57356.1 (SEQ ID NO: 254)

MNEQAKQLVAVTRQPALNAGVGLVLAQLAEVEARQIPGSLAEARAHCLAQ
GAPDILLVEVENPQTLAADLAALAECCPPQMRLVLLGERGDVTLFRWLIS
VGVDDYYPAPLDPDALRTGLLRLLGVPLVTSLRKGRVICVVGAAGGVGTS
TVAANLAMALADQHHRQVALLDLNLHHSRHPILLGSDYAPPGEQWWQATD
RLDGTLLAHTAHQLGPRLFLFYDEGQELVLGAEQLVAAVNVMAEHYSTLI
IDVPDLRTHGLRALLQEADVVLWLHDFSLGALRLLGQCPQGGQAQRRLLV
GNHCRGKEGRVPAQELERVCGQPHAAVLPYDHGVFVRAERAGQPLIQQKS
KLARALTLLAGELVGAQVTGRGRR

GenBank Accession No. ANW61888.1 (SEQ ID NO: 255)

MAPKKKRKVMSQFDILCKTPPKVLVRQFVERFERPSGEKIASCAAELTYL
CWMITHNGTAIKRATFMSYNTIISNSLSFDIVNKSLQFKYKTQKATILEA
SLKKLIPAWEFTIIPYNGQKHQSDITDIVSSLQLQFESSEEADKGNSHSK
KMLKALLSEGESIWEITEKILNSFEYTSRFTKTKTLYQFLFLATFINCGR
FSDIKNVDPKSFKLVQNKYLGVIIQCLVTETKTSVSRHIYFFSARGRIDP
LVYLDEFLRNSEPVLKRVNRTGNSSSNKQEYQLLKDNLVRSYNKALKKNA
PYPIFAIKNGPKSHIGRHLMTSFLSMKGLTELTNVVGNWSDKRASAVART
TYTHQITAIPDHYFALVSRYYAYDPISKEMIALKDETNPIEEWQHIEQLK
GSAEGSIRYPAWNGIISQEVLDYLSSYINRRI

### OTHER EMBODIMENTS

While we have described a number of embodiments, it is apparent that our disclosure and examples also provide other embodiments that utilize or are encompassed by the compositions and methods described herein. Therefore, it will be appreciated that the scope of disclosure is to be defined by that which may be understood from the disclosure rather than by the specific embodiments that have been represented by way of example. Limitations described with respect to one aspect of the disclosure, in certain embodiments, be practiced with respect to other aspects of the disclosure. For example, limitations of claims that depend directly or indirectly from a certain independent claim presented herein serve as support for those limitations being presented in additional dependent claims of one or more other independent claims.

1. A method of selectively targeting a hematopoietic cell type, the method comprising administering to a subject or system an adenoviral vector, wherein the adenoviral vector comprises:
(a) a capsid comprising one or more viral polypeptides of an Ad3, Ad7, Ad11, Ad14, Ad16, Ad21, Ad34, Ad37, or Ad50 serotype, wherein the one or more viral polypeptides comprise one or more of a:
(i) fiber knob;
(ii) fiber shaft;
(iii) fiber tail;
(iv) penton; and
(v) hexon; and
(b) a double-stranded DNA genome comprising a heterologous nucleic acid payload.

2. The method of claim 1, wherein the genome further comprises:
(a) a 3′ ITR and a 5′ ITR, wherein each of the 3′ ITR and the 5′ ITR are of the viral polypeptide serotype; and
(b) a packaging sequence, wherein the packing sequence is of the viral polypeptide serotype.

3. The method of claim 1, wherein the hematopoietic cell type is or comprises:
(i) a terminally differentiated cell type;
(ii) a progenitor cell type;
(iii) common lymphoid progenitors (CLPs), T cells, NK cells, colony forming unit (CFU)-pre B cells, B cells, common myeloid progenitors (CMPs), granulocyte-macrophage progenitors (GMPs), CFU-M cells, monoblasts, monocytes, macrophages, CFU-G cells, myeloblasts, granulocytes, neutrophils, eosinophils, basophils, megakaryocyte-erythrocyte progenitors (MEPs), BFU-E cells, CFU-E cells, erythroblasts, erythrocytes, CFU-Mk cells, megakaryocytes, and/or platelets; or
(iv) a mammalian hematopoietic cell type, optionally wherein the mammalian hematopoietic cell type is a human hematopoietic cell type.

4.-5. (canceled)

6. The method of claim 1, wherein the method is a method of in vivo gene therapy, optionally wherein the subject is a mammalian subject, optionally wherein the mammalian subject is a human subject.

7.-8. (canceled)

9. The method of claim 6, wherein the method comprises:
(i) mobilization of hematopoietic cells of the subject prior to administration of the adenoviral vector;
(ii) administering one or more immunosuppression agents to the subject, optionally wherein the administration of the one or more immunosuppression agents is prior to the administration of the adenoviral vector; or
(iii) a combination thereof.

10. (canceled)

11. The method of claim 1, wherein the method is a method of ex vivo gene therapy, optionally wherein the system is or comprises a biological sample derived from a mammalian donor, optionally wherein the mammalian donor is a human donor.

12.-13. (canceled)

14. The method of claim 1, wherein the heterologous nucleic acid payload comprises a selectable marker, optionally wherein the selectable marker is MGMT^{P140K}.

15. The method of claim 14, wherein the method comprises administering a selecting agent to the subject, optionally wherein the selecting agent comprises O⁶BG and/or BCNU.

16. The method of claim 1, wherein the one or more viral polypeptides comprise the:
(a) fiber knob and fiber shaft;
(b) fiber knob and fiber tail;
(c) fiber knob and penton;
(d) fiber knob and hexon;
(e) fiber knob, hexon, and penton;
(f) fiber shaft and fiber tail;
(g) fiber shaft and penton;
(h) fiber shaft and hexon;
(i) fiber shaft, hexon, and penton;
(j) fiber tail and penton;
(k) fiber tail and hexon;
(l) fiber tail, hexon, and penton;
(m) fiber knob, fiber shaft, and fiber tail;
(n) fiber knob, fiber shaft, and penton;
(o) fiber knob, fiber shaft, and hexon;
(p) fiber knob, fiber shaft, hexon, and penton;
(q) fiber knob, fiber shaft, fiber tail, and penton;
(r) fiber knob, fiber shaft, fiber tail, penton, and hexon; or
(s) penton and hexon.

17. The method of claim 1, wherein:
(i) the fiber knob has a sequence that has at least 80% identity to a sequence selected from SEQ ID NOs: 15, 33, 51, 69, 87, 105, 123, 141, 159, 177, and 195;
(ii) the fiber shaft has a sequence that has at least 80% identity to a sequence selected from SEQ ID NOs: 14, 32, 50, 68, 86, 104, 122, 140, 158, 176, and 194;
(iii) the fiber tail has a sequence that has at least 80% identity to a sequence selected from SEQ ID NOs: 18, 36, 54, 72, 90, 108, 126, 144, 162, 180, and 198;
(iv) the penton has a sequence that has at least 80% identity to a sequence selected from SEQ ID NOs: 16, 34, 52, 70, 88, 106, 124, 142, 160, 178, and 196;
(v) the hexon has a sequence that has at least 80% identity to a sequence selected from SEQ ID NOs: 17, 35, 53, 71, 89, 107, 125, 143, 161, 179, and 197; or
(vi) a combination thereof.

18.-21. (canceled)

22. The method of claim 1, wherein the adenoviral vector comprises a fiber of the serotype of the viral polypeptides.

23. The method of claim 1, wherein the fiber has a sequence that has at least 80% identity to a sequence selected from SEQ ID NOs: 13, 31, 49, 67, 85, 103, 121, 139, and 157.

24. The method of claim 1, wherein the adenoviral vector is a chimeric vector characterized in that the capsid comprises at least one of a fiber knob, fiber shaft, fiber tail, hexon, or penton that is not of the serotype of the viral polypeptides.

25. The method of claim 1, wherein the adenoviral vector is a helper dependent vector.

26. The method of claim 1, wherein the heterologous nucleic acid payload encodes:
(i) a protein;
(ii) a chimeric antigen receptor (CAR), T cell receptor (TCR), antibody, or small RNA, optionally wherein the small RNA is an shRNA;
(iii) a gene editing enzyme or system, wherein the gene editing is selected from CRISPR editing, base editing, prime editing, and zinc finger nuclease editing;
(iv) an agent for treatment of a condition selected from adenosine deaminase deficiency (ADA), adrenoleukodystrophy (ALD), agammaglobulinemia, alpha-1 antitrypsin deficiency, congenital amegakaryocytic thrombocytopenia, amyotrophic lateral sclerosis (Lou Gehrig's disease), ataxia telangiectasia, Batten disease, Bernard-Soulier Syndrome, CD40/CD40L deficiency, chronic granulomatous disease, common variable immune deficiency (CVID), congenital thrombotic thrombocytopenic purpura (cTTP), cystic fibrosis, Diamond Blackfan anemia (DBA), DOCK 8 deficiency, dyskeratosis congenital, Fabry disease, Factor V Deficiency, Factor VII Deficiency, Factor X Deficiency, Factor XI Deficiency, Factor XII Deficiency, Factor XIII Deficiency, familial apolipoprotein E deficiency and atherosclerosis (ApoE), familial erythrophagocytic lymphohistiocytosis, Fanconi anemia (FA), Friedreich ataxia, Gaucher disease, Glanzmann thrombasthenia, glucosemia, glycogen storage disease, glycogen storage disease type I (GSDI), Gray Platelet Syndrome, hemophilia, hemophilia A, hemophilia B, hereditary hemochromatosis, Hurler's syndrome, hyper IgM, Hypogammaglobulinemia, Krabbe disease, major histocompatibility complex class II deficiency (MHC-II), maple syrup urine disease, metachromatic leukodystrophy (MLD), mucopolysaccharidoses, mucopolysaccharidosis type I (MPS I), MPS II (Hunter Syndrome), MPS III (Sanfilippo syndrome), MPS IV (Morquio syndrome), MPS V, MPS VI (Maroteaux-Lamy syndrome), MPS VII (sly syndrome), muscular dystrophy, Niemann-Pick disease, Parkinson's disease, paroxysmal nocturnal hemoglobinuria (PNH), pernicious anemia, phenylketonuria (PKU), Pompe disease, pulmonary alveolar proteinosis (PAP), pure red cell aplasia (PRCA), pyruvate kinase deficiency, refractory anemia, Shwachman-Diamond syndrome, selective IgA deficiency, severe aplastic anemia, severe combined immunodeficiency disease (SCID), Severe combined immunodeficiency due to adenosine deaminase deficiency (ADA-SCID), sickle cell anemia, sickle cell disease, sickle cell trait, Tay Sachs, thalassemia, thalassemia intermedia, von Gierke disease, von Willebrand Disease, Wiskott-Aldrich syndrome (WAS), X-linked agammaglobulinemia (XLA), X-linked severe combined immunodeficiency (SCID-X1), Zellweger syndrome, α-mannosidosis, β-mannosidosis, β-thalassemia, and/or β-thalassemia major; or
(v) a combination thereof.

27.-31. (canceled)

32. A hematopoietic cell comprising an adenoviral vector and an adenoviral vector genome,
wherein the adenoviral vector comprises a capsid comprises one or more viral polypeptides of an Ad3, Ad7, Ad11, Ad14, Ad16, Ad21, Ad34, Ad37, or Ad50 serotype, the one or more viral polypeptides comprising one or more of a:
(i) fiber knob;
(ii) fiber shaft;
(iii) fiber tail;
(iv) penton; and
(v) hexon,
wherein the adenoviral vector genome comprises a double-stranded DNA genome comprising a heterologous nucleic acid payload, and
wherein the hematopoietic cell is a common lymphoid progenitor (CLP), T cell, NK cell, colony forming unit (CFU)-pre B cell, B cell, common myeloid progenitor (CMP) cell, granulocyte-macrophage progenitor (GMP) cell, CFU-M cell, monoblasts, monocyte, macrophage, CFU-G cell, myeloblast, granulocyte, neutrophil, eosinophil, basophil, megakaryocyte-erythrocyte progenitor (MEP) cell, BFU-E cell, CFU-E cell, erythroblast, erythrocyte, CFU-Mk cell, megakaryocyte, and/or platelet.

33. A hematopoietic cell comprising an adenoviral vector genome,
wherein the adenoviral vector genome comprises:
(a) a 3′ ITR and a 5′ ITR, wherein the 3′ ITR and the 5′ ITR are each of the same serotype selected from Ad3, Ad7, Ad11, Ad14, Ad16, Ad21, Ad34, Ad37, and Ad50;
(b) a packaging sequence, wherein the packing sequence is of the same serotype as the 3′ ITR and a 5′ ITR; and
(c) a heterologous nucleic acid payload, and
wherein the hematopoietic cell is a common lymphoid progenitor (CLP), T cell, NK cell, colony forming unit (CFU)-pre B cell, B cell, common myeloid progenitor (CMP) cell, granulocyte-macrophage progenitor (GMP) cell, CFU-M cell, monoblasts, monocyte, macrophage, CFU-G cell, myeloblast, granulocyte, neutrophil, eosinophil, basophil, megakaryocyte-erythrocyte progenitor (MEP) cell, BFU-E cell, CFU-E cell, erythroblast, erythrocyte, CFU-Mk cell, megakaryocyte, and/or platelet.

34. (canceled)

35. A method of in vivo gene therapy in a mammalian subject, the method comprising administering to the subject an adenoviral vector, wherein the adenoviral vector comprises:
(a) a capsid comprising one or more viral polypeptides of an Ad3, Ad7, Ad11, Ad14, Ad16, Ad21, Ad34, Ad37, or Ad50 serotype, wherein the one or more viral polypeptides comprise one or more of a:
(i) fiber knob;
(ii) fiber shaft;
(iii) fiber tail;
(iv) penton; and
(v) hexon; and
(b) a double-stranded DNA genome comprising a heterologous nucleic acid payload.

36.-40. (canceled)

41. An adenoviral donor vector comprising:
(a) a capsid comprising one or more viral polypeptides of an Ad3, Ad7, Ad11, Ad14, Ad16, Ad21, Ad34, Ad37, or Ad50 serotype, wherein the one or more viral polypeptides comprise one or more of a:
(i) fiber knob;
(ii) fiber shaft;
(iii) fiber tail;
(iv) penton; and
(v) hexon; and
(b) a double-stranded DNA genome comprising a heterologous nucleic acid payload.

42.-53. (canceled)

54. An adenoviral donor vector genome comprising:
(a) a 3′ ITR and a 5′ ITR, wherein the 3′ ITR and the 5′ ITR are each of the same serotype selected from Ad3, Ad7, Ad11, Ad14, Ad16, Ad21, Ad34, Ad37, and Ad50;
(b) a packaging sequence, wherein the packing sequence is of the ITR serotype; and
(c) a heterologous nucleic acid payload.

55.-60. (canceled)

61. A pharmaceutical composition comprising an adenoviral vector of claim 41, wherein the pharmaceutical composition is formulated for injection to a subject in need thereof.

62. The method of claim 1, wherein:
(i) the capsid comprises one or more viral polypeptides of an Ad7, Ad11, Ad16, or Ad34 serotype, and wherein the hematopoietic cell type is or comprises monocytes, optionally wherein the monocytes are CD11+/CD14+ monocytes;
(ii) the capsid comprises one or more viral polypeptides of an Ad7, Ad11, Ad16, or Ad34 serotype, and wherein the hematopoietic cell type is or comprises T cells, optionally wherein the T cells are CD3+ T cells;
(iii) the capsid comprises one or more viral polypeptides of an Ad7, Ad11, Ad16, or Ad34 serotype, and wherein the hematopoietic cell type is or comprises NK cells, optionally wherein the NK cells are CD3−/CD56+ NK cells: or
(iv) the capsid comprises one or more viral polypeptides of an Ad7, Ad11, Ad16, or Ad34 serotype, and wherein the hematopoietic cell type is or comprises B cells, optionally wherein the B cells are CD20+ B cells.

63.-76. (canceled)
